(19) United States
(12) Patent Application Publication
(10) Pub. No.: US 2010/0233146 A1
(43) Pub. Date: Sep. 16, 2010

(54) Coatings and Surface Treatments Having Active Enzymes and Peptides

(75) Inventor: C. Steven McDaniel, Austin, TX (US)

Correspondence Address: C. Steven McDaniel;c/o Daffer McDaniel, LLP, P.O. Box 684908, Austin, TX, 78768-4908, US

(73) Assignee: REACTIVE SURFACES, LTD., Austin, TX (US)

(21) Appl. No.: 12/474,921

(22) Filed: May 29, 2009

Related U.S. Application Data
(63) Continuation-in-part of application No. 10/884,355, filed on Jul. 2, 2004.
Application No. 10/884,355 is a continuation-in-part of application No. 12/243,755, filed on Oct. 1, 2008.
Application No. 12/243,755 is a continuation-in-part of application No. 10/655,345, filed on Sep. 4, 2003.
(60) Provisional application No. 61/057,705, filed on May 30, 2008.
(60) Provisional application No. 61/058,025, filed on Jun. 2, 2008.
(60) Provisional application No. 60/485,234, filed on Jul. 3, 2003.
(60) Provisional application No. 60/976,676, filed on Oct. 1, 2007.
(60) Provisional application No. 60/409,102, filed on Sep. 9, 2002.

(51) Int. Cl. A01N 63/00; C12N 9/16; C12N 9/00; C12N 9/78; C12N 9/48; C12N 9/76; C07K 7/06; C07K 7/08; C07K 5/10; C07K 5/08; C07K 14/00; A01P 1/00; C12N 11/16; B05D 3/00

(52) U.S. Cl. 424/94.2; 435/196; 435/183; 435/227; 435/212; 435/213; 530/329; 530/330; 530/331; 530/324; 530/327; 530/326; 435/174; 427/331; 427/352

(57) ABSTRACT

Disclosed herein are a materials such as a coating, an elastomer, an adhesive, a sealant, a textile finish, a wax, and a filler for such a material, wherein the material includes an enzyme such as an esterase (e.g., a lipolytic enzyme, a sulfuric ester hydrolase, an organophosphorus compound degradation enzyme), an enzyme that degrades a cell wall and/or a cell membrane component (e.g., a lysozyme, a lytic transgrycosylase, a peptidase), and/or a biocidal or biostatic peptide. Also disclosed herein are methods of decontaminating a surface comprising such a material from a chemical substrate of an enzyme such as a lipid or an organophosphorus compound, as well as reducing the growth of a microorganism on or within such a material.

### PRIORITY CLAIM

The present application claims priority to U.S. Provisional Application No. 61/057,705, filed May 30, 2008 and U.S. Provisional Application No. 61/058,025, filed Jun. 2, 2008. The present application is further a Continuation-in-Part of U.S. patent application Ser. No. 10/884,355 filed Jul. 2, 2004 which claims priority to U.S. Provisional Patent Application No. 60/485,234 filed Jul. 3, 2003. The present application is further a Continuation-in-Part of U.S. patent application Ser. No. 12/243,755 filed Oct. 1, 2008 which claims priority to U.S. Provisional Patent Application No. 60/976,676 filed Oct. 1, 2007. The present application is further a Continuation-in-Part of U.S. patent application Ser. No. 10/655,345 filed Sep. 4, 2003 which claims priority to U.S. Provisional Application No. 60/409,102 filed Sep. 9, 2002.

### BACKGROUND OF THE INVENTION

A. Field of the Invention

The invention relates generally to an active enzyme such as an esterase (e.g., a lipolytic enzyme, a sulfuric ester hydrolase, an organophosphorus compound degradation enzyme); an antifungal or antimicrobial peptide; an enzyme (e.g., a lysozyme, a lytic transglycosylase), that may degrade a cell wall, a viral proteinaceous molecule, and/or a biologial membrane (e.g., a cell membrane, a virus envelope); and/or a peptidase, in a composition and methods for using the same. The composition may comprise a surface treatment such as a coating, an elastomer, an adhesive, a sealant, a textile finish or a wax; or a filler typically used in such a surface treatment.

B. Description of the Related Art

The surface of a material may be subject to addition of a surface treatment such as a coating, an adhesive, a sealant, a textile finish, and/or a wax, with a surface treatment typically used, for example, to protect, decorate, attach, and/or seal a surface and/or the underlying material. A filler typically comprises a particulate material that may be used as a component of a surface treatment. An example of use of such items includes a coating such as paint comprising a filler forming a solid protective, decorative, or functional adherent film on a surface.

A biomolecule comprises a molecule often produced and isolated from an organism, such as an enzyme which catalyzes a chemical reaction. An example of an enzyme comprises a lipolytic enzyme (e.g., a lipase) that catalyzes a reaction on a lipid substrate, such as a vegetable oil, a phospholipid, a sterol, and other hydrophobic molecule. Often a lipolytic enzyme catalyzed reaction may be used for an industrial or a commercial purpose, such as an alcohol or an acid esterification, an interesterification, a transesterification, an acidolysis, an alcoholysis, and/or resolution of a racemic alcohol and an organic acid mixture.

Examples of an enzyme that detoxifies an organophosphorus compound (“organophosphate compound,” “OP compound”) include an organophosphorus hydrolase (“OPH”), an organophosphorus acid anhydrolase (“OPAA”), and a DFPase. Organophosphorus compounds and organosulfur (“OS”) compounds are used extensively as insecticides and are toxic to many organisms, including humans. OP compounds function as nerve agents. OP compounds have been used both as pesticides and chemical warfare agents.

Alexander Fleming discovered lysozyme during a search for antibiotics when adding a drop of mucus to a growing bacterial culture and discovered it killed the bacteria. Lysozymes have widespread distribution in animals and plants. A lysozyme serves as a “natural antibiotic” protecting fluids and tissues that are rich in potential food for bacterial growth, such as an egg white. As a part of the innate defense mechanism, lysozyme may be found in many mammalian secretions and tissues, saliva, tears, milk, cervical mucus, leucocytes, kidneys, etc. Other enzymes possess antibiotic activity.

A sulfuric ester hydrolase catalyzes a reaction at a sulfuric ester bond. A peptidase catalyzes a reaction at a peptide bond, such as a bond found in a peptide, a polypeptide or a protein, and may function as a digestive enzyme. Other enzymes catalyze various reactions.

### SUMMARY OF THE INVENTION

In general, the invention features a composition, comprising an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; wherein the composition comprises an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof. In some embodiments, the active enzyme comprises a plurality of active enzymes.

In certain embodiments, the enzyme comprises an esterase, a ceramidase, or a combination thereof, and wherein the esterase comprises a lipolytic enzyme, a phosphoric triester hydrolase, a sulfuric ester hydrolase, or a combination thereof. In some aspects, the lipolytic enzyme, the ceramidase, or a combination thereof, comprises a carboxylesterase, a lipase, a lipoprotein lipase, an acylglycerol lipase, a hormone-sensitive lipase, a phospholipase A₁, a phospholipases A₂, a phosphatidylinositol deacylase, a phospholipase C, a phospholipase D, a phosphoinositide phospholipase C, a phosphatidate phosphatase, a lysophospholipase, a sterol esterase, a galactolipase, a sphingomyelin phosphodiesterase, a sphingomyelin phosphodiesterases D, a ceramidase, a wax-ester hydrolase, a fatty-acyl-ethyl-ester synthase, a retinyl-palmitate esterase, a 11-cis-retinyl-palmitate hydrolase, an all-trans-retinyl-palmitate hydrolase, a cutinase, an acyloxyacyl hydrolase, or a combination thereof. In some facets, the lipolytic enzyme, the ceramidase, or a combination thereof comprises: a carboxylesterase derived from Actinidia deliciosa, Aedes aegypti, Aeropyrum pernix, Alicyclobacillus acidocaldarius, Aphis gossypii, Arabidopsis thaliana, Archaeoglobus fulgidus, Aspergillus clavatus, Athalia rosae, Bacillus acidocaldarius, Bombyx mandarina, Bombyx mori, Bos taurus, Burkholderia gladioli, Caenorhabditis elegans, Canis familiaris, Cavia porcellus, Chloroflexus aurantiacus, Felis catus, Fervidobacterium nodosum, Helicoverpa armigera, Homo sapiens, Macaca fascicularis, Malus pumila, Mesocricetus auratus, Mus musculus, Musca domestica, Mycoplasma hyopneumoniae, Myxococcus xanthus, Neosartorya fischeri, Oryctolagus cuniculus, Paeonia suffruticosa, Pseudomonas aeruginosa, Rattus norvegicus, Rubrobacter xylanophilus, Spodoptera exigua, Spodoptera litura, Sulfolobus acidocaldarius, Sulfolobus shibatae, Sulfolobus solfataricus, Sus scrofa, Thermotoga maritime, Thermus thermophilus, Vaccinium corymbosum, Vibrio harveyi, Xenopsylla cheopis, Yarrowia lipolytica, or a combination thereof; a lipase derived from Acinetobacter, Aedes aegypti, Anguilla japonica, Antrodia cinnamomea, Arabidopsis rosette, Arabidopsis thaliana, Arxula adeninivorans, Aspergillus niger, Aspergillus oryzae, Aspergillus tamarii, Aureobasidium pullulans, Avena sativa, Bacillus lichenifonnis, Bacillus sphaericus, Bacillus stearothermophilus, Bacillus subtilis, Bacillus thermocatenulatus, Bacillus thermoleovorans, Bombyx mandarina, Bombyx mori, Bos Taurus, Brassica napus, Brassica rapa, Burkholderia cepacia, Caenorhabditis elegans, Candida albicans, Candida antarctica, Candida deformans, Candida parapsilosis, Candida rugosa, Candida thermophila, Canis domesticus, Chenopodium rubrum, Clostridium beijerinckii, Clostridium botulinum, Clostridium novyi, Danio rerio, Galactomyces geotrichum, Gallus gallus, Geobacillus, Gibberella zeae, Gossypium hirsutum, Homo sapiens, Kurtzmanomyces sp., Leishmania infantum, Lycopersicon esculentum L, Malassezia furfur, Methanosarcina acetivorans, Mus musculus, Mus spretus, Mycobacterium tuberculosis, Mycoplasma hyopneumoniae, Myxococcus xanthus, Neosartorya fischeri, Oryctolagus cuniculus, Oryza sativa, Penicillium cyclopium, Phlebotomus papatasi, Pseudomonas aeruginosa, Pseudomonas fluorescens, Pseudomonas fragi, Pseudomonas sp, Rattus norvegicus, Rhizomucor miehei, Rhizopus oryzae, Rhizopus stolonifer, Ricinus communis, Samia cynthia ricini, Schizosaccharomyces pombe, Serratia marcescens, Spermophilus tridecemlineatus, Staphylococcus simulans, Staphylococcus xylosus, Sulfolobus solfataricus, Sus scrofa, Thermomyces lanuginosus, Trichomonas vaginalis, Vibrio harveyi, Xenopus laevis, Yarrowia lipolytica, or a combination thereof; a lipoprotein lipase derived from Capra hircus, Danio rerio, Felis catus, Homo sapiens, Mesocricetus auratus, Mus musculus, Oncorhynchus mykiss, Pagrus major, Papio Anubis, Rattus norvegicus, Sparus aurata, Sus scrofa, Thunnus orientalis, or a combination thereof; an acylglycerol lipase derived from Bacillus sp., Danio rerio, Homo sapiens, Leishmania infantum, Mus musculus, Mycobacterium tuberculosis, Penicillium camembertii, Rattus norvegicus, Solanum tuberosum, or a combination thereof; a hormone sensitive lipase derived from Bos Taurus, Homo sapiens, Mus musculus, Rattus norvegicus, Spermophilus tridecemlineatus, Sus scrofa, Tetrahymena thermophila, or a combination thereof; a phospholipase A₁ derived from Arabidopsis, Aspergillus oryzae, Bos Taurus, Brassica rapa, Caenorhabditis elegans, Capsicum annuum, Danio rerio, Homo sapiens, Mus musculus, Nicotiana tabacum, Polistes annularis, Polybia paulista, Rattus norvegicus, Serratia sp., Vespula vulgaris, or a combination thereof; a phospholipase A₂ derived from Acanthaster planci, Adamsia carciniopado, Aedes aegypti, Aeropyrum pernix, Aipysurus eydouxii, Apis mellifera, Arabidopsis thaliana, Aspergillus nidulans, Austrelaps superbus, Bitis gabonica, Bos taurus, Bothriechis schlegelii, Bothrops jararacussu, Brachylanio rerio, Bungarus caeruleus, Bungarus fasciatus, Canis familiaris, Cavia sp., Cerrophidion godmani, Chlamydomonas reinhardtii, Chrysophrys major, Crotalus viridis viridis, Daboia russellii, Danio rerio, Drosophila melanogaster, Echis carinatus, Echis ocellatus, Echis pyramidum leakeyi, Emericella nidulans, Equus caballus, Gallus gallus, Homo sapiens, Lapemis hardwickii, Laticauda semifasciata, Micrurus corallines, Mus musculus, Mytilus edulis, Naja kaouthia, Naja naja, Naja naja sputatrix, Nicotiana tabacum, Ophiophagus hannah, Ornithodoros parkeri, Oryctolagus cuniculus, Pagrus major, Patiria pectinifera, Polyandrocarpa misakiensis, Protobothrops mucrosquamatus, Rattus norvegicus, Sistrurus catenatus tergeminus, Trimeresurus borneensis, Trimeresurus flavoviridis, Trimeresurus gracilis, Trimeresurus gramineus, Trimeresurus okinavensis, Trimeresurus puniceus, Trimeresurus stejnegeri, Tuber borchii, Urticina crassicornis, Vipera russelli siamensis, Xenopus laevis, Xenopus tropicalis, or a combination thereof; a phospholipase C derived from Aedes aegypti, Aplysia californica, Arabidopsis thaliana, Asterina miniata, Bacillus cereus, Bacillus thuringiensis, Bos taurus, Caenorhabditis elegans, Chaetopterus pergamentaceus, Chlamydomonas reinhardtii, Coturnix japonica, Danio rerio, Dictyostelium discoideum, Drosophila melanogaster, Gallus gallus, Homarus americanus, Homo sapiens, Loligo pealei, Lytechinus pictus, Meleagris gallopavo, Misgurnus mizolepis, Mus musculus, Nicotiana tabacum, Oryza sativa, Oryzias latipes, Petunia inflate, Pichia stipitis, Pisum sativum, Plasmodium falciparum, Rattus norvegicus, Strongylocentrotus purpuratus, Sus scrofa, Torenia fournieri, Toxoplasma Watasenia scintillans, Xenopus laevis, Zea mays, or a combination thereof; a phospholipase D derived from Aedes aegypti, Arabidopsis thaliana, Arachis hypogaea, Bos taurus, Brassica oleracea, Caenorhabditis elegans, Cricetulus griseus, Cucumis melo var. inodorus, Cucumis sativus, Dictyostelium discoideum, Drosophila melanogaster, Emericella nidulans, Fragaria ananassa, Gossypium hirsutum, Homo sapiens, Lolium temulentum, Lycopersicon esculentum, Mus musculus, Oryza sativa, Papaver somniferum, Paralichthys olivaceus, Pichia stipitis, Pimpinella brachycarpa, Rattus norvegicus, Ricinus communis, Streptoverticillium cinnamoneum, Vigna unguiculata, Vitis vinifera, Zea mays, or a combination thereof; a phosphoinositide phospholipase C derived from Arabidopsis thaliana, Aspergillus clavatus, Aspergillus fumigatus, Brassica napus, Homo sapiens, Leishmania infantum, Mus musculus, Neosartorya fischeri, Physcomitrella patens, Pichia stipitis, Rattus norvegicus, Toxoplasma gondii, Trypanosoma brucei, Vigna unguiculata, Xenopus tropicalis, Zea mays, or a combination thereof; a phosphatidate phosphatase derived from Saccharomyces cerevisiae, or a combination thereof; a lysophospholipase derived from Aedes aegypti, Argas monolakensis, Aspergillus clavatus, Aspergillus fumigatus, Bos Taurus, Cavia porcellus, Clonorchis sinensis, Danio rerio, Dictyostelium discoideum, Emericella nidulans, Giardia lamblia, Homo sapiens, Monodelphis domestica, Mus musculus, Neosartorya fischeri, Pichia jadinii, Pichia stipitis, Rattus norvegicus, Schistosoma japonicum, Schizosaccharomyces pombe, Sclerotinia sclerotiorum, Xenopus tropicalis, or a combination thereof; a sterol esterase derived from Candida rugosa, Homo sapiens, Melanocarpus albomyces, Rattus norvegicus, or a combination thereof; a galactolipase derived from Homo sapiens, Solanum tuberosum, Vigna unguiculata, or a combination thereof; a sphingomyelin phosphodiesterase derived from Bacillus cereus, Homo sapiens, Pseudomonas sp., or a combination thereof; a ceramidase derived from Homo sapiens, Pseudomonas, or a combination thereof; a cutinase derived from Fusarium solani pisi, Monilinia fructicola, Pseudomonas putida, or a combination thereof; a retinyl palmitate esterase derived from Bos Taurus; or a combination thereof.

In other facets, the lipolytic enzyme comprises: a thermophilic carboxylesterase derived from Aeropyrum pernix, Alicyclobacillus acidocaldarius, Archaeoglobus fulgidus, Bacillus acidocaldarius, Pseudomonas aeruginosa, Sulfolobus shibatae, Sulfolobus solfataricus, Thermotoga maritime, or a combination thereof; a thermophilic lipase derived from Acinetobacter calcoaceticus, Acinetobacter sp., Bacillus sphaericus, Bacillus stearothermophilus, Bacillus thermocatenulatus, Bacillus thermoleovorans, Candida rugosa, Candida thermophila, GeoBacillus thermoleovorans Toshki, Pseudomonas fragi, Staphylococcus xylosus, Sulfolobus solfataricus, or a combination thereof; a psychrophilic lipase derived from Pseudomonas fluorescens; or a combination thereof; a thermophilic phospholipase A₂ derived from Aeropyrum pernix; a thermophilic phospholipase C derived from Bacillus cereus; or a combination thereof.

In certain aspects, the phosphoric triester hydrolase comprises an aryldialkylphosphatase, a diisopropyl-fluorophosphatase, or a combination thereof. In other facets, the aryldialkylphosphatase comprises an organophosphorus hydrolase, a human paraoxonase, an animal carboxylase, or a combination thereof; wherein the diisopropyl-fluorophosphatase comprises an organophosphorus acid anhydrolase, a squid-type DFPase, a Mazur-type DFPase, or a combination thereof; or a combination thereof of the forgoing. In particular facets, the organophosphorus hydrolase comprises an Agrobacterium radiobacter P230 organophosphate hydrolase, a Flavobacterium balustinum parathion hydrolase, a Pseudomonas diminuta phosphotriesterase, a Flavobacterium sp opd gene product, a Flavobacterium sp. parathion hydrolase opd gene product, or a combination thereof; wherein the animal carboxylase comprises an insect carboxylase; or a combination thereof; wherein the organophosphorus acid anhydrolase comprises an Altermonas organophosphorus acid anhydrolase, a prolidase, or a combination thereof; wherein the squid-type DFPase comprises a Loligo vulgaris DFPase, a Loligo pealei DFPase, a Loligo opalescens DFPase, or a combination thereof; wherein the Mazur-type DFPase comprises a mouse liver DFPase, a hog kidney DFPase, a Bacillus stearothermophilus strain OT DFPase, an Escherichia coli DFPase, or a combination thereof; or a combination thereof the forgoing.

In additional facets, the insect carboxylase comprises a Plodia interpunctella carboxylase, Chrysomya putoria carboxylase, Lucilia cuprina carboxylase, Musca domestica carboxylase, or a combination thereof; wherein the Altermonas organophosphorus acid anhydrolase comprises an Alteromonas sp JD6.5 organophosphorus acid anhydrolase, an Alteromonas haloplanktis organophosphorus acid anhydrolase, an Altermonas undina organophosphorus acid anhydrolase, or a combination thereof; wherein the prolidase comprises a human prolidase, a Mus musculus prolidase, a Lactobacillus helveticus prolidase, an Escherichia coli prolidase, an Escherichia coli aminopeptidase P, or a combination thereof; wherein the phosphoric triester hydrolase comprises a Plesiomonas sp. strain M6 mpd gene product, a Xanthomonas sp. phosphoric triester hydrolase, a Tetrahymena phosphoric triester hydrolase, or a combination thereof; or a combination thereof the forgoing.

In certain embodiments, the sulfuric ester hydrolase comprises an arylsulfatase. In other embodiments, the peptidase comprises a trypsin, a chymotrypsin, or a combination thereof. In particular embodiments, the antibiological enzyme comprises a lysozyme, a lysostaphin, a libiase, a lysyl endopeptidase, a mutanolysin, a cellulase, a chitinase, an α-agarase, an β-agarase, a N-acetylmuramoyl-L-alanine amidase, a lytic transglycosylase, a glucan endo-1,3-β-D-glucosidase, an endo-1,3(4)-β-glucanase, a metalloendopeptidase, a 3-deoxy-2-octulosonidase, a peptide-N4-(N-acetyl-β-glucosaminyhasparagine amidase, a mannosyl-glycoprotein endo-β-N-acetylglucosaminidase, a l-carrageenase, a κ-carrageenase, a λ-carrageenase, an α-neoagaro-oligosaccharide hydrolase, an endolysin, an autolysin, a mannoprotein protease, a glucanase, a mannase, a zymolase, a lyticase, a lipolytic enzyme, a peroxidase, or a combination thereof. In other embodiments, the antibiological peptidic agent comprises SEQ ID No. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or a combination thereof. In some aspects, the antibiological peptidic agent comprises a plurality of antibiological peptidic agents.

In other embodiments, the active enzyme comprises a mesophilic enzyme, a psychrophilic enzyme, a thermophilic enzyme, a halophilic enzyme, or a combination thereof. In some aspects, the active enzyme, the antibiological peptidic agent, or a combination thereof, comprises an immobilization carrier. In other aspects, the active enzyme, the antibiological peptidic agent, or a combination thereof, comprises a purified active enzyme, a purified antibiological peptidic agent, or a combination thereof,

In some embodiments, the active enzyme, the antibiological peptidic agent, or a combination thereof, comprises a particulate material. In some aspects, the active enzyme, the antibiological peptidic agent, or a combination thereof, comprises a cell-based particulate material. In other aspects, the cell-based particulate material comprises a whole cell particulate material or a cell fragment particulate material. In some facets, the average wet molecular weight or dry molecular weight of a primary particle of the particulate material is about 50 kDa to about 1.5×10¹⁴ kDa. In other facets, an average active enzyme content, an average antibiological peptidic agent content, or a combination thereof, per primary particle of the particulate material is about 0.01% to about 100%.

In other embodiments, the active enzyme, the antibiological peptidic agent, or a combination thereof, is attenuated, sterilized, or a combination thereof. In certain aspects, the active enzyme, the antibiological peptidic agent, or a combination thereof, comprises about 0.01% to about 80% of the composition by weight or volume. In other facets, the active enzyme, the antibiological peptidic agent, or a combination thereof, is microencapsulated.

In certain embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, is about 5 um to about 5000 um thick upon a surface. In some aspects, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a paint. In other aspects, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a clear coating. In particular facets, the clear coating comprises a lacquer, a varnish, a shellac, a stain, a water repellent coating, or a combination thereof.

In other embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a multicoat system. In some aspects, the multicoat system comprises 2 to 10 layers. In other aspects, a plurality of layers of the multicoat system comprise the active enzyme. In additional aspects, the multicoat system comprises a sealer, a water repellent, a primer, an undercoat, a topcoat, or a combination thereof. In some facets, the topcoat comprises the active enzyme.

In certain embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a coating that is capable of film formation. In some aspects, film formation occurs between about −10° C. to about 40° C. In other aspects, film formation occurs at baking conditions. In certain facets, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a volatile component and a non-volatile component, and wherein film formation occurs by loss of part of the volatile component. In other facets, film formation occurs by cross-linking of a binder.

In certain embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, produces a self-cleaning film. In other embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, produces a temporary film. In additional embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a non-film forming coating. In particular aspects, the non-film forming coating comprises a non-film formation binder. In some facets, the non-film forming coating comprises a coating component in a concentration that is insufficient to produce a solid film.

In some embodiments, the architectural coating comprises an architectural wood coating, an architectural masonry coating, an architectural artist's coating, an architectural plastic coating, an architectural metal coating, or a combination thereof. In certain aspects, the architectural coating has a pot life of at least 12 months at about −10° C. to about 40° C.

In other embodiments, the composition comprises an automotive coating, a can coating, a sealant coating, or a combination thereof. In some embodiments, the composition comprises a chemical agent resistant coating, a camouflage coating, a pipeline coating, a traffic marker coating, an aircraft coating, a nuclear power plant coating, or a combination thereof.

In particular embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a coating for a plastic surface.

In other embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a water-borne coating. In some aspects, the water-borne coating comprises a latex coating. In additional embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a solvent-borne coating.

In certain embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, has a low-shear viscosity of about 100 P to about 3000 P, has a medium-shear viscosity of about 84 Ku and about 140 Ku, has a high-shear viscosity of about 0.5 P to about 2.5 P, or a combination thereof.

In some embodiments, the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a binder, a liquid component, a colorant, an additive, or a combination thereof. In many aspects, the binder comprises a thermoplastic binder, a thermosetting binder, or a combination thereof. In some facets, the binder comprises an oil-based binder, a polyester resin, a modified cellulose, a polyamide, an amino resin, a urethane binder, a phenolic resin, an epoxy resin, a polyhydroxyether binder, an acrylic resin, a polyvinyl binder, a rubber resin, a bituminous binder, a polysulfide binder, a silicone binder, an organic binder, or a combination thereof. In other facets, the oil-based binder comprises an oil, an alkyd, an oleoresinous binder, a fatty acid epoxide ester, or a combination thereof; wherein the polyester resin comprises a hydroxy-terminated polyester, a carboxylic acid-terminated polyester, or a combination thereof; wherein the modified cellulose comprises a cellulose ester, a nitrocellulose, or a combination thereof; wherein the epoxy resin comprises a cycloaliphatic epoxy binder; wherein the rubber resin comprises a chlorinated rubber resin, a synthetic rubber resin, or a combination thereof; or a combination thereof the forgoing.

In certain aspects, the liquid component comprises a solvent, a thinner, a diluent, a plasticizer, or a combination thereof. In other facets, the liquid component comprises a liquid organic compound, an inorganic compound, water, or a combination thereof. In some facets, the liquid organic compound comprises a hydrocarbon, an oxygenated compound, a chlorinated hydrocarbon, a nitrated hydrocarbon, a miscellaneous organic liquid, a plasticizer, or a combination thereof; wherein the inorganic compound comprises ammonia, hydrogen cyanide, hydrogen fluoride, hydrogen cyanide, sulfur dioxide, or a combination thereof; wherein the water comprises methanol, ethanol, propanol, isopropyl alcohol, tert-butanol, ethylene glycol, methyl glycol, ethyl glycol, propyl glycol, butyl glycol, ethyl diglycol, methoxypropanol, methyldipropylene glycol, dioxane, tetrahydrorfuran, acetone, diacetone alcohol, dimethylformamide, dimethyl sulfoxide, ethylbenzene, tetrachloroethylene, p-xylene, toluene, diisobutyl ketone, tricholorethylene, trimethylcyclohexanol, cyclohexyl acetate, dibutyl ether, trimethylcyclohexanone, 1,1,1-tricholoroethane, hexane, hexanol, isobutyl acetate, butyl acetate, isophorone, nitropropane, butyl glycol acetate, 2-nitropropane, methylene chloride, methyl isobutyl ketone, cyclohexanone, isopropyl acetate, methylbenzyl alcohol, cyclohexanol, nitroethane, methyl tert-butyl ether, ethyl acetate, diethyl ether, butanol, butyl glycolate, isobutanol, 2-butanol, propylene carbonate, ethyl glycol acetate, methyl acetate, methyl ethyl ketone, or a combination thereof; or a combination thereof the forgoing. In other facets, the hydrocarbon comprises an aliphatic hydrocarbon, a cycloaliphatic hydrocarbon, a terpene, an aromatic hydrocarbon, or a combination thereof; wherein the oxygenated compound comprises an alcohol, an ester, a glycol ether, a ketone, an ether, or a combination thereof; or a combination thereof the forgoing. In particular facets, the hydrocarbon comprises a petroleum ether, pentane, hexane, heptane, isododecane, a kerosene, a mineral spirit, a VMP naphtha, cyclohexane, methylcyclohexane, ethylcyclohexane, tetrahydronaphthalene, decahydronaphthalene, wood terpentine oil, pine oil, α-pinene, β-pinene, dipentene, D-limonene, benzene, toluene, ethylbenzene, xylene, cumene, a type I high flash aromatic naphtha, a type II high flash aromatic naphtha, mesitylene, pseudocumene, cymol, styrene, or a combination thereof; wherein the oxygenated compound comprises methanol, ethanol, propanol, isopropanol, 1-butanol, isobutanol, 2-butanol, tert-butanol, amyl alcohol, isoamyl alcohol, hexanol, methylisobutylcarbinol, 2-ethylbutanol, isooctyl alcohol, 2-ethylhexanol, isodecanol, cylcohexanol, methylcyclohexanol, trimethylcyclohexanol, benzyl alcohol, methylbenzyl alcohol, furfuryl alcohol, tetrahydrofurfuryl alcohol, diacetone alcohol, trimethylcyclohexanol, methyl formate, ethyl formate, butyl formate, isobutyl formate, methyl acetate, ethyl acetate, propyl acetate, isopropyl acetate, butyl acetate, isobutyl acetate, sec-butyl acetate, amyl acetate, isoamyl acetate, hexyl acetate, cyclohexyl acetate, benzyl acetate, methyl glycol acetate, ethyl glycol acetate, butyl glycol acetate, ethyl diglycol acetate, butyl diglycol acetate, 1-methoxypropyl acetate, ethoxypropyl acetate, 3-methoxybutyl acetate, ethyl 3-ethoxypropionate, isobutyl isobutyrate, ethyl lactate, butyl lactate, butyl glycolate, dimethyl adipate, glutarate, succinate, ethylene carbonate, propylene carbonate, butyrolactone, methyl glycol, ethyl glycol, propyl glycol, isopropyl glycol, butyl glycol, methyl diglycol, ethyl diglycol, butyl diglycol, ethyl triglycol, butyl triglycol, diethylene glycol dimethyl ether, methoxypropanol, isobutoxypropanol, isobutyl glycol, propylene glycol monoethyl ether, 1-isopropoxy-2-propanol, propylene glycol mono-n-propyl ether, propylene glycol n-butyl ether, methyl dipropylene glycol, methoxybutanol, acetone, methyl ethyl ketone, methyl propyl ketone, methyl isopropyl ketone, methyl butyl ketone, methyl isobutyl ketone, methyl amyl ketone, methyl isoamyl ketone, diethyl ketone, ethyl amyl ketone, dipropyl ketone, diisopropyl ketone, cyclohexanone, methylcylcohexanone, trimethylcyclohexanone, mesityl oxide, diisobutyl ketone, isophorone, diethyl ether, diisopropyl ether, dibutyl ether, di-sec-butyl ether, methyl tert-butyl ether, tetrahydrofuran, 1,4-dioxane, metadioxane, or a combination thereof; wherein the chlorinated hydrocarbon comprises methylene chloride, trichloromethane, tetrachloromethane, ethyl chloride, isopropyl chloride, 1,2-dichloroethane, 1,1,1-trichloroethane, trichloroethylene, 1,1,2,2-tetrachlorethane, 1,2-dichloroethylene, perchloroethylene, 1,2-dichloropropane, chlorobenzene, or a combination thereof; wherein the nitrated hydrocarbon comprises a nitroparaffin, N-methyl-2-pyrrolidone, or a combination thereof; wherein the miscellaneous organic liquid comprises carbon dioxide; acetic acid, methylal, dimethylacetal, N,N-dimethylformamide, N,N-dimethylacetamide, dimethylsulfoxide, tetramethylene suflone, carbon disulfide, 2-nitropropane, N-methylpyrrolidone, hexamethylphosphoric triamide, 1,3-dimethyl-2-imidazolidinone, or a combination thereof; wherein the plasticizer comprises di(2-ethylhexyl) azelate; di(butyl) sebacate; di(2-ethylhexyl) phthalate; di(isononyl) phthalate; dibutyl phthalate; butyl benzyl phthalate; di(isooctyl) phthalate; di(idodecyl) phthalate; tris(2-ethylhexyl) trimellitate; tris(isononyl) trimellitate; di(2-ethylhexyl) adipate; di(isononyl) adipate; acetyl tri-n-butyl citrate; an epoxy modified soybean oil; 2-ethylhexyl epoxytallate; isodecyl diphenyl phosphate; tricresyl phosphate; isodecyl diphenyl phosphate; tri-2-ethylhexyl phosphate; an adipic acid polyester; an azelaic acid polyester; a bisphenoxyethylformal, or a combination thereof; or a combination thereof the forgoing.

In additional aspects, the colorant comprises a pigment, a dye, or a combination thereof. In particular facets, the active enzyme comprises a particulate material comprising about 0.000001% to about 100% of the pigment. In other facets, the pigment volume concentration of wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, is about 20% to about 70%. In additional facets, the pigment comprises a corrosion resistance pigment, a camouflage pigment, a color property pigment, an extender pigment, or a combination thereof. In further facets, the corrosion resistance pigment comprises aluminum flake, aluminum triphosphate, aluminum zinc phosphate, ammonium chromate, barium borosilicate, barium chromate, barium metaborate, basic calcium zinc molybdate, basic carbonate white lead, basic lead silicate, basic lead silicochromate, basic lead silicosulfate, basic zinc molybdate, basic zinc molybdate-phosphate, basic zinc molybdenum phosphate, basic zinc phosphate hydrate, bronze flake, calcium barium phosphosilicate, calcium borosilicate, calcium chromate, calcium plumbate, calcium strontium phosphosilicate, calcium strontium zinc phosphosilicate, dibasic lead phosphite, lead chromosilicate, lead cyanamide, lead suboxide, lead sulfate, mica, micaceous iron oxide, red lead, steel flake, strontium borosilicate, strontium chromate, tribasic lead phophosilicate, zinc borate, zinc borosilicate, zinc chromate, zinc dust, zinc hydroxy phosphite, zinc molybdate, zinc oxide, zinc phosphate, zinc potassium chromate, zinc silicophosphate hydrate, zinc tetraoxylchromate, or a combination thereof; wherein the camouflage pigment comprises an anthraquinone black, a chromium oxide green, the active enzyme comprising a particulate material, or a combination thereof; wherein the color property pigment comprises a black pigment, a brown pigment, a white pigment, a pearlescent pigment, a violet pigment, a blue pigment, a green pigment, a yellow pigment, an orange pigment, a red pigment, a metallic pigment, the active enzyme comprising a particulate material, or a combination thereof; wherein the extender pigment comprises a barium sulphate, a calcium carbonate, a kaolin, a calcium sulphate, a silicate, a silica, an alumina trihydrate, an active enzyme comprising a particulate material, or a combination thereof; or a combination thereof the forgoing. In particular facets, the color property pigment comprises aniline black; anthraquinone black; carbon black; copper carbonate; graphite; iron oxide; micaceous iron oxide; manganese dioxide, azo condensation, metal complex brown; antimony oxide; basic lead carbonate; lithopone; titanium dioxide; white lead; zinc oxide; zinc sulphide; titanium dioxide and ferric oxide covered mica, bismuth oxychloride crystal, dioxazine violet, carbazole Blue; cobalt blue; indanthrone; phthalocyanine blue; Prussian blue; ultramarine; chrome green; hydrated chromium oxide; phthalocyanine green; anthrapyrimidine; arylamide yellow; barium chromate; benzimidazolone yellow; bismuth vanadate; cadmium sulfide yellow; complex inorganic color; diarylide yellow; disazo condensation; flavanthrone; isoindoline; isoindolinone; lead chromate; nickel azo yellow; organic metal complex; yellow iron oxide; zinc chromate; perinone orange; pyrazolone orange; anthraquinone; benzimidazolone; BON arylamide; cadmium red; cadmium selenide; chrome red; dibromanthrone; diketopyrrolo-pyrrole; lead molybdate; perylene; pyranthrone; quinacridone; quinophthalone; red iron oxide; red lead; toluidine red; tonor; β-naphthol red; aluminum flake; aluminum non-leafing, gold bronze flake, zinc dust, stainless steel flake, nickel flake, nickel powder, barium ferrite; borosilicate; burnt sienna; burnt umber; calcium ferrite; cerium; chrome orange; chrome yellow; chromium phosphate; cobalt-containing iron oxide; fast chrome green; gold bronze powder; luminescent; magnetic; molybdate orange; molybdate red; oxazine; oxysulfide; polycyclic; raw sienna; surface modified pigment; thiazine; thioindigo; transparent cobalt blue; transparent cobalt green; transparent iron blue; transparent zinc oxide; triarylcarbonium; zinc cyanamide; zinc ferrite; or a combination thereof.

In some aspects, the additive comprises 0.000001% to 20.0% by weight, of the architectural coating, the automotive coating, the can coating, the sealant coating, the chemical agent resistant coating, the camouflage coating, the pipeline coating, the traffic marker coating, the aircraft coating, the nuclear power plant coating, or a combination thereof. In other aspects, the additive comprises an accelerator, an adhesion promoter, an antifoamer, anti-insect additive, an antioxidant, an antiskinning agent, a buffer, a catalyst, a coalescing agent, a corrosion inhibitor, a defoamer, a dehydrator, a dispersant, a drier, electrical additive, an emulsifier, a filler, a flame/fire retardant, a flatting agent, a flow control agent, a gloss aid, a leveling agent, a marproofing agent, a preservative, a silicone additive, a slip agent, a surfactant, a light stabilizer, a rheological control agent, a wetting additive, a cryopreservative, a xeroprotectant, a pH indicator, or a combination thereof. In some facets, the preservative comprises an in-can preservative, an in-film preservative, or a combination thereof. In additional facets, the preservative comprises a biocide, a biostatic, or a combination thereof. In particular facets, the biocide, the biostatic, or a combination thereof comprises an algaecide, an algaestatic, a bactericide, a bacteristatic, a fungicide, a fungistatic, a germicide, a germistatic, a herbicide, a herbistatic, a microbiocide, a microbiostatic, a mildewcide, a mildewstatic, a molluskicide, a molluskistatic, a slimicide, a slimistatic, a viricide, a viristatic, or a combination thereof. In additional facets, the preservative comprises 1-(3-chloroallyl)-3,5,7-triaza-1-azoniaadamantane chloride; 1,2-benzisothiazoline-3-one; 1,2-dibromo-2,4-dicyanobutane; 1,3-bis(hydroxymethyl)-5,5-dimethylhydantoin; 1-methyl-3,5,7-triaza-1-azonia-adamantane chloride; 2-bromo-2-nitropropane-1,3-diol; 2-(4-thiazolyl)benzimidazole; 2-(hydroxymethyl)-amino-2-methyl-1-propanol; 2(hydroxymethyl)-aminoethanol; 2,2-dibromo-3-nitrilopropionamide; 2,4,5,6-tetrachloro-isophthalonitrile; 2-mercaptobenzo-thiazole; 2-methyl-4-isothiazolin-3-one; 2-n-octyl-4-isothiazoline-3-one; 3-iodo-2-propynl N-butyl carbamate; 4,5-dichloro-2-N-octyl-3(2H)-isothiazolone; 4,4-dimethyloxazolidine; 5-chloro-2-methyl-4-isothiazolin-3-one; 5-hydroxy-methyl-1-aza-3,7-dioxabicylco (3.3.0.)octane; 6-acetoxy-2,4-dimethyl-1,3-dioxane; 7-ethyl bicyclooxazolidine; a combination of 1,2-benzisothiazoline-3-one and hexahydro-1,3,5-tris(2-hydroxyethyl)-s-triazine; a combination of 1,2-benzisothiazoline-3-one and zinc pyrithione; a combination of 2-(thiocyanomethyl-thio)benzothiozole and methylene bis(thiocyanate); a combination of 4-(2-nitrobutyl)-morpholine and 4,4′-(2-ethylnitrotrimethylene)dimorpholine; a combination of 4,4-dimethyl-oxazolidine and 3,4,4-trimethyloxazolidine; a combination of 5-chloro-2-methyl-4-isothiazolin-3-one and 2-methyl-4-isothiazolin-3-one; a combination of carbendazim and 3-iodo-2-propynl N-butyl carbamate; a combination of carbendazim, 3-iodo-2-propynl N-butyl carbamate and diuron; a combination of chlorothalonil and 3-iodo-2-propynl N-butyl carbamate; a combination of chlorothalonil and a triazine compound; a combination of tributyltin benzoate and alkylamine hydrochlorides; a combination of zinc-dimethyldithiocarbamate and zinc 2-mercaptobenzothiazole; a copper soap; a metal soap; a mercury soap; a mixture of bicyclic oxazolidines; a tin soap; an alkylamine hydrochloride; an amine reaction product; barium metaborate; butyl parahydroxybenzoate; carbendazim; copper(II) 8-quinolinolate; diiodomethyl-p-tolysulfone; dithio-2,2-bis(benzmethylamide); diuron; ethyl parahydroxybenzoate; glutaraldehyde; hexahydro-1,3,5-triethyl-s-triazine; hexahydro-1,3,5-tris(2-hydroxyethyl)-s-triazine; hydroxymethyl-5,5-dimethylhydantoin; methyl parahydroxybenzoate; N-butyl-1,2-benzisothiazolin-3-one; N-(trichloromethylthio) phthalimide; N-cyclopropyl-N-(1-dimethylethyl)-6-(methylthio)-1,3,5-triazine-2,4-diamine; N-trichloromethyl-thio-4-cyclohexene-1,2-dicarboximide; p-chloro-m-cresol; phenoxyethanol; phenylmercuric acetate; poly(hexamethylene biguanide) hydrochloride; potassium dimethyldithiocarbamate; potassium N-hydroxy-methyl-N-methyl-dithiocarbamate; propyl parahydroxybenzoate; sodium 2-pyridinethiol-1-oxide; tetra-hydro-3,5-di-methyl-2H-1,3,5-thiadiazine-2-thione; tributyltin benzoate; tributyltin oxide; tributyltin salicylate; zinc pyrithione; sodium pyrithione; copper pyrithione; zinc oxide; a zinc soap; or a combination thereof.

In certain embodiments, the elastomer comprises a thermoplastic elastomer, a melt processable rubber, a synthetic rubber, a natural rubber, a propylene oxide elastomer, an ethylene-isoprene elastomer, an ethylene-vinyl acetate elastomer, a non-polymeric elastomer, or a combination thereof. In some aspects, the thermoplastic elastomer comprises an elastomeric polyolefin, a thermoplastic vulcanizate, a styrenic thermoplastic elastomer, a styrene butadiene rubber, a polyurethane elastomer, a thermoplastic copolyester elastomer, a polyamide, or a combination thereof; wherein the synthetic rubber comprises a nitrile butadiene rubber, a butadiene rubber, a butyl rubber, a chlorinated/chlorosulfonated polyethylene, an epichlorohydrin, an ethylene propylene copolymer, a fluoroelastomer, a polyacrylate rubber, a poly(ethylene acrylic), a polychloroprene, a polyisoprene, a polysulfide rubber, a silicone rubber, or a combination thereof; wherein the non-polymeric elastomer comprises a vulcanized oil; or a combination thereof.

In some embodiments, the composition comprises an adhesive, a sealant, or a combination thereof. In other embodiments, the adhesive, the sealant, or a combination thereof; comprises an acrylic adhesive, an acrylic acid diester adhesive, a butyl rubber adhesive, a carbohydrate adhesive, a cellulosic adhesive, a cyanoacrylate adhesive, a cyanate ester adhesive, an epoxy adhesive, a melamine formaldehyde adhesive, a natural rubber adhesive, a neoprene rubber adhesive, a nitrile rubber adhesive, a phenolic adhesive, a phenoxy adhesive, a polyamide adhesive, a polybenzimidazole adhesive, a polyethylene adhesive, a polyester adhesive, a polyisobutylene adhesive, a polysulfide adhesive, a polyurethane adhesive, a polyvinyl acetal adhesive, a polyvinyl acetate adhesive, a polyvinyl alcohol adhesive, a protein adhesive, a reclaimed rubber adhesive, a resorcinol adhesive, a silicone adhesive, a styrene butadiene adhesive, an urea formaldehyde adhesive, a vinyl vinylidene adhesive, a non-polymeric adhesive, or a combination thereof. In particular facets, the non-polymeric adhesive comprises a mucilage adhesive.

In other embodiments, the elastomer; the adhesive; the sealant, or a combination thereof, comprises a polymeric material additive. In some aspects, the polymeric material additive comprises a curing agent, a cross-linking agent, an inhibitor, a nucleating agent, a plasticizer, a lubricant, a mold release agent, a slip agent, a diluent, a dispersant, a thickening agent, a thixotropic, a thinner, an anti-blocking agent, an antistatic agent, a flame retarder, a colorant, an antifogging agent, an odorant, a blowing agent, a surfactant, a defoamer, an anti-aging additive, a degrading agent, an anti-microbial agent, an adhesion promoter, an impact modifier, a low-profile additive, a filler, a pH indicator, or a combination thereof. In certain facets, the anti-microbial agent comprises a biocide, a biostatic, or a combination thereof.

In particular embodiments, the antibiological peptidic agent, the antibiological enzyme, or a combination thereof comprises a biocide, a biostatic, or a combination thereof.

In some embodiments, the composition is stored in a multi-pack container. In certain facets, about 0.000001% to about 100% of the active enzyme, the antibiological agent, or a combination thereof, is stored in a container of the multi-pack composition, and at least one composition component is stored in another container of the multi-pack.

Provided is a coating composition, comprising an architectural coating comprising an active enzyme, an antibiological peptidic agent, or a combination thereof, wherein the active enzyme comprises an esterase, a petroleum lipolytic enzyme, a ceramidase, a peptidase, an antibiological enzyme, or a combination thereof.

Provided is a multi-pack coating composition, comprising a plurality of containers, wherein at least one container comprises an active enzyme, an antibiological peptidic agent, or a combination thereof; wherein the active enzyme comprises an esterase, a petroleum lipolytic enzyme, a ceramidase, a peptidase, an antibiological enzyme, or a combination thereof; and wherein the coating comprises an architectural wood coating, an architectural masonry coating, an architectural artist coating, an automotive coating, a can coating, a sealant coating, a chemical agent resistant coating, a camouflage coating, a pipeline coating, a traffic marker coating, an aircraft coating, a nuclear power plant coating, or a combination thereof.

Provided is an elastomer composition, comprising an elastomer and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

Provided is a filler composition, comprising a filler and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

Provided is an adhesive composition, comprising an adhesive and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

Provided is a sealant composition, comprising a sealant and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

Provided is a textile finish composition, comprising a textile finish and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

Provided is a wax composition, comprising a wax and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

Provided is a method of preparing a bioactive surface treatment, a bioactive filler, or a combination thereof, comprising the steps of: obtaining an active enzyme, an antibiological peptidic agent, or a combination thereof; wherein the active enzyme comprises an esterase, a petroleum lipolytic enzyme, a ceramidase, a peptidase, an antibiological enzyme, or a combination thereof; and admixing at least one component of a surface treatment, a filler, or a combination thereof, with the active enzyme, the antibiological peptidic agent, or a combination thereof; and then admixing any additional component of a surface treatment, a filler, or a combination thereof to complete the surface treatment, the filler, or a combination thereof.

Provided is a method of preparing a bioactive surface treatment, a bioactive filler, or a combination thereof, comprising the steps of: obtaining an active enzyme, an antibiological peptidic agent, or a combination thereof; wherein the active enzyme comprises an esterase, a petroleum lipolytic enzyme, a ceramidase, a peptidase, an antibiological enzyme, or a combination thereof; and admixing a surface treatment, a filler, or a combination thereof, with the active enzyme, the antibiological peptidic agent, or a combination thereof.

Provided is a method of reducing the concentration of a chemical on a surface, comprising the steps of: applying a surface treatment to the surface, wherein the surface treatment comprises an active enzyme, and wherein the active enzyme comprises an esterase, a petroleum lipolytic enzyme, a ceramidase, a peptidase, an antibiological enzyme, or a combination thereof; and contacting the surface with a chemical, wherein the chemical comprises a substrate of the active enzyme; and wherein the substrate comprises an ester linkage, a peptide linkage, a lipid, a cell wall component, a cell membrane component, or a combination thereof. In some embodiments, the surface treatment comprises an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, or a combination thereof. In other embodiments the substrate is a component of a living cell, a virus, or a combination thereof, and wherein the active enzyme produces a biocidel activity, a biostatic activity, or a combination thereof upon contact with the substrate.

Provided is a method of cleaning a surface contaminated with a chemical, comprising the steps of: contacting a surface contaminated with a chemical with a coating comprising an active enzyme, wherein the active enzyme comprises an esterase, a petroleum lipolytic enzyme, a ceramidase, a peptidase, an antibiological enzyme, or a combination thereof, wherein the chemical comprises a substrate of the active enzyme; and wherein the substrate comprises an ester linkage, a peptide linkage, a lipid, a cell wall component, a cell membrane component, or a combination thereof.

Provided is a method of reducing the concentration of a chemical on a surface, comprising the steps of: applying a coating to the surface, wherein the coating comprises an architectural wood coating, an architectural masonry coating, an architectural artist coating, an automotive coating, a can coating, a sealant coating, a camouflage coating, a pipeline coating, a traffic marker coating, an aircraft coating, a nuclear power plant coating, or a combination thereof, and wherein the coating comprises an active enzyme, wherein the active enzyme comprises an esterase, a petroleum lipolytic enzyme, a ceramidase, a peptidase, an antibiological enzyme, or a combination thereof and contacting the surface with a chemical, wherein the chemical comprises a substrate of the active enzyme; and wherein the chemical comprises an ester linkage, a peptide linkage, a lipid, a cell wall component, a cell membrane component, or a combination thereof. In some embodiments, the step of applying to the surface a coating occurs prior to contacting the surface with the chemical. In certain embodiments, the surface is located on a stove, a sink, a drain pipe, a counter top, a floor, a wall, a cabinet, an appliance, or a combination thereof. In other aspects, the coating is formulated as an interior coating. In some embodiments, the method further comprises the step of: applying a cleaning material to the surface, and removing the chemical, a product of the reaction of the chemical catalyzed by the active enzyme, or a combination thereof. In particular aspects, the cleaning material comprises a cleaning solution, a cleaning devise, or a combination thereof.

Provided is a method of cleaning a surface contaminated with a chemical, comprising the steps of: obtaining a surface treatment comprising an active enzyme; and contacting a surface contaminated with a chemical with the surface treatment comprising an active enzyme, wherein the active enzyme comprises an esterase, a petroleum lipolytic enzyme, a ceramidase, a peptidase, an antibiological enzyme, or a combination thereof, wherein the chemical comprises a substrate of the active enzyme; and wherein the chemical comprises an ester linkage, a peptide linkage, a lipid, a cell wall component, a cell membrane component, or a combination thereof.

Provided is kit having component parts capable of being assembled comprising a container comprising an active enzyme, an antibiological peptidic agent, or a combination thereof, and a container comprising at least one component of an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; wherein the active enzyme comprises an esterase, a petroleum lipolytic enzyme, a ceramidase, a peptidase, an antibiological enzyme, or a combination thereof.

Provided is an article of manufacture, comprising an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; wherein the article of manufacture comprises an active enzyme, an antibiological peptidic agent, or a combination thereof, and wherein the active enzyme comprises an esterase, a petroleum lipolytic enzyme, a ceramidase, a peptidase, an antibiological enzyme, or a combination thereof.

Product is a composition. Product is a surface treatment. Product is a composition comprising a surface treatment. Product is a composition, comprising an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; comprising an active enzyme, an antibiological peptidic agent, or a combination thereof. Product a composition, obtainable by process of incorporation of an active enzyme, an antibiological peptidic agent, or a combination thereof; into an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof. Product a composition, comprising an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; comprising an active enzyme, an antibiological peptidic agent, or a combination thereof; for use as a medicament. Use of a compound an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; comprising an active enzyme, an antibiological peptidic agent, or a combination thereof; for the manufacture of a medicament for the treatment of a disease, the disease being a skin contamination with a chemical. A method for manufacturing product an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; comprising the steps of incorporating an active enzyme, an antibiological peptidic agent, or a combination thereof; into the architectural coating, the automotive coating, the can coating, the sealant coating, the chemical agent resistant coating, the camouflage coating, the pipeline coating, the traffic marker coating, the aircraft coating, the nuclear power plant coating; the elastomer; the adhesive; the sealant, the wax, the textile finish, the filler, or the combination thereof. Provided is an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; characterized in that an active enzyme, an antibiological peptidic agent, or a combination thereof; is included as a component of the architectural coating, the automotive coating, the can coating, the sealant coating, the chemical agent resistant coating, the camouflage coating, the pipeline coating, the traffic marker coating, the aircraft coating, the nuclear power plant coating; the elastomer; the adhesive; the sealant, the wax, the textile finish, the filler, or the combination thereof. Use of an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; for the purpose of reducing the concentration of a chemical on a surface.

### DETAILED DESCRIPTION OF THE EMBODIMENTS

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent therein. It is to be understood, however, that the present invention may be embodied in various forms. Therefore, specific details disclosed herein are not to be interpreted as limiting, but rather as a basis for the claims and as a representative basis for teaching to employ the present invention in virtually any appropriately detailed system, structure or manner. Other features will be readily apparent from the following detailed description; specific examples and claims; and various changes, substitutions, other uses and modifications that may be made to the embodiments disclosed herein without departing from the scope and spirit of the invention or as defined by the scope of the appended claims.

It should be understood that the biomolecular compositions, material formulations, surface treatments, fillers, materials, compounds, methods, procedures, and techniques described herein are presently representative of various embodiments. These techniques are intended to be exemplary, are given by way of illustration only, and are not intended as limitations on the scope. All patents and publications mentioned in this specification are herein incorporated by reference to the same extent as if each individual publication was specifically and individually indicated to be incorporated by reference.

As used herein other than the claims, the terms “a,” “an,” “the,” and/or “said” means one or more. As used herein in the claim(s), when used in conjunction with the words “comprise,” “comprises” and/or “comprising,” the words “a,” “an,” “the,” and/or “said” may mean one or more than one. As used herein and in the claims, the terms “having,” “has,” “is,” “have,” “including,” “includes,” and/or “include” has the same meaning as “comprising,” “comprises,” and “comprise.” As used herein and in the claims “another” may mean at least a second or more. As used herein and in the claims, “about” refers to any inherent measurement error or a rounding of digits for a value (e.g., a measured value, calculated value such as a ratio), and thus the term “about” may be used with any value and/or range.

The phrase “a combination thereof” “a mixture thereof” and such like following a listing, the use of “and/or” as part of a listing, a listing in a table, the use of “etc” as part of a listing, the phrase “such as,” and/or a listing within brackets with “e.g.,” or i.e., refers to any combination (e.g., any sub-set) of a set of listed components, and combinations and/or mixtures of related species and/or embodiments described herein though not directly placed in such a listing are also contemplated. For example, compositions described as a coating suitable for use on a plastic surface described in different sections of the specification may be claimed individually and/or as a combination, as they are part of the same genera of plastic coatings. In another example, various monomers of a chemical type such as “amino acid” may be described in various parts of the specification, and such amino acid monomers may be claimed individually and/or in various combinations. Such related and/or like genera(s), sub-genera(s), specie(s), and/or embodiment(s) described herein are contemplated both in the form of an individual component that may be claimed, as well as a mixture and/or a combination that may be described in the claims as “at least one selected from,” “a mixture thereof” and/or “a combination thereof.”

In various embodiments described herein, exemplary values are specified as a range, and all intermediate range(s), subrange(s), combination(s) of range(s) and individual value(s) within a cited range are contemplated and included herein. For example, citation of a range “0.03% to 0.07%” provides specific values within the cited range, such as, for example, 0.03%, 0.04%, 0.05%, 0.06%, and 0.07%, as well as various combinations of such specific values, such as, for example, 0.03%, 0.06% and 0.07%, 0.04% and 0.06%, and/or 0.05% and 0.07%, as well as sub-ranges such as 0.03% to 0.05%, 0.04% to 0.07%, and/or 0.04% to 0.06%, etc. Example 15 provides additional descriptions of specific numeric values within any cited range that may be used for an integer, intermediate range(s), subrange(s), combinations of range(s) and individual value(s) within a cited range, including in the claims.

In some embodiments, the average weight per single particle (“primary particle”) of a biomolecular composition (e.g., a cell-based particulate material) may be measured in “wet weight,” which refers to the weight of the particle prior to a drying and/or an extraction step that removes the liquid component of a biological cell (e.g., the aqueous component of the cell's cytoplasm). In certain aspects, the “wet weight” of a biomolecular composition (e.g., a whole cell particulate material) that has its liquid component replaced by some other liquid (e.g., an organic solvent) may also be measured in “wet weight.” The “dry weight” refers to the average per particle weight of a biomolecular composition after the majority of the liquid component has been removed. The term “majority” refers to about 50% to about 100%, with, for example, the greater values (e.g., about 85% to about 100%) contemplated in some aspects. In general embodiments, the dry weight of a biomolecular composition may be about 5% to about 30% the wet weight, as a cell often may comprise about 70% to about 95% water. Any technique for measuring a biological cell's and/or a particle's size, volume, density, etc. used for various insoluble particulate materials (e.g., a pigment, an extender) that typically are comprised as a component of a material formulation may be applied to a biomolecular composition to determine a wet weight value, a dry weight value, a particle size, and/or a particle density, etc. Various examples of specific techniques are described herein. Further, such measurements of a cell's size, shape, density, numbers, etc. are used in the art of microbiology, and may be applied herein with the embodiments. For example, the average number of particles, size, shape, etc. of a biomolecular composition may be microscopically determined for a given volume and/or weight of a material, whether prepared as a “wet weight” and/or a “dry weight material,” and the average particle weight, density, volume, etc. calculated. In some aspects, the average wet molecular weight or dry molecular weight of a primary particle of a biomolecular composition (e.g., a cell-based particulate material) comprises about 50 kDa to about 1.5×10¹⁴ kDa. The average active enzyme content, average antibiological peptidic agent content, or a combination thereof, per primary particle and/or per the content of the material formulation may comprise about 0.00000001% to about 100%.

Many variations of nomenclature are commonly used to refer to a specific chemical composition. Several common alternative names may be provided herein in quotations and/or parentheses/brackets, and/or other grammatical technique, adjacent to a chemical composition's designation when referred to herein. Many chemical compositions referred to herein are further identified by a Chemical Abstracts Service registration number. The Chemical Abstracts Service provides a unique numeric designation, denoted herein as “CAS No.,” for specific chemicals and some chemical mixtures, which unambiguously identifies a chemical composition's molecular structure.

In certain embodiments, the compositions and methods herein may produce materials (“material formulations”) (e.g., compositions, manufactured articles, etc) with a bioactivity. The disclosures herein describe various embodiments where a biomolecule's activity (e.g., an enzyme's catalytic reaction, a peptide's antimicrobial activity) may be conferred to a material via incorporation of a biomolecule into and/or upon the surface of the material to maintain a property, alter a property, and/or confer a property to the material. Examples of such a material formulation include a surface treatment, a filler, a biomolecular composition, or a combination thereof. Examples of a property that may be altered include resistance to a microorganism; while examples of a property that may be conferred include enzymatic activity upon contact with a substrate (e.g., a lipid, an organophosphorus compound, etc.) of an enzyme, wherein the material comprises the enzyme. Numberous examples of component(s), material formulation(s), composition(s), manufactured article(s), etc. are described herein, and inclusion of a biomolecular composition may alter and/or confer a property that to modify such component(s), material formulation(s), composition(s), manufactured article(s), etc. to be useable for a different purpose and/or function. In an example, a lipolytic enzyme may confer a self-degreasing property to a material formulation. In another example, a proteinaceous composition (e.g., a peptide composition, an enzyme) possessing an antibiological activity may be incorporated into a material formulation to alter and/or confer a property (e.g., an antibiological activity, a sufficient antifungal activity) that may be exhibited in the material formulation.

An example of a material formulation comprises a “surface treatment,” which refers to a composition applied to a surface, and examples of such compositions specifically contemplated include a coating (e.g., a paint, a clear coat), a textile finish, a wax, an elastomer, an adhesive, a filler, and/or a sealant. In some embodiments, such a surface treatment may be prepared as an amorphous material (e.g., a liquid, a semisolid) and/or a simple geometric shape (e.g., a planar material) to allow ease of application to a surface. An adhesive refers to a composition capable of attachment to one or more surfaces (“substrates”) of one or more objects (“adherents”), wherein the composition comprises a solid or is capable of converting into the solid, wherein the solid is capable of holding a plurality of objects (“adherents”) together by attachment to the surface of the objects while withstanding a normal operating stress load placed upon the objects and the solid. For example, an adhesive (e.g., a glue, a cement, an adhesive paste) may be capable of uniting, bonding and/or holding at least two surfaces together, usually in a strong and permanent manner. A sealant comprises a composition capable of attachment to a plurality of surfaces to fill a space and/or a gap between the plurality of surfaces and form a barrier to a gas, a liquid, a solid particle, an insect, or a combination thereof. An adhesive generally functions to prevent movement of the adherents, while a sealant typically functions to seal adherents that move. A sealant comprises a subtype of an adhesive based on purpose/function (i.e., a flexible adhesive), and a sealant typically possesses lower strength, greater flexibility, or a combination thereof, than many other types of adhesives (e.g., a structural adhesive). In contrast to adhesive and/or a sealant, an adhesive comprises a material (e.g., a coating such as a clear coating or a paint; or a mold release agent such as a plastic release film) applied to a surface to inhibit adhesion/sticking of an additional material to the adhesive and/or a surface the adhesive covers.

An elastomer (“elastomeric material”) comprises a “macromolecular material that returns rapidly to approximately the initial dimensions and shape after substantial deformation by a weak stress and release of the stress” while a rubber comprises a material “capable of recovering from a large deformation quickly and forcibly, and can be, and/or are already is, modified to a state in which it is essentially insoluble (but can swell) in a solvent.” Examples of a solvent commonly used to swell a rubber include benzene, methyl ethyl ketone, and/or ethanol toluene azeotrope (see, for example, definitions in ASTM D 1566). A rubber retracts within about one minute to less than about 1.5 times its original length after being held for about one minute at about twice its length at room temperature, while an elastomer retracts within about five minutes to within about 10% original length after being held for about five minutes at about twice its length at room temperature. Often cross-linking/vulcanization may be used to confer an elastomeric property, as the cross-links promote maintenance of a material's dimensions. A plastic comprises a solid polymeric material solid at room temperature (i.e., about 23° C.) in a finished state, and at some stage of the plastic's manufacture and/or processing was capable of being shaped by flow and/or molding into a finished article. A material such as an elastomer, a textile, an adhesive, or a paint, which may in some cases meet this definition, are not considered to be a plastic. All plastics comprise a polymer, but not all polymers are a plastic, such as, for example, a cellulose that lacks a chemical modification to allow it to be processed as a plastic during manufacture, or a polymer that possesses an elastomeric property. All polymeric materials comprise a polymer, but not all polymers possess the physical/chemical properties to be classified as a specific material type, particularly when such a material type comprises another component in addition to the polymer.

Further, some terms often have different meanings for different material types and/or uses being described, and the meaning applicable to the material should be applied as appropriate in the context, as understood in the applicable art. For example, a “cell” in a biotechnology art described for production of a biomolecule refers to the smallest unit of living matter (viruses not withstanding), while a “cell” in a material art (e.g., an elastomer art) refers to a void in a material to produce a solid foam material (e.g., elastomer foam material). In another example, the word “mold” may be used in the context of a fungal cell, while in other context “mold” refers to a solid structure used to shape a material, such as a mold used to shape an elastomeric material into a geometric shape. In such instances, the appropriate definition and/or meaning for the term (e.g., a biomolecular composition produced from a cell vs a void, a solid foamed material vs. a liquid or gas foam; a biological cell/organism vs. a device for material manufacture) should be applied in accordance with the context of the term's use in light of the present disclosures.

### A. BIOMOLECULES

As used herein, a “biomolecular composition” or “biomolecule composition” refers to a composition comprising a biomolecule. As used herein, a “biomolecule” refers to a molecule (e.g., a compound) comprising of one or more chemical moiety(s) [“specie(s),” “group(s),” “functionality(s),” “functional group(s)”] typically synthesized in living organisms, including but not limited to, an amino acid, a nucleotide, a polysaccharide, a simple sugar, a lipid, or a combination thereof. Examples of a biomolecule includes, a colorant (e.g., a chlorophyll), an enzyme, an antibody, a receptor, a transport protein, structural protein, a prion, an antibiological proteinaceous molecule (e.g., an antimicrobial proteinaceous molecule, an antifungal proteinaceous molecule), or a combination thereof. A biomolecule typically comprises a proteinaceous molecule. As used herein a “proteinaceous molecule,” proteinaceous composition,” and/or “peptidic agent” comprises a polymer formed from an amino acid, such as a peptide (i.e., about 3 to about 100 amino acids), a polypeptide (i.e., about 101 or more amino acids, such as about 50,000 or more amino acids), and/or a protein. As used herein a “protein” comprises a proteinaceous molecule comprising a contiguous molecular sequence three amino acids or greater in length, matching the length of a biologically produced proteinaceous molecule encoded by the genome of an organism. Examples of a proteinaceous molecule include an enzyme, an antibody, a receptor, a transport protein, a structural protein, or a combination thereof. Examples of a peptide (e.g., an inhibitory peptide, an antifungal peptide) of about 3 to about 100 amino acids (e.g., about 3 to about 15 amino acids). A peptidic agent and/or proteinaceous molecule may comprise a mixture of such peptide(s) (e.g., an aliquot of a peptide library), polypeptide(s) and/or protein(s), and may also include materials such as any associated stabilizer(s), carrier(s), and/or inactive peptide(s), polypeptide(s), and/or protein(s).

In some embodiments, a proteinaceous molecule comprises an enzyme. A proteinaceous molecule that functions as an enzyme, whether identical to the wild-type amino acid sequence encoded by an isolated gene, a functional equivalent of such a sequence, or a combination thereof, may be used. As used herein, a “wild-type enzyme” refers to an amino acid sequence that functions as an enzyme and matches the sequence encoded by an isolated gene from a natural source. As used herein, a “functional equivalent” to the wild-type enzyme generally comprises a proteinaceous molecule comprising a sequence and/or a structural analog of a wild-type enzyme's sequence and/or structure and functions as an enzyme. The functional equivalent enzyme may possess similar or the same enzymatic properties, such as catalyzing chemical reactions of the wild-type enzyme's EC classification; and/or may possess other enzymatic properties, such as catalyzing the chemical reactions of an enzyme related to the wild-type enzyme by sequence and/or structure. An enzyme encompasses its functional equivalents that catalyze the reaction catalyzed by the wild-type form of the enzyme (e.g., the reaction used for EC Classification). For example, the term “lipase” encompasses any functional equivalent of a lipase (i.e., in the claims, “lipase” encompasses such functional equivalents, “human lipase” encompasses functional equivalents of a wild-type human lipase, etc.) that retains lipase activity (e.g., catalyzes the reaction: triacylglycerol+H₂O=diacylglycerol+a carboxylate), though the activity may be altered (e.g., increased reaction rates, decreased reaction rates, altered substrate preference, etc.). Examples of a functional equivalent of a wild-type enzyme are described herein, and include mutations to a wild-type enzyme sequence, such as a sequence truncation, an amino acid substitution, an amino acid modification, and/or a fusion protein, etc., wherein the altered sequence functions as an enzyme. As used herein, the term “derived” refers to a biomolecule's (e.g., an enzyme) progenitor source, though the biomolecule may comprise a wild-type and/or a functional equivalent of the original source biomolecule, and thus the term “derived” encompasses both wild-type and functional equivalents. For example, a coding sequence for a Homo sapiens enzyme may be mutated and recombinantly expressed in bacteria, and the bacteria comprising the enzyme processed into a biomolecular composition for use, but the enzyme, whether isolated and/or comprising other bacterial cellular material(s), comprises an enzyme “derived” from Homo sapiens. In another example, a wild-type enzyme isolated from an endogenous biological source, such as, for example, a Pseudomonas putida lipase isolated from Pseudomonas putida, comprises an enzyme “derived” from Pseudomonas putida. In some cases, a biomolecule may comprise a hybrid of various sequences, such as a fusion of a mammalian lipase and a non-mammalian lipase, and such a biomolecule may be considered derived from both sources. Other types of biomolecule(s) (e.g., a ribozyme, a transport protein, etc.) may be derived, isolated, produced, in a wild-type or a functional equivalent form. In other aspects, a biomolecule may be derived from a non-biological source, such as the case of a proteinaceous and/or a nucleotide sequence engineered by the hand of man. For example, a nucleotide sequence encoding a synthetic peptide sequence from a peptide library, such as SEQ ID Nos. 1 to 47, may be recombinantly produced, and may thus “derived” from the originating peptide library.

In some embodiments, a biomolecular composition comprises a cell and/or cell debris (i.e., a “cell-based” material), in contrast to a purified biomolecule (e.g., a purified enzyme). In general embodiments, a cell used in a cell-based particulate material comprises a durable structure at the cell-external environment interface, such as, for example, a cell wall, a silica based shell (“test”), a silica based exoskeleton (“frustule”), a pellicle, a proteinaceous outer coat, or a combination thereof. In typical embodiments, a cell may be obtained/isolated from a unicellular and/or an oligocellular organism, and a particulate material may be prepared from such an organism without a step to separate one or more cells from a multicellular tissue and/or a multicellular organism (e.g., a plant) into a smaller average particle size suitable for preparation of a material formulation (e.g., a biomolecular composition).

A biological material such as a virus (e.g., a bacteriophage), a biological cell (e.g., a microorganism), a virus, a tissue, and/or an organism (e.g., a plant) may be obtained from an environmental source using procedures of the art [see, for example, “Environmental Biotechnology Isolation of Biotechnological Organisms From Nature (Labeda, D. P., Ed.), 1990]. However, many live cultures, seeds, organisms, etc. of previously isolated and characterized biological materials have been conveniently cataloged and stored by public depositories and/or commercial vendors for the ease of use. Additionally, the identification of a biological material, particularly microorganisms, usually comprises characterization of suitable growth conditions for the cell and/or a virus, such as energy source (e.g., a digestible organic molecule), vitamin requirements, mineral requirements, pH conditions, light conditions, temperature, etc. [see, for example, “Bergey's Manual of Determinative Bacteriology Ninth Edition” (Hensyl, W. R., Ed.), 1994”; “The Yeasts—A Taxonomic Study—Fourth Revised and Enlarged Edition” (Kurtzman, C. P. and Fell, J. W., Eds.), 1998″; and “The Springer Index of Viruses” (Tidona, C. A. and Darai, G., Eds.), 2001]. Such biological materials and information about appropriate growth conditions may be obtainable from the biological culture collection and/or commercial vendor that stores the biological material. Hundreds of such biological culture collections currently exist, and the location of a specific biological material may be identified using a database such as that maintained by the World Data Center for Microorganisms (National Institute of Genetics, WFCC-MIRCEN World Data Center for Microorganisms, 1111 Yata, Mishima, Shizuoka, 411-8540 JAPAN). Specific examples of biological culture collections referred to herein include the American Type Culture Collection (“ATCC”; P.O. Box 1549, Manassas, Va. 20108-1549, U.S.A), the Culture Collection of Algae and Protozoa (“CCAP”; CEH Windermere, The Ferry House, Far Sawrey, Ambleside, Cumbria LA22 0LP, United Kingdom), the Collection de ('Institut Pasteur (“CIP”; Institut Pasteur, 28 Rue du Docteur Roux, 75724 Paris Cedex 15, France), the Deutsche Sammlung von Mikroorganismen and Zellkulturen (“DSMZ”; GmbH, Mascheroder Weg 1B, D-38124 Braunschweig, Germany), the HEM Biomedical Fungi and Yeasts Collection (“IHEM”; Scientific Institute of Public Health—Louis Pasteur, Mycology Section, Rue J. Wytsmanstraat 14, B-1050 Brussels), the Japan Collection of Microorganisms (“JCM”; Institute of Physical and Chemical Research (RIKEN), Wako, Saitama 351-0198, Japan), the Collection of the Laboratorium voor Microbiologie en Microbiele Genetica (“LMG”; Rijksuniversiteit, Ledeganckstraat 35, B-9000, Gent, Belgium), the MUCL (Agro)Industrial Fungi & Yeasts Collection (“MUCL,” Mycothèque de l′Universite catholique de Louvain, Place Croix du Sud 3, B-1348 Louvain-la-Neuve), the Pasteur Culture Collection of Cyanobacteria (“PCC”; Unité de Physiologie Microbienne, Institut Pasteur, 28 rue du Docteur Roux, 75724 Paris Cedex 15, France), the All-Russian Collection of Microorganisms (“VKM”; Russian Academy of Sciences, Institute of Biochemistry and Physiology of Microorganisms, 142292 Pushchino, Moscow Region, Russia), and the University of Texas (“UTEX”; Department of Botany, The University of Texas at Austin, Austin, Tex. 78713-7640).

As used herein, “unicellular” refers to 1 cell that generally does not live in contact with a second cell. As used herein, “oligocellular” refers to about 2 to about 100 cells, which generally live in contiguous contact with the other cells. Common specific types of oligocellular biological material includes 2 contacting cells (“dicellular”), three contacting cells (“tricellular”) and four contacting cells (“tetracellular”). As used herein, “multicellular” refers to 101 or more cells (e.g., hundreds, thousands, millions, billions, trillions), which generally live in contiguous contact with the other cells. In embodiments wherein the particulate cellular material primarily derives from a unicellular biological material (e.g., many microorganisms), the composition may be referred to herein as a “unicellular-based particulate material.” In embodiments wherein the particulate cellular material primarily derives from an oligocellular biological material (e.g., certain microorganisms, tissues), the composition may be known herein as an “oligocellular-based particulate material,” as well as a “dicellular-based particulate material,” tricellular-based particulate material,” or “tetracellular-based particulate material,” as appropriate. In embodiments wherein the cellular material primarily derives from a multicellular biological material (e.g., many eukaryotic organisms such as a visible plant), the composition may be known herein as a “multicellular-based particulate material.” A cell-based particulate material may be referred to herein based upon the type of biological material from which it was derived, including taxonomic/phylogenetic classification and/or biochemical composition, as well as one or more processing steps used in its preparation. Examples of such lexicography for a cell-based particulate material include an “eurkaryotic-based particulate material,” a “prokaryotic-based particulate material,” a “plant-based particulate material,” a “microorganism-based particulate material,” a “Eubacteria-based particulate material,” an “Archaea-based particulate material,” a “fungi-based particulate material,” a “yeast-based particulate material,” a “Protista-based particulate material,” an “algae-based particulate material,” a “Chrysophyta-based particulate material,” a “Methanolacinia-based particulate material,” a “Microscilla aggregans-based particulate material,” a “bacteriophage HER-6 [44Lindberg]-based particulate material,” a “bacteria and algae-based particulate material,” a “peptidoglycan-based particulate material,” a “pellicle-based particulate material,” an “attenuated viral-based particulate material,” a “sterilized microorganism-based particulate material,” an “encapsulated Streptomyces-based particulate material,” a “virus-based material,” etc.

Certain cell(s) and/or virus(s) are capable of growth in environmental conditions typically harmful to many other types of cells (“extremophiles”), such as conditions of extreme temperature, salt and/or pH. A biomolecule derived from such a cell and/or a virus may be useful in certain embodiments for durability, activity, or other property of a biomolecular composition (e.g., a material formulation comprising a biomolecular composition) that undergoes conditions similar to (e.g., the same or overlapping ranges) as those found in the cell's and/or the virus's growth environment. For example, a hyperthermophile-based biomolecular composition may find usefulness in a material formulation where high temperature thermal extremes may occur, including extremes of temperature that may occur during coating based film formation and/or use of a coating produced film near a heat source. For example, a “hyperthermophile” or “thermophile” typically grows in temperatures considered herein to comprise a baking temperature for a coating (e.g., greater than about 40° C., often up to about 120° C. or more), and some compositions may comprise a biomolecule derived from a thermophile. In other embodiments, a biomolecular composition with prolonged stability, enzymatic activity, or a combination thereof, at other temperature ranges may be used depending upon the application. As used herein, a “psychrophile” typically grows at about −10° C. to about 20° C., and a “mesophile” typically grows at about 20° C. to about 40° C., and may be used to obtain a biomolecular composition for an application in a temperature range within and/or overlapping those of a psychrophile and/or a mesophile (.e.g., ambient conditions). As used herein, an “extreme halophile” may be capable of living in salt-water conditions of about 1.5 M (8.77% w/v) sodium chloride to about 2.7 M (15.78% w/v) or more sodium chloride. An extreme halophile's biomolecule component(s) may be relatively resistant to an ionic-salt component of a material formulation. As used herein, an “extreme acidophile” may be capable of growing in about pH 1 to about pH 6, while an “extreme alkaliphile” may be capable of growing in about pH 8 to about pH 14. One or more biomolecules such as an enzyme derived from such a cell and/or a virus may be selected on the basis the cell's and/or a virus's growth conditions for incorporation into the compositions, articles, etc. described herein.

In addition to the sources described herein for a biomolecule, a reagent, a living cell, etc., such a material and/or a chemical formula thereof may be obtained from convenient source such as a public database, a biological depository, and/or a commercial vendor. For example, various nucleotide sequences, including those that encode amino acid sequences, may be obtained at a public database, such as the Entrez Nucleotides database, which includes sequences from other databases including GenBank (e.g., CoreNucleotide), RefSeq, and PDB. Another example of a public databank for nucleotide and amino acid sequences includes the Kyoto Encyclopedia of Genes and Genomes (“KEEG”) (Kanehisa, M. et al., 2008; Kanehisa, M. et al., 2006; Kanehisa, M. and Goto, S., 2000). In another example, various amino acid sequences may be obtained at a public database, such as the Entrez databank, which includes sequences from other databases including SwissProt, PIR, PRF, PDB, Gene, GenBank, and RefSeq. Numerous nucleic acid sequences and/or encoded amino acid sequences can be obtained from such sources. In a further example, a biological material comprising, or are capable of comprising such a biomolecule (e.g., a living cell, a virus), may be obtained from a depository such as the American Type Culture Collection (“ATCC”), P.O. Box 1549 Manassas, Va. 20108, USA. In an additional example, a biomolecule, a chemical reagent, a biological material, and/or an equipment may be obtained from a commercial vendor such as Amersham Biosciences®, 800 Centennial Avenue, P.O. Box 1327, Piscataway, N.J. 08855-1327 USX; BD Biosciences®, including Clontech®, Discovery Labware®, Immunocytometry Systems® and Pharmingen®, 1020 East Meadow Circle, Palo Alto, Calif. 94303-4230 USX; Invitrogen™, 1600 Faraday Avenue, PO Box 6482, Carlsbad, Calif. 92008 USX; New England Biolabs®, 32 Tozer Road, Beverly, Mass. 01915-5599 USX; Merck®, One Merck Drive, P.O. Box 100, Whitehouse Station, N.J. 08889-0100 USX; Novagene®, 441 Charmany Dr., Madison, Wis. 53719-1234 USX; Promega®, 2800 Woods Hollow Road, Madison Wis. 53711 USX; Pfizer®, including Pharmacia®, 235 East 42nd Street, New York, N.Y. 10017 USX; Quiagen®, 28159 Avenue Stanford, Valencia, Calif. 91355 USX; Sigma-Aldrich®, including Sigma, Aldrich, Fluka, Supelco and Sigma-Aldrich Fine Chemicals, PO Box 14508, Saint Louis, Mo. 63178 USX; Wako Pure Chemical Industries, Ltd, 1-2 Doshomachi 3-Chome, Chuo-ku, Osaka 540-8605, Japan; TCI America, 9211 N. Harborgate Street, Portland, Oreg. 97203, U.S.A.; Reactive Surfaces, Ltd, 300 West Avenue Step #1316, Austin, Tex. 78701; Stratagene®, 11011 N. Torrey Pines Road, La Jolla, Calif. 92037 USA, etc. In a further example, a biomolecule, a chemical reagent, a biological material, and/or an equipment may be obtained from commercial vendors such as Amersham Biosciences®, 800 Centennial Avenue, P.O. Box 1327, Piscataway, N.J. 08855-1327 USA”; Allen Bradley, 1201 South Second Street, Milwaukee, Wis. 53204-2496, USA”; BD Biosciences®, including Clontech®, Discovery Labware®, Immunocytometry Systems® and Pharmingen®, 1020 East Meadow Circle, Palo Alto, Calif. 94303-4230 USA”; Baker, Mallinckrodt Baker, Inc., 222 Red School Lane, Phillipsburg N.J. 08865, U.S.A.”; Bioexpression and Fermentation Facility, Life Sciences Building, 1057 Green Street, University of Georgia, Athens, Ga. 30602, USA”; Bioxpress Scientific, PO Box 4140, Mulgrave Victoria 3170”; Boehringer Ingelheim GmbH, Corporate Headquarters, Binger Str. 173, 55216 Ingelheim, Germany Chem Service, Inc, PO Box 599, West Chester, Pa. 19381-0599, USA”; Difco, Voigt Global Distribution Inc., P.O. Box 1130, Lawrence, K S 66044-8130, USA”; Fisher Scientific, 2000 Park Lane Drive, Pittsburgh, Pa. 15275, USA”; Invitrogen™, 1600 Faraday Avenue, PO Box 6482, Carlsbad, Calif. 92008 USA”; Ferro Pfanstiehl Laboratories, Inc., 1219 Glen Rock Avenue, Waukegan, Ill. 60085-0439, USA”; New England Biolabs, 32 Tozer Road, Beverly, Mass. 01915-5599 USA”; Merck®, One Merck Drive, P.O. Box 100, Whitehouse Station, N.J. 08889-0100 USA”; Novozymes North America Inc., PO BOX 576, 77 Perry Chapel Church Road, Franklinton N.C. 27525 United States; Millipore Corporate Headquarters, 290 Concord Rd., Billerica, Mass. 01821, USA”; Nalgene®Labware, Nalge Nunc International, International Department, 75 Panorama Creek Drive, Rochester, N.Y. 14625. U.S.A.”; New Brunswick Scientific Co., Inc., 44 Talmadge Road, Edison, N.J. 08817 USA”; Novagene®, 441 Charmany Dr., Madison, Wis. 53719-1234 USA”; NCSRT, Inc., 1000 Goodworth Drive, Apex, N.C. 27539, USA”; Promega®, 2800 Woods Hollow Road, Madison Wis. 53711 USA”; Pfizer®, including Pharmacia®, 235 East 42nd Street, New York, N.Y. 10017 USA”; Quiagen®, 28159 Avenue Stanford, Valencia, Calif. 91355 USA”; SciLog, Inc., 8845 South Greenview Drive, Suite 4, Middleton, Wis. 53562, USA”; Sigma-Aldrich®, including Sigma, Aldrich, Fluka, Supelco, and Sigma-Aldrich Fine Chemicals, PO Box 14508, Saint Louis”; USB Corporation, 26111 Miles Road, Cleveland, Ohio 44128, USA”; Sherwin Williams Company, 101 Prospect Ave., Cleveland, Ohio, USA”; Lightnin, 135 Mt. Read Blvd., Rochester, N.Y. 14611 U.S.A.”; Amano Enzyme, USA Co., Ltd. 2150 Point Boulevard Suite 100 Elgin, Ill. 60123 U.S.A.”; Novozymes North America Inc., 77 Perry Chapel Church Road, Franklinton, N.C. 27525, U.S.A.”; and W B Moore, Inc., 1049 Bushkill Drive, Easton, Pa. 18042.

In addition to those techniques specifically described herein, a cell, nucleic acid sequence, amino acid sequence, and the like, may be manipulated in light of the present disclosures, using standard techniques [see, for example, In “Molecular Cloning” (Sambrook, J., and Russell, D. W., Eds.) 3rd Edition, Cold Spring Harbor, N.Y.: Cold Spring Harbor Laboratory Press, 2001”; In “Current Protocols in Molecular Biology” (Chanda, V. B. Ed.) John Wiley & Sons, 2002”; In “Current Protocols in Nucleic Acid Chemistry” (Harkins, E. W. Ed.) John Wiley & Sons, 2002”; In “Current Protocols in Protein Science” (Taylor, G. Ed.) John Wiley & Sons, 2002”; In “Current Protocols in Cell Biology” (Morgan, K. Ed.) John Wiley & Sons, 2002”; In “Current Protocols in Pharmacology” (Taylor, G. Ed.) John Wiley & Sons, 2002”; In “Current Protocols in Cytometry” (Robinson, J. P. Ed.) John Wiley & Sons, 2002”; In “Current Protocols in Immunology” (Coico, R. Ed.) John Wiley & Sons, 2002].

### B. ENZYMES

In many embodiments, selection of a biomolecule for use depends on the property to be conferred to a composition, an article, etc. In specific embodiments, a biomolecule comprises an enzyme, to confer a property such as as enzymatic activity to a material formulation (e.g., a surface treatment, a filler, a biomolecular composition). As used herein, the term “enzyme” refers to a molecule that possesses the ability to accelerate a chemical reaction, and comprises one or more chemical moiety(s) typically synthesized in living organisms, including but not limited to, an amino acid, a nucleotide, a polysaccharide, a simple sugar, a lipid, or a combination thereof. Enzymes are identified by a numeric classification system [See, for example, IUBM B (1992) Enzyme Nomenclature: Recommendations (1992) of the Nomenclature Committee of the International Union of Biochemistry and Molecular Biology. (NC-ICBMB and Edwin C. Webb Eds.) Academic Press, San Diego, Calif.; Enzyme nomenclature. Recommendations 1992, 1994; Enzyme nomenclature. Recommendations 1992, 1995; Enzyme nomenclature. Recommendations 1992, 1996; Enzyme nomenclature. Recommendations 1992, 1997; Enzyme nomenclature. Recommendations 1992, 1999].

An enzyme may function in synthesis and/or degradation, a catabolic reaction and/or an anabolic reaction, and other types of reversible reactions. For example, an enzyme normally described as an esterase may function as an ester synthetase depending upon the concentration of the substrate(s) and/or the product(s), such as an excess of hydrolyzed esters, typically considered the product of an esterase reaction, relative to unhydrolyzed esters, typically considered the substrate of the esterase reaction. In another example, a lipase may function as a lipid synthetase due to a relative abundance of free fatty acid(s) and alcohol moiety(s) to catalyze the synthesis of a fatty acid ester. Any reaction that an enzyme may be capable of is contemplated, such as, for example, a transesterification, an interesterification, and/or an intraesterification, and the like, being conducted by an esterase. For example, an esterase may alter the odor and/or fragrance of a composition by degrading an odor causing chemical, such as those produced by a microorganism, as well as synthesize a fragrant compound, as odor or fragrant compounds often comprises an ester linkage.

In the context of a biomolecule, “active” or “bioactive” refers to the effect of biomolecule, such as conferring and/or altering a property of a material formulation. For example, a material formulation comprising an “active” or “bioactive” antibiological peptide refers to the material formulation possessing altered and/or conferred antibiological effect (e.g., a biocidel effect, a biostatic effect) on a living cell (e.g., a living organism, a fungal cell) and/or a virus relative to a like material formulation lacking a similar content of the antibiological peptide, when the context allows. In another example, as used herein, the term “bioactive” or “active” refers to the ability of an enzyme, in the context of an enzyme, to accelerate a chemical reaction differentiating such activity from a like ability of a composition, an article, a method, etc. that does not comprise an enzyme to accelerate a chemical reaction. For example, a surface treatment comprising lysozyme that displays lysozyme activity comprises an active enzyme (e.g., a lysozyme EC 3.2.1.17). In another example, a surface treatment comprising a lipolytic enzyme and a non-enzyme catalyst of a lipolytic reaction that demonstrates an improved lipolytic activity (e.g., a statistically difference in activity; an improvement in a property as scored, such as from “good” to “excellent”, by an assay; etc.) relative to a similar surface treatment lacking an active lipolytic enzyme. An “effective amount” refers to a concentration of component of a material formulation and/or the material formulation itself (e.g., an antifungal peptide, a biomolecular composition) capable of exerting a desired effect (e.g., an antifungal effect).

In certain embodiments, an enzyme may comprise a simple enzyme, a complex enzyme, or a combination thereof. As known herein, a “simple enzyme” comprises an enzyme wherein a chemical property of one or more moiety(s) found in its amino acid sequence produces enzymatic activity. As known herein, a “complex enzyme” comprises an enzyme whose catalytic activity functions when an apo-enzyme combines with a prosthetic group, a co-factor, or a combination thereof. An “apo-enzyme” comprises a proteinaceous molecule and may be relatively catalytically inactive without a prosthetic group and/or a co-factor. As known herein, a “prosthetic group” or “co-enzyme” comprises a non-proteinaceous molecule that may be attached to the apo-enzyme to produce a catalytically active complex enzyme. As known herein, a “holo-enzyme” comprises a complex enzyme comprising an apo-enzyme and a co-enzyme. As known herein, a “co-factor” comprises a molecule that acts in combination with the apo-enzyme to produce a catalytically active complex enzyme. In some aspects, a prosthetic group comprises one or more bound metal atoms, a vitamin derivative, or a combination thereof. Examples of a metal atom that may be used in a prosthetic group and/or a co-factor include Ca, Cd, Co, Cu, Fe, Mg, Mn, Ni, Zn, or a combination thereof. Usually the metal atom comprises an ion, such as Ca²⁺, Cd²⁺, Co²⁺, Cu²⁺, Fe²⁺, Mg²⁺, Mn²⁺, Ni²⁺, Zn²⁺, or a combination thereof. As known herein, a “metalloenzyme” comprises a complex enzyme comprising an apo-enzyme and a prosthetic group, wherein the prosthetic group comprises a metal atom. As known herein, a “metal activated enzyme” comprises a complex enzyme comprising an apo-enzyme and a co-factor, wherein the co-factor comprises a metal atom.

A chemical that is capable of binding and/or is bound by a biomolecule (e.g., a proteinaceous molecule) may be known herein as a “ligand.” As used herein, “bind” or “binding” refers to a physical contact between the biomolecule (e.g., a proteinaceous molecule) at a specific region of the biomolecule (e.g., a proteinaceous molecule) and the ligand in a reversible fashion. Examples of a binding interaction include such interactions as a ligand known as an “antigen” binding an antibody, a ligand binding a receptor, a ligand binding an enzyme, a ligand binding a peptide and/or a polypeptide, and the like. A portion of the biomolecule (e.g., a proteinaceous molecule) wherein ligand binding occurs may be known herein as a “binding site.” A ligand acted upon by an enzyme in an accelerated chemical reaction may be known herein as a “substrate.” A contact between the enzyme and a substrate in a fashion suitable for the accelerated chemical reaction to proceed may be known herein as “substrate binding.” A portion of the enzyme involved in the chemical interactions that contributed to the accelerated chemical reaction may be known herein as an “active site.”

A chemical that slows and/or prevents the enzyme from conducting the accelerated chemical reaction may be known herein as an “inhibitor.” A contact between the enzyme and the inhibitor in a fashion suitable for slowing and/or preventing the accelerated chemical reaction to proceed upon a target substrate may be known herein as “inhibitor binding.” In some embodiments, inhibitor binding occurs at a binding site, an active site, or a combination thereof. In some aspects, an inhibitor's binding occurs without the inhibitor undergoing the chemical reaction. In specific aspects, the inhibitor may also comprise a substrate such as in the case of an inhibitor that precludes and/or reduces the ability of the enzyme in catalyzing the chemical reaction of a target substrate for the period of time inhibitor binding occurs at an active site and/or a binding site. In other aspects, an inhibitor undergoes the chemical reaction at a slower rate relative to a target substrate.

In some embodiments, enzymes may be described by the classification system of The International Union of Biochemistry and Molecular Biology (“IUBMB”). The IUBMB classifies enzymes by the type of reaction catalyzed and enumerates a sub-class by a designated enzyme commission number (“EC”). Based on these broad categories, an enzyme may comprise an oxidoreductase (EC 1), a transferase (EC 2), a hydrolase (EC 3), a lyase (EC 4), an isomerase (EC 5), a ligase (EC 6), or a combination thereof. An enzyme may be able to catalyze multiple reactions, and thus have activities of multiple EC classifications.

Generally, the chemical reaction catalyzed by an enzyme alters a moiety of a substrate. As used herein, a “moiety,” “group,” and/or “species” in the context of the field of chemistry, refers to a chemical sub-structure that may be a part of a larger molecule. Examples of a moiety include an acid halide, an acid anhydride, an alcohol, an aldehyde, an alkane, an alkene, an alkyl halide, an alkyne, an amide, an amine, an arene, an aryl halide, a carboxylic acid, an ester, an ether, a ketone, a nitrile, a phenol, a sulfide, a sulfonic acid, a thiol, etc.

An oxidoreductase catalyzes an oxido-reduction of a substrate, wherein the substrate comprises either a hydrogen donor and/or an electron donor. An oxidoreductase may be classified by the substrate moiety of the donor and/or the acceptor. Examples of an oxidoreductase include an oxidoreductase that acts on a donor CH—OH moiety, (EC 1.1); a donor aldehyde or a donor oxo moiety, (EC 1.2); a donor CH—CH moiety, (EC 1.3); a donor CH—NH₂ moiety, (EC 1.4); a donor CH—NH moiety, (EC 1.5); a donor nicotinamide adenine dinucleotide (“NADH”) or a donor nicotinamide adenine dinucleotide phosphate (“NADPH”), (EC 1.6); a donor nitrogenous compound, (EC 1.7); a donor sulfur moiety, (EC 1.8); a donor heme moiety, (EC 1.9); a donor diphenol and/or a related moiety as donor, (EC 1.10); a peroxide as an acceptor, (EC 1.11); a donor hydrogen, (EC 1.12); a single donor with incorporation of molecular oxygen (“oxygenase”), (EC 1.13); a paired donor, with incorporation or reduction of molecular oxygen, (EC 1.14); a superoxide radical as an acceptor, (EC 1.15); an oxidoreductase that oxidises a metal ion, (EC 1.16); an oxidoreductase that acts on a donor CH₂ moiety, (EC 1.17); a donor iron-sulfur protein, (EC 1.18); a donor reduced flavodoxin, (EC 1.19); a donor phosphorus or donor arsenic moiety, (EC 1.20); an oxidoreductase that acts on an X—H and an Y—H to form an X—Y bond, (EC 1.21); as well as an other oxidoreductase, (EC 1.97); or a combination thereof.

A transferase catalyzes the transfer of a moiety from a donor compound to an acceptor compound. A transferase may be classified based on the chemical moiety transferred. Examples of a transferase include a transferase that catalyzes the transfer of an one-carbon moiety, (EC 2.1); an aldehyde and/or a ketonic moiety, (EC 2.2); an acyl moiety, (EC 2.3); a glycosyl moiety, (EC 2.4); an alkyl and/or an aryl moiety other than a methyl moiety, (EC 2.5); a nitrogenous moiety, (EC 2.6); a phosphorus-containing moiety, (EC 2.7); a sulfur-containing moiety, (EC 2.8); a selenium-containing moiety, (EC 2.9); or a combination thereof.

A hydrolase catalyzes the hydrolysis of a chemical bond. A hydrolase may be classified based on the chemical bond cleaved or the moiety released or transferred by the hydrolysis reaction. Examples of a hydrolase include a hydrolase that catalyzes the hydrolysis of an ester bond, (EC 3.1); a glycosyl released/transferred moiety, (EC 3.2); an ether bond, (EC 3.3); a peptide bond, (EC 3.4); a carbon-nitrogen bond, other than a peptide bond, (EC 3.5); an acid anhydride, (EC 3.6); a carbon-carbon bond, (EC 3.7); a halide bond, (EC 3.8); a phosphorus-nitrogen bond, (EC 3.9); a sulfur-nitrogen bond, (EC 3.10); a carbon-phosphorus bond, (EC 3.11); a sulfur-sulfur bond, (EC 3.12); a carbon-sulfur bond, (EC 3.13); or a combination thereof.

Examples of an esterase (EC 3.1) include a carboxylic ester hydrolase (EC 3.1.1); a thioester hydrolase (EC 3.1.2); a phosphoric monoester hydrolase (EC 3.1.3); a phosphoric diester hydrolase (EC 3.1.4); a triphosphoric monoester hydrolase (EC 3.1.5); a sulfuric ester hydrolase (EC 3.1.6); a diphosphoric monoester hydrolase (EC 3.1.7); a phosphoric triester hydrolase (EC 3.1.8); an exodeoxyribonuclease producing a 5′-phosphomonoester (EC 3.1.11); an exoribonuclease producing a 5′-phosphomonoester (EC 3.1.13); an exoribonuclease producing a 3′-phosphomonoester (EC 3.1.14); an exonuclease active with a ribonucleic acid and/or a deoxyribonucleic acid and producing a 5′-phosphomonoester (EC 3.1.15); an exonuclease active with a ribonucleic acid and/or a deoxyribonucleic acid and producing a 3′-phosphomonoester (EC 3.1.16); an endodeoxyribonuclease producing a 5′-phosphomonoester (EC 3.1.21); an endodeoxyribonuclease producing a 3′-phosphomonoester (EC 3.1.22); a site-specific endodeoxyribonuclease specific for an altered base (EC 3.1.25); an endoribonuclease producing a 5′-phosphomonoester (EC 3.1.26); an endoribonuclease producing a 3′-phosphomonoester (EC 3.1.27); an endoribonuclease active with a ribonucleic acid and/or a deoxyribonucleic acid and producing a 5′-phosphomonoester (EC 3.1.30); an endoribonuclease active with a ribonucleic acid and/or a deoxyribonucleic acid and producing a 3′-phosphomonoester (EC 3.1.31); or a combination thereof.

Examples of a carboxylic ester hydrolase (EC 3.1.1) include a carboxylesterase (EC 3.1.1.1); an arylesterase (EC 3.1.1.2); a triacylglycerolipase (EC 3.1.1.3); a phospholipase A2 (EC 3.1.1.4); a lysophospholipase (EC 3.1.1.5); an acetylesterase (EC 3.1.1.6); an acetylcholinesterase (EC 3.1.1.7); a cholinesterase (EC 3.1.1.8); a tropinesterase (EC 3.1.1.10); a pectinesterase (EC 3.1.1.11); a sterol esterase (EC 3.1.1.13); a chlorophyllase (EC 3.1.1.14); a L-arabinonolactonase (EC 3.1.1.15); a gluconolactonase (EC 3.1.1.17); an uronolactonase (EC 3.1.1.19); a tannase (EC 3.1.1.20); a retinyl-palmitate esterase (EC 3.1.1.21); a hydroxybutyrate-dimer hydrolase (EC 3.1.1.22); an acylglycerol lipase (EC 3.1.1.23); a 3-oxoadipate enol-lactonase (EC 3.1.1.24); a 1,4-lactonase (EC 3.1.1.25); a galactolipase (EC 3.1.1.26); a 4-pyridoxolactonase (EC 3.1.1.27); an acylcarnitine hydrolase (EC 3.1.1.28); an aminoacyl-tRNA hydrolase (EC 3.1.1.29); a D-arabinonolactonase (EC 3.1.1.30); a 6-phosphogluconolactonase (EC 3.1.1.31); a phospholipase A1 (EC 3.1.1.32); a 6-acetylglucose deacetylase (EC 3.1.1.33); a lipoprotein lipase (EC 3.1.1.34); a dihydrocoumarin hydrolase (EC 3.1.1.35); a limonin-D-ring-lactonase (EC 3.1.1.36); a steroid-lactonase (EC 3.1.1.37); a triacetate-lactonase (EC 3.1.1.38); an actinomycin lactonase (EC 3.1.1.39); an orsellinate-depside hydrolase (EC 3.1.1.40); a cephalosporin-C deacetylase (EC 3.1.1.41); a chlorogenate hydrolase (EC 3.1.1.42); a α-amino-acid esterase (EC 3.1.1.43); a 4-methyloxaloacetate esterase (EC 3.1.1.44); a carboxymethylenebutenolidase (EC 3.1.1.45); a deoxylimonate A-ring-lactonase (EC 3.1.1.46); a 1-alkyl-2-acetylglycerophosphocholine esterase (EC 3.1.1.47); a fusarinine-C ornithinesterase (EC 3.1.1.48); a sinapine esterase (EC 3.1.1.49); a wax-ester hydrolase (EC 3.1.1.50); a phorbol-diester hydrolase (EC 3.1.1.51); a phosphatidylinositol deacylase (EC 3.1.1.52); a sialate O-acetylesterase (EC 3.1.1.53); an acetoxybutynylbithiophene deacetylase (EC 3.1.1.54); an acetylsalicylate deacetylase (EC 3.1.1.55); a methylumbelliferyl-acetate deacetylase (EC 3.1.1.56); a 2-pyrone-4,6-dicarboxylate lactonase (EC 3.1.1.57); a N-acetylgalactosaminoglycan deacetylase (EC 3.1.1.58); a juvenile-hormone esterase (EC 3.1.1.59); a bis(2-ethylhexyl)phthalate esterase (EC 3.1.1.60); a protein-glutamate methylesterase (EC 3.1.1.61); a 11-cis-retinyl-palmitate hydrolase (EC 3.1.1.63); an all-trans-retinyl-palmitate hydrolase (EC 3.1.1.64); a L-rhamnono-1,4-lactonase (EC 3.1.1.65); a 5-(3,4-diacetoxybut-1-ynyl)-2,2′-bithiophene deacetylase (EC 3.1.1.66); a fatty-acyl-ethyl-ester synthase (EC 3.1.1.67); a xylono-1,4-lactonase (EC 3.1.1.68); a cetraxate benzylesterase (EC 3.1.1.70); an acetylalkylglycerol acetylhydrolase (EC 3.1.1.71); an acetylxylan esterase (EC 3.1.1.72); a feruloyl esterase (EC 3.1.1.73); a cutinase (EC 3.1.1.74); a poly(3-hydroxybutyrate) depolymerase (EC 3.1.1.75); a poly(3-hydroxyoctanoate) depolymerase (EC 3.1.1.76); an acyloxyacyl hydrolase (EC 3.1.1.77); a polyneuridine-aldehyde esterase (EC 3.1.1.78); a hormone-sensitive lipase (EC 3.1.1.79); an acetylajmaline esterase (EC 3.1.1.80); a quorum-quenching N-acyl-homoserine lactonase (EC 3.1.1.81); a pheophorbidase (EC 3.1.1.82); a monoterpene ∈-lactone hydrolase (EC 3.1.1.83); or a combination thereof.

Examples of an enzyme that acts on a carbon-nitrogen bond, other than a peptide bond (EC 3.5) include an enzyme acting on a linear amide (EC 3.5.1); a cyclic amide (EC 3.5.2); a linear amidine (EC 3.5.3); a cyclic amidine (EC 3.5.4); a nitrile (EC 3.5.5); an other compound (EC 3.5.99); or a combination thereof. Examples of an enzyme that catalyzes a reaction on a carbon-nitrogen bond of a non-peptide linear amide (EC 3.5.1) include an asparaginase (EC 3.5.1.1); a glutaminase (EC 3.5.1.2); a ω-amidase (EC 3.5.1.3); an amidase (EC 3.5.1.4); a urease (EC 3.5.1.5); a β-ureidopropionase (EC 3.5.1.6); a ureidosuccinase (EC 3.5.1.7); a formylaspartate deformylase (EC 3.5.1.8); an arylformamidase (EC 3.5.1.9); a formyltetrahydrofolate deformylase (EC 3.5.1.10); a penicillin amidase (EC 3.5.1.11); a biotimidase (EC 3.5.1.12); an aryl-acylamidase (EC 3.5.1.13); an aminoacylase (EC 3.5.1.14); an aspartoacylase (EC 3.5.1.15); an acetylornithine deacetylase (EC 3.5.1.16); an acyl-lysine deacylase (EC 3.5.1.17); a succinyl-diaminopimelate desuccinylase (EC 3.5.1.18); a nicotinamidase (EC 3.5.1.19); a citrullinase (EC 3.5.1.20); a N-acetyl-β-alanine deacetylase (EC 3.5.1.21); a pantothenase (EC 3.5.1.22); a ceramidase (EC 3.5.1.23); a choloylglycine hydrolase (EC 3.5.1.24); a N-acetylglucosamine-6-phosphate deacetylase (EC 3.5.1.25); a N4-(β-N-acetylglucosaminyl)-L-asparaginase (EC 3.5.1.26); a N-formylmethionylaminoacyl-tRNA deformylase (EC 3.5.1.27); a N-acetylmuramoyl-L-alanine amidase (EC 3.5.1.28); a 2-(acetamidomethylene)succinate hydrolase (EC 3.5.1.29); a 5-aminopentanamidase (EC 3.5.1.30); a formylmethionine deformylase (EC 3.5.1.31); a hippurate hydrolase (EC 3.5.1.32); a N-acetylglucosamine deacetylase (EC 3.5.1.33); a D-glutaminase (EC 3.5.1.35); a N-methyl-2-oxoglutaramate hydrolase (EC 3.5.1.36); a glutamin-(asparagin-)ase (EC 3.5.1.38); an alkylamidase (EC 3.5.1.39); an acylagmatine amidase (EC 3.5.1.40); a chitin deacetylase (EC 3.5.1.41); a nicotinamide-nucleotide amidase (EC 3.5.1.42); a peptidyl-glutaminase (EC 3.5.1.43); a protein-glutamine glutaminase (EC 3.5.1.44); a 6-aminohexanoate-dimer hydrolase (EC 3.5.1.46); a N-acetyldiaminopimelate deacetylase (EC 3.5.1.47); an acetylspermidine deacetylase (EC 3.5.1.48); a formamidase (EC 3.5.1.49); a pentanamidase (EC 3.5.1.50); a 4-acetamidobutyryl-CoA deacetylase (EC 3.5.1.51); a peptide-N4-(N-acetyl-β-glucosaminy)asparagines amidase (EC 3.5.1.52); a N-carbamoylputrescine amidase (EC 3.5.1.53); an allophanate hydrolase (EC 3.5.1.54); a long-chain-fatty-acyl-glutamate deacylase (EC 3.5.1.55); a N,N-dimethylformamidase (EC 3.5.1.56); a tryptophanamidase (EC 3.5.1.57); a N-benzyloxycarbonylglycine hydrolase (EC 3.5.1.58); a N-carbamoylsarcosine amidase (EC 3.5.1.59); a N-(long-chain-acyl)ethanolamine deacylase (EC 3.5.1.60); a mimosinase (EC 3.5.1.61); an acetylputrescine deacetylase (EC 3.5.1.62); a 4-acetamidobutyrate deacetylase (EC 3.5.1.63); a Na-benzyloxycarbonylleucine hydrolase (EC 3.5.1.64); a theanine hydrolase (EC 3.5.1.65); a 2-(hydroxymethyl)-3-(acetamidomethylene)succinate hydrolase (EC 3.5.1.66); a 4-methyleneglutaminase (EC 3.5.1.67); a N-formylglutamate deformylase (EC 3.5.1.68); a glycosphingolipid deacylase (EC 3.5.1.69); an aculeacin-A deacylase (EC 3.5.1.70); a N-feruloylglycine deacylase (EC 3.5.1.71); a D-benzoylarginine-4-nitroanilide amidase (EC 3.5.1.72); a carnitinamidase (EC 3.5.1.73); a chenodeoxycholoyltaurine hydrolase (EC 3.5.1.74); a urethanase (EC 3.5.1.75); an arylalkyl acylamidase (EC 3.5.1.76); a N-carbamoyl-D-amino acid hydrolase (EC 3.5.1.77); a glutathionylspermidine amidase (EC 3.5.1.78); a phthalyl amidase (EC 3.5.1.79); a N-acetylgalactosamine-6-phosphate deacetylase (EC 3.5.1.80); a N-acyl-D-amino-acid deacylase (EC 3.5.1.81); a N-acyl-D-glutamate deacylase (EC 3.5.1.82); a N-acyl-D-aspartate deacylase (EC 3.5.1.83); a biuret amidohydrolase (EC 3.5.1.84); a (S)—N-acetyl-1-phenylethylamine hydrolase (EC 3.5.1.85); a mandelamide amidase (EC 3.5.1.86); a N-carbamoyl-L-amino-acid hydrolase (EC 3.5.1.87); a peptide deformylase (EC 3.5.1.88); a N-acetylglucosaminylphosphatidylinositol deacetylase (EC 3.5.1.89); an adenosylcobinamide hydrolase (EC 3.5.1.90); a N-substituted formamide deformylase (EC 3.5.1.91); a pantetheine hydrolase (EC 3.5.1.92); a glutaryl-7-aminocephalosporanic-acid acylase (EC 3.5.1.93); a γ-glutamyl-γ-aminobutyrate hydrolase (EC 3.5.1.94); a N-malonylurea hydrolase (EC 3.5.1.95); a succinylglutamate desuccinylase (EC 3.5.1.96); an acyl-homoserine-lactone acylase (EC 3.5.1.97); a histone deacetylase (EC 3.5.1.98); or a combination thereof. Examples of an enzyme that catalyzes a reaction on a carbon-nitrogen bond of a non-peptide cyclic amide (EC 3.5.2) include a barbiturase (EC 3.5.2.1); a dihydropyrimidinase (EC 3.5.2.2); a dihydroorotase (EC 3.5.2.3); a carboxymethylhydantoinase (EC 3.5.2.4); an allantoinase (EC 3.5.2.5); a β-lactamase (EC 3.5.2.6); an imidazolonepropionase (EC 3.5.2.7); a 5-oxoprolinase (ATP-hydrolysing) (EC 3.5.2.9); a creatininase (EC 3.5.2.10); a L-lysine-lactamase (EC 3.5.2.11); a 6-aminohexanoate-cyclic-dimer hydrolase (EC 3.5.2.12); a 2,5-dioxopiperazine hydrolase (EC 3.5.2.13); a N-methylhydantoinase (ATP-hydrolysing) (EC 3.5.2.14); a cyanuric acid amidohydrolase (EC 3.5.2.15); a maleimide hydrolase (EC 3.5.2.16); a hydroxyisourate hydrolase (EC 3.5.2.17); an enamidase (EC 3.5.2.18); or a combination thereof.

Examples of an enzyme that acts on an acid anhydride (EC 3.6) include an enzyme acting on: a phosphorus-containing anhydride (EC 3.6.1); a sulfonyl-containing anhydride (EC 3.6.2); an acid anhydride catalyzing transmembrane movement of a substance (EC 3.6.3); an acid anhydride involved in cellular and/or subcellular movement (EC 3.6.4); a GTP involved in cellular and/or subcellular movement (EC 3.6.5); or a combination thereof.

A lyase catalyzes the cleavage of a chemical bond by reactions other than hydrolysis and/or oxidation. A lyase may be classified based on the chemical bond cleaved. Examples of a lyase include a lyase that catalyzes the cleavage of a carbon-carbon bond, (EC 4.1); a carbon-oxygen bond, (EC 4.2); a carbon-nitrogen bond, (EC 4.3); a carbon-sulfur bond, (EC 4.4); a carbon-halide bond, (EC 4.5); a phosphorus-oxygen bond, (EC 4.6); an other lyase, (EC 4.99); or a combination thereof.

An isomerase catalyzes a change within one molecule. Examples of an isomerase include a racemase and/or an epimerase, (EC 5.1); a cis-trans-isomerase, (EC 5.2); an intramolecular isomerase, (EC 5.3); an intramolecular transferase, (EC 5.4); an intramolecular lyase, (EC 5.5); an other isomerases, (EC 5.99); or a combination thereof.

A ligase catalyzes the formation of a chemical bond between two substrates with the hydrolysis of a diphosphate bond of a triphosphate such as ATP. A ligase may be classified based on the chemical bond created. Examples of a lyase include a ligase that form a carbon-oxygen bond, (EC 6.1); a carbon-sulfur bond, (EC 6.2); a carbon-nitrogen bond, (EC 6.3); a carbon-carbon bond, (EC 6.4); a phosphoric ester bond, (EC 6.5); or a combination thereof.

1. Lipolytic Enzymes

An enzyme in various embodiments comprises a lipolytic enzyme, which as used herein comprises an enzyme that catalyzes a reaction or series of reactions on a lipid substrate. In many embodiments, a lipolytic enzyme produces one or more products that are more soluble in a liquid component such as a polar liquid component (e.g., water); absorb easier into a material formulation than the lipid substrate. In some embodiments, the enzyme catalyzes hydrolysis of a fatty acid bond (e.g., an ester bond). In other embodiments, the products produced comprise a carboxylic acid moiety (e.g., a free fatty acid), an alcohol moiety (e.g., a glycerol), or a combination thereof. In specific embodiments, at least one product may be relatively more soluble in an aqueous media (e.g., a water comprising detergent) than the substrate.

As used herein, a “lipid” comprises a hydrophobic and/or an amphipathic organic molecule extractable with a non-aqueous solvent. Examples of a lipid include a triglyceride; a diglyceride; a monoglyceride; a phospholipid; a glycolipid (e.g., galactolipid); a steroid (e.g., cholesterol); a wax; a fat-soluble vitamin (e.g., vitamin A, D, E, K); a petroleum based material, such as, for example, a hydrocarbon composition such as gasoline, a crude petroleum oil, a petroleum grease, etc.; or a combination thereof. A lipid may comprise a combination (mixture) of lipids, such as a grease comprising both a fatty acid based lipid and a petroleum based lipid. A lipid may comprise an a polar (“nonpolar”) lipid (e.g., a hydrocarbons, a carotene), a polar lipid (e.g., triacylglycerol, a retinol, a wax, a sterol), or a combination thereof. In some embodiments, a polar lipid may possess partial solubility in water (e.g., a lysophospholipid). Because of the prevalence of these types of lipids in activities such as, for example, a restaurant food preparation and a counterpart use in a household application, a material formulation may be formulated to comprise one or more lipolytic enzymes to promote lipid removal from a material formulation contaminated with a lipid in these and/or other environments.

Lipolytic enzymes have been identified in cells across the phylogenetic categories, and purified for analysis and/or use in commercial applications (Brockerhoff, Hans and Jensen, Robert G. “Lipolytic Enzymes,” 1974). Further, numerous nucleotide sequences for lipolytic enzymes have been isolated, the encoded protein sequence determined, and in many cases the nucleotide sequences recombinantly expressed for high level production of a lipolytic enzyme (e.g., a lipase), particularly for isolation, purification and subsequent use in an industrial/commercial application [“Lipases their Structure, Biochemistry and Application” (Paul Woolley and Steffen B. Peterson, Eds.) 1994].

Many lipolytic enzymes are classified as an alpha/beta fold hydrolase (“alpha/beta hydrolase”), due to a structural configuration generally comprising an 8 member beta pleated sheet, where many sheets are parallel, with several alpha helices on both sides of the sheet. A lipolytic enzyme's amino acid sequence commonly comprises Ser, Glu/Asp, His active site residues (e.g., Ser152, Asp176, and His263 by human pancreatic numbering). The Ser may be comprised in a GXSXG substrate binding consensus sequence for many types of lipolytic enzymes, with a GGYSQGXA sequence being present in a cutinase. The active site serine may be at a turn between a beta-strand and an alpha helix, and these lipolytic enzymes are classified as serine esterases. A substitution at the 1^{st} position Gly (e.g., Thr) has been identified in some lipolytic enzymes. Often a Pro residue may be found at the residues 1 and 4 down from the Asp, and the His may be typically within a CXHXR sequence. A lipolytic enzyme generally comprises an alpha helix flap (a.k.a. “lid”) region (around amino acid residues 240-260 by human pancreatic lipase numbering) covering the active site, with a conserved tryptophan in this region in proximity of the active site serine in many lipolytic enzymes [In “Advances in Protein Chemistry, Volume 45 Lipoproteins, Apolipoproteins, and Lipases.” (Anfinsen, C. B., Edsall, J. T., Richards, Frederic, R. M., Eisenberg, D. S., and Schumaker, V. N. Eds.) Academic Press, Inc., San Diego, Calif., pp. 1-152, 1994; “Lipases their Structure, Biochemistry and Application” (Paul Woolley and Steffen B. Peterson, Eds.), pp. 1-243-270, 337-354, 1994.]. Any such alpha/beta hydrolase, particularly one possessing a lipolytic activity, may be used.

A lipolytic alpha/beta hydrolase's catalysis usually depends upon and/or becomes stimulated by interfacial activation, which refers to the contact of such an enzyme with an interface where two layers of materials with differing hydrophobic/hydrophilic character meet, such as a water/oil interface of a micelle and/or an emulsion, an air/water interface, and/or a solid carrier/organic solvent interface of an immobilized enzyme. Interfacial activation may result from lipid substrate forming an ordered confirmation in a localized hydrophobic environment, so that the substrate more easily binds a lipolytic enzyme than a lipid substrate's conformation in a hydrophilic environment. A conformational change in the flap region due to contact with the interface allows substrate binding in many alpha/beta hydrolases. Cutinase comprises a lipolytic alpha/beta hydrolase that may be not substantially enhanced by interfacial activation. A cutinase generally lacks a lid, and may possess the ability to bury an aliphatic fatty acid chain in the active site cleft without the charge effects of an interface prompting a conformational change in the enzyme [In “Engineering of/with Lipases” (F. Xavier Malcata., Ed.), pp. 125-142, 1996].

In general embodiments, a lipolytic enzyme contemplated for use hydrolyzes an ester of a glycerol based lipid (e.g., a triglyceride, a phospholipid). Glycerol typically comprises a naturally produced alcohol having a 3 carbon backbone with 3 alcohol moieties (positions 1, 2, and 3). One or more of these positions are often esterified with a fatty acid in many naturally produced and/or synthetic lipids. Common examples of a triglyceride include a fat, which comprises a solid at room temperature; or an oil, which comprises a liquid at room temperature. As used herein, a “fatty acid” (“FA”) refers to saturated, monounsaturated, or polyunsaturated aliphatic acid. A short chain fatty acid comprises about 2 to about 6 carbons (“C2 to C6”) in the carboxyl moiety and the main aliphatic carbon chain, a medium chain fatty acid comprises about 8 to about 10 carbons in the acid and main chain; and a long chain fatty acid comprises about 12 or more carbons (e.g., 12 to about 60 carbons). Of course, various derivative equivalents are contemplated, with one or more main chain carbons substituted by another element (e.g., oxygen). A short chain fatty acid generally possesses solubility in water and other polar solvents, but solubility tends to decrease with increased carbon chain length in polar solvents, though solubility in non-polar solvents tends to increase. A common solvent for a medium and/or a long chain fatty acid includes an acetone, an acetic acid, an acetonitrile, a benzene, a chloroform, a chyclohexane, an alcohol (e.g., ethanol, methanol), or a combination thereof. A lipolytic enzyme hydrolyzes an ester at one or more of glycerol's alcohol position(s) (e.g., a 1,3 lipase), though a lipolytic enzyme often hydrolyzes a non-glycerol ester of an alcohol other than glycerol. For example, a naturally produced wax comprises a fatty acid ester of ethylene glycol, which has a 2 carbon backbone and 2 alcohol moieties, where one or both of the alcohol moiety(s) are esterified with a fatty acid.

In other lipids, a fatty acid forms an ester with an alcohol group of a non-glycerol and/or an ethylene glycol molecule, such as sterol lipid (e.g., cholesterol), and an enzyme that catalyzes the formation and/or cleavage of that linkage may be considered to comprise a lipolytic enzyme (e.g., a sterol hydrolase). Conversely, in some cases, one or more hydroxyl moiety(s) of an alcohol (e.g., a glycerol, an ethylene glycol, etc.) comprise a fatty acid and one or more hydroxyl moiety(s) comprise an ester of a chemical structure other than a fatty acid, and an enzyme that catalyzes hydrolysis and/or cleavage of the non-FA linkage comprises a lipolytic enzyme (e.g., a phospholipase). For example, a phospholipid (“phosphoglyceride”) comprises a diglyceride with the 3^{rd} remaining position esterified to a phosphate group. The phosphate moiety may be esterified to a hydrophilic moiety such as a polyhydroxyl alcohol (e.g., a glycerol, an inositol) and/or an amino alcohol (e.g., a choline, a serine, an ethanolamine). Examples of a phospholipid includes a phosphatidic acid (“PA”), a phosphatidylcholine (“PC,” “lecithin”), a phosphotidyl ethanolamine (“PE,” “cephalin”), a phosphotidylglycerol (“PG”), a phosphotidylinositol (“PI,” “monophosphoinositide”), a phosphotidylserine (“PE,” “serine”), a phosphotidylinositol 4,5-diphosphate (“PIP₂,” “triphosphoinositide”), a diphosphotidylglycerol (“DPG,” “cardiolipin”), or a combination thereof. In some cases, an alcohol (e.g., a glycerol, an ethylene glycol) comprises a non-ester linkage to a fatty acid, and a lipolytic enzyme may act on that substrate to hydrolyze that linkage. For example, sphingomyelin comprises a glycerol having a fatty acid amide bond and 2 phosphate ester bonds, and a lipolytic enzyme may cleave the amide linkage.

An enzyme may be identified and referred to by the primary catalytic function (E.C. classification), but often catalyze another reaction, and examples of such an enzyme may be referred to herein (e.g., a carboxylesterase/lipase) based on the multiple activities. Mixtures of enzymes (e.g., lipolytic enzymes) may be used to broaden the range of effective activity against various substrates, effectiveness in differing material compositions, and/or environmental conditions. For example, in some embodiments, a material formulation comprising one or more enzymes lipolytic enzyme(s) may possess the ability to cleave (e.g., hydrolyze) all positions of an alcohol for ease of removal of the product(s) of the reaction. In some embodiments, a multifunction enzyme may be used instead a plurality of enzymes to expand the range of different substrates that are acted upon, though a plurality of single and/or multifunctional enzymes may be used as well. In another example, a plurality of different lipolytic enzymes and organophosphorus compound degrading enzymes derived from a mesophile and an extremophile may be incorporated into a material formulation to expand the catalytic effectiveness against various substrates in differing temperature conditions experienced in an outdoor application and/or near a heat source.

Though a lipolytic enzyme often produces a product that may be more aqueous soluble and/or removable after a single chemical reaction, in some aspects, a series of enzyme reactions releases a fatty acid and/or degrades a lipid, such as in the case of a combination of a sphingomyelin phosphodiesterase that produces a N-acylsphingosine from a sphingomyelin phospholipid, followed by a ceramidase hydrolyzing an amide bond in a N-acylsphingosine to produce a free fatty acid and a sphingosine.

Often an enzyme such as a lipolytic enzyme prefers an isomer and/or enantiomer of a particular lipid (e.g., a triglyceride comprising one sequence of different fatty acids esters out of many that are possible), but in some embodiments a material formulation comprising one or more lipolytic enzymes may possess the ability to hydrolyze a plurality of lipid isomers and/or enantiomers for a broader range of substrates than a single enzyme.

In general embodiments, a lipolytic enzyme comprises a hydrolase. A hydrolase generally comprises an esterase, a ceramidase (EC 3.5.1.23), or a combination thereof. Examples of an esterase comprise those identified by enzyme commission number (EC 3.1): a carboxylic ester hydrolase, (EC 3.1.3), a phosphoric monoester hydrolase (EC 3.1.3), a phosphoric diester hydrolase (EC 3.1.4), or a combination thereof. A carboxylic ester hydrolase catalyzes the hydrolytic cleavage of an ester to produce an alcohol and a carboxylic acid product. A phosphoric monoester hydrolase catalyzes the hydrolytic cleavage of an O—P ester bond. A “phosphoric diester hydrolase” catalyzes the hydrolytic cleavage of a phosphate group's phosphorus atom and two other moieties over two ester bonds. A “ceramidase” hydrolyzes the N-acyl bond of ceramide to release a fatty acid and sphingosine. Examples of a lipolytic esterase and a ceramidase include a carboxylesterase (EC 3.1.1.1), a lipase (EC 3.1.1.3), a lipoprotein lipase (EC 3.1.1.34), an acylglycerol lipase (EC 3.1.1.23), a hormone-sensitive lipase (EC 3.1.1.79), a phospholipase A₁ (EC 3.1.1.32), a phospholipase A₂ (EC 3.1.1.4), a phosphatidylinositol deacylase (EC 3.1.1.52), a phospholipase C (EC 3.1.4.3), a phospholipase D (EC 3.1.4.4), a phosphoinositide phospholipase C (EC 3.1.4.11), a phosphatidate phosphatase (EC 3.1.3.4), a lysophospholipase (EC 3.1.1.5), a sterol esterase (EC 3.1.1.13), a galactolipase (EC 3.1.1.26), a sphingomyelin phosphodiesterase (EC 3.1.4.12), a sphingomyelin phosphodiesterase D (EC 3.1.4.41), a ceramidase (EC 3.5.1.23), a wax-ester hydrolase (EC 3.1.1.50), a fatty-acyl-ethyl-ester synthase (EC 3.1.1.67), a retinyl-palmitate esterase (EC 3.1.1.21), a 11-cis-retinyl-palmitate hydrolase (EC 3.1.1.63), an all-trans-retinyl-palmitate hydrolase (EC 3.1.1.64), a cutinase (EC 3.1.1.74), an acyloxyacyl hydrolase (EC 3.1.1.77), a petroleum lipolytic enzyme, or a combination thereof.

a. Carboxylesterases

Carboxylesterase (EC 3.1.1.1) has been also referred to in that art as “carboxylic-ester hydrolase,” “ali-esterase,” “B-esterase,” “monobutyrase,” “cocaine esterase,” “procaine esterase,” “methylbutyrase,” “vitamin A esterase,” “butyryl esterase,” “carboxyesterase,” “carboxylate esterase,” “carboxylic esterase,” “methylbutyrate esterase,” “triacetin esterase,” “carboxyl ester hydrolase,” “butyrate esterase,” “methylbutyrase,” “α-carboxylesterase,” “propionyl esterase,” “nonspecific carboxylesterase,” “esterase D,” “esterase B,” “esterase A,” “serine esterase,” “carboxylic acid esterase,” and/or “cocaine esterase.” Carboxylesterase catalyzes the reaction: carboxylic ester+H₂O=an alcohol+a carboxylate. In many embodiments, the carboxylate comprises a fatty acid. In additional aspects, the fatty acid comprises about 10 or less carbons, to differentiate its preferred substrate and classification from a lipase, though a carboxylesterase (e.g., a microsome carboxylesterase) may possess the catalytic activity of an arylesterase, a lysophospholipase, an acetylesterase, an acylglycerol lipase, an acylcarnitine hydrolase, a palmitoyl-CoA hydrolase, an amidase, an aryl-acylamidase, a vitamin A esterase, or a combination thereof. Carboxylesterase producing cells and methods for isolating a carboxylesterase from a cellular material and/or a biological source have been described [see, for example, Augusteyn, R. C. et al., 1969; Horgan, D. J., et al., 1969; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for a wild-type carboxylesterase and/or a functional equivalent amino acid sequence for producing a carboxylesterase and/or a functional equivalent include Protein database bank entries: 1AUO, 1AUR, 1CI8, 1CI9, 1EVQ, 1JJI, 1K4Y, 1L7Q, 1L7R, 1MX1, 1MX5, 1MX9, 1QZ3, 1R1D, 1TQH, 1U4N, 1YA4, 1YA8, 1YAH, 1YAJ, 2C7B, 2DQY, 2DQZ, 2DR0, 2FJ0, 2H1I, 2H7C, 2HM7, 2HRQ, 2HRR, 2JEY, 2JEZ, 2JF0, 2O7R, 2O7V, 2OGS, 2OGT, and/or 2R11.

b. Lipases

Lipase (EC 3.1.1.3) has been also referred to in that art as “triacylglycerol acylhydrolase,” “triacylglycerol lipase,” “triglyceride lipase,” “tributyrase,” “butyrinase,” “glycerol ester hydrolase,” “tributyrinase,” “Tween hydrolase,” “steapsin,” “triacetinase,” “tributyrin esterase,” “Tweenase,” “amno N-AP,” “Takedo 1969-4-9,” “Meito MY 30,” “Tweenesterase,” “GA 56,” “capalase L,” “triglyceride hydrolase,” “triolein hydrolase,” “tween-hydrolyzing esterase,” “amano CE,” “cacordase,” “triglyceridase,” “triacylglycerol ester hydrolase,” “amano P,” “amano AP,” “PPL,” “glycerol-ester hydrolase,” “GEH,” “meito Sangyo OF lipase,” “hepatic lipase,” “lipazin,” “post-heparin plasma protamine-resistant lipase,” “salt-resistant post-heparin lipase,” “heparin releasable hepatic lipase,” “amano CES,” “amano B,” “tributyrase,” “triglyceride lipase,” “liver lipase,” and/or “hepatic monoacylglycerol acyltransferase.” A lipase catalyzes the reaction: triacylglycerol+H₂O=diacylglycerol+a carboxylate. In many embodiments, the carboxylate comprises a fatty acid. Lipase and/or co-lipase producing cells and methods for isolating a lipase and/or a co-lipase from a cellular material and/or a biological source have been described, [see, for example, Korn, E. D. and Quigley., 1957; Lynn, W. S, and Perryman, N. C. 1960; Tani, T. and Tominaga, Y. J., 1991; Sugihara, A. et al., 1992; in “Methods and Molecular Biology, Volume 109 Lipase and Phospholipase Protocols.” (Mark Doolittle and Karen Reue, Eds.), pp. 157-164, 1999; pancreatic lipase via recombinant expression in a baculoviral system in “Methods and Molecular Biology, Volume 109 Lipase and Phospholipase Protocols.” (Mark Doolittle and Karen Reue, Eds.), pp. 187-213, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 49-262, 307-328, 365-416, 1984; In “Lipases and Phospholipases in Drug Development from Biochemistry to Molecular Pharmacology.” (Müller, G. and Petry, S. Eds.) pp. 1-22, 2004], and may be used in conjunction with the disclosures herein.

A lipase may often catalyze the hydrolysis of short and/or medium chain fatty acid(s) less than about 12 carbons (“12C”), but has a preference and/or specificity for about 12C or greater fatty acid(s). In contrast, a lipolytic enzyme classified as a carboxylesterase prefers short and/or medium chain fatty acid(s), though some carboxylesterases may also hydrolyze esters of longer fatty acids. The chain length preference for a lipase may be applicable to the other lipolytic fatty acid esterase(s) and/or a ceramidase, other than a carboxylesterase unless otherwise noted.

A lipase may be obtained from a commercial vendor, such as a type VII lipase from Candida rugosa (Sigma-Aldrich product no. L1754; ≧700 unit/mg solid; CAS No. 9001-62-1) comprising lactose; a Lipoase (Novozymes; Lipolase 100 L, Type EX), which typically comprises about 2% (w/w) lipase from Thermomyces lanuginosus (CAS No. 9001-62-1), about 25% propylene glycol (CAS No. 57-55-6), about 73% water, and about 0.5% calcium chloride. An enzyme stabilizing compound such as a propylene glycol and/or a sucrose may promote a property such as enzyme activity/stability in a material formulation (e.g., a water-borne paint, a 2 k epoxy system).

A mammalian lipase may be classified into one of four groups: gastric, hepatic, lingual, and pancreatic, and has homology to lipoprotein lipase. A pancreatic lipase generally are inactivated by a bile salt, which comprise an amphiphilic molecule found in an animal intestine that may bind a lipid and confer a negative charge that inhibits a pancreatic lipase. A colipase comprises a protein that binds a pancreatic lipase and reactivates it in the presence of a bile salt [In “Engineering of/with Lipases” (F. Xavier Malcata., Ed.) p. 168, 1996]. In some embodiments, a co-lipase may be combined with a pancreatic lipase in a composition to promote a lipase's (e.g., a pancreatic lipase) activity.

Structural information for a wild-type lipase and/or a functional equivalent amino acid sequence for producing a lipase and/or a functional equivalent include Protein database bank entries: 1AKN, 1BU8, 1CRL, 1CUA, 1CUB, 1CUC, 1CUD, 1CUE, 1CUF, 1CUG, 1CUH, 1CUI, 1CUJ, 1CUU, 1CUV, 1CUW, 1CUX, 1CUY, 1CUZ, 1CVL, 1DT3, 1DT5, 1DTE, 1DU4, 1EIN, 1ETH, 1EX9, 1F6W, 1FFA, 1FFB, 1FFC, 1FFD, 1FFE, 1GPL, 1GT6, 1GZ7, 1HLG, 1HPL, 1HQD, 1I6W, 1ISP, 1JI3, 1JMY, 1K 8Q, 1KU0, 1LBS, 1LBT, 1LGY, 1LLF, 1LPA, 1LPB, 1LPM, 1LPN, 1LPO, 1LPP, 1LPS, 1N8S, 1OIL, 1QGE, 1R4Z, 1R50, 1RP1, 1T2N, 1T4M, 1TAH, 1TCA, 1TCB, 1TCC, 1TGL, 1THG, 1TIA, 1TIB, 1TIC, 1TRH, 1YS1, 1YS2, 2DSN, 2ES4, 2FX5, 2HIH, 2LIP, 2NW6, 2ORY, 2OXE, 2PPL, 2PVS, 2QUA, 2QUB, 2QXT, 2QXU, 2VEO, 2Z5G, 2Z8X, 2Z8Z, 3D2A, 3D2B, 3D2C, 3LIP, 3TGL, 4LIP, 4TGL, 5LIP, and/or 5TGL.

c. Lipoprotein Lipases

Lipoprotein lipase (EC 3.1.1.34) has been also referred to in that art as “triacylglycero-protein acylhydrolase,” “clearing factor lipase,” “diglyceride lipase,” “diacylglycerol lipase,” “postheparin esterase,” “diglyceride lipase,” “postheparin lipase,” “diacylglycerol hydrolase,” and/or “lipemia-clearing factor.” A lipoprotein lipase's biological function comprises hydrolyzing a triglyceride found in an animal lipoprotein. Lipoprotein lipase catalyzes the reaction: triacylglycerol+H₂O=diacylglycerol+a carboxylate. This enzyme also acts on diacylglycerol to produce a monoacylglycerol. An apolipoprotein activates lipoprotein lipase [“Lipases” (Borgstrom, B. and Brockman, H. L., Eds), p. 228-230, 1984]. In some embodiments, a protein such as apolipoprotein may be combined with a lipoprotein lipase. Lipoprotein lipase producing cells and methods for isolating a lipoprotein lipase from a cellular material and/or a biological source have been described, [see, for example, Egelrud, T. and Olivecrona, T., 1973; Greten, H. et al., 1970; in “Methods and Molecular Biology, Volume 109 Lipase and Phospholipase Protocols.” (Mark Doolittle and Karen Reue, Eds.), pp. 133-143, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 263-306, 1984], and may be used in conjunction with the disclosures herein.

d. Acylglycerol Lipases

Acylglycerol lipase (EC 3.1.1.23) has been also referred to in that art as “glycerol-ester acylhydrolase,” “monoacylglycerol lipase,” “monoacylglycerolipase,” “monoglyceride lipase,” “monoglyceride hydrolase,” “fatty acyl monoester lipase,” “monoacylglycerol hydrolase,” “monoglyceridyl lipase,” and/or “monoglyceridase.” Acylglycerol lipase catalyzes a glycerol monoester's hydrolysis, particularly a fatty acid ester's hydrolysis. Acylglycerol lipase producing cells and methods for isolating an acylglycerol lipase from a cellular material and/or a biological source have been described, [see, for example, Mentlein, R. et al., 1980; Pope, J. L. et al., 1966; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

e. Hormone-Sensitive Lipases

Hormone-sensitive lipase (EC 3.1.1.79) has been also referred to in that art as “diacylglycerol acylhydrolase” and/or “HSL.” Hormone-sensitive lipase catalyzes the reactions, in order of catalytic preference: diacylglycerol+H₂O=monoacylglycerol+a carboxylate; triacylglycerol+H₂O=diacylglycerol+a carboxylate; and monoacylglycerol+H₂O=glycerol+a carboxylate. A hormone-sensitive lipase generally may be also active against a steroid fatty acid ester and/or a retinyl ester, and/or has a preference for a 1- or a 3-ester bond of an acylglycerol substrate. Hormone-sensitive lipase producing cells and methods for isolating a hormone-sensitive lipase from a cellular material and/or a biological source have been described, [see, for example, Tsujita, T. et al., 1989; Fredrikson, G., et al., 1981; via recombinant expression in a baculoviral system in “Methods and Molecular Biology, Volume 109 Lipase and Phospholipase Protocols.” (Mark Doolittle and Karen Reue, Eds.), pp. 165-175, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

f. Phospholipases A₁

Phospholipase A₁ (EC 3.1.1.32) has been also referred to in that art as “phosphatidylcholine 1-acylhydrolase.” A phospholipase A₁ catalyzes the reaction: phosphatidylcholine+H₂O 2O=2-acylglycerophosphocholine+a carboxylate. A phospholipases A₁ substrate's specificity may be broader than phospholipase A₂, and typically comprises a Ca²⁺ for improved activity. Phospholipase A₁ producing cells and methods for isolating a phospholipase A₁ from a cellular material and/or a biological source have been described [see, for example, Gatt, S., 1968; van den Bosch, H., et al., 1974; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for a wild-type phospholipase A₁ and/or a functional equivalent amino acid sequence for producing a phospholipase A₁ and/or a functional equivalent include Protein database bank entries: 1FW2, 1FW3, 1ILD, 1ILZ, 1IM0, 1QD5, and/or 1QD6.

g. Phospholipases A₂

Phospholipase A₂ (EC 3.1.1.4) has been also referred to in that art as “phosphatidylcholine 2-acylhydrolase,” “lecithinase A,” “phosphatidase,” and/or “phosphatidolipase,” ad “phospholipase A.” A phospholipase A₂ catalyzes the reaction: phosphatidylcholine+H₂O=1-acylglycerophosphocholine+a carboxylate. A phospholipases A₂ also catalyzes reactions on a phosphatidylethanolamine, a choline plasmalogen and/or a phosphatide, and/or acts on a 2-position ester bond. Ca²⁺ generally improves enzyme function. Phospholipase A₂ producing cells and methods for isolating a phospholipase A₂ from a cellular material and/or a biological source have been described, [see, for example, Saito, K. and Hanahan, D. J., 1962; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for a wild-type phospholipase A₂ and/or a functional equivalent amino acid sequence for producing a phospholipase A₂ and/or a functional equivalent include Protein database bank entries: 1A2A, 1A3D, 1A3F, 1AE7, 1AOK, 1AYP, 1B4W, 1BBC, 1BCI, 1BJJ 1BK9, 1BP2, 1BPQ, 1BUN, 1BVM, 1C1J, 1C74, 1CEH, 1CJY, 1CL5 1CLP, 1 DB4, 1 DB5, 1DCY, 1DPY, 1FAZ, 1FDK, 1FE5, 1FX9, 1FXF 1G0Z, 1G2X, 1G4I, 1GH4, 1GMZ, 1GOD, 1GP7, 1HN4, 1IJL, 1IRB 1IT4, 1IT5, 1J1A, 1JIA, 1JLT, 1JQ8, 1JQ9, 1KP4, 1KPM, 1KQU 1KVO, 1KVW, 1KVX, 1KVY, 1L8S, 1LE6, 1LE7, 1LN8, 1LWB, 1M8R 1M8S, 1M8T, 1MF4, 1MG6, 1 MH2, 1 MH7, 1 MH8, 1MKS, 1MKT, 1MKU 1MKV, 1N28, 1N29, 1O2E, 1O3W, 1OQS, 1OWS, 1OXL, 1OXR, 1OYF 1OZ6, 1OZY, 1P2P, 1P7O, 1PA0, 1PC9, 1PIR, 1PIS, 1PO8, 1POA 1POB, 1POC, 1POD, 1POE, 1PP2, 1PPA, 1PSH, 1PSJ, 1PWO, 1Q6V 1Q7A, 1QLL, 1RGB, 1RLW, 1S6B, 1S8G, 1S8H, 1S8I, 1SFV, 1SFW 1SKG, 1SQZ, 1SV3, 1SV9, 1SXK, 1SZ8, 1T37, 1TC8, 1TD7, 1TDV 1TG1, 1TG4, 1TGM, 1TH6, 1TJ9, 1TJK, 1TJQ, 1TK4, 1TP2, 1U4J1U73, 1UNE, 1VAP, 1VIP, 1VKQ, 1VL9, 1XXS, 1XXW, 1Y38, 1Y4L1Y6O, 1Y6P, 1Y75, 1YXH, 1YXL, 1Z76, 1ZL7, 1ZLB, 1ZM6, 1ZR81ZWP, 1ZYX, 2ARM, 2AZY, 2AZZ, 2B00, 2B01, 2B03, 2B04, 2B17 2B96, 2BAX, 2BCH, 2BD1, 2BPP, 2DO2, 2DPZ, 2DV8, 2FNX, 2G58 2GNS, 2H4C, 2I0U, 2NOT, 2O1N, 2OLI, 2OQD, 2OSH, 2OSN, 2OTF 2OTH, 2OUB, 2OYF, 2PB8, 2PHI, 2PMJ, 2PVT, 2PWS, 2PYC, 2Q1P 2QHD, 2QHE, 2QHW, 2QOG, 2QU9, 2QUE, 2QVD, 2RD4, 2ZBH, 3BJW 3BP2, 3CBI, 3P2P, 4BP2, 4P2P, and/or 5P2P.

h. Phosphatidylinositol Deacylases

Phosphatidylinositol deacylase (EC 3.1.1.52) has been also referred to in that art as “1-phosphatidyl-D-myo-inositol 2-acylhydrolase,” “phosphatidylinositol phospholipase A₂,” and/or “phospholipase A2.” A phosphatidylinositol deacylase catalyzes the reaction: 1-phosphatidyl-D-myo-inositol+H₂O=1-acylglycerophosphoinositol+a carboxylate. Phosphatidylinositol deacylase producing cells and methods for isolating a phosphatidylinositol deacylase from a cellular material and/or a biological source have been described, [see, for example, Gray, N. C. C. and Strickland, K. P., 1982; Gray, N. C. C. and Strickland, K. P., 1982; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

i. Phospholipases C

Phospholipase C (EC 3.1.4.3) has been also referred to in that art as “phosphatidylcholine cholinephosphohydrolase,” “lipophosphodiesterase I,” “lecithinase C,” “Clostridium welchii α-toxin,” “Clostridium oedematiens β- and γ-toxins,” “lipophosphodiesterase C,” “phosphatidase C,” “heat-labile hemolysin,” and/or “α-toxin.” A phospholipase C catalyzes the reaction: phosphatidylcholine+H₂O=1,2-diacylglycerol+choline phosphate. A bacterial phospholipase C may have activity against sphingomyelin and phosphatidylinositol. Phospholipase C producing cells and methods for isolating a phospholipase C from a cellular material and/or a biological source have been described [see, for example, Sheiknejad, R. G. and Srivastava, P. N., 1986; Takahashi, T., et al., 1974; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for a wild-type phospholipase C and/or a functional equivalent amino acid sequence for producing a phospholipase C and/or a functional equivalent include Protein database bank entries: 1AH7, 1CA1, 1GYG, 1IHJ, 1OLP, 1P5X, 1P6D, 1P6E, 1QM6, 1QMD, 2FFZ, 2FGN, and/or 2HUC.

j. Phospholipases D

Phospholipase D (EC 3.1.4.4) has been also referred to in that art as “phosphatidylcholine phosphatidohydrolase,” “lipophosphodiesterase II,” “lecithinase D,” and/or“choline phosphatase.” A phospholipase D catalyzes the reaction: phosphatidylcholine+H₂O=choline+a phosphatidate. A phospholipase D may have activity against other phosphatidyl esters. Phospholipase D producing cells and methods for isolating a phospholipase D from a cellular material and/or a biological source have been described, [see, for example, Astrachan, L. 1973; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for a wild-type phospholipase D and/or a functional equivalent amino acid sequence for producing a phospholipase D and/or a functional equivalent include Protein database bank entries: 1F01, 1V0R, 1V0S, 1V0T, 1V0U, 1V0V, 1V0W, 1V0Y, 2ZE4, and/or 2ZE9.

k. Phosphoinositide Phospholipases C

Phosphoinositide phospholipase C (EC 3.1.4.11) has been also referred to in that art as “1-phosphatidyl-1D-myo-inositol-4,5-bisphosphate inositoltrisphosphohydrolase,” “triphosphoinositide phosphodiesterase,” “phosphoinositidase C,” “1-phosphatidylinositol-4,5-bisphosphate phosphodiesterase,” “monophosphatidylinositol phosphodiesterase,” “phosphatidylinositol phospholipase C,” “PI-PLC,” and/or “1-phosphatidyl-D-myo-inositol-4,5-bisphosphate inositoltrisphosphohydrolase.” A phosphoinositide phospholipase C catalyzes the reaction: 1-phosphatidyl-1D-myo-inositol 4,5-bisphosphate+H₂O=1D-myo-inositol 1,4,5-trisphosphate+diacylglycerol. A phosphoinositide phospholipase C may have activity against other phosphatidyl esters. A phosphoinositide phospholipase C producing cells and methods for isolating a phosphoinositide phospholipase C from a cellular material and/or a biological source have been described, [see, for example, Downes, C. P. and Michell, R. H.1981; Rhee, S. G. and Bae, Y. S. 1997; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for a wild-type phosphoinositide phospholipase C and/or a functional equivalent amino acid sequence for producing a phosphoinositide phospholipase C and/or a functional equivalent include Protein database bank entries: 1DJG, 1DJH, 1DJI, 1DJW, 1DJX, 1DJY, 1DJZ, 1HSQ, 1JAD, 1MAI, 1QAS, 1QAT, 1Y0M, 1YWO, 1YWP, 2C5L, 2EOB, 2FCI, 2FJL, 2FJU, 2HSP, 2ISD, 2K2J, 2PLD, 2PLE, and/or 2ZKM.

I. Phosphatidate Phosphatases

Phosphatidate phosphatase (EC 3.1.3.4) has been also referred to in that art as “3-sn-phosphatidate phosphohydrolase,” “phosphatic acid phosphatase,” “acid phosphatidyl phosphatase,” and “phosphatic acid phosphohydrolase.” A phosphatidate phosphatase catalyzes the reaction: 3-sn-phosphatidate+H₂O=a 1,2-diacyl-sn-glycerol+phosphate. A phosphatidate phosphatase may have activity against other phosphatidyl esters. A phosphatidate phosphatase producing cells and methods for isolating a phosphatidate phosphatase from a cellular material and/or a biological source have been described, [see, for example, Smith, S. W., et al., 1957; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

m. Lysophospholipases

Lysophospholipase (EC 3.1.1.5) has been also referred to in that art as “2-lysophosphatidylcholine acylhydrolase,” “lecithinase B,” “lysolecithinase,” “phospholipase B,” “lysophosphatidase,” “lecitholipase,” “phosphatidase B,” “lysophosphatidylcholine hydrolase,” “lysophospholipase A1,” “lysophopholipase L2,” “lysophospholipaseDtransacylase,” “neuropathy target esterase,” “NTE,” “NTE-LysoPLA,” and “NTE-lysophospholipase.” A lysophospholipase catalyzes the reaction: 2-lysophosphatidylcholine+H₂O=glycerophosphocholine+a carboxylate. Lysophospholipase producing cells and methods for isolating a lysophospholipase from a cellular material and/or a biological source have been described, [see, for example, van den Bosch, H., et al., 1981; van den Bosch, H., et al., 1973; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for a wild-type lysophospholipase and/or a functional equivalent amino acid sequence for producing a lysophospholipase and/or a functional equivalent include Protein database bank entries: 1G86, 1HDK, 1IVN, 1J00, 1JRL, 1LCL, 1QKQ, 1U8U, 1V2G, 2G07, 2G08, 2G09, and/or 2G0A.

n. Sterol Esterases

Sterol esterase (EC 3.1.1.13) has been also referred to in that art as “lysosomal acid lipase,” “sterol esterase,” “cholesterol esterase,” “cholesteryl ester synthase,” “triterpenol esterase,” “cholesteryl esterase,” “cholesteryl ester hydrolase,” “sterol ester hydrolase,” “cholesterol ester hydrolase,” “cholesterase,” and/or “acylcholesterol lipase.” A sterol esterase catalyzes the reaction: steryl ester+H₂O=a sterol+a fatty acid. A sterol esterase may be active against a triglyceride as well. Cholesterol may comprise the substrate used to characterize a sterol esterase, though the enzyme also hydrolyzes a lipid vitamin ester (e.g., vitamin E acetate, vitamin E palmate, vitamin D₃ acetate). A bile salt often activates the enzyme. Sterol esterase producing cells and methods for isolating a sterol esterase from a cellular material and/or a biological source have been described [see, for example, Okawa, Y. and Yamaguchi, T., 1977; via recombinant expression in a baculoviral system in “Methods and Molecular Biology, Volume 109 Lipase and Phospholipase Protocols.” (Mark Doolittle and Karen Reue, Eds.), pp. 177-186, 203-213, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 329-364, 1984.], and may be used in conjunction with the disclosures herein. Structural information for a wild-type sterol esterase and/or a functional equivalent amino acid sequence for producing a sterol esterase and/or a functional equivalent include Protein database bank entries: 1AQL and/or 2BCE.

o. Galactolipases

Galactolipase (EC 3.1.1.26) has been also referred to in that art as “1,2-diacyl-3-β-D-galactosyl-sn-glycerol acylhydrolase,” “galactolipid lipase,” “polygalactolipase,” and/or “galactolipid acylhydrolase.” A galactolipase catalyzes the reaction: 1,2-diacyl-3-β-D-galactosyl-sn-glycerol+2H₂O=3-β-D-galactosyl-sn-glycerol+2 carboxylates. A galactolipase also may have activity against a phospholipid. The substrate for galactolipase comprises a galactolipid abundantly found in plant cells, and organisms that digest plant material (e.g., an animal) also produce this enzyme. Galactolipase producing cells and methods for isolating a galactolipase from a cellular material and/or a biological source have been described, [see, for example, Helmsing, 1969; Hirayama, O., et al., 1975 In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

p. Sphinqomyelin Phosphodiesterases

Sphingomyelin phosphodiesterase (EC 3.1.4.12) has been also referred to in that art as “sphingomyelinase,” “neutral sphingomyelinase,” “sphingomyelin cholinephosphohydrolase,” and/or “sphingomyelin N-acylsphingoosine-hydrolase.” A sphingomyelin phosphodiesterase catalyzes the reaction: sphingomyelin+H₂O═N-acylsphingosine+choline phosphate. A sphingomyelin phosphodiesterase also may have activity against a phospholipid. Sphingomyelin phosphodiesterase producing cells and methods for isolating a sphingomyelin phosphodiesterase from a cellular material and/or a biological source have been described, [see, for example, Chatterjee, S, and Ghosh, N. 1989; Kanfer, J. N., et al., 1966; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

q. Sphingomyelin Phosphodiesterases D

Sphingomyelin phosphodiesterase D (EC 3.1.4.41) has been also referred to in that art as “sphingomyelin ceramide-phosphohydrolase” and/or “sphingomyelinase D.” A sphingomyelin phosphodiesterase D catalyzes the reaction: sphingomyelin+H₂O=ceramide phosphate+choline. A sphingomyelin phosphodiesterase D also may catalyze the reaction: hydrolyses 2-lysophosphatidylcholine to choline and 2-lysophosphatidate. Sphingomyelin phosphodiesterase D producing cells and methods for isolating a sphingomyelin phosphodiesterase D from a cellular material and/or a biological source have been described, [see, for example, Soucek, A. et al., 1971; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

r. Ceramidases

Ceramidase (EC 3.5.1.23) has been also referred to in that art as “N-acylsphingosine amidohydrolase,” “acylsphingosine deacylase,” and or “glycosphingolipid ceramide deacylase sphingomyelin.” A ceramidase catalyzes the reaction: N-acylsphingosine+H₂O=a carboxylate+sphingosine. Ceramidase producing cells and methods for isolating a ceramidase from a cellular material and/or a biological source have been described [see, for example, E. and Gatt, S., 1969; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

s. Wax-Ester Hydrolases

Wax-ester hydrolase (EC 3.1.1.50) has been also referred to in that art as “wax-ester acylhydrolase,” and “jojoba wax esterase,” and/or “WEH.” A wax-ester hydrolase catalyzes the reaction: wax ester+H₂O=a long-chain alcohol+a long-chain carboxylate. A wax-ester hydrolase may also hydrolyze a long-chain acylglycerol. Wax-ester hydrolase producing cells and methods for isolating a wax-ester hydrolase from a cellular material and/or a biological source have been described, [see, for example, Huang, A. H. C. et al., 1978; Moreau, R. A. and Huang, A. H. C., 1981; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

t. Fatty-Acyl-Ethyl-Ester Synthases

Fatty-acyl-ethyl-ester synthase (EC 3.1.1.67) has been also referred to in that art as “long-chain-fatty-acyl-ethyl-ester acylhydrolase,” and/or “FAEES.” A fatty-acyl-ethyl-ester synthase catalyzes the reaction: long-chain-fatty-acyl ethyl ester+H₂O=a long-chain-fatty acid+ethanol. Fatty-acyl-ethyl-ester synthase producing cells and methods for isolating a fatty-acyl-ethyl-ester synthase from a cellular material and/or a biological source have been described [see, for example, Mogelson, S, and Lange, L. G. 1984; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

u. Retinyl-Palmitate Esterases

Retinyl-palmitate esterase (EC 3.1.1.21) has been also referred to in that art as “retinyl-palmitate palmitohydrolase,” “retinyl palmitate hydrolase,” “retinyl palmitate hydrolyase,” and/or “retinyl ester hydrolase.” A retinyl-palmitate esterase catalyzes the reaction: retinyl palmitate+H₂O=retinol+palmitate. A retinyl-palmitate esterase may also hydrolyze a long-chain acylglycerol. Retinyl-palmitate esterase producing cells and methods for isolating a retinyl-palmitate esterase from a cellular material and/or a biological source have been described, [see, for example, T. et al., 2005; Gao, J. and Simon, 2005; Brockerhoff, Hans and Jensen, Robert G. “Lipolytic Enzymes,” 1974], and may be used in conjunction with the disclosures herein.

v. 11-cis-Retinyl-Palmitate Hydrolases

11-cis-retinyl-palmitate hydrolase (EC 3.1.1.63) has been also referred to in that art as “11-cis-retinyl-palmitate acylhydrolase,” “11-cis-retinol palmitate esterase,” and/or “RPH.” An 11-cis-retinyl-palmitate hydrolase catalyzes the reaction: 11-cis-retinyl palmitate+H₂O=11-cis-retinol+palmitate. 11-cis-retinyl-palmitate hydrolase producing cells and methods for isolating a 11-cis-retinyl-palmitate hydrolase from a cellular material and/or a biological source have been described, [see, for example, Blaner, W. S., et al., 1987; Blaner, W. S., et al., 1984; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

w. All-trans-Retinyl-Palmitate Hydrolases

All-trans-retinyl-palmitate hydrolase (EC 3.1.1.64) has been also referred to in that art as “all-trans-retinyl-palmitate acylhydrolase.” All-trans-retinyl-palmitate hydrolase catalyzes the reaction: all-trans-retinyl palmitate+H₂O=all-trans-retinol+palmitate. A detergent generally promotes this enzyme's activity. All-trans-retinyl-palmitate hydrolase producing cells and methods for isolating an All-trans-retinyl-palmitate hydrolase from a cellular material and/or a biological source have been described, [see, for example, Blaner, W. S., Das, et al., 1987; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

x. Cutinases

Cutinase (EC 3.1.1.74) has been also referred to in that art as “cutin hydrolase.” A cutinase catalyzes the reaction: cutin+H₂O=cutin monomers. A cutinase also has lipase and/or carboxylesterase activity noted for not using interfacial activation. Cutinase producing cells and methods for isolating a cutinase from a cellular material and/or a biological source have been described, [see, for example, Garcia-Lepe, R., et al., 1997; Purdy, R. E. and Kolattukudy, P. E., 1975; Sebastian, J., and Kolattukudy, P. E., 1988;

In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 471-504, 1984], and may be used in conjunction with the disclosures herein.

y. Acyloxyacyl Hydrolases

An acyloxyacyl hydrolase (EC 3.1.1.77) catalyzes the reaction: 3-(acyloxy)acyl group of bacterial toxin=3-hydroxyacyl group of bacterial toxin+a fatty acid. An acyloxyacyl hydrolase generally prefers a lipopolysaccharide from a Salmonella typhimurium and related organisms. However, an acyloxyacyl hydrolase may also possess a phospholipase, an acyltransferase, a phospholipase A₂, a lysophospholipase, a phospholipase A₁, a phosphatidylinositol deacylase, a diacylglycerol lipase, and/or a phosphatidyl lipase activity. An acyloxyacyl hydrolase generally prefers saturated C₁₂-C₁₆ fatty acid esters. Acyloxyacyl hydrolase producing cells and methods for isolating an acyloxyacyl hydrolase from a cellular material and/or a biological source have been described, [see, for example, Hagen, F. S., et al., 1991; Munford, R. S, and Hunter, J. P., 1992; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

z. Petroleum Lipolytic Enzymes

A petroleum hydrocarbon generally comprises a mixture of an alkane, a cycloalkane, an aromatic hydrocarbons, and/or a polycyclic aromatic hydrocarbon. This type of lipid differ from a lipid typically catalyzed by an alpha/beta hydrolase, in that a petroleum hydrocarbon lacks a chemical moiety such as an alcohol, an ester bond, and/or a carboxylic acid. Some microorganisms are capable of digesting one or more petroleum lipids, generally by adding one or more oxygen moiety(s) prior to integration of the lipid into cellular metabolic pathways. Often petroleum degradation occurs via a metabolic pathway comprising numerous enzymes and proteins, in some cases bound to various cellular membranes. Such an enzyme and/or a series of enzyme(s) and/or protein(s) that improves a petroleum hydrocarbon's solubility; absorption into a material formulation, etc., may be known herein as a “petroleum lipolytic enzyme” to differentiate it from a lipolytic enzyme that acts on a non-petroleum substrate described herein.

A biomolecular composition may be prepared from a cell and/or a virus that produces such a petroleum lipolytic enzyme. A type of petroleum lipolytic enzyme comprises one that first adds, rather than modifies, a polar solvent solubility enhancing moiety (e.g., an alcohol, an acid), as that initial modification in a degradation pathway may be sufficient to improve solubility and/or an absorptive property of a target petroleum lipid. As exemplified by the Pseudomonas putida alkane degradation pathway encoded by an alkBFGHIJKL operon, a petroleum alkane substrate undergoes catalysis by a plurality of enzymes and/or proteins (e.g., an alkane hydroxylase, a rubredoxins, an aldehyde dehydrogenase, an alcohol dehydrogenase, an acyl-CoA synthetase) and proteins (e.g., an outer membrane protein, a methyl-accepting transducer protein), that convert the alkane into an aldehyde and an acid with the participation of additional enzymes and proteins not encoded by the operon. A membrane bound monooxygenase, a rubredioxin, and a soluble rubredioxin add an alcohol moiety to the petroleum alkane by shunting electrons through a NADH compound to a hydroxylase. These initial enzymatic activities that result in improvement of solubility by addition of an alcohol may be used to select an enzyme. The alcohol may be further catalyzed into an aldehyde, then an acid, before entering regular cellular metabolic pathways (e.g., energy production). Other pathways are thought to use a dioxygenase to initially produce a n-alkyl hydroperoxide that may be converted into an aldehyde, using a flavin adenine dinucleotide, but not a NADPH or a rubredoxin (Van Hamme, J. D., 2003).

Another example of petroleum degradation comprises a polycyclic aromatic hydrocarbon having oxygenated moiety(s) added by the enzymes and proteins expressed from the nahAaAbAcAdBFCED operon for naphthalene degradation. These enzymes and proteins include: a reductase (nahAa), a ferredoxin (nahAb), an iron sulfur protein large subunit (nahAc), an iron sulfur protein small subunit (nahAd), a cis-naphthalene dihydrodiol dehydrogenase (nahB), a salicyaldehyde dehydrogenase (nahF), a 1,2-dihydroxynaphthalene oxygenase (nahC), a 2-hydroxybenzalpyruvate aldolase (nahE), a 2-hydroxychromene-2-carboxylate isomerase (nahD). The nahAa to nahAd genes encode a naphthalene dioxygenase. Pseudomonas putida strains may also have the salicylate degradation pathway, which includes the following enzymes: a salicylate hydroxylase (nahG), a chloroplast-type ferredoxin (nahT), a catechol oxygenase (nahH), a 2-hydroxymuconic semialdehyde dehydrogenase (nahI), a 2-hydroxymuconic semialdehyde dehydrogenase (nahN), a 2-oxo-4-pentenoate hydratase (nahL), a 4-hydroxy-2-oxovalerate aldolase (nahO), an acetaldehyde dehydrogenase (nahM), a 4-oxalocrotonate decarboxylase (nahK), and/or a 2-hydroxymuconate tautomerase (nahJ). Both operons are regulated by salicylate induction of the nahR gene from another operon (Van Hamme, J. D., 2003).

As a petroleum often comprises a mixture of various linear and cyclical hydrocarbons, a plurality of petroleum lipolytic enzymes in a biomolecular composition (e.g., a plurality of cells that act one or more petroleum substrates, a plurality of semipurified or purified petroleum lipolytic enzymes, etc.) are contemplated to act on the petroleum such as to improve the solubility of many or all components of the petroleum. In some embodiments, conversion of the petroleum may occur through a plurality of the steps of a petroleum degradation pathway (e.g., via a cell-based composition comprising the degradation pathway's enzymes).

2. Phosphoric Triester Hydrolases

A material formulation (e.g., a biomolecular composition) may comprise a lipolytic, a petroleum lipolytic enzyme, another enzyme, or a combination thereof. In some embodiments, a lipolytic enzyme may be combined with another enzyme that either does not possess lipolytic activity or has such activity as an additional function, for the purpose to confer an additional catalytic and/or binding property to a material formulation. In certain embodiments, the additional enzyme comprises a hydrolase. An additional hydrolase may comprise an esterase. A type of an additional esterase comprises an esterase that catalyzes the hydrolysis of an organophosphorus compound. Examples of such an additional esterase include those identified by enzyme commission number EC 3.1.8, the phosphoric triester hydrolases. A phosphoric triester hydrolase catalyzes the hydrolytic cleavage of an ester from a phosphorus moiety. Examples of a phosphoric triester hydrolase include an aryldialkylphosphatase (EC 3.1.8.1), a diisopropyl-fluorophosphatase (EC 3.1.8.2), or a combination thereof. A material formulation with multiple biomolecule activities such as a dual enzymatic function (e.g., ease of lipid and organophosphorus compound removal/detoxification), may be of benefit depending upon the type of compounds that contact and/or are comprised as part of such an item.

Examples of a phosphoric triester hydrolase and a cleaved OP compound and a bond type are shown at Table 1.

TABLE 1
Phosphoric Triester Hydrolases

| | OP Compound Phosphoryl Bond-Type and | | | | |
|---|---|---|---|---|---|
| | Phosphoryl Bond Types Cleaved by Enzyme | | | | |
| | | Various OP | Sarin, | VX, | |
| | | Pesticides | Soman | R-VX | Tabun |
| Enzyme | P—C | P—O | P—F | P—S | P—CN |
| OPH^{a,b,c,d,e,f,g} | − | + | + | + | + |
| Human | + | + | + | − | + |
| Paraoxonase^{h,i,j} | | | | | |
| OPAA-2^{k,l} | − | + | + | − | + |
| Squid DFPase^{m} | − | − | + | − | − |

^{a}Dumas, D. P. et al., 1989a;
^{b}Dumas, D. P. et al., 1989b;
^{c}Dumas, D. P. et al., 1990;
^{d}Dave, K. I. et al., 1993;
^{e}Chae, M. Y. et al., 1994;
^{f}Lai, K. et al., 1995;
^{g}Kolakowski, J. E. et al., 1997;
^{h}Hassett, C. et al., 1991;
ⁱJosse, D. et al., 2001;
^{j}Josse, D. et al., 1999;
^{k}DeFrank, J. J. et al. 1993;
^{l}Cheng, T.-C. et al., 1996;
^{m}Hoskin, F. C. G. and Roush, A. H., 1982.

An “organophosphorus compound” comprises a phosphoryl center, and further comprises two or three ester linkages. In some aspects, the type of phosphoester bond and/or additional covalent bond at the phosphoryl center classifies an organophosphorus compound. In embodiments wherein the phosphorus comprises a linkage to an oxygen by a double bond (P═O), the OP compound may be known as an “oxon OP compound” and/or “oxon organophosphorus compound.” In embodiments wherein the phosphorus comprises a linkage to a sulfur by a double bond (P═S), the OP compound may be known as a “thion OP compound” and/or “thion organophosphorus compound.” Additional examples of bond-type classified OP compounds include a phosphonocyanate, which comprises a P—CN bond; a phosphoroamidate, which comprises a P—N bond; a phosphotriester, which comprises a P—O bond; a phosphodiester, which comprises a P═O bond; a phosphonofluoridate, which comprises a P—F bond; and a phosphonothiolate, which comprises a P—S bond. A “dimethyl OP compound” comprises two methyl moieties covalently bonded to the phosphorus atom, such as, for example, a malathion. A “diethyl OP compound” comprises two ethoxy moieties covalently bonded to the phosphorus atom, such as, for example, a diazinon.

In general embodiments, an OP compound comprises an organophosphorus nerve agent and/or an organophosphorus pesticide. As used herein, a “nerve agent” functions as an inhibitor of a cholinesterase, including but not limited to, an acetyl cholinesterase, a butyl cholinesterase, or a combination thereof. The toxicity of an OP compound depends on the rate of release of its phosphoryl center (e.g., P—C, P—O, P—F, P—S, P—CN) from the target enzyme (Millard, C. B. et al., 1999). In specific embodiments, a nerve agent comprises an inhibitor of a cholinesterase (e.g., acetyl cholinesterase) whose catalytic activity may be used for health and survival in an animal, including a human.

Certain OP compounds are so toxic to humans that they have been adapted for use as chemical warfare agents, such as a tabun, a soman, a sarin, a cyclosarin, a GX, and/or a VX (e.g., a R—VX). A CWA may comprise an airborne form and such a formulation may be known herein as an “OP-nerve gas.” Examples of an airborne form include a gas, a vapor, an aerosol, a dust, or a combination thereof. Examples of an OP compound that may be formulated as an OP nerve gas include a tabun, a sarin, a soman, a VX, a cyclosarin, a GX, or a combination thereof.

In addition to the initial inhalation route of exposure common to such an agent, a CWA such as a persistent agent (e.g., a VX, a thickened soman), pose a threat through dermal absorption [In “Chemical Warfare Agents: Toxicity at Low Levels,” (Satu M. Somani and James A. Romano, Jr., Eds.) p. 414, 2001]. A “persistent agent” comprises a CWA formulated [e.g., comprising a thickener such as one or more carbon based polymer(s)] to be less volatile (e.g., non-volatile) and thus remain as a solid and/or liquid (e.g., remain upon a contaminated surface) while exposed to the open air for more than about three hours. Often after release, a persistent agent may convert from an airborne dispersal form to a solid and/or liquid residue on a surface, thus providing the opportunity to contact the skin of a human and/or other target. The toxicities for common OP chemical warfare agents after contact with skin are shown at Table 2.

TABLE 2
LD₅₀ Values* of Common Organophosphorus
Chemical Warfare Agents

| Common OP | Estimated human LD₅₀ - percutaneous |
|---|---|
| CWA | (skin) administration |
| Tabun | 1000 milligrams (“mg”) |
| Sarin | 1700 mg |
| Soman | 100 mg |
| VX | 10 mg |

*LD₅₀ - the dose to kill 50% of individuals in a population after administration, wherein the individuals weigh approximately 70 kg.

In some embodiments, an OP compound may comprise a particularly poisonous organophosphorus nerve agent. A “particularly poisonous” agent possesses a LD₅₀ of 35 mg/kg or less for an organism after percutaneous (“skin”) administration of the agent. Examples of a particularly poisonous OP nerve agent include a tabun, a sarin, a cyclosarin, a soman, a VX, a R—VX, or a combination thereof.

A terms such as “detoxification,” “detoxify,” “detoxified,” “degradation,” “degrade,” and/or “degraded” refers to a chemical reaction of a compound that produces a chemical product less harmful to the health and/or survival of a target organism contacted with the chemical product relative to contact with the parent compound. OP compounds may be detoxified using chemical hydrolysis and/or through enzymatic hydrolysis (Yang, Y.-C. et al., 1992; Yang, Y.-C. et al., 1996; Yang, Y.-C. et al., 1990; LeJeune, K. E. et al., 1998a). In general embodiments, the enzymatic hydrolysis comprises a specifically targeted reaction wherein the OP compound may be cleaved at the phosphoryl center's chemical bond resulting in predictable products that are acidic in nature but benign from a neurotoxicity perspective (Kolakowski, J. E. et al., 1997; Rastogi, V. K. et al., 1997; Dumas, D. P. et al., 1990; Raveh, L. et al., 1992). By comparison, chemical hydrolysis may be much less specific, and in the case of VX may produce some quantity of byproducts that approach the toxicity of the intact agent (Yang, Y.-C. et al., 1996; Yang, Y.-C. et al., 1990). In facets, an enzyme composition degrades a CWA, a particularly poisonous organophosphorus nerve agent, or a combination thereof, into product that may be not particularly poisonous.

Many OP compounds are pesticides that are not particularly poisonous to a human, though they do possess varying degrees of toxicity to a human and/or another animal. Examples of an OP pesticide include a bromophos-ethyl, a chlorpyrifos, a chlorfenvinphos, a chlorothiophos, a chlorpyrifos-methyl, a coumaphos, a crotoxyphos, a crufomate, a cyanophos, a diazinon, a dichlofenthion, a dichlorvos, a dursban, an EPN, an ethoprop, an ethyl-parathion, an etrimifos, a famphur, a fensulfothion, a fenthion, a fenthrothion, an isofenphos, a jodfenphos, a leptophos-oxon, a malathion, a methyl-parathion, a mevinphos, a paraoxon, a parathion, a parathion-methyl, a pirimiphos-ethyl, a pirimiphos-methyl, a pyrazophos, a quinalphos, a ronnel, a sulfopros, a sulfotepp, a trichloronate, or a combination thereof. In some embodiments, a composition degrades a pesticide into a product that may be less toxic to an organism. In specific aspects, the organism comprises an animal, such as a human.

a. Aryldialkylphosphatases

An aryldialkylphosphatase (EC 3.1.8.1) may be also known by its systemic name “aryltriphosphate dialkylphosphohydrolase” and various enzymes in this category have been known in the art by names such as “organophosphate hydrolase”; “paraoxonase”; “A-esterase”; “aryltriphosphatase”; “organophosphate esterase”; “esterase B1”; “esterase E4”; “paraoxon esterase”; “pirimiphos-methyloxon esterase”; “OPA anhydrase”; “organophosphorus hydrolase”; “phosphotriesterase”; “PTE”; “paraoxon hydrolase”; “OPH”; and/or “organophosphorus acid anhydrase.” An aryldialkylphosphatase catalyzes the following reaction: aryl dialkyl phosphate+H₂O=an aryl alcohol+dialkyl phosphate. Examples of an aryl dialkyl phosphate include an organophosphorus compound comprising a phosphonic acid ester, a phosphinic acid ester, or a combination thereof. Aryldialkylphosphatase producing cells and methods for isolating an aryldialkylphosphatase from a cellular material and/or a biological source have been described, [see, for example, Bosmann, H. B., 1972; and Mackness, M. I. et al., 1987.], and may be used in conjunction with the disclosures herein. Structural information for a wild-type aryldialkylphosphatase and/or a functional equivalent amino acid sequence for producing an aryldialkylphosphatase and/or a functional equivalent include Protein database bank entries: 1EYW, 1EZ2, 1 HZY, 1I0B, 1I0D, 1JGM, 1P6B, 1P6C, 1P9E, 1QW7, 1V04, 2D2G, 2D2H, 2D2J, 2O4M, 2O4Q, 2OB3, 2OQL, 2R1K, 2R1L, 2R1M, 2R1N, 2R1P, 2VC5, 2VC7, 2ZC1, 3C86, 3CAK, and/or 3E3H. Examples of an aryldialkylphosphatase and/or a functional equivalent KEEG sequences for production of wild-type and/or a functional equivalent nucleotide and protein sequence include: HSA-5444(PON1), 5445(PON2), 5446(PON3); PTR-463547(PON1), 463548(PON3), 463549(PON2); MCC-699107, 699236, 699355(PON1); MMU-18979(Pon1), 269823(Pon3), 330260(Pon2); RNO-296851(Pon2), 84024(Pon1); CFA-403855(PON2); BTA-281417(PON2); SSC-100048952(PON1), 100142663(PON2), 733674(PON3); MDO-100017970; GGA-395830(PON2); SPU-582780; MBO-Mb0235c(php); MBB-BCG_{—}0267c(php); MMC-Mmcs_{—}0224; MKM-Mkms_{—}0234; MJL-Mjls_{—}0214; and/or RXY-Rxyl_{—}2340.

i. Organophosphorus Hydrolases

Organophosphorus hydrolase (E.C.3.1.8.1) has been also referred to in that art as “organophosphate-hydrolyzing enzyme,” “phosphotriesterase,” “PTE,” “organophosphate-degrading enzyme,” “OP anhydrolase,” “OP hydrolase,” “OP thiolesterase,” “organophosphorus triesterase,” “parathion hydrolase,” “paraoxonase,” “DFPase,” “somanase,” “VXase,” and/or “sarinase.” As used herein, this type of enzyme may be referred to herein as “organophosphorus hydrolase” and/or “OPH.”

The initial discovery of OPH was from two bacterial strains from the closely related genera: Pseudomonas diminuta and Flavobacterium spp. (McDaniel, S. et al., 1988; Harper, L. et al., 1988), which encoded identical organophosphorus degrading opd genes on plasmids (Genbank accession no. M20392 and Genbank accession no. M22863) (copending U.S. patent application Ser. No. 07/898,973, incorporated herein in its entirety by reference). The Pseudomonas diminuta may have been derived from the Flavobacterium spp. Subsequently, other OPH encoding genes have been discovered. The use of any opd gene and/or the gene product in the described compositions, articles, methods, etc. is contemplated. Examples of an opd gene and a gene product that may be used include an Agrobacterium radiobacter P230 organophosphate hydrolase gene, opdA (Genbank accession no. AY043245; Entrez databank no. AAK85308); a Flavobacterium balustinum opd gene for parathion hydrolase (Genbank accession no. AJ426-431; Entrez databank no. CAD19996); a Pseudomonas diminuta phosphodiesterase opd gene (Genbank accession no. M20392; Entrez databank no. AAA98299; Protein Data Bank entries 1JGM, 1DPM, 1EYW, 1EZ2, 1 HZY, 1108, 1IOD, 1PSC and 1PTA); a Flavobacterium sp opd gene (Genbank accession no. M22863; Entrez databank no. AAA24931; ATCC 27551); a Flavobacterium sp. parathion hydrolase opd gene (Genbank accession no. M29593; Entrez databank no. AAA24930; ATCC 27551); or a combination thereof (Horne, I. et al., 2002; Somara, S. et al., 2002; McDaniel, C. S. et al., 1988a; Harper, L. L. et al., 1988; Mulbry, W. W. and Karns, J. S., 1989).

Because OPH possesses the property of cleaving a broad range of OP compounds (Table 1), the OP detoxifying enzyme that has been often studied and characterized, with the enzyme obtained from Pseudomonas being the target of focus for many studies. This OPH was initially purified following expression from a recombinant baculoviral vector in insect tissue culture of the Fall Armyworm, Spodoptera frugiperda (Dumas, D. P. et al., 1989b). Purified enzyme preparations have been shown to be able to detoxify via hydrolysis a wide spectrum of structurally related insect and mammalian neurotoxins that function as an acetylcholinesterase inhibitor. Of great interest, this detoxification ability included a number of organophosphorofluoridate nerve agents such as a sarin and a soman. This was the first recombinant DNA construction encoding an enzyme capable of degrading these nerve gases. This enzyme was capable of degrading the common organophosphorus insecticide analog (paraoxon) at rates exceeding 2×10⁷ M⁻¹ (mole enzyme)⁻¹, which may be equivalent to the catalytically efficient enzymes observed in nature. The purified enzyme preparations are capable of detoxifying a sarin and the less toxic model mammalian neurotoxin O,O-diisopropyl phosphorofluoridate (“DFP”) at the equivalent rates of 50-60 molecules per molecule of enzyme-dimer per second. In addition, the enzyme may hydrolyze a soman and a VX at approximately 10% and 1% of the rate of a sarin, respectively. The breadth of substrate utility (e.g., a V agent, a sarin, a soman, a tabun, a cyclosarin, an OP pesticide) and the efficiency for the hydrolysis exceeds the known abilities of other prokaryotic and eukaryotic organophosphorus acid anhydrases, and this detoxification may be due to a single enzyme rather than a family of related, substrate-limited proteins.

The X-ray crystal structure of Pseudomonas OPH has been determined (Benning, M. M. et al., 1994; Benning, M. M. et al., 1995; Vanhooke, J. L. et al., 1996). An OPH monomer's active site binds two atoms of Zn²⁺; however, OPH may be prepared wherein Co²⁺ replaces Zn²⁺, which enhances catalytic rates. Examples of the catalytic rates (k_{cat}) and specificities (k_{cat}/kₘ) for Co²⁺ substituted OPH against various OP compounds are shown at Table 3 below.

TABLE 3
Catalytic Activity of Wild-Type OPH binding Co²⁺

| | k_{cat} (s⁻¹) | k_{cat}/Kₘ (M⁻¹ s⁻¹) |
|---|---|---|
| OP Pesticide Substrate | | |
| Paraoxon | 15000^{a} | 1.3 × 10⁸ |
| OP CWA Substrates | | |
| Sarin | 56^{b} | 8 × 10⁴ |
| Soman | 5^{b} | 1 × 10⁴ |
| VX | 0.3^{b} | 7.5 × 10² |
| R-VX | 0.5^{c} | 105 |
| Tabun* | 77^{d} | 7.6 × 10⁵ |

*Wild-type Zn²⁺ OPH was used in obtaining these kinetic parameters;
^{a}diSioudi, B. et al., 1999a;
^{b}Kolakoski, J. E. et al., 1997;
^{c}Rastogi, V. K. et al., 1997;
^{d}Raveh, L. et al., 1992.

The phosphoryl center of OP compounds is chiral, and Pseudomonas OPH preferentially binds and/or cleaves Sₚ enantiomers over Rₚ enantiomers of the chiral phosphorus in various substrates by a ratio of about 10:1 to about 90:1 (Chen-Goodspeed, M. et al., 2001a; Hong, S.-B. and Raushel, F. M., 1999a; Hong, S.-B. and Raushel, F. M., 1999b). A CWA such as a VX, a sarin, and/or a soman are usually prepared and used as a mixture of sterioisomers of varying toxicity, with VX and sarin having two enantiomers each, with the chiral center around the phosphorus of the cleavable bond. Soman possesses four enantiomers, with one chiral center based on the phosphorus and an additional chiral center based on a pinacolyl moiety [In “Chemical Warfare Agents: Toxicity at Low Levels” (Satu M. Somani and James A. Romano, Jr., Eds.) pp 26-29, 2001; Li, W.-S. et al., 2001; Yang, Y.-C. et al., 1992; Benshop, H. P. et al., 1988]. The Sₚ enantiomer of sarin may be about 10⁴ times faster in inactivating acetylcholinesterase than the Rₚ enantiomer (Benschop, H. P. and De Jong, L. P. A. 1988), while the two Sₚ enantiomers of soman may be about 10⁵ times faster in inactivating acetylcholinesterase than the Rₚ enantiomers (Li, W.-S. et al., 2001; Benschop, H. P. et al., 1984). Wild-type organophosphorus hydrolase seems to have greater specificity for the less toxic enantiomers of sarin and soman. OPH may be about 9-fold faster cleaving an analog of the Rₚ enantiomer of sarin relative to an analog of the Sₚ enantiomer, and about 10-fold faster in cleaving analogs of the R_{c} enantiomers of soman relative to analogs of the S_{c} enantiomers (Li, W.-S. et al., 2001).

ii. Paraoxonases

A peraoxonase such as a human paraoxonase (EC 3.1.8.1) comprises a calcium dependent protein, and may be also known as an “arylesterase” and/or “aryl-ester hydrolase” (Josse, D. et al., 1999; Vitarius, J. A. and Sultanos, L. G., 1995). Examples of the human paraoxonase (“HPON1”) gene and gene products may be accessed at (Genbank accession no. M63012; Entrez databank no. AAB59538) (Hassett, C. et al., 1991).

b. Diisopropyl-Fluorophosphatases

A diisopropyl-fluorophosphatase (EC 3.1.8.2) may be also known by its systemic name “diisopropyl-fluorophosphate fluorohydrolase,” and various enzymes in this category have been known in the art by names such as “DFPase”; “tabunase”; “somanase”; “organophosphorus acid anhydrolase”; “organophosphate acid anhydrase”; “OPA anhydrase”; “diisopropylphosphofluoridase”; “dialkylfluorophosphatase”; “diisopropyl phosphorofluoridate hydrolase”; “isopropylphosphorofluoridase”; and/or “diisopropylfluorophosphonate dehalogenase.” A diisopropyl-fluorophosphatase catalyzes the following reaction: diisopropyl fluorophosphate+H₂O=fluoride+diisopropyl phosphate. Examples of a diisopropyl fluorophosphate include an organophosphorus compound comprising a phosphorus-halide, a phosphorus-cyanide, or a combination thereof. Diisopropyl-fluorophosphatase producing cells and methods for isolating a diisopropyl-fluorophosphatase from a cellular material and/or a biological source have been described, [see, for example, Cohen, J. A. and Warring, M. G., 1957], and may be used in conjunction with the disclosures herein. Structural information for a wild-type diisopropyl-fluorophosphatase and/or a functional equivalent amino acid sequence for producing a diisopropyl-fluorophosphatase and/or a functional equivalent include Protein database bank entries: 1E1A, 1PJX, 2GVU, 2GVV, 2GVW, 2GVX, 21A0, 21AP, 2IAQ, 2IAR, 2IAS, 2IAT, 2IAU, 2IAV, 2IAW, 2IAX, 2W43, and/or 3BYC.

i. OPAAs

Organophosphorus acid anhydrolases (E.C.3.1.8.2), known as “OPAAs,” have been isolated from microorganisms and identified as enzymes that detoxify OP compounds (Serdar, C. M. and Gibson, D. T., 1985; Mulbry, W. W. et al., 1986; DeFrank, J. J. and Cheng, T.-C., 1991). The better-characterized OPAAs have been isolated from an Altermonas species, such as an Alteromonas sp JD6.5, an Alteromonas haloplanktis, and an Altermonas undina (ATCC 29660) (Cheng, T.-C. et al., 1996; Cheng, T.-C. et al., 1997; Cheng, T. C. et al., 1999; Cheng, T.-C. et al., 1993). Examples of an OPAA gene and a gene product that may be used include an Alteromonas sp JD6.5 opaA gene, (GeneBank accession no. U29240; Entrez databank no. AAB05590); an Alteromonas haloplanktis prolidase gene (GeneBank accession no. U56398; Entrez databank AAA99824; ATCC 23821); or a combination thereof (Cheng, T. C. et al., 1996; Cheng, T.-C. et al., 1997). The wild-type encoded OPAA from an Alteromonas sp JD6.5 comprises 517 amino acids, while the wild-type encoded OPAA from an Alteromonas haloplanktis comprises 440 amino acids (Cheng, T. C. et al., 1996; Cheng, T.-C. et al., 1997). The Alteromonas OPAAs accelerates the hydrolysis of a phosphotriester and/or a phosphofluoridate, including a cyclosarin, a sarin and/or a soman (Table 4).

TABLE 4
Catalytic Activity of Wild-Type OPAAs

| | k_{cat} (s⁻¹) per species OPAA per OP Substrate | | |
|---|---|---|---|
| | A. sp JD6.5 | A. haloplanktis | A. undina |
| OP Compound Substrate | | | |
| DFP | 1650^{a} | 575^{a} | 1239^{a} |
| OP CWA Substrates | | | |
| Sarin | 611^{a} | 257^{a} | 376^{a} |
| Cyclosarin | 1650^{a} | 269^{a} | 1586^{a} |
| Soman | 3145^{a} | 1389^{a} | 2496^{a} |
| Tabun | 85^{a} | 113^{a} | 292^{a} |

^{a}Cheng, T. C. et al., 1999

Similar to OPH, OPAA from an Alteromonas sp JD6.5 (“OPAA-2”) possesses a general binding and cleavage preference up to 112:1 for the Sₚ enantiomers of various p-nitrophenyl phosphotriesters (Hill, C. M. et al., 2000). Additionally, an OPAA from an Alteromonas sp JD6.5 may be over 2 fold faster at cleaving a Sₚ enantiomer of a sarin analog, and over 15-fold faster in cleaving analogs of the R_{c} enantiomers of soman relative to analogs of the Sₚ enantiomers (Hill, C. M. et al., 2001).

ii. Squid-Type DFPases

A “squid-type DFPase” (EC 3.1.8.2) refers to an enzyme that catalyzes the cleavage of both a DFP and a soman, and may be isolated from organisms of the Loligo genus. Generally, a squid-type DFPase cleaves a DFP at a faster rate than a soman. Squid-type DFPases include, for example, a DFPase obtained from a Loligo vulgaris, a Loligo pealei, a Loligo opalescens, or a combination thereof (Hoskin, F. C. G. et al., 1984; Hoskin, F. C. G. et al., 1993; Garden, J. M. et al., 1975).

A well-characterized example of a squid-type DFPase includes the DFPase that has been isolated from the optical ganglion of a Loligo vulgaris (Hoskin, F. C. G. et al., 1984). This squid-type DFPase cleaves a variety of OP compounds, including a DFP, a sarin, a cyclosarin, a soman, and a tabun (Hartleib, J. and Ruterjans, H., 2001a). The gene encoding this squid-type DFP has been isolated, and may be accessed at GeneBank accession no. AX018860 (International patent publication: WO 9943791-A). Further, this enzyme's X-ray crystal structure has been determined (Protein Data Bank entry 1E1A) (Koepke, J. et al., 2002; Scharff, E. I. et al., 2001). This squid-type DFPase binds two Ca²⁺ ions, which function in catalytic activity and enzyme stability (Hartleib, J. et al., 2001). Both the DFPase from a Loligo vulgaris and a Loligo pealei are susceptible to proteolytic cleavage into a 26-kDa and 16 kDa fragments, and the fragments from a Loligo vulgaris are capable of forming active enzyme when associated together (Hartleib, J. and Ruterjans, H., 2001a).

iii. Mazur-Type DFPases

As used herein, a “Mazur-type DFPase” (EC 3.1.8.2) refers to an enzyme that catalyzes the cleavage of both DFP and soman. Generally, a Mazur-type DFPase cleaves a soman at a faster rate than a DFP. Examples of a Mazur-type DFPase include the DFPase isolated from a mouse liver (Billecke, S. S. et al., 1999), which may be the same as the DFPase known as a SMP-30 (Fujita, T. et al., 1996; Billecke, S. S. et al., 1999; Genebank accession no. U28937; Entrez databank AAC52721); a DFPase isolated from a rat liver (Little, J. S. et al., 1989); a DFPase isolated from a hog kidney; a DFPase isolated from a Bacillus stearothermophilus strain OT; a DFPase isolated from an Escherichia coli (ATCC25922) (Hoskin, F. C. G. et al., 1993; Hoskin, F. C. G, 1985); or a combination thereof.

c. Other Phosphoric Triester Hydrolases

Any phosphoric triester hydrolase known in the art may be used. An example of an additional phosphoric triester hydrolase includes a product of the gene, mpd, (GenBank accession number AF338729; Entrez databank AAK14390) isolated from a Plesiomonas sp. strain M6 (Zhongli, C. et al., 2001). Other examples include a phosphoric triester hydrolase identified in a Xanthomonas sp. (Tchelet, R. et al., 1993); a Tetrahymena (Landis, W. G. et al., 1987); certain plants such as a Myriophyllum aquaticum, Spirodela origorrhiza L, an Elodea Canadensis and a Zea mays (Gao, J. et al., 2000; Edwards, R. and Owen, W. J., 1988); and/or in a hen liver and a brain (Diaz-Alejo, N. et al., 1998).

3. Sulfuric Ester Hydrolases

A sulfuric ester hydrolase (EC 3.1.6) catalyzes the hydrolysis of a sulfuric ester bond. Examples of a sulfuric ester hydrolase include an arylsulfatase (EC 3.1.6.1), a steryl-sulfatase (EC 3.1.6.2), a glycosulfatase (EC 3.1.6.3), a N-acetylgalactosamine-6-sulfatase (EC 3.1.6.4), a choline-sulfatase (EC 3.1.6.6), a cellulose-polysulfatase (EC 3.1.6.7), a cerebroside-sulfatase (EC 3.1.6.8), a chondro-4-sulfatase (EC 3.1.6.9), a chondro-6-sulfatase (EC 3.1.6.10), a disulfoglucosamine-6-sulfatase (EC 3.1.6.11), a N-acetylgalactosamine-4-sulfatase (EC 3.1.6.12), an iduronate-2-sulfatase (EC 3.1.6.13), a N-acetylglucosamine-6-sulfatase (EC 3.1.6.14), a N-sulfoglucosamine-3-sulfatase (EC 3.1.6.15), a monomethyl-sulfatase (EC 3.1.6.16), a D-lactate-2-sulfatase (EC 3.1.6.17), a glucuronate-2-sulfatase (EC 3.1.6.18), or a combination thereof.

a. Arylsulfatases

An example of a sulfuric ester hydrolase includes an arylsulfatase (EC 3.1.6.1), which has been also referred to as “sulfatase,” “nitrocatechol sulfatase,” “phenolsulfatase,” “phenylsulfatase,” “p-nitrophenyl sulfatase,” “arylsulfohydrolase,” “4-methylumbelliferyl sulfatase,” “estrogen sulfatase,” “arylsulfatase C,” “arylsulfatase B,” “arylsulfatase A,” and/or “aryl-sulfate sulfohydrolase.” An arylsulfatase catalyzes the reaction: a phenol sulfate+H2O=a phenol+a sulfate. As with other sulfuric ester hydrolases, arylsulfatase producing cells and methods for isolating an arylsulfatase from a cellular material and/or a biological source have been described, [see, for example, Dodgson, K. S. et al., 1956; Roy, A. B. 1960; Roy, A. B., 1976; Webb, E. C. and Morrow, P. F. W., 1959), and may be used in conjunction with the disclosures herein. Structural information for a wild-type arylsulfatase and/or a functional equivalent amino acid sequence for producing an arylsulfatase and/or a functional equivalent include Protein database bank entries: 1 HDH. Examples of an arylsulfatase and/or a functional equivalent KEEG sequences for production of wild-type and/or a functional equivalent nucleotide and protein sequence include: HSA-414(ARSD), 415(ARSE); MCC-704070, 720575(ARSE); CFA-491718(ARSD), 491719(ARSE); BTA-505899(ARSE); MDO-100010082, 100010127; GGA-418658(ARSD); KLA-KLLA0F03146g; DHA-DEHA0F17710g; YLI -YALI0D26488g; SPO-SPBPB10D8.02c; MGR-MGG_{—}10308; ANI-AN6847.2; AFM-AFUA_{—}5G12940, AFUA_{—}8G02520; AOR-AO090120000416; ANG-An01g06640, An08g08530; CNE-CNC06820; UMA-UM05068.1; ECO-b3801(aslA); ECJ-JW3773(aslA); ECE-Z5314(aslA); ECS-ECs4731; ECC-c4719(aslA); ECI-UT189-C4359(aslA); ECP-ECP_{—}3993; SPQ-SPAB_{—}03892; SEC-SC3062(ars); STM-STM3122; SBC-SbBS512-E4119; SDY-SDY_{—}3945(aslA); WU-VV2_{—}0149, VV2_{—}0151; VVY-VVA0659, VVA0661; VPA-VPA0600, VPA0680, VPA0683; VFI-VF_{—}1427(aslA), VF_{—}1428, VF_{—}1430, VF_A0899, VF_A0992(ydeN); PAE-PA0183(atsA); PAU-PA14_{—}02310(atsA); PPU-PP_{—}3352; PFL-PFL_{—}0205, PFL_{—}2842; PFO-Pfl01_{—}0208; ACI-ACIAD1598(atsA); ACB-A1S_{—}0977; ABM-ABSDF2424(atsA); ABY-ABAYE2815; SSE-Ssed_{—}3990; SHE-Shewmr4_{—}2074; SHM-Shewmr7_{—}1901; CPS-CPS_{—}0660, CPS_{—}0841(atsA), CPS_{—}2983, CPS_{—}2984, CPS_{—}2985, CPS_{—}3032; PAT-Patl_{—}0870; FTU-FTT0783(ars); FTF-FTF0783(ars); REU-Reut_A2893, Reut_B4569; REH-H16_A1602, H16_B0315, H16_B0483; RME-Rmet_{—}5416, Rmet_{—}5423; BXE-Bxe_A2132; BUR-Bcep18194_B2584; BCH-Bcen2424_{—}3543; BPE-BP1635; BPA-BPP2750; BBR-BB2736; MPT-Mpe_A2680; MXA-MXAN_{—}6507; MLO-mll5471; SME-SM_{—}b20915(aslA1), SMa0943; RLE-RL1149, RL1237, RL1238, RL1911, RL1918, RL2264, RL2267; BJA-bll5074(arsA); BBT-BBta_{—}0599, BBta_{—}3535; MEX-Mext_{—}0526; SIL-SPO3286(atsA); RDE-RD1_{—}0531, RD1_{—}3744; DSH-Dshi_{—}0936, Dshi_{—}3111; MTU-R0663(atsD), R3299c(atsB); MTC-MT0692, MT0738(atsA), MT3398; MRA-MRA_{—}0673(atsD), MRA_{—}0719(atsA); MBO-Mb0682(atsD), Mb0731(atsAa), Mb0732(atsAb), Mb3327c(atsB); MBB-BCG_{—}0712(atsD), BCG_{—}0761(atsA), BCG_{—}3328c(atsB), BCG_{—}3364c(atsB_{—}2); MAV-MAV_{—}2989, MAV_{—}4461; MSM-MSMEG_{—}1451; MUL-MUL_{—}0227(aslA), MUL_{—}0454(atsD), MUL_{—}2658(atsB); MVA-Mvan_{—}1317; MMC-Mmcs_{—}1023, Mmcs_{—}3964, Mmcs_{—}4113; MKM-Mkms_{—}1040; MJL-Mjls_{—}1052, Mjls_{—}3978, Mjls_{—}4344; CGL-NCg12422(cg12508); CEF-CE1568; RHA-RHA1_ro02004, RHA1_ro03308, RHA1_ro04570, RHA1_ro05958; SEN-SACE_{—}3101(atsD); STP-Strop_{—}2930; RBA-RB11116(aslA), RB1477(atsA), RB1610(aslA), RB1736, RB2367, RB3876(arsA), RB3877(aslA), RB607, RB684, RB686, RB7772(atsA), RB9498(arsA), RB9530(aslA); AMU-Amuc_{—}0565; AVA-Ava_{—}0111; PMT-PMT1515; PMF-P9303_{—}04271; BTH-BT_{—}3093; BFR-BF0017; BFS-BF0016; FJO-Fjoh_{—}3142, Fjoh_{—}3143, Fjoh_{—}3283, Fjoh_{—}4652; MAC-MA2648(atsA); MBA-Mbar_A3081; MMA-MM_{—}1892; HWA-HQ2428A(aslA), HQ2690A(aslA), HQ3203A(aslA), HQ3464A(aslA), HQ3540A(aslA), HQ3543A; NPH-NP0946A; and/or RCI-RCIX63(atsA.

4. Peptidases

A peptidase catalyzes a reaction on a peptide bond, though other secondary reactions (e.g., an esterase activity) may also be catalyzed in some cases. A peptidase generally may be categorized as either an exopeptidase (EC 3.4.11-19) or an endopeptidase (EC 3.4.21-24 and EC 3.4.99). Examples of a peptidase include an alpha-amino-acyl-peptide hydrolase (EC 3.4.11), a peptidyl-amino-acid hydrolase (EC 3.4.17), a dipeptide hydrolase (EC 3.4.13), a peptidyl peptide hydrolase (EC 3.4), a peptidylamino-acid hydrolase (EC 3.4), an acylamino-acid hydrolase (EC 3.4), an aminopeptidase (EC 3.4.11), a dipeptidase (EC 3.4.13), a dipeptidyl-peptidase (EC 3.4.14), a tripeptidyl-peptidase (EC 3.4.14), a peptidyl-dipeptidase (EC 3.4.15), a serine-type carboxypeptidase (EC 3.4.16), a metallocarboxypeptidase (EC 3.4.17), a cysteine-type carboxypeptidase (EC 3.4.18), an omega peptidase (EC 3.4.19), a serine endopeptidase (EC 3.4.21), a cysteine endopeptidase (EC 3.4.22), an aspartic endopeptidase (EC 3.4.23), a metalloendopeptidase (EC 3.4.24), a threonine endopeptidase (EC 3.4.25), an endopeptidase of unknown catalytic mechanism (EC 3.4.99), or a combination thereof. Examples of a serine endopeptidase (EC 3.4.21) includes a chymotrypsin (EC 3.4.21.1); a chymotrypsin C (EC 3.4.21.2); a metridin (EC 3.4.21.3); a trypsin (EC 3.4.21.4); a thrombin (EC 3.4.21.5); a coagulation factor Xa (EC 3.4.21.6); a plasmin (EC 3.4.21.7); an enteropeptidase (EC 3.4.21.9); an acrosin (EC 3.4.21.10); an α-Lytic endopeptidase (EC 3.4.21.12); a glutamyl endopeptidase (EC 3.4.21.19); a cathepsin G (EC 3.4.21.20); a coagulation factor Vila (EC 3.4.21.21); a coagulation factor IXa (EC 3.4.21.22); a cucumisin (EC 3.4.21.25); a prolyl oligopeptidase (EC 3.4.21.26); a coagulation factor Xla (EC 3.4.21.27); a brachyurin (EC 3.4.21.32); a plasma kallikrein (EC 3.4.21.34); a tissue kallikrein (EC 3.4.21.35); a pancreatic elastase (EC 3.4.21.36); a leukocyte elastase (EC 3.4.21.37); a coagulation factor XIIa (EC 3.4.21.38); a chymase (EC 3.4.21.39); a complement subcomponent C (EC 3.4.21.41); a complement subcomponent C (EC 3.4.21.42); a classical-complement-pathway C3/C5 convertase (EC 3.4.21.43); a complement factor I (EC 3.4.21.45); a complement factor D (EC 3.4.21.46); an alternative-complement-pathway C3/C5 convertase (EC 3.4.21.47); a cerevisin (EC 3.4.21.48); a hypodermin C (EC 3.4.21.49); a lysyl endopeptidase (EC 3.4.21.50); an endopeptidase La (EC 3.4.21.53); a γ-renin (EC 3.4.21.54); a venombin AB (EC 3.4.21.55); a leucyl endopeptidase (EC 3.4.21.57); a tryptase (EC 3.4.21.59); a scutelarin (EC 3.4.21.60); a kexin (EC 3.4.21.61); a subtilisin (EC 3.4.21.62); an oryzin (EC 3.4.21.63); a peptidase K (EC 3.4.21.64); a thermomycolin (EC 3.4.21.65); a thermitase (EC 3.4.21.66); an endopeptidase So (EC 3.4.21.67); a t-plasminogen activator (EC 3.4.21.68); a protein C (activated) (EC 3.4.21.69); a pancreatic endopeptidase E (EC 3.4.21.70); a pancreatic elastase 11 (EC 3.4.21.71); an IgA-specific serine endopeptidase (EC 3.4.21.72); a u-plasminogen activator (EC 3.4.21.73); a venombin A (EC 3.4.21.74); a furin (EC 3.4.21.75); a myeloblastin (EC 3.4.21.76); a semenogelase (EC 3.4.21.77); a granzyme A (EC 3.4.21.78); a granzyme B (EC 3.4.21.79); a streptogrisin A (EC 3.4.21.80); a streptogrisin B (EC 3.4.21.81); a glutamyl endopeptidase II (EC 3.4.21.82); an oligopeptidase B (EC 3.4.21.83); a limulus clotting factor (EC 3.4.21.84); a limulus clotting factor (EC 3.4.21.85); a limulus clotting enzyme (EC 3.4.21.86); a repressor LexA (EC 3.4.21.88); a signal peptidase I (EC 3.4.21.89); a togavirin (EC 3.4.21.90); a flavivirin (EC 3.4.21.91); an endopeptidase Clp (EC 3.4.21.92); a proprotein convertase 1 (EC 3.4.21.93); a proprotein convertase 2 (EC 3.4.21.94); a snake venom factor V activator (EC 3.4.21.95); a lactocepin (EC 3.4.21.96); an assemblin (EC 3.4.21.97); a hepacivirin (EC 3.4.21.98); a spermosin (EC 3.4.21.99); a sedolisin (EC 3.4.21.100); a xanthomonalisin (EC 3.4.21.101); a C-terminal processing peptidase (EC 3.4.21.102); a physarolisin (EC 3.4.21.103); a mannan-binding lectin-associated serine protease-2 (EC 3.4.21.104); a rhomboid protease (EC 3.4.21.105); a hepsin (EC 3.4.21.106); a peptidase Do (EC 3.4.21.107); a HtrA2 peptidase (EC 3.4.21.108); a matriptase (EC 3.4.21.109); a C5a peptidase (EC 3.4.21.110); an aqualysin 1 (EC 3.4.21.111); a site-1 protease (EC 3.4.21.112); a pestivirus NS3 polyprotein peptidase (EC 3.4.21.113); an equine arterivirus serine peptidase (EC 3.4.21.114); an infectious pancreatic necrosis birnavirus Vp4 peptidase (EC 3.4.21.115); a SpolyB peptidase (EC 3.4.21.116); a stratum corneum chymotryptic enzyme (EC 3.4.21.117); a kallikrein 8 (EC 3.4.21.118); a kallikrein 13 (EC 3.4.21.119); an oviductin (EC 3.4.21.120); or a combination thereof.

a. Trypsins

Trypsin (EC 3.4.21.4; CAS registry number: 9002-07-7) has been also referred to in that art as “α-trypsin,” “β-trypsin,” “cocoonase,” “parenzyme,” “parenzymol,” “tryptar,” “trypure,” “pseudotrypsin,” “tryptase,” “tripcellim,” and/or “sperm receptor hydrolase.” A trypsin catalyzes the reaction: a preferential cleavage at an Arg and/or a Lys residue. Trypsin producing cells and methods for isolating a trypsin from a cellular material and/or a biological source have been described [see, for example, Huber, R. and Bode, W., 1978; Walsh, K. A., 1970; Read, R. J. et al., 1984; Fiedler, F. 1987; Fletcher, T. S. et al., 1987; Polgár, L. Structure and function of serine proteases. In New Comprehensive Biochemistry Vol. 16, Hydrolytic Enzymes (Neuberger, A. and Brocklehurst, K. eds), pp. 159-200, 1987; Tani, T., et al. 1990), and may be used in conjunction with the disclosures herein.

Examples of a trypsin and/or a functional equivalent KEEG sequences for production of wild-type and/or a functional equivalent nucleotide and protein sequence include: HSA-5644(PRSS1), 5645(PRSS2), 5646(PRSS3); PTR-747006(PRSS3); MCC-698352(PRSS2), 698729(PRSS1), 699238(PRSS2); MMU-22072(Prss2), 435889(1810049H19R1k), 436522(Try10); RNO-24691(Prss1), 25052(Prss2), 286960, 362347; CFA-475521(PRSS3); BTA-282603(PRSS2), 780933; MDO-100010059, 100010109, 100010619, 100010951; GGA-396344(PRSS2), 396345(PRSS3), 768632, 768663; XLA-379460(MGC64344); XTR-496623, 496627, 548509; DRE-65223(try); DME-Dmel_CG10232, Dmel_CG10405, Dmel_CG10586, Dmel_CG10587, Dmel_CG10663, Dmel_CG10764, Dmel_CG1102(MP1), Dmel_CG11037, Dmel_CG11192, Dmel_CG11313, Dmel_CG11668, Dmel_CG11670, Dmel_CG11836, Dmel_CG11841, Dmel_CG11842, Dmel_CG11843, Dmel_CG12350(lambdaTry), Dmel_CG12351(deltaTry);, Dmel_CG12385(thetaTry), Dmel_CG12386(etaTry); Dmel_CG12387(zetaTry), Dmel_CG1299, Dmel_CG13430, Dmel_CG13744; Dmel_CG14642, Dmel_CG14760, Dmel_CG16705(SPE), Dmel_CG16710, Dmel_CG16998, Dmel_CG17239, Dmel_CG17571, Dmel_CG1773, Dmel_CG18211(betaTry), Dmel_CG18444(alphaTry); Dmel_CG18681(epsilonTry), Dmel_CG18735, Dmel_CG18754, Dmel_CG2045(Ser7), Dmel_CG2056(spirit), Dmel_CG30002, Dmel_CG30025, Dmel_CG30031, Dmel_CG30371, Dmel_CG30414, Dmel_CG3066(Sp7); , Dmel_CG31219, Dmel_CG31265, Dmel_CG31269, Dmel_CG31681, Dmel_{—}0G31728, Dmel_CG31822, Dmel_CG31824, Dmel_CG31954, Dmel_CG32269, Dmel_CG32271, Dmel_CG32277, Dmel_CG32374, Dmel_CG32383(sphinx1), Dmel_CG32755, Dmel_CG32808, Dmel_{—}0G33127, Dmel_CG33276, Dmel_CG33461, Dmel_CG33462, Dmel_CG3355, Dmel_CG34350, Dmel_CG34409, Dmel_CG3650, Dmel_CG3700, Dmel_CG4053, Dmel_CG4316(Sb), Dmel_CG4386, Dmel_{—}0G4613, Dmel_CG4812(Ser8); , Dmel_CG4914, Dmel_CG4927, Dmel_CG5255, Dmel_CG5896(grass); Dmel_CG6041, Dmel_CG6048, Dmel_CG6361, Dmel_CG6367(psh); Dmel_CG6865, Dmel_{—}0G7432, Dmel_CG7754(iotaTry), Dmel_CG7829, Dmel_CG8170, Dmel_CG8172, Dmel_CG8213, Dmel_CG8299, Dmel_CG8870, Dmel_CG9294, Dmel_CG9372, Dmel_CG9564(Try29F), Dmel_CG9733, Dmel_CG9737; DPO-Dpse_GA11574, Dpse_GA11597, Dpse_GA11598, Dpse_GA11599, Dpse_GA14937, Dpse_GA15051, Dpse_GA15202, Dpse_GA15903, Dpse_GA18102, Dpse_GA19543, Dpse_GA20562, Dpse_GA21879; ANI-AN2366.2; BBA-Bd0564, Bd2630; MXA-MXAN_{—}5435; and/or SMA-SAV_{—}2443.

Structural information for a wild-type trypsin and/or a functional equivalent amino acid sequence for producing a trypsin and/or a functional equivalent include Protein database bank entries: 1A0J, 1AKS, 1AMH, 1AN1, 1ANB, 1ANC, 1AND, 1ANE, 1AQ7, 1AUJ, 1AVW, 1AVX, 1AZ8, 1BJU, 1BJV, 1BRA, 1BRB, 1BRC, 1BTP, 1BTW, 1BTX, 1BTY, 1BTZ, 1BZX, 1C1N, 1C1O, 1C1P, 1C1Q, 1C1R, 1C1S, 1C1T, 1C2D, 1C2E, 1C2F, 1C2G, 1C2H, 1C2I, 1C2J, 1C2K, 1C2L, 1C2M, 1C5P, 1C5Q, 1C5R, 1C5S, 1C5T, 1C5U, 1C5V, 1C9P, 1C9T, 1CE5, 1CO7, 1D6R, 1DPO, 1EB2, 1EJA, 1EJM, 1EPT, 1EZS, 1EZU, 1EZX, 1F0T, 1F0U, 1F2S, 1F5R, 1F7Z, 1FMG, 1FN6, 1FN8, 1FN1, 1FY4, 1FY5, 1FY8, 1G36, 1G3B, 1G3C, 1G3D, 1G3E, 1G9I, 1GBT, 1GDN, 1GDQ, 1GDU, 1GHZ, 1GI0, 1GI1, 1GI2, 1GI3, 1GI4, 1GI5, 1GI6, 1GJ6, 1H4W, 1H9H, 1H91, 1HJ8, 1HJ9, 1J14, 1J15, 1J16, 1J17, 1J8A, 1JIR, 1JRS, 1JRT, 1K1I, 1K1J, 1K1L, 1K1M, 1K1N, 1K1O, 1K1P, 1K9O, 1LDT, 1LQE, 1MAX, 1MAY, 1 MBQ, 1MCT, 1MTS, 1MTU, 1MTV, 1MTW, 1N6X, 1N6Y, 1NC6, 1NTP, 1O2H, 1O2I, 1O2J, 1O2K, 1O2L, 1O2M, 1O2N, 1O2O, 1O2P, 1O2Q, 1O2R, 1O2S, 1O2T, 1O2U, 1O2V, 1O2W, 1O2X, 1O2Y, 1O2Z, 1O30, 1O31, 1O32, 1O33, 1O34, 1O35, 1O36, 1037, 1038, 1O39, 1O3A, 1O3B, 1O3C, 1O3D, 1O3E, 1O3F, 1O3G, 1O3H, 1O3I, 1O3J, 1O3K, 1O3L, 1O3M, 1O3N, 1O3O, 1OPH, 1OS8, 1O SS, 1OX1, 1OYQ, 1P2I, 1P2J, 1P2K, 1PPC, 1PPE, 1PPH, 1PPZ, 1PQ5, 1PQ7, 1PQ8, 1PQA, 1QA0, 1QB1, 1QB6, 1QB9, 1QBN, 1QB0, 1QL7, 1QL8, 1QL9, 1QQU, 1RXP, 1S0Q, 1S0R, 1S5S, 1S6F, 1S6H, 1S81, 1S82, 1S83, 1S84, 1S85, 1SBW, 1SFI, 1SGT, 1SLU, 1SLV, 1SLW, 1SLX, 1SMF, 1TAB, 1TAW, 1TFX, 1T10, 1TLD, 1TNG, 1TNH, 1TNI, 1TNJ, 1TNK, 1TNL, 1TPA, 1TPO, 1TPP, 1TRM, 1TRN, 1TRY, 1TX7, 1TX8, 1UHB, 1UTJ, 1UTK, 1UTL, 1UTM, 1UTN, 1UTO, 1UTP, 1UTQ, 1V2J, 1V2K, 1V2L, 1V2M, 1V2N, 1V2O, 1V2P, 1V2Q, 1V2R, 1V2S, 1V2T, 1V2U, 1V2V, 1V2W, 1V6D, 1XUF, 1XUG, 1XUH, 1XUI, 1XUJ, 1XUK, 1XVM, 1XVO, 1Y3U, 1Y3V, 1Y3W, 1Y3X, 1Y3Y, 1Y59, 1Y5A, 1Y5B, 1Y5U, 1YF4, 1YKT, 1YLC, 1YLD, 1YP9, 1YYY, 1Z7K, 1ZR0, 2A31, 2A32, 2A7H, 2AGE, 2AGG, 2AGI, 2AH4, 2AYW, 2BLV, 2BLW, 2BTC, 2BY5, 2BY6, 2BY7, 2BY8, 2BY9, 2BYA, 2BZA, 2CMY, 2D8W, 2EEK, 2F3C, 2F91, 2F13, 2F14, 2F15, 2FMJ, 2FTL, 2FTM, 2FX4, 2FX6, 2G51, 2G52, 2G55, 2G5N, 2G5V, 2G8T, 21LN, 2J9N, 2O9Q, 2OTV, 2OXS, 2PLX, 2PTC, 2PTN, 2QN5, 2R9P, 2RA3, 2STA, 2STB, 2TBS, 2T10, 2TLD, 2TRM, 2UUY, 2VU8, 2ZDK, 2ZDL, 2ZDM, 2ZDN, 2ZFS, 2ZFT, 3BEU, 3BTD, 3BTE, 3BTF, 3BTG, 3BTH, 3BTK, 3BTM, 3BTQ, 3BTT, 3BTW, 3PTB, 3PTN, 3TGI, 3TGJ, 3TGK, and/or 5PTP.

b. Chymotrysins

Chymotrypsin (EC 3.4.21.1) has been also referred to as “chymotrypsins A and B,” “α-chymar ophth,” “avazyme,” “chymar,” “chymotest,” “enzeon,” “quimar,” “quimotrase,” “α-chymar,” “α-chymotrypsin A,” and/or “α-chymotrypsin.” A chymotrypsin generally cleaves peptide bonds at the carboxyl side of amino acids, with a preference for a substrate comprising a Tyr, a Trp, a Phe, and/or a Leu. As with other peptidases, chymotrypsin producing cells and methods for isolating a chymotrypsin from a cellular material and/or a biological source have been described, [see, for example, Dodgson, K. S. et al., 1956; Roy, A. B. 1960; Roy, A. B., 1976; Webb, E. C. and Morrow, P. F. W., 1959), and may be used in conjunction with the disclosures herein.

Examples of a chymotrypsin and/or a functional equivalent KEEG sequences for production of wild-type and/or a functional equivalent nucleotide and protein sequence include: HSA-1504(CTRB1), 440387(CTRB2); PTR-736467(CTRB1); MCC-711100, 713851(CTRB1); MMU-66473(Ctrb1); RNO-24291(Ctrb1); CFA-479649(CTRB2), 479650(CTRB1), 610373; BTA-504241(CTRB1); XLA-379495, 379607(MGC64417), 444360; XTR-496968(ctrl), 548358(ctrb1); DRE-322451(ctrb1), 562139; NVE-NEMVE_v1g140545; DME-Dmel_CG10472, Dmel_CG11529, Dmel_CG11911, Dmel_CG16996, Dmel_CG16997, Dmel_CG17234, Dmel_CG17477, Dmel_CG18179, Dmel_CG18180, Dmel_CG31362(Jon99Ciii), Dmel_CG3916, Dmel_CG6298(Jon74E), Dmel_CG6457(yip7), Dmel_CG6467(Jon65Aiv), Dmel_CG6592, Dmel_CG7142, Dmel_CG7170(Jon66Cii), Dmel_CG7542, Dmel_CG8329, Dmel_CG8579(Jon44E), Dmel_CG8869(Jon25Bii); DPO-Dpse_GA19618, and/or Dpse_GA21380.

Structural information for a wild-type chymotrypsin and/or a functional equivalent amino acid sequence for producing a chymotrypsin and/or a functional equivalent include Protein database bank entries: 1AB9, 1ACB, 1AFQ, 1CA0, 1CBW, 1CHO, 1DLK, 1EQ9, 1EX3, 1GCD, 1GCT, 1GG6, 1GGD, 1GHA, 1GHB, 1GL0, 1GL1, 1GMC, 1GMD, 1GMH, 1HJA, 1K2I, 1kDQ, 1MTN, 1N8O, 1OXG, 1P2M, 1P2N, 1P2O, 1P2Q, 1T7C, 1T8L, 1T8M, 1T8N, 1T8O, 1VGC, 1YPH, 2CHA, 2GCH, 2GCT, 2GMT, 2JET, 2P8O, 2VGC, 3BG4, 3GCH, 3GCT, 3VGC, 4CHA, 4GCH, 4VGC, 5CHA, 5GCH, 6CHA, 6GCH, 7GCH, and/or 8GCH.

c. Chymotrypsins C

Chymotrypsin C (EC 3.4.21.2; CAS no. 9036-09-3) hydrolyzes a peptide bond, particularly those comprising a Leu, a Tyr, a Phe, a Met, a Trp, a Gln, and/or an Asn. Chymotrypsin C producing cells and methods for isolating a chymotrypsin C from a cellular material and/or a biological source have been described, [see, for example, Peanasky, R. J. et al., 1969; Folk, J. E., 1970; and Wilcox, P. E., 1970], and may be used in conjunction with the disclosures herein. Structural information for a wild-type chymotrypsin C and/or a functional equivalent amino acid sequence for producing a chymotrypsin C and/or a functional equivalent include Protein database bank entries: HSA*-*11330(CTRC); PTR*-*739685(CTRC); MCC*-*700270, 700762(CTRC); MMU*-*76701(Ctrc); RNO*-*362653(Ctrc); CFA**478220(CTRC); and/or BTA*-*514047(CTRC).

d. Subtilisins

Subtilisin (EC 3.4.21.62; CAS No. 9014-01-1) has been also referred to as “alcalase 0.6 L,” “alcalase 2.5 L,” “alcalase,” “alcalase,” “ALK-enzyme,” “bacillopeptidase A,” “bacillopeptidase B,” “Bacillus subtilis alkaline proteinase bioprase,” “Bacillus subtilis alkaline proteinase,” “bioprase AL 15,” “bioprase APL 30,” “colistinase,” “esperase,” “genenase I,” “kazusase,” “maxatase,” “opticlean,” “orientase 10B,” “protease S,” “protease VIII,” “protease XXVII,” “protin A 3 L,” “savinase 16.0 L,” “savinase 32.0 L EX,” “savinase 4.0 T,” “savinase 8.0 L,” “savinase,” “SP 266,” “subtilisin BL,” “subtilisin DY,” “subtilisin E,” “subtilisin GX,” “subtilisin J,” “subtilisin S41,” “subtilisin Sendai,” “subtilopeptidase,” “superase,” “thermoase PC 10,” or “thermoase.” A subtilisin comprises a serine endopeptidase, and hydrolyzes a peptide bond, particularly those comprising a bulky uncharged P1 residue; as well as hydrolyzes a peptide amide bond. Subtilisin producing cells and methods for isolating a subtilisin from a cellular material and/or a biological source have been described, [see, for example, Nedkov, P., et al., 1985; Ikemura, H., et al., 1987), and may be used in conjunction with the disclosures herein. In some aspects, a subtilisin has esterase activity.

Examples of a subtilisin and/or a functional equivalent KEEG sequences for production of wild-type and/or a functional equivalent nucleotide and protein sequence include: DME-Dmel_CG7169(S1P); OSA-433-4194(Os03g0761500); ANG-An09g03780(pepD); PFA-PFE0370c; PEN-PSEEN4433; CPS-CPS_{—}0751; AZO-azo1237(subC); GSU-GSU2075; GME-Gmet_{—}0931; RLE-RL1858; BRA-BRADO0807; RDE-RD1_{—}4002(apr); BSU-BSU10300(aprE); BHA-BH0684(alp) BH0855; BTL-BALH_{—}4378; BLI-BL01111(apr); BLD-BLi01109; BCL-ABC0761(aprE); DRM-Dred_{—}0089; MTA-Moth_{—}2027; MPU-MYPU_{—}6550; MHJ-MHJ_{—}0085; RHA-RHA1_ro08410; SEN-SACE_{—}7133(aprE); RBA-RB841; AVA-Ava_{—}2018 and/or Ava_{—}4060.

Structural information for a wild-type subtilisin and/or a functional equivalent amino acid sequence for producing a subtilisin and/or a functional equivalent include Protein database bank entries: 1A2Q, 1AF4, 1AK9, 1AQN, 1AU9, 1AV7, 1AVT, 1BE6, 1BE8, 1BFK, 1BFU, 1BH6, 1C3L, 1C9J, 1C9M, 1C9N, 1CSE, 1DUI, 1GCI, 1GNS, 1GNV, 1IAV, 1JEA, 1LW6, 1 MPT, 1NDQ, 1NDU, 1OYV, 1Q5P, 1R0R, 1SBC, ISBN, 1SBI, 1SBN, 1SCA, 1SCB, 1SCD, 1SCJ, 1SCN, 1SIB, 1SPB, 1ST3, 1SUA, 1SUB, 1SUC, 1SUD, 1SUE, 1SUP, 1SVN, 1TK2, 1TM1, 1TM3, 1TM4, 1TM5, 1TM7, 1TMG, 1TO1, 1TO2, 1UBN, 1V5I, 1VSB, 1Y1K, 1Y33, 1Y34, 1Y3B, 1Y3C, 1Y3D, 1Y3F, 1Y48, 1Y4A, 1Y4D, 1YU6, 2E1P, 2GKO, 2SEC, 2Z2X, 2Z2Y, 2Z2Z, 2Z30, 2Z56, 2Z57, 2Z58, 3BGO, 3BX1, 3CNQ, 3CO0, 3F49, 3SIC, 3VSB, and/or 5SIC.

5. Peroxidases

A typically peroxidase (EC 1.11.1) catalyzes a reaction of hydrogen peroxide on a substrate (“donor”) to add an oxygen moiety via the reaction: donor+H₂O₂=oxidized donor+2H₂O. A peroxidase may be categorized by the donor. Examples of a peroxidase includes a NADH peroxidase (EC 1.11.1.1; CAS registry number: 9032-24-0), which uses a NADH as a donor; a NADPH peroxidase (EC 1.11.1.2; CAS registry number: 9029-51-0), which uses a NADPH as a donor; a fatty-acid peroxidase (EC 1.11.1.3; CAS registry number: 9029-52-1), which uses a palmitate as a donor; a cytochrome-c peroxidase (EC 1.11.1.5; CAS registry number: 9029-53-2), which uses a ferrocytochrome c as a donor; a catalase (EC 1.11.1.6; CAS registry number: 9001-05-2), which uses a H₂O₂ as a donor; a peroxidase (EC 1.11.1.7; CAS registry number: 9003-99-0), which uses various substrates as a donor; an iodide peroxidase (EC 1.11.1.8; CAS registry number: 9031-28-1), which uses an iodide as a donor; a glutathione peroxidase (EC 1.11.1.9; CAS registry number: 9013-66-5), which uses a glutathione as a donor; a chloride peroxidase (EC 1.11.1.10; CAS registry number: 9055-20-3); a L-ascorbate peroxidase (EC 1.11.1.11; CAS registry number: 72906-87-7), which uses a L-ascorbate as a donor; a phospholipid-hydroperoxide glutathione peroxidase (EC 1.11.1.12; CAS registry number: 97089-70-8), which uses a glutathione and a lipid hydroperoxide as a donor; a manganese peroxidase (EC 1.11.1.13; CAS registry number: 114995-15-2), which uses a Mn(II) and a H⁺ as a donor; a lignin peroxidase (EC 1.11.1.14; CAS registry number: 93792-13-3), which uses a 1,2-bis(3,4-dimethoxyphenyl)propane-1,3-diol as a donor; a peroxiredoxin (EC 1.11.1.15; CAS registry number: 207137-51-7); a versatile peroxidase (EC 1.11.1.16; CAS registry number: 42613-30-9, 114995-15-2); or a combination thereof.

a. Peroxidases (EC 1.11.1.7)

Peroxidase (EC 1.11.1.7; CAS registry number: 9003-99-0) has been also referred to as “myeloperoxidase,” “lactoperoxidase,” “verdoperoxidase,” “guaiacol peroxidase,” “thiocyanate peroxidase,” “eosinophil peroxidase,” “Japanese radish peroxidase,” “horseradish peroxidase (HRP),” “extensin peroxidase,” “heme peroxidase,” “MPO,” “oxyperoxidase,” “protoheme peroxidase,” “pyrocatechol peroxidase,” “scopoletin peroxidase,” and/or “donor:hydrogen-peroxide oxidoreductase.” A peroxidase (EC 1.11.1.7) may be referred herein by its EC classification number (EC 1.11.1.7) to distinguish from the subgenus of “peroxidases,” which are referred to herein by the EC classification number (EC 1.11.1). A peroxidase (EC 1.11.1.7) catalyzes a reaction of hydrogen peroxide on a substrate (“donor”) to add an oxygen moiety via the reaction: donor+H2O2=oxidized donor+2H2O. A peroxidase generally comprises a hemoprotein. Peroxidase (EC 1.11.1.7) producing cells and methods for isolating a peroxidase from a cellular material and/or a biological source have been described [see, for example, Kenten, R. H. and Mann, P. J. G., 1954; Morrison, M. et al., 1957; Paul, K. G. Peroxidases. In: Boyer, P. D., Lardy, H. and Myrbäck, K. (Eds.), The Enzymes, 2nd ed., vol. 8, Academic Press, New York, p. 227-274, 1963; Tagawa, K. et al., 1959; Theorell, H., 1943], and may be used in conjunction with the disclosures herein.

Examples of a peroxidase (EC 1.11.1.7) and/or a functional equivalent KEEG sequences for production of wild-type and/or a functional equivalent nucleotide and protein sequence include: HSA-4025(LPO), 4353(MPO), 8288(EPX), 9588(PRDX6); PTR-468-420(EPX), 469589(PRDX6), 738041(PRDX6), 748680(MPO); MCC-706486(PRDX6), 707299, 709655(EPX), 709848(LPO), 714246(MPO); MMU-11758(Prdx6), 13861(Epx), 17523(Mpo), 320769(Prdx6-rs1), 76113(Lpo); RNO-303413(Mpo), 303414(Epx), 94167(Prdx6); CFA-480069(PRDX6), 491109(EPX), 491111(LPO), 609986(MPO); BTA-280844(LPO), 282438(PRDX6), 786533; SSC-399538(PRDX6); MDO-100012462, 100015705; GGA-417-467(MPO), 429062(PRDX6); XLA-394386(mpo-A), 398641, 399434(prdx6); XTR-394706, 496787(prdx6); DRE-393778(prdx6); SPU-579284; NVE-NEMVE_v1g234225; DME-Dmel_CG10211, Dmel_CG10793, Dmel_CG11765(Prx2540-2); Dmel_CG12002(Pxn), Dmel_CG12199(kek5), Dmel_CG12896, Dmel_CG13889; Dmel_CG1804(kek6), Dmel_CG2019(disp), Dmel_CG3131(Duox); Dmel_CG3477(Pxd), Dmel_CG4009, Dmel_CG4977(kek2), Dmel_CG5873; Dmel_CG6879, Dmel_CG6969, Dmel_CG7660(pxt), Dmel_CG8913(Irc); DPO-Dpse_GA10160, Dpse_GA16169, Dpse_GA21405; CEL-F09F3.5, T06D8.10(peroxidase); ATH-AT1G05260(RCI3), AT1G14540, AT1G14550, AT1G24110, AT1G30870; AT1G34510, AT1G44970, AT1G49570, AT1G68850, AT1G71695, AT2G18140; AT2G18150, AT2G18980, AT2G22420, AT2G24800, AT2G34060, AT2G37130; AT2G38380, AT2G38390, AT2G39040, AT2G41480, AT2G43480, AT3G01190; AT3G03670, AT3G17070, AT3G21770, AT3G28200; AT3G49110(ATPCA/ATPRX33/PRX33/PRXCA); AT3G49120(ATPCB/ATPERX34/PERX34/PRXCB), AT3G49960, AT4G08770; AT4G08780, AT4G11290, AT4G16270, AT4G17690, AT4G21960(PRXR1); AT4G26010, AT4G30170, AT4G31760, AT4G33420, AT4G36430, AT4G37520; AT4G37530, AT5G05340, AT5G06720, AT5G06730, AT5G14130, AT5G15180; AT5G17820, AT5G19880, AT5G19890, AT5G22410, AT5G24070, AT5G40150; AT5G42180, AT5G47000, AT5G51890, AT5G58390, AT5G58400, AT5G64100; AT5G64110, AT5G64120, AT5G66390, AT5G67400; OSA-432-4557(Os01g0963200), 4325127(Os01g0263000); 4326874(Os01g0543100), 4330684(Os02g0741200); 4332174(Os03g0234900), 4332175(Os03g0235000); 4335846(Os04g0423800), 4338164(Os05g0231900); 4340745(Os06g0274800), 4341861(Os06g0681600); 4342251(Os07g0115300), 4343309(Os07g0499500); 434-4496(Os08g0113000), 4345222(Os08g0302000); 4347336(Os09g0471100), 4349587(Os11g0112400); CME-CMCO₃₉C; DHA-DEHA0F10593g; NCR-NCU06031; AFM-AFUA_{—}4G08580; DDI-DDB_{—}0238006; PFA —PFL0595c; PPU-PP_{—}0235(IsfA); PFL-PFL_{—}5939; PEN-PSEEN0215(IsfA); PSA-PST_{—}0214(IsfA); PRW-PsycPRwf_{—}1436; ACB-A1S_{—}2863; ABY-ABAYE0619; MAQ-Maqu_{—}0254; NOC-Noc_{—}0878, Noc_{—}1307; CSA-Csal_{—}0179; CVI-CV 1938, CV 3739; RSO-RSc0754(RS05099); REU-Reut_B4984; REH-H16_A2819; RME-Rmet_{—}2654, Rmet_{—}4131; BMA-BMA2066; BMV-BMASAVP1_A0844; BML-BMA10229_A2677; BMN-BMA10247_{—}1932; BXE-Bxe_B2802; BVI-Bcep1808_{—}0748; BUR-Bcep18194_A3905, Bcep18194_B0181, Bcep18194_B1953; BCN-Bcen_{—}0329; BCH-Bcen2424_{—}0812; BAM-Bamb_{—}0693; BPS-BPSL2748; BPM-BURPS1710b_{—}3239(IsfA); BPL-BURPSI106A_{—}3223(IsfA); BPD-BURPS668_{—}3186(IsfA); BTE-BTH_I1388; POL-Bpro_{—}2374, Bpro_{—}4841; VEI-Veis_{—}0864; HAR-HEAR3137; AZO-azo2663; DVU-DVU2247; RET-RHE_CH01791(ypch00605); RLE-RL2003; RPA-RPA2443; RPB-RPB_{—}3015; NWI-Nwi_{—}1738; NHA-Nham_{—}2167; JAN-Jann_{—}4026; RDE-RD1_{—}0634; PDE-Pden_{—}2756; MMR -Mmar10_{—}0498; GBE-GbCGDNIH1_{—}0908; ACR-Acry_{—}2948; SUS-Acid_{—}5901; FAL-FRAAL0302, FRAAL4492(ahpC); RBA-RB11131, RB4293, RB633; TER-Tery_{—}5038; FJO-Fjoh_{—}5017; and/or NPH-NP2708A(perA)

Structural information for a wild-type peroxidase (EC 1.11.1.7) and/or a functional equivalent amino acid sequence for producing a peroxidase and/or a functional equivalent include Protein database bank entries: 1ARP; 1ARU; 1ARV; 1ARW; 1ARX; 1ARY; 1ATJ; 1BGP; 1C8I; 1CK6; 1CXP; 1D2V; 1D5L; 1D7W; 1DNU; 1DNR; 1FHF; 1GW2; 1GWO; 1GWT; 1GWU; 1GX2; 1GZA; 1GZB; 1H3J; 1H55; 1H57; 1H58; 1H5A; 1H5C; 1H5D; 1H5E; 1H5F; 1H5G; 1H5H; 1H5I; 1H5J; 1H5K; 1H5L; 1H5M; 1NCH; 1HSR; 1KZM; 1LY8; 1LY9; 1LYC; 1LYK; 1 MHL; 1MNP; 1MYP; 1PA2; 1QO4; 1SCH; 1W4W; 1W4Y; 1XXU; 2ATJ; 2C0D; 2E39; 2E3A; 2E3B; 2E9E; 2EFB; 2EHA; 2GJ1; 2GJM; 21KC; 21PS; 2NQX; 2O86; 2OJV; 2PT3; 2PUM; 2QPK; 2QQT; 2QRB; 2R5L; 2Z5Z; 3ATJ; 3BXI; 4ATJ; 6ATJ; and/or 7ATJ.

### C. ANTIBIOLOGICAL AGENTS INCLUDING PEPTIDES, POLYPEPTIDES, AND ENZYMES

In many embodiments, a material formulation (e.g., a surface treatment, a filler, a biomolecular composition, a textile finish, etc.) comprises an antibiological agent. An antibiological agent may comprise a biomolecular composition such as a proteinaceous molecule (“antibiological proteinaceous molecule”) such as an enzyme, a peptide, a polypeptide, or a combination thereof. A material formulation may comprise an antibiological agent by being formulated, prepared, processed, post-cured processed, manufactured, and/or applied (e.g., applied to a surface), in a fashion to be suitable to possess an antibiological activity and/or function (e.g., an antimicrobial activity, an antifouling activity). In specific aspects, antibiological agent (e.g., an antimicrobial agent, an antifouling agent) may act against a biological entity (e.g., a cell, a virus) that contacts (e.g., a surface contact, an internal incorporation, an infiltration, an infestation) a material formulation.

An antibiological agent may act by treating an infestation, preventing infestation, inhibiting infestation (e.g., preventing cell attachment), inhibiting growth, preventing growth, lysing, and/or killing; a biological entity such as a cell and/or a virus (e.g., one or more genera and/or species of a cell and/or a virus). Thus, some embodiments comprise a process for treating an infestation, preventing infestation, inhibiting infestation (e.g., preventing cell attachment), inhibiting growth, preventing growth, lysing, and/or killing a cell and/or a virus (e.g., a fungal cell) comprising contacting the cell and/or the virus with a material formulation (e.g., a paint, a coating composition, a biomolecular composition) comprising at least one proteinaceous molecule (e.g., an effective amount of an antibiological peptide, antibiological polypeptide, an antibiological enzyme, and/or an antibiological protein). In some aspects, such an antibiological agent (e.g., an antibiological proteinaceous molecule) may possess a biocidal and/or a biostatic activity. For example, an antimicrobial and/or an antifouling enzyme may act as a biocide and/or a biostatic. In some embodiments, an antibiological proteinaceous molecule (e.g., a biostatic) may inhibit growth of a cell and/or a virus, which refers to cessation and/or reduction of cell (e.g., a fungal cell) and/or viral proliferation, and can also include inhibition of expression of cellullarly produced proteins in a static cell colony. For example, a coating comprising an antimicrobial agent may act against a microbial cell and/or a virus adapted for growth in a non-marine environment and/or does not produces fouling; while a coating comprising an antifouling agent may act against a marine cell that produces fouling. In another example, a virus may be a target of such an antibiological agent, as the virus (e.g., a membrane enveloped virus) may comprise a biomolecule target of an antibiological agent (e.g., an enzyme, an antibiological proteinaceous molecule such as a peptide).

In some embodiments, a target cell and/or a target virus may be capable of infesting an inanimate object (e.g., a building material, an indoor structure, an outdoor structure). An “inanimate object” refers to structures and objects other than a living cell (e.g., a living organism). Examples of an inanimate object include an architectural structure that may comprise a painted and/or an unpainted surface such as the exterior wall of a building; the interior wall of a building; an industrial equipment; an outdoor sculpture; an outdoor furniture; a construction material for indoor and/or outdoor use such as a wood, a stone, a brick, a wall board (e.g., a sheetrock), a ceiling tile, a concrete, an unglazed tile, a stucco, a grout, a roofing tile, a shingle, a painted and/or a treated wood, a synthetic composite material, a leather, a textile, or a combination thereof. Such an inanimate object may comprise (e.g., a plastic building material, a wood coated with a surface treatment) a material formulation. Examples of a building material includes a conventional and/or a non-conventional indoor and/or an outdoor construction and/or a decorative material, such as a wood; a sheet-rock (e.g., a wallboard); a paper and/or vinyl coated wallboard; a fabric (e.g., a textile); a carpet; a leather; a ceiling tile; a cellulose resin wall board (e.g., a fiberboard); a stone; a brick; a concrete; an unglazed tile; a stucco; a grout; a painted surface; a roofing tile; a shingle; a cellulose-rich material; a material capable of providing nutrient(s) to a cell (e.g., fungi) and/or a virus, capable of harboring nutrient material(s) and/or supporting a biological (e.g., a fungal) infestation; or a combination thereof.

One or more cells (e.g., a fungus) and/or viruses may, for example, infest, survive upon, survive within, grow on the surface, and/or grow within, an inanimate object. Such a target cell and/or a target virus (e.g., a fungal cell) include those that can infest and/or survive upon and/or within: an inanimate object such as an indoor structure, an outdoor structure, a building material, or a combination thereof, and may cause defacement (e.g., deterioration or discoloration), odor, environment hazards, and other undesirable effects.

A material (e.g., an object) may be susceptible (“prone”) to infestation by a cell and/or a virus when it is capable of serving as a food source for a cell (e.g., the material comprises a substance that serves as a food source). It is contemplated that any described formulation of a cell and/or a virus (e.g., a fungus) prone material formulation may be modified to incorporate an antibiological agent (e.g., an antifungal peptidic agent). For example, in the context of a paint or coating composition, a fungal-prone material may comprise a binder comprising a carbon-based polymer that serves as a nutrient for a fungus, and a coating comprising the binder as a component may also comprise an antibiological proteinaceous composition. In another example, a susceptible material formulation such as a grout and/or a caulk that may be in frequent contact with or constantly exposed to fungal nutrients and moisture may comprise a proteinaceous molecule effective against a fungus on and/or within the susceptible material formulation (e.g., a surface).

Antibiological activity (e.g., growth inhibition, biocidal activity) can provide and/or facilitate disinfection, decontamination and/or sanitization of an material and/or an object (e.g., an inanimate object, a building material), which refer to the process of reducing the number of cell(s) (e.g., a fungus microorganism) and/or viruses to levels that no longer pose a threat (e.g., a threat to property, a threat to the health of a desired organism such as human). Use of a bioactive antifungal agent can be accompanied by removal (e.g., manual removal, machine aided removal) of the cell(s) and/or the virus(s).

In another example, a material formulation (e.g., a surface treatment) comprising an antimicrobial proteinaceous composition may be used in an application such as a hospital and/or a health care application, such as reducing and/or preventing a hospital-acquired infection (e.g., a so-called “super bugs” infection); and/or reducing (e.g., reducing the spread) and/or preventing infection(s) (e.g., a viral infection such as SARS); as well as a hygienic surface application (e.g., an antimicrobial cleaner, an antimicrobial utensil, an antimicrobial food preparation surface, an antimicrobial coating system); reducing and/or preventing food poisoning; or a combination thereof. Examples of a strain of bacteria that may be resistant to a conventional antibiotic, such as a Staphalococcus [e.g., a Methicillin-resistant Staphylococcus aureus (“MRSA”)], a Streptococcus bacteria, and/or a Vero-cytotoxin producing variants of Escherichia coli.

Methods for assaying and/or selecting an antibiotic composition are described in U.S. Pat. Nos. 6,020,312; 5,885,782; and 5,602,097, and patent application Ser. Nos. 10/884,355 and 11/368,086, such as, for example, contacting a material formulation (e.g., a coating) comprising a proteinaceous molecule (e.g., a peptide) with a biological cell (e.g., a fungal cell) and/or a virus, and measuring growth over time relative to a like material formulation comprising less or no selected proteinaceous molecule content. For example, a fungal cell may be used in assaying and/or screening for an antifungal composition (e.g., a peptide library), may comprise a fungal organism known to, or suspected of, infesting a vulnerable material(s) and/or surface(s) (e.g., a construction material). Such methods may be used to assay and/or screen, for example, antifungal activity against a wide variety of fungus genera and species, such as in the case of selecting a composition comprising a broad-spectrum antifungal activity. Similar methods may be used to identify particular proteinaceous composition(s) (e.g., a peptide, a plurality peptides) that target specific fungus genera or species. Examples of such a fungal cell often used in such an assay include members of the genera Stachybotrys (especially Stachybotrys chartarum), Aspergillus species (sp.), Penicillium sp., Fusarium sp., Alternaria dianthicola, Aureobasidium pullulans (aka Pullularia pullulans), Phoma pigmentivora and Cladosporium sp, though an assay may be adapted for other cell(s). In another example, a proteinaceous molecule (e.g., a peptide) may be effective (e.g., inhibit growth, treat infestation, etc.) against a cell (e.g., a fungal cell, a bacterial cell) and/or a virus from a genera and/or a species of, for example, an Alternaria (e.g., an Alternaria dianthicola), an Aspergillus [(e.g., an Aspergillus species (sp.), an Aspergillus fumigatus, an Aspergillus Parasiticus], an Aureobasidium (e.g., an Aureobasidium pullulans a.k.a. a Pullularia pullulans), a Candida; a Ceratocystis (e.g., a Ceratocystis Fagacearum), a Cladosporium (e.g., a Cladosporium sp.), a Fusarium (e.g., a Fusarium sp., a Fusarium oxysporum, a Fusariam Sambucinum), a Magaporthe (e.g., a Magaporthe Aspergillus nidulans), a Mycosphaerella, a Penicillium (e.g., a Penicillium sp.), a Phoma (e.g., a Phoma pigmentivora), a Pphiostoma (e.g., a Pphiostoma ulmi), a Pythium (e.g., a Pythium ultimum, a Rhizoctonia (e.g., Rhizoctonia Solani), a Stachybotrys (e.g., a Stachybotrys chartarum), or a combination thereof. Cell and/or viral culture conditions may be modified appropriately to provide favorable growth and proliferation conditions, using the techniques of the art, and to assay and/or screen for activity against a target cell (e.g., a bacteria, an algae, etc.) and/or a virus. Any suitable peptide/polypeptide/protein screening method in the art may be used to identify an antibiological proteinaceous molecule (e.g., an antifungal peptide) for an assay as active antibiological agent (e.g., an antifungal agent) in a material formulation (e.g., a paint, a coating material, a biomolecular composition). For example, an in vitro method to determine bioactivity of a peptide, such as a peptide from a synthetic peptide combinational library, may be used (Furka, A., et al., 1991; Houghten, R. A., et al., 1991; Houghten, R. A., et al., 1992).

An antibiological biomolecular composition may be combined with any other antibiological agent described herein and/or known in the art, such as a preservative (e.g., a chemical biocide, a chemical biostatic) typically used in a surface treatment (e.g., a coating, a paint) and/or an antimicrobial agent (e.g., a chemical biocide, a chemical biostatic) typically used in a polymeric material (e.g., a plastic, an elastomer, etc). For example, one or more antibiological proteinaceous molecule(s) (e.g., an antifungal peptidic agent, an enzyme) may be used in combination with and/or as a substitute for one or more existing antibiological agents (e.g., a preservative, an antimicrobial agent, a fungicide, a fungistatic, a bactericide, an algaecide, etc.) identified herein and/or in the art. Examples of an antibiological agent (e.g., a preservative) that an antibiological proteinaceous molecule (e.g., an antimicrobial proteinaceous molecule, an antifungal peptidic agent, an antimicrobial enzyme) may substitute for and/or be combined include, but are not limited to those non-peptidic antimicrobial compounds (I.e., biocides, fungicides, algaecides, mildewcides, etc.) which have been shown to be of utility and are currently available and approved for use in the U.S./NAFTA, Europe, and the Asia Pacific region, and numerous examples are described herein for use with a material formulation such as a surface treatment (e.g., a coating), etc. Some such combinations of antibiological proteinaceous molecule(s) and/or combinations with another antibiological agent may provide an advantage such as a broader range of activity against various organisms (e.g., a bacteria, an algae, a fungi, etc.), a synergistic antibiological and/or preservative effect, a longer duration of effect, or a combination thereof. For example, a fungal prone composition and/or a surface coated with such a composition are also susceptible to damage by a variety of organisms, and a combination of antibiological agents may protect against the variety of organisms. In another example of a combination, an antimicrobial and/or an antifouling agent comprising an enzyme (e.g., an antimicrobial enzyme, an antifouling enzyme) and/or a peptide (e.g., an antifouling peptide, an antimicrobial peptide, an antifungal peptide, an antialgae peptide, an antibacterial peptide, an antimildew peptide, etc) may be used alone or in combination with one or more additional antibiological agent(s) (e.g., an antimicrobial agent, an antifouling agent, a preservative, a biocide, a biostatic agent) and/or technique (see for example, Baldridge, G. D. et al, 2005; Hancock, R. E. W. and Scott, M. G., 2000).

In particular aspects, an antimicrobial peptide comprises ProteCoat® (Reactive Surfaces, Ltd.; also described in U.S. Pat. Nos. 6,020,312; 5,885,782; and 5,602,097, and patent application Ser. Nos. 10/884,355 and 11/368,086). For example, certain peptides contemplated for use (e.g., ProteCoat®; Reactive Surfaces, Ltd.) as described herein have been shown to involve synergy between the peptides (e.g., antifungal peptides) and non-peptide antifungal agents that may be useful in controlling growth of a Fusarium, a Rhizoctonia, a Ceratocystis, a Pythium, a Mycosphaerella, an Aspergillus and/or a Candida genera of fungi. In particular, synergistic combinations have been described and successfully used to inhibit the growth of an Aspergillus fumigatus and an A. paraciticus, and also an Fusarium oxysporum with respect to agricultural applications. These and other synergistic combinations of peptide and non-peptide agent(s) may be useful as, for example, a component (e.g., an additive) in a material formulation (e.g., a paint, a coating) such as for deterring, preventing, and/or treating a fungal infestation.

In some aspects, an antibiological agent (e.g., an antimicrobial agent, an antifouling agent) and/or technique comprises a detergent (e.g., a nonionic detergent, a zwitterionic detergent, an ionic detergent), such as CHAPS (zwitterionic), a Triton X series detergent (nonionic), and/or a SDS (ionic); a basic protein such as a protamine; a cationic polysaccharide such as chitosan; a metal ion chelator such as EDTA; or a combination thereof, all of which have may have effectiveness against a lipid cellular membrane, and may be incorporated into a material formulation and/or used in a washing composition (e.g., a washing solution, a washing suspension, a washing emulsion) applied to a material formulation. For example, a material formulation comprising an antimicrobial peptide and an antimicrobial enzyme may be washed with a commercial washing solution that may also comprise an antimicrobial peptide. In another example, an additional preservative, an biocide, an biostatic agent, or a combination thereof, comprises a non-peptidic antimicrobial agent, a non-amino based antimicrobial agent, a compounded peptide antimicrobial agent, an enzyme-based antimicrobial agent, or a combination thereof, such as those described in U.S. patent application Ser. No. 11/865,514 filed Oct. 1, 2007, incorporated by reference. In another example, an antibiological agent (e.g., an antimicrobial agent, an antifouling agent) may comprise components such as a Protecoat® combined with a non-peptidic antimicrobial agent, a non-amino based antimicrobial agent, a compounded peptide antimicrobial agent, an enzyme-based antimicrobial agent, or a combination thereof, and an improved (e.g., additive, synergistic) effect may occur, so that the concentration of one or more components of the antibiological agent may be reduced relative to the component's use alone or in a combination comprising fewer components. In some embodiments, the concentration of any individual antibiological agent component (e.g., an antimicrobial component, an antifouling component) comprises about 0.000000001% to about 20% (e.g., about 0.000000001% to about 4%) or more, of a material formulation, an antibiological agent (e.g., an antimicrobial agent, an antifouling agent), a washing composition, or a combination thereof.

Of course, an antibiological agent (e.g., an antimicrobial agent, an antifouling agent, an enzyme, a peptide, a preservative) may be combined with another biomolecular composition (e.g., an enzyme, a cell based particulate material), for the purpose to confer an additional property (e.g., a catalytic activity, a binding property) other than one related to antimicrobial and/or antifouling function. Examples of another biomolecular composition include an enzyme such as a lipolytic enzyme, though some lipolytic enzymes may have antimicrobial and/or antifouling activity; a phosphoric triester hydrolase; a sulfuric ester hydrolase; a peptidase, some of which may have an antimicrobial and/or antifouling activity; a peroxidase, or a combination thereof. Alternatively, in several embodiments, a biomolecular composition may be used with little or no antimicrobial and/or antifouling function. For example, a material formation may comprise a combination of active enzymes with little or no active antimarine, antifouling, and/or antimicrobial enzyme present.

1. Antibiolodical Enzymes

In many aspects, an antibiological agent comprises an enzyme (e.g., an antimicrobial enzyme, an antifungal enzyme, an antialgae enzyme, an antibacterial enzyme, antimildew enzyme, an antifouling enzyme, etc.) that may catalyze a reaction. For example, an enzyme may promote cleavage of a chemical bond in a biological cell wall, a viral proteinaceous molecule, and/or a cellular membrane component (e.g., a viral envelope component). In other embodiments, an antimicrobial proteinaceous molecule (e.g., a peptide) may possess a biostatic and/or a biocidal activity (e.g., activity via cell membrane permeablization). An antibiological proteinaceous molecule (e.g., a peptide) may compromise a cellular membrane (e.g., the cell membrane enclosing the cytoplasm, a viral envelope) to allow for cell wall and/or viral proteinaceous molecule disruption. These types of antibiological activities (e.g., an antimicrobial activity, an antifouling activity) may promote cell and/or virus lysis; promote ease of access to an inner structure of the cell and/or the virus (e.g., cytoplasm, an interior enzyme, an organelle component) by an antibiological agent; or a combination thereof, as the cell wall, viral proteinaceous molecule, and/or the cellular membrane becomes weaker (e.g., permeabilized). Improved access to an inner component of a cell and/or a virus may enhance the effectiveness of one or more antibiological agents (e.g., an antimicrobial agent, an antifouling agent, an enzyme, a peptide, a chemical preservative, etc.). For example, an enzymatic antibiological agent (e.g., an antimicrobial agent) may comprise a hydrolytic enzyme, such as a lysozyme that may cleave a peptidoglycan cell wall component. In another example, a lysozyme active in a coating may confer a catalytic, antimicrobial activity to a coating. In an alternative example, a lysozyme may be used in a material formulation such as a cream, an ointment, and/or a pharmaceutical, partly due to its size (14.4 kDa). In a further example, an antimicrobial peptide, ProteCoat™, may be efficacious against a Gram positive organism, and a combination of an antimicrobial and/or an antifouling enzyme (e.g., a lysozyme) demonstrates activity against cell(s). For example, a material formulation comprising a lipolytic enzyme such as a phospholipase and/or a cholesterol esterase that acts to compromise the integrity of a cell membrane, may allow ease of access for one or more enzyme(s) that degrade cell wall and/or viral proteinaceous coat component(s), and/or a preservative to act in a biocidal and/or a biostatic function as well (e.g., acts against a cell component).

In many embodiments, an enzyme that possesses an antiobiological activity (e.g., an antimicrobial activity, an antifouling activity) comprises a hydrolase (EC 3). In specific embodiments, the enzyme comprises a glycosylase (EC 3.2). In more specific embodiments, the enzyme comprises a glycosidase (EC 3.2.1), which comprises an enzyme that hydrolyses an O-glycosyl compound, a S-glycosyl compound, or a combination thereof. In particular aspects, the glycosidase acts on an O-glycosyl compound, and examples of such an enzyme include a lysozyme, an agarase, a cellulose, a chitinase, or a combination thereof. In other embodiments, an antibiological enzyme (e.g., an antimicrobial enzyme, an anti-fouling enzyme) acts on a cell wall, a viral proteinaceous molecule, and/or a cellular membrane component, and examples of such enzymes include a lysozyme, a lysostaphin, a libiase, a lysyl endopeptidase, a mutanolysin, a cellulase, a chitinase, an α-agarase, an β-agarase, a N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyhasparagine amidase, a mannosyl-glycoprotein endo-β-N-acetylglucosaminidase, a l-carrageenase, a κ-carrageenase, a λ-carrageenase, an α-neoagaro-oligosaccharide hydrolase, an endolysin, an autolysin, a mannoprotein protease, a glucanase, a mannose, a zymolase, a lyticase. a lipolytic enzyme, or a combination thereof. A commercially available enzyme may be used, such as, for example, a Viscozyme L carbohydrase produced from an Aspergillus spp. (Novozymes).

a. Lysozymes

Lysozyme (EC 3.2.1.17; CAS registry number: 9001-63-2) has been also referred to in that art as “peptidoglycan N-acetylmuramoylhydrolase,” “1,4-N-acetylmuramidase,” “globulin G,” “globulin G1,” “L-7001,” “lysozyme g,” “mucopeptide glucohydrolase,” “mucopeptide N-acetylmuramoylhydrolase,” “muramidase,” “N,O-diacetylmuramidase,” and “PR1-lysozyme.” A lysozyme catalyzes the reaction: in a peptidoglycan, hydrolyzes a (1,4)-β-linkage between N-acetylmuramic acid and a N-acetyl-D-glucosamine; in a chitodextrin (a polymer of (1,4)-β-linked N-acetyl-D-glucosamine monomers), hydrolyzes the (1,4)-β-linkage. A lysozyme demonstrates endo-N-acetylmuramidase activity, and may cleave a glycan comprising linked peptides, but has little or no activity toward a glycan that lack linked peptide. In many embodiments, a lysozyme comprises a single chain protein with a MW of 14.3kD. Lysozyme producing cells and methods for isolating a lysozyme from a cellular material and/or a biological source have been described [see, for example, Blade, C. C. F. et al., 1967a; Blake, C. C. F. et al., 1967b; Jolles, P., 1969; Rupley, J. A., 1964; Holler, H., et al., 1975; Canfield, R. E., 1963; Davies, R. C., et al., 1969), and may be used in conjunction with the disclosures herein. A common example of a lysozyme comprises a chicken egg white lysozyme (“CEWL”). The general activity range of a CEWL lysozyme may comprise about pH 6.0 to about 9.0, with maximal activity of the lysozyme at about pH 6.2 may be at an ionic strength of about 0.02 M to about 0.100 M, while at about pH 9.2 the maximal activity may be between an ionic strength of about 0.01 M to about 0.06 M. Another example of a lysozyme comprises a commercially available lysozyme (e.g., Sigma Aldrich).

Lysozymes comprise proteins with similar folding structures, generally divided into 9 classes. Four classes are noted for having particular effectiveness in cleaving a peptidoglycan: a bacteriophage T4 lysozyme, a goose egg-white lysozyme, a hen egg-white lysozyme, and a Chaloropsis lysozyme. Two domains connected by an alpha helix form the active site, with a glutamic acid located in the N-terminal half of the protein, in the C-terminal end of an alpha-helix. Another active site residue typically comprises an aspartic acid. An example of a Chalaropsis lysozyme comprises a cellosyl, which differs in having an active site comprising a single, flattened ellipsoid domain with a beta/alpha fold with a long groove comprising an electronegative hole on the C-terminal face. A cellosyl may be produced from Streptomyces coelicolor. An additional Chalaropsis lysozyme comprises LytC produced from Streptomyces pneumonia. Examples of an autolytic lysozyme include a SF muramidase from an Enterococus faecium (“Enterococcus hirae”; ATCC 9790); and/or a pesticin, encoded by the pst gene on the pPCP1 plasmid from Yersinia pestis. A lysozyme has been recombinantly expressed in Aspergillus niger (Gheshlaghi et al, 2005; Archer et al. 1990; Gyamerah et al. 2002; Mainwaring et al. 1999). Examples of modifications to a lysozyme include denaturation of the lysozyme, an attachment of a polysaccharide and/or a hydrophobic polypeptide to enhance effectiveness against a Gram negative bacterial, or a combination thereof (Touch et al., 2003; Aminlari et al., 2005; Ibrahim et al., 1994).

In some embodiments, a lysozyme damages and/or destroys a bacterial cell wall, and exemplifies an action many antimicrobial and/or antifouling enzymes. A lysozyme catalyzes cleavage of a peptidoglycan's glycosidic bond between a N-acetylmuramic acid (“NAM”) and a N-acetylglucosamine (“NAG”) that often comprise part of a cell wall. This glycosidic cross-link braces a relatively delicate cell membrane against a cell's high osmotic pressure. As a lysozyme acts, the structural integrity of the cell wall may be reduced (e.g., destroyed), and the bacteria cell bursts (“lysis”) under internal osmotic pressure. A lysozyme may act by an additional antimicrobial and/or antifouling mechanisms of action, other than enzymatic action, triggered by contact with a cell such as cell membrane damage, induction of an autolysin's activity, or a combination thereof (Masschalck and Michiels, 2003). In many embodiments, a lysozyme may be effective against a Gram positive bacteria since the peptidoglycan layer may be relatively accessible to the enzyme, although a lysozyme may be also effective against Gram negative bacteria that possess relatively less peptidoglycan in a cell wall, particularly after the outer membrane has been compromised, such as by contact with an anti-cellular membrane agent such as an antimicrobial and/or antifouling peptide, a detergent, a metal chelator (e.g., a metal ion chelator, EDTA), or a combination thereof.

Structural information for a wild-type lysozyme and/or a functional equivalent amino acid sequence for producing a lysozyme and/or a functional equivalent include Protein database bank entries: 102I, 103I, 104I, 107I, 108I, 109I, 110I, 111I, 112I, 113I, 114I, 115I, 116I, 118I, 119I, 120I, 122I, 123I, 125I, 126I, 127I, 128I, 129I, 130I, 131I, 132I, 133I, 134I, 135I, 137I, 138I, 139I, 140I, 141I, 142I, 143I, 144I, 145I, 146I, 147I, 148I, 149I, 150I, 151I, 152I, 153I, 154I, 155I, 156I, 157I, 158I, 159I, 160I, 161I, 162I, 163I, 164I, 165I, 166I, 167I, 168I, 169I, 170I, 171I, 1ior, 1ios, 1iot, 1ip1, 1ip2, 1ip3, 1ip4, 1ip5, 1ip6, 1ip7, 1ir7, 1ir8, 1ir9, 1ivm, 1iwt, 1iwu, 1iwv, 1iww, 1iwx, 1iwy, 1iwz, 1ixo, 1iy3, 1iy4, 1j1o, 1j1p, 1j1x, 1ja2, 1ja4, 1ja6, 1ja7, 1jef, 1jfx, 1jhl, 1jis, 1jit, 1jiy, 1jj0, 1jj1, 1jj3, 1jka, 1jkb, 1jkc, 1jkd, 1joz, 1jpo, 1jqu, 1jse, 1jsf, 1jtm, 1jtn, 1jto, 1jtp, 1jtt, 1jug, 1jwr, 1k28, 1kip, 1kiq, 1kir, 1kni, 1kqy, 1kqz, 1kr0, 1kr1, 1ks3, 1Kw5, 1Kw7, 1kxw, 1kxx, 1kxy, 1ky0, 1ky1, 1I00, 1I01, 1I02, 1I03, 1I04, 1I05, 1I06, 1I07, 1I08, 1I09, 1I0j, 1I0k, 1I10, 1I11, 1I12, 1I13, 1I14, 1I15, 1I16, 1I17, 1I18, 1I19, 1I20, 1I21, 1I22, 1I23, 1I24, 1I25, 1I26, 1I27, 1I28, 1I29, 1I30, 1I31, 1I32, 1I33, 1I34, 1I35, 1I36, 1I37, 1I38, 1I39, 1owz, 1oyu, 1p2c, 1p2l, 1p2r, 1p36, 1p37, 1p3n, 1p46, 1p56, 1p5c, 1p64, 1p6y, 1p7s, 1pdl, 1yil, 1ykx, 1yky, 1ykz, 1yl0, 1yl1, 1yqv, 1z55, 1zmy, 1zur, 1zv5, 1zvh, 1zvy, 1zwn, 1zyt, 200I, 201I, 205I, 206I, 207I, 208I, 209I, 210I, 211I, 212I, 213I, 214I, 215I, 216I, 217I, 2dqj, 2eiz, 2eks, 2epe, 2eql, 2f2n, 2f2q, 2f30, 2f32, 2f47, 2f4a, 2f4g, 2fbb, 2fbd, 2g4p, 2rbq, 2rbr, 2rbs, 2vb1, 2yss, 2yvb, 2z12, 2z18, 2z19, 2z2e, 2z2f, 2z6b, 3b61, 3b72, 3d3d, 3d9a, 3hfl, 3hfm, 3lhm, 3lym, 3lyo, 3lyt, 3lyz, 3lz2, 3lzm, 8lyz, 8lyz, 8lyz, 8lyz, 8lyz, 8lyz, 8lyz, 8lyz, 8lyz, 8lyz, 8lyz, 8lyz, 8lyz, 8lyz, 8lyz, 8lyz, and 8lyz. Examples of protein structure for lysozyme available in these entries include: a bacteriophage T4 lysozyme a from Escherichia coli expression; a mutant T4 lysozyme (e.g., a lysozyme comprising an engineered metal-binding site; an engineered thermostable lysozyme; a I99a; I99a and/or m102q mutant; a cavity producing mutants; an engineered salt bridge stability mutant; an engineered disulfide bond mutant; a g28a/i29a/g30a/c54t/c97a mutant; a 132a/133a/t34a/c54t/c97a/e108v; r14a/k16a/i17a/k19a/t21a/e22a/c54t/c97a mutant; a y24a/y25a/t26a/i27a/c54t/c97a mutant; a lysozyme comprising an alternative hydrophobic core packing of amino acids) sometimes from expression in Escherichia coli; a mutant (e.g., an i56t; an asp67his; a w64c; a c65a; a surface residue substitution; a N-terminal peptide addition; an i56t: a t152a; a t152c; a t152i; a t152s; a t152v; a v149c; a v149g; a v149i; a v149s; a synthetic lysozyme dimer; an unnatural amino acid p-iodo-1-phenylalanine at position 153; a mutant comprising an engineered calcium binding site) human lysozyme, sometimes from Spodoptera frugiperda, Saccharomyces cerevisiae, and/or Pichia pastoris expression; a Gallus gallus (chicken) lysozyme including a mutant form (e.g., a d52s), including from Escherichia coli and/or Saccharomyces cerevisiae expression; a Colinus virginianus (Bobwhite quail) lysozyme; a guinea-fowl lysozyme; a bacteriophage p22 lysozyme mutant (e.g., 187m) from Escherichia coli expression; a Cygnus atratus (black swan goose) lysozyme; a canine lysozyme from Pichia pastoris expression; a Mus musculus lysozyme expressed in an Escherichia coli; a bacteriophage p22 mutant (e.g., 186m) from Escherichia coli expression; a Streptomyces coelicolor lysozyme; a turkey lysozyme; and/or an Equus caballus lysozyme; etc.

Nucleotide and protein sequences for a lysozyme from various organisms are available via database such as, for example, KEGG. Examples of lysozyme and/or a functional equivalent KEEG sequences for production of wild-type and/or a functional equivalent nucleotide and protein sequence include: HSA-4069(LYZ); PTR-450190(LYZ); MCC-718361(LYZ); MMU-17105(Lyz2) 17110(Lyz1); RNO-25211(Lyz2); DPO-Dpse_GA11118 Dpse_GA20595; AGA-AgaP_AGAP005717 AgaP_AGAP007343 AgaP_AGAP007344 AgaP_AGAP007345 AgaP_AGAP007347 AgaP_AGAP007385; AAG-AaeL_AAEL003712 AaeL_AAEL003723 AaeL_AAEL005988 AaeL_AAEL009670 AaeL_AAEL010100 AaeL_AAEL015404; DBMO-Bmb021130; TCA-658610(LOC658610); ECC-c1436 c1562(ybcS) c3180 c4109(chiA); ECI-UTI89_C1303(ybcS1) UTI89_C1490 UTI89_C2660 UTI89_C3793(yheB) UTI89_C5112(ybcS2); ECP-ECP_{—}1160; ECV-APECO1_{—}1029 APECO1_{—}2033(ydfQ) APECO1_{—}242(ybcS2) APECO1_{—}3115(yheB) APECO1_{—}392 APECO1_{—}4196 APECO1_{—}514; ECW-EcE24377_A0827; ECX-EcHS_A0304 EcHS_A0931 EcHS_A1644; ECM-EcSMS35_{—}1183; ECL-EcolC_{—}2083 EcolC_{—}2770; STY-STY2044 STY3682(nucD) STY4620(nucD2); STT-t3424(nucD) t4314(nucD); XFT-PD0996(lycV) PD1113; XFM-Xfasm12_{—}0912 XfasM12_{—}1158; XFN-XfasM23_{—}1053 XfasM23_{—}1178; XAC-XAC1063(p13); XOP-PXO_{—}00139 PXO_{—}00141; SML-Smlt1054 Smlt1851 Smlt1944; SMT-Smal_{—}2511; VCO-VC0395_{—}1046; VHA-VIBHAR_{—}01975; PAP-PSPA7_{—}0693 PSPA7_{—}5063; PPG-PputGB1_{—}3388; PAR-Psyc_{—}1032; ABM-ABSDF0706 ABSDF1811; SON-SO_{—}0659; SDN-Sden_{—}3256; SFR-Sfri_{—}1671; SBL-Sbal_{—}1293 Sbal_{—}3605; SBM-Shew185_{—}2082; SBN-Sbal195_{—}0780 Sbal195_{—}2129; SDE-Sde_{—}2761; LSA-LSA1788; LSL-LSL_{—}0296 LSL_{—}0304 LSL_{—}0797 LSL_{—}0805 LSL_{—}1310; LRE-Lreu_{—}1367 Lreu_{—}1853; LRF-LAR_{—}1286; LFE-LAF_{—}1820; OOE-OEOE_{—}1199; CAC-CAC0554(lyc); CNO-NT01CX_{—}2099; CBA-CLB_{—}2952; CBT-CLH_{—}0905 CLH_{—}2072; SEN-SACE_{—}3764 SACE_{—}7138; SYG-sync_{—}1433 sync_{—}1864; SYX-SynWH7803_{—}0779; MAR-MAE_{—}54690; ANA-alr1167; AVA-Ava_{—}4421; PMF-P9303_{—}18641; TER-Tery_{—}4180; AMR-AM1_{—}0818; CCH-Cag_{—}0702; and/or PPH-Ppha_{—}0875Protein.

b. Lysostaphins

Lysostaphin (EC 3.4.24.75; CAS registry number: 9011-93-2) has been also referred to in that art as “glycyl-glycine endopeptidase.” Lysostaphin catalyzes the reaction: in a staphylococcal (e.g., S. aureus) peptidoglycan, hydrolyzes a-GlyGly-bond in a pentaglycine inter-peptide link (e.g., cleaves the polyglycine cross-links in the peptidoglycan layer of the cell wall of a Staphylococcus sp.). A lysostaphin typically comprises a zinc-dependent, 25-kDa endopeptidase with an activity optimum of about pH 7.5. Lysostaphin producing cells (e.g., Staphylococcus simulans, ATCC 67080, 69764, 67079, 67076, and 67078) and methods for isolating a lysostaphin from a cellular material and/or a biological source have been described [see, for example, Recsei, P. A., et al., 1987; Thumm, G. and Götz, F. 1997; Trayer, H. R., and Buckley, C. E., 1970; Browder, H. P., et al., 19, 383, 1965; Baba, T. and Schneewind, 1996], and may be used in conjunction with the disclosures herein. An example of a lysostaphin comprises a commercially available lysostaphin (e.g., Sigma Aldrich).

Structural information for a wild-type lysostaphin and/or a functional equivalent amino acid sequence for producing a lysostaphin and/or a functional equivalent include Protein database bank entries: 1QWY, 2B0P, 2B13, and/or 2B44. Examples of a lysostaphin and/or a functional equivalent KEEG sequences for production of wild-type and/or a functional equivalent nucleotide and protein sequence include: HAR: HEAR2799; SAU: SA0265(lytM); SAV: SAV0276(lytM); SAW: SAHV_{—}0274(lytM); SAM: MW0252(lytM); SAR: SAR0273(lytM); SAS: SAS0252; SAC: SACOL0263(lytM); SAB: SAB0215(lytM); SAA: SAUSA300_{—}0270(lytM); SAX: USA300HOU_{—}0289(lytM); SAO: SAOUHSC_{—}00248; SAJ: SaurJH9_{—}0260; SAH: SaurJH1_{—}0267; SAE: NWMN_{—}0210(lytM); NPU: Npun_F1058 Npun_F4149 Npun_F4637 Npun_F5024 Npun_F6078; AVA: Ava_{—}0183 Ava_{—}2410 Ava_{—}3195 Ava_{—}4756 Ava_{—}4929 Ava_C0210; AMR: AM1_{—}4073 AM1_{—}5374 and/or AM1_B0175.

c. Libiases

Libiase comprises an enzyme obtained from Streptomyces fulvissimus (e.g., Streptomyces fulvissimus TU-6) that it typically used to promote the lysis of Gram-positive bacteria (e.g., a Lactobacillus, an Aerococcus, a Listeria, a Pneumococcus, a Streptococcus). A libiase possesses a lysozyme and a β-N-acetyl-D-glucosaminidase activity, with activity optimum of about pH 4, and a stability optimum of about pH 4 to about pH 8. Commercial preparations of a libiase are available (Sigma-Aldrich). Libiase producing cells and methods for isolating a libiase from a cellular material and/or a biological source have been described (see, for example, Niwa et al. 2005; Ohbuchi, K. et al., 2001), and may be used in conjunction with the disclosures herein.

d. Lysyl Endopeptidases

Lysyl endopeptidase (EC 3.4.21.50; CAS registry number: 123175-82-6) has been also referred to in that art as “Achromobacter lyticus alkaline proteinase I”; “Achromobacter proteinase I”; “achromopeptidase”; “lysyl bond specific proteinase”; and/or “protease I,” A lysyl endopeptidase catalyzes the peptide cleavage reaction: at a Lys, including -LysPro-. In many embodiments, the lysyl endopeptidase comprises a (trypsin family) family 51 peptidase. Lysyl endopeptidase producing cells and methods for isolating a lysyl endopeptidase from a cellular material and/or a biological source (e.g., Achromobacter lyticus-ATCC 21457; Lysobacter enzymogenes ATCC 29488, 29487, 29486, Pseudomonas aeruginosa-ATCC 29511, 21472) have been described (see, for example, Ahmed et al, 2003; Chohnan et al. 2002; Elliott, B. W. and Cohen, C. 1986; Ezaki, T. and Suzuki, S., 1982; Jekel, P. A., et al., 1983; L1 et al. 1998; Masaki, T. et al. 1981; Masaki, T. et al., 1981; Ohara, T. et al., 1989; Tsunasawa, S. et al., 1989), and may be used in conjunction with the disclosures herein.

An example of a lysyl endopeptidase comprises a 27 kDa “achromopeptidase” obtained from Achromobacter lyticus M497-1 that may be used to promote lysis of a Gram positive bacterium typically resistant to a lysozyme. The achromopeptidase has an activity optimum of about pH 8.5 to about pH 9, and an example of an achromopeptidase comprises a commercially available achromopeptidase (e.g., Sigma Aldrich; Wako Pure Chemical Industries, Ltd.). Structural information for a wild-type lysyl endopeptidase and/or a functional equivalent amino acid sequence for producing a lysyl endopeptidase and/or a functional equivalent include Protein database bank entries: larb and/or 1arc. Examples of a lysyl endopeptidase and/or a functional equivalent KEEG sequences for production of wild-type and/or a functional equivalent nucleotide and protein sequence include: SRU: SRU_{—}1622.

e. Mutanolysins

Mutanolysin (EC 3.4.99.-) comprises a 23 kD N-acetyl muramidase obtained from Streptomyces globisporus (e.g., ATCC 21553). A mutanolysin catalyzes the reaction: in a cell wall peptidoglycan-polysaccharide, cleavage of a N-acetylmuramyl-β(1-4)-N-acetylglucosamine bond. Examples of cells that mutanolysin acts on include Gram positive bacteria (e.g., a Listeria, a Lactobacillus, a Lactococcus).

Mutanolysin producing cells and methods for isolating a mutanolysin from a cellular material and/or a biological source have been described (see, for example, Assaf, N. A., and Dick, W. A., 1993; Calandra, G. B., and Cole, R. M., 1980; Fliss, I., et al., Biotechniques, 1991; Yokogawa, K., et al., 1975), and may be used in conjunction with the disclosures herein.

A mutanolysin's binding of a cell wall polymer uses carboxy terminal moiety(s) of the enzyme, so mutagenesis and/or truncation of those amino acids may effect binding and enzyme activity. An example of a mutanolysin comprises a commercially available mutanolysin (e.g., Sigma Aldrich).

f. Cellulases

Cellulase (EC 3.2.1.4; CAS registry number: 9012-54-8) has been also referred to in that art as “4-(1,3;1,4)-β-D-glucan 4-glucanohydrolase,” “1,4-(1,3;1,4)-β-D-glucan 4-glucanohydrolase,” “9.5 cellulase,” “alkali cellulase,” “avicelase,” “celluase A; cellulosin AP,” “celludextrinase,” “cellulase A 3,” “endo-1,4-β-D-glucanase,” “endoglucanase D,” “pancellase SS,” “β-1,4-endoglucan hydrolase,” and/or “β-1,4-glucanase.” Cellulase catalyzes the reaction: in a cellulose, endohydrolysis of a (1,4)-β-D-glucosidic linkage; in a lichenin, endohydrolysis of a (1,4)-β-D-glucosidic linkage; and/or in a cereal β-D-glucan, endohydrolysis of a (1,4)-β-D-glucosidic linkage. In additional aspects, a cellulase may possess the catalytic activity of: hydrolyse of a 1,4-linkage in a β-D-glucan also comprising a 1,3-linkage. Cellulase producing cells and methods for isolating a cellulase from a cellular material and/or a biological source have been described [see, for example, Datta, P. K., et al., 1963; Myers, F. L. and Northcote, D. H., 1959; Whitaker, D. R. et al., 1963; Hatfield, R. and Nevins, D. J., 1986; Inohue, M. et al., 1999], and may be used in conjunction with the disclosures herein. A commercially available cellulase preparation (e.g., Sigma-Aldrich), often comprises an additional enzyme retained and/or added during preparation, such as a hemicellulase, to aid digestion of cellulose comprising substrates.

Structural information for a wild-type cellulase and/or a functional equivalent amino acid sequence for producing a cellulase and/or a functional equivalent include Protein database bank entries: 1A39; 1A3H; 1AIW; 1CEC; 1CEM; 1CEN; 1CEO; 1CLC; 1CX1; 1DAQ; 1DAV; 1DYM; 1DYS; 1E5J; 1ECE; 1EDG; 1EG1; 1EGZ; 1F9D; 1F9O; 1FAE; 1FBO; 1FBW; 1FCE; 1G01; 1G0C; 1G87; 1G9G; 1G9J; 1GA2; 1GU3; 1GZJ; 1H0B; 1H11; 1H1N; 1H2J; 1H5V; 1H8V; 1HD5; 1HF6; 1IA6; 1IA7; 1IS9; 1J83; 1J84; 1JS4; 1K72; 1KFG; 1KS4; 1KS5; 1KS8; 1KSC; 1KSD; 1KWF; 1L1Y; 1L2A; 1L8F; 1LF1; 1NLR; 1OA2; 1OA3; 1OA4; 1OA7; 1OA9; 1OCQ; 1OJI; 1OJJ; 1OJK; 1OLQ; 1OLR; 1OVW; 1QHZ; 1Q10; 1Q12; 1TF4; 1TML; 1TVN; 1TVP; 1ULO; 1ULP; 1UT9; 1UU4; 1UU5; 1UU6; 1UWW; 1V0A; 1VJZ; 1VRX; 1W2U; 1W3K; 1W3L; 1WC2; 1WZZ; 2A39; 2A3H; 2BOD; 2BOE; 2BOF; 2BOG; 2BV9; 2BVD; 2BW8; 2BWA; 2BWC; 2CIP; 2CIT; 2CKR; 2CKS; 2DEP; 2E0P; 2E4T; 2EEX; 2EJ1; 2ENG; 2EO7; 2EQD; 2JEM; 2JEN; 2NLR; 20VW; 2QNO; 2UWA; 2UWB; 2UWC; 2V38; 2V3G; 3A3H; 3B7M; 3ENG; 3OVW; 3TF4; 4A3H; 4ENG; 4OVW; 4TF4; 5A3H; 6A3H; 7A3H; and/or 8A3H. Examples of a cellulase and/or a functional equivalent KEEG sequences for production of wild-type and/or a functional equivalent nucleotide and protein sequence include: DFRU: 144551(NEWSINFRUG00000162829) 157531(NEWSINFRUG00000148215) 180346(NEWSINFRUG00000163275); DBMO: Bmb020157; CNE: CNH00790; CNB: CNBL0740; DPCH: 121193(e_gwh2.5.359.1) 129325(e_gwh2.2.646.1) 139079(e_gww2.2.208.1); LBC: LACBIDRAFT_{—}294705 LACBIDRAFT_{—}311963; DDI: DDB_{—}0215351(celA) DDB_{—}0230001; DPKN: PK11_{—}3250w; ECO: b3531(bcsZ); ECJ: JW3499(bcsZ); ECD: ECDH10B_{—}3708(bcsZ); ECE: Z4946(yhjM); ECS: ECs4411; ECC: c4343(yhjM); ECI: UTI89_C4063(yhjM); ECP: ECP_{—}3631; ECV: APECO1_{—}2917(bcsZ); ECW: EcE24377A_{—}4019(bcsZ); ECM: EcSMS35_{—}3840(bcsZ); ECL: EcolC_{—}0186; STY: STY4183(yhjM); STT: t3900(yhjM); SPT: SPA3473(yhjM); SEK: SSPA3243; SPQ: SPAB_{—}04494; SEC: SC3551; SEH: SeHA_C3933(bcsZ); SEE: SNSL254_A3889(bcsZ); SEW: SeSA_A3812(bcsZ); SEA: SeAg_B3825(bcsZ); SED: SeD_A3993(bcsZ); SEG: SG3819(bcsZ); BCN: Bcen_{—}0898; BCH: Bcen2424_{—}1380; BCM: Bcenmc03_{—}1358; BAM: Bamb_{—}1259; BAC: BamMC406_{—}1292; BMU: Bmul_{—}1925; BMJ: BMULJ=01315(egl); BPS: BPSS1581(bcsZ); BPM: BURPS1710b_A0632(bcsZ); BPL: BURPSI106A_A2145; BPD: BURPS668_A2231; BTE: BTH_{—}110792; BPH: Bphy_{—}3254; BPY: Bphyt_{—}5838; PNU: Pnuc_{—}1167; BAV: BAV2628(bcsZ); AAV: Aave_{—}2102; LCH: Lcho_{—}2071 Lcho_{—}2344; AZO: azo2236(eglA); GSU: GSU2196; GME: Gmet_{—}2294; GUR: Gura_{—}3125; GBM: Gbem_{—}0763; PCA: Pcar_{—}1216(sgcX); MXA: MXAN_{—}4837(celA); MTC: MT0067(celA); MRA: MRA0064(celA1) MRA1100(celA2a) MRA_{—}1101(celA2b); MTF: TBFG_{—}10061 TBFG_{—}11111; MBO: Mb0063(celA1) Mb1119(celA2a) Mb1120(celA2b); MBB: BCG_{—}0093(celA1) BCG_{—}1149(celA2a) BCG_{—}1150(celA2b); MAV: MAV_{—}0326; MSM: MSMEG_{—}6752; AAS: Aasi_{—}0590; CCH: Cag_{—}0339; PLT: Plut_{—}0993; RRS: RoseRS_{—}0349; RCA: Rcas_{—}0232; CAU: Caur_{—}1697; HAU: Haur_{—}1902; EMI: Emin_{—}0354; DRA: DR_{—}0229; MBA: Mbar_A0214; MMA: MM_{—}0673; MBU: Mbur_{—}0712; MEM: Memar_{—}1505; MBN: Mboo_{—}1201; MSI: Msm_{—}0134; MKA: MK0383; AFU: AF1795(celM); HAL: VNG1498G(celM); HSL: OE3143R; HMA: rrnAC0799(cdIM); HWA: HQ2923A(celM); NPH: NP4306A(celM); PHO: PH1171 PH1527; PAB: PAB0437 PAB0632(celB-like); PFU: PF1547; TKO: TK0781; SMR: Smar_{—}0057; HBU: Hbut_{—}1154; PAI: PAE1385; PIS: P isl_{—}1432; PCL: Pcal_{—}0842; PAS: Pars_{—}0452; CMA: Cmaq_{—}0206 Cmaq_{—}0950; TNE: Tneu_{—}0542; TPE: Tpen_{—}0002 Tpen_{—}0177; and/or KCR: Kcr_{—}0883Kcr_{—}1258.

g. Chitinases

Chitinase (EC 3.2.1.14; CAS registry number: 9001-06-3) has been also referred to in that art as “(1→4)-2-acetamido-2-deoxy-β-D-glucan glycanohydrolase,” “1,4-β-poly-N-acetylglucosaminidase,” “chitodextrinase,” “poly[1,4-(N-acetyl-β-D-glucosaminide)]glycanohydrolase,” “poly-β-glucosaminidase,” and/or “β-1,4-poly-N-acetyl glucosamidinase.” A chitinase catalyzes the reaction: random hydrolysis of a N-acetyl-β-D-glucosaminide (1→4)-β-linkage in a chitin; and random hydrolysis of a N-acetyl-β-D-glucosaminide (1→4)-β-linkage in a chitodextrin. In additional aspects, a chitinase may possess the catalytic activity of a lysozyme. Chitinase producing cells and methods for isolating a chitinase from a cellular material and/or a biological source have been described [see, for example, Fischer, E. H. and Stein, E. A. Cleavage of O- and S-glycosidic bonds (survey), in Boyer, P. D., Lardy, H. and Myrbäck, K. (Eds.), The Enzymes, 2nd end., vol. 4, pp. 301-312, 1960; Tracey, M. V., 1955], and may be used in conjunction with the disclosures herein. An example of a chitinase comprises a commercially available chitinase (e.g., Sigma Aldrich).

Structural information for a wild-type chitinase and/or a functional equivalent amino acid sequence for producing a chitinase and/or a functional equivalent include Protein database bank entries: 1CNS; 1CTN; 1D2K; 1DXJ; 1E6Z; 1ED7; 1EDQ; 1EHN; 1EIB; 1FFQ; 1FFR; 1GOI; 1GPF; 1H0G; 1H0I; 1HKI; 1HKJ; 1HKK; 1HKM; 1HVQ; 1ITX; 1K85; 1K9T; 1KFW; 1KQY; 1KQZ; 1KR0; 1KR1; 1LL4; 1LL6; 1LL7; 1LLO; 1NH6; 1O6I; GB; 1OGG; 1RD6; 1UR8; 1UR9; 1W1P; 1W1T; 1W1V; 1W1Y; 1W9P; 1W9U; 1W9V; 1WAW; 1WB0; 1WNO; 1WVU; 1WVV; 1X6L; 1X6N; 2A3A; 2A3B; 2A3C; 2A3E; 2CJL; 2CWR; 2CZN; 2D49; 2 DBT; 2DKV; 2DSK; 2HVM; 21UZ; 2UY2; 2UY3; 2UY4; 2UY5; 2Z37; 2Z38; 2Z39; 3B8S; 3B9A; 3B9D; 3B9E; 3CH₉; 3CHC; 3CHD; 3CHE; 3CHF; and/or 3CQL. Examples of a chitinase and/or a functional equivalent KEEG sequences for production of wild-type and/or a functional equivalent nucleotide and protein sequence include: HSA: 1118(CHIT1) 27159(CHIA); PTR: 457641(CHIT1); MCC: 703284(CHIA) 703286(CHIT1); MMU: 71884(Chit1) 81600(Chia); CFA: 479904(CHIA); BTA: 282645(CHIA); DECB: 100065255(LOC100065255); MDO: 100015954(LOC100015954) 100030396(LOC100030396) 100030417(LOC100030417) 100033109(LOC100033109) 100033117(LOC100033117) 100033119(LOC100033119); OAA: 100089089(LOC100089089); GGA: 395072(CHIA); XLA: 444170(MGC80644); XTR: 448265(chit1); TCA: 641592(Chi-3) 641601(Chi-1) 652967(Cht10) 655022(Idgf4) 655122(Idgf2) 656175(LOC656175) 658736(LOC658736) 660881(Cht7) 661383(Cht4) 661428(Cht8) 661938(LOC661938); CEL: CO₄F6.3(cht-1); CBR: CBG14201; BMY: Bml_{—}17035; ATH: AT3G12500(ATHCHIB) AT3G54420(ATEP3) AT5G24090; PPP: PHYPADRAFT_{—}138151 PHYPADRAFT_{—}153222 PHYPADRAFT_{—}219988 PHYPADRAFT_{—}52893 PHYPADRAFT_{—}55609; DOTA: Ot10g03210; CRE: CHLREDRAFT_{—}113089; SCE: YLR286C(CTS1); DSRD: 15784; DSMI: 15288; DSBA: 16756 26379; KLA: KLLA0C04730g; DKWA: Kwal_{—}23320; DHA: DEHA0F18073g DEHA0G06655g DEHA0G09636g; PIC: PICST_{—}31390(CHT4) PICST_{—}48142(CHT2) PICST_{—}68871(CHT3) PICST_{—}91537(CHT1); VPO: Kpol_{—}1009p7Kpol_{—}1062p25; CGR: CAGL0A02904g CAGL0M09779g; YLI: YALI0D22396g YALI0F04532g; NCR: NCU01393 NCU02184 NCU03026 NCU03209 NCU04500 NCU04554; PAN: PODANSg09468 PODANSg1191 PODANSg3325 PODANSg3488 PODANSg4492 PODANSg5997 PODANSg6135 PODANSg7650 PODANSg8762; YPG: YpAngola_A2570; YPI: YpsIP31758_{—}0611 YpsIP31758_{—}1757; YPY: YPK_{—}0693 YPK_{—}1864; YPB: YPTS_{—}3503; SSN: SSON_{—}1501(ydhO); ESA: ESA_{—}02015; KPN: KPN_{—}01993(ydh0); CKO: CKO_{—}02217; SAE: NWMN_{—}0931; LMF: LMOf2365_{—}0123(chiB); LWE: lwe0093; LLM: llmg_{—}2199(chiC); LBR: LVIS_{—}1777; CPR: CPR_{—}0949; CTH: Cthe_{—}0270; MMI: MMAR_{—}2010 MMAR_{—}2951; SGR: SGR_{—}2458; ART: Arth_{—}1229; AAU: AAur_{—}3218; TFU: Tfu_{—}0580 Tfu_{—}0868; ACE: Acel_{—}1458 Acel_{—}1460 Acel_{—}2033; SEN: SACE_{—}2232(chiB) SACE_{—}3887(chiC) SACE_{—}5287(chiC) SACE_{—}6557 SACE_{—}6558; STP: Strop_{—}4405; SAQ: Sare_{—}3672; OTE: Oter_{—}0638 Oter_{—}3591; CTA: CTA_{—}0134(ydh0); CTB: CTL0382; CTL: CTLon_{—}0378; SRU: SRU_{—}2812; and/or HAU: Haur_{—}2750.

h. α-Agarases

α-agarase (EC 3.2.1.158; CAS no. 63952-00-1) has been also referred to in that art as “agarose 3-glycanohydrolase,” “agarase,” and/or “agaraseA33.” α-agarase catalyzes the reaction: in an agarose, endohydrolysis of a 1,3-α-L-galactosidic linkage, producing an agarotetraose. Porphyran, a sulfated agarose, may also be cleaved. In additional aspects, an α-agarase obtained from a Thalassomonas sp. may possess the catalytic activity on a substrate such as a neoagarohexaose (“3,6-anhydro-α-L-galactopyranosyl-(1,3)-D-galactose”) and/or an agarohexaose. α-agarase activity may be enhanced by Ca²⁺. α-agarase producing cells and methods for isolating an α-agarase from a cellular material and/or a biological source have been described (see, for example, Ohta, Y., et al., 2005; Potin, P., et al., 1993), and may be used in conjunction with the disclosures herein.

i. β-Aqarases

β-agarase (EC 3.2.1.81; CAS registry number: 37288-57-6) has been also referred to in that art as “agarose 4-glycanohydrolase,” “AgaA,” “AgaB,” “agarase,” “agarose 3-glycanohydrolase,” and/or “endo-[3-agarase.” A β-agarase catalyzes the reaction: in agarose, hydrolysis of a 1,4-β-D-galactosidic linkage, producing a tetramer. An AgaA derived from Zobellia galactanivorans produces a neoagarohexaose and a neoagarotetraose, while an AgaB produces a neoagarobiose and a neoagarotetraose. A β-agarase also cleaves a porphyran. β-agarase producing cells and methods for isolating a β-agarase from a cellular material and/or a biological source have been described (see, for example, Allouch, J., et al., 2003; Duckworth, M. and Turvey, J. R. 1969; Jam, M. et al., 2005; Ohta, Y. et al., 2004a; Ohta, Y. et al., 2004b; Sugano, Y. et al., 1993), and may be used in conjunction with the disclosures herein. Structural information for a wild-type β-agarase and/or a functional equivalent amino acid sequence for producing a β-agarase and/or a functional equivalent include Protein database bank entries: 1O4Y, 1O4Z, and/or 1URX. Examples of a β-agarase and/or a functional equivalent KEEG sequences for production of wild-type and/or a functional equivalent nucleotide and protein sequence include: PPF: Pput_{—}1162; PAT: Patl_{—}1904 Patl_{—}1971 Patl_{—}2341 Patl_{—}2640 Patl_{—}2642; SDE: Sde_{—}1175 Sde_{—}1176 Sde_{—}2644 Sde_{—}2650 Sde_{—}2655; RPB: RPB_{—}3029; RPD: RPD_{—}2419; RPE: RPE_{—}4620; SCO: SCO3471(dagA); and/or RBA: RB3421(agrA).

j. N-Acetylmuramoyl-L-Alanine Amidases

N-acetylmuramoyl-L-alanine amidase (EC 3.5.1.28; CAS registry number: 9013-25-6) has been also referred to in that art as “peptidoglycan amidohydrolase,” “acetylmuramoyl-alanine amidase,” “acetylmuramyl-alanine amidase,” “acetylmuramyl-L-alanine amidase,” “murein hydrolase,” “N-acetylmuramic acid L-alanine amidase,” “N-acetylmuramoyl-L-alanine amidase type I,” “N-acetylmuramoyl-L-alanine amidase type II,” “N-acetylmuramylalanine amidase,” “N-acetylmuramyl-L-alanine amidase,” and/or “N-acylmuramyl-L-alanine amidase” A N-acetylmuramoyl-L-alanine amidase catalyzes the reaction: hydrolysis of a link between a L-amino acid residue and a N-acetylmuramoyl residue in some cell-wall glycopeptides. N-acetylmuramoyl-L-alanine amidase producing cells and methods for isolating a N-acetylmuramoyl-L-alanine amidase from a cellular material and/or a biological source have been described [see, for example, Ghuysen, J.-M. et al. 1969; Herbold, D. R. and Glaser, L. 1975; Ward, J. B. et al., 1982), and may be used in conjunction with the disclosures herein. Structural information for a wild-type N-acetylmuramoyl-L-alanine amidase and/or a functional equivalent amino acid sequence for producing a N-acetylmuramoyl-L-alanine amidase and/or a functional equivalent include Protein database bank entries: 1ARO, 1GVM, 1H8G, 1HCX, 1J3G, 1JWQ, 1LBA, 1X60, 1XOV, 2AR3, 2BGX, 2BH7, and/or 2BML. Examples of acetylmuramoyl-L-alanine amidase and/or a functional equivalent KEEG sequences for production of wild-type and/or a functional equivalent nucleotide and protein sequence include: HSA: 114770(PGLYRP2) 114771(PGLYRP3) 57115(PGLYRP4) 8993(PGLYRP1); PTR: 455797(PGLYRP2) 737434(PGLYRP3) 737562(PGLYRP4); MCC: 714583(L00714583) 718287(PGLYRP2) 718480(L00718480); MMU: 21946(Pglyrp1) 242100(Pglyrp3) 57757(Pglyrp2); RNO: 295180(Pglyrp3b) 310611(Pglyrp4) 499658(Pglyrp3); CFA: 610405(PGLYRP2) 612209(PGLYRP1); BTA: 282305(PGLYRP1) 510803(PGLYRP2) 532575(PGLYRP3); SSC: 396557(pPGRP-LB) 397213(PGLYRP1); GGA: 693263(PGRPL); XLA: 496035(L00496035); ECW: EcE24377A_{—}0941(amiD) EcE24377A_{—}2721(amiA); ECX: EcHS_A0971(amiD) EcHS_A2572(amiA) EcHS_A2963(amiC) EcHS_A4411; SFL: SF0822 SF2488(amiA) SF2828 SF4324(amiB); SFX: S0863 S2636(amiA) S3025 S4592(amiB); SFV: SFV_{—}0855 SFV_{—}2487(amiA) SFV_{—}2895 SFV_{—}4327(amiB); SSN: SSON_{—}0853 SSON_{—}2524(amiA) SSON_{—}2974 SSON_{—}4354(amiB); SBO: SBO_{—}0800 SBO_{—}2460(amiA) SBO_{—}2707 SBO_{—}4287(amiB); PLU: plu0645(amiC) plu2790 plu4584(amiB); BUC: BU576(amiB); BAS: BUsg555(amiB); HSO: HS_{—}1082(amiB); XCV: XCV1630 XCV1812(amiC) XCV2603(amiC) XCV3978(ampD); XAC: XAC1589 XAC1780(amiC) XAC2406(amiC) XAC3860; XOO: XOO2368(amiC) XOO2445 XOO2733(amiC) XOO4100; VFI: VF2326; SAE: NWMN_{—}0309 NWMN_{—}1035 NWMN_{—}1534 NWMN_{—}1773 NWMN_{—}1881; SEP: SE0750 SE1313; SPS: SPs0332; EFA: EF1293(ply-1) EF1486(ply-2); CAC: CAC0686 CAC3092(231); RCA: Rcas_{—}0212; HAU: Haur_{—}0094 Haur_{—}3648 Haur_{—}4245; EMI: Emin_{—}0232 Emin_{—}1374; RSD: TGRD_{—}681; TLE: Tlet_{—}1670; PMO: Pmob_{—}0199; and/or MMA: MM_{—}2290

k. Lytic Transqlycosylases

A lytic transglycosylase (“lytic murein transglycosylase,” EC 3.2.1.-) demonstrates exo-N-acetylmuramidase activity, and can cleave a glycan strand comprising linked a peptide and/or a glycan strand that lack linked peptides with similar efficiency. A lysozyme and a lytic transglycosylase cleaves the β1,4-glycosidic bond between a N-Acetyl-D-Glucosamine (“GlcNAc”) and a N-Acetylmuramic acid (“MurNAc”), but a lytic transglycosylase has a transglycosylation reaction producing a 1,6-anhydro ring at the MurNAc. A lytic transglycosylase may be inhibited by a N-acetylglucosamine thiazoline. An example of a lytic transglycosylase includes a MltB produced from Psudomonas aeruginosa. A lytic transglycosylase generally may be classified as a family 1, a family 2 (e.g., MltA), a family 3 (e.g., MltB) or a family 4 lytic transglycosylase (i.e., generally bacteriophage), based on a similar amino acid sequence, particularly comprising a conserved amino acid. A family 1 lytic transglycosylase may be classified as a 1A type (e.g., Slt70), a 1B type (e.g., MltC), a 1C type (e.g., EmtA), a 1D type (e.g., MltD), or a 1E type (e.g., YfhD). Lytic transglycosylase producing cells and methods for isolating a lytic transglycosylase from a cellular material and/or a biological source have been described [see, for example, Holtje et al, 1975; Thunnissen et al. 1994; Scheurwater et al, 2007; Reid et al., 2004; Blackburn and Clark, 2001), and may be used in conjunction with the disclosures herein.

Crystal structures for various lytic transglycosylases include those for a Neisseria gonorrhoeae MltA and an E. coli MltA; an E. coli Slt70; a phage A lytic transglycosylase; and an E. coli Slt35 (Powell et al., 2006; van Straaten et al., 2005; van Straaten et al., 2007; van Asselt et al., 1999a; Thunnissen et al., 1994; Leung et al., 2001; van Asselt et al., 1999b). A lytic transglycosylase active site generally comprises a glutamic acid (e.g., a Glu162 of Slt35; a Glu478 of Slt70), with a relatively more hydrophobic active site than a goose egg white lysozyme. Another active site residue may comprise an aspartic acid (e.g., an Asp308 of MltA). Structural information for a wild-type lytic transglycosylase and/or a functional equivalent amino acid sequence for producing a lytic transglycosylase and/or a functional equivalent include Protein database bank entries: 1Q2R, 1Q2S, 2PJJ, 2PIC, 1QSA, 2PNW, 1QTE, 1QUS, 1QUT, 1QDR, 1SLY, 1D0K, 1D0L, 1D0M, 3BKH, 3BKV, and/or 2AE0. Examples of lytic transglycosylase and/or a functional equivalent KEEG sequences for production of wild-type and/or a functional equivalent nucleotide and protein sequence include: ECO: b2701(mltB); ECJ: JW2671(mltB); ECE: Z4004(mltB); ECS: ECs3558; ECC: c3255(mltB); YPY: YPK_{—}1464; YEN: YE1242(mltB); SFL: SF2724(mltB); SFX: 52915(mltB); SFV: SFV_{—}2804(mltB); SSN: SSON_{—}2845(mltB); SBO: SBO_{—}2817(mltB); SBC: SbBS512_E3176(mltB); SDY: SDY_{—}2897(mltB); ECA: ECA1083(mltB); ENT: Ent638_{—}3179; ACB: A1S_{—}2316; ABM: ABSDF1210(mltB); ABY: ABAYE1161; SON: SO_{—}1166; SDN: Sden_{—}0853; SFR: Sfri_{—}0697; SAZ: Sama_{—}2590; SBL: Sbal_{—}3277; CV1: CV_{—}1609(mltB); RSO: RSc0918(mltB); REU: Reut_A2556; REH: H16_A0808(mltB); RME: Rmet_{—}0732; BMA: BMA0417; BMV: BMASAVP1_A2561; BML: BMA10229_A0937; BMN: BMA10247_{—}0212; BXE: Bxe_A0991; BVI: Bcep1808_{—}0977; POL: Bpro_{—}3149; PNA: Pnap_{—}1216; AAV: Aave_{—}2160; AJS: Ajs_{—}2817; VEI: Veis_{—}2099; MPT: Mpe_A1242; HAR: HEAR2564(mltB); NEU: NE1033(mltB2); NET: Neut_{—}2477; YPM: YP_{—}3487(mltC); YPA: YPA_{—}0310(mltC); YPN: YPN_{—}3152(mltC); YPS: YPTB3226(mltC); YEN: YE3445(mltC); SFL: SF2960(mltC); SFX: 53163(mltC); SFV: SFV_{—}3022(mltC); SSN: SSON_{—}3233(mltC); SBO: SBO_{—}3027(mltC); ILO: IL0198(mltC); TCX: Tcr_{—}0080; AHA: AHA_{—}3789; ASA: ASA_{—}0511(mltC); BCI: BCI_{—}0477(mltC); HHE: HH1830(mltC); WSU: WS1277; DVU: DVU1536; DVL: Dvul_{—}1595; DDE: Dde_{—}1786; LIP: LI1174(mltC); ECO: b0211(mltD); ECJ: JW5018(mltD); ECE: Z0235(dniR); SBO: SBO_{—}0200(dniR); SBC: SbBS512_E0207(mltD); SDY: SDY_{—}0230(dniR); ECA: ECA3343(mltD); PLU: plu0939(mltD); SGL: SG0588; ENT: Ent638_{—}0745; CKO: CKO_{—}02972; SPE: Spro_{—}0908; VCH: VC2237; VCO: VCO395_A1829(mltD); SPC: Sputcn32_{—}1775; SSE: Ssed_{—}1988; SHE: Shewmr4_{—}2162; SHM: Shewmr7_{—}2239; SHN: Shewana3_{—}2370; SHW: Sputw3181_{—}2250; ILO: IL1698(dniR); CPS: CPS_{—}1998; NMN: NMCC_{—}1210; RSO: RSc1516(RS03787); REU: Reut_A2186; BPE: BP3214; BPA: BPP3837; BBR: BB4281; RFR: Rfer_{—}1461; DVU: DVU0041; DVL: Dvul_{—}2920; DDE: Dde_{—}3580; LIP: L10055(mltD); FJO: Fjoh_{—}0976; CTE: CT0979; CCH: Cag_{—}1379; CPH: Cpha266_{—}1087; PVI: Cvib_{—}0782; YPE: YP02438; YPK: y1898(mltE); YPM: YP_{—}2226(mltE1); YPA: YPA_{—}1782; YPN: YPN_{—}1892; YPS: YPTB2346; YEN: YE1901; ECI: UTI89_C5165(slt); ECP: ECP_{—}4778; SFL: SF4424(slt); SFX: 54695(slt); SFV: SFV_{—}4426(slt); SSN: SSON_{—}4542(slt); XOO: XOO_{—}0820(slt); XOM: XOO_{—}0746(XOO_{—}0746); VCH: VC0700; VCO: VCO395_A0230(slt); WU: VV1_{—}0490; VVY: VV0706; VPA: VP0552; VFI: VF0558; VHA: VIBHAR_{—}00998; PPR: PBPRA0641; SFR: Sfri_{—}2529; SAZ: Sama_{—}1895; SBL: Sbal_{—}2273; SLO: Shew_{—}2125; SPC: Sputcn32_{—}2105; SSE: Ssed_{—}1979; SHE: Shewmr4_{—}2111; SHM: Shewmr7_{—}1863; FTL: FTL_{—}0466; FTH: FTH_{—}0463(slt); FTN: FTN_{—}0496(slt); TCX: Tcr_{—}0924; AEH: Mlg_{—}1378; HHA: Hhal_{—}1135; ABO: ABO_{—}1587; BPS: BPSL0262; BPM: BURPS1710b_{—}0453(slt); BPL: BURPSI106A_{—}0269; BPD: BURPS668_{—}0257; BTE: BTH_{—}10233; PNU: Pnuc_{—}1999; RFR: Rfer_{—}1088; POL: Bpro_{—}0652; PNA: Pnap_{—}0527; AAV: Aave_{—}4203; ECE: Z4130(mltA); ECS: ECs3673(mltA); ECC: c3384(mltA); ECI: UTI89_C3186(mltA); ECP: ECP_{—}2796(mltA); YPK: y3159(mltA); YPM: YP_{—}2826(mltA); YPA: YPA_{—}0496(mltA); YPN: YPN_{—}2977(mltA); YPG: YpAngola_A3225(mltA); PLU: plu0648(mltA); BUC: BU458(mltA); BAS: BUsg442(mltA); ENT: Ent638_{—}3259(mltA); CKO: CKO_{—}04178; SPE: Spro_{—}3810; HIN: HI0117(mltA); HIT: NTHI0205(mltA); CBU: CBU_{—}1111; LPN: lpg1994; LPF: Ip11970(mltA); LPP: lpp1975(mltA); BCN: Bcen_{—}2567; BCH: Bcen2424_{—}0538; BAM: Bamb_{—}0443; BMU: Bmul_{—}2856; BPS: BPSL3046; BPM: BURPS1710b_{—}3570(mltA); BPL: BURPSI106A_{—}3578(mltA); BPD: BURPS668_{—}3551(mltA); BTE: BTH_{—}12905; PNU: Pnuc_{—}0151; PNE: Pnec_{—}0165; BPE: BP3268; BPA: BPP4152; BJA: b1r0643; BRA: BRADO0205; MAG: amb4542; MGM: Mmc1_{—}0484; and/or SYP: SYNPCC7002_A2370(mltA).

I. Glucan Endo-1,3-β-D-Glucosidases

Glucan endo-1,3-β-D-glucosidase (EC 3.2.1.39; CAS registry number: 9025-37-0) has been also referred to in that art as “3-β-D-glucan glucanohydrolase,” “(1→3)-β-glucan 3-glucanohydrolase,” “1,3-β-D-glucan 3-glucanohydrolase,” “1,3-β-D-glucan glucanohydrolase,” “callase,” “endo-(1,3)-β-D-glucanase,” “endo-1,3-β-D-glucanase,” “endo-1,3-β-glucanase,” “endo-1,3-β-glucosidase,” “kitalase,” “laminaranase,” “laminarinase,” “oligo-1,3-glucosidase,” and/or “β-1,3-glucanase.” A glucan endo-1,3-β-D-glucosidase catalyzes the reaction: hydrolysis of a (1,3)-β-D-glucosidic linkage in a (1,3)-β-D-glucan. In additional aspects, a glucan endo-1,3-β-D-glucosidase may possess the catalytic activity of hydrolyzing a laminarin, a pachyman, a paramylon, or a combination thereof, and also have a limited hydrolysis activity against a mixed-link (1,3-1,4)-β-D-glucan. A glucan endo-1,3-β-D-glucosidase may be useful against fungal cell walls. Glucan endo-1,3-β-D-glucosidase producing cells and methods for isolating a glucan endo-1,3-β-D-glucosidase from a cellular material and/or a biological source have been described [see, for example, Chesters, C. G. C. and Bull, A. T., 1963; Reese, E. T. and Mandels, M., 1959; Tsuchiya, D., and Taga, M., 2001; Petit, J., et al., 10:4-5, 1994], and may be used in conjunction with the disclosures herein. An enzyme preparation comprising a glucan endo-1,3-β-D-glucosidase prepared from a Rhizoctonia solani (“Kitalase”), or a Trichoderma harzianum (Glucanex®) (Sigma-Aldrich). Structural information for a wild-type glucan endo-1,3-β-D-glucosidase and/or a functional equivalent amino acid sequence for producing a glucan endo-1,3-β-D-glucosidase and/or a functional equivalent include Protein database bank entries: 1GHS, 2CYG, 2HYK, and/or 3DGT. Examples of an endo-1,3-β-D-glucosidase and/or a functional equivalent KEEG sequences for production of wild-type and/or a functional equivalent nucleotide and protein sequence include: DBMO: Bmb007310; ATH: AT3G57260(BGL2); DPOP: 769807(fgenesh4_pg.C_LG_X001297); MGR: MGG_{—}09733; TET: TTHERM_{—}00243770 TTHERM_{—}00637420 TTHERM_{—}00956460 TTHERM_{—}00956480; SFR: Sfri_{—}1319; SAZ: Sama_{—}1396; SDE: Sde_{—}3121; PIN: Ping_{—}0554; RLE: RL3815; MMR: Mmar10_{—}0247; NAR: Saro_{—}1608; SAL: Sala_{—}0919; RHA: RHA1_ro05769 RHA1_ro05771; and/or FJO: Fjoh_{—}2435.

m. Endo-1,3(4)-β-Glucanases

Endo-1,3(4)-β-glucanase (EC 3.2.1.6; CAS registry number: 62213-14-3) has been also referred to in that art as “3-(1→3;1→4)-β-D-glucan 3(4)-glucanohydrolase,” “1,3-(1,3;1,4)-β-D-glucan 3(4)-glucanohydrolase,” “endo-1,3-1,4-β-D-glucanase,” “endo-1,3-β-D-glucanase,” “endo-1,3-β-D-glucanase,” “endo-1,3-β-glucanase,” “endo-β-(1→3)-D-glucanase,” “endo-β-(1→3)-D-glucanase,” “endo-β-1,3(4)-glucanase,” “endo-β-1,3-1,4-glucanase,” “endo-β-1,3-glucanase IV,” “laminaranase,” “laminarinase,” 1,4-glucanase,” and/or “β-1,3-glucanase.” An endo-1,3(4)-β-glucanase catalyzes the reaction: endohydrolysis of a (1,3)-linkage in a β-D-glucan and/or a (1,4)-linkage in a β-D-glucan, wherein the hydrolyzed link's glucose residue is substituted at a C-3 of the reducing moiety that is part of the substrate chemical linkage. Endo-1,3(4)-β-glucanase producing cells and methods for isolating an endo-1,3(4)-β-glucanase from a cellular material and/or a biological source have been described [see, for example, Barras, D. R. and Stone, B. A., 1969a; Barras, D. R. and Stone, B. A., 1969b; Cunningham, L. W. and Manners, D. J., 1961; Reese, E. T. and Mandels, M., 1959; Soya, V. V., Elyakova, L. A. and Vaskovsky, V. E., 1970], and may be used in conjunction with the disclosures herein. Structural information for a wild-type endo-1,3(4)-β-glucanase and/or a functional equivalent amino acid sequence for producing an endo-1,3(4)-β-glucanase and/or a functional equivalent include Protein database bank entries: 1UP4, 1UP6, 1UP7, and/or 2CL2. Examples of an endo-1,3(4)-β-glucanase and/or a functional equivalent KEEG sequences for production of wild-type and/or a functional equivalent nucleotide and protein sequence include: NCR: NCU04431 NCU07076; PAN: PODANSg699 PODANSg9033; FGR: FG04768.1 FG06119.1 FG08757.1; AFM: AFUA_{—}1G04260 AFUA_{—}1G05290 AFUA_{—}3G03080 AFUA_{—}4G13360; AFUA_{—}5G02280 AFUA_{—}5G13990 AFUA5G14030 AFUA_{—}6G14540; ANG: An01g03090; DPCH: 10833(fgeneshi_pm.C_scaffold_{—}14000004) 123909(e_gwh2.6.417.1); LBC: LACBIDRAFT174636 LACBIDRAFT191735 LACBIDRAFT_{—}250640; LACBIDRAFT_{—}255995; PFA: PFL0285w; PFH: PFHG_{—}03986; PYO: PY001776; DPKN: PK12_{—}0440w; BCL: ABC2683 ABC2776; 01H: OB2143; CBE: Cbei_{—}2710; HWA: HQ2923A(celM); and/or NPH: NP4306A(celM).

n. β-Lytic Metalloendopeptidases

β-lytic metalloendopeptidase (EC 3.4.24.32; CAS no. 37288-92-9) has been also referred to in that art as “achromopeptidase component,” “Myxobacter β-lytic proteinase,” “Myxobacter495 β-lytic proteinase,” “Myxobacterium sorangium β-lytic proteinase,” “β-lytic metalloproteinase,” and/or “β-lytic protease.” A β-lytic metalloendopeptidase catalyzes the reaction: a N-acetylmuramoyl Ala cleavage, as well as an insulin B chain cleavage. A β-lytic metalloendopeptidase may be used, for example, against a bacterial cell wall. β-lytic metalloendopeptidase producing cells and methods for isolating a β-lytic metalloendopeptidase from a cellular material and/or a biological source (e.g., an Achromobacter lyticus Lysobacter enzymogenes) have been described [see, for example, Whitaker, D. R. et al., 1965; Whitaker, D. R. and Roy, C., 1967; Li, S. L. et al., 1990; Altmann, F. et al., 1986; Plummer, T. H., Jr. and Tarentino, A. L., 1981; Takahashi, N., 1977; Takahashi, N. and Nishibe, H., 1978; Tarentino, A. L. et al., 1985.], and may be used in conjunction with the disclosures herein.

o. 3-Deoxy-2-Octulosonidases

3-deoxy-2-octulosonidase (EC 3.2.1.124; CAS no. 103171-48-8) has been also referred to in that art as “capsular-polysaccharide 3-deoxy-D-manno-2-octulosonohydrolase,” “2-keto-3-deoxyoctonate hydrolase,” “octulofuranosylono hydrolase,” “octulopyranosylonohydrolase,” and/or “octulosylono hydrolase.” A 3-deoxy-2-octulosonidase catalyzes the reaction: endohydrolysis of the β-ketopyranosidic linkage of a 3-deoxy-D-manno-2-octulosonate in a capsular polysaccharide. A 3-deoxy-2-octulosonidase acts on a polysaccharide of a bacterial (e.g., an Escherichia coli) cell wall. 3-deoxy-2-octulosonidase producing cells and methods for isolating a 3-deoxy-2-octulosonidase from a cellular material and/or a biological source have been described [see, for example, Altmann, F. et al., 1986], and may be used in conjunction with the disclosures herein.

p. Peptide-N4-(N-acetyl-β-Glucosaminyl)asparaqine Amidases

Peptide-N⁴-(N-acetyl-β-glucosaminyl)asparagine amidase (EC 3.5.1.52; CAS no. 83534-39-8) has been also referred to in that art as “N-linked-glycopeptide-(N-acetyl-β-D-glucosaminyl)-L-asparagine amidohydrolase,” “glycopeptidase,” “glycopeptide N-glycosidase,” “Jack-bean glycopeptidase,” “N-glycanase,” “N-oligosaccharide glycopeptidase,” “PNGase A,” and/or “PNGase F.” A peptide-N⁴-(N-acetyl-β-glucosaminyl)asparagine amidase catalyzes the reaction: hydrolysis of a N⁴-(acetyl-β-D-glucosaminyl)asparagine residue. The reaction may promote the glycosylation of the glyglucosamine residue, and produce a peptide comprising an aspartate and a substituted N-acetyl-β-D-glucosaminylamine. Peptide-N⁴-(N-acetyl-β-glucosaminyl)asparagine amidase does not substantively act on (GlcNAc)Asn, as 3 or more amino acids in the substrate promotes the reaction. Peptide-N⁴-(N-acetyl-β-glucosaminyl)asparagine amidase producing cells and methods for isolating an eptide-N⁴-(N-acetyl-β-glucosaminyl)asparagine amidase from a cellular material and/or a biological source have been described [see, for example, Plummer, T. H., Jr. and Tarentino, A. L., 1981; Takahashi, N. and Nishibe, H., 1978; Takahashi, N., 1977; Tarentino, A. L. et al., 1985], and may be used in conjunction with the disclosures herein. Structural information for a wild-type peptide-N⁴-(N-acetyl-β-glucosaminyl) asparagine amidase and/or a functional equivalent amino acid sequence for producing a peptide-N⁴-(N-acetyl-β-glucosaminyl)asparagine amidase and/or a functional equivalent include Protein database bank entries: 1PGS, 1PNF, 1PNG, 1X3W, 1X3Z, 2D5U, 2F4M, 2F4O, 2G9F, 2G9G, 2HPJ, 2HPL, and/or 2I74. Examples of peptide-N⁴-(N-acetyl-β-glucosaminyl)asparagine amidase and/or a functional equivalent KEEG sequences for production of wild-type and/or a functional equivalent nucleotide and protein sequence include: HSA: 55768(NGLY1); PTR: 460233(NGLY1); MCC: 700842(LOC700842); DECB: 100059456(LOC100059456); OAA: 100075786(LOC100075786); GGA: 420655(NGLY1); DRE: 553627(zgc:110561); DFRU: 139051(NEWSINFRUG00000131342); DTNI: 33706; DOLA: 10847(ENSORLG00000008647); DCIN: 289359(estExt_fgenesh3_pg.C_chr_{—}05q0441); DME: Dmel_CG7865(PNGase); DPO: Dpse_GA20643; AGA: AgaP_AGAP007390; AAG: AaeL_AAEL014507; DAME: 9653(ENSAPMG00000005556); DBMO: Bmb025391; TCA: 664307(LOC664307); BMY: Bml_{—}49720; ATH: AT5G49570(ATPNG1); DPOP: 241215(gw1.XIII.1464.1); DWI: GSVIVP00031149001(GSVIVT00031149001); OSA: 4343301(Os07g0497400); PPP: PHYPADRAFT_{—}151482; OLU: OSTLU_{—}5312; DOTA: Ot14g02360; CRE: CHLREDRAFT_{—}146964; DHA: DEHAOE22572g; VPO: Kpol_{—}1074p3; CGR: CAGLOH05753g; YLI: YALI0C23562g; NCR: NCU00651; FGR: FG01650.1; MBR: MONBRDRAFT_{—}8805; and/or DTPS: 35410(e_gw1.7.250.1).

q. Mannosyl-Glycoprotein Endo-3-N-Acetylglucosaminidases

Mannosyl-glycoprotein endo-β-N-acetylglucosaminidase (EC 3.2.1.96; CAS no. 37278-88-9) has been also referred to in that art as “glycopeptide-D-mannosyl-N⁴-(N-acetyl-D-glucosaminyl)-2-asparagine 1,4-N-acetyl-β-glucosaminohydrolase,” “di-N-acetylchitobiosyl β-N-acetylglucosaminidase,” “endoglycosidase S,” “endo-N-acetyl-β-D-glucosaminidase,” “endo-N-acetyl-β-glucosaminidase,” “endo-β-(1,4)-N-acetylglucosaminidase,” “endo-β-acetylglucosaminidase,” “endo-β-N-acetylglucosaminidase D,” “endo-β-N-acetylglucosaminidase F,” “endo-β-N-acetylglucosaminidase H,” “endo-β-N-acetylglucosaminidase L; “endo-β-N-acetylglucosaminidase,” “mannosyl-glycoprotein 1,4-N-acetamidodeoxy-β-D-glycohydrolase,” “mannosyl-glycoprotein endo-β-N-acetylglucosamidase,” and/or “N,N′-diacetylchitobiosyl β-N-acetylglucosaminidase.” A mannosyl-glycoprotein endo-β-N-acetylglucosaminidase catalyzes the reaction: a N,N′-diacetylchitobiosyl unit endohydrolysis in a high-mannose glycoprotein and/or a glycopeptide comprising a -[Man(GlcNAc)₂]Asn-structure, wherein the intact oligosaccharide is released and a N-acetyl-D-glucosamine residue is still attached to the protein. Mannosyl-glycoprotein endo-β-N-acetylglucosaminidase producing cells and methods for isolating a mannosyl-glycoprotein endo-β-N-acetylglucosaminidase from a cellular material and/or a biological source have been described [see, for example, Chien, S., et al., 1977; Koide, N. and Muramatsu, T., 1974; Pierce, R. J. et al., 1979; Pierce, R. J. et al., 1980; Tai, T. et al., 1975; Tarentino, A. L., et al., 1974.], and may be used in conjunction with the disclosures herein. Structural information for a wild-type mannosyl-glycoprotein endo-β-N-acetylglucosaminidase and/or a functional equivalent amino acid sequence for producing a mannosyl-glycoprotein endo-β-N-acetylglucosaminidase and/or a functional equivalent include Protein database bank entries: 1C3F, 1C8X, 1C8Y, 1C90, 1C91, 1C92, 1C93, 1EDT, 1EOK, 1EOM, and/or 2EBN. Examples of mannosyl-glycoprotein endo-β-N-acetylglucosaminidase and/or a functional equivalent KEEG sequences for production of wild-type and/or a functional equivalent nucleotide and protein sequence include: HSA: 64772(FLJ21865); OAA: 100089364(LOC100089364); DCIN: 254322(gw1.55.22.1); DAME: 24424(ENSAPMG00000015707) 33583(ENSAPMG00000015707); DBMO: Bmb029819; TCA: 658146(LOC658146); BMY: Bml_{—}17595; DHA: DEHA0F20174g; PIC: PICST_{—}32069(HEX1); MBR: MONBRDRAFT_{—}34057; TBR: Tb09.160.2050; BCL: ABC3097; LSP: Bsph_{—}1040; SAU: SA0905(atl); SAV: SAV1052; SAW: SAHV_{—}1045; SAM: MW0936(atl); SAR: SAR1026(atl); SAS: SAS0988; SAC: SACOL1062(atl); SHA: SH1911(atl); SSP: SSP1741; LLM: 11 mg_{—}1087(acmC) 11 mg_{—}2165(acmB); SPZ: M5005_Spy_{—}1540(endoS); SPH: MGAS10270_Spy1607(endoS); SPI: MGAS10750_Spy1599(endoS); SPJ: MGAS2096_Spy1565(endoS); SPK: MGAS9429_Spy1544(endoS); SPF: SpyM50309; SPA: M6_Spy1530; SPB: M28_Spy1527(endoS); LBR: LVIS_{—}1883; 00E: OEOE_{—}0144; CNO: NT01CX_{—}0726; CBA: CLB_{—}3142; BLJ: BLD_{—}0197; and/or CHU: CHU_{—}1472(flgJ).

r. l-Carraqeenases

l-carrageenase (EC 3.2.1.157) has been also referred to in that art as “l-carrageenan 4-β-D-glycanohydrolase (configuration-inverting).” An l-carrageenase catalyzes the reaction: in an l-carrageenan, endohydrolysis of a 1,4-β-D-linkage between a 3,6-anhydro-D-galactose-2-sulfate and a D-galactose 4-sulfate. l-carrageenase producing cells and methods for isolating an l-carrageenase from a cellular material and/or a biological source have been described [see, for example, Barbeyron, T. et al., 2000; Michel, G. et al., 2001; Michel, G. et al., 2003], and may be used in conjunction with the disclosures herein. Structural information for a wild-type l-carrageenase and/or a functional equivalent amino acid sequence for producing a l-carrageenase and/or a functional equivalent include Protein database bank entries: 1H80 and/or 1KTW.

s. κ-Carraqeenases

κ-carrageenase (EC 3.2.1.83; CAS no. 37288-59-8) has been also referred to in that art as “κ-carrageenan 4-β-D-glycanohydrolase,” “κ-carrageenan 4-β-D-glycanohydrolase (configuration-retaining).” κ-carrageenase catalyzes the reaction: in a κ-carrageenans, endohydrolysis of a 1,4-β-D-linkage between a 3,6-anhydro-D-galactose and a D-galactose 4-sulfate. κ-carrageenase often acts against an algae (e.g., red algae). κ-carrageenase producing cells and methods for isolating a κ-carrageenase from a cellular material and/or a biological source have been described [see, for example, Weigl, J. and Yashe, W., 1966; Potin, P. et al., 1991; Potin, P. et al., 1995; Michel, G. et al., 1999; Michel, G., et al., 2001.], and may be used in conjunction with the disclosures herein. Structural information for a wild-type κ-carrageenase and/or a functional equivalent amino acid sequence for producing a κ-carrageenase and/or a functional equivalent include Protein database bank entries: 1DYP. Examples of κ-carrageenase and/or a functional equivalent KEEG sequences for production of wild-type and/or a functional equivalent nucleotide and protein sequence include: RBA: RB2702.

t. λ-Carraqeenases

λ-carrageenase (EC 3.2.1.162) has been also referred to in that art as “endo-(1→4)-λ-carrageenose 2,6,2′-trisulfate-hydrolase,” and/or “endo-β-1,4-carrageenose 2,6,2′-trisulfate-hydrolase.” A λ-carrageenase catalyzes the reaction: in a λ-carrageenan, endohydrolysis of a (1,4)-β-linkage, producing a α-D-Galp-2,6S2-(1,3)-β-D-Galp2S-(1,4)-α-D-Galp-2,6S2-(1,3)-D-Galp2S tetrasaccharide. λ-carrageenase producing cells and methods for isolating a λ-carrageenase from cellular materials (e.g., Pseudoalteromonas sp) and biological sources have been described [see, for example, Ohta, Y. and Hatada, 2006], and may be used in conjunction with the disclosures herein.

u. α-Neoaqaro-Oligosaccharide Hydrolases

α-neoagaro-oligosaccharide hydrolase (EC 3.2.1.159) has been also referred to in that art as “α-neoagaro-oligosaccharide 3-glycohydrolase,” “α-neoagarooligosaccharide hydrolase,” and/or “α-NAOS hydrolase.” An α-neoagaro-oligosaccharide hydrolase catalyzes the reaction: hydrolysis of a 1,3-α-L-galactosidic linkage in a neoagaro-oligosaccharide, wherein the substrate is a pentamer or smaller, producing a D-galactose and a 3,6-anhydro-L-galactose. α-neoagaro-oligosaccharide hydrolase producing cells and methods for isolating a NAME from a cellular material and/or a biological source have been described [see, for example, Sugano, Y., et al. 1994], and may be used in conjunction with the disclosures herein.

v. Additional Antibiological Enzymes

An endolysin may be used for a Gram positive bacteria, such as one that may be resistant to a lysozyme. An endolysin comprises a phage encoded enzyme that fosters release of a new phage by destruction of a cell wall. An endolysin may comprise a N-acetylmuramidase, a N-acetylglucosamimidae, an emdopeptidase, and/or an amidase. An endolysin may be translocated by phage encoded holin protein in disrupting a cytosolic membrane (Wang et al., 2000). A LysK lysine from phage k and a Listeria monocytogenes bacteriophage-lysin have been recombinantly expressed in a Lactoccus lactus and/or an E. coli (Loessner et al. 1995; Gaeng et al. 2000; O'Flaherty et al. 2005). An autolysin such as, for example, from Staphylococcus aureus, Bacillus subtilis, or Streptococcus pneumonia, may also be used as an antimicrobial and/or an antifouling enzyme (Smith et al, 2000; Lopez et al. 2000; Foster et al. 1995).

A protease may be used to cleave the mannoprotein outer cell wall layer, such as for a fungi such as a yeast. A glucanase such as, for example, a beta(1->6) glucanase, a glucan endo-1,3-β-D-glucosidase, and/or an endo-1,3(4)-β-glucanase can then more easily cleave glucan from the inner cell wall layer(s). Combinations of a protease and a glucanase may be used to produce an improved lytic activity. A reducing agent, such as a dithiothreitol of beta-mercaptoethanol, may aid in allowing enzyme contact with the inner cell wall by breaking a disulfide linkage, such as between a cell wall protein and a mannose. A mannose, a chitinase, a proteinase, a pectinase, an amylase, or a combination thereof may also be used, such as for aiding cell wall component cleavage. Examples of enzymes that degrade fungal cell walls include those produced by an Arthrobactersp., a Celluloseimicrobium cellulans (“Oerskovia xanthineolytica LL G109”) (DSM 10297), a Cellulosimicrobium cellulans (“Arthobacter luaus 73/14”) (ATCC 21606), a Cellulosimicrobium cellulans TK-1, a Rarobacter faecitabidus, a Rhizoctonia sp., or a combination thereof. An Arthrobacter sp. produces a protease with a functional optimum of about pH 11 and about 55° C. (Adamitsch et al., 2003). A Celluloseimicrobium cellulans (ATCC 21606) produces a protease and a glucanase (“lyticase”) with a functional optimum of about pH 10 and about pH 8.0, respectively (Scott and Schekman, 1980; Shen et al., 1991). A Celluloseimicrobium cellulans (DSM 10297) produces a protease with functional optimums of about pH 9.5 to about pH 10, and a glucanase with a functional optimum of about pH 8.0 and about 40° C. (Salazar et al. 2001; Ventom and Asenjo, 1990). A Rarobacter faecitabidus produces a protease effective against cell wall a component (Shimoi et al, 1992). A Rarobacter sp. produces a glucanase with a functional optimum of about pH 6 to about pH 7, and about 40° C. (Kobayashi et a1.1981). In specific aspects, commercially available enzyme preparations such as a zymolase and/or a lyticase (Sigma-Aldrich), generally comprising a β-1,3-glucanase and another enzyme, may be used.

2. Antibiological Peptides and Polypeptides

Additional examples of an antibiological proteinaceous molecule include the peptide sequences described in U.S. Pat. Nos. 6,020,312; 5,885,782; and 5,602,097, and patent application Ser. Nos. 10/884,355 and 11/368,086, and these antibiological peptides (e.g., antifungal peptides) include those of SEQ ID No. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or a combination thereof. For example, SEQ ID Nos. 1-47, which comprise sequences from a peptide library, may be used individually (e.g., SEQ ID No. 14, SEQ ID No. 41), or in a combination (e.g., a mixture of SEQ ID Nos. 25-47). These sequences establish a number of precise chemical compositions which possess antibiological (e.g., antifungal) activity. For example, one or more of these proteinaceous sequences may be used against a spectrum of fungi. One or more of these sequences may be useful, for example, in a material formulation and/or an application for an antibiological proteinaceous composition (e.g., for treating and/or protecting building materials and other non-living objects from infestation by a cell such as a fungi). For ease of reference, a proteinaceous molecule (e.g., a peptide) herein are written in the C-terminal to N-terminal direction to denote the sequence of synthesis. However, the conventional N-terminal to C-terminal manner of reporting amino acid sequences is utilized in the Sequence Listings. In some embodiments, a sequence may be produced and used in the forward and/or reverse pattern (e.g., synthesized C-terminal to N-terminal manner, or the reverse N-terminal to C-terminal). In some embodiments, a relatively variable composition (e.g., “XXXXRF”; SEQ ID No. 1) may be described as, for example, an antibiological peptide (e.g., an antifungal peptide), even though it may be possible that not every peptide encompassed by that general sequence possesses the same or any antibiological (e.g., antifungal) activity.

A proteinaceous composition (e.g., a peptide composition) may exhibit variable abilities to, for example, prevent and/or inhibit growth (e.g., fungal growth) as adjudged by the minimal inhibitory concentrations (MIC mg/ml) and/or the concentrations necessary to inhibit growth of fifty percent of a population of cells (e.g., a fungal spore, a cell, a mycelia) (1050 mg/ml). For example, in certain aspects, the MICs may range depending upon the proteinaceous additive (e.g., a peptide additive comprising one or more SEQ ID Nos. 1 to 199) and target organism from about 3 to about 1700 mg/ml (e.g., about 3 to about 300 mg/ml), while the IC₅₀'s may range depending upon the proteinaceous additive (e.g., a peptide additive) and target organisms from about 2 to about 1700 mg/ml (e.g., about 2 to about 100 mg/ml). Target organisms susceptible to these amounts include, for example, a Fusarium oxysporum, a Fusariam Sambucinum, a Rhizoctonia Solani, a Ceratocystis Fagacearum, a Pphiostoma ulmi, a Pythium ultimum, a Magaporthe Aspergillus nidulans, an Aspergillus fumigatus, and/or an Aspergillus Parasiticus. For example, a peptide (e.g., an antifungal peptide) of about 8 to about 10 amino acid residues long also has the property of inhibiting the growth of bacteria, including disease-causing bacteria such as a Staphalococcus and a Streptococcus. In a further example, a peptide sequence such as SEQ ID Nos. 6, 7, 8, 9, and/or 10, may act on a cell such as a bacteria and a fungi. In a specific example, a peptide sequence such as SEQ ID Nos. 41, 197, 198, and 199, can inhibit growth of an Erwinia amylovora, an Erwinia carotovora, an Escherichia coli, an Ralstonia solanocerum, an Staphylococcus aureus, and/or an Streptococcus faecalis in standard media at IC50's of between about 10 to about 1100 mg/ml and MIC's of between about 20 to about 1700 mg/ml.

For the purposes of preparing and using a proteinaceous molecule as an active antibiological agent (e.g., an antifungal agent), such as an antibiological agent used in a material formulation (e.g., a paint, a coating composition), it may not be necessary to understand the mechanism by which the desired antibiological (e.g., an antifungal) effect is exerted on a cell and/or a virus. However, possible modes of action of a peptide, a polypeptide, and/or a protein, by which they exert their effect(s) (e.g., an inhibitory effect, a fungicidal effect), may include, for example, destabilizing a cellular (e.g., a fungal cell) membrane (e.g., perturb membrane functions responsible for osmotic balance); a disruption of macromolecular synthesis (e.g., cell wall biosynthesis) and/or metabolism; disruption of appressorium formation; or a combination thereof. (see, for example, Fiedler, H. P., et al. 1982; Isono, K. and S. Suzuki. 1979; Zasloff, M. 1987; U.S. patent application Ser. No. 10/601,207).

For example, a proteinaceous composition may comprise one or more peptide(s), polypeptide(s), and/or protein(s) (e.g., an enzyme, an antimicrobial enzyme, an anti-cell wall enzyme, an anti-cell membrane enzyme). For example, one or more peptide(s) and enzyme(s) may be selected for a mixture due to related activity(s) (e.g., antibiological activity). In some embodiments, a proteinaceous composition (e.g., a peptide composition) comprises a substantially homogeneous proteinaceous composition, and/or a mixture of proteinaceous molecules (e.g., a plurality of peptides). For example, a homogeneous peptide composition may comprise a single active peptide specie of a well-defined sequence, though a minor amount (e.g., less than about 20% by moles) of impurity(s) may coexist with the peptide in the peptide composition so long as the impurity does not interfere with a desired property(s) of the active peptide (e.g., a growth inhibitory property). In certain instances, a peptide may have a completely defined sequence. For example, an antifungal peptidic agent may comprise a single peptide of a precise sequence (e.g., the hexapeptide of SEQ ID No. 198, SEQ ID No. 41, SEQ ID No. 197, SEQ ID No. 198, SEQ ID No. 199, etc.). However, it is not necessary for a proteinaceous composition (e.g., a peptide), that may possess a demonstrable activity (e.g., antibiotic activity, antifungal activity), to be completely defined as to each residue. For example, an alternative to using one or more isolated antifungal peptides as a peptide composition (e.g., an antifungal peptidic agent), the peptide composition may instead comprise a mixture of peptides (e.g., an aliquot of a peptide library, a mixture of isolated peptides). In such an example, the peptide composition comprising a mixture of peptides may comprise at least one active peptide (e.g., a peptide having antifungal activity). In another example, a peptide composition may comprise an active (e.g., an antifungal) peptide, wherein the peptide composition may be impure to the extent that the peptide composition may comprise one or more peptides of unknown exact sequence which may or may not have activity (e.g., an antifungal activity). In a further example, a mixed proteinaceous composition (e.g., a mixed peptide composition) may be used treat a target (e.g., a biological target, a fungal target, a viral target) with lower concentrations of numerous active additives (e.g., a plurality of active peptides, a plurality of antifungal peptides) rather than a higher concentration of a single chemical composition (e.g., a single peptide sequence); a mixed proteinaceous composition may be used to treat an array of targets (e.g., a plurality of target organisms, a plurality of fungal organisms) each with a different causative agent; or combination thereof. In certain embodiments, a proteinaceous (e.g., a peptide mixture, a synthetic peptide combinatorial library) comprises an equimolar mixture of proteinaceous molecules (e.g., an equimolar mixture of peptides). In some embodiments, at least one (e.g., 1, 2, 3, 4, 5, 6, or more such as to about 10,000 amino acids) of the amino acid residue(s) (e.g., an N-terminal amino acid residue, a C-terminal amino acid residue) is known for proteinaceous molecule (e.g., a peptide) in a proteinaceous molecule mixture (e.g., a peptide mixture such as a peptide library). For example, the peptidic agent may comprise a peptide library aliquot comprising a mixture of peptides in which at least two, three and/or four or more of the N-terminal amino acid residues are known. In some aspects wherein one or more amino acid residues(s) are known for a proteinaceous molecule (e.g., a peptide) in a mixture, the amino acid residue(s) may be in common for a plurality of proteinaceous molecules (e.g., for each peptide) in the mixture. In some aspects, a mixed proteinaceous composition (e.g., a mixed peptide composition) comprises one or more variable amino acid residue(s), and such a proteinaceous molecule mixture (e.g., a peptide mixture, a peptide library) may be selected for use due to the increased cost of testing and/or the cost of producing a completely defined proteinaceous molecule (e.g., an defined antibiotic peptide).

For example, the sequence of a peptide (e.g., an antifungal peptide) may be defined for only certain of the C-terminal amino acid residues leaving the remaining amino acid residues defined as equimolar ratios. For example, certain of the peptides of SEQ ID Nos. 1 to 199 have somewhat variable amino acid compositions. Thus, in certain aspects, in each aliquot of the SPCL comprising a given SEQ ID Nos. having a variable residue, the variable residue(s) may each be uniformly represented in equimolar amounts by one of nineteen different naturally-occurring amino acids in one or the other stereoisomeric form. However, the variable residue(s) may be rapidly defined using the method described in one or more of U.S. Pat. Nos. 6,020,312; 5,885,782; and 5,602,097, and patent application Ser. Nos. 10/884,355 and 11/368,086 to identify peptide(s) that possess activity (e.g., controlling fungal growth). In the cited patents it was demonstrated that peptides encompassed by the C-terminal sequence “XXXXRF” (SEQ ID No. 1) exhibited antifungal activity for a wide spectrum of fungi.

In another example of peptide assaying and screening, for the identification of antifungal peptides encompassed by the general sequence “XXXXRF” (SEQ ID No. 1) parent composition of antifungal activity, “XXXLRF” (SEQ ID No. 9) peptides mixtures were found to exhibit antibiotic activity (also disclosed in U.S. Pat. Nos. 6,020,312; 5,885,782; and 5,602,097, and patent application Ser. Nos. 10/884,355 and 11/368,086). Similarly to the parent composition “XXXXRF” (SEQ ID No. 1), the “XXXLRF” (SEQ ID No. 9) peptides may have a mixed equimolar array of peptides representing the same nineteen amino acid residues, some of which may have antibiological (e.g., antifungal activity) and some of which may not have such activity. Overall, however, the “XXXLRF” (SEQ ID No. 9) peptide composition comprises an antibiological (e.g., an antifungal agent). This process may be carried out to the point where completely defined peptide(s) are produced and assayed for antibiological (e.g., antifungal) activity. As a result, and as was accomplished for the representative peptide “FHLRF” (SEQ ID No. 31), all amino acid residues in a six residue peptide may be known.

A proteinaceous composition may also be non-homogenous, comprising, for example, both D-, L- and/or cyclic amino acids. In many embodiments, a proteinaceous composition comprises a plurality (e.g., a mixture) of different proteinaceous molecules, including proteinacous molecule(s) that comprise an L-amino acid, a D amino acid, a cyclic amino acid, or a combination thereof. For example, a mixture of different proteinaceous molecules may comprises one or more peptides comprising L amino acids; one or more peptides comprising D amino acids; and/or one or more peptides comprising both an L amino acid and an D-amino acid. For example, a retroinversopeptidomimetic of SEQ ID No. (41) demonstrated inhibitory function, albeit less so than either the D- or L-configurations, against certain household fungi such as a Fusarium and an Aspergillus (Guichard, 1994).

In some aspects, a peptide composition may comprise or be modified to comprises fewer cysteines and/or exclude cysteine(s) to reduce and/or prevent disulfide linkage problem that may occur in certain facets (e.g., a product). In some aspects, one or more peptides may be prepared as a peptide library, which typically comprises a plurality (e.g., about 2 to about 10¹⁰ peptides). A peptide library may comprise a D-amino acid, an L-amino acid, a cyclic amino acid, a common amino acid, an uncommon amino acid (e.g., a non-naturally occurring amino acid), a stereoisomer (e.g., a D-amino acid stereoisomer, an L-amino acid stereoisomer), or a combination thereof. A peptide library may comprise a synthetically produced peptide and/or a biologically produced peptide (e.g., a recombinantly produced peptide, see for example U.S. Pat. No. 4,935,351). For example, a synthetic peptide combinational library (“SPCL”) typically comprises a mixture (e.g., an equimolar mixture) of free peptide(s).

A SPCL peptide may possess activity (e.g., an antifungal activity, antipathogen activity), such as, for example, a SPCL comprising 52,128,400 six-residue peptides, wherein each peptide comprised D-amino acids and having non-acetylated N-termini and amidated C-termini. As described in U.S. Pat. Nos. 6,020,312; 5,885,782; and 5,602,097, and patent application Ser. Nos. 10/884,355 and 11/368,086, a hexapeptide library comprised peptides with the first two amino acids in each peptide chain individually and specifically defined and with the last four amino acids comprising an equimolar mixtures of 20 amino acids. Four hundred (400) (20²) different peptide mixtures each comprising 130,321 (19⁴)(cysteine was eliminated) individual hexamers were evaluated. In such a peptide mixture, the final concentration for each peptide was about 9.38 ng/ml in a mixture comprising about 1.5 mg (peptide mix)/ml solution. This mixture profile assumed that an average peptide has a molecular weight of about 785. This concentration was sufficient to permit testing for antifungal activity. In some embodiments, an antibiotic composition(s) comprising equimolar mixture of peptides produced in a synthetic peptide combinatorial library (see U.S. Pat. Nos. 6,020,312; 5,885,782; and 5,602,097, and patent application Ser. Nos. 10/884,355 and 11/368,086,) have been derived and shown to have desirable antibiotic activity. In certain embodiments, these relatively variable compositions are based upon the sequences of one or more of the peptides disclosed in any of the U.S. Pat. Nos. 6,020,312; 5,885,782; and 5,602,097, and patent application Ser. Nos. 10/884,355 and 11/368,086.

In some embodiments, a peptide composition comprises a peptide derived from amino acids of a length readily accomplished using standard peptide synthesis procedures, such as, for example, between about 3 to about 100 amino acids in length (e.g., about 3 to about 25 residues in length, about 6 residues in length, etc.). In other embodiments, a proteinaceous molecule (e.g., an antifungal peptide sequence identified as described herein) may be grown in suitable cell(s) (e.g., a bacterial cell, an insect cell) employing recombinant techniques and materials described herein and/or of the art, using DNA encoding the proteinaceous molecule's sequence (e.g., encoding an antifungal peptide's sequence described herein) which may be used instead of and/or in combination with a previous DNA sequence. For example, an expression vector may comprise a DNA sequence encoding SEQ ID No. 1 in the correct orientation and reading frame with respect to the promoter sequence to allow translation of the DNA encoding the SEQ ID No. 1. Examples of such cloning and expression of an exemplary gene and DNAs are described herein and in the art. As described herein and in the art, such a proteinaceous sequence, whether synthetically and/or recombinantly produced, may comprise one or more other sequences (e.g., extracellular and/or intracellular signal sequence(s) to target a proteinaceous molecule, restriction enzyme site(s), ion and/or metal binding sites such as a His-Tag), for ease of processing, preparation, and/or to alter and/or confer an additional property. For example, a plurality of peptide sequence(s), which may comprise multiple copies of the same and/or different sequences, may be produced. One or more restriction enzyme site(s) may expressed between selected sequence(s), to allow cleavage into smaller proteinaceous molecules (e.g., cleavage into smaller peptide sequences). A metal binding site such as a His-tag may be added for ease of purification and/or to confer a metal binding property. Thus, a peptide sequence may be included as part of a polypeptide by incorporation of one or more copies of peptide sequence(s), additional sequences (e.g., His-tags, restriction enzyme sites). Further, one or more peptide sequence(s) and/or one or more such additional sequences may be added to the C-terminus and/or the N-terminus of another proteinacous sequence (e.g., an enzyme). For example, an enzyme (e.g., an antibiological enzyme, an esterase) may be modified to comprise an antimicrobial peptide sequence, a restriction enzyme site, and/or a metal binding domain (e.g., a His-Tag), with the additional proteinaceous sequence(s) added at the N-terminus, the C-terminus, or a combination thereof.

In some embodiments, a proteinaceous composition (e.g., an antibiotic proteinaceous composition, an antibiotic peptide) may comprise a carrier (e.g., a microsphere, a liposome, a saline solution, a buffer, a solvent, a soluble carrier, an insoluble carrier). In certain aspects, the carrier may be one suitable for a permanent, a semi-permanent, and/or a temporary material formulation (e.g., a permanent surface coating application, a semi-permanent coating, a non-film forming coating, a temporary coating). In many embodiments, a carrier may be selected to comprise a chemical and/or a physical characteristic which does not significantly interfere with the antibiotic activity of a proteinaceous (e.g., a peptide) composition. For example, a microsphere carrier may be effectively utilized with a proteinaceous composition in order to deliver the composition to a selected site of activity (e.g., onto a surface). In another example, a liposome may be similarly utilized to deliver an antibiotic (e.g., a labile antibiotic). In a further example, a saline solution, a material formulation (e.g., a coating) acceptable buffer, a solvent, and/or the like may also be utilized as a carrier for a proteinaceous (e.g., a peptide) composition.

3. Antbiological Agent Targets

An antibiological agent (e.g., an antimicrobial agent, an antifouling agent) may act on a biological entity such as a biological cell and/or a biological virus. Examples of a cell include a prokaryotic cell and/or an eukaryotic cell. An antibiological agent generally binds a biomolecule ligand to act on the biological entity, such as, for example an enzyme cleaving a cellular biomolecule and/or a peptide associating with and disrupting a cellular membrane.

a. Cells

Prokaryotic organisms are generally classified in the Kingdom Monera as an Archaea (“Archaebacteria”) or an Eubacteria (“bacteria”). Eukaryotic organisms are generally classified in the Kingdom Animalia (“animals”), the Kingdom Fungi (“fungi”), the Kingdom Plantae (“plants”) or the Kingdom Protista (“protists”). A virus does not possess a cell wall, but comprises a proteinaceous outer coat, that may be surrounded by a phospholipid membrane (“envelope”). In some aspects, a cell and/or a virus that may be a target of an antibiological agent comprises an Animalia cell (e.g., a mollusk cell), a Plantae cell, an Archaea cell, an Eubacteria cell, a Fungi cell, a Protista cell, a virus (e.g., an enveloped virus), or a combination thereof. In specific facets, a cell and/or a virus that may be a target of an antibiological agent may comprise a microorganism, a marine fouling organism, or a combination thereof. An antibiological proteinaceous composition may be referred to by the target cell it effects, such as an “antifungal peptidic agent.” In some embodiments, such a cell may comprise a pathogen (e.g., a fungal pathogen, a plant pathogen, an animal pathogen such as a human pathogen, etc.).

i. Archaea

An Archaea typically comprises a cell wall comprising a pseudopeptidoglycan, a peptide, a polypeptide, a protein (e.g., a glycoprotein), or a combination thereof. Examples of an Archaea genus includes an Acidianus, an Acidilobus, an Aeropyrum, an Archaeoglobus, a Caldivirga, a Desulfurococcus, a Ferroglobus, a Ferroplasma, a Haloarcula, a Halobacterium, a Halobaculum, a Halococcus, a Haloferax, a Halogeometricum, a Halomicrobium, a Halorhabdus, a Halorubrum, a Haloterrigena, a Hyperthermus, an Ignicoccus, a Metallosphaera, a Methanobacterium, a Methanobrevibacter, a Methanocalculus, a Methanocaldococcus, a Methanococcoides, a Methanococcus, a Methanocorpusculum, a Methanoculleus, a Methanofollis, a Methanogenium, a Methanohalobium, a Methanohalophilus, a Methanolacinia, a Methanolobus, a Methanomicrobium, a Methanomicrococcus, a Methanoplanus, a Methanopyrus, a Methanosaeta, a Methanosalsum, a Methanosarcina, a Methanosphaera, a Methanospirillum, a Methanothermobacter, a Methanothermococcus, a Methanothermus, a Methanothrix, a Methanotorris, a Natrialba, a Natronobacterium, a Natronococcus, a Natronomonas, a Palaeococcus, a Picrophilus, a Pyrobaculum, a Pyrococcus, a Pyrodictium, a Pyrolobus, a Staphylothermus, a Stetteria, a Stygiolobus, a Sulfolobus, a Sulfophobococcus, a Sulfurisphaera, a Thermococcus, a Thermofilum, a Thermoplasma, a Thermoproteus, a Thermosphaera, a Vulcanisaeta, or a combination thereof.

ii. Eubacteria

An Eubacteria typically comprises a cell wall comprising a peptidoglycan, a peptide, a polypeptide, a protein (e.g., a glycoprotein), a lipid, or a combination thereof. Often, the members of the Eubacteria phyla are divided into Gram-positive Eubacteria or Gram-negative Eubacteria (e.g., Cyanobacteria, Proteobacteria, Spirochetes) based on biochemical and structural differences between the cell wall and/or an associated a phospholipid bilayer (“cell membrane”) of the organism(s). A “Gram-positive Eubacteria” (“Gram-positive bacteria”) refers to an Eubacteria comprising a cell wall that typically stains positive with Gram stain reaction (see, for example, Scherrer, R., 1984) and may not be surrounded by an outer cell membrane. A Gram positive bacteria generally have a cell wall composed of a thick layer of peptidoglycan overlaid by a thinner layer of techoic acid. A “Gram-negative Eubacteria” (“Gram negative bacteria”) refers to Eubacteria comprising a cell wall that typically stains negative with Gram stain reaction and may be surrounded by a second lipid bilayer (“outer cell membrane”). Gram negative bacteria have a thinner layer of peptidoglycan. A few types of Gram-negative Eubacteria do not stain well using a standard Gram stain procedure. However, these bacteria may be classified as a Gram-negative Eubacteria by the presence of an outer cell membrane, a morphological feature typically not present in a Gram-positive Eubacteria.

Examples of a Gram-positive Eubacteria comprise an Acetobacterium, an Actinokineospora, an Actinomadura, an Actinomyces, an Actinoplanes, an Actinopolyspora, an Actinosynnema, an Aerococcus, an Aeromicrobium, an Agromyces, an Amphibacillus, an Amycolatopsis, an Arcanobacterium, an Arthrobacter, an Aureobacterium, a Bacillus, a Bifidobacterium, a Brachybacterium, a Brevibacterium, a Brochothrix, a Carnobacterium, a Caryophanon, a Catellatospora, a Cellulomonas, a Clavibacter, a Clostridium, a Coprococcus, a Coriobacterium, a Corynebacterium, a Curtobacterium, a Dactylosporangium, a Deinobacter, a Deinococcus, a Dermabacter, a Dermatophilus, a Desulfotomaculum, an Enterococcus, an Erysipelothrix, an Eubacterium, an Exiguobacterium, a Falcivibrio, a Frankia, a Gardnerella, a Gemella, a Geodermatophilus, a Glycomyces, a Gordonia, an Intrasporangium, a Jonesia, a Kibdelosporangium, a Kineosporia, a Kitasatospora, a Kurthia, a Lactobacillus, a Lactococcus, a Leuconostoc, a Listeria, a Marinococcus, a Melissococcus, a Microbacterium, a Microbispora, a Micrococcus, a Micromonospora, a Microtetraspora, a Mobiluncus, a Mycobacterium, a Nocardia, a Nocardioides, a Nocardiopsis, an Oerskovia, a Pediococcus, a Peptococcus, a Peptostreptococcus, a Pilimelia, a Planobispora, a Planococcus, a Planomonospora, a Promicromonospora, a Propionibacterium, a Pseudonocardia, a Rarobacter, a Renibacterium, a Rhodococcus, a Rothia, a Rubrobacter, a Ruminococcus, a Saccharococcus, a Saccharomonospora, a Saccharopolyspora, a Saccharothrix, a Salinicoccus, a Sarcina, a Sphaerobacter, a Spirillospora, a Sporichthya, a Sporohalobacter, a Sporolactobacillus, a Sporosarcina, a Staphylococcus, a Streptoalloteichus, a Streptococcus, a Streptomyces, a Streptosporangium, a Syntrophosphora, a Terrabacter, a Thermacetogenium, a Thermoactinomyces, a Thermoanaerobacter, a Thermoanaerobium, a Thermomonospora, a Trichococcus, a Tsukamurella, a Vagococcus, or a combination thereof.

Examples of a Gram-negative Eubacteria comprises an Acetivibrio, an Acetoanaerobium, an Acetobacter, an Acetomicrobium, an Acidaminobacter, an Acidaminococcus, an Acidiphilium, an Acidomonas, an Acidovorax, an Acinetobacter, an Aeromonas, an Agitococcus, an Agrobacterium, an Agromonas, an Alcaligenes, an Allochromatium, an Alteromonas, an Alysiella, an Aminobacter, an Anabaena, an Anaerobiospirillum, an Anaerorhabdus, an Anaerovibrio, an Ancalomicrobium, an Ancylobacter, an Angulomicrobium, an Aquaspirillum, an Archangium, an Arsenophonus, an Arthrospira, an Asticcacaulis, an Azomonas, an Azorhizobium, an Azospirillum, an Azotobacter, a Bacteroides, a Bdellovibrio, a Beggiatoa, a Beijerinckia, a Blastobacter, a Blastochloris, a Bordetella, a Borrelia, a Brachyspira, a Bradyrhizobium, a Brevundimonas, a Brucella, a Budvicia, a Buttiauxella, a Butyrivibrio, a Calothrix, a Campylobacter, a Capnocytophaga, a Cardiobacterium, a Caulobacter, a Cedecea, a Cellulophaga, a Cellvibrio, a Centipeda, a Chitinophaga, a Chlorobium, a Chloroflexus, a Chlorogloeopsis, a Chloroherpeton, a Chondromyces, a Chromobacterium, a Chromohalobacter, a Chroococcidiopsis, a Citrobacter, a Cobetia, a Comamonas, a Crinalium, a Cupriavidus, a Cyclobacterium, a Cylindrospermum, a Cystobacter, a Cytophaga, a Dermocarpella, a Derxia, a Desulfobacter, a Desulfobacterium, a Desulfobulbus, a Desulfococcus, a Desulfomicrobium, a Desulfomonile, a Desulfonema, a Desulfosarcina, a Desulfovibrio, a Desulfurella, a Desulfuromonas, a Dichotomicrobium, an Ectothiorhodospira, an Edwardsiella, an Eikenella, an Enhydrobacter, an Ensifer, an Enterobacter, an Erwinia, an Erythrobacter, an Erythromicrobium, an Escherichia, an Ewingella, a Fervidobacterium, a Fibrobacter, a Filomicrobium, a Fischerella, a Flammeovirga, a Flavobacterium, a Flectobacillus, a Flexibacter, a Flexithrix, a Francisella, a Frateuria, a Fusobacterium, a Gemmata, a Gemmiger, a Gloeobacter, a Gloeocapsa, a Gluconobacter, a Haemophilus, a Hafnia, a Haliscomenobacter, a Haloanaerobium, a Halobacteroides, a Halochromatium, a Halomonas, a Halorhodospira, a Helicobacter, a Heliobacillus, a Heliobacterium, a Herbaspirillum, a Herpetosiphon, a Hirschia, a Hydrogenophaga, a Hyphomicrobium, a Hyphomonas, an Ilyobacter, an Isochromatium, an Isosphaera, a Janthinobacterium, a Kingella, a Klebsiella, a Kluyvera, a Labrys, a Lachnospira, a Lamprocystis, a Lampropedia, a Leclercia, a Legionella, a Leminorella, a Leptospira, a Leptospirillum, a Leptothrix, a Leptotrichia, a Leucothrix, a Lysobacter, a Malonomonas, a Marinilabilia, a Marichromatium, a Marinobacter, a Marinomonas, a Megamonas, a Megasphaera, a Melittangium, a Meniscus, a Mesophilobacter, a Metallogenium, a Methylobacillus, a Methylobacterium, a Methylococcus, a Methylomonas, a Methylophaga, a Methylophilus, a Methylovorus, a Microscilla, a Mitsuokella, a Moellerella, a Moraxella, a Morganella, a Morococcus, a Myxococcus, a Myxosarcina, a Nannocystis, a Neisseria, a Nevskia, a Nitrobacter, a Nitrococcus, a Nitrosococcus, a Nitrosomonas, a Nitrosospira, a Nitrospira, a Nostoc, an Obesumbacterium, an Oceanospirillum, an Ochrobactrum, an Oligella, an Oscillatoria, an Oxalobacter, a Pantoea, a Paracoccus, a Pasteurella, a Pectinatus, a Pedobacter, a Pedomicrobium, a Pelobacter, a Pelodictyon, a Persicobacter, a Phaeospirillum, a Phenylobacterium, a Photobacterium, a Phyllobacterium, a Pirellula, a Planctomyces, a Plesiomonas, a Pleurocapsa, a Polyangium, a Porphyrobacter, a Porphyromonas, a Pragia, a Prevotella, a Propionigenium, a Propionispira, a Prosthecobacter, a Prosthecochloris, a Prosthecomicrobium, a Proteus, a Providencia, a Pseudanabaena, a Pseudomonas, a Psychrobacter, a Rahnella, a Rhabdochromatium, a Rhizobacter, a Rhizobium, a Rhizomonas, a Rhodobacter, a Rhodobium, a Rhodoblastus, a Rhodobaca, a Rhodocista, a Rhodocyclus, a Rhodoferax, a Rhodomicrobium, a Rhodopila, a Rhodoplanes, a Rhodopseudomonas, a Rhodospirillum, a Rhodothalassium, a Rhodovibrio, a Rhodovulum, a Rikenella, a Roseobacter, a Roseococcus, a Rugamonas, a Rubrivivax, a Ruminobacter, a Runella, a Salmonella, a Saprospira, a Scytonema, a Sebaldella, a Selenomonas, a Seliberia, a Serpens, a Serpulina, a Serratia, a Shigella, a Simonsiella, a Sinorhizobium, a Sphaerotilus, a Sphingobacterium, a Spirillum, a Spirochaeta, a Spirosoma, a Spirulina, a Sporocytophaga, a Sporomusa, a Stella, a Stigmatalla, a Streptobacillus, a Succinimonas, a Succinivibrio, a Sulfobacillus, a Synechococcus, a Synechocystis, a Syntrophobacter, a Syntrophococcus, a Syntrophomonas, a Tatumella, a Taylorella, a Thermochromatium, a Thermodesulfobacterium, a Thermoleophilum, a Thermomicrobium, a Thermonema, a Thermosipho, a Thermotoga, a Thermus, a Thiobacillus, a Thiocapsa, a Thiococcus, a Thiocystis, a Thiodictyon, a Thiohalocapsa, a Thiolamprovum, a Thiomicrospira, a Thiorhodovibrio, a Thiothrix, a Tissierella, a Tolypothrix, a Treponema, a Vampirovibrio, a Variovorax, a Veillonella, a Verrucomicrobium, a Vibrio, a Vitreoscilla, a Weeksella, a Wolinella, a Xanthobacter, a Xanthomonas, a Xenococcus, a Xenorhabdus, a Xylella, a Xylophilus, a Yersinia, a Yokenella, a Zobellia, a Zoogloea, a Zymomonas, a Zymophilus, or a combination thereof.

Additional examples of an Eubacteria comprises an Abiotrophia, an Acetitomaculum, an Acetohalobium, an Acetonema, an Achromobacter, an Acidimicrobium, an Acidithiobacillus, an Acidobacterium, an Acidocella, an Acrocarpospora, an Actinoalloteichus, an Actinobacillus, an Actinobaculum, an Actinocorallia, an Aequorivita, an Afipia, an Agreia, an Agrococcus, an Ahrensia, an Albibacter, an Albidovulum, an Alcanivorax, an Alicycliphilus, an Alicyclobacillus, an Alkalibacterium, an Alkaliimnicola, an Alkalispirillum, an Alkanindiges, an Aminobacterium, an Aminomonas, an Ammonifex, an Ammoniphilus, an Anaeroarcus, an Anaerobacter, an Anaerobaculum, an Anaerobranca, an Anaerococcus, an Anaerofilum, an Anaeromusa, an Anaerophaga, an Anaeroplasma, an Anaerosinus, an Anaerostipes, an Anaerovorax, an Aneurinibacillus, an Angiococcus, an Anoxybacillus, an Antarctobacter, an Aquabacter, an Aquabacterium, an Aquamicrobium, an Aquifex, an Arcobacter, an Arhodomonas, an Asanoa, an Atopobium, an Azoarcus, an Azorhizophilus, an Azospira, a Bacteriovorax, a Bartonella, a Beutenbergia, a Bilophila, a Blastococcus, a Blastomonas, a Bogoriella, a Bosea, a Brachymonas, a Brackiella, a Brenneria, a Brevibacillus, a Bulleidia, a Burkholderia, a Caenibacterium, a Caldicellulosiruptor, a Caldithrix, a Caloramator, a Caloranaerobacter, a Caminibacter, a Caminicella, a Carbophilus, a Carboxydibrachium, a Carboxydocella, a Carboxydothermus, a Catenococcus, a Catenuloplanes, a Cellulosimicrobium, a Chelatococcus, a Chlorobaculum, a Chryseobacterium, a Chtysiogenes, a Citricoccus, a Collinsella, a Colwellia, a Conexibacter, a Coprothermobacter, a Couchioplanes, a Crossiella, a Ctyobacterium, a Cryptosporangium, a Dechloromonas, a Deferribacter, a Defluvibacter, a Dehalobacter, a Delftia, a Demetria, a Dendrosporobacter, a Denitrovibrio, a Dermacoccus, a Desemzia, a Desulfacinum, a Desulfitobacterium, a Desulfobacca, a Desulfobacula, a Desulfocapsa, a Desulfocella, a Desulfofaba, a Desulfofrigus, a Desulfofustis, a Desulfohalobium, a Desulfomusa, a Desulfonatronovibrio, a Desulfonatronum, a Desulfonauticus, a Desulfonispora, a Desulforegula, a Desulforhabdus, a Desulforhopalus, a Desulfospira, a Desulfosporosinus, a Desulfotalea, a Desulfotignum, a Desulfovirga, a Desulfurobacterium, a Desulfuromusa, a Dethiosulfovibrio, a Devosia, a Dialister, a Diaphorobacter, a Dichelobacter, a Dictyoglomus, a Dietzia, a Dolosicoccus, a Dorea, an Eggerthella, an Empedobacter, an Enhygromyxa, an Eremococcus, a Ferrimonas, a Filifactor, a Filobacillus, a Finegoldia, a Flexistipes, a Formivibrio, a Friedmanniella, a Frigoribacterium, a Fulvimonas, a Fusibacter, a Gallicola, a Garciella, a Gelidibacter, a Gelria, a Gemmatimonas, a Gemmobacter, a Geobacillus, a Geobacter, a Georgenia, a Geothrix, a Geovibrio, a Glaciecola, a Gluconacetobacter, a Gracilibacillus, a Granulicatella, a Grimontia, a Halanaerobacter, a Halanaerobium, a Haliangium, a Halobacillus, a Halocella, a Halonatronum, a Halothermothrix, a Halothiobacillus, a Helcococcus, a Heliophilum, a Heliorestis, a Herbidospora, a Hippea, a Holdemania, a Holophaga, a Hydrogenobacter, a Hydrogenobaculum, a Hydrogenophilus, a Hydrogenothermus, a Hydrogenovibrio, a Hymenobacter, an Ignavigranum, an Iodobacter, an Isobaculum, a Janibacter, a Kineococcus, a Kineosphaera, a Kitasatosporia, a Knoellia, a Kocuria, a Kozakia, a Kribbella, a Kutzneria, a Kytococcus, a Lachnobacterium, a Laribacter, a Lautropia, a Lechevalieria, a Leifsonia, a Leisingera, a Lentzea, a Leucobacter, a Limnobacter, a Listonella, a Lonepinella, a Luteimonas, a Luteococcus, a Macrococcus, a Macromonas, a Magnetospirillum, a Mannheimia, a Maricaulis, a Marinibacillus, a Marinitoga, a Marinobacterium, a Marinospirillum, a Marmoricola, a Meiothermus, a Methylocapsa, a Methylopila, a Methylosarcina, a Microbulbifer, a Microlunatus, a Micromonas, a Microsphaera, a Microvirgula, a Modestobacter, a Mogibacterium, a Moorella, a MoritaIla, a Muricauda, a Mycetocola, a Mycoplana, a Myroides, a Natroniella, a Natronincola, a Nautilia, a Nesterenkonia, a Nonomuraea, a Novosphingobium, an Oceanimonas, an Oceanobacillus, an Oceanobacter, an Octadecabacter, an Oenococcus, an Oleiphilus, an Oligotropha, an Olsenella, an Opitutus, an Orenia, an Omithinicoccus, an Omithinimicrobium, an Oxalicibacterium, an Oxalophagus, an Oxobacter, a Paenibacillus, a Pandoraea, a Papillibacter, a Paralactobacillus, a Paraliobacillus, a Parascardovia, a Paucimonas, a Pectobacterium, a Pelczaria, a Pelospora, a Pelotomaculum, a Peptoniphilus, a Petrotoga, a Phascolarctobacterium, a Phocoenobacter, a Photorhabdus, a Pigmentiphaga, a Planomicrobium, a Planotetraspora, a Plantibacter, a Plesiocystis, a Polaribacter, a Prauserella, a Propioniferax, a Propionimicrobium, a Propionispora, a Propionivibrio, a Pseudaminobacter, a Pseudoalteromonas, a Pseudobutyrivibrio, a Pseudoramibacter, a Pseudorhodobacter, a Pseudospirillum, a Pseudoxanthomonas, a Psychroflexus, a Psychromonas, a Psychroserpens, a Ralstonia, a Ramlibacter, a Raoultella, a Rathayibacter, a Rhodothermus, a Roseateles, a Roseburia, a Roseiflexus, a Roseinatronobacter, a Roseospirillum, a Roseovarius, a Rubritepida, a Ruegeria, a Sagittula, a Salana, a Salegentibacter, a Salinibacter, a Salinivibrio, a Sanguibacter, a Scardovia, a Schineria, a Schwartzia, a Sedimentibacter, a Shewanella, a Shuttleworthia, a Silicibacter, a Skermania, a Slackia, a Sphingobium, a Sphingomonas, a Sphingopyxis, a Spirilliplanes, a Sporanaerobacter, a Sporobacter, a Sporobacterium, a Sporotomaculum, a Staleya, a Stappia, a Starkeya, a Stenotrophomonas, a Sterolibacterium, a Streptacidiphilus, a Streptomonospora, a Subtercola, a Succiniclasticum, a Succinispira, a Sulfitobacter, a Sulfurospirillum, a Sutterella, a Suttonella, a Syntrophobotulus, a Syntrophothermus, a Syntrophus, a Telluria, a Tenacibaculum, a Tepidibacter, a Tepidimonas, a Tepidiphilus, a Terasakiella, a Terracoccus, a Tessaracoccus, a Tetragenococcus, a Tetrasphaera, a Thalassomonas, a Thauera, a Thermaerobacter, a Thermanaeromonas, a Thermanaerovibrio, a Thermicanus, a Thermithiobacillus, a Thermoanaerobacterium, a Thermobifida, a Thermobispora, a Thermobrachium, a Thermocrinis, a Thermocrispum, a Thermodesulforhabdus, a Thermodesulfovibrio, a Thermohydrogenium, a Thermomonas, a Thermosyntropha, a Thermoterrabacterium, a Thermovenabulum, a Thermovibrio, a Thialkalimicrobium, a Thialkalivibrio, a Thioalkalivibrio, a Thiobaca, a Thiomonas, a Tindallia, a Tolumonas, a Turicella, a Turicibacter, an Ureibacillus, a Verrucosispora, a Victivallis, a Virgibacillus, a Vogesella, a Weissella, a Williamsia, a Xenophilus, a Zavarzinia, a Zooshikella, a Zymobacter, or a combination thereof.

iii. Fungi

Organisms of the eukaryotic Fungi Kingdom (“fungi,” fungus”) include organisms commonly referred to as a molds, morels, mildews, mushrooms, puffballs, rusts, smuts, truffles, and yeasts. A fungal organism typically comprises multicellular filaments that grow into a food supply (e.g., a carbon based polymer), but may become unicellular spore(s) in nutrient poor conditions. “Mold” may be used herein as a synonym for fungi, where the context permits, especially when referring to indoor contaminants. However, the term “mold” also, and more specifically, denotes certain types of fungi. For example, the plasmodial slime molds, the cellular slime molds, water molds, and the everyday common mold. True molds refer to filamentous fungi comprising the mycelium, specialized, spore-bearing structures called conidiophores, and conidia (“spores”). “Mildew” is another common name for certain fungi, including a powdery mildew and a downy mildew. “Yeasts” are unicellular members of the fungus family. For the purposes of the present disclosure, where any of the terms fungus, a mold, a morel, a mildew, a mushroom, a puffball, a rust, a smut, a truffle, and/or a yeast is used, the others are implied where the context permits.

A fungi cell wall typically comprises a beta-1,4-linked homopolymers of N-acetylglucosamine (“chitin”) and a glucan. The glucan is usually an alpha-glucan, such as a polymer comprising an alpha-1,3- and alpha-1,6-linkage (Griffin, 1993). Some Ascomycota species (e.g., Ophiostomataceae) comprise a cell wall comprising a cellulose. Certain species of Chytridiomycota (e.g., Coelomomycetales) do not possess a cell wall (Alexopoulos et al., 1996). Examples of a fungi genus includes an Aciculoconidium, an Agaricostilbum, an Ambrosiozyma, an Arxiozyma, an Arxula, an Ascoidea, a Babjevia, a Bensingtonia, a Blastobotrys, a Botiyozyma, a Bullera, a Bulleromyces, a Candida, a Cephaloascus, a Chionosphaera, a Citeromyces, a Clavispora, a Cryptococcus, a Cystofilobasidium, a Debaiyomyces, a Dekkera, a Dipodascopsis, a Dipodascus, an Endomyces, an Eremothecium, an Erythrobasidium, a Fellomyces, a Filobasidiella, a Filobasidium, a Galactomyces, a Geotrichum, a Hanseniaspora, a Hyalodendron, an Issatchenkia, an Itersonilia, a Kloeckera, a Kluyveromyces, a Kockovaella, a Kurtzmanomyces, a Leucosporidium, a Lipomyces, a Lodderomyces, a Malassezia, a Metschnikowia, a Moniliella, a Mrakia, a Myxozyma, a Nadsonia, an Oosporidium, a Pachysolen, a Phaffia, a Pichia, a Protomyces, a Pseudozyma, a Reniforma, a Rhodosporidium, a Rhodotorula, a Saccaromycopsis, a Saccharomyces, a Saccharomycodes, a Saitoella, a Saturnispora, a Schizoblastosporion, a Schizosaccharomyces, a Sporidiobolus, a Sporobolomyces, a Sporopachydermia, a Stephanoascus, a Sterigmatomyces, a Sterigmatosporidium, a Sympodiomyces, a Sympodiomycopsis, a Taphrina, a Tilletiaria, a Tilletiopsis, a Torulaspora, a Trichosporon, a Trichosporonoides, a Trigonopsis, a Tsuchiyaea, a Wickerhamia, a Wickerhamiella, a Williopsis, a Xanthophyllomyces, a Yarrowia, a Zygoascus, a Zygosaccharomyces, a Zygozyma, or a combination thereof.

Examples of a fungal genus sometimes found in a building having excess indoor moisture comprises a Stachybotrys (e.g., a Stachybotrys chartarum), which is commonly found in nature growing on a cellulose-rich plant material and/or a water-damaged building material, such as ceiling tiles, wallpaper, sheet-rock and cellulose resin wallboard (e.g., a fiberboard). Depending on the particular conditions of temperature, pH and humidity in which the mold is growing, a Stachybotrys may produce mycotoxins, compounds that have toxic properties. Other examples of a common fungi that can grow in residential and commercial buildings comprise an Aspergillus species (sp.)., a Penicillium sp., a Fusarium sp., an Alternaria dianthicola, an Aureobasidium pullulans (a.k.a. a Pullularia pullulans), a Phoma pigmentivora and/or a Cladosporium sp. A proteinaceous composition (e.g., a peptide composition) may be selected to treat an infestation, prevent infestation, inhibit growth, and/or kill, a particular species of a cell such as a fungus and/or for a broad spectrum antifungal activity.

iv. Protista

Organisms of the Kingdom Protista (“protists”) refer to a heterogenous set of eukaryotic unicellular, oligocellular and/or multicellular organisms that may not have been classified as belonging to the other eukaryotic Kingdoms, though they typically have features related to the Plant Kingdom (e.g., an algae, which generally are photosynthetic), the Fungi Kingdom (e.g., an Oomycota) and/or the Animal Kingdom (e.g., a protozoa). Organisms of certain Protista Phyla, particularly those organisms commonly known as “algae,” comprise a cell wall, silica based shell and/or exoskeleton (e.g., a test, a frustule), or other durable material at the cell-external environment interface.

Examples of a Protista comprises an Acetabularia, an Achnanthes, an Amphidinium, an Ankistrodesmus, an Anophryoides, an Aphanomyces, an Astasia, an Asterionella, a Blepharisma, a Botrydiopsis, a Botrydium, a Botryococcus, a Bracteacoccus, a Brevilegnia, a Bulbochaete, a Caenomorpha, a Cephaleuros, a Ceratium, a Chaetoceros, a Chaetophora, a Characiosiphon, a Chlamydomonas, a Chlorella, a Chloridella, a Chlorobotrys, a Chlorococcum, a Chromulina, a Chroodactylon, a Chrysamoeba, a Chtysocapsa, a Cladophora, a Closterium, a Cocconeis, a Coelastrum, a Cohnilembus, a Colacium, a Coleps, a Colpidium, a Colpoda, a Cosmarium, a Cryptoglena, a Cyclidium, a Cyclotella, a Cylindrocystis, a Derbesia, a Dexiostoma, a Dictyosphaerium, a Dictyuchus, a Didinium, a Dinobryon, a Distigma, a Draparnaldia, a Dunaliella, a Dysmorphococcus, an Enteromorpha, an Entosiphon, an Eudorina, an Euglena, an Euplotes, an Eustigmatos, a Flintiella, a Fragilaria, a Fritschiella, a Glaucoma, a Gonium, a Gonyaulax, a Gymnodinium, a Gyropaigne, a Haematococcus, a Halophytophthora, a Heterosigma, a Hyalotheca, a Hydrodictyon, a Khawkinea, a Lagenidium, a Leptolegnia, a Mallomonas, a Mantoniella, a Melosira, a Menoidium, a Mesanophrys, a Mesotaenium, a Metopus, a Micrasterias, a Microspora, a Microthamnion, a Mischococcus, a Monodopsis, a Mougeotia, a Nannochloropsis, a Navicula, a Nephroselmis, a Nitzschia, an Ochromonas, an Oedogonium, an Ophiocytium, an Opisthonecta, an Oxyrrhis, a Pandorina, a Paramecium, a Paranophrys, a Paraphysomonas, a Pamidium, a Pediastrum, a Peranema, a Peridinium, a Peronophythora, a Petalomonas, a Phacus, a Pithophora, a Plagiopyla, a Plasmopara, a Platyophtya, a Plectospira, a Pleodorina, a Pleurochloris, a Pleurococcus, a Pleurotaenium, a Ploeotia, a Polyedriella, a Porphyridium, a Prorocentrum, a Prototheca, a Pseudocharaciopsis, a Pseudocohnilembus, a Pyramimonas, a Pythiopsis, a Pythium, a Rhabdomonas, a Rhizochromulina, a Rhizoclonium, a RhodeIla, a Rhodosorus, a Rhynchopus, a Saprolegnia, a Scenedesmus, a Scytomonas, a Selenastrum, a Skeletonema, a Spathidium, a Sphaerocystis, a Spirogyra, a Spirostomum, a Spondylosium, a Staurastrum, a Stauroneis, a Stentor, a Stephanodiscus, a Stephanosphaera, a Stichococcus, a Stigeoclonium, a Synedra, a Synura, a Tetracystis, a Tetraedron, a Tetrahymena, a Tetraselmis, a Thalassiosira, a Thaumatomastix, a Thraustotheca, a Trachelomonas, a Trebouxia, a Trentepohlia, a Tribonema, a Trimyema, an Ulothrix, an Uronema, a Vaucheria, a Vischeria, a Volvox, a Vorticella, a Xanthidium, a Zygnema, or a combination thereof.

A diatom refers to a unicellular algae that possess a cell wall comprising silicon. Examples of a diatom include organisms of the phyla Chrysophyta and/or Bacillariphyta. A Chrysophyta (“golden algae,” “golden-brown algae”) typically comprises a freshwater diatom. Examples of a Chrysophyta includes a Chlorobottys, a Chromulina, a Chrysamoeba, a Chtysocapsa, a Dinobryon, an Eustigmatos, a Heterosigma, a Mallomonas, a Monodopsis, a Nannochloropsis, an Ochromonas, a Paraphysomonas, a Pleurochloris, a Polyedriella, a Pseudocharaciopsis, a Rhizochromulina, a Synura, a Thaumatomastix, a Vischeria, or a combination thereof. A Bacillariphyta typically comprises a marine diatom. Examples of a Bacillariphyta includes an Achnanthes, an Asterionella, a Chaetoceros, a Cocconeis, a Cyclotella, a Fragilaria, a Melosira, a Navicula, a Nitzschia, a Skeletonema, a Stauroneis, a Stephanodiscus, a Synedra, a Thalassiosira, or a combination thereof.

A Xanthophyta (“yellow-green algae”) is typically yellowish-green in color, with examples including a Botrydiopsis, a Botrydium, a Botryococcus, a Chloridella, a Mischococcus, an Ophiocytium, a Tribonema, a Vaucheria, or a combination thereof.

An Euglenophyta (“euglenoids”) generally is unicellular, aquatic algae and comprises a pellicle, which comprises an outer membrane reinforced by proteins, rather than a cell wall. Examples of an Euglenophyta include an Astasia, a Colacium, a Cryptoglena, a Distigma, an Entosiphon, an Euglena, a Gyropaigne, a Khawkinea, a Menoidium, a Pamidium, a Peranema, a Petalomonas, a Phacus, a Ploeotia, a Rhabdomonas, a Rhynchopus, a Scytomonas, a Trachelomonas, or a combination thereof.

A Chlorophyta (“green algae”) typically forms unicellular to oligocellular cluster(s), and comprises a cell wall comprising a cellulose. Examples of a Chlorophyta include a Volvox, a Chloralla, a Pleurococcus, a Spirogyra, a Chlamydomonas, a Gonium, a Mantoniella, a Nephroselmis, a Pyramimonas, a Tetraselmis, an Ulothrix, an Enteromorpha, a Cephaleuros, a Cladophora, a Pithophora, a Rhizoclonium, a Derbesia, an Acetabularia, a Chloralla, a Microthamnion, a Prototheca, a Stichococcus, a Trebouxia, an Ankistrodesmus, a Bracteacoccus, a Bulbochaete, a Chaetophora, a Characiosiphon, a Chlamydomonas, a Chlorococcum, a Coelastrum, a Dictyosphaerium, a Draparnaldia, a Dunaliella, a Dysmorphococcus, an Eudorina, a Fritschiella, a Gonium, a Haematococcus, a Hydrodictyon, an Oedogonium, a Microspora, a Pandorina, a Pediastrum, a Pleodorina, a Scenedesmus, a Selenastrum, a Sphaerocystis, a Stephanosphaera, a Stigeoclonium, a Tetracystis, a Tetraedron, a Trentepohlia, an Uronema, a Volvox, a Closterium, a Cosmarium, a Cylindrocystis, a Hyalotheca, a Mesotaenium, a Micrasterias, a Mougeotia, a Pleurotaenium, a Spirogyra, a Spondylosium, a Staurastrum, a Xanthidium, a Zygnema, or a combination thereof.

A Rhodophyta (“red algae”) generally is multicellular and comprises a cell wall comprising a sulfated polysaccharide, such as, for example, an agar, a carrageenan, a cellulose, or a combination thereof.

Examples of a Rhodophyta genera that are typically unicellular include a Chroodactylon, a Flintiella, a Porphyridium, a Rhodella, a Rhodosorus, or a combination thereof.

A Pyrrophyta (“fire algae,” “dinoflagellate”) generally is a unicellular marine organism possessing a cell wall comprising cellulose. A Pyrrophyta typically is red, and examples include a dinoflagellate genera such as an Amphidinium, a Ceratium, a Gonyaulax, a Gymnodinium, an Oxyrrhis, a Peridinium, a Prorocentrum, or a combination thereof.

A Ciliophora (“ciliate”) generally is unicellular and comprises a pellicle. Examples of a Ciliophora includes an Anophryoides, a Blepharisma, a Caenomorpha, a Cohnilembus, a Coleps, a Colpidium, a Colpoda, a Cyclidium, a Dexiostoma, a Didinium, an Euplotes, a Glaucoma, a Mesanophrys, a Metopus, an Opisthonecta, a Paramecium, a Paranophrys, a Plagiopyla, a Platyophrya, a Pseudocohnilembus, a Spathidium, a Spirostomum, a Stentor, a Tetrahymena, a Trimyema, an Uronema, a Vorticella, or a combination thereof.

An Oomycota (“oomycete,” “water mold”) is a fungi-like organism, and is often listed in the fungal sections of biological culture collections. An Oomycota is typically unicellular but differ from a fungi by possessing a cell wall that comprises a cellulose and/or a glycan. Examples of an Oomycota an Aphanomyces, a Brevilegnia, a Dictyuchus, a Halophytophthora, a Lagenidium, a Leptolegnia, a Peronophythora, a Plasmopara, a Plectospira, a Pythiopsis, a Pythium, a Saprolegnia, a Thraustotheca, or a combination thereof.

v. Viruses

Examples of a virus (e.g., an enveloped virus) that may be a target of an antibiological agent includes a DNA virus such as a Herpesviridae (“herpesviruses”), a Poxyiridae (“poxviruse”), and/or a Baculoviridae (“baculooviruses”); an RNA virus such as a Flaviviridae (“flavivirus”), a Togaviridae (“togavirus”), a Coronaviridae (“coronavirus”; e.g., Severe Acute Respiratory Syndrome-“SARS”), a Deltaviridae (“deltavirus”; e.g., Hepatitis D), an Orthomyxoviridae (“orthomyxovirus”), a Paramyxoviridae (“paramyxovirus”), a Rhabdoviridae (“rhabdovirus”), a Bunyaviridae (“bunyavirus”), a Filoviridae (“filovirus”), and/or a Reoviridae (“Reovirus”); a retrovirus such as a Retroviridae (“retroviruses”), and/or a Hepadnaviridae (“hepadnavirus”); or a combination thereof.

b. Cellular Components

In many embodiments, a component of a cell wall, a viral proteinaceous molecule, and/or a cellular membrane may comprise a target of an antibiological agent; may comprise a component of a cell-based particulate material, or a combination thereof. Examples of such a cell wall, a viral proteinaceous molecule, and/or a cellular membrane component includes a peptidoglycan, a pseudopeptidoglycan, a teichoic acid, a teichuronic acid, a cellulose, a neutral polysaccharide, a chitin, a mannin, a glucan, a proteinaceous molecule, a lipid (e.g., a phospholipid), or a combination thereof. These cell and/or viral component(s) may function as an antibiological agent's target such as an antibiological enzyme substrate and/or a ligand for a proteinaceous molecule's binding interaction (e.g., an antibiological peptide binding); as well as possibly function as a component(s) of a cell-based particulate material.

i. Peptidoqlycans and Pseudopeptidoqlycans

An Eubacteria cell wall typically comprise a peptidoglycan (“mucopeptide,” “murein”), as well as a glycoprotein, a protein, a polysaccharide, a lipid, or a combination thereof. Peptidoglycan generally comprises alternating monomers of the amino-sugars N-acetylglucosamine and N-acetylmuramic acid. The N-acetylmuramic acid monomers often further comprise a tetra-peptide of the sequence L-alanine-D-glutamic acid-L-diamino acid-D-alanine covalently bonded to the muramic acid. The attached tetrapeptides of peptidoglycan participate in cross-linking a plurality of polymers to contribute to the cell wall structure. Depending on the species, the tetrapeptides may form the cross-linkages by direct covalent bonds, and/or one or more amino acids may form the cross-linking bonds between the tetrapeptides. A biomolecule used in many embodiments may comprise a peptidoglycan for conferring particulate nature and durability to various cell-based particulate materials, given the general ease of growth of Eubacteria. Archaea do not possess peptidoglycan, but many Archaea may comprise a pseudopeptidoglycan, which comprises N-acetyltalosaminuronic acid, instead of N-acetylmuramic in peptidoglycan.

ii. Teichoic Acids and Teichuronic Acids

A cell wall, particularly of Gram-positive Eubacteria, may comprise up to 50% teichoic acid. Teichoic acid comprises an acidic polymer comprising monomers of a phosphate and a glycerol; a phosphate and a ribitol; and/or a N-acetylglucosamine and a glycerol. A sugar (e.g., glucose) and/or an amino acid (e.g., D-alanine) usually attaches to the glycerol and/or the ribitol of a teichoic acid. In addition to direct association with and/or integration into a cell wall, a teichoic acid may be associated with a phospholipid bilayer adjacent to a cell wall. Often, a teichoic acid may be covalently bonded to a glycolipid of a cell membrane, and may be known as a “lipoteichoic acid.” Teichic acids are common in a Staphylococcus, a Micrococcus, a Bacillus, and/or a Lactobacillus genera.

A cell wall of certain species of Gram-positive Eubacteria may comprise teichuronic acid. Teichuronic acid comprises a polymer comprising a N-acetylglucosamine and a glucuronic acid; and/or a glucose and an amino-mannuronic acid. However, acidic conditions may damage this cell wall component, as an uronic acid such as a glucuronic acid, and particularly an amino-mannuronic acid, may be hydrolyzed in an acid. Exposure to acid during processing and/or in a material formulation may reduce this component from a cell based particulate matter.

iii. Neutral Polysaccharides

A cell wall, particularly of a Gram-positive Eubacteria, may comprise a neutral polysaccharide, other than those described for a peptidoglycan, a teichoic acid, a cellulose, and/or a lipopolysacharide. As used herein, a “neutral polysaccharide” comprises a polymer comprising a majority of neutral sugars, wherein the neutral sugar typically comprises a hexose, a pentose, and/or an amino sugar thereof. Examples of a neutral sugar found in a neutral polysaccharide include an arabinose, a galactose, a 3-O-methyl-D-galactose, a mannose, a xylose, a rhamnose, a glucose, a fructose, or a combination thereof. Examples of an amino sugar found in a neutral polysaccharide include a glucosamine, a galactosamine, or a combination thereof.

iv. Proteinaceous Molecules

A cell wall and/or a virus may comprise a proteinaceous molecule, such as, for example, a polypeptide, a peptide, a protein. In some aspects, such a proteinaceous material may dominate the structural integrity that confers particulate material durability to a virus and/or a cell comprising a pellicle. Additionally, peptide linkage(s) are common throughout a peptidoglycan and/or a pseudopeptidoglycan.

v. Lipids

A cell wall may comprise a lipid, other than those described for a peptidoglycan, a teichoic acid, and/or a lipopolysacharide. Typically, a cell comprises various lipid biomolecules, which generally comprise fatty acids. In embodiments wherein a processing step comprises contacting the cell with a non-aqueous solvent, lipids may be removed from a cell and/or a cell wall. However, in embodiments wherein such a processing step does not occur, the lipid components of a cell and/or a cell wall remaining in the particulate matter may affect a material formulation's reactions wherein lipid (e.g., a fatty acid double bond) cross-linking activity contributes to preparation/processing/use (e.g., film-formation of a coating). Lipids of particular relevance for such a potential cross-linking reaction include those of the outer membrane, which comprise a fatty acid, the cell wall, or a combination thereof.

For example, Gram-negative cells comprise a phospholipid bilayer often referred to as the “outer cell membrane” that surrounds the cell wall. A “phospholipid bilayer” comprises two layers of phospholipid molecules, wherein the fatty acids components of each layer's phospholipids contact each other, thereby creating a hydrophobic inner region, and the head groups of each layer's phospholipids, which are generally hydrophilic, contact the external environment. Examples of a phospholipid include a glycerophospholipid, which comprises two fatty acids and one hydrophilic moiety called a “head group” covalently connected to a trihydroxyl alcohol glycerol. Non-limiting examples of a head group include a choline, an ethanolamine, a serine, an inositol, an additional glycerol, or a combination thereof. Additionally, a phospholipid bilayer generally comprises a plurality of peptides and/or polypeptides with hydrophobic regions that are retained in the phospholipid bilayer's hydrophobic inner region. The cell wall peptidoglycan may be linked to the phospholipid membrane by a periplasmic space lipoprotein.

Gram-positive Eubacteria cell walls generally comprise about 0% to about 2% lipid. However, certain categories of Gram-positive Eubacteria may comprise up to about 50% or more lipid content as part of the cell wall. Such Eubacteria include different species of Gordonia, Mycobacterium, Nocardia, and Rhodococcus. Additionally, the lipids of such Eubacteria generally comprise a branched chain fatty acid, particularly mycolic acids (Barry, C. E. et al., Prog Lipid Res 37:143, 1998). A mycolic acid may be covalently bound and/or loosely associated with a cell wall sugar. The type of Eubacteria may be sometimes used to identify the carbon-backbone length of a mycolic acid. For example, an eumycolic acid may be isolated from a Mycobacterium, and generally comprises about 60 to about 90 carbon atoms. A corynomycolic acid isolated from a Corynobacterium generally comprises 22 to 36 carbons. A nocardomycoic acid isolated from a Nocardia generally comprises 44 to 60 carbons. A mycolic acid generally comprises a fatty acid branch (“alpha branch”) and an aldehyde (“meromycolate branch”). A mycolic acid may further comprise a carbon double bond, an epoxy ester moiety, a cyclopropane ring moiety, a keto moiety, a methoxy moiety, or a combination thereof, generally located on a meromycolate branch. A mycolic acid may comprise an α-mycolic acid, a methoxymycolic acid, a ketomycolic acid, an epoxymycolic acid, a wax ester, or a combination thereof. A α-mycolic acid comprises a cis or trans carbon double bond and/or a cyclopropane, and may further comprise a methyl branch adjacent to such a moiety. A methoxymycolic acid comprises a methoxy moiety and a double bond or a cyclopropane. A ketomycolic acid comprises α-methyl-branched ketone. An epoxymycolic acid comprises an α-methyl-branch epoxide. A wax ester comprises an internal ester group and a carbon double bond or a cyclopropane ring.

In certain facets, a cell lipid may comprise a glycolipid, which refers to a glycan covalently attached to a lipid. Non-limiting examples of a glycolipid include a dolichyl phosphoryl glycan, a pyrophosphoryl glycan, an undecaprenyl phosphoryl glycan, a pryophosphoryl glycan, a retinyl phosphoryl glycan, a glycosphingolipid (e.g., a ceramide, a galactosphingolipid, a glucosphingolipid including a ganlioside), a glycoglycerolipid (e.g., a monogalactosyldiacylglycerol), a steroidal glycoside (e.g., ouabain, digoxin, digitonin), a glycosylated phosphoinositide (e.g., a GPI anchor, a lipophosphoglycan, a lipopeptidophosphoglycan, a glycoinositol phospholipid), or a combination thereof.

The phospholipid bilayers of Archaea are biochemically distinct from the lipids described above, as they comprise branched hydrocarbon chains attached to glycerol by ether linkages instead of fatty acids attached to glycerol by ester linkages.

vi. Celluloses

A cell wall of organisms, primarily of the Kingdom Planta, comprises cellulose. Cellulose comprises a polysaccharide polymer (e.g., a linear polymer) typically hundreds to thousands of glucose monomer units long, and commonly functions as a structural component of the primary cell wall of green plants and many forms of algae. In addition, some bacteria form a biofilm by secreting cellulose, and some Ascomycota fungal species (e.g., an Ophiostomataceae) comprise cell walls made of cellulose.

vii. Chitins

Fungi cells and spore wall components typically include beta-1,4-linked homopolymers of a N-acetylglucosamine (“chitin”) and a glucan. A chitin is similar to a cellulose, though an acetylamine moiety (N-acetylglucosamine) substitutes for a hydroxyl moiety on the glucose monomer(s). The relative increase in hydrogen bonding between chitin polymer chains enhances the strength of a chitin-polymer matrix. The glucan usually comprises an alpha-glucan, such as a polymer comprising an alpha-1,3- and an alpha-1,6-linkage (Griffin, 1993).

viii. Agaroses

Agarose and porphyran comprise polysaccharide polymers, and are components of some algae (e.g., red algae).

ix. Mannins and Glucans

A fungal cell wall (e.g., a yeast cell wall) may comprise an oligo-mannan, a helical β(1-3)-D-glucan, and/or a β(1-3)-D-glucan, well as a chitin, lipid(s) and/or protein(s). A linkage (e.g., a β(1-4)-linkage) may occur, for example between the nonreducing ends of a glucan and a glycoprotein; and the reducing ends of chitin (Kollar, R., et al., 1995; Kapteyn, J. C., et al., 1996).

### D. MULTIFUNCTIONAL ENZYMES

In some embodiments, a biomolecule such as an enzyme may possess one or more secondary characteristics, functions and/or activities (e.g., a binding activity, a catalytic activity) in addition to the characteristic, the function and/or the activity of its classification (e.g., EC classification) and/or characterization. In some aspects, such a multifunctional enzyme may be selected for use based on the secondary activity over the primary activity of its classification. In some embodiments, an enzyme may be selected for both its primary activity and a secondary activity. For example, some carboxylesterases (EC 3.1.1.1) have demonstrated this binding and/or catalytic property against a soman, a diazinon and/or a malathion (e.g., Rattus norvegicus ES4 and ES10; enzymes from a Plodia interpunctella, a Chrysomya putoria, a Lucilia cuprina, a Musca domestica, a Myzus persicae, and/or a Homo sapiens liver cell). Often an organophosphorus compound acts as an inhibitor of the carboxylesterase, though hydrolysis occurs in some instances [In “Esterases, Lipases, and Phospholipases from Structure to Clinical Significance.” (Mackness, M. I. and Clerc, M., Eds.), pp. 91-98, 1994]. Many genes in an organism (e.g., an eukaryatic organism) have multiple alleles which comprise a variant nucleotide and/or an expressed protein sequence for a particular gene. In particular, an allele of a carboxylesterase gene possessing an organophosphate hydrolase (EC 3.1.8.1) activity may be responsible for OP compound resistance. Examples of such a carboxylesterase gene include an allele isolated from Lucilia cuprina (Genbank accession no. U56636; Entrez databank no. AAB67728), Musca domestica (Genbank accession no. AF133341; Entrez databank no. AAD29685), or a combination thereof (Claudianos, C. et al., 1999; Campbell, P. M. et al., 1998; Newcomb, R. D. et al., 1997). In an additional example, depending on the application and an enzymatic/binding activity of a carboxylesterase, such a multifunctional carboxylesterase may be selected for a lipolytic activity in one application, and selected for an organophosphorus compound binding and/or hydrolytic activity in a different application. Such a multifunctional carboxylesterase may be differentiated herein by the use of “carboxylesterase” when referring to an enzyme as a lipolytic enzyme, and a “carboxylase” when referring to an enzyme used for function as an organophosphorus compound binding/degrading enzyme.

In an additional example, a carboxylesterase and/or a carbamoyl lyase may be useful against a carbamate nerve agent, and are specifically contemplated for use in a biomolecular composition and/or a material formulation for use against such a carbamate nerve agent.

In a further example, a prolidase (“imidodipeptidase,” “proline dipeptidase,” “peptidase D,” “g-peptidase”), a PepQ and/or an aminopeptidase P gene and/or a gene product may possess, for example, an OPAA activity. OPAAs possess sequence and structural similarity to a human prolidase, an Escherichia coli aminopeptidase P and/or an Escherichia coli PepQ (Cheng, T.-C. et al., 1997; Cheng, T.-C. et al., 1996). A prolidase and/or a PepQ protein (E.C. 3.4.13.9) hydrolyze a C—N bond of a dipeptide with a prolyl residue at the carboxyl-terminus, and an OPAA may also be have prolidase activity. An aminopeptidase P (EC 3.4.11.9) hydrolyzes the C—N amino bond of a proline at the penultimate position from the amino terminus of an amino acid sequence. A partly purified human and/or a porcine prolidase demonstrated the ability to cleave DFP and G-type nerve agents (Cheng, T.-C. et. al., 1997). Examples of prolidase genes and gene products include a Mus musculus prolidase gene (GeneBank accession no. D82983; Entrez databank no. BAB11685); a Homo sapien prolidase gene (GeneBank accession no. J04605; Entrez databank AAA60064); a Lactobacillus helveticus prolidase (“PepQ”) gene (GeneBank accession no. AF012084; Entrez databank AAC24966); an Escherichia coli prolidase (“pepQ”) gene (GeneBank accession no. X54687; Entrez databank CAA38501); an Escherichia coli aminopeptidase P (“pepP”) gene (GeneBank accession no. D00398; Entrez databank BAA00299; Protein Data Bank entries 1A16, 1AZ9, 1JAW and 1M35); or a combination thereof (Ishii, T. et al., 1996; Endo, F. et al., 1989; Nakahigashi, K. and Inokuchi, H., 1990; Yoshimoto, T. et al., 1989).

In an additional example, certain cholinesterases (e.g., an acetyl cholinesterase) with OP degrading activity have been identified in insects resistant OP pesticides (see, for example, Baxter, G. D. et al., 1998; Baxter, G. D. et al., 2002; Rodrigo, L., et al., 1997, Vontas, J. G., et al., 2002; Walsh, S. B., et al., 2001; Zhu, K. Y., et al., 1995), and are contemplate for use.

### E. FUNCTIONAL EQUIVALENTS OF WILD-TYPE PROTEINACEOUS MOLECULES

It is possible to improve a proteinaceous molecule (e.g., an enzyme, an antibody, a receptor, a peptide, a polypeptide) with a defined amino acid sequence and/or length for one or more properties. An alteration in a property is possible because such molecules may be manipulated, for example, by chemical modification, including but not limited to, modifications described herein. As used herein “alter” or “alteration” may result in an increase or a decrease in the measured value for a particular property. Examples of a property, in the context of a proteinaceous molecule, includes, but is not limited to, a ligand binding property, a catalytic property, a stability property, a property related to environmental safety, a charge property, or a combination thereof. Examples of a catalytic property that may be altered include a kinetic parameter, such as Kₘ, a catalytic rate (k_{cat}) for a substrate, an enzyme's specificity for a substrate (k_{cat}/Kₘ), or a combination thereof. Examples of a stability property that may be altered include thermal stability, half-life of activity, stability after exposure to a weathering condition, or a combination thereof. Examples of a property related to environmental safety include an alteration in toxicity, antigenicity, bio-degradability, or a combination thereof. However, an alteration to increase an enzyme's catalytic rate for a substrate, an proteinaceous molecule's specificity and/or binding property(s) for a ligand, a proteinaceous molecule's thermal stability, a proteinaceous molecule's half-life of activity, and/or a proteinaceous molecule's stability after exposure to a weathering condition may be selected for some applications, while a decrease in toxicity and/or antigenicity for a proteinaceous molecule may be selected in additional applications. A proteinaceous molecule (e.g., an enzyme, an antibody, a receptor, a peptide, a polypeptide) comprising a chemical modification and/or a sequence modification that functions the same or similar (e.g., a modified enzyme of the same EC classification as the unmodified enzyme) comprises a “functional equivalent” to, and “in accordance” with, an un-modified proteinaceous molecule.

There may be a limit to the number of chemical modifications that may be made to a proteinaceous molecule (e.g., an enzyme, an antibody, a receptor, a peptide, a polypeptide) before a property may be undesirably altered. However, in light of the disclosures herein of assays for determining whether a composition possesses one or more properties, including, for example, an enzymatic activity, a stability property, a binding property, etc., using, but not limited to the assays described herein, to determine whether a given chemical modification to a proteinaceous molecule (e.g., an enzyme, an antibody, a receptor, a peptide, a polypeptide) produces a molecule that still possesses a suitable set of properties for use in a particular application. For example, a functional equivalent enzyme comprising a plurality of different chemical modifications may be produced.

A functional equivalent proteinaceous molecule comprising a structural analog and/or a sequence analog may possess an altered, an enhanced property and/or a reduced property, in comparison to the proteinaceous molecule upon which it is based. As used herein, a “structural analog” refers to one or more chemical modifications to the peptide backbone and/or non-side chain chemical moiety(s) of a proteinaceous molecule. In certain aspects, a subcomponent of an proteinaceous molecule such as an apo-enzyme, a prosthetic group, a co-factor, or a combination thereof, may be modified to produce a functional equivalent structural analog. In particular facets, such an proteinaceous molecule sub-component that does not comprise a proteinaceous molecule may be altered to produce a functional equivalent structural analog of an proteinaceous molecule when combined with the other sub-components. As used herein, a “sequence analog” refers to one or more chemical modifications to the side chain chemical moiety(s), also known herein as a “residue” of one or more amino acids that define a proteinaceous molecule's sequence. Often such a “sequence analog” comprises an amino acid substitution, which may be produced by recombinant expression of a nucleic acid comprising a genetic mutation to produce a mutation in the expressed amino acid sequence.

As used herein, an “amino acid” may comprise a common and/or an uncommon amino acid. The common amino acids include: alanine (Ala, A); arginine (Arg, R); aspartic acid (a.k.a. aspartate; Asp, D); asparagine (Asn, N); cysteine (Cys, C); glutamic acid (a.k.a. glutamate; Glu, E); glutamine (Gln, Q); glycine (Gly, G); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V). Common amino acids are often biologically produced in the biological synthesis of a peptide and/or a polypeptide. An uncommon amino acid refers to an analog of a common amino acid (e.g., a D isomer of an L-amino acid), as well as a synthetic amino acid whose side chain may be chemically unrelated to the side chains of the common amino acids (e.g., a norleucine). An amino acid may comprise a D-amino acid, an L-amino acid, and/or a cyclic (non-racemic) amino acid. A proteinaceous sequence (e.g., a peptide) may be constructed as retroinversopeptidomimetic of a proteinaceous sequence (e.g., a D-configuration, an L-configuration. The chemical structure of such amino acids (which term is used herein to include imino acids), regardless of stereoisomeric configuration, may be based upon that of the naturally-occurring (e.g., a common) amino acid: Various uncommon amino acids may be used, though general embodiments, an proteinaceous molecule may be biologically produced, and thus lack or possess relatively few uncommon amino acids prior to any subsequent non-mutation based chemical modifications. I

Thus, for example, a proteinaceous molecule (e.g., an antifungal peptide, an antibacterial peptide, an antifouling peptide) may comprise an amino acid such as a common amino acid, an uncommon amino acid, an L-amino acid, a D-amino acid, a cyclic (non-racemic) amino, or a combination thereof. In some embodiments, such a proteinaceous molecule may act rapidly and/or have reduced stability. In other embodiments, a D-amino acid may increase the stability of a proteinaceous molecule, such as making the proteinaceous molecule insensitive and/or less susceptible to an L-amino acid biodegradation pathway. In a specific example, an L-amino acid peptide may be stabilized by addition of a D-amino acid at one or both of the peptide termini. However, biochemical pathways are available which may degrade a proteinaceous molecule comprising a D-amino acid, and may reduce long-term environmental persistence of such a proteinaceous molecule.

The side chains of amino acids comprise one or more moiety(s) with specific chemical and physical properties. Certain side chains contribute to a ligand binding property, a catalytic property, a stability property, a property related to environmental safety, or a combination thereof. For example, cysteines may form covalent bonds between different parts of a contiguous amino acid sequence, and/or between non-contiguous amino acid sequences to confer enhanced stability to a secondary, tertiary and/or quaternary structure. In an additional example, the presence of hydrophobic or hydrophilic side chains exposed to the outer environment may alter the hydrophobicity or hydrophilicity of part of a proteinaceous sequence, such as in the case of a transmembrane domain embedded in a lipid layer of a membrane. In another example, hydrophilic side chains may be exposed to the environment surrounding a proteinaceous molecule, which may enhance the overall solubility of a proteinaceous molecule in a polar liquid, such as water and/or a liquid component of a material formulation. In a further example, various acidic, basic, hydrophobic, hydrophilic, and/or aromatic side chains present at or near a binding site of a proteinaceous structure may affect the affinity for a proteinaceous sequence for binding a ligand and/or a substrate, based on the covalent, ionic, Van der Waal forces, hydrogen bond, hydrophilic, hydrophobic, and/or aromatic interactions at a binding site. Such interactions by residues at or near an active site also contribute to a chemical reaction that occurs at the active site of an enzyme to produce enzymatic activity upon a substrate. As used herein, a residue may be “at or near” a residue and/or a group of residues when it is within about 15 Å, about 14 Å, about 13 Å, about 12 Å, about 11 Å, about 10 Å, about 9 Å, about 8 Å, about 7 Å, about 6 Å, about 5 Å, about 4 Å, about 3 Å, about 2 Å, and/or about 1 Å the residue or group of residues such as residues identified as contributing to the active site and/or the binding site of a proteinaceous molecule.

Identification of an amino acid whose chemical modification may likely change a property of a proteinaceous molecule may be accomplished using such methods as a chemical reaction, mutation, X-ray crystallography, nuclear magnetic resonance (“NMR”), computer based modeling, or a combination thereof. Selection of an amino acid on the basis of such information may then be used in the rational design of a mutant proteinaceous sequence that may possess an altered property. Alterations include those that alter a proteinaceous molecule's activity and/or function (e.g., binding activity, enzymatic activity, antimicrobial activity) to produce a functional equivalent of a proteinaceous moleculee.

For example, many residues of a proteinaceous molecule that contribute to the properties of a proteinaceous molecule comprise chemically reactive moiety(s). These residues are often susceptible to chemical reactions that may inhibit their ability to contribute to a property of the proteinaceous molecule. Thus, a chemical reaction may be used to identify one or more amino acids comprised within the proteinaceous molecule that may contribute to a property. The identified amino acids then may be subject to modifications such as amino acid substitutions to produce a functional equivalent. Examples of amino acids that may be so chemically reacted include Arg, which may be reacted with butanedione; Arg and/or Lys, which may be reacted with phenylglyoxal; Asp and/or Glu, which may be reacted with carbodiimide and HCl; Asp and/or Glu, which may be reacted with N-ethyl-5-phenylisoxazolium-3′-sulfonate (“Woodward's reagent K”); Asp and/or Glu, which may be reacted with 1,3-dicyclohexyl carbodiimide; Asp and/or Glu, which may be reacted with 1-ethyl-3-(3-dimethylaminopropyl)carbodiimide (“EDC”); Cys, which may be reacted with p-hydroxy mercuribenzoate; Cys, which may be reacted with dithiobisnitrobenzoate (“DTNB”); Cys, which may be reacted with iodoacetamide; H is, which may be reacted with diethylpyrocarbonate (“DEPC”); His, which may be reacted with diazobenzenesulfonic acid (“DBS”); His, which may be reacted with 3,7-bis(dimethylamino)phenothiazin-5-ium chloride (“methylene blue”); Lys, which may be reacted with dimethylsuberimidate; Lys and/or Arg, which may be reacted with 2,4-dinitrofluorobenzene; Lys and/or Arg, which may be reacted with trinitrobenzene sulfonic acid (“TNBS”); Trp, which may be reacted with 2-hydroxy-5-nitrobenzyl bromide 1-ethyl-3(3-dimethylaminopropyl); Trp, which may be reacted with 2-acetoxy-5-nitrobenzyl chloride; Trp, which may be reacted with N-bromosucinimide; Tyr, which may be reacted with N-acetylimidazole (“NAI”); or a combination thereof (Hartleib, J. and Ruterjans, H., 2001b; Josse, D. et al., 1999; Josse, D. et al., 2001).

A variety of modifications of the art can be made to a proteinaceous molecule (e.g., a peptide), particularly a modification that may confer, retain, and/or alter a property (e.g., an antibiological activity). For example, some modifications may be used to increase the intrinsic antifungal potency of a peptide. In another example, though a modification may reduce an antibiological activity of a proteinaceous molecule, such a reduction may still produce a proteinaceous molecule with suitable antibiological activity. Other modifications may facilitate handling of a peptide. Other modifications may alter a binding property. A proteinaceous molecule's (e.g., a peptide) functional moiety that may typically be modified include a hydroxyl, an amino, a guanidinium, a carboxyl, an amide, a phenol, an imidazol ring(s), and/or a sulfhydryl. Typical reactions of these moieties include, for example, acetylation of a hydroxyl group by an alkyl halide; esterification, amidation (e.g., carbodiimides or other catalyst mediated amidation), and/or reduction to an alcohol of a carboxyl moiety; acidic or basic condition deamidation of an asparagine and/or a glutamine; an acylation, an alkylation, an arylation, and/or an amidation reaction of an amino group such as the primary amino group of a proteinaceous molecule (e.g., a peptide) and/or the amino group of a lysine residue; halogenation and/or nitration of the phenolic moiety of a tyrosine; or a combination thereof. Examples where solubility of a proteinaceous molecule (e.g., a peptide) may be decreased include acylating a charged lysine residue and/or acetylating a carboxyl moiety of an aspartic acid and/or a glutamic acid.

In some embodiments, a cysteine may be eliminated from a proteinaceous molecule's (e.g., a peptide, an antibiological peptide) sequence, which may reduce cross linking via the cysteine's amino acid's free sulfhydryl moiety. A proteinaceous molecule (e.g., an antifungal peptide, an antibiological peptide) may possess an activity (e.g., an antibiological activity) in the form of one type of stereoisomer and/or as a mixed stereoisomeric composition. In some embodiments, a proteinaceous composition (e.g., a peptide composition, an antibiotic peptide composition) comprises proteinaceous molecule (e.g., a peptide, a peptide library) has not been purified (e.g., impure by comprising one or more peptides of unknown exact sequence), comprises a side chain that has not been de-blocked (i.e., comprises a blocked side chain), comprises a covalent attachment to the synthetic resin (e.g., has not been cleared from a synthetic resin) used to anchor the growing amino acid chain of a peptide, or a combination thereof (e.g., both blocked at a side chain and attached to a resin).

In an additional example, the secondary, tertiary and/or quaternary structure of a proteinaceous molecule may be modeled using techniques known in the art, including X-ray crystallography, nuclear magnetic resonance, computer based modeling, or a combination thereof to aid in the identification of active-site, binding site, and other residues for the design and production of a mutant form of a proteinaceous molecule (e.g., an enzyme) (Bugg, C. E. et al., 1993; Cohen, A. A. and Shatzmiller, S. E., 1993; Hruby, V. J., 1993; Moore, G. J., 1994; Dean, P. M., 1994; Wiley, R. A. and Rich, D. H., 1993). The secondary, tertiary and/or quaternary structures of a proteinaceous molecule may be directly determined by techniques such as X-ray crystallography and/or nuclear magnetic resonance to identify amino acids likely to effect one or more properties. Additionally, many primary, secondary, tertiary, and/or quaternary structures of proteinaceous molecules may be obtained using a public computerized database. An example of such a databank that may be used for this purpose comprises the Protein Data Bank (PDB), an international repository of the 3-dimensional structures of many biological macromolecules.

Computer modeling may be used to identify amino acids likely to affect one or more properties. Often, a structurally related proteinaceous molecule comprises primary, secondary, tertiary and/or quaternary structures that are evolutionarily conserved in the wild-type protein sequences of various organisms. The secondary, tertiary and/or quaternary structure of a proteinaceous molecule may be modeled using a computer to overlay the proteinaceous molecule's amino acid sequence, which may be also known as the “primary structure,” onto the computer model of a described primary, secondary, tertiary, and/or quaternary structure of another, structurally related proteinaceous molecule. Often the amino acids that may participate in an active site, a binding site, a transmembrane domain, the general hydrophobicity and/or hydrophilicity of a proteinaceous molecule, the general positive and/or negative charge of a proteinaceous molecule, etc, may be identified by such comparative computer modeling.

A selected proteinaceous molecule (e.g., an active peptide), may be modified to comprise functionally equivalent amino acid substitutions and yet retain the same or similar characteristics (e.g, an antibiological property). In embodiments wherein an amino acid of particular interest has been identified using such techniques, functional equivalents may be created using mutations that substitute a different amino acid for the identified amino acid of interest. Examples of substitutions of an amino acid side chain to produce a “functional equivalent” proteinaceous molecule are also known in the art, and may involve a conservative side chain substitution a non-conservative side chain substitution, or a combination thereof, to rationally alter a property of a proteinaceous molecule. Examples of conservative side chain substitutions include, when applicable, replacing an amino acid side chain with one similar in charge (e.g., an arginine, a histidine, a lysine); similar in hydropathic index; similar in hydrophilicity; similar in hydrophobicity; similar in shape (e.g., a phenylalanine, a tryptophan, a tyrosine); similar in size (e.g., an alanine, a glycine, a serine); similar in chemical type (e.g., acidic side chains, aromatic side chains, basic side chains); or a combination thereof. Conversely, when a change to produce a non-conservative substitution to alter a property of proteinaceous molecule, and still produce a “functional equivalent” proteinaceous molecule, these guidelines may be used to select an amino acid whose side-chains relatively non-similar in charge, hydropathic index, hydrophilicity, hydrophobicity, shape, size, chemical type, or a combination thereof.

Various amino acids have been given a numeric quantity based on the characteristics of charge and hydrophobicity, called the hydropathic index (Kyte, J. and Doolittle, R. F. 1982), which may be used as a criterion for a substitution (e.g., a substitution related to conferring or retaining a biological function). For example, the relative hydropathic character of the amino acid may determine the secondary structure of the resultant protein, which in turn defines the interaction of the protein with a ligand (e.g., a substrate) molecule. Similarly, in a proteinaceous molecule (e.g., a peptide, a polypeptide) whose secondary structure may not be a principal aspect of the interaction of the proteinaceous molecule (e.g., a peptide), position within the proteinaceous molecule (e.g., a peptide), and a characteristic of the amino acid residue may determine the interaction the proteinaceous molecule (e.g., a peptide) has in a biological system. An amino acid sequence may be varied in some embodiments. For example, certain amino acids may be substituted for other amino acids having a similar hydropathic index or score and still retain similar if not identical biological activity. The hydropathic index of the common amino acids are: Arg (−4.5); Lys (−3.9); Asn (−3.5); Asp (−3.5); Gln (−3.5); Glu (−3.5); His (−3.2); Pro (−1.6); Tyr (−1.3); Trp (−0.9); Ser (−0.8); Thr (−0.7); Gly (−0.4); Ala (+1.8); Met (+1.9); Cys (+2.5); Phe (+2.8); Leu (+3.8); Val (+4.2); and Ile (+4.5). Additionally, a value has also been given to various amino acids based on hydrophilicity, which may also be used as a criterion for substitution (U.S. Pat. No. 4,554,101). The hydrophilicity values for the common amino acids are: Trp (−3.4); Phe (−2.5); Tyr (−2.3); Ile (−1.8); Leu (−1.8); Val (−1.5); Met (−1.3); Cys (−1.0); Ala (−0.5); His (−0.5); Pro (−0.5+/−0.1); Thr (−0.4); Gly (O); Asn (+0.2); Gln (+0.2); Ser (+0.3); Asp (+3.0+/−0.1); Glu (+3.0+/−0.1); Arg (+3.0); and/or Lys (+3.0). In aspects wherein an amino acid may be conservatively substituted (i.e., exchanged) for an amino acid comprising a similar or same hydropathic index and/or hydrophilic value, the difference between the respective index and/or value may be generally within +/−2, within +/−1, and/or within +/−0.5. A biological functional equivalence may typically be maintained wherein an amino acid substituted (e.g., conservatively substituted). Thus, it is expected that isoleucine, for example, which has a hydropathic index of +4.5, can be substituted for valine (+4.2) or leucine (+3.8), and still obtain a proteinaceous molecule (e.g., a protein) having similar activity (e.g., a biologic activity). A lysine (−3.9) can be substituted for arginine (−4.5), and so on. These amino acid substitutions are generally based on the relative similarity of R-group substituents, for example, in terms of size, electrophilic character, charge, and the like. Although these are not the only such substitutions, the substitutions which take the foregoing characteristics into consideration, for example for a hydropathic index, include An alanine substituted with a Gly and/or a Ser; an arginine substituted with a Lys; an asparagine substituted with a Gln and/or a His; an aspartate substituted with a Glu; a cysteine substituted with a Ser; a glutamate substituted with an Asp; a glutamine substituted with an Asn; a glycine substituted with an Ala; a histidine substituted with an Asn and/or a Gln; an isoleucine substituted with a Leu and/or Val; a leucine substituted with an Ile and/or a Val; a lysine substituted with an Arg, a Gln, and/or a Glu; a methionine substituted with a Met, a Leu, a Tyr; a serine substituted with a Thr; a threonine substituted with a Ser; a tryptophan substituted with a Tyr; a tyrosine substituted with a Trp and/or a Phe; a valine substituted with a Ile and/or a Leu; or a combination thereof. In aspects wherein an amino acid may be non-conservatively substituted, the difference between the respective hydropathic index and/or hydrophilic value may be greater than +/−0.5, greater than +/−1, and/or greater than +/−2.

In certain embodiments, a functional equivalent may be produced by a non-mutation based chemical modification to an amino acid, a peptide, and/or a polypeptide. Examples of chemical modifications include, when applicable, a hydroxylation of a proline and/or a lysine; a phosphorylation of a hydroxyl group of a serine and/or a threonine; a methylation of an alpha-amino group of a lysine, an arginine and/or a histidine (Creighton, T. E., 1983); adding a detectable label such as a fluorescein isothiocyanate compound (“FITC”) to a lysine side chain and/or a terminal amine (Rogers, K. R. et al., 1999); covalent attachment of a poly ethylene glycol (Yang, Z. et al., 1995; Kim, C. et al., 1999; Yang, Z. et al., 1996; Mijs, M. et al., 1994); an acylatylation of an amino acid, particularly at the N-terminus; an amination of an amino acid, particularly at the C-terminus (Greene, T. W. and Wuts, P. G. M. “Productive Groups in Organic Synthesis,” Second Edition, pp. 309-315, John Wiley & Sons, Inc., USA, 1991); a deamidation of an asparagine or a glutamine to an aspartic acid or glutamic acid, respectively; a derivation of an amino acid by a sugar moiety, a lipid, a phosphate, and/or a farnysyl group; an aggregation (e.g., a dimerization) of a plurality of proteinaceous molecules, whether of identical sequence or varying sequences; a cross-linking of a plurality of proteinaceous molecules using a cross-linking agent [e.g., a 1,1-bis(diazoacetyl)-2-phenylethane; a glutaraldehyde; a N-hydroxysuccinimide ester; a 3,3′-dithiobis(succinimidyl-propionate); a bis-N-maleimido-1,8-octane]; an ionization of an amino acid into an acidic, basic or neutral salt form; an oxidation of an amino acid; or a combination thereof of any of the forgoing. Such modifications may produce an alteration in a property of a proteinaceous molecule. For example, a N-terminal glycosylation may enhance a proteinaceous molecule's stability (Powell, M. F. et al., 1993). In an additional example, substitution of a beta-amino acid isoserine for a serine may enhance the aminopeptidase resistance a proteinaceous molecule (Coller, B. S. et al., 1993).

A proteinaceous molecule may comprise a proteinaceous molecule longer or shorter than the wild-type amino acid sequence(s). For example, an enzyme comprising longer or shorter sequence(s) may be encompassed, insofar as it retains enzymatic activity. In some embodiments, a proteinaceous molecule may comprise one or more peptide and/or polypeptide sequence(s). In certain embodiments, a modification to a proteinaceous molecule may add and/or subtract one or two amino acids from a peptide and/or polypeptide sequence. In other embodiments, a change to a proteinaceous molecule may add and/or remove one or more peptide and/or polypeptide sequence(s). Often a peptide or a polypeptide sequence may be added or removed to confer or remove a specific property from the proteinaceous molecule, and numerous examples of such modifications to a proteinaceous molecule are described herein, particularly in reference to fusion proteins. In a particular example, the native OPH of Pseudomonas diminuta may be produced with a short amino acide sequence at its N-terminas that promotes the exportation of the protein through the cell membrane and later cleaved. Thus, in certain embodiment, this signal sequence's amino acid sequence may be deleted by genetic modification in the DNA construction placed into Escherichia coli host cells to enhance its production.

As used herein, a “peptide” comprises a contiguous molecular sequence from about 3 to about 100 amino acids in length. A sequence of a peptide may comprise about 3 to about 100 amino acids in length. As used herein a “polypeptide” comprises a contiguous molecular sequence about 101 amino acids or greater. Examples of a sequence length of a polypeptide include about 101 to about 10,000 amino acids.

As used herein a “protein” may comprise a proteinaceous molecule comprising a contiguous molecular sequence three amino acids or greater in length, matching the length of a biologically produced proteinaceous molecule encoded by the genome of an organism.

Removal of one or more amino acids from a proteinaceous moleculee's sequence may reduce or eliminate a detectable property such as enzymatic activity, binding activity, etc. However, a longer sequence, particularly a proteinaceous molecule, may consecutively and/or non-consecutively comprises and/or even repeats one or more sequences of a proteinaceous molecule (e.g., a repeated enzymatic sequence, a repeated antimicrobial peptide sequence), including but not limited to those disclosed herein. Additionally, fusion proteins may be bioengineered to comprise a wild-type sequence and/or a functional equivalent of a proteinaceous molecule's sequence and an additional peptide and/or polypeptide sequence that confers a property and/or function.

1. Lipolytic Enzymes Functional Equivalents

An example of a functional equivalent includes a lipolytic enzyme functional equivalent. Using recombinant DNA technology, wild-type and mutant forms of numerous lipolytic genes have been expressed in various cell types and expression systems, for further characterization and analysis, as well as large scale production of lipolytic enzymes for industrial and/or commercial use. Often signaling sequences are added, deleted and/or modified to redirect an expressed enzyme's targeting to extracellular secretion to allow rapid purification from cellular material, and additional sequences, particularly tags (e.g., a poly His tag) are added to aid in purification. In other cases, an enzyme may be targeted to the cell surface and/or to intercellular expression. Codon optimization may be used to enhance yield of enzyme produced in a host cell. For example, mutations converting one or more residues of a protease cleavage site may enhance resistance to protease digestion. In one example, chymotrypsin cleavage site residues 149-156 identified in Pseudomonas glumae lipase may be converted into a proline, an arginine, and/or other residue(s) for enhance enzyme stability against protease inactivation.

To improve stability, particularly thermostability, a mutation may be made that mimic the differences between a thermophilic lipolytic enzyme and a psychrophilic and/or a mesophilic lipolytic enzyme. Examples of such a mutation to improve stability, such as thermostability, comprises ones that improve the hydrophobic core packaging (i.e., enhance the ratio of the residues' volume within the van der Waals distances to total residues' volume; reduce the total enzyme surface-to-volume ratio); increases the percentage of arginine as charged residues, as arginine forms stabilizing ion-pairs; mutating a peptide bond that are liable to spontaneous and/or chemical (i.e., asn-gln, asp-pro) breakage; replaces a residue susceptible to oxidation, such as a methionine (e.g., a met with a leu) and aromatic residues, particularly those on the surface; and make such changes isomorphic (e.g., by use of a residue of similar size during substitution mutation) to prevent voids from being created [In “Engineering of/with Lipases” (F. Xavier Malcata., Ed.) pp. 193-197, 1996].

The X-ray crystal structures for various lipolytic enzymes (e.g., a Rhizomucor miehei lipase, a Humicola lanugnosa lipase, a Penicillium camemberti lipase, a Geotrichum candidum lipase, a human pancreatic lipase, a Fusarium solani cutinase, a Psuedomonas glumae lipase, a human nonpancreatic phospholipase A₂, a Naja Naja atra phospholipase A₂) have been solved, allowing comparison of lipolytic enzymes' structures and identification residues involved in function [In “Advances in Protein Chemistry, Volume 45 Lipoproteins, Apolipoproteins, and Lipases.” (Anfinsen, C. B., Edsall, J. T., Richards, Frederic, R. M., Eisenberg, D. S., and Schumaker, V. N. Eds.) Academic Press, Inc., San Diego, Calif., pp. 1-152, 1994; “Lipases their Structure, Biochemistry and Application” (Paul Woolley and Steffen B. Peterson, Eds.), pp. 1-243-270, 337-354, 1994.]. For example, comparison of lipolytic enzymes has identified interfacial activation induced conformational changes in the lid structure of many enzymes producing increases in hydrophobic surface area of the enzyme and formation of an oxyanion transition state binding site (“oxyanion hole”) that promotes catalysis. In contrast, a cutinase lacks a lid structure and has a preformed oxyanion hole, so it typically does not use interfacial activation for lipolytic activity (Martinez, C. et al., 1994; Nicolas, A. et al., 1996).

The availability of these crystal structures and computer modeling of sequences onto existing crystal structures allows targeted mutations and alterations to be made to residues identified as belonging to regions of the proteinaceous molecule (e.g., an enzyme) with specific functions (e.g., surface residues for solubility and/or ligand interactions, binding site residues, lid domain residues, etc.) For example, a cutinase Arg196Glu and Arg17Glu surface residues mutations improved stability in lithium dodecylsulphate, by mutating the charged surface residues to ones that are similarly charged as the detergent's hydrophilic head group, reducing detergent binding that destabilizes the enzyme. Ligand (e.g., substrate) preference may be changed by alterations to binding site residue(s) and/or residue(s) of domains near the binding site. For example, the preference for a cutinase for esters of about 4 to about 5 carbon fatty acids was shifted to esters of about 7 to about 8 carbon fatty acids by a binding site A85F mutation. In another example, a Phe139Trp mutation of the lid domain of a Candida antartica lipase improved activity against tributyrine substrate about 4-fold after comparison to the crystal structures of the more active lipases from a Rhizomucor miehei and a Humicola lanuginosa. In an additional example, enantioselectivity for a Humicola lanuginosa lipase was increased for 1-heptyl 2-methyldcanoate and decreased for phenyl 2-methyldecanoate by mutation to alter the open-lid conformation's electrostatic stability (In “Engineering of/with Lipases” (F. Xavier Malcata., Ed.) pp. 197-202, 1996).

In a further example, a Lipolase™ and a Lipolase Ultra™ are industrial lipases produced by multiple mutations to improve enzyme properties of temperature stability, proteolytic cleavage resistance, oxidation resistance, detergent resistance, and pH optimization. These lipases are mutated forms of the lipase isolated from a Humicola lanuginsa, where negatively charged residue(s) on the lid domain were replaced with positive and/or hydrophobic residue(s) (e.g., D96L) to reduce repulsion of negatively charged FAs and/or surfactant(s) associated with lipid(s), resulting in about 4 to about 5 fold or greater improvement in multicycle activity tests. Mutations at a Savinase™ cleavage sites (e.g., residues 160-169 and 206-215) also improved resistance to a proteolytic digestion. As an alternative to such rational design of mutations based on comparison of similar enzymes sequences, crystal structures, etc., bulk mutations via random mutation libraries may be used directed domain sequences implicated with stability and/or activity (e.g., lid domain in a lipolytic enzyme, an active site region) to generate large numbers of mutants under selective screening protocols to mimic evolution and identify a modified enzyme (In “Engineering of/with Lipases” (F. Xavier Malcata., Ed.) pp. 203-217, 1996).

Additional non-limiting examples of such recombinant expression of lipolytic enzymes, particularly enzymes having one or more mutations from the wild-type sequence (e.g., tags, signal sequences, mutations altering activity, etc.), are shown on the Table below.

TABLE 5
Examples of Recombinantly Expressed Lipolytic Enzymes

| Lipolytic Enzyme | Characteristics | Source/Host Cell | References |
|---|---|---|---|
| Carboxylesterase | lipA gene; preference for a short | Archaeoglobus fulgidus | Rusnak, M. et |
| | chain FA ester; optimum activity | DSM 4304/Escherichia | al., 2005. |
| | 70° C., pH 10-11 | coli | |
| Carboxylesterase | broad specificity, preference for | Sulfolobus solfataricus P1/ | Park, Y. J. et |
| | a C8 FA ester; optimums 85° C., | Escherichia coli | al., 2006. |
| | pH 8.0; detergent, urea and | | |
| | organic solvent resistant | | |
| Carboxylesterase | optimums 60° C., pH 7.5; Ca²⁺ | Thermotoga maritima | Kakugawa, S. |
| | dependent | (tm0053)/Escherichia coli | et al., 2007. |
| | | expressed as N-terminal | |
| | | hydrophobic region | |
| | | truncation | |
| Carboxylesterase | preference for a C6 or less FA | Pseudomonas fluorescens/ | Choi, G. S. et |
| | ester | Escherichia coli | al., 2003. |
| | | expression as a fusion | |
| | | protein with a N-terminal | |
| | | hexahistidine tag | |
| Carboxylesterase | active at 70° C., pH 7.1; some | Bacillus acidocaldarius/ | Manco, G. et |
| | enantioselectivity; strong | Escherichia coli | al., 1998. |
| | preference for a short chain FA | | |
| | ester | | |
| Carboxylesterase | EstA gene | Burkholderia gladioli/ | Breinig, F. et |
| | | Saccharomyces | al., 2006. |
| | | cerevisiae, expressed as | |
| | | fusion protein on cell wall | |
| Carboxylesterase | preference for a short chain FA | Pseudomonas aeruginosa | Pesaresi, A. et |
| | ester optimum activity 55° C., pH | PAO1/Escherichia coli | al., 2005. |
| | 9.0 | | |
| Carboxylesterase | optimum activity pH 6.5-7.0; | Sulfolobus solfataricus | Morana, A. et |
| | preference for a C2 to C8 short | strain MT4/Escherichia | al., 2002. |
| | chain FA ester | coli | |
| Carboxylesterase | estB gene; preference for a C2 | Burkholderia gladioli/ | Petersen, E. I. |
| | to C6 short chain FA ester | Escherichia coli | et al., 2001. |
| Carboxylesterase | EST2 gene; active at 70° C., pH | Archaeoglobus fulgidus/ | Manco, G. et |
| | 7.1 | Escherichia coli | al., 2000. |
| Carboxylesterase | lip8 gene; selective against a | Pseudomonas aeruginosa | Ogino, H. et |
| | short chain FAs ester (e.g., a | LST-03/Pseudomonas | al., 2004. |
| | methyl ester) | aeruginosa LST-03 | |
| Carboxylesterase | Thermoacidophilic | Sulfolobus shibatae/ | Huddleston, S. |
| | | | et al., 1995. |
| Carboxylesterase | stable at 90° C.; activity against a | Sulfolobus shibatae | Ejima, K. et al., |
| | C2 to C16 FA ester, though not | DSM5389/Escherichia | 2004. |
| | discernibly active against | coli JM109 | |
| | triacylglycerol | | |
| Carboxylesterase | Optimum activity 70° C.; | Alicyclobacillus (formerly | De Simone, G. |
| | preference for an about 6 C to | Bacillus) acidocaldarius/ | et al., 2000. |
| | about 8 C FA ester | Escherichia coli strain 834 | |
| | | (DE3) | |
| Carboxylesterase | active between 30° C. to −90° C.; | Environment source | Rhee, J. K. et |
| | optimum activity pH 6.0, good | library/Escherichia coli | al., 2005. |
| | activity pH 5.5-7.5; preference | | |
| | for a 10 C or shorter FA ester | | |
| Carboxylesterase | estD gene; optimum activity | Thermotoga maritima/ | Levisson, M. et |
| | 95° C., pH 7; preference for a C4 | Escherichia coli | al., 2007. |
| | to a C8 short chain FA ester | | |
| Carboxylesterase/ | Est3 gene; broad substrate | Sulfolobus solfataricus P2/ | Kim, S. and |
| Lipase | range - a C2 to C16 FA; | Escherichia coli | Lee, S. B., |
| | optimum about 80° C., about pH | | 2004. |
| | 7.4; some enantioselectivity | | |
| Carboxylesterase/ | p65 enzyme; preference for a | Mycoplasma | Schmidt, J. A. |
| Lipase | short chain fatty acid; optimums | hyopneumoniae/ | et al., 2004. |
| | greater than 39° C., pH 9.2-10.2 | Escherichia coli expressed | |
| | | as glutathione S- | |
| | | transferase (GST)-p65 | |
| | | fusion protein after | |
| | | truncation of signal | |
| | | sequence | |
| Carboxylesterases/ | many isolates selective for a | Fosmid and microbial | Lee, S. W. et |
| Lipases | short over a long chain FA ester | DNA from forest | al., 2004. |
| | | topsoil/Escherichia coli | |
| | | secretion expression of 6 | |
| | | lipolytic enzymes with | |
| | | homology to hormone | |
| | | sensitive lipase and | |
| | | identified by library | |
| | | screening of tributyrin | |
| | | hydrolyzing isolates. | |
| Carboxylesterase/ | SSoNDelta and SSoNDeltalong | Sulfolobus solfataricus/ | Mandrich, L. et |
| Lipases | genes; optimums pH 7.2, 70° C. | Escherichia coli strains | al., 2007. |
| | and pH 6.5, 85° C., respectively; | Top10 and BL21(DE3) | |
| | both active against a C4 to C18 | strains | |
| | FA ester | | |
| Carboxylesterases/ | 3 enzymes expressed, | Myxococcus xanthus/ | Moraleda- |
| Lipases | preference for a short chain FA | Escherichia coli BL21 Star | Muñoz, A. and |
| | ester | (DE3) expressed as lacZ | Shimkets, L. J., |
| | | fusion protein in | 2007. |
| | | (pET102/D-TOPO) vector | |
| | | system | |
| Carboxylesterase/ | Met(423)Ile, Met(423) Ile, | Rattus norvegicus/COS- | Wallace, T. J. et |
| Sterol esterase | Thr(444) Met mutations to mimic | 7 expression of mutant | al., 2001. |
| | sequence of cholesterol | enzyme | |
| | esterase in carboxylesterase | | |
| | conferred cholesterol esterase | | |
| | activity | | |
| Lipase | | Candida antarctica, A. oryzae | Tamalampudi, S. |
| | | niaD300/ | et al., 2007. |
| | | Aspergillus oryzae | |
| | | expressed in whole cells | |
| | | under improved glaA and | |
| | | pNo-8142 promoters and | |
| | | plasmids pNGA142 and | |
| | | pNAN8142, respectively, | |
| | | as fusion proteins with | |
| | | secretion signals and | |
| | | FLAG tags | |
| Lipase | Hepatic | Homo sapiens/rabbits | Rizzo, M. et al., |
| | | (transgenic) | 2004. |
| Lipase | | Geobacillus sp. strain T1/ | Rahman, R. N. |
| | | Escherichia coli Top10, | et al., 2005. |
| | | TG1, XL1-Blue, | |
| | | BL21(De3)plysS, and | |
| | | Origami B, secretion | |
| | | expression via plasmid | |
| | | pGEX/T1S and pJL3 | |
| | | vectors | |
| Lipase | optimums 60 to 65° C., pH 9.0 to | Bacillus | Kim, H. K. et |
| | 10.0 | stearothermophilus L1/ | al., 1998. |
| | | Escherichia coli, Ala | |
| | | replaces the 1st Gly in the | |
| | | GlyXaaSerXaaGly | |
| | | sequence | |
| Lipase | bile salt stimulated | Homo sapiens/Pichia | Sahasrabudhe, A. V. |
| | | pastoris secretion | et al., |
| | | expression | 1998. |
| Lipase | optimum 68° C.; stability noted at | Bacillus | Kim, M. H. et |
| | 55° C.; stability increased 8° C.⁺ by | stearothermophilus L1/ | al., 2000. |
| | Ca²⁺. | Escherichia coli secretion | |
| | | expression via pET-22b(+) | |
| | | vector | |
| Lipase | stable at 60° C., pH 8.0; active at | GeoBacillus | Abdel-Fattah, Y, R., |
| | 100° C. | thermoleovorans Toshki/ | and |
| | | Escherichia coli via T7 | Gaballa AA., |
| | | promoter and pET 15b | 2008. |
| | | vector | |
| Lipase | bile salt stimulated | Homo sapiens/ | Downs, D. et |
| | | Escherichia coli via T7 | al., 1994. |
| | | expression system, N- | |
| | | terminus truncated. | |
| Lipase | | Homo sapiens (hepatic | Rashid, S. et |
| | | lipase)/rabbit transfected | al., 2003. |
| | | with adenovirus | |
| | | expressing lipase gene | |
| Lipase | alkaline lipase | Penicillium cyclopium | Wu, M. et al., |
| | | PG37/Escherichia coli | 2003. |
| | | expression in pET-30a | |
| Lipase | microsomal; S221A, E354A, and | Homo sapiens/SF-9 cells | Alam, M. et al., |
| | H468A mutants inactive; N- | secretion expression | 2002. |
| | glycosylation site N79A mutant | | |
| | not glycosylated; C-terminal | | |
| | endoplasmic reticulum retrieval | | |
| | signal deletion prevented | | |
| | secretion | | |
| Lipase | | Rhizopus oryzae/ | Washida, M. et |
| | | Saccharomyces | al., 2001. |
| | | cerevisiae expressed as a | |
| | | cell surface fusion protein | |
| | | of the pre-alpha-factor | |
| | | leader sequence and a C- | |
| | | terminal alpha-agglutinin | |
| | | segment including a | |
| | | glycosylphosphatidylinositol- | |
| | | anchor | |
| Lipase | bile salt-stimulated | Homo sapiens/Pichia | Murasugi, A. et |
| | | pastoris, expressed | al., 2001. |
| | | underAOX1 gene | |
| | | promoter, C-terminus | |
| | | truncated to enhance | |
| | | secretion | |
| Lipase | | Candida antarctica/ | Gustavsson, M. |
| | | Pichia pastoris, expressed | et al., 2001. |
| | | as a cellulose-binding | |
| | | domain fusion protein for | |
| | | immobilization onto | |
| | | cellulose | |
| Lipase | Thermostable | Bacillus | Sinchaikul, S. |
| | | stearothermophilus P1/ | et al., 2002. |
| | | Escherichia coli | |
| Lipase | CpLIP2 | Candida parapsilosis/ | Neugnot, V. et |
| | | Saccharomyces | al., 2002. |
| | | cerevisiae, including C- | |
| | | terminal histidine tag | |
| Lipase | L167V mutation increased | Burkholderia cepacia KWI- | Yang, J. et al., |
| | preference for a short chain | 56/in vitro expression | 2002. |
| | ester; F119A/L167M mutation | with Escherichia coli S30 | |
| | increased preference for long- | transcription/translation | |
| | chain ester | system | |
| Lipase | preference for C2-C4 short | Acinetobacter species SY- | Han, S. J. et al., |
| | chain esters; able to hydrolyze a | 01/Bacillus subtilis 168 | 2003. |
| | wide range of esters and | | |
| | monoesters; optimum 50° C., pH | | |
| | 10; stable pH 9-11, optimum | | |
| Lipase | | Serratia marcescens/S. | Idei, A. et al., |
| | | marcescens via lipA gene | 2002. |
| | | in pUC19 coexpressed | |
| | | with an ATP-binding | |
| | | cassette (ABC) exporter to | |
| | | enhance secretion in a | |
| | | feed batch system | |
| Lipases | endothelial cell-derived, several | Homo sapiens/Homo | Ishida, T. et al., |
| | isoforms | sapiens tissue cells, | 2004. |
| | | including endothelial cells, | |
| | | secreted isoform active. | |
| Lipase | lip1 | Kurtzmanomyces sp. I-11/ | Kakugawa, K. |
| | | Pichia pastoris | et al., 2002. |
| Lipase | optimums 50° C., pH 7.0; stable | Acinetobacter | Dharmsthiti, S. |
| | at 37° C.; stable in the presence | calcoaceticus LP009/ | et al., 1998. |
| | of 0.1% Triton X-100, Tween-80 | Aeromonas sobria | |
| | and/or Tween-20, enhanced by | | |
| | Fe³⁺ | | |
| Lipases | CdLIP1, CdLIP2 and CdLIP3, | Candida deformans CBS | Bigey, F. et al., |
| | EMBL Accession Nos | 2071/Saccharomyces | 2003. |
| | AJ428393, AJ428394 and | cerevisiae | |
| | AJ428395 | | |
| Lipase | BTL2 gene; stable in the | Bacillus | Quyen, D. T. et |
| | presence of detergents and | thermocatenulatus/Pichia | al., 2003. |
| | organic solvents | pastoris GS115 secreted | |
| | | enzyme | |
| Lipase | Thermoalkaophilic | Bacillus | Schlieben, |
| | | thermocatenulatus/ | N. H. et al., |
| | | Escherichia coli secretion | 2004. |
| | | expression of His-tagged | |
| | | enzyme for metal affinity | |
| | | chromatography | |
| | | purification | |
| Lipase | | Y. lipolytica/Yarrowia | Nicaud, J. M. et |
| | | lipolytica expression by | al., 2002. |
| | | the hp4d promoter in fed | |
| | | batch culture | |
| Lipase | | Bacillus subtilis/ | Sánchez, M. et |
| | | Escherichia coli, | al., 2002. |
| | | Saccharomyces | |
| | | cerevisiae and Bacillus | |
| | | subtilis via pBR322, | |
| | | YEplac112 and pUB110- | |
| | | derived vectors. | |
| Lipase | lipF gene, effective on a short | Mycobacterium | Zhang, M. et |
| | chain FAs ester | tuberculosis/Escherichia | al., 2005. |
| | | coli, expressed as fusion | |
| | | protein, site directed | |
| | | mutation of Ser90, | |
| | | Glu189, His219 active site | |
| | | residues. | |
| Lipase | | Oryza sativa/Escherichia | Kim, Y., 2004. |
| | | coli expression by a pET | |
| | | expression system, | |
| | | enzyme associated with | |
| | | cell rather than secreted | |
| Lipases | ipla2epsilon, ipla2zeta, and | Homo sapiens/ | Jenkins, C. M. |
| | ipla2eta | Spodoptera frugiperda | et al., 2005. |
| | | SF9 cell | |
| Lipase | lipB52 gene; optimums: 40° C., | Pseudomonas fluorescens/ | Jiang, Z. et al., |
| | pH 8.0 | Pichia pastoris KM71, | 2005. |
| | | secreted via pPIC9K | |
| | | vector expression | |
| Lipase | lip1 gene; thermostable | Candida rugosa/Pichia | Chang, S. W. et |
| | | after conversion of 19 | al., 2005. |
| | | CTG non-universal | |
| | | codons into universal | |
| | | codons to enhance | |
| | | enzyme production. | |
| Lipase | lip2 gene | Yarrowia lipolytica/ | Fickers, P. et |
| | | Yarrowia lipolytica strain | al., 2005. |
| | | LgX64.81 batch of fed | |
| | | batch extracellular | |
| | | expression | |
| Lipase | | Bacillus | Ahn, J. O. et al., |
| | | stearothermophilus L1/ | 2004. |
| | | Saccharomyces | |
| | | cerevisiae secreted under | |
| | | the galactose-inducible | |
| | | GAL10 promoter as a | |
| | | cellulose-binding domain | |
| | | fusion protein, the alpha- | |
| | | amylase signal peptide | |
| | | after fed batch production | |
| Lipase | | Rhizopus oryzae/Pichia | Resina, D. et |
| | | pastoris expressed by | al., 2005. |
| | | FLD1 promoter in fed | |
| | | batch culture. | |
| Lipase | specificity for a long chain FA; | Lycopersicon esculentum | Matsui, K. et |
| | optimum pH 8.0 | L/Escherichia coli | al., 2004. |
| | | SG13009 [pREP4], M15 | |
| | | [pREP4], Y1090, or | |
| | | Origami (DE3) strains | |
| | | used for intercellular | |
| | | expression | |
| Lipase | optimum 40° C., active up to | Geobacillus sp. | Li, H., Zhang |
| | 90° C.; optimum pH 7.0-8.0, pH | TW1/Escherichia coli as | X. et al., 2005. |
| | range 6.0-9.0; stable in 0.1% | glutathione S-transferase | |
| | detergents such as Tween 20, | fusion protein. | |
| | Chaps, Triton X-100; enhanced | | |
| | by Ca²⁺, Mg²⁺, Zn²⁺, Fe²⁺ and/or | | |
| | Fe³⁺; inhibited by Cu²⁺, Mn²⁺, | | |
| | and Li⁺ | | |
| Lipase | alip1 gene; optimums 30° C., pH | Arxula adeninivorans/ | Böer, E. et al., |
| | 7.5; selective toward a medium | Arxula adeninivorans | 2005. |
| | chain FAs ester of 8 to 10 | using strong TEF1 | |
| | carbons over a short or a long | promoter | |
| | chain FA ester | | |
| Lipase | lipJ02 gene and lipJ03 gene; | Environmental DNA/ | Jiang, Z. et al., |
| | optimums 30° C. and 35° C., | Pichia pastoris KM71 via | 2006. |
| | respectively; function at pH 8.0 | pPIC9K vector secretion | |
| | | expression. | |
| Lipase | activators, Ca²⁺, K⁺, and Mg²⁺, 7 mM | Bacillus subtilis strain | Ma, J. et al., |
| | sodium taurocholate; | IFFI10210/B. subtilis | 2006. |
| | inhibitors, Fe²⁺, Cu²⁺, and Co²⁺, | strain IFFI10210 via | |
| | 10 mM sodium taurocholate | pBSR2 plasmid | |
| | | expression | |
| Lipase | Calip4 gene, selective for an | Candida albicans/ | Roustan, J. L. |
| | unsaturated over a saturated FA | Saccharomyces | et al., 2005. |
| | | cerevisiae secretion via | |
| | | codon change from CUG | |
| | | serine codon into a | |
| | | universal codon. | |
| Lipase | glip1 gene | Arabidopsis thaliana/ | Oh, I. S. et al., |
| | | Escherichia coli, secretion | 2005. |
| | | expression via a pGEX6P- | |
| | | 1 vector | |
| Lipase | | Geobacillus sp. strain T1/ | Rahman, R. N. |
| | | Escherichia coli Origami B | et al., 2005. |
| | | strain secretion after | |
| | | recombinant plasmid | |
| | | pGEX/T1S and pJL3 | |
| | | vector expression. | |
| Lipase | lipA gene | Serratia marcescens 8000 | Kawai, E. et |
| | | mutated by N-methyl-N′- | al., 2001. |
| | | nitro-N-nitrosoguanidine | |
| | | into a high expression | |
| | | strain GE14, extracellular | |
| | | enzyme | |
| Lipase | | Candida rugosa/Pichia | Passolunghi, |
| | | pastoris enzyme secretion | S. et al., 2003. |
| | | in batch culture, also | |
| | | expressed as a green | |
| | | fluorescent fusion protein | |
| | | to tract extracellular | |
| | | secretion pathway. | |
| Lipase | Ala substituted for the 1st Gly of | Geobacillus sp. strain T1/ | Leow, T. C. et |
| | the GlyXaaSerXaaGly substrate | E. coli intercellular | al., 2004. |
| | binding site; optimums 65° C., pH | expression under araBAD, | |
| | 9.0; active range pH 6 to 11 | T7, T7 lac, and tac | |
| | | promoters in pBAD, | |
| | | pRSET, pET, and pGEX | |
| | | expression vectors. | |
| Lipase | | Bacillus subtilis/ | Narita, J. et al., |
| | | Escherichia coli via cell | 2006. |
| | | surface expression as a | |
| | | FLAG peptide-fusion | |
| | | protein | |
| Lipase | chimeric enzyme of 3 lipases; | Candida antarctica ATCC | Suen, W. C. et |
| | active at 45° C., a higher | 32657 + Hyphozyma sp. | al., 2004. |
| | temperature than parent | CBS 648.91 + | |
| | enzymes | Crytococcus tsukubaensis | |
| | | ATCC 24555/ | |
| | | Saccharomyces | |
| | | cerevisiae | |
| Lipase | tglA gene | Aspergillus oryzae | Kaieda, M. et |
| | | niaD300/Aspergillus | al., 2004. |
| | | oryzae expression under a | |
| | | glaA promoter of plasmid | |
| | | pNGA142, whole-cells | |
| | | immobilized to biomass- | |
| | | support particles. | |
| Lipase | Ca²⁺-dependent, Mn²⁺ and Sr²⁺ | Pseudomonas sp./ | Rashid, N. et |
| | also enhances activity; | Escherichia coli | al., 2001. |
| | preference for a C10 FA and a 1 | | |
| | and/or 3 ester glycerol position | | |
| | ester; optimum 35° C. | | |
| Lipase | | Thermomyces | Prathumpai, W. |
| | | lanuginosus/Aspergillus | et al., 2004. |
| | | niger (strain NW 297-14 | |
| | | and NW297-24) | |
| | | expressed with Aspergillus | |
| | | oryzae TAKA amylase | |
| | | promoter, bound to cell | |
| | | wall after production | |
| Lipase | lipA gene | Pseudomonas fluorescens | Kojima, Y., et |
| | | HU380/Escherichia coli, | al., 2003. |
| | | refolded from inclusion | |
| | | bodies | |
| Lipase | Liver lysosomal acid lipase | Homo sapiens/ | Zschenker, O. |
| | | Spodoptera frugiperda | et al., 2004. |
| | | insect cells by expression | |
| | | without the signal peptide | |
| | | sequence; mutation G50A | |
| | | inhibit activity possibly by | |
| | | preventing cleavage of | |
| | | preprotein | |
| Lipase | | Phlebotomus papatasi/ | Belardinelli, M. |
| | | Escherichia coli via | et al., 2005. |
| | | pQE30 vector expression. | |
| Lipase | active at 65° C. when absorbed | Bacillus | Palomo, J. M. |
| | onto hydrophobic support | thermocatenulatus (BTL2)/ | et al., 2004. |
| | | Escherichia coli | |
| | | expressed, secreted | |
| | | enzyme absorbed onto | |
| | | hydrophobic support | |
| | | (octadecyl-Sepabeads) | |
| | | increased thermostability | |
| | | 10° C. | |
| Lipase | | Rhizopus oryzae/Pichia | Resina, D. et |
| | | pastoris secretion | al., 2004. |
| | | expression under the | |
| | | formaldehyde | |
| | | dehydrogenase 1 | |
| | | promoter | |
| Lipase | | Homo sapiens | Broedl, U. C. et |
| | | (endothelial)/transgenic | al., 2004. |
| | | mice | |
| Lipase | | Candida parapsilosis/ | Brunel, L. et |
| | | Pichia pastoris feed batch | al., 2004. |
| | | secretion expression by a | |
| | | methanol inducible alcohol | |
| | | oxidase 1 gene | |
| Lipase | | Homo sapiens (bile salt- | Trimble, R. B. |
| | | stimulated lipase)/Pichia | et al., 2004. |
| | | pastoris secreted as | |
| | | glycoprotein | |
| Lipase | optimums pH 8.0, 29° C.; active | Pseudomonas fragi strain | Alquati, C. et |
| | at 10° C. and 50° C.; 3D computer | IFO 3458/Escherichia | al., 2002. |
| | modeling against other lipases | coli SG13009 intercellular | |
| | verified catalytic triad: S83, | expression | |
| | D238 and H260, and oxyanion | | |
| | hole: L17, Q84 | | |
| Lipase | TliA gene | Pseudomonas fluorescens/ | Song, J. K. et |
| | | Serratia marcescen | al., 2007. |
| | | coexpression of cognate | |
| | | ABC transporter improved | |
| | | production/secretion using | |
| | | pTliDEFA-223 plasmid. | |
| Lipase | lipI gene | Galactomyces geotrichum | Fernández, L. |
| | | BT107/Pichia pastoris | et al., 2006. |
| | | secretion expression | |
| Lipase | optimums 40° C., pH 7.0 to 8.0; | Geobacillus sp. TW1/ | Li, H., and |
| | active up to 90° C. at pH 7.5; | Escherichia coli | Zhang X., |
| | stable at pH 6.0 to 9.0; stable in | expression as a | 2005. |
| | 0.1% detergents such as Tween | glutathione S-transferase | |
| | 20, Chaps and/or Triton X-100; | fusion protein | |
| | activity enhanced by Ca²⁺, Mg²⁺, | | |
| | Zn²⁺, Fe²⁺ and/or Fe³⁺, inhibited | | |
| | by Cu²⁺, Mn²⁺, and/or Li⁺ | | |
| Lipase | Gastric | Canis domesticus/corn | Zhong, Q. et |
| | | transgenic expression | al., 2006. |
| Lipase | BTL2 gene | Bacillus | Rúa, M. L. et |
| | | thermocatenulatus/ | al., 1998. |
| | | Escherichia coli cellular | |
| | | expression as fusion | |
| | | protein with OmpA | |
| | | outermembrane signal | |
| | | peptide in pCYT-EXP1 | |
| | | (pT1) expression vector | |
| Lipase | hybrid protein lost | Staphylococcus aureus | Nikoleit, K. et |
| | phospholipase activity but | NCTC8530 + | al., 1995. |
| | retained Ca²⁺ stimulation relative | Staphylococcus hyicus/ | |
| | to S. hyicus enzyme | Staphylococcus carnosus, | |
| | | secretion expression of a | |
| | | hybrid lipase having S. hyicus | |
| | | 146 residues) | |
| Lipase | lipCE gene; optimum 30° C. and | Environmental source | Elend, C. et al., |
| | pH 7.0; active at −5° C.; | isolation/Escherichia coli, | 2007. |
| | preference for a C10 FA ester, | refolded from inclusion | |
| | but large range of substrates; | bodies | |
| | steriospecific for (R)-ibuprofen | | |
| | esters | | |
| Lipase | optimum 75° C. | Bacillus thermoleovorans | Cho, A. R. et |
| | | ID-1/Escherichia coli | al., 2000. |
| | | expression via T7 | |
| | | promoter in pET-22b(+) | |
| | | vector | |
| Lipase | bile salt inhibited | Homo sapiens/Pichia | Sebban- |
| | | pastoris secretion | Kreuzer, C. et |
| | | expression via a pPIC9K | al., 2006. |
| | | vector | |
| Lipase | | Rhizopus oryzae/Pichia | Resina, D. et |
| | | pastoris expression under | al., 2007. |
| | | the formaldehyde | |
| | | dehydrogenase promoter | |
| | | in fed-batch cultivation | |
| Lipase | | Thermomyces | Haack, M. B. et |
| | | lanuginosus/Aspergillus | al., 2007. |
| | | oryzae expression in | |
| | | batch and fed-batch | |
| | | cultivation | |
| Lipase | | Aspergillus niger F044/ | Shu, Z. Y. et al., |
| | | Escherichia coli | 2007. |
| | | BL21(De3), refolded for | |
| | | activity after expression | |
| Lipase | Lysosomal acid | Homo sapiens/Homo | Pariyarath, R. |
| | | sapiens HeLa cells | et al., 1996. |
| | | expression via vaccinia T7 | |
| | | system | |
| Lipase | Hepatic | Homo sapiens/mice | Dugi, K. A. et |
| | | transgenic expression | al., 1997. |
| Lipase | | Candida rugosa/Pichia | Chang, S. W. et |
| | | pastoris, expression of a | al., 2006^{A}. |
| | | N-terminal peptide | |
| | | truncated with 18 non- | |
| | | universal CTG codons | |
| | | converted to TCT | |
| | | improved expression 52- | |
| | | fold | |
| Lipase | CtLIP gene; preference for 2- | Candida thermophila/ | Thongekkaew, J., |
| | position esters, optimum 55° C. | Saccharomyces | Boonchird C., |
| | | cerevisiae and Pichia | 2007. |
| | | pastoris as secreted | |
| | | enzyme under the alcohol | |
| | | oxidase gene (AOX1) | |
| | | promoter | |
| Lipase | active against broad range of FA | Staphylococcus simulans/ | Sayari, A. et |
| | chain lengths; Asp290Ala | Escherichia coli BL21 | al., 2007. |
| | mutant preference for short FA | (DE3) expressed using a | |
| | esters | pET-14b vector as a His- | |
| | | tagged enzyme | |
| Lipases | LIPY7 and LIPY8 genes | Yarrowia lipolytica/Pichia | Jiang, Z. B. et |
| | | pastoris KM71 cell surface | al., 2007. |
| | | expression as fusion | |
| | | protein with | |
| | | Saccharomyces | |
| | | cerevisiae FLO- | |
| | | flocculation domain | |
| | | sequence, use of whole | |
| | | cell biocatalyst and/or | |
| | | cleaved enzyme | |
| Lipase | lipC gene | Bacillus subtilis ycsK/ | Masayama, A. |
| | | Escherichia coli | et al., 2007. |
| Lipase | optimums 55° C., pH 8.5; stable | Bacillus | Sinchaikul, S. |
| | 30° C. to 65° C.; stable in | stearothermophilus P1/ | et al., 2001. |
| | detergents 0.1% Chaps and/or | Escherichia coli | |
| | Triton X-100 | M15[EP4]; additional | |
| | | expression of site directed | |
| | | Ser-113, Asp-317, and | |
| | | His-358 mutants | |
| | | confirmed active site | |
| | | residues | |
| Lipase | Asp290Ala mutant had altered | Staphylococcus xylosus/ | Mosbah, H. et |
| | FA chain length specificity | Escherichia coli BL21 | al., 2006. |
| | | (DE3) using pET-14b | |
| | | vector, strong T7 | |
| | | promoter, and 6 N- | |
| | | terminal histidines | |
| Lipase | LIP4 mutations A296I, V344Q, | Candida rugosa/Pichia | Lee, L. C. et al., |
| | and V344H improved activity | pastoris | 2007. |
| | against a short chain FA ester; | | |
| | A296I and V344Q mutations | | |
| | improved activity toward a | | |
| | medium and/or a long chain FA | | |
| | ester | | |
| Lipase | preference for C16-C18 a long | Candida rugosa/Pichia | Tang, S. J. et |
| | chain FA ester; stable at 58° C. | pastoris and Escherichia | al., 2001. |
| | when glycosylated in P. pastoris | coli expression improved | |
| | expression; 52° C. unglycosylated | by mutation of 19 non- | |
| | in Escherichia coli expression; | universal CUG codons | |
| | no interfacial activation | into universal codons. | |
| Lipase | Phe94Gly mutant has increased | Rhizomucor miehei/ | Gaskin, D. J. et |
| | preference for a short chain FA | Escherichia coli | al., 2001. |
| | ester | expression of mutants | |
| Lipase | broad substrate specificity, but | Bacillus licheniformis/ | Nthangeni, M. B. |
| | preference for a C6 to C8 FA | Escherichia coli | et al., |
| | ester | expression a secreted | 2001. |
| | | fusion protein with 6 C- | |
| | | terminal histidines. | |
| Lipase | Lysosomal acid | Homo sapiens/ | Ikeda, S. et al., |
| | | Schizosaccharomyces | 2004. |
| | | pombes as secreted | |
| | | protein via feed batch | |
| | | growth | |
| Lipase | Gly311Val mutant stable at | Staphylococcus xylosus/ | Mosbah, H. et |
| | 50° C.; G311D mutant optimum | Escherichia coli BL21 | al., 2007. |
| | pH 6.5; G311K mutant optimum | (DE3) | |
| | pH 9.5 | | |
| Lipase | F417A mutation in neutral lipid | Homo sapiens/ | Alam, M. et al., |
| | binding domain FLXLXXXn | Spodoptera frugiperda | 2006. |
| | reduces ester hydrolysis rate | SF9 cells | |
| Lipase | | Rhizopus oryzae/ | Di Lorenzo, M. |
| | | Escherichia coli | et al., 2005. |
| | | Origami(DE3) using pET- | |
| | | 11d vector expression. | |
| Lipase | LIP1 gene | Candida rugosa/Pichia | Chang, S. W. et |
| | | pastoris | al., 2006^{B}. |
| Lipase | optimums 40° C., pH 5.8 | Malassezia furfur/Pichia | Brunke, S., and |
| | | pastoris | Hube B. et al., |
| | | | 2006. |
| Lipase | optimums 60 to 70° C., pH 8.0 to | Bacillus | Schmidt- |
| | 9.0; stable at pH 9.0 to 11.0; | thermocatenulatus./ | Dannert, C. et |
| | stable in contact with a | Escherichia coli DH5alpha | al., 1996. |
| | detergents and/or an organic | expression via pUC18 | |
| | solvent | vector, Ala replaces 1st | |
| | | Gly of Gly-X-Ser-X-Gly | |
| | | consensus sequence | |
| Lipase | OST gene; 1,3 position | Bacillus sphaericus 205y/ | Sulong, M. R. et |
| | specificity; organic solvent | Escherichia coli | al., 2006. |
| | tolerance; optimums 55° C., pH | | |
| | 7.0 to 8.0; range 5.0 to 13.0 at | | |
| | 37° C.; activity enhance by Ca²⁺, | | |
| | Mg²⁺, dimethylsulfoxide | | |
| | (DMSO), methanol, p-xylene, | | |
| | and/or n-decane | | |
| Lipase | lipB68 gene; optimum 20° C.; a | Pseudomonas fluorescens | Luo, Y. et al., |
| | 1,3 FA ester preference | strain B68/ | 2006. |
| Lipases | LIPY7 and LIPY8 genes | Yarrowia lipolytica/Pichia | Song, H. T. et |
| | | pastoris KM71 secreted | al., 2006. |
| | | expression in the | |
| | | expression vector pPIC9K | |
| | | with 6 × Histidine tag | |
| | | sequence | |
| Lipase | Lip9 gene, stable in contact with | Pseudomonas aeruginosa | Ogino, H. et |
| | an organic solvent | LST-03/Escherichia coli | al., 2007. |
| | | coexpression with lipase- | |
| | | specific foldase (Lif9), T7 | |
| | | promoter used, lipase | |
| | | signal peptide deleted, | |
| | | over expression inclusion | |
| | | bodies refolded | |
| Lipases | lipase A and lipase B | Bacillus subtilis/ | Detry, J. et al., |
| | | Escherichia coli purified or | 2006. |
| | | crude cell lyophilizate | |
| | | preparations by batch and | |
| | | repetitive batch growth. | |
| Lipase | YILip2 gene; optimums 40° C., pH | Yarrowia lipolytica/Pichia | Yu, M et al., |
| | 8.0; preference for a C12 to C16 | pastoris X-33, secretion | 2007. |
| | long chain FA ester | expression as fusion | |
| | | protein with | |
| | | Saccharomyces | |
| | | cerevisiae secretion signal | |
| | | peptide, under methanol | |
| | | inducible promoter of the | |
| | | alcohol oxidase 1 gene in | |
| | | pPICZalphaA vector, fed | |
| | | batch growth | |
| Lipase | | Candida rugosa/Pichia | Chang, S. W. et |
| | | pastoris expression | al., 2006^{C}. |
| | | increased over 4 fold by | |
| | | mutating codons into P. | |
| | | pastoris preferred codons | |
| Lipase/ | vst gene; preference for a C12 | Vibrio harveyi strain AP6/ | Teo, J. W. et |
| Carboxylesterase | long chain FA ester, able to | Escherichia coli TOP10 | al., 2003. |
| | hydrolyze a short, a medium | cell expression as a | |
| | and/or a longer chain FA ester | carboxy-terminal 6 × His | |
| | | tagged enzyme | |
| Lipase/ | broad specificity for a 2C to a | Oil-degrading bacterium, | Mizuguchi, S. |
| Carboxylesterase | 18C FA ester | strain HD-1/Escherichia | et al., 1999. |
| | | coli | |
| Lipases/ | multiple isolates | Lipase/esterase libraries/ | Ahn, J. M. et |
| Carboxylesterases | | Escherichia coli secretion | al., 2004. |
| | | expression | |
| Lipase/ | S-enantioselective; preference | Yarrowia lipolytica CL180/ | Kim, J. T. et al., |
| Carboxylesterase | for <= a 10C FA ester; optimum | Escherichia coli | 2007. |
| | pH 7.5, 35° C. | | |
| Co-lipase | | Homo sapiens/Pichia | D'Silva, S. et |
| | | pastoris | al., 2007. |
| Phospholipase/ | selective for a phospholipid | Arabidopsis rosette/ | Lo, M. et al., |
| Lipase | | Escherichia coli | 2004. |
| Lipases/Cutinase | Bacillus subtilis and Serratia | Bacillus subtilis, Fusarium | Becker, S. et |
| | marcescens lipases, and | solani pisi, Serratia | al., 2005. |
| | cutinase from Fusarium solani | marcescens/Escherichia | |
| | pisi | coli expressed lipolytic on | |
| | | cell surface as a | |
| | | membrane anchored | |
| | | fusion proteins | |
| Lipoprotein lipase | | Homo sapiens/rabbits | Fan, J. et al., |
| | | (transgenic) | 2001. |
| Lipoprotein lipase | multiple mutations to alter | Avian/Chinese hamster | Sendak, R. A., |
| | protein surface charge mildly | ovary cells expression, | and |
| | reduced activity | multiple site-directed | Bensadoun A. J, |
| | | mutations Lys 321, Arg | 1998. |
| | | 405, Arg 407, Lys 409, | |
| | | Lys 415, and Lys 416 for | |
| | | alter heparin-Sepharose | |
| | | binding | |
| Lipoprotein lipase | | Homo sapiens/insect | Zhang, L. et |
| | | cells (sf21) | al., 2003. |
| Acylglycerol lipase | | Mus musculus/African | Karlsson, M. et |
| | | green monkey COS cells | al., 1997. |
| Acylglycerol lipase | | Mus musculus/Sf9 cells | Karlsson, M. et |
| | | via a baculovirus-insect | al., 2000. |
| | | expression system | |
| Acylglycerol lipase | diacylglycerol lipase activity | Penicillium camembertii U- | Yamaguchi, S. |
| | | 150/Aspergillus oryzae, | et al., 1997. |
| | | expressed using own | |
| | | promoter | |
| Acylglycerol lipase | | Bacillus sp. strain H-257/ | Kitaura, S. et |
| | | Escherichia coli via a | al., 2001. |
| | | pACYC184 plasmid vector | |
| Acylglycerol lipase | Rv0183 gene; preference for a | Mycobacterium | Côtes, K. et al., |
| | monoacylglycerol over a di- or a | tuberculosis/Escherichia | 2007. |
| | triacylglycerol; optimum pH 7.7 | coli | |
| | to 9.0 | | |
| Acylglycerol lipase | | Homo sapiens/mice | Coulthard, M. G. |
| | | expression via adenovirus | et al., |
| | | vector | 1996. |
| Acylglycerol lipase/ | rHPLRP2 gene, active pH 5 to | Homo sapiens/Pichia | Eydoux, C. et |
| Galactolipase | 7+ range | pastoris secreted | al., 2007. |
| Phospholipase/ | patatin protein has multi-enzyme | Solanum tuberosum/ | Andrews, D. L. |
| Acylglycerol lipase/ | activity; strong preference for a | Spodoptera frugiperda | et al., 1988. |
| Galactolipase | monacylglycerol over a di- or a | SF9 cells | |
| | tri-acylglycerols | | |
| Hormone Sensitive | | Homo sapiens/ | Contreras, J. A. |
| Lipase | | Spodoptera frugiperda | et al., 1998. |
| | | SF9 cells | |
| Hormone Sensitive | | Mus musculus/THP-1 | Okazaki, H. et |
| Lipase | | macrophage-like cells by | al., 2002. |
| | | adenovirus-mediated gene | |
| | | delivery | |
| Hormone Sensitive | | Rattus norvegicus/ | Kraemer, F. B. |
| Lipase/Sterol | | Escherichia coli | et al., 1993. |
| esterase | | expression of truncated | |
| | | enzyme fusion protein via | |
| | | a pET expression system | |
| Phospholipase A₁ | | Serratia sp. MK1/ | Song, J. K et |
| | | Escherichia coli, | al., 1999. |
| | | expression improved by | |
| | | promoter with lower | |
| | | strength, lower | |
| | | temperature, enriched | |
| | | medium. | |
| Phospholipase A₁ | | Aspergillus oryzae/ | Shiba, Y. et al., |
| | | Saccharomyces | 2001. |
| | | cerevisiae and A. oryzae | |
| Phospholipase A₁ | mPAPLA1alpha and | Homo sapiens (testes)/ | Hiramatsu, T. |
| | mPAPLA1beta, selective for a | Homo sapiens HeLa cells | et al., 2003. |
| | phosphatidic acid | secretion expression for | |
| | | mPA-PLA1alpha, cell | |
| | | membrane association for | |
| | | mPA-PLA1beta | |
| Phospholipase A₁ | dad1 | Arabidopsis/Escherichia | Ishiguro, S. et |
| | | coli and in Arabidopsis as | al., 2001. |
| | | a fusion with green | |
| | | fluorescent protein | |
| Phospholipase A₂ | optimum pH 8 to 10 | Nicotiana tabacum/ | Fujikawa, R. et |
| | | Escherichia coli | al., 2005. |
| | | expression as a | |
| | | thioredoxin fusion protein | |
| | | within cells | |
| Phospholipase A₂ | cytosolic; cPLA₂delta, | Mus musculus/Homo | Ohto, T. et al., |
| | cPLA₂epsilon and cPLA₂zeta | sapiens embryonic kidney | 2005. |
| | genes; Ca²⁺ dependant activity | 293 cells | |
| Phospholipase A₂ | plaA gene; substrates PC and | Aspergillus nidulans/ | Hong, S. et al., |
| | PE | yeast cells expression of | 2005. |
| | | N-truncated enzyme | |
| Phospholipase A₂ | Lipoprotein-associated | Homo sapiens/Pichia | Zhang, F et al., |
| | | pastoris secretion | 2006. |
| | | expression | |
| Phospholipase A₂ | Ca²⁺ activated | Arabidopsis thaliana/ | Mansfeld, J. et |
| | | Escherichia coli | al., 2006. |
| Phospholipase A₂ | Ca⁺² dependent, optimum pH | Drosophila melanogaster/ | Ryu, Y. et al., |
| | 5.0 | Escherichia coli | 2003. |
| Phospholipase A₂ | 3 isoforms expressed | Naja naja sputatrix/ | Armugam, A. |
| | | Escherichia coli | et al., 1997. |
| Phospholipase A₂ | Calcium independent, AXSXG | Mus musculus, Bos | Hiraoka, M. et |
| | catalytic site sequence. | taurus, and Homo sapiens | al., 2002. |
| | | (kidney)/COS-7 cells via | |
| | | pcDNA3 vector, producing | |
| | | carboxyl-terminally tagged | |
| | | proteins | |
| Phospholipase A₂/ | optimum 90° C. | Aeropyrum pernix K1 | Wang, B. et al., |
| Carboxylesterase | | APE2325/Escherichia | 2004. |
| | | coli BL21 (DE3) Codon | |
| | | Plus-RIL | |
| Phospholipase B | | Guinea pig/Monkey | Nauze, M. et |
| | | Kidney COS-7 cells | al., ″2002. |
| | | expressed including | |
| | | mutants identifying serine | |
| | | 399 as functioning in | |
| | | activity and truncation | |
| | | mutants. | |
| Phospholipase C | active at 70° C. +, pH 3.5-6.0 | Bacillus cereus/Bacillus | Durban, M. A. |
| | | subtilis expression via an | et al., 2007. |
| | | acetoin-controlled | |
| | | expression system | |
| Phospholipase C | phosphatidylinositol-specific | Bacillus thuringiensis/ | Kobayashi, T. |
| | | Bacillus brevis 47 | et al., 1996. |
| | | expression system | |
| Phospholipase C | broad specificity for | Bacillus cereus/ | Tan, C. A. et |
| | phospholipids | Escherichia coli via a T7 | al., 1997. |
| | | expression system, | |
| | | refolded form inclusion | |
| | | bodies | |
| Phospholipase C | phosphoinositide-specific | Zea mays/Escherichia | Zhai, S. et al., |
| | | coli | 2005. |
| Phospholipase C | plc gene; stable at 75° C., | Bacillus cereus/Pichia | Seo, K. H., |
| | optimum pH 4.0-5.0 | pastoris secretion | Rhee JI., 2004. |
| | | expression as an alpha- | |
| | | factor secretion signal | |
| | | peptide fusion protein | |
| Phospholipases C | Phosphoinositide-specific | Pisum sativum/ | Venkataraman, G. |
| | | Escherichia coli | et al., 2003. |
| Phosphatidate | Mg²⁺⁻independent, lyso-PA | Saccharomyces | Toke, D. A. et |
| phosphatase | phosphatase and diacylglycerol | cerevisiae/Sf-9 insect | al., 1998. |
| | pyrophosphate phosphatase | cells | |
| | activity | | |
| Lysophospholipase | | Clonorchis sinensis/ | Ma, C. et al., |
| | | Escherichia coli | 2007. |
| Sterol esterase | | Homo sapiens/COS-7 | Zhao, B. et al., |
| | | cell expression | 2005. |
| Sterol esterase | hncCEH gene, hepatic | Rattus norvegicus/mice | Langston, T. B. |
| | | infected with AdCEH | et al., 2005. |
| | | adenovirus vector under | |
| | | Homo sapiens | |
| | | cytomegalovirus promoter, | |
| | | liver cell enzyme | |
| | | expression evaluated | |
| Sterol esterase | | Rattus norvegicus/ | DiPersio, L. P. |
| | | Spodoptera frugiperda | et al., 1992. |
| | | (Sf9) insect cells secretion | |
| | | expression via a | |
| | | Baculovirus transfer vector | |
| | | pVL1392 | |
| Sterol esterase | | Homo sapiens/COS-1 | Ghosh, S., |
| | | and COS-7 cells | 2000. |
| | | expression via expression | |
| | | vector, pcDNA3.1/V5/His- | |
| | | TOPO, | |
| Sterol esterase | CLR1, CRL3 and CRL4 | Candida rugosa/Pichia | Brocca, S. et |
| | isozymes used to make hybrid | pastoris X33 expression of | al., 2003. |
| | enzymes by switching lid | hybrid protein under the | |
| | sequence into CLR1, conferring | he methanol-inducible | |
| | cholesterol esterase activity and | alcohol oxidase promoter | |
| | detergent sensitivity, but no | | |
| | change in chain length | | |
| | preference | | |
| Sterol esterase | | Rattus norvegicus/Hep | Hall, E. et al., |
| | | G2 cells and Chinese | 2001. |
| | | hamster ovary cells via a | |
| | | replication-defective | |
| | | recombinant adenovirus | |
| | | vector | |
| Sterol esterase | ste1 | Melanocarpus albomyces/ | Kontkanen, H. |
| | | Pichia pastoris and T. reesei | et al., 2006. |
| | | under inducible | |
| | | AOX1 promoter, under the | |
| | | inducible cbh1 promoter, | |
| | | respectively | |
| Galactolipase | Vupat1 gene; active on a | Vigna unguiculata/ | Matos, A. R. et |
| | monogalactosyldiacylglycerol, a | Spodoptera frugiperda | al., 2000. |
| | digalactosyldiacylglycerol and/or a | SF9 cells | |
| | sulphoquinovosyldiacylglycerol | | |
| Galactolipase | | Homo sapiens/Pichia | Sias, B. et al., |
| | | pastoris and insect cells | 2004. |
| Galactolipase | | Homo sapiens/Pichia | Sias, B. et al., |
| | | pastoris and insect cells | 2004. |
| Sphingomyelin | | Bacillus cereus/Bacillus | Tamura, H. et |
| phosphodiesterase | | brevis 47 expression as a | al., 1992. |
| | | cell wall signal sequence | |
| | | fusion protein U211 vector | |
| Sphingomyelin | | Homo sapiens/secretion | Lee, C. Y. et al., |
| phosphodiesterase | | expression in Chinese | 2007. |
| | | hamster ovary cells, N- | |
| | | terminal truncations | |
| | | prevented secretion and | |
| | | enzyme activity | |
| Sphingomyelin | | Homo sapiens/COS-7 | Wu, J. et al., |
| phosphodiesterase | | cell expression of | 2005. |
| | | glycosylation mutants | |
| | | demonstrated less activity | |
| Sphingomyelin | | Bacillus cereus/ | Nishiwaki, H. et |
| phosphodiesterase | | Escherichia coli, | al., 2004. |
| | | His151Ala mutant inactive | |
| Sphingomyelin | Sphingomyelin-specific | Pseudomonas sp. strain | Sueyoshi, N. et |
| phosphodiesterase | sphingomyelinase C; able to | TK4/Escherichia coli | al., 2002. |
| | hydrolyze a short FA ester chain | Dhalpha and | |
| | comprising sphingomyelin; | BL21(DE3)pLysS | |
| | optimum pH 8.0, activated by | | |
| | Mn²⁺ | | |
| Phospholipase D | | Homo sapiens/COS-7 | Lehman, N. et |
| | | cells with a myc-(pcDNA)- | al., 2007. |
| | | PLD2 vector | |
| Phospholipase D | | Arabidopsis thaliana/ | Qin, C. et al., |
| | | Escherichia coli | 2006. |
| Phospholipase D | | Streptoverticillium | Ogino, C. et |
| | | cinnamoneum/ | al., 2004. |
| | | Streptomyces lividans via | |
| | | an Escherichia coli shuttle | |
| | | vector-pUC702 | |
| Phospholipase D | | Homo sapiens/COS7 | Di Fulvio, M. et |
| | | cells | al., 2007. |
| Phospholipase D | | Vigna unguiculata L. Walp/ | Ben, Ali Y. et |
| | | Pichia pastoris secretion | al., 2007. |
| | | expression | |
| Ceramidase | | Pseudomonas aeruginosa | Nieuwenhuizen, W. F. |
| | | PA01/Escherichia coli | et al., |
| | | DH5alpha intracellular | 2003. |
| | | expression under lac- | |
| | | promoter, Escherichia coli | |
| | | BL21 intracellular | |
| | | expression under T7- | |
| | | promoter forming | |
| | | refoldable inclusion bodies | |
| | | without signal, | |
| | | Pseudomonas putida | |
| | | extracellular expression | |
| Ceramidase | | Pseudomonas aeruginosa | Okino, N. et al., |
| | | strain AN17/Escherichia | 1999. |
| | | coli intracellular | |
| | | expression | |
| Ceramidase | calcium may alter activity | Pseudomonas/ | Wu, B. X. et al., |
| | | Escherichia coli | 2006. |
| Ceramidase | | Homo sapiens/Homo | Ferlinz, K. et |
| | | sapiens fibroblasts, | al., 2001. |
| | | glycosylation mutants | |
| | | activity not effected | |
| Cutinase | stable at 50° C., pH 7.0 to 9.2 | Fusarium solani pisi/ | Baptista, R. P. |
| | | Escherichia coli WK-6, | et al., 2003. |
| | | adsorption onto 100 nm | |
| | | diameter poly(methyl | |
| | | methacrylate) (PMMA) | |
| | | latex particles' surface | |
| Cutinase | | Fusarium solani pisi/ | Calado, C. R. et |
| | | Saccharomyces | al., 2004. |
| | | cerevisiae SU50 | |
| | | cultivation via batch or | |
| | | fed-batch cultures | |
| Cutinase | | Fusarium solani pisi/ | Calado, C. R. et |
| | | Saccharomyces | al., 2003.; |
| | | cerevisiae SU50 fed-batch | Calado CR, et |
| | | cultivation for secreted | al., 2002. |
| | | enzyme production | |
| Cutinase | | Fusarium solani pisi/ | Kepka, C. et |
| | | Escherichia coli | al., 2005. |
| | | intracellular expression as | |
| | | a typtophan-proline | |
| | | peptide tag fusion protein | |
| Cutinase | | Monilinia fructicola/Pichia | Wang et al., |
| | | pastoris expression as a | 2002. |
| | | His-tagged fusion protein | |

Chemical modification of lipases, particularly the surface of such enzymes, has been used to improve organic solvent solubility, enhance activity, modify enantioselectivity, or a combination thereof. Such functional equivalents may be produced by reactions with a stearic acid, a polyethylene glycol (e.g., bonds to the free amino groups), a pyridoxyl phosphate, a tetranitromethane (sometimes followed by Na₂S₂O₄), a glutaraldehyde (e.g., cross-linking to produce a cross-linked enzyme crystal know as a “CLEC”), a polystyrene, a polyacrylate, (R)-1-phenylethanol in combination with a molecular coating the enzyme's surface with a lipid at the molecular level; molecular coating the enzyme's surface with a lipid and/or a surfactant at the molecular level (e.g., didodecyl N-D-glucono-L-glutamate), forming a non-covalent complex formation with a surfactant (e.g., an ionic surfactant, a non-ionic surfactant), or a combination thereof [see, for example, “Methods in non-aqueous enzymology” (Gupta, M. N., Ed.) p. 85-89, 95 2000; Kurt Faber, “Biotransformations in Organic Chemistry, a Textbook, Third Edition.” pp. 357-376, 1997] For example, coupling a Pseudomonas sp., lipase with a polyethylene glycol improved enzyme solubility in chlorinated hydrocarbons, benzene, and toluene (Okahata, Y. et al., 1995). In another example, molecular coating a Rhizopus sp. lipase with didodecyl N-D-glucono-L-glutamate enhanced activity 100-fold and improved organic solubility, presumably because the surfactant acted as an interface to alter the lid conformation. (Okahata, Y. and Ijiro, K., 1992; Okahata, Y, Ijiro, K., 1988). Production of a Psuedomonas cepacia and Candida rugosa lipase CLECs enhanced stability, and the C. rugosa CLEC has enhanced enantioselectivity for ketoprofen (Lalonde, J. J. et al., 1995; Persichetti, R. A., 1996). The presence of some chemicals may also enhance stability, such as hexanol, which has been described as improving cutinase's stability (In “Engineering of/with Lipases” (F. Xavier Malcata., Ed.) p. 308, 1996). Chemical modification, such as for example, an alkylation of a lysine's amino moiety(s) with pyridoxal phosphate, nitration with tetranitromethane, with or without sodium hydrosulfite, improved enantiomeric selectivity of Candida rugosa lipase (Kurt Faber, “Biotransformations in Organic Chemistry, a Textbook, Third Edition.” Springer-verlag Berlin Heidelberg, pp. 114-115, 1997).

Other modifications that may be used are described herein, particularly in the processing of a biomolecular composition from a cell and/or biological material into a form for incorporation in a material formulation. All such techniques and compositions in the art and as described herein may be used in preparing a biomolecular composition, particularly in preparation of those compositions that comprise an enzyme (e.g., a cell-based particulate material comprising a lipolytic enzyme, a purified lipolytic enzyme, etc.).

2. OPH Functional Equivalents

Recombinant wild-type and mutant forms of the opd gene have been expressed, predominantly in Escherichia coli, for further characterization and analysis. Unless otherwise noted, the various OPH enzymes, whether wild-type or mutants, that act as functional equivalents were prepared using the OPH genes and encoded enzymes first isolated from Pseudomonas diminuta and Flavobacterium spp.

OPH normally binds two atoms of Zn²⁺ per monomer when endogenously expressed. While binding a Zn²⁺, this enzyme may comprise a stable dimeric enzyme, with a thermal temperature of melting (“Tₘ”) of approximately 75° C. and a conformational stability of approximately 40Killocalorie per mole (“kcal/mol”) (Grimsley, J. K. et al., 1997). However, structural analogs have been made wherein a Co²⁺, a Fe²⁺, a Cu²⁺, a Mn²⁺, a Cd²⁺, and/or a Ni²⁺ are bound instead to produce enzymes with altered stability and rates of activity (Omburo, G. A. et al., 1992). For example, a Co²⁺ substituted OPH does possess a reduced conformational stability (−22Kcal/mol). But this reduction in thermal stability may be offset by the improved catalytic activity of a Co²⁺ substituted OPH in degrading various OP compounds. For example, five-fold or greater rates of detoxification of sarin, soman, and VX were measured for a Co²⁺ substituted OPH relative to OPH binding Zn²⁺ (Kolakoski, J. E. et al., 1997). A structural analog of an OPH sequence may be prepared comprising a Zn²⁺, a Co²⁺, a Fe²⁺, a Cu²⁺, a Mn²⁺, a Cd²⁺, a Ni²⁺, or a combination thereof. Generally, changes in the bound metal may be achieved by using cell growth media during cell expression of the enzyme wherein the concentration of a metal present may be defined, and/or removing the bound metal with a chelator (e.g., 1,10-phenanthroline; 8-hydroxyquinoline-5-sulfphonic acid; ethylenediaminetetraacetic acid) to produce an apo-enzyme, followed by reconstitution of a catalytically active enzyme by contact with a selected metal (Omburo, G. A. et al., 1992; Watkins, L. M. et al., 1997a; Watkins, L. M. et al., 1997b). A structural analog of an OPH sequence may be prepared to comprise one metal atom per monomer.

In an additional example, OPH structure analysis has been conducted using NMR (Omburo, G. A. et al., 1993). In a further example, the X-ray crystal structure for OPH has been determined (Benning, M. M. et al., 1994; Benning, M. M. et al., 1995; Vanhooke, J. L. et al., 1996), including the structure of the enzyme while binding a substrate, further identifying residues involved in substrate binding and catalytic activity (Benning, M. M. et al., 2000). From these structure evaluations, the amino acids His55, His57, His201, His 230, Asp301, and the carbamylated lysine, Lys169, have been identified as coordinating the binding of the active site metal. Additionally, the positively charged amino acids His55, His57, His201, His230, His254, and His257 are counter-balanced by the negatively charged amino acids Asp232, Asp233, Asp235, Asp 253, Asp301, and the carbamylated lysine Lys169 at the active site area. A water molecule and amino acids His 55, His57, Lys169, His201, His230, and Asp301 are thought to be involved in direct metal binding. The amino acid Asp301 may aid a nucleophilic attack by a bound hydroxide upon the phosphorus to promote cleavage of an OP compound, while the amino acid His354 may aid the transfer of a proton from the active site to the surrounding liquid in the latter stages of the reaction (Raushel, F. M., 2002). The amino acids His 254 and His257 are not thought to comprise direct metal binding amino acids, but may comprise residues that interact (e.g., a hydrogen bond, a Van der Waal interaction) with each other and other active site residue(s), such as a residue that directly contact a substrate and/or bind a metal atom. In particular, amino acid His254 may interact with the amino acids His230, Asp232, Asp233, and Asp301. Amino acid His257 may comprise a participant in a hydrophobic substrate-binding pocket. The active site pocket comprises various hydrophobic amino acids, Trp131, Phe132, Leu271, Phe306, and Tyr309. These amino acids may aid the binding of a hydrophobic OP compound (Benning, M. M. et al., 1994; Benning, M. M. et al., 1995; Vanhooke, J. L. et al., 1996). Electrostatic interactions may occur between phosphoryl oxygen, when present, and the side chains of Trp131 and His201. Additionally, the side chains of amino acids Trp131, Phe132, and Phe306 are thought to be orientated toward the atom of the cleaved substrate's leaving group that was previously bonded to the phosphorus atom (Watkins, L. M. et al., 1997a).

Substrate binding subsites known as the small subsite, the large subsite, and the leaving group subsite have been identified (Benning, M. M. et al., 2000; Benning, M. M. et al., 1994; Benning, M. M. et al., 1995; Vanhooke, J. L. et al., 1996). The amino acids Gly60, Ile106, Leu303, and Ser308 are thought to comprise the small subsite. The amino acids Cys59 and Ser61 are near the small subsite, but with the side chains thought to be orientated away from the subsite. The amino acids His254, His257, Leu271, and Met317 are thought to comprise the large subsite. The amino acids Trp131, Phe132, Phe306, and Tyr309 are thought to comprise the leaving group subsite, though Leu271 may be considered part of this subsite as well (Watkins, L. M. et al., 1997a). Comparison of this opd product with the encoded sequence of the opdA gene from Agrobacterium radiobacter P230 revealed that the large subsite possessed generally larger residues that affected activity, specifically the amino acids Arg254, Tyr257, and Phe271 (Horne, I. et al., 2002). Few electrostatic interactions are apparent from the X-ray crystal structure of the inhibitor bound by OPH, and hydrophobic interaction(s) and the size of the subsite(s) may affect substrate specificity, including stereospecificity for a stereoisomer, such as a specific enantiomer of an OP compound's chiral chemical moiety (Chen-Goodspeed, M. et al., 2001b).

Using the sequence and structural knowledge of OPH, numerous mutants of OPH comprising a sequence analog have been specifically produced to alter one or more properties relative to a substrate's cleavage rate (k_{cat}) and/or specificity (k_{cat}/Kₘ). Examples of OPH sequence analog mutants include H55C, H57C, C59A, G60A, S61A, I106A, I106G, W131A, W131F, W131K, F132A, F132H, F132Y, L136Y, L140Y, H201C, H230C, H254A, H254R, H2545, H257A, H257L, H257Y, L271A, L271Y, L303A, F306A, F306E, F306H, F306K, F306Y, S308A, S308G, Y309A, M317A, M317H, M317K, M317R, H55C/H57C, H55C/H201C, H55C/H230C, H57C/H201C, H57C/H230C, A80V/S365P, I106A/F132A, I106A/S308A, I106G/F132G, I106G/S308G, F132Y/F306H, F132H/F306H, F132H/F306Y, F132Y/F306Y, F132A/S308A, F132G/S308G, L182S/V310A, H201C/H230C, H254R/H257L, H55C/H57C/H201C, H55C/H57C/H230C, H55C/H201C/H230C, I106A/F132A/H257Y, I106A/F132A/H257W, I106G/F132G/S308G, L130M/H257Y/I274N, H257Y/1274N/S365P, H55C/H57C/H201C/H230C, I106G/F132G/H257Y/S308G, and/or A14T/A80V/L185R/H257Y/I274N (Li, W.-S. et al., 2001; Gopal, S. et al., 2000; Chen-Goodspeed, M. et al., 2001a; Chen-Goodspeed, M. et al., 2001b; Watkins, L. M. et al., 1997a; Watkins, L. M. et al., 1997b; diSioudi, B. et al., 1999; Cho, C. M.-H. et al., 2002; Shim, H. et al., 1996; Raushel, F. M., 2002; Wu, F. et al., 2000a; diSioudi, B. D. et al., 1999).

For example, the sequence and structural information has been used in production of mutants of OPH possessing cysteine substitutions at the metal binding histidines His55, His57, His201, and His230. OPH mutants H55C, H57C, H201C, H230C, H55C/H57C, H55C/H201C, H55C/H230C, H57C/H201C, H57C/H230C, H201C/H230C, H55C/H57C/H201C, H55C/H57C/H230C, H55C/H201C/H230C, H57C/H201C/H230C, and H55C/H57C/H201C/H230C were produced binding either a Zn²⁺; a Co²⁺ and/or a Cd²⁺. The H57C mutant had between 50% (i.e., binding a Cd²⁺, a Zn²⁺) and 200% (i.e., binding a Co²⁺) wild-type OPH activity for paraoxon cleavage. The H201C mutant had about 10% activity, the H230C mutant had less than 1% activity, and the H55C mutant bound one atom of a Co²⁺ and possessed little detectable activity, but may still be useful if possessing an useful property (e.g., enhanced stability) (Watkins, L. M., 1997b).

In an additional example, the sequence and structural information has been used in production of mutants of OPH possessing altered metal binding and/or bond-type cleavage properties. OPH mutants H254R, H257L, and H254R/H257L have been made to alter amino acids that are thought to interact with nearby metal-binding amino acids. These mutants also reduced the number of metal ions (i.e., Co²⁺, Zn²⁺) binding the enzyme dimer from four to two, while still retaining 5% to greater than 100% catalytic rates for the various substrates. These reduced metal mutants possess enhanced specificity for larger substrates such as NPPMP and demeton-S, and reduced specificity for the smaller substrate diisopropyl fluorophosphonate (diSioudi, B. et al., 1999). In a further example, the H254R mutant and the H257L mutant each demonstrated a greater than four-fold increase in catalytic activity and specificity against VX and its analog demeton S. The H257L mutant also demonstrated a five-fold enhanced specificity against soman and its analog NPPMP (diSioudi, B. D. et al., 1999).

In an example, specific mutants of OPH (i.e., a phosphotriesterase), were designed and produced to aid phosphodiester substrates to bind and be cleaved by OPH. These substrates either comprised a negative charge and/or a large amide moiety. A M317A mutant was created to enlarge the size of the large subsite, and M317H, M317K, and M317R mutants were created to incorporate a cationic group in the active site. The M317A mutant demonstrated a 200-fold cleavage rate enhancement in the presence of alkylamines, which were added to reduce the substrate's negative charge. The M317H, M317K, and M317R mutants demonstrated modest improvements in rate and/or specificity, including a 7-fold k_{cat}/Kₘ improvement for the M317K mutant (Shim, H. et al., 1998).

In a further example, the W131K, F132Y, F132H, F306Y, F306H, F306K, F306E, F132H/F306H, F132Y/F306Y, F132Y/F306H, and F132H/F306Y mutants were made to add and/or change the side chain of active site residues to form a hydrogen bond and/or donate a hydrogen to a cleaved substrate's leaving group, to enhance the rate of cleavage for certain substrates, such as phosphofluoridates. The F132Y, F132H, F306Y, F306H, F132H/F306H, F132Y/F306Y, F132Y/F306H, and F132H/F306Y mutants all demonstrated enhanced enzymatic cleavage rates, of about three- to ten-fold improvement, against the phosphonofluoridate, diisopropyl fluorophosphonate (Watkins, L. M. et al., 1997a).

In an additional example, OPH mutants W131F, F132Y, L136Y, L140Y, L271Y and H257L were designed to modify the active site size and placement of amino acid side chains to refine the structure of binding subsites to specifically fit the binding of a VX substrate. The refinement of the active site structure produced a 33% increase in cleavage activity against VX in the L136Y mutant (Gopal, S. et al., 2000).

Various mutants of OPH have been made to alter the steriospecificity, and in some cases, the rate of reaction, by substitutions in substrate binding subsites. For example, the C59A, G60A, S61A, I106A, W131A, F132A, H254A, H257A, L271A, L303A, F306A, S308A, Y309A, and M317A mutants of OPH have been produced to alter the size of various amino acids associated with the small subsite, the large subsite and the leaving group subsite, to alter enzyme activity and selectivity, including stereoselectivity, for various OP compounds. The G60A mutant reduced the size of the small subsite, and decreased both rate (k_{cat}) and specificity (k_{cat}/Kₐ) for Rₚ-enantiomers, thereby enhancing the overall specificity for some Sₚ-enantiomers to over 11,000:1. Mutants I106A and S308A, which enlarged the size of the small subsite, as well as mutant F132A, which enlarged the leaving group subsite, all increased the reaction rates for Rₚ-enantiomers and reduced the specificity for Sₚ-enantiomers (Chen-Goodspeed, M. et al., 2001a).

Additional mutants I106A/F132A, I106A/S308A, F132A/S308A, I106G, F132G, S308G, I106G/F132G, I106G/S308G, F132G/S308G, and I106G/F132G/S308G were produced to further enlarge the small subsite and leaving group subsite. These OPH mutants demonstrated enhanced selectivity for Rₚ-enantiomers. Mutants H254Y, H254F, H257Y, H257F, H257W, H257L, L271Y, L271F, L271W, M317Y, M317F, and M317W were produced to shrink the large subsite, with the H257Y mutant, for example, demonstrating a reduced selectivity for Sₚ-enantiomers (Chen-Goodspeed, M. et al., 2001b). Further mutants I106A/H257Y, F132A/H257Y, I106A/F132A/H257Y, I106A/H257Y/S308A, I106A/F132A/H257W, F132A/H257Y/5308A, I106G/H257Y, F132G/H257Y, I106G/F132G/H257Y, I106G/H257Y/S308G, and I106G/F132G/H257Y/S308G were made to simultaneously enlarge the small subsite and shrink the large subsite. Mutants such as H257Y, I106A/H257Y, I106G, I106A/F132A, and I106G/F132G/S308G were effective in altering steriospecificity for Sₚ:Rₚ enantiomer ratios of some substrates to less than 3:1 ratios. Mutants including F132A/H257Y, I106A/F132A/H257W, I106G/F132G/H257Y, and I106G/F132G/H257Y/S308G demonstrated a reversal of selectivity for Sₚ:Rₚ enantiomer ratios of some substrates to ratios from 3.6:1 to 460:1. In some cases, such a change in steriospecificity was produced by enhancing the rate of catalysis of a Rₚ enantiomer with little change on the rate of Sₚ enantiomer cleavage (Chen-Goodspeed, M. et al., 2001b; Wu, F. et al., 2000a).

Such alterations in stereoselectivity may enhance OPH performance against a specific OP compound that may comprise a target of detoxification, including a CWA. Enlargement of the small subsite by mutations that substitute the Ile106 and Phe132 residues with the less bulky amino acid alanine and/or reduction of the large subsite by a mutation that substitutes His257 with the bulkier amino acid phenylalanine increased catalytic rates for the Sₚ-isomer; and decreased the catalytic rates for the Rₚ-isomers of a sarin analog, thus resulting in a triple mutant, I106A/F132A/H257Y, with a reversed sterioselectivity such as a Sₚ:Rₚ preference of 30:1 for the isomers of the sarin analog. A mutant of OPH designated G60A has also been created with enhanced steriospecificity relative to specific analogs of enantiomers of sarin and soman (Li, W.-S. et al., 2001; Raushel, F. M., 2002). Of greater interest, these mutant forms of OPH have been directly assayed against sarin and soman nerve agents, and demonstrated enhanced detoxification rates for racemic mixtures of sarin or soman enantiomers. Wild-type OPH has a k_{cat} for sarin of 56 s⁻¹, while the I106A/F132A/H257Y mutant has k_{cat} for sarin of 1000 s⁻¹. Additionally, wild-type OPH has a k_{cat} for soman of 5 s⁻¹, while the G60A Mutant has k_{cat} for soman of 10 s⁻¹ (Kolakoski, Jan E. et al. 1997; Li, W.-S. et al., 2001).

It is also possible to produce a mutant enzyme with an enhanced enzymatic property against a specific substrate by evolutionary selection and/or exchange of encoding DNA segments with related proteins rather than rational design. Such techniques may screen hundreds or thousands of mutants for enhanced cleavage rates against a specific substrate [see, for example, “Directed Enzyme Evolution: Screening and Selection Methods (Methods in Molecular Biology) (Arnold, F. H. and Georgiou, G) Humana Press, Totowa, N.J., 2003; Primrose, S. et al., “Principles of Gene Manipulation” pp. 301-303, 2001]. The mutants identified may possess substitutions at amino acids that have not been identified as directly comprising the active site, or its binding subsites, using techniques such as NMR, X-ray crystallography and computer structure analysis, but still contribute to activity for one or more substrates. For example, selection of OPH mutants based upon enhanced cleavage of methyl parathion identified the A80V/S365P, L182SN310A, I274N, H257Y, H257Y/I274N/S365P, L130M/H257Y/I274N, and A14T/A80V/L185R/H257Y/I274N mutants as having enhanced activity. Amino acids Ile274 and Val310 are within 10A of the active site, though not originally identified as part of the active site from X-ray and computer structure analysis. However, mutants with substitutions at these amino acids demonstrated improved activity, with mutants comprising the I274N and H257Y substitutions particularly active against methyl parathion. Additionally, the mutant, A14T/A80V/L185R/H257Y/I274N, further comprising a L185R substitution, was active having a 25-fold improvement against methyl parathion (Cho, C. M.-H. et al., 2002).

In an example, a functional equivalent of OPH may be prepared that lacks the first 29-31 amino acids of the wild-type enzyme. The wild-type form of OPH endogenously or recombinantly expressed in Pseudomonas or Flavobacterium removes the first N-terminal 29 amino acids from the precursor protein to produce the mature, enzymatically active protein (Mulbry, W. and Karns, J., 1989; Serdar, C. M. et al., 1989). Recombinant expressed OPH in Gliocladium virens apparently removes part or all of this sequence (Dave, K. I. et al., 1994b). Recombinant expressed OPH in Streptomyces lividans primarily has the first 29 or 30 amino acids removed during processing, with a few percent of the functional equivalents having the first 31 amino acids removed (Rowland, S. S. et al., 1992). Recombinant expressed OPH in Spodoptera frugiperda cells has the first 30 amino acids removed during processing (Dave, K. I. et al., 1994a).

The 29 amino acid leader peptide sequence targets OPH enzyme to the cell membrane in Escherichia coli, and this sequence may be partly or fully removed during cellular processing (Dave, K. I. et al., 1994a; Miller, C. E., 1992; Serdar, C. M. et al., 1989; Mulbry, W. and Karns, J., 1989). The association of OPH comprising the leader peptide sequence with the cell membrane in Escherichia coli expression systems seems to be relatively weak, as brief 15 second sonication releases most of the activity into the extracellular environment (Dave, K. I. et al., 1994a). For example, recombinant OPH may be expressed without this leader peptide sequence to enhance enzyme stability and expression efficiency in Escherichia coli (Serdar, C. M., et al. 1989). In another example, recombinant expression efficiency in Pseudomonas putida for OPH was improved by retaining this sequence, indicating that different species of bacteria may have varying preferences for a signal sequence (Walker, A. W. and Keasling, J. D., 2002). However, the length of an enzymatic sequence may be readily modified to improve expression or other properties in a particular organism, or select a cell with a relatively good ability to express a biomolecule, in light of the present disclosures and methods in the art (see U.S. Pat. Nos. 6,469,145, 5,589,386 and 5,484,728)

In an example, recombinant OPH sequence-length mutants have been expressed wherein the first 33 amino acids of OPH have been removed, and a peptide sequence M-I-T-N-5 added at the N-terminus (Omburo, G. A. et al., 1992; Mulbry, W. and Karns, J., 1989). Often removal of the 29 amino acid sequence may be used when expressing mutants of OPH comprising one or more amino acid substitutions such as the C59A, G60A, S61A, I106A, W131A, F132A, H254A, H257A, L271A, L303A, F306A, S308A, Y309A, M317A, I106A/F132A, I106A/S308A, F132A/S308A, I106G, F132G, S308G, I106G/F132G, I106G/S308G, F132G/S308G, I106G/F132G/S308G, H254Y, H254F, H257Y, H257F, H257W, H257L, L271Y, L271W, M317Y, M317F, M317W, I106A/H257Y, F132A/H257Y, I106A/F132A/H257Y, I106A/H257Y/5308A, I106A/F132A/H257W, F132A/H257Y/S308A, I106G/H257Y, F132G/H257Y, I106G/F132G/H257Y, I106G/H257Y/S308G, and I106G/F132G/H257Y/5308G mutants (Chen-Goodspeed, M. et al., 2001a). In a further example, LacZ-OPH fusion protein mutants lacking the 29 amino acid leader peptide sequence and comprising an amino acid substitution mutant such as W131F, F132Y, L136Y, L140Y, H257L, L271L, L271Y, F306A, or F306Y have been recombinantly expressed (Gopal, S. et al., 2000).

In an additional example, OPH mutants that comprise additional amino acid sequences are also known in the art. An OPH fusion protein lacking the 29 amino acid leader sequence and possessing an additional C-terminal flag octapeptide sequence was expressed and localized in the cytoplasm of Escherichia coli (Wang, J. et al., 2001). In another example, nucleic acids encoding truncated versions of the ice nucleation protein (“InaV”) from Pseudomonas syringae have been used to construct vectors that express OPH-InaV fusion proteins in Escherichia coli. The InaV sequences targeted and anchored the OPH-InaV fusion proteins to the cells' outer membrane (Shimazu, M. et al., 2001a; Wang, A. A. et al., 2002). In a further example, a vector encoding a similar fusion protein was expressed in Moraxella sp., and demonstrated a 70-fold improved OPH activity on the cell surface compared to Escherichia coli expression (Shimazu, M. et al., 2001b). In a further example, fusion proteins comprising the signal sequence and first nine amino acids of lipoprotein, a transmembrane domain of outer membrane protein A (“Lpp-OmpA”), and either a wild-type OPH sequence or an OPH truncation mutant lacking the first 29 amino acids has been expressed in Escherichia coli. These OPH-Lpp-OmpA fusion proteins were targeted and anchored to the Escherichia coli cell membrane, though the OPH truncation mutant had 5% to 10% the activity of the wild-type OPH sequence (Richins, R. D. et al., 1997; Kaneva, I. et al., 1998). In one example, a fusion protein comprising N-terminus to C-terminus, a (His)6 polyhistidine tag, a green fluorescent protein (“GFP”), an enterokinase recognition site, and an OPH sequence lacking the 29 amino acid leader sequence has been expressed within Escherichia coli cells (Wu, C.-F. et al., 2000b, Wu, C.-F. et al., 2002). A similar fusion protein a (His)6 polyhistidine tag, an enterokinase recognition site, and an OPH sequence lacking the 29 amino acid leader sequence has also been expressed within Escherichia coli cells (Wu, C.-F. et al., 2002). Additionally, variations of these GFP-OPH fusion proteins have been expressed within Escherichia coli cells where a second enterokinase recognition site was placed at the C-terminus of the OPH gene fragment sequence, followed by a second OPH gene fragment sequence (Wu, C.-F. et al., 2001b). The GFP sequence produced fluorescence that was proportional to both the quantity of the fusion protein, and the activity of the OPH sequence, providing a fluorescent assay of enzyme activity and stability in GFP-OPH fusion proteins (Wu, C.-F. et al., 2000b, Wu, C.-F. et al., 2002).

In a further example, a fusion protein comprising an elastin-like polypeptide (“ELP”) sequence, a polyglycine linker sequence, and an OPH sequence was expressed in Escherichia coli (Shimazu, M. et al., 2002). In an additional example, a cellulose-binding domain at the N-terminus of an OPH fusion protein lacking the 29 amino acid leader sequence, and a similar fusion protein wherein OPH possessed the leader sequence, where both predominantly excreted into the external medium as soluble proteins by recombinant expression in Escherichia coli (Richins, R. D. et al., 2000).

3. Paraoxonase Functional Equivalents

Various chemical modifications to the amino acid residues of the recombinantly expressed human paraoxonase have been used to identify specific residues including tryptophans, histidines, aspartic acids, and glutamic acids as functioning in enzymatic activity for the cleavage of phenylacetate, paraoxon, chlorpyrifosoxon, and diazoxon. Additionally, comparison to conserved residues in human, mouse, rabbit, rat dog, chicken, and turkey paraoxonase enzymes was used to further identify amino acids for the production of specific mutants. Site-directed mutagenesis was used to alter the enzymatic activity of human paraoxonase through conservative and non-conservative substitutions, and thus clarify the specific amino acids functioning in enzymatic activity. Specific paraoxonase mutants include the sequence analogs E32A, E48A, E52A, D53A, D88A, D107A, H114N, D121A, H133N, H154N, H160N, W193A, W193F, W201A, W201F, H242N, H245N, H250N, W253A, W253F, D273A, W280A, W280F, H284N, and/or H347N.

The various paraoxonase mutants generally had different enzymatic properties. For example, W253A had a 2-fold greater k_{cat}; and W201F, W253A and W253F each had a 2 to 4 fold increase in k_{cat}, though W201F also had a lower substrate affinity. A non-conservative substitution mutant W280A had 1% wild-type paraoxonase activity, but the conservative substitution mutant W280F had similar activity as the wild-type paraoxonase (Josse, D. et al., 1999; Josse, D. et al., 2001).

4. Squid-Type DFPase Functional Equivalents

Various chemical modifications to the amino acid residues of the recombinantly expressed squid-type DFPase from Loligo vulgaris has been used to identify which specific types of residues of modified arginines, aspartates, cysteines, glutamates, histidines, lysines, and tyrosines, function in enzymatic activity for the cleavage of DFP. Modification of histidines generally reduced enzyme activity, and site-directed mutagenesis was used to clarify which specific histidines function in enzymatic activity. Specific squid-type DFPase mutants include the sequence analogs H181N, H224N, H274N, H219N, H248N, and/or H287N.

The H287N mutant lost about 96% activity, and may act as a hydrogen acceptor in active site reactions. The H181N and H274N mutants lost between 15% and 19% activity, and are thought to help stabilize the enzyme. The H224N mutant gained about 14% activity, indicating that alterations to this residue may also affect activity (Hartleib, J. and Ruterjans, H., 2001b).

In a further example of squid-type DFPase functional equivalents, recombinant squid-type DFPase sequence-length mutants have been expressed wherein a (His)6 tag sequence and a thrombin cleavage site has been added to the squid-type DFPase (Hartleib, J. and Ruterjans, H., 2001a). In an additional example, a polypeptide comprising amino acids 1-148 of squid-type DFPase has been admixed with a polypeptide comprising amino acids 149-314 of squid-type DFPase to produce an active enzyme (Hartleib, J. and Ruterjans, H., 2001a).

### F. COMBINATIONS OF BIOMOLECULES

In various embodiments, a composition, an article, a method, etc. may comprise one or more selected biomolecules, in various combinations thereof, with a proteinaceous molecule (e.g., an enzyme, a peptide that binds a ligand, a polypeptide that binds a ligand, an antimicrobial peptide, an antifouling peptide) being a type of biomolecule in certain facets. For example, any combination of biomolecules, such as an enzyme (e.g., an antimicrobial enzyme, organophosphorous compound degrading enzyme, an esterase, a peptidase, a lipolytic enzyme, an antifouling enzyme, etc) and/or a peptide (e.g., an antimicrobial peptide, an antifouling enzyme) described herein are contemplated for incorporation into a material formulation (e.g., a surface treatment, a filler, a biomolecular composition), and may be used to confer one or more properties (e.g., one or more enzyme activities, one or more binding activities, one or more antimicrobial activities, etc) to such compositions. In specific embodiments, a composition may comprise an endogenous, recombinant, biologically manufactured, chemically synthesized, and/or chemically modified, biomolecule. For example, such a composition may comprises a wild-type enzyme, a recombinant enzyme, a biologically manufactured peptide and/or polypeptide (e.g., a biologically produced enzyme that may be subsequently chemically modified), a chemically synthesized peptide and/or polypeptide, or a combination thereof. In specific aspects, a recombinant proteinaceous molecule comprises a wild-type proteinaceous molecule, a functional equivalent proteinaceous molecule, or a combination thereof. Numerous examples of a biomolecule (e.g., a proteinaceous molecule) with different properties are described herein, and any such biomolecule in the art is contemplated for inclusion in a composition, an article, a method, etc.

A combination of biomolecules may be selected for inclusion in a material formulation, to improve one or more properties of such a composition. Thus, a composition may comprise 1 to 1000 or more different selected biomolecules of interest. For example, as various enzymes have differing binding properties, catalytic properties, stability properties, properties related to environmental safety, etc, one may select a combination of enzymes to confer an expanded range of properties to a composition. In a specific example, a plurality of lipolytic enzymes, with differing abilities to cleave the lipid substrates, may be admixed to confer a larger range of catalytic properties to a composition than achievable by the selection of a single lipolytic enzyme. In a specific example, a material formulation may comprise a plurality of biomolecular compositions. In an additional specific example, one or more layers of a multicoat system comprise one or more different biomolecular compositions to confer differing properties between one layer and at least a second layer of the multicoat system.

In another example, a multifunctional surface treatment (e.g., a paint, a coating) may comprise a combination of biomolecular compositions, such as an OP degrading agent and/or enzyme (see, for example, copending U.S. patent application Ser. No. 10/655,435 filed Sep. 4, 2003 and U.S. patent application Ser. No. 10/792,516 filed Mar. 3, 2004) and/or a cellular material comprising such an activity and one or more antifungal and/or antibacterial peptide(s) (e.g., SEQ ID Nos. 6, 7, 8, 9, 10, 41). Such a surface treatment may provide functions upon application to a surface such as, for example, lend antifungal and anti-bacterial properties to the surface; avoid the problem human toxicity that may be associated with a conventional biocidel compound in a coating (e.g., a paint); usefulness in hospital environments and other health care settings (e.g., deter food poisoning, hospital acquired infections by antibiotic-resistant “super bugs,” deter SARS-like outbreaks); reduce the contamination of a public facility and/or a surface by a toxic chemical (e.g., an OP compound) due to an accidental spill, an improper application of certain insecticide, and/or as a result of deliberate criminal and/or terroristic act; or a combination thereof.

In some embodiments, the concentration of any individual selected biomolecule (e.g., an enzyme, a peptide, a polypeptide) of a material formulation (e.g., the wet weight of a biomolecular composition, the dry weight of a biomolecular composition, the average content in the primary particles of a biomolecular composition, such as the primary particles of a cell-based particulate material) comprises about 0.000000001% to about 100%, of the material formulation. For example, a cell-based particulate material may function as a filler, and may comprise up to about 80% of the volume of material formulation (e.g., a coating, a surface treatment), in some embodiments. In another example, an antibiological peptide may comprise about 0.000000001% to about 20%, 10%, or 5% of a material formulation.

### G. RECOMBINANTLY PRODUCED PROTEINACEOUS MOLECULES

In certain aspects, a proteinaceous molecule may be biologically produced in a cell, a tissue and/or an organism transformed with a genetic expression vector. As used herein, an “expression vector” refers to a carrier nucleic acid molecule, into which a nucleic acid sequence may be inserted, wherein the nucleic acid sequence may be capable of being transcribed into a ribonucleic acid (“RNA”) molecule after introduction into a cell. Usually an expression vector comprises deoxyribonucleic acid (“DNA”). As used herein, an “expression system” refers to an expression vector, and may further comprise additional reagents to promote insertion of a nucleic acid sequence, introduction into a cell, transcription and/or translation. As used herein, a “vector,” refers to a carrier nucleic acid molecule into which a nucleic acid sequence may be inserted for introduction into a cell. Certain vectors are capable of replication of the vector and/or any inserted nucleic acid sequence in a cell. For example, a viral vector may be used in conjunction with either an eukaryotic and/or a prokaryotic host cell, particularly one permissive for replication and/or expression of the vector. A cell capable of being transformed with a vector may be known herein as a “host cell.”

In general embodiments, the inserted nucleic acid sequence encodes for at least part of a gene product. In some embodiments wherein the nucleic acid sequence may be transcribed into a RNA molecule, the RNA molecule may be then translated into a proteinaceous molecule. As used herein, a “gene” refers to a nucleic acid sequence isolated from an organism, and/or man-made copies or mutants thereof, comprising a nucleic acid sequence capable of being transcribed and/or translated in an organism. A “gene product” comprises the transcribed RNA and/or translated proteinaceous molecule from a gene. Often, partial nucleic acid sequences of a gene, known herein as a “gene fragment,” are used to produce a part of the gene product. Many gene and gene fragment sequences are known in the art, and are both commercially available and/or publicly disclosed at a database such as Genbank. A gene and/or a gene fragment may be used to recombinantly produce a proteinaceous molecule and/or in construction of a fusion protein comprising a proteinaceous molecule.

In certain embodiments, a nucleic acid sequence such as a nucleic acid sequence encoding an enzyme, and/or any other desired RNA and/or proteinaceous molecule (as well as a nucleic acid sequence comprising a promoter, a ribosome binding site, an enhancer, a transcription terminator, an origin of replication, and/or other nucleic acid sequences, including but not limited to those described herein may be recombinantly produced and/or synthesized using any method or technique in the art in various combinations. [In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002; In “Current Protocols in Cell Biology” (Morgan, K. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Pharmacology” (Taylor, G. Ed.) John Wiley & Sons, 2002; In “Current Protocols in Cytometry” (Robinson, J. P. Ed.) John Wiley & Sons, 2002; In “Current Protocols in Immunology” (Coico, R. Ed.) John Wiley & Sons, 2002]. For example, a gene and/or a gene fragment encoding an enzyme of interest may be isolated and/or amplified through polymerase chain reaction (“PCR™”) technology. Often such nucleic acid sequence may be readily available from a public database and/or a commercial vendor, as previously described.

Nucleic acid sequences, called codons, encoding for each amino acid are used to copy and/or mutate a nucleic acid sequence to produce a desired mutant in an expressed amino acid sequence. Codons comprise nucleotides such as adenine (“A”), cytosine (“C”), guanine (“G”), thymine (“T”) and uracil (“U”).

The common amino acids are generally encoded by the following codons: alanine by GCU, GCC, GCA, or GCG; arginine by CGU, CGC, CGA, CGG, AGA, or AGG; aspartic acid by GAU or GAC; asparagine by AAU or AAC; cysteine by UGU or UGC; glutamic acid by GAA or GAG; glutamine by CAA or CAG; glycine by GGU, GGC, GGA, or GGG; histidine by CAU or CAC; isoleucine by AUU, AUC, or AUA; leucine by UUA, UUG, CUU, CUC, CUA, or CUG; lysine by AAA or AAG; methionine by AUG; phenylalanine by UUU or UUC; proline by CCU, CCC, CCA, or CCG; serine by AGU, AGC, UCU, UCC, UCA, or UCG; threonine by ACU, ACC, ACA, or ACG; tryptophan by UGG; tyrosine by UAU or UAC; and valine by GUU, GUC, GUA, or GUG.

A mutation in a nucleic acid encoding a proteinaceous molecule may be introduced into the nucleic acid sequence through any technique in the art. Such a mutation may be bioengineered to a specific region of a nucleic acid comprising one or more codons using a technique such as site-directed mutagenesis and/or cassette mutagenesis. Numerous examples of phosphoric triester hydrolase mutants have been produced using site-directed mutagenesis or cassette mutagenesis, and are described herein, as well as other enzymes.

For recombinant expression, the choice of codons may be made to mimic the host cell's molecular biological activity, to improve the efficiency of expression from an expression vector. For example, codons may be selected to match the preferred codons used by a host cell in expressing endogenous proteins. In some aspects, the codons selected may be chosen to approximate the G-C content of an expressed gene and/or a gene fragment in a host cell's genome, or the G-C content of the genome itself. In other aspects, a host cell may be genetically altered to recognize more efficiently use a variety of codons, such as Escherichia coli host cells that are dna Y gene positive (Brinkmann, U. et al., 1989).

1. General Expression Vector Components and Use

An expression vector may comprise specific nucleic acid sequences such as a promoter, a ribosome binding site, an enhancer, a transcription terminator, an origin of replication, and/or other nucleic acid sequence, including but not limited to those described herein, in various combinations. A nucleic acid sequence may be “exogenous” when foreign to the cell into which the vector is being introduced and/or that the sequence is homologous to a sequence in the cell, but in a position within the host cell nucleic acid in which the sequence is ordinarily not found. An expression vector may have one or more nucleic acid sequences removed by restriction enzyme digestion, modified by mutagenesis, and/or replaced with another more appropriate nucleic acid sequence, for transcription and/or translation in a host cell suitable for the expression vector selected.

A vector may be constructed by recombinant techniques in the art. Further, a vector may be expressed and/or transcribe a nucleic acid sequence and/or translate its cognate proteinaceous molecule. The conditions under which to incubate any of the above described host cells to maintain them and to permit replication of a vector, and techniques and conditions allowing large-scale production of a vector, as well as production of a nucleic acid sequence encoded by a vector into a RNA molecule and/or translation of the RNA molecule into a cognate proteinaceous molecule, may be used.

In certain embodiments, a cell may express multiple gene and/or gene fragment products from the same vector, and/or express more than one vector. Often this occurs simply as part of the normal function of a multi-vector expression system. For example, one gene or gene fragment may be used to produce a repressor that suppresses the activity of a promoter that controls the expression of a gene or a gene fragment of interest. The repressor gene and the desired gene may be on different vectors. However, multiple gene, gene fragment and/or expression systems may be used to express an enzymatic sequence of interest and another gene or gene fragment that may be desired for a particular function. In an example, recombinant Pseudomonas putida has co-expressed OPH from one vector, and the multigenes encoding the enzymes for converting p-nitrophenol to β-ketoadipate from a different vector. The expressed OPH catalyzed the cleavage of parathion to p-nitrophenol. The additionally expressed recombinant enzymes converted the p-nitrophenol, a moderately toxic compound, to β-ketoadipate, thereby detoxifying both an OP compound and the byproducts of its hydrolysis (Walker, A. W. and Keasling, J. D., 2002). In a further example, Escherichia coli cells expressed a cell surface targeted INPNC-OPH fusion protein from one vector to detoxify OP compounds, and co-expressed from a different vector a cell surface targeted Lpp-OmpA-cellulose binding domain fusion protein to immobilize the cell to a cellulose support (Wang, A. A. et al., 2002). In an additional example, a vector co-expressed an antisense RNA sequence to the transcribed stress response gene σ³² and OPH in Escherichia coli. The antisense σ³² RNA was used to reduce the cell's stress response, including proteolytic damage, to an expressed recombinant proteinaceous molecule. A six-fold enhanced specific activity of expressed OPH enzyme was seen (Srivastava, R. et al., 2000). In a further example, multiple OPH fusion proteins were expressed from the same vector using the same promoter but separate ribosome binding sites (Wu, C.-F. et al., 2001b).

An expression vector generally comprises a plurality of functional nucleic acid sequences that either comprise a nucleic acid sequence with a molecular biological function in a host cell, such as a promoter, an enhancer, a ribosome binding site, a transcription terminator, etc, and/or encode a proteinaceous sequence, such as a leader peptide, a polypeptide sequence with enzymatic activity, a peptide and/or a polypeptide with a binding property, etc. A nucleic acid sequence may comprise a “control sequence,” which refers to a nucleic acid sequence that functions in the transcription and possibly translation of an operatively linked coding sequence in a particular host cell. As used herein, an “operatively linked” or “operatively positioned” nucleic acid sequence refers to the placement of one nucleic acid sequence into a functional relationship with another nucleic acid sequence. Vectors and expression vectors may further comprise one or more nucleic acid sequences that serve other functions as well and are described herein.

The various functional nucleic acid sequences that comprise an expression vector are operatively linked so to position the different nucleic acid sequences for function in a host cell. In certain cases, the functional nucleic acid sequences may be contiguous such as placement of a nucleic acid sequence encoding a leader peptide sequence in correct amino acid frame with a nucleic acid sequence encoding a polypeptide comprising a polypeptide sequence with enzymatic activity. In other cases, the functional nucleic acid sequences may be non-contiguous such as placing a nucleic acid sequence comprising an enhancer distal to a nucleic acid sequence comprising such sequences as a promoter, an encoded proteinaceous molecule, a transcription termination sequence, etc. One or more nucleic acid sequences may be operatively linked using methods in the art, particularly ligation at restriction sites that may pre-exist in a nucleic acid sequence and/or be added through mutagenesis.

A “promoter” comprises a control sequence comprising a region of a nucleic acid sequence at which initiation and rate of transcription are controlled. In the context of a nucleic acid sequence comprising a promoter and an additional nucleic acid sequence, particularly one encoding a gene and/or a gene fragment's product, the phrases “operatively linked,” “operatively positioned,” “under control,” and “under transcriptional control” mean that a promoter is in a functional location and/or an orientation in relation to the additional nucleic acid sequence to control transcriptional initiation and/or expression of the additional nucleic acid sequence. A promoter may comprise genetic element(s) at which regulatory protein(s) and molecule(s) may bind such as an RNA polymerase and other transcription factor(s). A promoter employed may be constitutive, tissue-specific, inducible, and/or useful under the appropriate conditions to direct high level expression of the introduced nucleic acid sequence, such as the large-scale production of a recombinant proteinaceous molecule. Examples of a promoter include a lac, a tac, an amp, a heat shock promoter of a P-element of Drosophila, a baculovirus polyhedron gene promoter, or a combination thereof. In a specific example, the nucleic acids encoding OPH have been expressed using the polyhedron promoter of a baculoviral expression vector (Dumas, D. P. et al., 1990). In a further example, a Cochliobolus heterostrophus promoter, prom1, has been used to express a nucleic acid encoding OPH (Dave, K. I. et al., 1994b).

The promoter may be endogenous or heterologous. An “endogenous promoter” comprises one naturally associated with a gene and/or a sequence, as may be obtained by isolating the 5′ non-coding sequences located upstream of the coding segment and/or an exon. Alternatively, the coding nucleic acid sequence may be positioned under the control of a “heterologous promoter” or “recombinant promoter,” which refers to a promoter that may be not normally associated with a nucleic acid sequence in its natural environment.

A specific initiation signal also may be required for efficient translation of a coding sequence by the host cell. Such a signal may include an ATG initiation codon (“start codon”) and/or an adjacent sequence.

Exogenous translational control signals, including the ATG initiation codon, may be provided. Techniques of the art may be used for determining this and providing the signals. The initiation codon may be “in-frame” with the reading frame of the desired coding sequence to ensure translation of the entire insert. The exogenous translational control signal and/or an initiation codon may be either natural or synthetic. The efficiency of expression may be enhanced by the inclusion of an appropriate transcription enhancer.

A promoter may or may not be used in conjunction with an “enhancer,” which refers to a cis-acting regulatory sequence involved in the transcriptional activation of a nucleic acid sequence. An enhancer may comprise one naturally associated with a nucleic acid sequence, located either downstream and/or upstream of that sequence. A recombinant or heterologous enhancer refers also to an enhancer not normally associated with a nucleic acid sequence in its natural environment. Such a promoter and/or enhancer may include a promoter and/or enhancer of another gene, a promoter and/or enhancer isolated from any other prokaryotic, viral, or eukaryotic cell, a promoter and/or enhancer not “naturally occurring,” i.e., a promoter and/or enhancer comprising different elements of different transcriptional regulatory regions, and/or mutations that alter expression. In addition to producing a nucleic acid sequence comprising a promoter and/or enhancer synthetically, a sequence may be produced using recombinant cloning and/or nucleic acid amplification technology, including PCR™, in connection with the compositions disclosed herein (U.S. Pat. No. 4,683,202, U.S. Pat. No. 5,928,906).

A promoter and/or an enhancer that effectively directs the expression of the nucleic acid sequence in the cell type may be chosen for expression. The art of molecular biology generally knows the use of promoters, enhancers, and cell type combinations for expression. Furthermore, the control sequences that direct transcription and/or expression of sequences within non-nuclear organelles, including eukaryotic organelles such as mitochondria, chloroplasts, and the like, may be employed as well.

Vectors may comprise a multiple cloning site (“MCS”), which comprises a nucleic acid region that comprises multiple restriction enzyme sites, any of which may be used in conjunction with standard recombinant technology to digest the vector. “Restriction enzyme digestion” refers to catalytic cleavage of a nucleic acid molecule with an enzyme which functions at specific locations in a nucleic acid molecule. Many of these restriction enzymes are commercially available. Use of such enzymes may be done in accordance with the art. Frequently, a vector may be linearized and/or fragmented using a restriction enzyme that cuts within the MCS to enable an exogenous nucleic acid sequence to be ligated to the vector. “Ligation” refers to the process of forming phosphodiester bonds between two nucleic acid fragments, which may or may not be contiguous with each other. Techniques involving restriction enzymes and ligation reactions in the art of recombinant technology may be applied.

A “fusion protein,” as used herein, comprises an expressed contiguous amino acid sequence comprising a proteinaceous molecule of interest and one or more additional peptide and/or polypeptide sequences. The additional peptide and/or polypeptide sequence generally provides an useful additional property to the fusion protein, including but not limited to, targeting the fusion protein to a particular location within and/or external to the host cell (e.g., a signal peptide); promoting the ease of purification and/or detection of the fusion protein (e.g., a tag, a fusion partner); promoting the ease of removal of one or more additional sequences from the peptide and/or the polypeptide of interest (e.g., a protease cleavage site); and separating one or more sequences of the fusion protein to allow improved activity and/or function of the sequence(s) (e.g., a linker sequence).

As used herein a “tag” comprises a peptide sequence operatively associated to the sequence of another peptide and/or polypeptide sequence. Examples of a tag include a His-tag, a strep-tag, a flag-tag, a T7-tag, a S-tag, a HSV-tag, a polyarginine-tag, a polycysteine-tag, a polyaspartic acid-tag, a polyphenylalanine-tag, or a combination thereof. A His-tag may comprise about 6 to about 10 amino acids in length, and can be incorporated at the N-terminus, C-terminus, and/or within an amino acid sequence for use in detection and purification. A His tag binds affinity columns comprising nickel, and may be eluted using low pH conditions or with imidazole as a competitor (Unger, T. F., 1997). A strep-tag may comprise about 10 amino acids in length, and may be incorporated at the C-terminus. A strep-tag binds streptavidin or affinity resins that comprise streptavidin. A flag-tag may comprise about 8 amino acids in length, and may be incorporated at the N-terminus and/or the C-terminus of an amino acid sequence for use in purification. A T7-tag may comprise about 11 to about 16 amino acids in length, and may be incorporated at the N-terminus and/or within an amino acid sequence for use in purification. A S-tag may comprise about 15 amino acids in length, and may be incorporated at the N-terminus, C-terminus and/or within an amino acid sequence for use in detection and purification. A HSV-tag may comprise about 11 amino acids in length, and may be incorporated at the C-terminus of an amino acid sequence for use in purification. The HSV tag binds an anti-HSV antibody in purification procedures (Unger, T. F., 1997). A polyarginine-tag may comprise about 5 to about 15 amino acids in length, and may be incorporated at the C-terminus of an amino acid sequence for use in purification. A polycysteine-tag may comprise about 4 amino acids in length, and may be incorporated at the N-terminus of an amino acid sequence for use in purification. A polyaspartic acid-tag may comprise about 5 to about 16 amino acids in length, and may be incorporated at the C-terminus of an amino acid sequence for use in purification. A polyphenylalanine-tag may comprise about 11 amino acids in length, and may be incorporated at the N-terminus of an amino acid sequence for use in purification.

In one example, a (His)6 tag sequence has been used to purify fusion proteins comprising GFP-OPH or OPH using immobilized metal affinity chromatography (“IMAC”) (Wu, C.-F. et al., 2000b; Wu, C.-F. et al., 2002). In a further example, a (His)6 tag sequence followed by a thrombin cleavage site has been used to purify fusion proteins comprising squid-type DFPase using IMAC (Hartleib, J. and Ruterjans, H., 2001a). In a further example, an OPH fusion protein comprising a C-terminal flag has been expressed (Wang, J. et al., 2001).

As used herein a “fusion partner” comprises a polypeptide operatively associated to the sequence of another peptide and/or polypeptide of interest. Properties that a fusion partner may confer to a fusion protein include, but are not limited to, enhanced expression, enhanced solubility, ease of detection, and/or ease of purification of a fusion protein. Examples of a fusion partner include a thioredoxin, a cellulose-binding domain, a calmodulin binding domain, an avidin, a protein A, a protein G, a glutathione-5-transferase, a chitin-binding domain, an ELP, a maltose-binding domain, or a combination thereof. Thioredoxin may be incorporated at the N-terminus and/or the C-terminus of an amino acid sequence for use in purification. A cellulose-binding domain binds a variety of resins comprising cellulose or chitin (Unger, T. F., 1997). A calmodulin-binding domain binds affinity resins comprising calmodulin in the presence of calcium, and allows elution of the fusion protein in the presence of ethylene glycol tetra acetic acid (“EGTA”) (Unger, T. F., 1997). Avidin may be useful in purification and/or detection. A protein A and/or a protein G binds a variety of anti-bodies for ease of purification. Protein A may be bound to an IgG sepharose resin (Unger, T. F., 1997). Streptavidin may be useful in purification and/or detection. Glutathione-5-transferase may be incorporated at the N-terminus of an amino acid sequence for use in detection and/or purification. Glutathione-5-transferase binds affinity resins comprising glutathione (Unger, T. F., 1997). An elastin-like polypeptide comprises repeating sequences (e.g., 78 repeats) which reversibly converts itself, and thus the fusion protein, from an aqueous soluble polypeptide to an insoluble polypeptide above an empirically determined transition temperature. The transition temperature may be affected by the number of repeats, and may be determined spectrographically using techniques known in the art, including measurements at 655 nano meters (“nm”) over a 4° C. to 80° C. range (Urry, D. W. 1992; Shimazu, M. et al., 2002). A chitin-binding domain comprises an intein cleavage site sequence, and may be incorporated at the C-terminus for purification. The chitin-binding domain binds affinity resins comprising chitin, and an intein cleavage site sequence allows the self-cleavage in the presence of thiols at reduced temperature to release the peptide and/or the polypeptide sequence of interest (Unger, T. F., 1997). A maltose-binding domain may be incorporated at the N-terminus and/or the C-terminus of an amino acid sequence for use in detection and/or purification. A maltose-binding domain sequence usually further comprises a ten amino acid poly asparagine sequence between the maltose binding domain and the sequence of interest to aid the maltose-binding domain in binding affinity resins comprising amylose (Unger, T. F., 1997).

In an example, a fusion protein comprising an elastin-like polypeptide sequence and an OPH sequence has been expressed (Shimazu, M. et al., 2002). In a further example, a cellulose-binding domain-OPH fusion protein has also been recombinantly expressed (Richins, R. D. et al., 2000). In an additional example, a maltose binding protein-E3 carboxylesterase fusion protein has been recombinantly expressed (Claudianos, C. et al., 1999)

A protease cleavage site promotes proteolytic removal of the fusion partner from the peptide and/or the polypeptide of interest. A fusion protein may be bound to an affinity resin, and cleavage at the cleavage site promotes the ease of purification of a peptide and/or a polypeptide of interest with much (e.g., most) to about all of the tag and/or the fusion partner sequence removed (Unger, T. F., 1997). Examples of protease cleavage sites used in the art include the factor Xa cleavage site, which comprises about four amino acids in length; the enterokinase cleavage site, which comprises about five amino acids in length; the thrombin cleavage site, which comprises about six amino acids in length; the rTEV protease cleavage site, which comprises about seven amino acids in length; the 3C human rhino virus protease, which comprises about eight amino acids in length; and the PreScission™ cleavage site, which comprises about eight amino acids in length. In an example, an enterokinase recognition site was used to separate an OPH sequence from a fusion partner (Wu, C.-F. et al., 2000b; Wu, C.-F. et al., 2001b).

In an eukaryotic expression system (e.g., a fungal expression system), the “terminator region” or “terminator” may also comprise a specific DNA sequence that permits site-specific cleavage of the new transcript so as to expose a polyadenylation site. This signals a specialized endogenous polymerase to add a stretch of adenosine nucleotides (“polyA”) of about 200 in number to the 3′ end of the transcript. RNA molecules modified with this polyA tail appear to more stable and are translated more efficiently. Thus, in other embodiments involving an eukaryote, in some embodiments a terminator comprises a signal for the cleavage of the RNA, and in some aspects the terminator signal promote polyadenylation of the message. The terminator and/or polyadenylation site elements may serve to enhance message levels and/or to reduce read through from the cassette into other sequences.

A terminator contemplated includes any known terminator of transcription, including but not limited to those described herein. For example, a termination sequence of a gene, such as for example, a bovine growth hormone terminator and/or a viral termination sequence, such as for example a SV40 terminator. In certain embodiments, the termination signal may lack of transcribable and/or translatable sequence, such as due to a sequence truncation. In one example, a trpC terminator from Aspergillus nidulans has been used in the expression of recombinant OPH (Dave, K. I. et al., 1994b).

In expression, particularly eukaryotic expression, a polyadenylation signal may be included to effect proper polyadenylation of the transcript. Any such sequence may be employed. Some embodiments include the SV40 polyadenylation signal and/or the bovine growth hormone polyadenylation signal, convenient and/or known to function well in various target cells. Polyadenylation may increase the stability of the transcript and/or may facilitate cytoplasmic transport.

To propagate a vector in a host cell, it may comprise one or more origins of replication sites (“ori”), which comprises a nucleic acid sequence at which replication initiates. Alternatively an autonomously replicating sequence (“ARS”) may be employed if using a yeast host cell.

Various types of prokaryotic and/or eukaryotic expression vectors are known in the art. Examples of types of expression vectors include a bacterial artificial chromosome (“BAC”), a cosmid, a plasmid [e.g., a pMB1/colE1 derived plasmid such as pBR322, pUC18; a Ti plasmid of Agrobacterium tumefaciens derived vector (Rogers, S. G. et al., 1987)], a virus (e.g., a bacteriophage such as a bacteriophage M13, an animal virus, a plant virus), and/or a yeast artificial chromosome (“YAC”). Some vectors, known herein as “shuttle vectors” may employ control sequences that allow it to be replicated and/or expressed in both prokaryotic and eukaryotic cells [e.g., a wheat dwarf virus (“WDV”) pW1-11 and/or pW1-GUS shuttle vector (Ugaki, M. et al., 1991)]. An expression vector operatively linked to a nucleic acid sequence encoding an enzymatic sequence may be constructed using techniques in the art in light of the present disclosures [In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Cell Biology” (Morgan, K. Ed.) John Wiley & Sons, 2002].

Numerous expression systems exist that comprise at least a part or all of the compositions discussed above. Prokaryote- and/or eukaryote-based systems may be employed to produce nucleic acid sequences, and/or their cognate polypeptides, proteins and peptides. Many such systems are widely available, including those provide by commercial vendors. For example, an insect cell/baculovirus system may produce a high level of protein expression of a heterologous nucleic acid sequence, such as described in U.S. Pat. Nos. 5,871,986, 4,879,236, both incorporated herein by reference, and which may be bought, for example, under the name MAxBAc® 2.0 from INVITROGEN® and BACPACK™ BACULOVIRUS EXPRESSION SYSTEM FROM CLONTECH®. In an additional example of an expression system include STRATAGENE COMPLETE CONTROL™ Inducible Mammalian Expression System, which involves a synthetic ecdysone-inducible receptor, or its pET Expression System, an Escherichia coli expression system. Another example comprises an inducible expression system available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. In a specific example, E3 carboxylesterase enzymatic sequences and phosphoric triester hydrolase functional equivalents have been recombinantly expressed in a BACPACK™ Baculovirus Expression System From CLONTECH® (Newcomb, R. D. et al., 1997; Campbell, P. M. et al., 1998). In certain embodiments, a biomolecule may be expressed in a plant cell (e.g., a corn cell), using techniques such as those described in U.S. Pat. Nos. 6,504,085, 6,136,320, 6,087,558, 6034,298, 5,914,123, and 5,804,694.

2. Prokaryotic Expression Vectors and Use

In some embodiments, a prokaryote such as a bacterium comprises a host cell. In specific aspects, the bacterium host cell comprises a Gram-negative bacterium cell. Various prokaryotic host cells have been used in the art with expression vectors, and a prokaryotic host cell known in the art may be used to express a peptide and/or a polypeptide (e.g., a polypeptide comprising an enzyme sequence).

An expression vector for use in prokaryotic cells generally comprises nucleic acid sequences such as, a promoter, a ribosome binding site (e.g., a Shine-Delgarno sequence), a start codon, a multiple cloning site, a fusion partner, a protease cleavage site, a stop codon, a transcription terminator, an origin of replication, a repressor, and/or any other additional nucleic acid sequence that may be used in such an expression vector in the art [see, for example, Makrides, S.C., 1996; Hannig, G. and Makrides, S.C., 1998; Stevens, R. C., 2000; In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Cell Biology” (Morgan, K. Ed.) John Wiley & Sons, 2002].

A promoter may be positioned about 10 to about 100 nucleotides 5′ to a nucleic acid sequence comprising a ribosome binding site. Examples of promoters that have been used in a prokaryotic cell includes a T5 promoter, a lac promoter, a tac promoter, a trc promoter, an araBAD promoter, a P_{L} promoter, a T7 promoter, a T7-lac operator promoter, and variations thereof. The lactose operator regulates the T5 promoter. A lac promoter (e.g., a lac promoter, a lacUV5 promoter), a tac promoter (e.g., a tact promoter, a tacit promoter), a T7-lac operator promoter or a trc promoter are each suppressed by a lacI repressor, a more effective lacI^{Q} repressor and/or an even stronger lacI^{Q1} repressor (Glascock, C. B. and Weickert, M. J., 1998). Isopropyl-β-D-thiogalactoside (“IPTG”) may be used to induce lac, tac, T7-lac operator and trc promoters. An araBAD promoter may be suppressed by an araC repressor, and may be induced by 1-arabinose. A P_{L} promoter or a T7 promoter are each suppressed by a λclts857 repressor, and induced by a temperature of 42° C. Nalidixic acid may be used to induce a P_{L} promoter.

In an example, recombinant amino acid substitution mutants of OPH have been expressed in Escherichia coli using a lac promoter induced by IPTG (Watkins, L. M. et al., 1997b). In another example, recombinant wild type and a signal sequence truncation mutant of OPH was expressed in Pseudomonas putida under control of a lactac and tac promoters (Walker, A. W. and Keasling, J. D., 2002). In a further example, an OPH-Lpp-OmpA fusion protein has been expressed in Escherichia coli strains JM105 and XL1-Blue using a constitutive lpp-lac promoter and/or a tac promoter induced by IPTG and controlled by a lacP repressor (Richins, R. D. et al., 1997; Kaneva, I. et al., 1998; Mulchandani, A. et al., 1999b). In an additional example, a cellulose-binding domain-OPH fusion protein has also been recombinantly expressed under the control of a T7 promoter (Richins, R. D. et al., 2000). In a further example, recombinant Altermonas sp. JD6.5 OPAA has been expressed under the control of a trc promoter in Escherichia coli (Cheng, T.-C. et al., 1999). In an additional example, a (His)6 tag sequence-thrombin cleavage site-squid-type DFPase has been expressed using a Ptac promoter in Escherichia coli (Hartleib, J. and Ruterjans, H., 2001a).

A ribosome binding site functions in transcription initiation, and may be positioned about 4 to about 14 nucleotides 5′ from the start codon. A start codon signals initiation of transcription. A multiple cloning site comprises restriction sites for incorporation of a nucleic acid sequence encoding a peptide and/or a polypeptide of interest.

A stop codon signals translation termination. The vectors and/or the constructs may comprise at least one termination signal. A “termination signal” or “terminator” comprises DNA sequences involved in specific termination of a RNA transcript by a RNA polymerase. Thus, in certain embodiments a termination signal ends the production of a RNA transcript. A terminator may be used in vivo to achieve a desired message level. A transcription terminator signals the end of transcription and often enhances mRNA stability. Examples of a transcription terminator include a rrnB T1 and/or a rrnB T2 transcription terminator (Unger, T. F., 1997). An origin of replication regulates the number of expression vector copies maintained in a transformed host cell.

A selectable marker usually provides a transformed cell resistance to an antibiotic. Examples of a selectable marker used in a prokaryotic expression vector include a δ-lactamase, which provides resistance to antibiotic such as an ampicillin and/or a carbenicillin; a tet gene product, which provides resistance to a tetracycline, and/or a Km gene product, which provides resistance to a kanamycin. A repressor regulatory gene suppresses transcription from the promoter. Examples of repressor regulatory genes include the lacI, the lad', and/or the lacI^{Q1} repressors (Glascock, C. B. and Weickert, M. J., 1998). Often, the host cell's genome, and/or additional nucleic acid vector co-transfected into the host cell, may comprise one or more of these nucleic acid sequences, such as, for example, a repressor.

An expression vector for a prokaryotic host cell may comprise a nucleic acid sequence that encodes a periplasmic space signal peptide. In some aspects, this nucleic acid sequence may be operatively linked to a nucleic acid sequence comprising an enzymatic peptide and/or polypeptide, wherein the periplasmic space signal peptide directs the expressed fusion protein to be translocated into a prokaryotic host cell's periplasmic space. Fusion proteins secreted in the periplasmic space may be obtained through simplified purification protocols compared to non-secreted fusion proteins. A periplasmic space signal peptide may be operatively linked at or near the N-terminus of an expressed fusion protein. Examples of a periplasmic space signal peptide include the Escherichia coli ompA, ompT, and malel leader peptide sequences and the T7 caspid protein leader peptide sequence (Unger, T. F., 1997).

Mutated and/or recombinantly altered bacterium that release a peptide and/or a polypeptide (e.g., an enzyme sequence) into the environment may be used for purification and/or contact of a proteinaceous molecule with a target chemical ligand. For example, a strain of bacteria, such as, for example, a bacteriocin-release protein mutant strain of Escherichia coli, may be used to promote release of expressed proteins targeted to the periplasm into the extracellular environment (Van der Wal, F. J. et al., 1998). In other aspects, a bacterium may be transfected with an expression vector that produces a gene and/or a gene fragment product that promotes the release of a protenaceous molecule of interest from the periplasm into the extracellular environment. For example, a plasmid encoding the third topological domain of TolA has been described as promoting the release of endogenous and recombinantly expressed proteins from the periplasm (Wan, E. W. and Baneyx, F., 1998).

### H. HOST CELLS

Many host cells from various cell types and organisms are available and known in the art. As used herein, the terms “cell,” “cell line,” and “cell culture” may be used interchangeably. All of these terms also include their progeny, which includes any and all subsequent generations. All progeny may not be identical due to deliberate and/or inadvertent mutations. In the context of expressing a heterologous nucleic acid sequence, “host cell” refers to a prokaryotic and/or an eukaryotic cell, and it includes any transformable organism capable of replicating a vector and/or expressing a heterologous gene and/or gene fragment encoded by a vector. A host cell can, and has been, used as a recipient for vectors. A host cell may be “transfected” or “transformed,” which refers to a process by which exogenous nucleic acid sequence may be transferred or introduced into the host cell. A transformed cell includes the primary subject cell and its progeny. Techniques for transforming a cell include, for example calcium phosphate precipitation, cell sonication, diethylaminoethanol (“DEAE”)-dextran, direct microinjection, DNA-loaded liposomes, electroporation, gene bombardment using high velocity microprojectiles, receptor-mediated transfection, viral-mediated transfection, or a combination thereof [In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002].

Once a suitable expression vector may be transformed into a cell, the cell may be grown in an appropriate environment, and in some cases, used to produce a tissue and/or whole multicellular organism. As used herein, the terms “engineered” and “recombinant” cells and/or host cells are intended to refer to a cell comprising an introduced exogenous nucleic acid sequence. Therefore, engineered cells are distinguishable from naturally occurring cells that do not contain a recombinantly introduced exogenous nucleic acid sequence. Engineered cells are thus cells having a nucleic acid sequence introduced through the hand of man. Recombinant cells include those having an introduced cDNA and/or genomic gene and/or a gene fragment positioned adjacent to a promoter not naturally associated with the particular introduced nucleic acid sequence, a gene, and/or a gene fragment. An enzyme or a proteinaceous molecule produced from the introduced gene and/or gene fragment may be referred to, for example, as a recombinant enzyme or recombinant proteinaceous molecule, respectively. All tissues, offspring, progeny and/or descendants of such a cell, tissue, and/or organism comprising the transformed nucleic acid sequence thereof may be used.

Though an expressed proteinaceous molecule may be purified from cellular material, some embodiments disclosed herein use the properties of a proteinaceous molecule composition comprising, a proteinaceous molecule expressed and retained within a cell, whether naturally and/or through recombinant expression. In certain embodiments, a proteinaceous molecule may be produced using recombinant nucleic acid expression systems in the cell. Cells are known herein based on the type of proteinaceous molecule expressed within the cell, whether endogenous and/or recombinant, so that, for example, a cell expressing an enzyme of interest may be known as an “enzyme cell,” a cell expressing a lipase may be known herein as a “lipase cell,” etc. Additional examples of such nomenclature include a carboxylesterase cell, an OPAA cell, a human phospholipase A₁ cell, a carboxylase cell, a cutinase cell, an aminopeptideases cell, etc., respectively denoting cells that comprise, a carboxylesterase, an OPAA, a human phospholipase A₁, a carboxylase, a cutinase, an aminopeptideases, etc.

In some embodiments, a cell comprises a bacterial cell, a fungal cell (e.g., a yeast cell), an animal cell (e.g., an insect cell), a plant cell, an algae cell, a mildew cell, or a combination thereof. In some aspects, the cell comprises a cell wall. Contemplated proteinaceous molecule comprising cell walls include, but are not limited to, a bacterial cell, a fungal cell, a plant cell, or a combination thereof. In some facets, a microorganism comprises the proteinaceous molecule. Examples of contemplated microorganisms include a bacterium, a fungus, or a combination thereof. Examples of a bacterial host cell that have been used with expression vectors include an Aspergillus niger, a Bacillus (e.g., B. amyloliquefaciens, B. brevis, B. lichenifonnis, B. subtilis), an Escherichia coli, a Kluyveromyces lactis, a Moraxella sp., a Pseudomonas (e.g., fluorescens, putida), Flavobacterium cell, a Plesiomonas cell, an Alteromonas cell, or a combination thereof. Examples of a yeast cell include a Streptomyces lividans cell, a Gliocladium virens cell, a Saccharomyces cell, or a combination thereof.

Host cells may be derived from prokaryotes and/or eukaryotes, which may be used for the desired result comprises replication of the vector and/or expression of part or all of the vector-encoded nucleic acid sequences. Numerous cell lines and cultures are available for use as a host cell, and they may be obtained through the American Type Culture Collection, an organization which serves as an archive for living cultures and genetic materials. An appropriate host may be determined based on the vector backbone and the desired result. A plasmid and/or cosmid, for example, may be introduced into a prokaryote host cell for replication of many vectors. Examples of a bacterial cell used as a host cell for vector replication and/or expression include DH5a, JM109, and KCB, as well as a number of commercially available bacterial hosts such as Novablue™ Escherichia coli cells (NOVAGENE®), SURE® Competent Cells and SOLOPACK™ Gold Cells (STRATAGENE®). However, Escherichia coli cells have been the common cell types used to express both wild type and mutant forms of OPH (Dumas, D. P. et al., 1989a; Dave, K. I. et al., 1993; Lai, K. et al., 1994; Wu, C.-F. et al., 2001a). In an example, the OPH I106A/F132A/H257Y and G60A mutants have been expressed in Escherichia coli BL-21 host cells (Kuo, J. M. and Raushel, F. M., 1994; Li, W.-S. et al., 2001). In a further example, maltose-binding domain-E3 carboxylesterase and phosphoric triester hydrolase functional equivalents have been expressed in Escherichia coli TB1 cells (Claudianos, C. et al., 1999). In another example, the OPH mutants designated W131F, F132Y, L136Y, L140Y, H257L, L271Y, F306A, and F306Y each have been expressed in Novablue™ Escherichia coli cells (Gopal, S. et al., 2000). In an additional example, OPAA from Alteromonas sp JD6.5 has been recombinantly expressed in Escherichia coli cells (Hill, C. M., 2000). In a further example, recombinant Altermonas sp. JD6.5 OPAA has been expressed in Escherichia coli (Cheng, T.-C. et al., 1999). In a further example, the mpd gene has been recombinantly expressed in Escherichia coli, and the encoded enzyme demonstrated methyl parathion degradation activity (Zhongli, C. et al., 2001). In an additional example, a recombinant squid-type DFPase fusion protein has been expressed Escherichia coli BL-21 cells (Hartleib, J. and Ruterjans, H., 2001a). Alternatively, bacterial cells such as Escherichia coli LE392 may be used as host cells for phage viruses. Of course, a bacterium species may be selected to express a proteinaceous molecule due to a particular property. In an example, Moraxella sp. that degrades p-nitrophenol, a toxic cleavage product of parathion and methyl parathion, has been used to recombinantly express an OPH-InaV fusion protein. The resulting recombinant bacterial degrades both toxic OP compounds and their cleavage product (Shimazu, M. et al., 2001b).

Examples of eukaryotic host cells for replication and/or expression of a vector include yeast cells HeLa, NIH3T3, Jurkat, 293, Cos, CHO, Saos, and PC12. In an example, OPH has been expressed in the host yeast cells of Streptomyces lividans (Steiert, J. G. et al., 1989). In another example, OPH has been expressed in host insect cells, including Spodoptera frugiperda sf9 cells (Dumas, D. P. et al., 1989b; Dumas, D. P. et al., 1990). In a further example, OPH has been expressed in the cells of Drosophila melanogaster (Phillips, J. P. et al., 1990). In an additional example, OPH has been expressed in the fungus Gliocladium virens (Dave, K. I. et al., 1994b). In a further example, the gene for human paraoxonase, PON1, has been recombinantly expressed in human embryonic kidney cells (Josse, D. et al., 2001; Josse, D. et al., 1999). In a further example, E3 carboxylesterase and phosphoric triester hydrolase functional equivalents have been expressed in host insect Spodoptera frugiperda sf9 cells (Campbell, P. M. et al., 1998; Newcomb, R. D. et al., 1997). In an additional example, a phosphoric triester hydrolase functional equivalent of a butyrylcholinesterase has been expressed in Chinese hamster ovary (“CHO”) cells (Lockridge, O. et al., 1997). In certain embodiments, an eukaryotic cell that may be selected for expression comprises a plant cell, such as, for example, a corn cell.

### I. PRODUCTION OF EXPRESSED PROTEINACEOUS MOLECULES

Any size flask and/or fermentor may be used to grow a cell, a tissue and/or an organism that may express a recombinant proteinaceous molecule. In certain embodiments, bulk production of a composition, an article, etc. comprising an enzymatic sequence is contemplated.

In an example, a fusion protein comprising, N-terminus to C-terminus, a (His)6 polyhistidine tag, a green fluorescent protein (“GFP”), an enterokinase recognition site, and an OPH lacking the 29 amino acid leader sequence, has been expressed in Escherichia coli. The GFP sequence produced fluorescence that was proportional both the quantity of the fusion protein, and the activity of the OPH sequence. The fusion protein was more soluble than an OPH expressed without the added sequences, and was expressed within the cells (Wu, C.-F. et al., 2000b; Wu, C.-F. et al., 2001a).

The temperature selected may influence the rate and/or quality of recombinant proteinaceous molecule production. In some embodiments, expression of a proteinaceous molecule may be conducted at about 4° C. to about 50° C. Such combinations may include a shift from one temperature (e.g., about 37° C.) to another temperature (e.g., about 30° C.) during the induction of the expression of proteinaceous molecule. For example, both eukaryotic and prokaryotic expression of an OPH may be conducted at temperatures about 30° C., which has increased the production of an enzymatically active OPH by reducing protein misfolding and/or inclusion body formation in some instances (Chen-Goodspeed, M. et al., 2001b; Wang, J. et al., 2001; Omburo, G. A. et al., 1992; Rowland, S. S. et al., 1991). In an additional example, a prokaryotic expression of a recombinant squid-type DFPase fusion protein at about 30° C. also enhanced yield of an active enzyme (Hartleib, J. and Ruterjans, H., 2001a). Fed batch growth conditions at 30° C., in a minimal media, using glycerol as a carbon source, may be suitable for expression of various enzymes.

### J. PRODUCTION OF CELLS AND VIRUSES

A technique in the art may be used in the isolation, growth and storage of a virus, a cell, a microorganism, and a multicellular organism from which a biomolecular composition (e.g., an enzyme, a proteinaceous molecule, an antibiological peptide, etc.) may be derived, including those where endogenously and/or recombinantly produces biomolecule may be desired. Such techniques of cell isolation, characterization, genetic manipulation, preservation, small-scale solid medium and/or liquid medium production growth, growth optimization, large (“industrial,” “commercial”) scale production (e.g., batch culture, fed-batch culture) of a biomolecule (“fermentation”), separation of a biomolecule from a cell and/or visa versa, etc. for various cell types (e.g., a microorganism, a bacterial cell, an Eubacteria cell, a fungi, a protozoa cell, an algae cell, an extremophile cell, an insect cell, a plant cell, a mammalian cell, a recombinantly modified virus and/or a cell) are used in the art [see, for example, in “Manual of Industrial Microbiology and Biotechnology, 2^{nd} Edition (Demain, A. L. and Davies, J. E., Eds.), 1999; “Maintenance of Microorganism and Cultured Cells—A Manual of Laboratory Methods, 2^{nd} Edition” (Kirsop, B. E. and Doyle, A., Eds.), 1991; Walker, G. M. “Yeast Physiology and Biotechnology,” 1998; “Molecular Industrial Mycology Systems and Applications for Filamentous Fungi” (Leong, S. A. and Berka, R. M., Eds.), 1991; “Recombinant Microbes for Industrial and Agricultural Applications” (Murooka, Y. and Imanaka, T., Eds.), 1994; “Handbook of Applied Mycology Fungal Biotechnology Volume 4” (Arora, D. K., Elander, R. P., Mukerji, K. G., Eds.), 1992; “Genetics and Breeding of Industrial Microorganisms” (Ball, C., Ed.), 1984; “Microbiological Methods Seventh Edition” (Collins, C. H., Lyne, P. L., Grange, J. M., Eds.), 1995; “Handbook of Microbiological Media” (Parks, L. C., Ed.), 1993; Waites, M. J. et al., “Microbiology—An Introduction,” 2001; “Rapid Microbiological Methods in the Pharmaceutical Industry,” (Easter, M. C., Ed.), 2003; “Handbook of Microbiological Quality Control Pharmaceuticals and Medical Devices” (Baird, R. M., Hodges, N. A., Denyer, S. P., Eds.), 2000; “Bioreactor System Design” (Asenjo, J. A. and Marchuk, J. C., Eds.), 1995; Endress, R. “Plant Cell Biotechnology,” 1994; Slater, A. et al., “Plant Biotechnology—The genetic manipulation of plants,” 2003; “Molecular Cloning” (Sambrook, J., and Russell, D. W., Eds.), 3rd Edition, 2001; and “Current Protocols in Molecular Biology” (Chanda, V. B. Ed.), 2002.]. In embodiments wherein a cell and/or a virus may be pathogenic (e.g., pathogenic to an organism) may be produced, techniques in the art may be used for handling a pathogen, including identification of a pathogen, production of a pathogen, sterilizing a pathogen, attenuating a pathogen, as well as conducting cell and/or virus preparation to reduce the quantity of a pathogen in non-pathogenic material [see, for example, In “Manual of Commercial Methods in Clinical Microbiology” (Truant, A. L., Ed.), 2002; “Manual of Clinical Microbiology 8^{th} Edition Volume 1” (Murray P. R., Baron, E. J., Jorgensen, J. H., Pfaller, M. A., Yolken, R. H., Eds.), 2003; “Manual of Clinical Microbiology 8^{th} Edition Volume 2” (Murray P. R., Baron, E. J., Jorgensen, J. H., Pfaller, M. A., Yolken, R. H., Eds.), 2003; and “Biological Safety Principles and Practice 3^{rd} Edition” (Fleming, D. O. and Hunt, D. L., Eds.), 2000].

In certain embodiments, a cell that endogenously and/or recombinantly produces a biomolecule (e.g., an enzyme) comprising a thermophilic, a psychrophilic and/or a mesophilic cell may be selected to produce a biomolecular composition for use in an environment that matches and/or overlaps the conditions the biomolecule may function. A biomolecule for use in an embodiment may be so selected. For example, a cell (e.g., a plurality of cells) that produce one or more mesophilic lipolytic enzymes, psychrophilic lipolytic enzymes, and/or thermophilic lipolytic enzymes may be incorporated into a material formulation to confer lipolytic activity over a wide range of temperature conditions for use in temperate environmental conditions. In a further example, a cell that endogenously and/or recombinantly produces a thermophilic lipolytic enzyme may be selected for production of a biomolecular composition comprising the thermophilic lipolytic enzyme. In such a case, the biomolecular composition may then be incorporated into a material formulation to confer a lipolytic property in a thermophilic temperature, such as, for example, a coating for use in a kitchen near a stove heating an oil and/or a fat. Examples of a thermophile contemplated for use are shown at the Tables below.

TABLE 6
Examples of an Archaea Thermophile and Culture Source(s)

| Genus (growth range) | Examples of Culture Collection Strain(s) |
|---|---|
| Acidianus (e.g., about 45° C. to about | DSMZ Nos. 3772, 1651 and/or 3191 |
| 96° C.) | |
| Archaeoglobus (e.g., about 65° C. to | DSMZ Nos. 4304, 4139, 5631 and/or 11195 |
| about 95° C.) | |
| Desulfurococcus (e.g., about 70° C. to | DSMZ Nos. 3822, 2161 and/or 2162 |
| about 95° C.) | |
| Hyperthermus (e.g., about 95° C. to | DSMZ No. 5456 |
| about 107° C.) | |
| Metallosphaera (e.g., about 50° C. to | DSMZ Nos. 10039 and/or 5348 |
| about 80° C.) | |
| Methanobacterium (e.g., about 37° C. | DSMZ Nos. 3387, 863, 7095, 5982, 1535, 2611, 11106, |
| to about 68° C.) | 3108, 2257, 11074, 3266 and/or 2956 |
| Methanococcus (e.g., about 35° C. to | DSMZ Nos. 2067, 1224 and/or 1537 |
| about 91° C.) | |
| Methanohalobium (e.g., about 50° C. | DSMZ Nos. 3721 and/or 5814 |
| to about 55° C.) | |
| Methanosarcina (e.g., about 30° C. to | DSMZ Nos. 2834, 14042, 800, 13486, 2053, 12914, |
| about 55° C.) | 3028, 4659, 1825, 2834, and/or 1232, ATCC 35395 |
| Methanothermus (e.g., about 83° C. to | DSMZ Nos. 2088 and/or 3496 |
| about 88° C.) | |
| Methanosaeta (e.g., about 55° C. to | DSMZ Nos. 2139, 3013, 6752, 17206, 4774 |
| about 60° C.) | |
| Methanothrix (e.g., about 35° C. to | DSMZ Nos. 6194 |
| about 65° C.) | |
| Pyrobaculum (e.g., about 74° C. to | DSMZ Nos. 7523, 13514, 4184, 13380 and/or 4185 |
| about 103° C.) | |
| Pyrococcus (e.g., about 70° C. to | DSMZ Nos. 3638, 12428 and/or 3773 |
| about 103° C.) | |
| Pyrodictium (e.g., about 80° C. to | DSMZ Nos. 6158, 2708 and/or 2709 |
| about 110° C.) | |
| Staphylothermus (e.g., about 65° C. to | DSMZ Nos. 12710 and/or 3639 |
| about 98° C.) | |
| Sulfolobus (e.g., about 55° C. to about | DSMZ Nos. 639, 7519, 6482, 5389, 1616T, 1617, 5354, |
| 87° C.) | 5833 and/or 1616 |
| Thermococcus (e.g., about 50° C. to | DSMZ Nos. 11906, 12767, 12819, 10322, 11836, 2476, |
| about 98° C.) | 10152, 12820, 10395, 11113, 5473, 10394, 10343, 9503, |
| | 12597, 12349, 5262, 12768 and/or 2770 |
| Thermofilum (e.g., about 70° C. to | DSMZ Nos. 2475 |
| about 95° C.) | |
| Thermoproteus (e.g., about 70° C. to | DSMZ Nos. 2338, 2078 and/or 5263 |
| about 97° C.) | |

TABLE 7
Examples of a Gram-negative Thermophile and Culture Source(s)

| Genus (growth range) | Examples of Culture Collection Strain(s) |
|---|---|
| Acetomicrobium (e.g., about 58 to about 73° C.) | ATCC Nos. 43122; DSMZ Nos. 20678 and/or |
| | 20664 |
| Chlorobium tepidum (e.g., about 55° C. to about | ATCC Nos. DSMZ No. 245, 266 and/or 269 |
| 56° C.) | |
| Chloroflexus aurantiacus (e.g., about 20 to about | ATCC Nos. 29365 and/or 29366; DSMZ Nos. 635, |
| 66° C.) | 636, 637 and/or 638 |
| Desulfurella (e.g., about 52 to about 57° C.) | ATCC Nos. 51451; DSMZ Nos. 5264, 10409 and/or |
| | 10410 |
| Dichotomicrobium (e.g., about 35 to about 55° C.) | ATCC Nos. 49408; DSMZ No. 5002 |
| Fervidobacterium (e.g., about 40 to about 80° C.) | ATCC Nos. 35602 and/or 49647 |
| Flexibacter (e.g., about 18 to about 47° C.) | ATCC Nos. 23079, 23086, 23087, 23090 and/or |
| | 23103 |
| Isosphaera (e.g., about 35 to about 55° C.) | ATCC Nos. 43644; DSMZ No. 9630 |
| Methylococcus (e.g., about 30 to about 50° C.) | ATCC Nos. 19069 |
| Microscilla (e.g., about 30 to about 45° C.) | ATCC Nos. 23129, 23134, 23182 and/or 23190 |
| Oscillatoria (e.g., about 56 to about 60° C.) | ATCC Nos. 27906 and/or 27930 |
| Thermodesulfobacterium (e.g., about 65 to about | DSMZ Nos. 2178, 12571, 14290, 1276 and/or 8975 |
| 70° C.) | |
| Thermoleophilum (e.g., about 45 to about 70° C.) | ATCC Nos. 35263 and/or 35268 |
| Thermomicrobium (e.g., about 45 to about 80° C.) | DSMZ No. 5159 |
| Thermonema (e.g., about 60 to about 70° C.) | ATCC Nos. 43542; DSMZ Nos. 5718 and/or 10300 |
| Thermosipho (e.g., about 33 to about 77° C.) | DSMZ No. 5309, 13481, 12029 and/or 6568 |
| Thermotoga (e.g., about 55 to about 90° C.) | ATCC Nos. 43589, 51869, BAA-301, BAA-488 |
| | and/or BAA-489 |
| Thermus (e.g., about 70 to about 75° C.) | ATCC Nos. 25105, 27634, 27978, 31556 and/or |
| | 31674 |
| Thiobacillus aquaesulis (e.g., about 40 to about | ATCC Nos. 23642, 23645, 27977 and/or 43788 |
| 50° C.) | |

TABLE 8
Examples of Gram-positive Thermophiles and Culture Sources

| Genus (growth range) | Examples of Culture Collection Strain(s) |
|---|---|
| Clostridium (e.g., about 10° C. to about 65° C.) | ATCC Nos. 10000, 10092, 10132, 10388 |
| | and/or 49002 |
| Desulfotomaculum (e.g., about 20° C. to about 70° C.) | ATCC Nos. 19858, 23193, 49208, 49756 |
| | and/or 700205 |
| Rubrobacter (e.g., about 46° C. to about 48° C.) | ATCC No. 51242; DSMZ Nos. 5868 and/or |
| | 9941 |
| Saccharococcus (e.g., about 68° C. to about 78° C.) | ATCC No. 43124; DSMZ No. 4749 |
| Sphaerobacter (e.g., about 55° C.) | DSMZ No. 20745 |
| Thermacetogenium (e.g., about 55° C. to about 58° C.) | DSMZ No. 12270 |
| Thermoanaerobacter (e.g., about 35° C. to about 78° C.) | ATCC Nos. 31936, 31960, 33488, 35047 |
| | and/or 49915 |
| Thermoanaerobium (e.g., about 45° C. to about 75° C.) | DSMZ Nos. 7040, 1457, 9766, 9003 and/or |
| | 9769 |

Examples of a psychrophile and a culture source include a Moritalla (e.g., ATCC Nos. 15381 and BAA-105; DSMZ No. 14879), a Leifsonia aurea (e.g., DSMZ No. 15303, CIP No. 107785, MTCC No. 4657), and/or a Methanococcoides burtonii (e.g., DSM No.: 6242). Examples of a halophile and a culture source include a Halobacterium (e.g., DSMZ Nos. 3754 and 3750), a Halococcus (e.g., DSMZ Nos. 14522, 1307, 5350, 8989), a Haloferax (e.g., DSMZ Nos. 4425, 4427, 1411, 3757), a Halogeometricum (e.g., DSMZ No. 11551; JCM No. 10706), a Haloterrigena (e.g., DSMZ Nos. 11552, 5511), a Halorubrum (e.g., DSMZ Nos. 10284, 5036, 1137, 3755, 14210, 8800), and/or a Haloarcula (e.g., ATCC 43049, DSMZ Nos. 12282, 4426, 6131, 3752, 11927, 8905, 3756). Examples of a Gram-positive extreme halophile genera with exemplary NaCl growth ranges include an Aerococcus (1.71 M), a Marinococcus (0.09 to 3.42 M), a Planococcus (0.17 to 2.57 M), a Sporohalobacter (0.5 to 2.0 M), a Staphylococcus (1.71 M), or a combination thereof. Examples of a Gram-positive extreme alkaliphile genera with exemplary pH growth ranges include an Aerococcus (pH 9.6), an Amphibacillus (pH 10), an Enterococcus (pH 9.6), an Exiguobacterium (pH 6.5 to 11.5), or a combination thereof. Examples of a Gram-negative extreme halophile with exemplary NaCl growth ranges include a Halobacteroides (1.44 to 2.4 M), a Halomonas (0.09 to 3.42 M) a Marinobacter (0.08 to 3.5 M), or a combination thereof. Examples of a Gram-negative extreme alkaliphile and/or extreme acidophile genera with exemplary pH growth ranges include an Acetobacter (pH 5.4 to 6.3), an Acidomonas (pH 2.0 to 5.5), an Acidiphilium (pH 2.5 to 5.9), an Arthrospira (pH 11.0), a Beijerinckia (pH 3.0 to 10.0), a Chitinophaga (pH 4.0 to 10.0), a Derxia (pH 5.5 to 9.0), an Ectothiorhodospira (pH 7.6 to 9.5), a Frateuria (pH 3.6), a Gluconobacter (pH 5.5 to 6.0), a Herbaspirillum (pH 5.3 to 8.0), a Leptospirillum (pH 1.5 to 4.0), a Morococcus (pH 5.5 to 9.0), a Rhodopila (pH 4.8 to 5.0), a Rhodobaca bogoriensis (pH range 7.5-10; ATCC No. 700920), a Thermoleophilum (pH 5.8 to 8.0), a Thermomicrobium (pH 7.5 to 8.7), a Thiobacillus (pH 2.0 to 8.0), an Xanthobacter (pH 5.8 to 9.0), or a combination thereof. Examples of an Archaea extreme halophile genera with exemplary NaCl growth ranges include a Haloarcula (1.5 to 4.0 M), a Haobacterium (1.5 to 4.0 M), a Halococcus (1.5 to 4.0 M), a Haloferax (1.5 to 4.0 M), a Methanohalobium (0.01 2.0 M), a Methanohalophilus (0.5 to 2.0 M), a Natronobacterium (1.5 to 4.0 M), a Natronococcus (1.5 to 4.0 M), a Pyrodictium (0.02 to 2.05 M), or a combination thereof. Examples of an Archaea extreme alkaliphile and/or an extreme acidophile genera with exemplary pH growth ranges include an Acidianus (pH 1.0 to 6.0), an Archaeoglobus (pH 4.5 to 7.5), a Desulfurococcus (pH 4.5 to 7.0), a Haloarcula (pH 5.0 to 8.0), a Halobacterium (pH 5.0 to 8.0), a Halococcus (pH 5.0 to 8.0), a Haloferax (pH 5.0 to 8.0), a Metallosphaera (pH 1.0 to 4.5), a Methanococcus (pH 5.0 to 9.0), a Methanohalophilus (pH 7.5 to 9.5), a Natronobacterium (pH 8.5 to 11.0), a Natronococcus (pH 8.5 to 11.0), a Pyrobaculum (pH 5.0 to 7.0), a Pyrococcus (pH 5.0 to 7.0), a Pyrodictium (pH 5.0 to 7.0), a Sulfolobus (pH 1.0 to 6.0), a Thermococcus (pH 4.0 to 8.0), a Thermofilum (pH 4.0 to 6.7), a Thermoproteus (pH 2.5 to 6.0), or a combination thereof.

In other embodiments, cells that endogenously and/or recombinantly produce a petroleum lipolytic enzyme may be selected to produce a biomolecular composition, which may be used in a material formulation, such as, for example, for use in aiding removal of a petroleum lipid from an item and/or a surface. Examples of such a microorganism genera and/or a strain contemplated for use in production of a petroleum lipolytic enzyme (e.g., a cell-based particulate material comprising a petroleum lipolytic enzyme) include an Azoarcus [e.g., DSMZ Nos. 12081, 14744, 6898, 9506 (sp. strain T), 15124], a Blastochloris [e.g., DSMZ Nos. 133, 134, 136, 729, 13255 (ToP1)], a Burkholderia (e.g., DSMZ Nos. 9511, 50341, 13243, 13276, 11319), a Dechloromonas (e.g., ATCC No. 700666; DSMZ No. 13637), a Desulfobacterium [ATCC Nos. 43914, 43938, 49792; DSMZ: 6200 (cetonicum strain Hxd3)], a Desulfobacula (e.g., ATCC No. 43956; DSMZ Nos. 3384, 7467), a Geobacter [e.g., DSMZ Nos. 12179, 13689 (grbiciae TACP-2T), 13690 (grbiciae TACP-5), 7210 (metallireducens GS15), 12255, 12127], a Mycobacterium (e.g., ATCC Nos. 10142, 10143, 11152, 11440, 11564), a Pseudomonas (e.g., ATCC Nos. 10144, 10145, 10205, 10757, 27853), a Rhodococcus (e.g., ATCC Nos. 10146, 11048, 12483, 12974, 14346), a Sphingomonas (e.g., DSMZ Nos. 7418, 10564, 1805, 13885, 6014), a Thauera [e.g., DSMZ Nos. 14742, 12138, 12266, 14743, 12139, 6984 (aromatica K172)], a Vibrio (e.g., ATCC Nos. 11558, 14048, 14126, 14390, 15338), or a combination thereof. Examples of a microorganism strain for a petroleum lipolytic enzyme production, and examples of a target substrate following in brackets, include an Azoarcus sp. strain EB1 (e.g., target substrate includes ethylbenzene), an Azoarcus sp. strain T (e.g., toluene, m-xylene), an Azoarcus tolulyticus Td15 (e.g., toluene, m-xylene), an Azoarcus tolulyticus To14 (e.g., toluene), a Blastochloris sulfoviridis ToP1 (e.g., toluene), a Burkholderia sp. strain RP007 (e.g., naphthalene phenanthrene), a Dechloromonas sp. strain JJ (e.g., benzene, toluene), a Dechloromonas sp. strain RCB (e.g., benzene, toluene), a Desulfobacterium cetonicum (e.g., toluene), a Desulfobacterium cetonicum strain AK-01 (e.g., a C₁₃ to C₁₈ alkane), a Desulfobacterium cetonicum strain Hxd3 (e.g., a C₁₂ to C₂₀ alkane, 1-hexadecene), a Desulfobacterium cetonicum strain mXyS1 (e.g., toluene, m-xylene, m-ethyltoluene, m-cymene), a Desulfobacterium cetonicum strain NaphS2 (e.g., naphthalene), a Desulfobacterium cetonicum strain oXyS1 (e.g., toluene o-xylene, o-ethyltoluene), a Desulfobacterium cetonicum strain Pnd3 (e.g., a C₁₄ to C₁₇ alkane, 1-hexadecene), a Desulfobacterium cetonicum strain PRTOL1 (e.g., toluene), a Desulfobacterium cetonicum strain TD3 (e.g., C₆-C₁₆ alkanes), a Desulfobacula toluolica To12 (e.g., toluene), a Geobacter grbiciae TACP-2T (e.g., toluene), a Geobacter grbiciae TACP-5 (e.g., toluene), a Geobacter 7210 metallireducens GS15 (e.g., toluene), a Mycobacterium sp. strain PYR-1 (e.g., anthracene, benzopyrene, fluoranthene, phenanthrene, pyrene, 1-nitropyrene), a Pseudomonas putida NCIB9816 (e.g., naphthalene), a Pseudomonas putida OUS82 (e.g., naphthalene, phenanthrene, a cyclic hydrocarbon), a Pseudomonas sp. strain C18 (e.g., dibenzothiophene, naphthalene, phenanthrene), a Pseudomonas sp. strain EbN1 (e.g., ethylbenzene, toluene), a Pseudomonas sp. strain HdN1 (e.g., a C₁₄ to C₂₀ alkane), a Pseudomonas sp. strain H×N1 (e.g., a C₆-C₈ alkane), a Pseudomonas sp. strain M3 (e.g., toluene, m-xylene), a Pseudomonas sp. strain mXyN1 (e.g., toluene, m-xylene), a Pseudomonas sp. strain NAP-3 (e.g., naphthalene), a Pseudomonas sp. strain OcN1 (e.g., a C₈-C₁₂ alkane), a Pseudomonas sp. strain PbN1 (e.g., ethylbenzene, propylbenzene), a Pseudomonas sp. strain pCyN1 (e.g., p-Cymene, toluene, p-ethyltoluene), a Pseudomonas sp. strain pCyN2 (e.g., p-Cymene), a Pseudomonas sp. strain T3 (e.g., toluene), a Pseudomonas sp. strain ToN1 (e.g., toluene), a Pseudomonas sp. strain U2 (e.g., naphthalene), a Pseudomonas stutzeri AN10 (e.g., naphthalene, 2-methylnaphthalene), a Rhodococcus sp. strain 124 (e.g., indene, naphthalene, toluene), a Sphingomonas paucimobilis var. EPA505 (e.g., anthracene, fluoroanthene, naphthalene, phenanthrene, pyrene), a Thauera aromatica K172 (e.g., toluene), a Thauera aromatica T1 (e.g., toluene), a Vibrio sp. strain NAP-4 (e.g., naphthalene), or a combination thereof.

### K. CELL-BASED BIOMOLECULAR COMPOSITIONS

After production of a living cell, the cell may be used as a biomolecular composition. Such a biomolecular composition may be known herein as a “crude cell preparation”. A crude cell preparation comprises a desired biomolecule (e.g., an active biomolecule such as a lipase), within and/or otherwise in contact with a cell and/or a cellular debris. In certain aspects, the total content of desired biomolecule may range from about 0.0000001% to about 100% of a crude cell preparation, by volume and/or dry weight, depending upon factors such as expression efficiency of the biomolecule in the cell and the amount of processing and/or purification steps. A higher content of desired biomolecule in the biomolecular composition may be selected in specific embodiments when conferring activity to a material formulation. But, in certain embodiments, the biomolecular composition comprises certain cellular components, particularly a cell wall and/or a cell membrane material, to provide material that may be protective to the biomolecule, enhances the particulate nature of the biomolecular composition, or a combination thereof. Thus, the biomolecular composition may comprise about 0.0000001% to about 100% of cellular component(s), by volume and/or dry weight. However, in certain embodiments, lower ranges of cellular component(s) are used, as the biomolecular composition may therefore comprise a greater percentage of a desired biomolecule.

In embodiments wherein the cellular material may be primarily derived from a microorganism, such as through expression of the biomolecule by a microorganism, the biomolecular composition may be known herein as a “microorganism based particulate material.” The association of a biomolecule with a cell and/or a cellular material may be produced through endogenous expression, expression due to recombinant engineering, or a combination thereof. In some embodiments, a crude cell preparation comprises a biomolecule partly and/or whole encapsulated by a cell membrane and/or a cell wall, whether naturally so and/or through recombinant engineering. Such a biomolecule (e.g., the active biomolecule) encapsulated within and/or as a part of a cell wall and/or a cell membrane may be referred to herein as a “whole cell material” or “whole cell particulate material.”

An embodiment of the cell-based particulate material comprises the material in the form of a “whole cell material,” which refers to particulate material resembling an intact living cell upon microscopic examination, in contrast to cell fragments of varying shape and size. Such a whole cell particulate material may encapsulate an expressed biomolecule (e.g., an enzyme) located in and/or internal to a cell wall and/or a cell membrane. In certain aspects, the encapsulation of a biomolecule by a whole cell particle may provide greater protection relative to a biomolecule located on the external surface of a cell and/or otherwise not comprised within and/or encapsulated by a cell wall, a cell membrane, and/or any addition encapsulating material (e.g., a microencapsulating polymeric material). The biomolecule so encapsulated may be protected from a material formulation's component (e.g., a solvent, a binder, a polymer, a cross-linking agent, a reactive chemical such as a peroxide, an additive, etc.); a material formulation related chemical reaction (e.g., thermosetting reaction); a potentially damaging agent that a material formulation may contact (e.g., a chemical, a solvent, a detergent, etc.); or a combination thereof.

A preparation of a cell may comprise a certain percentage of cell fragments, which comprise pieces of a cell wall, a cell membrane, and/or other cell components (e.g., an expressed biomolecule). The whole cell particulate material comprises about 50% to about 100%, of a whole cell material. The percentage of whole cell material and cell fragments may be determined by any applicable technique in the art such as microscopic examination, centrifugation, etc, as well as any technique described herein for determining the properties of a pigment, an extender, and/or other particulate material either alone and/or comprised in a material formulation. In some aspects, cell fragments may be used as a cell-based particulate material. The cell fragment cell-based particulate material comprises about 50% to about 100%, of cell fragment material.

In some embodiments, a multicellular organism (e.g., a plant) may undergo a processing step wherein one or more cells are physically, chemically, and/or enzymatically separated to produce a material with desired properties (e.g., particulate properties) for a material formulation (e.g., a biomolecular composition). In certain embodiments, cells and/or cell components may be separated using a disrupting step, described herein. As microorganisms are generally unicellular and/or oligocellular in nature, they are used in many embodiments, as the number of processing steps used to prepare a cell-based particulate material from such an organism may be fewer than for a cell from a multicellular organism. For example, a particulate material for a material formulation may be selected for properties such as ease of dispersal, particle size, particle shape, etc. A microorganism may be selected for cell shape, cell size, ease of dispersal, due to poor affinity for other cells relative to a cell embedded in a multicellular organism, or a combination thereof, to produce a cell-based particulate material with desired particulate material properties using fewer processing steps and/or with greater ease than a multicellular organism.

In certain embodiments, a cell-based particulate material may comprise various cellular component(s) (e.g., a cell wall material, a cell membrane material, a nucleic acid, a sugar, a polysaccharide, a peptide, a polypeptide, a protein, a lipid, etc.). Such a cell and/or a virus biomolecule component(s) have been described (see, for example, CRC Handbook of Microbiology. Volume 1, bacteria; Volume 2, fungi, algae, protozoa, and viruses; Volume 3, microbial compositions: amino acids, proteins, and nucleic acids; Volume 4, microbial compositions: carbohydrates, lipids, and minerals; Volume 5, microbial products; Volume 6, growth and metabolism; Volume 7, microbial transformation; Volume 8, toxins and enzymes; Volume 9, pt. A. antibiotics—Volume. 9, pt. B. antimicrobial inhibitors; 1977). In certain embodiments, the cell-based particulate material comprises a cell wall and/or a cell membrane material, to enhance the particulate nature of the cell-based particulate material. However, in many aspects the cell-based particulate material comprises a cell wall material, as the cell wall may be the dominant cellular component for conferring particulate material properties such as shape, size, and/or insolubility, etc.

Depending upon the type of processing used various cell components may be partly and/or fully removed from the organism to produce a cell-based particulate material. In particular, a processing step may comprise contacting a cell with a liquid (e.g., an organic liquid) to dissolve a cell component(s). Removal of the solvent may thereby remove (“extract”) the dissolved cell component(s) from the particulate matter. However, a large biomolecule, particularly a polymer comprised as part of a cell wall, such as a peptidoglycan, a teichoic acid, a lipopolysacharide, or a combination thereof, may be resistant to extraction with a non-aqueous and/or an aqueous solvent, and thus be retained as a component of the particulate matter. In particular embodiments, a large biomolecule of greater than about 1,000 kDa molecular mass, may be retained in the particulate matter. Further, in certain embodiments, greater than about 50% of the dry weight of such particulate matter may comprise a large biomolecule of greater than about 1,000 kDa molecular mass, and/or a cell wall polymer, after processing.

A biomolecule, particularly a cell wall polymer, may be at and/or near the interface of the particulate matter and the external environment. As this interface may be primary area of contact between the particulate matter and a material formulation's component(s), such a large biomolecule may contribute to the properties of the particulate matter produced from a cell used in a material formulation. Examples of such properties include the size range of particulate matter, the shape of the particulate matter, the solubility of the particulate matter, the permeability and/or impermeability of the particulate matter to a chemical, the chemical reactivity of the particulate matter, or a combination thereof. A chemical moiety of the large biomolecule at the interface of the particulate matter and the external environment may chemically react with, for example, a component of a material formulation. In certain embodiments, such a reaction may be used, for example, in the chemical cross-linking of a cell-based particulate material to a binder in a thermosetting material formulation. By participating in such a cross-linking reaction, a cell-based particulate material may be selected for use as a component with such a function (e.g., a binder in a coating, a cross-linking agent in a material formulation).

In addition to the biomolecule(s) described herein that are contemplated as contributing to the particulate nature and/or potential chemical reactivity of a cell-based particulate material, such a composition may comprise another biomolecule (e.g., a colorant, an enzyme, an antibody, a receptor, a transport protein, structural protein, a ligand, a prion, an antimicrobial and/or an antifungal peptide and/or polypeptide) that may confer a property to a material formulation. Such a biomolecule may be, for example, an endogenously produced cell component, and/or a product of expression of a recombinant nucleic acid in a virus and/or a cell [see, for example, “Molecular Cloning,” 2001; and “Current Protocols in Molecular Biology,” 2002].

### L. PROCESSING OF CELLS AND EXPRESSED BIOMOLECULES

After production of a biomolecule by a living cell, the composition comprising the biomolecule may undergo one or more processing steps to prepare a biomolecular composition. Examples of such steps include concentrating, drying, applying physical force, extracting, resuspending, controlling temperature, permeabilizing, disrupting, chemically modifying, encapsulating, proteinaceous molecule purification, immobilizing, or a combination thereof. Various embodiments of a biomolecular composition are contemplated after one or more such processing steps. However, each processing step may increase economic costs and/or reduce total desired biomolecule yield, so that embodiments comprising fewer steps may reduce costs. The order of steps may be varied and still produce a biomolecular composition.

A biomolecule prepared as a crude cell preparation (e.g., a whole cell particulate material) may have greater stability and/or other property (e.g., chemical resistance, temperature resistance, etc.) than a preparation wherein the biomolecule has been substantially separated from a cell membrane and/or a cell wall. A biomolecule prepared as a crude cell preparation, wherein the biomolecule may be localized between a cell wall and a cell membrane and/or within the cell so that the cell wall and/or a cell membrane separates the biomolecule from the extracellular environment, may have greater stability than a preparation wherein the biomolecule has been substantially separated from a cell membrane and/or a cell wall.

1. Sterilization/Attenuation

A processing step may comprise sterilizing a biomolecular composition. Sterilizing (“inactivating”) kills living matter (e.g., a cell, a virus), while attenuation reduces the virulence of a living matter. A sterilizing and/or attenuating step may be used as continued post expression growth of a cell, a virus, and/or a contaminating organism may detrimentally affect the composition. For example, in some embodiments, one or more properties of a material formulation may be undesirably altered by the presence of a living organism. Additionally, sterilizing reduces the ability of a living recombinant organism to be introduced into the environment, in an embodiment wherein such an event is undesirable. A biomolecular composition may be designated by the type of processing step and nature of the composition, such as, for example, a cell-based particulate material wherein the majority of material by dry weight, wet weight and/or volume has been sterilized or attenuated, may be known herein as a “sterilized cell-based particulate material” or “attenuated cell-based particulate material,” respectively. In another example, a purified enzyme that has been sterilized may be referred to as a “sterilized purified enzyme,” and so forth.

In certain embodiments, it contemplated that sterilization and/or attenuation may be accomplished in or on a material formulation (e.g., a coating, a biomolecular composition) by contact with biologically detrimental component of such items such as a solvent and/or chemically reactive component (e.g., a thermosetting binder, a cross-linking agent). In further embodiments, sterilizing and/or attenuation of a material formulation (e.g., a cell-based particulate material) comprising such a material may be accomplished by any method known in the art, and are commonly applied in the food, medical, and pharmaceutical arts to sterilize and/or attenuate pathogenic microorganisms [see, for example, “Food Irradiation: Principles and Applications,” 2001; “Manual of Commercial Methods in Clinical Microbiology” (Truant, A. L., Ed.), 2002; “Manual of Clinical Microbiology 8^{th} Edition Volume 1” (Murray P. R., Baron, E. J., Jorgensen, J. H., Pfaller, M. A., Yolken, R. H., Eds.), 2003; “Manual of Clinical Microbiology 8^{th} Edition Volume 2” (Murray P. R., Baron, E. J., Jorgensen, J. H., Pfaller, M. A., Yolken, R. H., Eds.), 2003; and “Biological Safety Principles and Practice 3^{rd} Edition” (Fleming, D. O. and Hunt, D. L., Eds.), 2000]. Examples of sterilizing and/or attenuating may include contacting the living matter with a toxin, irradiating the living matter, heating the living matter above a temperature suitable for life (e.g., 100° C. in many cases, more for an extremophile), or a combination thereof. In some embodiments sterilizing and/or attenuating comprises irradiating the living matter, as radiation generally does not leave a toxic residue, and may not detrimentally affect the stability of a desired biomolecule (e.g., a colorant, an enzyme) that might be present in the cell-based particulate material, to the same degree as other sterilizing and/or attenuating techniques (e.g., heating). Examples of radiation include infrared (“IR”) radiation, ionizing radiation, microwave radiation, ultra-violet (“UV”) radiation, particle radiation, or a combination thereof. Particle radiation, UV radiation and/or ionizing radiation may be used in some embodiments, and particle radiation may be used in some facets. Examples of particle radiation include alpha radiation, electron beam/beta radiation, neutron radiation, proton radiation, or a combination thereof.

The pathogenicity of a cell and/or a virus may be reduced and/or eliminated through genetic alteration (e.g., an attenuated virus with reduced pathogenicity, infectivity, etc.), processing techniques such as partial or complete sterilization and/or attenuation using techniques in the art (e.g., heat treatment, irradiation, contact with chemicals), passage of a virus through cell not typically a host cell for the virus, or a combination thereof, and such a cell and/or a virus may be used in some facets. In many embodiments, the majority (e.g., about 50% to about 100%) of the cell-based particulate material has been sterilized and/or attenuated, with 100% or as close to 100% as may be practically accomplishable, selected for specific facets.

However, in alternative embodiments, a partly sterilized, partly attenuated, a non-sterilized and/or attenuated biomolular composition (e.g., a cell-based particulate material) may be suitable for a temporary material formulation (e.g., a surface treatment with a relatively reduced service life, a temporary coating). In particular aspects, the damage produced by a living cell and/or a virus in a material formulation may make the material formulation more suitable for use as a temporary material formulation. For example, inclusion and/or contact with a cell-based particulate material may reduce the durability (e.g., degrade a binder molecule, degrade a surface treatment's component) of a material formulation (e.g., a coating, a coating produced film) over time, enhancing ease of removal, degradation, damage, and/or destruction (e.g., reducing resistance to a liquid component, abrasion, etc.) of a material formulation to produce an item (e.g., a manufactured article, a composition), for example, with a relatively reduced service life.

2. Concentrating

A processing step may comprise concentrating a biomolecular composition. As used herein, “concentrating” refers to any process reducing the volume of a composition, an article, etc. Often, an undesired component that comprises the excess volume is removed; the desired composition may be localized to a reduced volume, or a combination thereof.

For example, a concentrating step may be used to reduce the amount of a growth and/or expression medium component from a biomolecular composition. Nutrients, salts and other chemicals that comprise a biological growth and/or expression medium may be unnecessary and/or unsuitable in a material formulation, and reducing the amount of such compounds may be done. A growth medium may promote microorganism growth in a material formulation, while salt(s) and/or other chemical(s) may alter the formulation of a material formulation.

Concentrating a biomolecular composition (e.g., cell-based particulate material) may be by any method known in the art, including, for example, washing, filtrating, a gravitational force, a gravimetric force, or a combination thereof. An example of a gravitational force comprises normal gravity. An example of a gravimetric force comprises the force exerted during centrifugation. Often a gravitational and/or a gravimetric force may be used to concentrate a biomolecular composition from undesired components that are retained in the volume of a liquid medium. After desired biomolecule(s) (e.g., cell based particulate materials) are localized to the bottom of a centrifugation devise, the media may be removed via such techniques as decanting, aspiration, etc.

3. Drying

In additional embodiments, the biomolecular composition may be dried. Such a drying step may remove an undesired liquid, such as from a cell-based particulate material. Examples of drying include freeze-drying, lyophilizing, spray drying, or a combination thereof. In some aspects, a cryoprotectant may be added to the biomolecular composition during a drying step (e.g., lyophilizing). In certain embodiments, a drying step may enhance the particulate nature of the material. For example, reduction of a liquid in the cell-based particulate material may reduce the tendency of particles of the material to adhere to each other (e.g., agglomerate, aggregate), or a combination thereof. In some aspects, the particulate material comprise a form (e.g., a powder) sufficiently liquid free (“dry”) that it may be suitable for convenient storage at ambient and/or other temperature conditions without desiccation.

4. Physical Force

An application of physical force (e.g., grinding, milling, shearing) may enhance the particulate nature of the material by converting a multicellular material (e.g., a plant) into an oligocellular and/or a unicellular material; and/or convert an oligocellular material into a unicellular material. Such an application of physical force may be referred to as “milling” herein, such as, for example, in the claims. Further, the average particle size may be reduced to a desired range, including the conversion of cell(s) into disrupted cell(s) and/or cell debris. Such a physical force may produce a powder form, such as a power of a cell-based particulate material. Physical force may also be used in processing steps dealing with a purified and/or a semi-purified biomolecule (e.g., an enzyme, such as a powdered enzyme).

5. Extraction

A biomolecule may be removed by extraction of a biomolecular composition (e.g., a cell-based particulate material). For example, a lipid and/or an aqueous component of a cell-based particulate material may be partly or fully removed by extraction with appropriate solvents. Such extraction may be used to dry the cell-based particulate material by removal of liquid (e.g., water, lipids), remove of a biotoxin, sterilize/attenuate living material in the composition, disrupt and/or permeablize a cell, alter the physical and/or chemical characteristics of the cell-external environment interface, or a combination thereof. For example, a lipid such as a phospholipid are often present at and/or within a cell wall, a cell membrane, and/or an other cellular membrane (e.g., an organelle membrane), and an extraction step may partly or fully remove a lipid that may chemically react with a component of a material formulation. Additionally, such an extraction of a surface lipid may alter (e.g., increase, decrease) the hydrophobicity and/or hydrophilicity of, for example, a cell-based particulate material to enhance its suitability (e.g., disperability) for a material formulation.

6. Resuspending

A purification step may comprise resuspending a precipitated composition comprising a biomolecule (e.g., a desired enzyme) from a cell debris. For example, in certain embodiments, a composition comprising a coating and an enzyme prepared by the following steps: obtaining a culture of cells that express the enzyme; concentrating the cells and removing the culture media; disrupting the cell structure; drying the cells; and adding the cells to the coating. In some aspects, the composition may be prepared by the additional step of suspending the disrupted cells in a solvent prior to adding the cells to the coating.

In certain aspects, the composition may be prepared by adding the cell culture powder to glycerol, admixing with glycerol and/or suspending in glycerol. In other facets, the glycerol may be at a concentration of about 50%. In specific facets, the cell culture powder comprised in glycerol at a concentration of about 3 mg of the milled powder to about 3 ml of about 50% glycerol. In certain facets, the composition may be prepared by adding the powder comprised in glycerol to the paint at a concentration of about 3 ml glycerol comprising powder to 100 ml of paint. The powder may also be added to a liquid component such as glycerol prior to addition to the paint. The numbers are exemplary only and do not limit the use. The concentration was chosen merely to be compatible with the amount of substance that may be added to one example of paint without affecting the integrity of the paint itself. Any compatible amount may used.

A processing step may comprise resuspending the composition comprising a biomolecular composition (e.g., a cell-based particulate material). The material to be resuspended may have undergone a prior processing step, such as concentration (e.g., precipitation), drying, extraction, etc., and may be resuspended into a form suitable for storage, further processing, and/or addition to a material formulation. In certain aspects, the resuspension medium may be a liquid component of a material formulation described herein, a cryopreservative (“cryoprotector”), a xeroprotectant, a biomolecule stabilizer, or a combination thereof. A cryopreservative reduces the ability of a cell wall and/or a cell membrane to rupture, particularly during a freezing and thawing process, and typically comprises a liquid; while a xeroprotectant reduces damage to a composition (e.g., a biomolecular composition), during a drying process (e.g., a drying processing step, physical film formation of a coating), and typically comprises a liquid. A biomolecule stabilizer comprises a composition (e.g., a chemical) added to enhance a property such as stability of a biomolecule (e.g., an enzyme). In some embodiments, a cryopreservative, a xeroprotectant, a biomolecule stabilizer, or a combination thereof, may be used as an additive to a material formulation (e.g., a biomolecular composition). Examples of a cryopreservative include glycerol, dimethyl sulfoxide (“DMSO”), a protein (e.g., an animal serum albumin), a sugar of 4 to 10 carbons (e.g., sucrose), or a combination thereof. Examples of a xeroprotectant include glycerol, a glycol such as a polyethylene glycol (e.g., PEG₈₀₀₀), a mineral oil, a bicarbonate (e.g., ammonium bicarbonate), DMSO, a sugar of about 4 to about 10 carbons (e.g., trehalose), or a combination thereof. Often, a cryopreservative, a biomolecule stabilizer, and/or a xeroprotectant comprise an aqueous liquid, and may comprise a pH buffer (e.g., a phosphate buffer). A substance (e.g., a cryopreservative, a xeroprotectant, a biomolecule stabilizer) included as part of a material formulation (e.g., a biomolecular composition) may alter a physical (e.g., hydrophobicity, hydrophilicity, dispersal of particulate material, etc.) and/or a chemical property (e.g., reactivity with a material formulation's component) of a material formulation, and the formulation of such an item may be improved using the techniques described herein and/or the art to account for such a substance on and/or comprised within/as a component of a material formulation. In certain embodiments, the amount of cryopreservative, a biomolecule stabilizer, and/or a xeroprotectant may comprise 0.000001% to 99.9999%, of a biomolecular composition. In specific facets, a biomolecular composition, a cryopreservative, a biomolecule stabilizer, and/or a xeroprotectant may comprise 0.000001% to 66% a glycerol and/or a glycol (e.g., a polyethylene glycol). In other embodiments, a biomolecular composition, a cryopreservative, a biomolecule stabilizer, and/or a xeroprotectant may comprise 0.000001% to 10% DMSO. In further embodiments, a material formulation (e.g., a biomolecular composition) and/or a component thereof such as a cryopreservative, a biomolecule stabilizer, and/or a xeroprotectant may comprise 0.000001 M to 1.5 M bicarbonate.

7. Temperatures

In some embodiments, a processing step may comprise maintaining a biomolecular composition (e.g., a composition comprising an enzyme) at a temperature at or less than the optimum temperature for the activity of a living organism and/or a biomolecule (e.g., a proteinaceous biomolecule) that may detrimentally affect a proteinaceous molecule. For example, often about 37° C. may be the maximum temperature for the processing of a human biomolecule (e.g., an enzyme). Thus temperatures at or less than about 37° C. are contemplated in such aspects, during processing of materials derived from a human cell. Controlling the range of temperatures a biomolecular composition may be exposed to and/or reached by the biomolecular composition during processing may be modified accordingly for a thermophile, a mesophile, and/or a psychrophile derived biomolecular composition.

8. Permeabilization/Disruption

In some aspects, a biomolecular composition comprises a cell preparation (e.g., crude cell, whole cell, etc.) wherein the cell membrane and/or the cell wall has been altered through a permeabilizing process, a disruption process, or a combination thereof. An example of such an altered cell preparation includes a crude cell, a disrupted cell, a whole cell, permeabilized cell, or a combination thereof. As used herein, a “disrupted cell” comprises a cell preparation wherein the cell membrane and/or the cell wall has been altered through a disruption process. As used herein, a “permeabilized cell” comprises a cell preparation wherein the cell membrane and/or the cell wall has been altered through a permeabilizing process. Permeabilization and/or disruption may promote the separation of cells, reduce the average particle size of the material, allow greater access to a biomolecule in a cell (e.g., to promote ease of extraction), or a combination thereof.

A processing step may comprise a permeabilizing step, such as contacting a cell with a permeabilizing agent such as DMSO, ethylenediaminetetraacetic acid (“EDTA”), tributyl phosphate, or a combination thereof. A permeabilizing step may increase the mass transport of a substance (e.g., a ligand) into the interior of a cell where, for example a binding interaction with a biomolecule may occur, such as an enzyme localized inside the cell catalyzes a chemical reaction with the substance. (Martinez, M. B. et al., 1996; Martinez, M. B. et al., 2001; Hung, S.-C. and Liao, J. C., 1996), or a ligand binding a protenaceous molecule (e.g., a peptide, a polypeptide). Cell permeabilizing using EDTA has been described (Leduc, M. et al., 1985).

In some embodiments, a processing step comprises disrupting a cell. A cell may be disrupted by any method known in the art, including, for example, a chemical method, a mechanical method, a biological method, or a combination thereof. Examples of a chemical cell disruption method include suspension in a liquid component (e.g., a solvent) for certain cellular components. In specific facets, such a solvent may comprise an organic solvent (e.g., acetone), a volatile solvent, or a combination thereof. In a particular facet, a cell may be disrupted by acetone (Wild, J. R. et al., 1986; Albizo, J. M. and White, W. E., 1986). In certain facets, the cells are disrupted in a volatile solvent for ease in evaporation. Examples of a mechanical cell disruption method include pressure (e.g., processing through a French press), sonication, mechanical shearing, or a combination thereof. An example of a pressure cell disruption method includes processing through a French press. Examples of a biological cell disruption method include contacting the cell with one or more proteins and/or polypeptides that are known to possess such disrupting activity including a porin and/or an enzyme such as a lysozyme, as well as contact/cell infection with a virus that weakens, damages, and/or permeabilizes a cell membrane, a cell wall, or a combination thereof. In another example, a cell-based particulate material comprising cell(s) and/or cellular component(s) may be homogenized, sheared, undergo one or more freeze thaw cycles, be subjected to enzymatic and/chemical digestion of a cellular material (e.g., a cell wall, a sugar, etc.), undergo extraction with a liquid component (e.g., an organic solvent, an aqueous solvent), etc., to weaken interactions between the cellular material(s). A processing step may comprise sonicating a composition. Other disrupting and/or drying may be done by freeze-drying with a reduced and/or absent cryoprotector (e.g., a sugar).

9. Chemical Modification

In certain embodiments, a biomolecular composition (e.g., a cell based particulate material) may be chemically modified for a physical (e.g., hydrophobicity, hydrophilicity, dispersal of particulate material, etc.) and/or a chemical property (e.g., reactivity with a material formulation's component) to enhance suitability in a material formulation. In embodiments wherein a cell based particulate material may be used, such a chemical modification (e.g., organic chemistry) may primarily affect a cell-external environment interface. Such modifications include for example, acylatylation; amination; hydroxylation; phosphorylation; methylation; adding a detectable label such as a fluorescein isothiocyanate; covalent attachment of a poly ethylene glycol; a derivation of an amino acid by a sugar moiety, a lipid, a phosphate, a farnysyl group; or a combination thereof, as well as others in the art [see, Greene, T. W. and Wuts, P. G. M. “Productive Groups in Organic Synthesis,” Second Edition, pp. 309-315, John Wiley & Sons, Inc., USA, 1991; and co-pending U.S. patent application Ser. No. 10/655,345 “Biological Active Coating Components, Coatings, and Coated surfaces, filed Sep. 4, 2003; in “Molecular Cloning,” 2001; “Current Protocols in Molecular Biology,” 2002]. Additional modifications, particularly those more suited for a purified biomolecule (e.g., a proteinaceous molecule) are described herein.

10. Encapsulation

Additionally, a biomolecular composition (e.g., a cell based material, an antimicrobial peptide, an antifungal peptide, an enzyme, a proteinaceous material) may be encapsulated (e.g., microencapsulated, such as for use in a material formulation), using a microencapsulation technique. Such encapsulation may enhance and/or confer the particulate nature of the biomolecular composition; provide protection to the biomolecular composition; stabilize a biomolecular composition; increase the average particle size to a desired range; allow slow and/or controlled release from the encapsulating material of a component such as a cellular component (e.g., a biomolecule such as an enzyme, an antimicrobial peptide, etc.) and/or an additional encapsulated material (e.g., a chemical preservative/pesticide, an isolated biomolecule, etc.); alter surface charge, hydrophobicity, hydrophilicity, solubility and/or disperability of a biomolecular composition (e.g., a particulate material) and/or an additional encapsulated material; or a combination thereof. For example, an encapsulating material (e.g., an encapsulating membrane) may provide protection to the peptide from peptidase(s), protease(s), and/or other peptide bond and/or side chain modifying substance. In another example, a polyester microsphere may be used to encapsulate and stabilize a biomolecular composition (e.g., a peptide) in a paint composition during storage, or to provide for prolonged, gradual release of the biomolecular composition after it is dispersed in a paint film covering a surface. In another example, an antibiological agent's activity (e.g., antifungal activity) may be controlled and/or stabilized by microencapsulating an antibiological proteinaceous molecule (e.g., a peptide) to enhance their stability in a material formulation such as, for example, a liquid coating composition and in the final paint film or coat, and may to provide for a prolonged, gradual release of the proteinaceous molecule after it is dispersed in a paint film covering a surface that may be vulnerable to attachment and growth of a cell (e.g., a fungal cell, a spore).

Examples of microencapsulation (e.g., microsphere) compositions and techniques are described in, for example, Wang, H. T. et al., 1991; and U.S. Pat. Nos. 4,324,683, 4,839,046, 4,988,623, 5,026,650, 5153,131, 6,485,983, 5,627,021 and 6,020,312. Other microencapsulation methods which may be employed are those described in U.S. Pat. Nos. 5,827,531; 6,103,271; and 6,387,399. Examples of a microencapsulating material includes a gelatin, a hydrogenated vegetable oil, a maltodextrin, a polyurea, a sucrose, an acacia, an amino resin, an ethylcellulose, a polyester, or a combination thereof. In some facets, an encapsulating material (e.g., a polymer) swells, dissolves, and/or degrades upon contact with a liquid component, a chemical, a biomolecule (e.g., an enzyme), the environment, or a combination thereof. For example, a polyvinyl alcohol, which comprises a water soluble polymer, may be used to encapsulate a peptide antifungal agent for incorporation into a bathroom caulk to allow greater release of the peptide/ease of contact with a microorganism, upon contact of the caulk with moisture/water during the normal use of the caulk.

11. Other Processing Steps/Biomolecule Purification

In other embodiments, a biomolecule (e.g., a proteinaceous molecule) may comprise a purified biomolecule. For example, a “purified proteinaceous molecule” as used herein refers to any proteinaceous molecule removed in any degree from other extraneous materials (e.g., cellular material, nutrient or culture medium used in growth and/or expression, etc). In certain aspects, removal of other extraneous material may produce a purified biomolecule (e.g., a purified enzyme) wherein its concentration has been enhanced about 2 to about 1,000,000-fold or more, from its original concentration in a material (e.g., a recombinant cell, a nutrient or culture medium, etc). In other embodiments, a purified biomolecule may comprise about 0.0000001% to about 100% of a composition comprising a biomolecule. The degree or fold of purification may be determined using any method known in the art or described herein. For example, techniques such as measuring specific activity of a fraction by an assay described herein, relative to the specific activity of the source material, and/or fraction at an earlier step in purification, may be used.

Some techniques for preparation of a biomolecule (e.g., a purified proteinaceous molecule) are described herein. However, one or more additional methods for purification of biologically produced molecule(s) (e.g., ammonium sulfate precipitation, ultrafiltration, polyethylene glycol suspension, hexanol extraction, methanol precipitation, Triton X-100 extraction, acrinol treatment, isoelectric focusing, alcohol treatment, acid treatment, acetone precipitation, etc.) that are known in the art and/or described herein may be used to obtain a purified proteinaceous molecule [Azzoni, A. R. et al., 2002; In “Current Protocols in Molecular Biology” (Chanda, V. B. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Cell Biology” (Morgan, K. Ed.) John Wiley & Sons, 2002; In “Current Protocols in Pharmacology” (Taylor, G. Ed.) John Wiley & Sons, 2002; In “Current Protocols in Cytometry” (Robinson, J. P. Ed.) John Wiley & Sons, 2002; In “Current Protocols in Immunology” (Coico, R. Ed.) John Wiley & Sons, 2002; In “Methods and Molecular Biology, Volume 109 Lipase and Phospholipase Protocols.” (Mark Doolittle and Karen Reue, Eds.), 1999; pancreatic lipase via recombinant expression in a baculoviral system in “Methods and Molecular Biology, Volume 109 Lipase and Phospholipase Protocols.” (Mark Doolittle and Karen Reue, Eds.), 1999; In “Lipases their Structure, Biochemistry and Application” (Paul Woolley and Steffen B. Peterson, Eds.), 1994; Brockerhoff, Hans and Jensen, Robert G. “Lipolytic Enzymes,” 1974; “Lipases” (Borgstrom, B. and Brockman, H. L., Eds), 1984; In “Lipases and Phospholipases in Drug Development from Biochemistry to Molecular Pharmacology.” (Müller, G. and Petry, S. Eds.), 2004]. For example, a biological material comprising a proteinaceous molecule may be homogenized, sheared, undergo one or more freeze thaw cycles, be subjected to enzymatic and/chemical digestion of cellular materials (e.g., cell walls, sugars, etc), undergo extraction with organic and/or aqueous solvents, etc, to weaken interactions between the proteinaceous molecule and other cellular materials and/or partly purify the proteinaceous molecule. In another example, a processing step may comprise sonicating a composition comprising an enzyme.

Cellular materials may be further fractionated to separate a proteinaceous molecule from other cellular components using chromatographic e.g., affinity chromatography (e.g., antibody affinity chromatography, lectin affinity chromatography), fast protein liquid chromatography, high performance liquid chromatography “HPLC”), ion-exchange chromatography, exclusion chromatography; and/or electrophoretic (e.g., polyacrylamide gel electrophoresis, isoelectric focusing) methods. A proteinaceous molecule may be precipitated using antibodies, salts, heat denaturation, centrifugation and the like. A purification step may comprise dialyzing a composition comprising a biomolecule from cell debris. For example, heparin-Sepharose chromatography has been used to enhance purification of lipolytic enzymes such as diacyglycerol lipase, triacylglycerol lipase, lipoprotein lipase, phospholipase A₂, phospholipase C, and phospholipase D [see for example, in “Methods and Molecular Biology, Volume 109 Lipase and Phospholipase Protocols” (Mark Doolittle and Karen Reue, Eds.), pp. 133-143, 1999]. Such processing and/or purification steps are often applicable to various other biomolecules that may be purified. Of course, the techniques used in purifying and identifying a given biomolecule may be applied as appropriate. Additionally, various commercial vendors typically provide purified biomolecule (e.g., an enzyme), often comprising about 90% to about 100% of a specific biomolecule.

For example, the molecular weight of a proteinaceous molecule may be calculated when the sequence is known, and/or estimated when the approximate sequence and/or length is known. SDS-PAGE and staining (e.g., Coomassie Blue) has been commonly used to determine the success of recombinant expression and/or purification of OPH, as described (Kolakowski, J. E. et al., 1997; Lai, K. et al., 1994).

12. Immobilization

Immobilization refers to attachment (i.e., by covalent and/or non-covalent interactions) of a proteinaceous molecule (e.g., an enzyme) to a solid support (“carrier”) and/or cross-linking an enzyme (e.g., a CLEC). For example, immobilization of an enzyme generally refers to covalent attachment of the enzyme to a material's surface at the molecular level or scale, to limit conformational changes in the presence of a solvent that result in loss of activity, prevent enzyme aggregation, improve enzyme resistance to proteolytic digestion by limiting conformational change(s) and/or exposure of cleavage site(s), to increase the surface area of an exposed enzyme to a substrate for catalytic activity, or a combination thereof [In “Engineering of/with Lipases” (F. Xavier Malcata., Ed.) pp. 457-458, 1996; “Methods in non-aqueous enzymology” (Gupta, M. N., Ed.) p. 37, 2000]. In another example, immobilization of an enzyme may be used to improve stability against oxidation (e.g., autooxidation); reduce denaturation upon contact with a solvent, a solute, and/or a shear force; reduce self digestion; prevent loss of an enzyme by dissolving, suspension, etc into a liquid component (e.g., water, a solvent) and being washed away; and providing an increased concentration of an enzyme in a local area for highest yield of a product of enzyme activity. Often other properties such ligand (e.g., substrate) selectivity and/or binding property(s); pH and temperature optimums; kinetic properties such as Km; etc. may be altered by immobilization. Various types of substrates for biomolecule immobilization include a reverse micelle, a zeolite, a Celite Hyflo Supercel, an anion exchange resin, a Celite® (diatomaceous earth), a polyurethane foam particle, a macroporous polypropylene Accurel® EP 100, a macroporous packing particulate, a macroporous anionic resin bead, a polypropylene membrane, an acrylic membrane, a nylon membrane, a cellulose ester membrane, a polyvinylidene difuoride membrane, a filter paper, a teflon membrane, a ceramic membrane, a polyamide, a cellulose hollow fibre, a resin, a polypropylene membrane pretreated with a blocked copolymer, an immunoglobins via enzyme-linked immunosorbent assay, an agarose, an ion-exchange resin, and/or a sol-gel (In “Engineering of/with Lipases” (F. Xavier Malcata., Ed.) pp. 298, 408, 409, 414, 422, 447, 448, 451, 461, 494, 501, 516, 546, 549, 1996; U.S. Pat. No. 4,939,090; Lopez, M. et al., 1998; “Methods in non-aqueous enzymology” (Gupta, M. N., Ed.) pp. 41-51, 63-65, 2000]. For example, a lipase incorporated in sol-gel had 100-fold improved activity (Reetz, M. et al., 1995). For example, though many immobilized lipolytic enzymes comprise a purified enzyme, an immobilized whole cell lipase biocatalyst have been described [In “Engineering of/with Lipases” (F. Xavier Malcata., Ed.), p. 88, 1996]. In another example, in some cases, an enzyme and/or a cell may be immobilized by entrapment into a gel formed from an alginate, a carragenan, and/or a polyacrylamide (Karube, I. et al., 1985; Qureshi, N. et al., 1985; Umemura, I. et al., 1984; Fukui, S, and Tanaka, A. 1984; Mori, T. et al., 1972; Martinek, K. et al., 1977).

A method of immobilization includes, for example, absorption, ionic binding, covalent attachment, cross-linking, entrapment into a gel, entrapment into a membrane compartment, or a combination thereof (Kurt Faber, “Biotransformations in Organic Chemistry, a Textbook, Third Edition.” pp. 345-356, 1997). A lysine amino moiety, an aspartate carboxyl moiety and/or a glutamate carboxyl moiety may be used to chemically bind a proteinaceous molecule to a solid support. For example, a nitrobenzenic acid derivate may be used to acylate the active side lysine of a phospholipase A₂ to improve activity, and immobilize the enzyme to a Reacti-Gel [see for example, in “Methods and Molecular Biology, Volume 109 Lipase and Phospholipase Protocols” (Mark Doolittle and Karen Reue, Eds.), pp. 303-307, 1999]. Immobilization of an epoxy-activated Candida rugosa lipase produces monoalkylation of a lysine moiety(s) that improves enzyme stability by enhancing resistance to other chemical reactions, and modifies substrate selectivity (Kurt Faber, “Biotransformations in Organic Chemistry, a Textbook, Third Edition” Springer-verlag Berlin Heidelberg, p. 313, 1997; Beger, B. and Faber, 1991).

Absorption may be used, for example, to attach a proteinaceous molecule onto a material where it may be held by a non-covalent (e.g., hydrogen bonding, Van der Weals forces) interaction. Examples of a material that may be used for absorption of a proteinaceous molecule (e.g., an enzyme) include a woodchip, an activated charcoal, an aluminum oxide, a diatomaceous earth (e.g., Celite), a cellulose material, a controlled pore glass, a siliconized glass bead, or a combination thereof. For example, in some cases, the buffering capacity of an immobilization carrier, such as a diatomaceous earth (e.g., Celite), may improve the catalytic rate or selectivity of a lipolytic enzyme (e.g., a Pseudomonas sp. lipase), as an acid produced by ester hydrolysis may alter local pH to detrimentally effect the reaction (Kurt Faber, “Biotransformations in Organic Chemistry, a Textbook, Third Edition.”, p. 114-115, 1997; “Lipases” (Borgstrom, B. and Brockman, H. L., Eds), p. 196, 1984].

An ion exchange resin, such as a cation (e.g., carboxymethyl cellulose, Amberlite IRA) resin, an anion (e.g., sephadex, diethyl-aminoethylcellulose) resin, or a combination thereof, may be used to immobilize a biomolecule (e.g., a proteinaceous molecule, an enzyme). Covalent bonding immobilization generally involves chemical reactions on an amino acid residue at an amino moiety (e.g., lysine's epsilon amino group), a phenolic moiety, a suflhydryl moiety, a hydroxyl moiety, a carboxy moiety, or a combination thereof, usually with a spacer chemical that may be used to bind to the proteinaceous molecule to a carrier. Examples of a carrier that may be used to immobilize a proteinaceous molecule by a covalent bond include porous glass via a spacer (e.g., an aminoalkylethoxy-chlorosilane, an aminoalkyl-chlorosilane); a polysaccharide polymer carrier (e.g., agarose, chitin, cellulose, dextran, starch) via reaction cyanogens bromide reactions; a synthetic co-polymer (e.g., polyvinyl acetate) via an epichlorohydrin activation reactions; an epoxy-activate resin; a cation exchange resin activated to covalently bond by acid chloride conversion of a carboxylic acid, or a combination thereof.

A cross-linking enzyme may comprise an enzyme interconnect to a like and/or a different enzyme, via a bifunctional agent (e.g., a glutardialdehyde, dimethyl adipimidate, dimethyl suberimidate and hexamethylenediisocyanate), sometimes with larger molecule such as a proteinaceous molecule (e.g., a “filler protein”) (e.g., an albumin) separating the enzyme(s) molecule(s). This technique may be adapted to other biomolecules(s) (e.g., a proteinaceous molecule, a peptide, a polypeptide, an antibody, an receptor, etc.), and may be used to modify the size of a component. In certain embodiments, an enzyme may be in the form of a crystal. In other aspects, one or more enzyme crystals may be cross-linked to from a CLEC (Hoskin, F. C. G. et al., 1999; Lalonde, J. J. et al., 1995; Persichetti, R. A., 1996). Gel entrapment includes incorporation of a biomolecule (e.g., an enzyme) and/or a cell into a gel matrix (e.g., an alginate, a carragenan gel, a polyacrylamide gel, or a combination thereof) that may be formed into various shapes (Karube, I. et al., 1985; Qureshi, N. et al., 1985; Umemura, I. et al., 1984; Fukui, S, and Tanaka, A. 1984; Mori, T. et al., 1972; Martinek, K. et al., 1977; Kurt Faber, “Biotransformations in Organic Chemistry, a Textbook, Third Edition.” pp. 350-352, 1997). Membrane entrapment refers to restricting the space a biomolecule (e.g., an enzyme) functions in by being placed in a compartment, often imitating the separation of a biomolecule (e.g., an enzyme) that occurs inside a living cell (e.g., localization of an enzyme inside an organelle). An examples of membrane entrapment composition include a micelle, a reversed micelle, a vesicle (e.g., a liposome), a synthetic membrane (e.g., a polyamide, a polyethersulfone) with a pore size smaller than the sequestered biomolecule (e.g., a membrane enclosed enzymatic catalysis or “MEEC”). However, a MEEC may reduce the function of many lipolytic enzymes, possibly due to interference with the interfacial activation process by this type of environment (Kurt Faber, “Biotransformations in Organic Chemistry, a Textbook, Third Edition.” pp. 345-356, 1997).

In some embodiments, a proteinaceous molecule (e.g., a peptide) and/or a property (e.g., antifungal activity) of the proteinaceous molecule may be stabilized in a material formulation (e.g., a paint, a coating) by immobilization (e.g., attachment, linking, tethering, and/or conjugation) to another molecule. For example, a proteinaceous molecule (e.g., a peptide, an enzyme) may be conjugated to a soluble and/or an insoluble carrier molecule to modify the proteinaceous molecule's and/or the carriers solubility properties (e.g., aqueous solubility) as desired. Examples of a carrier molecule that are typically soluble include certain polymer(s) (e.g., a polyethyleneglycol, a polyvinylpyrrolidone). Alternatively, a proteinaceous molecule) may be chemically linked, tethered, and/or conjugated to an insoluble molecule. Examples of a carrier typically insoluble include sand, a silicate, and/or certain polymer(s) (e.g., a polystyrene, a cellulosic polymer, a polyvinylchloride). In some embodiments, the molecular size of the conjugated polymer chosen for conjugating with a proteinaceous molecule (e.g., an antifungal peptide) may be suited for carrying out the desired function in the material formulation (e.g., a coating). Techniques and materials for conjugating a proteinaceous molecule (e.g., a peptide) to other molecules described herein and/or of the art (e.g., the literature), may be used.

In some embodiments, a biomolecular composition (e.g., a proteinaceous molecule, an antibiotic proteinaceous composition, an antibiotic peptide) may comprise an immobilization carrier (e.g., a microsphere, a liposome, a soluble carrier, an insoluble carrier) and/or a carrier material to promote handling, dispersion in a material formulation and/or localization to a part of a material formulation (e.g., a saline solution, a buffer, a solvent). In certain aspects, a immobilization carrier and/or a carrier material may be one suitable for a permanent, a semi-permanent, and/or a temporary material formulation (e.g., a permanent surface coating application, a semi-permanent coating, a non-film forming coating, a temporary coating). In many embodiments, an immobilization carrier and/or a carrier material may be selected to comprise a chemical and/or a physical characteristic which does not significantly interfere with the selected property (e.g., antibiotic activity) of a biomolecular composition (e.g., a proteinaceous molecule, a peptide). For example, a microsphere carrier may be effectively utilized with a proteinaceous composition in order to deliver the composition to a selected site of activity (e.g., onto a surface). In another example, a liposome may be similarly utilized to deliver an antibiotic (e.g., a labile antibiotic). In a further example, a saline solution, a material formulation (e.g., a coating) acceptable buffer, a solvent, and/or the like may also be utilized as a carrier material for a proteinaceous (e.g., a peptide) composition.

### M. INCORPORATION OF A BIOMOLECULAR COMPOSITION INTO A MATERIAL FORMULATION

A component (e.g., a biomolecular composition, a ligand for a biomolecule, an additive) may be incorporated (e.g., embedded) within a material formulation (e.g., a polymeric matrix) via several methods. These methods include, for example, direct addition to a material formulation, incorporation as a component of a de novo formulation during preparation, post preparation absorption, in situ incorporation, post polymerization incorporation, or a combination thereof, and may be used a substitute for, or in combination with, the other techniques described herein for processing (e.g., encapsulation) and incorporation of a component (e.g., an enzyme such as a lipase such as a Candida Antarctica Lipase B “CALB,” a proteinaceous molecule, an antimicrobial peptide) into a material formulation (e.g., a coating, a base paint, a primer coating, an overcoat). The incorporation method selected may influence biomolecule's activity (e.g., binding activity, enzymatic activity). The various assays described herein and/or in the art in light of the present disclosure, may be used to determine the biomolecule's activity (e.g., a fungal resistance property) as part of a composition (e.g., a coating, a film, etc.).

In some embodiments, a material formulation may comprise a component such as a biomolecular composition (e.g., an enzyme, a proteinaceous molecule), a substrate for an enzyme, a ligand (e.g., a binding component), an additive that may affect the activity and/or function of a biomolecular composition (e.g., an enzyme inhibitor, a cofactor, a buffer, etc.), and/or another additive (e.g., a colorant), etc., wherein the component may be incorporated as part of a material formulation during preparation, production, post-cure, manufacture, and/or at a later point in time, such as during service life use. A biomolecular composition (e.g., an antifungal peptidic agent) may function as an additional component to a material formulation [e.g., a previous material formulation such as a commercially available product comprising certain component(s) and/or range(s) of component content], and/or may substitute for all and or part of one or more component(s) of a material formulation (e.g., an antifungal peptidic agent substitution of some or all of a non-peptidic or chemical antifungal component). In certain aspects, a material formulation may be free and/or comprise a reduced content of component(s) (e.g., a chemical, an additive) that are toxic a non-target organism (e.g., a humans, certain animals, certain plants, etc.) and/or that fail to comply with applicable environmental safety rule and/or guideline. In some aspects, a biomolecular composition may work in combination with and/or synergistically with a component (e.g., a synthetic component, a naturally produced component) of a material formulation (e.g., an antibiological enzyme and/or an antibiological peptide combined with a preservative).

A material formulation may undergo a chemical reaction and/or comprise a component that may partly or fully damage, inhibit, and/or inactivate an active biomolecule (e.g., an enzyme). For example, a surface treatment such as a coating (e.g., a polyurethane) may cure by a chemical reaction. In some embodiments, the biomolecular composition (e.g., an enzyme, a peptide, a cell-based particulate material) may be incorporated after the bulk of a chemical reaction in a material formulation has occurred. The bulk of these reactions typically occur during typically material preparation, are known as “body time,” “curing,” “cure time,” etc, with some residual reactions occurring after cure that may be not considered significant to the potential detrimental influence on a biomolecular composition. Incorporation of the material after part or the majority of this main cure time may serve to protect the biomolecular composition from these reactions. These cure times are typically know (e.g., described in manufacturers instruction) and/or readily determined by standard assays for a material and/or an enzyme properties. In some embodiments, the biomolecular composition may be incorporated after about 0%, to about 100% of the cure time has passed. For example, an enzyme such as a lysozyme may be incorporated by admixing after about 80% or more of a body time as passed for a polyurethane coating. In another example, a biomolecular composition may be incorporated post-cure (e.g., after about 90% curing has occurred) for a thermoset. In another embodiment, a biomolecular composition may be incorporated during post-cure processing. In other embodiments, a biomolecular composition may be incorporated after about 100% of the cure time has passed.

Additionally, a biomolecular composition may comprise a plurality of biomolecules and/or a protective material to protect the desired biomolecule(s) from damage by a chemical reactions and/or a component of a material formulation. For example, an enzyme such as a lysozyme may comprise an additional egg white protein that protects the enzyme from loss of activity by a chemical reaction. In another example, a partly purified enzyme, cell-fragment particulate material, whole cell particulate material, an encapsulated biomolecular composition (e.g., an encapsulated purified enzyme, an encapsulated cell-fragment particulate material, etc), an immobilized enzyme, and the like, are used as they provide additional biomolecules and/or a protective material (e.g., an encapsulation material) that may protect the desired biomolecule from a chemical reaction and/or a component of a material formulation, protect the desired biomolecule from damage during normal use (e.g., environmental damage, washings, etc) of a material formulation, or a combination thereof.

In some embodiments, a proteinaceous molecule (e.g., an antifungal peptide) may be chemically linked and/or bonded (e.g., covalently linked, ionically associated) to a component (e.g., a polymer) of a material formulation (e.g., a plastic, a coating, a coating produced film) to incorporate a biomolecular composition into a material formulation. For example, that ability to link a proteinaceous molecule to a polymeric carrier may also be used for chemically linking or otherwise associating one or more antibiological proteinaceous molecules (e.g., an antifungal peptide) to a polymeric material (e.g., a plastic fabric) which would otherwise be more susceptible to infestation, defacement and/or deterioration by a cell (e.g., a fungus). Conventional techniques for linking the N- or C-terminus of a peptide to a long-chain polymer may be employed. For example, an antibiological proteinaceous molecule (e.g., an antifungal peptide) may include additional amino acids on the linking end to facilitate linkage to the polymer (e.g., a PVC polymer). PVC is only one of many types of a polymeric material (e.g., a plastic) that may be linked to a proteinaceous molecule (e.g., an antifungal peptide) in this manner. In a specific example, a PVC-membrane such as a flexible and/or retractable roof and/or covering for an outdoor stadium, may be treated to chemically link an antifungal peptide to at least a portion of the outer surface of the membrane prior to its installation. Where an installed polymer membrane covering may be already infested by mold, and it may be not practical for it to be removed and replaced by an antifungal peptide-linked polymer membrane, it may be feasible to clean the existing infestation and/or discoloration, and then apply and/or bond a suitable antifungal surface treatment (e.g., a coating) comprising a stabilized antifungal peptide.

In other facets, incorporation of a component may be conducted using electric charge, such as by contact of a material formulation with a liquid comprising an electrically charged component, and using electrophoresis to promote movement of the additional component on and/or into the material formulation.

1. Multipacks/Kits

For a purpose such as ease of production, a material formulation (e.g., an antifungal paint, a coating product comprising an antifungal peptidic agent) may be provided to a consumer as a single premixed formulation. In some embodiments, the components of a material formulation may be stored separately prior to combining for use. For example, a fungal-prone surface treatment may be stored in a separate container prior to application, in order to minimize the occurrence of fungal contamination prior to use and for other reasons. In another example, separation of conventional coating components may be typically done to reduce film formation during storage for certain types of coatings.

For a purpose such as to optimize the initial activity (e.g., the activity of a biomolecular composition component) and/or extend the useful lifetime of the material formulation (e.g., an antifungal coating), a biomolecular composition (e.g., an antifungal peptidic agent) may instead be packaged separately from the material formulation (e.g., a paint, a coating product) into which the biomolecular composition (e.g., an antifungal agent) may be added/incorporated. Thus, in certain embodiments, one or more components (e.g., a biomolecular composition), of a material formulation may be stored separately (e.g., a kit of components) prior to combining.

The components may be stored in two or more containers (“pot”) (e.g., about 2 to about 20 containers) in a multipack kit. In certain embodiments, a material formulation (e.g., a coating comprising a biomolecular composition) comprises a multi-pack material formulation, such as a two-pack material formulation (“two-pack kit”), a three-pack material formulation, four-pack material formulation, five-pack material formulation, or more wherein the material formulation components are stored in separate containers. In some embodiments, a multipack material formulation comprises one or more additional container(s) storing the biomolecular composition and/or another component, relative to another material formulation that does not comprise a biomolecular composition. For example, an additional component suitable for use with the biomolecular component (e.g., a solid carrier and/or a liquid carrier suitable for increased stability of a peptidic agent) may be included as part of the material formulation, the separately packaged biomolecular composition, and/or may be separately packaged for addition/incorporation. Separate storage may reduce, for example, microoganism growth in a component (e.g., a coating component), damage to the biomolecular composition by a component (e.g., a coating component), increase the storage life (“pot life”) of material formulation (e.g., a coating), reduce the amount of a preservative in a material formulation (e.g., a coating), allow separate and/or sequential incorporation of a component into a material formulation (e.g., addition of a component post-cure, addition of a component during service life), or a combination thereof. In certain aspects, about 0.000001% to about 100%, including all intermediate ranges and combinations thereof, of one component of a material formulation (e.g., a biomolecular composition, an antifungal composition) may be stored in a separate container from another component of a material formulation. For example, a material formulation may be in the form of a precursor material (e.g., a thermosetting coating that cures into a film) in a container, and a container comprising a biomolecular composition to be combined (e.g., admixed, etc.) with the precursor material for use (e.g., application of a surface treatment to a surface). For example, a new antifungal composition may be prepared at or near the time of use by combining a fungal-prone material (e.g., carbon polymer-containing binder) with other coating components, including an antifungal peptide, polypeptide or protein, as described herein.

In another example, a coating may be stored in a container (“pot”) prior to application. In certain aspects, the coating comprises a multi-pack coating wherein different components of the coating are stored in a plurality of containers (e.g., a kit). Typically, this reduces film formation during storage for certain types of coatings. The components are admixed prior to and/or during application. In certain facets, the coating component(s) of a container holding the biomolecular composition material may further include a coating component such as a preservative, a wetting agent, a dispersing agent, a liquid component, a rheological modifier, or a combination thereof. A preservative may reduce growth of a microoganism, whether the microoganism is derived from the biomolecular composition and/or a contaminating microorganism. It is contemplated that a wetting agent, a dispersing agent, a liquid component, a rheological modifier, or a combination thereof, may promote ease of admixing of coating components in a multi-pack coating. In certain aspects, a three-pack coating or four-pack coating may be used, wherein the first container and the second container comprises coating components separated to reduced film formation during storage, and a third container comprises about 0.001% to about 100%, including all intermediate ranges and combinations thereof, of the biomolecular composition. In certain facets, a multi-pack coating may be used to separate two or more preparations of the biomolecular composition.

2. Assays for Biomolecular Activity in a Material Formulation

In general embodiments, a material formulation comprising a biomolecular composition comprising a desired biomolecule (e.g., a colorant, an enzyme, a peptide), whether endogenously or recombinantly produced, that may alter and/or confer a desired property to the material formulation (e.g., a surface treatment, a filler). As used herein, “activity,” “active,” and/or “bioactivity” refers to a desired property such as color, enzymatic activity, binding activity, antimicrobial activity, antifouling activity, etc, conferred to a material formulation by a biomolecular composition. As used herein, “bioactivity resistance” refers to the ability of a biomolecular composition to confer a desired property during and/or after contact with a stress condition normally assayed for in a standard assay procedure for a material formulation. Examples of such a stress condition includes, for example, a temperature (e.g., a baking condition), contact with a material formulation component (e.g., an organic liquid component), contact with a chemical reaction (e.g., thermosetting film formation), contact with damaging agent to a material formulation (e.g., weathering, detergents, and/or solvents for a paint film), etc. In specific facets, wherein a biomolecular composition comprises a desired biomolecule, a biomolecule may possess a greater bioactivity resistance such as determined with such an assay procedure.

Such bioactivity resistance may be determined using a standard procedure for material formulation described herein or in the art, in light of the present disclosures. In an example, any assay described herein or in the art in light of the present disclosures may be used to determine the bioactivity resistance wherein an enzyme retains detectable enzymatic activity upon contact with a condition typically encountered in a standard assay. Additionally, in certain aspects, it is contemplated that a material formulation comprising an enzyme may lose part of all of a detectable, desirable bioactivity during the period of time of contact with standard assay condition, but regain part or all of the enzymatic bioactivity after return to non-assay conditions. An example of this process is the thermal denaturation of an enzyme at an elevated temperature range into a configuration with lowered or absent bioactivity, followed by refolding of an enzyme, upon return to a more suitable temperature range for the enzyme, into a configuration possessing part or all of the enzymatic bioactivity detectable prior to contact with the elevated temperature. In another example, an enzyme may demonstrate such an increase in bioactivity upon removal of a solvent, a chemical, etc.

In some embodiments, an enzyme identified as having a desirable enzymatic property for one or more target substrates may be selected for incorporation into a material formulation. The determination of an enzymatic property may be conducted using any technique described herein or in the art, in light of the present disclosures. For example, the determination of the rate of cleavage of a substrate, with or without a competitive or non-competitive enzyme inhibitor, can be utilized in determining the enzymatic properties of an enzyme, such as Vₘₐₓ, Kₘ, K_{cat}/Kₘ and the like, using analytical techniques such as Lineweaver-Burke analysis, Bronsted plots, etc Brockerhoff, Hans and Jensen, Robert G. “Lipolytic Enzymes”, pp 10-24, 1974; Dumas, D. P. et al., 1989a; Dumas, D. P. et al., 1989b; Dumas, D. P. et al., 1990; Caldwell, S. R. and Raushel, F. M., 1991c; Donarski, W. J. et al., 1989; Raveh, L. et al., 1992; Shim, H. et al., 1998; Watkins, L. M. et al., 1997a; diSioudi, B. et al., 1999; Hill, C. M., 2000; Hartleib, J. and Ruterjans, H., 2001b; Lineweaver, H. and Burke, D., 1934; Segel, I. H., 1975). Such analysis may be used to identify an enzyme with a specifically enzymatic property for one or more substrates, given that use of an assay for an enzyme's activity may be incorporated with identification of a proteinaceous molecule as having enzymatic activity.

For example, lipolytic enzymes and phosphoric triester hydrolases have demonstrated the ability to degrade a wide variety of lipids and OP compounds, respectively. Methods for measuring the ability of an enzyme to degrade a lipid or an OP compound are described herein as well as in the art. Any such technique may be utilized to determine enzymatic activity of a composition for a particular lipid or an OP compound. For example, techniques for measuring the enzymatic degradation for various lipids comprising an ester and/or other hydrolysable moiety, including a triglyceride such as a triolein, an olive oil, and/or a tributyrin; a chromogenic substrate such as 4-methylumbelliferone, and/or a 4-methylumbelliferone; and/or a radioactively labeled glycerol ester substrate, such as a glycerol [³H]oleic acid esters; may be used (see, for example, Brockerhoff, Hans and Jensen, Robert G. “Lipolytic Enzymes.” pp-25-34, 1974). To measure a lipolytic enzyme's activity against a substrate, a molecular monolayer of a lipid substrate may be used to control variables such as pressure, charge potential, density, interfacial characteristics, enzyme binding, and/or the effects of an inhibitor, in measuring lipolytic enzyme kinetics [see for example, Gargouri, Y. et al., 1989; Melo, E. P. et al., 1995; In “Methods and Molecular Biology, Volume 109 Lipase and Phospholipase Protocols.” (Mark Doolittle and Karen Reue, Eds.), pp 279-302, 1999].

In an additional example, measuring the activity, stability, and other property(s) of a lipolytic enzyme may be conducted using techniques in the art. For example, methods for measuring the activity of a phospholipase A₂ and a phospholipase C by the thin layer chromatography product separation, the fluorescence change of a labeled substrate (e.g., a dansyl-labeled glycerol, a pyrene-PI, a pyrene-PG), the release of product(s) from a radiolabled substrate (e.g., [³H]Plasmenylcholine) have been described [see for example, in “Methods and Molecular Biology, Volume 109 Lipase and Phospholipase Protocols.” (Mark Doolittle and Karen Reue, Eds.), pp. 1-17, 31-48, 1999]. Similarly, the release of fluorogenic product(s) from substrate(s) such as, for example, a 1-trinitrophenyl-aminododecanoyl-2-pyrenedecanoyl-3-O-hexadecyl-sn-glycerol, or a radioactive product(s) from radiolabled substrate(s) such as, for example, a [³H]triolein; glycerol tri[9,10(n)-[³H]oleate; cholesterol-[1⁻¹⁴C]-oleate; a 1(3)-mono-[³H]oleoyl-2-O-mono-oleyleglycerol (a.k.a. [³H]-MOME) and a 1(3)-mono-oleoyl-2-O-mono-oleylglycerol (a.k.a. MOME); by lipolytic enzyme(s) that catalyze hydrolysis of a tri, a di, or a monoacylglycerol(s) and/or sterol ester(s) may be used to measure such enzymes' activity [see for example, in “Methods and Molecular Biology, Volume 109 Lipase and Phospholipase Protocols.” (Mark Doolittle and Karen Reue, Eds.), pp. 18-30, 59-121, 1999]. Other assays using radiolabeled E. coli membranes to measure phospholipase activity in comparison to photometric and other assays has also been described [In “Esterases, Lipases, and Phospholipases from Structure to Clinical Significance.” (Mackness, M. I. and Clerc, M., Eds.), pp 263-272, 1994].

In some cases, these techniques may be modified by replacement of a purified and/or an immobilized enzyme typically assayed with a material formulation, to assay and characterize the enzymatic activity of such a material formulation. Such measurements of the enzymatic activity of compositions may be used to select a material formulation with the desired activity properties of stability, activity, and such like, in different environmental conditions (e.g., pressure, interfacial characteristics, the effects of an inhibitor, temperature, detergent, organic solvent, etc.) and/or after contact with different substrate(s) (e.g., contact with substrates mimicking vegetable oil properties vs. those for a sterol when assaying for a lipolytic enzyme) to assess properties such as the substrate preference, enantiomeric specificity, kinetic properties, etc. of a material formulation.

Techniques for measuring the kinetics of enzymatic degradation for various OP-compounds comprising a P—S bond at the phosphorous center (e.g., an OP-phosphonothiolate) such as a VX [“EA 1701,” “TX60,” “O-ethyl-S-(diisopropylaminoethyl)methylphosphonothioate”], a Russian VX [“R-VX,” “O-isobutyl-S-(diisopropylaminoethyl)methylphosphonothioate”], a tetriso [“O,O-diisopropyl S-(2-diisoprpylaminoethyl) phosphorothiolate”], an echothiophate (“phospholine,” “O,O-diethyl-phosphorothiocholine”), a malathion [“phosphothion,” “S-(1,2-dicarbethoxyethyl)-O,O-dimethyl dithiophosphate”], a dimethoate [“Cygon®,” “Dimetate®,” “O,O-dimethyl-S—(N-methylcarbomoyl-methyl)phosphorodithioatel, an EA 5533 [“OSDMP,” “O,S-diethyl methylphosphonothioate”], an IBP (“Kitazin P,” “O,O-diisopropyl-5-benzylphosphothioate”), an acephate (“O,S-dimethyl acetyl phosphoroamidothioate”), an azinophos-ethyl [“S-(3,4-dihydro-4-oxobenzo[d)-1,2,3-triazin-3-ylmethyl-O,O-diethyl) phosphorothioate”], a demeton S [“VX analogue,” “O,O -diethyl-S-2-ethylthio]ethyl phosphorothioate”], a malathion [“Phosphothion,” “S-(1,2-dicarbethoxyethyl)-O,O-dimethyl dithiophosphate”] and/or a phosalone [“O,O-diethyl-S-(6-chloro-2-oxobenzoxazolin-3-yl-methyl) phosphorodithioate”], of the art may be used (see, for example, diSioudi, B. D. et al., 1999; Hoskin, F. C. G. et al., 1995; Watkins, L. M. et al., 1997a; Kolakowski, J. E. et al., 1997; Gopal, S. et al., 2000; and Rastogi, V. K. et al., 1997).

Techniques for measuring the kinetics of enzymatic detoxification for various OP-compounds comprising a P—F bond at the phosphorous center (e.g., an OP-phosphonofluoridate) such as a soman (“1,2,2-trimethylpropyl-methylphosphonofluoridate”), a sarin (“isopropylmethylphosphonofluoridate”), a DFP (“O,O-diisopropyl phosphorofluoridate”), an alpha (“1-ethylpropylmethylphosphonofluoridate”), and/or a mipafox (“N,N′-diisopropylphosphorofluorodiamidate”) have been described (see, for example Dumas, D. P. et al., 1990; Li, W.-S. et al., 2001; diSioudi, B. D. et al., 1999; Hoskin, F. C. G. et al., 1995; Gopal, S. et al., 2000; and DeFrank, J. and Cheng, T., 1991).

A technique for measuring the kinetics of enzymatic detoxification for an OP-compound comprising a P—CN bond at the phosphorous center (e.g., an OP-phosphonocyanate) such as a tabun (“ethyl N,N-demethylamidophosphorocyanidate”) has been described (see, for example, Raveh, L. et al., 1992).

Techniques for measuring the kinetics of enzymatic detoxification for various OP-compounds comprising a P—O bond at the phosphorous center (e.g., an OP-triester) such as a paraoxon (“diethyl p-nitrophenylphosphate”), the soman analogue O-pinacolyl p-nitrophenyl methylphosphonate, the sarin analogue O-isopropyl p-nitrophenyl methylphosphonate, a NPPMP (“p-nitrophenyl-o-pinacolyl methylphosphonate”), a coumaphos [“O,O-diethyl O-(3-chloro-4-methyl-2-oxo-2H-1-benzyran-7-yl)phosphorothioate], a cyanophos [“O,O-dimethyl p-cyanophenyl phosphorothioatel, a diazinon (“O,O -diethyl O-2-iso-propyl-4-methyl-6-pyrimidyl phosphorothiate”), a dursban (“O,O-diethyl O-3,5,6-trichloro-2-pyridyl phosphorothioate”), a fensulfothion {“O,O-diethyl [p-(methylsulfinyl)phenyl]phosphorothioate”}, a parathion (“O,O-diethyl O-p-nitrophenyl phosphorothioate”), a methyl parathion (“O,O-dimethyl p-nitrophenyl phosphorothioate”), an ethyl parathion [“O,O-diethyl-O-(4-nitrophenyl)phosphorothioate”], an EPN (“O-ethyl 0-(4-nitrophenyl)phenylphosphonothioate”), a DEPP (“diethylphenylphosphate”), NPEPP (“p-nitrophenylethylphenylphosphinate”) have been described (see, for example, Dumas, D. P. et al., 1990; Li, W.-S. et al., 2001; diSioudi, B. D. et al., 1999; Watkins, L. M. et al., 1997a; Gopal, S. et al., 2000; Mulbry, W. and Karns, J., 1989; Hong, S.-B. and Raushel, F. M., 1996; and Dumas, D. P. et al., 1989b).

In one example, the cleavage rate of a phosphonothiolate OP substrate comprising a P—S bond can be measured using a method known as the Ellman reaction. Such substrates may produce a P—S bond cleavage product comprising a free thiol group, which can chemically react with the Ellman's reagent, 5,5′-dithio-bis-2-nitrobenzoic acid (“DTNB”). This reaction produces a 5′-thiol-2-nitrobenzoate anion with a maximum absorbency at 412 nm. P—S cleavage can be determined by the appearance of the free thiol group, measured using a spectrophotometer (Rastogi, V. H. et al., 1997; Gopal, S. et al., 2000; diSioudi, B. et al., 1999; Watkins, L. M. et al., 1997a; Hoskin, F. C. G. et al., 1995; Chae, M. Y. et al., 1994; Ellman, G. L. et al., 1961).

In an additional example, the cleavage of an OP substrate can be measured by detecting the production of a cleavage product comprising a released ion. In a further example, the cleavage of a phosphonofluoridate can be measured by the release of cleavage product comprising a fluoride ion (F⁻) using a fluoride ion specific electrode and a pH/mV meter (Hartleib, J. and Ruterjans, H., 2001a; Gopal, S. et al., 2000; diSioudi, B. et al., 1999; Watkins, L. M. et al., 1997a; DeFrank, J. and Cheng, T., 1991; Dumas, D. P. et al., 1990; Dumas, D. P. et al., 1989a). In another example, the cleavage of a phosphonocyanate can be measured by the release of a cleavage product comprising a cyanide ion (CN⁻) using a cyanide selective electrode with a pH meter (Raveh, L. et al., 1992).

In another example, cleavage of an OP substrate can be measured, for example, by ³¹P NMR spectroscopy. For example, the disappearance of a VX and the formation of the cleavage product ethyl methylphosphonic acid (“EMPA”), has been measured using this technique (Kolakowski, J. E. et al., 1997; Lai, K. et al., 1995). In another example, the disappearance of a tabun and the appearance of the N,N-dimethylamindophosphosphoric acid cleavage product has been measured by ³¹P NMR spectroscopy (Raveh, L. et al., 1992). In a further example, the disappearance of a DFP and appearance of a F cleavage product has been determined using ¹⁹F⁻ and ³¹P NMR spectroscopy (Dumas, D. P. et al., 1989a).

The cleavage of many OP compounds' such as a paraoxon, a coumaphos, a cyanophos, a diazinon, a dursban, a fensulfothion, a parathion, a methyl parathion, a DEPP, and various phosphodiesters, can be determined by measuring the production of a cleavage product spectrophotometrically at visible and/or UV wavelengths (Dumas, D. P. et al., 1989b). For example, the cleavage of DEPP can be measured at 280 nm, using a spectrophotometer to detect a phenol cleavage product (Watkins, L. M. et al., 1997a; Hong, S.-B. and Raushel, F. M., 1996). In a further example, various phosphodiesters (e.g., an ethyl-4-nitrophenyl phosphate) have been made to evaluate OPH cleavage rates, and their cleavage measured at 280 nm by the production of a substituted phenol cleavage product (Shim, H. et al., 1998). In a further example, a paraoxon is often used to measure OPH activity, because it is both rapidly hydrolyzed by the enzyme and produces a visible cleavage product. To determine kinetic properties, the production of paraoxon's cleavage product, p-nitrophenol, may be measured with a spectrophotometer at 400 nm and/or 420 nm (Dumas, D. P. et al., 1990; Kuo, J. M. and Raushel, F. M., 1994; Watkins, L. M. et al., 1997a; Gopal, S. et al., 2000). In an additional example, a NPPMP cleavage can also be measured by the release of a p-nitrophenol as a cleavage product (diSioudi, B. et al., 1999). In a further example, chiral and non-chiral phosphotriesters have been created to produce a p-nitrophenol as a cleavage product, and thus adapt the method used in measuring a paraoxon cleavage in determining the general binding and/or cleavage preference of an enzyme for a phosphoryl group Sₚ enantiomer, Rₚ enantiomer and/or a non-chiral substrate (Chen-Goodspeed, M. et al., 2001a; Chen-Goodspeed, M. et al., 2001b; Wu, F. et al., 2000a; Steubaut, W. et al., 1975). In an example, chiral sarin and soman analogues have been created wherein the fluoride comprising moiety of the P—F bond has been replaced by p-nitrophenol, allowing detection of the CWA analogs' cleavage rates using the adapted method for paraoxon cleavage measurement (Li, W.-S. et al., 2001).

Other techniques are known in the art for measuring OP detoxification activity, such as, for example, determining the loss of acetylcholinesterase inhibitory potency of an OP compound due to contact with an enzyme (Hoskin, F. C. G., 1990; Luo, C. et al., 1999; Ashani, Y. et al., 1998).

### N. COATINGS

In some embodiments, a material formulation such as a surface treatment (e.g., a coating) comprises a biomolecular composition. Coatings and other surface treatments, and antimicrobial and/or antifouling peptide compositions, enzymes, and their preparation, which may be used in light of the present disclosures have been described in U.S. patent application Ser. Nos. 10/655,345, 10/792,516, and 10/884,355, and provisional patent application 60/711,958, each incorporated by reference).

A coating (“coat,” “surface coat,” “surface coating”) refers to “a liquid, liquefiable or mastic composition that is converted to a solid protective, decorative, or functional adherent film after application as a thin layer” (“Paint and Coating Testing Manual, Fourteenth Edition of the Gardner-Sward Handbook” (Koleske, J. V. Ed.), p. 696, 1995; and in “ASTM Book of Standards, Volume 06.01, Paint—Tests for Chemical, Physical, and Optical Properties; Appearance,” D16-00, 2002). Additionally, a thin layer comprises about 5 um to about 1500 um thick. However, in many embodiments, a coating forms a thin layer about 15 um to about 150 um thick. Examples of a coating include a clear coating or a paint.

However, a material may comprise a layer upon the surface of another material that is thinner, such as from about a molecular layer (e.g., about 32 pm to about 10,000 pm) to about 5 μm thick. Such thinner material layer(s) may be referred to as a “coat,” “coating,” and/or a “film” but are not considered herein to be a coat, coating and/or a film such as in the art of a paint or a clear coating, due to differences such as formulation, preparation, processing, application, function, or a combination thereof. For example, a layer of hydrophobic molecules loosely adhering to a hydrophobic biomolecule may be referred to as a “coat,” “coating,” and/or a “film,” but does not fall into the art of a coating such as a paint applied to a wall.

Examples of such thinner material layers often referred to as a “coat,” “coating,” and/or a “film” includes a molecular scale layer, a microencapsulating material, a seed “coating,” a textile finish, a pharmaceutical encapsulating material, an the like. As used herein and in the claim(s), a coating, a coat, a surface coat, a surface coating, a film, and/or a surface film refers to a coating and/or a coating produced film, as would be understood in the arts of a clear coating and/or a paint, unless otherwise specified in the claims(s) or by the context herein, as would be understood in the respective art(s).

Where the context so indicates, the term “coating” refers to the coating that is applied. For example, a coating may be capable of undergoing a change from a fluent to a nonfluent condition by removal of solvents, vehicles and/or carriers, by setting, by a chemical reaction and/or conversion, and/or by solidification from a molten state. The coating and/or the film that is formed may be hard or soft, elastic or inelastic, permanent or transitory, or a combination thereof. Where the context so indicates, the term “coating” includes the process of applying (e.g., brushing, dipping, spreading, spraying) or otherwise producing a coated surface, which may also be referred to as a coating, coat, covering, film or layer on a surface. Where the context allows, the act of coating also includes impregnating a surface and/or an object by causing a material to extend or penetrate into the object, or into the interstices of a porous, a cellular and/or a foraminous material.

A surface comprises the outer layer of any solid object. The term “substrate,” in the context of a coating, may be synonymous with the term “surface.” However, as “substrate” has a different meaning in the arts of enzymology and coatings, the term “surface” may be preferentially used herein for clarity. A surface wherein a coating has been applied, whether or not film formation has occurred, may be known herein as a “coated surface.”

A coating generally comprises one or more materials that contribute to the properties of the coating, the ability of a coating to be applied to a surface, the ability of the coating to undergo film formation, and/or the properties of the produced film. Examples of such a coating component include a binder, a liquid component, a colorizing agent, an additive, or a combination thereof, and such materials are contemplated for used in a coating. A coating typically comprises a material often referred to as a “binder,” which functions as the primary material in a coating capable of film formation (i.e., producing a film). Often the binder may be the coating component that dominates conferring a physical and/or chemical property to a coating and/or a film. Examples of properties of a binder typically affects include chemical reactivity, minimum film formation temperature, minimum T_{g}, volume fraction solids, a rheological property (e.g., viscosity), film moisture resistance, film UV resistance, film heat resistance, film weathering resistance, adherence, film hardness, film flexibility, or a combination thereof. Consequently, different categories of coatings may be identified herein by the binder used in the coating. For example, a binder may comprise an oil, a chlorinated rubber, and/or an acrylic, and examples of a coating comprising such binders include an oil coating, a chlorinated rubber-topcoat, an acrylic-lacquer, etc. In certain embodiments, a biomolecular composition may function as a binder, particularly in aspects wherein the coating comprises another thermosetting binder that may cross-link to the chemical moiety(s) (e.g., hydroxyl moiety(s), amine moiety(s), polyols, carboxyl moiety(s), fatty acids, double bonds, etc.) typically found in cells.

In many embodiments, a coating may comprise a liquid component (e.g., a solvent, a diluent, a thinner), which often confers and/or alters the coating's rheological properties (e.g., viscosity) to ease the application of the coating to a surface. In some embodiments, a coating may comprise a colorizing agent (e.g., a pigment), which functions to alter an optical property of a coating and/or a film. In particular embodiments, a colorizing agent comprises a biomolecular composition, an extender, a pigment, or a combination thereof. In other embodiments, a coating comprises a colorizing agent comprising a biomolecular composition. A coating may often comprise an additive, which reduces and/or prevents the development of a physical, chemical, and/or aesthetic defect in a coating and/or a film; confers some additional desired property to a coating and/or a film; or a combination thereof. Examples of an additive commonly used in a coating and/or a film include an antifloating agent, an antiflooding agent, an antifoaming agent, a catalyst, a corrosion inhibitor, a dehydrator, an electrical additive, a film-formation promoter, a light stabilizer, a matting agent, a neutralizing agent, a preservative, a rheology modifier, a thickener, a UV stabilizer, a viscosity control agent, a buffer, a viscosity control agent, an accelerator, an adhesion promoter, an antioxidant, an antiskinning agent, a coalescing agent, a defoamer, a dispersant, a drier, an emulsifier, a fire retardant, a flow control agent, a gloss aid, a leveling agent, a marproofing agent, a slip agent, a wetting agent, or a combination thereof. In certain embodiments, a biomolecular composition comprises an additive. In particular embodiments, an additive comprising a biomolecular composition comprises a viscosity control agent, a dispersant, or a combination thereof. In other embodiments, a coating comprises an additive comprising a biomolecular composition. A contaminant comprises a material unintentionally added to a coating, and may comprise volatile and/or non-volatile component of a coating and/or a film. A coating component may be categorized as possessing more than one defining characteristic, and thereby simultaneously functioning in a coating as a combination of a binder, a liquid component, a colorizing agent, and/or an additive. Different coating compositions are described herein as examples of coatings with varying sets of properties.

A coating may be applied to a surface using any technique known in the art. In the context of a coating, “application,” “apply,” or “applying” refers to the process of transferring of a coating to a surface to produce a layer of coating upon the surface. As known herein in the context of a coating, an “applicator” refers to a devise that may be used to apply the coating to a surface. Examples of an applicator include a brush, a roller, a pad, a rag, a spray applicator, etc. Application techniques that are contemplated as suitable for a user of little or no particular skill include, for example, dipping, pouring, siphoning, brushing, rolling, padding, ragging, spraying, etc. Certain types of coatings may be applied using techniques contemplated as more suitable for a skilled artisan such as anodizing, electroplating, and/or laminating of a film onto a surface.

In certain embodiments, the layer of coating undergoes film formation (“curing,” “cure”), which refers to the physical and/or chemical change of a coating to a solid when in the form of a layer upon the surface. In certain aspects, a coating may be prepared, applied and cured at an ambient condition, a baking condition, or a combination thereof. An ambient condition comprises a temperature range between about −10° C. to about 40° C. (e.g., contacting the material formulation with a material such as a solid, liquid, air; IR irradiation, etc). As used herein, a “baking condition” or “baking” comprises contacting a material formulation with a temperature (e.g., heated air, liquid, solid, IR irradiation, etc.) above about 40° C. and/or raising the temperature of a material formulation above about 40° C., typically to promote film formation. For example, baking a coating include contacting a coating with a material at a baking temperature and/or raising the temperature of coating to about 40° C. to about 300° C., or more. Various coatings, for example, may be applied and/or cured at ambient conditions, baking conditions, or a combination thereof.

In general embodiments, a coating comprising a biomolecular composition may be prepared, applied and cured at any temperature range described herein and/or may be applicable in the art in light of the present disclosures. An example of such a temperature range comprises about −100° C. to about 300° C., or more. However, a biomolecular composition material may further comprise a desired biomolecule (e.g., a colorant, an enzyme, a peptide), whether endogenously and/or recombinantly produced, that may have a reduced tolerance to temperature. The temperature that may be tolerated by a biomolecule may vary depending on the specific biomolecule used in a coating, and may generally be within the range of temperatures tolerated by the living organism from which the biomolecule was derived. For example, a coating comprising a biomolecular composition, wherein the biomolecular composition comprises an enzyme, that the coating may be prepared, applied and cured at about −100° C. to about 110° C. For example, a temperature of about −100° C. to about 40° C. may be suitable for many enzymes (e.g., a wild-type sequence and/or a functional equivalent) derived from an eukaryote, while temperatures up to, for example about −100° C. to about 50° C. may be tolerated by enzymes derived from many prokaryotes.

The type of film formation that a coating may undergo depends upon the coating components. A coating may comprise, for example, a volatile coating component, a non-volatile coating component, or a combination thereof. In certain aspects, the physical process of film formation comprises loss of about 1% to about 100%, of a volatile coating component. In general embodiments, a volatile component may be lost by evaporation. In certain aspects, loss of a volatile coating component during film formation reaction may be promoted by baking the coating. Examples of a volatile coating component include a coalescing agent, a solvent, a thinner, a diluent, or a combination thereof. A non-volatile component of the coating remains upon the surface. In specific aspects, the non-volatile component forms a film. Examples of non-volatile coating components include a binder, a colorizing agent, a plasticizer, a coating additive, or a combination thereof. A non-volatile coating component may comprise a cell-based particulate material. In specific aspects, a coating component may undergo a chemical change to form a film. In general embodiments, a binder undergoes a cross-linking and/or a polymerization reaction to produce a film. In general embodiments, a chemical film formation reaction occurs spontaneously under ambient conditions. In other aspects, a chemical film formation reaction may be promoted by irradiating the coating, heating the coating, or a combination thereof. In some embodiments, irradiating the coating comprises exposing the coating to electromagnetic radiation, particle radiation, or a combination thereof. Examples of electromagnetic radiation used to irradiate a coating include UV radiation, infrared radiation, or a combination thereof. Examples of particle radiation used to irradiate a coating include electron-beam radiation. Often irradiating the coating induces an oxidative and/or free radical chemical reaction that cross-links of one or more coating components.

However, in some alternate embodiments, a coating undergoes a reduced amount of film formation than such a solid film is not produced, or does not undergo film formation to a measurable extent during the period of time it may be used on a surface. Such a coating may be referred to herein as a “non-film forming coating.” Such a non-film forming coating may be prepared, for example, by increasing the non-volatile component in a thermoplastic coating (e.g., increasing plasticizer content in a liquid component), reducing the amount of a coating component that contributes to the film formation chemical reaction (e.g., a binder, a catalyst), increasing the concentration of a component that inhibits film formation (e.g., an antioxidant/radical scavenger in an oxidation/radical cured thermosetting coating), reducing the contact with an external a curing agent (e.g., radiation, baking), selection of a non-film formation binder produced from component(s) that lack cross-linking moiety(s), selection of a non-film formation binder that lack sufficient size to undergo thermoplastic film formation, or a combination thereof. As used herein, a “non-film formation binder” refers to a molecule that may be chemically similar to a binder, but lacks sufficient size, a cross-linking moiety, and/or a polymerization moiety to undergo film formation. For example, a coating may be prepared by selection of an oil-based binder that lacks sufficient double bonds to undergo sufficient cross-linking reactions to produce a film. In another example, a non-film formation binder may be selected that lacks sufficient cross-linking moiety(s) such as an epoxide, an isocyanate, a hydroxyl, a carboxyl, an amine, an amide, a silicon moiety, etc., to produce a film by thermosetting. Such a non-film formation binder may be prepared by chemical modification of a binder, such as, for example, a cross-linking reaction with a small molecule (e.g., less than 1 kDa) comprising a moiety capable of reaction with a binder's cross-linking moiety, to produce a chemically blocked binder moiety inert to a further cross-linking reaction. In another example, a thermoplastic binder typically comprises a molecule 29 kDa to 1000 kDa or more in size, though more specific, ranges for different binders (e.g., an acrylic, a polyvinyl, etc.) are described herein. Film formation may be reduced or prevented by selection of a like molecule too small to effectively undergo thermoplastic film formation. An example includes selection of a non-film formation binder molecule between 1 kDa to 29 kDa in molecular weight.

In other alternative embodiments, a coating may undergo film formation, but produce a film whose properties makes it more suited for a temporary use. Such a temporary film may possess a poor and/or low rating for a property that may confer longevity in use. For example, a film with a poor abrasion (e.g., scrub) resistance, a poor solvent resistance, a poor water resistance, a poor weathering property (e.g., UV resistance), a poor adhesion property, a poor microorganism/biological resistance, or a combination thereof, may be selected as a temporary film. Such a “poor” or “low” property may be determined by standards in the art, and often the detection of the coating property (e.g., a change in the coating's color, gloss, loss of coating material) and/or may be a rating in the half of a standard test rating scale and/or a detectable property associated with a reduced longevity of use. In one aspect, a film may have poor adhesion for a surface, allowing ease of removal by stripping and/or peeling. In certain aspects, a poor or low adhesion rating on a scale of 0 (lowest adhesion) to 5 may be denoted 2A, 1A, 0A, 2B, 1B, 0B, as described in “ASTM Book of Standards, Volume 06.01, Paint—Tests for Chemical, Physical, and Optical Properties; Appearance,” D3359-97, 2002. Other examples of standard adhesion assays that may be used to determine a poor or low adhesion property rating include “ASTM Book of Standards, Volume 06.01, Paint—Tests for Chemical, Physical, and Optical Properties; Appearance,” D5179-98 and D2197-98, 2002; “ASTM Book of Standards, Volume 06.02, Paint—Products and Applications; Protective Coatings; Pipeline Coatings,” D4541-02, D3730-98, D4145-83, D4146-96, and D6677-01, 2002; and “ASTM Book of Standards, Volume 06.02, Paint—Products and Applications; Protective Coatings; Pipeline Coatings,” D5064-01, 2002. In other aspects, a poor or low abrasion rating for a coating may be denoted as a detectable gloss, color and/or material erosion, such as an increase (“I”), large increase (“Li”), decrease (“D”), or large decrease (“LD”) gloss change, a slightly darker (“SD”), considerably darker (“CD”), slightly lighter (“SL”) or considerably lighter (“CL”) color change, a slight (“S”) or moderate (“M”) erosion change, for gloss, color and/or erosion, as described in “ASTM Book of Standards, and Volume 06.02, Paint—Products and Applications; Protective Coatings; Pipeline Coatings,” D4828-94, 2002. Additional examples of standard abrasion tests that may be used to determine a poor or low abrasion resistance property rating include those described in “ASTM Book of Standards, Volume 06.01, Paint—Tests for Chemical, Physical, and Optical Properties; Appearance,” D968-93 and D4060-01, 2002; and “ASTM Book of Standards, and Volume 06.02, Paint—Products and Applications; Protective Coatings; Pipeline Coatings,” D3170-01, D4213-96, D2486-00, D3450-00, D6736-01, and D6279-99e1, 2002. Weathering resistance may be described in “ASTM Book of Standards, Volume 06.01, Paint—Tests for Chemical, Physical, and Optical Properties; Appearance,” D4141-01, D1729-96, D660-93, D661-93, D662-93, D772-86, D4214-98, D3274-95, D714-02, D1654-92, D2244-02, D523-89, D1006-01, D1014-95, and D1186-01, 2002; “ASTM Book of Standards, Volume 06.02, Paint—Products and Applications; Protective Coatings; Pipeline Coatings,” D3719-00, D610-01, D1641-97, D2830-96, and D6763-02, 2002; and “ASTM Book of Standards, Volume 06.01, Paint—Tests for Chemical, Physical, and Optical Properties; Appearance,” D822-01, D4587-01, D5031-01, D6631-01, D6695-01, D5894-96, and D4141-01, 2002; “ASTM Book of Standards, Volume 06.02, Paint—Products and Applications; Protective Coatings; Pipeline Coatings,” D5722-95, D3361-01 and D3424-01, 2002. Examples of poor weathering resistance includes a blistering rating of dense (“D”), medium dense (“MD”), medium (“M”) blistering, a failure at scribe, which comprises a measure of corrosion and paint loss at the site of contact with a tool known as a scribe, in the range of 0 to 5, a rating of the unscribed areas of 0 to 5, a rust grade rating of a coated steel surface of 0 to 5, a general appearance rating of 0 to 5, a cracking rating of 0 to 5, a checking rating of 0 to 5, a dulling rating of 0 to 5, and/or a discoloration rating of 0 to 5, respectively, as described in “ASTM Book of Standards, Volume 06.01, Paint—Tests for Chemical, Physical, and Optical Properties; Appearance,” D714-02and D1654-92, 2002; and “ASTM Book of Standards, Volume 06.02, Paint—Products and Applications; Protective Coatings; Pipeline Coatings,” D610-01 and D1641-97, 2002. In additional aspects, a poor or low solvent resistance rating for a coating may be denoted as a solvent resistance rating of 0 to 2, a coating removal efficiency rating of 3 to 5, an effect of coating removal on the condition of the surface of 0 to 2, respectively, as described in “ASTM Book of Standards, Volume 06.02, Paint—Products and Applications; Protective Coatings; Pipeline Coatings,” D4752-98, 2002; and “ASTM Book of Standards, Volume 06.02, Paint—Products and Applications; Protective Coatings; Pipeline Coatings,” D6189-97, 2002. An additional example of a standard solvent resistance assay may be described in “ASTM Book of Standards, Volume 06.02, Paint—Products and Applications; Protective Coatings; Pipeline Coatings,” D5402-93, 2002. In further aspects, a poor or low water resistance rating for a coating may be denoted as a discernable change in a coating's color, blistering, adhesion, softening, and/or embrittlement upon conducting an assay as described in “ASTM Book of Standards, Volume 06.01, Paint—Tests for Chemical, Physical, and Optical Properties; Appearance,” D2247-02 and D4585-99, 2002. Further assays for water resistance are described in “ASTM Book of Standards, Volume 06.01, Paint—Tests for Chemical, Physical, and Optical Properties; Appearance,” D870-02, D1653-93, D1735-02, 2002; and “ASTM Book of Standards, Volume 06.02, Paint—Products and Applications; Protective Coatings; Pipeline Coatings,” D2065-96, D2921-98, D3459-98, and D6665-01, 2002.

In particular aspects, growth of cells, particularly microorganisms, may produce a coating and/or a film with reduced stability, film formation capability, durability, etc. Such a non-film formatting film and/or a temporary film may be prepared by the inclusion of the cell-based particulate material, particularly in embodiments wherein the cell-based particulate material comprises a non-sterilized cell-based particulate material; the coating has a reduced concentration of biocide such as about 0% to about 99.9999%, a typically used concentration for a coating comprising the cell-based particulate material; the coating comprises a nutrient (e.g., a cell-based particulate material, other digestible material, vitamins, trace minerals, etc.) as a coating component (e.g., an additive) that promotes cell growth; or a combination thereof.

In additional aspects, a poor and/or a low microorganism/biological resistance rating for a coating may be denoted as a colony recovery/growth rating of 2 to 4, a discoloration/disfigurement rating of 0 to 5, a fouling resistance (“F.R.”) or antifouling film (“A.F”) rating of 0 to 70, and observed growth (e.g., fungal growth) on specimens of 2 to 4, respectively, as described in “ASTM Book of Standards, Volume 06.01, Paint—Tests for Chemical, Physical, and Optical Properties; Appearance,” D3274-95, D2574-00, D3273-00, D5589-97 and D5590-00, 2002; and in “ASTM Book of Standards, Volume 06.02, Paint—Products and Applications; Protective Coatings; Pipeline Coatings,” D3623-78a, 2002. An additional example of a standard microorganism/biological resistance assay may be described in “ASTM Book of Standards, Volume 06.01, Paint—Tests for Chemical, Physical, and Optical Properties; Appearance,” D4610-98 and D3456-86, 2002; in “ASTM Book of Standards, Volume 06.02, Paint—Products and Applications; Protective Coatings; Pipeline Coatings,” D4938-89, D4939-89, D5108-90, D5479-94, D6442-99, D6632-01, D4940-98 and D5618-94, 2002; and “ASTM Book of Standards, Volume 06.03, Paint—Pigments, Drying Oils, Polymers, Resins, Naval Stores, Cellulosic Esters, and Ink Vehicles,” D912-81 and D964-65, 2002.

In another example, a film may have a poor resistance to an environmental factor, and subsequently fail (e.g., crack, peel, chalk, etc.) to remain a viable film upon the surface. For example, a film may undergo chalking. Chalking refers to the erosion a coating, typically by degradation of the binder due to various environmental forces (e.g., UV irradiation). In some embodiments, chalking may be used to remove a contaminant from the surface of a film and/or expose a component of the film (e.g., a biomolecular composition) to the surface of the film. In some aspects, a chalking coating has a chalking rating on a “Wet Finger Method” of visible or severe and a chalk reflectance rating of 0 to 5, as described in “ASTM Book of Standards, Volume 06.01, Paint—Tests for Chemical, Physical, and Optical Properties; Appearance,” D4214-98, 2002. A self-cleaning coating comprises a film with a high chalking property. In many aspects the layer of non-film forming coating, a temporary film and/or a self-cleaning film may be removed from a surface with ease. In such embodiments, a non-film forming coating, a temporary film, a self-cleaning film, or a combination thereof may be more suitable for a temporary use upon a surface, due to the ability to be applied as a layer and easily removed when its presence no longer desired. In these embodiments, the non-film forming coating, the temporary film, the self-cleaning film, or a combination thereof, may be desired for a use upon a surface that lasts a temporary period of time, such as, for example, about 1 to about 60 seconds, about 1 to about 24 hours, about 1 to about 7 days, about 1 to about 10 weeks, about 1 to about 6 months, respectively.

In some embodiments, a plurality of coating layers, known herein as a “multicoat system” (“multicoating system”), may be applied upon a surface. The coating selected for use in a specific layer may differ from an additional layer of the multicoat system. This selection of coatings with differing components and/or properties may be done to sequentially confer, in a desired pattern, the properties of differing coatings to a coated surface and/or multicoat system. Examples of a coating that may be selected for use, either alone or in a multicoat system, include a sealer, a water repellent, a primer, an undercoat, a topcoat, or a combination thereof. A sealer comprises a coating applied to a surface to reduce or prevent absorption by the surface of a subsequent coating layer and/or a coating component thereof, and/or to prevent damage to the subsequent coating layer by the surface. A water repellant comprises a coating applied to a surface to repel water. A primer comprises a coating applied to increase adhesion between the surface and a subsequent layer. In typical embodiments a primer-coating, a sealer-coating, a water repellent-coating, or a combination thereof, may be applied to a porous surface. Examples of a porous surface include a drywall, a wood, a plaster, a masonry, a damaged film, a degraded film, a corroded metal, or a combination thereof. In certain aspects, the porous surface may be not coated and/or lacks a film prior to application of a primer, a sealer, a water repellent, or a combination thereof. An undercoat comprises a coating applied to a surface to provide a smooth surface for a subsequent coat. A topcoat (“finish”) comprises a coating applied to a surface for a protective and/or a decorative purpose. Of course, a sealer, a water repellent, a primer, an undercoat, and/or a topcoat may possess additional protective, decorative, and/or functional properties. Additionally, the surface a sealer, a water repellent, a primer, an undercoat, and/or a topcoat may be applied to a coated surface such as a coating and/or a film of a layer of a multicoat system. In certain embodiments, a multicoat system may comprise any combination of a sealer, a water repellent, a primer, an undercoat, and/or a topcoat. For example, a multicoat system may comprise any of the following combinations: a sealer, a primer and a topcoat; a primer and a topcoat; a water repellent, a primer, an undercoat, and a topcoat; an undercoat and a topcoat; a sealer, an undercoat, and a topcoat; a sealer and a topcoat; a water repellent and a topcoat, etc. In particular aspects, a coating layer may comprise properties that may comprise a combination of those associated with different coating types such as a sealer, a water repellent, a primer, an undercoat, and/or a topcoat. In such instances, such a combination coating and/or film may be designated by a backslash “/” separating the individual coating designations encompassed by the layer.

Examples of such a coating layer comprising a plurality of functions include a sealer/primer coating, a sealer/primer/undercoat coating, a sealer/undercoat coating, a primer/undercoat coating, a water repellant/primer coating, an undercoat/topcoat coating, a primer/topcoat coating, a primer/undercoat/topcoat coating, etc. In embodiments wherein the coated surface comprises a particular type of coating, then the coated surface may be known herein by the type of coating such as a “painted surface,” a “clear coated surface,” a “lacquered surface,” a “varnished surface,” a “water repellant/primered surface,” an “primer/undercoat-topcoated surface,” etc.

In specific aspects, a multicoat system may comprise a plurality of layers of the same type, such as, for example, about 1 to about 10 layers, of a sealer, a water repellent, a primer, an undercoat, a topcoat, or a combination thereof. In specific facets, a multicoat system comprises a plurality of layers of the same coating type, such as, for example, about 1 to about 10 layers, of a sealer, a water repellent, a primer, an undercoat, and/or a topcoat. In embodiment where a coating does not comprise a multicoat system, but a single layer of coating applied to a surface, such a layer, regardless of typical function in a multicoat system, may be regarded herein as a topcoat.

1. Paints

A paint generally refers to a “pigmented liquid, liquefiable or mastic composition designed for application to a substrate in a thin layer which is converted to an opaque solid film after application. Used for protection, decoration or identification, or to serve some functional purpose such as the filling or concealing of surface irregularities, the modification of light and heat radiation characteristics, etc.” [“Paint and Coating Testing Manual, Fourteenth Edition of the Gardner-Sward Handbook” (Koleske, J. V. Ed.), p. 696, 1995]. However, as certain coatings disclosed herein are non-film forming coatings, this definition is modified herein to encompass a coating with the same properties of a film forming paint, with the exception that it does not produce a solid film. In particular embodiments, a non-film forming paint possesses a hiding power sufficient to concealing surface feature comparable to an opaque film.

Hiding power refers to the ability of a coating and/or a film to prevent light from being reflected from a surface, particularly to convey the surface's visual pattern. Opacity refers to the hiding power of a film. An example of hiding power comprises the ability of a paint-coating to visually block the appearance of grain and color of a wooden surface, as opposed to a clear varnish-coating allowing the relatively unobstructed appearance of wood to pass through the coating. Standard techniques for determining the hiding power of a coating and/or a film (e.g., paint, a powder coating) are described, for example, in “ASTM Book of Standards, Volume 06.01, Paint—Tests for Chemical, Physical, and Optical Properties; Appearance,” E284-02b, D344-97, D2805-96a, D2745-00 and D6762-02a 2002; “ASTM Book of Standards, Volume 06.02, Paint—Products and Applications; Protective Coatings; Pipeline Coatings,” D5007-99, D5150-92 and D6441-99, 2002; and “Paint and Coating Testing Manual, Fourteenth Edition of the Gardner-Sward Handbook” (Koleske, J. V. Ed.), pp. 481-506, 1995.

2. Clear-Coatings

A clear-coating refers to a coating that is not opaque and/or does not produce an opaque solid film after application. A clear-coating and/or film may be transparent or semi-transparent (e.g., translucent). A clear-coating may be colored or non-colored. In certain embodiments, reducing the content of a pigment in a paint composition may produce a clear-coating. Additionally, a clear-coating may comprise a lacquer, a varnish, a shellac, a stain, a water repellent coating, or a combination thereof. Though some opaque coatings are referred to in the art as a lacquer, a varnish, a shellac, or a water repellent coating, all such opaque coatings are considered as paints herein (e.g., a lacquer-paint, a varnish-paint, a shellac-paint, a water repellent paint).

a. Varnishes

A varnish comprises a thermosetting coating that converts to a transparent or translucent solid film after application. In general embodiments, a varnish comprises a wood-coating. A varnish comprises an oil and a dissolved binder. In general embodiments, the oil comprises a drying oil, wherein the drying oil functions as an additional binder. In other embodiments, the binder may be solid at ambient conditions prior to dissolving into the oil and/or an additional liquid component of the varnish. Examples of a dissolvable binder include a resin obtained from a natural source (e.g., a Congo resin, a copal resin, a damar resin, a kauri resin), a synthetic resin, or a combination thereof. In specific aspects, the additional liquid component comprises a solvent such as a hydrocarbon solvent. In some facets, the solvent may be added to reduce viscosity of the varnish. A varnish may further comprise a coloring agent, including a pigment, for such purposes as conferring and/or altering a color, a gloss, a sheen, or a combination thereof. A varnish undergoes thermosetting film formation by oxidative cross-linking. In certain aspects, a varnish may additionally undergo film-formation by evaporation of a volatile component. The dissolved binder generally functions to shorten the time to film-formation relative to certain measures (e.g., dryness, hardness), though the final cross-linking reaction time may not be significantly and/or measurably shortened. Standards for determining a varnish-coating and/or film's properties are described in, for example, “ASTM Book of Standards, Volume 06.03, Paint—Pigments, Drying Oils, Polymers, Resins, Naval Stores, Cellulosic Esters, and Ink Vehicles,” D154-85, 2002.

b. Lacquers

A lacquer comprises a thermoplastic, solvent-borne coating that converts to a transparent or translucent solid film after application. In general embodiments, a lacquer comprises a wood-coating. A lacquer-coating comprises a thermoplastic binder dissolved in a liquid component comprising an active solvent. Examples of a thermoplastic binder include a cellulosic binder (e.g., a nitrocellulose, a cellulose acetate), a synthetic resin (e.g., an acrylic), or a combination thereof. In certain aspects, a liquid component comprises an active solvent, a latent solvent, diluent, a thinner, or a combination thereof. In certain embodiments, a lacquer comprises a nonaqueous dispersion (“NAD”) lacquer, wherein the content of solvent may be not sufficient to fully dissolve the thermoplastic binder. In certain aspects, a lacquer may comprise an additional binder (e.g., an alkyd), a colorant, a plasticizer, or a combination thereof. Film formation of a lacquer occurs by loss of the volatile component(s), typically through evaporation.

Standards for a lacquer-coating and/or a film's composition (e.g., a lacquer, a pigmented-lacquer, a nitrocellulose lacquer, a nitrocellulose-alkyd lacquer), physical and/or chemical properties (e.g., heat and cold resistance, hardness, film-formation time, stain resistance, particulate material dispersion), and procedures for testing a lacquer's composition/properties, are described in, for example, in “ASTM Book of Standards, Volume 06.02, Paint—Products and Applications; Protective Coatings; Pipeline Coatings,” D333-01, D2337-01, D3133-01, D365-01, D2091-96, D2198-02, D2199-82, D2571-95 and D2338-02, 2002.

c. Shellacs

A shellac may be similar to a lacquer, but the binder does not comprise a nitrocellulose binder, and the binder may be soluble in alcohol, and the binder may be obtained from a natural source. In some embodiments, a binder comprises Laciffer lacca beetle secretion. In general embodiments, a shellac comprises a liquid component (e.g., alcohol). In specific aspects, the additional liquid component comprises a solvent. In some facets, the liquid component may be added to reduce viscosity of the varnish. In other embodiments, a shellac undergoes rapid film formation. Standards for a shellac-coating and/or film's composition and properties are described in, for example, “ASTM Book of Standards, Volume 06.03, Paint—Pigments, Drying Oils, Polymers, Resins, Naval Stores, Cellulosic Esters, and Ink Vehicles,” D29-98 and D360-89, 2002.

d. Stains

A stain comprises a clear or semitransparent coating formulated to change the color of surface. In general embodiments, a stain comprises a wood-coating designed to color and/or protect a wood surface but not conceal the grain pattern and/or texture. A stain comprises a binder such as an oil, an alkyd, or a combination thereof. Often a stain comprises a low solid content. A low solids content for a wood stain may be less than about 20% volume of solids. The low solid content of a stain promotes the ability of the coating to penetrate the material of the wooden surface. This property may be used to, for example, to promote the incorporation of a fungicide that may be comprised within the stain into the wood. In certain alternative aspects, a stain comprises a high solids content stain, wherein the solid content may be about 20% or greater, may be used on a surface to produce a film possessing the property of little or no flaking. In other alternative aspects, a water-borne stain may be used such as a stain comprising a water-borne alkyd. A stain typically further comprises a liquid component (e.g., a solvent), a fungicide, a pigment, or a combination thereof. In other aspects, a stain comprises a water repellent hydrophobic compound so it functions as a water repellent-coating (“stain/water repellent-coating”). Examples of a water repellent hydrophobic compound a stain may comprise include a silicone oil, a wax, or a combination thereof. Examples of a fungicide include a copper soap, a zinc soap, or a combination thereof. Examples of a pigment include a pigment that may be similar in color to wood. Examples of such a pigment includes a red pigment (e.g., a red iron oxide) a yellow pigment (e.g., a yellow iron oxide), or a combination thereof. Standards procedures for testing a stain's (e.g., an exterior stain) properties, are described in, for example, in “ASTM Book of Standards, Volume 06.02, Paint—Products and Applications; Protective Coatings; Pipeline Coatings,” D6763-02, 2002.

e. Water Repellent-Coatings

A water repellent-coating comprises a coating comprising hydrophobic compounds that repel water. A water repellent-coating may be applied to a surface susceptible to water damage, such as a metal, a masonry, a wood, or a combination thereof. A water repellent-coating typically comprises a hydrophobic compound and a liquid component. In specific embodiments, a water repellent-coating comprises about 1% to about 65% hydrophobic compound. Examples of a hydrophobic compound that may be selected include an acrylic, a siliconate, a metal-searate, a silane, a siloxane, a parafinnic wax, or a combination thereof. A water repellent coating may comprise a water-borne coating and/or a solvent-borne coating. A solvent-borne water repellent-coating typically comprises a solvent that dissolves the hydrophobic compound. Examples of such a solvent includes an aliphatic, an aromatic, a chlorinated solvent, or a combination thereof.

In certain embodiments, a water repellent-coating undergoes film formation, penetrates pores, or a combination thereof. In certain aspects, an acrylic-coating, a silicone-coating, or a combination thereof, undergoes film formation. In other aspects, a metal-searate, a silane, a siloxane, a parafinnic wax, or a combination thereof, penetrates pores in a surface. In some facets, a water repellent-coating (e.g., a silane, a siloxane) covalently bonds to a surface and/or a pore (e.g., masonry). Standards for a water repellent-coating and/or film's composition and properties are described in, for example, “ASTM Book of Standards, Volume 06.02, Paint—Products and Applications; Protective Coatings; Pipeline Coatings,” D2921-98, 2002; and in “Paint and Coating Testing Manual, Fourteenth Edition of the Gardner-Sward Handbook,” (Koleske, J. V. Ed.), pp. 748-750, 1995. Alternatively, standards for a sealer-coating (e.g., a floor sealer) and/or a film's composition and properties are described in, for example, “ASTM Book of Standards, Volume 06.02, Paint—Products and Applications; Protective Coatings; Pipeline Coatings,” D1546-96, 2002;

3. Coating Categories by Use

In light of the present disclosures, a coating may be prepared and applied to any surface. However, the coating components and methods described herein are selected for a particular application to provide a coating and/or a film with properties suited for a particular use. For example, a coating used in an external environment may comprise a coating component of improved UV resistance than a coating used in an interior environment. In another example, a film used upon a surface of a washing machine may comprise a component that confers improved moisture resistance than a component of a film for use upon a ceiling surface. In a further example, a coating applied to the surface of an assembly line manufactured product may comprise components suitable for application by a spray applicator. Various properties of coating components are described herein to provide guidance to the selection of specific coating compositions with a suitable set of properties for a particular use.

A coating may be classified by its end use, including, for example, as an architectural coating, an industrial coating, a specification coating, or a combination thereof. An architectural coating refers to “an organic coating intended for on-site application to interior or exterior surfaces of residential, commercial, institutional, or industrial buildings, in contrast to industrial coatings. They are protective and decorative finishes applied at ambient conditions” [“Paint and Coating Testing Manual, Fourteenth Edition of the Gardner-Sward Handbook” (Koleske, J. V. Ed.), p. 686, 1995)]. An industrial coating refers to a coating applied in a factory setting, typically for a protective and/or aesthetic purpose. A specification coating (“specification finish coating”) refers to a coating formulated to a “precise statement of a set of requirements to be satisfied by a material, produce, system, or service that indicates the procedures for determining whether each of the requirements are satisfied” [“Paint and Coating Testing Manual, Fourteenth Edition of the Gardner-Sward Handbook” (Koleske, J. V. Ed.), p. 891, 1995]. Often, a coating may be categorized as a combination of an architectural coating, an industrial coating, and/or a specification coating. For example, a coating for the metal surfaces of ships may be classified as specification coating, as specific criteria of water resistance and corrosion resistance are required in the film, but typically such a coating may be classified as an industrial coating, since it would typically be applied in a factory. Various examples of an architectural coating, an industrial coating and/or a specification coating and coating components are described herein. Additionally, architectural coatings, industrial coatings, specification coatings examples are described, for example, in “Paint and Surface Coatings: Theory and Practice” 2^{nd} Edition, pp. 190-192, 1999; in “Paints, Coatings and Solvents” 2^{nd} Edition, pp. 330-410, 1998; in “Organic Coatings: Science and Technology, Volume 1: Film Formation, Components, and Appearance” 2^{nd} Edition, pp. 138 and 317-318.

a. Architectural Coatings

An architectural coating (“trade sale coating,” “building coating,” “decorative coating,” “house coating”) comprises a coating suitable to coat surface materials commonly found as part of buildings and/or associated objects (e.g., furniture). Examples of a surface an architectural coating may be applied to include, a plaster surface, a wood surface, a metal surface, a composite particle board surface, a plastic surface, a coated surface (e.g., a painted surface), a masonry surface, a floor, a wall, a ceiling, a roof, or a combination thereof. Additionally, an architectural coating may be applied to an interior surface, an exterior surface, or a combination thereof. An interior coating generally possesses properties such as minimal odor (e.g., no odor, very low VOC), good blocking resistance, print resistance, good washability (e.g., wet abrasion resistance), or a combination thereof. An exterior coating may be selected to possess good weathering properties. Examples of coating type commonly used as an architectural coating include an acrylic-coating, an alkyd-coating, a vinyl-coating, a urethane-coating, or a combination thereof. In certain aspects, a urethane-coating may be applied to a piece of furniture. In other facets, an epoxy-coating, a urethane-coating, or a combination thereof, may be applied to a floor. In some embodiments, an architectural coating comprises a multicoat system. In certain aspects, an architectural coating comprises a high performance architectural coating (“HIPAC”). A HIPAC produces a film with a combination of good abrasion resistance, staining resistance, chemical resistance, detergent resistance, and mildew resistance. Examples of binders suitable for producing a HIPAC include a two-pack epoxide, a two-pack urethane, and/or a moisture cured urethane. In general embodiments, an architectural coating comprises a liquid component, an additive, or a combination thereof. In certain aspects, an architectural coating comprises a water-borne coating and/or a solvent-borne coating. In other aspects, an architectural coating comprises a pigment. In some aspects, such an architectural coating may be formulated to comprise a reduced amount or lack a toxic coating component. Examples of a toxic coating component include a heavy metal (e.g., lead), a formaldehyde, a nonyl phenol ethoxylate surfactant, a crystalline silicate, or a combination thereof.

In certain embodiments, a water-borne coating has a density of about 1.20 Kg/L to about 1.50 Kg/L. In other embodiments, a solvent-borne coating has a density of about 0.90 Kg/L to about 1.2 Kg/L. The density of a coating may be empirically determined, for example, as described in “ASTM Book of Standards, Volume 06.01, Paint—Tests for Chemical, Physical, and Optical Properties; Appearance,” D1475-98, 2002. In certain embodiments, a course particle content of an architectural coating, by weight, may comprise about 0.5% to about 0%. A coarse particle (e.g., a coarse contaminant, a pigment agglomerate) content of a coating may be empirically determined, for example, as described in “ASTM Book of Standards, Volume 06.03, Paint—Pigments, Drying Oils, Polymers, Resins, Naval Stores, Cellulosic Esters, and Ink Vehicles,” D185-84, 2002. In some embodiments, the viscosity for an architectural coating at relatively low shear rates used during typical application, in Krebs Units (“Ku”), may comprise about 72Ku to about 95Ku.

In typical use, an architectural coating may be stored in a container for day(s), month(s) and/or year(s) prior to first use, and/or between different uses. In many embodiments, an architectural coating may retain a set properties of a coating, film formation, a film, or a combination thereof, for a period of 12 months or greater in a container at ambient conditions. Properties that are contemplated for storage include settling resistance, skinning resistance, coagulation resistance, viscosity alteration resistance, or a combination thereof. Storage properties may be empirically determined for a coating (e.g., an architectural coating) as described, for example, in “ASTM Book of Standards, Volume 06.02, Paint—Products and Applications; Protective Coatings; Pipeline Coatings,” D869-85 and D1849-95, 2002.

Application and/or film formation of an architectural coating may occur at ambient conditions to provide ease of use to a casual user of the coating, as well as reduce potential damage to the target surface and the surrounding environment (e.g., unprotected people and objects). In many embodiments, an architectural coating does not undergo film formation by a temperature greater than about 40° C. to reduce possible heat and fire damage. In other embodiments, an architectural coating may be suitable to be applied by using hand-held applicator. Hand-held applicators are generally used without difficulty by many users of a coating, and examples include a brush, a roller, a sprayer (e.g., a spray can), or a combination thereof.

Specific procedures for determining the suitability of a coating and/or a film for use as an architectural coating (e.g., a water-borne coating, a solvent-borne coating, an interior coating, an exterior paint, a latex paint), and specific assays for properties typically desired in an architectural coating (e.g., blocking resistance, hiding power, print resistance, washability, weatherability, corrosion resistance) have been described, for example, in “ASTM Book of Standards, Volume 06.02, Paint—Products and Applications; Protective Coatings; Pipeline Coatings,” D5324-98, D5146-98, D3730-98, D1848-88, D5150-92, D2064-91, D4946-89, D6583-00, D3258-00, and D3450-00, 2002; “ASTM Book of Standards, Volume 06.01, Paint—Tests for Chemical, Physical, and Optical Properties; Appearance,” D660-93, D4214-98, D772-86, D662-93, and D661-93, 2002; and in “Paint and Coating Testing Manual, Fourteenth Edition of the Gardner-Sward Handbook” (Koleske, J. V. Ed.), pp. 696-705, 1995.

i. Wood Coatings

A wood coating may be selected to protect the wood from damage and/or an aesthetic purpose. For example, wood may be susceptible to damage from a bacteria and/or a fungi. Examples of a fungi that damage wood include an Aureobasidium pullulans, an Ascomycotina, a Deutermycotina, a Basidiomycetes, a Coniophora puteana, a Serpula lacrymans, and/or a Dacrymyces stillatus. In some embodiments, a wooden surface may be impregnated with a preservative such as a fungicide, prior to application of a coating. However, much of the wood surface for a coating may be provided this way from wood suppliers. Specific procedures for determining the presence of a preservative and/or water repellent in wood have been described, for example, in “ASTM Book of Standards, Volume 06.02, Paint—Products and Applications; Protective Coatings; Pipeline Coatings,” D2921-98, 2002.

Typically, wood surfaces are coated with a paint, a varnish, a stain, or a combination thereof. Often, the choice of coating may be based on the ability of a coating to protect the wood from damage by moisture. Generally, a paint, a varnish, and a stain generally have progressively greater permeability to moisture, and moisture penetration of a wooden surface which may lead to alterations in wood structure (e.g., splitting); alteration in piece of wood's dimension (“dimensional movement”) such as shrinking, swelling, and/or warping; promote the growth of a microorganism such as fungi (e.g., wet rot, dry rot); or a combination thereof. Additionally, UV light irradiation damages a wood surface by depolymerizing lignin comprised in the wood. In embodiments wherein a wood surface may be irradiated by UV light (e.g., sunlight), the wood coating comprises a UV protective agent such as a pigment that absorbs UV light. An example of a UV absorbing pigment includes a transparent iron oxide.

In specific embodiments, a paint for use on a wood surface comprises an oil-paint, an alkyd-paint, or a combination thereof. A type of alkyd-paint for use on a wood surface comprises a solvent-borne paint. In some embodiments, a paint system comprises a combination of a primer, an undercoat, and a topcoat. A film produced by a paint may be moisture impermeable. A film produced by paint upon a wooden surface may crack, flake, trap moisture that may encourage wood decay, be expensive to repair, or a combination thereof.

ii. Masonry Coatings

Masonry coatings refer to coatings used on a masonry surface, such as, for example, a stone, a brick, a tile, a cement-based material (e.g., a concrete, a mortar), or a combination thereof. In general embodiments, a masonry coating may be selected to confer resistance to water (e.g., a salt water), resistance to acid conditions, alteration of appearance (e.g., color, brightness), or a combination thereof. Typically, a masonry coating comprises a multicoat system. In specific embodiments, a masonry multicoat system comprises a primer, a topcoat, or a combination thereof. Examples of a masonry primer include a rubber primer (e.g., a styrene-butadiene copolymer primer). In certain embodiments, a topcoat comprises a water-borne coating and/or a solvent borne coating. Examples of a water-borne coating that may be selected for a masonry topcoat include a latex coating, a water reducible polyvinyl acetate-coating, or a combination thereof. In certain aspects, a solvent-borne topcoat comprises a thermoplastic coating, a thermosetting coating, or a combination thereof. Examples of a thermosetting coating include an oil, an alkyd, a urethane, an epoxy, or a combination thereof. In certain aspects, a thermosetting coating comprises a multi-pack coating, such as, for example, an epoxy, a urethane, or a combination thereof. In specific aspects, a thermosetting coating undergoes film formation at ambient conditions. In other aspects, a thermosetting coating undergoes film formation at an elevated temperature such as a baking alkyd, a baking acrylic, a baking urethane, or a combination thereof. Examples of a thermoplastic coating include an acrylic, cellulosic, a rubber-derivative, a vinyl, or a combination thereof. In specific aspects, a thermoplastic coating comprises a lacquer.

A masonry surface basic in pH, such as, for example, a cement-based material and/or a calcareous stone (e.g., marble, limestone) may be damaging to certain coating(s). Specific procedures for determining the pH of a masonry surface have been described, for example, in “ASTM Book of Standards, Volume 06.02, Paint—Products and Applications; Protective Coatings; Pipeline Coatings,” D4262, 2002. Due to porosity and/or contact with an external environment, a masonry surface often accumulates dirt and other loose surface contaminants, which typically are removed prior to application of a coating. Specific procedures for preparative cleaning (e.g., abrading, acid etching) of a masonry surface (e.g., sandstone, clay brick, concrete) have been described, for example, in “ASTM Book of Standards, Volume 06.02, Paint—Products and Applications; Protective Coatings; Pipeline Coatings,” D4259-88, D4260-88D, 5107-90, D5703-95, D4261-83, and D4258-83, 2002. In certain embodiments, moisture at and/or near a masonry surface may be less suitable during application of a coating (e.g., a solvent-borne coating). Specific procedures for determining the presence of such moisture upon a masonry surface have been described, for example, in “ASTM Book of Standards, Volume 06.02, Paint—Products and Applications; Protective Coatings; Pipeline Coatings,” D4263-83, 2002. Specific procedures for determining the suitability of a coating and/or a film, particularly in conferring water resistance to a masonry surface, have been described, for example, in “ASTM Book of Standards, Volume 06.02, Paint—Products and Applications; Protective Coatings; Pipeline Coatings,” D6237-98, D4787-93, D5860-95, D6489-99, D6490-99, and D6532-00, 2002. Additional procedures for determining the suitability of a coating and/or a film for use as a masonry coating have been described, for example, in “Paint and Coating Testing Manual, Fourteenth Edition of the Gardner-Sward Handbook,” (Koleske, J. V. Ed.), pp. 725-730, 1995.

iii. Artist's Coatings

Artist coatings refer to a coating used by artists for a decorative purpose. Often, an artist's coating (e.g., paint) may be selected for durability for decades and/or centuries at ambient conditions, usually indoors. A coating such as an alkyd coating, an oil coating, an oleoresinous coating, an emulsion (e.g., acrylic emulsion) coating, or a combination thereof, are typically selected for use as an artist's coating.

Specific standards for physical properties, chemical properties, and/or procedures for determining the suitability (e.g., lightfastness) of a coating and/or a film for use as an artist's coating have been described, for example, in “ASTM Book of Standards, Volume 06.02, Paint—Products and Applications; Protective Coatings; Pipeline Coatings,” D4236-94, D5724-99, D4302-99, D4303-99, D4941-89, D5067-99, D5098-99, D5383-02, D5398-97, D5517-00, and D6801-02a, 2002; and in “Paint and Coating Testing Manual, Fourteenth Edition of the Gardner-Sward Handbook,” (Koleske, J. V. Ed.), pp. 706-710, 1995.

b. Industrial Coatings

An industrial coating comprises a coating applied to a surface of a manufactured product in a factory setting. An industrial coating typically undergoes film formation to produce a film with a protective and/or an aesthetic purpose. An industrial coating shares some similarities to an architectural coating, such as comprising similar coating components, being applied to the same material types of surfaces, being applied to an interior surface, being applied to an exterior surface, or a combination thereof. Examples of coating types that are commonly used for an industrial coating include an epoxy-coating, a urethane-coating, alkyd-coating, a vinyl-coating, chlorinated rubber-coating, or a combination thereof. Examples of a surface commonly coated by an industrial coating include a metal (e.g., an aluminum, a zinc, a copper, an alloy, etc); a glass; a plastic; a cement; a wood; a paper; or a combination thereof. An industrial coating may be storage stable for about 12 months or more, applied at ambient conditions, applied using a hand-held applicator, undergo film formation at ambient conditions, or a combination thereof.

However, an industrial coating often does not meet one or more of these characteristics previously described for an architectural coating. For example, an industrial coating may have a storage stability of days, weeks, or months, as due to a more rapid use rate in coating a factory prepared item. An industrial coating may be applied and/or undergo film formation at baking conditions. An industrial coating may be applied using techniques such as, for example, spraying by a robot, anodizing, electroplating, and/or laminating of a coating and/or a film onto a surface. In some embodiments, an industrial coating undergoes film formation by irradiating the coating with non-visible light electromagnetic radiation and/or particle radiation such as UV radiation, infrared radiation, electron-beam radiation, or a combination thereof.

In certain embodiments, an industrial coating comprises an industrial maintenance coating, which produces a protective film with excellent heat resistance (e.g., 121° C. or greater), solvent resistance (e.g., an industrial solvent, an industrial cleanser), water resistance (e.g., salt water, acidic water, alkali water), corrosion resistance, abrasion resistance (e.g., mechanical produced wear), or a combination thereof. An example of an industrial maintenance coating includes a high-temperature industrial maintenance coating, which may be applied to a surface intermittently and/or continuously contacted with a temperature of about 204° C. or greater. An additional example of an industrial maintenance coating comprises an industrial maintenance anti-graffiti coating, which comprises a two-pack clear coating applied to an exterior surface that may be intermittently contacted with a solvent and/or abrasion. Examples of coating types that are commonly used for an industrial maintenance coating include an epoxy-coating, a urethane-coating, an alkyd-coating, a vinyl-coating, a chlorinated rubber-coating, or a combination thereof.

Industrial coatings (e.g., coil coatings) and their use have been described in the art (see, for example, in “Paint and Surface Coatings: Theory and Practice,” 2^{nd} Edition, pp. 502-528, 1999; in “Paints, Coatings and Solvents,” 2^{nd} Edition, pp. 330-410, 1998; in “Organic Coatings: Science and Technology, Volume 1: Film Formation, Components, and Appearance,” 2^{nd} Edition, pp. 138, 317-318). Standard procedures for determining the properties of an industrial coating (e.g., an industrial wood coating, an industrial water-reducible coating) have been described, for example, in “ASTM Book of Standards, Volume 06.02, Paint—Products and Applications; Protective Coatings; Pipeline Coatings,” D4712-87a, D6577-00a, D2336-99, D3023-98, D3794-00, D4147-99, and D5795-95, 2002.

i. Automotive Coatings

An automotive coating refer to a coating used on an automotive vehicle, particularly those for civilian use. The manufacturers of a vehicle typically require that a coating conform to specific properties of weatherability (e.g., UV resistance) and/or appearance. Typically, an automotive coating comprises a multicoat system. In specific embodiments, an automotive multicoat system comprises a primer, a topcoat, or a combination thereof. Examples of an automotive primer include a nonweatherable primer, which lack sufficient UV resistance for single layer use, and/or a weatherable primer, which possesses sufficient UV resistance to be used without an additional layer. Examples of an automotive topcoat include an interior topcoat, an exterior topcoat, or a combination thereof.

Examples of a nonweatherable automotive primer include a primer applied by electrodeposition, a conductive (“electrostatic”) primer, and/or a nonconductive primer. In certain embodiments, a primer may be applied by electrodeposition, wherein a metal surface may be immersed in a primer, and electrical current promotes application of a primer component (e.g., a binder) to the surface. An example of a metal primer suitable for electrodeposition application includes a primer comprising an epoxy binder comprising an amino moiety, a blocked isocyanate urethane binder, and about 75% to about 95% aqueous liquid component. In other embodiments, a primer comprises a conductive primer, which allows additional coating layers to be applied using an electrostatic technique. A conductive primer may be applied to a plastic surface, including a flexible plastic surface and/or a nonflexible plastic surface. Such primers vary in their respective flexibility property to better suit use upon the surface. An example of a flexible plastic conductive primer includes a primer comprising a polyester binder, a melamine binder, and a conductive carbon black pigment. An example of a nonflexible plastic primer includes a primer comprising an epoxy ester binder and/or an alkyd binder, a melamine binder and conductive carbon black pigment. In certain embodiments, a melamine binder may be partly or fully replaced with an aromatic isocyanate urethane binder, wherein the coating comprises a two-pack coating. A nonconductive primer may be similar to a conductive primer, except the carbon-black pigment may be absent or reduced in content. In certain embodiments, a nonconductive primer comprises a metal primer, a plastic primer, or a combination thereof. In specific aspects, the nonconductive primer comprises a pigment for colorizing purposes.

Examples of a weatherable automotive primer include a primer/topcoat and/or a conductive primer. An example of a primer/topcoat includes a flexible plastic primer, with suitable weathering properties (e.g., UV resistance) to function as a single layer topcoat. Examples of a flexible plastic primer include a primer comprising an acrylic and/or polyester binder and a melamine binder. In certain embodiments, a melamine binder may be partly or fully replaced with an aliphatic isocyanate urethane binder, wherein the coating comprises a two-pack coating. A weatherable conductive primer may be similar to a weatherable primer/topcoat, including a conductive pigment. In specific aspects, a weatherable automotive primer comprises a pigment for colorizing purposes.

An interior automotive topcoat may be applied to a metal surface, a plastic surface, a wood surface, or a combination thereof. In certain aspects, an interior automotive topcoat comprises part of a multicoat system further comprising a primer. Examples of an interior automotive topcoat include a coating comprising a urethane binder, an acrylic binder, or a combination thereof.

An exterior automotive topcoat may be applied to a metal surface, a plastic surface, or a combination thereof. In certain aspects, an exterior automotive topcoat comprises part of a multicoat system further comprising a primer, a sealer, an undercoat, or a combination thereof. In certain embodiments, an exterior automotive topcoat comprises a binder capable of thermosetting in combination with a melamine binder. Examples of such a thermosetting binder include an acrylic binder, an alkyd binder, a urethane binder, a polyester binder, or a combination thereof. In certain embodiments, a melamine binder may be partly or fully replaced with a urethane binder, wherein the coating comprises a two-pack coating. In typical embodiments, an exterior automotive topcoat further comprises a light stabilizer, a UV absorber, or a combination thereof. In general aspects, an exterior automotive topcoat further comprises a pigment.

Specific procedures for determining the suitability of a coating (e.g., a nonconductive coating) and/or film for use as an automotive coating, including spray application suitability, coating VOC content and film properties (e.g., corrosion resistance, weathering) have been described, for example, in “ASTM Book of Standards, Volume 06.01, Paint—Tests for Chemical, Physical, and Optical Properties; Appearance,” D5087-02, D6266-00, and D6675-01, 2002; and “ASTM Book of Standards, Volume 06.02, Paint—Products and Applications; Protective Coatings; Pipeline Coatings,” D5066-91, D5009-02, D5162-01, and D6486-01, 2002; and in “Paint and Coating Testing Manual, Fourteenth Edition of the Gardner-Sward Handbook,” (Koleske, J. V. Ed.), pp. 711-716, 1995.

ii. Can Coatings

Can coatings refer to coatings used on a container (e.g., an aluminum container, a steel container), such as for a food, a chemical, or a combination thereof. The manufacturers of a can typically require that a coating conform to specific properties of corrosion resistance, inertness (e.g., to prevent flavor alterations in food, a chemical reaction with a container's contents, etc), appearance, durability, or a combination thereof. Typically, a can coating comprises an acrylic-coating, an alkyd-coating, an epoxy-coating, a phenolic-coating, a polyester-coating, a poly(vinyl chloride)-coating, or a combination thereof. Though a can may be made of the same or similar material, different surfaces of a can may require coating(s) of differing properties of inertness, durability and/or appearance. For example, a coating for a surface of the interior of a can that contacts the container's contents may be selected for a chemical inertness property, a coating for a surface at the end of a can may be selected for a physical durability property, or a coating for a surface on the exterior of a can may be selected for an aesthetic property. To meet the varying can's surface requirements, a can coating may comprise a multicoat system. In specific embodiments, a can multicoat system comprises a primer, a topcoat, or a combination thereof. In certain embodiments, an epoxy-coating, a poly(vinyl chloride-coating), or a combination thereof may be selected as a primer for a surface at the end of a can. In other embodiments, an oleoresinous-coating, a phenolic-coating, or a combination thereof may be selected as a primer for a surface in the interior of a can. In some aspects, a water-borne epoxy and acrylic-coating may be selected as a topcoat for a surface of an interior of a can. In additional embodiments, an acrylic-coating, an alkyd-coating, a polyester-coating, or a combination thereof may be selected as an exterior coating. In certain facets, a can coating (e.g., a primer, a topcoat) may comprise an amino resin, a phenolic resin, or a combination thereof for cross-linking in a thermosetting film formation reaction. In certain embodiments, a can coating may be applied to a surface by spray application. In other embodiments, a can coating undergoes film formation by UV irradiation. Specific procedures for determining the suitability of a coating and/or a film for use as a can coating, have been described, for example, in “Paint and Coating Testing Manual, Fourteenth Edition of the Gardner-Sward Handbook,” (Koleske, J. V. Ed.), pp. 717-724, 1995.

iii. Sealant Coatings

Sealant coatings refer to coatings used to fill a joint to reduce or prevent passage of a gas (e.g., air), water, a small material (e.g., dust), a temperature change, or a combination thereof. A sealant coating (“sealant”) may be thought of as a coating that bridges by contact two or more surfaces. A joint comprises a gap or opening between two or more surfaces, which may be of the same material type (e.g., a metal, a wood, a glass, a masonry, a plastic, etc). In typical embodiments, a joint has a width, a depth, a breadth, or a combination thereof, of about 0.64 mm to about 5.10 mm.

In certain embodiments, a sealant coating comprises an oil, a butyl, an acrylic, a blocked styrene, a polysulfide, a urethane, a silicone, or a combination thereof. A sealant may comprise a solvent-borne coating and/or a water-borne coating (e.g., a latex). In certain aspects, a sealant comprises a latex (e.g., an acrylic latex). In other embodiments, a sealant may be selected for flexibility, as one or more of the joint surfaces may move during normal use. Examples of a flexible sealant include a silicone, a butyl, an acrylic, a blocked styrene, an acrylic latex, or a combination thereof. An oil sealant typically comprises a drying oil, an extender pigment, a thixotrope, and a drier. A solvent-borne butyl sealant typically comprises a polyisobytylene and/or a polybutene, an extender pigment (e.g., talc, calcium carbonate), a liquid component, and an additive (e.g., an adhesion promoter, an antioxidant, a thixotrope). A solvent-borne acrylic sealant typically comprises a polymethylacrylate (e.g., a polyethyl, a polybutyl), a colorant, a thixotrope, an additive, and a liquid component. A solvent-borne blocked styrene sealant typically comprises a styrene, a styrene-butadiene, an isoprene, or a combination thereof, and a liquid component. A solvent-borne acrylic sealant, a blocked styrene sealant, or a combination thereof, may be selected for aspects wherein UV resistance may be desired. A urethane sealant may comprise an one-pack or two-pack coating. A solvent-borne one-pack urethane sealant typically comprises a urethane comprising a hydroxyl moiety, a filler, a thixotrope, an additive, an adhesion promoter, and a liquid component. A solvent-borne two-pack urethane sealant typically comprises a polyether comprising an isocyanate moiety in one-pack and a binder comprising a hydroxyl moiety in a second pack. A solvent-borne two-pack urethane sealant typically also comprises a filler, an adhesion promoter, an additive (e.g., a light stabilizer), or a combination thereof. In certain aspects, a solvent-borne urethane sealant may be selected for a sealant with a good abrasion resistance. A polysulfide sealant may comprise an one-pack or a two-pack coating. A solvent-borne one-pack polysulfide sealant typically comprises a urethane comprising a hydroxyl moiety, a filler, a thixotrope, an additive, an adhesion promoter, and a liquid component. A solvent-borne two-pack polysulfide sealant typically comprises a first pack, which typically comprises a polysulfide, an opacifing pigment, a colorizer (e.g., a pigment), a clay, a thixotrope (e.g., a mineral), and a liquid component; and a second pack, which typically comprises a curing agent (e.g., lead peroxide), an adhesion promoter, an extender pigment, and a light stabilizer. A silicone sealant typically comprises a polydimethyllsiloxane and a methyltriacetoxy silane, a methyltrimethoxysilane, a methyltricyclorhexylaminosilane, or a combination thereof. A water-borne acrylic latex sealant typically comprises a thermoplastic acrylic, a filler, a surfactant, a thixotrope, an additive, and a liquid component. Procedures for determining the suitability of a coating and/or a film for use as a sealant coating have been described, for example, in “Paint and Coating Testing Manual, Fourteenth Edition of the Gardner-Sward Handbook,” (Koleske, J. V. Ed.), pp. 735-740, 1995.

iv. Marine Coatings

A marine coating comprises a coating used on a surface that contacts water and/or a surface that comprises part of a structure continually near water (e.g., a ship, a dock, a drilling platform for fossil fuels, etc). Typically, such a surface comprises a metal, such as an aluminum, a high tensile steel, a mild steel, or a combination thereof. For embodiments wherein a surface contacts water, the type of marine coating may be selected to resist fouling, corrosion, or a combination thereof. Fouling refers to an accumulation of aquatic organisms, including microorganisms, upon a marine surface. Fouling may damage a film, and as many marine coatings are formulated with a preservative, an anti-corrosion property (e.g., an anticorrosion pigment), or a combination thereof, as such damage often leads to corrosion of metal surfaces. Additionally, a marine coating may be selected to resist fire, such as a coating applied to a surface of a ship. Further properties that are often used in a marine coating include chemical resistance, impact resistance, abrasion resistance, friction resistance, acoustic camouflage, electromagnetic camouflage, or a combination thereof.

To achieve the various properties of a marine coating, often a multicoat system may be used. For metal surfaces, a primer known as a blast primer may be applied to the surface within seconds of blast cleaning. Examples of a blast primer include a polyvinyl butyral (“PVB”) and phenolic resin coating; a two-pack epoxy coating; and/or a two-pack zinc and ethyl silicate coating. A marine metal surface undercoat and/or a topcoat typically comprises an alkyd coating, a bitumen coating, a polyvinyl coating, or a combination thereof. Marine coatings and their use are known in the art (see, for example, in “Paint and Surface Coatings Theory and Practice,” 2^{nd} Edition, pp. 529-549, 1999; in “Paints, Coatings and Solvents,” 2^{nd} Edition, pp. 252-258, 1998; in “Organic Coatings: Science and Technology, Volume 1: Film Formation, Components, and Appearance,” 2^{nd} Edition, pp. 138, 317-318). Specific procedures for determining the purity/properties of a marine coating, an anti-fouling coating, and/or a coating component thereof (e.g., a cuprous oxide, a copper powder, an organotin) under marine conditions (e.g., submergence, water based erosion, seawater biofouling resistance, barnacle adhesion resistance) and/or a marine film have been described, for example, in “ASTM Book of Standards, Volume 06.02, Paint—Products and Applications; Protective Coatings; Pipeline Coatings,” D3623-78a, D4938-89, D4939-89, D5108-90, D5479-94, D6442-99, D6632-01, D4940-98, and D5618-94, 2002; and “ASTM Book of Standards, Volume 06.03, Paint—Pigments, Drying Oils, Polymers, Resins, Naval Stores, Cellulosic Esters, and Ink Vehicles,” D912-81 and D964-65, 2002.

c. Specification Coatings

A specification coating may be formulated by selection of coating components to fulfill a set of requirements prescribed by a consumer. Examples a specification finish coating include a military specified coating, a Federal agency (e.g., Department of Transportation) specified coating, a state specified coating, or a combination thereof. A specification coating such as a chemical agent resistant coatings (“CARC”), a camouflage coating, or a combination thereof may be selected in certain embodiments for incorporation of a biomolecular composition. A camouflage coating comprises a coating that may be formulated with a material (e.g., a pigment) that reduces the visible differences between the appearance of a coated surface from the surrounding environment. Often, a camouflage coating may be formulated to reduce the detection of a coated surface by a devise that measures nonvisible light (e.g., infrared radiation). Various sources of specification coating requirements are described in, for example, “Paint and Coating Testing Manual, Fourteenth Edition of the Gardner-Sward Handbook,” (Koleske, J. V. Ed.), pp. 891-893, 1995).

i. Pipeline Coatings

An example of a specification coating comprises a pipeline (e.g., a metal pipeline) coating, such as one used to convey a fossil fuel. A pipeline coating may possess corrosion resistance, and an example of a pipeline coating includes a coal tar-coating, a polyethylene-coating, an epoxy powder-coating, or a combination thereof. A coal tar-coating may comprise, for example, a coal tar mastic-coating, a coal tar epoxide-coating, a coal tar urethane-coating, a coal tar enamel-coating, or a combination thereof. A coal tar mastic-coating typically comprises an extender, a vicosifier, or a combination thereof. In general aspects, a coal tar mastic-coating layer may comprise about 127 mm to about 160 mm thick. In embodiments wherein improved water resistance may be desired, a coal tar epoxide-coating may be selected. In embodiments wherein rapid film formation may be desired (e.g., pipeline repair), a coal tar urethane-coating may be selected. In embodiments wherein good water resistance, heat resistance up to about 82° C., bacterial resistance, poor UV resistance, or a combination thereof, may be suitable, a coal tar enamel may be selected. In embodiments wherein cathodic protection, physical durability, or a combination thereof may be desired, an epoxide powder-coating may be selected. In certain embodiments, an electrostatic spray applicator may be used to apply the powder coating. In certain embodiments, a pipeline coating comprises a multicoat system. In specific aspects, a pipeline multicoat system comprises an epoxy powder primer, a two-pack epoxy primer, a chlorinated rubber primer, or a combination thereof, and a polyethylene topcoat. Specific procedures for determining the suitability of a coating and/or a film for use as a pipeline coating, including coating storage stability (e.g., settling) and film properties (e.g., abrasion resistance, water resistance, flexibility, weathering, film thickness, impact resistance, chemical resistance, cathodic disbonding resistance, heat resistance) have been described, for example, in “ASTM Book of Standards, Volume 06.02, Paint—Products and Applications; Protective Coatings; Pipeline Coatings,” G6-88, G9-87, G10-83, G11-88, G12-83, G13-89, G20-88, G70-81, G8-96, G17-88, G18-88, G19-88, G42-96, G55-88, G62-87, G80-88, G95-87, and D6676-01e1, 2002; and in “Paint and Coating Testing Manual, Fourteenth Edition of the Gardner-Sward Handbook,” (Koleske, J. V. Ed.), pp. 731-734, 1995.

ii. Traffic Marker Coatings

A traffic marker coating comprises a coating (e.g., a paint) used to visibly convey information on a surface usually subjected to weathering and abrasion (e.g., a pavement). A traffic marker coating may comprise a solvent-borne coating and/or a water-borne coating. Examples of a solvent-borne traffic marker coating include an alkyd, a chlorinated rubber, or a combination thereof. In certain aspects, a solvent-borne coating may be applied by spray application. In some embodiments, a traffic marker coating comprises a two-pack coating, such as, for example, an epoxy-coating, a polyester-coating, or a combination thereof. In other embodiments, a traffic marker coating comprises a thermoplastic coating, a thermosetting coating, or a combination thereof. Examples of a combination thermoplastic/thermosetting coating include a solvent-borne alkyd and/or solvent-borne chlorinated rubber-coating. Examples of a thermoplastic coating include a maleic-modified glycerol ester-coating, a hydrocarbon-coating, or a combination thereof. In certain aspects, the thermoplastic coating comprises a liquid component, wherein the liquid component comprises a plasticizer, a pigment, and an additive (e.g., a glass bead).

Specific procedures for determining the suitability of a coating and/or a film for use as a traffic marker paint, including coating storage stability (e.g., settling), glass bead properties (e.g., reflectance), film durability (e.g., adhesion, pigment retention, solvent resistance, fuel resistance) and/or relevant film visual properties (e.g., retroreflectance, fluorescence) have been described, for example, in “ASTM Book of Standards, Volume 06.02, Paint—Products and Applications; Protective Coatings; Pipeline Coatings,” D713-90, D868-85, D969-85, D1309-93, D2205-85, D2743-68, D2792-69, D4796-88, D4797-88, D1155-89, D1214-89, and D4960-89, 2002; in “ASTM Book of Standards, Volume 06.01, Paint—Tests for Chemical, Physical, and Optical Properties; Appearance,” F923-00, E1501-99e1, E1696-02, E1709-00e1, E1710-97, E1743-96, E2176-01, E808-01, E809-02, E810-01, E811-95, D4061-94, E2177-01, E991-98, and E1247-92, 2002; and in “Paint and Coating Testing Manual, Fourteenth Edition of the Gardner-Sward Handbook,” (Koleske, J. V. Ed.), pp. 741-747, 1995.

iii. Aircraft Coatings

An aircraft coating protects and/or decorates a surface (e.g., metal, plastic) of an aircraft. Typically, an aircraft coating may be selected for excellent weathering properties, excellent heat and cold resistance (e.g., about −54° C. to about 177° C.), or a combination thereof. Specific procedures for determining the suitability of a coating and/or a film for use as aircraft coating, are described in, for example, in “Paint and Coating Testing Manual, Fourteenth Edition of the Gardner-Sward Handbook,” (Koleske, J. V. Ed.), pp. 683-695, 1995.

iv. Nuclear Power Plant Coatings

An additional example of a specification coating comprises a coating for a nuclear power plant, which generally possesses particular properties (e.g., gamma radiation resistance, chemical resistance), as described in “ASTM Book of Standards, Volume 06.02, Paint—Products and Applications; Protective Coatings; Pipeline Coatings,” D5962-96, D5163-91, D5139-90, D5144-00, D4286-90, D3843-00, D3911-95, D3912-95, D4082-02, D4537-91, D5498-01, and D4538-95, 2002.

### O. COATING COMPONENTS

In addition to the disclosures herein, the preparation and/or chemical synthesis of coating components, other than the biomolecular compositions described herein, have been described [see, for example, “Paint and Coating Testing Manual, Fourteenth Edition of the Gardner-Sward Handbook,” (Koleske, J. V., Ed.) (1995); “Paint and Surface Coatings: Theory and Practice, Second Edition,” (Lambourne, R. and Strivens, T. A., Eds.) (1999); Wicks, Jr., Z. W., Jones, F. N., Pappas, S. P. “Organic Coatings, Science and Technology, Volume 1: Film Formation, Components, and Appearance,” (1992); Wicks, Jr., Z. W., Jones, F. N., Pappas, S. P. “Organic Coatings, Science and Technology, Volume 2: Applications, Properties and Performance,” (1992); “Paints, Coatings and Solvents, Second, Completely Revised Edition,” (Stoye, D. and Freitag, W., Eds.) (1998); “Handbook of Coatings Additives,” 1987; In “Waterborne Coatings and Additives” 1995; “ASTM Book of Standards, Volume 06.01, Paint—Tests for Chemical, Physical, and Optical Properties; Appearance,” (2002); “ASTM Book of Standards, Volume 06.02, Paint—Products and Applications; Protective Coatings; Pipeline Coatings,” (2002); “ASTM Book of Standards, Volume 06.03, Paint—Pigments, Drying Oils, Polymers, Resins, Naval Stores, Cellulosic Esters, and Ink Vehicles,” (2002); and “ASTM Book of Standards, Volume 06.04, Paint—Solvents; Aromatic Hydrocarbons,” (2002)].

However, coating components are typically obtained from commercial vendors, which is a method of obtaining a coating component commonly used due to ease and reduced cost. Various texts, for example, Flick, E. W. “Handbook of Paint Raw Materials, Second Edition,” 1989, describes over 4,000 coating components (e.g., an antifoamer, an antiskinning agent, a bactericide, a binder, a defoamer, a dispersant, a drier, an extender, a filler, a flame/fire retardant, a flatting agent, a fungicide, a latex emulsion, an oil, a pigment, a preservative, a resin, a rheological/viscosity control agent, a silicone additive, a surfactant, a titanium dioxide, etc) provided by commercial vendors; and Ash, M. and Ash, I. “Handbook of Paint and Coating Raw Materials, Second Edition,” 1996, which describes over 18,000 coating components (e.g., an accelerator, an adhesion promoter, an antioxidant, an antiskinning agent, a binder, a coalescing agent, a defoamer, a diluent, a dispersant, a drier, an emulsifier, a fire retardant, a flow control agent, a gloss aid, a leveling agent, a marproofing agent, a pigment, a slip agent, a thickener, a UV stabilizer, viscosity control agent, a wetting agent, etc) provided by commercial vendors.

Specific commercial vendors are referred to herein as examples, and include Acima™ AG, Im Ochsensand, CH-9470 Buchs/SG; Air Products and Chemicals, Inc., 7201 Hamilton Boulevard, Allentown, Pa. 18195-1501; Arch Chemicals, Inc., 350Knotter Drive, Cheshire, Conn., 06410 U.S.A.; Avecia Inc., 1405 Foulk Road, PO Box 15457, Wilmington, Del. 19850-5457, U.S.A.; Bayer Corporation, 100 Bayer Rd., Pittsburgh, Pa. 15205-9741, U.S.A.; Buckman Laboratories, Inc., 1256 North McLean Blvd., Memphis, Tenn. 38108-0305, U.S.A.; BASF Corp., 100 Campus Drive, Florham Park, N.J. 07932; BYK-Chemie GmbH, Abelstrasse 45, P.O. Box 100245, D-46462 Wesel, Germany; Ciba Specialty Chemicals, 540 White Plains Road, P.O. Box 2005, Tarrytown, N.Y. 10591-9005, U.S.A.; Clariant LSM (America) Inc., 200 Rodney Building, 3411 Silverside Road, Wilmington, Del. 19810 U.S.A.; Cognis Corporation, 5051 Estecreek Drive, Cincinnati, Ohio 45232-1446, U.S.A.; Condea Servo LLC., 4081 B Hadley Road, South Plainfield, N.J. 07080-1114, U.S.A.; Cray Valley Limited, Waterloo Works, Machen, Caerphilly CF83 8YN United Kingdom; Dexter Chemical L.L.C. 845 Edgewater Road, Bronx, N.Y. 10474, USA; Dow Chemical Company, 2030 Dow Center, Midland, Mich. 48674 U.S.A.; Elementis Specialties, Inc., PO Box 700, 329 Wyckoffs Mill Road, Hightstown, N.J. 08520 U.S.A.; Goldschmidt Chemical Corp., 914 East Randolph Road PO Box 1299 Hopewell, Va. 23860 U.S.A.; Hercules Incorporated, 1313 North Market Street, Wilmington, Del. 19894-0001, U.S.A.; International Specialty Products, 1361 Alps Road, Wayne, N.J. 07470, U.S.A.; Octel-Starreon LLC USA, North American Headquarters, 8375 South Willow Street, Littleton, Colo. 80124, U.S.A.; Rohm and Haas Company, 100 Independence Mall West, Philadelphia, Pa. 19106-2399, U.S.A.; Solvay Advanced Functional Minerals, Via Varesina 2-4, 1-21021 Angera (VA); Troy Corporation, 8 Vreeland Road, PO Box 955, Florham Park, N.J., 07932 U.S.A.; R. T. Vanderbilt Company, Inc., 30 Winfield Street, Norwalk, Conn. 06855, U.S.A; Union Carbide Chemicals and Plastics Co., Inc., 39 Old Ridgebury Road, Danbury, Conn. 06817-0001, U.S.A.

1. Binders

A binder (“polymer,” “resin,” “film former”) comprises a molecule capable of film formation. Film formation refers to a physical and/or a chemical change of a binder in a coating, wherein the change converts the coating into a film. Often, a binder converts into a film through a polymerization reaction, wherein a first binder molecule covalently bonds with at least a second binder molecule to form a larger molecule, known as a “polymer.” As this process may be repeated a plurality of times, the composition converts from a coating comprising a binder into a film comprising a polymer.

A binder may comprise a monomer, an oligomer, a polymer, or a combination thereof. A monomer comprises a single unit of a chemical species that may undergo a polymerization reaction. However, a binder itself may comprise a polymer, as such larger binder molecules are more suitable for formulation into a coating capable of both being easily applied to a surface and undergoing an additional polymerization reaction to produce a film. An oligomer for use in a coating typically comprises about 2 to about 25 polymerized monomers.

A homopolymer comprises a polymer comprising monomers of the same chemical species. A copolymer comprises a polymer comprising monomers of at least two different chemical species. A linear polymer comprises an unbranched chain of monomers. A branched polymer comprises a branched (“forked”) chain of monomers. A network (“cross-linked”) polymer comprises a branched polymer wherein at least one branch forms an interconnecting covalent bond with at least one additional polymer molecule.

A thermoplastic binder and/or a coating reversibly softens and/or liquefies when heated. Film formation for a thermoplastic coating generally comprises a physical process, typically the loss of the volatile (e.g., liquid) component from a coating. As a volatile component may be removed, a solid film may be produced through entanglement of the binder molecules. In many aspects, a thermoplastic binder may comprise a higher molecular mass than a comparable thermosetting binder. In many aspects, a coating produced thermoplastic film may be susceptible to damage by a volatile component that may be absorbed by the film, which may soften and/or physically expand the film. In certain facets, a coating produced thermoplastic film may be removed from a surface by use of a volatile component. However, in many aspects, damage to a coating produced thermoplastic film may be repaired by application of a thermoplastic coating into the damaged areas and subsequent film formation.

A thermosetting binder undergoes film formation by a chemical process, typically the cross-linking of a binder into a network polymer. In certain embodiments, a thermosetting binder does not possess significant thermoplastic properties.

The glass transition temperature (“T_{g}”) refers to the temperature wherein the rate of increase of the volume of a binder and/or a film changes. Binders and films often do not convert from solid to liquid (“melt”) at a specific temperature (“Tₘ”), but rather possess a specific T_{g} wherein there is an increase in the rate of volume expansion with increasing temperature. At temperatures above the T_{g}, a binder and/or film becomes increasingly rubbery in texture until it becomes a viscous liquid. In certain embodiments described herein, a binder, particularly a thermoplastic binder, may be selected by its T_{g}, which provides guidance to the temperature range of film formation, as well as thermal and/or heat resistance of a film. The lower the T_{g}, the “softer” the resin, and generally, the film produced from such a resin. A softer film typically possesses greater flexibility (e.g., crack resistance) and/or a poorer resistance to dirt accumulation than a harder film.

In certain embodiments, a coating comprises a low molecular weight polymer, a high molecular weight polymer, or a combination thereof. Examples of a low molecular weight polymer include an alkyd, an amino resin, a chlorinated rubber, an epoxide resin, an oleoresinous binder, a phenolic resin, a urethane, a polyester, a urethane oil, or a combination thereof. Examples of a high molecular weight polymer include a latex, a nitrocellulose, a non-aqueous dispersion polymer (“NAS”), a solution acrylic, a solution vinyl, or a combination thereof. Examples of a latex include an acrylic, a polyvinyl acetate (“PVA”), a styrene/butadiene, or a combination thereof.

In addition to the disclosures herein, a binder, methods of binder preparation, commercial vendors of binder, and techniques in the art for using a binder in a coating may be used (see, for example, Flick, E. W. “Handbook of Paint Raw Materials, Second Edition,” pp. 287-805 and 879-998, 1989; in “Paint and Coating Testing Manual, Fourteenth Edition of the Gardner-Sward Handbook,” (Koleske, J. V. Ed.), pp. 23-29, 39-67, 74-84, 87, 268-285, 410, 539-540, 732, 735-736, 741, 770, 806-807, 845-849, and 859-861, 1995; in “Paint and Surface Coatings, Theory and Practice, Second Edition,” (Lambourne, R. and Strivens, T. A., Eds.), pp. 2-3, 7-10, 21, 24-40, 40-54, 60-71, 76, 81-86, 352, 358, 381-394, 396, 398, 405, 433-448, 494-497, 500, 537-540, 700-702, and 734, 1999; Wicks, Jr., Z. W., Jones, F. N., Pappas, S. P. “Organic Coatings, Science and Technology, Volume 1: Film Formation, Components, and Appearance,” pp. 39, 49-57, 62, 65-67, 67, 76-80, 83, 91, 104-118, 155, 168, 178, 182-183, 200, 202-203, 209, 214-216, 220 and 250, 162-186, 215-216 and 232, 59-60, 183-184, 133-143, 39, 144-161, 203, 219-220 and 239, 23, 110, 120-132, 122-130, 198, 202-203, 209 and 220, 60-62, 83-103, 164-167, 173, 177-178, 184-187, 195, 206, and 216-219, 1992; Wicks, Jr., Z. W., Jones, F. N., Pappas, S. P. “Organic Coatings, Science and Technology, Volume 2: Applications, Properties and Performance,” pp. 13-14, 18-19, 26, 33-34, 36, 41, 57, 77, 92, 95, 116-119, 143-145, 156, 161-165, 179-180, 191-193, 197-203, 210-211, 213-214, 216, 219-222, 230-239, 260-263, 269-271, 276-284, 288-293, 301-307, 310, 315-316, 319-321, and 325-346, 1992; and in “Paints, Coatings and Solvents, Second, Completely Revised Edition,” (Stoye, D. and Freitag, W., Eds.) pp. 5, 11-22, 37-50, 54-55, 72, 80-87, 96-98, 108, 126, and 136, 1998.

a. Oil-Based Binders

Certain binders, such as, for example, an oil (e.g., a drying oil), an alkyd, an oleoresinous binder, a fatty acid epoxide ester, or a combination thereof, are prepared and/or synthesized from an oil and/or a fatty acid, and undergo film formation by thermosetting oxidative cross-linking of fatty acids, and may be referred to herein as an “oil-based binder.” These types of binders often possess similar properties (e.g., solubility, viscosity). An oil-based binder coating often further comprises a drier, an antiskinning agent, an alkylphenolic resin, a pigment, an extender, a liquid component (e.g., a solvent), or a combination thereof. A drier, such as a primary drier, secondary drier, or a combination thereof, may be selected to promote film formation. In certain facets, an oil-based binder coating may comprise an anti-skinning agent, which may be used to control film-formation caused by a primary drier and/or oxidation. A liquid component may be selected, for example, to alter a rheological property (e.g., flow), wetting and/or dispersion, of a particulate material. In certain embodiments, a liquid component comprises a hydrocarbon. In particular embodiments, the hydrocarbon comprises an aliphatic hydrocarbon, an aromatic hydrocarbon (e.g., toluene, xylene), or a combination thereof. In some facets, the liquid component comprises, by weight, about 5% to about 20% of an oil-based binder coating.

In alternative embodiments, an oil-based temporary coating (e.g., a non-film forming coating) may be produced, for example, by inclusion of an antioxidant, reduction of the amount of a drier, selection of an oil-based binder comprising fewer or no double bonds, or a combination thereof.

An oil-based binder coating may be selected for embodiments wherein a relatively low viscosity may be desired, such as, for example, application to a corroded metal surface, a porous surface (e.g., wood), or a combination thereof, due to the penetration power of a low viscosity coating. In certain facets, application of an oil-binder coating produces a layer having less than about 25 μm on vertical surfaces and about 40 μm on horizontal surfaces to reduce shrinkage and/or wrinkling. Additionally, in aspects wherein the profile of the wood surface may be retained, such a thin film thickness may be used. In specific aspects, an oil-binder coating may be selected as a wood stain, a topcoat, or a combination thereof. In particular facets, a wood stain comprises an oil (e.g., linseed oil) coating, an alkyd, or a combination thereof. Often, wood coating comprises a lightstabilizer (e.g., UV absorber).

i. Oils

An oil comprises a polyol esterified to at least one fatty acid. A polyol (“polyalcohol,” “polyhydric alcohol”) comprises an alcohol comprising more than one hydroxyl moiety per molecule. In certain embodiments, an oil comprises an acylglycerol esterified to one fatty acid (“monacylglycerol”), two fatty acids (“diacylglycerol”), or three fatty acids (“triacylglycerol,” “triglyceride”). Typically, however, an oil may comprise a triacylglycerol. A fatty acid comprises an organic compound comprising a hydrocarbon chain that includes a terminal carboxyl moiety. A fatty acid may be unsaturated, monounsaturated, and polyunsaturated referring to whether the hydrocarbon chain possess no carbon double bonds, one carbon double bond, or a plurality of carbon double bonds (e.g., 2, 3, 4, 5, 6, 7, or 8 double bonds), respectively.

In typical use in a coating, a plurality of fatty acids forms covalent cross-linking bonds to produce a film in coatings comprising oil binders and/or other binders comprising a fatty acid. Usually oxidation through contact with atmospheric oxygen may be used to promote film formation. Exposure to light also enhances film formation. The ability of an oil to undergo film formation by chemical cross-linking relates to the content of chemically reactive double bonds available in the oil's fatty acids. Oils are generally a mixture of chemical species, comprising different combinations of fatty acids esterified to glycerol. The overall types and percentages of particular fatty acids that are comprised in oils affect the ability of the oil to be used as a binder. Oils may be classified as a drying oil, a semi-drying oil, or a non-drying oil depending upon the ability of the oil to cross-link into a dry film without additives (e.g., driers) at ambient conditions and atmospheric oxygen. A drying oil forms a dry film to touch upon cross-linking, a semi-drying oil forms a sticky (“tacky”) film to touch upon cross-linking, while a non-drying oil does not produce a tacky and/or a dry film upon cross-linking. In certain facets, film-formation of a non-chemically modified oil-binder coating may typically take from about 12 hours to about 24 hours, at ambient conditions, air, and lighting. Procedures for selection and testing of drying oils for a coating are described in, for example, “ASTM Book of Standards, Volume 06.03, Paint—Pigments, Drying Oils, Polymers, Resins, Naval Stores, Cellulosic Esters, and Ink Vehicles,” D555-84, 2002.

Drying oils comprise at least one polyunsaturated fatty acid to promote cross-linking. Polyunsaturated fatty acids (“polyenoic fatty acids”) include, but are not limited to, a 7,10,13-hexadecatrienoic (“16:3 n-3”); a linoleic [“9,12-octadecadienoic,” “18:2(n-6)”]; a γ-linolenic [“6,9,12-octadecatrienoic,” “18:3(n-6)”]; a trienoic 20:3(n-9); a dihomo-γ-linolenic [“8,11,14-eicosatrienoic,” “20:3(n-6)”]; an arachidonic [“5,8,11,14-eicosatetraenoic,” “20:4(n-6)”]; a licanic, (“4-oxo 9c11t13t-18:3”); a 7,10,13,16-docosatetraenoic [“22:4(n-6)”]; a 4,7,10,13,16-docosapentaenoic [“22:5(n-6)”]; a α-linolenic [“9,12,15-octadecatrienoic,” “18:3(n-3)”]; a stearidonic [“6,9,12,15-octadecatetraenoic,” “18:4(n-3)”]; a 8,11,14,17-eicosatetraenoic [“20:4(n-3)”]; a 5,8,11,14,17-eicosapentaenoic [“EPA,” “20:5(n-3)”]; a 7,10,13,16,19-docosapentaenoic [“DPA,” “22:5(n-3)”]; a 4,7,10,13,16,19-docosahexaenoic [“DHA,” “22:6(n-3)”]; 5,8,11-eicosatrienoic [“Mead acid,” “20:3(n-9)”]; a taxoleic (“all-cis-5,9-18:2”); a pinolenic (“all-cis-5,9,12-18:3”); a sciadonic (“all-cis-5,11,14-20:3”); a dihomotaxoleic (“7,11-20:2”); a cis-9, cis-15 octadecadienoic (“9,15-18:2”); a retinoic; or a combination thereof.

Drying oils may be further characterized as non-conjugated or conjugated drying oils depending upon whether their abundant fatty acid comprises a polymethylene-interrupted double bond or a conjugated double bond, respectively. A polymethylene-interrupted double bond comprises two double bonds separated by two or more methylene moieties. A polymethylene-interrupted fatty acid comprises a fatty acid comprising such a configuration of double bonds. Examples of polymethylene-interrupted fatty acids include a taxoleic, a pinolenic, a sciadonic, a dihomotaxoleic, a cis-9, cis-15 octadecadienoic, a retinoic, or a combination thereof.

A conjugated double bond comprises a moiety wherein a single methylene moiety connects a pair of carbon chain double bonds. A conjugated fatty acid comprises a fatty acid comprising such a pair of double bonds. A conjugated double bond may be more prone to cross-linking reactions than non-conjugated double bonds. A conjugated diene fatty acid, a conjugated triene fatty acid or a conjugated tetraene fatty acid, possesses two, three or four conjugated double bonds, respectively. An example of a common conjugated diene fatty acid comprises a conjugated linoleic. Examples of a conjugated triene fatty acid include an octadecatrienoic, a licanic, or a combination thereof. Examples of an octadecatrienoic acid include an α-eleostearic comprising the 9c,11t, 13t isomer, a calendic comprising a 8t, 10t, 12c isomer, a catalpic comprising the 9c, 11t, 13c isomer, or a combination thereof. An example of a conjugated tetraene fatty acid comprises a α-parinaric comprising the 9c, 11t, 13t, 15c isomer, and a 6-parinaric comprising the 9t, 11t, 13t, 15t isomer, or a combination thereof.

An oil for use in a coating may be obtained from renewable biological source, such as a plant, a fish, or a combination thereof. Examples of a plant oil commonly used in a coating and/or a coating component include a cottonseed oil, a linseed oil, an oiticica oil, a safflower oil, a soybean oil, a sunflower oil, a tall oil, a rosin, a tung oil, or a combination thereof. An example of a fish oil commonly used in a coating and/or a coating component includes a caster oil. A colder environment generally promotes a higher polyunsaturated fatty acid content in an organism (e.g., a sunflower). A cottonseed oil comprises about 36% saturated fatty acids, about 24% oleic, and about 40% linoleic. A castor oil comprises about 3% saturated fatty acids, about 7% oleic, about 3% linoleic, and about 87% ricinoleic (“12-hydroxy-9-octadecenoic”). A linseed oil comprises about 10% saturated fatty acids, about 20% to about 24% oleic (“cis-9-octadecenoic”), about 14% to about 19% linoleic, and about 48% to about 54% linolenic. An oiticica oil comprises about 16% saturated fatty acids, about 6% oleic, and about 78% licanic. A safflower oil comprises about 11% saturated fatty acids, about 13% oleic, about 75% linoleic, and about 1% linolenic. A soybean oil comprises about 14% to about 15% saturated fatty acids, about 22% to about 28% oleic, about 52% to about 55% linoleic, and about 5% to about 9% linolenic. A tall oil, which may comprise a product of paper production and may be in the form of a triglyceride, often comprises about 3% saturated fatty acids, about 30% to about 35% oleic, about 35% to about 40% linoleic, about 2% to about 5% linolenic, and about 10% to about 15% of a combination of pinolenic and conjugated linoleic. A rosin may comprise a combination of acidic compounds isolated during paper production, such as, for example, an abietic acid, a neoabietic acid, a dihydroabietic acid, a tetraabietic acid, an isodextropimaric acid, a dextropimaric acid, a dehydroabietic acid, and a levopimaric acid. A tung oil comprises about 5% saturated fatty acids, about 8% oleic, about 4% linoleic, about 3% linolenic, and about 80% α-elestearic. Standards for physical properties, chemical properties, and/or procedures for testing the purity/properties of various oils (e.g., a caster, a linseed, an oiticica, a safflower, a soybean, a sunflower, a tall, a tung, a rosin, a dehydrated caster, a boiled linseed, a drying oil, a fish oil, a heat-bodied drying oil) for use in a coating are described, for example in “ASTM Book of Standards, Volume 06.03, Paint—Pigments, Drying Oils, Polymers, Resins, Naval Stores, Cellulosic Esters, and Ink Vehicles,” D555-84, D960-02a, D961-86, D234-82, D601-87, D1392-92, D1462-92, D12-88, D1981-02, D5768-95, D3169-89, D260-86, D124-88, D803-02, D1541-97, D1358-86, D1950-86, D1951-86, D1952-86, D1954-86, D1958-86, D464-95, D465-01, D1959-97, D1960-86, D1962-85, D1964-85, D1965-87, D1966-69, D1967-86, D3725-78, D1466-86, D890-98, D1957-86, D1963-85, D5974-00, D1131-97, D1240-02, D889-99, D509-98, D269-97, D1065-96, and D804-02, 2002.

In certain embodiments, an oil comprises a chemically modified oil, which comprises an oil altered by a reaction thought to promote limited cross-linking. Generally, such a modified oil possesses an altered property, such as a higher viscosity, which may be more suitable for a particular coating application. Examples of a chemically modified oil include a bodied oil, a blown oil, a dimer acid, or a combination thereof. A bodied oil (“heat bodied oil,” “stand oil”) may be produced, for example, by heating a nonconjugated oil (e.g., about 320° C.) and/or a conjugated oil (e.g., about 240° C.) in a chemically unreactive atmosphere to promote limited cross-linking. A blown oil may be produced, for example, by passing air through a drying oil at, for example, about 150° C. A dimer acid may be produced, for example, by acid catalyzed dimerization and/or oligomerization of a polyunsaturated acid.

In certain embodiments, an oil comprises a synthetic conjugated oil, which comprises an oil altered by a reaction thought to produce a conjugated double bond in a fatty acid of the oil. A conjugated fatty acids have been produced from a nonconjugated fatty acid by alkaline hydroxide catalyzed reaction(s). However, a synthetic conjugated oil may comprise a semi-drying in air catalyzed film formation at ambient conditions, and a coating comprising such an oil may be cured by baking. Additionally a richinoleic acid, which may be obtained from a castor oil, may be dehydrogenated to produce a mixture of a conjugated and a non-conjugated fatty acid. A dehydrogenated castor oil comprises about 2% to about 4% saturated fatty acids, about 6% to about 8% oleic, about 48% to about 50% linoleic, and about 40% to about 42% conjugated linoleic.

Certain other compounds comprising a fatty acid and a polyol are classified herein as an oil for use as a binder such as a high ester oil, a maleated oil, or a combination thereof. A high ester oil comprises a polyol capable of comprising greater than three fatty acid esters per molecule and at least one fatty acid ester. However, a high ester oil may comprise four or more fatty acid esters per molecule. Examples of such a polyol include a pentaerythritiol, a dipentaerythritiol, a tripentaerythritiol, and/or a styrene/allyl alcohol copolymer. A high ester oil generally forms a film more rapidly than an acylglycerol based oil, as the opportunity for cross-linking reactions between fatty acids increases with the number of fatty acids attached to a single polyol. A maleated oil comprises an oil modified by a chemical reaction with a maleic anhydride. A maleic acid and an unsaturated and/or a polyunsaturated fatty acid react to produce a fatty acid with an additional acid moiety(s). A maleated oil may be more hydrophilic and/or has a faster film formation time than a comparative non-maleated oil.

ii. Alkyd Resins

In certain embodiments, a binder may comprise an alkyd resin. In general embodiments, an alkyd-coating may be selected as an architectural coating, a metal coating, a plastic coating, a wood coating, or a combination thereof. In certain aspects, an alkyd coating may be selected for use as a primer, an undercoat, a topcoat, or a combination thereof. In particular aspects, an alkyd coating comprises a pigment, an additive, or a combination thereof.

An alkyd resin comprises a polyester prepared from a polyol, a fatty acid, and a polybasic (“polyfunctional”) organic acid and/or an acid anhydride. An alkyd resin may be produced by first preparing monoacylpolyol, which comprises a polyol esterified to one fatty acid. The monoacylpolyol may be polymerized by an ester linkage(s) with a polybasic acid to produce an alkyd resin of desired viscosity in a solvent. Examples of a polyol include a 1,3-butylene glycol; a diethylene glycol; a dipentaerythritol; an ethylene glycol; a glycerol; a hexylene glycol; a methyl glucoside; a neopentyl glycol; a pentaerythritol; a pentanediol; a propylene glycol; a sorbitol; a triethylene glycol; a trimethylol ethane; a trimethylol propane; a trimethylpentanediol; or a combination thereof. In certain aspects, a polyol comprises an ethylene glycol; a glycerol; a neopentyl glycol; a pentaerythritol; a trimethylpentanediol; or a combination thereof. Examples of a polybasic acid andor an acid anhydride include an adipic acid, an azelaic acid, a chlorendic anhydride, a citric acid, a fumaric acid, an isophthalic acid, a maleic anhydride, a phthalic anhydride, a sebacic acid, a succinic acid, a trimelletic anhydride, or a combination thereof. In certain aspects, a polybasic acid and/or an acid anhydride comprises an isophthalic acid, a maleic anhydride, a phthalic anhydride, a trimelletic anhydride, or a combination thereof. Examples of a fatty acid include an abiatic, a benzoic, a caproic, a caprylic, a lauric, a linoleic, a linolenic, an oleic, a tertiary-butyl benzoic acid, a fatty acid from an oil/fat (e.g., a castor, a coconut, a cottonseed, a tall, a tallow), or a combination thereof. In certain aspects, a fatty acid comprises a benzoic, a fatty acid from tall oil, or a combination thereof. In specific aspects, an oil may be used in the reaction directly as a source of a fatty acid and/or a polyol. Examples of an oil include a castor oil, a coconut oil, a corn oil, a cottonseed oil, a dehydrated castor oil, a linseed oil, a safflower oil, a soybean oil, a tung oil, a walnut oil, a sunflower oil, a menhaden oil, a palm oil, or a combination thereof. In some aspects, an oil comprises a coconut oil, a linseed oil, a soybean oil, or a combination thereof.

In addition to the standards and analysis techniques previously described for an oil, standards for physical properties, chemical properties, and/or procedures for testing the purity/properties of various fatty acids (e.g., a fatty acid of a coconut, a corn, a cottonseed, a dehydrated caster, a linseed, a soybean, a tall oil, a rosin) and/or a polyol (e.g., a pentaerythritol, a hexylene glycol, an ethylene glycol, a diethylene glycol, a propylene glycol, a dipropylene glycol) and/or an acid anhydride (e.g., a phthalic anhydride, a maleic anhydride) for use in an alkyd and/or other coating component are described, for example, in “ASTM Book of Standards, Volume 06.03, Paint—Pigments, Drying Oils, Polymers, Resins, Naval Stores, Cellulosic Esters, and Ink Vehicles,” D1537-60, D1538-60, D1539-60, D1841-63, D1842-63, D1843-63, D5768-95, D1981-02, D1982-85, D1980-87, D804-02, D1957-86, D464-95, D465-01, D1963-85, D5974-00, D1466-86, D2800-92, D1585-96, D1467-89, and D1983-90, 2002; and in “ASTM Book of Standards, Volume 06.04, Paint—Solvents; Aromatic Hydrocarbons,” D2403-96, D3504-96, D2930-94, D3366-95, D3438-99, D2195-00, D2636-01, D2693-02, D2694-91, D5164-91, D1257-90, and D1258-95, 2002. Further, the composition, properties and/or purity of an alkyd resin and/or a solution comprising an alkyd resin selected for use in a coating such as a phthalic anhydride content, an isophthalic acid content, an unsaponifiable matter content, a fatty acid content/identification, a polyhydric alcohol content/identification, a glycerol, an ethylene glycol and/or a pentaerythirol content, and a silicon content may be empirically determined (see, for example, “ASTM Book of Standards, Volume 06.03, Paint—Pigments, Drying Oils, Polymers, Resins, Naval Stores, Cellulosic Esters, and Ink Vehicles,” D2689-88, D563-88, D2690-98, D2998-89, D1306-88, D1397-93, D1398-93, D2455-89, D1639-90, D1615-60, and D2456-91, 2002).

I. Oil Length Alkyd Binders

In specific embodiments, an alkyd resin may be selected based on the materials used in its preparation, which typically affect the alkyd's properties. In general aspects, an alkyd resin may be classified and/or selected for use in a particular application by its oil content, as the oil content affects the alkyd resin properties. Oil content refers to the amount of an oil relative to the solvent-free alkyd resin. Based on oil content, an alkyd resin may be classified as a very long oil alkyd resin, a long oil alkyd resin, a medium oil alkyd resin, or a short oil alkyd resin. Generally, the greater the oil content classification of an alkyd resin in a coating, the greater the ease of brush application, the slower the rate of film formation, the greater the film's flexibility, the poorer the chemical resistance of the film, the poorer the retention of gloss in an exterior environment, or a combination thereof. A short oil alkyd, a medium oil alkyd, a long oil alkyd, and a very long oil alkyd has an oil content range of about 1% to about 40%, about 40% to about 60%, about 60% to about 70%, and about 70% to about 85%, respectively, respectively. In typical embodiments, a short oil alkyd, a medium oil alkyd, a long oil alkyd, and a very long oil alkyd resin and/or such a coating comprise about 50%, about 45% to about 50%, about 60% to about 70%, or about 85% to about 100% nonvolatile component, respectively.

In certain embodiments, a short oil alkyd coating may be selected as an industrial coating. In certain aspects, a short oil alkyd may be synthesized from an oil, wherein the oil comprises a castor, a dehydrated castor, a coconut, a linseed, a soybean, a tall, or a combination thereof. In some aspects, the oil of a short oil alkyd comprises a saturated fatty acid. Examples of a saturated fatty acid include, but are not limited to, a caproic (“hexanoic,” “6:0”); a caprylic (“octanoic,” “8:0”); a lauric (“dodecanoic,” “12:0”); or a combination thereof. In particular facets, a short oil alkyd coating comprises a solvent, wherein the solvent comprises an aromatic hydrocarbon, an isobutanol, a VMP naphtha, a xylene, or a combination thereof. In other facets, the aromatic solvent comprises a high boiling aromatic solvent. In some aspects, a short oil alkyd may be insoluble or poorly soluble in an aliphatic hydrocarbon. In further embodiments, a short oil alkyd coating undergoes film formation by baking.

In certain embodiments, a medium oil alkyd coating may be selected as a farm implement coating, a railway equipment coating, a maintenance coating, or a combination thereof. In certain aspects, a medium oil alkyd may be synthesized from an oil, wherein the oil comprises a linseed, a safflower, a soybean, a sunflower, a tall, or a combination thereof. In some aspects, the oil of a medium oil alkyd comprises a monounsaturated fatty acid (e.g., an oleic acid). In particular facets, a medium oil alkyd coating comprises a solvent, wherein the solvent comprises an aliphatic hydrocarbon, an aromatic hydrocarbon, or a combination thereof.

In certain embodiments, a tall oil alkyd coating may be selected as an architectural coating, a maintenance coating, a primer, a topcoat, or a combination thereof. In certain aspects, a tall oil alkyd may be synthesized from an oil, wherein the oil comprises a linseed, a safflower, a soybean, a sunflower, a tall, or a combination thereof. In some aspects, the oil of a long oil alkyd comprises a polyunsaturated fatty acid. In particular facets, a tall oil alkyd coating comprises a solvent, wherein the solvent comprises an aliphatic hydrocarbon.

In certain embodiments, a very long oil alkyd coating may be selected as a latex architectural coating, a wood stain, or a combination thereof. In certain aspects, a very long oil alkyd may be synthesized from an oil, wherein the oil comprises a linseed, a soybean, a tall, or a combination thereof. In some aspects, the oil of a long oil alkyd comprises a polyunsaturated fatty acid. In particular facets, a very long oil alkyd coating comprises a solvent, wherein the solvent comprises an aliphatic hydrocarbon.

II. High Solid Alkyd Coatings

A high solid alkyd possesses a reduced viscosity, a lower average molecular weight, or a combination thereof. A high solid alkyd may be selected for embodiments wherein a reduced quantity liquid content (e.g., solvent) of a coating may be desired. In some embodiments, a high solid alkyd coating comprises an enamel coating. In other aspects, a high solid long and/or very long oil alkyd coating comprises an architectural coating. In further aspects, a high solid medium oil alkyd coating comprises a transportation coating. In further aspects, a high solid short oil alkyd coating comprises an industrial coating. Additional, various chemical moiety(s) may be incorporated in an alkyd to modify a property. Examples of such a moiety include an acrylic, a benzoic acid, an epoxide, an isocyanate, a phenolic, a polyamide, a rosin, a silicon, a styrene (e.g., a paramethyl styrene), a vinyl toluene, or a combination thereof. In certain embodiments, a benzoic acid modified high solid alkyd coating comprises a coating for a tool. In other embodiments, a phenolic modified high solid alkyd coating comprises a primer. A silicone modified alkyd coating may be selected for improved weather resistance, heat resistance, or a combination thereof. In specific aspects, a silicone modified alkyd coating may comprise an additional binder capable of cross-linking with the silicone moiety (e.g., a melamine formaldehyde resin). In specific facets, a silicone modified alkyd coating may be selected as a coil coating, an architectural coating, a metal coating, an exterior coating, or a combination thereof. In certain facets, a high solid silicon-modified alkyd coating may substitute an oxygenated compound (e.g., a ketone, an ester) for an aromatic hydrocarbon liquid component. However, a high solid silicon-modified alkyd coating, to achieve cross-linking during film-formation, may comprise an additional binder capable of cross-linking. In further embodiments, a silicone modified high solid alkyd coating comprises a maintenance coating, a topcoat, or a combination thereof.

III. Uralkyd Coatings

An uralkyd binder (“uralkyd,” “urethane alkyd,” “urethane oil,” “urethane modified alkyd”) comprises an alkyd binder, with the modification that compound comprising plurality of diisocyanate moieties partly or fully replacing the dibasic acid (e.g., a phthalic anhydride) in the synthesis reaction(s). Examples of an isocyanate comprising compounds include a 1,6-hexamethylene diisocyanate (“HDI”), a toluene diisocyanate (“TDI”), or a combination thereof. An uralkyd binder may be selected for embodiments wherein an improved abrasion resistance, improved resistance to hydrolysis, or a combination thereof, relative to an alkyd, may be desired in a film. However, an uralkyd binder prepared using TDI often has greater viscosity in a coating, reduced color retention in a film, or a combination thereof, relative to an alkyd binder. Additionally, an uralkyd binder prepared using an aliphatic isocyanate generally possesses improved color retention to an uralkyd prepared from TDI. An uralkyd coating tends to undergo film formation faster than a comparable alkyd binder, due to a generally greater number of available conjugated double bonds, an increased T_{g} in an uralkyd binder prepared using an aromatic isocyanate, or a combination thereof. A film comprising an uralkyd binder tends to develop a yellow to brown color. An uralkyd binder may be used in preparation of an architectural coating such as a varnish, an automotive refinish coating, or a combination thereof. Examples of a surface where an uralkyd coating may be applied include a furniture surface, a wood surface, and/or a floor surface.

IV. Water-Borne Alkyd Coatings

In general embodiments, an alkyd coating comprises a solvent-borne coating. However, an alkyd (e.g., a chemically modified alkyd) may be combined with a coupling solvent and water to produce a water-borne alkyd coating. Examples of a coupling solvent that may confer water reducibility to an alkyd resin includes an ethylene glucol monobutyether, a propylene glycol monoethylether, a propylene glycol monopropylether, an alcohol whose carbon content comprises four carbon atoms (e.g., s-butanol), or a combination thereof. In certain embodiments, a water-borne long oil alkyd coating may be selected as a stain, an enamel, or a combination thereof. In other embodiments, a water-borne medium oil alkyd coating may be selected as an enamel, an industrial coating, or a combination thereof. In further facets, a water-borne medium oil alkyd coating may undergo film formation by air oxidation. In other embodiments, a water-borne short oil alkyd coating may be selected as an enamel, an industrial coating, or a combination thereof. In further facets, a water-borne short oil alkyd coating may undergo film formation by baking.

iii. Oleoresinous Binders

An oleoresinous binder may be prepared from heating a resin and an oil. Examples of a resin typically used in the preparation of an oleoresinous binder include resins obtained from a biological source (e.g., a wood resin, a bitumen resin); a fossil source (e.g., a copal resin, a Kauri gum resin, a rosin resin, a shellac resin); a synthetic source (e.g., a rosin derivative resin, a phenolic resin, an epoxy resin); or a combination thereof. An example of an oil typically used in the preparation of an oleoresinous binder includes a vegetable oil, particularly an oil comprising a polyunsaturated fatty acid such as a tung, a linseed, or a combination thereof. The type of resin and oil used may identify an oleoresinous binder such as a copal-tung oleoresinous binder, a rosin-linseed oleoresinous binder, etc. An oleoresinous binder generally may be used in a clear varnish such as a lacquer, as well as in applications as a primer, an undercoat, a marine coating, or a combination thereof. In addition to the standards and analysis techniques previously described for an oil, standards for physical properties, chemical properties, and/or procedures for testing the purity/properties (e.g., T_{g}, molecular weight, color stability) of a hydrocarbon resin (e.g., a synthetic source resin) for use in an oleoresinous binder and/or other coating component are described, for example, in “ASTM Book of Standards, Volume 06.03, Paint—Pigments, Drying Oils, Polymers, Resins, Naval Stores, Cellulosic Esters, and Ink Vehicles,” E28-99, D6090-99, D6440-01, D6493-99, D6579-00, D6604-00, and D6605-00, 2002.

Similar to alkyd resins, oleoresinous binders may be categorized by oil length as a short oil or long oil oleoresinous binder, depending whether oil length comprises about 1% to about 67% or about 67% to about 99% oil, respectively. A short oil oleoresinous binder generally dries fast and/or form relatively harder, less flexible films, and are used, for example, for a floor varnish. A long oil oleoresinous binders generally dries slower and/or form a relatively more flexible film, and are used, for example, as an undercoat, an exterior varnish, or a combination thereof.

iv. Fatty Acid Epoxy Esters

In certain facets, an epoxy coating may be cured by fatty acid oxidation rather than an epoxide moiety and/or a hydroxyl moiety cross-linking reaction(s). A fatty acid epoxide ester resin comprises an ester of an epoxide resin and a fatty acid, which may be used to produce an ambient cure coating that undergoes film formation by an oxidative reaction as an oil-based coating. In certain embodiments, an epoxy resin may be selected with an epoxy equivalent weight of about 800 to about 1000. A short, a medium, and a long oil epoxide ester resin comprise about 30% to about 50%, about 50% to about 70%, or about 70% to about 90% fatty acid esterification, respectively, with similar, though sometimes improved, properties relative to an analogous alkyd. An epoxide ester resin produced film may be reduced in chemical resistance than a film produced by an epoxy and a curing agent comprising an amine. An epoxy ester resin may be selected as a substitute for an alkyd, a marine coating, an industrial maintenance coating, a floor topcoat, or a combination thereof.

b. Polyester Resins

A polyester resin (“polyester,” “oil-free alkyd”) comprises a polyester chemical, other than an alkyd resin, capable as use as a binder. A polyester resin may be chemically very similar to an alkyd, though the oil content may be about 0%. Consequently, a polyester-coating does not form cross-linking bonds by fatty acids oxidation during thermosetting film formation, but rather may be combined with an additional binder to form a cross-linked film. The selection of a polyester and an additional binder combination may be determined by the polyester's cross-linkable moiety(s). For example, a hydroxy-terminated polyester comprises a polyester produced by an esterification reaction comprising a molar excess of a polyol, and may be cross-linked with a urethane, an amino resin, or a combination thereof. A hydroxy-terminated polyester's hydroxyl moiety may react with a urethane's isocyanate moiety such as at ambient conditions and/or low-bake conditions, while such a polyester generally undergoes film formation at baking temperatures with an amino resin. In another example, a “carboxylic acid-terminated polyester” comprises a polyester produced by an esterification reaction comprising a molar excess of a polycarboxylic acid, and may be cross-linked with a urethane, an amino resin, a 2-hydroxylakylamide, or a combination thereof.

In general embodiments, a polyester-coating possesses improved color retention, flexibility, hardness, weathering, or a combination thereof, relative to an alkyd-coating. In some embodiments, a polyester resin may be selected to produce a coating for a metal surface. Generally, a polyester-coating possesses an improved adhesion property on a metal surface than a thermosetting acrylic-coating. Often, a polyester-coating comprises a thermosetting coating, particularly in embodiments for use upon a metal surface. However, a polyester-coating generally comprises an ester linkage that may be susceptible to hydrolysis, such as occurs in applications wherein such a polyester-coating contacts water.

A polyester resin may be prepared by an acid catalyzed esterification of a polyacid (e.g., a polycarboxylic acid, an aromatic polyacid) and a polyalcohol. A “polyacid” (“polybasic acid”) comprises a chemical comprising more than one acid moiety. Typically, a polyacid used in the preparation of a polyester comprise two acidic moieties, such as, for example, an aromatic dibasic acid, an anhydride of an aromatic dibasic acid, an aliphatic dibasic acid, or a combination thereof. Usually, a polyester resin comprises a plurality of polycarboxylic acids and/or polyalcohols, and such a polyester resin may be known herein as a “copolyester resin.” Examples of a polycarboxylic acid commonly used to prepare a polyester resin includes an adipic acid (“AA”); an azelic acid (“AZA”); a dimerized fatty acid; a dodecanoic acid; a hexahydrophthalic anhydride (“HHPA”); an isophthalic acid (“IPA”); a phthalic anhydride (“PA”); a sebacid acid; a terephthalic acid; a trimellitic anhydride; or a combination thereof. Examples of a polyalcohol commonly used to prepare a polyester resin include a 1,2-propanediol; a 1,4-butanediol; a 1,4-cyclohexanedimethanol (“CHDM”); a 1,6-hexanediol (“HD”); a diethylene glycol; an ethylene glycol; a glycerol; a neopentyl glycol (“NPG”); a pentaerythitol (“PE”); a trimethylolpropane (“TMP”); or a combination thereof. In certain embodiments, a polyester may be selected that has been synthesized by an acid catalyzed esterification reaction between a plurality of polyalcohols comprising two hydroxy moieties (a “diol”), a polyalcohol comprising three hydroxy moieties (a “triol”), and a dibasic acid. An example of a diol includes a 1,4-cyclohexanedimethanol; a 1,6-hexanediol; a neopentyl glycol; or a combination thereof. An example of a triol includes a trimethylolpropane. An example of a polyol comprising four hydroxy moieties (a “tetraol”) includes a pentaerythitol. In addition to the standards and analysis techniques previously described for an oil, an alkyd, a polyol, and/or an acid anhydride, standards for physical properties, chemical properties, and/or procedures for testing the purity/properties of a polyester are described, for example, in “ASTM Book of Standards, Volume 06.03, Paint—Pigments, Drying Oils, Polymers, Resins, Naval Stores, Cellulosic Esters, and Ink Vehicles,” D2690-98 and D3733-93, 2002.

The selection of a polyacid and/or a polyalcohol often affects a property of the polyester resin, such as the resistance of the polyester resin to hydrolysis, and similarly the water resistance of a coating and/or a film comprising such a polyester resin. In embodiments wherein a polyester-coating may be desired with an improved water resistance property relative to an other type of a polyester-coating, the coating may comprise a polyester prepared with a polyol that may be more difficult to esterify, and thus generally more difficult to hydrolyze. Examples of such a polyol includes a neopentyl glycol, a trimethylolpropane, a 1,4-cyclohexanedimethanol, or a combination thereof.

In general embodiments, a polyester-coating comprises a solvent-borne coating. However, a polyester may be suitable for a water-borne coating. A water-borne polyester-coating generally comprises a polyester resin, wherein the acid number of the polyester resin comprises about 40 to about 60, and wherein the acid moieties have been neutralized by an amine, and wherein the coating comprises liquid component comprising a co-solvent. An additional water-borne binder (e.g., an amino resin) may be used to produce thermosetting film formation. In specific aspects, a water-borne polyester-coating produces a film of excellent hardness, gloss, flexibility, or a combination thereof.

In alternative embodiments, a polyester temporary coating (e.g., a non-film forming coating) may be produced, for example, by selection of a polyester comprising fewer or no cross-linkable moiety(s), selection of an additional binder comprising fewer or no cross-linkable moiety(s), reducing the concentration of the polyester and/or the additional binder, or a combination thereof.

c. Modified Cellulose Binders

In some embodiments, a chemically modified cellulose molecule (“modified cellulose,” “cellulosic”) may be used as a coating component (e.g., a binder). Cellulose comprises a polymer of anhydroglucose monomers that may be insoluble in water and organic solvents. Various chemically modified forms of a cellulose with enhanced solubility have been used as a coating component. Examples of chemically modified cellulose (“modified cellulose,” “cellulosic”) include a cellulose ester, a nitrocellulose, or a combination thereof. Examples of a cellulose ester include a cellulose acetate (“CA”), a cellulose butyrate, a cellulose acetate butyrate (“CAB″), a cellulose acetate propionate (“CAP”), a hydroxy ethyl cellulose, a carboxy methyl cellulose, a cellulose acetobutyrate, an ethyl cellulose, or a combination thereof. A cellulose ester coating typically produces a film with excellent flame resistance, toughness, clarity, or a combination thereof. In certain embodiments, a cellulose ester coating may be selected as a topcoat, a clear coating, a lacquer, or a combination thereof. A cellulose ester may be selected for embodiments wherein the coating comprises an automotive coating, a furniture coating, a wood surface coating, a cable coating, or a combination thereof. A thermoplastic coating, a thermosetting coating, or a combination thereof, may comprise a cellulose ester coating.

A cellulose ester may be selected by the properties associated with the degree and/or type of esterification. Typically, solubility in a liquid component and/or combinability with an additional binder may be increased by partial esterification of an anhydroglucose's hydroxy moiety(s). For example, for a cellulose acetate butyrate, properties such as compatibility, diluent tolerance, flexibility (e.g., lower T_{g}), moisture resistance, solubility, or a combination thereof, increases with greater butyrate esterification. However, decreased hydroxyl content alters properties in a cellulose ester. For example, a cellulose acetate butyrate comprising a hydroxy content of about 1% or below has limited solubility in many solvents, while a hydroxy content of about 5% or greater allows solubility in many alcohols, and the increased number of hydroxy moieties allows a greater degree of cross-linking reaction(s) with a binder such as, for example, an amino binder, an acrylic binder, a urethane binder, or a combination thereof. A cellulose acetate butyrate acrylic-coating may be selected as a lacquer, an automotive coating, a coating comprising a metallic pigment (e.g., an aluminum), or a combination thereof. A cellulose acetate butyrate acrylic-coating may comprise a liquid component comprising greater amounts of an aromatic hydrocarbon solvent with the selection of a CAB with greater butyrate ester content. Though not a cellulosic, sucrose esters may be similarly used as cellulose ester, particularly a CAB.

In some embodiments, in a cellulose ester comprising an acetyl ester (e.g., a cellulose acetate, a cellulose acetate butyrate, a cellulose acetate propionate), the acetyl content may range from about 0.1% to about 40.5% acetate. In certain aspects, the acetyl content of a cellulose acetate, a cellulose acetate butyrate, and/or a cellulose acetate propionate may range from about 39.0% to about 40.5%, about 1.0% to about 30.0%, or about 0.3% to about 3.0%, respectively. In many aspects, in a cellulose ester comprising a butyryl ester (e.g., a cellulose acetate butyrate), the butyryl content may range from about 15.0% to about 55.0% butyryl. In other aspects, in a cellulose ester comprising a propionyl ester (e.g., a cellulose acetate propionate), the propionyl content may range from about 40.0% to about 47.0% propionyl. In other embodiments, the hydroxyl content of a cellulose acetate, a cellulose acetate butyrate, and/or a cellulose acetate propionate may range from about 0% to about 5.0%.

A nitrocellulose (“cellulose nitrate”) resin comprises a cellulose molecule wherein a hydroxyl moiety has been nitrated. A nitrocellulose for use in a coating typically comprises an average of about 2.15 to about 2.25 nitrates per anhydroglucose monomer, and may be soluble in an ester, a ketone, or a combination thereof. Additionally, nitrocellulose may be soluble in a combination of a ketone, an ester, an alcohol and/or a hydrocarbon. A nitrocellulose may be selected as a lacquer, an automotive primer, automotive topcoat, a wood topcoat, or a combination thereof. A nitrocellulose coating are typically a thermoplastic coating.

Standard procedures for determining physical and/or chemical properties (e.g., acetyl content, ash, apparent acetyl content, butyryl content, carbohydrate content, carboxyl content, color and haze, combined acetyl, free acidity, heat stability, hydroxyl content, intrinsic viscosity, solution viscosity, moisture content, propionyl content, sulfur content, sulfate content, metal content), of a cellulose and/or a modified cellulose (e.g., a cellulose acetate, a cellulose acetate propionate, a cellulose acetate butyrate, a methylcellulose, a sodium carboxymethylcellulose, an ethylcellulose, a hydroxypropyl methylcellulose, a hydroxyethylcellulose, a hydroxypropylcellulose) have been described, for example, in “ASTM Book of Standards, Volume 06.03, Paint—Pigments, Drying Oils, Polymers, Resins, Naval Stores, Cellulosic Esters, and Ink Vehicles,” D1695-96 D817-96, D871-96, D1347-72, D1439-97, D914-00, D2363-79, D2364-01, D5400-93, D1343-95, D1795-96, D2929-89, D3971-89, D4085-93, D1926-00, D4794-94, D3876-96, D3516-89, D5897-96, D5896-96, D6188-97, D1348-94, and D1696-95, 2002. Specific procedures for determining purity/properties of a nitrocellulose (e.g., nitrogen content) have been described, for example, in “ASTM Book of Standards, Volume 06.03, Paint—Pigments, Drying Oils, Polymers, Resins, Naval Stores, Cellulosic Esters, and Ink Vehicles,” D301-95 and D4795-94, 2002.

In alternative embodiments, a modified cellulose temporary coating (e.g., a non-film forming coating) may be produced, for example, by selection of a modified cellulose comprising fewer or no cross-linkable moiety(s), selection of an additional binder comprising fewer or no cross-linkable moiety(s), reducing the concentration of the modified cellulose and/or additional binder, or a combination thereof.

d. Polyamide and Amidoamine Binders

A polyamide (“fatty nitrogen compound,” “fatty nitrogen product”) comprises a reaction product of a polyamine and a dimerized and/or a trimerized fatty acid. In typical embodiments, a polyamide comprises an oligomer. An amide resin comprises a terminal amine moiety capable of cross-linking with an epoxy moiety, and a polyamide binder may be combined with an epoxide binder. In other aspects, a polyamide may be considered an additive (e.g., a curing agent, a hardening agent, a coreactant) of an epoxide coating. A polyamine-epoxy coating may be used as an industrial coating (e.g., an industrial maintenance coating), a marine coating, or a combination thereof. A polyamide-epoxide coating may be applied to a surface such as, for example, a wood, a masonry, a metal (e.g., a steel), or a combination thereof. However, in some embodiments, a surface may be thoroughly cleaned prior to application to promote adhesion. Such surface preparation in the art may be used, and include, for example, removal of rust, a degraded film, a grease, etc. A polyamide-epoxy coating may comprise a solvent-borne coating. Examples of a solvent for a polyamide include an alcohol, an aromatic hydrocarbon, a glycol ether, a ketone, or a combination thereof. In certain embodiments, a polyamide-epoxy coating may comprise a two-pack coating, wherein a coating component(s) comprising the polyamide resin may be stored in one container, and a coating component(s) comprising the epoxy resin may be stored in a second container. Such a two-pack coating may be admixed immediately before application, as the stoichiometric mix ratio of resin may be formulated to promote a rapid cure. However, in other embodiments, a polyamide-epoxy coating may comprise a single container coating. Such a solvent-borne polyamine-epoxy coating may be formulated for a storage life of a year or more. An aluminum and/or a stainless steel container may be suitable, though a carbon steel container may alter coating and/or film color. However, such a coating typically undergoes film formation in stages, wherein the liquid component may be physically lost by evaporation while thermosetting produces a physically durable film in about 8 to about 10 hours, a chemically resistant film in about three to about four days, and final cross-linking completed in about three weeks. In some embodiments, a polyamine-epoxy coating may undergo chalking upon exterior weathering.

Though a polyamide may be prepared from a fatty acid, it may not be classified as an oil-based binder herein due to the chemistry of film formation for a polyamide binder. The dimerized (“dibasic”) and/or the trimerized fatty acid generally comprises a polyunsaturated fatty acid, a monounsaturated fatty acid, or a combination thereof. In certain aspects, the fatty acid comprises a linseed oil fatty acid, a soybean oil fatty acid, a tall oil fatty acid, or a combination thereof. In specific facets, the fatty acid comprises an 18-carbon fatty acid. However, to reduce the volatile organic compounds of solvent-borne coating, a polyamide binder may be partly or fully substituted, such as about 0% to about 100% substitution, with an amidoamine binder. An amidomine binder differs from a polyamide binder by the use of a fatty acid rather than a dimerized fatty acid in the synthesis of the resin. The selection of the polyamine in the preparation of a polyamide may affect the properties of the polyamide. The polyamine may be linear (e.g., diethylenetriamine), branched and/or cyclic (e.g., aminoethylpiperazine). Standards for physical properties, chemical properties, and/or procedures for testing the purity/properties (e.g., amine value) of a polyamide and/or an amidoamine are described, for example, in “ASTM Book of Standards, Volume 06.03, Paint—Pigments, Drying Oils, Polymers, Resins, Naval Stores, Cellulosic Esters, and Ink Vehicles,” D2071-87, D2073-92, D2082-92, D2072-92, D2074-92, D2075-92, D2076-92, D2077-92, D2078-86, D2079-92, D2080-92, D2081-92, and D2083-92, 2002.

In general embodiments, a polyamine comprises a polyethylene amine. A polyamide produced from a diethylenetriamine may be prepared to comprise a varying amount, typically about 35% to about 85%, of an imidazoline moiety. In other embodiments, the amount of amine moiety capable of cross-linking with an epoxy moiety may vary from about 100 to about 400 amine value. However, the amine value may be converted into units known as “active hydrogen equivalent weight,” which varies from about 550 to about 140, for comparison to the epoxy resins epoxide equivalent weight for determining the stoichiometric mix ratio of a polyamide-epoxy combination. The stoichiometric mix ratio affects coating and/or film properties. As the polyamide to epoxy stoichiometric mix ratio increases from a ratio of less than one to a ratio of greater than one, properties such as excellent impact resistance, excellent chemical resistance, or a combination thereof, decrease while film flexibility increases. Examples of polyamide to epoxy stoichiometric mix ratio include about 2:1 to about 1:2.

In alternative embodiments, a polyamide and/or an amidoamine temporary coating (e.g., a non-film forming coating) may be produced, for example, by selection of a polyamide and/or an amidoamine comprising fewer or no cross-linkable moiety(s), selection of an additional binder comprising fewer or no cross-linkable moiety(s), reducing the concentration of the polyamide and/or an amidoamine and/or an additional binder, selection of a stoichiometric ratio that may be less suitable for a cross-linking reaction, or a combination thereof.

e. Amino Resins

An amino resin (“amino binder,” “aminoplast,” “nitrogen resin”) comprises a reaction product of formaldehyde, an alcohol and a nitrogen compound such as, for example, a urea, a melamine (“1:3:5 triamino triazine”), a benzoguanamine, a glucoluril, or a combination thereof. An amino resin may be used in a thermosetting coating. An amino resin comprises an alkoxymethyl moiety capable of cross-linking with a hydroxyl moiety of an additional binder such as an acrylic binder, an alkyd resin, a polyester binder, or a combination thereof, and in certain embodiments an amino resin may be combined with a binder comprising a hydroxyl moiety in a coating. In some aspects wherein the coating comprises an amino resin and an alkyd resin, the amino:alkyd resin ratio comprises about 1:1 to about 1:5. An amino resin coating may comprise a solvent-borne coating. Examples of a solvent for an amino resin include an alcohol (e.g., a butanol, an isobutanol, a methanol, an isopropanol), a ketone, a hydroxyl functional glycol ether, or a combination thereof. Additionally, an amino resin generally possesses limited solubility in a hydrocarbon (e.g., a xylene), which may be added to a solvent-borne coating's liquid component. In certain aspects, an amino resin coating may be a water-borne coating, wherein water comprises a solvent for an amino resin comprising a plurality of methylol moieties. In other embodiments, a water-borne amino resin coating may comprise a water-reducible coating, particularly wherein the liquid component comprises a glycol ether, an alcohol, or a combination thereof. In certain embodiments, an amino coating comprises an acid catalyst.

An amino resin coating may be cured by baking at a temperature of about 82° C. and about 204° C. Baking generally promotes reactions between amino resin(s), though it does improve the reaction rate between an amino resin and an additional binder. In some embodiments wherein the coating comprises an additional binder, the additional resin comprises less hydroxyl moiety(s) and/or the amino resin comprises a polar amino resin (e.g., a conventional amino resin) when cured by baking than embodiments wherein an acid catalyst may be used. An amino resin coating undergoes rapid film formation, typically lasting about 30 seconds to about 30 minutes, wherein a higher temperature and/or acid catalyst shortens film formation time. An amino resin prepared from a urea may undergo film formation faster than an amino resin prepared from melamine. However, an amino resin coating generally produces an alcohol (e.g., a methanol, a butanol) and formaldehyde during film formation as a byproduct.

An amino resin for use in a coating may be classified by content of a liquid component (e.g., a solvent) as a high solids amino resin or a conventional amino resin. The liquid component may be used to reduce the viscosity of the resin for coating preparation. A high solids amino resin comprises about 80% to about 100%, by weight, an amino resin, with the balance a liquid component. A high solids amino resin may be less polar, less polymeric, lower in viscosity, or a combination thereof, relative to a conventional amino resin. The lower viscosity allows the use of little or no liquid component. Additionally, a high solids amino resin may be water-soluble and/or water reducible. A conventional amino resin comprises less than about 80% amino resin, by weight, with the balance a liquid component. Properties of a high solids and/or a conventional amino resin selected for use in a coating, such as the amount of amino resin and liquid component, the amount of unreacted formaldehyde in the resin preparation, the viscosity of the resin, and/or the ability of the resin to accept additional liquid component as a solvent, may be empirically determined (see, for example, “ASTM Book of Standards, Volume 06.03, Paint—Pigments, Drying Oils, Polymers, Resins, Naval Stores, Cellulosic Esters, and Ink Vehicles,” D4277-83, D1545-98, D1979-97, and D1198-93, 2002; and “ASTM Book of Standards, Volume 06.01, Paint—Tests for Chemical, Physical, and Optical Properties; Appearance,” D2369-01e1, 2002).

In embodiments wherein an amino resin coating comprises an amino resin prepared from a urea, the coating may be used as a wood coating (e.g., a furniture coating), an industrial coating (e.g., an appliance coating), an automotive primer, a clear coating, or a combination thereof. However, an amino resin film, wherein the resin was prepared from a urea, generally produces a film with poor resistance to moisture, and may be used in an internal coating and/or as a part of a multicoat system. In certain embodiments, an amino resin prepared from a melamine, generally produces a film with good resistance to moisture, temperature, UV irradiation, or a combination thereof. A melamine-based amino coating may be applied to a metal surface. In specific aspects, an automotive coating, a coil coating, a metal container coating, or a combination thereof, may comprise such a melamine amino resin coating. In embodiments wherein an amino resin coating comprises an amino resin prepared from a benzoguanamine, the film produced generally possesses poor weathering resistance, good corrosion resistance, water resistance, detergent resistance, flexibility, hardness, or a combination thereof. A benzoguanamine amino resin may be used as an industrial coating, particularly for an indoor application (e.g., an appliance coating). In embodiments wherein an amino resin coating comprises an amino resin prepared from a glycoluril, a higher baking temperature and/or an acid catalyst may be used during film formation, but less byproduct(s) may be released. A glycoluril-based amino-coating typically produces a film with excellent corrosion resistance, humidity resistance, or a combination thereof. A glycoluril-based amino-coating may be selected as a metal coating.

In alternative embodiments, an amino resin temporary coating (e.g., a non-film forming coating) may be produced, for example, by selection of an amino resin that comprising fewer or no cross-linkable moiety(s), selection of an additional binder comprising fewer or no cross-linkable moiety(s), reducing the concentration of the amino resin and/or an additional binder, selection of a binder ratio that may be less suitable for a cross-linking reaction, using a bake cured amino resin coating at temperatures less than may be used for curing (e.g., ambient conditions), or a combination thereof.

f. Urethane Binders

A urethane binder (“polyurethane binder,” “urethane,” “polyurethane”) comprises a binder prepared from compounds that comprise an isocyanate moiety. The urethane binder's urethane moiety may form intermolecular hydrogen bonds between urethane binder polymers, and these non-covalent bonds confer useful properties in a coating and/or a film comprising a urethane binder. The hydrogen bonds may be broken by mechanical stress, but may reform, thereby conferring a property of abrasion resistance. Additionally, a urethane binder may form some hydrogen bonds with water, conferring a plasticizing property to the coating. In certain embodiments, a urethane binder comprises an isocyanate moiety. The isocyanate moiety may be reactive (e.g., cross-linkable) with a moiety comprising a chemically reactive hydrogen. Examples of a chemically reactive hydrogen moiety include a hydroxyl moiety, an amine moiety, or a combination thereof. Examples of an additional binder include a polyol, an amine, an epoxide, a silicone, a vinyl, a phenolic, or a combination thereof. In certain embodiments, a urethane coating comprises a thermosetting coating. In specific aspects, a urethane coating comprises a catalyst (e.g., a dibutyltin dilaurate, a stannous octoate, a zinc octoate). In specific facets, the coating comprises about 10 to about 100 parts per million catalyst. In some embodiments, such a coating undergoes film formation at ambient conditions and/or slightly greater temperatures. A binder comprising an isocyanate moiety may be selected to produce a coating with durability in an external environment. A urethane coating typically possesses good flexibility, toughness, abrasion resistance, chemical resistance, water resistance, or a combination thereof. An aliphatic urethane coating may be selected for the additional property of good lightfastness.

In general embodiments, a urethane binder may be selected based on the materials used in its preparation, which typically affect the urethane binder's properties. An example of a urethane binder includes an aromatic isocyanate urethane binder, an aliphatic isocyanate urethane binder, or a combination thereof. An aliphatic isocyanate urethane binder may be selected for embodiments wherein an improved exterior durability, color stability, good lightfastness, or a combination thereof, relative to an aromatic isocyanate binder, may be desired. Examples of an aliphatic isocyanate urethane binder includes a hydrogenated bis(4-isocyanatophenyl)methane (“4,4′dicyclohexylmethane diisocyanate,” “HMDI”), a HDI, a combination of a 2,2,4-trimethyl hexamethylene diisocyanate and a 2,4,4-trimethyl hexamethylene diisocyanate (“TMHDI”), a 1,4-cyclohexane diisocyanate (“CHDI”), an isophorone diisocyanate (“3-isocyanatomethyl-3,5,5-trimethylcyclohexyl isocyanate,” “IPDI”), or a combination thereof. In certain aspects, a HDI derived binder may be prepared from excess HDI reacted with water, known as “HDI biuret.” In certain aspects, a HDI derived binder may be prepared from a 1,6-hexamethylene diisocyanate isocyanurate, wherein such a HDI derived binder produces a coating with generally improved heat resistance and/or exterior durability may be desired relative to an other HDI derived binder. Standards for physical properties, chemical properties, and/or procedures for testing the purity/properties of urethane precursor component(s) (e.g., a toluene) and urethane resin(s) (e.g., an isocyanate moiety) for use in a coating are described, for example in “ASTM Book of Standards, Volume 06.04, Paint—Solvents; Aromatic Hydrocarbons,” D5606-01, 2002; and “ASTM Book of Standards, Volume 06.03, Paint—Pigments, Drying Oils, Polymers, Resins, Naval Stores, Cellulosic Esters, and Ink Vehicles,” D3432-89 and D2572-97, 2002.

In certain embodiments, a urethane coating comprises a urethane binder capable of a self-cross-linking reaction. An example comprises a moisture-cure urethane, which comprises an isocyanate moiety.

Contact between an isocyanate moiety and a water molecule produces an amine moiety capable of bonding with an isocyanate moiety of another urethane binder molecule in a linear polymerization reaction. In certain aspects, a moisture cure urethane coating may be baked at about 100° C. to about 140° C., to promote a cross-linking reaction between the linear polymers. In certain embodiments, a moisture-cure urethane coating comprises a solvent-borne coating. In specific aspects, a moisture-cure urethane coating comprises a dehydrator. In general aspects, a moisture-cure urethane coating may comprise an one-pack coating, prepared for storage of the coating in anhydrous conditions.

In certain embodiments, a urethane coating comprises a blocked isocyanate urethane binder, wherein the isocyanate moiety has been chemically modified by a hydrogen donor to be inert until contacted with a baking temperature. Such a blocked isocyanate urethane coating may comprise an one-pack coating, as it may be designed for stability at ambient conditions. Additionally, a powder coating may comprise a blocked isocyanate urethane coating.

In certain embodiments, a urethane coating comprises an additional binder. In certain embodiments, a urethane may be combined with a binder such as an amine, an epoxide, a silicone, a vinyl, a phenolic, a polyol, or a combination thereof, wherein the binder comprises a reactive hydrogen moiety. In specific embodiments, selection of a second binder to cross-link with the urethane binder affects coating and/or film properties. In certain aspects, a coating comprising a urethane and an epoxide, a vinyl, a phenolic, or a combination thereof produces a film with good chemical resistance. In other aspects, a coating comprising a urethane and a silicone produces a coating with good thermal resistance. In some aspects, a coating comprises a urethane and a polyol. A primary hydroxyl moiety, secondary hydroxyl moiety, and tertiary hydroxyl moiety of a polyol are respectively the fastest, moderate, and slowest to react with a urethane. Steric hindrance from a neighboring moiety may slow the reaction with a hydroxyl moiety. In an additional example, use of a polyol may increase flexibility of a urethane coating. Often, a selected polyol has a molecular weight from about 200 Da to about 3000 Da. Generally, a lower molecular weight polyol increases the hardness property, lowers the flexibility property, or a combination thereof, of a urethane polyol film. Examples of a polyol include a glycol, a triol (e.g., a 1,4-butane-diol, a diethylene glycol, a trimethylolpropane), a tetraol, a polyester polyol, a polyether polyol, an acrylic polyol, a polylactone polyol, or a combination thereof. Examples of a polyether polyol include a poly(propylene oxide) homopolymer polyol, a poly(propylene oxide), an ethylene oxide copolymer polyol, or a combination thereof.

In certain embodiments, a urethane binder comprises a thermoplastic urethane binder. Typically, a thermoplastic urethane binder comprises from about 40 kDa to about 100 kDa. In particular aspects, a thermoplastic urethane binder comprises little or no isocyanate moiety(s). In general aspects, a thermoplastic urethane coating comprises a solvent borne coating. In specific facets, a thermoplastic urethane coating comprises a lacquer, a high gloss coating, or a combination thereof.

In certain embodiments, a urethane binder comprises a urethane acrylate (“acrylated urethane”) binder. A urethane acrylate binder generally comprises an acrylate moiety at an end of the polymeric binder. The acrylate moiety may be part of an acrylate monomer, wherein the monomer comprises a hydroxyl moiety (e.g., a 2-hydroxy-ethyl acrylate). A urethane acrylate coating generally comprises another binder for cross-linking reaction(s). Examples of a suitable binder include a triacrylate (e.g., a teimethylolpropane). A urethane acrylate coating generally also comprises a viscosifier, wherein the viscosifier reduces viscosity. Examples of such a viscosifer include an acrylate monomer, a N-vinyl pyrrolidone, or a combination thereof. A urethane acrylate coating may be cured by irradiation. Examples of irradiation include UV light, electron beam, or a combination thereof. In embodiments wherein a curing agent comprises an UV light, a urethane acrylate coating typically comprises a photoinitiator. Examples of a suitable initiator include a 2,2,-diethoxyacetophenone, a combination of a benzophenone and an amine synergist, or a combination thereof. In specific facets, a urethane acrylate coating may be applied to a plastic surface. In other facets, a urethane acrylate coating comprises a floor coating, an electronic circuit board coating, or a combination thereof.

In alternative embodiments, a urethane temporary coating (e.g., a non-film forming coating) may be produced, for example, by selection of a urethane resin that comprising fewer or no cross-linkable moiety(s), selection of an additional binder comprising fewer or no cross-linkable moiety(s), reducing the concentration of the urethane resin and/or an additional binder, using a bake cured urethane resin coating at temperatures less than may be used for curing (e.g., ambient conditions), selection of a size range for a thermoplastic urethane resin coating that may be less suitable for film formation (e.g., about 1 kDa to about 40 kDa), or a combination thereof.

i. Water-Borne Urethanes

The previous discussion of a urethane coating(s) focused on solvent-borne urethane coating(s). A water-borne urethane coating typically comprises a water-dispersible urethane binder such as a cationic modified urethane binder and/or an anionic modified urethane binder. A cationic modified urethane binder comprises a urethane binder chemically modified by a diol comprising an amine, such as, for example, a diethanolamine, a methyl diethanolamine, a N,N-bis(hydroxyethyl)-α-aminopyridine, a lysine, a N-hydroxyethylpiperidine, or a combination thereof. An anionic modified urethane binder comprises a urethane binder chemically modified by a diol comprising a carboxylic acid such as a dimethylolpropionic acid (2,2-bis(hydroxymethyl) propionic acid), a dihydroxybenzoic acid, a sulfonic acid (e.g., 2-hydroxymethyl-3-hydroxy-propanesulfonic acid), or a combination thereof.

ii. Urethane Powder Coatings

A urethane powder coating refers to a polyester and/or an acrylic coating, wherein the binder has been modified to comprise a urethane moiety. Such a coating may be a thermosetting, a bake cured coating, an industrial coating (e.g., an appliance coating), or a combination thereof.

g. Phenolic Resins

A phenolic resin (“phenolic binder,” “phenolic”) comprises a reaction product of a phenolic compound and an aldehyde. A type of aldehyde comprises a formaldehyde, and such a phenolic resin may be known as a “phenolic formaldehyde resin” (“PF resin”). The properties of a phenolic resin are affected by the phenolic compound and reaction conditions used during synthesis. A resole resin (“resole phenolic”) may be prepared by a reaction of a molar excess of a phenolic compound with a formaldehyde under alkaline conditions. A novolac resin (“novolac phenolic”) may be prepared by a reaction of a molar excess of a formaldehyde with a phenolic compound under acidic conditions. Examples of a phenolic compound used in preparing a phenolic resin include a phenol; an orthocresol (“o-cresol”); a metacresol, a paracresol (“p-cresol”); a xylenol (e.g., 4-xylenol); a bisphenol-A [“2,2-bis(4-hydroxylphenyl) propane”; “diphenylol propane”); a p-phenylphenol; a p-tert-butylphenol; a p-tert-amylphenol; a p-tert-octyl phenol; a p-nonylphenol; or a combination thereof. Standards for physical properties, chemical properties, and/or procedures for testing the purity/properties of various compounds used in a phenolic resin (e.g., a bisphenol A, a phenol, a cresol, a formaldehyde) for use in a coating are described, for example in “ASTM Book of Standards, Volume 06.04, Paint—Solvents; Aromatic Hydrocarbons,” D6143-97, D3852-99, D4789-94, D2194-02, D2087-97, D2378-02, D2379-99, D2380-99, D1631-99, D6142-97, D4493-94, D4297-99, and D4961-99, 2002. Standards for physical properties, chemical properties, and/or procedures for testing the purity/properties of phenolic resins for use in a coating are described, for example in “ASTM Book of Standards, Volume 06.03, Paint—Pigments, Drying Oils, Polymers, Resins, Naval Stores, Cellulosic Esters, and Ink Vehicles,” D1312-93, D4639-86, D4706-93, D4613-86 and D4640-86, 2002.

In alternative embodiments, a phenolic resin temporary coating (e.g., a non-film forming coating) may be produced, for example, by selection of a phenolic resin comprising fewer or no cross-linkable moiety(s), selection of an additional binder comprising fewer or no cross-linkable moiety(s), reducing the concentration of the phenolic resin and/or the additional binder, using a bake cured phenolic resin coating at temperatures less than may be used for curing (e.g., ambient conditions), or a combination thereof.

i. Resole

A solvent-borne phenolic formaldehyde (e.g., a resole resin) coating typically comprises an alcohol, an ester, a glycol ether, a ketone, or a combination thereof, as a PF solvent. However, a phenolic resin prepared from a phenolic compound comprising an alkyd moiety, such as, for example, a p-tert-butylphenol, a p-tert-amylphenol, a p-tert-octyl phenol, or a combination thereof, typically has solubility in an aromatic compound and/or able to tolerate an aliphatic diluent. Often, a phenolic-resin coating comprises an additional binder such as an alkyd resin, an amino resin, a blown oil, an epoxy resin, a polyamide, a polyvinyl resin [e.g., poly(vinyl butyral)], or a combination thereof. An example of a phenolic-resin coating includes a varnish, an industrial coating, or a combination thereof. A phenolic resin-coating may be selected for embodiments wherein a film possessing solvent resistance, corrosion resistant, of a combination thereof, may be desired. Examples of a surface wherein such property(s) are often used include a surface of a metallic container (e.g., a can, a pipeline, a drum, a tank), a coil coating, or a combination thereof. In specific aspects, a phenolic coating produces a film about 0.2 to about 1.0 mil thick. In specific aspects, coating comprising a phenolic-binder and an additional binder undergoes a thermosetting cross-linking reaction between the binder(s) during film formation. In certain embodiments, a phenolic-resin coating undergoes cure by baking, such as, for example, at about 135° C. to about 204° C. In specific aspects, a baking cure time comprises about one minute to about four hours, with shorter cure times at high temperatures. A phenolic-resin film generally possesses excellent hardness property (e.g., glass-like), excellent resistance to solvents, water, acids, salt, electricity, heat resistance, as well as thermal resistance up to about 370° C. for a period of minutes.

However, a phenolic-resin film may be poorly resistant to alkali unless made from a coating that also comprised an epoxy binder. In certain embodiments, a phenolic-epoxy coating comprises a binder ratio of about 15:85 to about 50:50 phenolic binder:epoxy binder. In certain aspects, a phenolic-epoxy coating possesses flexibility, toughness, or a combination thereof relative to a phenolic coating. In specific facets, a phenolic-epoxy coating may be cured at about 200° C. for about 10 to about 12 minutes.

In other aspects, a phenolic coating comprises a blown oil, an alkyd, or a combination thereof. In some aspects, such a coating comprises a phenolic resin prepared from a p-tert-butylphenol, a p-tert-amylphenol, a p-tert-octyl phenol, or a combination thereof. In specific aspects, such a coating may be applied to an electrical coil, an electrical equipment, or a combination thereof.

ii. Novolak

In other aspects, wherein a film may be desired, a novolak coating may be used. However, a novolak resin may be a non-film forming resin. In some specific aspects, such a coating comprises an epoxy resin. In some facets, the coating comprises a basic catalyst. A film produced from such a novolak-epoxy coating typically possesses good resistance to chemicals, water, heat, or a combination thereof. In specific facets, a high solids coating, a powder coating, a pipeline coating, or a combination thereof, may comprise a novolak-epoxy coating.

A novolak resin prepared from phenolic compound comprising an alkyd moiety such as a p-tert-butylphenol, a p-tert-amylphenol, a p-tert-octyl phenol, or a combination thereof, typically has solubility in an oil. Additionally, a PF resin may be modified by reaction with an oil to produce an oil modified PF resin, which may be oil soluble. An alkyd phenol-formaldehyde resin and/or an oil modified phenol-formaldehyde resin may comprise a non-film forming resin. A coating capable of producing a film may be formulated by combining such a resin with a drying oil, an alkyd, or a combination thereof. In specific aspects, an alkyd phenol-formaldehyde resin, an oil modified phenol-formaldehyde resin undergoes cross-linking with an oil and/or an alkyd. Such a coating may further comprise a liquid component (e.g., a solvent), a drier, a UV absorber, an anti-skinning agent, or a combination thereof. In certain facets, such a coating undergoes film formation under ambient conditions and/or by baking. In particular aspects, such a coating comprises a varnish, a wood coating, or a combination thereof. In specific facets, such a coating comprises a pigment.

h. Epoxy Resins

An epoxy resin (“epoxy binder,” “epoxy”) comprises a compound comprising an epoxide (“oxirane”) moiety. An epoxide resin may be used in a thermosetting coating, a thermoplastic coating, or a combination thereof. An epoxide coating may comprise a solvent borne coating, though examples of a water-borne and/or a powder epoxy coating are described herein. An epoxide coating generally possesses excellent properties of adhesion, corrosion resistance, chemical resistance, or a combination thereof. An epoxide coating may be selected for various surfaces, particularly a metal surface.

An epoxide resin (e.g., a bisphenol A epoxy resin) generally comprises one or two epoxide moiety(s) per resin molecule. An epoxide resin may additionally comprise a monomer, an oligomer, and/or a polymer of repeating chemical units, each generally lacking an epoxide moiety, but comprising a hydroxy moiety. The number of monomer(s) present may be expressed as “n” value, wherein an average increase of one monomer per epoxide resin molecule increases the n value by one. The chemical and/or physical properties of an epoxide resin are affected by the n value. For example, as the n value increases, the chemical reactions selected for film formation in a thermosetting coating may become more dominated by reactions with the increasing numbers of hydroxyl moiety(s), and less dominated by the epoxide moiety(s). Often, an epoxide resin may be classified by an epoxide equivalent weight, which refers to the grams of resin required to provide 1 M epoxide moiety equivalent. In certain embodiments, the epoxide equivalent weight comprises about 182 to about 3050. Additionally, an epoxide resin may be used in a thermoplastic coating, particularly wherein the n value comprises greater than about 25. In certain embodiments, an epoxide resin may possess a n value of about 0 to about 250. Standards for physical properties, chemical properties, and/or procedures for testing the purity/properties of epoxy resins (e.g., epoxy moiety content) for use in a coating are described, for example in “ASTM Book of Standards, Volume 06.03, Paint—Pigments, Drying Oils, Polymers, Resins, Naval Stores, Cellulosic Esters, and Ink Vehicles,” D4142-89, D1652-97, D1726-90, D1847-93, and D4301-84, 2002.

An epoxide moiety may be chemically reactive with another moiety, such as, for example, an amine, a carboxyl, a hydroxyl, and/or a phenol. An epoxide coating may comprise an additional binder capable of undergoing a cross-linking reaction with the epoxide during film formation. Various such additional binders in the art are often referred to as a “curing agent” or “hardener.” The selection of a curing agent and/or an epoxide may affect whether the coating undergoes film formation at ambient conditions and/or by baking.

In alternative embodiments, an epoxide resin temporary coating (e.g., a non-film forming coating) may be produced, for example, by selection of an epoxide resin comprising fewer or no cross-linkable moiety(s), selection of an additional binder comprising fewer or no cross-linkable moiety(s), reducing the concentration of the epoxide resin and/or the additional binder, using a bake cured an epoxide resin at temperatures less than may be used for curing (e.g., ambient conditions), not irradiating the coating, or a combination thereof.

i. Ambient Condition Curing Epoxies

In certain embodiments, a curing agent suitable for curing at ambient conditions comprises an amine moiety such as a polyamine adduct, which comprises an epoxy resin modified to comprise an amine moiety, a polyamide, a ketimine, an aliphatic amine, or a combination thereof. Examples of an aliphatic amine include an ethylene diamine (“EDA”), a diethylene triamine (“DETA”), a triethylene tetraamine (“TETA”), or a combination thereof. Selection of a polyamine adduct generally produces a film with excellent solvent resistance, corrosion resistance, acid resistance, flexibility, impact resistance, or a combination thereof. Selection of a polyamide generally produces a film with improved adhesion, particularly to a moist and/or poorly prepared surface, good solvent resistance, excellent corrosion resistance, good acid resistance, improved flexibility retention, improved impact resistance retention, or a combination thereof. A ketimine comprises a reaction product of a primary amine and a ketone, and produces a coating and/or a film with similar properties as a polyamine and/or an amine adduct. However, the pot life may be longer with a ketimine, and moisture (e.g., atmospheric humidity) activates this cure agent. Examples of an epoxide selected for curing at ambient conditions includes a low mass epoxide resin with a n value from about 0 to about 2.0. In certain embodiments, an epoxy resin may be selected with an epoxy equivalent weight of about 182 to about 1750. In specific aspects, the greater the n value of an epoxide resin, the longer the pot life in a two-pack coating, the greater the coating leveling property, the lower the film solvent resistance, the lower the film chemical resistance, the greater the film flexibility, or a combination thereof. In certain aspects, an ambient curing epoxide coating comprises a two-pack coating, wherein the epoxide resin may be in one container and the curing agent in a second container. In typical aspects, the pot life upon admixing the coating components may comprise about two hours to about two days. An ambient cure epoxide may be selected for an industrial coating (e.g., an industrial maintenance coating), a marine coating, an aircraft primer, a pipeline coating, a HIPAC, or a combination thereof.

ii. Bake Curing Epoxies

In other embodiments, a curing agent suitable for curing by baking includes an amino resin (e.g., a urea melamine-based amino resin, a melamine-based amino resin), a phenolic resin, or a combination thereof. Since baking may be used to promote film formation, an epoxy coating comprising such a curing agent may comprise an one-pack coating. In certain embodiments, an epoxy resin may be selected with an epoxy equivalent weight of about 1750 to about 3050. An epoxy resin coating comprising an amino resin cure agent may be selected for a lower cure temperature. Such a coating may be selected as a can coating, a metal coating, an industrial coating (e.g., equipment, appliances), or a combination thereof. An epoxy coating comprises a phenolic resin cure agent typically possesses greater chemical resistance and/or solvent resistance, and may be selected for a can coating, a pipeline coating, a wire coating, an industrial primer, or a combination thereof. Examples of an epoxide selected for curing by baking includes a higher mass epoxide resins with a n value from about 9.0 to about 12.0. In certain embodiments, a heat-cured epoxy coating comprises a water-borne coating. Such a water-borne coating comprises a higher mass epoxide resin modified to comprise a terpolymer comprising monomers of a styrene, a methacrylic, an acrylate, or a combination thereof, and an amino resin, a phenolic resin, or a combination thereof. Such a water-borne coating may be selected as a can coating.

iii. Electrodeposition Epoxies

Another example of a water-borne epoxide coating comprises an electrodeposition epoxy coating. In certain embodiments, an epoxy resin may be selected with an epoxy equivalent weight of about 500 to about 1500. An anionic and/or a cationic epoxy resin may be electrically attracted to a surface for application. The surface removed from the coating bath, and the coating may be baked cured into a film upon the surface. Such a water-borne coating may be selected for an automotive primer, described elsewhere herein.

iv. Powder Coating Epoxies

A powder coating may comprise an exoxy coating, wherein the various nonvolatile coating components are admixed. Examples of typical admixed components include an epoxy resin, a curing agent, and a pigment, an additive, or a combination thereof. In certain embodiments, an epoxy resin may be selected with an epoxy equivalent weight of about 550 to about 750. The mixture may be then melted, cooled, and powderized. The powder coating may be applied by attraction to an electrostatic charge of a surface. The thermosetting coating may be cured by baking. An epoxy powder coating may be selected as a pipe coating, an electrical devise coating, an industrial coating (e.g., appliance coating, automotive coating, furniture coating), or a combination thereof.

v. Cycloaliphatic Epoxies

A cycloaliphatic epoxy binder possesses a ring structure, rather than the linear structure for the epoxy embodiments described above. Examples of a cycloaliphatic epoxide comprises an ERL-4221 (“3,4-epoxycyclohexylmethyl 3,4-epoxycyclohexane carboxylate”), which has an epoxy equivalent weight of about 131 to about 143, a bis(3,4-epoxycyclohexylmethyl) adipate, which has an epoxy equivalent weight of about 190 to about 210, a 2-(3,4-epoxycyclohexyl-5,5-spiro-3,4-epoxy)cyclohexane-m-dioxane, which has an epoxy equivalent weight of about 133 to about 154, a 1-vinyl-epoxy-3,4-epoxycyclohexane, which has an epoxy equivalent weight of about 70 to about 74, or a combination thereof. Usually, a cycloaliphatic epoxy coating may be combined with another binder, such as a polyol, a polyol modified to comprise a carboxyl moiety, or a combination thereof. An acid may be used to initiate cross-linking, particularly with a polyol. A cycloaliphatic epoxy polyol coating may comprise a triflic acid salt (e.g., diethylammonium triflate) to produce an one-pack coating with a pot life of up to about eight months. In certain embodiments, a cycloaliphatic epoxy coating comprises a UV radiation cured coating, wherein the coating comprises a compound that converts to a strong acid upon UV irradiation (e.g., an onium salt). In certain aspects, a UV radiation cured cycloaliphatic epoxy coating comprises an one-pack coating. A UV radiation cured cycloaliphatic epoxy coating generally possesses excellent flame resistance, water resistance, or a combination thereof, and may be selected as a can coating and/or an electrical equipment coating. A compound comprising a carboxyl moiety (e.g., a carboxyl modified polyol) readily cross-links with a cycloaliphatic epoxy binder. However, such a cycloaliphatic epoxy coating comprising such an additional binder generally has a short pot life (e.g., less than eight hours). In certain aspects, a cycloaliphatic epoxy carboxylic acid binder coating comprises a two-pack coating. A cycloaliphatic epoxy carboxylic acid polyol coating generally possesses excellent adhesion, toughness, gloss, hardness, solvent resistance, or a combination thereof.

i. Polyhydroxyether Binders

A polyhydroxyether binder (“polyhydroxyether resin,” “phenoxy binder,” “phenoxy”) chemically resembles a bisphenol A epoxy resin, though a polyhydroxyether binder lacks an epoxide moiety, and about 30 kDa in size. A thermoplastic coating may comprise a polyhydroxyether. The polyhydroxyether binder comprises a hydroxyl moiety, and may be cross-linked with an additional binder such as an epoxide, a polyurethane comprising an isocyanate moiety, an amino resin, or a combination thereof. A thermosetting polyhydroxyether coating typically possesses excellent physical resistance properties, excellent chemical resistance, modest solvent resistance, or a combination thereof. In alternative embodiments, a polyhydroxyether binder temporary coating (e.g., a non-film forming coating) may be produced, for example, by selection of a polyhydroxyether binder comprising fewer or no cross-linkable moiety(s), selection of an additional binder comprising fewer or no cross-linkable moiety(s), reducing the concentration of the polyhydroxyether binder and/or the additional binder, or a combination thereof.

j. Acrylic Resins

An acrylic resin (“acrylic polymer,” “acrylic binder,” “acrylic”) binder comprises a polymer of an acrylate ester monomer, a methacrylate ester monomer, or a combination thereof. An acrylic-coating generally possesses an improved property of water resistance and/or exterior use durability than a polyester-coating. Other properties that an acrylic-coating typically possesses include color stability, chemical resistance, resistance to a UV light, or a combination thereof. An acrylic resin may further comprise an additional monomer to confer a property to the resin, a coating and/or a film. For example, a styrene, a vinyltoluene, or a combination thereof, generally improves alkali resistance. Examples of such properties include the acrylic resin's chemical reactivity (e.g., cross-linkability), acidity, alkalinity, hydrophobicity, hydrophilicity, T_{g}, or a combination thereof. However, a thermoplastic acrylic film generally possesses poor solvent (e.g., acetone, toluene) resistance. Like other coating produced thermoplastic films, a coating produced thermoplastic acrylic film may be easy to repair by application of additional acrylic coating to an area of solvent damage. An acrylic-coating may be suitable for various surfaces (e.g., metal), and examples of such coatings include an aerosol lacquer, an automotive coating, an architectural coating, a clear coating, a coating for external environment, an industrial coating, or a combination thereof. An acrylic resin may be used to prepare a thermoplastic coating, a thermosetting coating, or a combination thereof. In certain aspects, an acrylic-coating may be selected for use as a thermosetting coating, particularly in embodiments for use upon a metal surface. Acrylic resins generally are soluble in a solvent with a similar solubility parameter. Examples of solvents typically used to dissolve an acrylic resin include an aromatic hydrocarbon (e.g., toluene, a xylene); a ketone (e.g., methyl ethyl ketone), an ester, or a combination thereof.

The thermoplastic and/or thermosetting properties of an acrylic resin are related to the monomers that are comprised in the selected resin. Examples of an acrylate ester monomer include a butylacrylate, an ethylacrylate (“EA”), ethylhexylacrylate (“EHA”), or a combination thereof. Examples of a methacrylate ester monomer include a butylmethacrylate (“BMA”), an ethylmethacrylate, a methylmethacrylate (“MMA”), or a combination thereof. Standards for physical properties, chemical properties, and/or procedures for empirically determining the purity/properties of various acrylic monomers (e.g., an acrylate ester, a 2-ethylhexyl acrylate, a n-butyl acrylate, an ethyl acrylate, a methacrylic acid, an acrylic acid, a methyl acrylate) include, for example, “ASTM Book of Standards, Volume 06.04, Paint—Solvents; Aromatic Hydrocarbons,” D3362-93, D3125-97, D4415-91, D3541-91, D3547-91, D3548-99, D3845-96, D4416-89, and D4709-02, 2002).

In alternative embodiments, an acrylic resin temporary coating (e.g., a non-film forming coating) may be produced, for example, by selection of an acrylic resin comprising fewer or no cross-linkable moiety(s), selection of an additional binder comprising fewer or no cross-linkable moiety(s), reducing the concentration of the acrylic resin and/or an additional binder, using a bake cured acrylic resin coating at temperatures less than may be used for curing (e.g., ambient conditions), selection of a size range for a thermoplastic acrylic resin coating that may be less suitable for film formation (e.g., about 1 kDa to about 75 kDa), selection of a thermoplastic acrylic resin with a T_{g} that may be lower than the temperature ranges herein and/or about 20° C. lower than the temperature range of use, or a combination thereof.

i. Thermoplastic Acrylic Resins

A strait acrylic resin (“strait acrylic polymer,” “strait acrylic binder”) comprises a homopolymer and/or a copolymer comprising an acrylate ester monomer and/or a methacrylate ester monomer. A strait acrylic resin may be used to formulate a thermoplastic coating, as cross-linking reaction(s) are absent or limited without additional reactive moiety(s) in the monomer(s). Generally, a thermoplastic film produced from an acrylic resin-coating may possess a lower elongation, an increased hardness, an increased tensile strength, greater UV resistance (e.g., chalk resistance), color retention, a greater T_{g}, or a combination thereof, with increasing methacrylate ester monomer content in the acrylic resin. However, the ester of a monomer may comprise various alcohol moieties, and an alcohol moiety of larger size generally reduces the T_{g}. Examples a T_{g} value for a homopolymer strait acrylic resins with the include about −100° C. for a poly(octadecyl methacrylate); about −72° C. for a poly(tetradecyl methacrylate); about −65° C. for a poly(lauryl methacrylate); about −60° C. for a poly(heptyl acrylate); about −60° C. for a poly(n-decyl methacrylate); about −55° C. for a poly(n-butyl acrylate); about −50° C. for a poly(2-ethoxyethyl acrylate); about −50° C. for a poly(2-ethylbutyl acrylate); about −50° C. for a poly(2-ethylhexyl acrylate); about −45° C. for a poly(propyl acrylate); about −43° C. for a poly(isobutyl acrylate); about −38° C. for a poly(2-heptyl acrylate); about −24° C. for a poly(ethyl acrylate); about −20° C. for a poly(n-octyl methacrylate); about −20° C. for a poly(sec-butyl acrylate); about −20° C. for a poly(ethylthioethyl methacrylate); about −10° C. for a poly(2-ethylhexyl methacrylate); about −5° C. for a poly(n-hexyl methacrylate); about −3° C. for a poly(isopropyl acrylate); about 6° C. for a poly(methyl acrylate); about 11° C. for a poly(2-ethylbutyl methacrylate); about 16° C. for a poly(cyclohexyl acrylate); about 20° C. for a poly(n-butyl methacrylate); about 35° C. for a poly(hexadecyl acrylate); about 35° C. for a poly(n-propyl methacrylate); about 43° C. for a poly(t-butyl acrylate); about 53° C. for a poly(isobutyl methacrylate); about 54° C. for a poly(benzyl methacrylate); about 60° C. for a poly(sec-butyl methacrylate); about 65° C. for a poly(ethyl methacrylate); about 79° C. for a poly(3,3,5-trimethylcyclohexylmethacrylate); about 81° C. for a poly(isopropyl methacrylate); about 94° C. for a poly(isobornyl acrylate); about 104° C. for a poly(cyclohexyl methacrylate); about 105° C. for a poly(methyl methacrylate); about 107° C. for a poly(t-butyl methacrylate); and about 110° C. for a poly(phenyl methacrylate). Additionally, an estimated T_{g} of a copolymer comprising one or more monomers of an acrylate and/or a methyacrylate monomer may be made by using the following equation: 1/T_{g}═W₁/T_{g1}+W₂/T_{g2}, wherein W₁ and W₂ are the are the molecular weight ratios of the first and the second monomer, respectively; and wherein T_{g1} and T_{g2} are glass transition temperatures of the first and the second monomer, respectively (Fox, T. G., 1956). For many embodiments (e.g., a solvent-borne coating), a T_{g} of about 40° C. to about 60° C., may be suitable.

The thermoplastic properties of an acrylic resin are also related to the molecular mass of the selected resin. Increasing the polymer size of an acrylic resin promotes physical polymer entanglement during film formation. Typically, a thermoplastic film produced from an acrylic-coating may possess a lower flexibility, an increased exterior durability, an increased hardness, an increased solvent resistance, an increased tensile strength, a greater T_{g}, or a combination thereof, with increasing polymer size of the acrylic resin. However, increasing polymer size of an acrylic resin generally increases viscosity of a solution comprising a dissolved acrylic resin, which may make application to a surface more difficult, such as cobwebbing of coating during spray application and the changes of film properties generally reaches a plateau at about 100 kDa. In many embodiments, an acrylic resin may range in mass from about 75 kDa to about 100 kDa.

Examples of such a thermoplastic acrylic-coating include a lacquer. In specific facets, the lacquer possesses a good, high, and/or spectacular gloss. In specific aspects, such a thermoplastic acrylic-coating further comprises a pigment. In specific aspects, a wetting agent may be less likely to be used in a coating comprising an acrylic resin and a pigment, due to the ease of dispersion of a pigment with an acrylic resin. In certain aspects, a thermoplastic acrylic-coating may be selected to coat a metal surface, a plastic surface, or a combination thereof. However, in particular aspects, a thermoplastic acrylic coating comprises an automotive coating. Such an automotive coating may comprise an acrylic binder with a high temperature T_{g} to produce a film of sufficient durability (e.g., hardness) for external use and contact with heated surfaces. In certain aspects, a thermoplastic acrylic coating comprises a binder with a T_{g} to about 90° C. to about 110° C. In additional aspects, an automotive coating comprises a plasticizer, a metallic pigment, or a combination thereof. In specific aspects, a binder for an automotive coating comprises a methylmethacrylate ester monomer. In specific facets, an automotive coating comprises a poly(methyl methacrylate).

ii. Water-Borne Thermoplastic Acrylic Coatings

The thermoplastic acrylic coatings described above are solvent-borne coatings. In other embodiments, a waterborne coating may comprise a thermoplastic acrylic resin. A water-borne acrylic (“acrylic latex”) may comprise an emulsion, wherein the acrylic binder may be dispersed in the liquid component. In general embodiments, an emulsifier (e.g., a surfactant) promotes dispersion. In certain embodiments, an acrylic latex coating comprises about 0% to about 20% coalescent per weight of binder. In many embodiments, a water-borne acrylic resin may range in mass from about 100 kDa to about 1000 kDa. In certain embodiments, a water-borne acrylic coating comprises an associative thickener (“rheology modifier”), which may enhance flow, brushability, splatter resistance, film build, or a combination thereof. A water-borne acrylic may be selected as an architectural coating. An associative thickener forms a network with acrylic resin latex particles by hydrophobic interactions. A hydroxyethyl cellulose (“NEC”) changes the coating rheology by promoting flocculation, which tends to reduce gloss, flow, or a combination thereof. Selection of an acrylic resin with smaller size, greater hydrophobicity, or a combination thereof, and an associative thickener may produce higher gloss, better flow, lower roller splatter, or a combination thereof.

### I. Architectural Coatings

A flat interior coating typically comprises a vinyl acetate and a lesser amount of an acrylate (e.g., a butyl acrylate) monomer(s), which generally produces a film with suitable scrub resistance. A copolymer of an acrylate and a methacrylate may be selected for a semigloss or gloss coating. In certain embodiments, the acrylate resin has a T_{g} to about 20° C. to about 50° C. In some aspects, such a coating generally possesses good block resistance, good print resistance, or a combination thereof. An acrylic resin comprising a monomer comprising a ureide moiety may be selected for enhanced film adhesion (e.g., to a coated surface), blistering resistance, or a combination thereof. An acrylic resin comprising a styrene monomer may be selected for enhanced film water resistance.

An exterior latex coating typically produces a film with greater flexibility than an interior latex due to temperature changes and/or dimensional movement of a surface (e.g., a wood). In certain embodiments, the acrylic resin has a T_{g} to about 10° C. to about 35° C. The selection of a T_{g} may be influenced by the selection of the amount particulate material (e.g., pigment) in the coating to achieve a particular visual appearance. For example, a higher the pigment volume content (“PVC”) that may be selected to reduce gloss. However, to retain properties such as flexibility, a binder with a lower T_{g} may be selected for combination with the higher PVC. For example, a flat exterior latex coating generally possesses a pigment volume content of about 40% to about 60% and a T_{g} of about 10° C. to about 15° C., respectively. In another example, a semigloss or gloss exterior latex binder of a coating generally possesses a T_{g} of about 20° C. to about 35° C., respectively. In other embodiments, the exterior latex binder particle size may be selected to be relatively small such as about 90 nm to about 110 nm. In certain facets, a smaller latex particle size promotes adhesion of the coating and/or the film, particularly to a surface comprising a degraded (e.g., chalking) film. In certain other embodiments, a larger latex particle size may be selected to increase the coating and/or the film's build (e.g., thickness). In certain aspects, a larger latex particle size ranges from, for example about 325 nm to about 375 nm.

### II. Industrial Coatings

A water-borne thermoplastic acrylic latex industrial coating typically comprises a binder with a T_{g} of about 30° C. to about 70° C. Such a coating may be applied to a metal surface, and thus often further comprises a surfactant, an additive, or a combination thereof, to improve an anti-corrosion property. In specific aspects, the industrial coating comprises an anti-corrosion pigment, an anti-corrosion pigment enhancer, or a combination thereof. In contrast, a water-borne acrylic latex industrial maintenance coating may be similar to an exterior flat architectural coating in selection of binder(s), though the industrial maintenance coating may comprise an anti-corrosion pigment, an anti-corrosion pigment enhancer, and/of other anti-corrosion component(s) for use on a metal surface.

iii. Thermosetting Acrylic Resins

Unless otherwise noted, the following thermosetting acrylic resins and/or coatings are typically solvent-borne coatings. In certain embodiments an acrylic coating comprises a thermosetting acrylic resin. A thermosetting acrylic coating typically possesses improved hardness, improved toughness, improved temperature resistance, improved resistance to a solvent, improved resistance to a stain, improved resistance to a detergent, and/or higher application of solids, relative to a thermoplastic acrylic coating. The average size of a thermosetting acrylic resin may be less than a thermoplastic acrylic resin, which promotes a relatively lower viscosity and/or higher application of solids in a solution comprising a thermosetting acrylic resin. In certain embodiments, a thermosetting acrylic resin may comprise from about 10 kDa to about 50 kDa.

A thermosetting acrylic resin comprises a moiety capable of undergoing a cross-linking reaction. A monomer (e.g., a styrene, a vinyltoluene) may comprise the moiety, and be incorporated into the polymer structure of an acrylic resin during resin synthesis and/or the acrylic resin may be chemically modified after polymerization to comprise a chemical moiety. In additional embodiments, an acrylic resin may be selected to comprise a chemical moiety, such as an amine, a carboxyl, an epoxy, a hydroxyl, an isocyanate, or a combination thereof, to confer a property to the acrylic resin produced. Examples of such properties include the acrylic resin's chemical reactivity (e.g., cross-linkability), acidity, alkalinity, hydrophobicity, hydrophilicity, T_{g}, or a combination thereof. In general embodiments, an acrylic resin comprising a carboxyl moiety, a hydroxyl moiety, or a combination thereof, promotes a cross-linking reaction with another binder. In other embodiments, an acrylic resin may be chemically modified to comprise a methylol and/or a methylol ether group, which may comprise a resin capable of self-cross-linking.

### I. Acrylic-Epoxy Combinations

In certain embodiments, a thermosetting acrylic resin may be combined with an epoxide resin. In general embodiments, an acrylic resin comprising a carboxyl moiety may be selected for cross-linking with an epoxy resin. In specific aspects, an acrylic resin comprises about 5% to about 20% of a monomer comprising a carboxyl moiety, such as of an acrylic acid monomer, a methacrylic acid monomer, or a combination thereof. The carboxyl moiety may undergo a cross-linking reaction with an epoxide resin (e.g., a bisphenol A/epichlorohydrin epoxide resin) during film formation. In certain aspects, an epoxide resin cross-linked with an acrylic resin generally produces a film with good hardness, good alkali resistance, greater solvent resistance to a film, poorer UV resistance, or a combination thereof.

A thermosetting acrylic-epoxy coating may be selected for application to a metal surface. Examples of a surface that an acrylic-epoxy coating may be selected for use include an indoor surface, an indoor metal surface (e.g., an appliance), or a combination thereof. In certain aspects, an epoxide resin cross-linked with an acrylic resin generally produces a film with good hardness, good alkali resistance, greater solvent resistance to a film, poorer UV resistance, or a combination thereof. In some facets, an acrylic resin may be combined with an aliphatic epoxide resin to produce a film with relatively improved UV resistance than a bisphenol A/epichlorohydrin based epoxide resin. In another facet, an acrylic resin polymerized with an allyl glycidyl ether monomer, a glycidyl acrylate monomer, a glycidyl methacrylate monomer, or a combination thereof, may undergo a cross-linking reaction with an epoxide resin during film formation. In specific facets, a film produced from cross-linking an epoxide other than a bisphenol A/epichlorohydrin epoxide resin and an acrylic resin comprising an allyl glycidyl ether monomer, a glycidyl acrylate monomer, a glycidyl methacrylate monomer, or a combination thereof, possesses a relatively improved UV resistance.

In certain embodiments, an acrylic epoxy coating comprises a catalyst to promote cross-linking during film formation. In specific aspects, the catalyst comprises a base such as a dodecyl trimethyl ammonium chloride, a tri(dimethylaminomethyl)phenol, a melamine-formaldehyde resin, or a combination thereof. In other embodiments, an acrylic epoxy coating may be cured by baking at about 150° C. to about 190° C. In particular aspects, a film formation time of an acrylic epoxy coating comprises from about 15 minutes to about 30 minutes. In certain embodiments, a thermosetting coating comprises an acrylic epoxide melamine-formaldehyde coating, wherein an acrylic resin, an epoxide resin and a melamine-formaldehyde resin undergo cross-linking during film formation.

### II. Acrylic-Amino Combinations

In other embodiments, a thermosetting acrylic resin may be combined with an amino resin. In general embodiments, an acrylic resin comprising an acid (e.g., carboxyl) moiety, a hydroxyl moiety, or a combination thereof, may be selected for cross-linking with an amino resin. An acrylic amino coating, wherein the acrylic resin comprises an acid moiety, may be cured by baking at, for example about 150° C. for about 30 minutes. However, an acid moiety acrylic amino coating typically undergoes a greater degree of reactions between amino resins, which reduces properties such as toughness. In specific aspects, an acrylic resin comprises a monomer comprising a hydroxyl moiety such as a hydroxyethyl acrylate (“HEX”), a hydroxyethyl methacrylate (“HEMA”), or a combination thereof. An acrylic amino coating, wherein the acrylic resin comprises a hydroxyl moiety, typically comprises an acid catalyst to promote curing by baking at, for example about 125° C. for about 30 minutes. An acrylic amino coating, wherein the amino resin was prepared from a urea, generally produces a film with lower gloss, less chemical resistance, or a combination thereof, than an amino resin prepared from another nitrogen compound. Selection of a melamine and/or a benzoguanamine based amino coating generally produces a film with excellent weathering resistance, excellent solvent resistance, good hardness, good mar resistance, or a combination thereof, and such an acrylic amino coating may be selected for an automotive topcoat.

### III. Acrylic-Urethane Combinations

In other embodiments, a thermosetting acrylic resin may be combined with a urethane resin. In general embodiments, an acrylic resin comprising an acid moiety, a hydroxyl moiety, or a combination thereof, may be selected for cross-linking with a urethane resin. In specific embodiments, an acrylic resin comprises a hydroxyl moiety, such as, for example, a moiety provided by a HEA monomer, a HEMA monomer, or a combination thereof. Selection of an aliphatic isocyanate urethane (e.g., hexamethylene diisocyanate based) generally produces a film with improved color, weathering, or a combination thereof relative to an other urethane(s). An acrylic urethane coating may comprise a catalyst, such as, for example, a triethylene diamine, a zinc naphthenate, a dibutyl tin-di-laurate, or a combination thereof. An acrylic urethane coating cures at ambient conditions. However, an acrylic urethane coating may comprise a two-pack coating to separate the reactive binders until application. An acrylic urethane coating generally produces a film with good weathering, good hardness, good toughness, good chemical resistance, or a combination thereof. An acrylic urethane coating may be selected an aircraft coating, an automotive coating, an industrial coating (e.g., an industrial maintenance coating), or a combination thereof.

### IV. Water-Borne Thermosetting Acrylics

In other embodiments, a thermosetting acrylic coating may comprise a waterborne coating (e.g., a latex coating). Typically, such a thermosetting acrylic coating comprises an acrylic resin with a hydroxyl moiety, an acid moiety, or a combination thereof. An acrylic resin may further comprise an additional monomer such as a styrene, a vinyltoluene, or a combination thereof. The acrylic resin may be combined in a coating with an amino resin, an epoxy resin, or a combination thereof as previously described. A film produced from a water-borne thermosetting acrylic coating may be similar in properties as a solvent-borne counterpart. Such a coating may be selected for a surface such as a masonry, a wood, a metal, or a combination thereof.

k. Polyvinyl Binders

A polyvinyl binder (“polyvinyl,” “vinyl binder,” “vinyl”) typically comprises a polymer comprising a vinyl chloride monomer, a vinyl acetate monomer, or a combination thereof. A solvent-borne polyvinyl coating may comprise a ketone, ester, a chlorinated hydrocarbon, a nitroparaffin, or a combination thereof, as a solvent. A solvent-borne polyvinyl coating may comprise a hydrocarbon (e.g., an aromatic, an aliphatic) as a diluent. A polyvinyl binder may be insoluble in an alcohol, however, in embodiments wherein a solvent-borne polyvinyl coating comprising an additional alcohol soluble binder, alcohol may comprise about 0% to about 20% of the liquid component. In embodiments wherein solvent-borne polyvinyl coating may be cured by baking, a glycol ether and/or a glycol ester may be used in the liquid component to enhance a rheological property. In other embodiments, the liquid component of a polyvinyl coating may comprise a plasticizer (e.g., a phthalate, a phosphate, a glycol ester), wherein the plasticizer typically comprises about 1 to about 25 parts per hundred parts polyvinyl binder, for a non-plastisol and/or a non-organosol coating. A polyvinyl-coating may be used to prepare a thermoplastic coating, a thermosetting coating, or a combination thereof. In specific aspects, a thermoplastic polyvinyl binder coating possesses a T_{g} of about 50° C. to about 85° C. However, in some aspects, a polyvinyl-coating/film possesses moderate resistance to heat, UV irradiation, or a combination thereof. In specific aspects, a polyvinyl-coating comprises a light stabilizer, a pigment, or a combination thereof. In particular facets, the light stabilizer, the pigment (e.g., a titanium dioxide), or the combination thereof, improves the polyvinyl-coating and/or the film's resistance to heat, UV irradiation, or a combination thereof.

In embodiments wherein a polyvinyl coating comprises a solvent-borne coating, a polyvinyl resin may range in mass from about 2 kDa to about 45 kDa. A typical solvent-borne polyvinyl coating comprises a polyvinyl resin, a liquid component wherein the liquid component comprises a solvent, and/or a plasticizer. A solvent-borne polyvinyl coating may additionally comprise a colorizing agent (e.g., a pigment), a light stabilizer, an additional binder, a cross-linker, or a combination thereof.

A polyvinyl binder typically possesses excellent adhesion for a plastic surface, an acrylic and/or acrylic coated surface, a paper, or a combination thereof. A thermoplastic polyvinyl coating may be selected as a lacquer, a topcoat of a can coating (e.g., a can interior surface coating), or a combination thereof. In some embodiments, a polyvinyl-coating may be selected to produce a film with such properties, for example, as excellent water resistance, excellent resistance to various solvents (e.g., an aliphatic hydrocarbon, an alcohol, an oil), excellent resistance to acid pH, excellent resistance to basic pH, inertness relative to food, or a combination thereof.

In many aspects, a polyvinyl resin comprises a copolymer comprising a combination of a vinyl chloride monomer and a vinyl acetate monomer. Often during resin synthesis (e.g., polymerization), a polyvinyl resin may be prepared to further comprise a monomer with specific chemical moiety(s) to confer a property such as solubility in water, solubility in a solvent, compatibility with another coating component (e.g., a binder), or a combination thereof. In certain embodiments, a polyvinyl resin comprises a monomer comprising carboxyl moiety, a hydroxyl moiety (e.g., a hydroxyalkyl acrylate monomer), a monomer comprising an epoxy moiety, a monomer comprising a maleic acid, or a combination thereof. A carboxyl moiety may confer an increased adhesion property (e.g., excellent adhesion to metal). However, a polyvinyl resin comprising a carboxyl moiety without an active enzyme may be not compatible or have limited compatibility with a basic pigment. A thermosetting polyvinyl coating comprising a polyvinyl binder comprising a carboxyl moiety and/or a polyvinyl binder comprising an epoxy moiety generally possesses one or more excellent physical properties (e.g., flexibility), and may be selected as a coil coating. A hydroxyl moiety may confer cross-linkability, compatibility with another coating component, an increased adhesion property (e.g., good adhesion to aluminum), or a combination thereof. Additionally, after polymer synthesis, a polyvinyl resin may be chemically modified to comprise such a specific chemical moiety. In some embodiments, a polyvinyl resin may be chemically modified to comprise a secondary hydroxyl moiety, an epoxy moiety, a carboxyl moiety, or a combination thereof. A polyvinyl resin comprising a secondary hydroxyl moiety may be combined with another binder such as an alkyd, a urethane, an amino-formaldehyde, or a combination thereof. A thermosetting polyvinyl amino-formaldehyde coating comprising a polyvinyl binder comprising a hydroxyl moiety generally possesses good corrosion resistance, water resistance, solvent resistance, chemical resistance, and may be selected as a can coating, a coating for an interior wood surface, or a combination thereof. Standards for physical properties, chemical properties, and/or procedures for testing the purity/properties of various polyvinyl monomers (e.g., a vinyl acetate) and polyvinyl resins (e.g., polymer components, polymer mass, shear viscosity for a higher mass resin, chlorine content) are described, for example, in “ASTM Book of Standards, Volume 06.04, Paint—Solvents; Aromatic Hydrocarbons,” D2190-97, D2086-02, D2191-97, and D2193-97, 2002; “ASTM Book of Standards, Volume 06.03, Paint—Pigments, Drying Oils, Polymers, Resins, Naval Stores, Cellulosic Esters, and Ink Vehicles,” D4368-89, D3680-89, and D1396-92, 2002; and in “ASTM Book of Standards, Volume 06.01, Paint—Tests for Chemical, Physical, and Optical Properties; Appearance,” D2621-87, 2002.

In alternative embodiments, a polyvinyl resin temporary coating (e.g., a non-film forming coating) may be produced, for example, by selection of a polyvinyl resin comprising fewer or no cross-linkable moiety(s), selection of an additional binder comprising fewer or no cross-linkable moiety(s), reducing the concentration of the polyvinyl resin and/or an additional binder, using a bake cured polyvinyl resin coating at temperatures less than may be used for curing (e.g., ambient conditions), selection of a size range for a plastisol and/or an organisol polyvinyl resin coating that may be less suitable for film formation (e.g., about 1 kDa to about 60 kDa), selection of a polyvinyl resin with T_{g} that may be lower than the temperature ranges herein and/or about 20° C. lower than the temperature range of use, or a combination thereof.

i. Plastisols and Orqanisols

A polyvinyl resin of about 60 kDa to about 110 kDa, may be selected for use as an organosol or a plastisol. A plastisol comprises a coating comprising a vinyl homopolymer binder and a liquid component, wherein the liquid component generally comprises a plasticizer comprising a minimum of about 55 parts or more of plasticizer per hundred parts of homopolymer binder in the coating. In certain embodiments, a plastisol comprises, by weight, about 0% to about 10% of a thinner (e.g., an aliphatic hydrocarbon). A plastisol coating typically comprises an additional vinyl binder. A plastisol may comprise a pigment, however, a low oil absorption pigment may be used to avoid an increase in coating viscosity given the liquid component used for a plastisol.

An organosol may be similar to a plastisol, except the less than about 55 parts of plasticizer per hundred parts of homopolymer binder may be used in the coating. In typical embodiments, the liquid component comprises a weak solvent that may act as a dispersant and/or a thinner (e.g., a hydrocarbon). In typical aspects, the reduced content of plasticizer produced a film with an improved hardness property relative to a plastisol. In additional embodiments, the nonvolatile component of an organisol comprises about 50% to about 55%. An organosol coating typically comprises a second binder. In specific aspects, the second binder comprises a vinyl copolymer, an acrylic, or a combination thereof. In certain aspects, the second binder comprises a carboxyl moiety, a hydroxyl moiety, or a combination thereof. In further aspects, an organisol may comprise a third binder. In specific facets, the third binder comprises an amino resin, a phenolic resin prepared from formaldehyde, or a combination thereof. In additional facets, a second binder comprising a hydroxyl moiety may undergo a thermosetting cross-linking reaction with a third binder. An organisol may comprise a pigment suitable for a polyvinyl coating.

A plastisol or organisol may be cured by baking. In general embodiments, baking comprises at a temperature of about 175° C. to about 180° C. In general embodiments, a plastisol and/or an organisol comprises a heat stabilizer. The heat stabilizer may protect a vinyl binder during baking. Examples of a suitable heat stabilizer include a combination of a metal salt of an organic acid and an epoxidized oil and/or a liquid epoxide binder. However, in an embodiment wherein the plastisol or the organisol comprises a binder comprising a carboxyl moiety, a metal salt may be less likely to be used due to possible gellation of the coating, and may be substituted with a merapto tin and/or a tin ester compound.

In embodiments wherein a plastisol or an organisol comprise a binder with good adhesion properties for a surface such as a binder comprising carboxyl moiety, the plastisol or an organisol may be used as a single layer coating. For example, such an organisol may be selected to coat the end of a can. However, a plastisol and/or an organisol may be part of a multicoat system comprising a primer to promote adhesion. In specific aspects, the primer comprises a vinyl resin comprising a carboxyl moiety. In specific facets, the primer further comprises a thermosetting binder such as an amino-formaldehyde, a phenolic, or a combination thereof, to enhance solvent resistance. In certain facets, a coat layer (e.g., a primer) of a multicoat system possesses good solvent resistance to the plasticizer(s) of the organosol and/or a plastisol coat layer.

ii. Powder Coatings

A polyvinyl binder may be selected for use in a powder coating. Typically, a coating component such as a polyvinyl binder, a plasticizer, a colorizing agent, an additive, or a combination thereof, are admixed to prepare a powder coating. Such a powder coating may be applied by a fluidized bed applicator, a spray applicator, or a combination thereof. In some aspects, the coating component(s) are melted then ground into a powder. Such a powder coating may be applied by an electrostatic spray applicator. The coating may be cured by baking. A polyvinyl powder coating may be selected to coat a metal surface.

iii. Water-Borne Coatings

The previous discussions of polyvinyl coatings focused upon solvent-borne and powder coatings. A polyvinyl binder with a T_{g} of about 75° C. to about 85° C., may be selected for use in a dispersion waterborne coating. The liquid component may comprise a cosolvent such as a glycol ether, a plasticizer, or a combination thereof. Examples of a cosolvent include an ethylene glycol monobutyl ether. The dispersion water-borne polyvinyl coating may be used as described for a solvent-borne polyvinyl coating. In another example, an organisol may be prepared with a plasticizer as a latex coating. Such a latex may be suitable for selection as a primer coating. The latex coating may be cured by baking.

I. Rubber Resins

In certain embodiments, a coating may comprise a rubber resin as a binder. A rubber may be either obtained from a biological source (“natural rubber”), synthesized from petroleum (“synthetic rubber”), or a combination thereof. Examples of synthetic rubber include a polymer of a styrene monomer, a butadiene monomer, or a combination thereof. In alternative embodiments, a rubber temporary coating (e.g., a non-film forming coating) may be produced, for example, by selection of a rubber resin comprising fewer or no cross-linkable moiety(s), selection of an additional binder comprising fewer or no cross-linkable moiety(s), reducing the concentration of the rubber resin and/or additional binder, or a combination thereof.

i. Chlorinated Rubber Resins

In general embodiments, a rubber resin comprises a chlorinated rubber resin, wherein a rubber isolated from a biological source has been chemically modified by reaction with chlorine to produce a resin comprising about 65% to about 68% chlorine by weight. A chlorinated rubber resins generally are in a molecular weight range of about 3.5 kDa to about 20 kDa. A chlorinated rubber coating may comprise another binder, such as, for example, an acrylic resin, an alkyd resin, a bituminous resin, or a combination thereof. In specific aspects, a chlorinated rubber resin comprises about 10% to about 50%, by weight, of the binder when in combination with an acrylic resin, an alkyd resin, or a combination thereof. In general embodiments, a chlorinated rubber coating comprises a solvent-borne coating. In certain aspects, a chlorinated rubber coating comprises a liquid component, such as, for example, a solvent, a diluent, a thinner, a plasticizer, or a combination thereof. A thermoplastic coating may comprise a chlorinated rubber coating. To reduce the T_{g} of a film produced from a chlorinated rubber resin, the liquid component generally comprises a plasticizer. In certain aspects, a chlorinated rubber coating comprises about 30% to about 40%, by weight, of plasticizer. In certain facets, a plasticizer may be selected for water resistance (e.g., hydrolysis resistance) such as a bisphenoxyethylformal. In certain facets, a chlorinated rubber coating comprises a light stabilizer, an epoxy resin, an epoxy plasticizer (e.g., epoxidized soybean oil), or a combination thereof, to chemically stabilize a chlorinated resin, coating and/or a film. In other embodiments, a chlorinated rubber coating comprises a pigment, an extender, or a combination thereof. In particular aspects, the pigment comprises a corrosion resistant pigment. A chlorinated rubber film are generally has good chemical resistance (e.g., acid resistance, alkali resistance), water resistance, or a combination thereof. A coating comprising a chlorinated rubber resins may be used, for example, on surfaces that contact a gaseous, a liquid and/or a solid external environments. Examples of such uses include a coating for an architectural coating (e.g., a masonry coating), a traffic marker coating, a marine coating (e.g., a marine vehicle, a swimming pool), a metal primer, a metal topcoat, or a combination thereof.

ii. Synthetic Rubber Resins

Examples of synthetic rubber include polymers comprising a styrene monomer, a methylstyrene (e.g., α-methylstyrene) monomer, or a combination thereof. A solvent-borne coating may comprise a polystyrene and/or polymethylstyrene coating. Examples of a solvent include an aliphatic hydrocarbon, an aromatic hydrocarbon, a ketone, an ester, or a combination thereof. A polystyrene and/or a polymethylstyrene coating may possess good water resistance, good chemical resistance, or a combination thereof. A polystyrene and/or a polymethylstyrene coating may be selected as a primer, a lacquer, a masonry coating, or a combination thereof. A polystyrene homopolymer has a T_{g} of about 100° C., and in certain embodiments, a polystyrene coating may be bake cured. Standards for physical properties, chemical properties, and/or procedures for testing the purity/properties of a styrene monomer, a methylstyrene monomer, (e.g., an α-methylstyrene), a resin comprising a styrene and/or a methylstyrene monomer, are described, for example, in “ASTM Book of Standards, Volume 06.04, Paint—Solvents; Aromatic Hydrocarbons,” D2827-00, D6367-99, D6144-97, D4590-00, D2119-96, D2121-00, and D2340-96, 2002.

Similar to the variability of T_{g} previously described for a thermoplastic acrylic resin, a styrene copolymer with a lower a T_{g} than a polystyrene and/or other altered properties may be produced from polymerization with a monomer such as a butadiene monomer, an acrylic monomer, a maleate ester, an acrylonitrile, an allyl alcohol, a vinyltoluene, or a combination thereof. For example, a butadiene monomer decreases lightfastness, but confers self-cross-linkability to the resin. In another example, an acrylic resin increases the resin's solubility in an alcohol. In a further example, an allyl alcohol monomer confers cross-linkability in combination with a polyol. In certain embodiments, a styrene-butadiene copolymer resin may be selected. In certain aspects, a styrene-butadiene resin comprises a carboxyl moiety to improve an adhesion property, dispersibility in a liquid component, or a combination thereof. In particular facets, a styrene-butadiene coating comprises an emulsifier to increase dispersion in a liquid component, a light stabilizer, or a combination thereof. A thermosetting coating may comprise a styrene-butadiene coating, due to oxidative cross-linking of a butadiene double bond moiety. However, a styrene-butadiene film may have poor chalking resistance, poor color stability, poor UV resistance, or a combination thereof. A styrene-butadiene coating may be selected as a corrosion resistant primer, a wood primer, or a combination thereof. A styrene-vinnyltoluene-acrylate copolymer coating may be selected for an exterior coating, a traffic marker paint, a metal coating (e.g., a metal lacquer), a masonry coating, or a combination thereof.

m. Bituminous Binders

A bituminous binder (“bituminous”) comprises a hydrocarbon soluble in carbon disulfide, may be black or dark colored, and may be obtained from a bitumen deposit and/or as a product of petroleum processing. A bituminous binder typically may be used in an asphalt, a tar, and/or an other construction materials. However, in certain embodiments, a bituminous binder may be used in a coating, particularly in embodiments wherein good resistance to a chemical such as a petroleum based solvent, an oil, a water, or a combination thereof, may be desired. Examples of a bituminous binder include a coal tar, a petroleum asphalt, a pitch, an asphaltite, or a combination thereof. In certain embodiments, a coal tar and/or a pitch may be combined with an epoxy resin to form a thermosetting coating. Such a coating may be selected as a pipeline coating. In other embodiments, an asphaltite and/or a petroleum asphalt may be selected for use as an automotive coating (e.g., an underbody part coating). An asphaltite and/or a petroleum asphalt coating may further comprise an additional binder such as an epoxy. In certain aspects, an asphaltite and/or a petroleum asphalt coating comprises a solvent-borne coating. In specific aspects, an asphaltite and/or a petroleum asphalt coating comprises a plasticizer. In further aspects, an asphaltite and/or a petroleum asphalt coating comprises a wax to increase abrasion resistance.

In further embodiments, a bituminous coating may be selected as a roof coating. Typically, a bituminous roof coating comprises an extender, a thixotrope, or a combination thereof. Examples of a thixotrope additive include asbestos, a silicon extender, a cellulosic, a glass fiber, or a combination thereof. In some aspects, a bituminous roof coating comprises a solvent-borne coating and/or a water-borne coating. Examples of a solvent that may be selected include a mineral spirit, an aliphatic hydrocarbon (e.g., a naphtha, a mineral spirit), an aromatic solvent (e.g., a xylene, a toluene) or a combination thereof. A bituminous roof coating may be selected as a primer, a topcoat, or a combination thereof. A bituminous roof topcoat typically further comprises a metallic pigment.

In certain aspects, a solvent-borne and/or a water-borne bituminous coating comprises an emulsion comprising water and a bituminous binder. In specific facets, the emulsion further comprises a solvent, an extender (e.g., a silica), an emusifier (e.g., a surfactant), or a combination thereof. The extender typically functions to stabilize the emulsion. In particular facets, the emulsion bituminous coating comprises a roof coating, a road coating, a sealer, a primer, a topcoat, or a combination thereof. In facets wherein an emulsion bituminous coating may be selected as a sealer, an additional binder may be added to increase solvent resistance.

In alternative embodiments, a bituminous temporary coating (e.g., a non-film forming coating) may be produced, for example, by selection of an additional binder comprising fewer or no cross-linkable moiety(s), reducing the concentration of the bituminous resin and/or an additional binder, or a combination thereof.

n. Polysulfide Binders

A polysulfide binder comprises a polymer produced from a reaction of a sodium polysufide, a bis(2-chlorethyl) formal and a 1,2,3-trichloropropane. Typically, a polysulfide binder comprises about 1 kDa to about 8 kDa. A polysulfide binder comprises a thiol (“mercaptan”) moiety capable of cross-linking with an additional binder. A polysulfide may undergo cross-linking by an oxidative reaction with an additional binder comprising a peroxide (e.g., dicumen hydroperoxide), a manganese dioxide, a p-quinonedioxime, or a combination thereof. A polysulfide binder may be cross-linked with a glycidyl epoxide, though a tertiary amine may be used as part of the coating to promote this reaction. A polysulfide may undergo cross-linking with a binder comprising an isocyanate moiety, though the binder may comprise a plurality of isocyanates. A polysulfide film typically possesses excellent UV resistance, good general weatherability properties, good chemical resistance, or a combination thereof.

In alternative embodiments, a polysulfide temporary coating (e.g., a non-film forming coating) may be produced, for example, by selection of an additional binder comprising fewer or no cross-linkable moiety(s), reducing the concentration of the bituminous resin and/or an additional binder, or a combination thereof.

o. Silicone Binders

The previous described binders are molecules based on carbon, and are considered herein as “organic binders.” A silicone binder (“silicone”) comprises a binder molecule based on silicone. Examples of a silicone binder include a polydimethyllsiloxane and a methyltriacetoxy silane, a methyltrimethoxysilane, a methyltricyclorhexylaminosilane, a fluorosilicone, a trifluoropropyl methyl polysiloxane, or a combination thereof. In general embodiments, a silicone binder comprises a cross-reactive silicon moiety, examples of which are described below. A silicone coating may be selected for excellent resistance to irradiation (e.g., UV, infrared, gamma), excellent weatherability, excellent biodegradation resistance, flame resistance, excellent dielectric property, which refers to poor electrical conductivity with little detrimental effect on an electrostatic field, or a combination thereof. In specific aspects, a silicon coating comprises an industrial coating. In particular facets, a silicon coating may be applied to an appliance part, a furnace part, a jet engine part, an incinerator part, and/or a missile part. In other embodiments, a silicon coating comprises an organic binder. In particular aspects, a silicon organic binder coating possesses improved heat resistance to an organic binder coating. In other aspects, the greater the silicon binder to organic binder ratio, the greater the cross-linking reactions, greater film hardness, reduced flexibility, or a combination thereof.

In general embodiments, a silicone coating comprises a thermosetting coating. Often, a silicon coating comprises a multi-pack coating due to a limited pot life when the coating components are admixed. The cross-linking reaction depends upon the binder's specific silicon moiety. A plurality of binders may be used, each comprising one or more cross-linking moiety(s). A binder comprising cross-linking SiOH and HOSi moieties generally comprises a cure agent such as a lead octoate, a zinc octoate, or a combination thereof. In general aspects, the thermosetting SiOH and HOSi silicon coating may be bake cured (e.g., 250° C. for one hour). A binder comprising cross-linking SiOH and HSi moieties typically comprises a tin catalyst. A binder comprising cross-linking SiOH and ROSi moieties, wherein a RO comprises an alkoxy moiety, also typically comprises a tin catalyst. A coating prepared using SiOH and ROSi silicon binder typically further comprises an iron oxide, a glass microballon, or a combination thereof to improve heat resistance. This type of silicon may be selected for a rocket and/or a jet engine parts. A binder comprising cross-linking SiOH and CH₃COOSi moieties may be moisture cured, and typically comprises a tin catalyst (e.g., an organotin compound). A binder comprising cross-linking SiOH and R₂NOSi moieties, wherein a R₂NO comprises an oxime moiety, may be also moisture cured, and typically comprises a tin catalyst. The moisture cured silicon coatings may be selected for one-pack silicon coating, though film formation may be slower than other types of a silicon thermosetting coating. A binder comprising cross-linking SiCH═CH₂ and R₂NOSi moieties, wherein a R₂NO comprises an oxime moiety, typically comprises a platinum catalyst, and may be bake cured. A film produced by a SiCH═CH₂ and R₂NOSi silicon coating possesses excellent toughness, flame resistance, or a combination thereof. Such a coating may be selected for a rocket part. However, coating components such as a rubber, a tin compound (e.g., an organotin), or a combination thereof, may inhibit platinum catalyzed film formation in this type of a silicon coating.

In certain embodiments, a silicone coating comprises a solvent-borne coating. Examples of liquid components that may function as a silicon solvent include a chlorinated hydrocarbon (e.g., a 1,1,1-trichloroethane), an aromatic hydrocarbon (e.g., a VMP naphtha, a xylene), an aliphatic hydrocarbon, or a combination thereof. A silicone binder may be insoluble and/or poorly soluble in an oxygenated compound such as an alcohol, a ketone, or a combination thereof, of relatively low molecular weight (e.g., an ethanol, an isopropanol, an acetone). However, a fluorosilicone, which comprises a silicone binder comprising a fluoride moiety, may be combined with a liquid component comprising a ketone such as a methyl ethyl ketone, a methyl isobutyl ketone, or a combination thereof. A fluorosilicone binder may be selected for producing a film with excellent solvent resistance. A silicon coating often comprises a pigment. In specific embodiments, a pigment comprises a zinc oxide, a titanium dioxide, a zinc orthotitanate, or a combination thereof, which may improve a film's resistance to extreme temperature variations, such as those of outerspace. In specific embodiments, a silicon coating may comprise a silica extender (e.g., fumed silica), which often increases durability.

In certain embodiments, a silicon binder comprises a trifluoropropyl methyl polysiloxane binder. In certain aspects, a trifluoropropyl methyl polysiloxane binder may be selected for producing a film with excellent resistance to a petroleum (e.g., an automotive fuel, an aircraft fuel), but poor resistance to an acid or an alkali, particularly at baking conditions.

In alternative embodiments, a silicon temporary coating (e.g., a non-film forming coating) may be produced, for example, by selection of an additional binder comprising fewer or no cross-linkable moiety(s), reducing the concentration of the silicon resin and/or an additional binder, using a bake-cured silicon coating at non-baking conditions, inclusion of a rubber, a tin compound (e.g., an organotin), or a combination thereof.

2. Liquid Components

A liquid component comprises a chemical composition in a liquid state (e.g., a liquid state while comprised in a coating, a film). A liquid component may be added to a coating formulation, for example, to improve a rheological property for ease of application, alter the period of time that thermoplastic film formation occurs, alter an optical property (e.g., color, gloss) of a film, alter a physical property of a coating (e.g., reduce flammability) and/or a film (e.g., increase flexibility), or a combination thereof.

Often a liquid component comprises a volatile liquid that may be partly or fully removed (e.g., evaporated) from the coating during film formation. In many embodiments, about 0% to about 100%, of the liquid component may be lost during film formation. Examples of a volatile liquid include a volatile organic compound (“VOC”), water, or a combination thereof. A coating traditionally comprises one or more solvents that evaporate into the atmosphere after application and are classified as VOCs. A VOC may be an environmental concern due to reactions with atmospheric nitrogen oxides to form ozone. Environmental Protection Agency (“EPA”) findings have linked ground level ozone to increased asthmatic and respiratory conditions in humans. Even short-term exposure to very low levels of ozone may cause chest pain, coughing, nausea, throat irritation, congestion, and reduced lung capacity. In addition, ozone may exacerbate cardiac and lung conditions such as bronchitis, asthma, pneumonia, emphysema, and heart disease. In view of the detrimental effect of ozone, the EPA imposes restrictions on the maximum VOC content permissible in coatings. The coatings industry has proactively reduced use of solvents via several technologies such as powder coatings, ultraviolet cure, high solids, and waterborne coating systems. Various environmental laws and regulations have encouraged the reduction of volatile organic compound(s) use in coatings [see “Paint and Coating Testing Manual, Fourteenth Edition of the Gardner-Sward Handbook,” (Koleske, J. V. Ed.), pp. 3-12, 1995]. As a consequence, a coating may comprise a solvent-borne coating, which typically comprises a VOC and was the coating usually selected prior to enactment of the environmental laws, a high solids coating, which may comprise a solvent-borne coating formulated with a minimum amount of a VOC, a water-borne coating, which comprises water and typically even less VOC, or a powder coating, which comprises little or no VOC. A waterborne coating may be regarded as the closest, environmentally favored alternative to a solvent-based coating, but may be formulated with a solvent (e.g., a cosolvent, a coalescing solvent) to facilitate film formation of a high T_{g} polymer.

In many embodiments, a liquid component may comprise a liquid composition classified based upon function such as a solvent, a thinner, a diluent, a plasticizer, or a combination thereof. A solvent comprises a liquid component used to dissolve one or more components of a material (e.g., a coating). A thinner comprises a liquid component used to reduce the viscosity of a coating, and often additionally confers one or more properties to the coating, such as, for example, dissolving a coating component (e.g., a binder), wetting a colorizing agent, acting as an antisettling agent, stabilizing a coating in storage, acting as an antifoaming agent, or a combination thereof. A diluent comprises a liquid component that does not dissolve a binder.

Liquid components may be classified, based on their chemical composition, as an organic compound, an inorganic compound, or a combination thereof. In many embodiments, an organic compound include a hydrocarbon, an oxygenated compound, a chlorinated hydrocarbon, a nitrated hydrocarbon, a miscellaneous organic liquid component, or a combination thereof. A hydrocarbon comprises one or more carbon and/or hydrogen atoms. Examples of a hydrocarbon include an aliphatic hydrocarbon, an aromatic hydrocarbon, a naphthene, a terpene, or a combination thereof. An oxygenated compound comprises of one or more carbon, hydrogen and/or oxygen atoms. Examples of an oxygenated compound include an alcohol, an ether, an ester, a glycol ester, a ketone, or a combination thereof. A chlorinated hydrocarbon comprises one or more carbon, hydrogen and/or chlorine atoms, but does not comprise an oxygen atom. A nitrated hydrocarbon comprises one or more carbon, hydrogen and/or nitrogen atoms, but does not comprise an oxygen atom. A miscellaneous organic liquid component comprises a liquid other than a chlorinated hydrocarbon and/or a nitrated hydrocarbon comprising one or more carbon, hydrogen and/or other atoms. In certain aspects, a miscellaneous organic liquid component does not comprise an oxygen atom. In typical embodiments, inorganic compounds include an ammonia, a hydrogen cyanide, a hydrogen fluoride, a hydrogen cyanide, a sulfur dioxide, or a combination thereof. However, an inorganic compound generally may be used at temperatures less than ambient conditions, and at pressures greater than atmospheric pressure.

In certain embodiments, a liquid component may comprise an azeotrope. An azeotrope (“azeotropic mixture”) comprises a solution of two or more liquid components at concentrations that produces a constant boiling point for the solution. An azeotrope BP (“A-BP”) refers to the boiling point of an azeotrope. Often, the boiling point (“BP”) of the majority component of an azeotrope may be higher than the A-BP, and in some embodiments, such an azeotrope evaporates from a coating faster than a similar coating that does not comprise the azeotrope. However, in some aspects, a coating comprising an azeotrope with an improved evaporation property may possess a lower flash point temperature, a lower explosion limit, a reduced coating flow, greater surface defect formation, or a combination thereof, relative to a similar coating that does not comprise the azeotrope. Alternatively, an azeotrope may be selected for embodiments wherein a component's BP may be increased. In specific aspects, a coating comprising such an azeotrope may have a relatively slower evaporation rate than a similar coating that does not comprise the azeotrope. In some embodiments, the greater the percentage of liquid component comprises an azeotrope, the greater the conference of an azeotrope's property to a coating. Thus, a specific range of about 50% to about 100%, about 90% to about 100%, and/or about 95% to about 100%, may be sequentially selected in embodiments wherein an azeotrope's property may be desired as a property of a coating.

In some embodiments, a chemically non-reactive (“inert”) liquid component may be selected. Typically, a liquid component may be selected that may be inert relative to a particular chemical reaction to prevent a chemical reaction with an other coating component(s). An example of such a chemical reaction comprises a binder-liquid component reaction that may be inhibitory to a binder-binder film-formation reaction. Examples of a liquid component that are generally inert in an acetal formation reaction include a benzene, a hexane, or a combination thereof. An example of a liquid component that may be inert in a decarboxylation reaction includes a quinoline. Examples of a liquid component that are generally inert in a dehydration reaction include a benzene, a toluene, a xylene, or a combination thereof. An example of a liquid component that may be inert in a dehydrohalogenation reaction includes a quinoline. Examples of a liquid component that are generally inert in a diazonium compound coupling reaction include an ethanol, a glacial acetic acid, a methanol, a pyridine, or a combination thereof. Examples of a liquid component that are generally inert in a diazotization reaction include a benzene, a dimethylformamide, an ethanol, a glacial acetic acid, or a combination thereof. Examples of a liquid component that are generally inert in an esterification reaction include a benzene, a dibutyl ether, a toluene, a xylene, or a combination thereof. Examples of a liquid component that are generally inert in a Friedel-Crafts reaction include a benzene, a carbon disulfide, a 1,2-dichloroethane, a nitrobenzene, a tetrachloroethane, a tetrachloromethane, or a combination thereof. An example of a liquid component that may be inert in a Grignard reaction includes a diethyl ether. Examples of a liquid component that are generally inert in a halogenation reaction include a dichlorobenzene, a glacial acetic acid, a nitrobenzene, a tetrachloroethane, a tetrachloromethane, a trichlorobenzene, or a combination thereof. Examples of a liquid component that are generally inert in a hydrogenation reaction include an alcohol, a dioxane, a hydrocarbon, a glacial acetic acid, or a combination thereof. Examples of a liquid component that are generally inert in a ketene condensation reaction include an acetone, a benzene, a diethyl ether, a xylene, or a combination thereof. Examples of a liquid component that are generally inert in a nitration reaction include a dichlorobenzene, a glacial acetic acid, a nitrobenzene, or a combination thereof. Examples of a liquid component that are generally inert in an oxidation reaction include a glacial acetic acid, a nitrobenzene, a pyridine, or a combination thereof. Examples of a liquid component that are generally inert in a sulfonation reaction include a dioxane, a nitrobenzene, or a combination thereof.

A solvent-borne coating comprises a coating wherein about 50% to about 100%, of a coating's liquid component(s) is not water. Generally, the liquid component of a solvent-borne coating comprises an organic compound, an inorganic compound, or a combination thereof. The liquid component of a solvent-borne coating may function as a solvent, a thinner, a diluent, a plasticizer, or a combination thereof. In certain embodiments, a solvent-borne coating may comprise water. In specific aspects, the water may function as a solvent, a thinner, a diluent, or a combination thereof. The water component of a solvent-borne coating may comprise about 0% to about 49.999% of the liquid component. In certain embodiments, the water component of a water-borne or a solvent-borne coating may be fully or partly miscible in the non-aqueous liquid component. Examples of the percent of water that may be miscible, by weight at about 20° C., in various liquids typically used in solvent-borne coatings include about 0.01% water in a tetrachloroethylene; about 0.02% water in an ethylbenzene; about 0.02% water in a p-xylene; about 0.02% water in a tricholorethylene; about 0.05% water in a 1,1,1-tricholoroethane; about 0.05% water in a toluene; about 0.1% water in a hexane; about 0.16% water in a methylene chloride; about 0.2% water in a dibutyl ether; about 0.2% water in a tetrahydronaphthalene; about 0.42% water in a diisobutyl ketone; about 0.5% water in a cyclohexyl acetate; about 0.5% water in a nitropropane; about 0.6% water in a 2-nitropropane; about 0.62% water in a butyl acetate; about 0.72% water in a dipentene; about 0.9% water in a nitroethane; about 1.2% water in a diethyl ether; about 1.3% water in a methyl tert-butyl ether; about 1.4% water in a trimethylcyclohexanone; about 1.65% water in an isobutyl acetate; about 1.7% water in a butyl glycol acetate; about 1.9% water in an isopropyl acetate; about 2.4% water in a methyl isobutyl ketone; about 3.3% water in an ethyl acetate; about 3.6% water in a cyclohexanol; about 4.0% water in a trimethylcyclohexanol; about 4.3% water in an isophorone; about 5.8% water in a methylbenzyl alcohol; about 6.5% water in an ethyl glycol acetate; about 7.2% water in a hexanol; about 7.5% water in a propylene carbonate; about 8.0% water in a methyl acetate; about 8.0% water in a cyclohexanone; about 12.0% water in a methyl ethyl ketone; about 16.2% water in an isobutanol; about 19.7% water in a butanol; about 25.0% water in a butyl glycolate; and/or about 44.1% water in a 2-butanol.

Various examples of such liquid components are described herein, including properties often used to select a chemical composition for use as a liquid component for a particular coating composition, which may be applied in use in other material formulations and/or another composition described herein. Additionally, standards for physical properties, chemical properties, and/or procedures for testing purity/properties, are described for various types of liquid components (e.g., hydrocarbons, cycloaliphatic hydrocarbons, aromatic hydrocarbons, alcohols, ketones, esters, glycol ethers, mineral spirits, miscellaneous solvents, plasticizers) in, for example, “ASTM Book of Standards, Volume 06.04, Paint—Solvents; Aromatic Hydrocarbons,” D4790-99, D268-01, D3437-99, D1493-97, D235-02, D1836-02, D3735-02, D3054-98, D5309-02, D4734-98, D2359-02, D4492-98, D4077-00, D3760-02, D6526-00, D841-02, D843-97, D5211-01, D5471-97, D5871-98, D5713-00, D852-02, D1685-00, D4735-02, D3797-00, D3798-00, D5135-02, D5136-00, D5060-95, D3193-96, D3734-01, D1152-97, D770-95, D3622-95, D1007-00, D1719-95, D304-95, D319-95, D2635-01, D1969-01, D2306-00, D1612-95, D5008-01, D268-01, D1078-01, D329-02, D1363-94, D740-94, D2804-02, D1153-94, D3329-99, D2917-02, D3893-99, D4360-90, D2627-02, D2916-88, D2192-96, D4614-95, D3545-02, D3131-02, D3130-95, D1718-98, D4615-95, D3540-90, D1617-90, D2634-02, D5137-01, D3728-99, D4835-93, D4773-02, D3128-02, D331-95, D330-93, D4837-02, D4773-02, D4836-95, D5776-99, D5808-95, D5917-02, D6069-01, D6212-99, D6313-99, D6366-99, D6428-99, D6621-00, D6809-02, D5399-95, D6229-01, D6563-00, D6269-98, D3257-01, D847-96, D1613-02, D848-02, D1614-95, D4367-02, D4534-99, D2360-00, D1353-02, D1492-02, D849-02, D3961-98, D1364-02, D3160-96, D1476-02and D1722-98, D853-97, D5194-96, D363-90, D1399-95, D1468-93, D3620-98, D3546-90, and D1721-97, 2002.

a. Solvents, Thinners, and Diluents

A coating may comprise a liquid component that may function as a solvent, a thinner, a diluents, or a combination thereof. In one embodiment of a coating, a particular liquid component may function as a solvent, while in another coating composition comprising, for example, a different binder the same liquid component may function as a thinner and/or a diluent. Whether a liquid component functions primarily as a solvent, a thinner, or a diluent depends considerably upon the particular solvent and/or the rheological property the liquid component confers to a specific coating composition. For example, the ability of the liquid component to function as a solvent, or lack thereof of such ability, relative to the other coating component(s) generally differentiates a solvent from a diluent. A thinner may be primarily included into a coating composition in combination with a solvent and/or a diluent to alter a rheological property such as to reduce viscosity, enhance flow, enhance leveling, or a combination thereof. In addition to the additional techniques in the art to discern such differences of use for a specific liquid composition in a coating, examples of differing solubility properties for specific categories of liquid components, and empirical techniques for determining the solubility properties of a specific liquid component, relative to another coating component, are described herein.

A solute comprises a coating component dissolved by a solvent liquid component. A solute may comprise a solid, a liquid and/or a gas from prior to being dissolved. Solvency (“solvent power”) refers to the ability of a solvent to dissolve a solute, maintain a solute in solution upon addition of a diluent, and reduce the viscosity of a solution. A solvent may be used to produce a solvent-borne coating, wherein the coating possesses particular a rheological property for application to a surface and/or creation of a film of a particular thickness. Additionally, a solvent may contribute to an appearance property, a physical property, a chemical property, or a combination thereof, of a coating and/or a film. In many embodiments, a solvent comprises a volatile component of a coating, wherein about 50% to about 100%, of the solvent may be lost (e.g., evaporates) during film formation. In certain aspects, the rate of solvent loss slows during application and/or film formation. Such a change in solvent loss rate may promote a rheologically related property during application and/or initial film formation, such as ease of application, minimum sag, reduce excessive flow, or a combination thereof, while still promoting a rheologically related property post-application, such as a leveling property, an adhesion property, or a combination thereof.

Depending upon the ability of a liquid component to dissolve, partly dissolve, or unsuccessfully dissolve a coating component, a coating may comprise, a real solution, a colloidal solution and/or a dispersion, respectively. Often the ability of a liquid component to dissolve a coating component may be detrimentally affected by increasing particulate matter size (e.g., pigment size, cell-based particulate material size, etc.) and/or molecular mass of the coating component. For example, a real solution comprises a clear and/or a homogenous liquid solution. In typical embodiments, a real solution may be produced when a potential solute of about 1.0 nm or less in diameter may be combined with a solvent. A colloidal solution comprises a physically non-homogenous solution, which may be a clear to opalescent in appearance. Often, a colloidal solution may be produced when a potential solute of between about 1.0 nm to about 100 nm (“0.1 μm”) in diameter may be combined with a solvent. A dispersion comprises a composition comprising two liquid and/or solid phases, which may be turbid to milky in appearance. Generally, a dispersion may be produced when a potential solute of greater than about 0.1 μm in diameter may be combined with a solvent.

In many aspects, a coating composition may comprise a combination of a real solution, a colloidal solution and/or a dispersion, depending upon the various solubility's of coating components and liquid components. For example, a paint may comprise a real solution of a binder and a liquid component, and a dispersion of a pigment within the liquid component.

Depending upon other coating components, a liquid component may function as an active solvent and/or a latent solvent. An active solvent may be capable of dissolving a solute. Additionally, an active solvent often reduces viscosity of a coating composition. In certain embodiments, an ester, a glycol ether, a ketone, or a combination thereof may be selected for use as an active solvent. A latent solvent, in pure form, does not demonstrate solute dissolving ability. However, the latent solvent may demonstrate the ability to dissolve a solute in a combination of an active solvent and the latent solvent; confer a synergistic improvement in the dissolving ability of an active solvent when combined with the active solvent, or a combination thereof. In certain embodiments, an alcohol may be selected for use as a latent solvent. In certain embodiments, a latent solvent comprises a thinner. A diluent, whether in pure form or in combination with an active solvent and/or a latent solvent, does not demonstrate solute dissolving ability, but may be combined with an active solvent and/or a latent solvent to produce a liquid component with a suitable ability to dissolve a coating component. In certain embodiments, hydrocarbon may be selected for use as a diluent. In particular aspects, a hydrocarbon diluent comprises an aromatic hydrocarbon, an aliphatic hydrocarbon, or a combination thereof. In particular facets, an aromatic hydrocarbon diluent may be selected, due to a generally greater tolerance by a many solvents relative to an aliphatic hydrocarbon. In certain aspects, a diluent may be used to alter a rheological property (e.g., reduce viscosity) of a coating composition, reduce cost of a coating composition, or a combination thereof.

The ability of a solvent to dissolve a potential solute may be related to the intermolecular interactions between the solvent molecules, between the potential solute molecules, between the solvent and the potential solute, as well as the molecular size of the potential solute. Examples of intermolecular interactions include, for example, ionic (“Coulomb”), dipole-dipole (“directional”), ionic-dipole, induction (“permanent dipole/induced dipole”), dispersion (“nonpolar,” “atomic dipole,” “London-Van der Walls”), hydrogen bond, or a combination thereof. The sum of intramolecular interactions for a compound, relevant for the preparation of a solution, is the solubility parameter (“δ”). The solubility parameter comprises a measure of the total energy used to separate molecules of a liquid. Such a separation of molecules of a solvent occurs during the incorporation of the molecules of a solute during the dissolving process. The solubility parameter is the square root of the molar energy of vaporization of a liquid divided by the molar volume of a liquid, measured at about 25° C. Additionally, the solubility parameter may also be expressed as the square root of the sum of the squares of the dispersion (“δ_{d}”), polar (“δₚ”) and hydrogen bond (“δₕ”) solubility parameters.

Often, preparation of a coating composition may be aided by comparing the solubility parameter of a potential solvent and a potential solute (e.g., a binder) to ascertain the theoretical ability of a coating composition comprising a solution to be created. In many embodiments, coating components, wherein at least one coating component comprises a liquid with a solubility parameter that comprises less than an absolute value of about 6, are able to form a solution. The closer this value is to 0, the greater the general ability to form a solution. Additionally, the lower the individual absolute difference (e.g., about six or less) between the dispersion solubility parameters of coating components, the polar solubility parameter of coating components, and/or the hydrogen bond solubility parameter of coating components, the generally greater ability to form a solution. The solubility parameter, dispersion solubility parameter, polar solubility parameter, and hydrogen bond solubility parameter, and methods for determining such values, and additional methods for determining the theoretical ability of coating components to form a solution have been described (see, for example, in “ASTM Book of Standards, Volume 06.03, Paint—Pigments, Drying Oils, Polymers, Resins, Naval Stores, Cellulosic Esters, and Ink Vehicles,” D3132-84, 2002).

However, due to exceptions to the ability of certain liquid components and potential solute coating components to form solutions, empirically determining the ability of a solute to dissolve in a solvent may be used in certain embodiments. Standard techniques in the art may be used for determining the ability of a liquid component comprising one or more liquids to function as an active solvent, a latent solvent, a diluent, or a combination thereof, relative to one or more potential solutes. For example, the solvency of a liquid component comprising an active solvent (e.g., an oxygenated compound), a latent solvent, a diluent (e.g., a hydrocarbon), or a combination thereof, particularly for use in a lacquer coating, may be determined as described in “ASTM Book of Standards, Volume 06.04, Paint—Solvents; Aromatic Hydrocarbons,” D1720-96, 2002). In an additional example, the solvency for a liquid component that primarily comprises a hydrocarbon, and comprises little or lacks an oxygenated compound, may be determined as described in “ASTM Book of Standards, Volume 06.04, Paint—Solvents; Aromatic Hydrocarbons,” D1133-02, 2002). In a further example, the solvency of a solution comprising a liquid component and an additional coating component (e.g., a binder) may be determined, as described in “ASTM Book of Standards, Volume 06.03, Paint—Pigments, Drying Oils, Polymers, Resins, Naval Stores, Cellulosic Esters, and Ink Vehicles,” D1545-98, D1725-62, D5661-95, D5180-93, D6038-96, D5165-93, and D5166-97, 2002. In a supplemental example, the dilutability of a solution comprising liquid component (e.g., a solvent and diluent) and an additional coating component (e.g., a binder) may be determined, as described in “ASTM Book of Standards, Volume 06.03, Paint—Pigments, Drying Oils, Polymers, Resins, Naval Stores, Cellulosic Esters, and Ink Vehicles,” D5062-96, 2002.

In certain embodiments, a liquid component may be selected on the basis of evaporation rate. The evaporation rate of a coating directly affects a physical aspect of film formation caused by loss of a liquid component, as well as the pot life of a coating, such as after opening a coating container. Though the evaporation rate may be known for various pure chemicals, empirical determination of the evaporation rate of a liquid component and/or a coating may be done, as described, for example, in “ASTM Book of Standards, Volume 06.01, Paint—Tests for Chemical, Physical, and Optical Properties; Appearance,” D3539-87, 2002.

Additionally, the boiling point range of a liquid component often may be useful in estimating whether the liquid component evaporates faster or slower relative to another liquid component. Examples of methods for measuring a boiling point for a liquid component (e.g., a hydrocarbon, a chlorinated hydrocarbon) are described in “ASTM Book of Standards, Volume 06.04, Paint—Solvents; Aromatic Hydrocarbons,” D1078-01 and D850-02e1, 2002. The evaporation rate may be also related to the flash point of a liquid component and/or coating. In certain embodiments, a liquid component may be selected on the basis of flash point and/or fire point, which comprises a measure of the danger of use of a flammable coating composition in, for example, storage, application in an indoor environment, etc. A flash point refers to the “lowest temperature at which the liquid gives off enough vapor to form an ignitable mixture with air to produce a flame when a source of ignition is brought close to the surface of the liquid under specified conditions of test at standard barometric pressure (760 mmHG, 101.3KPa),” and a fire point refers to “the lowest temperature at which sustained burning of the sample takes place for at least 5 seconds” [“Paint and Coating Testing Manual, Fourteenth Edition of the Gardner-Sward Handbook” (Koleske, J. V. Ed.), pp. 140 and 142, 1995]. Examples of methods for measuring the flash point and/or fire point for a liquid component and/or a coating are described in and “ASTM Book of Standards, Volume 06.01, Paint—Tests for Chemical, Physical, and Optical Properties; Appearance,” D1310-01, D3934-90, D3941-90, and D3278-96e1, 2002.

Though much or all liquid component(s) may be lost from a coating composition during film formation, a liquid component may still contribute to the visual properties of a coating and/or a film. In embodiments wherein a liquid component may be selected as a colorizing agent, the color and/or darkness of the liquid may be empirically measured (see, for example, “ASTM Book of Standards, Volume 06.04, Paint—Solvents; Aromatic Hydrocarbons,” D1209-00, D1686-96, and D5386-93b, 2002); and “ASTM Book of Standards, Volume 06.01, Paint—Tests for Chemical, Physical, and Optical Properties; Appearance,” D1544-98, 2002. In some embodiments, a liquid component and/or a coating may be selected on the basis of odor (e.g., faint odor, pleasant odor, etc.). A coating and/or a coating component may be evaluated for suitability in a particular application based on odor using, for example, techniques described in “ASTM Book of Standards, Volume 06.04, Paint—Solvents; Aromatic Hydrocarbons,” D1296-01, 2002; and “ASTM Book of Standards, Volume 06.01, Paint—Tests for Chemical, Physical, and Optical Properties; Appearance,” D6165-97, 2002.

i. Hydrocarbons

A hydrocarbon may be obtained as a petroleum, a vegetable product, or a combination thereof. As a consequence of imperfect purification (e.g., distillation) from these sources, a hydrocarbon may comprise a mixture of chemical components. A hydrocarbon may be selected as an active solvent to dissolve an oil (e.g., a drying oil), an alkyd, an asphalt, a rosin, a petroleum, or a combination thereof. A hydrocarbon may be more suitable as a latent solvent and/or a diluent in embodiments to dissolve an acrylic resin, an epoxide resin, a nitrocellulose resin, a urethane resin, or a combination thereof. However, a hydrocarbon may be immiscible in water.

### I. Aliphatic Hydrocarbons

In general embodiments, an aliphatic hydrocarbon may be selected as an active solvent for an alkyd, an oil, wax, a polyisobutene, a polyethylene, a poly(butyl acrylate), a poly(butyl methacrylate), a poly(vinyl ethers), or a combination thereof. In other embodiments, an aliphatic hydrocarbon may be selected as a diluent in combination with an additional liquid component. In alternative embodiments wherein an aliphatic hydrocarbon may be selected as a non-solvent liquid component, a composition comprising a polar binder, a cellulose derivative, or a combination thereof, may be insoluble. An aliphatic hydrocarbon may be selected as a liquid component in embodiments wherein a chemically inert liquid component may be desired. Examples of an aliphatic hydrocarbon include, a petroleum ether, a pentane (CAS No. 109-66-0), a hexane (CAS No. 110-54-3), a heptane (CAS No. 142-82-5), an isododecane (CAS No. 13475-82-6), a kerosene, a mineral spirit, a VMP naphthas, or a combination thereof. A hexane, a heptane, or a combination thereof, may be selected for a coating wherein rapid evaporation of such a liquid component may be desired (e.g., a fast drying lacquer). An example of an azeotrope comprising an aliphatic hydrocarbon includes an azeotrope comprising a hexane. Examples of an azeotrope comprising a majority of a hexane (BP about 65° C. to about 70° C.) include those comprising about 2.5% an isobutanol (azeotrope BP 68.3° C.); about 5.6% water (A-BP 61.6° C.); about 21% an ethanol (A-BP 58.7° C.); about 22% an isopropyl alcohol (A-BP 61.0° C.); about 26.9% a methanol (A-BP 50.0° C.); about 37% a methyl ethyl ketone (A-BP 64.2° C.); and/or about 42% an ethyl acetate (A-BP 65.0° C.).

An aliphatic hydrocarbon may comprise a petroleum distillation product of a heterogeneous chemical composition. Such an aliphatic hydrocarbon may be classified by a physical and/or a chemical property (e.g., boiling point range, flash point, evaporation rate) (see, for example, “ASTM Book of Standards, Volume 06.04, Paint—Solvents; Aromatic Hydrocarbons,” D235-02 and D3735, 2002). In certain embodiments, such a petroleum distillation product aliphatic hydrocarbon may be classified, for example, as a mineral spirit, a VMP naphthas or a kerosene (e.g., deodorized kerosene). A mineral spirit (“white spirit,” “petroleum spirit”) comprises a petroleum distillation fraction with a boiling point between about 149° C. to about 204° C., and a flash point of about 38° C. or greater. A mineral spirit may further be classified as a regular mineral spirit, which possesses the properties previously described for a mineral spirit; a high flash mineral spirit, which possesses a higher minimum flash point (e.g., about 55° C. or greater); a low dry point mineral spirit (“Stoddard solvent”), which typically evaporates about 50% faster than a regular mineral spirit; or an odorless mineral spirit, which generally possesses less odor than a regular mineral spirit, but may also possess relatively weaker solvency property. A mineral spirit may be selected for embodiments wherein a solvent and/or a diluent may be desired for an alkyd coating, a chlorinated rubber coating, an oil-coating, a vinyl chloride copolymer coating, or a combination thereof. A VMP naphtha possess a similar solvency property as a mineral spirit, but evaporates faster with a BP of about 121° C. to about 149° C., and typically has a flash point of about 4° C. or greater. A VMP naphtha may further be classified as a regular VMP naphtha, which possesses the properties previously described for a VMP naphtha; a high flash VMP naphtha, which possesses a higher minimum flash point (e.g., about 34° C. or greater); or an odorless VMP naphtha, which generally possesses less odor than a regular mineral spirit. A VMP naphtha may be selected for a coating that may be spray applied, an industrial coating, or a combination thereof. A petroleum ether comprises a petroleum distillation fraction with a boiling point between about 35° C. to about 80° C., with a low flash point (e.g., about −46° C.), and may be used in embodiments wherein rapid evaporation may be desired.

### II. Cycloaliphatic Hydrocarbons

In embodiments wherein a cycloaliphatic hydrocarbon may be selected as a solvent, a composition comprising an oil, an alkyd, a bitumen, a rubber, or a combination thereof, usually may be dissolved. In alternative embodiments wherein a cycloaliphatic hydrocarbon may be selected as a non-solvent liquid component, a composition comprising a polar binder such as a urea-formaldehyde binder, a melamine-formaldehyde binder, a phenol-formaldehyde binder; a cellulose derivative, such as, a cellulose ester binder; or a combination thereof, may be insoluble. A cycloaliphatic hydrocarbon may be soluble in other organic solvent(s), but not soluble in water. Examples of a cycloaliphatic hydrocarbon include a cyclohexane (CAS No. 110-82-7); a methylcyclohexane (CAS No. 108-87-2); an ethylcyclohexane (CAS No. 1678-91-7); a tetrahydronaphthalene (CAS No. 119-64-2); a decahydronaphthalene (CAS No. 91-17-8); or a combination thereof. A tetrahydronaphthalene may be selected for a coating wherein oxidation of a binder may occur during film formation; a high gloss typically occurs in a film, a smooth surface may be a property in a film, or a combination thereof. An example of an azeotrope comprising a cycloaliphatic hydrocarbon includes an azeotrope comprising a cyclohexane. Examples of an azeotrope comprising a majority of cyclohexane (BP about 80.5° C. to about 81.5° C.) include those comprising about 8.5% water (A-BP 69.8° C.); about 10% a butanol (A-BP 79.8° C.); about 14% an isobutanol (A-BP 78.1° C.); about 20% a propanol (A-BP 74.3° C.); about 37% a methanol (A-BP 54.2° C.); and/or about 40% a methyl ethyl ketone (A-BP 72.0° C.).

### III. Terpene Hydrocarbons

A terpene typically possesses an improved solvency property, stronger odor, or a combination thereof, relative to an aliphatic hydrocarbon. Examples of a terpene includes a wood terpentine oil (CAS No. 8008-64-2); a pine oil (CAS No. 8000-41-7); a α-pinene (CAS No. 80-56-8); a β-pinene; dipentene (CAS No. 138-86-3); a D-limonene (CAS No. 5989-27-5); or a combination thereof. Dipentene may be selected for embodiments wherein an improved solvency property, a slower evaporation rate, or a combination thereof, relative to a turpentine, may be desired. A pine oil may be classified as an oxygenated compound, but may be described under hydrocarbons due to convention in the art. A pine oil generally comprises a terpene alcohol. A pine oil may be selected for embodiments wherein a greater range of solvency for solutes, a slow evaporation rate, or a combination thereof, may be desired. An example of an azeotrope comprising a terpene includes an azeotrope comprising a α-pinene. An example of an azeotrope comprising a majority of α-pinene (BP 154.0° C. to 156.0° C.) includes an azeotrope comprising about 35.5% a cyclohexanol (A-BP 149.9° C.).

A terpene hydrocarbon (“terpene”) may comprise a by-product from pines tree and/or citrus processing of a heterogeneous chemical composition. Such a terpene hydrocarbon (e.g., a terpentine) may be classified by a physical and/or chemical property (see, for example, “ASTM Book of Standards, Volume 06.03, Paint—Pigments, Drying Oils, Polymers, Resins, Naval Stores, Cellulosic Esters, and Ink Vehicles,” D804-02, D13-02, D233-02, D801-02, D802-02, and D6387-99, 2002. Examples of a terpentine include a gum turpentine, a steam-distilled wood turpentine, a sulfate wood turpentine, a destructively distilled wood turpentine, or a combination thereof. Both a gum turpentine and a sulfate wood turpentine generally comprise a combination of a α-pinene and a lesser quantity of a β-pinene. A steam-distilled wood terpentine generally comprises a α-pinene and a lesser component of a dipentene and one or more other terpene(s). Destructively distilled wood turpentine generally comprises various aromatic hydrocarbons and a lesser quantity of one or more terpene(s).

### IV. Aromatic Hydrocarbons

An aromatic hydrocarbon typically possesses a greater solvency property and/or odor relative to other hydrocarbon types. Examples of an aromatic hydrocarbon include a benzene (CAS No. 71-43-2); a toluene (CAS No. 108-88-3; “methylbenzene”); an ethylbenzene (CAS No. 100-41-4); a xylene (CAS No. 1330-20-7); a cumene (“isopropylbenzene”; CAS No. 98-82-8); a type I high flash aromatic naphthas; a type II high flash aromatic naphthas; a mesitylene (CAS No. 108-67-8); a pseudocumene (CAS No. 95-63-6); a cymol (CAS No. 99-87-6); a styrene (CAS No. 100-42-5); or a combination thereof. A xylene typically comprises an o-xylene (CAS No. 56004-61-6); a m-xylene (CAS No. 108-38-3); a p-xylene (CAS No. 41051-88-1); and/or a trace ethylbenzene. A toluene may be selected for embodiments wherein rapid evaporation may be desired. In specific aspects, a toluene may be selected for a spray applied coating, an industrial coating, or a combination thereof. A xylene may be selected for embodiments wherein a moderate evaporation rate may be desired. In specific aspects, a xylene may be selected for an industrial coating. An aromatic hydrocarbon may comprise a petroleum-processing product of heterogeneous chemical composition such as a high flash aromatic naphtha (e.g., a type I, a type II). A type I high flash aromatic naphtha and a type II high flash aromatic naphtha possess a minimum flash point of about 38° C. and about 60° C., respectively. Standards for the characteristic chemical an/or physical property of an aromatic naphtha have been described (see, for example, “ASTM Book of Standards, Volume 06.04, Paint—Solvents; Aromatic Hydrocarbons,” D3734, 2002). A high flash naphtha typically has a slow evaporation rate. In specific embodiments, a high flash aromatic naphtha may be used in an industrial coating, a coating that may be baked, or a combination thereof. An example of a high flash aromatic comprises a Solvesso 100 (CAS No. 64742-95-6). Examples of an azeotrope comprising an aromatic hydrocarbon include an azeotrope comprising a toluene andor a m-xylene. Examples of an azeotrope comprising a majority of a toluene (BP 110° C. to 111° C.) include those comprising about 27% a butanol (A-BP 105.6° C.); and/or about 44.5% an isobutanol (A-BP 100.9° C.). Examples of an azeotrope comprising a majority of a m-xylene (BP 137.0° C. to 142.0° C.) include those comprising about 14% a cyclohexanol (A-BP 143.0° C.); and/or about 40% water (A-BP 94.5° C.).

ii. Oxygenated Compounds

An oxygenated compound (“oxygenated liquid compound,” “oxygenated liquid component”) may be chemically synthesized by standard chemical manufacturing techniques. As a consequence, an individual oxygenated compound may be a homogenous chemical composition, with singular, rather than a range of, chemical and physical properties. The oxygen moiety of an oxygenated compound generally enhances the strength and breadth of solvency for potential solute(s) relative to a hydrocarbon. Additionally, an oxygenated compound typically has some or complete miscibility with water. Examples of an oxygenated compound include an alcohol, an ester, a glycol ether, a ketone, or a combination thereof. A liquid component often comprises a combination of an alcohol, an ester, a glycol ether, a ketone and/or an additional liquid to produce suitable chemical and/or physical properties for a coating and/or a film.

### I. Alcohols

An alcohol comprises an alcohol moiety. However, a typical “alcohol” comprises a single hydroxyl moiety. The alcohol moiety confers miscibility with water. Consequentially, increasing molecular size of an alcohol comprising a single alcohol moiety generally reduces miscibility with water. Alcohols typically possess a mild and/or pleasant odor. An alcohol may be a poor primary solvent, though ethanol may be an exception relative to a solute comprising a phenolic and/or a polyvinyl resin. An alcohol may be selected as a latent solvent, co-solvent, a coupling solvent, a diluent, or a combination thereof such as with solute comprising a nitrocellulose lacquer, a melamine-formaldehyde, a urea formaldehyde, an alkyd, or a combination thereof. Examples of an alcohol include a methanol (CAS No. 67-56-1); an ethanol (CAS No. 64-17-5); a propanol (CAS No. 71-23-8); an isopropanol (CAS No. 67-63-0); a 1-butanol (CAS No. 71-36-3); an isobutanol (CAS No. 78-83-1); a 2-butanol-(CAS No. 78-92-2); a tert-butanol (CAS No. 75-65-0); an amyl alcohol (CAS No. 71-41-0); an isoamyl alcohol (123-51-3); a hexanol (25917-35-5); a methylisobutylcarbinol (CAS No. 108-11-2); a 2-ethylbutanol (CAS No. 97-95-0); an isooctyl alcohol (CAS No. 26952-21-6); a 2-ethylhexanol (CAS No. 104-76-7); an isodecanol (CAS No. 25339-17-7); a cylcohexanol (CAS No. 108-93-0); a methylcyclohexanol (CAS No. 583-59-5); a trimethylcyclohexanol; a benzyl alcohol (CAS No. 100-51-6); a methylbenzyl alcohol (CAS No. 98-85-1); a furfuryl alcohol (CAS No. 98-00-0); a tetrahydrofurfuryl alcohol (CAS No. 97-99-4); a diacetone alcohol (CAS No. 123-42-2); a trimethylcyclohexanol (116-02-9); or a combination thereof. A furfuryl alcohol and/or a tetrahydrofurfuryl alcohol may be selected as a primary solvent for a polyvinyl binder. Examples of an azeotrope comprising an alcohol include an azeotrope comprising a butanol, an ethanol, an isobutanol, and/or a methanol. Examples of an azeotrope comprising a majority of a butanol (BP 117.7° C.) include those comprising about 97% a butanol and about 3% a hexane (A-BP 67° C.); about 32% a p-xylene (A-BP 115.7° C.); about 32.8% a butyl acetate (A-BP 117.6° C.); about 44.5% water (A-BP 93° C.); and/or about 50% an isobutyl acetate (A-BP 114.5° C.). Examples of an azeotrope comprising a majority of an ethanol (BP 78.3° C.) include those comprising about 4.4% water (A-BP 78.2° C.); and/or about 32% toluene (A-BP 76.7° C.). Examples of an azeotrope comprising a majority of an isobutanol (BP 107.7° C.) include those comprising about 2.5% a hexane (A-BP 68.3° C.); about 5% an isobutyl acetate (A-BP 107.6° C.); about 17% a p-xylene (A-BP 107.5° C.); about 33.2% water (A-BP 89.9° C.); and/or about 48% a butyl acetate (A-BP 80.1° C.). An example of an azeotrope comprising a majority of a methanol (BP 64.6° C.) includes an azeotrope comprising about 30% a methyl ethyl ketone (A-BP 63.5° C.).

II. Ketones

A ketone comprises a ketone moiety. However, a typical ketone comprises a single ketone moiety. A ketone generally possesses some miscibility with water, and a strong odor. In general embodiments, a ketone may be selected as a primary solvent, a thinner, or a combination thereof. Examples of a ketone include an acetone (CAS No. 67-64-1); a methyl ethyl ketone (CAS No. 78-93-3); a methyl propyl ketone (CAS No. 107-87-9); a methyl isopropyl ketone (CAS No. 563-80-4); a methyl butyl ketone (CAS No. 591-78-6); a methyl isobutyl ketone (CAS No. 108-10⁻¹); a methyl amyl ketone (CAS No. 110-43-0); a methyl isoamyl ketone (CAS No. 110-12-3); a diethyl ketone (CAS No. 96-22-0); an ethyl amyl ketone (CAS No. 541-85-5); a dipropyl ketone (CAS No. 110-43-0); a diisopropyl ketone (CAS No. 565-80-0); a cyclohexanone (CAS No. 108-94-1); a methylcylcohexanone (CAS No. 1331-22-2); a trimethylcyclohexanone (CAS No. 873-94-9); a mesityl oxide (CAS No. 141-79-7); a diisobutyl ketone (CAS No. 108-83-8); an isophorone (CAS No. 78-59-1); and/or a combination thereof. An acetone may be selected for complete miscibility in water, fast evaporation, or a combination thereof. In certain embodiments, an acetone may be used as a liquid component in an aerosol, a spray-applied coating, or a combination thereof. In specific aspects, an acetone may be used as a thinner. In other aspects, acetone may be used in a coating wherein a nitrocellulose, an acrylic, or a combination thereof, may be dissolved. A methyl ethyl ketone, a methyl isobutyl ketone, and/or an isophorone may be selected in embodiments wherein a fast evaporation rate, moderate evaporation rate, or slow evaporation rate, respectively, may be desired. In specific facets, an isophorone may be selected for a baked coating, an industrial coating, or a combination thereof. Examples of an azeotrope comprising a ketone include an azeotrope comprising an acetone, a methyl ethyl ketone and/or a methyl isobutyl ketone. Examples of an azeotrope comprising a majority of an acetone (BP 56.2° C.) include those comprising about 12% a methanol (A-BP 55.7° C.); and/or about 41% a hexane (A-BP 49.8° C.). Examples of an azeotrope comprising a majority of a methyl ethyl ketone (BP 79.6° C.) include those comprising about 11% a water (A-BP 73.5° C.); about 32% an isopropyl alcohol (A-BP 77.5° C.); and/or about 34% an ethanol (A-BP 74.8° C.). Examples of an azeotrope comprising a majority of a methyl isobutyl ketone (BP 114° C. to 117° C.) include those comprising about 24.3% water (A-BP 87.9° C.); and/or about 30% a butanol (A-BP 114.35° C.).

III. Esters

An ester may comprise an alkyl acetate, an alkyl propionate, a glycol ether acetate, or a combination thereof. An ester generally possesses a pleasant odor. In general embodiments, an ester possesses a solubility property that decreases with increasing molecular weight. A glycol ester acetate typically possesses a slow evaporation rate. In specific aspects, a glycol ester acetate may be selected as a retarder solvent, a coalescent, or a combination thereof. Examples of an ester include a methyl formate (CAS No. 107-31-3); an ethyl formate (CAS No. 109-94-4); a butyl formate (CAS No. 592-84-7); an isobutyl formate (CAS No. 542-55-2); a methyl acetate (CAS No. 79-20-9); an ethyl acetate (CAS No. 141-78-6); a propyl acetate (CAS No. 109-60-4); an isopropyl acetate (CAS No. 108-21-4); a butyl acetate (CAS No. CAS-No. 123-86-4); an isobutyl acetate (CAS No. 110-19-0); a sec-butyl acetate (CAS No. 105-46-4); an amyl acetate (CAS No. 628-63-7); an isoamyl acetate (CAS No. 123-92-2); a hexyl acetate (CAS No. 142-92-7); a cyclohexyl acetate (CAS No. 622-45-7); a benzyl acetate (CAS No. 140-11-4); a methyl glycol acetate (CAS No. 110-49-6); an ethyl glycol acetate (CAS No. 111-15-9); a butyl glycol acetate (CAS No. 112-07-2); an ethyl diglycol acetate (CAS No. 111-90-0); a butyl diglycol acetate (CAS No. 124-17-4); a 1-methoxypropyl acetate (CAS No. 108-65-6); an ethoxypropyl acetate (CAS No. 54839-24-6); a 3-methoxybutyl acetate (CAS No. 4435-53-4); an ethyl 3-ethoxypropionate (CAS No. 763-69-9); an isobutyl isobutyrate (CAS No. 97-85-8); an ethyl lactate (CAS No. 97-64-3); a butyl lactate (CAS No. 138-22-7); a butyl glycolate (CAS No. 7397-62-8); a dimethyl adipate (CAS No. 627-93-0); a glutarate (CAS No. 119-40-0); a succinate (CAS No. 106-65-0); an ethylene carbonate (CAS No. 96-49-1); a propylene carbonate (CAS No. 108-32-7); a butyrolactone (CAS No. 96-48-0); or a combination thereof. An ethylene carbonate and/or a propylene carbonate generally possess a high flash point, a slow evaporation rate, a weak odor, or a combination thereof. An ethylene carbonate may be used for use in a coating at temperatures greater than about 25° C. Examples of an azeotrope comprising an ester include an azeotrope comprising a butyl acetate, an ethyl acetate and/or a methyl acetate. Examples of an azeotrope comprising a majority of a butyl acetate (BP 124° C. to 128° C.) include those comprising about 27% water (A-BP 90.7° C.) and/or about 35.7% an ethyl glycol (A-BP 125.8° C.). Examples of an azeotrope comprising a majority of an ethyl acetate (BP 76° C. to 77° C.) include those comprising about 5% a cyclohexanol (A-BP 153.8° C.); about 8.2% water (A-BP 70.4° C.); about 22% a methyl ethyl ketone (A-BP 76.7° C.); about 23% an isopropyl alcohol (A-BP 74.8° C.); and/or about 31% an ethanol (A-BP 71.8° C.). An example of an azeotrope comprising a majority of a methyl acetate (BP 55.0° C.-57.0° C.) includes an azeotrope comprising about 19% a methanol (A-BP 54° C.).

IV. Glycol Ethers

A glycol ether comprises an alcohol moiety and an ether moiety. The glycol ether generally possesses good solvency, high flash point, slow evaporation rate, mild odor, miscibility with water, or a combination thereof. In some embodiments, a glycol ether may be selected as a coupling solvent, a thinner, or a combination thereof. In particular aspects, a glycol ether may be selected as a liquid component of a lacquer. Examples of a glycol ether include a methyl glycol (CAS No. 109-86-4); an ethyl glycol (CAS No. 110-80-5); a propyl glycol (CAS No. 2807-30-9); an isopropyl glycol (CAS No. 109-59-1); a butyl glycol (CAS No. 111-76-2); a methyl diglycol (111-77-3); an ethyl diglycol (CAS No. 111-90-0); a butyl diglycol (CAS No. 112-34-5); an ethyl triglycol (CAS No. 112-50-5); a butyl triglycol (CAS No. 143-22-6); a diethylene glycol dimethyl ether (CAS No. 111-96-6); a methoxypropanol (CAS No. 107-98-2); an isobutoxypropanol (CAS No. 23436-19-3); an isobutyl glycol (CAS No. 4439-24-1); a propylene glycol monoethyl ether (CAS No. 52125-53-8); a 1-isopropoxy-2-propanol (CAS No. 3944-36-3); a propylene glycol mono-n-propyl ether (CAS No. 30136-13-1); a propylene glycol n-butyl ether (CAS No. 5131-66-8); a methyl dipropylene glycol (CAS No. 34590-94-8); a methoxybutanol (CAS No. 30677-36-2); or a combination thereof. An example of an azeotrope comprising a glycol ether includes an azeotrope comprising an ethyl glycol. An example of an azeotrope comprising a majority of an ethyl glycol (BP 134° C. to 137° C.) includes an azeotrope comprising about 50% a dibutyl ether (A-BP 127° C.).

V. Ethers

Examples of an ether include a diethyl ether (CAS No. 60-29-7); a diisopropyl ether (CAS No. 108-20-3); a dibutyl ether (CAS No. 142-96-1); a di-sec-butyl ether (CAS No. 6863-58-7); a methyl tert-butyl ether (CAS No. 1634-04-4); a tetrahydrofuran (CAS No. 109-99-9); a 1,4-dioxane (CAS No. 123-91-1); a metadioxane (CAS No. 505-22-6); or a combination thereof. A tetrahydrofuran may be selected as a primary solvent for a polyvinyl binder. An example of an azeotrope comprising an ether includes an azeotrope comprising a tetrahydrofuran. An example of an azeotrope comprising a majority of a tetrahydrofuran (BP 66° C.) includes an azeotrope comprising about 5.3% water (A-BP 64.0° C.).

iii. Chlorinated Hydrocarbons

A chlorinated hydrocarbon generally comprises a hydrocarbon, wherein the hydrocarbon comprises a chloride atom moiety. A chlorinated hydrocarbon generally possesses a high degree of non-flammability, and consequently lacks a flash point. A chlorinated hydrocarbon may be selected for embodiments where high flash point may be desired. In particular facets, a chlorinated hydrocarbon may be added to a liquid component to reduce the liquid component's flash point. In certain facets, a chlorinated hydrocarbon may be combined with a mineral spirit, methylene chloride, or a combination thereof, for a reduction of the flash point. In particular aspects, a chlorinated hydrocarbon (e.g., a methylene chloride, a trichloroethylene) may be selected as a solvent for removal of hydrophobic material from a surface (e.g., a grease, an undesired coating and/or film). However, a chlorinated hydrocarbon may be subject to an environmental regulation or law. Examples of a chlorinated hydrocarbon include a methylene chloride (CAS No. 75-09-2; “dichloromethane”); a trichloromethane (CAS No. 67-66-3); a tetrachloromethane (CAS No. 56-23-5); an ethyl chloride (CAS No. 75-00-3); an isopropyl chloride (CAS No. 75-29-6); a 1,2-dichloroethane (CAS No. 107-06-2); a 1,1,1-trichloroethane (CAS No. 71-55-6; “methylchloroform”); a trichloroethylene (CAS No. 79-01-6); a 1,1,2,2-tetrachlorethane (CAS No. 79-55-6); a 1,2-dichloroethylene (CAS No. 75-35-4); a perchloroethylene (CAS No. 127-18-4); a 1,2-dichloropropane (CAS No. 78-87-5); a chlorobenzene (CAS No. 108-90-7); or a combination thereof. A methylene chloride may be selected for embodiments wherein a fast evaporation rate may be desired. A 1,1,1-trichloroethane may be selected for embodiments wherein a photochemically inert liquid component may be desired. Additionally, a methylene chloride may be selected as a coating remover. Examples of an azeotrope comprising a chlorinated hydrocarbon include an azeotrope comprising a methylene chloride, a trichloroethylene and/or a 1,1,1-trichloroethane. Examples of an azeotrope comprising a majority of a methylene chloride (BP 40.2° C.) include those comprising about 1.5% water (A-BP 38.1° C.); about 3.5% an ethanol (A-BP 41.0° C.); and/or about 8% a methanol (A-BP 39.2° C.). Examples of an azeotrope comprising a majority of a trichloroethylene (BP 86.7° C.) include those comprising about 6.6% water (A-BP 72.9° C.); about 27% an ethanol (A-BP 70.9° C.); and/or about 36% a methanol (A-BP 60.2° C.). An example of an azeotrope comprising a majority of a 1,1,1-trichloroethane (BP 74.0° C.) includes an azeotrope comprising about 4.3% water (A-BP 65.0° C.).

iv. Chlorinated Hydrocarbons

A nitrated hydrocarbon comprises a hydrocarbon, wherein the hydrocarbon comprises a nitrogen atom moiety. Examples of a nitrated hydrocarbon include a nitroparaffin, a N-methyl-2-pyrrolidone (“NMP”), or a combination thereof. Examples of a nitroparaffin include a nitroethane, a nitromethane, a nitropropane, a 2-nitropropane (“2NP”), or a combination thereof. A 2-nitropropane may be selected for embodiments as a substitute for a butyl acetate relative to a solvent property, but wherein a greater evaporation rate may be desired. A N-methyl-2-pyrrolidone may be selected for embodiments wherein a strong solvent property, miscibility with water, high flash point, biodegradability, low toxicity, or a combination thereof may be desired. In certain aspects, a N-methyl-2-pyrrolidone may be used in a water-borne coating, a coating remover, or a combination thereof.

v. Miscellaneous Organic Liquids

A miscellaneous organic liquid comprises a liquid comprising carbon that are useful as a liquid component for a coating, but are not readily classified as a hydrocarbon, an oxygenated compound, a chlorinated hydrocarbon, a nitrated hydrocarbon, or a combination thereof. Examples of a miscellaneous organic liquid include a carbon dioxide; an acetic acid, a methylal (CAS No. 109-87-5); a dimethylacetal (CAS No. 534-15-6); a N,N-dimethylformamide (CAS No. 68-12-2); a N,N-dimethylacetamide (CAS No. 127-19-5); a dimethylsulfoxide (CAS No. 67-68-5); a tetramethylene suflone (CAS No. 126-33-0); a carbon disulfide (CAS No. 75-15-0); a 2-nitropropane (CAS No. 79-46-9); a N-methylpyrrolidone (CAS No. 872-50-4); a hexamethylphosphoric triamide (CAS No. 680-31-9); a 1,3-dimethyl-2-imidazolidinone (CAS No. 80-73-9); or a combination thereof. Carbon dioxide may function as a liquid component when prepared under pressure and temperature conditions to form a supercritical liquid. A supercritical liquid has properties between that of a liquid and a gas, and may be used in spray application of a coating wherein the appropriate pressure conditions may be maintained. Supercritical carbon dioxide may be formulated with a coating using the tradename technique Unicarb™ (Union Carbide Chemicals and Plastics Co., Inc.). Supercritical carbon dioxide may be selected as a substitute for a hydrocarbon diluent in embodiments wherein chemical inertness, non-flammability, rapid evaporation, or a combination thereof, may be used. In certain aspects, about 0% to about 30%, of a hydrocarbon liquid component may be replaced with a supercritical carbon dioxide.

b. Plasticizers

In certain embodiments, a coating may comprise a plasticizer. A plasticizer may be selected for embodiments wherein a resin possesses an unsuitable brittleness and/or low flexibility property upon film formation. Properties a plasticizer typically confers to a coating and/or a film include, for example, enhancing a flow property of a coating, lowering a film-forming temperature range, enhancing the adhesion property of a coating and/or a film, enhancing the flexibility property of a film, lowering the T_{g}, improving film toughness, enhancing film heat resistance, enhancing film impact resistance, enhancing UV resistance, or a combination thereof. Since a function of a plasticizer may be to alter a film's properties, many plasticizer's possess a high (e.g., baking temperature) boiling point, as such a compound may be less volatile, with increasing boiling point temperature. In certain aspects, a plasticizer may function as a solvent, a thinner, a diluent, a plasticizer, or a combination thereof, for a coating composition and/or film at a temperature greater than ambient conditions.

A plasticizer may interact with a binder by a polar interaction, but may be chemically inert relative to the binder. A plasticizer typically lowers the T_{g} of a binder below the temperature a coating comprising the binder may be applied to a surface. In many embodiments, a plasticizer have a vapor pressure less than about 3 mm at about 200° C., a mass of about 200 Da to about 800 Da, a specific gravity of about 0.75 to about 1.35, a viscosity of about 50 cSt to about 450 cSt, a flash point temperature greater than about 120° C., or a combination thereof. A plasticizer may comprise an organic liquid (e.g., an ester). Standards for physical properties, chemical properties, and/or procedures for testing purity/properties, are described for plasticizers (e.g., undesired acidity, color, undesired copper corrosion, boiling point, ester content, odor, water contamination) in, for example, “ASTM Book of Standards, Volume 06.04, Paint—Solvents; Aromatic Hydrocarbons,” D1613-02, D1209-00, D849-02, D1078-01, D1617-90, D1296-01, D608-90, and D1364-02, 2002; and “ASTM Book of Standards, Volume 06.01, Paint—Tests for Chemical, Physical, and Optical Properties; Appearance,” D1544-98, 2002. Compatibility of a plasticizer with a binder and/or a solvent has been described (see, for example, Riley, H. E., “Plasticizers,” Paint Testing Manual, American Society for Testing Materials, 1972). Additionally, techniques previously described for estimating solubility for liquid and an additional coating component may be used for a plasticizer.

Various plasticizers comprise an ester of a monoalcohol and an acid (e.g., a dicarboxylic acid). In many embodiments, the monoalcohol comprises about 4 to about 13 carbons. In specific aspects, the monoalcohol comprises a butanol, an 2-ethylhexanol, an isononanol, an isooctyl, an isodecyl, or a combination thereof. Examples of an acid include an azelaic acid, a phthalic acid, a sebacic acid, a trimellitic acid, an adipic acid, or a combination thereof. Examples of such plasticizers include a di(2-ethylhexyl) azelate (“DOZ”); a di(butyl) sebacate (“DBS”); a di(2-ethylhexyl) phthalate (“DOP”); a di(isononyl) phthalate (“DINP”); a dibutyl phthalate (“DBP”); a butyl benzyl phthalate (“BBP”); a di(isooctyl) phthalate (“DIOP”); a di(idodecyl) phthalate (“DIDP”); a tris(2-ethylhexyl) trimellitate (“TOTM”); a tris(isononyl) trimellitate (“TINTM”); a di(2-ethylhexyl) adipate (“DOA”); a di(isononyl) adipate (“DINA”); or a combination thereof.

A plasticizer may be classified by a moiety, such as, for example, as an adipate (e.g., a DOA, a DINA), an azelate (e.g., a DOZ), a citrate, a chlorinated plasticizer, an epoxide, a phosphate, a sebacate (e.g., a DBS), a phthalate (e.g., a DOP, a DINP, a DIOP, a DIDP), a polyester, and/or a trimellitate (e.g., a TOTM, a TINTM). An example of a citrate plasticizer includes an acetyl tri-n-butyl citrate. Examples of an epoxide plasticizer include an epoxy modified soybean oil (“ESO”), a 2-ethylhexyl epoxytallate (“2EH tallate”), or a combination thereof. Examples of a phosphate plasticizer include an isodecyl diphenyl phosphate, a tricresyl phosphate (“TPC”), an isodecyl diphenyl phosphate, a tri-2-ethylhexyl phosphate (“TOP”), or a combination thereof. A tricresyl phosphate may function as a plastizer, confer flame resistance, confer fungi resistance, or a combination thereof, to a coating. Examples of a polyester plasticizer include an adipic acid polyester, an azelaic acid polyester, or a combination thereof. In certain aspects, a plasticizer may be selected for water resistance (e.g., hydrolysis resistance, inertness toward water) such as a bisphenoxyethylformal.

c. Water-Borne Coatings

A water-borne coating (“water reducible coating”) refers to a coating wherein a component such as a pigment, a binder, an additive, or a combination thereof are dispersed in water. Often, an additional component such as a solvent, a surfactant, an emulsifier, a wetting agent, a dispersant, or a combination thereof, promotes dispersion of a coating component. A latex coating refers to a water-borne coating wherein the binder may be dispersed in water. Typically, a binder of a latex coating comprises a high molecular weight binder. Often a latex coating (e.g., a paint, a lacquer) comprises a thermoplastic coating. Film formation occurs by loss of the liquid component, typically through evaporation, and fusion of dispersed thermoplastic binder particles. Often, a latex coating further comprises a coalescing solvent (e.g., a diethylene glycol monobutyl ether) that promotes fusion of the binder particles. In some embodiments, a film produced from a latex coating may be more porous, possesses a lower moisture resistance property, may be less compact (e.g., thicker), or a combination thereof, relative to a solvent-borne coating comprising similar non-volatile components. Specific procedures for determining the purity/properties of a latex coating, a coating component (e.g., solids content, nonvolatile content, vehicles), and/or a film have been described, for example, in “ASTM Book of Standards, Volume 06.01, Paint—Tests for Chemical, Physical, and Optical Properties; Appearance,” D4747-02 and D4827-93, 2002; “ASTM Book of Standards, Volume 06.02, Paint—Products and Applications; Protective Coatings; Pipeline Coatings,” D3793-00, 2002; and “ASTM Book of Standards, Volume 06.03, Paint—Pigments, Drying Oils, Polymers, Resins, Naval Stores, Cellulosic Esters, and Ink Vehicles,” D5097-90 D4758-92, and D4143-89, 2002.

In certain embodiments, a water-borne coating comprises a coating wherein about 50% to about 100% of a coating's liquid component comprises water. In general embodiments, the water component of a water-borne coating may function as a solvent, a thinner, a diluent, or a combination thereof. In certain embodiments, a water-borne coating may comprise an additional non-aqueous liquid component. In specific aspects, such an additional liquid component may function as a solvent, a thinner, a diluent, a plasticizer, or a combination thereof. An additional liquid component of a water-borne coating may comprise about 0% to about 49.999% of the liquid component. Examples of additional liquid components in a water-borne coating include a glycol ether, an alcohol, or a combination thereof.

In certain embodiments, an additional liquid component of a water-borne coating may be fully or partly miscible in water. Examples of a liquid that may be completely miscible in water, and visa versa, include a methanol, an ethanol, a propanol, an isopropyl alcohol, a tert-butanol, an ethylene glycol, a methyl glycol, an ethyl glycol, a propyl glycol, a butyl glycol, an ethyl diglycol, a methoxypropanol, a methyldipropylene glycol, a dioxane, a tetrahydrorfuran, an acetone, a diacetone alcohol, a dimethylformamide, a dimethyl sulfoxide, or a combination thereof. Examples of a liquid that may be partly miscible in water, by weight at about 20° C., include about 0.02% an ethylbenzene; about 0.02% a tetrachloroethylene; about 0.02% a p-xylene; about 0.035% a toluene; about 0.04% a diisobutyl ketone; about 0.1% a tricholorethylene; about 0.19% a trimethylcyclohexanol; about 0.2% a cyclohexyl acetate; about 0.3% a dibutyl ether; about 0.3% a trimethylcyclohexanone; about 0.44% a 1,1,1-tricholoroethane; about 0.53% a hexane; about 0.58% a hexanol; about 0.67% an isobutyl acetate; about 0.83% a butyl acetate; about 1.2% an isophorone; about 1.4% a nitropropane; about 1.5% a butyl glycol acetate; about 1.7% a 2-nitropropane; about 2.0% a methylene chloride; about 2.0% a methyl isobutyl ketone; about 2.3% a cyclohexanone; about 2.9% an isopropyl acetate; about 2.9% a methylbenzyl alcohol; about 3.6% a cyclohexanol; about 4.5% a nitroethane; about 4.8% a methyl tert-butyl ether; about 6.1% an ethyl acetate; about 6.9% a diethyl ether; about 7.5% a butanol; about 7.5% a butyl glycolate; about 8.4% an isobutanol; about 12.5% a 2-butanol; about 21.4% a propylene carbonate; about 23.5% an ethyl glycol acetate; about 24% a methyl acetate; and/or about 26.0% a methyl ethyl ketone. Examples of an azeotrope comprising a majority of water (BP 100° C.) include those comprising about 16.1% an isophorone (A-BP 99.5° C.); about 20% a 2-ethylhexanol (A-BP 99.1° C.); about 20% a cyclohexanol (A-BP 97.8° C.); about 20.8% a butyl glycol (A-BP 98.8° C.); and/or about 28.8% an ethyl glycol (A-BP 99.4° C.).

3. Colorants

A colorant (“colorizing agent”) comprises a composition that confers an optical property to a coating. Examples of an optical property, depending upon the application, include a reflection property, a light absorption property, a light scattering property, or a combination thereof. A colorant that increases the reflection of light may increase gloss. A colorant that increased light scattering may increase the opacity and/or confer a color to a coating and/or a film. Light scattering of a broad spectrum of wavelengths may confer a white color to a coating and/or a film. Scattering of a certain wavelength may confer a color associated with the wavelength to a coating and/or a film. Light absorption also affects opacity and/or color. Light absorption over a broad spectrum confers a black color to a coating and/or a film. Absorbance of a certain wavelength may eliminate the color associated with the wavelength from the appearance of a coating and/or a film. Examples of a colorant include a pigment, a dye, an extender, or a combination thereof. A colorant (e.g., a pigment, a dye) and procedures for determining the optical properties and physical properties (e.g., hiding power, transparency, light absorption, light scattering, tinting strength, color, particle size, particle dispersion, pigment content, color matching) of a colorant, a coating component, a coating and/or a film are described in, for example, (in “Industrial Color Testing, Fundamentals and Techniques, Second, Completely Revised Edition,” 1995; “Colorants for Non-Textile Applications,” 2000). Various colorants in the art may be used, and are often identified by their Colour Index (“CI”) number (see, for example, “Colour Index International,” 1971; and “Colour Index International,” 1997). In some cases, a common name for a colorant encompasses several related colorants, which may be differentiated by CI number.

a. Pigments

A pigment comprises a composition that is insoluble in the other component(s) of a coating, and further confers an optical properties, confers a property affecting the application of the coating (e.g., a rheological property), confers a performance property to a coating, reduces the cost of the coating, or a combination thereof. In certain embodiment, a pigment confers a performance property to a coating such as a corrosion resistance property, magnetic property, or a combination thereof. Examples of a pigment include an inorganic pigment, an organic pigment, or a combination thereof.

Pigments possess a variety of properties in addition to color that aid in the selection of a particular pigment for a specific application. Examples of such properties include a tinctorial property, an insolubility property, a corrosion resistance property, a durability property, a heat resistance property, an opacity property, a transparency property, or a combination thereof. A tinctorial property refers to the ability of a composition to produce a color, wherein a greater tinctorial strength indicating less of the composition may be used to achieve the color. An insolubility property refers to the ability of a composition to remain in a solid form upon contact with another coating component (e.g., a liquid component), even during a curing process involving chemical reactions (e.g., thermosetting, baking, irradiation). A corrosion resistance property refers to the ability of a composition to reduce the damage of a chemical (e.g., water, acid) that contacts a metal.

Pigments (e.g., extenders, titanium pigments, inorganic pigments, surface modified pigments, bismuth vanadates, cadmium pigments, cerium pigment, complex inorganic color pigments, metallic pigments, benzimidazolone pigments, diketopyrrolopyrrole pigments, dioxazine violet pigments, disazocondensation pigments, isoindoline pigments, isoindolinone pigments, perylene pigments, phthalocyanine pigments, quinacridone pigments, quinophthalone pigments, thiazine pigments, oxazine pigments, zinc sulfide pigments, zinc oxide pigments, iron oxide pigments, chromium oxide pigments, cadmium pigments, cadmium sulfide, cadmium yellow, cadmium sulfoselenide, cadmium mercury sulfide, bismuth pigments, chromate pigments, chrome yellow, molybdate red, molybdate orange, chrome orange, chrome green, fast chrome green, ultramarine pigments, iron blue pigments, black pigments, carbon black, specialty pigments, magnetic pigments, cobalt-containing iron oxide pigments, chromium dioxide pigments, metallic iron pigments, barium ferrite pigments, anti-corrosive pigments, phosphate pigments, zinc phosphate, aluminum phosphate, chromium phosphate, metal phosphates, multiphase phosphate pigments, borosilicate pigments, borate pigments, chromate pigments, molybdate pigments, lead cyanamide pigments, zinc cyanamide pigments, iron-exchange pigments, metal oxide pigments, red lead pigment, red lead, calcium plumbate, zinc ferrite pigments, calcium ferrite pigments, zinc oxide pigments, powdered metal pigments, zinc dust, lead powder, flake pigments, nacreous pigments, interference pigments, natural pearl essence pigment, basic lead carbonate pigment, bismuth oxychloride pigment, metal oxide-mica pigments, metal effect pigments, transparent pigments, transparent iron oxide pigments, transparent iron blue pigment, transparent cobalt blue pigment, transparent cobalt green pigment, transparent iron oxide, transparent zinc oxide, luminescent pigments, inorganic phosphor pigments, sulfide pigments, selenide pigments, oxysulfide pigments, oxygen dominant phosphor pigments, halide phosphor pigments, azo pigments, monoazo yellow pigments, monoazo orange pigment, disazo pigments, β-naphthol pigments, naphthol AS pigments, salt-type azo pigments, benzimidazolone pigments, disazo condensation pigments, metal complex pigments, isoindolinone pigments, isoindoline pigments, polycyclic pigments, phthalocyanine pigments, quinacrindone pigments, perylene pigments, perinone pigments, diketopyrrolo pyrrole pigments, thioindigo pigments, anthrapyrimidine pigments, flavanthrone pigments, pyranthrone pigments, anthanthrone pigments, dioxanzine pigments, triarylcarbonium pigments, quinophthalone pigments) and their chemical properties, physical properties and/or optical properties (e.g., color, tinting strength, lightening power, scattering power, hiding power, transparency, light stability, weathering resistance, heat stability, chemical fastness, interactions with a binder), in a coating component, a coating and/or a film, and techniques for determining such properties, have been described (see, for example, Solomon, D. H. and Hawthorne, D. G., “Chemistry of Pigments and Fillers,” 1983; “High Performance Pigments,” 2002; “Industrial Inorganic Pigments,” 1998; “Industrial Organic Pigments, Second, Completely Revised Edition,” 1993).

Specific standards for physical properties, chemical properties, purity, and/or procedures for testing the purity/properties of various pigments (e.g., a lead chromate, a chromium oxide, a phthalocyanine green, a phthalocyanine blue, a molybdate orange, a white zinc, a zinc oxide, a calcium carbonate, a barium sulfate, an aluminum silicate, a diatomaceous silica, a magnesium silicate, a mica, a calcium borosilicate, a zinc hydroxy phosphite, an aluminum powder, a micaceous iron oxide, a zinc phosphate, a basic lead silicochromate, a strontium chromate, an ochre, a lampblack, an orange shellac, a raw umber, a burnt umber, a raw sienna, a burnt sienna, a bone black, a carbon black, a red iron oxide, a brown iron oxide, a basic carbonate, a white lead, a white titanium dioxide, an iron blue, an ultramarine blue, a chrome yellow, a chrome orange, a hydrated yellow iron oxide, a zinc chromate yellow, a red lead, a para red toner, a toluidine red toner, a chrome oxide green, a zinc dust, a cuprous oxide, a mercuric oxide, an iron oxide, an anhydrous aluminum silicate, a black synthetic iron oxide, a gold bronze powder, an aluminum powder, a strontium chromate pigment, a basic lead silicochromate) for use in a coating are described, for example in “ASTM Book of Standards, Volume 06.03, Paint—Pigments, Drying Oils, Polymers, Resins, Naval Stores, Cellulosic Esters, and Ink Vehicles,” D280-01, D2448-85, D126-87, D305-84, D3021-01, D3256-86, D2218-67, D3280-85, D50-90, D79-86, D1199-86, D602-81, D715-86, D603-66, D718-86, D604-81, D719-91, D605-82, D717-86, D607-82, D716-86, D4288-02, D4487-90, D4462-02, D4450-85, D962-81, D5532-94, D6280-98, D1648-86, D1649-01, D85-87, D209-81, D237-57, D763-01, D765-87, D210-81, D561-82, D3722-82, D3724-01, D34-91, D81-87, D1301-91, D1394-76, D261-75, D262-81, D1135-86, D211-67, D768-01, D444-88, D3872-86, D478-02, D1208-96, D83-84, D49-83, D3926-80, D475-67, D656-87, D970-86, D3721-83, D263-75, D520-00, D521-02, D283-84, D284-88, D3720-90, D3619-77, D769-01, D476-00, D267-82, D480-88, D1845-86, D1844-86, and D279-02, 2002; and in “ASTM Book of Standards, Volume 06.01, Paint—Tests for Chemical, Physical, and Optical Properties; Appearance,” D5381-93 and D6131-97 2002.

i. Corrosion Resistance Pigments

Addition of certain pigments may improve the corrosion resistance of a coating and/or a film, such as the protection of a metal surface coated with a coating and/or a film from corrosion. Often, a primer comprises such a pigment. Examples of a corrosion resistance pigment include an aluminum flake, an aluminum triphosphate, an aluminum zinc phosphate, an ammonium chromate, a barium borosilicate, a barium chromate, a barium metaborate, a basic calcium a zinc molybdate, a basic carbonate white lead, a basic lead silicate, a basic lead silicochromate, a basic lead silicosulfate, a basic zinc molybdate, a basic zinc molybdate-phosphate, a basic zinc molybdenum phosphate, a basic zinc phosphate hydrate, a bronze flake, a calcium barium phosphosilicate, a calcium borosilicate, a calcium chromate, a calcium plumbate (CI Pigment Brown 10), a calcium strontium phosphosilicate, a calcium strontium zinc phosphosilicate, a dibasic lead phosphite, a lead chromosilicate, a lead cyanamide, a lead suboxide, a lead sulfate, a mica, a micaceous iron oxide, a red lead (CI Pigment Red 105), a steel flake, a strontium borosilicate, a strontium chromate (CI Pigment Yellow 32), a tribasic lead phophosilicate, a zinc borate, a zinc borosilicate, a zinc chromate (CI Pigment Yellow 36), a zinc dust (CI Pigment Metal 6), a zinc hydroxy phosphite, a zinc molybdate, a zinc oxide, a zinc phosphate (CI Pigment White 32), a zinc potassium chromate, a zinc silicophosphate hydrate, a zinc tetraoxylchromate, or a combination thereof.

The selection of a corrosion resistant pigment may be made based on the mechanism of corrosion resistance it confers to a coating and/or a film. Corrosion often occurs as a cathodic process wherein a metal surface acts as a cathode and passes electrons to an electron accepter moiety of a corrosive chemical, such as, for example, a hydrogen, an oxygen, or a combination thereof. Corrosion may also occur as an anodic process wherein ionized metal atoms then enter solution. A pigment such as a mica, a micaceous iron oxide, a metallic flake pigment (e.g., an aluminum, a bronze, a steel), or a combination thereof, confer corrosion resistance to a coating and/or a film by acting as a physical barrier between a metal surface and corrosive chemical(s). However, a chemically reactive pigment such as a metal flake pigment may be used in an environment at or near neutral pH (e.g., about pH 6 to about pH 8). A micaceous iron oxide may be selected for a primer, a topcoat, or a combination thereof, and may also function as a UV absorber. An aluminum flake may be selected for an industrial coating, an automotive coating, an architectural coating, a primer, or a combination thereof. An aluminum flake may additionally confer heat resistance, moisture resistance, UV resistance, or a combination thereof to a coating and/or a film. An aluminum flake may also be stearate modified for use in a topcoat. However, an aluminum flake may produce gas in a coating comprising more than about 0.15% water. A metallic zinc pigment (e.g., a zinc flake, a zinc dust) acts by functioning as an anode instead of the metal surface (e.g., a steel). However, the effectiveness of a coating's corrosion resistance fades as the zinc pigment may be used up in protective reaction(s). A metallic zinc primer may be selected for a primer, particularly in combination with an epoxy topcoat, a urethane topcoat, or a combination thereof.

A red lead and/or a basic lead silicochromate may confer an orange color, and may be selected for combination with an oil-based coating (e.g., a primer), as the pigment chemically reacts with an oil-based binder to produce a corrosion resistant lead soap in the coating and/or the film. A red lead and/or a basic lead may be selected for a primer in an industrial steel coating.

A barium metaborate pigment acts by retarding an anodic process. A barium metaborate pigment may be chemically modified by combination with a silica to reduce solubility. A zinc borate combined with a zinc phosphate, a modified barium metaborate, or a combination thereof, typically demonstrates synergistic enhancement of corrosion resistance, as well as flame retardancy.

A zinc potassium chromate may confer a yellow color as well as an anticorrosive property. A zinc tetraoxylchromate may also confer a yellow color, and may be selected for use in a two pack poly(viny butyryl) primer. A zinc oxide may be selected for an oleoresinous coating, a water-borne coating, a primer, or a combination thereof, and may be combined with a zinc chromate and/or a calcium borosilicate, and additionally may improve thermosetting cross-linking density and/or act as a UV absorber. A strontium chromate may confer a yellow color, and may be selected for an aluminum surface, an aircraft primer, or a combination thereof. A strontium chromate may be combined with a zinc chromate in a water-borne coating, though in some embodiments the total chromate content may be less from about 0.001% to about 2%. An ammonium chromate, a barium chromate and/or a calcium chromate may be selected as a corrosion inhibitor, particularly as a flash rust inhibitor.

A zinc molybdate, a zinc phosphate, a zinc hydroxy phosphite, or a combination thereof may confer a white color. These zinc pigments function by reducing an anodic process, though a zinc hydroxy phosphite may form corrosion resistant soap in an oleoresinous-coating. A basic zinc molybdate may be selected for an alkyd-coating, an epoxide-coating, an epoxy ester-coating, a polyester-coating, a solvent-borne coating, or a combination thereof. A basic zinc molybdate-phosphate may be similar to a basic zinc molybdate, though it may provide improved corrosion resistance for a rusted steel surface. A basic calcium zinc molybdate may be selected for a water-borne coating, a two-pack polyurethane coating, a two-pack epoxy coating, or a combination thereof. A combination of a basic calcium zinc molybdate and a zinc phosphate may confer an improved adhesion property to a surface comprising an iron, and may be selected for a water-borne coating and/or a solvent-borne coating. A zinc phosphate may be selected for an alkyd coating, a water-reducible coating, a coating cured by an acid and baking, or a combination thereof. A zinc phosphate may be less selected for a marine coating for salt water embodiments. A modified zinc phosphate, such as, for example, an aluminum zinc phosphate, a basic zinc phosphate hydrate, a zinc silicophosphate hydrate, a basic zinc molybdenum phosphate, or a combination thereof may confer improved corrosion resistance for a salt water embodiment. A zinc hydroxy phosphite may be selected for a solvent-borne coating.

An aluminum triphosphate typically confers a white color, acts by chelating iron ions, and may be used for a surface comprising iron. A grade I aluminum triphosphate may be modified with a zinc and a silicate, and may be selected for an alkyd-coating, an epoxy coating, a solvent-borne coating, a primer, or a combination thereof. A grade II aluminum triphosphate may be modified with a zinc and a silicate, and may be selected for a water-borne coating and/or a solvent-borne coating. A grade III aluminum triphosphate may be modified with a zinc, and may be selected for a water-borne coating and/or a solvent-borne coating.

A silicate pigment such as a barium borosilicate, a calcium borosilicate, a strontium borosilicate, a zinc borosilicate, a calcium barium phosphosilicate, a calcium strontium phosphosilicate, a calcium strontium zinc phosphosilicate, or a combination thereof, typically acts through inhibiting an anodic and/or a cathodic process, as well as forming a corrosion resistant soap in an oleoresinous-coating. A grade I and/or a grade III calcium borosilicate may be selected for a medium oil alkyd-coating, a long oil alkyd, an epoxy ester-coating, a solvent-borne coating, an architectural coating, an industrial coating, or a combination thereof, but may be less selected for a marine coating, an epoxide-coating, a water-borne coating, or a combination thereof. A calcium barium phosphosilicate grade I pigment may be selected for a solvent-borne epoxy-coating, to confer an antisettling property to a primer comprising zinc, or a combination thereof. A calcium barium phosphosilicate grade II pigment may be selected for a water-borne coating, an alkyd-coating, or a combination thereof. A calcium strontium phosphosilicate may be selected for a water-borne acrylic lacquer, a water-borne sealant, or a combination thereof. In aspects wherein a water-borne acrylic lacquer comprises a calcium strontium phosphosilicate, about a 1:1 ratio of a zinc phosphate pigment may be included. A calcium strontium zinc phosphosilicate may be selected for an alkyd-coating, an epoxide coating, a coating cured by a catalyst and baking, a water-borne coating, or a combination thereof.

ii. Camouflage Pigments

A camouflage pigment refers to a pigment typically selected to camouflage a surface (e.g., a military surface) from visual and, in specific facets, infrared detection. Examples of a camouflage pigment include an anthraquinone black, a chromium oxide green, or a combination thereof. A chromium oxide green may be selected for embodiments wherein good chemical resistance, dull color, good heat stability, good infrared reflectance, good light fastness, good opacity, good solvent resistance, low tinctorial strength, or a combination thereof, may be suitable. An anthraquinone black (CI Pigment Black 20) may be selected for good light fastness and moderate solvent resistance, and may be selected for a camouflage coating, due to its infrared absorption property.

iii. Color Property Pigments

A color property refers to the ability of a composition to confer a visual color and/or metallic appearance to a coating and/or a coated surface. A color pigment may be categorized by a common name recognized within the art, which often encompasses several specific color pigments, each identified by a CI number.

I. Black Pigments

A black pigment comprises a pigment that confers a black color to a coating. Examples of a black pigment, identified by common name with examples of specific pigments in parentheses, include an aniline black; an anthraquinone black; a carbon black; a copper carbonate; a graphite; an iron oxide; a micaceous iron oxide; a manganese dioxide; or a combination thereof.

An aniline black (e.g., a CI Pigment Black 1); may be selected for a deep black color (e.g., strong light absorption, low light scattering) and/or fastness. A coating comprising an aniline black typically comprise relatively higher concentrations of binder, and thus often possesses a matt property.

An anthraquinone black (e.g., a CI Pigment Black 20) may be selected for good light fastness and moderate solvent resistance.

A carbon black (e.g., a CI Pigment Black 6, a CI Pigment Black 7, a CI Pigment Black 8) generally possesses properties such as chemical stability, good light fastness, good solvent resistance, heat stability, or a combination thereof. A carbon black may be categorized into separate grades, based on the intensity of a black color (“jetness”). To reduce flocculation in preparing a coating comprising a carbon black pigment, such a pigment may be incrementally added to a coating during preparation, chemically modified by surface oxidation, chemically modified by an organic compound (e.g., a carboxylic acid), or a combination thereof. Additionally, a carbon black pigment may absorb certain other coating component(s) such as a metal soap drier. Typically, increasing the concentration of the susceptible component by, for example, about two-fold or more, reduces this effect. A high jet channel black pigment may be selected for use in an automotive coating wherein a high jetness may be desired. The other grades of a carbon black pigment are often selected for an architectural coating.

A graphite (e.g., a CI Pigment Black 10) may be selected for properties such as relative chemically inertness, low in color intensity, low in tinctorial strength, an anti-corrosive property, an increase in coating spreading rate, or a combination thereof.

An iron oxide (e.g., a CI Pigment Black 11) may be selected for properties such as good chemical resistance, relative inertness, good solvent resistance, limited heat resistance, low tinctorial strength, or a combination thereof. An iron oxide possesses improved floating resistance than a carbon black, particularly in combination with a titanium dioxide.

A micaceous iron oxide may be selected for properties such as relative inertness, grayish appearance, shiny appearance, function as a UV absorber, function as an anti-corrosive pigment due to resistance to oxygen and moisture passage. However, over-dispersal of a micaceous iron oxide during coating preparation may damage the pigment.

II. Brown Pigments

A brown pigment comprises a pigment that confers a brown color to a coating. Examples of a brown pigment include an azo condensation (e.g., a CI Pigment Brown 23, a CI Pigment Brown 41, a CI Pigment Brown 42); a benzimidazolone (e.g., a CI Pigment Brown 25); an iron oxide; a metal complex brown; or a combination thereof. A synthetically produced iron oxide brown (e.g., a CI Pigment Brown 6, a CI Pigment Brown 7) may be selected for embodiments wherein a rich brown color, good lightfastness, or a combination thereof, may be suitable. A metal complex brown (e.g., a CI Pigment Brown 33) may be selected for embodiments wherein high heat stability, good fastness, or a combination thereof, may be suitable. A metal complex brown may be used, for example, in a coil coating, a coating for a ceramic surface, or a combination thereof.

III. White Pigments

A white pigment comprises a pigment that confers a white color to a coating. Examples of a white pigment include an antimony oxide; a basic lead carbonate (e.g., a CI Pigment White 25); a lithopone; a titanium dioxide; a white lead; a zinc oxide; a zinc sulphide (e.g., a CI Pigment White 7); or a combination thereof.

An antimony oxide (e.g., a CI Pigment White 11) may be chemically inert, and used in a fire resistant coating. In some embodiments, an antimony oxide may be combined with a titanium dioxide, particularly in a coating with reduced chalking and/or a coating comprises a white color.

A titanium dioxide (e.g., a CI Pigment White 6) may be resistant to heat, many chemicals, and organic solvents. A titanium dioxide may be in the form of a crystal, such as an anatase crystal, a rutile crystal, or a combination thereof. A rutile may be more opaque than an anatase. An anatase has a greater ability to chalk and may be whiter in color than a rutile. In aspects wherein a coating has resuced chalking, a titanium dioxide crystal may be reacted with an inorganic oxide to enhance chalking resistance. Examples of such an inorganic oxide include an aluminum oxide, a silicon oxide, a zinc oxide, or a combination thereof.

A white lead (e.g., a CI Pigment White 1) may be chemically reactive with an acidic binder to form a strong film with elastic properties, but also chemically reacts with sulphur to become black in color. It may be less selected in certain coatings due to the toxic nature of lead.

A zinc oxide (e.g., CI Pigment White 4) confers properties such as resistance to mildew, as well as chemically reacting with an oleoresin binder in film formation to enhance resistance to abrasion, to enhance resistance to moisture, to enhance hardness, and/or reduce chalking. However, these reactions may undesirably occur during storage. In some embodiments, it may be combined with a titanium dioxide, particularly in a coating comprising an oleoresin binder when chalking may be reduced and/or the coating comprises a white color.

A zinc sulfide (e.g., a CI Pigment White 7) may be chemically inert, and confers a strong chalking property. In certain embodiments, a zinc sulfide comprises a lithopone. A lithopone (e.g., a CI Pigment White 5) comprises a mixture of a ZnS and a barium sulphate (BaSO₄), usually from about 30% to about 60% a ZnS and about 70% to about 40% a BaSO₄.

IV. Pearlescent Pigments

A pearlescent pigment comprises a pigment that confers a pearl-like appearance to a coating. Examples of a pearlescent pigment include a titanium dioxide and a ferric oxide covered mica, a bismuth oxychloride crystal, or a combination thereof.

V. Violet Pigments

A violet pigment comprises a pigment that confers a violet color to a coating. However, a violet pigment may be used in combination with a red pigment or a blue pigment to produce a color of an intermediate hue between red and blue. Additionally, a violet pigment may be combined with a titanium dioxide to balance the slight yellow color of that white pigment. An example of a violet pigment includes a dioxanine violet (e.g., a CI Pigment Violet 23; a CI Pigment Violet 37). A dioxazine violet may be selected for embodiments wherein high heat stability, good light fastness, good solvent fastness, or a combination thereof may be suitable. A CI Pigment Violet 23 (“carbazole violet”) may be transparent and/or bluer than a CI Pigment 37, and may be used in a metallic coating. A dioxazine violet may be susceptible to flocculation, loss in a powder coating, or a combination thereof, due to small particle size.

VI. Blue Pigments

A blue pigment comprises a pigment that confers a blue color to a coating. Examples of a blue pigment include a carbazol Blue; a carbazole Blue; a cobalt blue; a copper phthalocyanine; a dioxanine Blue; an indanthrone; a phthalocyanin blue; a Prussian blue; an ultramarine; or a combination thereof.

A cobalt blue (e.g., a CI Pigment Blue 36) may be selected for embodiments wherein good chemical resistance, good lightfastness, good solvent fastness, or a combination thereof, may be suitable. An indanthrone (e.g., a CI Pigment Blue 60) may be selected for embodiments wherein a redish-blue hue, good chemical resistance, good heat resistance, good solvent fastness, transparency, improved resistance to flocculation relative to a copper phthalocyanine, or a combination thereof, may be suitable.

A copper phthalocyanine (e.g., a CI Pigment Blue 15, a CI Pigment Blue 15:1, a CI Pigment Blue 15:2, a CI Pigment Blue 15:3, a CI Pigment Blue 15:4, a CI Pigment Blue 15:6, a CI Pigment Blue 16) may be selected for embodiments wherein good color strength, good tinctorial strength, good heat stability, good lightfastness, good solvent resistance, transparency, or a combination thereof, may be suitable. A CI Pigment Blue 15 may be redish in hue, but may be chemically unstable upon contact with an aromatic hydrocarbon, and converts to a greenish blue compound. ACI Pigment Blue 15:1 comprises a form of a CI Pigment Blue 15 chemically stabilized by chlorination, greener, and tinctorially weaker than a CI Pigment Blue 15. ACI Pigment Blue 15:2 comprises a modified form of a CI Pigment Blue 15 that may be resistant to flocculation. ACI Pigment Blue 15:3 may be greenish-blue, while a CI Pigment Blue 15:4 comprises a modified form of a CI Pigment Blue 15:3 that may be resistant to flocculation. A CI Pigment Blue 16 may be transparent. Examples of a coating wherein a copper phthalocyanine may be used include a metallic automotive coating. However, as described above, a copper phthalocyanine may be susceptible to flocculation due to a small primary particle size, and various modified forms are known wherein flocculation may be reduced. Examples of modifications used to reduce flocculation adding a sulfonic acid moiety; a sulfonic acid moiety and a long chain amine moiety; an aluminum benzoate; an acidic binder (e.g., a rosin); a chloromethyl moiety; or a combination thereof, to the phthalocyanine. A modified phthalocyanine may be selected for embodiments wherein color shade, dispersibility, gloss, or a combination thereof may be suitable.

A Prussian blue (e.g., a CI Pigment Blue 27) may be selected for embodiments wherein a strong color, good heat stability, good solvent fastness, or a combination thereof may be suitable. However, a Prussian blue may be chemically unstable in alkali conditions. An ultramarine (e.g., a CI Pigment Blue 29) may be selected wherein a strong color, good heat stability, good light fastness, good solvent resistance, or a combination thereof may be suitable. However, an ultramarine may be chemically unstable in acidic conditions.

VII. Green Pigments

A green pigment comprises a pigment that confers a green color to a coating. However, often a “green pigment” comprises a mixture of a yellow pigment and a blue pigment, with the properties of each component pigment generally retained. Examples of a green pigment include a chrome green; a chromium oxide green; a halogenated copper phthalocyanine; a hydrated chromium oxide; a phthalocyanine green; or a combination thereof.

A chrome green (“Brunswick green,” e.g., a CI Pigment Green 15) comprises a combination of a Prussian blue and/or a copper phthalocyanine blue and a chrome yellow. A coating comprising a chrome green may be susceptible to a floating and/or a flooding defect. A chromium oxide green (e.g., a CI Pigment Green 17) may be selected for embodiments wherein good chemical resistance, dull color, good heat stability, good infrared reflectance, good light fastness, good opacity, good solvent resistance, low tinctorial strength, or a combination thereof may be suitable. A hydrated chromium oxide (e.g., a CI Pigment Green 18) may be similar to a chromium oxide, and may be selected for embodiments wherein good light fastness, relatively brighter appearance, relatively greater transparency, relatively less heat stability, relatively less acid stability, or a combination thereof, may be suitable. A phthalocyanine green (e.g., a CI Pigment Green 7, a CI Pigment Green 36) may be selected for embodiments wherein good chemical resistance, good heat stability, good light fastness, good solvent resistance, good tinctorial strength, color transparency, or a combination thereof, may be suitable. A CI Pigment Green 7 may be selected for a bluish green color, while a CI Pigment Green 36 may be selected for a yellower-greenish color. A phthalocyanine green may be selected for an automotive coating (e.g., a metallic coating), an industrial coating, an architectural coating, a powder coating, or a combination thereof.

VIII. Yellow Pigments

In certain embodiments, a coating may comprise a yellow pigment. A “yellow pigment” comprises a pigment that confers a yellow color to a coating. Examples of a yellow pigment include an anthrapyrimidine; an arylamide yellow; a barium chromate; a benzimidazolone yellow; a bismuth vanadate (e.g., a CI Pigment Yellow 184); a cadmium sulfide yellow (e.g., a CI Pigment Yellow 37); a complex inorganic color pigment; a diarylide yellow; a disazo condensation; a flavanthrone; an isoindoline; an isoindolinone; a lead chromate; a nickel azo yellow; an organic metal complex; a quinophthalone; a yellow iron oxide; a yellow oxide; a zinc chromate; or a combination thereof.

An anthrapyrimidine pigment (e.g., a CI Pigment Yellow 108) may be selected for embodiments wherein, moderate light fastness, moderate solvent resistance, a dull color, transparency, or a combination thereof, may be suitable.

An arylamide yellow (“Hansa® yellow,” e.g., a CI Pigment Yellow 1, a CI Pigment Yellow 3, a CI Pigment Yellow 65, a CI Pigment Yellow 73, a CI Pigment Yellow 74, a CI Pigment Yellow 75, a CI Pigment Yellow 97, a CI Pigment Yellow 111) may be selected for embodiments wherein, poor heat stability, good light fastness, poor solvent resistance, moderate tinctorial strength, or a combination thereof may be suitable. A CI Pigment 1 and/or a CI Pigment 74 are mid-yellow in hue. A CI Pigment Yellow 3 may be greenish in hue. A CI Pigment Yellow 73 may be mid-yellow in hue, and resistant to recrystallization during dispersion. A CI Pigment 97 possesses improved solvent fastness than other arylamide yellow pigment(s), and has been used in a stoving enamel, an automotive coating, or a combination thereof. Other arylamide yellow pigment(s) may be used in a water-borne coating, a coating comprising a white spirit liquid component, or a combination thereof.

A benzimidiazolone yellow (e.g., a CI Pigment Yellow 120, a CI Pigment Yellow 151, a CI Pigment Yellow 154, a CI Pigment Yellow 175, a CI Pigment Yellow 181, a CI Pigment Yellow 194) may be selected for embodiments wherein, good chemical resistance, good heat stability, good light fastness, good solvent resistance, or a combination thereof, may be suitable. A benzimidiazolone with larger particle size been used in an automotive coating, a powder coating, or a combination thereof.

A cadmium sulfide yellow (e.g., a CI Pigment Yellow 37) may be selected for embodiments wherein good stability in basic pH, good heat stability, good light fastness, good opacity, good solvent fastness, or a combination thereof may be suitable. However, a cadmium yellow comprises a cadmium, which may limit suitability relative to an environmental law or regulation.

A complex inorganic color pigment (“mixed phase metal oxide,” e.g., a CI Pigment Yellow 53, a CI Pigment Yellow 119, a CI Pigment Yellow 164); may be selected for embodiments wherein, good chemical stability, good heat resistance, good light fastness, good opacity, good solvent fastness, or a combination thereof, may be suitable. However, a complex inorganic color pigment generally produces a pale color, and may be combined with an additional pigment (e.g., an organic pigment). A complex inorganic color pigment may be selected for an automotive coating, a coil coating, or a combination thereof. A bismuth vanadate may be similar to a complex inorganic pigment, but possesses improved color of green-yellow hue, poorer light fastness, and greater use in a powder coating. A bismuth vanadate may be combined with a light stabilizer.

A diarylide yellow (e.g., a CI Pigment Yellow 12, a CI Pigment Yellow 13, a CI Pigment Yellow 14, a CI Pigment Yellow 17, a CI Pigment Yellow 81, a CI Pigment Yellow 83) may be selected for embodiments wherein, good chemical resistance, poor light fastness, good solvent resistance, good tinctorial strength, or a combination thereof, may be suitable. A diarylide yellow may be not stable at a temperature of about 200° C. or greater. A CI Pigment Yellow 83 has improved light fastness than other diarylide yellow pigments, and has been used in an industrial coating, a powder coating, or a combination thereof.

A diazo condensation pigment (e.g., a CI Pigment Yellow 93, a CI Pigment Yellow 94, a CI Pigment Yellow 95, a CI Pigment Yellow 128, a CI Pigment Yellow 166) may be selected for embodiments wherein, good chemical resistance, good heat stability, good solvent resistance, good tinctorial strength, or a combination thereof, may be suitable. A diazo condensation pigment typically may be used in a plastic, though a CI Pigment Yellow 128 has been used in a coating such as an automotive coating.

A flavanthrone pigment (e.g., a CI Pigment Yellow 24) may be selected for embodiments wherein, good heat stability, moderate light fastness, a reddish yellow hue improved to an anthrapyrimidine, transparency, or a combination thereof, may be suitable.

An isoindoline yellow pigment (e.g., CI Pigment Yellow 139, a CI Pigment Yellow 185) may be selected for embodiments wherein, good chemical resistance, good heat stability, good light fastness, good solvent resistance, moderate tinctorial strength, or a combination thereof, may be suitable. An isoindolinone yellow pigment (e.g., a CI Pigment Yellow 109, a CI Pigment Yellow 110, a CI Pigment Yellow 173) typically has been used in an automotive coating and/or an architectural coating. An isoindoline yellow pigment may be selected for embodiments wherein, good light fastness, good tinctorial strength, or a combination thereof may be suitable. However, an isoindoline pigment may not be stable in a basic pH. An isoindoline yellow pigment typically has been used in an industrial coating.

A lead chromate (e.g., a CI Pigment Yellow 34) may be selected for embodiments wherein moderate heat stability, low oil absorption, good opacity, good solvent resistance, or a combination thereof may be suitable. However, a lead chromate may be susceptible to an acidic or a basic pH, and a lower light fastness so that the pigment darkens upon irradiation by light. The pH and lightfastness properties of a commercially produced lead chromate are often improved by treatment of a lead chromate with a silica, an antimony, an alumina, a metal, or a combination thereof. Additionally, a lead chromate comprises a lead and/or a chromium, which may limit suitability relative to an environmental law or regulation. A lead chromate may comprise a lead sulfate, which may be used to modify color. Examples of a lead chromate include a lemon chrome, which comprises from about 20% to about 40% a lead sulfate and may be greenish yellow in color; a middle chrome, which comprises little lead sulfate and may be reddish yellow in color; an orange chrome, which comprises no detectable lead sulfate; and a primrose chrome, which comprises from about 45% to about 55% lead chrome and may be greenish yellow in color.

An organic metal complex (e.g., a CI Pigment Yellow 129, a CI Pigment Yellow 153) may be selected for embodiments wherein good solvent resistance may be suitable. An organic metal complex may be transparent and/or dull in color.

A quinophthalone pigment (e.g., a CI Pigment Yellow 138) may be selected for embodiments wherein, good heat stability, good light fastness, good solvent resistance, a reddish yellow hue, or a combination thereof may be suitable. A quinophthalone may be either opaque or transparent. A quinophthalone pigment has been used as a substitute for a chrome as a pigment.

A yellow iron oxide (e.g., a CI Pigment Yellow 42, a CI Pigment Yellow 43) may be selected for embodiments wherein good covering power, good disperability, good resistance to chemicals, good light fastness, good solvent resistance, a yellow with greenish hue may be desired, or a combination thereof, may be suitable. A yellow iron oxide may function as a U.V. absorber. However, a yellow iron oxide may be a duller color relative to other pigment(s), and may be susceptible to temperatures of about 105° C. or greater. Additionally, a yellow iron oxide may comprise a α-crystal, a β-crystal, a γ-crystal, or a combination thereof. Overdispersion may damage the needle-shape crystal structure, which may reduce the color intensity. Additionally, a transparent yellow iron oxide may be prepared by selecting particles with minimum size, and such a pigment may be used, for example, in an automotive coating and/or a wood coating.

IX. Orange Pigments

In certain embodiments, a coating may comprise an orange pigment. An “orange pigment” comprises a pigment that confers an orange color to a coating. Examples of an orange pigment include a perinone orange; a pyrazolone orange; or a combination thereof.

A perinone orange pigment (e.g., a CI Pigment Orange 43) may be selected for embodiments wherein very good resistance to heat, good light fastness, good solvent resistance, high tinctorial strength, or a combination thereof may be suitable.

A pyrazolone orange pigment (e.g., a CI Pigment Orange 13, a CI Pigment Orange 34) may be similar to a diarylide yellow pigment, and may be selected for embodiments wherein moderate resistance to heat, poor light fastness, moderate solvent resistance, high tinctorial strength, or a combination thereof may be suitable. However, a CI Pigment Orange 34 possesses greater lightfastness relative to a CI Pigment Orange 13, and has been used in an industrial coating and/or a replacement for a chrome.

X. Red Pigments

In certain embodiments, a coating may comprise a red pigment. A “red pigment” comprises a pigment that confers a red color to a coating. Examples of a red pigment include an anthraquinone; a benzimidazolone; a BON arylamide; a cadmium red; a cadmium selenide; a chrome red; a dibromanthrone; a diketopyrrolo-pyrrole pigment (e.g., a CI Pigment Red 254, a CI Pigment Red 255, a CI Pigment Red 264, a CI Pigment Red 270, a CI Pigment Red 272); a disazo condensation pigment (e.g., a CI Pigment Red 144, a CI Pigment Red 166, a CI Pigment Red 214, a CI Pigment Red 220, a CI Pigment Red 221, a CI Pigment Red 242); a lead molybdate; a perylene; a pyranthrone; a quinacridone; a quinophthalone; a red iron oxide; a red lead; a toluidine red; a tonor pigment (e.g., a CI Pigment Red 48, a CI Pigment Red 57, a CI Pigment Red 60, a CI Pigment Red 68); a 6-naphthol red; or a combination thereof.

A lead molybdate red pigment (e.g., a CI Pigment Red 104) may be selected for embodiments wherein good resistance to heat, moderate resistance to basic pH, good opacity, excellent solvent resistance, or a combination thereof may be suitable. A molybdate red may be bright in color, and may be combined with an organic pigment to extend a color range. However, a molybdate may be easy to disperse, and overdispersion may damage this pigment. Additionally, a molybdate red comprising a lead and/or a chromium may have limited suitability relative to an environmental law or regulation.

A cadmium red pigment (e.g., a CI Pigment Red 108) may be selected for embodiments wherein excellent resistance to heat, good lightfastness, poor resistance to acidic pH, good opacity, excellent solvent resistance, or a combination thereof may be suitable. However, a cadmium red comprises a cadmium, and may have limited suitability relative to an environmental law or regulation.

A red iron oxide pigment (e.g., a CI Pigment Red 101, a CI Pigment Red 102) may be selected for embodiments wherein excellent resistance to heat, good lightfastness, poor resistance to acidic pH, good opacity, excellent solvent resistance, or a combination thereof may be suitable. However, a cadmium red comprises cadmium, and may have limited suitability relative to an environmental law or regulation.

A β-naphthol red (e.g., a CI Pigment Red 3) may be selected for embodiments wherein modest heat resistance, good lightfastness, modest solvent resistance, or a combination thereof may be suitable.

A BON arylamide (e.g., a CI Pigment Red 2, a CI Pigment Red 5, a CI Pigment Red 12, a CI Pigment Red 23, a CI Pigment Red 112, a CI Pigment Red 146, a CI Pigment Red 170) comprises various pigment(s) that generally have good lightfastness, good solvent resistance, or a combination thereof.

A tonor pigment (e.g., a CI Pigment Red 48, a CI Pigment Red 57, a CI Pigment Red 60, a CI Pigment Red 68) comprises various pigment(s) that generally have good solvent resistance, but often have poor acid resistance, poor alkali resistance, or a combination thereof.

A benzimidazolone (e.g., a CI Pigment Red 171, a CI Pigment Red 175, a CI Pigment Red 176, a CI Pigment Red 185, a CI Pigment Red 208) comprises various pigment(s) that generally have good heat stability, excellent solvent resistance, or a combination thereof.

A disazo condensation pigment (e.g., a CI Pigment Red 144, a CI Pigment Red 166, a CI Pigment Red 214, a CI Pigment Red 220, a CI Pigment Red 221, a CI Pigment Red 242) comprises various pigments that generally have excellent heat stability, good solvent resistance, or a combination thereof.

A quinacridone (e.g., a CI Pigment Red 122, a CI Pigment Red 192, a CI Pigment Red 202, a CI Pigment Red 207, a CI Pigment Red 209) comprises a various pigments that generally have bright color, excellent heat stability, excellent solvent resistance, excellent chemical resistance, good lightfastness, or a combination thereof.

A perylene (e.g., a CI Pigment Red 123, a CI Pigment Red 149, a CI Pigment Red 178, a CI Pigment Red 179, a CI Pigment Red 190, a CI Pigment Red 224) comprises a various pigment(s) that generally have excellent heat stability, excellent solvent resistance, excellent lightfastness, or a combination thereof.

An anthraquinone (e.g., a CI Pigment Red 177) has a bright color, good heat stability, good solvent resistance, good lightfastness, or a combination thereof.

A dibromanthrone (e.g., a CI Pigment Red 168) has a bright color, moderate heat stability, good solvent resistance, excellent lightfastness, or a combination thereof.

A pyranthrone (e.g., a CI Pigment Red 216, a CI Pigment Red 226) has a dull color, moderate heat stability, good solvent resistance, poor lightfastness in combination with a titanium dioxide, or a combination thereof.

A diketopyrrolo-pyrrole pigment (e.g., a CI Pigment Red 254, a CI Pigment Red 255, a CI Pigment Red 264, a CI Pigment Red 270, a CI Pigment Red 272) comprises a various pigment(s) that generally have a bright color, good opacity, excellent heat stability, excellent solvent resistance, or a combination thereof.

XI. Metallic Pigments

In certain embodiments, a coating may comprise a metallic pigment. A “metallic pigment” comprises a pigment that confers a metallic appearance to a coating, and as previously described, a corrosion resistance pigment may comprise a metallic pigment. A metallic pigment may be selected for a topcoat, particularly to confer a metallic appearance, a primer, particularly to confer a corrosion resistance property, an automotive coating, an industrial coating, or a combination thereof. A metallic flake pigment may be selected for embodiments wherein UV and/or infrared resistance may be conferred to a coating. Additionally, as some enzymes comprise a metal atom in the active site, inclusion of a metallic pigment and/or other composition comprising a metal during coating preparation, and/or addition later (e.g., a multipack coating) may stimulate a desired enzyme activity. Examples of a metallic pigment include an aluminum flake (e.g., a CI Pigment Metal 1); an aluminum non-leafing, a gold bronze flake, a zinc dust, a stainless steel flake, a nickel (e.g., a flake, a powder), or a combination thereof.

iv. Extender Pigments

An extender pigment (“inert pigment,” “extender,” “inert,” “filler”) comprises a substance that is insoluble in the other component(s) of a coating, and further confers an optical property (e.g., opacity, gloss), a rheological property, physical property, an antisettling property, or a combination thereof, to the coating and/or the film. An extender pigment may be white or near white in color, and typically are used to provide a cheap partial substitute for a more expensive white pigment (e.g., a titanium dioxide). Often an extender has a refractive index below about 1.7. In some aspects, an extenders refractive index comprises about 1.30 to about 1.70. Examples of an inorganic extender include a barium sulphate (e.g., a CI Pigment White 21, a CI Pigment White 22); a calcium carbonate (e.g., a CI Pigment White 18); a calcium sulphate; a silicate (e.g., a CI Pigment White 19, a CI Pigment White 26); a silica (e.g., a CI Pigment White 27); or a combination thereof.

A calcium carbonate (“calcite,” “whiting,” “limestone,” a CI Pigment White 18) may be chemically inert with the exception of reaction(s) with an acid. A calcium carbonate may be used in a water-borne coating and/or a solvent-borne coating. Properties specifically associated with a calcium carbonate include conferring settling resistance, sag resistance, or a combination thereof. A precipitated calcium carbonate obtained from processing of limestone, and may have improved opacity.

A kaolin (“china clay”) may be selected for a latex coating, an alkyd coating, an architectural coating, or a combination thereof. In addition to the typical properties of an extender (e.g., opacity), kaolin may confer scrub resistance to a coating.

A talc comprises a hydrated magnesium aluminum silicate, and may be soluble in water. A talc may be selected for an architectural coating (e.g., interior, exterior), a primer, a traffic marker coating, an industrial coating, or a combination thereof. A talc comprising a platy particle shape may confer chemical resistance, water resistance, improved flow property, or a combination thereof.

A silica comprises a silicon dioxide, and may be classified as crystalline silica, diatomaceous silica or synthetic silica. A crystalline silica may be produced from crushed and ground quartz, and may be selected for an architectural coating, an industrial coating, a primer, a latex coating, a powder coating, or a combination thereof. A crystalline silica may confer burnish resistance to a coating and/or a film. A diatomaceous silica (“diatomaceous earth,” “diatomite”) comprises the mineral fossil of diatoms which were single celled aquatic plants. A diatomaceous silica may be selected for an architectural coating, a latex coating, or a combination thereof. A diatomaceous silica may also function as a flattening agent. A synthetic silica may be produced from chemical reactions, and includes, for example, a precipitated silica, a fumed silica, or a combination thereof. A precipitated silica may be selected for an industrial coating, a solvent-borne coating, or a combination thereof. A precipitated silica may also function as a flattening agent. A fumed silica may be selected for an industrial coating. A fumed silica may also function as a flattening agent, a rheology modifier, or a combination thereof.

A mica comprises a hydrous silica aluminum potassium silicate, and typically comprises a plate shaped particle. A mica may be selected for an architectural coating, an exterior coating, a traffic marker coating, a primer, or a combination thereof. A mica may also confer durability, moisture resistance, corrosion resistance, heat resistance, chemical resistance, cracking resistance, sagging resistance, or a combination thereof, to a coating and/or a film.

A barium sulfate may be classified as a baryte or a blanc fixe. A baryte may be selected for an automotive coating, an industrial coating, a primer, an undercoat, or a combination thereof. A blanc fixe has good opacity for an extender, and may be selected for an automotive coating, an industrial coating, or a combination thereof.

A wollastonite comprises a calcium metasilicate, and may be selected for a latex coating. A wollasonite may also function as an alkali pH buffer. A surface modified wollasonite may be selected for an industrial coating.

A nepheline syenite comprises an anhydrous sodium potassium aluminum silicate, and may be selected for an architectural coating, a latex coating, an interior coating, an exterior coating, or a combination thereof. A nepheline syenite may function may confer cracking resistance, scrub resistance, or a combination thereof.

A sodium aluminosilicate may be selected for a latex coating, an architectural coating, or a combination thereof. A sodium aluminosilicate may also function as a flattening agent.

An alumina trihydrate may be selected for an architectural coating, a thermoplastic coating, a thermosetting coating, or a combination thereof. An alumina trihydrate may confer flame retardancy to a film.

b. Dyes

A dye comprises a composition that is soluble in the other component(s) of a coating, and further confers a color property to the coating. Many of the compounds that give a biomolecular composition (e.g., a microorganism derived particulate material) color, such as photosynthetic pigment and/or a carotenoid pigment, may be partly or fully soluble in many non-aqueous liquids described herein. A cell-based material may be added to a coating comprising such a liquid component, the material may act as a dye, as well as a pigment and/or extender, due to the dissolving of a colored compound into the liquid component.

4. Coating Additives

A coating additive comprises any material added to a coating to confer a property other than that described for a binder, a liquid component, a colorizing agent, or a combination thereof. In addition to the examples of additives described herein, any additive in the art, in light of the present disclosures, may be included in a composition.

Examples of a coating additive include a biomolecular composition (e.g., an enzyme, a peptide, a cell-based particulate material), an antifloating agent, an antiflooding agent, an antifoaming agent, an antisettling agent, an antiskinning agent, a catalyst, a corrosion inhibitor, a film-formation promoter, a leveling agent, a matting agent, a neutralizing agent, a preservative, a thickening agent, a wetting agent, or a combination thereof. The content for an individual coating additive in a coating may be about 0.000001% to about 20.0%. However, in many embodiments, the concentration of a single additive in a coating may comprise between 0.000001% and about 10.0%.

a. Preservatives

A coating may comprise a preservative to reduce and/or prevent the deterioration of a coating and/or a film by an organism such as a microorganism. A microorganism may be considered a contaminant capable damaging a film and/or a coating to the point of suitable usefulness in a given embodiment. An undesirable growth of a microorganism is generally more prevalent in a water-borne coating, as the solvent component of a solvent borne-coating usually acts as a preservative. However, a film is generally susceptible to such damage by growth of a microorganism after loss of a solvent (e.g., evaporation) during film formation. Additionally, various bacteria (e.g., Bacillus spp.) and fungi produce spores, which are cells that are relatively durable to unfavorable conditions (e.g., cold, heat, dehydration, a biocide) and may persist in a coating and/or film for months or years prior to germinating into a damaging colony of cells.

However, in certain embodiments, a biomolecular composition; particularly a microorganism based particulate material, may be used as a purposefully added coating component. A coating comprising a biomolecular composition (e.g., a cell-based particulate material) typically also comprises a preservative. The continued growth of a microorganism from a biomolecular composition often may be detrimental to a coating and/or a film, and a preservative may reduce and/or prevent such growth. A contaminating microorganism may use the biomolecular composition as a readily available source of nutrients for growth, and a preservative may reduce and/or prevent such growth. The amount of preservative added to a coating comprising a biomolecular composition may be increased relative to a preservative content of a similar coating lacking such an added biomolecular composition. In certain aspects, the amount of preservative may be increased about 1.01 to about 10-fold or more, the amount of an example of a preservative content described herein or used in the art, in light of the present disclosures.

Examples of preservatives include a biocide, which reduces and/or prevents the growth of an organism by killing the organism (e.g., a microorganism, a spore), a biostatic, which reduces and/or prevents the growth of an organism (e.g., a microorganism, a spore) but generally does not necessarily kill the organism, or a combination thereof (e.g., a combination of the effects). For example, a “fungicide” comprises a biocidal substance used to kill a specific microbial group, the fungi; while a “fungistatic” denotes a substance that prevents fungal microorganism from growing and/or reproducing, but do not result in substantial killing. Examples of a biocide include, for example, a microbiocide, a bactericide, a fungicide, an algaecide, a mildewcide, a molluskicide, a viricide, or a combination thereof. Examples of a biostatic include, for example, a microbiostatic, a bacteristatic, a fungistatic, an algaestatic, a mildewstatic, a molluskistatic, a viristatic, or a combination thereof. Examples of a bacteria commonly found to contaminate a coating and/or a film include a Pseudomonas spp., an Aerobacter spp., an Enterobacter spp., a Flavobacterium spp. (e.g., a Flavobacterium marinum), a Bacillus spp., or a combination thereof. Examples of a fungi commonly found to contaminate a coating and/or a film include an Aureobasidium pullulans, an Alternaria dianthicola, a Phoma pigmentivora, or a combination thereof. Examples of an algae commonly found to contaminate a coating and/or a film include an Oscillotoria sp., a Scytonema sp., a Protoccoccus sp., or a combination thereof. Techniques for determining microbial contamination of a coating and/or a coating component have been described (see, for example, “ASTM Book of Standards, Volume 06.01, Paint—Tests for Chemical, Physical, and Optical Properties; Appearance,” D5588-97, 2002).

In addition to the disclosures herein, a preservative and use of a preservative in a coating is known in the art, and all such materials and techniques for using a preservative in a coating may be used (see, for example, Flick, E. W. “Handbook of Paint Raw Materials, Second Edition,” 263-285 and 879-998, 1989; in “Paint and Coating Testing Manual, Fourteenth Edition of the Gardner-Sward Handbook,” (Koleske, J. V. Ed.), pp 261-267 and 654-661, 1995; in “Paint and Surface Coatings, Theory and Practice, Second Edition,” (Lambourne, R. and Strivens, T. A., Eds.), pp. 193-194, 371-382 and 543-547, 1999; Wicks, Jr., Z. W., Jones, F. N., Pappas, S. P. “Organic Coatings, Science and Technology, Volume 1: Film Formation, Components, and Appearance,” pp. 318-320, 1992; Wicks, Jr., Z. W., Jones, F. N., Pappas, S. P. “Organic Coatings, Science and Technology, Volume 2: Applications, Properties and Performance,” pp. 145, 309, 319-323 and 340-341, 1992; and in “Paints, Coatings and Solvents, Second, Completely Revised Edition,” (Stoye, D. and Freitag, W., Eds.) pp 6, 127 and 165, 1998; and in “Handbook of Coatings Additives,” pp. 177-224, 1987).

A coating, a film, a surface, or a combination thereof, may be detrimentally affected by the presence of a living organism (e.g., a microorganism). For example, a living microorganism may alter viscosity due to damage to a cellulosic viscosifier; alter a rheological property by increasing the gelling of a coating; produce a color alteration (“discoloration”) by production of a colorizing agent; produce a gas and increase foam; produce an odor; lower pH; damage a preservative; produce slime; reduce adhesion by a film; increase corrosion of a metal surface by moisture production by an organism; increase corrosion of a metal surface by film damage; damage a wooden surface by colonization (e.g., fungal colonization); or a combination thereof. These changes may lead to the coating and/or the film becoming unsuitable for use.

The quality of a liquid coating mixture may suffer markedly if a microorganism (e.g., a mold) degrades one or more of the components during storage (e.g., in-can). Since many of the coating products in use today comprise ingredients that make it susceptible or prone to microorganism (e.g., fungal) infestation and growth, it is common practice to include a preservative. Although bacterial contamination may be a contributing factor, fungi may typically be a primary cause of deterioration of a liquid paint and/or a coating. Foul odor, discoloration, thinning and clumping of the coating product, and other signs of deterioration of components render the product commercially unattractive and/or unsatisfactory for the intended purpose. If the container will be opened and closed a number of times after its initial use, in some instances over a period of several months or years, it may inevitably be inoculated with a cell such as an ambient fungus organism and/or a spore subsequent to purchase by the consumer. The growth of a microorganism may be more prevalent in a water-borne coating, as the solvent component of a solvent borne-coating usually acts as a preservative. However, a film may be susceptible to such damage by growth of a microorganism after loss of a solvent (e.g., evaporation) during film formation. Additionally, various bacteria (e.g., a Bacillus spp.) and fungi produce spore(s), which are cell(s) that are relatively durable to unfavorable condition(s) (e.g., cold, heat, dehydration, a biocide), and may persist in a coating and/or a film for month(s) and/or year(s) prior to germinating into a damaging colony of cells. To avoid spoilage, it may be desirable to ensure that the product will remain stable and usable for the foreseeable duration of storage and use by enhancing the long-term antimicrobial (e.g., antifungal) properties of the paint and/or coating with an antibiological agent (e.g., an antifungal peptide agent, an antimicrobial peptide, an antimicrobial enzyme). The in-can stability and prospective shelf life of a paint and/or coating mixture comprising an antibiological agent (e.g., a peptide agent) may be assessed using any appropriate method of the art using conventional microbiological techniques. For example, a fungus known to infect paint(s) and/or other coating(s) may be used as the challenging assay organism.

In certain embodiments, a preservative may comprise an in-can preservative, an in-film preservative, or a combination thereof. An in-can preservative comprises a composition that reduces and/or prevents the growth of a microorganism prior to film formation. Addition of an in-can preservative during a water-borne coating production typically occurs with the introduction of water to a coating composition. Typically, an in-can preservative may be added to a coating composition for function during coating preparation, storage, or a combination thereof. An in-film preservative comprises a composition that reduces or prevents the growth of a microorganism after film formation. In many embodiments, an in-film preservative comprises the same chemical as an in-can preservative, but added to a coating composition at a higher (e.g., about two-fold or more) concentration for continuing activity after film formation.

Examples of a preservative used in a coating include a metal compound (e.g., an organo-metal compound) biocide, an organic biocide, or a combination thereof. Examples of a metal compound biocide include a barium metaborate (CAS No. 13701-59-2), which may function as a fungicide and/or a bactericide; a copper(II) 8-quinolinolate (CAS No. 10380-28-6), which may function as a fungicide; a phenylmercuric acetate (CAS No. 62-38-4), a tributyltin oxide (CAS No. 56-35-9), which may be less selected for use against Gram-negative bacteria; a tributyltin benzoate (CAS No. 4342-36-3), which may function as a fungicide and a bactericide; a tributyltin salicylate (CAS No. 4342-30-7), which may function as a fungicide; a zinc pyrithione (“zinc 2-pyridinethiol-N-oxide”; CAS No. 13463-41-7), which may function as a fungicide; a zinc oxide (CAS No. 1314-13-2), which may function as a fungistatic, a fungicide and/or an algaecide; a combination of zinc-dimethyldithiocarbamate (CAS No. 137-30-4) and a zinc 2-mercaptobenzothiazole (CAS No. 155-04-4), which acts as a fungicide; a zinc pyrithione (CAS No. 13463-41-7), which may function as a fungicide; a metal soap; or a combination thereof. Examples of a metal comprised in a metal soap biocide include a copper, a mercury, a tin, a zinc, or a combination thereof. Examples of an organic acid comprised in a metal soap biocide include a butyl oxide, a laurate, a naphthenate, an octoate, a phenyl acetate, a phenyl oleate, or a combination thereof.

An example of an organic biocide that acts as an algaecide includes a 2-methylthio-4-tert-butylamino-6-cyclopropylamino-s-triazine (CAS No. 28159-98-0). Examples of an organic biocide that acts as a bactericide include a combination of a 4,4-dimethyl-oxazolidine (CAS No. 51200-87-4) and a 3,4,4-trimethyloxazolidine (CAS No. 75673-43-7); a 5-hydroxy-methyl-1-aza-3,7-dioxabicylco (3.3.0.) octane (CAS No. 59720-42-2); a 2(hydroxymethyl)-aminoethanol (CAS No. 34375-28-5); a 2-(hydroxymethyl)-amino-2-methyl-1-propanol (CAS No. 52299-20-4); a hexahydro-1,3,5-triethyl-s-triazine (CAS No. 108-74-7); a 1-(3-chloroallyl)-3,5,7-triaza-1-azonia-adamantane chloride (CAS No. 51229-78-8); a 1-methyl-3,5,7-triaza-1-azonia-adamantane chloride (CAS No. 76902-90-4); a p-chloro-m-cresol (CAS No. 59-50-7); an alkylamine hydrochloride; a 6-acetoxy-2,4-dimethyl-1,3-dioxane (CAS No. 828-00-2); a 5-chloro-2-methyl-4-isothiazolin-3-one (CAS No. 26172-55-4); a 2-methyl-4-isothiazolin-3-one (CAS No. 2682-20-4); a 1,3-bis(hydroxymethyl)-5,5-dimethylhydantoin (CAS No. 6440-58-0); a hydroxymethyl-5,5-dimethylhydantoin (CAS No. 27636-82-4); or a combination thereof. Examples of an organic biocide that acts as a fungicide include a parabens; a 2-(4-thiazolyl)benzimidazole (CAS No. 148-79-8); a N-trichloromethyl-thio-4-cyclohexene-1,2-dicarboximide (CAS No. 133-06-2); a 2-n-octyl-4-isothiazoline-3-one (CAS No. 26530-20-1); a 2,4,5,6-tetrachloro-isophthalonitrile (CAS No. 1897-45-6); a 3-iodo-2-propynyl butyl carbamate (CAS No. 55406-53-6); a N-(trichloromethyl-thio)phthalimide (CAS No. 133-07-3); a tetrachloroisophthalonitrile (CAS No. 1897-45-6); a potassium N-hydroxy-methyl-N-methyl-dithiocarbamate (CAS No. 51026-28-9); a sodium 2-pyridinethiol-1-oxide (CAS No. 15922-78-8); or a combination thereof. Examples of a parbens include a butyl parahydroxybenzoate (CAS No. 94-26-8); an ethyl parahydroxybenzoate (CAS No. 120-47-8); a methyl parahydroxybenzoate (CAS No. 99-76-3); a propyl parahydroxybenzoate (CAS No. 94-13-3); or a combination thereof. Examples of an organic biocide that acts as a bactericide and fungicide include a 2-mercaptobenzo-thiazole (CAS No. 149-30-4); a combination of a 5-chloro-2-methyl-3(2H)-isothiazoline (CAS No. 26172-55-4) and a 2-methyl-3(2H)-isothiazolone (CAS No. 2682-20-4); a combination of a 4-(2-nitrobutyl)-morpholine (CAS No. 2224-44-4) and a 4,4′-(2-ethylnitrotrimethylene dimorpholine (CAS No. 1854-23-5); a tetra-hydro-3,5-di-methyl-2H-1,3,5-thiadiazine-2-thione (CAS No. 533-74-4); a potassium dimethyldithiocarbamate (CAS No. 128-03-0); or a combination thereof. An example of an organic biocide that acts as an algaecide and fungicide includes a diiodomethyl-p-tolysulfone (CAS No. 20018-09-1). Examples of an organic biocide that acts as an algaecide, a bactericide and a fungicide include a glutaraldehyde (CAS No. 111-30-8); a methylenebis(thiocyanate) (CAS No. 6317-18-6); a 1,2-dibromo-2,4-dicyanobutane (CAS No. 35691-65-7); a 1,2-benzisothiazoline-3-one (“1,2-benzisothiazolinone”; CAS No. 2634-33-5); a 2-(thiocyanomethyl-thio)benzothiazole (CAS No. 21564-17-0); or a combination thereof. An example of an organic biocide that acts as an algaecide, a bactericide, a fungicide and a molluskicide includes a 2-(thiocyanomethyl-thio)benzothiozole (CAS No. 21564-17-0) and/or a methylene bis(thiocyanate) (CAS No. 6317-18-6).

In some embodiments, an antifungal agent (e.g., a fungicide, a fungistatic) may comprise a copper (II) 8-quinolinolate (CAS No. 10380-28-6); a zinc oxide (CAS No. 1314-13-2); a zinc-dimethyl dithiocarbamate (CAS No. 137-30-4); a 2-mercaptobenzothiazole, zinc salt (CAS No. 155-04-4); a barium metaborate (CAS No. 13701-59-2); a tributyl tin benzoate (CAS No. 4342-36-3); a bis tributyl tin salicylate (CAS No. 22330-14-9), a tributyl tin oxide (CAS No. 56-35-9); a parabens: ethyl parahydroxybenzoate (CAS No. 120-47-8), a propyl parahydroxybenzoate (CAS No. 94-13-3); a methyl parahydroxybenzoate (CAS No. 99-76-3); a butyl parahydroxybenzoate (CAS No. 94-26-8); a methylenebis(thiocyanate) (CAS No. 6317-18-6); a 1,2-benzisothiazoline-3-one (CAS No. 2634-33-5); a 2-mercaptobenzo-thiazole (CAS No. 149-30-4); a 5-chloro-2-methyl-3(2H)-isothiazolone (CAS No. 57373-19-0); a 2-methyl-3(2H)-isothiazolone (CAS No. 57373-20-3); a zinc 2-pyridinethiol-N-oxide (CAS No. 13463-41-7); a tetra-hydro-3,5-di-methyl-2H-1,3,5-thiadiazine-2-thione (CAS No. 533-74-4); a N-trichloromethyl-thio-4-cyclohexene-1,2-dicarboximide (CAS No. 133-06-2); a 2-n-octyl-4-isothiazoline-3-one (CAS No. 26530-20-1); a 2,4,5,6-tetrachloro-isophthalonitrile (CAS No. 1897-45-6); a 3-iodo-2-propynyl butylcarbamate (CAS No. 55406-53-6); a diiodomethyl-p-tolylsulfone (CAS No. 20018-09-1); a N-(trichloromethyl-thio)phthalimide (CAS No. 133-07-3); a potassium N-hydroxy-methyl-N-methyl-dithiocarbamate (CAS No. 51026-28-9); a sodium 2-pyridinethiol-1-oxide (CAS No. 15922-78-8); a 2-(thiocyanomethylthio) benzothiazole (CAS No. 21564-17-0); a 2-4(-thiazolyl)benzimidazole (CAS No. 148-79-8); or a combination thereof [see, or example, V. M. King, “Bactericides, Fungicides, and Algicides,” Ch. 29, pp. 261-267; and D. L. Campbell, “Biological Deterioration of Paint Films,” Ch. 54, pp. 654-661; both in PAINT AND COATING TESTING MANUAL, 14^{th} ed. of the Gardner-Sward Handbook, J. V. Koleske, Editor (1995), American Society for Testing and Materials, Ann Arbor, Mich.]. Additional biological products that may possess antifungal activity are described in the background discussion of U.S. Pat. Nos. 6,020,312; 5,602,097; and 5,885,782. U.S. Pat. No. 5,882,731 (Owens) describes a number of common and proprietary chemical mildewcide-comprising products that have been investigated as additives for water-based latex mixtures.

In certain embodiments an environmental law or regulation may encourage the selection of an organic biocide such as a benzisothiazolinone derivative. An example of a benzisothiazolinone derivative comprises a Busan™ 1264 (Buckman Laboratories, Inc.), a Proxel™ GXL (BIT), a Proxel™ TN (BIT/Triazine), a Proxel™ XL2 (BIT), a Proxel™ BD₂O (BIT) and a Proxel™ BZ (BIT/ZPT) (Avecia Inc.), a Preventol® VP OC 3068 (Bayer Corporation), and/or a Mergal® K10N (Troy Corp.) which comprises a 1,2-benzisothiazoline-3-one (CAS No. 2634-33-5). In the case of a Busan™ 1264, the primary use may be function as a bactericide and/or a fungicide at about 0.03% to about 0.5% in a water-borne coating, though a Busan™ may be used as a wood and/or a packaging preservative (e.g., a biocide, a mold inhibitor, a bactericide). A Proxel™ TN comprises a 1,2-benzisothiazoline-3-one (CAS No. 2634-33-5) and a hexahydro-1,3,5-tris(2-hydroxyethyl)-s-triazine (“triazine”; CAS No. 4719-04-4), a Proxel™ GXL, a Proxel™ XL2 and a Proxel™ BD₂O comprises a 1,2-benzisothiazoline-3-one (CAS No. 2634-33-5), a Proxel™ BZ comprises a 1,2-benzisothiazoline-3-one (CAS No. 2634-33-5) and a zinc pyrithione (CAS No. 13463-41-7), and are typically used in an industrial coating and/or a water-based coating as a bactericide and/or a fungicide. A Mergal® K10N comprises a 1,2-benzisothiazoline-3-one (CAS No. 2634-33-5), and may be used in a water-borne coating as a bactericide and/or a fungicide.

Often, a preservative comprises a proprietary commercial formulation and/or a compound sold under a tradename. Examples include an organic biocide under the tradename Nuosept® (International Specialty Products, “ISP”), which are typically used in a water-borne coating, often as an antimicrobial agent. Specific examples of a Nuosept® biocide include a Nuosept® 95, which comprises a mixture of bicyclic oxazolidines, and may be added to about 0.2% to about 0.3% concentration to a coating; a Nuosept® 145, which comprises an amine reaction product, and may be added to about 0.2% to about 0.3% concentration to a coating; a Nuosept® 166, which comprises a 4,4-dimethyloxazolidine (CAS No. 51200-87-4), and may be added to about 0.2% to about 0.3% concentration to a basic pH water-borne coating; or a combination thereof. A further example comprises a Nuocide® (International Specialty Products) biocide(s), which are typically used fungicide(s) and/or algaecide(s). Examples of a Nuocide® biocide comprises Nuocide® 960, which comprises about 96% tetrachlorisophthalonitrile (CAS No. 1897-45-6), and may be used at about 0.5% to about 1.2% in a water-borne and/or a solvent-borne coating as a fungicide; a Nuocide® 2010, which comprises a chlorothalonil (CAS No. 1897-45-6) and an IPBC(CAS No. 55406-53-6) at about 30%, and may be used at about 0.5% to about 2.5% in a coating as a fungicide and/or an algaecide; a Nuocide® 1051 and a Nuocide® 1071, each which comprises about 96% N-cyclopropyl-N-(1-dimethylethyl)-6-(methylthio)-1,3,5-triazine-2,4-diamine (CAS No. 28159-98-0), and may be used as an algaecide in an antifouling coating at about 1.0% to about 6.0% or a water-based coating at about 0.05% to about 0.2%, respectively; and a Nuocide® 2002, which comprises a chlorothalonil (CAS No. 1897-45-6) and a triazine compound at about 30%, and may be used at about 0.5% to about 2.5% in a coating and/or a film as a fungicide and/or an algaecide; or a combination thereof.

An additional example of a tradename biocide for a coating includes a Vancide® (R. T. Vanderbilt Company, Inc.). Examples of a Vancide® biocide include a Vancide® TH, which comprises a hexahydro-1,3,5-triethyl-s-triazine (CAS No. 108-74-7), and may be used in a water-borne coating; a Vancide® 89, which comprises a N-trichloromethylthio-4-cyclohexene-1,2-dicarboximide (CAS No. 133-06-2) and related compounds such as a captan (CAS No. 133-06-2), and may be used as a fungicide in a coating; or a combination thereof. A bactericide and/or a fungicide for a coating, particularly a water-borne coating, comprises a Dowicil™ (Dow Chemical Company). Examples of a Dowicil™ biocide include a Dowicil™ QK-20, which comprises a 2,2-dibromo-3-nitrilopropionamide (CAS No. 10222-01-2), and may be used as a bactericide at about 100 ppm to about 2000 ppm in a coating; a Dowicil™ 75, which comprises a 1-(3-chloroallyl)-3,5,7-triaza-1-azoniaadamantane chloride (CAS No. 51229-78-8), and may be used as a bactericide at about 500 ppm to about 1500 ppm in a coating; a Dowicil™ 96, which comprises a 7-ethyl bicyclooxazolidine (CAS No. 7747-35-5), and may be used as a bactericide at about 1000 ppm to about 2500 ppm in a coating; a Bioban™ CS-1135, which comprises a 4,4-dimethyloxazolidine (CAS No. 51200-87-4), and may be used as a bactericide at about 100 ppm to about 500 ppm in a coating, or a combination thereof the forgoing. An additional example of a tradename preservative (e.g., a biocide) for a coating includes a Kathon® (Rohm and Haas Company). An example of a Kathon® biocide includes a Kathon® LX, which typically comprises a 5-chloro-2-methyl-4-isothiazolin-3-one (CAS no 26172-55-4) and a 2-methyl-4-isothiazolin-3-one (CAS no 2682-20-4) at about 1.5%, and may be added from about 0.05% to about 0.15% in a coating. Examples of tradename fungicide and/or an algaecide include those described for a Fungitrol® (International Specialty Products), which typically may be used as fungicide(s), and a Biotrend® (International Specialty Products), which often is used as biocide(s); and are often formulated for a solvent-borne and/or a water-borne coating, an in-can and/or a film preservation. An example comprises a Fungitrol® 158, which comprises about 15% tributyltin benzoate (CAS No. 4342-36-3) and about 21.2% alkylamine hydrochlorides, and may be used at about 0.35% to about 0.75% in a water-borne coating for in-can and/or a film preservation. An additional example comprises a Fungitrol® 11, which comprises a N-(trichloromethylthio) phthalimide (CAS No. 133-07-3), and may be used at about 0.5% to about 1.0% as a fungicide for solvent-borne coating. A further example comprises a Fungitrol® 400, which comprises about 98% a 3-iodo-2-propynl N-butyl carbamate (“IPBC”) (Cas No. 55406-53-6), and may be used at about 0.15% to about 0.45% as a fungicide for a water-borne and/or a solvent-borne coating.

Further examples of a tradename preservative (e.g., a biocide) for a coating includes various Omadine® and/or Triadine® product(s) (Arch chemicals, Inc.), a Densil™ P, Densil™ C404 (e.g., a chlorthalonil), a Densil™ DN (BUBIT), a Densil™ DG20 and a Vantocil™ IB (Avecia Inc.), a Polyphase® 678, a Polyphase® 663, a Polyphase® CST, a Polyphase® 641, a Troysan® 680 (Troy Corp.), a Rocima® 550 (i.e., a preservative), a Rocima® 607 (i.e., a preservative), a Rozone® 2000 (i.e., a dry film fungicide), and a Skane™ M-8 (i.e., a dry film fungicide; Rohm and Haas Company) and a Myacide™ GDA, a Myacide™ GA 15, a Myacide™ Ga 26, a Myacide™ 45, a Myacide™ AS Technical, a Myacide™ AS 2, a Myacide™ AS 30, a Myacide™ AS15, a Protectol™ PE, a Daomet™ Technical and/or a Myacide™ HT Technical (BASF Corp.). A zinc Omadine® (“zinc pyrithione”; CAS No. 13463-41-7) may function as a fungicide and/or an algaecide typically used as an in-film preservative and/or an anti-fouling preservative; a sodium Omadine® (“sodium pyrithione”; CAS No. 3811-73-2) may be used as a fungicide and/or an algaecide in-film preservative; a copper Omadine® (“copper pyrithione”; CAS No. 14915-37-8) may be used as a fungicide and/or an algaecide in-film preservative and/or an anti-fouling preservative; a Triadine® 174 (“triazine,” “1,3,5-triazine-(2H,4H,6H)-triethanol”; “hexahydro-1,3,5-tris(2-hydroxyethyl)-s-triazine”; Cas No. 4719-04-4) may function as a bacteria biostatic and/or a bactericide typically used in a water-borne coating; an omacide IPBC (“Iodopropynyl-butyl carbamate”) may function as a fungicide; a Densil™ P comprises a dithio-2,2-bis(benzmethylamide) (CAS No. 2527-58-4) and may be used in an industrial coating, a water-based coating and/or a film as a fungicide and/or a bactericide; a Densil™ C404 comprises a 2,4,5,6-tetrachloroisophthalonitrile (“chlorothalonil”; CAS No. 1897-45-6) and may be used as a fungicide; a Densil™ DN and a Densil™ DG20 comprise a N-butyl-1,2-benzisothiazolin-3-one (CAS No. 4299-07-4), and each may be used as a fungicide; a Vantocil™ IB comprises a poly(hexamethylene biguanide) hydrochloride (“PHMB”; CAS No. 27083-27-8) and may function as a microbiocide; a Polyphase® 678 comprises carbendazim (CAS No. 10605-21-7) and a 3-iodo-2-propynyl butyl carbamate (CAS No. 55406-53-6), and may be used as an antimicrobial biocide for an exterior coating and/or a surface treatment; a Polyphase® 663 comprises a 3-iodo-2-propynyl butyl carbamate (CAS No. 55406-53-6), a carbendazim (CAS No. 10605-21-7) and a diuron (CAS No. 330-54-1) and may be used as a fungicide and/or an algaecide in an exterior coating; a Rocima® 550 comprises a 2-methyl-4-isothiazolin-3-one (CAS No. 2682-20-4), and may be used as a bactericide and/or a fungicide for a water-borne coating; a Rozone® 2000 comprises a 4,5-dichloro-2-N-octyl-3(2H)-isothiazolone (CAS No. 64359-81-5) and may be used as a microbiocide for a latex coating; a Skane™ M-8 comprises a 2-Octyl-4-isothiazolin-3-one (CAS No. 26530-20-1), and may be used as an in-film fungicide; a Myacide™ GDA Technical (50% Glutaraldehyde), a Myacide™ GA 15, a Myacide™ Ga 26 and a Myacide™ 45 each comprise a glutaraldehyde solution (CAS No. 111-30-8), and are typically used as an algaecide, a bactericide, and/or a fungicide; a Myacide™ AS Technical (Bronopol, solid), a Myacide™ AS 2, Myacide™ AS 30, a Myacide™ AS15 each comprise a 2-bromo-2-nitropropane-1,3-diol solution (“bronopol”; Cas No. 52-51-7) and are typically used as an algaecide; a Protectol™ PE comprises a phenoxyethanol liquid (CAS No. 122-99-6) and may be used as a microbiocide and/or a fungicide; a Dazomet™ Technical comprises a 3,5-dimethyl-2H-1,3,5-thiadiazinane-2-thione solid (“dazomet”; CAS No. 533-74-4) and may be used as a microbiocide and/or a fungicide; a Myacide™ HT Technical comprises a 1,3,5-tris-(2-hydroxyethyl)-1,3,5-hexahydrotriazine liquid (“Triazine,” CAS No. 4719-04-4) and may be used as a microbiocide and/or a fungicide. Additional examples of tradename preservatives (all from Cognis Corp., Ambler, Pa.) includes a Nopcocide® N400, which comprises a Cholorthalonil-40% solution; a Nopcocide® N-98, which comprises a Chlorothalonil-100%; a Nopcocide® P-20, which comprises an IPBC-20% solution; a Nopcocide® P-40, which comprises an IPBC-40% solution; a Nopcocide® P-100, which comprises an IPBC-100% active; or a combination thereof.

Determination of whether damage to a coating and/or a film may be due to a microorganism (e.g., a film algal defacement, a film fungal defacement), as well as the efficacy of addition of a preservative to a coating and/or a film composition in reducing microbial damage to a coating and/or a film, may be empirically determined [see, for example, Flick, E. W. “Handbook of Paint Raw Materials, Second Edition,” 263-285 and 879-998, 1989; in “Paint and Coating Testing Manual, Fourteenth Edition of the Gardner-Sward Handbook,” (Koleske, J. V. Ed.), pp 261-267 and 654-661, 1995; in “Paint and Surface Coatings, Theory and Practice, Second Edition,” (Lambourne, R. and Strivens, T. A., Eds.), pp. 193-194, 371-382 and 543-547, 1999; Wicks, Jr., Z. W., Jones, F. N., Pappas, S. P. “Organic Coatings, Science and Technology, Volume 1: Film Formation, Components, and Appearance,” pp. 318-320, 1992; Wicks, Jr., Z. W., Jones, F. N., Pappas, S. P. “Organic Coatings, Science and Technology, Volume 2: Applications, Properties and Performance,” pp. 145, 309, 319-323 and 340-341, 1992; in “Paints, Coatings and Solvents, Second, Completely Revised Edition,” (Stoye, D. and Freitag, W., Eds.) pp 6, 127 and 165, 1998; In “Waterborne Coatings and Additives,” 202-216, 1995; in “Handbook of Coatings Additives,” pp. 177-224, 1987; and in “PCI Paints & Coatings Industry,” pp. 56, 58, 60, 62, 64, 66-68, 70, 72 and 74, July 2003]. In conducting such tests, microorganisms such as, for example, Gram-negative Eubacteria including Alcaligenes faecalis (ATCC No. 8750), Pseudomonas aeruginosa (ATCC Nos. 10145 and 15442), Pseudomonas fluorescens (ATCC No. 13525), Enterobacter aerogenes (ATCC No. 13048), Escherichia coli (ATCC No. 11229), Proteus vulgaris (ATCC No. 8427), Oscillatoria sp. (ATCC No. 29135), and Calothrix sp. (ATCC No. 27914); Gram-positive Eubacteria including Bacillus subtilis (ATCC No. 27328), Brevibacterium ammoniagenes (ATCC No. 6871), and Staphylococcus aureus (ATCC No. 6538); filamentous fungi including Aspergillus oryzae (ATCC No. 10196), Aspergillus flavus (ATCC No. 9643), Aspergillus niger (ATCC Nos. 9642 and 6275), Aureobasidium pullulans (ATCC No. 9348), Penicillium sp. (ATCC No. 12667), Penicillium citrinum (ATCC No. 9849), Penicillium funiculosum (ATCC No. 9644), Cladosporium cladosporoides (ATCC No. 16022), Trichoderma viride (ATCC No. 9645), Ulocladium atrum (ATCC No. 52426), Alternaria alternate (ATCC No. 52170), and Stachybotrys chartarum (ATCC No. 16026); yeast including Candida albicans (ATCC No. 11651); and Protista including Chlorella sp. (ATCC No. 7516), Chlorella vulgaris (ATCC No. 11468), Chlorella pyrenoidosa (UTEX No. 1230), Chlorococcum oleofaciens (UTEX No. 105), Ulothrix acuminata (UTEX No. 739), Ulothrix gigas (ATCC No. 30443), Scenedesmus quadricauda (ATCC No. 11460), Trentepohlia aurea (UTEX No. 429), and Trentepohlia odorata (CCAP No. 483/4); have been used as positive control contaminants of a coating.

b. Wetting Additives and Dispersants

One or more types of a particulate matter (e.g., a pigment, a cell-based particulate material) may be incorporated into a coating composition. Physical force and/or chemical additives are used to promote dispersion of a particulate matter in a coating composition, for purposes such as coating homogeneity and ease of application. Depending upon whether such an additive may be admixed earlier or latter in a coating composition, such an additive may be known as a wetting agent or a dispersant, respectively, though such an additive may have dual classification. A wetting agent and/or a dispersant often may be used to reduce the particulate matter grinding time during coating preparation, improve wetting of a particulate matter, improve dispersion of a particulate matter, improve gloss, improve leveling, reduce flooding, reduce floating, reduce viscosity, reduce thixotropy, or a combination thereof.

In certain embodiments, a biomolecular composition (e.g., a cell-based particulate material) may be used as a wetting additive and/or a dispersant. Though this use may be counter-intuitive, in embodiments such as a cell-based particulate material may promote the separation of particulate material (e.g., a pigment, an additional preparation of a cell-based particulate material) by acting as a physical barrier between particles of a particulate material. In embodiments wherein the cell-based particulate material may be used as a wetting additive and/or a dispersant, it may, of course, be combined with a traditional wetting additive and/or a dispersant, examples of which are described below.

i. Wetting Additives

Preparation of a coating comprising a particulate material often comprises a step wherein the particulate material may be dispersed in an additional coating component. An example of this type of dispersion step may be the dispersion of a pigment into a combination of a liquid component and a binder to form a material known as a millbase. A wetting additive (“wetting agent”) comprises a composition added to promote dispersion of a particulate material during coating preparation.

In certain embodiments, a wetting agent comprises a molecule comprising a polar region and a nonpolar region. An example comprises an ethylene oxide molecule comprising a hydrophobic moiety. Such a wetting agent may act by reducing interfacial tension between a liquid component and particulate matter. In specific aspects, a wetting agent comprises a surfactant. Examples of such a wetting agent include a pine oil, which may be added at about 1% to about 5% of the total coating liquid component. Other examples of a wetting agent include a metal soap (e.g., a calcium octoate, a zinc octoate, an aluminum stearate, a zinc stearate). An additional example of a wetting agent comprises a bis(2-ethylhexyl)sulfosuccinate (“Aerosol OT”) (Cas No. 577-11-7); an (octylphenoxy)polyethoxyethanol octylphenyl-polyethylene glycol (“Igepal-630”) (Cas no. 9036-19-5); a nonyl phenoxy poly(ethylene oxy)ethanol (“Tergitol NP-14”) (Cas No. 9016-45-9); an ethylene glycol octyl phenyl ether (“Triton X-100”) (CAS No. 9002-93-1); or a combination thereof.

Often a wetting agent and/or a dispersant comprises a proprietary formulation and/or commonly available under a trade name. Examples of a wetting agent and/or a dispersant include an Anti-Terra® and/or a Disperbyk® (BYK-Chemie GmbH), and/or an EnviroGem® and/or a Surfynol® (Air Products and Chemicals, Inc.). An example comprises an Anti-Terra®-U, which comprises about a 50% solution of an unsaturated polyamine amide salt and a lower molecular weight acid, dissolved in a xylene and an isobutanol, and may be selected for used in a solvent-borne coating. An anti-Terra®-U may be added from about 1% to about 2% to an inorganic pigment, about 1% to about 5% to an organic pigment, about 0.5% to about 1.0% to titanium dioxide, and/or about 30% to about 50% to a bentonite, respectively. An example of a Disperbyk® comprises a Disperbyk®, which comprises a polycarboxylic acid polymer alkylolammonium salt and water, and may be added to about 0.3% to about 1.5%, respectively, to the solvent-borne and/or the water-borne coating composition. A further example comprises a Disperbyk®-101, which comprises about a 52% solution of a long chain polyamine amide salt and a polar acidic ester, dissolved in a mineral spirit and butylglycol, and may be used in a solvent-borne coating. The ranges for addition to particulate material for a Disperbyk®-101 may be similar to an Anti-Terra®-U. An additional example comprises a Disperbyk®-108, which comprises over about 97% of a hydroxyfunctional carboxylic acid ester that includes moiety(s) with pigment affinity, and may be added from about 3% to about 5% to an inorganic pigment, and/or about 5% to about 8% to an organic pigment, respectively. However, a Disperbyk®-108 may be added at about 0.8% to about 1.5% to a titanium dioxide, and/or about 8% to about 10% to a carbon black, respectively, and may be used for coatings lacking a non-aqueous solvent. A supplemental example comprises an EnviroGem® AD01, which comprises a non-ionic wetting agent with a defoaming property, and may be added to about 0.1% to about 2%, to a water-borne coating composition. An additional example comprises a Surfynol® TG (Air Products and Chemicals, Inc.), which comprises a non-ionic wetting agent, and may be added to about 0.5% to about 5%, to a water-borne coating composition. A further example comprises a Surfynol® 104 (Air Products and Chemicals, Inc.), which comprises a non-ionic wetting agent, a dispersant, and a defoamer, and may be added to about 0.05% to about 3%, to a water-borne coating.

ii. Dispersants

Maintenance of the dispersal of a particulate matter comprised within a coating composition may be promoted by the addition of a dispersant. A dispersant (“dispersing additive,” “deflocculant,” “antisettling agent”) comprises a composition added to promote continuing dispersal of a particulate matter. In specific aspects, a dispersant may be added to a coating composition to reduce or prevent flocculation. Flocculation refers to the process wherein a plurality of primary particles that have been previously dispersed form an agglomerate. In other aspects, a dispersant may be added to a coating composition to prevent sedimentation of a particulate matter. Standard procedures to determining the degree of settling by a particulate matter in a coating (e.g., paint) are described, for example, in “ASTM Book of Standards, Volume 06.02, Paint—Products and Applications; Protective Coatings; Pipeline Coatings,” D869-85, 2002.

Often a dispersant comprises a compound comprising a phosphate, such as, for example, a tetra-potassium pyrophosphate (“TKPP”); CAS No. 7320-34-5). Examples of a tradename/proprietary phosphate compound are those known as a Strodex™ (Dexter Chemical L.L.C.), including a Strodex™ PK-90, a Strodex™ PK-OVOC, and/or a Strodex™ MOK-70, which comprise a phosphate ester surfactant.

In some aspects, a dispersant may comprise a particulate material. Examples include a Winnofil® SPT Premium, a Winnofil® S, Winnofil® SPM, and/or a Winnofil® SPT (Solvay Advanced Functional Minerals), which comprise about 97.4% calcium carbonate (CAS No. 471-34-1) coated with about 2.6% fatty acid (CAS No. 64755-01-7) and generally used at about 2% to about 3%.

A dispersant may comprise a modified montmorillonite. Examples include a Bentone® (Elementis Specialties, Inc). A Bentone® 34 (Elementis Specialties, Inc) comprises a tetraallyl ammonium bentonite, and may be prepared with about 33% or more polar solvent prior to addition to a coating composition. A M-P-A® 14 (Elementis Specialties, Inc.) comprises a montmorillonite clay modified by and organic chemical, and may be prepared with about 33% or more polar solvent prior to addition to a solvent-borne coating composition. A Bentone® SD-1 (Elementis Specialties, Inc.) comprises a montmorillonite clay modified by an organic chemical, and typically added from about 0.2% to about 2%, by weight to a solvent-borne coating composition, particularly those comprising an aliphatic liquid component.

A further example of a dispersant comprises a castor wax formulation under the trade names Crayvallac® SF, Crayvallac® MT, and/or Crayvallac® AntiSettle CVP (Cray Valley Limited), each of which are typically added from about 0.2% to about 1.5%, as a dispersant, a thixotropy additive, an anti-sagging agent, or a combination thereof. A Crayvallac® AntiSettle CVP comprises a caster wax (“hydrogenated caster oil”), and may be suitable for a solvent free epoxy-coating and a mineral spirit liquid component. A Crayvallac® SF and/or a Crayvallac® MT each comprise an amide modified caster wax, and may be used in an epoxy-coating, an acrylic-coating, a chlorinated rubber-coating, or a combination thereof. A Crayvallac® SF and/or a Crayvallac® MT may be used with a liquid component comprising an aromatic hydrocarbon, an alcohol, a glycol ether, or a combination thereof with a Crayvallac® MT being also may be used with a mineral spirit.

c. Buffers

In certain embodiments, a material formulation's (e.g., a coating) pH may be maintained within a certain range. The pH may range from about 0 to about 14. A coating may be acidic, which refers to a pH between about 0 and about 7, or basic, which refers to a pH between about 7 and about 14. A neutral pH refers to a pH about 7.0, and a coating may have a neutral pH, or a pH that is near neutral, which refers to a pH between about 6.5 and about 7.5. A buffer may be added to maintain a coating's pH in a desired range, such as, for example, acidic, basic, neutral, and/or near neutral.

In some embodiments, the pH buffer may be selected to help maintain the pH of a material formulation (e.g., a coating) to promote the activity of a biomolecular composition, such as an enzyme's activity. For example, in certain aspects, a basic pH may improve the function of an enzyme, such as, for example, a lipolytic enzyme and/or OPH that functions better in basic pH range. For example, an acid released by a lipolytic enzyme's activity may detrimentally alter the local pH relative to optimum conditions for activity, and a buffer may reduce this effect. Alternatively, the buffer may be selected for biomolecular compositions that function at neutral and/or basic pH, or to effect the function of other components of a material formulation, such as, for example, the curing process. Examples of a buffers includes a bicarbonate (e.g., an ammonium bicarbonate), a monobasic phosphate buffer, a dibasic phosphate buffer, a Trizma base, a 5 zwitterionic buffer, a triethanolamine, or a combination thereof. In particular facets, a buffer such as a bicarbonate, may provide a ligand and/or co-substrate (e.g., water) on activator (e.g., carbon dioxide) to an enzyme to promote an enzymatic reaction. In particular facets, a buffer may comprise about 0.000001 M to about 2.0 M, in a material formulation.

d. Rheology Modifiers

A rheology modifier (“rheology control agent,” “rheology additive,” “thickener and rheology modifier,” “TRM,” “rheological and viscosity control agent,” “viscosifier,” “viscosity control agent,” “thickener”) comprises a composition that alters (e.g., increases, decreases, maintains) a rheological property of a coating. A thickener (“thickening agent”) increases and/or maintains viscosity. A rheological property refers to a property of flow and/or deformation. Examples of a rheological property include viscosity, brushability, leveling, sagging, or a combination thereof. Viscosity comprises a measure of a fluid's resistance to flow (e.g., a shear force). Brushability refers to the ease a coating may be applied using an applicator (e.g., a brush). Leveling refers to the ability of a coating to flow into and fill uneven areas of coating thickness (e.g., brush marks) after application to a surface and before sufficient film formation to end such flow. Sagging refers to the gravitationally induced downward flow of a coating after application to a surface and before sufficient film formation to end such flow. A cell-based particulate material may be added to a coating as a rheology modifier. In embodiments wherein the cell-based particulate material may be used as a rheology modifier, it may, of course, be combined with a traditional rheology modifier, examples of which are described below.

A rheology modifier that alters viscosity (e.g., increases, decreases, maintains) may be known as a “viscosifier.” During preparation, the viscosity of a coating (“medium-shear viscosity,” “mid-shear viscosity,” “coating consistency”) may be measured to verify a viscosity that may be suitable for a coating during storage, application, etc. The typical range of shear force for measuring mid-shear viscosity comprises between about 10 s⁻¹ to about 10³ s⁻¹. In many embodiments, particularly for an architectural coating, a medium shear viscosity may be between about 60Ku to about 140Ku. During application (“high-shear”), a coating may be subjected to a shear force of about 10³s⁻¹ to about 10⁴ s⁻¹, by techniques such as brush application, and a shear force up to or greater than about 10⁶s⁻¹ by techniques including, for example, blade application, high-speed roller application, spray application, or a combination thereof. A coating may be formulated to possess a viscosity upon the shear force of application (“high-shear viscosity”) that promotes the ease of application. An example of a high shear viscosity during application comprises between about 0.5 P (“50 mPa s”) to about 2.5 P (“250 mPa s”). In certain aspects, a coating may possess a viscosity greater or lower than this range, however, such a viscosity may make the coating more difficult to apply using the above application techniques. Post-preparation and/or post-application, a coating may be subjected to a shear force of about 10 s⁻¹ to about 10⁻³ s⁻¹, may be produced, for example, by forces such as gravity, capillary pressure, or a combination thereof. In embodiments wherein a coating's viscosity (“low-shear viscosity”) may be too high at these levels of shear force (“low-shear”), leveling during and/or after application may be undesirably low. In embodiments wherein a viscosity may be too low at these levels of shear force, a coating may suffer in-can settling, sagging during or after application, or a combination thereof. In some embodiments, viscosity of a coating post-preparation and/or application may be between about 100 P (“10 Pa s”) to about 1000 P (“100 Pa s”). In other aspects, the coating has a viscosity of about 100 P to about 1000 P, upon a surface immediately after application. In some embodiments, the viscosity of the coating varies during preparation (“mixing”), during storage (e.g., in a container), during application, and/or upon a surface. The medium-shear viscosity (“coating consistency”) refers to the viscosity of a coating during preparation, and in many embodiments may be between about 60 Ku to about 140 Ku. Specific examples of medium-shear viscosity intermediate ranges and combinations thereof include about 70 Ku to about 110 Ku; about 80 Ku to about 100 Ku; about 90 Ku to about 95 Ku; about 72 Ku to 95 Ku; etc. During storage and upon a surface, a coating may be subject to lower shear forces (e.g., gravity), and a coating may possess a viscosity and other rheological propertie(s) (e.g., leveling, sag, syneresis, settling) to retain suitable dispersion of coating components during storage and form a uniform layer upon a surface. In many embodiments, the low-shear viscosity (e.g., the viscosity prior to application, viscosity upon a surface immediately after application) of a coating may be between about 100 P to about 3000 P. Specific examples of low-shear viscosity intermediate ranges and combinations thereof include about 100 P to about 2500 P; about 100 P to about 2000 P; about 100 P to about 1500 P; about 100 P to about 1000 P; about 125 P to about 3000 P; about 150 P to about 3000 P; about 175 P to about 3000 P; about 200 P to about 3000 P; about 225 P to about 3000 P; about 250 P to about 3000 P; about 275 P to about 3000 P; about 300 P to about 3000 P; about 125 P to about 2500 P; about 150 P to about 2000 P; about 175 P to about 1500 P; about 200 P to about 1000 P; and/or about 250 P to about 1000 P; about etc., respectively. The high-shear viscosity (“application viscosity”) refers to the viscosity of a coating during application, and may be less than the low-shear viscosity to allow ease of application. In particular aspects, the coating has a high-shear viscosity of about 0.5 P to about 2.5 P. Specific examples of high-shear viscosity intermediate ranges and combinations thereof include about 0.5 P to about 2.0 P; about 0.5 P to about 1.5 P; about 0.5 P to about 1.0 P; about 0.5 P to about 0.75 P; about 0.6 P to about 2.5 P; about 0.75 P to about 2.5 P; about 1.0 P to about 2.5 P; about 1.5 P to about 2.5 P; about 2.0 P to about 2.5 P; about 0.75 P to about 2.0 P; and/or about 1.0 P to about 2.0 P; etc., respectively. Of course, the viscosity of a coating changes post-application in embodiments wherein film formation occurs; however, the post-application viscosity refers to the viscosity prior to completion of film formation, and may be determined immediately post-application (e.g., within seconds, within minutes) as appropriate to the coating, using technique in the art. In certain aspects, a coating may possess a viscosity greater or lower than this range, however, such a viscosity may make the coating more prone to sagging and/or settling defects. Techniques for measuring viscosity (e.g., low-shear viscosity, medium-shear viscosity, high-shear viscosity) are known in the art [see, for example, “ASTM Book of Standards, Volume 06.01, Paint—Tests for Chemical, Physical, and Optical Properties; Appearance,” D562-01, D2196-99, D4287-00, 2002; and in “Paint and Coating Testing Manual, Fourteenth Edition of the Gardner-Sward Handbook,” (Koleske, J. V. Ed.), 1995].

A rheology modifier may be added to alter and/or maintain a rheology property within a desired range post-formulation, during application, post-application, or a combination thereof. In specific embodiments, a rheology modifier alters viscosity at or above 10³ s⁻¹ and/or at or below 10 s⁻¹. Viscosity, including non-Newtonian (e.g., shear-thinning) viscosity for a coating and/or a coating component(s) (e.g., a binder, a binder solution, a vehicle) upon formulation with or without a viscosity modifier may be empirically determined, particularly for shear rates comparable to application techniques (e.g., blade, brush, roller, spray) by standard techniques such as in “ASTM Book of Standards, Volume 06.01, Paint—Tests for Chemical, Physical, and Optical Properties; Appearance,” D562-01, D2196-99, D4287-00, D4212-99, D1200-94, D5125-97, and D5478-98, 2002; “ASTM Book of Standards, Volume 06.02, Paint—Products and Applications; Protective Coatings; Pipeline Coatings,” D4958-97, 2002; and “ASTM Book of Standards, Volume 06.03, Paint—Pigments, Drying Oils, Polymers, Resins, Naval Stores, Cellulosic Esters, and Ink Vehicles,” D1545-98, D1725-62, D6606-00 and D6267-98, 2002. Additionally, other rheological properties may be determined to aid formulation of a coating using techniques in the art. For example, brush drag, which refers to the resistance during coating (e.g., a latex) application using a brush, may be determined by standard techniques, such as, for example, in “ASTM Book of Standards, Volume 06.02, Paint—Products and Applications; Protective Coatings; Pipeline Coatings,” D4040-99, 2002. In an additional example, leveling and sagging may be empirically determined for a coating by standard techniques such as in “ASTM Book of Standards, Volume 06.02, Paint—Products and Applications; Protective Coatings; Pipeline Coatings,” D4062-99 and D4400-99, 2002.

The addition of a coating component to a coating composition typically alters a rheological property, and many coating components have multiple classifications to include function as a rheology modifier. Examples of coating components more commonly added for function as a rheology modifier include an inorganic rheology modifier, an organometallic rheology modifier, an organic rheology modifier, or a combination thereof. An example of an inorganic rheology modifier includes a silicate such as a montmorillonite silicate. An example of a montomorillonite silicate includes an aluminum silicate, a bentonite, a magnesium silicate, or a combination thereof. A silicate rheology modifier typically confers an improved washfastness property, an improved abrasion resistance property, or a combination thereof, to a coating relative to an organic rheology modifier. An example of an organic rheology modifier includes a cellulose ether, a hydrogenated oil, a polyacrylate, a polyvinylpyrrolidone, a urethane, or a combination thereof. An organic rheology modifier of a polymeric nature (e.g., a cellulose ether, a urethane, a polyacrylate, etc.) are sometimes used as an associative thickener, and may be used for a latex coating. An organic rheology modifier typically confers a greater water retention capacity property (“open time”) to a coating relative to a silicate rheology modifier. A common example of a cellulose ether comprises a methyl cellulose, a hydroxyethyl cellulose, or a combination thereof. An example of a hydroxyethyl cellulose includes a Natrosol® (Hercules Incorporated); a Cellosize™ (Dow Chemical Company); or a combination thereof. An example of a hydrogenated oil includes a hydrogenated castor oil. An example of a urethane rheology modifier (“associative thickener”) includes a hydrophobically modified ethylene oxide urethane (“HEUR”), which comprises a polyethylene glycol block covalently linked by urethane, and has both a hydrophilic and a hydrophobic region capable of use in an aqueous environment. An example of a HEUR includes a block of polyethylene oxide linked by a urethane and modified with a nonyl phenol hydrophobe (Rohm and Haas Company). Often a urethane rheology modifier confers an improved leveling property over another type of an organic rheology modifier. An example of an organometallic rheology modifier includes a titanium chelate, a zirconium chelate, or a combination thereof.

In addition to the disclosures herein, a rheology modifier and use of a rheology modifier in a coating is known in the art, and such compositions and techniques may be included (see, for example, Flick, E. W. “Handbook of Paint Raw Materials, Second Edition,” 808-843 and 879-998, 1989; in “Paint and Coating Testing Manual, Fourteenth Edition of the Gardner-Sward Handbook,” (Koleske, J. V. Ed.), pp 268-285 and 348-349, 1995; in “Paint and Surface Coatings, Theory and Practice, Second Edition,” (Lambourne, R. and Strivens, T. A., Eds.), pp. 73, 218, 227, 352, 558-559 and 718, 1999; Wicks, Jr., Z. W., Jones, F. N., Pappas, S. P. “Organic Coatings, Science and Technology, Volume 2: Applications, Properties and Performance,” pp. 42, 215, 293, 315, 320 and 323-328, 1992; and in “Paints, Coatings and Solvents, Second, Completely Revised Edition,” (Stoye, D. and Freitag, W., Eds.) pp 6, 128 and 166-167, 1998.

e. Defoamers

A coating sometimes comprises a gas capable of forming a bubble (“foam”) that may undesirably alter a physical and/or an aesthetic property. Gas incorporation into a coating composition may be a side effect of coating preparation processes, and a particular bane of a latex coating. Often, a wetting agent and/or a dispersant used in a coating may promote creation or retention of foam voids as a side effect. Additionally, cells (e.g., microorganisms) may produce gas, and in certain embodiments, a coating comprising a cell-based particulate material may also comprise a defoamer. A defoamer (“antifoaming agent,” “antifoaming additive”) comprises a composition that releases a gas (e.g., air) and/or reduces foaming in a coating during production, application, film formation, or a combination thereof. A defoamer often acts by lowering the surface tension around a bubble, allowing merging of a bubble with a second bubble, which produces a larger and less stable bubble that collapses. In certain coating compositions, a cell-based particulate material may act as a defoamer by destabilizing a bubble in a coating. In embodiments wherein the cell-based particulate material may be used as a defoamer, it may, of course, be combined with a traditional defoamer, examples of which are described below.

Examples of a defoamer include an oil (e.g., a mineral oil, a silicon oil), a fatty acid ester, a dibutyl phosphate, a metallic soap, a siloxane, a wax, an alcohol comprising between six to ten carbons, or a combination thereof. An example of an oil defoamer comprises a pine oil. In some aspects, an antifoaming agent may be combined with an emulsifier, a hydrophobic silica, or a combination thereof. Examples of a tradename defoamer comprises a TEGO® Foamex 8050 (Goldschmidt Chemical Corp.), which comprises a polyether siloxane copolymer and a fumed silica, and typically may be used at about 0.1% to about 0.5%, during coating preparation; and a BYK®-31 (BYK-Chemie), which comprises a paraffin mineral oil and a hydrophobic compound, and typically may be used at about 0.1% to about 0.5%, in a coating.

f. Catalysts

A catalyst comprises an additive that promotes film formation by catalyzing a cross-linking reaction in a thermosetting coating. Examples of a catalyst include a drier, an acid and/or a base, and the selection of the type of catalyst may be specific to the chemistry of the film formation reaction.

i. Driers

A drier (“siccative”) catalyzes an oxidative film formation reaction, such as those that occur in an oil-based coating. In addition to the disclosures herein, a drier and use of a drier in a coating may be known in the art, and such materials and techniques for using a drier in a coating may be used (see, for example, Flick, E. W. “Handbook of Paint Raw Materials, Second Edition,” pp. 73-93 and 879-998, 1989; in “Paint and Coating Testing Manual, Fourteenth Edition of the Gardner-Sward Handbook,” (Koleske, J. V. Ed.), pp 30-35, 1995; in “Paint and Surface Coatings, Theory and Practice, Second Edition,” (Lambourne, R. and Strivens, T. A., Eds.), pp. 190-192, 1999; Wicks, Jr., Z. W., Jones, F. N., Pappas, S. P. “Organic Coatings, Science and Technology, Volume 1: Film Formation, Components, and Appearance,” pp. 138, 317-318, 1992; Wicks, Jr., Z. W., Jones, F. N., Pappas, S. P. “Organic Coatings, Science and Technology, Volume 2: Applications, Properties and Performance” pp. 138, 197-198, 330, 344, 1992; and in “Paints, Coatings and Solvents, Second, Completely Revised Edition,” (Stoye, D. and Freitag, W., Eds.) pp. 11, 48, 165, 1998.

A drier may comprise a metal drier, an alternative drier, a feeder drier, or a combination thereof. Usually a drier comprising a metal (“a metal drier”) catalyzes the oxidative reaction. Examples of a metal typically used in a drier includes an aluminum, a barium, a bismuth, a calcium, a cerium, a cobalt, an iron, a lanthanum, a lead, a manganese, a neodymium, a potassium, a vanadium, a zinc, a zirconium, or a combination thereof. Examples of types of a metal drier include an inorganic metal salt, a metal-organic acid salt (“soap”), or a combination thereof. A “salt” comprises the composition formed between the anion of an acid and the cation of a base. Typically, the acid and the base of a salt interact by an ionic bond. Examples of an organic acid used in such a soap include a monocarboxylic acid (e.g., a fatty acid) of about 7 to about 22 carbon atoms. Examples of such a monocarboxylic acid include a linoleate, a naphthenate, a neodecanoate, an octoate, a rosin, a synthetic acid, a tallate, or a combination thereof. Examples of a drier comprising a synthetic acid include those under the tradenames Troymax™ (Troy Corporation). Though many driers are water insoluble, a water dispersible drier may be prepared by combining a surfactant with a naphthenate drier and/or a synthetic acid drier. However, a water dispersible driers are typically obtained under a tradename such as, for example, a Troykyd® Calcium WD, a Troykyd® Cobalt WD, a Troykyd® Manganese WD a Troykyd® Zirconium WD (Troy Corporation). Additionally, a potassium soap, a lithium soap, or a combination thereof, has limited aqueous solubility.

A primary drier (“surface drier,” “active drier,” “top drier”) acts at the coating-external environment interface. A secondary drier (“auxiliary drier,” through drier”) acts throughout the coating. Examples of a primary drier include a metal drier comprising a cobalt, a manganese, a vanadium, or a combination thereof. Examples of a secondary drier include a metal drier comprising an aluminum, a barium, a calcium, a cerium, an iron, a lanthanum, a lead, a manganese, a neodymium, a zinc, a zirconium, or a combination thereof. A rare earth drier comprises a lanthanum, a neodymium, a cerium, or a combination thereof.

In many embodiments, a coating may comprise from about 0.01% to about 0.1%, of an individual metal of a primary drier, by weight of the non-volatile component(s) of a coating composition. In many embodiments, a coating may comprise from about 0.1% to about 1.0%, of an individual metal of a secondary drier, by weight of the non-volatile component(s) of a coating composition. Standard physical and/or chemical properties for various driers comprising a metal (e.g., a calcium, a cerium, a cobalt, an iron, a lead, a manganese, a nickel, a rare earth, a zinc, a zirconium), and procedures for determining various metals' content for a driers are described in, for example, “ASTM Book of Standards, Volume 06.04, Paint—Solvents; Aromatic Hydrocarbons,” D600-90, 2002; and “Volume 06.01, Paint—Tests for Chemical, Physical, and Optical Properties; Appearance,” D2373-85, D2374-85, D2375-85, D2613-01, D3804-02, D3969-01, D3970-80, D3988-85, and D3989-01, 2002; and ASTM Book of Standards, Volume 06.01, Paint—Tests for Chemical, Physical, and Optical Properties; Appearance,” D564-87, 2002.

In embodiments wherein a secondary drier may be used, it may be combined with a primary drier, as the activity of a secondary drier are often limited when acting without the presence of a primary drier. Skinning refers to film-formation disproportionately at the coating-external environment interface. Skinning often results in wrinkle formation (“wrinkling”) in the film. A primary drier undesirably promotes skinning when acting without the presence of a secondary drier. In certain aspects, a zinc drier may be selected for reducing wrinkling in a thick film. In other aspects, a calcium drier and/or a zirconium drier may be selected instead of a lead drier, which may be limited due to an environmental law or regulation. In some facets, an iron drier, a rare earth drier, or a combination thereof, may be selected for use during film formation by baking. However, an iron drier may darken a coating. In further aspects, an aluminum drier may be selected for an alkyd-coating.

An alternative drier comprises a type of drier developed for use in a high solid and/or a water-borne coating, due to the inefficiency of a metal-soap drier in these types of coatings. Often, an alternative drier may be combined with a metal-soap drier. An example of a metal soap drier include a 1,10-phenanthronine, 2,2′-dipyridyl. A feeder drier comprises a type of drier designed to prolong the pot life of a coating in embodiments wherein a metal soap drier may be absorbed by a coating component such as a carbon black pigment, an organic red pigment, or a combination thereof. A feeder drier dissolves over time into the coating, thereby providing a continual supply of drier. An example a feeder drier includes a tradename composition such as a Troykyd® Perma Dry (Troy Corporation).

ii. Acids

An acid catalyzes amino resin cross-linking between a plurality of amino resins and/or an amino resin and an additional resin, though an acid may be more effective in promoting cross-linking between the additional resin and an amino resin. Examples of an acid include a strong acid, a weak acid, or a combination thereof. The rate of curing may be accelerated by selection of a strong acid over a weak acid. Examples of a strong acid include a p-toluenesulfonic acid (“PTSA”), a dodecylbenzenesulfonic acid (“DDBSA”), or a combination thereof. Examples of a weak acid include a phenyl acid phosphate (“PAP”), a butyl acid phosphate (“BAP”), or a combination thereof.

iii. Bases

A base catalyzes cross-linking between an acrylic resin and an epoxy resin in film formation. In specific aspects, the base comprises, for example, a dodecyl trimethyl ammonium chloride, a tri(dimethylaminomethyl)phenol, a melamine-formaldehyde resin, or a combination thereof.

iv. Urethane Catalysts

In specific aspects, a urethane coating comprises a catalyst to accelerate the reaction between an isocyanate moiety and a reactive hydrogen moiety. Examples of such a urethane catalyst include a tin compound, a zinc compound, a tertiary amine, or a combination thereof. Examples of a zinc compound include a zinc octoate, a zinc naphthenate, or a combination thereof. Examples of a tin compound include a dibutyltin dilaurate, a stannous octoate, or a combination thereof. An example of a tertiary amine includes a triethylene diamine.

g. Antiskinning Agent

An antiskinning agent comprises a composition, other than a drier, that reduces film-formation at the coating-external environment interface, reduce shrinkage (“wrinkling”), or a combination thereof. Such an antiskinning agent may be used to protect a coating from undesired film-formation after a container of coating has been opened, during normal film-formation, or a combination thereof. Examples of an antiskinning agent, with a commonly used coating concentration in parentheses, include a butyraloxime (about 0.2%), a cyclohexanone oxime, dipentene, an exkin 1, an exkin 2, an exkin 3, a guaiacol (about 0.001% to about 0.1%), a methyl ethyl ketoxime (about 0.2%), a pine oil (about 1% to about 2%), or a combination thereof. Generally, an antiskinning agent acts by reducing the rate of film-formation and/or promotes even film-formation throughout a coating by slowing an oxidative reaction that occurs as part of film formation. Examples of antioxidant antiskinning agent include a phenolic antioxidant, an oxime, or a combination thereof. Example of a phenolic antioxidant includes a guaiacol, a 4-tert-butylphenol, or a combination thereof. An oxime tends to evaporate such as during film formation, may be colorless, does not affect a coating's color property, and/or generally does not significantly alter the time of film-formation. Examples of an oxime include a butyraldoxime, a methyl ethyl ketoxime, a cyclohexanone oxime, or a combination thereof. In certain facets, an oxime may be used to slow skinning promoted by a copper drier.

h. Light Stabilizers

A coating, a film and/or a surface may be undesirably altered by contact with an environmental agent such as, for example, oxygen, pollution, water (e.g., moisture), and/or irradiation with light (e.g., UV light). To reduce such damaging alterations, a coating composition may comprise a light stabilizer. A light stabilizer (“stabilizer”) comprises a composition that reduces or prevents damage to a coating, film and/or surface by an environmental agent. Such agents may alter the color, cause a separation between two layers of film (“delamination”), promote chalking, promote crack formation, reduce gloss, or a combination thereof. This may be a particular problem for a film in an exterior environment, such as, for example, an automotive film. Additionally, a wood surface are susceptible to damage by an environmental agent (e.g., UV light).

Typically, a light stabilizer may comprise a UV absorber, a radical scavenger, or a combination thereof. A UV absorber comprises a composition that absorbs UV light. Examples of UV absorbers include a hydroxybenzophenone, a hydroxyphenylbenzotriazole, a hydrozyphenyl-5-triazine, an oxalic anilide, a yellow iron oxide, or a combination thereof. A hydroxyphenylbenzotriazole generally demonstrates the broadest range of UV wavelength absorption, and converts the absorbed UV light into heat. Additionally, a hydroxyphenylbenzotriazole and/or a hydrozyphenyl-5-triazine usually have the longest effective use in a film due to a higher resistance to photochemical reactions, relative to a hydroxybenzophenone and/or an oxalic anilide.

A radical scavenger light stabilizer (e.g., a sterically hindered amine) comprises a composition that chemically reacts with a chemical radical (“free radical”). Examples of a sterically hindered amine (“hindered amine light stabilizer,” “HALS”) include the ester derivatives of a decanedioic acid, such as a HALS I [“bis(1,2,2,6,6,-pentamethyl-4-poperidinyl) ester], which may be used in a non-acid catalyzed coating; and/or a HALS II [“bis(2,2,6,6,-tetramethyl-1-isooctyloxy-4-piperidinyl) ester], which may be used in an acid catalyzed coating.

For embodiments wherein a coating, film, and/or surface may be primarily located in-doors, a range of about 1% to about 3%, of a light stabilizer relative to binder content may be used. A range of about 1% to about 5%, of a light stabilizer relative to binder content may be used for exterior uses. Additionally, a combination of a UV absorber and a radical scavenger light stabilizer are contemplated in some embodiments, as the heat released by a UV absorber may promote radical formation. Light stabilizers are often commercially produced, and examples of UV absorber and/or a radical scavenger light stabilizer sold under a tradename include a Tinuvin® (Ciba Specialty Chemicals) and/or a Sanduvor® [Clariant LSM (America) Inc.].

i. Corrosion Inhibitors

A coating comprising a liquid component comprising water, particularly a water-borne coating, may promote corrosion in a container comprising iron, particularly at the lining, seams, handle, etc. A corrosion inhibitor reduces corrosion by water and/or an other chemical. Examples of a corrosion inhibitor includes a chromate, a phosphate, a molybdate, a wollastonite, a calcium ion-exchanged silica gel, a zinc compound, a borosilicate, a phosphosilicate, a hydrotalcite, or a combination thereof.

In certain embodiments, a corrosion inhibitor comprises an in-can corrosion inhibitor, a flash corrosion inhibitor, or a combination thereof. An in-can corrosion inhibitor (“can-corrosion inhibitor”) comprises a composition that reduces or prevents such corrosion. Examples of an in-can corrosion inhibitor are sodium nitrate, sodium benzoate, or a combination thereof. These compounds are typically used at a concentration of 1% each in a coating composition. In-can corrosion inhibitor are often commercially produced, and an example includes a SER-AD® FA179 (Condea Servo LLC.), typically used at about 0.3% in a coating composition. A flash corrosion inhibitor (“flash rust inhibitor”) comprises a composition that reduces or prevents corrosion produced by application of a coating comprising water to a metal surface (e.g., an iron surface). Often, an in-can corrosion inhibitor at an increased concentration may be added to a coating to act as a flash corrosion inhibitor. An example of a flash corrosion inhibitor includes a sodium nitrite, an ammonium benzoate, a 2-amino-2-methyl-propan-1-ol (“AMP”), a SER-AD® FA179 (Condea Servo LLC.), or a combination thereof. Standard procedures to determining the effectiveness of corrosion inhibition by a coating comprising a flash rust inhibitor are described, for example, in “ASTM Book of Standards, Volume 06.02, Paint—Products and Applications; Protective Coatings; Pipeline Coatings,” D5367-00, 2002.

j. Dehydrators

In some embodiments, preventing moisture from contacting a coating component such as a binder, a solvent, a pigment, or a combination thereof, may be desired. For example, certain urethane coatings undergo film-formation in the presence of moisture, as well as produce a film with increased yellowing, increased hazing and/or decreased gloss. A dehydrator may be added during coating production and/or storage to reduce contact with moisture. Examples of a dehydrator include an Additive TI (Bayer Corporation), an Additive OF (Bayer Corporation), or a combination thereof. An additive TI comprises a compound with one reactive isocyanate moiety, and it may be capable of reacting with a compound with a chemically reactive hydrogen such as water, an alcohol, a phenol, and/or an amide. However, in a reaction with water, the reaction products typically are carbon dioxide and a toluenesulfonamide. The toluenesulfonamide may be inert relative to a urethane binder, and/or soluble in many non-aqueous liquid components. In certain embodiments, a urethane coating may comprise about 0.5% to about 4% Additive TI. Additive OF comprises a dehydrator generally used in a urethane coating. In certain embodiments, a urethane coating may comprise about 1% to about 3% Additive OF.

k. Electrical Additives

In some embodiments, an additive alters an electrical property of a coating (e.g., electrical conductivity, electrical resistance). Examples of an additive to alter an electrical property of a coating and/or a coating component include an anti-static additive, an electrical resistance additive, or a combination thereof. An anti-static additive may be included in a coating comprising a flammable component to reduce the chance of an electrostatic spark occurring and igniting the coating. An anti-static additive comprises a composition that increases the electrical conductivity of a coating. An example of a flammable component comprises a hydrocarbon solvent. Examples of an anti-static additive include a Stadis® 425 (Octel-Starreon LLC USA), a Stadis® 450 (Octel-Starreon LLC USA), or a combination thereof. An electrical resistance additive comprises a composition that reduces the resistance to electricity by a coating. An electrical resistance additive may be included in a coating to improve the ability of a coating to be applied to a surface using an electrostatic spray applicator. For example, an oxygenated compound (e.g., a glycol ether) often possesses a high electrical conductivity, which may make use of an electrostatic spray applicator to apply a coating comprising an oxygenated compound relatively more difficult than a similar coating lacking an oxygenated compound. Examples of an electrical resistance additive include a Ramsprep, a Byk-ES 80 (BYK-Chemie GmbH), or a combination thereof. A Byk-ES 80 comprises, for example, an unsaturated acidic carboxylic acid ester alkylolammonium salt, and may be added between about 0.2% and about 2%, to a coating composition. Additionally, techniques in the art for determining an electrical property (e.g., electrical resistance) of a coating comprising an electrical additive may be used (see, for example, “ASTM Book of Standards, Volume 06.01, Paint—Tests for Chemical, Physical, and Optical Properties; Appearance,” D5682-95, 2002).

I. Anti-Insect Additives

Certain coatings may serve a protective role for a surface and/or a surrounding environment against insects, and thus may comprise an anti-insect agent. An example of a surface where a coating comprising an anti-insect agent may be used comprises a wooden surface. Examples of an area where coating comprising an anti-insect agent may be used may be a storage facility, such as a cargo hold of a ship and/or a railcar. An anti-insect agent comprises a composition that, upon contact, may be detrimental to the well-being (e.g., life, reproduction) of an invertebrate pest (e.g., an insect, an arachnid, etc), and may function as a biostatic and/or a biocide against such a pest. Examples of anti-insect additives that have been used in a coating include a copper naphthenate, a tributyl tin oxide, a zinc oxide, a 6-chloro epoxy hydroxy naphthalene, a 1-dichloro 2,2′bis-(p-chlorophenyl)ethane, or a combination thereof.

### P. COATING PREPARATION

A coating may comprise an insoluble particulate material. A particulate material may comprise a primary particle, an agglomerate, an aggregate, or a combination thereof. A primary particle comprises a single particle not in contact with a second particle. An agglomerate comprises two or more particles in contact with each other, and generally may be separated by a dispersion technique, a wetting agent, a dispersant, or a combination thereof. An aggregate comprises two or more particles in contact with each other, which are generally difficult to separate by a dispersion technique, a wetting agent, a dispersant, or a combination thereof.

Usually, a pigment, an extender, certain types of rheology modifiers, certain types of dispersants, or a combination thereof are the major sources of particulate material(s) in a coating. A cell-based particulate material generally may also be a source of particulate material in a coating. In certain embodiments, a cell-based particulate matter may be used in combination with and/or as a substitute for a pigment, an extender, a rheology modifier, a dispersant, or a combination thereof. In specific facets, a cell-based particulate matter may substitute for about 0.000001% to about 100%, of a pigment, an extender, a rheology modifier, a dispersant, or a combination thereof. In certain embodiments, a material formulation wherein the cell-based particulate material tends to be at or near the external environment interface of a material formulation. Preparation of such a material formulation wherein a particulate material may be at or near the external environment interface of a material formulation may be accomplished by formulation to enhance the ballooning, blooming, floating, flooding, etc. of the particulate material. Any technique used in the preparation of a coating comprising a pigment, an extender and/or any other form of particulate material described herein and/or in the art may be used in the preparation of a coating comprising the cell-based particulate material. Incorporation of particulate materials (e.g., pigments), assays for determining a rheological property and/or a related property (e.g., viscosity, flow, molecular weight, component concentration, particle size, particle shape, particle surface area, particle spread, dispersion, flocculation, solubility, oil absorption values, CPVC, hiding power, corrosion resistance, wet abrasion resistance, stain resistance, optical properties, porosity, surface tension, volatility, settling, leveling, sagging, slumping, draining, floating, flooding, cratering, foaming, splattering) of a coating component and/or a coating (e.g., pigment, binder, vehicle, surfactant, dispersant, paint) and procedures for determining such properties, as well as procedures for large scale (e.g., industrial) coating preparation (e.g., wetting, pigment dispersion into a vehicle, milling, letdown) are described in, for example, in Patton, T. C. “Paint Flow and Pigment Dispersion, A Rheological Approach to Coating and Ink Technology,” 1979.

In many embodiments, dispersion of the particulate material may be promoted by application of physical force (e.g., impact, shear) to the composition. Techniques such as grinding and/or milling are typically used to apply physical force for dispersion of particulate matter. Such an application of physical force may be used in the dispersal of the cell-based particulate material, such force may damage the structural integrity of the cell wall and/or cell membrane that confers size and/or shape to the material. The average particle size and/or shape may be altered by the degree of damage to the cell wall and/or cell membrane, which may alter a physical property, a chemical property, an optical property, or a combination thereof, of a cell-based particulate material. Examples of a physical property that may be altered by cell fragmentation include a rheological property, such as the contribution to viscosity, flow, etc., the tendency to form a primary particle, an agglomerate, an aggregate, etc. An example of a chemical property that may be altered includes allowing greater contact between a moieity such as an amine and/or a hydroxyl moiety(s) of internally located biomolecule(s) (e.g., a proteinaceous molecule) with a coating component, which may undergo a chemical reaction (e.g., cross-linking) with a binder. An example of an optical property that may be altered includes an alteration in the gloss characteristic of a coating and/or a film by a reduction in particle size due to cell fragmentation.

For example, during typical preparation of a water-borne and/or solvent-borne coating comprising particulate material such as a pigment and/or an extender, the particulate material may be dispersed into a paste known as a “grind” or “millbase.” A combination of a binder and a liquid component know as a “vehicle” may be used to disperse the particulate material into the grind. Often, a wetting additive may be included to promote dispersion of the particulate material. Additional vehicle and/or additive(s) are admixed with the grind in a stage referred to as the “letdown” to produce a coating of a desired composition and/or properties. These techniques and others for coating preparation in the art include, for example, in “ASTM Book of Standards, Volume 06.01, Paint—Tests for Chemical, Physical, and Optical Properties; Appearance,” D6619-00, 2002; in “Paint and Surface Coatings, Theory and Practice, Second Edition,” (Lambourne, R. and Strivens, T. A., Eds.), pp. 286-329, 1999; and in “Paints, Coatings and Solvents, Second, Completely Revised Edition,” (Stoye, D. and Freitag, W., Eds.) pp. 178-193, 1998. These techniques may be used in preparing a coating comprising the cell-based particulate matter, wherein the particulate matter may be treated as a pigment, an extender, and/or other such particulate material dispersed into a coating.

In another example, the effectiveness of the conversion of an agglomerate and/or an aggregate into a primary particle in the grind (e.g., a pigment, a pigment-vehicle combination, a paste), and latter stages (e.g., a lacquer, a paint) are typically measured to insure quality, using techniques such as, for example, those described in “ASTM Book of Standards, Volume 06.01, Paint—Tests for Chemical, Physical, and Optical Properties; Appearance,” D1210-96, 2002; “ASTM Book of Standards, Volume 06.02, Paint—Products and Applications; Protective Coatings; Pipeline Coatings,” D2338-02, D1316-93, and D2067-97, 2002; and in “ASTM Book of Standards, Volume 06.03, Paint—Pigments, Drying Oils, Polymers, Resins, Naval Stores, Cellulosic Esters, and Ink Vehicles,” D185-84, 2002. These techniques for the preparation of a coatings comprising a pigment, an extender, and/or other particulate material may be used in the preparation of a coating comprising a cell-based particulate material.

In a further example, a cell-based particulate material may be adapted for use in standard coating formulation techniques to improve a coating composition for desired properties. The pigment volume concentration is the volume of pigment in the total volume solids of a dry film. The volume solids is the fractional volume of a binder and a pigment in the total volume of a coating. In calculating the PVC, the content of a cell-based particulate material may be included in this and/or related calculations as a pigment and/or an extender. A related calculation to the PVC comprises the critical pigment volume concentration (“CPVC”), which refers to the formulation of a pigment and a binder wherein the coating comprises the minimum amount of binder to fill the voids between the pigment particles. A pigment to a binder concentration that exceeds the CVPC threshold produces a coating with empty spaces wherein gas (e.g., air, evaporated liquid component), may be trapped. Various properties rapidly change above the CPVC. For example, corrosion resistance, abrasion (e.g., scrub resistance), stain resistance, opacity, moisture resistance, rigidity, gloss, or a combination thereof, are more rapidly reduced above the CPVC, while reflectance may be increased. However, in certain embodiments, coating may be formulated above the CPVC and still produce a film suitable for given use upon a surface. Standard procedures for determining CPVC in the art may be used [see, for example, in “ASTM Book of Standards, Volume 06.01, Paint—Tests for Chemical, Physical, and Optical Properties; Appearance,” D1483-95, D281-95, and D6336-98, 2002; and in “Paint and Coating Testing Manual, Fourteenth Edition of the Gardner-Sward Handbook,” (Koleske, J. V. Ed.), pp. 252-258, 1995].

The physical and/or optical properties of a coating are affected by the size of a particulate material comprised within the coating. For example, inclusion of a physically hard particulate material, such as a silica extender, may increase the abrasion resistance of a film. In another example, gloss may be reduced when a particulate material of a larger average particle size increases the roughness of the surface of a coating and/or a film. Standard procedures for determining particle properties (e.g., size, shape) in the art may be used (see, for example, “ASTM Book of Standards, Volume 06.03, Paint—Pigments, Drying Oils, Polymers, Resins, Naval Stores, Cellulosic Esters, and Ink Vehicles,” D1366-86 and D3360-96, 2002; and in “Paint and Coating Testing Manual, Fourteenth Edition of the Gardner-Sward Handbook,” (Koleske, J. V. Ed.), pp. 305-332, 1995).

A biomolecular composition, particularly one prepared as a particulate and/or a powder material, may be incorporated into a powder coating. Specific procedures for determining the properties (e.g., particle size, surface coverage, optical properties) of a powder coating and/or a film have been described, for example, in “ASTM Book of Standards, Volume 06.02, Paint—Products and Applications; Protective Coatings; Pipeline Coatings,” D3451-01, D2967-02a, D4242-02, D5382-02 and D5861-95, 2002.

In some embodiments, the dispersion of particulate material (“fineness of grind”) in a coating is, in Hagman units (“Hu”), about 0.0 Hu to about 8.0 Hu. The dispersion of particulate material content of a coating may be empirically determined, for example, as described in “ASTM Book of Standards, Volume 06.01, Paint—Tests for Chemical, Physical, and Optical Properties; Appearance,” D1210-96, 2002. The size of particulate matter in a coating may affect gloss, with smaller particle size generally more conducive for a higher gloss property of a coating and/or a film. A whole cell particulate material may possess similar size and shape as the organism from which it was derived. For example, E. coli may be about 2 μm in length and about 0.8 μm in diameter, maize cells vary more in size, but a size of about 65 μm in diameter may be found in some cell types, and a Saccaromyces cerivsia may be about 10 μm in diameter. Of course, processing and purifying techniques may reduce the particle size by fragmentation of the cell wall and membrane, and a biomolecular composition may be prepared to an average particle size for a specific purpose (e.g., gloss). In certain facets, a visibly coarse and/or low gloss coating (e.g., a low gloss finish, a flat latex paint) has a dispersion of a particulate material of about 2.0 Hu to about 4.0 Hu. A particle size of about 100 μm to about 50 μm may be associated with a dispersion of about 0.0 Hu to about 4.0 Hu. In some aspects, a semi-gloss and/or a gloss coating has a dispersion of particulate material of about 5.0 Hu to about 7.5 Hu. A particle size of about 50 μm to about 40 μm; about 40 μm to about 26 μm; about 26 μm to about 13 μm; or about 13 μm to about 6 μm, may be associated with a dispersion of about 4.0 Hu to about 5.0 Hu; about 5.0 Hu to about 6.0 Hu; about 6.0 Hu to about 7.0 Hu; or about 7.0 Hu to about 7.5 Hu, respectively. In other aspects, a high gloss coating has a dispersion of particulate material of about 7.5 Hu to about 8.0 Hu. A particle size of about 6 μm to about 3 μm or about 3 μm to about 0.1 μm may be associated with a dispersion of about 7.5 Hu to about 7.75 Hu or about 7.75 Hu to about 8.0 Hu, respectively. In embodiments wherein a coating comprises a combination of particulate materials, wherein the different particulate materials such as a combination of a cell-based particulate material and one or more of different pigments, with each type of particulate material possessing a different average particle size, the gloss may be affected by the particle size of the largest type of particulate material added. However, gloss may also be empirically determined for a coating and/or a film, as described herein or by techniques in the art in light of the present disclosures.

### Q. EMPIRICALLY DETERMINING THE PROPERTIES OF COATINGS AND/OR FILMS

A coating and/or a film with a desired set of properties for a particular use may be prepared by varying the ranges and/or combinations of coating component(s), including a biomolecular composition described herein, and such coating selection and preparation may be done in light of the present disclosures. For example, a variety of assays are available to measure various properties of a coating, a coating application, and/or a film to determine the degree of suitability of a coating composition for use in a particular use (see, for example, in “Hess's Paint Film Defects: Their Causes and Cure,” 1979). In a further example, the physical properties (e.g., purity, density, solubility, volume solids and/or specific gravity, rheology, viscometry, and particle size) of the resulting a liquid paint and/or other coating product (e.g., on comprising a biomolecular composition), can be assessed using standard techniques of the art and/or as described in PAINT AND COATING TESTING MANUAL, 14^{th} ed. of the Gardner-Sward Handbook, J. V. Koleske, Editor (1995), American Society for Testing and Materials (ASTM), Ann Arbor, Mich., and applicable published ASTM assay methods. Alternatively, any other suitable assay method of the art, may be employed for assessing physical properties of the paint or coating mixture comprising an above-described biomolecular composition (e.g., an enzyme, an antifungal peptide additive, etc.). A paint and/or a coating comprising a biomolecular composition may then be assayed and used as described elsewhere herein, or the product may be employed for any other suitable purpose in the art in light of this disclosure.

General procedures for empirically determining the purity/properties of various coating components and/or coating compositions in the art may be used. Such procedures include measurement of density, volume solids and/or specific gravity, of a coating component and/or a coating composition, for purposes such as verification of component identity, aid in coating formulation, maintaining coating batch to batch consistency, etc. Examples of standard techniques for determining density of various solvents, liquids (e.g., a liquid coating), pigments, coatings (e.g., a powder coating) include those described in “ASTM Book of Standards, Volume 06.04, Paint—Solvents; Aromatic Hydrocarbons,” D2935-96, D1555M-00, D1555-95, and D3505-96, 2002; “ASTM Book of Standards, Volume 06.01, Paint—Tests for Chemical, Physical, and Optical Properties; Appearance,” D1475-98 and D215-91, 2002; “ASTM Book of Standards, Volume 06.03, Paint—Pigments, Drying Oils, Polymers, Resins, Naval Stores, Cellulosic Esters, and Ink Vehicles,” D153-84 and D153-84, 2002; “ASTM Book of Standards, Volume 06.02, Paint—Products and Applications; Protective Coatings; Pipeline Coatings,” D5965-02, 2002; and “Paint and Coating Testing Manual, Fourteenth Edition of the Gardner-Sward Handbook,” (Koleske, J. V. Ed.), pp. 289-304, 1995.

Standard surface specification and/or procedure(s) for preparing a surface (e.g., glass, wood, steel) for empirically measuring a physical and/or a visual property of a coating (e.g., a paint, a varnish, a lacquer) and/or a film are have been described (see, for example, “ASTM Book of Standards, Volume 06.01, Paint—Tests for Chemical, Physical, and Optical Properties; Appearance,” D3891-96, D609-00, and D2201-99, 2002; and “ASTM Book of Standards, Volume 06.02, Paint—Products and Applications; Protective Coatings; Pipeline Coatings,” D358-98, D4227-99, and D4228-99, 2002). Specific procedures for preparing a metal surface and an evaluating a coating (e.g., a primer, a paint) applied to a metal surface from the art may be used (see, for example, “ASTM Book of Standards, Volume 06.02, Paint—Products and Applications; Protective Coatings; Pipeline Coatings,” D3276-00, D5161-96, D4417-93, D3322-82, D2092-95, D5065-01, D5723-95, D6386-99, and D6492-99, 2002). Specific procedures for evaluating a coating applied to a plastic surface from the art may be used (see, for example, “ASTM Book of Standards, Volume 06.02, Paint—Products and Applications; Protective Coatings; Pipeline Coatings,” D3002-02, 2002).

Standard procedures for determining the stability of a coating (e.g., a water-borne coating, a UV irradiation cured coating) in a container prior and/or after opening the container from the art may be used (see, for example, “ASTM Book of Standards, Volume 06.02, Paint—Products and Applications; Protective Coatings; Pipeline Coatings,” D2243-95 and D4144-94, 2002).

Standard procedures for evaluating an applicator (e.g., a brush, a roller, a fabric, a spray applicator, an electrocoat bath) and/or a coating being applied by an applicator may be used (see, for example, “ASTM Book of Standards, Volume 06.02, Paint—Products and Applications; Protective Coatings; Pipeline Coatings,” D6737-01, D5913-96, D5959-96, D5301-92, D5068-02, D5069-92, D4707-97, D5286-01, D6337-98, D4285-83, and D5327-97, 2002; and “ASTM Book of Standards, Volume 06.01, Paint—Tests for Chemical, Physical, and Optical Properties; Appearance,” D1978-91, D5794-95, D4370-01, D4399-90, and D4584-86, 2002.

Standard procedures for preparing a coating (e.g., a paint, a varnish, a lacquer) and/or a film layer upon a surface for empirically measuring a physical and/or visual property may be used (see, for example, “ASTM Book of Standards, Volume 06.01, Paint—Tests for Chemical, Physical, and Optical Properties; Appearance,” D3924-80, D823-95, and D4708-99, 2002; “ASTM Book of Standards, Volume 06.02, Paint—Products and Applications; Protective Coatings; Pipeline Coatings,” D6206-97, D1734-93, and D4400-99, 2002; and “Paint and Coating Testing Manual, Fourteenth Edition of the Gardner-Sward Handbook,” (Koleske, J. V. Ed.), pp. 415-423, 1995.

Standard procedures for empirically determining the degree and duration of film formation of various coating compositions in the art may be used. Example of a standard technique for determining the degree/duration of film formation by loss of a volatile coating component and/or a cross-linking reaction for a coating (e.g., an oil-coating, a UV cured coating, a thermosetting powder coating) include those described in “ASTM Book of Standards, Volume 06.01, Paint—Tests for Chemical, Physical, and Optical Properties; Appearance,” D3539-87, D1640-95 and D5895-01e1, 2002; “ASTM Book of Standards, Volume 06.02, Paint—Products and Applications; Protective Coatings; Pipeline Coatings,” D4217-02, D3732-82, D2091-96, D711-89, D4752-98, and D5909-96a, 2002; “ASTM Book of Standards, Volume 06.03, Paint—Pigments, Drying Oils, Polymers, Resins, Naval Stores, Cellulosic Esters, and Ink Vehicles,” D2575-70 and D2354-98, 2002; and “Paint and Coating Testing Manual, Fourteenth Edition of the Gardner-Sward Handbook,” (Koleske, J. V. Ed.), pp. 407-414, 1995. Additionally, the temperature generated by a film formation reaction by a coating (e.g., a wood coating) may also be determined by techniques in the art (see, for example, “ASTM Book of Standards, Volume 06.02, Paint—Products and Applications; Protective Coatings; Pipeline Coatings,” D3259-95, 2002). Further, standard techniques for evaluating baking conditions on an organic coating and/or a film may be used (see, for example, “ASTM Book of Standards, Volume 06.01, Paint—Tests for Chemical, Physical, and Optical Properties; Appearance,” D2454-95, 2002).

In embodiments wherein film formation at ambient conditions may be used for a coating, a standard procedure in that art may be used for measuring film formation rate and/or stages (see for example, “ASTM Book of Standards, Volume 06.01, Paint—Tests for Chemical, Physical, and Optical Properties; Appearance,” D1640-95, 2002. In certain aspects wherein the ability of an oil to undergo film formation is to be determined, a standard procedure described in “ASTM Book of Standards, Volume 06.03, Paint—Pigments, Drying Oils, Polymers, Resins, Naval Stores, Cellulosic Esters, and Ink Vehicles,” D1955-85, 2002, may be used. In embodiments wherein the hardness of a film produced by a coating composition is measured (e.g., an organic coating), a standard procedure such as, for example, “ASTM Book of Standards, Volume 06.01, Paint—Tests for Chemical, Physical, and Optical Properties; Appearance,” D3363-00, D4366-95, and D1474-98, 2002.

Examples of a standard technique for determining the coating and/or the film thickness after application to various surface types are described in “ASTM Book of Standards, Volume 06.01, Paint—Tests for Chemical, Physical, and Optical Properties; Appearance,” D1212-91, D4414-95, D1005-95, D1400-00, D1186-01, and D6132-97, 2002; “ASTM Book of Standards, Volume 06.02, Paint—Products and Applications; Protective Coatings; Pipeline Coatings,” D5235-97, D4138-94, D2200-95, and D5796-99, 2002; and “Paint and Coating Testing Manual, Fourteenth Edition of the Gardner-Sward Handbook,” (Koleske, J. V. Ed.), pp. 424-438, 1995.

Examples of a standard technique for determining the adhesion of a coating and/or a film to various surface types are described in “ASTM Book of Standards, Volume 06.01, Paint—Tests for Chemical, Physical, and Optical Properties; Appearance,” D3359-02, D5179-98, and D2197-98, 2002; “ASTM Book of Standards, Volume 06.02, Paint—Products and Applications; Protective Coatings; Pipeline Coatings,” D4541-02 D3730-98, D4145-83, D4146-96, and D6677-01, 2002; and “Paint and Coating Testing Manual, Fourteenth Edition of the Gardner-Sward Handbook,” (Koleske, J. V. Ed.), pp. 513-524, 1995. Additionally, standard procedures for determining the ability of one or more layers of a multicoat system to function (e.g., adhere, weather) together are described in, for example, “ASTM Book of Standards, Volume 06.02, Paint—Products and Applications; Protective Coatings; Pipeline Coatings,” D5064-01, 2002.

Standard techniques for determining the physical properties (e.g., flexibility, tensile strength, toughness, impact resistance, hardness, mar resistance, blocking resistance) relevant to the durability of a film and/or the degree of film formation in the art may be used. Such procedures may be used to empirically characterize a film, and determine whether a coating composition produces a film suitable for a given application. Flexibility refers to the film's ability to undergo stress from bending and/or flexing without discernable damage (e.g., cracking). Examples of a standard technique for determining the flexibility of a film under mechanical or temperature stress are described in “ASTM Book of Standards, Volume 06.01, Paint—Tests for Chemical, Physical, and Optical Properties; Appearance,” D522-93a and D4145-83, 2002; “ASTM Book of Standards, Volume 06.02, Paint—Products and Applications; Protective Coatings; Pipeline Coatings,” D4145-83, D4146-96, and D1211-97, 2002; and “Paint and Coating Testing Manual, Fourteenth Edition of the Gardner-Sward Handbook,” (Koleske, J. V. Ed.), pp. 547-554, 1995. Related to flexibility is the tensile strength of a film, which refers to the ability of a film to undergo tensile deformation without developing discernable damage (e.g., cracking, tearing). Examples of a standard technique for determining the tensile strength of a film are described in “ASTM Book of Standards, Volume 06.01, Paint—Tests for Chemical, Physical, and Optical Properties; Appearance,” D2370-98 and D522-93a, 2002; and “Paint and Coating Testing Manual, Fourteenth Edition of the Gardner-Sward Handbook,” (Koleske, J. V. Ed.), pp. 534-545, 1995. Toughness refers to the film's ability to undergo strain imposed in a short period of time (e.g., one second or less) without discernable damage (e.g., breaking, tearing). Examples of a standard technique for determining the toughness of a film (e.g., a film for a pipeline) are described in “ASTM Book of Standards, Volume 06.01, Paint—Tests for Chemical, Physical, and Optical Properties; Appearance,” D2794-93, 2002; “ASTM Book of Standards, Volume 06.02, Paint—Products and Applications; Protective Coatings; Pipeline Coatings,” G14-88, 2002; and “Paint and Coating Testing Manual, Fourteenth Edition of the Gardner-Sward Handbook,” (Koleske, J. V. Ed.), pp. 547-554, 1995. Impact resistance refers to the ability of a film to undergo impact with an indenter without developing discernable damage at the dimple site (e.g., cracking). Examples of a standard technique for determining the impact resistance of a film (e.g., a film for a pipeline) are described in “ASTM Book of Standards, Volume 06.01, Paint—Tests for Chemical, Physical, and Optical Properties; Appearance,” D2794-93, 2002; “ASTM Book of Standards, Volume 06.02, Paint—Products and Applications; Protective Coatings; Pipeline Coatings,” G13-89 and G14-88, 2002; and “Paint and Coating Testing Manual, Fourteenth Edition of the Gardner-Sward Handbook,” (Koleske, J. V. Ed.), pp. 553-554, 1995. Hardness refers to the film's ability to undergo an applied static force without developing discernable damage (e.g., a scratch, an indentation). Examples of a standard technique for determining the hardness of a film are described in “ASTM Book of Standards, Volume 06.01, Paint—Tests for Chemical, Physical, and Optical Properties; Appearance” D1640-95, D1474-98, D2134-93, D4366-95, and D3363-00, 2002; and “Paint and Coating Testing Manual, Fourteenth Edition of the Gardner-Sward Handbook,” (Koleske, J. V. Ed.), pp. 555-584, 1995. Mar resistance (“mar abrasion resistance”) refers to the film's ability to undergo an applied dynamic force without developing a change in the film surface appearance (e.g., gloss) due to a permanent deformation (e.g., an indentation). Examples of a standard technique for determining the mar resistance of a film are described in “ASTM Book of Standards, Volume 06.01, Paint—Tests for Chemical, Physical, and Optical Properties; Appearance,” D5178-98 and D6037-96, 2002; and “Paint and Coating Testing Manual, Fourteenth Edition of the Gardner-Sward Handbook,” (Koleske, J. V. Ed.), pp. 525-533 and 579-584, 1995. Abrasion resistance (“wear abrasion resistance”) refers to the film's ability to undergo an applied dynamic force (e.g., washing) without removal of a film material. Examples of a standard technique for determining the abrasion resistance (e.g., burnish resistance) of a film are described in “ASTM Book of Standards, Volume 06.01, Paint—Tests for Chemical, Physical, and Optical Properties; Appearance,” D968-93 and D4060-01, 2002; “ASTM Book of Standards, Volume 06.02, Paint—Products and Applications; Protective Coatings; Pipeline Coatings,” D3170-01, D4213-96, D5181-91, D4828-94, D2486-00, D3450-00, D6736-01, and D6279-99e1, 2002; and “Paint and Coating Testing Manual, Fourteenth Edition of the Gardner-Sward Handbook,” (Koleske, J. V. Ed.), pp. 525-533, 1995. Blocking resistance (“block resistance”) refers to the ability of a film to resist adhering to a second film, particularly when the two films are pressed together (e.g., a coated door and coated doorframe). Examples of a standard technique for determining the blocking resistance of a film are described in “ASTM Book of Standards, Volume 06.02, Paint—Products and Applications; Protective Coatings; Pipeline Coatings,” D2793-99 and D3003-01, 2002. Abrasion resistance (“wear abrasion resistance”) refers to the film's ability to undergo an applied dynamic force (e.g., washing) without removal of film material. Slip resistance refers to a coating's (e.g., a floor coating) slipperiness, and may be evaluated as described in “Paint and Coating Testing Manual, Fourteenth Edition of the Gardner-Sward Handbook,” (Koleske, J. V. Ed.), pp. 600-606, 1995.

Weathering resistance refers to film's ability to endure and/or protect a surface from an external environmental condition. Examples of environmental conditions that may damage a film and/or a surface include contact with varying conditions of temperature, moisture, sunlight (e.g., UV resistance), pollution, biological organisms, or a combination thereof. Examples of a standard technique for determining the weathering resistance of a film (e.g., an automotive film, an external architectural film, a varnish, a wood coating, a steel coating) by evaluating the degree of damage (e.g., fungal growth, color alteration, dirt accumulation, gloss loss, chalking, cracking, blistering, flaking, erosion, surface rust), are described in “ASTM Book of Standards, Volume 06.01, Paint—Tests for Chemical, Physical, and Optical Properties; Appearance,” D4141-01, D1729-96, D660-93, D661-93, D662-93, D772-86, D4214-98, D3274-95, D714-02, D1654-92, D2244-02, D523-89, D1006-01, D1014-95, and D1186-01, 2002; “ASTM Book of Standards, Volume 06.02, Paint—Products and Applications; Protective Coatings; Pipeline Coatings,” D3719-00, D610-01, D1641-97, D2830-96, and D6763-02, 2002; and “Paint and Coating Testing Manual, Fourteenth Edition of the Gardner-Sward Handbook,” (Koleske, J. V. Ed.), pp. 619-642, 1995. Additionally, standard techniques in the art for determining the resistance of a film to artificial weathering conditions may be used. These procedures are used to contact a film with a simulated weathering condition (e.g., heat, moisture, light, UV irradiation) at an accelerated timetable are described in “ASTM Book of Standards, Volume 06.01, Paint—Tests for Chemical, Physical, and Optical Properties; Appearance,” D822-01, D4587-01, D5031-01, D6631-01, D6695-01, D5894-96, and D4141-01, 2002; “ASTM Book of Standards, Volume 06.02, Paint—Products and Applications; Protective Coatings; Pipeline Coatings,” D5722-95, D3361-01 and D3424-01, 2002; and “Paint and Coating Testing Manual, Fourteenth Edition of the Gardner-Sward Handbook” (Koleske, J. V. Ed.), pp. 643-653, 1995.

Standard techniques for determining a film's resistance to damage by various chemicals in the art may be used. Examples of a chemical that may be used in such procedures include an acid (e.g., about 3% acetic acid), a base, an alcohol (e.g., about 50% ethyl alcohol, hydrochloric acid, sulfuric acid), a detergent (e.g., a sodium phosphate solution), gasoline, a glycol based antifreeze, an oil (e.g., a vegetable oil, a lubricating petroleum oil, a grease), a solvent, water (e.g., a salt solution, a salt vapor), a polish abrasive, another coating (e.g., graffiti), or a combination thereof. Standard techniques for determining the chemical resistance of a film (e.g., an architectural film, an automotive film, a paint, a lacquer, a varnish, a traffic-coating, a metal surface-film) by evaluating possible damage (e.g., adhesion loss, alteration of gloss, blistering, discoloration, loss of hardness, staining, swelling, wrinkling) are described in, for example, “ASTM Book of Standards, Volume 06.02, Paint—Products and Applications; Protective Coatings; Pipeline Coatings,” D1308-02, D2571-95, D2792-69, D4752-98, D3260-01, D6137-97, D6686-01, D6688-01, and D6578-00, 2002; “ASTM Book of Standards, Volume 06.01, Paint—Tests for Chemical, Physical, and Optical Properties; Appearance,” D2370-98, D2248-01a, and D870-02, 2002; “ASTM Book of Standards, Volume 06.03, Paint—Pigments, Drying Oils, Polymers, Resins, Naval Stores, Cellulosic Esters, and Ink Vehicles,” D1647-89, 2002; and “Paint and Coating Testing Manual, Fourteenth Edition of the Gardner-Sward Handbook,” (Koleske, J. V. Ed.), pp. 662-666, 1995. Additionally, examples of a standard technique for determining the solvent resistance of a film are described in “ASTM Book of Standards, Volume 06.02, Paint—Products and Applications; Protective Coatings; Pipeline Coatings,” D4752-98 and D5402-93, 2002.

Standard techniques for determining a film's and/or a surface's (e.g., a metal, a wood) resistance to water permeability and/or damage (e.g., corrosion, blistering, adhesion reduction, hardness alteration, color alteration, gloss alteration) by contact with water and/or moisture are described in, for example, “ASTM Book of Standards, Volume 06.01, Paint—Tests for Chemical, Physical, and Optical Properties; Appearance,” D870-02, D1653-93, D1735-02, D2247-02, and D4585-99, 2002; and “ASTM Book of Standards, Volume 06.02, Paint—Products and Applications; Protective Coatings; Pipeline Coatings,” D2065-96, D2921-98, D3459-98, and D6665-01, 2002.

Standard techniques for determining a film's resistance to damage by a temperature greater than ambient condition in the art may be used. Thermal resistance refers to the film's ability to undergo stress from a temperature at or below 200° C. without discernable damage, while heat resistance refers to the film's ability to undergo stress from a temperature above 200° C. (e.g., fire resistance, fire retardancy, flame resistance) without discernable damage. Standard techniques for determining the thermal and/or heat resistance of a film (e.g., a metal-film, a wood-lacquer) by evaluating possible damage (e.g., adhesion loss, alteration of gloss, blistering, chalking, discoloration) are described in, for example, “ASTM Book of Standards, Volume 06.01, Paint—Tests for Chemical, Physical, and Optical Properties; Appearance,” D2370-98, D2485-91, D1360-98, D4206-96, and D3806-98, 2002; and “ASTM Book of Standards, Volume 06.02, Paint—Products and Applications; Protective Coatings; Pipeline Coatings,” D1211-97 and D6491-99, 2002.

In some embodiments, the component composition of a coating and/or a film may be measured to verify the presence, absence and/or amount of one or more coating components in a particular formulation. Standard procedures for sampling a coating and/or a film, and analyzing the material composition (e.g., a pigment, a binder, liquid component, toxic material), have been described in, for example, “ASTM Book of Standards, Volume 06.01, Paint—Tests for Chemical, Physical, and Optical Properties; Appearance,” D2371-85, D5380-93, D2372-85, D2698-90, D3723-84, D4451-02, D4563-02, D5145-90, D3925-02, D2348-02, D2245-90, D3624-85a, D3717-85a, D2349-90, D2350-90, D2351-90, D2352-85, D3271-87, D3272-76, D4017-02, D3792-99, D4457-02, D6133-00, D6191-97, D4764-01, D3718-85a, D3335-85a, D6580-00, E848-94, D4834-88, D4358-84, D2621-87, D3618-85a, D6438-99, D4359-90, D3168-85, and D4948-89, 2002; “ASTM Book of Standards, Volume 06.02, Paint—Products and Applications; Protective Coatings; Pipeline Coatings,” D5702-02, 2002; and “ASTM Book of Standards, Volume 06.03, Paint—Pigments, Drying Oils, Polymers, Resins, Naval Stores, Cellulosic Esters, and Ink Vehicles,” D1469-00, 2002.

The nonvolatile content of a coating component and/or a coating (“total solids content”) may provide an estimate, for example, of the volume of a film that may be produced by a coating component and/or a coating (e.g., a paint, a clear coating, an electrocoat bath applied coating, a binder solution, an emulsion, a varnish, an oil, a drier, a solvent) and/or the surface area a coating can cover relative to a film's thickness. The nonvolatile content of coating and/or a coating component may be determined by any technique known in the art (see, for example, “ASTM Book of Standards, Volume 06.01, Paint—Tests for Chemical, Physical, and Optical Properties; Appearance,” D6093-97, D2697-86, D1259-85, D1644-01, D2832-92, and D4209-82 D5145-90, 2002; “ASTM Book of Standards, Volume 06.02, Paint—Products and Applications; Protective Coatings; Pipeline Coatings,” D4713-92, D5095-91, 2002; and “ASTM Book of Standards, Volume 06.03, Paint—Pigments, Drying Oils, Polymers, Resins, Naval Stores, Cellulosic Esters, and Ink Vehicles,” D4139-82, 2002. Additionally, the volatile component of a coating may provide an estimate, for example, of VOC release and/or thermoplastic film formation time. The nonvolatile content of a coating component and/or a coating (e.g., a paint, a clear coating, an automotive coating, an emulsion, a binder solution, a varnish, an oil, a drier, a solvent) may be determined by any technique known in the art (see, for example, “ASTM Book of Standards, Volume 06.01, Paint—Tests for Chemical, Physical, and Optical Properties; Appearance,” D2369-01e1, D2832-92, D3960-02, D4140-82, D4209-82, D5087-02 and D6266-00a, 2002; and “ASTM Book of Standards, Volume 06.02, Paint—Products and Applications; Protective Coatings; Pipeline Coatings,” D5403-93, 2002.

Standard procedures for determining the visual appearance of a coating component, a coating and/or a film (e.g., reflectance, retroreflectance, fluorescence, photoluminescent light transmission, color, tinting strength, whiteness, measurement instruments, computerized data analysis) have been described, for example, in “ASTM Book of Standards, Volume 06.01, Paint—Tests for Chemical, Physical, and Optical Properties; Appearance,” E284-02b, E312-02, E805-01a, E179-96, E991-98, E1247-92, E308-01, E313-00, E808-01, E1336-96, E1341-96, E1347-97, E1360-90, D332-87, D387-00, E1455-97, E1477-98a, E1478-97 E1164-02, E1331-96, E1345-98, E1348-02, E1349-90, D5531-94, D3964-80, E1651-94, E1682-96, E1708-95, E1767-95, E1808-96, E1809-01, E2022-01, E2072-00, E2073-02, E2152-01, E2153-01, D1544-98, E259-98, D3022-84, D1535-01, E2175-01, E2214-02, and E2222-02, 2002; “ASTM Book of Standards, Volume 06.02, Paint—Products and Applications; Protective Coatings; Pipeline Coatings,” D4838-88 and D5326-94a, 2002; and “ASTM Book of Standards, Volume 06.03, Paint—Pigments, Drying Oils, Polymers, Resins, Naval Stores, Cellulosic Esters, and Ink Vehicles,” D2090-98, D2090-98 and D6166-97, 2002. Specific techniques for matching two or more colored coatings and/or coating components to reduce differences (e.g., metamerism) have been described, for example, in “ASTM Book of Standards, Volume 06.01, Paint—Tests for Chemical, Physical, and Optical Properties; Appearance,” D4086-92a, E1541-98 D2244-02 2002. Specific techniques for determining differences in the color of a coating and/or a coating component, particularly to insure color consistency of a coating composition, have been described, for example, in “ASTM Book of Standards, Volume 06.01, Paint—Tests for Chemical, Physical, and Optical Properties; Appearance,” D1729-96, D2616-96, E1499-97, and D3134-97, 2002.

Gloss refers to the film's “angular selectivity of reflectance, involving surface-reflected light, responsible for the degree to which reflected highlights or images of objects may be seen as superimposed on a surface” (“ASTM Book of Standards, Volume 06.01, Paint—Tests for Chemical, Physical, and Optical Properties; Appearance,” E284-02b, 2002). An example of a high gloss coating comprises a paint film with a glass-like surface appearance, as opposed to a low-gloss (“flat”) paint. Standard techniques for determining the gloss (e.g., specular gloss, sheen, haze, image clarity, waviness, directionality) of a coating and/or a film are described, for example, in “ASTM Book of Standards, Volume 06.01, Paint—Tests for Chemical, Physical, and Optical Properties; Appearance,” E284-02b, D523-89, D4449-90, E167-96, E430-97, D4039-93, D5767-95, and D2244-02, 2002; “ASTM Book of Standards, Volume 06.02, Paint—Products and Applications; Protective Coatings; Pipeline Coatings,” D3928-00a, 2002; and “Paint and Coating Testing Manual, Fourteenth Edition of the Gardner-Sward Handbook,” (Koleske, J. V. Ed.), pp. 470-480, 1995.

R. Removing a Coating or Film

In certain embodiments, a coating and/or a film may be removed from a surface include a non-film forming coating, a temporary film, a self-cleaning film, a coating and/or a film that has been damaged, may be otherwise no longer desired and/or no longer suitable for use. Various coating removers (e.g., a paint remover) in the art may be used, and often comprise solvents described herein capable of dissolving a coating component (e.g., a binder) integral to a film's structural integrity. Standard procedures for determining the effectiveness of a coating remover have been described, for example, in “ASTM Book of Standards, Volume 06.02, Paint—Products and Applications; Protective Coatings; Pipeline Coatings,” D6189-97, 2002.

### S. ELASTOMERS

An elastomer typically comprises a plurality of polymer chains with relatively weak attraction, and tend to form a more random structure. An elastomer may be processed by mastication, which comprises softening of a raw elastomer (e.g., a natural rubber) and/or pre-elastomer material often through mechanical action/shear, usually by using a mill machine and/or a chemical reaction with atmospheric oxygen, sometimes with the aid of a peptizer. An elastomer and/or pre-elastomer may undergo mixing with another component of the elastomeric material. An elastomer and/or pre-elastomeric material typically undergoes molding/shaping, and often may be processed using the techniques applicable for a plastic and/or a composite material (e.g., injection molding, centrifugal casting), though processing temperatures are often lower.

Vulcanization typically occurs after molding an elastomer into a shape (e.g., a part, an article) to maintain that shape. Vulcanization refers to creation of covalent cross-linking of an elastomer (e.g., a natural rubber, a synthetic rubber), and generally occurs at a double bond of an unsaturated polymer. An elastomer typically has some cross-links to prevent permanent deformation during use by increasing elasticity and/or decreasing plasticity. An elastomer typically has a cross-link about 4000 to about 10,000 monomer units in a polymer chain, though cross-links may occur up to at or nearly every monomer in a vulcanized elastomer chain. An elastomer often comprises a polymer chain of about 100,000 to about 1,000,000 molecular weight.

Often an elastomer comprises an additive such as a catalyst (e.g., a peroxide) to promote polymerization, a catalyst neutralizer, a chain transfer agent to control termination of one polymer chain and polymerization of another polymer chain, a filler (e.g., a carbon black, a barite, a clay, a chalk, a calcium carbonate, by a titanium dioxide), a reinforcement, an extender, a plasticizer (e.g., a chlorinated paraffin, an adipate, a linear dialkyl phthalate), a softener/processing aid (e.g., a wax such as a microcrystalline wax, a paraffin; an oil; a pitch, a synthetic organic ester), a vulcanized oil, an antioxidant (e.g., an antiozonant, particularly for an unsaturated elastomer), a blowing agent, a curing/vulcanization agent, a surfactant, an accelerator (e.g., a primary accelerator, a secondary accelerator), a fire retardant, a colorant, a retarder, a resin, a fatty acid (e.g., a stearic acid) and/or a fatty acid soap, a bonding agent, a wire (e.g., a brass coated steel wire), a fabric, or a combination thereof. An example of a curing/vulcanization agent includes a sulfur, a peroxide (e.g., an organic peroxide such as a dicumyl peroxide), a nitroso derivative, a maleimide, a phenolic resin, a quinone derivative, or a combination thereof.

A retarder inhibits premature vulcanization during preparation/processing, with examples including a benzoic acid; a N-(cyclohexylthio)phthalimide; a N-(trichloromethylthio)phthalimide; a N,N′,N″-hexaisopropylthimelamine; a N,N′,N″-tris(isopropylthio)-N,N′,N″-triphenylphosphoric triamide; a nitrosodiphenylamine; a phthalic anhydride; a salicylic acid; a sulfonamide derivative; or a combination thereof.

A peptizer promotes polymer (e.g., an isoprene-based rubber, a diene-based rubber) chain scission to reduce viscosity for ease of processing, enhance tack, improve dispersion of an additive, or a combination thereof. Examples of a peptizer include an aromatic bisulfate, a mercaptobenzothiazole, a mercaptan, or a combination thereof.

An accelerator may be used to accelerate vulcanization. Examples of an accelerator includes a delayed action accelerator (e.g., a mercaptobenzothiazole such as a 2-mercaptobenzothiazole); a dithiocarbamate (e.g., a zinc dithiocarbamate), a sulfur donor [e.g., a thiuram disulfide, a tetrabutylthiuram disulfide, a dipentamethylenethiuram tetrasulfide, a dipentamethylenethiuram disulfide, a tetraethylthiuram disulfide, a 2-(4-morpholinyldithio)benzothiazole]; a guanidine [e.g., a di(o-tolyl)-guanidine; a 1,3-diphenylguanidine], which may be used as a secondary accelerator in combination with mercaptobenzothiazole; a condensation reaction product of an aldehyde (e.g., an acetaldehyde, a formaldehyde, a butyraldehyde, a 2-ethylhexyl aldehyde) and an amine (e.g., a n-butylamine, an aniline, a p-toluidine), which may be used as a secondary accelerator in combination with another accelerator; or a combination thereof. An inert filler may be used to improve ease of handling and processing, particularly prior to vulcanization.

A hard rubber may be prepared from cross-linking an elastomer comprising a diene (e.g., a butadiene monomer), and often has a Young's modulus of about 315 to about 900 MPa, improved aging resistance, and chemical resistance (e.g., solvent resistance). An ebonite refers to a highly vulcanized hard rubber (e.g., about 500 MPa or greater Young's modulus, Shore D hardness of about 75). A hard rubber may be machined. A hard rubber typically may comprise an additive such as a preservative (e.g., ammonia), a vulcanization accelerator, a filler (e.g., a silica, a barayte, a chalk, a clay), a UV protector (e.g., a carbon black), a colorant (e.g., pigment), a softener (e.g., a wax, a pitch, an oil), or a combination thereof. A hard rubber may be processed into a rod, a tube, and/or a sheet; and often used in a chemical resistance application such as a chemical plant covering and/or lining; a battery box, a battery part; a paint brush bristle anchor; a chemical tank; a roller covering; a chemical resistant valve, a fitting, a pipe, and/or a pump; or a combination thereof.

An elastomer may comprise a chemically modified elastomer. A cyclized elastomer (e.g., a cyclized rubber) may be produced by contact with a strong acid and/or a Lewis acid (e.g., a titanium chloride, a ferric chloride, a sulfuric acid, a boron trifluoride, a stannic chloride, a p-toluenesulfonic acid). A cyclized elastomer may be used in an industrial roller, a hard molded product, a shoe sole, a reinforcement, a bonding agent, an ink, an adhesive, a coating, or a combination thereof. A hydrogenated (e.g., chlorinated, brominated, fluorinated) elastomer (e.g., a hydrogenated rubber) generally possesses enhanced crystallinity and improved ozone resistance. An elastomer (e.g., a rubber) may be surfaced halogenated by contact with a sodium hypochlorite and a weak acid, which may improve adhesion to a urethane paint; contact with a trichlrofluoromethane, which may improve heat resistance; contact with water comprising a bromine (e.g., a bromine salt) and a catalyst, which may improve the smoothness of the surface; contact with an antimony pentafluoride, which may reduce the surface friction coefficient; contact with a chlorine compound with irradiation, which generally decreases the friction coefficient and/or enhances aging resistance; or a combination thereof. A hydrohalogenated elastomer (e.g., a rubber hydrochloride) may be prepared by contact with a hydrogen chloride, and may be used in a polymeric film and/or a sheet application (e.g., a bonding layer between a metal/elastomer laminate; a laminate comprising a cellulose film, a metal foil, a paper). An elastomer may be alkylhalogenated by contact with an alkane comprising a bromine (e.g., CBrCl₃, CBr₄), and an alkylhalogenated elastomer (e.g., an alkylhalogenated rubber) generally possesses enhanced flame resistance, and often may be used in a hair pad, and/or in a liquid latex foam as a surface treatment/finish for a fiber (e.g., a carpet, a fabric). An elastomer (e.g., one comprising a double bond) may be epoxided by contact with a peracid (e.g., a performic acid), which generally produces a higher T_{g}. An epoxided elastomer (e.g., an epoxided rubber) may be used as a bonding agent between a PVC and an elastomer, and the epoxide may be used as a cross-linking and/or a graft polymerization reactive moiety. A meleated elastomer (e.g., a meleated rubber) may be produced by contact of an elastomer (e.g., one comprising a double bond) with a malic anhydride, typically in combination with a free radical initiator, to produce an anhydride moiety. A meleated elastomer may be capable of reacting (e.g., cross-linking) with an alcohol (e.g., diol), a diamine, a diisocyanate, a metal oxide, or a combination thereof, and the moiety may be used as a site for graft polymerization. An elastomer comprising a diene may be reacted with another compound comprising a diene. An elastomer may be the modified by a thiol and/or a sulfur by reaction with a double bond to cross-link, or a thiol may comprise a reactive moiety for an additional reaction. An elastomer may be reacted at the double bond with a nitrene and/or a carbene with a mixture of an aqueous sodium hydroxide/chloroform solution with a catalyst (e.g., a decyltrimethylammonium bromide), and a flame retardant chlorine moiety added by reaction with a halogenated nitrene and/or a halogenated carbene (e.g., a dichlorocarbene). An elastomer may be reacted with an aldehyde (e.g., a chloro aldehyde, a bromo aldehyde, a fluoro aldehyde, a formaldehyde, a glyoxal formaldehyde) with an acid catalyst. An elastomer may be graft copolymerized by contacting the elastomer with a monomer, and/or a polymer comprising a vinyl moiety (e.g., an acrylic such as a polymethyl methylacrylate, a polystyrene), usually in combination with a free radical based initiator and/or a catalyst. An elastomer-poly methyl methacrylate graft copolymer generally possesses impact resistance, and may be molded into article such as a roller-skate, a caster wheel, an electrical plug, and/or a cutting board; used in an adhesive/bonding agent between an elastomer, a textile, a metal, a leather, and/or a polyvinyl chloride; or a combination thereof. An elastomer may be depolymerized by chain scission often through oxidation, and may be used as a component in a composite (e.g., a bowling ball, a grinding wheel), an elastomer processing aid (e.g., a softener), a paint component, an adhesive/sealant, and/or an electrical insulation material.

An elastomer may be formed into an O-ring, a rope, and/or a sheet that may be cut, often for use in a gasket. A vulcanized and unvulcanized elastomer blend (“superior processing rubber”) generally possessed improve processing (e.g., extrusion) properties and dimensional stability, and may be used in the production of a polymeric film and/or a sheet, a shaped article, and/or an adhesive.

Specific assays may be used to determine the properties of an elastomer, though assays for properties of other polymeric material(s) may be used as applicable. All such assays may be used to aid in preparation, processing, post-cure, and/or manufacture of an elastomer; incorporation of a component (e.g., a biomolecule composition) of an elastomer such as by determining susceptibility of a polymeric material to a liquid component and/or heat for softening/melting prior to contact/admixing with a component (e.g., a biomolecule composition); evaluating the effect on an elastomers property by a component; or a combination thereof. Examples of assays more specific to an elastomer include those designed to measure and/or describe: compositional classes of elastomers and properties such as oil resistance (e.g., ASTM D 2000); component analysis of a rubber (e.g., ASTM D 297); rheological properties for an elastomer/rubber material for processing (e.g., ASTM D 6204); aging/weathering (i.e., about 10³ Pa to about 10⁸ Pa) heat resistance, oxygen resistance (e.g., ASTM D 572); weathering (i.e., atmosphere/ozone) resistance (e.g., ASTM D 1149, ASTM D 1171; ASTM D 750); UV/light resistance of a vulcanized rubber (e.g., ASTM D 1148 REV A); liquid resistance of an elastomer (e.g., ASTM D 471); gel characteristics, swelling index, and dilute solution viscosity of an elastomer/rubber contacted with a solvent (e.g., ASTM D 3616); fluid resistance of an elastomer/rubber gasket (e.g., ASTM F 146); gasket sealability (e.g., ASTM F 112); vulcanization and/or cure of a rubber (e.g., ASTM D 2084; ASTM D 5289); durability/crack resistance of a vulcanized rubber (e.g., ASTM D 813); mechanical properties of a vulcanized rubber (e.g., ASTM D 945); various properties (i.e., mechanical stability, Mooney viscosity, pH value, surface tension, carboxylic acid moiety(s) present on a polymer chain, total solids, viscosity, coagulum) (e.g., ASTM D 1417 REV A); fatigue in a vulcanized rubber (e.g., ASTM D 623); hardness of an elastomer (e.g., ASTM D 1415); shore D hardness of an elastomeric material and/or a plastic foam (e.g., ASTM D 2240); abrasion resistance (i.e., footwear) (e.g., ASTM D 1630); abrasion resistance of an elastomer/rubber (e.g., ASTM D 2228); tear strength of an elastomer (e.g., ASTM D 624); compression (e.g., gas compressive stress, liquid compressive stress) resistance for an elastomer (e.g., a seal, a machine mount, a vibration damper) (e.g., ASTM D 395); impact resistance (e.g., rebound) of a solid rubber (e.g., ASTM D 2632); viscoelastic properties of an elastomer at lower temperatures (e.g., ASTM D 1329); mooney viscosity/stress relaxation of an elastomer/rubber (e.g., ASTM D 1646); stress relaxation/force decay in compression of elastomers/rubbers (e.g., ASTM D 6147); stress relaxation moduli under various temperatures (i.e., about 23° C. to about 225° C.) (e.g., ASTM D 6048); vibration resistance/dynamic modulus over various temperatures (e.g., about −70° C. to about 200° C.) of an elastomer/rubber (e.g., ASTM D 5992); dynamic fatigue resistance (e.g., ASTM D 430); coefficient of linear thermal expansion of electrical insulating material (e.g., ASTM D 3386); heated air resistance of an elastomer (e.g., rubber) (e.g., ASTM D 573); oxidation while heated resistance (e.g., ASTM D 865); a rubbers adhesion property (e.g., ASTM D 429); electrical insulation properties of a pressure sensitive tape (e.g., ASTM D 1000); electrical insulation properties of a material (e.g., ASTM D 229, ASTM D 3638); dielectric strength loss by direct voltage stress (e.g., ASTM D 3755); electrical insulation of a wire and/or a cable jacket (e.g., ASTM D 2633); volume resistivity of an elastomer/rubber (e.g., ASTM D 991); staining (i.e., diffusion, contact, migration) of rubber contacting a surface (e.g., ASTM D 925); surface roughness of a material (e.g., ASTM F 1438); visual irregularity of an electrical protective rubber product (e.g., ASTM F 1236); adhesion of a rubber to a fabric, a metal, etc (e.g., ASTM D 413); or a combination thereof.

An example of an elastomer includes a thermoplastic elastomer, a melt processable rubber (“NPR”), a synthetic rubber (“SR”), a natural rubber (“NR”), a non-polymeric elastomer, or a combination thereof.

1. Thermoplastic Elastomers

A thermoplastic elastomer (“TPE”) refers to an elastomer typically comprising a thermoplastic monomer (e.g., a block copolymer comprising a thermoplastic segment and an elastomeric segment). A TPE typically may be processed by thermoplastic techniques such as extrusion, blow molding, injection molding, and/or thermoforming. A TPE typically possesses abrasion resistance, cutting resistance, scratch resistance, wear resistance, local strain resistance, and hardness. A TPE generally ranges from a softer durometer hardness grade (Shore A) to a harder grade (Shore D) (e.g., about Shore A 28 to about Shore D 82), overlapping the range of hardness for a thermoset rubber (e.g., about Shore A 22 to about a Shore A 96), and a thermoplastic (e.g., about a Shore A 48 to about Shore D 60). A TPE may comprise an additive (“property enhancer”) such as for example, a flame retardant, an electrical additive, a modifier, a stabilizer, or a combination thereof. A TPE membrane comprising a platinum catalyst may be used in a fuel cell membrane electrode. Examples of a TPE comprise an elastomeric polyolefin, a thermoplastic vulcanizate, a styrenic TPE, a thermoplastic polyurethane elastomer, a thermoplastic copolyester elastomer, a polyamide TPE, or a combination thereof.

a. Elastomeric Polyolefins

An elastomeric polyolefin generally comprises a copolymer (e.g., a block copolymer) comprising an olefin monomer, an elastomeric monomer, another olefin monomer that disrupts crystallinity, or a combination thereof. Examples of an elastomeric polyolefin comprise a thermoplastic polyolefin elastomer and/or a polyolefin elastomer. A thermoplastic polyolefin elastomer (“TPO elastomer”) typically comprises a polyolefin (e.g., a PP) thermoplastic segment, and an ethylene propylene diene “M” (“EPDM”) and/or an ethylene propylene rubber (“EPR”) as the elastomeric segment. A TPO elastomer may be processed by in mold assembly. A TPO elastomer may comprise an additive such as a UV absorber. A TPO elastomer may be blended with a thermoplastic polyolefin (e.g., a PE such as a LLDPE, a LDPE), a polyolefin elastomer, a polyolefin plastomer, an ethylene methylacrylate (“EMA”), an EVA, an ethylene ethylacrylate (“EEA”), a polybutene-1, an EPDM, or a combination thereof. A TPO elastomer blend with a thermoplastic polyolefin (e.g., a polyolefin copolymer), a polyolefin elastomer, a polyolefin plastomer, an EPDM, or a combination thereof, typically possesses improved UV resistance, aging resistance, toughness, low temperature properties (e.g., to about −40° C.), impact resistance, ozone resistance, and ductility. A TPO elastomer may be used in an automotive application such as a conveyor belt, a belt drive, a gasket, a grommet, a ducting, a bumper component, a mount for a motor, a side molding, a panel (e.g., a rocker panel), a window encapsulation, a dunnage, a seal (e.g., an O-ring, a lip seal), a plug, a brushing, a step pad, a fascia, a handle grip, a keypad, a roller, a caster, a noise/vibration/harshness application, a diaphragm, an interior skin, a boot, a connector, a sound deadening, and/or a bellow; a wire and/or cable application; a mechanical application; a biomedical application (e.g., an artificial heart pump material); a sporting good; or a combination thereof. A TPO elastomer may be used in a laminate (e.g., an automotive instrument panel) comprising, for example, an outer skin layer of TPO elastomer, a layer of a foamed polyolefin and/or a foamed PP, and a PP layer (“substrate layer”). A TPO elastomer comprising an ionomer copolymer may be used for an automotive application such as a skin for a dashboard and/or instrument panel.

Another example an elastomeric polyolefin comprises a polyolefin elastomer (“POE”), which comprises an olefin monomer (e.g., an ethylene) and another alpha-olefin monomer (e.g., an octene, a hexane, a butene) whose copolymerization reduces crystallinity. An example of a POE comprises an ethylene octene copolymer that may be flexible at about −40° C., possess UV stability, and may be cross-linked, and may be used in a cushioning component, a slipper bottom, a sandal, a work boot, a liner, a mat, an elastomeric foam, a rubber strip, a winter boot, a sock liner, a midsole, an automotive application (e.g., an air duct for an automotive interior, an interior trim, a bumper), a rub strip, a hose, a covering for wire insulation, a covering for a cable insulation, a low smoke emission jacket, a semiconductor shield, a flame retardant, an appliance wire, an impact modifier for another polymer (e.g., a PP), a noise/migration/harshness application material, or a combination thereof.

b. Thermoplastic Vulcanizates

A thermoplastic vulcanizate (“TPV”) typically comprises a thermoplastic olefin (e.g., a PP) polymer blend with a vulcanized rubber (e.g., an EPDM, an EPM, a butyl rubber, a nitrile rubber). A TPV's service temperatures often range from about −60° C. to about 150° C., though elongation generally increases with temperature while tensile strength and hardness decrease. A TPV may be used in an automotive application such as a conveyor belt, a belt drive, a gasket, a grommet, a ducting, a bumper component, a mount for a motor, a dunnage, a seal (e.g., an O-ring, a lip seal), a plug, a brushing, a step pad, a fascia, a handle grip, a keypad, a roller, a caster, a noise/vibration/harshness application, a diaphragm, an interior skin, a boot, a connector, a sound deadening, and/or a bellow.

A PP/EPDM TPV blend may be used in an appliance application such as a mount for a motor, a seal, a wheel, a vibration dampener, a roller, a gasket, a handle, and/or a diaphragm; an automotive application (e.g., an under the hood application) such as a weather stripping (e.g., a window weatherstripping), a boot/cover (e.g., a constant velocity joint boot), a wire covering, a cable covering, an air duct, a windshield component, a bumper component, a body seal (e.g., a door seal), a gasket, a hose, and/or a tube; an electrical application such as a switch boot, a mount for a motor shaft, a cable jacket, and/or a terminal plug; a building and/or a construction application such as a valve for irrigation, a connector for a welding line, a weather stripping, an expansion joint, and/or a seal for a sewer pipe; a biomedical application (e.g., a wound dressing, a drainage bag, a packaging for a pharmaceutical, a bed cover); a component for a business machine; a plumbing component; a hardware component; a power tool component; or a combination thereof. A PP/EPDM blend may be bonded to a polyamide (e.g., a nylon 6) for use in an automotive application (e.g., a driveshaft boot, an air induction system component, a tubing layer in a hydraulic oil hose). A PP/nitrile rubber has greater fuel resistance, oil resistance (e.g., hot oil resistance), and/or hot air resistance relative to a PP/EPDM; and may be used in an automotive application such as a filler gasket for fuel, an engine part (e.g., a tank liner, a mount), a hydraulic line, a carburetor component; or a combination thereof. A PP/butyl rubber blend may be known for sound dampening, vibration absorption, and/or gas and moisture barrier properties; and may be used in an application such as a calendered textile coating, a soft bellow, a sports ball (e.g., a football, a basketball, a soccer ball), a packaging seal; or a combination thereof.

c. Styrenic TPEs

A styrenic TPE (“styrene block copolymer”) generally comprises a styrene copolymer comprising an elastomeric monomer (e.g., a butadiene, an ethylene, an isoprene) and a harder thermoplastic monomer (e.g., about 30% styrene to about 40% styrene). The polymer typically comprises a block copolymer, often produced by anionic polymerization, with a segment of a hard monomer typically comprising about 50 to about 80 hard monomer units, while a segment of a soft monomer typically comprises about 20 to about 100 soft monomer units. An example of a styrenic TPE include a styrene-ethylene-butylene (“SEB”), a styrene-ethylene-butylenes-styrene (“SEBS”), a styrene-ethylene-propylene (“SEP”), a styrene-butadiene-styrene (“SBS”), a styrene-isoprene-styrene (“SIS”), or a combination thereof. A styrenic TPE may comprise an additive such as a heat stabilizer, and may be resistant to water, an acid, an alkali, though the resistance to a hydrocarbon solvent may be reduced. A styrenic TPE may be used in a wire covering; a cable covering; a footwear; a shoe sole; a sheet; a polymeric film (e.g., a biomedical disposable glove, a pharmaceutical application, a food application, a household application); a grip (e.g., a bike handle); a product for personal care; an utensil; a clear medical product; an adhesive (e.g., a hot melt adhesive, a pressure sensitive adhesive, an adhesive for a web coating); a sealant (e.g., used to attenuate noise and/or vibrations in a gasket); a window seal; a topper pad; a hospital pad; an automotive application (e.g., an interior pad, an insulation, a trim, a seating); a solution applied coating; a flexible oil gel; and/or an additive to a material formulation (e.g., a viscosity index improver used in a thermosetting resin modifier, a lube oil viscosity index improver, a thermoplastic modifier such as an impact modifier, an asphalt modifier).

A SEB typically has UV resistance, oxidation resistance (e.g., ozone resistance, oxygen resistance), and a service temperature up to about 177° C. A SEB may be processed similar to a PP, and may be used in a hospital product that may be resterilized. A SEBS may be blown and/or extruded molded into a polymeric film (e.g., a biomedical disposable glove, a pharmaceutical application, a food application, a household application). A SEB and/or a SEBS may comprise an aliphatic primary hydroxyl group at one or both of the terminal ends of the polymer, and may be used in preparation of an ink, a surfactant, a foam, a fiber, a coating, a sealant, an adhesive, and/or a polymer modifier. A SBS may be used as an impact modifier for a PS; an adhesive (e.g., a hot melt adhesive, a pressure sensitive adhesive); a polyolefin (e.g., a LLDPE) particularly for a polymeric film and/or a sheet; a HIPS; a biomedical product, a food container; or a combination thereof. A SIS may be processed similar to a PS, typically has a service temperature up to about 66° C., and may be used in a footwear and/or an adhesive. A SBS and/or a SIS may be used in formulation of a pressure sensitive adhesive (e.g., a tape adhesive, a label adhesive); a hot melt adhesive; a mastic; a sealant; a construction adhesive; an asphalt modifier (e.g., a pavement construction/repair binder, a joint sealant, a cracked sealant, a roofing membrane, a waterproofing membrane); used as an additive (e.g., a property enhancer) to improve the impact strength and/or toughness of a thermoplastic and/or a thermosetting resin up to about ambient temperatures; or a combination thereof.

A styrenic TPE comprising a polydiene (e.g., a SIS, a SBS) acts as a thermoplastic in processing above the T_{g} of a PS (e.g., about 95° C. to about 100° C.), and acts as cross-linked elastomer at a lowest temperature, so processing (e.g., extrusion, injection molding) often are about 100° C. to about 190° C. A styrenic TPE comprising a polydiene often comprises a filler (e.g., a silicate, a clay, a silica, CaO₃); a plasticizer (e.g., paraffinic oil); an antioxidant (e.g., a phosphitic antioxidant, a phenolic antioxidant); a stabilizer (e.g., dilauryldithiopropionate); a UV stabilizer (e.g., benzotriazine, benzophenone); a flow enhancer (e.g., a low molecular weight PE, zinc stearate, a microcrystalline wax); a pigment; a blowing agent; a combination thereof. A styrenic TPE comprising a polydiene may be blended with a polymer (e.g., a HIPS, a crystalline PS, a poly-alpha-methyl styrene, an EVA, a LDPE, a HDPE, a PP). A styrenic TPE comprising a polydiene may be used as an impact modifier for a thermoplastic and/or an asphalt; an adhesive (e.g., a pressure sensitive adhesive, a hot melt adhesive); a tubing; an O-ring; a gasket; a mat; an extruded hose; a swimming equipment (e.g., a rubberized suit, a snorkel, an eye mask, a fin, a raft); a footwear; a shoe sole; or a combination thereof.

d. Styrene Butadiene Rubbers

A styrene-butadiene rubber (“SBR”) comprises a copolymer (e.g., a random copolymer, a block copolymer) of a styrene and a butadiene, typically prepared by emulsion polymerization and/or solution polymerization. A SBR often comprises a capping agent and/or other chemical (e.g., a monomer). A SBR may comprise an additive such as a vulcanization agent and/or a filler (e.g., a silica, an aluminum silicate, a clay, a calcium silicate, a carbon black). A SBR produced from emulsion typically may be used in an automotive application (e.g., a tire, a sidewall, a tire tread), an industrial application (e.g., a wire and/or a cable covering, a roller), a hard molded product, a shoe sole, a reinforcement, a bonding agent, an ink, an adhesive (e.g., a pressure sensitive adhesive), and/or a coating. A SBR may be used in a hard rubber, a medical application, a toy, and/or a houseware. A SBR sometimes may be blended with a PVC and/or a NBR. A methacrylate-butadiene-styrene (“MDS”) terpolymer typically possesses clarity, weatherability, and heat stability; and may be used as an impact modifier particularly in a polymeric film and/or a sheet application (e.g., a packaging application).

e. Polyurethane Elastomers Such As Thermoplastic or Cast

A thermoplastic polyurethane (“TPU”) elastomer typically comprises a block copolymer comprising a hard segment comprising a diisocyanate (e.g., a MDI, a TDI, a 1,5-diisocyanate) and a chain extender (e.g., 1,4-butanediol, an ethylene glycol, a diamine); and a soft segment comprising a long chain diol (e.g., a polyether polyol, a polyester, a polycaprolactone polyester, a polyadipate polyester, a polytetramethylene glycol ether). An example of a polyether polyol includes a diol and/or a triol of about 4000 to about 6000 molecular weight. An example of a polyester includes a polyester prepared from a glycol (e.g., an ethylene glycol) and an adipic acid of about 2000 molecular weight and/or a poly(epsilon-caprolactone), and the polyester typically comprises a hydroxyl moiety at a termini. A TPU elastomer may be prepared from the diisocyanate reacted with the long chain diol and the chain extender. Cross-linking may occur by a peroxide curing agent. An example the catalyst commonly used includes an organotin and/or a tertiary amine. A TPU elastomer may be processed (e.g., extruded, casting, transfer molded, calendered, compression molded, in-mold assembly, injection molded, reaction injection molded, etc) at temperatures up to about 224° C. using equipment for rubber processing. A TPU elastomer typically has abrasion resistance, toughness, low temperature properties, tear resistance, aromatic oil resistance, and hydrocarbon resistance. A TPU elastomer may be used in a tubing (e.g., a waterline tubing, a fuel tubing); a hose line; a polymeric film (e.g., a lamination film, a film used in a diaper); a sheet; a belting; a footwear and/or a footwear component (e.g., an outer sole, a skate boot, a football cleat, a top lift, a ski boot,); a gasket; a grommet; a dust cover; a seal (e.g., a grease seal); a mechanical application (e.g., a gear); a wire covering; a cable covering; a golf ball cover; a wheel (e.g., an elevator wheel, a rollerskate wheel, an industrial wheel, a caster wheel, a skate board wheel); a hose jacket; an automotive application (e.g., an exterior automotive application) such as a body panel, a bumper (e.g., a bumper beam), a fascia, a cladding, a door, and/or an encapsulation for a window; an adhesive; a magnetic tape coating; or a combination thereof.

A polyester TPU may be resistant to oil, fuel, and/or a hydrocarbon solvent, and has applications such as a tube (e.g., a fuel line hose) and/or a clear polymeric film. A polyester TPU often may be blended with a thermoplastic (e.g., an ABS, a PVC, a PA, a SAN, a PC), typically to enhance a mechanical property, though the material may also comprise a plasticizer. A polyether TPU typically has fungal resistance, hydrolytic stability, toughness, and low temperature flexibility; and has application in a biomedical material. A UV resistance aliphatic polyether and/or a UV resistant aliphatic polyester may be used as a liner, a tubing, a polymeric film, a pipe, or a combination thereof. A PC/TPU elastomer may also be used in a profile, a wire covering, a cable covering, a sheet, a polymeric film, a tubing, an automotive application (e.g., an exterior automotive application), and/or a hose. A TPU elastomer may be blended with a PP and/or a SBC for use in an automotive application such as an instrument panel.

A polytetramethylene ether glycol TPU typically has excellent dielectric properties, fungal resistance, and hydrolysis resistance; and may be used in a wire covering; a cable covering; a reusable biomedical material and/or a biomedical device; a footwear material (e.g., an outer sole); a sneaker; a belting; a tubing; a caster wheel; an elastomeric film; or a combination thereof. A polycaprolactone TPU elastomer may be used in a gasket, an automotive panel, a belting, a seal, and/or a machine part. A polyadipate TPU elastomer may be used in a belting, a sheet, a polymeric film gasket, and/or a seal.

As an alternative to thermoplastic processing, a polyurethane elastomer may be prepared as a liquid prepolymer capable of being cast processed. A cast polyurethane elastomer typically comprises a TDI and/or a MDI prepolymer and a polyester and/or a polyether. A cast polyurethane elastomer typically may be used in a wheel (e.g., an elevator wheel, a forklift wheel, a rollerskate wheel, a wheel chock, a skateboard wheel); a mechanical and/or an industrial application (e.g., a thread protector for a drilling pipe, a chute for grain, a chute for coal, a shaft coupler, a conveyor belt, a gear, a pipeline pig, a pump liner, a shock absorber, a bumper pad, a papermill roller, a copier role, a steal roller, a drive belt, a sprocket, an O-ring, a hydraulic seal, a dental hammer, a sound dampening pad); a sleeve for a helicopter blade; a boat fender; an encapsulation (e.g., a gate valve encapsulation, a cattle tag encapsulation, a concrete mixer blade encapsulation); or a combination thereof.

f. Thermoplastic Copolyester Elastomers

A thermoplastic copolyester elastomer (“COPE,” “thermoplastic etherester elastomer,” “TEEE”) comprises a block copolymer comprising an amorphous soft segment and a polyester crystalline hard segment. A TEEE may be produced by condensation reaction. The reaction typically includes a polyalkylene ether glycol usually prepared from a tetramethylene oxide, a propylene oxide, an ethylene oxide, or a combination thereof, and a low molecular weight diol (e.g., a tetramethylene glycol, an ethylene glycol, a hexane diol, a butene diol, a 1,4-cyclohexanedimethanol) as the soft segment [e.g., a poly(oxytetramethylene terphthalate)]; and an aromatic dicarboxylic acid and/or the acid's methyl ester (e.g., a terphthalate acid such as a tetramethane terephthalate) reacted with a low molecular weight aliphatic diol to produce a hard segment [e.g., a poly(tetramethylene terphthalate)]. A TEEE may be processed by typical thermoplastic techniques (e.g., extrusion, injection molding, melt processing), as well as rotational molding, laminating, casting, and/or blow molding. A TEEE typically may have a Tₘ of about 196° C. or greater, and may be melt processed at temperatures of about 220° C. to about 260° C. A TEEE typically possesses good creep resistance; compression fatigue resistance; expansion strain resistance; flexural fatigue strength; heat resistance; hydrolysis resistance; and chemical resistance (e.g., an aqueous salt, a hydrocarbon, a nonpolar solvent), though a polar solvent may attack the elastomer at an elevated temperature, and meta cresol may dissolve the elastomer. An acid or a base may hydrolyze the polymer. A TEEE may often comprise an additive such as a filler (e.g., glass; a conductive filler such as a fiber coated with nickel, a stainless steel fiber, a carbon fiber, a carbon black), an internal lubricant (e.g., a silicone, a polytetrafluoro ethylene), a thickener and/or a thixotropic, an antiaging additive, an antioxidant (e.g., a secondary amine, a hindered polyphenol), or a combination thereof. A TEEE may be used as a modifier (e.g., an impact modifier) in another material formulation; a seal (e.g., an appliance seal); a molded air dam; a component of a power tool; a hose; a wire coating; a wire jacketing; a cable jacketing; a piece of camping equipment; a hydraulic tubing; a ski boot; a low-pressure tire (e.g., a snowmobile tire, a golf cart tire, a lawnmower tire); an automotive application such as a panel (e.g., an exterior panel part, a rocker panel), a spoiler, a fender extension, a spark plug boot, a fascia covering, a fascia, a wire covering, an extruded hose, a cable covering, a boot (e.g., an ignition boot), a bellow, a radiator panel, an exterior trim, a connector; or a combination thereof.

g. Polyamides

A polyamide TPE may be produced from reacting a polyol (e.g., a polyoxypropylene, a polyoxyethylene) and a polyamide. A polyamide TPE usually comprises a polyether block amide (“PEBA”), a polyester-amide, a polyamide (e.g., poly lauryl lactam)-ethylene-propylene (e.g., ethylene-propylene rubber), a polyamide acrylate graft copolymer, a polyetherester block copolymer (“polyetheresteramide”), or a combination thereof. For example, a PEBA block copolymer comprises an elastomeric segment (e.g., a polyether, a polyetherester, a polyester) and a polyamide thermoplastic segment. A polyamide TPE may be processed by extrusion, thermoforming, rotational molding, injection molding, and/or blow molding, with an example Tₘ of about 240° C. for an aromatic polyester amide and about 120° C. to about 205° C. for a polyesterether block copolymer. A polyamide TPE typically possesses good heat aging, a service temperature range up to about 150° C., and solvent resistance. A polyester amide TPE may retain properties such as modulus, tensile strength, elongation, and service temperature up to about 175° C. A PEBA generally possess hydrocarbon solvent resistance, cold-weather properties, UV stability, elastic memory, and reduced hysteresis. A polyamide ethylene-propylene typically possesses weather resistance, oil resistance, and fatigue resistance.

A polyamide TPE may comprise an additive such as a heat stabilizer. A polyamide TPE may be used for a watch case; sporting ball (e.g., a soccerball, a basketball, a volleyball); a footwear sole; an automotive application (e.g., a bellow, a wire covering); a flexible keypad; a hose for air-conditioning; an outerwear that may be waterproof and/or breathable (e.g., a respiratory device mouthpiece, a scuba equipment, a polymeric film for outerwear); a frame (e.g., a goggle frame, a ski frame, a swimming breaker frame); a handle cover, particularly for metal, handheld equipment due to nonslip adhesion (e.g., a control knob, an electric razor cover, a camera handle cover, a remote-control cover); or a combination thereof. A polyamide acrylic graft copolymer generally has a service temperature range of about −40° C. to about 165° C.; may be used in an optical fiber connector, an optical fiber sheathing, an automotive under-the-hood tubing, an automotive under-the-hood hose, a fastener (e.g., a snap fit fastener), a basket, and/or a seal; and often may be blended with a polyamide (e.g., a nylon 12) and/or a nitrile rubber.

2. Melt Processable Rubbers

A melt processable rubber (“MPR”) generally comprises an amorphous polymer, such as a polyolefin that has been halogenated (e.g., chlorinated). Often a MPR may be blended with an ethylene interpolymer to promote hydrogen bonding. A MPR generally lacks a well defined Tₘ, and applied sheer and heating (e.g., up to about 182° C.) may be used to process the material. A MPR may be calendered, extruded, injection molding, and/or compression molded. A MPR typically possesses chemical resistance, weather resistance, non-slip adhesive property, and a vibration absorption property. A MPR often comprises an additive such as a flame retardant, a stabilizer, a plasticizer, or a combination thereof. A MPR may comprise a cross linked polymer, particularly in a blend. A MPR may be used in a flexible keypad (e.g., computer keypad, a telephone keypad); a tube; a hosing; a polymeric film (e.g., a facemask); an automotive window seal; an automotive gasket (e.g., a fuel filter basket); a cable covering; a wire covering; an industrial window seal; an industrial door seal; an industrial weather stripping; a power tool housing; a handheld tool handle (e.g., a power tool handle); or a combination thereof.

3. Synthetic Rubbers

A synthetic rubber (“SR”) refers to a chemically manufactured elastomer such as a nitrile butadiene rubber, a butadiene rubber, a butyl rubber, a chlorosulfonated polyethylene, an epichlorohydrin, an ethylene propylene copolymer, a fluoroelastomer, a polyacrylate rubber, a poly(ethylene acrylic), a polychloroprene, a polyisoprene, a polysulfide rubber, a styrene butadiene rubber, a silicone rubber, a propylene oxide elastomer, an ethylene-vinyl acetate elastomer, or a combination thereof.

a. Nitrile Butadiene Rubbers

A nitrile butadiene rubber [“NBR,” “acrylonitrile butadiene copolymer,” “poly(acrylonitrile-co-1,3-butadiene) copolymer,” “butadiene acrylonitrile copolymer”] comprise a copolymer of acrylonitrile (e.g., about 20% to about 50%) and butadiene. The acrylonitrile monomer confers swelling resistance to a solvent (e.g., an aromatic solvent), a grease, water, a fuel (e.g., a gasoline), and/or an oil; but reduces low temperature flexibility. A NBR may be injection molded. A NBR generally possesses abrasion resistance and heat resistance. The backbone double bond may be hydrogenated to produce a hydrogenated nitrile rubber often used for an automotive application (e.g., an under the hood automotive application). A vulcanized NBR may have a service use up to 120° C. in air. A NBR often comprises an additive such as an antioxidant, a filler, a reinforcement, or a combination thereof. A NBR may be used in a low temperature seal; a low temperature O-ring; a shoe sole; a gasket; a sponge; a cable jacketing; a precision dynamic abrasion seal; a sheath and/or a covering for a wire and/or a cable; a polymeric film and/or a sheet application (e.g., a packaging); a hose and/or a tube (e.g., a hose and/or a tube for: an air conditioner, a fuel, a solvent, an oil); a belting; a footwear; a window seal; a gasketing for an appliance; a sheath and/or a covering for a wire and/or a cable; a material that contacts food (e.g., a creamery equipment); a fiction material composite (e.g., a break lining); an industrial application (e.g., a hydraulic equipment part, an oil well equipment part); an automotive application such as a tube (e.g., a fluid resistance tube; a fluid resistance tube, particularly a hydrocarbon resistant tube); a grease seal; an oil seal; an engine gasket; a hose (e.g., an inner hose for fuel system vent, an inner hose for a fuel filter neck); an impregnation resin (e.g., a textile impregnation resin, a paper impregnation resin, a leather impregnation resin); an adhesive; or a combination thereof. A NBR (e.g., a vulcanized NBR) may be combined (e.g., blended) with a polar thermoplastic (e.g., a PVC/ABS), a thermoplastic elastomer (e.g., a PVC/nitrile), or a combination thereof, and typically enhances a property such as compression set, oil resistance, material appearance, product tactile sensation, ease of processing, and/or reduced plasticizer migration (e.g., plasticizer blooming). A NBR blend with a thermoplastic elastomer may be used in a footwear, an automotive application such as a spoiler extension, a window frame, an armrest, a flexible lay flat, a weather stripping; an underground application such as a sheath and/or a covering for a wire and/or a cable; a hose (e.g., a hose for water, food, air, oil); or a combination thereof.

b. Butadiene Rubbers

A butadiene rubber (“BR,” “polybutadiene,” “PB”) may be polymerized from a 1,3-butadiene, and typically comprises a cis-1,4-polybutadiene, a trans-1,4-polybutadiene, or a combination thereof. Catalyst selection may alter cis content, as an alkyl-lithium catalyst produces about 40% cis-isomer content, a titanium catalyst produces about 92% cis-isomer, and a nickel and/or cobalt catalyst tends to produce about 97% cis-isomer content. A cis-1,4-polybutadiene typically has a low hysteresis, dynamic properties, and abrasion resistance. A trans-1,4-polybutadiene typically has thermal plasticity, toughness, and hardness relative to a cis-1,4-polybutadiene. A peroxide catalyst may be used to produce a thermoset by initiating cross-links at the vinyl moiety. A butadiene monomer such as a 2,3-dimethyl-1,3-butadiene, a 2-ethyl-1,3-butadiene, a 2-phenylbutadiene, a 1-methyl-1,3-butadiene, a 2-methylpentadiene, a 3-methylpentadiene, a 4-methylepentadiene, 1,3-cyclohexadiene, or a combination thereof, may be also used as a homopolymer and/or a copolymer (e.g., a 1,3-butadiene copolymer). A butadiene monomer such as a 1-methyl-1,3-butadiene (“pentadiene”) may be chemically modified (e.g., chlorinated, hydrogenated, phenolated, expoxidated, maleated), and used in copolymerization with another monomers to functionalize a polymer. Another monomer commonly used with a butadiene monomer includes a styrene, an isoprene, an acrylic monomer, an acrylonitrile, or a combination thereof. For example, a butadiene-acrylonitrile-methacrylic acid terpolymer has been used as a textile (e.g., a leather) finish. A BR may be processed by being calendered, casting, and/or extruded. A BR often may comprise an additive such as a filler (e.g., a precipitated silica, a high dispersal silica, a carbon black), a processing aid, an antioxidant, a curing agent, or a combination thereof. A BR may be used in an elastomer blend. A BR may be used in a sheet; a shoe sole; a shoe heel; a tubing; a golf ball; a hard rubber; a conveyor belt covering; a hose cover; a carcass stock; a V-belt; an electrical application; a sheath and/or a covering for a wire and/or a cable; an automotive application (e.g., a tire tread); or a combination thereof.

c. Butyl Rubbers

A butyl rubber typically comprises an isobutylene (e.g., 2-methyl-propene; about 98%) and a diolefin (e.g., an isoprene such as a 2-methyl-1,3-butadiene; often about 2%) copolymer (“IIR”); a terpolymer such as an isobutylene, p-methylstyrene, p-bromomethylstyrene terpolymer (“BIMS”); a polyisobutylene homopolymer; a copolymer of isobutylene and a n-butene (“polybutene”); or a combination thereof; often prepared using cationic polymerization with a Lewis acid (e.g., ALCl₃, BF₃), a Bronsted acid (e.g., HCl), and/or an alkyl halide [e.g., (CH₃)₃CCl]. A solid elastomer may be produced at a molecular weight of about 500,000. A butyl rubber typically comprises an additive such as a stabilizer (e.g., an antioxidant, an antiozonant, a calcium stearate to reduce dehydrohalogenation); a cross-linking/vulcanizing agent (e.g., a mercaptan, a divinylbenzene, sulfur); a curing agent; a processing aid; a filler (e.g., a clay, a silica, an aluminum silicate, carbon black, a calcium silicate); a plasticizer; or a combination thereof. A butyl rubber typically possesses resistance to environmental degradation (e.g., heat, humidity, bacteria), oxidation resistance, chemical resistance (e.g., a vegetable oil, an acetone, a glycol, water, an ethylene, a phosphate ester oil, a dilute mineral acid, a corrosive chemical), flexibility at low temperatures, and good electrical properties; but may be susceptible to a cyclohexane, a gasoline and/or a petroleum oil. A butyl rubber may be used in an automotive application such as a noise/vibration/harshness application (e.g., an engine mount, an automotive body mount), a sidewall (e.g., a white sidewall), a tube, an under the hood hose, a curing bladder, a cover strip, and/or a tire; a hard rubber; an electrical and/or an industrial application such as a wire and/or a cable covering; or a combination thereof. A low molecular weight, typically liquid, butyl rubber may be used in a caulking, a potting compound, a sealant, a coating, or a combination thereof. A depolymerized butyl rubber may be used in a sealant (e.g., an aquarium sealant), a liner for a reservoir, and/or a roofing coating. Various blends of a butyl rubber, a polybutylene, an EPDM, and/or a styrene butadiene rubber are typically used in a tire component.

An IIR often comprises a modified IIR, such as a halogenated (e.g., brominated, chlorinated, fluoridated) butyl rubber. An example of a halogenated butyl rubber includes a brominated butyl rubber (“BIIR,” “bromobutyl rubber”) and/or a chlorinated butyl rubber (“CIIR,” “chlorobutyl rubber”). A halogenated butyl rubber generally possesses skid resistance and/or rebound properties. A BIIR rubber typically has good chemical resistance to methanol, gasoline, and/or a brake fluid, and may be used in a break line. A BIIR generally possesses good flex resistance, and may be used in an automotive under-the-hood hose due to relatively better aging properties. A CIIR rubber typically has good barrier properties and flex resistance; and may be used in an automotive application such as a hose (e.g., an air-conditioning hose, a break line hose); a fuel line; a blend with EPDM rubber and NR to produce a white sidewall cover strip and/or a white sidewall tire; or a combination thereof.

d. Chlorinated/Chlorosulfonated Polyethylenes

An elastomer may be prepared from polyethylene upon a chlorination and/or a chlorosulfolyl substitution reaction using chlorine and sulfur dioxide. A chlorosulfonated polyethylene (“CSM”) typically comprises about 20% to about 40% chlorine and about 1% to about 2% sulfur (e.g., sulfonyl chloride). The sulfonyl chloride moiety may be used in a vulcanizing reaction and/or a curing reaction. A chlorinated polyethylene and/or a CSM often comprises an additive such as a vulcanization agent (e.g., a metal oxide), a filler (e.g., a clay, a silica, an aluminum silicate, carbon black, a calcium silicate), or a combination thereof. A CSM typically has oxygen resistance, ozone resistance, oil resistance, and heat resistance. A chlorinated polyethylene and/or a CSM may be used in a sheath and/or a covering for a wire and/or a cable; a hard rubber; an automotive application (e.g., an under the hood application) such as a fuel hose, a wire, a timing belt, a power steering hose, and/or a spark plug boot; or a combination thereof.

e. Epichlorohydrins

An epichlorohydrin typically comprises a polyether comprising a chloromethyloxirane (“ECH,” “1-chloro-2,3-epoxypropane”) polymer, a chloromethyloxirane oxirane copolymer (“ECO”), or a combination thereof. An epichlorohydrin may be produced by cationic polymerization using an alkylaluminum catalyst. An epichlorohydrin's chloromethyl moiety may participate in a curing reaction and/or a vulcanizing reaction. An epichlorohydrin typically has chemical resistance to an oil, an aliphatic solvent, and/or an aromatic fuel; acid resistance; alkaline resistance; flame resistance; fuel resistance; gas barrier properties; ozone resistance; and aging/weathering resistance. An epichlorohydrin may often comprise an additive (e.g., a flame retardant), a filler (e.g., a silica, an alumina, a reinforcing filler, a carbon black, a calcium carbonate, a clay, a talc), a plasticizer [e.g., a dioctyl phthalate, a di(butoxyethoxyethyl) formal], a vulcanizing agent, a process aid, a stabilizer (e.g., a heat stabilizer, an antioxidant), or a combination thereof. An epichlorohydrin may be used in a wire and/or a cable covering. An epichlorohydrin may be used as a copolymer (e.g., an electrostatic dissipation terpolymer) and/or a blend for an automotive application (e.g., an under the hood application) such as a hose, a gasket, a diaphragm for a fuel pump, a seal, and/or an engine mount.

f. Ethylene Propylene Copolymers

An ethylene propylene copolymer typically comprises a terpolymer comprising a propylene, an ethylene, and a non-conjugated diene (e.g., a dicyclopentadiene, a vinyl norbornene, an ethylidene norbornene) monomer (“EPDM”), a copolymer of an ethylene and a propylene [“EPM,” “ethylene propylene rubber,” “EP,” “EPR,” “poly(ethylene-co-propylene)”], or a combination thereof. An EPM and/or EPDM may be prepared using a metallocene and/or Zeigler Natta catalyst reaction. An ethylene-propylene copolymer may be branched. An EPDM [e.g., a poly(ethylene-co-propylene-co-5-ethylidene-2-norbornene] may be vulcanized due to the non-conjugated diene, and generally uses a curing agent (e.g., a dicyclopentadiene, a 1,4-hexadiene). An EPDM and/or an EPM generally have chemical resistance (e.g., a glycol, a nonpetroleum based brake fluid, a water, a salt, an oxygenated solvent), oxidation resistance, radiation resistance, service use at up to 105° C., and weather resistance. An EPM also typically has acid resistance, alkali resistance, detergent resistance, and age resistance. An EPDM generally has UV resistance, water alcohol mixture resistance, heat resistance, and ozone resistance. An EPDM and/or an EPM often may comprise an additive such as a filler (e.g., a calcium carbonate), a plasticizer, a reinforcement, or a combination thereof. An EPDM may comprise a coupling agent (e.g., a polyvinylamine, a polyacrylate acid) to promote bonding to a metal (e.g., a brass, an iron/steel, an aluminum). An EPDM may be graft copolymerized with a styrene and an acrylonitrile (“SAN-g-EPDM”). An EPDM backbone may also be chemically modified (e.g., maleated). A SAN-g-EPDM, a chemically modified EPDM, an EPDM, and/or an EPM may be used as an impact modifier. An EPDM and/or an EPM may be used in an automotive application such as a roofing, a tire, an exterior trim, a hose, a tube (e.g., a vacuum tube, a washer fluid tube), a weather stripping, a seal (e.g., a weather seal, a trunk lid seal, a body seal, a hood seal, a roof seal), a duct, a mount, a bumper, and/or a vibration dampening filler; a sealant (e.g., a construction sealant, an automotive sealant); an electrical application such as an encapsulating material for an electrical component, a sheath, a jacket and/or a covering for a wire and/or a cable; a waterproof membrane (e.g., a roofing membrane); or a combination thereof.

g. Fluoroelastomers

A fluoroelastomer (“FKM”) generally comprises a copolymer (e.g., a terpolymer) comprising a hexafluoroethylene, a hexafluoropropylene, a tetrafluoroethylene (“TFE”), a vinylidene fluoride, or a combination thereof. For example, a FKM terpolymer may comprises a vinylidene fluoride, a TFE, and a propylene; or a vinylidene fluoride, a TFE, and a hexafluoropropylene; or a vinylidene fluoride, a TFE, and a hexafluoroethylene. A FKM copolymer may comprise, for example, a TFE and a propylene; or a hexafluoropropylene and a vinylidene fluoride. A fluorophosphazene rubber comprises an elastomer prepared from a phosphazene comprising a perfluoralkoxy group attached to a backbone phosphorous, and may be considered herein as a fluoroelastomer. Examples of a fluoroelastomer include a poly(vinylidene fluoride-hexafluoropropylene); a poly(vinylidene fluoride-hexafluoropropylene-tetrachloroethylene); a poly[tetrachloroethylene-perfluoro(methyl vinyl ether)]; a poly[tetrachloroethylene-propylene]; a poly[vinylidene fluoride-chlorotrifluoroethylene]; a poly[vinylidene fluoride-tetrachloroethylene-perfluoro(methyl vinyl ether)]; such a polymer that may optionally comprise a comonomer for curing/cross-linking; or a combination thereof.

A FKM typically uses a curing agent (e.g., an anime, a bisphenol), and may be processed up to about 200° C. A FKM generally possesses chemical resistance (e.g., a hydrocarbon, a hydraulic fluid, a jet fuel, a lube oil, a gear lubricant, an engine oil, water, steam, an alcohol) that typically increases with increased fluorine content; good barrier properties to an oxygenated hydrocarbon, a gasoline, an alcohol, and/or an aromatic hydrocarbon; thermal resistance (e.g., up to about 250° C.); electrical resistance; and may possess resistance to an amine (e.g., an amine oil, an engine fluid additive). A FKM often may comprise an additive such as a thermal conductor (e.g., a zinc oxide), a heat resistor (e.g., a red iron oxide), a filler (e.g., a fine particle silica, a reinforcement), a curing agent (e.g., an accelerator), a processing aid, or a combination thereof. A FKM may be used in an aerospace application such as a cover gasket for a jet engine and/or an O-ring; an automotive application such as a gasket, an O-ring, a seal (e.g., an engine oil shaft seal), a drive train component, a chassis component (e.g., a gasket, a seal), a fuel delivery component such as a hose, a vapor line, an O-ring, a seal, and/or a fuel line, with a line and/or a hose often comprising an additional layer of a material such as a polyamide, an ethylene acrylic elastomer, a FEP (e.g., a Kevlar fiber reinforced FEP), or a combination thereof; a barrier to protect an electronic component; an oil equipment (e.g., an oil well equipment) application such as a seal, a jacket for a metal, and/or a down hole packer; a seal (e.g., an O-ring); a valve; a pump diaphragm; a gasket; a cable covering; a wire covering; a calendered stock; a polymeric film and/or a sheet; an additive (e.g., a viscosity improver) for another higher molecular weight polymer (e.g., a higher molecular weight FKM); a flange; a pipe; a valve lining; a chemical tank lining; a joint (e.g., a spool joint, a flue duct expansion joint, a flexible joint); and/or a combination thereof. A FKM may be blended with an additional polymer such as an elastomer (e.g., an EPR an EPDM, a nitrile, an epichlorohydrin, a silicone, a NBR, a fluorosilicone) and/or a thermoplastic (e.g., an ethylene acrylic copolymer), particularly to vulcanize with the additional polymer (e.g., a polymer that may be reacted with a FKM using a peroxide). A FKM/fluorosilicone blend may be used in an engine application such as an O-ring, a cylinder, a speedometer, a crankshaft, a valve, and/or a seal.

A copolymer of chlorotrifluoroethylene and polyvinylidene fluoride often comprises an elastomer, but may have properties of a flexible thermoplastic depending on the monomer content. A chlorotrifluoroethylene and polyvinylidene fluoride copolymer generally possesses tensile strength, tear strength, chemical resistance, low-temperature properties up to about −51° C., and thermal stability typically up to about 204° C. A chlorotrifluoroethylene and polyvinylidene fluoride copolymer may be processed by calendaring, dipping, and/or casting. A copolymer of chlorotrifluoroethylene and polyvinylidene fluoride generally may be used in a chemical resistant fabric, a hose, an O-ring, a glove, a gasket, and/or a pump impeller.

h. Polyacrylate Rubbers

A polyacrylate rubber (“ACM,” “acrylic rubber,” “acrylic elastomer”) polymer comprises an acrylic ester monomer such as a butyl acrylate (e.g., a n-butyl acrylate), an ethyl acrylate, a methoxyethyl acrylate (e.g., a 2-methoxyethyl acrylate), an ethoxyethyl acrylate, or a combination thereof; a monomer comprising a reactive moiety (e.g., a carboxyl, an epoxy, a chlorine) at about 1% to about 5% polymer content for cross-linking; and may also comprise an acrylonitrile monomer. An ACM may be processed by extrusion, compression molding, calendaring, injection molding, and/or resin transfer molding. An ACM often comprises an additive such as a reinforcement (e.g., a mineral, a carbon black), a plasticizer, a processing aid (e.g., a lubricant such as a stearic acid), an anti-heat aging additive (e.g., an anti-oxidant), a curing agent (e.g., a vulcanization agent), or a combination thereof. An ACM may be vulcanized using a metal carboxylate (e.g., a potassium stearate, a sodium stearate), a urea soap, a diamine, a trithiocyanuric acid, a sulfur moiety (e.g., a lead thiourea, an activated thiol, a sulfur soap), or a combination thereof. An ACM typically possesses heat resistance that allows flexibility and resistance to cracking from about −40° C. to about 204° C., ozone resistance, oil resistance, barrier properties against fuel vapors, compression set, and excellent oxygen resistance. An ACM may be used in an automotive application, such as a gasket.

i. Poly(Ethylene Acrylic(s)

A poly(ethylene acrylic) (“AEM”) comprises a terpolymer comprising a methyl acrylate monomer, an ethylene monomer, and a monomer comprising an acid moiety alkenoic acid) for cross-linking; and typically possesses chemical resistance, temperature resistance, and properties similar to an ACM. An AEM elastomer may be transfer molded, compression molded, and/or injection molded. An AEM elastomer often comprises a curing agent (e.g., a vulcanization agent such as a diamine, a peroxide diamine), a plasticizer, or a combination thereof. An AEM may be used an automotive application (e.g., an under the hood application) such as a gasket and/or a duct (e.g., an air intake duct); an industrial application such as a dampener (e.g., machinery dampener, a printer dampener), a seal (e.g., a hydraulic system seal, a pipe seal), a wire insulation for a motor lead; a wire jacketing and/or a cable jacketing; or a combination thereof.

j. Polychloroprenes

A polychloroprene (“CR,” “neoprene”) may be polymerized from a trans-2-chloro-2-butenylene, a cis-2-chloro-2-butenylene, a 2,3-dichlorobutadiene, or a combination thereof. A polychloroprene may be calendered and/or extruded. A CR typically possesses good chemical resistance (e.g., an oxidative chemical resistance, an oil resistance, grease resistance), wear resistance, high dynamic snap (i.e., flexing and twisting resistance); ignition resistance; noise/vibration/harshness dampening properties; flame retardance, self extinguishing property, and weather resistance, but may be susceptible to a fuel (e.g., a petroleum fuel). A polychloroprene often comprises an additive such as a filler (e.g., a clay, a silica, an aluminum silicate, carbon black, a calcium silicate), a processing aid, a vulcanization agent (e.g., a metal oxide), an accelerator, a retarder, a blowing agent, an antioxidant, or a combination thereof. A polychloroprene may be vulcanized using a Lewis acid. A polychloroprene may be used in an industrial application (e.g., a mining application); a gasket (e.g., a soil pipe gasket); a seal (e.g., a building seal, a concrete highway joint seal); a sheath, a jacket and/or a covering for a wire and/or a cable; a flame resistant application; an automotive application (e.g., an under the hood automotive application) such as a belt (e.g., a power transmission belt, an accessory belt, a valve timing belt), an air spring, a hose (e.g., a steering system hose, a coolant hose, a break hose), a seal (e.g., a vibration dampening mount seal), a shock absorber, a constant velocity joint boot, and/or a constant velocity joint liner; a hard rubber; a foamed elastomeric material; an adhesive; or a combination thereof.

k. Polyisoprenes

A polyisoprene (“IR,” “isoprene rubber”) may be produced by the polymerization of an isoprene (e.g., a 2-methyldivinyl, a 2-methyl-1,3-butadiene, a 2-methylerythrene). A trans-1,4-polyisoprene (“transpolyisoprene”) may be prepared using an alkylaluminum and a vanadium salt catalyst; while a cis-1,4-polyisoprene (“cispolyisoprene”) may be prepared using a trialkylaluminum and a titanium or an alkyllithium catalyst. An isoprene may be chemically modified (e.g., epoxidation, cyclization, oxidation, ozone lysis, hydrogenation, hydrohalogenation, halogenation, carbine addition) due to the double bond present in the monomer. A polyisoprene typically may be used in an automotive application such as an engine mount and/or a belting. A polyisoprene that has been depolymerized into a liquid may be used as a plasticizer. A thermoplastic may comprise a transpolyisoprene, and may be processed using injection molding, compression molding, calendaring, and/or extrusion. A transpolyisoprene often may comprise an additive such as a filler; and may be blended with an additional polymer. A transpolyisoprene may be used in an automotive application (e.g., a transmission belt); an industrial application (e.g., a cable covering); an adhesive (e.g., a hotmelt adhesive); a biomedical application (e.g., a splint, a cast, a prosthetic, a brace, an artificial limb attachment, an orthopedic device); a cover for a golf ball; or a combination thereof. A cispolyisoprene may be used in a tire, a mechanical application (e.g., a belt, a gasket); a polymeric film and/or a sheet application (e.g., a rubber sheeting); a sporting good; a footwear; a rubber band; a glove; a bottle nipple; a foamed rubber; a fiber; a sealant; a caulking; or a combination thereof.

l. Polysulfide Rubbers

A polysulfide rubber (“PSR”) monomer typically comprises a plurality of sulfur atoms separated by an organic compound, and a PSR may be produced by a condensation reaction of a polysulfide anion alkal metal salt (e.g., a sodium polysulfide such as a sodium tetrasulfide) and an organic dihalide [e.g., an organic dichloride such as a 1,2-dichloroethene, a bis(2-chloroethyl)ether, a propylene dichloride, a bis(2-chloroethyl) formal]. A branched PSR may be produced from dichloroethyl formal monomer in combination with a 1,2,3-trichloropropane; while a linear copolymer may a produced by using a methylene dichloride comonomer. A polysulfide typically comprises an additive such as a cross-linking/vulcanization agent (e.g., a 1,2,3-trichloropropane). A PSR may be extruded. A PSR typically has weather resistance, a service temperature range of about −55° C. to about 150° C., gas barrier property, water resistance, and solvent resistance (e.g., an ester, an alcohol, a ketone, some chlorinated solvents, an aliphatic liquid, a hydrocarbon solvent, a blend of an aliphatic and an aromatic solvent); but relatively low abrasion resistance and tensile strength. A PSR may be used in a hose for a chemical (e.g., a solvent), a metal coating, a concrete coating, a binder for a gasket, a printing roller, an electrical application (e.g., an electrical connector seal), a sealant (e.g., a fuel tank sealant, an electrical cable connection sealant), an adhesive, a component of a caulk (e.g., a deck caulking), a textile (e.g., leather) impregnation/finish to enhance solvent resistance and water resistance, or a combination thereof. A PS may be end capped with an epoxy resin and/or combined with an epoxy resin to act as a flexiblizer.

m. Silicone Rubbers

A silicone rubber (“SiR”) comprises a silicone atom in the polymer chain backbone, though an oxygen and/or a carbon may also be present in a monomer unit. A silicone rubber may be noted for a wide service temperature range (e.g., about −73° C. to about 300° C.), tear strength, compression set, and electrical properties. A silicone rubber may be used in an electrical application such as a cable covering; a semiconductor junction coating, an electrical insulator (e.g., a railway insulator); an encapsulation for an electrical component; an automotive application such as a gasket and/or a cable cover for an ignition cable; a surge arrestor; a biomedical application such as a shunt, catheter, a membrane, a surgical implant, an artificial heart, and/or a prosthesis (e.g., a tracheal prostheses, an ear prostheses, a bladder prosthesis, a pacemaker lead); or a combination thereof. A liquid silicone rubber (“LSR”) typically has a low compression set, an adhesion property, low hardness, and biocompatibility; and may be used a two pack material formulation (e.g., an adhesive, a sealant) that may be admixed (e.g., injection molded), a vent flap, and/or a door lock. A SiR may be blended with a polymer (e.g., a thermoplastic).

4. Natural Rubbers

A natural rubber (“NR”) may be chemically similar and/or the same as a synthetic rubber (i.e., a cispolyisoprene, a transpolyisoprene), though a NR may be isolated from a plant's sap (e.g., a tree such as a Hevea brasiliensis, a Taraxacum, a Parthenium argentatum) and generally comprises cis-polyisoprene as a dominant component. A NR may be processed by extrusion and/or molding. A NR typically possesses wear resistance, tear resistance, high tensile strength, resilience that may be greater than a synthetic rubber, low compression set, electrical properties, and chemical resistance to an acid or a base; but may soften above about 50° C., have a reduced resistance relative to a synthetic rubber to a lipid (e.g., a triglyceride oil, a petroleum fuel), and be soluble in a chlorinated solvent, an aliphatic solvent, and/or an aromatic solvent. A NR may be vulcanized, and may comprise a hard rubber (e.g., an ebonite). A natural rubber may comprise an additive such as a vulcanization agent (e.g., a sulfur), a vulcanization accelerator, a filler (e.g., a chalk, a silica, a barite, a clay, a carbon black, an aluminum silicate, a calcium silicate), a softener (e.g., a wax, an oil, a pitch), a stabilizer (e.g., an antioxidant, an antiozonant), a colorant (e.g., a pigment), a surface treatment (e.g., a wax), or a combination thereof. A natural rubber may be used in a mechanical application (e.g., a vibration reducing material), an electrical insulation material (e.g., a wire covering, a cable covering); an industrial application; a polymeric film and/or a sheet application; a tube; a bar; an automotive application such as an engine mount, a decoupler, a tire, and/or a tire tread; a tank lining; a printing roll; a latex thread; a rubber band; a baby bottle nipple; a shoe sole; a fiber; a glove; a tennis ball; an adhesive (e.g., a rubber cement); or a combination thereof. A depolymerized NR may be used in an artistic molding compound, a potting compound (e.g., an electrical application potting compound), a modifier for asphalt; or a combination thereof. A gutta-percha comprises a trans-polyisoprene isolated from a tropical tree sap (e.g., a Palaquim gutta, a Dichopsis gutta), and may be used in an adhesive, a golf ball, an orthodontic application (e.g., a dental filling), an additive for another elastomer, a transmission belting, and/or an electrical application (e.g., a wire covering). A transpolyisoprene may also be obtained from a Bolle tree.

An isoprene-based elastomer (e.g., a natural rubber, a polyisoprene) may be chemically modified by halogenation (e.g., fluorination, bromination, chlorination), typically by reaction of the halogen gas with a solvated (e.g., carbon tetrachloride solvated) elastomer. A chlorinated rubber (e.g., about 65% chlorine content) often has thermoplastic properties rather than elastomer properties, as well as flame resistance, chemical resistance, moisture resistance, mineral oil resistance, water resistance, and gasoline resistance. A chlorinated rubber often comprises an additive such as a plasticizer. A chlorinated rubber may be used to make a coating, an adhesive, a polymeric film and/or a sheet application, or a combination thereof.

5. Propylene Oxide Elastomers

A propylene oxide-allylglycidyl ether copolymer may have properties similar to a natural rubber, with susceptibility to a liquid component similar to a polychloroprene, and may be vulcanized with sulfur. A propylene oxide-allylglycidyl ether elastomer often comprises a filler (e.g., carbon black), a plasticizer, a stabilizer (e.g., a heat stabilizer, an antioxidant, an antiozonant), or a combination thereof. A propylene oxide-allylglycidyl ether elastomer may be used in an automotive application such as an engine mount and/or a suspension brushing.

6. Ethylene-Isoprene Elastomers

An ethylene-isoprene elastomer (“ethylene-isoprene rubber”) generally comprises an alternating copolymer prepared using a triisobutylaluminum catalyst.

7. Ethylene-Vinyl Acetate Elastomers

An ethylene-vinyl acetate copolymer comprising about 30% or greater vinyl acetate monomer generally becomes elastomeric, and may be used in a foam application, a wire and/or a cable covering, or a combination thereof.

8. Non-Polymeric Elastomers

Some elastomers are non-polymeric in nature and are contemplated for use with disclosures herein. Examples of a non-polymeric elastomer include a vulcanized oil.

a. Vulcanized Oils

A vulcanized oil comprises a triglyceride (e.g., a vegetable oil such as a soybean oil, a corn oil, a castor oil, a rapeseed oil) vulcanized, typically by reaction with sulfur, and may comprise an elastomer. An example of a vulcanized oil comprises a mineral rubber, which comprises a vulcanized oil and a bitumen (e.g., a gilsonite).

### T. ADHESIVES AND SEALANTS

An adhesive typically comprises a solid or a liquid, but converts into a solid final form (“set”) during normal use with desired attachment and material strength properties. For example, a liquid adhesive typically solidifies via a mechanism such as curing (i.e., a chemical reaction), cooling if molten, liquid component loss (e.g., evaporation, heating), or a combination thereof; while a solid adhesive may cure into a final solid form, or already be in a solid final form (e.g., a pressure sensitive adhesive).

An adhesive comprises an adhesive base (“base,” “binder”) from which the adhesive may be named, and the adhesive base confers the adherence and/or strength (i.e., stress load withstanding) properties to the adhesive. For example, an “epoxy adhesive” comprises an epoxy as the adhesive base. Often an adhesive base comprises a polymer and/or prepolymer (e.g., monomer, a shorter length polymer) that cures into a polymer (e.g., a polymer of the desired size range) and/or a cross-linked polymer.

In many embodiments, an adhesive may have a surface tension less than a surface tension of the surface of the adherent, which allows the adhesive to wet the surface for an attachment that may be sufficient to achieve the function of the adhesive. To “wet” or “wetting” in this context refers to creation of the intimate contact (e.g., a covalent bond, an ionic bond, a metallic bond, a van der Walls attraction) between two or more materials. Often the surface of the adherent comprises a polymeric material, a ceramic, a masonry, a glass, a wood, a metal, or a combination thereof. The surface tension (dyn/cm) of various possible attachable surfaces vary, as a metal may be relatively high (e.g., an aluminum may be about 500, a copper may be about 1000); while a cellulose may be about 45, and a polymer [e.g., an epoxy may be about 37, a polyamide may be about 46, a polycarbonate may be about 46, a polytetrafluoroethylene may be about 18, a silicone may be about 24) may be similar to a polymeric adhesive (e.g., a chlorinated epoxy resin adhesive may be about 33, an epoxy resin adhesive may be about 47). A polymeric adhesive often has a thermal expansion coefficient many fold greater than an adherent such as a metal, resulting in shrinkage that may cause failure of the bond(s) between the adhesive and an adherent, and a polymeric adhesive may to comprise a filler to reduce these thermal expansion differences.

A clean surface allows better wetting and attachment of the adhesive to the surface of the adherent. A surface may be prepared by chemically modification to promote adhesion, generally by reducing surface tension/enhancing wettability. Surface preparation techniques such as wiping a surface with a solvent, contacting a surface with a solvent vapor, cleaning a surface with an abrasive, cleaning a surface with a chemical (e.g., an acid), vapor-honing, ultrasonic cleaning, heating a surface (e.g., flame contact with the surface), plasma treatment of the surface, coronal discharge, contact with a metal, irradiation, grafting, etc., may be used prior to contact with an adhesive, a primer for an adhesive, or a combination thereof. For example, a polymeric material comprising a polyolefin (e.g., a polyethylene, a polypropylene) may be contacted and/or exposed to an electrical corona discharge; contacted with an acid (e.g., a chromate acid); contacted with a metal (e.g., a heated metal, an electrified metal); or a combination thereof; may introduce an oxygen comprising moiety (e.g., a carbonyl, a sulfonic acid, a carboxylic acid, a hydroxyl) as part of polymer. The moiety may promote adhesion to the polymeric material's surface. In another example a polymer comprising a fluorocarbon may be contacted with a chemical (e.g., an etchant) such as and a mixture of a sodium, tetrahydrofuran and naphthalene to introduce a polar moiety (e.g., a carboxyl, a carbonyl).

An adhesive may function as a sealant, a vibration dampener, an insulator, a gap filler, or a combination thereof. An adhesive may have a vibration dampening property, such as a noise dampening property, and/or an oscillation dampening property. An adhesive may function as a thermal insulator and/or an electrical insulator, though an adhesive comprising a conductive filler (e.g., a boron nitride filler, a silver filler) may be more electrically conductive and/or thermally conductive.

A polymeric adhesive typically also comprises a hardener (“curing agent”) that initiates a curing reaction. Examples of a hardener include an acid, an anhydride, and/or an amine. An adhesive may also comprise a catalyst to accelerate the chemical reaction between the base and the hardener. An adhesive sometimes comprises a liquid opponent (e.g., a solvent, often a combination of solvents) to formulate an adhesive in a spreadable consistency, reduce viscosity, or a combination thereof; though much (e.g., most) to about all of a solvent leaves (e.g., evaporates) the adhesive during conversion into a final solid form. An adhesive may comprise a diluent that lowers the base's concentration, typically for the purpose of aiding adhesive processing during formulation, lowering viscosity, or a combination thereof, and typically remains part of the adhesive by a reaction with the base during conversion into a solid form and/or being retained a polymeric material (e.g., a diluent that acts as a plasticizer). An adhesive may comprise a filler, typically a similar or the same as a filler described for a coating, a plastic, etc. to alter (e.g., improve, reduce) a property (e.g., permanence, shrinkage, thermal conduction, thermal resistance, strength, viscosity, electrical conduction, thermal expansion coefficient, etc.). An adhesive often comprises an antimicrobial agent. An adhesive (e.g., a pressure sensitive adhesive) may comprise a tackifier to enhance tackiness. A pressure sensitive adhesive generally comprises an amorphous network of high molecular weight molecule (e.g., a polymer) and a diluting resin (“tackifier”). Examples of the tackifier include an aliphatic petroleum resin, a rosen derivative resin, a terpene oligomer, an alkyl-modified phenolic resin, a coumarone-indene resin, or a combination thereof.

A “film adhesive” refers to a dry layer of an adhesive at the thickness of a polymeric film (“adhesive film”) and/or a sheet (“adhesive sheet”) generally capable of being cured by heat and/or pressure. A tape adhesive refers to an adhesive film and/or an adhesive sheet comprising a support material (e.g., a canvas, a cotton cloth, a vinyl backing material, a rubber backing material, a paper, a plastic film, a plastic sheet). The support material (e.g., a fabric) may be known as, in the context of an adhesive, a “reinforcement” or “carrier.” The support may be used to handle a semi-cured adhesive (e.g., a thermoset resin adhesive in B stage of cure) so the adhesive may be used as a tape adhesive, and/or temporarily separate the adhesive from an adherent. A film adhesive often comprises a pressure sensitive adhesive, which generally comprises a tacky adhesive at room temperature that flows when placed under finger and/or hand pressure to better contact and bind a surface, and may be manufactured comprising a pre-bound carrier (e.g., a paper, a plastic film, a metal foil), and often comprise a release coating (e.g., a silicone resin) to retard adhesion to the reverse side of the pre-bound carrier. Examples of the tape adhesive include a packaging tape, a masking sheet, and/or a postable paper note.

An adhesive may be classified by functional characteristics as either a structural adhesive or a nonstructural adhesive. A structural adhesive has a tensile and/or a sheer strength of about 1000 pounds per square inch (“psi”) or greater (e.g., about 5000 psi or greater), while a nonstructural adhesive functions for loads less than about 1000 psi (e.g., about 0.1 psi to about 1000 psi). A structural adhesive has permanence in function, such as being formulated for applications lasting up to 20 years and/or the expected service life of the joined adherents. A nonstructural adhesive may be used as a sealant, a hot melt adhesive, a wood glue, a pressure sensitive adhesive (e.g., a pressure sensitive tape), and/or a fastening in an assembly line production.

An adhesive may be classified by mold of curing and/or use. A pressure sensitive adhesive comprises a permanently tacky adhesive, and adheres to many surfaces upon application of a small pressure. A heat activated (“hot melt”) adhesive may be dry, but becomes tacky and/or fluid by heating, or heating in combination with pressure. A solvent activated adhesive comprises a dry adhesive that becomes tacky by contact with a liquid component (e.g., a solvent). A contact adhesive (“dry bond adhesive,” “contact bond adhesive”) generally remains dry to touch, but may be adhesive upon contact with the same or similar adhesive. An anaerobic adhesive cures in the absence of contact, or reduced contact, with air and/or oxygen. A solvent adhesive comprises a volatile liquid component, and becomes tacky and/or solidifies after solvent loss. A room temperature setting adhesive typically solidifies at about 20° C. to about 30° C.

An adhesive may be classified by composition as a thermoplastic adhesive, a thermoset adhesive (“thermosetting adhesive”), an elastomeric adhesive, or a combination thereof (e.g., “alloy blend adhesive,” “alloy adhesive,” “blend adhesive”). A thermoplastic adhesive and/or an elastomeric adhesive generally creeps under stress and/or suffers environmental degradation, and are more commonly used as a nonstructural adhesive. An elastomer adhesive (e.g., a pressure sensitive adhesive) typically possesses peel strength, impact resistance, fatigue resistance, and temperature resistance to about 94° C., but may creep at ambient conditions. An elastomer adhesive may be prepared in the form of a water-based latex cement and/or a solvent solution. In some embodiments, an elastomer adhesive comprises a mastic compound typically comprising a reclaimed rubber and/or a neoprene rubber; typically cure's by a loss of a solvent; and often may be used in a construction application such as to bind a wood frame to a flooring material (e.g., a gypsum board, a plywood board). An alloy adhesive and/or a thermoset adhesive often possess creep resistance, environmental resistance (e.g., heat resistance, oil resistance, solvent resistance, moisture resistance), physical properties (e.g., high strength), or a combination thereof, and are typically used as a structural adhesive(s).

Examples of adhesive include a thermoplastic adhesive, a thermoset adhesive, an elastomeric adhesive, an alloy adhesive, a non-polymeric adhesive, or a combination thereof. Examples of an adhesive includes a cellulosic adhesive, a cyanoacrylate adhesive, a dextrin adhesive, an ethylene-vinyl acetate copolymer adhesive, a melamine formaldehyde adhesive, a natural rubber adhesive, a neoprene/phenolic adhesive, a neoprene rubber adhesive, a nitrile rubber adhesive, a nitrile/phenolic adhesive, a phenolic adhesive, a phenol/resorcinol formaldehyde adhesive, a phenoxy adhesive, a polyamide adhesive, a polybenzimidazole adhesive, a polyethylene adhesive, a polyester adhesive, a polyimide adhesive, a polyisobutylene adhesive, a polysulfide adhesive, a polyurethane adhesive, a polyvinyl acetal adhesive, a polyvinyl acetal/phenolic adhesive, a polyvinyl acetate adhesive, a polyvinyl alcohol adhesive, a reclaimed rubber adhesive, a resorcinol adhesive, a silicone adhesive, a styrenic TPE adhesive, a styrene butadiene adhesive, a vinyl phenolic adhesive, a vinyl vinylidene adhesive, an acrylic acid diester adhesive, an epoxy adhesive, an epoxy/phenolic adhesive, an epoxy/polysulfide adhesive, a urea formaldehyde adhesive, a urea formaldehyde/melamine formaldehyde adhesive, a urea formaldehyde/phenol resorcinol adhesive, or a combination thereof. Examples of a thermosetting adhesive comprise an acrylic adhesive, an acrylic acid diester adhesive, a cyanoacrylate adhesive, a cyanate ester adhesive, an epoxy adhesive, a melamine formaldehyde adhesive, a phenolic adhesive, a polybenzimidazole adhesive, a polyester adhesive, a polyimide adhesive, a polyurethane adhesive, a resorcinol adhesive, a urea formaldehyde adhesive, or a combination thereof. Examples of a thermoplastic adhesive comprise an acrylic adhesive, an ethylene-vinyl acetate copolymer adhesive, a carbohydrate adhesive (e.g., a dextrin adhesive, a starch adhesive), a cellulosic adhesive (e.g., a cellulose acetate adhesive, cellulose acetate butyrate adhesive, cellulose nitrate adhesive), a polyethylene adhesive, a phenoxy adhesive, a polyamide adhesive, a polyvinyl acetal adhesive, a polyvinyl acetate adhesive, a polyvinyl alcohol adhesive, a protein adhesive (e.g., an animal adhesive, a soybean adhesive, a blood adhesive, a fish adhesive, a casein adhesive), a vinyl vinylidene adhesive, or a combination thereof. Examples of an elastomeric adhesive comprise a butyl rubber adhesive, a natural rubber adhesive, a neoprene rubber adhesive, a nitrile rubber adhesive, a polyisobutylene adhesive, a polysulfide adhesive, a reclaimed rubber adhesive, a silicone adhesive, a styrenic TPE adhesive, a styrene butadiene adhesive, or a combination thereof. Examples of an alloy adhesive comprise an epoxy/polyamide adhesive, an epoxy/phenolic adhesive, an epoxy/polysulfide adhesive, a neoprene/phenolic adhesive, a nitrile/phenolic adhesive, a phenol/resorcinol formaldehyde adhesive, a polyvinyl acetal/phenolic adhesive, a vinyl/phenolic adhesive, a urea formaldehyde/phenol resorcinol adhesive, a urea formaldehyde/melamine formaldehyde adhesive, or a combination thereof. Examples of a non-polymeric adhesive include a mucilage adhesive.

An adhesive may be classified by the method of application to a surface (e.g., a brushable adhesive, an extrudable adhesive, a spreadable adhesive, a trowelable adhesive, etc.); a flow property and/or a solidification property, such as a pressure sensitive adhesive which may flow by the application of pressure, an adhesive that hardens due to heat, an adhesive that hardens due to a chemical reaction, and/or an adhesive that hardens due to loss of a liquid component (e.g., solvent); the adhesive's adherent (e.g., a wood adhesive, a metal adhesive); a property of the adhesive (e.g., a weatherable adhesive, a heat-resistant adhesive, an acid-resistant adhesive); or a combination thereof.

An adhesive may comprise a sealant (e.g., a low performance sealant), by acting as a barrier to passage of a liquid, a gas (e.g., a fume, a flame, air, oxygen), an aerosol (e.g., smoke) a solid particle, an insect, or a combination thereof. A sealant may have a function such as act as a noise/vibration/harshness reducing material, maintain a gas and/or liquid pressure differential between a plurality of compartments, act as an electrical conductor, or a combination thereof. Often a sealant comprises an elastomeric material (e.g., an elastomeric polymer). A high-performance sealant may be capable of about 25% or greater (e.g., about 100%) compression and tension movements while adhering to the plurality of surfaces, and possesses about 80% or greater (e.g., about 100%) deformation recovery. A medium performance sealant may be capable of about 10% to about 25% compression and tension movements, while a low performing sealant may be capable of about 0.00001% to about 10% compression and tension movements, respectively. Often a high-performance sealant may be used as an exterior sealant, an interior sealant, a commercial building/construction application, a residential building/construction application, a gas pressure differential application (e.g., aerospace sealant), or a combination thereof. A medium performance sealant and/or a low performance sealant may be used in interior application, a commercial building/construction application, a residential building/construction application, or a combination thereof. A subtype of a sealant comprises a caulk, which may possess an aesthetic function, and may be used for that purpose, such as to improve the appearance of a joint. Many caulks are used for the traditional physical and/or mechanical functions of sealant.

Specific assay for an adhesive may be used to determine the properties of an adhesive and/or a sealant, though assays for properties of other polymeric material(s) may be used as applicable. All such assays may be used to aid in preparation, processing, post-cure, and/or manufacture of an adhesive; incorporation of a component of an adhesive (e.g., a biomolecule composition) such as by determining susceptibility to a liquid component; evaluate the effect on an adhesive's property by a component of an adhesive; or a combination thereof. Examples of assays more specific to an adhesive include, for example, those designed to measure and/or describe: an adhesive's storage life (e.g., ASTM D 1337); an adhesive's working life (e.g., ASTM D 1338); amylaceous (i.e., starch-like) matter content (e.g., ASTM D 1488); an adherent's preparation for an adhesive assay (e.g., ASTM D 2094); a surface's preparation for adhesive use (e.g., ASTM D 2651, ASTM D 3933, ASTM D 2674, ASTM D 2093); viscosity (e.g., ASTM D 2556, ASTM D 1084, ASTM D 3236); density (e.g., ASTM D 1875); a rubber cement's (e.g., reclaimed, natural, synthetic) properties (e.g., ASTM D 816); an adhesive's coverage/spreading on an adherent's surface (e.g., ASTM D 899, ASTM D 898); a nonvolatile component content of a urea-formaldehyde resin, a phenol, a resorcinol, a melamine, a dextrin, a starch, a casein, and/or an animal gelatin base adhesive (e.g., ASTM D 1490, ASTM D 1489, ASTM D 5040, ASTM D 1582); blocking point (e.g., ASTM D 1146); spot (i.e., simple/quick) adhesion (e.g., ASTM D 3808); tack (e.g., pressure sensitive adhesive tack) (e.g., ASTM D 3121, ASTM D 2979); cleavage strength and/or peel strength of an adhesive bond (e.g., ASTM D 1062, ASTM D 3807); shear fatigue by tension (e.g., ASTM D 3166); creep under shear, compressive loading, and/or temperature changes (e.g., ASTM D 2293, ASTM D 1780, ASTM D 2294); peel/stripping strength (e.g., ASTM D 1781, ASTM D 1876, ASTM D 903, ASTM D 3167); shear/shear strength properties at cryogenic temperatures (e.g., about −268° C. to about −55° C.; ASTM D 2557); sheer/tensile strength under tension loading at high temperatures (e.g., 315° C. to about 850° C.; ASTM D 2295); sheer and/or tensile strength under tension loading with an adherent (e.g., a laminate) (e.g., ASTM D 1002, ASTM D 3163, ASTM D 4027, ASTM D 3165, ASTM D 906, ASTM D 3528, ASTM D 1144, ASTM D 2339, ASTM D 905, ASTM D 3164, ASTM D 3983); shear strength of an adhesive bond that fill a gap (e.g., ASTM D 3931); flexural property such as flexural modulus, and/or flexural strength (e.g., ASTM D 3111); fracture strength in cleavage of an adhesive (e.g., ASTM D 3433); impact strength of an adhesive bond (e.g., ASTM D 950); compatibility with a plastic adherent by determination of stress cracking (e.g., ASTM D 3929); torque strength (e.g., ASTM D 3658); aging (i.e., oxygen resistance, irradiation/UV/visible light resistance, permanency) (e.g., ASTM D 1183, ASTM D 3632, ASTM D 1879, ASTM D 904); biodegradation (e.g., fungi) (e.g., ASTM D 4300); weathering/durability upon contact with moisture, water, air, temperature changes, physical stress (e.g., ASTM D 1151, ASTM D 2918, ASTM D 1828, ASTM D 2919; ASTM D 3762); chemical resistance of an adhesive bond (e.g., ASTM D 896); corrosivity of an adhesive (e.g., ASTM D 3310); an electrolytic corrosive property of an adhesive (e.g., ASTM D 3482); an electrical insulation property (e.g., ASTM D 1304); volume resistivity of a conductive adhesive (e.g., ASTM D 2739); the pH of an adhesive film (e.g., ASTM D 1583); an odor from an adhesive (e.g., ASTM D 4339); or a combination thereof.

1. Acrylic Adhesives

An acrylic adhesive typically comprises a thermoplastic and/or a thermosetting adhesive. An acrylic adhesive often comprises a monomer such as a 2-ethyhexyl acrylate, an acrylic acid, a vinyl acetate, an acrylamide, a dimethylaminoethyl methacrylic, a glycidyl methacrylic, an isoctyl acrylate, or a combination thereof. A thermoplastic acrylic adhesive may be prepared as a single emulsion, a multipack (e.g., a two pack) emulsion (e.g., a latex), and/or a solvent solution; and may comprise a catalyst. A thermoplastic acrylic adhesive typically has UV resistance, good bonding at low temperatures, but a relatively low heat resistance; and may be used to bind a textile, a metal (e.g., a metal foil) a plastic, a glass, a paper, or a combination thereof. A thermosetting acrylic adhesive typically comprises a multi-pack (e.g., a two-pack) liquid and/or paste adhesive comprising a hardener/catalyst that may be contacted with and/or admixed with the other component(s) to cure at an ambient and/or a baking condition. In some embodiments, the hardener/catalyst may be prepared as a liquid surface primer. A thermosetting acrylic adhesive typically possesses moisture resistance, weather resistance, and shear strength retention up to about 94° C., but a relatively low impact strength and peel strength; and may be used to bind a plastic, a wood, a metal, or a combination thereof. An acrylic adhesive may be used as a pressure sensitive adhesive and/or a sealant.

An acrylic sealant may comprise a silane (“siliconized acrylic adhesive”), and such an adhesive may function as a high performance adhesive, and may be used to bind an adherent such as a glass and/or an aluminum. An acrylic sealant often comprises a latex base, a plasticizer, a filler (e.g., a talc, a calcium carbonate, an aluminum silicate), a thixotropic, an anti-microbial agent (e.g., a mildewcide, a biocide), an antioxidant (e.g., a hindered phenol antioxidant), a UV absorber, an adhesion promoter (e.g., a surfactant, a silane), a liquid component (e.g., a minerals spirit, an ethylene glycol), or a combination thereof.

2. Acrylic Acid Diester Adhesives

An acrylic acid diester adhesive typically comprises a thermosetting adhesive prepared as a paste and/or a liquid. An acrylic acid diester adhesive may be an anaerobic adhesive, and generally cures at ambient conditions in the presence of a primer, but may require baking condition temperatures or hours of cure time without a primer. An acrylic acid diester adhesive generally possesses a service temperature range of about −54° C. to about 149° C.; and often may be used to bind an adherent such as a metal, a wood, a glass, a plastic, or a combination thereof.

3. Butyl Rubber Adhesives

A butyl rubber adhesive typically comprises an elastomeric adhesive prepared as a latex, a hot-melt, and/or a solvent based liquid that may cross-linkage via a curing agent, and typically sets at an ambient and/or a baking condition. A butyl rubber adhesive typically possesses water resistance, chemical resistance, good aging properties, a low permeability to a gas; but also tends to have low strength, and a low resistance to a hydrocarbon (e.g., an oil). A butyl rubber adhesive often may be used to bind a metal, an elastomer, a plastic (e.g., a plastic film, particularly a polyinylidene chloride, a polyethylene terephthalate), or a combination thereof. A butyl rubber sealant typically comprises an additive such as a filler (e.g., a carbon black, a silica, a clay, a calcium carbonate), a colorant (e.g., a zinc oxide, a titanium dioxide), a tackifier (e.g., a rosen-pentaerythritol ester), a thickener (e.g., a fiber), a liquid component/solvent (e.g., a cyclohexane), or a combination thereof.

4. Carbohydrate Adhesives

A carbohydrate adhesive comprises a carbohydrate-base (e.g., a starch, a dextrin). For example, a dextrin (“dextran”) adhesive comprises a thermoplastic adhesive prepared by reacting a starch (e.g., a short polymer starch) with HCl and a nitric acid at an elevated temperature up to about 125° C. A dextrin adhesive may comprise a filler (e.g., a clay). A dextrin adhesive typically used as a paper and/or a paperboard adhesive (e.g., postage stamp, an envelope, a gummed paper); as well as being used as an adhesive for a laminate.

5. Cellulosic Adhesives

A cellulosic adhesive (e.g., a cellulose acetate adhesive, a cellulose nitrate adhesive, a cellulose acetate butyrate adhesive) typically comprises a thermoplastic adhesive prepared as a solvent solution that may comprise a plasticizer. A cellulose nitrate adhesive tends to be flammable, more water resistant than another cellulosic adhesive, and may be used to bind an adherent such as a cloth, a plastic, a metal, a glass, or a combination thereof. A cellulose acetate adhesive and/or a cellulose acetate butyrate adhesive typically may be used to bind an adherent such as a paper, a fabric, a wood, a glass, a plastic, a leather, or a combination thereof.

6. Cyanoacrylate Adhesives

A cyanoacrylate (“cyanoacrylic ester”) (e.g., an allyl 2-cyanoacrylate, a methyl 2-cyanoacrylate, an ethyl 2-cyanoacrylate, a butyl 2-cyanoacrylate) adhesive comprises an anaerobic, thermosetting adhesive typically prepared as a liquid. A cyanoacrylate and a typically has reduced moisture resistance relative to an acrylic acid diester adhesive, a faster cure time (e.g., seconds), and a good bond strength with acidic surfaces being an exception, but typically has susceptibility to shock, heat, and/or a solvent. A cyanoacrylate adhesive typically binds a plastic, a metal, a glass, or a combination thereof.

7. Cyanate Ester Adhesives

A cyanate ester resin adhesive comprises of a thermosetting adhesive often used in a laminate (e.g., a microwave printed circuit board).

8. Epoxy Adhesives

A typical epoxy resin adhesive comprises a thermoset adhesive whose base comprises a bisphenol A and an epichlorohydrin that undergo reaction, and may be prepared as an one or multipart (e.g., a 2-pack) paste and/or liquid; or an one part paste or solid. A cure agent/hardener for ambient condition typically comprises a polyamide, an amine (e.g., a trimethylamine, a triethylamine, a triethylenetetraamine, a diethylenetriamine), or a combination thereof. An epoxy adhesive typically may cure at an ambient temperature to a baking condition (e.g., up to about 191° C.) temperature, with an epoxy adhesive that cures at a baking temperature generally possessing a greater material strength. A cure agent/hardener for an epoxy adhesive that cures at a baking condition temperature typically comprises an anhydride (e.g., a methyl nadic anhydride, a nadic anhydride) and/or a latent curing agent (e.g., a boron trifluoride monoethylamine). An epoxy adhesive may be used to bind an adherent such as a glass, a rubber, a wood, a plastic, a metal, a ceramic, or a combination thereof. An epoxy adhesive may comprise a filler. An epoxy adhesive typically possesses moisture resistance, oil resistance, solvent resistance, tensile-shear strength, creep resistance, and low cure shrinkage; but often possesses a low peel strength that may be improved by combination with another polymer (e.g., a polysulfide resin, a polyamide resin, a phenolic resin) in an alloy adhesive.

An epoxy-nylon (“epoxy-polyamide”) adhesive typically cures at a baking condition (e.g., about 177° C.); generally has good physical properties from a cryogenic temperature to about 83° C., peel strength, and sheer strength; and may be used in an aerospace application such as bonding an aluminum skin to an aircraft structure. An epoxy-phenolic adhesive generally cures at a baking condition (e.g., about 177° C.); generally possesses moisture resistance, oil resistance, solvent resistance, rigidity, sheer strength, and a continuous service temperature range up to about 177° C., but may have a reduced resistance to thermal shock and a low peel strength; and may be used to bind metal joints. An epoxy-polysulfide adhesive cures into a rubbery solid that typically possesses chemical resistance, flexibility, peel force resistance at low temperatures; and may be used as a general purpose sealant.

9. Melamine Formaldehyde Adhesives

A melamine formaldehyde adhesive typically comprises thermosetting adhesive prepared as a multi-pack (e.g., a two-part adhesive) and typically comprises a hardening agent, a filler/extender, or a combination thereof. A melamine formaldehyde adhesive typically solidifies under pressure at a baking condition temperature up to about 94° C.; and may be used to bind wood surfaces, such as the preparation of a plywood. A melamine formaldehyde adhesive may be blended with a urea formaldehyde base to reduce cost.

10. Natural Rubber Adhesives

A natural rubber adhesive typically comprises an elastomeric adhesive prepared as an one pack or a multi-pack (e.g., a two pack) latex and/or a solvent solution that may cure/cross-link at ambient conditions to a baking temperature. A natural rubber adhesive typically possesses strength, water resistance, moisture resistance, and tack, but generally has may be susceptible to an organic solvent. A natural rubber adhesive may be used as a rubber cement and/or a tape adhesive (e.g., a masking tape, a surgical tape, a duct tape). A natural rubber adhesive may be used to bind an adherent such as a wood, a metal, a fabric, a natural rubber, a masonite, a paper, a felt, or a combination thereof.

11. Neoprene Rubber Adhesives

A neoprene rubber adhesive (“neoprene adhesive”) typically comprises an elastomeric adhesive prepared as a solid, a solution, and/or a latex. A neoprene adhesive may comprise another polymer/resin, a filler, a metal oxide, or a combination thereof; and typically has strength, weather resistance, oil resistance, weak acid resistance, creep resistance, and a temperature resistance up to about 94° C. A neoprene adhesive may be used to bind an adherent such as a leather, a rubber (e.g., a neoprene), a plastic, a metal, a fabric, a wood, a fiber (e.g., a synthetic fiber), or a combination thereof.

12. Nitrile Rubber Adhesives

A nitrile rubber adhesive (“nitrile adhesive”) typically comprises an elastomeric adhesive prepared as a solvent solution and/or latex that solidifies via evaporation of the liquid component, pressure, heat, or a combination thereof. A nitrile adhesive typically comprises another polymer/resin (e.g., a thermosetting resin), a filler, a metal oxide, or a combination thereof; and typically has hydrocarbon solvent resistance and oil resistance, but a limited tack range. A nitrile adhesive typically may be used to bind an adherent such as a plastic (e.g., a vinyl plastic, a polyamide), a metal, a rubber (e.g., a nitrile rubber), a fiber, a wood, a combination thereof; but typically has weaker binding to a butyl rubber, a natural rubber, or a combination thereof.

13. Phenolic Adhesives

A phenolic adhesive (“phenoic resin adhesive”) (e.g., a phenolic formaldehyde adhesive) typically comprises a thermosetting adhesive that may be used to bind a wood adherent (e.g., a thermal insulation, an acoustic installation). A phenolic adhesive may be combined with a thermoplastic polymer (e.g., a polyvinyl polymer), a synthetic rubber (e.g., a nitrile rubber), or a combination thereof, to enhance flexibility, expand application use to an additional adherent, or a combination thereof.

A neoprene-phenolic adhesive comprises a phenolic resin and a neoprene resin typically prepared as a film adhesive and/or a solvent solution. A neoprene-phenolic adhesive may be solidified by curing at about 149° C. under pressure (e.g., several atmospheres of pressure); and generally possesses a service temperature of about −57° C. to about 94° C., impact strength, fatigue strength, and creep resistance, though the sheer strength may be lower than another phenolic adhesive. A neoprene-phenolic adhesive may be used as a general purpose adhesive, but may be used to bind a plastic, a glass, a metal, or a combination thereof.

A nitrile-phenolic adhesive comprises a phenolic resin and a nitrile rubber, and may be prepared as a film adhesive (e.g.; a carrier supported film adhesive) and/or a solvent solution, and may be solidified by baking temperatures up to about 149° C. to about 260° C. under pressure (e.g., over 10 atmospheres of pressure). A nitrile-phenolic adhesive typically has a service temperature up to about 149° C., sheer strength, peel strength, oil resistance, solvent resistance, water resistance, fatigue resistance, impact strength, and creep resistance; and may be used to bind a glass, a plastic, a rubber, a metal, or a combination thereof, with particular effectiveness typically on a metal surface.

A vinyl-phenolic adhesive comprises a blend of a phenolic resin and a polyvinyl resin (e.g., a polyvinyl butyral resin, a polyvinyl formal resin) and may be prepared as a liquid (e.g., a solvent solution, an emulsion), a tape, a powder, and/or a film adhesive (e.g., a carrier supported film adhesive); and typically cures at a baking condition temperature, often under pressure. A vinyl-phenolic adhesive generally possesses impact resistance, chemical resistance, solvent resistance, oil resistance, water resistance, weather resistance, peel strength, sheer strength, heat resistance, and a service temperature up to about 94° C.; and may be used to bond a plastic, a metal, an elastomer, or a combination thereof (e.g., a printed circuit board components comprising a plastic laminate bonded to a copper sheet).

14. Phenoxy Adhesives

A phenoxy adhesive typically comprises a thermoplastic adhesive prepared as a hot melt solid, a solvent solution, and/or a film, and typically cured by heat and/or pressure. A phenoxy adhesive generally retains strength and creep resistance up to about 82° C., and as generally used to bind an adherent such as a plastic (e.g., a plastic film), a wood, a metal, a paper, or a combination thereof.

15. Polyamide Adhesives

A polyamide adhesive generally comprises a thermoplastic adhesive prepared as a solvent solution, a solid hot-melt, and/or a film, and may be solidified by heat and/or pressure. A polyamide adhesive may be prepared from a condensation reaction of a diamine and/or a triamine with a dibasic acid and/or dibasic ester. In specific embodiments a polyamide adhesive comprises a homopolymer, a copolymer, an aromatic polyamide, or a combination thereof. A polyamide adhesive typically possesses water resistance, oil resistance, and flexibility; and may be used to bind an adherent such as a plastic (e.g., a plastic film), a metal, a paper, or a combination thereof. A polyamide adhesive may be used as a heat sealant.

16. Polybenzimidazole Adhesives

A polybenzimidazole adhesive typically comprises a thermosetting resin prepared from an aromatic heterocycle monomer. A polybenzimidazole adhesive may be prepared as a carrier supported film adhesive that may be solidified by heating at about 288° C. to about 344° C. under high pressure with the release of a volatile compound. A polybenzimidazole adhesive generally possesses shear strength, and thermal resistance, allowing a service temperature use up to about 260° C. in an oxidative environment, and up to about 530° C. in a non-oxidative environment. A polybenzimidazole adhesive may be used on a metal surface (e.g., steel, a metal foil).

17. Polyethylene Adhesives

A polyethylene adhesive often comprises a thermoplastic chlorosulfonated polyethylene. In some embodiments, a chlorosulfonated polyethylene adhesive function as a sealant. A chlorosulfonated polyethylene sealant may comprise an additive such as a catalyst (e.g., an oxide such as a lead oxide), a plasticizer (e.g., a dibutyl phthalate), a filler, a chlorinated paraffin, a liquid component such as a solvent (e.g., isopropyl alcohol), a colorant (e.g., pigment), or a combination thereof.

18. Polyester Adhesives

A polyester adhesive typically comprises a thermoset adhesive prepared as a paste and/or a multi-pack (e.g., a two pack) adhesive that solidifies at ambient temperatures or higher, and generally possesses heat resistance, weather resistance, moisture resistance, and chemical resistance. A polyester adhesive typically may be used to bind an adherent such as a metal (e.g., a foil), a glass, a plastic, a laminate comprising plastic, or a combination thereof. A polyester adhesive may be prepared as a hot melt adhesive. A polyester adhesive may comprise a filler. A polyester adhesive may be classified as either a saturated polyester adhesive or an unsaturated polyester adhesive. A saturated polyester adhesive typically possesses a high peel strength, and may comprise a curing agent (e.g., an isocyanate) to enhance cross-linking, and thus improved chemical resistance and thermal resistance. A saturated polyester adhesive may be used to produce a laminate comprising a plastic (e.g., polyethylene terephthalate) film. An unsaturated polyester adhesive may comprise a two pack adhesive, where one pack comprises a catalyst (e.g., a peroxide). An unsaturated polyester typically comprises a diluent (e.g., a styrene monomer), an accelerator (e.g., a cobalt naphthalene), or a combination thereof, and often solidifies at ambient conditions. An unsaturated polyester adhesive typically may be used on a glass reinforced polyester laminate; and may be used as a patching material for an automotive body part and/or a concrete flooring.

19. Polyisobutylene Adhesives

A polyisobutylene adhesive typically comprises an elastomeric adhesive prepared as a solvent solution that solidifies by solvent evaporation, and generally has good aging properties, environmental resistance, elasticity (e.g., a polyisobutylene rubber adhesive), but may be susceptible to a solvent and heat.

A polyisobutylene adhesive typically may be used to as a sealant and/or a pressure sensitive adhesive; and may be used to bind a rubber, a paper, a plastic (e.g., a plastic film), a metal (e.g., the metal foil), or a combination thereof.

20. Polysulfide Adhesives

A polysulfide adhesive typically comprises an elastomeric adhesive prepared as a liquid in a multi-pack (e.g., a two pack) adhesive, and/or a paste that solidifies at ambient conditions or higher temperatures. A polysulfide adhesive typically has oil resistance, grease resistance, solvent resistance, weather resistance, ozone resistance, and gas impermeability; and may be used to bind an adherent such as a plastic, a wood, a metal, or a combination thereof. A polysulfide sealant (e.g., a high-performance sealant) may comprise a catalyst (e.g., a manganese dioxide), an accelerator, a plasticizer (e.g., a dibutyl phthalate), an adhesion promoter (e.g., a titanate, a silane), a filler (e.g., a calcium carbonate, a vermiculite, a metal powder, a glass microsphere, a carbon sphere), a colorant (e.g., a titanium dioxide), an antioxidant (e.g. a phenyl-2-naphthylamine), a thickener and/or a thixotropic, a fatty acid, a liquid component such as a solvent (a methyl ethyl ketone, a toluene), or a combination thereof. A polysulfide sealant may be used in an aerospace application, and/or a building/construction application (e.g., a door sealant, a window sealant).

A polyimide adhesive comprises a thermosetting polyaromatic resin typically prepared as a solvent solution and/or a carrier supported film adhesive, and may be solidified at about 260° C. to about 316° C. under pressure (e.g., 10 atmospheres are more) with the release of a volatile compound. A polyimide adhesive generally possesses thermal resistance, allowing a service temperature use up to about 288° C., and may be used on a metal adherent (e.g., a steel, a metal foil).

21. Polyurethane Adhesives

A polyurethane adhesive comprises a thermosetting and/or an elastomeric adhesive that may comprise a multi-pack (e.g., a two pack) liquid adhesive, a hot melt adhesive, and/or a paste. A multi-pack polyurethane adhesive typically cures at ambient to baking condition temperatures; though an one pack polyurethane adhesive often uses air humidity to activate curing at ambient conditions. A polyurethane adhesive generally has flexibility, tensile-shear strength, an operational temperature range typically from a cryogenic temperature (e.g., about −240° C.) to up to about 122° C., but may have a susceptibility to moisture. A polyurethane adhesive may be used as a sealant. A polyurethane adhesive typically bonds to an adherent such as a plastic (e.g., a plastic film), an elastomer (e.g., a rubber), a metal (e.g., a foil), or a combination thereof. A polyurethane sealant typically comprises a filler (e.g., carbon black, a silica), an antioxidant, a UV absorber, a colorant (e.g., pigment), a flame retardant, a liquid component (e.g., a toluene), or a combination thereof; and may comprise a high performance sealant.

22. Polyvinyl Acetal Adhesives

A polyvinyl acetal (e.g., a polyvinyl butyral, a polyvinyl formal) adhesive typically comprises a thermoplastic adhesive prepared as a film adhesive, a solid, and/or a solution comprising a solvent; and solidifies typically by liquid component evaporation for a solution adhesive or heat and pressure being applied to a solid form of the adhesive. A polyvinyl acetal adhesive typically possesses chemical resistance, oil resistance, and flexibility; and typically binds an adherent such as a mica, a glass, a paper, a metal, a wood, a rubber, or a combination thereof. A polyvinyl acetal adhesive may comprise a phenolic resin to enhance binding strength.

23. Polyvinyl Acetate Adhesives

A polyvinyl acetate adhesive typically comprises a thermoplastic adhesive prepared as a film adhesive that solidifies by application of heat and/or pressure (e.g., a hot melt adhesive, a pressure sensitive adhesive), and/or a water emulsion and/or a solvent solution which solidifies by the loss of the liquid component. A polyvinyl acetate adhesive often may comprise a plasticizer, a filler, a pigment, or a combination thereof. A polyvinyl acetate adhesive typically has bond strength, acid resistance, oil resistance, grease resistance, and water resistance. A polyvinyl acetate adhesive may be used to bind an adherent such as a metal, a mica, a plastic (e.g., a plastic film), a ceramic, or a combination thereof. An emulsion polyvinyl acetate adhesive may be used to bind a porous surface (e.g., a paper, a wood).

24. Polyvinyl Alcohol Adhesive

A polyvinyl alcohol adhesive typically comprises a thermoplastic adhesive prepared as a water solution, and generally possesses oil resistance, grease resistance, fungal resistant, but may be susceptible to water. A polyvinyl alcohol adhesive often comprises a filler (e.g., a clay, a starch), a pigment, or a combination thereof. A polyvinyl alcohol adhesive may be used to bind an adherent such as a porous material (e.g., a paper, a cloth, a fiberboard).

25. Protein Adhesives

A protein adhesive (“protein glue”) comprises a protein-based (e.g., an animal protein, a soybean protein, a blood protein, a fish protein, a casein). For example, a casein adhesive typically comprises a thermoplastic adhesive prepared by precipitating a casein with an acid. A casein adhesive typically comprises a dry adhesive that may be activated by admixing with water, generally possesses solvent resistance, and may be used as a wood adhesive and/or a paper adhesive.

26. Reclaimed Rubber Adhesives

A reclaimed rubber (e.g., a reclaimed natural rubber) adhesive typically comprises an elastomeric adhesive prepared in a liquid form (e.g., an aqueous dispersion, a solvent solution) and/or a pressure sensitive adhesive (e.g., a duct tape adhesive). A reclaimed rubber adhesive typically possesses moisture and water resistance, but may be susceptible to an organic solvent. A reclaimed rubber adhesive and may be used to bond an adherent such as a rubber, a paper, a ceramic (e.g., a ceramic tile), a plastic, a fibrous material (e.g., a fabric, a wood), a leather, a metal (e.g., a painted metal), or a combination thereof.

27. Resorcinol Adhesives

A resorcinol (“resorcinol-formaldehyde adhesive”) adhesive typically comprises a thermoset adhesive prepared as a solution comprising water and an alcohol. A resorcinol adhesive often comprises a multi-pack (e.g., two pack) adhesive comprising a hardener (e.g., formaldehyde) separated in a pack. A resorcinol adhesive typically solidifies an ambient condition with moderate pressure; and generally has a service temperature up to about 177° C., solvent resistance, oil resistance, grease resistance, water resistance, and microbial resistance (e.g., mold resistance, fungus resistance). A resorcinol adhesive may be used to bind an adherent comprising a cellulose fiber (e.g., a wood surface, a paper surface, a plywood surface, a fiberboard surface), a metal, a plastic, or a combination thereof. A phenol-resorcinol formaldehyde adhesive may be prepared by combining a resorcinol base with a phenolic resin to reduce costs.

28. Silicone Adhesive

A silicone adhesive (“silicone rubber adhesive”) typically comprises an elastomeric adhesive prepared as a solvent solution that solidifies at an ambient condition to a baking temperature using a catalyst (e.g, a peroxide catalyst) with liquid component evaporation; a pressure sensitive adhesive with heat resistance and peel strength; and/or a paste adhesive and/or a sealant that cures and vulcanizes at room temperature (“room temperature vulcanizing,” “RTV”) upon contact with atmospheric moisture, with the release of either methanol and/or acetic acid as a reaction product. A silicone adhesive often comprises a polysiloxane diol (e.g., a dimethyl siloxane diol, a trifluoropropyl substituted siloxane diol, a cyanoethyl substituted siloxane diol) binder. A RTV silicone adhesive typically comprises a metallic soap (e.g., a tin octoate, a dibutyl tin dilaurate) and/or a copper catalyst curing agent. A silicone adhesive typically bind to an adherent such as a wood, a plastic, a glass, a metal, a ceramic, a silicone resin, a silicone rubber (e.g., a vulcanized silicone rubber), or a combination thereof.

A silicon sealant often comprises a vulcanization agent such as a poly-functional (e.g., an acetoxy moiety, a 2-ethylhexanoic moiety) organosilane, a catalyst (e.g., a titanate ester, a tin carboxylate), a filler (e.g., a glass microballoon, a carbon black, a fused silica, a reinforcement, an extender), a plasticizer (e.g., a silicone fluid), an adhesion promoter, a colorant (e.g., a pigment), a thickener and/or a thixotropic, a flame retardant, an anti-microbial agent (e.g., a fungicide), or a combination thereof. A silicone sealant (e.g., a caulk, a high performance sealant) may be used in a bathroom, a building, an aquarium, an electronic and/or an electrical application such as an encapsulation material, or a combination thereof.

29. Styrene Butadiene Adhesives

A styrene-butadiene adhesive typically comprises an elastomeric adhesive prepared as a latex and/or a solvent solution. A styrene-butadiene adhesive generally comprises a plasticizer (e.g., an oil), a tackifier, or a combination thereof, to improve tackiness; and typically possesses an improved aging property than a natural and/or a reclaimed rubber adhesive. A butadiene-olefin adhesive such as a styrene-butadiene adhesive may be used as a pressure sensitive adhesive. A styrene-butadiene adhesive may be used to bind an adherent such as a plastic, a laminate comprising a plastic polymer, a rubber, a wood, or a combination thereof.

30. Urea Formaldehyde Adhesives

A urea formaldehyde adhesive typically comprises a thermoset adhesive prepared as a multi-pack (e.g., a two pack) adhesive separating a hardening agent and the base until use. A urea formaldehyde adhesive may solidify an ambient conditions to a baking temperature; typically possess cold water resistance, a service temperature up to about 60° C., and may be used in a preparing a wood composite. A urea formaldehyde adhesive may be blended with a melamine formaldehyde resin, a phenol resorcinol resin, or a combination thereof, to improve heated water resistance.

31. Vinyl Vinylidene Adhesives

A vinyl vinylidene adhesive typically comprises a thermoplastic adhesive prepared as a solvent (e.g., methyl ethyl ketone) solution that cures by liquid component evaporation. A vinyl vinylidene adhesive typically has water resistance, hydrocarbon solvent resistance, grease resistance, strength, and toughness, and may be used to bind an adherent such as a porous material, a textile, a plastic, or a combination thereof.

32. Non-Polymeric Adhesives

Some adhesives are non-polymeric in nature and are contemplated for use with disclosures herein. Examples of a non-polymeric adhesive include a mucilage adhesive.

33. Mucilage Adhesives

A mucilage adhesive generally comprises a non-polymeric adhesive prepared from a seed by hot infusion, and may be used as an adhesive for paper.

### U. POLYMERIC MATERIALS' (ELASTOMERS, ADHESIVES, SEALANTS) ADDITIVES

An additive (“modifier”) used in a polymeric material (i.e., a material formulation comprising a polymer) may be incorporated (“compounded”), such as by being admixed, absorbed, etc. into the polymeric material and/or a precursor material (e.g., a monomer, a prepolymer). One or more additives may be added (e.g., sequentially added) in a stage of a preparation, processing, post cure processing, post-manufacture (e.g., during service life), or a combination thereof of such a material formulation. The additive may be selected to alter and/or confer a property in the polymeric material and/or reduce cost. Though a coating is typically a type of polymeric material, additives generally used to formulate a coating for its function and purpose are described in a separate section, and the polymeric material additives described in this section are generally selected for use in polymeric materials such as plastics, adhesives, sealants, elastomers, and such like to achieve suitable function and purpose of those material classes. Other polymeric material or other material type additives generally more typical in the formulation of a given material class (e.g., a peptidizer for an elastomer) may also described in a section for a material class.

In addition to any additives described herein, additional examples of an additive typically incorporated into a polymeric material comprises an adhesion promoter, an anti-aging additive, an anti-blocking agent, an anti-fogging agent, an antioxidant, an antiozonant, an antistatic agent, a blowing agent, a coupling agent, a cross-linking agent, a curing agent (e.g., a catalyst), a colorant, a defoamer, a degrading agent, a deodorant, a dispersant, a filler, a flame retardant, a flux (i.e., a processing flow enhancer such as a cumarone-indene resin for use in a vinyl polymer), an impact modifier, an inhibitor, an initiator, a low-profile additive, a lubricant, an antimicrobial agent, a plasticizer, a promoter, a slip agent, a processing aid, a thickening agent, a thinner, a mold release agent, a thixotrope, a nucleating agent, a stabilizer (e.g., a heat stabilizer, a light stabilizer such as an UV stabilizer also known as a “UV protector”), a surfactant, an odorant, a wetting agent, or a combination thereof. In some embodiments, an additive incorporated into a polymeric material may be the same or similar as an additive and/or other component of a surface treatment (e.g., a coating) and/or a filler described herein. For example, in certain embodiments, an extender pigment described for use in a coating, which may be referred to as a filler in the coating art, may be used in polymeric material alone or in combination with another filler described for used in a polymeric material. In such a case the extender for a coating may be suitable to confer and/or alter a desired property (e.g., a mechanical property) in a polymeric material when the size, shape, solid nature, and other properties of a coating extender and a polymeric material filler are similar or the same. In further example, an anti-insect additive described for use in a coating may be admixed and used with a polymeric material to confer insect aversion and/or pesticide activity in the polymeric material. Conversely, an additive (e.g., a lubricant) and/or other polymeric material component may be adopted for use in a coating and/or a surface treatment, such as, for example use of a lubricant normally selected for use of a polymeric material selected for use in a coating (e.g., a non-film forming coating). In other embodiments, a liquid component, such as, for example, a solvent, described for use in a coating and/or surface treatment may be selected for used as a plasticizer in a polymeric material due to suitable miscibility with a polymer of the polymeric material and/or suitable ability to undergo preparation and/or processing with a polymeric material (e.g., withstand a high temperature processing procedure). In a further example, a colorant often selected for use in a coating and/or surface treatment may be suitable in a polymeric material. These types of modifications may be done using the techniques of the art for preparation of the various compositions (e.g., a material formulation), generally with the selection of a component suitable for use in a composition in keeping with the composition's preparation conditions, purpose and function.

1. Curing Agents

A curing agent comprises a chemical that promotes curing of a polymeric composition. Examples of a curing agent comprise a catalyst, a promoter, an accelerator, an initiator, a hardener, or a combination thereof. A latent curing agent becomes active at a non-ambient condition (e.g., a baking condition temperature) and/or by contact with an activating agent. Often a catalyst may be used in the initial polymerization of a thermoplastic polymer (a Ziegler-Natta catalyst, a Philips catalyst), an elastomeric polymer, and/or a thermoset prepolymer, and in some embodiments such a catalyst may be retained as part of the polymeric material. Examples of a catalyst comprise a Ziegler-Natta catalyst (e.g., a titanium ester, an aluminum alkyl, a titanium halide, often immobilized on an inert support); a Phillips catalyst (e.g., chromium oxide); a metal alkanoate catalyst (e.g., a manganese acetate); a strong acid (a phosphoric acid, a sulfuric acid, a HCl); a latent acid catalyst (e.g., a strong acid ammonium salt typically used in an amino resin, a heat activated peroxide); an aldehyde catalyst (e.g., typically used in a phenol resin, a urea formaldehyde resin); a peroxide catalyst (e.g., a dicumyl peroxide, a methyl ethyl ketone peroxide, a benzoyl peroxide), or a combination thereof. Examples of a heat activated peroxide comprise a benzoyl peroxide, a peroxyester, or a combination thereof. A promoter comprises a catalyst enhancing chemical, and often comprises another catalyst. Examples of a promoter include a dimethylaniline, a diethylaniline, an organic cobalt salt, or a combination thereof, often used with a peroxide catalyst (e.g., a polyester catalyst). An initiator speeds up a monomers polymerization process and generally becomes part of a polymer chain, and examples comprise a free radical (e.g., a free radical enhancing the polymerization rate of a vinyl monomer), an anionic chemical, a cationic chemical, or a combination thereof. A photoinitiator often may be used in a polymerization reaction (e.g., an olefin polymerization reaction), with examples including a cationic polymerization photoinitiator such as a complex metal halide anion plus a diaryliodonium salt and/or a triarylsulfonium salt; a mixed arene cyclopentadienyl metal salt; or a combination thereof. An accelerator accelerates a curing reaction, and an example comprises a cobalt naphthanate used with a polyester resin. A hardener becomes incorporated in a polymer by chemical reaction during the curing process (e.g., an epoxy resin curing) and examples include an amine, an acid, an anhydride, or a combination thereof.

2. Cross-Linking Agents

A cross-linking agent induces a cross-link in one or more component(s) (e.g., a polymer) of a material formulation via a covalent bond, an ionic bond, or a combination thereof, though a covalent bond in more common. The cross-link may comprise a direct attachment between the component(s) and/or the cross-linking agent may form a molecular bridge between the points of attachment. An example of a cross-linking agent comprises a peroxide that decomposes at a processing temperature (e.g., a peroxide used with a saturated polymer). A diene vinyl monomer may act as a cross linking agent upon radical polymerization, with examples including an ethylene glycol dimethacrylic, a p-divinylbenzene, a N,N′-methylenebisacrylamide, or a combination thereof. A cross-linking agent in an elastomer may be known as a vulcanizing agent, and typically cross-links via a chemical reaction at a double bond in an unsaturated polymer. Often a vulcanization reaction occurs at an elevated temperature (e.g., about 170° C.). Examples of a vulcanization agent include a sulfur, a peroxide (e.g., an organic peroxide), a benzoquinone derivative, a metal oxide, a phenolic curing agent, a bismaleimide, or a combination thereof. An example of a photo-initiated cross-linking agent includes a bisarylazide. Often a vulcanization agent includes an accelerator (e.g., a benzothiazyl) and/or an initiatorlactivator (e.g., a fatty acid such as a stearic, a zinc oxide). Other examples of a cross-linking agent comprising a carboxylic acid, an ester, a hydroxyl, or a combination thereof that may comprise a substrate of an enzyme are described herein.

3. Inhibitors

An inhibitor, in the context of an uncured polymeric material, refers to chemical that retards chemical reaction that may be used to effect the working life, curing rate, storage life, or a combination thereof, of a resin typically comprising a free radical polymerizable monomer such as a vinyl monomer (e.g., a styrene monomer) and/or a polyester resin. An example of an inhibitor comprises a benzoquinone, a hydroquinone, a hydroquinone monomethyl ether, a 2,4-dimethyl-6-t-butyl-phenol, a t-butylcatechol, or a combination thereof.

4. Nucleating Agents

A nucleating agent enhances polymer crystallization and/or reduces spherulite formulation, and may alter a property such as density, impact strength, tensile properties, material clarity, the temperature of crystallization, or a combination thereof. Generally a nucleating agent may be used with a thermoplastic (e.g., a polypropylene, a PET, a polyamide), often acting during processing (e.g., injection molding). A nucleating agent may comprise a low molecular weight polyolefin, an ionomer resin, a substituted sorbitol, a sodium benzoate, a filler, a reinforcement, a pigment, or a combination thereof. An ionomer nucleating agent typically comprises a methacrylic acid-ethylene copolymer, and may be used with a PET.

5. Plasticizers

A plasticizer generally comprises a liquid component (e.g., a solvent) miscible with a material (e.g., a polymer) due to a similar solubility parameter as the material and/or may be miscible due to combination with another plasticizer, and may be non-volatile to remain with material without migration for extended periods of time during the material's normal use, and resists environmental degradation in many embodiments. A plasticizer often modifies a polymeric material's properties such as increased flexibility, reduce T_{g}, reduce Tₘₐₓ, increased toughness, decrease viscosity, increase softness, increase extensibility, decrease tensile strength, decrease modulus, or a combination thereof. In some embodiments, a plasticizer may be added prior to processing at ambient conditions and/or a slightly elevated temperature, typically by absorption and/or admixing, and aids in reducing the time and temperature a processing. Examples of a plasticizer include a phthalate ester (e.g., a dibutyl phthalate, a dicyclohexyl phthalate, a diethyl phthalate, a dihexyl phthalate, a dimethoxy phthalate, a dimethyl phthalate, a dioctyl phthalate, a diisooctyl phthalate, a diisononyl phthalate, a diphenyl phthalate), an aliphatic ester (e.g., an adipic acid diester, a fatty acid ester), a phosphoric ester (e.g., a phosphate diester, a citrate, a trimellitate, a benzoate), a biphenol derivative (e.g., an amylbiphenyl, an ortho-nitrobiphenyl, a chlorinated biphenol, a diamylbiphenyl, a benzophenone), a polyester (e.g., a polycaprolactone, a low molecular weight adipic acid polyester), an alcohol, an aromatic oil, an epoxidized ester, a hydrocarbon (e.g., a paraffin, a chlorinated paraffin), a maleic acid ester, or a combination thereof. A plasticizer may comprise a primary plasticizer, a secondary plasticizer, an extender plasticizer, or a combination thereof.

A plasticizer typically comprises a primary plasticizer having similar solubility parameter as the polymer and therefore may exude from the material during and/or after preparation in limited or no amounts. A secondary plasticizer generally has limited compatibility or may be incompatible with the polymer based on dissimilar solubility parameter, but may be added with a primary plasticizer that the secondary plasticizer has some compatibility with, to improve a plasticizers and/or a material's property such as permanence, a low-temperature property, or a combination thereof. An extender plasticizer may be used to lower-cost, and may be non-compatible or has limited compatibility with the polymer and generally exudes by itself, but may be combined with a primary and/or a secondary plasticizer to inhibit the extender plasticizer from exuding.

6. Lubricants

A type of processing aid (i.e., a material used to improve the ease of processing) comprises a lubricant that typically acts by reducing melt viscosity, particularly in a higher molecular weight polymeric material; reducing friction between a polymeric material and a machine component during processing; reducing friction between a plurality of polymeric material products; or a combination thereof. An internal lubricant generally reduces melt viscosity and/or improves melt state flow, and examples include a long chain ester, an amine wax, a montan wax ester derivative, a polymeric flow promoter, or a combination thereof. A polymeric flow promoter (e.g., ethylene-vinyl acetate copolymer, a polystyrene acrylonitrile, particularly for use with a PVC based polymeric material) lowers viscosity at an elevated processing temperature but has little or no effect on mechanical properties during a normal use temperature, and generally has similar solubility parameters as a polymer. An external lubricant (e.g., a paraffin oil, an alcohol, a ketone, a metal soap, a metal salt, a carboxylic acid such as a stearic acid) typically reduces friction between a machine component and the material; and generally has little compatibility with the polymer and may be exuded from the material; has attraction to metal usually due to a polar moiety; or a combination thereof. Often a metal soap comprises an organic acid such as stearic acid (e.g., a calcium stearate, zinc stearate). Examples of a lubricant that reduces the friction between a plurality of polymeric material products (e.g., molded articles) comprises a molybdenum disulfide, a graphite, or a combination thereof.

7. Mold Release Agents

A release agent comprises a substance to reduce adhesion between a plurality (e.g., two) of surfaces. A mold release agent may be used to promote removal of a polymeric material from a mold. Examples of a mold release agent include an internal mold release agent, an external mold release agent, or a combination thereof. Examples of a mold release agent include a metal organic acid soap (“metal organic acid salt”), a biological wax (e.g., an animal wax such as a spermaceti wax; a vegetable wax such as a carnauba wax), a hydrocarbon wax (e.g., a paraffin, a microcrystalline wax), a fatty acid (e.g., an oleic acid, a stearic acid), a fatty acid ester (e.g., a hydrogenated castor oil, a diethylene glycol monostearate), a fatty acid amide, a chlorinated fatty acids (e.g., a perfluorolauric acid), a graphite, a clay (e.g., a mica, a kaolin), a silicate (e.g., a talc), a silica, a polysaccharide (e.g., a sodium alginate), a cellulosic (e.g., a cellulose acetate, a cellophane, a flour), a polyolefin (e.g., a polypropylene, a polyethylene), a poly(vinyl alcohol), a fluoropolymer [e.g., a poly(fluoroacylate), a poly(fluoroether), a polytetrafluoroethylene], a silicone (e.g., a polyalkylmethylsiloxane, a polydimethylsiloxane), or a combination thereof. Examples of a metal used in a metal soap include a lead, a lithium, a calcium, a sodium, a potassium, a zinc, a nickel, an iron, an aluminum, a magnesium, or a combination thereof; with a stearic acid being a common organic acid in the metal soap. Examples of a fatty acid amide include an oleamide, an oleyl palmitamide, an ethylene bis-stearamide, an erucamide, or a combination thereof.

8. Slip Agents

A slip agent functions as a surface lubricant, anti-stat, mold release agent, or a combination thereof, which may aid a polymeric material's processing and/or manufacture. Examples of a slip agent include a fatty acid ester, a fatty acid amide (e.g., an oleamide, an erucamide), a wax, a metal soap (e.g., a metal stearate), or a combination thereof.

9. Diluents

A diluent may be added to a polymeric material resin to reduce resin concentration; improve ease of processing; allow increased concentration of a filler and/or a reinforcement; or a combination thereof. A diluent may be retained in the polymeric material after solidification. An adhesive and/or an epoxy resin often comprises a diluent.

10. Dispersants

A dispersant comprises a liquid component that promotes dispersal of a component of a polymeric material, typically by a solvating property.

11. Thickening Agents, Thixotropics and Thinners

A thickening agent (“thickener”) increases viscosity, under various shear conditions, of a fluid or a semifluid material such as a liquidfied polymeric material (e.g., a resin), a dispersion, a solution, or a combination thereof. Examples of a thickening agent commonly used for a resin include a talc, a diatomaceous earth, a fumed silica, and/or a carbon. A thixotropic (“thixotropic filler”) increases viscosity in a low shear condition, typically by hydrogen bond formation, but this property may be reduced at a higher sheer condition. A thixotropic may be used in a coating, an adhesive and/or a sealant to confer an anti-sage property and/or produce a material with the consistency of a gel and/or a paste. Examples of a thixotropic include an asbestos, a clay, a cellulose filler, a precipitated calcium carbonate, a fumed silica, or a combination thereof. A thinner reduces viscosity in a material formulation (e.g., a polymeric material), and typically comprises a volatile liquid component.

12. Anti-Blocking Agents

An anti-blocking agent (“flattening agent”) reduces adherence of a material formulation such as a plastic film's loose adherence to itself or another plastic film due to static electricity and/or creep. A polymeric material may comprise an antiblocking agent and/or the antiblocking agent may be added exteriorly to a surface of the material. Examples of an anti-blocking agent include a calcium carbonate, a fatty acid, a metallic salt, a plastic (e.g., a fluoroplastic, a polyvinyl alcohol, a polysiloxane), a silica (e.g., a synthetic silica), a silicate (e.g., a fine particle silicate), a talc, a wax, a paraffin, a diatomaceous earth, a coating, or a combination thereof.

13. Antistatic Agent

An antistatic agent (“antistat”) dissipates static electricity by attracting moisture to the surface of a material. An antistatic agent may be classified as an external antistatic agent or an internal antistatic agent, and typically comprises a hygroscopic substance. An external antistatic agent may be applied temporarily to the surface of a polymer material to aid in processing. An internal antistatic agent (e.g., a quaternary ammonium compound, an ethoxylated amine) may be classified either as a migratory antistatic agent that tends to migrate to the surface of a polymeric material due to poor compatibility with a polymer, or a permanent antistatic agent (e.g., a hydrophilic polymer, a conductive polymer, a conductive filler such as a metal filler, a carbon/graphite fiber, a carbon black) that may be retained in a polymeric material. Often a polymeric material comprising a conductive filler and/or a conductive reinforcement may be used as an electromagnetic shield for an electrical equipment and/or an electronic equipment (e.g., a telephone, a computer, a television set, a radio). Examples of a hydrophilic polymer include a polyethoxy polymer (e.g., a polyethylene glycol).

14. Flame Retarders

A flame retarder (“flame retardant”) reduces the flammability of a material and typically comprises a metal hydrate (e.g., an aluminum trihydrate); a phosphate (e.g., a tritolyl phosphate, a trixylyl phosphate), particularly for a PVC-based material; a halogenated compound (e.g., a chlorinated cycloaliphatic, an alkyl chlorine, an aromatic bromine such as a pentabromodiphenyl oxide, a chlorinated paraffin); an antimony oxide (e.g., an antimony pentoxide, an antimony trioxide); a borate (e.g., a barium metaborate, a zinc borate); a zinc oxide; a red phosphorus; a molybdenum compound; a titanium dioxide; or a combination thereof.

15. Colorants

A colorant generally comprises a pigment and/or an extender, which may be insoluble in the material, or a dye, which may be soluble in the material, or a combination thereof.

16. Antifoqqinq Agents

An antifogging agent prevents moisture from interfering with the view through a transparent plastic film (e.g., a PVC film), and typically comprises a fatty acid ester.

17. Odorants

An odorant often comprises a pleasant smelling compound typically used to improve the scent of a polymeric material (e.g., a thermoplastic), such as one used in a garbage bag and/or a liner for garbage can. An odorant often may be dissolved into a liquid component (e.g., a solvent), encapsulated (e.g., an encapsulating plastic pellet), or a combination thereof, for incorporation into a polymeric material often during processing.

18. Blowing Agents

A blowing agent (“foaming agent”) produces a void in a polymeric material to produce a cellular (“foamed”) polymeric material (e.g., a solid foamed polymeric material). A blowing agent may be classified as a physical blowing agent (e.g., a glass bead, a resin bead, a pressurized gas that expands under low-pressure, a volatile liquid that evaporates at a temperature being used during processing) or a chemical blowing agent, such as a chemical reaction of one or more a material's component(s) that releases a volatile chemical, a compound that decomposes into a gas, etc. Examples of a physical blowing agent comprise a compressed nitrogen gas, a volatile liquid such as a fluorinated aliphatic hydrocarbon (e.g., a chlorofluorocarbon, a chlorofluormethane), a hollow particle (e.g., a ceramic microsphere, a polymer/resin microsphere, a glass microsphere), water, a methylene chloride, or a combination thereof. An example of a chemical blowing agent comprises a foaming reaction of water with an isocyanate group of a polyurethane which produces a reaction product that decompose into CO₂; a hydrazine derivative; a tetrazole; a semicarbazide; a benzoxazine; an azo compound; a sodium bicarbonate; a dinitropentamethylene tetramine; a sodium borohydride; a polycarbonic acid; a sulfonyl hydrazide; or a combination thereof. In some embodiments, a blowing agent comprises an azodicarbonamide (e.g., a modified azodicarbonamide), a 4,4′-oxybis(benzenesulfohydrazide), a diphenylsulfone-3,3′-disulfohydrazide, a trihydrazinotriazine, a p-toliuylenesulfonyl semicarbazide, a 5-phenyltetrazole, an isatoic anhydride, or a combination thereof. A blowing agent typically may be used during injection molding to produce a foamed polymeric material (e.g., a foamed polyurethane).

19. Surfactants

A surfactant reduces the surface tension of a liquid material, and typically may be used in a polymeric material to aid in cell creation during foaming by a blowing agent. Examples of a surfactant include a cationic surfactant (e.g., a cetyl pyridinium chloride), an anionic surfactant (e.g., a sodium lauryl sulfate), a non-ionic surfactant (e.g., a polyethylene oxide), or a combination thereof.

20. Defoamers

A defoamer (“anti-foaming agent,” “antifoamer”) aids to removed a trapped gas (e.g., air) from a polymeric material, often during processing. A defoamer often has function as a surface tension depressant, a lubricant, and/or a wetting agent to promote gas release. An example of a defoamer comprises a silicone, a hydrocarbon, a fluorocarbon, a polyether, or a combination thereof.

21. Anti-Aging Additives

An anti-aging additive reduces environmental and/or other degradation caused by, for example, oxidation, (e.g., ozone chemical attack, oxygen chemical attack), light degradation, UV degradation, dehydrochlorination, or a combination thereof. Degradation that may occur due to these types of processes includes polymer chain scission, polymer chain(s) cross-linking, a polar moiety addition to a polymer chain, a discoloring chemical change, or a combination thereof. Examples of an anti-aging additive include an antioxidant, an antiozonant, a stabilizer, or a combination thereof.

An antioxidant inhibits oxidation and/or a free radical chemical reaction. Examples of an antioxidant typically used in a polymeric material include an amine antioxidant such as an aromatic amine (e.g., an arylamine); a lactone stabilizer (e.g., a benzofuranone derivative); a phenolic antioxidant (e.g., a bisphenolic such as bisphenol A, a hindered phenolic, a simple phenolic, a polyphenolic); a vitamin E; a metal salt; a thioester antioxidant (e.g., a polythiodipropionate, a thiodipropioic acid derivative); an organophosphite antioxidant [e.g., a tris-nonylphenyl phosphite, a tris(2,4,-di-tert-butylphenyl)phosphite]; a carbon black; or a combination thereof. A carbon black comprises an oxygen comprising moiety such as a phenolic, a carboxyl, a hydroxyl, a carbonyl, or a combination thereof, on the molecular surface of a carbon black particle. Examples of a hindered phenolic antioxidant include a butylated hydroxytoluene, a high molecular weight phenolic, a thiobisphenolic, or a combination thereof. In a specific facet, a phenolic antioxidant comprises a 4-methyl-2,6-di-tert-butylphenol. An amine antioxidant may be used with a polyurethane, an elastomer, or a combination thereof. A phenolic antioxidant, an organophosphite antioxidant, a thioester antioxidant, or a combination thereof, may be used with a polyolefin, a styrenic polymer, or a combination thereof. A metal deactivator (e.g., a chelator) may be used to reduce the activity of a metal ion which may act as oxidizing agent. Examples of the metal deactivator include a N,N-bis(3,5-di-tert-butyl-4-hydroxyhydrocinnamoyphydrazine; an oxalyl bis(benzylidenehydrazide); a 2,2′-oxamidobisethyl(3,5-di-tert-butyl-4-hydroxyhydrocinnamate); an oxamide (“ethanediamide”), an oxanilide (“diphenyl oxamide”), a N,N′-dibenzaloxalyldihydrazide, a benzotriazole, or a combination thereof. A peroxide decomposer (e.g., a sulfonic acid, a zinc dialkylthiophosphate, a mercaptan) may also be added to inhibit free radical production from a hydroperoxide. Examples of peroxide decomposer includes a 2-mercaptobenzothiazole; a benzothiazyl disulfide; a beta-naphthyl disulfide; a dilauryl-beta,beta-thiodipropinate; a phenothiazine; a thiol-beta-naphthol; a tris(p-nonylphenyl)phosphite; a zinc dimethyldithiocarbamate; or a combination thereof.

An antiozonant protects against ozone degradation, and may be considered herein to be a type of antioxidant. A polymer (e.g., an elastomer) comprising a double bond (e.g., an ethylenic unsaturated double bond) may be susceptible to ozone-based oxidation when under physical stress. Examples of an antiozonant include an inert polymer (e.g., an ozone resistant elastomer, a saturated polymer), a wax (e.g., a microcrystalline wax, a paraffin), a chemically reactive antiozonant, or a combination thereof. Examples of a chemically reactive antiozonant includes a nickel dithiocarbamate salt (e.g., a nickel dibutyldithiocarbamate), a thiol urea, a N-substituted urea, a substituted pyrrole, a 2,2,4-trimethyl-1,2-dihydroquinoline derivative, a p-phenylenediamine derivative such as a N,N-bis(1,4-dimethylpentyl)-p-phenylenediamine; a N,N-bis(1-ethyl-3-methylpentyl)-p-phenylenediamine; a N,N-bis(1-methylheptyl)-p-phenylenediamine; a N-cyclohexyl-N′-phenyl-p-phenylenediamine; a N-(1,3-dimenthylbutyl)-N′-phenyl-p-phenylenediamine; a N-isopropyl-N′-phenyl-p-phenylenediamine; a N-(1-methylheptyl)-N′-phenyl-p-phenylenediamine; a N-(1-methylpentyl)-N′-phenyl-p-phenylenediamine; a N,N′-me\ixed diaryl-p-phenylenediamine; a N,N-diphenyl-p-phenylenediamine; a N,N′-di-2-naphthyl-p-phenylenediamine; a N,N-dicyclohexyl-p-phenylenediamine; or a combination thereof. A wax (e.g., a surface treatment wax) may retard penetration of ozone, and examples a wax includes a paraffin, a microcrystalline wax, or a combination thereof.

A stabilizer comprises a chemical used to maintain a property (e.g., a physical property, a chemical property) during processing and/or service life of a polymeric material. Examples of a stabilizer include a heat stabilizer, a light stabilizer (e.g., UV stabilizer), or a combination thereof. A heat stabilizer reduces thermal degradation of a polymeric material. A heat stabilize may be used with a polymer comprising chlorine to reduce dehydrochlorinization and/or reacts with a product of dehydrochlorinization. Examples of a heat stabilizer include a metal salt (e.g., a zinc salt, a zinc-calcium salt, tin salt a barium salt, a barium-zinc salt; with the salt often comprising an organic acid salt such as a maleic acid, a phthalic acid, etc); a lead compound (e.g., a red lead oxide); an antimony mercaptide; an organo-tin compound, which may be used to retard dehydrochlorination; an antioxidant (e.g., a bisphenolic such as bisphenol A) which may be used to retard dehydrochlorination; an epoxy compound; a polyol; an organophosphite; a beta-diketone, which may be used to react with a product (e.g., HCl) of dehydrochlorination; and acid receptor (e.g., a barium carbonate, a magnesium oxide), or a combination thereof.

Photodegradation may occur, for example, as UV light absorption by a material to produce a free radical, often by a breaking a double bond in the polymer followed by peroxide formation. Examples of a UV stabilizer include a UV absorber and/or a UV screener (e.g., a phenyl ester, a titanium dioxide, a zinc oxide, a carbon black, a benzophenone, a diphenylacrylic, a salicylate, an aryl ester such as a resorcinol monobenzoate, an oxanidide); a quenching agent (e.g., a hindered anime, a nickel organic complex) of a radicalized and/or a chemically activated molecule (e.g., a radicalized polymer); a metal salt (e.g., a manganese salt, a copper salt); a peroxide decomposer; or a combination thereof. An examples of a phenyl ester includes a 3,5-di-t-butyl-4-hydroxybenzoic acid N-hexadecyl ester. Examples of a benzophenone include a benzotriazole [e.g., a 2-(o-hydroxyphenyl)benzotriazole], a 2,4-dihydroxy-4-n-dodecycloxybenzophenone, a 2-hydroxy-4-methoxybenzophenone, a 2-hydroxy-4-n-octoxybenzophenone, an o-hydroxybenzophenone, a 2-(o-hydroxyphenyl)benzotriazole], or a combination thereof. Examples of a benzotriazole include a 2-(3′,5′-di-tert-butyl-2′-hydroxyphenyl)-5-chlorobenzotriazole; a 2-(2-hydroxy-3′-5′-di-tart amyl phenyl)benzotriazole; a 2,2-(2-hydroxy-5-tert-octylphenyl)benzotriazole; a 2-(3′-tert-butyl-2-hydroxy-5-methylphenyl)-5-chlorobenzotriazole; or a combination thereof. Examples of a diphenylacrylate include a 2-ethylhexyl-2-cyano-3,3-diphenyl acrylate; an ethyl-2-cyano-3,3-diphenyl acrylate; or a combination thereof. Examples of a hindered amine light stabilizer (“HALS”) include derivatives of 2,2,6,6-tetramethyl-4-piperidinyl such as a bis(2,2,6,6-tetramethyl-4-piperidinyl) sebacate; a methyl(2,2,6,6-tetramethyl-4-piperidinyl) sebacate; a N,N-bis(2,2,6,6-tetramethyl-4-piperidinyl)-1,6-hexane diamine polymer; or a combination thereof. Examples of a nickel organic complex include a 2,2′-thiobis(4-octylphenolato)-n-butylamine nickel; a nickel dibutyldithiocarbamate; or a combination thereof.

22. Degrading Agents

A degrading agent enhances biodegradation of a material. Examples of the degrading agent include a biodegradable polymer such as a starch to foster microbial growth upon and within a material; and/or a photodegradation enhancing material such as a UV absorber.

23. Anti-Microbial Agents

An anti-microbial agent typically comprises a biocide (e.g., a fungicide, a bactericide, a herbicide a mildewcide, an algaecide, a viricide, a germicide, a microbiocide, a slimicide) and/or a biostatic (e.g., a fungistatic, a bacteristatic, a mildewstatic, an algaestatic, a viristatic, a herbistatic, a germistatic, a microbiostatic, a slimistatic) to inhibit the growth of an organism such as a bacteria, a fungi, a mildew, an algae, a virus, a microorganism, or a combination thereof, on and/or within a material formulation. An anti-microbial agent within a polymeric material typically diffuses and/or travels to the surface of the polymeric material during normal service life to provide a more continuous activity at the surface in reducing microbial grow. Often an anti-microbial agent comprises a carrier such as a liquid component (e.g., a solvent, a plasticizer), a resin, or a combination thereof. Specific examples of a carrier typically used as an anti-microbial agent carrier includes plasticizer (e.g., a diisodecyl phthalate, an epoxidized soybean oil), an oil, or a combination thereof. Examples of an anti-microbial agent commonly used in a polymeric material includes 2-n-octy-4-ixothiazonin-3-1; 10,10-oxybisphenoxarsine (“OBPA”); zinc 2-pyrodinethanol-1-oxide (“zinc-omadine”), trichlorophenoloxyphenol (“trislosan”), or a combination thereof, though a preservative used in a coating as well as an anti-microbial peptide are contemplated for use as an anti-microbial agent in a polymeric material, and such an anti-microbial agent may be used either alone or in combination with another anti-microbial agent in any composition, article, method, machine, etc. described herein in light of the present disclosures. An antimicrobial agent generally comprises about 0.000001% to about 1% of a polymeric material, and about 2% to about 10% of and anti-microbial agent and a carrier mixture, respectively, though given the inclusion of a biomolecular composition as part of a polymeric material and other compositions described herein, the content of an antimicrobial agent may be increased from about 0.000001% to about 10% or more. An antimicrobial agent often acts as a deodorant by reducing the growth of odor producing microorganism, particularly in a fiber (e.g., a textile) and/or a polymeric film application for packaging of food and/or trash.

24. Adhesion Promoters

An adhesion promoter typically comprises a liquid that forms a molecular layer between an adhesive and an adherent; a polymer and a filler and/or a reinforcement; or a combination thereof, to improve adhesion between the materials. Examples of an adhesion promoter include a benzotriazole, a chrome complex, a cobalt compound, a 1,2-diketone, a silane, a titanate, a zirconate (e.g., a zirconium propionate), or a combination thereof. Typically an adhesion promoter improves the adhesion between an organic (e.g., an organic polymer) and an inorganic material (e.g., a glass fiber).

A coupling agent comprises an adhesion promoter comprising an inorganic moiety and an organic moiety to promote adhesion between an inorganic material and an organic material. For example, a silane may comprise an amino moiety, an epoxy moiety, a methoxy moiety, a methacrylate moiety, a vinyl moiety, or a combination thereof to promote a covalent bond linking a resin (e.g., an acrylic, a phenolic, a polyamide, a polyester, a PVC, an EPDM, a furan) and a filler and/or a reinforcement (e.g., a clay, a mica, a sand, a Wollastanite, a calcium sulfate, an alumina, an alumina trihydrate, a silica carbide, a talc). A titanate and/or a zirconate comprise a moiety (e.g., a carboxylic acid) that promotes hydrogen bonding to a polyolefin. Examples of a coupling agent and an associated chemical moiety include a 3-(N-styrylmethyl-2-amino-ethylamino)propyltrimethoxysilane hydrochloride comprising a cationic styryl; a 3-aminopropyltriethoxysilane comprising a primary amine; a 3-glycidoxypropyltrimethoxysilane comprising an epoxy; a 3-mercaptopropyltrimethoxysilane comprising a mercapto; a 3-methacryloxypropyltrimethoxysilane comprising a methacrylate; a beta-(3,4-epoxycyclohexyl)ethyltrimethoxysilane comprising a cycloaliphatic epoxide; a chloropropyltrmethoxysilane comprising a chloropropyl; a N-2-aminoethyl-3-aminopropyltrimethoxysilane comprising a diamine; a silane that may comprise various moiety(s); a titanate [“tris(methacryl)isopropyl titanate”] comprising a methacrylate; a vinyltrimethoxysilane comprising a vinyl moiety; a volan comprising a chrome complex; a zirconate comprising a carboxylic acid; or a combination thereof.

25. Impact Modifiers

An impact modifier enhances the impact strength of a material. Generally an impact modifier comprises an elastomer and/or a more elastic polymer relative to a more rigid polymer in a polymeric material. An impact modifier may be semi-compatible or compatible (e.g., semimiscible, miscible) with the more rigid polymer. For example, an olefinic thermoplastic (e.g., a polyethylene, a polypropylene, a polybutylene) may comprise an olefinic elastomer (e.g., a thermoplastic elastomer) as an impact modifier. A blend of a polymer and an impact modifier polymer generally produces a two-phase polymeric material. The impact strength of the polymeric material may be improved at room temperature or lower temperatures, though the formulation of the polymeric material may be so designed to improve impact strength at an elevated temperature. Examples of a polymeric impact modifier include an ethylene propylene rubber; an ethylene propylene diene monomer; a SAN-g-EPDM; a maleated EPDM; a maleated polypropylene; a maleated polyethylene; a chlorinated polyethylene; a methylacrylatelacrylonitrile-butadiene-styrene; a methylacrylate-butadiene-styrene; a polymethylmethacrylate; a polyurethane; a styrene butadiene rubber; an acrylonitrile-butadiene-styrene; an ethylene-vinyl-acetate; or a combination thereof.

26. Low-Profile Additives

A low-profile additive refers to an elastomeric and/or a thermoplastic polymer blended/compounded with a material formulation such as a composite (e.g., a polyester composite comprising a glass reinforcement), a reinforced polymeric material, and/or a molding compound (e.g., a bulk molding compound, a sheet molding compound) to enhance one or more surface properties such as appearance, cracks, surface waviness, dimensional shrinkage, etc. Often a low-profile attitude may be used with a polyester (e.g., an unsaturated polyester). Examples of an elastomeric low profile additive include a styrene-butadiene-styrene and/or a butadiene-styrene. Examples of a thermoplastic polymer typically used as a low-profile additive includes a polyethylene, a polyamide, a polystyrene, an acrylic (e.g., a polymethylmethacrylate), a polyvinyl acetate, or a combination thereof. Often about 0.0000001 to about 15 weight percent of an elastomer may be used, while about 0.0000001% to about 50% of a thermoplastic may be used, in a low-profile polymeric material. A reduced content (e.g., up to about 30% for a thermoplastic) of a low profile additive may be known as a low shrink additive, and such a polymeric blend comprising a reduced amount of a thermoplastic and/or an elastomer may be known as a low shrink resin.

27. Fillers

A filler for use in a polymeric material comprises a solid (e.g., an insoluble) additive incorporated into polymeric material (e.g., a reinforced polymeric material, a composite). In some embodiments, a filler may be used to alter a property such as enhance hardness, enhance creep resistance, increase impact resistance, increase the heat deflection temperature, alter (e.g., increase) density of the material, reduce the shrinkage of the material, alter electrical conductivity, alter thermal conductivity, or a combination thereof.

In specific aspects, a biomolecular composition (e.g., a cell based particulate material) may be used as a filler (e.g., a reinforcement). In some facets, such a biomolecular composition based filler may be used to promote biodegradation in a material formulation (e.g., a biodegradable surface treatment, a biodegradable polymeric material, a biodegradable filler), and may be combined with one or more component(s) of a material formulation selected as also being biodegradable (e.g., a biodegradable polymer). In other embodiments, a filler/reinforcement may bond (e.g., covalently attach, ionically attacy) to a component (e.g., a polymer) of a material formulation without an agent such as a coupling agent, a crosslinking agent, and/or the like.

A filler may comprise electrically conducting and/or thermally conducting filler, to modify a polymeric material's insulation against heat and/or electrical conduction. For example, an electrically conducting filler may confer an electromagnetic interference shielding property and/or an antistatic property to produce a shielding compound and/or to transmit a current. An electrically conducting filler may be used in an electrical and/or an electronic application such as an electrode, a keyboard, a housing, a cabinet, or a combination thereof. Examples of a conductive filler include a silica, an aluminum nitride, a boron, an aluminum filler, a vapor grown fiber, a diamond fiber, an ultrahigh thermal conductivity pitch fiber, or a combination thereof, for thermal conduction; as well as a carbon black, a carbon fiber (e.g., a fabric, a mat); a metal filler (e.g., an aluminum filler such as an aluminum flake) for thermal and/or electrical conduction; or a combination thereof. Examples of a metal filler include a metal powder; a metal fiber; a metal coated microsphere; a metal coated fiber (e.g., an organic fiber coated with a metal), or a combination thereof. In some embodiments a filler comprises a magnetic and/or a ferrous filler such as a ferrite (e.g., an iron oxide, a lead ferrite, a strontium ferrite, a barium ferrite), which may be used to produce a polymeric material comprising a rigid magnet and/or a flexible magnet.

In some cases a filler (e.g., carbon black) may act as a pigment, a UV protector, or a combination thereof. In some embodiments a filler may comprise a particular material; a fibrous filler such as a synthetic fiber (e.g., a polyamide fiber), a natural fiber glass (e.g., a cotton), a carbon/graphite fiber, or a ceramic fiber (e.g., a metal oxide fiber, a silicone whisker); or a combination thereof.

In other embodiments, a filler may comprise an organic filler (e.g., a cellulosic filler, a lignin filler, a synthetic organic fiber, an animal filler, a carbon filler, a reclaimed filler), an inorganic filler, or a combination thereof. Examples of a cellulosic filler includes a flour (e.g., a wood flour, a shell flour such as a cherry stone flower, a walnut shell flower, a pecan shell flower), a fiber (e.g., an alpha cellulose fiber, a rayon fiber, a jute fiber, a hemp fiber, a sisal fiber, a kapok fiber, a coir fiber, a ramie fiber, an abaca fiber, a pulp preform, a cotton fiber/flock, a textile byproduct, a paper), a chip, a corncob, a grain hull (e.g., a rice hull), a diced resin board, or a combination thereof. Examples of an organic paper include a kraft paper, a chopped paper, a crepe paper, or a combination thereof. A cellulosic filler may be prepared from a plant source. Examples of a lignin filler includes a processed lignin, a ground bark, or a combination thereof. Examples of an organic synthetic fiber include a cellulosic thermoplastic fiber, an acrylic fiber, a polyamide fiber, an aramid fiber, a fluoropolymer, a polyester fiber, a polyethylene fiber, a polypropylene fiber, a polyurethane fiber, another synthetic polymeric fiber described herein, or a combination thereof, with all of these examples of an organic synthetic fiber also being examples of a polymeric fiber. Examples of an animal filler include an animal fiber (e.g., a llama hair, a goat hair, a camel hair, a cashmere, a mohair, an alpaca, a vicuna wool, a silk fiber). Examples of a carbon filler includes a graphite filament, a graphite whisker, a ground petroleum coke, a carbon black (e.g., a furnace black, a channel black), or a combination thereof. Examples of a reclaimed filler include a reclaimed rubber (e.g., a nitrile rubber), a thermoplastic filler, a macerated cord, a macerated fabric, or a combination thereof.

Examples of an inorganic filler include and an aluminum trihydrate, a barium ferrite, a barite filler (e.g., a lead sulfate, a barium sulfate, a strontium sulfate, a barium chromate sulfate), a boron filler (e.g., a boron fiber, a boron filament, a boron whisker), a calcium carbonate filler (e.g., a precipitated calcium carbonate, a ground calcium carbonate, a whiting/chalk, a limestone), a glass filler, a metal filler (e.g., a metal, a metal oxide, a fiber, a filament, a whisker), an inorganic polymeric filler, a silica filler (e.g., a silica mineral, a silica synthetic filler), a silicate (e.g., a silicate mineral, a silicate synthetic filler), or a combination thereof. Examples of a glass filler include a glass sphere (e.g., a solid glass sphere, a hollow glass sphere), a glass flake, a glass fiber (e.g., a fabric, a filament, a mat, a milled fiber, a roving, a woven roving, a yarn), or a combination thereof. Examples of a metal (e.g., a metal alloy) often used as a filler (e.g., a fiber, a filament), a metallized surface deposit, and/or an adherent for attachment of an adhesive, a sealant, a surface treatment, or a combination thereof, include an aluminum, a beryllium, a copper (e.g., a bronze, a brass), a cadmium, a chromium, a gold, an iron (e.g., a stainless steel), a germanium, a lead, a magnesium, a molybdenum, a nickel (e.g., a nickel phosphorus alloy), a silver, a tin, a titanium, a thorium, a tungsten, a zinc, a palladium, a platinum, a zirconium, a uranium, or a combination thereof. Examples of a metal oxide filler include a titanium oxide (e.g., a titanium dioxide), a zinc oxide, a magnesium oxide, an aluminum oxide, or a combination thereof. Examples of a metal whisker include a metal oxide (e.g., a magnesium oxide, an aluminum oxide, a zirconium oxide, a beryllium oxide, a thorium oxide), a metal nitride (e.g., an aluminum nitride), a metal carbide, or a combination thereof. Examples of a silica mineral filler include a diatomaceous earth, a quartz, a sand, a tripoli, or a combination thereof. Examples of a synthetic silica filler include a silica aerogel, a ground silica, a pyrogenic silica, a wet process silica, a silicon whisker (e.g., a silicon nitride, a silicon carbide), or a combination thereof. Examples of a silicate mineral include an actinolite (e.g., a kaolinite/china clay, a mica, a talc, a Wollastanite), an asbestos, an amosite, an anthophyllite, a crocidolite, a chrysolite, a tremollite, or a combination thereof. Examples of a kaolinite include a surface treated kaolin, a calcined kaolin, an air floated kaolin, or a combination thereof.

An inert filler (“inert,” “extender filler,” “extender”) typically may be used to reduce the cost of a polymeric material but may affect other properties such as reduce shrinkage, increased heat deflection temperature, alter (e.g., increase) composition density, increased hardness, or a combination thereof. An example of an inert filler includes a china clay (“kaolin”), a sand/Quartz powder, a calcium carbonate (e.g., limestone), a glass microsphere (e.g., a solid glass microsphere, a hollow glass microsphere), a mica, a wollastonite, a silica, a barium sulfate, a metal powder (e.g., a metal oxide), a carbon black, a talc, a fiber (e.g., a cellulose fiber, a cotton fiber, a wood flour, a carbon fiber, a fiberglass), a whiting, or a combination thereof. A microsphere may be between about 4 μm to about 5000 μm in diameter; though a hollow microsphere are generally up to about 200 μm in diameter.

A reinforcing filler (“reinforcement,” “reinforcing material”) may be used to increase a mechanical property such as modulus, tensile strength, compressive strength, shear strength, stiffness, and/or impact strength; increase the heat deflection temperature; improve creep behavior; reduce shrinkage; or a combination thereof. In many embodiments, a reinforcing filler may occupy a void in a polymer matrix, form a chemical bond with a component of the polymeric material (e.g., a polymer), or a combination thereof. A smaller filler particle size tends to enhance mechanical properties, while a larger particle size may negatively affect such a property. Examples of a reinforcing filler comprises a reinforcing lamellar/plate shaped filler (e.g., a graphite, a talc, a kaolin, a mica), a reinforcing spherical filler, a reinforcing mineral filler, a reinforcing cellulose filler, a reinforcing glass particulate filler, a reinforcing nanofiller, a reinforcing fibrous (“fiber,” “filament,” “fibre”) filler (e.g., a cellulosic fiber; a synthetic fiber; an asbestos fiber; a carbon fiber; a whisker such as a crystal fiber, a crystal filament; a glass fiber; a wollastonite; a nanofiber), or a combination thereof.

Examples of reinforcing spherical filler include a metallic oxide, a calcium carbonate, a hollow glass sphere, a solid glass sphere, a silica, a sand, a quartz powder, a carbon black, or a combination thereof. Examples of a reinforcing mineral filler include a crystalline silica, a calcium sulfate (e.g., an anhydrous calcium sulfate, a dehydrated calcium sulfate), a fused silica, a quartz, a treated mica, a vermiculite, a boron nitride particle, a silver particle, an aluminum nitride particle, an alumina particle, an iron/steel particle, a feldspar, a nepheline syenite, a talc, a Wollastanite, a sapphire, a diamond, or a combination thereof. Examples of a reinforcing cellulose filler includes a wood flour. Examples of a reinforcing glass particulate filler includes a glass bead, a glass flake, or a combination thereof. In some cases a reinforcing filler comprises a nanofiller, which possesses an extremely high surface area ratio such as a particulate (e.g., a clay platelet, a fullerine) that has a thickness of about 0.1 nm to about 10 nm, and may achieve desired properties with about 10 fold less (e.g., about 0.1% to about 8% reinforcement content) reinforcement material than a typical filler (e.g., a mineral filler).

A reinforcement often comprises a fiber. A reinforcing fiber has a length to diameter ratio of about 10:1 or greater, and typically has a diameter up to about 10 mm, and a length greater than about 100 mm. In some cases a reinforcement fiber comprises a nanofiber (e.g., a carbon nanotube), which comprises an extremely high surface area ratio relative to other fibers, and typically has a diameter of about 0.1 nm to about 10 nm, and may achieve desired properties with about 10 fold less (e.g., about 0.1% to about 8%) reinforcement material than a typical fiber reinforcement.

A fiber typically comprises a plurality of individual fiber units prepared into a strand, while a plurality of individual strand units may be prepared into a yarn (e.g., a plied yarn, a twisted yarn), and a plurality of individual yarn units woven into a fabric, etc. Thus, a fiber may be in the form of separate strand units (e.g., a chopped strand, a milled fiber, a short “discontinuous” fiber, a long “continuous” fiber, a staple), a whisker (i.e., an elongated crystal), a twisted yarn, a plied yarn, a tape, a braid, a tow, a fabric (e.g., a unidirectional fabric, a knitted fabric, a chopped fabric, a linen, a scrim), a ribbon, a flock (e.g., a chopped flock), a roving (e.g., a spun roving), a woven roving, a mat (e.g., a chopped strand mat, a continuous strand mat, a combination woven roving mat, a surfacing mat), a three-dimensional reinforcement (“preformed shape”; i.e. a yarn and/or braided strand prepared in a continuous, bulky shape), a paper, or a combination thereof.

Examples of materials used for a fiber reinforcement include a synthetic fiber, an organic fiber, an inorganic fiber, a nanofiber, or a combination thereof. Examples of a synthetic fiber include a glass fiber (“fiberglass”), an acrylic fiber, polyethylene terephthalate fiber, a boron fiber, a carbon/graphite fiber, a diamond fiber, a polyaramide fiber (“aramide fiber”; e.g., a Kevlar fiber, a nylon), an asbestos fiber, a polypropylene fiber, a polyethylene fiber, a poly(p-phenylene-2,6-benzobisoxazole) (“PBO”) fiber, a rubber fiber, a vapor-grown fiber, or a combination thereof.

A glass used in a reinforcement (e.g., a fiber, a filler) may include an A-glass, D-glass, a C-glass, a D-glass, an E-glass, a G-glass, a H-glass, a K-glass, a S-glass, a S2-glass, an E-glass, a K-glass, a R-glass, a Te-glass, a high silica Zentron glass, or a combination thereof. A carbon/graphite fiber may be prepared from a precursor fiber [e.g., a polyacrylonitrile (“PAM”) fiber, a rayon fiber, a petroleum pitch fiber, a coal tar pitch fiber, an organic fiber], with a higher degree of graphitization correlated with improved thermal conductivity, higher modulus, and/or electrical conductivity. Examples of a carbon/graphite fiber include a standard modulus PAN fiber, an intermediate modulus PAN fiber, a ultrahigh modulus (i.e. a moduli greater than about 70 GPa) PAN fiber, an ultrahigh thermal conductivity carbon (e.g., pitch) fiber, and/or an ultrahigh modulus pitch fiber. Examples of an organic fiber include a cellulosic fiber (e.g., a paper, a wood sheet), a cotton fiber (e.g., a flock, a linen), a wool fiber, a flax fiber (e.g., a flock, a linen), or a combination thereof. Examples of an inorganic fiber include a metal fiber (e.g., a wire, a metal wool), a ceramic fiber (e.g., a silicon carbide fiber, a silicon nitride fiber, a silica fiber, an alumina fiber, an alumina silica fiber), or a combination thereof.

A reinforcement (e.g., a fiber) may be coated with a finish/sizing to improve ease of handling, enhance bonding between the reinforcement and the polymer, protect the reinforcement from the polymeric (e.g., a composite) material's component(s), protect the reinforcement from environmental damage, or a combination thereof. The sizing/finish (e.g., a wax, a starch) for a reinforcement for use in a thermosetting resin may be less suitable for use in a thermoplastic resin.

### V. TEXTILE FINISHES

A textile finish refers to a surface treatment used upon a fiber (e.g., a fabric) to confer and/or alter a property such as watery repellency, an antistatic property, a type of surface feel to the touch (e.g., softness), ease of processing, adhesion to a resin, or a combination thereof. Examples of a textile finish includes a lubricant, an anti-slip agent (e.g., a rosin, a cellulosic polymer), a softener, antistatic agent, a plasticizer, a water repellent (e.g., a wax such as a paraffin, a silicone), a crease/wrinkle resistance property resin (e.g., a melamine formaldehyde, a urea formaldehyde, a cyclic urea) thought to induce cellulose polymer chain cross-linking, an adhesive promoter for a fiber reinforcement, or a combination thereof.

### W. ADDITIONAL ENZYME USES

In certain embodiments, the compositions, articles, methods, etc. that comprise a biomolecular composition with organophosphorus compound degradation ability may have use in three primary markets that may benefit from a susceptible surface covered with a self-decontaminating coating: domestic military, friendly foreign military/civilian, and domestic civilian. For military use, a self-decontaminating coating has utility on a surface of a vehicle, a trailer, a barrack, a decontamination shelter, a piece of equipment (e.g., a piece of electronic equipment) or a combination thereof.

A biomolecular composition may have dual military and/or civilian use in a method for facilitating the disposal of a chemical waste, including but not limited to, a CWA, a pesticide or a combination thereof. A particular dual use embodiment includes coating a surface that may be in a facility where there may be an unacceptable delay to the use of a piece of equipment, a space (e.g., a room, a command center, a computer center), a vehicle (e.g., a public transportation vehicle, an emergency vehicle) or a combination thereof if the facility was subjected to and/or suspected of exposure to, a dangerous chemical (e.g., a nerve agent). In some aspects, the piece of equipment, the space, and/or the vehicle may be used by a military personnel, an emergency personnel or a combination thereof. A facility may be contacted with a chemical from a chemical weapon attack (e.g., a CWA gas attack), an accidental release of a chemical, or a combination thereof. Examples of such facilities include a control room at a military base, an airport, a nuclear power plant, a hospital, or a combination thereof. A facility (i.e., a space, a vehicle, a piece of equipment) that may be subject to exposure to a chemical (e.g., a nerve agent) may be coated with the disclosed compositions and then be detoxified and safe after contact with the chemical.

Civilian applications contemplated include a coating of a surface in contact with air, such as for example, a ventilation intake and/or an air filter, as well as a surface (e.g., an interior surface, an exterior surface) comprised in a hospital clean room, a community safe room, a control room for a nuclear plant, a control room for a chemical plant, a control room for a power plant, a control room for a water plant, a government building, an industrial building, a facility for public transportation (e.g., a train, a subway, a plane, an airport), and a surface of an equipment by a first responder, or any combination of the forgoing.

For each formulation of a coating and a biomolecular composition, enzymatic decontamination parameters based on chemical (e.g., CWA simulant) degradation assessment may be established in a range of exterior weathering conditions. If a specific formulation of enzyme composition in a coating remains active after exposure to exterior weathering conditions, there may be a significant utility for using the bioactive painted surfaces in exterior and field application. For example, in some embodiments a biomolecular composition incorporated in standard formulations of water-based and/or latex-based paint may result in reduced to no changes in the durability of the paint based on standard exterior weathering conditions. In a general aspect, a weathering study may indicate a value to reformulate a composition to improve a particular property (e.g., enhance biomolecular composition stability). In this aspect, standard methods known in the art (e.g., encapsulation), may be used to increase stability and re-test the resulting formulation. Application of such methods may be used to modify various formulations to produce a composition with one or more properties suited for a particular application, as described herein and as understood in the art in light of the present disclosures.

### X. ADDITIONAL ENZYME USES—COMBINATIONS OF DECONTAMINATION COMPOSITIONS AND METHODS

In certain embodiments, a composition, article, method etc. that possesses an organophosphorus degradation ability may be combined with another composition method for decontamination (e.g., detoxification, degradation) of a chemical. In some aspects, the additional composition or method comprises one for decontamination of a pesticide or chemical warfare agent. Such additional compositions and methods (e.g., see Yang, Y. C. et al., 1992), and may be applied prior, during and/or after application of a composition and/or method. In particularly additional embodiments, such a combination of a composition and/or method disclosed herein with a traditional composition and/or method produces greater decontamination than that achieved without such a combination.

Additional compositions that are contemplated include, but are not limited to, a caustic agent; a decontaminating foam (e.g., Sandia, Decon Green); an application of intensive heat and carbon dioxide for a sustained period; an incorporation of a material into a coating that, when exposed to sustained high levels of UV light, degrades a chemical; a chemical agent resistant coating; or a combination thereof. Examples of a caustic agent include a bleaching agent, DS2, or a combination thereof.

As used herein, a “caustic agent” comprises a composition capable of destroying usually via a chemical reaction, a material, unfortunately including animal tissue such as skin. Thus, application of a caustic agent may be accompanied by the wearing of protective gear for those not contaminated or suspected of being contaminated. Certain caustic agents, such as for example, a bleaching agent and/or decontamination solution 2 (“DS2”), have specifically been formulated and/or used to decontaminate chemical warfare agents. Both G agents and VX may be decontaminated with these caustic agents. As used herein, a “bleaching agent” refers to a reactive chemical compound capable breaking a double bond in another chemical compound, which may be a useful property for degrading a chemical (e.g., a toxic chemical). Examples of a bleaching agent include a bleach powder, a bleach solution, or a combination thereof. A bleach powder may comprise, but is not limited to, Ca(OCl)Cl and Ca(OCl)₂ (“high test hypochlorite,” “HTH”); Ca(OCl)₂ and CaO (“super tropical bleach,” “STB”); Ca(OCl)₂ and MgO (“Dutch powder”); or a combination thereof. A bleach solution may comprise, but is not limited to, NaOCl (“bleach”), usually 2% to 6% wt in water; a HTH slurry, usually 7% HTH wt in water; a STB slurry, usually 7% to 70% wt in water; activated solution of hypochlorite (“ASH”), usually 0.5% Ca(OCl)₂ and 0.5% sodium dihydrogen phosphate buffer and 0.05% detergent in water; self-limited activated solution of hypochlorite (“SLASH”), usually 0.5% Ca(OCl)₂ and 1.0% sodium citrate and 0.2% citrate acid and 0.05% detergent in water; or a combination thereof. Bleach, Dutch powder, ASH and SLASH are generally applied to skin and equipment for decontamination, while HTH and STB are generally applied to equipment and terrain for decontamination. VX may be decontaminated at an acid pH, wherein it may be more soluble (Yang, Y. C. et al., 1992).

DS2 was developed to function at various temperatures (i.e., −25° C. to 52° C.), particularly those below the freezing point of many aqueous compositions. It usually comprises 70% diethylenetriamine (H₂NCH₂CH₂NHCH₂CH₂NH₂), 28% ethylene glycol monomethyl ether (CH₃OCH₂CH₂OH), and 2% sodium hydroxide (NaOH). DS2 may be noncorrosive to many metals, but may be damaging to many paints, leathers, rubber materials, plastics and skin. Contact with a paint may be limited to 30 minutes or less. An aqueous rinse may be used to remove DS2, and exposure to air and/or water degrades DS2 (Yang, Y. C. et al., 1992).

Various other decontamination compositions and methods are known in the art. Examples of a decontaminating foam include Sandia, Decon Green, or a combination thereof. Examples of an incorporation of a material include incorporation of TiO₂ and porphyrins into acetonitrile coatings that, when exposed to a sustained high level of UV light in an oxygen environment (e.g., air), degrade a chemical agent (e.g., mustard). Addition of water to the acetonitrile coating comprising TiO₂ and porphyrins may aid the degradation of VX to non-toxic compounds (Buchanan, J. H. et al., 1989; Fox, M. A., 1983). Additionally, CARCs have been developed to withstand repeated decontamination efforts. Decontamination compositions are often prepared and packaged in equipment for easy of handling. Such an equipment packages include, but are not limited to, kits (e.g., a towelette package), and delivery apparatus (e.g., a sprayer). Examples of specific decontamination equipment packages that may be used in combination with a composition, article, method, etc. include an ABC-M11 portable decontamination apparatus, which comprises DS2, a devise for spraying DS2, and a vehicle mounting bracket; an ABC-M12A1 power-driven, skid-mounted decontamination apparatus, which comprises a personnel shower unit, a pump, a tank, a M2 water heater, and delivers water, foam, DS2, STB, and/or deicing liquid; a M258A1 personal decontamination kit, which comprises towelettes soaked with a decontamination solution (i.e., 72% ethanol, 10% phenol, 5% NaOH, 0.2% ammonia, and 12% water), ampules of a decontaminating solution (5% ZnCl₂, 45% ethanol, 50% water) for adding to a towlette soaked with chloramines-B (PhS(O)₂NCINa), packing foil, and a plastic carrying case; a M280 individual equipment decontamination kit, which comprises twenty fold the contents of the M258A1Kit; a M291 skin decontamination kit, which comprises six XE-555 resin (i.e., styrene/divinyl benzene copolymer, a strong acid cation-exchange resin and a strong base anion-exchange resin for absorption and chemical detoxification) filled fiber pads packaged in foil; a M13 portable decontamination apparatus, which comprises DS2, a container and an equipment/vehicle mount, and capable of dispensing DS2; a M17 lightweight, transportable decontamination apparatus, which comprises hoses, cleaning jets, personnel showers, a collapsible rubberized fabric tank, and capable of dispensing water; or a combination thereof. The ABC-M11, M13 and M280 decontamination equipment packages are generally used for equipment (e.g., vehicles), the M258A1 and M17 decontamination equipment packages are generally used for equipment and/or personnel, and the ABC-M12A1 and M291 decontamination equipment packages are generally used for personnel (Yang, Y. C. et al., 1992).

### Y. SPECIFIC EXAMPLES

The general effectiveness of various embodiments is demonstrated in the following Examples. Some methods for preparing compositions are illustrated. Starting materials are made according to procedures known in the art or as illustrated herein. The following Examples are provided so that the embodiments might be more fully understood. These Examples are illustrative only and should not be construed as limiting in any way, as other material formulations such as a polymeric material, a surface treatment (e.g., a different paint formulation), and/or a filler, comprising different biomolecular compositions (e.g., a different purified or partly purified enzyme, a different cell-based particulate material comprising an enzyme, a peptide, a polypeptide) may be prepared.

### Example 1

This Example demonstrates the use of a coating comprising a lipase, and the enzymatic activity conferred to the coating comprising the lipase by detection of triglyceride breakdown through monitoring pH.

The equipment/reagents were as follows: pH meter; shaker; Lightin Lab Master paint mixer; phenol red (Sigma-Aldrich; Catalog #-P3532), 1.128 mM in distilled water, pH=7.0; lipase (Sigma-Aldrich; Catalog #-L3126), Sherwin Williams acrylic latex paint; sodium hydroxide; hydrochloric acid; isopropyl alcohol; and vegetable oil. The solutions used in measuring pH changes included a phenol red stock solution, 1.128 mM in distilled water, pH=7.0.

The procedure for preparation of the surfaces coated with paint either comprising lipase or not (control paint) was as follows: first, 100 mg/ml, 50 mg/ml, and 0 mg/ml lipase solutions in paint were made; second, solutions were mixed for 3 minutes; third, paints were spread to 8 mils thickness and allowed to dry for 96 hours, and fourth, 1 cm×4 cm coupons were cut from the paint film.

The pre-experimental set-up included the following steps: first, a 1 cm×4 cm piece of film of each lipase concentration was placed in a 15 ml eppendorf tube in triplicate; second, 10 ml ddH₂O was added inside the eppendorf tube; third, tubes on shaker were set for 24 hours, and fourth, after 24 hours, the water from the tube was removed and the film placed in a new 15 ml eppendorf tube. For measuring the control paint (no lipase) samples, the following steps were conducted: first, 5 ml of phenol red stock solution was added into a 15 ml eppendorf tube; second, 5 ml of phenol red stock solution with 100 μl vegetable oil was added into a 15 ml eppendorf tube; third, a 1 cm×4 cm piece of paint film (no lipase) from both the washed and non-washed films was added into a 15 ml eppendorf tube in triplicate; fourth, 5 ml of the phenol red stock solution was added into the 15 ml eppendorf tubes along with 100 μl vegetable oil; and fifth, the tubes were set on a shaker for 24 hours. To measure the paint samples comprising lipase: first, a 1 cm×4 cm piece of the 50 mg/ml paint film, both washed and unwashed, was added into a 15 ml eppendorf tube; second, a 1 cm×4 cm piece of the 100 mg/ml paint film, both washed and unwashed, was added into a 15 ml eppendorf tube; third, 5 ml of the Phenol Red stock solution was added into each tube along with 100 μl vegetable oil; and fourth, the tubes were set on shaker for 24 hours. For both the control paint and lipase paint samples, the pH of each sample was recorded at 24 hours.

Phenol Red comprises a pH indicator that is yellow in color below pH 6.8 and red in color above pH 8.2. Setting the pH at 7.0 right before the 6.8 end point would demonstrate a color change if the solution becomes slightly more acidic. If in fact the triglycerides are being broken down into free fatty acids by lipase, the pH of the solution should go down, thus exhibiting a color change. In the presence of a paint film with no lipase, the pH of the phenol red solution rose from 7 to almost 9. The pH of the tubes with lipase in them were both substantially lower than the control tubes, demonstrating that the triglycerides were broken down into fatty acids, decreasing the pH of the solutions. All lipase impregnated coatings demonstrated catalytic activity. Washing the coating films with water decreased their effectiveness but the films were still active. Further, vegetable oil was spread over panels that were either control (no lipase) or lipase impregnated. After a day, the lipase impregnated panels were dry while the control panels were still visibly full of oil. It is also contemplated that greater loads of lipase, such as, for example, 200 mg/ml, 100 mg/ml, and 50 mg/ml lipase, may be used.

TABLE 9
Samples

| | No washing cycle | 24 hr washing cycle |
|---|---|---|
| Sample | pH at 24 hr | pH at 24 hr |
| Control | 8.87 + 0.01 | 8.78 + 0.04 |
| 50 mg/ml Lipase | 6.80 + 0.05 | 7.25 + 0.21 |
| 100 mg/ml Lipase | 6.70 + 0.05 | 6.63 + 0.07 |

TABLE 10
pH Buffer

| Sample | pH |
|---|---|
| Phenol Red | 7.07 |
| Phenol Red w/oil | 7.08 |

### Example 2

This Example demonstrates the use of a coating comprising a lipase, and the enzymatic activity conferred to the coating comprising the lipase by detection of the hydrolysis of 4-nitrophenyl palmitate through monitoring pH.

The equipment/reagents were as follows: 40 mM CHES Buffer; bring to pH=9.0 with NaOH; 4-nitrophenyl palmitate (Sigma Product # N2752), 14.5 mM solution in isopropyl alcohol; 4-nitrophenyl acetate; lipase from porcine pancreas (Sigma Product # L3126); Sherwin-Williams acrylic latex paint; 2 mL microtubes; paint spreader (1-8 mils); polypropylene blocks; Lightnin Labmaster Mixer; rotator shaker; pipettes and pipetteman; and centrifuge.

The following paint formulations were evaluated: Sherwin-Williams Acrylic Latex Control (no additive), and Sherwin-Williams Acrylic Latex with 100 mg/mL lipase. The paints were mixed in a plastic 50 ml eppendorf tube with a glass stirring rod for three minutes followed by a paint mixer for three minutes. The paints were spread with a mils spreader to 8 mils thickness onto polypropylene surfaces and were allowed to dry a minimum of 72 hours prior to assay. Coupons were generated as free films from the polypropylene surfaces.

The procedure for the preparation of the blank (control) samples was: adding 500 ul 40 mM CHES, 400 ul ddH₂O, and 100 ul 14.5 mM p-nitrophenyl palmitate to a 2 ml microtube. The procedure for preparation of the experimental (comprising lipase) samples was: cutting the following free film sizes for the 100 mg/ml lipase films—1 cm×3 cm, 1 cm×2 cm, and 1 cm×1 cm, and for the control film (no lipase)—1 cm×3 cm; placing the free films into labeled 2 mL microtubes, where each of the coupon sizes were tested in triplicate; adding 500ul 40 mM CHES to each microtube; adding 400 ul ddH2O to each microtube; adding 100 ul 14.5 mM p-nitrophenyl palmitate to each microtube; and setting microtubes on a shaker. At each time point, tubes were placed in a centrifuge for 5 minutes at 13,000 RPM. A 100ul was removed from each tube and the absorbance of the reaction product p-nitrophenol read at 405 nm in a 96-well plate.

The tables below shows the activity of each sample. The measured rates of reaction for the free films without any lipase were essentially baseline, exhibiting no destruction of the 4-nitrophenol palmitate. All lipase impregnated coatings demonstrated catalytic activity. The specific activity per centimeter basis was consistent within the different sample sizes.

TABLE 11A
Lipase Activity in Sherwin-Williams Latex pNP Absorbance at 405 nm

| Time | | | | | | | | | |
|---|---|---|---|---|---|---|---|---|---|
| (min) | 1 cm × 3 cm Lipase | | | 1 cm × 2 cm Lipase | | | 1 cm × 1 cm Lipase | | |
| 1 | 0.2314 | 0.3159 | 0.2781 | 0.3146 | 0.4118 | 0.3865 | 0.4265 | 0.3141 | 0.2917 |
| 30 | 0.2511 | 0.3337 | 0.2615 | 0.2850 | 0.3465 | 0.3523 | 0.3849 | 0.2723 | 0.3136 |
| 60 | 0.2625 | 0.3365 | 0.2794 | 0.2984 | 0.3451 | 0.3494 | 0.3833 | 0.2826 | 0.2873 |
| 120 | 0.2674 | 0.3351 | 0.3180 | 0.2960 | 0.3342 | 0.3361 | 0.3680 | 0.2867 | 0.2657 |
| 210 | 0.2949 | 0.3502 | 0.3057 | 0.2946 | 0.3306 | 0.3304 | 0.3527 | 0.2792 | 0.2329 |
| 1200 | 0.4051 | 0.5281 | 0.4568 | 0.3361 | 0.3308 | 0.3374 | 0.3016 | 0.3066 | 0.2159 |

TABLE 11B
Lipase Activity in Sherwin-Williams Latex
pNP Absorbance at 405 nm

| Time | | | | | | |
|---|---|---|---|---|---|---|
| (min) | 1 cm × 3 cm Control | | | Blank | | |
| 1 | 0.3718 | 0.4458 | 0.2327 | 0.3154 | 0.4142 | 0.3773 |
| 30 | 0.3119 | 0.3631 | 0.2172 | 0.2757 | 0.3442 | 0.3069 |
| 60 | 0.2852 | 0.3380 | 0.2025 | 0.2674 | 0.3307 | 0.2767 |
| 120 | 0.2473 | 0.2572 | 0.1707 | 0.2748 | 0.3259 | 0.2780 |
| 210 | 0.1707 | 0.1996 | 0.1542 | 0.2621 | 0.3007 | 0.2616 |
| 1200 | 0.0541 | 0.0552 | 0.0590 | 0.2374 | 0.2640 | 0.2264 |

TABLE 12
Lipase Average Activity in Sherwin-Williams Latex
pNP Absorbance at 405 nm

| Time | Lipase | Control | |
|---|---|---|---|
| (min) | 1 cm × 3 cm | 1 cm × 3 cm | Blank |
| 1 | 0.2751 | 0.3501 | 0.3690 |
| 30 | 0.2821 | 0.2974 | 0.3089 |
| 60 | 0.2928 | 0.2752 | 0.2916 |
| 120 | 0.3068 | 0.2251 | 0.2929 |
| 210 | 0.3169 | 0.1748 | 0.2748 |
| 1200 | 0.4633 | 0.0561 | 0.2426 |

TABLE 13A
Lipase Activity in Sherwin-Williams Latex pNP Absorbance at 405 nm

| Time | | | | | | | | | |
|---|---|---|---|---|---|---|---|---|---|
| (min) | 1 cm × 3 cm Lipase | | | 1 cm × 2 cm Lipase | | | 1 cm × 1 cm Lipase | | |
| 0 | | | | | | | | | |
| 30 | 0.1685 | 0.2200 | 0.1654 | 0.2135 | 0.1494 | 0.1457 | 0.1271 | 0.0711 | 0.1389 |
| 60 | 0.2287 | 0.1822 | 0.2027 | 0.1570 | 0.2008 | 0.1554 | 0.1500 | 0.1284 | 0.0758 |
| 120 | 0.2044 | 0.2208 | 0.2487 | 0.1694 | 0.1926 | 0.2007 | 0.1126 | 0.0771 | 0.0859 |
| 225 | 0.2521 | 0.2621 | 0.2620 | 0.2707 | 0.1920 | 0.1746 | 0.1779 | 0.1654 | 0.1611 |
| 1200 | 0.3917 | 0.3579 | 0.3735 | 0.2315 | 0.2607 | 0.2682 | 0.1335 | 0.1702 | 0.1300 |

TABLE 13B
Lipase Activity in Sherwin-Williams Latex pNP
Absorbance at 405 nm

| Time | | | | | | |
|---|---|---|---|---|---|---|
| (min) | 1 cm × 3 cm Control | | | Blank | | |
| 0 | | | | 0.1114 | 0.0981 | 0.1269 |
| 30 | 0.1551 | 0.1628 | 0.1173 | 0.1410 | 0.1022 | 0.1204 |
| 60 | 0.1198 | 0.0987 | 0.1029 | 0.0974 | 0.1278 | 0.1119 |
| 120 | 0.1365 | 0.1082 | 0.1192 | 0.1487 | 0.1284 | 0.0995 |
| 225 | 0.0680 | 0.0688 | 0.0602 | 0.1129 | 0.0788 | 0.1231 |
| 1200 | 0.0514 | 0.0521 | 0.0599 | 0.1008 | 0.1106 | 0.0626 |

TABLE 14
Lipase Activity in Sherwin-Williams Latex pNP Average Absorbance at 405 nm
and Standard Deviations

| | Average | | | | | SD | | | | |
|---|---|---|---|---|---|---|---|---|---|---|
| | Lipase | | | Control | | Lipase | | | Control | |
| Time | 1 cm × | 1 cm × | 1 cm × | 1 cm × | | 1 cm × | 1 cm × | 1 cm × | 1 cm × | |
| (min) | 3 cm | 2 cm | 1 cm | 3 cm | Blank | 3 cm | 2 cm | 1 cm | 3 cm | Blank |
| 0 | 0.1121 | 0.1121 | 0.1121 | 0.1121 | 0.1121 | 0.0144 | 0.0144 | 0.0144 | 0.0144 | 0.0144 |
| 30 | 0.1846 | 0.1695 | 0.1124 | 0.1451 | 0.1212 | 0.0307 | 0.0381 | 0.0362 | 0.0244 | 0.0194 |
| 60 | 0.2045 | 0.1711 | 0.1181 | 0.1071 | 0.1124 | 0.0233 | 0.0258 | 0.0382 | 0.0112 | 0.0152 |
| 120 | 0.2246 | 0.1876 | 0.0919 | 0.1213 | 0.1255 | 0.0224 | 0.0162 | 0.0185 | 0.0143 | 0.0247 |
| 225 | 0.2587 | 0.2124 | 0.1681 | 0.0657 | 0.1049 | 0.0057 | 0.0512 | 0.0087 | 0.0048 | 0.0232 |
| 1200 | 0.3744 | 0.2535 | 0.1446 | 0.0545 | 0.0913 | 0.0169 | 0.0194 | 0.0223 | 0.0047 | 0.0254 |

TABLE 15A
Lipase Activity in Sherwin-Williams Latex pNP Absorbance at 405 nm and Initial
Slopes

| | Lipase | | | | | | | | |
|---|---|---|---|---|---|---|---|---|---|
| Time (min) | 1 cm × 3 cm | | | 1 cm × 2 cm | | | 1 cm × 1 cm | | |
| 0 | 0.1121 | 0.1121 | 0.1121 | 0.1121 | 0.1121 | 0.1121 | 0.1121 | 0.1121 | 0.1121 |
| 225 | 0.2521 | 0.2621 | 0.2620 | 0.2707 | 0.1920 | 0.1746 | 0.1779 | 0.1654 | 0.1611 |
| Slope | 0.0006 | 0.0007 | 0.0007 | 0.0007 | 0.0004 | 0.0003 | 0.0003 | 0.0002 | 0.0002 |
| (ΔAbs/Δmin) | | | | | | | | | |
| U (umol/ | 0.1362 | 0.1459 | 0.1458 | 0.1543 | 0.0777 | 0.0608 | 0.0640 | 0.0519 | 0.0477 |
| min) | | | | | | | | | |
| U/cm² | 0.0454 | 0.0486 | 0.0486 | 0.0772 | 0.0389 | 0.0304 | 0.0640 | 0.0519 | 0.0477 |

TABLE 15B
Lipase Activity in Sherwin-Williams Latex pNP Absorbance at 405 nm
and Initial Slopes

| Time (min) | 1 cm × 3 cm Control | | | Blank | | |
|---|---|---|---|---|---|---|
| 0 | 0.1121 | 0.1121 | 0.1121 | 0.1121 | 0.1121 | 0.1121 |
| 225 | 0.0680 | 0.0688 | 0.0602 | 0.1129 | 0.0788 | 0.1231 |
| Slope | −0.0002 | −0.0002 | −0.0002 | 0.0000 | −0.0001 | 0.0000 |
| (ΔAbs/Δmin) | | | | | | |
| U (umol/ | −0.0429 | −0.0421 | −0.0505 | 0.0008 | −0.0324 | 0.0107 |
| min) | | | | | | |
| U/cm² | | | | | | |

TABLE 16
Sample Activity

| Sample | U (μmol/min) | U (μmol/min)/cm² |
|---|---|---|
| 1 cm × 3 cm; with lipase | 0.1427 ± 0.0056 | 0.0476 ± 0.0019 |
| 1 cm × 2 cm; with lipase | 0.0976 ± 0.0498 | 0.0488 ± 0.0249 |
| 1 cm × 1 cm; with lipase | 0.0545 ± 0.0085 | 0.0545 ± 0.0085 |
| 1 cm × 3 cm; no lipase | −0.0452 ± 0.0046 | |
| Blank | −0.0070 ± 0.0226 | |

The reaction containing the 1 cm×3 cm free-film with lipase went to 50% completion. This is due to the nature of the insolubility of 4-nitrophenyl palmitate. Particles of 4-nitrophenyl palmitate were present in all microtubes due to precipitation when it comes in contacts with water. The 1 cm×1 cm free-film was likely too small a film size, although the microtube was visually yellow, the data did not support the fact that the reaction did in fact take place. 4-nitrophenyl palmitate was originally used, but it self-hydrolyzed in water. Further, vegetable oil was spread over panels that were either control (no lipase) or lipase impregnated. After a day, the lipase impregnated panels were dry while the control panels were still visibly full of oil. It is also contemplated that greater loads of lipase, such as, for example, 200 mg/ml, 100 mg/ml, and 50 mg/ml lipase, may be used.

### Example 3

This Example is directed to additional examples of lipolytic enzyme encoding nucleic acid sequences (e.g., full length cDNAs for lipolytic genes) that are contemplated for use in the expression of recombinant lipolytic enzymes, as well as source organisms for endogenously produced lipolytic enzymes, for use in the preparation of biomolecular compositions.

TABLE 17
Lipolytic Enzyme Genes and Source Organisms

| Lipolytic Enzyme | Characteristics | Source | Accession No |
|---|---|---|---|
| Carboxylesterase | CXE4 gene | Actinidia deliciosa | DQ279917 |
| Carboxylesterase | CXE3 gene | Actinidia deliciosa | DQ279916 |
| Carboxylesterase | | Aedes aegypti | XM_001647935 |
| Carboxylesterase | carboxylesterase-6 | Aedes aegypti | XM_001656069 |
| Carboxylesterase | malathion-resistant | Anisopteromalus | AF064524 |
| | | calandrae | |
| Carboxylesterase | malathion-susceptible | Anisopteromalus | AF064523 |
| | | calandrae | |
| Carboxylesterase | CarE-S gene | Aphis gossypii | AY049740 |
| Carboxylesterase | organophosphorus insecticide | Aphis gossypii | AB245435 |
| | super-susceptible strain | | |
| Carboxylesterase | organophosphorus insecticide | Aphis gossypii | AB245434 |
| | susceptible strain | | |
| Carboxylesterase | | Arabidopsis thaliana | NM_001036026 |
| Carboxylesterase | est-1 gene, GeneID: 1484085 | Archaeoglobus fulgidus | NC_000917 |
| Carboxylesterase | estA gene, GeneD: 1484939 | Archaeoglobus fulgidus | NC_000917 |
| Carboxylesterase | est-3 gene, GeneID: 1485568 | Archaeoglobus fulgidus | NC_000917 |
| Carboxylesterase | est-2 gene, GeneID: 1484765 | Archaeoglobus fulgidus | NC_000917 |
| Carboxylesterase | | Aspergillus clavatus | XM_001271426 |
| | | NRRL 1 | |
| Carboxylesterase | COE gene | Athalia rosae | AB208651 |
| Carboxylesterase | | Bombyx mandarina | EF157830 |
| Carboxylesterase | | Bombyx mori | DQ443360 |
| Carboxylesterase | carboxylesterase 2, intestine, | Bos taurus | BC102288 |
| | liver | | |
| Carboxylesterase | | Caenorhabditis elegans | NM_071999 |
| | | B0238.1 | |
| Carboxylesterase | | Caenorhabditis elegans | NM_068642 |
| | | C17H12.4 | |
| Carboxylesterase | | Caenorhabditis elegans | NM_171976 |
| | | F55F3.2a | |
| Carboxylesterase | | Caenorhabditis elegans | NM_068669 |
| | | T22D1.11 | |
| Carboxylesterase | CESdD1 gene, | Canis familiaris | AB023629 |
| | carboxylesterase D1 | | |
| Carboxylesterase | | Cavia porcellus | AB010634 |
| Carboxylesterase | CES1 gene | Felis catus | AB094147 |
| Carboxylesterase | CES-K1 gene | Felis catus | AB114676 |
| Carboxylesterase | GeneID: 5452002 | Fervidobacterium | NC_009718 |
| | | nodosum Rt17-B1 | |
| Carboxylesterase | | Helicoverpa armigera | EF547544 |
| Carboxylesterase | carboxylesterase 3, brain | Homo sapiens | BC053670 |
| Carboxylesterase | CES5, carboxylesterase 5 | Homo sapiens | AY907669 |
| Carboxylesterase | carboxylesterase 2, intestine, | Homo sapiens | BC032095 |
| | liver | | |
| Carboxylesterase | | Homo sapiens | D50579 |
| Carboxylesterase | carboxylesterase 7 | Homo sapiens | BC117126 |
| Carboxylesterase | | Macaca fascicularis | AB010633 |
| Carboxylesterase | CXE10 gene | Malus pumila | DQ279911 |
| Carboxylesterase | CXE1 gene | Malus pumila | DQ279902 |
| Carboxylesterase | | Mesocricetus auratus | D50577 |
| Carboxylesterase | | Mus musculus | AB023631 |
| Carboxylesterase | carboxylesterase 2 | Mus musculus | BC034182 |
| Carboxylesterase | carboxylesterase ML3 | Mus musculus | AB110073 |
| Carboxylesterase | | Mus musculus | M57960 |
| Carboxylesterase | carboxylesterase 6 | Mus musculus | BC024491 |
| Carboxylesterase | carboxylesterase 5 | Mus musculus | BC055062 |
| Carboxylesterase | carboxylesterase 3 | Mus musculus | BC019198 |
| Carboxylesterase | carboxylesterase 1 | Mus musculus | BC026897 |
| Carboxylesterase | MdaE7 gene | Musca domestica | AF133341 |
| Carboxylesterase | | Neosartorya fischeri | XM_001260356 |
| | | NRRL 181 | |
| Carboxylesterase | Liver | Oryctolagus cuniculus | AF036930 |
| Carboxylesterase | CXE gene | Paeonia suffruticosa | EU072921 |
| | | clone 199 | |
| Carboxylesterase | Est gene | Pseudomonas | AF228666 |
| | | fluorescens | |
| Carboxylesterase | liver microsomal | Rattus norvegicus | U10698 |
| Carboxylesterase | kidney microsomal | Rattus norvegicus | U10697 |
| Carboxylesterase | CESrRL1 gene | Rattus norvegicus | AB023630 |
| Carboxylesterase | ES-4 gene | Rattus norvegicus | BC128711 |
| Carboxylesterase | | Rattus norvegicus | AF479659 |
| Carboxylesterase | carboxylesterase 3 | Rattus norvegicus | BC061789 |
| Carboxylesterase | rCES2 gene | Rattus norvegicus | AB191005 |
| Carboxylesterase | | Spodoptera exigua | EF580101 |
| Carboxylesterase | | Spodoptera litura | DQ445461 |
| Carboxylesterase | SshEstI gene | Sulfolobus shibatae | AB166870 |
| Carboxylesterase | GeneID: 1453975 | Sulfolobus solfataricus | NC_002754 |
| | | P2 | |
| Carboxylesterase | | Sus scrofa | AF064741 |
| Carboxylesterase | GeneID: 2774935 | Thermus thermophilus | NC_005835 |
| | | HB27 | |
| Carboxylesterase | GeneID: 2775775 | Thermus thermophilus | NC_005835 |
| | | HB27 | |
| Carboxylesterase | GeneID: 3168028 | Thermus thermophilus | NC_006461 |
| | | HB8 | |
| Carboxylesterase | CXE1 gene | Vaccinium corymbosum | DQ279901 |
| Carboxylesterase | secreted salivary | Xenopsylla cheopis | EF179418 |
| | | clone XC-184 | |
| carboxylesterase/ | GeneID: 3474139 | Sulfolobus | NC_007181 |
| lipase | | acidocaldarius | |
| Lipase | | Aedes aegypti | XM_001651298 |
| Lipase | | Aedes aegypti | XM_001654736 |
| Lipase | Lip gene | Anguilla japonica | AB070722 |
| Lipase | | Antrodia cinnamomea | EF088667 |
| Lipase | | Arabidopsis thaliana | NM_202246 |
| Lipase | lipase 1, LI-tolerant, | Arabidopsis thaliana | NM_111300 |
| | carboxylesterase | | |
| Lipase | extracellular lipase 4; | Arabidopsis thaliana | NM_106241 |
| | acyltransferase/ | | |
| | carboxylesterase/lipase | | |
| Lipase | ATLIP1 gene, lipase 1, | Arabidopsis thaliana | NM_127084 |
| | galactolipase/phospholipase/ | | |
| | lipase | | |
| Lipase | ARAB-1 gene, | Arabidopsis thaliana | NM_102634 |
| | carboxylesterase | | |
| Lipase | | Arabidopsis thaliana | NM_118185 |
| Lipase | DAD1 gene | Arabidopsis thaliana | NM_130045 |
| Lipase | lipase1; carboxylesterase | Arabidopsis thaliana | NM_123464 |
| Lipase | lipB gene | Aspergillus niger | DQ680031 |
| Lipase | lipA gene | Aspergillus niger | DQ680030 |
| Lipase | | Aspergillus tamarii | EU131679 |
| | | isolate FS132 | |
| Lipase | Extracellular | Aureobasidium pullulans | EU082005 |
| | | HN2.3 | |
| Lipase | | Avena sativa | AY566266 |
| Lipase | | Bombyx mandarina | AY945212 |
| Lipase | | Bombyx mori | AY945209 |
| Lipase | bile salt-stimulated lipase | Bos Taurus | BT021633 |
| Lipase | lipase 1 gene | Brassica napus | AY866419 |
| Lipase | lipase 2 | Brassica napus | AY870270 |
| Lipase | SIL1 gene | Brassica rapa subsp. | AY101366 |
| | | Pekinensis | |
| Lipase | | Caenorhabditis elegans | NM_069722 |
| | | B0035.13 | |
| Lipase | | Chenopodium rubrum | AY299194 |
| Lipase | GeneID: 5292515 | Clostridium beijerinckii | NC_009617 |
| | | NCIMB 8052 | |
| Lipase | GeneID: 5396655 | Clostridium botulinum A | NC_009697 |
| | | str. | |
| Lipase | GeneID: 5395737 | Clostridium botulinum A | NC_009697 |
| | | str. | |
| Lipase | GeneID: 5405010 | Clostridium botulinum F | NC_009699 |
| | | str. Langeland | |
| Lipase | GeneID: 4540684 | Clostridium novyi NT | NC_008593 |
| Lipase | Hepatic | Danio rerio | BC053243 |
| Lipase | Gastric | Danio rerio | BC052131 |
| Lipase | Adipose | Gallus gallus | EU240627 |
| Lipase | FGL4 gene | Gibberella zeae | EU191903 |
| Lipase | FGL2 gene | Gibberella zeae | EU191902 |
| Lipase | | Gossypium hirsutum | EU273289 |
| Lipase | Endothelial | Homo sapiens | AF118767 |
| Lipase | | Homo sapiens | AF225418 |
| Lipase | Endothelial | Homo sapiens | BC060825 |
| Lipase | LIPH gene, lipase H | Homo sapiens | EF186229 |
| Lipase | LIPK gene, lipase K | Homo sapiens | EF426482 |
| Lipase | LIPM gene, lipase M, | Homo sapiens | EF426484 |
| Lipase | Pancreatic | Homo sapiens | BC014309 |
| Lipase | hormone-sensitive | Homo sapiens | BC070041 |
| Lipase | bile salt-stimulated lipase | Homo sapiens | BC042510 |
| Lipase | adipose, ATGL gene | Homo sapiens | AY894804 |
| Lipase | Hepatic | Homo sapiens | D83548 |
| Lipase | Lip gene | Kurtzmanomyces sp. I- | AB073866 |
| | | 11 | |
| Lipase | | Leishmania infantum | XM_001467534 |
| | | JPCM5 | |
| Lipase | GeneID: 1474518 | Methanosarcina | NC_003552 |
| | | acetivorans | |
| Lipase | Pancreatic | Mus musculus | BC061061 |
| Lipase | member H | Mus musculus | BC037489 |
| Lipase | hormone sensitive | Mus musculus | BC021642 |
| Lipase | Pancreatic | Mus musculus | AY387690 |
| Lipase | Gastric | Mus musculus | BC061067 |
| Lipase | | Mus musculus | U37386 |
| Lipase | Endothelial | Mus musculus | BC020991 |
| Lipase | Liph gene, lipase H | Mus musculus | AY093499 |
| Lipase | hormone-sensitive | Mus musculus | U08188 |
| Lipase | Lipc gene, hepatic | Mus musculus | AY228765 |
| Lipase | Endothelial | Mus musculus | AF118768 |
| Lipase | cytotoxic T lymphocyte | Mus musculus | M30687 |
| Lipase | | Mus musculus | AY894805 |
| Lipase | Hepatic | Mus musculus | BC094050 |
| Lipase | Lipc gene, hepatic | Mus spretus | AY225159 |
| Lipase | Secretory | Neosartorya fischeri | XM_001257303 |
| | | NRRL 181 | |
| Lipase | Lacrimal | Oryctolagus cuniculus | AF351188 |
| Lipase | Hepatic | Oryctolagus cuniculus | AF041202 |
| Lipase | | Oryctolagus cuniculus | M99365 |
| | | clone TGL-5K | |
| Lipase | Alkaline | Penicillium cyclopium | AF274320 |
| Lipase | Hepatic | Rattus norvegicus | BC088160 |
| Lipase | Lipg gene, endothelial | Rattus norvegicus | AY916123 |
| Lipase | lipRs gene | Rhizopus stolonifer | DQ139862 |
| Lipase | OBL2 gene | Ricinus communis | AY724687 |
| Lipase | OBL1 gene | Ricinus communis | AY360220 |
| Lipase | | Ricinus communis acidic | EF071862 |
| Lipase | | Samia cynthia ricini | DQ149986 |
| | | strain Banma | |
| Lipase | | Schizosaccharomyces | NM_001023305 |
| | | pombe | |
| Lipase | PL-h gene, heart pancreatic | Spermophilus | AF027293 |
| | | tridecemlineatus | |
| Lipase | Pancreatic | Spermophilus | AF395870 |
| | | tridecemlineatus | |
| Lipase | PTL gene, pancreatic | Spermophilus | AF177403 |
| | | tridecemlineatus clone | |
| | | 22A4 | |
| Lipase | PTL gene, pancreatic | Spermophilus | AF177402 |
| | | tridecemlineatus clone | |
| | | 7G5 | |
| Lipase | lipP-1 gene, GeneID: 1453956 | Sulfolobus solfataricus | NC_002754 |
| | | P2 | |
| Lipase | lipP-2 gene, GeneID: 1453979 | Sulfolobus solfataricus | NC_002754 |
| | | P2 | |
| Lipase | ATGL gene, adipose | Sus scrofa | EF583921 |
| Lipase | Lip gene | Thermomyces | AF054513 |
| | | lanuginosus | |
| Lipase | LIP gene | Trichomonas vaginalis | AY870437 |
| Lipase | bile salt-stimulated lipase | Xenopus laevis | BC106664 |
| Lipase | | Xenopus laevis | BC054271 |
| Colipase | Pancreatic | Homo sapiens | BT006812 |
| Colipase | | Homo sapiens | J02883 |
| Colipase | Pancreatic | Mus musculus | BC042935 |
| Colipase | Clps gene | Mus musculus | AF414676 |
| | | C57BL/6J | |
| Colipase | Clps gene | Mus musculus CAST/Ei | AF414677 |
| Colipase | Pancreatic | Oryctolagus cuniculus | L06329 |
| Colipase | Pancreatic | Spermophilus | AF395869 |
| | | tridecemlineatus | |
| Colipase | Pancreatic | Sus scrofa | AF148567 |
| lipase/acylhydrolase | GeneID: 5186955 | Clostridium botulinum A | NC_009495 |
| | | str. | |
| lipoprotein lipase | | Capra hircus | DQ370053 |
| lipoprotein lipase | | Danio rerio | BC064296 |
| lipoprotein lipase | | Felis catus | U42725 |
| lipoprotein lipase | | Homo sapiens | BT006726 |
| lipoprotein lipase | | Mesocricetus auratus | AB194713 |
| lipoprotein lipase | | Mus musculus | BC003305 |
| lipoprotein lipase | | Oncorhynchus mykiss | AF358669 |
| lipoprotein lipase | | Pagrus major | AB054062 |
| lipoprotein lipase | | Papio Anubis | U18091 |
| lipoprotein lipase | | Rattus norvegicus | L03294 |
| lipoprotein lipase | | Sparus aurata | AY495672 |
| lipoprotein lipase | | Sus scrofa breed Duroc | AY559454 |
| lipoprotein lipase | | Sus scrofa breed Large | AY686761 |
| | | White | |
| lipoprotein lipase | | Sus scrofa breed Mei | AY686760 |
| | | Shan | |
| lipoprotein lipase | | Sus scrofa breed | AY559453 |
| | | Tongcheng | |
| lipoprotein lipase | | Thunnus orientalis | AB370192 |
| acylglycerol lipase | | Danio rerio | BC049487 |
| acylglycerol lipase | | Danio rerio clone | AY398382 |
| | | RK135A2B08 | |
| acylglycerol lipase | | Homo sapiens | BC006230 |
| acylglycerol lipase | | Leishmania infantum | XM_001467371 |
| | | JPCM5 | |
| acylglycerol lipase | | Mus musculus | BC057965 |
| acylglycerol lipase | | Rattus norvegicus | BC107920 |
| acylglycerol lipase | Mgl2 gene | Rattus norvegicus | AY081195 |
| hormone sensitive | LIPE gene | Bos taurus | EF140760 |
| lipase | | | |
| hormone sensitive | testicular isoform | Rattus norvegicus | U40001 |
| lipase | | | |
| hormone sensitive | | Rattus norvegicus | BC078888 |
| lipase | | | |
| hormone sensitive | HSL gene | Spermophilus | AF177401 |
| lipase | | tridecemlineatus | |
| hormone sensitive | | Sus scrofa breed Large | AY686758 |
| lipase | | White | |
| hormone sensitive | | Sus scrofa breed Mei | AY686759 |
| lipase | | Shan | |
| hormone sensitive | | Tetrahymena | XM_001031360 |
| lipase | | thermophila SB210 | |
| phospholipase A₁ | | Arabidopsis thaliana | AF421148 |
| phospholipase A₁ | PLA1 gene | Aspergillus oryzae | E16314 |
| phospholipase A₁ | member A | Bos Taurus | BT020950 |
| phospholipase A₁ | phosphatidic acid-preferring | Bos Taurus | AF045022 |
| phospholipase A₁ | | Brassica rapa | EF492990 |
| phospholipase A₁ | intracellular, ipla-1 | Caenorhabditis elegans | EU180219 |
| phospholipase A₁ | PLA1 gene | Capsicum annuum | EF595843 |
| phospholipase A₁ | | Danio rerio | BC066406 |
| phospholipase A₁ | phosphatidylserine-specific, | Homo sapiens | AF035268 |
| phospholipase A₁ | member A | Homo sapiens | BC047703 |
| phospholipase A₁ | | Homo sapiens | E16580 |
| phospholipase A₁ | membrane-bound, | Homo sapiens | AY036912 |
| | phosphatidic acid selective | | |
| phospholipase A₁ | beta, membrane-associated | Homo sapiens | AY197607 |
| phospholipase A₁ | phosphatidylserine-specific, | Homo sapiens | AF035269 |
| | deltaC, PS-PLA1deltaC gene | | |
| phospholipase A₁ | Ps-pla1 gene, | Mus musculus | AF063498 |
| | phosphatidylserine-specific | | |
| phospholipase A₁ | | Mus musculus | BC030670 |
| phospholipase A₁ | | Nicotiana tabacum | AF468223 |
| phospholipase A₁ | | Polistes annularis | AF174527 |
| phospholipase A₁ | venom gland | Polybia paulista | EF101736 |
| phospholipase A₁ | phosphatidylserine-specific | Rattus norvegicus | BC078727 |
| phospholipase A₁ | Extracellular | Serratia liquefaciens | M23640 |
| phospholipase A₁ | | Vespula vulgaris | L43561 |
| phospholipase A₂ | | Acanthaster planci | AB211367 |
| phospholipase A₂ | | Adamsia carciniopado | AF347072 |
| phospholipase A₂ | ipla2 gene, 85 kda calcium- | Aedes aegypti | XM_001656230 |
| | independent | | |
| phospholipase A₂ | Isozyme | Aipysurus eydouxii | AY561163 |
| | | clone c10 | |
| phospholipase A₂ | | Apis mellifera | AF438408 |
| phospholipase A₂ | phospholipase A2 alpha | Arabidopsis thaliana | AY344842 |
| phospholipase A₂ | ASPLA1 gene | Austrelaps superbus | AF184127 |
| phospholipase A₂ | | Bitis gabonica | AY429476 |
| phospholipase A₂ | group IVA, PLA2G4A gene | Bos taurus | AY363688 |
| phospholipase A₂ | lysosomal, LPLA2 gene | Bos taurus | AY072914 |
| phospholipase A₂ | Acidic | Bothriechis schlegelii | AY764137 |
| phospholipase A₂ | N6 basic | Bothriechis schlegelii | AY355168 |
| phospholipase A₂ | Hypotensive | Bothrops jararacussu | AY145836 |
| phospholipase A₂ | Myotoxic | Bothrops jararacussu | AY185201 |
| phospholipase A₂ | Cytosolic | BrachyDanio rerio | U10330 |
| phospholipase A₂ | | Bungarus caeruleus | AF297663 |
| phospholipase A₂ | Phospholipase A2 II | Bungarus fasciatus | AF387594 |
| phospholipase A₂ | Antimicrobial | Bungarus fasciatus | DQ868667 |
| phospholipase A₂ | phospholipase A2 I | Bungarus fasciatus | AF387595 |
| phospholipase A₂ | Lysosomal | Canis familiaris | AY217754 |
| phospholipase A₂ | | Cavia sp. | D00740 |
| phospholipase A₂ | | Cerrophidion godmani | AY764139 |
| | | D1E6b | |
| phospholipase A₂ | PLA2 gene | Chlamydomonas | XM_001699805 |
| | | reinhardtii | |
| phospholipase A₂ | ppla2-1 gene | Chrysophrys major | AB050632 |
| phospholipase A₂ | gillpla2 gene | Chrysophrys major | AB050633 |
| phospholipase A₂ | | Chrysophrys major | AB009286 |
| phospholipase A₂ | | Crotalus viridis viridis | AF403137 |
| | | isolate E6h | |
| phospholipase A₂ | | Crotalus viridis viridis | AF403138 |
| | | isolate N6 | |
| phospholipase A₂ | Acidic | Crotalus viridis viridis | AY120875 |
| | | strain E6e | |
| phospholipase A₂ | phospholipase A2-I | Daboia russellii | DQ365974 |
| phospholipase A₂ | Acidic | Daboia russellii from | DQ090659 |
| | | India | |
| phospholipase A₂ | Basic | Daboia russellii from | DQ090660 |
| | | India | |
| phospholipase A₂ | Acidic | Daboia russellii | DQ090654 |
| | | siamensis from | |
| | | Myanmar | |
| phospholipase A₂ | | Daboia russellii | DQ090657 |
| | | siamensis from | |
| | | Myanmar | |
| phospholipase A₂ | group VI, cytosolic, calcium- | Danio rerio | BC067375 |
| | independent | | |
| phospholipase A₂ | group XIIB | Danio rerio | BC093127 |
| phospholipase A₂ | | Echis carinatus | AY268946 |
| phospholipase A₂ | acidic, PLA2-4 gene | Echis carinatus | AF539919 |
| | | sochureki | |
| phospholipase A₂ | acidic, PLA2-5 gene | Echis ocellatus | AF539921 |
| phospholipase A₂ | acidic, PLA2-5 gene | Echis pyramidum | AF539920 |
| | | leakeyi | |
| phospholipase A₂ | plaA gene | Emericella nidulans | AB101663 |
| phospholipase A₂ | Secretory | Equus caballus | EF428565 |
| phospholipase A₂ | PLA2 gene | Equus caballus cytosolic | AF092539 |
| phospholipase A₂ | Cytosolic | Gallus gallus | U10329 |
| phospholipase A₂ | group VI, cytosolic, calcium- | Homo sapiens | BC051904 |
| | independent | | |
| phospholipase A₂ | Ca²⁺-independent, long | Homo sapiens | AF102989 |
| | isoform, iPLA2 gene | | |
| phospholipase A₂ | calcium-independent | Homo sapiens | AF064594 |
| phospholipase A₂ | calcium-independent | Homo sapiens | AB041261 |
| phospholipase A₂ | cPLA2 delta gene; cytosolic | Homo sapiens | AB090876 |
| phospholipase A₂ | beta, cytosolic | Homo sapiens | AF121908 |
| phospholipase A₂ | group XIIB | Homo sapiens | BC093996 |
| phospholipase A₂ | Ca²⁺-dependent | Homo sapiens | U03090 |
| phospholipase A₂ | group IVB, cytosolic | Homo sapiens | BC025290 |
| phospholipase A₂ | liver platelet | Homo sapiens | AY656695 |
| phospholipase A₂ | PLA2 gene, group IID | Homo sapiens | AF112982 |
| | secretory | | |
| phospholipase A₂ | | Homo sapiens | AF188625 |
| phospholipase A₂ | group IB, pancreas | Homo sapiens | BC005386 |
| phospholipase A₂ | group IIA, platelets, synovial | Homo sapiens | BC005919 |
| | fluid | | |
| phospholipase A₂ | group IID | Homo sapiens | BC025706 |
| phospholipase A₂ | group XIIA | Homo sapiens | BC017218 |
| phospholipase A₂ | group IVA, cytosolic, calcium- | Homo sapiens | BC114340 |
| | dependent | | |
| phospholipase A₂ | group X | Homo sapiens | BC106731 |
| phospholipase A₂ | group IVC, cytosolic, calcium- | Homo sapiens | BC063416 |
| | independent | | |
| phospholipase A₂ | gamma, cytosolic | Homo sapiens | AF058921 |
| phospholipase A₂ | group IVD, cytosolic | Homo sapiens | BC034571 |
| phospholipase A₂ | group IVE | Homo sapiens | BC101612 |
| phospholipase A₂ | group IVF | Homo sapiens | BC146648 |
| phospholipase A₂ | group V | Homo sapiens | BC036792 |
| phospholipase A₂ | gamma, membrane-associated | Homo sapiens | AF263613 |
| | calcium-independent | | |
| phospholipase A₂ | group III | Homo sapiens | BC025316 |
| phospholipase A₂ | | Lapemis hardwickii | EF405872 |
| phospholipase A₂ | pla2 gene | Laticauda semifasciata | AB037409 |
| phospholipase A₂ | | Micrurus corallines | AY157830 |
| phospholipase A₂ | group IB, pancreas | Mus musculus | BC145908 |
| phospholipase A₂ | Pla2g10 gene; group X | Mus musculus | AF166097 |
| | secreted | | |
| phospholipase A₂ | group V | Mus musculus | BC030899 |
| phospholipase A₂ | group IID | Mus musculus | BC111806 |
| phospholipase A₂ | group IIA, platelets, synovial | Mus musculus | BC045156 |
| | fluid | | |
| phospholipase A₂ | Fksg71 gene, group XIII | Mus musculus | AF339738 |
| | secreted | | |
| phospholipase A₂ | group VI | Mus musculus | BC052845 |
| phospholipase A₂ | group V | Mus musculus | AF162713 |
| phospholipase A₂ | group IVA, cytosolic, calcium- | Mus musculus | BC003816 |
| | dependent | | |
| phospholipase A₂ | group XIIB gene | Mus musculus | BC021592 |
| phospholipase A₂ | Pla2g5 gene, group 5 | Mus musculus | U66873 |
| phospholipase A₂ | Pla2g4e gene, cytosolic | Mus musculus | AB195277 |
| phospholipase A₂ | group XIIA | Mus musculus | BC026812 |
| phospholipase A₂ | Pla2 gene, secretory | Mus musculus | AF112984 |
| phospholipase A₂ | Pla2g4f gene cytosolic | Mus musculus | AB195278 |
| phospholipase A₂ | Lpla2, lysosomal | Mus musculus | AF468958 |
| phospholipase A₂ | sPLA2 gene, mutant secretory | Mus musculus | U32359 |
| | group II | | |
| phospholipase A₂ | non-pancreatic secreted type II | Mus musculus | U28244 |
| phospholipase A₂ | Pla2 gene, pancreatic | Mus musculus | AF187852 |
| phospholipase A₂ | group X | Mus musculus | BC028879 |
| phospholipase A₂ | group IVD | Mus musculus | BC113160 |
| phospholipase A₂ | group IIC | Mus musculus | BC029347 |
| phospholipase A₂ | Pla2 gene, group IID secretory | Mus musculus | AF112983 |
| phospholipase A₂ | group I | Mus musculus | AF162712 |
| phospholipase A₂ | group IVC, cytosolic, calcium- | Mus musculus | BC117808 |
| | independent | | |
| phospholipase A₂ | | Mus musculus | D78647 |
| phospholipase A₂ | Pla2g2f gene, group IIF | Mus musculus | AF166099 |
| | secreted | | |
| phospholipase A₂ | group IVB, cytosolic | Mus musculus | BC016255 |
| phospholipase A₂ | cytosolic, phospholipase A₂ | Mus musculus | DQ888308 |
| | beta | | |
| phospholipase A₂ | Pla2g2e gene, group IIE | Mus musculus | AF166098 |
| | secreted | | |
| phospholipase A₂ | Pla2g4d gene, cytosolic | Mus musculus | AB195276 |
| phospholipase A₂ | 85 kDa calcium-independent | Mus musculus | U88624 |
| phospholipase A₂ | testis-specific low molecular | Mus musculus | U18119 |
| | weight | | |
| phospholipase A₂ | group IIF | Mus musculus | BC125567 |
| phospholipase A₂ | group IIE | Mus musculus | BC027524 |
| phospholipase A₂ | group III | Mus musculus | BC079556 |
| phospholipase A₂ | group XII-1 | Mus musculus strain | AY007381 |
| | | AKR | |
| phospholipase A₂ | | Mytilus edulis | DQ172904 |
| phospholipase A₂ | NnkPLA-II gene | Naja kaouthia | AB011389 |
| phospholipase A₂ | pla2 gene, clone 1 | Naja naja | L42006 |
| phospholipase A₂ | t1pla2 gene | Nicotiana tabacum | AB190177 |
| phospholipase A₂ | APLA2-1 gene, acidic | Ophiophagus Hannah | AF302908 |
| phospholipase A₂ | PLA2 gene | Ophiophagus Hannah | AF297034 |
| phospholipase A₂ | | Ornithodoros parkeri | EF633936 |
| | | clone OP-525 | |
| phospholipase A₂ | PLA2 gene, microsomal-bound | Oryctolagus cuniculus | AY739721 |
| | CA²⁺-independent | | |
| phospholipase A₂ | group VIB calcium- | Oryctolagus cuniculus | AY738591 |
| | independent | | |
| phospholipase A₂ | group VIA2 | Oryctolagus cuniculus | AY744674 |
| phospholipase A₂ | inpla2 gene | Pagrus major | AB236358 |
| phospholipase A₂ | | Patiria pectinifera | AB022278 |
| phospholipase A₂ | PLA2 gene | Polyandrocarpa | AB107990 |
| | | misakiensis | |
| phospholipase A₂ | | Protobothrops | DQ299948 |
| | | mucrosquamatus | |
| phospholipase A₂ | group V | Rattus norvegicus | BC085745 |
| phospholipase A₂ | group 2C | Rattus norvegicus | BC097325 |
| phospholipase A₂ | aiPLA2 gene, acidic calcium- | Rattus norvegicus | AF014009 |
| | independent | | |
| phospholipase A₂ | group IID | Rattus norvegicus | BC091221 |
| phospholipase A₂ | Pancreatic | Rattus norvegicus | D00036 |
| phospholipase A₂ | group IVA, cytosolic, calcium- | Rattus norvegicus | BC070940 |
| | dependent | | |
| phospholipase A₂ | group IVA, cytosolic, calcium- | Rattus norvegicus | BC070940 |
| | dependent | | |
| phospholipase A₂ | 14 kDa | Rattus norvegicus | U07798 |
| phospholipase A₂ | calcium-independent | Rattus norvegicus | U97146 |
| phospholipase A₂ | group VI | Rattus norvegicus | BC081916 |
| phospholipase A₂ | group X secreted | Rattus norvegicus | AF166100 |
| phospholipase A₂ | Lysosomal | Rattus norvegicus | AY490816 |
| phospholipase A₂ | Cytosolic | Rattus norvegicus | U38376 |
| phospholipase A₂ | | Sistrurus catenatus | AY508692 |
| | | tergeminus | |
| phospholipase A₂ | N6a gene, basic | Sistrurus catenatus | AY355170 |
| | | tergeminus | |
| phospholipase A₂ | G6D49 gene | Trimeresurus | AY355179 |
| | | borneensis | |
| phospholipase A₂ | Acidic | Trimeresurus | AY355178 |
| | | borneensis E6 | |
| phospholipase A₂ | | Trimeresurus flavoviridis | D10070 |
| phospholipase A₂ | Acidic | Trimeresurus gracilis | AY764141 |
| phospholipase A₂ | cTgPLA2-I gene | Trimeresurus gramineus | D31774 |
| phospholipase A₂ | | Trimeresurus | D49388 |
| | | okinavensis | |
| phospholipase A₂ | Acidic | Trimeresurus puniceus | AY355174 |
| | | E6a | |
| phospholipase A₂ | | Trimeresurus puniceus | AY355173 |
| | | G6D49 | |
| phospholipase A₂ | | Trimeresurus stejnegeri | AY211934 |
| phospholipase A₂ | group XIII | Tuber borchii | AF162269 |
| phospholipase A₂ | | Urticina crassicornis | EU003992 |
| phospholipase A₂ | II | Vipera russelli | AY286006 |
| | | siamensis | |
| phospholipase A₂ | I | Vipera russelli | AY256974 |
| | | siamensis | |
| phospholipase A₂ | group IVA, cytosolic, calcium- | Xenopus laevis | BC056041 |
| | dependent | | |
| phospholipase A₂ | group 6, cytosolic, calcium- | Xenopus tropicalis | BC123949 |
| | independent | | |
| phospholipase A₂ | group IVB, cytosolic | Xenopus tropicalis | BC087993 |
| phospholipase C | phospholipase C gamma | Aedes aegypti | XM_001649088 |
| phospholipase C | | Aedes aegypti | XM_001660587 |
| phospholipase C | phospholipase C beta | Aedes aegypti | XM_001653756 |
| phospholipase C | | Aplysia californica | DQ397516 |
| phospholipase C | phosphatidylglycerol specific, | Arabidopsis thaliana | AB084296 |
| | clone: PC-PLC6 gene | | |
| phospholipase C | phospholipase C4, nonspecific, | Arabidopsis thaliana | NM_111224 |
| | NPC4 gene | | |
| phospholipase C | | Arabidopsis thaliana | NM_101237 |
| phospholipase C | ATPLC1 gene | Arabidopsis thaliana | NM_125254 |
| phospholipase C | phospholipase C-gamma | Asterina miniata | AY486068 |
| phospholipase C | Zeta | Bos taurus | BC114836 |
| phospholipase C | delta 1 | Bos taurus | BC133304 |
| phospholipase C | Beta | Caenorhabditis elegans | AF188477 |
| phospholipase C | Gamma | Chaetopterus | EF185302 |
| | | pergamentaceus | |
| phospholipase C | | Chlamydomonas | XM_001696450 |
| | | reinhardtii | |
| phospholipase C | zeta, plcz gene | Coturnix japonica | AB369537 |
| phospholipase C | plc-21 gene | D. melanogaster | M60452 |
| phospholipase C | norpA gene | D. melanogaster | J03138 |
| phospholipase C | plc-21 gene | D. melanogaster | M60453 |
| phospholipase C | beta 3, plcb3 gene | Danio rerio | EF204528 |
| phospholipase C | gamma 1, plcg1 gene | Danio rerio | AY163168 |
| phospholipase C | phosphoinositide-specific, | Dictyostelium | M95783 |
| | DdPLC gene | discoideum | |
| phospholipase C | phosphoinositide-specific, pipA | Dictyostelium | XM_629474 |
| | gene | discoideum AX4 | |
| phospholipase C | gamma D | Drosophila | D29806 |
| | | melanogaster | |
| phospholipase C | zeta, PLCZ1 gene | Gallus gallus | AY843531 |
| phospholipase C | beta isoform, PLC gene | Homarus americanus | AF128539 |
| phospholipase C | beta 2 | Homo sapiens | BT006905 |
| phospholipase C | pancreas-enriched | Homo sapiens | AF117948 |
| phospholipase C | phosphoinositide-specific, | Homo sapiens | AF190642 |
| | PLC-epsilon | | |
| phospholipase C | beta 4, PLCB4 gene | Homo sapiens | L41349 |
| phospholipase C | delta 1 | Homo sapiens | BC050382 |
| phospholipase C | | Homo sapiens | D42108 |
| phospholipase C | epsilon 1 | Homo sapiens | BC151854 |
| phospholipase C | zeta 1 | Homo sapiens | BC125067 |
| phospholipase C | | Loligo pealei | AF258528 |
| phospholipase C | phospholipase C beta | Lytechinus pictus | AY550251 |
| phospholipase C | phospholipase C beta, | Meleagris gallopavo | U49431 |
| | erythrocyte | | |
| phospholipase C | phospholipase C-delta1 | Misgurnus mizolepis | AY134493 |
| phospholipase C | beta 1 | Mus musculus | BC058710 |
| phospholipase C | delta 4 | Mus musculus | AY033991 |
| phospholipase C | beta3 | Mus musculus | U43144 |
| phospholipase C | eta1c gene | Mus musculus | AY691174 |
| phospholipase C | eta1b gene | Mus musculus | AY691173 |
| phospholipase C | eta1a gene | Mus musculus | AY691172 |
| phospholipase C | beta-1b gene | Mus musculus | U85713 |
| phospholipase C | eta 1 gene | Mus musculus | BC042549 |
| phospholipase C | delta 1 gene | Mus musculus | BC015249 |
| phospholipase C | delta 3 gene | Mus musculus | BC031392 |
| phospholipase C | phosphatidylinositol-specific, X | Mus musculus | BC039627 |
| | domain containing 1 | | |
| phospholipase C | beta 3 | Mus musculus | BC035928 |
| phospholipase C | PLC-L2 gene | Mus musculus | AB033615 |
| phospholipase C | delta 4 | Mus musculus | BC066156 |
| phospholipase C | Gamma | Mus musculus | BC023877 |
| phospholipase C | gamma 1 | Mus musculus | BC065091 |
| phospholipase C | Zeta | Mus musculus | BC106768 |
| phospholipase C | Alpha | Mus musculus | M73329 |
| phospholipase C | beta 4 | Mus musculus | BC129883 |
| phospholipase C | beta-1a | Mus musculus | U85712 |
| phospholipase C | eta2, Plc-eta2 gene | Mus musculus strain | DQ176851 |
| | | C57BL/6J | |
| phospholipase C | beta 4, Plcb4 gene | Mus musculus strain ILS | AF332072 |
| phospholipase C | beta 1 | Mus musculus strain | AF498250 |
| | | ISS | |
| phospholipase C | phospholipase C2 | Nicotiana tabacum | AF223573 |
| phospholipase C | PLC3 gene | Nicotiana tabacum | EF043044 |
| phospholipase C | phosphoinositide-specific | Nicotiana tabacum | EF520286 |
| phospholipase C | phosphoinositide-specific | Oryza sativa | AF332874 |
| phospholipase C | beta 2, plcb2 gene | Oryzias latipes | AB254242 |
| phospholipase C | | Petunia inflate | DQ322461 |
| phospholipase C | ISC1 gene | Pichia stipitis CBS 6054 | XM_001385548 |
| phospholipase C | sphingomyelin/lysocholinephospholipid | Plasmodium falciparum | AF323591 |
| phospholipase C | Zeta | Rattus norvegicus | AY885259 |
| phospholipase C | delta4 | Rattus norvegicus | D50455 |
| phospholipase C | splice variant PLC-b4b gene, | Rattus norvegicus | U57836 |
| | brain | | |
| phospholipase C | delta 1, long form | Rattus norvegicus | EF089258 |
| phospholipase C | delta-4 | Rattus norvegicus | U16655 |
| phospholipase C | beta4 | Rattus norvegicus | L15556 |
| phospholipase C | delta isoform, PLCdsu gene | Strongylocentrotus | AY465426 |
| | | purpuratus | |
| phospholipase C | delta 4 | Sus scrofa | AF498759 |
| phospholipase C | PLC1 gene | Torenia fournieri | EU082202 |
| phospholipase C | PLC gene | Torenia fournieri | EF198328 |
| phospholipase C | delta 1 | Toxoplasma gondii | AY830139 |
| phospholipase C | | Watasenia scintillans | AB040460 |
| phospholipase C | gamma-1a | Xenopus laevis | BC070837 |
| phospholipase C | gamma-1b | Xenopus laevis | BC068831 |
| phospholipase C | gamma-1, XPLCG1a gene | Xenopus laevis | AB287408 |
| phospholipase C | PLC gene | Zea mays | AY536525 |
| phospholipase D | | Aedes aegypti | XM_001654711 |
| phospholipase D | AtPLDdelta gene | Arabidopsis thaliana | AB031047 |
| phospholipase D | PLDbeta gene | Arabidopsis thaliana | U84568 |
| phospholipase D | phospholipase D alpha 1, | Arachis hypogaea | AB232321 |
| | plda1 gene | | |
| phospholipase D | PLD gene | Arachis hypogaea | AY274834 |
| phospholipase D | phospholipase D1, | Bos taurus | BC150123 |
| | phosphatidylcholine-specific | | |
| phospholipase D | phosphatidylinositolglycan- | Bos taurus | M60804 |
| | specific | | |
| phospholipase D | N-acyl- | Bos taurus | BT021908 |
| | phosphatidylethanolamine- | | |
| | hydrolyzing, NAPE-PLD gene | | |
| phospholipase D | phospholipase D2, PLD2 gene | Bos taurus | BT026202 |
| phospholipase D | phospholipase D1, PLD1 gene | Brassica oleracea | AF090445 |
| phospholipase D | phospholipase D2, PLD2 gene | Brassica oleracea | AF090444 |
| phospholipase D | PLD gene | Caenorhabditis elegans | AB028889 |
| phospholipase D | PLD1 gene, phospholipase D1 | Cricetulus griseus | U94995 |
| phospholipase D | PLDa1 gene, phospholipase D- | Cucumis melo var. | DQ267933 |
| | alpha | inodorus | |
| phospholipase D | | Cucumis sativus | EF363796 |
| phospholipase D | glycosylphosphatidylinositol, | Dictyostelium | XM_637715 |
| | pldG gene | discoideum AX4 | |
| phospholipase D | phospholipase D3 gene | Dictyostelium | XM_632022 |
| | | discoideum AX4 | |
| phospholipase D | phospholipase D1 gene | Dictyostelium | XM_635684 |
| | | discoideum AX4 | |
| phospholipase D | | Drosophila | AF228314 |
| | | melanogaster | |
| phospholipase D | pldA gene | Emericella nidulans | AB092651 |
| phospholipase D | Alpha | Fragaria × ananassa | AY758359 |
| phospholipase D | beta 1 isoform 1a | Gossypium hirsutum | AY138249 |
| phospholipase D | Alpha | Gossypium hirsutum | EF378946 |
| phospholipase D | | Gossypium hirsutum | AF159139 |
| phospholipase D | delta isoform | Gossypium hirsutum | AF544228 |
| phospholipase D | | Homo sapiens | AF035483 |
| phospholipase D | N-acyl- | Homo sapiens | BC071604 |
| | phosphatidylethanolamine- | | |
| | hydrolyzing, cDNA clone | | |
| | MGC: 87594 IMAGE: 4375696 | | |
| phospholipase D | phosphatidylcholine-specific | Homo sapiens | BC068976 |
| phospholipase D | N-acyl- | Homo sapiens | AB112352 |
| | phosphatidylethanolamine- | | |
| | hydrolyzing | | |
| phospholipase D | PLD gene | Lolium temulentum | EU293806 |
| phospholipase D | TPLD gene | Lycopersicon | AF154425 |
| | | lesculentum | |
| phospholipase D | | Mus musculus | BC068144 |
| phospholipase D | N-acyl- | Mus musculus | AB112350 |
| | phosphatidylethanolamine- | | |
| | hydrolyzing | | |
| phospholipase D | Glycosylphosphatidylinositol | Mus musculus | AY081194 |
| phospholipase D | mPLD1 gene, | Mus musculus | U87868 |
| | phosphatidylcholine-specific | | |
| | phospholipase | | |
| phospholipase D | mPLD2 gene, | Mus musculus | U87557 |
| | phosphatidylcholine-specific | | |
| | phospholipase D2 | | |
| phospholipase D | japonica cultivar-group | Oryza sativa | D73411 |
| phospholipase D | PLD1 gene | Papaver somniferum | AF451979 |
| phospholipase D | PLD2 gene | Papaver somniferum | AF451980 |
| phospholipase D | PLD gene | Paralichthys olivaceus | AY396567 |
| phospholipase D | SPO14 gene | Pichia stipitis CBS 6054 | XM_001387066 |
| phospholipase D | PBPLD gene | Pimpinella brachycarpa | U96438 |
| phospholipase D | rPLD1 gene | Rattus norvegicus | U69550 |
| phospholipase D | PLDs gene | Rattus norvegicus | AF017251 |
| phospholipase D | 1a | Rattus norvegicus | AB003170 |
| phospholipase D | 2 | Rattus norvegicus | AB003172 |
| phospholipase D | N-acyl- | Rattus norvegicus | AB112351 |
| | phosphatidylethanolamine- | | |
| | hydrolyzing | | |
| phospholipase D | 1b | Rattus norvegicus | AB003171 |
| phospholipase D | | Rattus norvegicus | AB000779 |
| phospholipase D | | Ricinus communis | L33686 |
| phospholipase D | | Vigna unguiculata | U92656 |
| phospholipase D | alpha, PLD gene | Vitis vinifera | DQ333882 |
| phospholipase D | | Zea mays | D73410 |
| phosphoinositide | | Arabidopsis thaliana | NM_001037020 |
| phospholipase C | | | |
| phosphoinositide | | Aspergillus clavatus | XM_001272056 |
| phospholipase C | | NRRL 1 | |
| phosphoinositide | | Aspergillus fumigatus | XM_746538 |
| phospholipase C | | Af293 | |
| phosphoinositide | PLC gene | Brassica napus | AF108123 |
| phospholipase C | | | |
| phosphoinositide | gamma 2 | Homo sapiens | BC007565 |
| phospholipase C | | | |
| phosphoinositide | beta 1 | Homo sapiens | BC069420 |
| phospholipase C | | | |
| phosphoinositide | | Leishmania infantum | XM_001465631 |
| phospholipase C | | JPCM5 | |
| phosphoinositide | PLC-epsilon | Mus musculus | AB076247 |
| phospholipase C | | | |
| phosphoinositide | | Neosartorya fischeri | XM_001266832 |
| phospholipase C | | NRRL 181 | |
| phosphoinositide | PpPLC2 gene | Physcomitrella patens | AB117760 |
| phospholipase C | | | |
| phosphoinositide | PLC-1 gene | Pichia stipitis CBS 6054 | XM_001383864 |
| phospholipase C | | | |
| phosphoinositide | Epsilon | Rattus norvegicus | AF323615 |
| phospholipase C | | | |
| phosphoinositide | | Toxoplasma gondii | AY304575 |
| phospholipase C | | | |
| phosphoinositide | | Trypanosoma brucei | AY157307 |
| phospholipase C | | | |
| phosphoinositide | | Vigna unguiculata | U85250 |
| phospholipase C | | | |
| phosphoinositide | beta 1 | Xenopus tropicalis | BC118793 |
| phospholipase C | | | |
| phosphoinositide | | Zea mays | EF136661 |
| phospholipase C | | | |
| phospholipase/ | GeneID: 5826212 | Chloroflexus | NC_010175 |
| carboxylesterase | | aurantiacus J-10-fl | |
| phospholipase/ | GeneID: 5452119 | Fervidobacterium | NC_009718 |
| carboxylesterase | | nodosum Rt17-B1 | |
| phospholipase/ | GeneID: 4116934 | Rubrobacter | NC_008148 |
| carboxylesterase | | xylanophilus | |
| Phospholipase | Pha2 gene, heterodimeric | Anuroctonus | EF364040 |
| | | phaiodactylus | |
| Phospholipase | | Caenorhabditis elegans | NM_061318 |
| | | C03H5.4 | |
| Phospholipase | | Caenorhabditis elegans | NM_059984 |
| | | F36A2.9a | |
| Phospholipase | | Caenorhabditis elegans | NM_068812 |
| | | R05G6.8 | |
| Phospholipase | | Caenorhabditis elegans | NM_064039 |
| | | W02B12.1 | |
| Phospholipase | serine dependent | Homo sapiens | U89386 |
| Phospholipase | PLDb1 gene | Lycopersicon | AY013255 |
| | | esculentum | |
| Phospholipase | PLDa1 gene | Lycopersicon | AY013252 |
| | | esculentum | |
| Phospholipase | | Rattus norvegicus | U03763 |
| Phospholipase | C-zeta, plcz gene | Sus scrofa | AB113581 |
| Lysophospholipase | plb1 gene | Aedes aegypti | XM_001651691 |
| lysophospholipase | | Argas monolakensis | DQ886863 |
| lysophospholipase | | Aspergillus clavatus | XM_001271762 |
| | | NRRL 1 | |
| lysophospholipase | | Aspergillus fumigatus | XM_746859 |
| | | Af293 | |
| lysophospholipase | plb1 gene | Aspergillus fumigatus | AY376592 |
| | | CBS14489 | |
| lysophospholipase | lysophospholipase 3, | Bos Taurus | BT021838 |
| | lysosomal phospholipase A2 | | |
| lysophospholipase | lysophospholipase I | Bos Taurus | BC105143 |
| lysophospholipase | PLB gene | Cavia porcellus | AF045454 |
| lysophospholipase | | Danio rerio | BC092832 |
| lysophospholipase | Plb gene | Dictyostelium | AF411829 |
| | | discoideum | |
| lysophospholipase | plbA gene | Dictyostelium | XM_637741 |
| | | discoideum AX4 | |
| lysophospholipase | | Emericella nidulans | AB193027 |
| lysophospholipase | | Emericella nidulans | AB193027 |
| lysophospholipase | | Giardia lamblia ATCC | XM_001709168 |
| | | 50803 | |
| lysophospholipase | | Homo sapiens | BC042674 |
| lysophospholipase | lysophospholipase II | Homo sapiens | BC017193 |
| lysophospholipase | LPL-I gene, lysophospholipase | Homo sapiens | AF090423 |
| lysophospholipase | LPL1 gene | Homo sapiens | AF081281 |
| lysophospholipase | lysophospholipase 3, | Homo sapiens | BC062605 |
| | lysosomal phospholipase A2 | | |
| lysophospholipase | PLB gene | Monodelphis domestica | DQ875604 |
| lysophospholipase | lysophospholipase II | Mus musculus | AB009653 |
| lysophospholipase | lysophospholipase I | Mus musculus | U89352 |
| lysophospholipase | lysophospholipase 2 | Mus musculus | BC068120 |
| lysophospholipase | lysophospholipase 1 | Mus musculus | BC013536 |
| lysophospholipase | lysophospholipase 3 | Mus musculus | BC019373 |
| lysophospholipase | | Mus musculus | BC033606 |
| lysophospholipase | | Neosartorya fischeri | XM_001266396 |
| | | NRRL 181 | |
| lysophospholipase | Plb gene | Pichia jadinii | AB114901 |
| lysophospholipase | lysophospholipase, PLB4 gene | Pichia stipitis CBS 6054 | XM_001382254 |
| lysophospholipase | PLB1 gene | Pichia stipitis CBS 6054 | XM_001383823 |
| lysophospholipase | PLB6 gene | Pichia stipitis CBS 6054 | XM_001385976 |
| lysophospholipase | 2 | Rattus norvegicus | BC070503 |
| lysophospholipase | | Rattus norvegicus | AB009372 |
| lysophospholipase | lysophospholipase II | Rattus norvegicus | AB021645 |
| lysophospholipase | 3 | Rattus norvegicus | BC098894 |
| lysophospholipase | 1 | Rattus norvegicus | BC085750 |
| lysophospholipase | | Rattus norvegicus | BC098655 |
| lysophospholipase | Liver | Rattus norvegicus | D63885 |
| lysophospholipase | | Rattus norvegicus | D63648 |
| lysophospholipase | | Schistosoma japonicum | AF091539 |
| lysophospholipase | nte1 gene | Schizosaccharomyces | NM_001023078 |
| | | pombe | |
| lysophospholipase | | Sclerotinia sclerotiorum | XM_001594173 |
| | | 1980 | |
| lysophospholipase | II | Xenopus tropicalis | BC075270 |
| sterol esterase | | Rattus norvegicus | BC072532 |
| retinyl palmitate | type 1 | Bos Taurus | BC102781 |
| esterase | | | |
| lipolytic enzyme | GeneID: 5825102 | Chloroflexus | NC_010175 |
| | | aurantiacus J-10-fl | |
| lipolytic enzyme | GeneID: 5824919 | Chloroflexus | NC_010175 |
| | | aurantiacus J-10-fl | |
| lipolytic enzyme | GeneID: 5291607 | Clostridium beijerinckii | NC_009617 |
| lipolytic enzyme | GeneID: 5744860 | Clostridium | NC_010001 |
| | | phytofermentans | |
| lipolytic enzyme | GeneID: 5743766 | Clostridium | NC_010001 |
| | | phytofermentans | |
| lipolytic enzyme | GeneID: 5452570 | Fervidobacterium | NC_009718 |
| | | nodosum Rt17-B1 | |
| lipolytic enzyme | GeneID: 4462758 | Methanosaeta | NC_008553 |
| | | thermophila PT | |
| lipolytic enzyme | GeneID: 1474583 | Methanosarcina | NC_003552 |
| | | acetivorans | |
| lipolytic enzyme | GeneID: 1475504 | Methanosarcina | NC_003552 |
| | | acetivorans | |

### Example 4

This Example is directed to the assay for active phosphoric triester hydrolase expression in cells. Routine analysis of parathion hydrolysis in whole cells is accomplished by suspending cultures in 10 milli-Molar (“mM”) Tris hydrocholoride at pH 8.0 comprising 1.0 mM sodium EDTA (“TE buffer”). Cell-free extracts are assayed using sonicated extracts in 0.5 milliLiters (“ml”) of TE buffer. The suspended cells or cell extracts are incubated with 10 microLiters (“μl”) of substrate, specifically 100 μg of parathion in 10% methanol, and p-nitrophenol production is monitored at a wavelength of 400 nm. To induce the opd gene under lac control, 1.0 μmol of isopropyl-8-D-thiogalactopyranoside (Sigma) per ml is added to the culture media.

### Example 5

This Example is directed to the preparation of an enzyme powder. In a typical preparation, a single colony of bacteria that expresses the opd gene is selected and cultured in a rich media. After growth to saturation, the cells are concentrated by centrifugation at 7000 rotations per minute (“rpm”) for 10 minutes for example. The cell pellet is then resuspended in a volatile organic solvent such as acetone one or two times to desiccate the cells and to remove a substantial portion of the water contained in the cell pellet. The pellet may then be ground or milled to a powder form. The powder may be frozen or stored at ambient conditions for future use, or may be added immediately to a surface coating formulation. Additionally, the powder may be freeze dried, combined with a cryoprotectant (e.g., cryopreservative), or a combination thereof.

### Example 6

This Example is directed to the formation of an OPH powder and latex coating. In an example of use of the powder prepared as described in Example 5, 3 mg of the milled powder was added to 3 ml of 50% glycerol. The suspension was then added to 100 ml of Olympic® premium interior flat latex paint (Olympic®, One PPG Place, Pittsburgh, Pa. 15272 USA). This paint with biomolecular composition was then used to demonstrate the activity of the paint biomolecular composition in hydrolysis of a pesticide or a nerve agent analog.

### Example 7

This Example demonstrates, in a first set of assays, a paint product as prepared in Example 6 was applied to a hard, metal surface. The surface used in the present Example was a non-galvanized steel surface that was cleaned through being degreased, and pretreated with a primer coat. A control surface was painted with the identical paint with no biomolecular composition. Paraoxon, an organophosphorus nerve gas analog was used as an indicator of enzyme activity. Paraoxon, which is colorless, is degraded to form p-nitrophenol, which is yellow in color, plus diethyl phosphate, thus giving a visual indication of enzyme activity. In multiple assays, the surface with control paint remained white, indicating no production of p-nitrophenol, and the surface painted with the paint and biomolecular composition turned yellow within minutes, indicating an active OPH enzyme in the paint. This demonstration has shown that the surface remains active for more than 65 days, which was the maximum duration of the protocol.

In a further demonstration, the surfaces were treated as described above and each surface was then treated with paraoxon, an OP insecticide. Approximately 100 flies were then placed on each surface under a plastic cover. In each procedure, within three hours, virtually all the flies on the control surface with no paint biomolecular composition were killed by the paraoxon. In contrast, approximately 5% of the flies on the enzyme comprising surface had died.

In a demonstration of enzyme stability in the paint, a series of wood dowels were dipped into the paint comprising OPH enzyme composition. The dowels were then placed in tubes containing paraoxon to indicate enzyme activity as described above. In each case, a positive yellow color was seen except in those dowels painted with no biomolecular composition as controls. The control solution remained clear in every case.

To demonstrate the shelf life of both the dry biomolecular composition and the paint with biomolecular composition, the biomolecular composition was aged from 0 to 20 days prior to mixing in the paint. The mixed paint and biomolecular composition was then also aged from 0 to 20 prior to painting individual dowels. The enzyme composition retained strong activity after 20 days aging prior to being mixed in the paint, and for 20 days after mixing the maximum time used in the assay.

### Example 8

This Example relates to a buffered enzyme. As the hydrolysis reaction that degrades nerve agents proceeds, the local pH decreases. Without being limited to any particular mechanism, it is contemplated that due to the law of mass action, or to the optimum pH of the enzyme, the reaction is slower as the pH decreases. Because this effect may prevent or inhibit some surfaces from becoming completely decontaminated, active paint formulations have been prepared that include one or more buffering agents.

In initial procedures, the following compositions were used: 10 mg enzyme powder as described in Example 5, 100 μl 0.1 M buffer, 800 μl H₂O, and 100 μl paraoxon for a 1000 μl reaction volume.

Reactions were run for 1.5 to 2 hours and both pH and product concentration were measured. The concentration of product (p-nitrophenol) is measured by absorbance at 400 nm.

Ammonium bicarbonate, both monobasic and dibasic phosphate buffers, Trizma base and five zwitterionic buffers have been used in the active paint compositions. All the buffers were effective at allowing the reaction to proceed further to completion, thus demonstrating the function of addition of a buffering agent to the active paint compositions.

### Example 9

This Example relates to a NATO demonstration of Soman detoxification using an OPH coated surface. At the Sep. 22, 2002, meeting of the NATO Army Armaments Group in Cazaux, France, painted metal surfaces were assayed with soman using standard NATO procedures and protocols. For the assays, 10 cm×10 cm metal plates primed with standard NATO specification paints were coated with paint containing OPH. Control plates plus two different versions of the OPH enzyme composition differing in soman detoxification specificity were used. These surfaces were allowed to dry for several hours at room temperature and then assayed according to standard NATO assay protocol (described below), modified to account for the character of the surfaces treated with a paint comprising OPH.

The form of OPH in the biomolecular composition contains both the changes of the previously described H254R mutant and the H257L mutant, and is corresponding designated the “H254R, H257L mutant.” The H254R, H257L mutant demonstrates a several-fold enhanced rates of R-VX catalysis relative to either the H254R mutant or the H257L mutant, and a 20-fold enhancement of activity relative to wild-type OPH. This version of the OPH biomolecular composition has been assayed in paints treated with soman or R-VX, and are described below.

Following standard protocols, OPD painted surfaces were uniformly contaminated with an isopropanol solution containing the chemical warfare agent soman. The concentration of soman on each contaminated surface was 1.0 mg/cm². The contaminated plates were maintained at or slightly above room temperature (>20° C.) without any forced air-flow for various periods of time. A zero-time, 15 minutes, 30 minutes, and 45 minutes sample was taken for each control and biomolecular composition-containing plate series. To terminate the reaction and isolate residual soman on the plate surface, each plate was submerged in a container of isopropanol at the end-point and placed on a shaker to thoroughly extract any residual nerve agent. The solubilized portions were then quantified for soman. These assays showed that both the forms of OPH biomolecular composition were effective in detoxifying soman on metal surfaces. The two different OPH biomolecular compositions assayed detoxified the soman at levels over 65% and 77% after 45 minutes (Nato Army Armaments Group Project Group 31 on Non-Corrosive, Biotechnology-Based Decontaminants for CBW Agents, 2002). Additional assays with a CWA simulant indicated that had the NATO assay run for one to two hours, substantially all of the soman would have been detoxified.

### Example 10

This Example relates to a demonstration of an OPH biomolecular composition at Aberdeen Proving Ground (SBCCOM) in Aberdeen, Md. In these assays, a primed wooden stick was coated with paint containing OPH biomolecular composition. The painted sticks used were 2 milimeter (“mm”) in diameter×15 mm in length. By estimating that the paint layer was 0.25 mm thick, the resulting surface area was approximately 125 mm². After coating the stick with paint containing OPH biomolecular composition and allowing the paint to dry, the coated stick was inserted into a microfuge tube containing 100 μl of 3.24 mM Russian-VX agent in saline and 900 μl phosphate buffer at pH 8.3. The tubes containing R-VX and the painted sticks were allowed to sit overnight in a hood at room temperature. Appropriate controls were run simultaneously.

The following morning, the contents of the microfuge tubes were assayed for free thiols by the Ellman method. 10 mM DTNB [molecular weight (“MW”) 396.3] was prepared in 10 mM phosphate buffer at pH 8.0 for use as the indicator of enzyme activity. OPH paint's cleavage of R-VX releases a free thiol that reacts with DNTP to produce a colored product detectable spectrophotometrically at 405 nm. Ten μl of the microfuge tube contents, 100 μl DTNB solution and 890 μlphosphate buffer at pH 8.3 were read for thiol release at 405 nm using a Varian Carey 300 Spectrophotometer. The spectrophotometer was blanked with an unpainted stick control reaction. The molar equivalent of the R-VX hydrolyzed was determined using an extinction coefficient of 14,150 and the Beer-Lambert equation to calculate the product concentration. Results indicated that overnight exposure to OPH paint coated sticks resulted in decontamination of Russian VX from 32.4 μM in the original tube to less than 1 μM.

### Example 11

This Example relates to the NATO protocols for organophosphorus CWA decontamination, and describes a method for determining the decontamination properties of a coating, specifically paint, comprising a phosphoric triester hydrolase biomolecular composition. NATO assay requirements will be followed as closely as possible. Although actual assaying protocols among NATO countries vary somewhat, standard to all is the level of contamination. For exterior surfaces it is 10 grams per meter squared (“g/m²”). For interiors it is 1 g/m². Basic elements of NATO assaying procedures are as follows:

Coated Surface—A 10×10 cm metal plate coated with a coating that may comprise a biomolecular composition.

Contamination—Usually achieved with a multi-channel micropipette that can dispense 1 μl drops, with 100 drops per 10×10 cm metal plate.

Incubation—The plates will be placed into a sealed incubator, at 25° C. or 30° C., for a period ranging from 30 minutes to 3 hours.

Decontamination—The decontamination protocol varies according to the system being assayed. For example, spraying of decontamination solutions will last between 5 seconds to 20 seconds, depending on the pressure of the system.

Sampling—For standard solution-based decontamination, the assays will be normally prepared in a way that run-off decontaminant will be collected after it comes in contact with the plates and the CWA agent or CWA simulant. A set of plates will be removed for analysis at intervals, commonly being 15 minutes and 30 minutes. Any residual liquid on the plates will be added to the run-off. For enzyme biomolecular composition assays, the plates will be not rinsed after decontamination, although the rinse is standard with other decontaminants. This rinsate would also be collected for analysis. A set of plates without decontamination will be used as 0 minute, 15 minute, and 30 minute controls.

Analysis—The run-off liquid and rinsate will be immediately extracted with a solvent, such as, for example, chloroform, hexane, etc., known to dissolve the CWA agent or CWA simulant. The plates themselves can be subjected to two types of analysis: contact hazard and off-gas hazard. For contact hazard, the plates will be covered with an absorbent material. For example, the French government uses silica gel TLC plates, and the government of the USA uses a dental dam as the absorbent material. In either case, the absorbent material is held in place with a weight and incubated for 15 minutes to 30 minutes at 25° C. or 30° C. The absorbent will be removed and extracted with solvent. The plates will be then extracted with solvent to determine residual agent absorbed into the coating, and thus the contact hazard. If surface decontamination efficiency, specifically the amount of residual agent detectable, is the variable being assessed, the plates will be immediately extracted with solvent, eliminating the contact hazard step. All of the solvent samples will be analyzed by Gas Chromatography (“GC”) with a flame photometric detector (“FPD”) and a phosphorus filter for nerve agents. Some countries use Gas Chromatography-Mass Spectrometry (“GC-MS”) for the analysis.

### Example 12

This Example is of batch fermentation to produce OPH. Batch Culture-Rich Medium comprised 24 g/L yeast extract; 12 g/L casein hydrolysate; 4 ml/L glycerol; 2.31 g/L KH₂PO₄; 12.54 g/L K₂HPO₄; 0.24 g/L CoCl₂6H₂O; 2 g/L glucose; 0.2 ml/L PPG2000; and 100 μg/ml ampicillin.

Batch Culture-5 L scale was grown at the following conditions: 30° C.; 400-450 rpm agitation; DO controlled at 20%; uncontrolled initial pH between 6.8-6.9; 5 Lpm (1 vvm) aeration; and atmospheric pressure. Over a time period of 0 to 50 hours, the Escherichia coli strain's growth was measured by optical density at 600 nm, the specific paraoxonase activity was determined (nmol ml⁻¹ min⁻¹), the volumetric paraoxonase activity was determined (nmol ml⁻¹ min⁻¹), the pH measured over a range of pH 6 to pH 9, the agitation measured over a range of 0 rpm to 500 rpm, and the dissolved oxygen measured over a range of 0% to 100%.

Batch Culture—400 L scale was grown at the following conditions: 30° C.; 150-200 rpm agitation; DO at 0-100%; uncontrolled initial pH 6.58; 200-300 Lpm (0.5-0.75 vvm) aeration; and tank pressure at 0-10 psi. Over a time period of 0 to 30 hours, the Escherichia coli strain's growth was measured by optical density at 600 nm, the specific paraoxonase activity was determined (μmol ml⁻¹ min⁻¹), the volumetric paraoxonase activity was determined (μmol ml⁻¹ min⁻¹), the pH measured over a range of pH 6 to pH 8, the agitation measured over a range of 0 rpm to 200 rpm, the dissolved oxygen measured over a range of 0% to 100%, the aeration rate measured over a range of 0 to 300 Lpm, and the tank pressure measured over a range of 0 psi to 12 psi.

### Example 13

The following Example is of a large-scale fed-batch fermentation to produce OPH. Fed Batch Culture-Defined Medium comprised 13.3 g/L KH₂PO₄; 4 g/L (NH₄)₂SO₄; 1.7 g/L citric acid; 10 g/L glycerol; 1.2 g/L MgSO₄.7H₂O; 0.024 g/L MnCl₂.4H₂O; 2.26 mg/L CuCl₂.H₂O; 5 mg/L H₃BO₃; 4.5 mg/L Thiamine HCl; 4 mg/L Na₂MoO₄.7H₂O; 0.06 g/L Fe(III) citrate; 8.4 mg/L EDTA; 4 mg/L CoCl₂.6H₂O; 8 mg/L Zn(acetate)₂.H₂O; and 100 μg/ml ampicillin.

Feed: 500 g/L carbon source and 10 g/L MgSO₄.7H₂O. Batch Culture-5 L scale was grown at the following conditions: 30° C.; 200-1000 rpm agitation; DO controlled at 20%; pH controlled at 6.5; 5 Lpm (1 vvm) aeration; and atmospheric pressure. Feed was initiated as the 16^{th} hour, with the feed rate profile a constant rate with stepwise increments. Over a time period of 0 to 70 hours, the Escherichia coli strain's growth was measured by optical density at 600 nm, the specific paraoxonase activity was determined (μmol ml⁻¹ min⁻¹), the volumetric paraoxonase activity was determined (μmol ml⁻¹ min⁻¹), the pH measured over a range of pH 6 to pH 9, and the addition of the feed measured from 0 ml to 1000 ml.

### Example 14

It is contemplated that any described material formulation may be altered (e.g., by direct addition and/or component substitution) to incorporate the biomolecular composition. For example, many embodiments describe compositions and techniques for preparing, testing, and using a coating prepared de novo. However, it is contemplated that the biomolecular composition may be incorporated into a standard coating by direct addition, as described in Example 6. In specific aspects, it is contemplated that such added biomolecular composition may comprise 0.000001% to 85% or more, by weight or volume, of the final composition produced by a combination of a coating and the biomolecular composition.

Alternatively, it is contemplated that a previously described material formulation (e.g., a coating composition, a fungus prone composition) may be altered by partial or complete substitution (“replacement”) of one or more components (e.g., coating components), particularly a binder, a preservative (e.g., a fungistatic, a fungicide) and/or a particulate material component (e.g., a pigment, a rheological control agent, a dispersant) by a biomolecular composition (e.g., an antifungal peptidic agent, an enzyme, a cell-based particulate material). It is contemplated that 0.000001% to 100%, of a material formulation component may be substituted by a biomolecular composition. Additionally, the concentration of a biomolecular composition may exceed 100%, by weight or volume, of the substituted component. In specific aspects, a material formulation component may be substituted with a biomolecular composition equivalent to 0.000001% to 500%, of the component (e.g., by weight, or by volume). For example, to produce a coating with similar fungal resistance properties as a non-substituted formulation, it may require that 20% (e.g., 0.2 Kg) of a chemical fungicide may be replaced by 10% (e.g., 0.1 Kg) of an antifungal peptidic agent. In another exemplary formulation, to produce a coating with similar fungal resistance as a non-substituted formulation, it may require replacing 70% of a chemical fungicide (e.g., 0.7 Kg) with the equivalent of 127% (e.g., 1.27 Kg) of antifungal peptidic agent. In another example, a 70% (e.g., 7 Kg) of a dispersant may be replaced by 35% (e.g., 3.5 Kg) of the biomolecular composition to produce a coating with similar dispersion properties as a non-substituted formulation. In an additional example, 40% of a specific pigment (e.g., 4 Kg) may be replaced by the equivalent of 125% (e.g., 12.5 Kg) of the biomolecular composition to produce a coating with similar hiding power as a non-substituted formulation. The various assays described herein, or in the art in light of the present disclosures, may be used to determine the properties of a material formulation (e.g., a coating, a coating produced film) produced by direct addition and/or material formulation component substitution by the biomolecular composition.

The following is an example of an exterior gloss alkyd house paint comprising various particulate materials (e.g., silica, a shading pigment, bentonite clay) that may incorporate a biomolecular composition (e.g., an antibiological agent). This example of an exterior gloss alkyd house paint comprises a grind and a letdown. The grind comprises by weight or volume: a first alkyd 232.02 lb or 29.9 gallons; a second alkyd 154.2 lb or 20 gallons; an aliphatic solvent (e.g., duodecane) 69.55 lb or 1.7 gallons; lecithin 7.8 lb or 0.91 gallons; TiO₂ 185.25 lb or 5.43 gallons; 10 micron silica 59.59 lb or 2.7 gallons; bentonite clay 18.00 lb or 1.44 gallons; a second alkyd 97.22 lb or 12.61 gallons; a first alkyd 69.84 lb or 9.00 gallons; and mildewcide 7.8 lb or 0.82 gallons. In one embodiment, the grind comprises an antibiological agent (e.g., an antifungal peptidic agent) at an effective amount up to 7.8 lb or 0.82 gallons, and may optionally in combination with the mildewcide in aspects where all the mildewcide is not substituted with the antibiological agent. The letdown comprises by weight or volume: aliphatic solvent (e.g., dudecane) 19.50 lb or 3.00 gallons; a first drier (e.g., 12% solution cobalt) 2.00 lb or 0.23 gallons; a second drier (e.g., 18% solution Zr) 2.92 lb or 0.32 gallons; a third drier 3 (e.g., 10% solution Ca) 8.00 lb or 0.98 gallons; methyl ethyl ketoxime (Anti skinning agent) 3.22 lb or 0.42 gallons; an aliphatic solvent 9.75 lb or 1.50 gallons; and a shading pigment 0.3 lb or 0.04 gallons. In some embodiments, the particulate material of the coating formulation may be partly or fully substituted by the biomolecular composition. In other embodiments, the above formulation may be enhanced by direct addition of a biomolecular composition.

In another example, the following exterior flat latex house paint may be modified to incorporate a biomolecular composition (e.g., an antibiological agent). This example of an exterior flat latex house paint formulation, in typical order of addition, by weight or volume: water, 244.5 lb or 29.47 gallons; hydroxyethylcellulose, 3 lb or 0.34 gallons; glycols, 60 lb or 6.72 gallons; polyacrylate dispersant, 6.8 lb or 0.69 gallons; biocides, 10 lb or 1 gallons; non-ionic surfactant, 1 lb or 0.11 gallons; titanium dioxide, 225 lb or 6.75 gallons; silicate mineral, 160 lb or 7.38 gallons; calcined clay, 50 lb or 2.28 gallons; acrylic latex, @ 60%, 302.9 lb or 34.42 gallons; coalescent, 9.3 lb or 1.17 gallons; defoamers, 2 lb or 0.26 gallons; ammonium hydroxide, 2.2 lb or 0.29 gallons; 2.5% HEC solution, 76 lb or 9.12 gallons. In some embodiments, the paint comprises a biomolecular composition such as antifungal peptidic agent at an effective amount up to 10 In or 1 gallon (e.g., 1.8 lb or 0.82 gallon), and may optionally comprise the biocide in aspects were all of the biocide was not substituted by the antifungal peptidic agent. In some embodiments, the particulate material (e.g., silicate mineral, calcined clay, titanium dioxide) of this coating formulation may be partly or fully substituted by the biomolecular composition. In other embodiments, the above formulation may be enhanced by direct addition of a biomolecular composition.

It is contemplated that any such described coating formulation (e.g., a fungal-prone composition) may be modified to incorporate a biomolecular composition (e.g., an antifungal peptidic agent). Examples of described coating compositions include over 200 industrial water-borne coating formulations (e.g., air dry coatings, air dry or force air dry coatings, anti-skid of non-slip coatings, bake dry coatings, clear coatings, coil coatings, concrete coatings, dipping enamels, lacquers, primers, protective coatings, spray enamels, traffic and airfield coatings) described in “Industrial water-based paint formulations,” 1988, over 550 architectural water-borne coating formulations (e.g., exterior paints, exterior enamels, exterior coatings, interior paints, interior enamels, interior coatings, exterior/interior paints, exterior/interior enamels, exterior/interior primers, exterior/interior stains), described in “Water-based trade paint formulations,” 1988, the over 400 solvent borne coating formulations (e.g., exterior paints, exterior enamels, exterior coatings, exterior sealers, exterior fillers, exterior primers, interior paints, interior enamels, interior coatings, interior primers, exterior/interior paints, exterior/interior enamels, exterior/interior coatings, exterior/interior varnishes) described in “Solvent-based paint formulations,” 1977; and the over 1500 prepaint specialties and/or surface tolerant coatings (e.g., fillers, sealers, rust preventives, galvanizers, caulks, grouts, glazes, phosphatizers, corrosion inhibitors, neutralizers, graffiti removers, floor surfacers) described in Prepaint Specialties and Surface Tolerant Coatings, by Ernest W. Flick, Noyes Publications, 1991.

### Example 15

To provide a description that is both concise and clear, various examples of ranges have been identified herein. Any range cited herein includes any and all sub-ranges and specific values within the cited range, this example provides specific numeric values for use within any cited range that may be used for an integer, intermediate range(s), subrange(s), combinations of range(s) and individual value(s) within a cited range, including in the claims. Examples of specific values (e.g., %, kDa, ° C., μm, kg/L, Ku) that can be within a cited range include 0.000001, 0.000002, 0.000003, 0.000004, 0.000005, 0.000006, 0.000007, 0.000008, 0.000009, 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.10, 99.20, 99.30, 99.40, 99.50, 99.60, 99.70, 99.80, 99.90, 99.91, 99.92, 99.93, 99.94, 99.95, 99.96, 99.97, 99.98, 99.99, 99.999, 99.9999, 99.99999, 99.999999, 99.9999999, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5250, 5500, 5750, 6000, 6250, 6500, 6750, 7000, 7250, 7500, 7750, 8000, 8250, 8500, 8750, 9000, 9250, 9500, 9750, 10,000, 25,000, 50,000, 75,000, 100,000, 250,000, 500,000, 1,000,000, or more. Additional examples of the use of this definition to specify sub-ranges are given herein. For example, a cited range of 25,000 to 100,000 would include specific values of 50,000 and/or 75,000, as well as sub-ranges such as 25,000 to 50,000, 25,000 to 75,000, 50,000 to 100,000, 50,000 to 75,000, and/or 75,000 to 100,000. In another example, the range 875 to 1200 would include values such as 910, 930, etc. as well as sub-ranges such as 940 to 950, 890 to 1150, etc.

In embodiments wherein a value or range is denoted in exponent form, both the integer and the exponent values are included. For example, a range of 1.0×10⁻¹⁷ to 2.5×10⁻⁷, would include a description for a sub-range such as 1.24×10⁻¹⁷ to 8.7×10⁻¹¹.

However, general sub-ranges for each type of unit (e.g., %, kDa, ° C., μm, kg/L, Ku) are contemplated, as the values typically found within a particular type of unit are of a sub-range of the integers described above. For example, integers typically found within a cited percentage range, as applicable, include 0.000001% to 100%. Examples of values that can be within a cited molecular mass range in kilo Daltons (“kDa”) as applicable for many coating components include 0.50 kDa to 110 kDa. Examples of values that can be within a cited temperature range in degrees Celsius (“° C.”) as may be applicable in the arts of a polymeric material, a surface treatment (e.g., a coating), and/or a filler include −10° C. to 500° C. Examples of values that can be within a thickness range in micrometers (“μm”) as may be applicable to coating and/or film thickness upon a surface include 1 μm to 2000 μm. Examples of values that can be within a cited density range in kilograms per liter (“kg/L”) as may be applicable in the arts of a material formulation include 0.50 Kg/L to 20 kDa. Examples of values that can be within a cited shear rate range in Krebs Units (“Ku”), as may be applicable in the arts of a material formulation, include 20Ku to 300Ku.

### Example 16

It is contemplated that a biomolecular composition may also be incorporated into an elastomer. An elastomer may comprise a polymer that can undergo large, but reversible, deformations upon a relatively low physical stress. It is contemplated that an elastomer composition may incorporate a biomolecular composition, such as by preparation with the biomolecular composition and/or direct addition such as by a multi-pack composition. Elastomers (e.g., tire rubbers, polyurethane elastomers, polymers ending in an anionic diene, segmented polyerethane-urea copolymers, diene triblock polymers with styrene-alpha-methylstyrene copolymer end blocks, poly(p-methylstyrene-b-p-methylstyrene), polydimethylsiloxane-vinyl monomer block polymers, chemically modified natural rubber, polymers from hydrogenated polydienes, polyacrylic elastomers, polybutadienes, trans-polyisoprene, polyisobutene, cis-1,4-polybutadiene, polyolefin thermoplastic elastomers, block polymers, polyester thermoplastic elastomer, thermoplastic polyurethane elastomers) and techniques of elastomer synthesis and elastomer property analysis have been described, for example, in Walker, B. M., ed., Handbook of Thermoplastic Elastomers, Van Nostrand Reinhold Co., New York, 1979; Holden, G., ed., et. al., Thermoplastic Elastomers, 2^{nd} Ed., Hanser Publishers, Verlag, 1996.

### Example 17

A filler is a bulk material in a composition. For example, an extender pigments are used as a filler for coatings. In certain embodiments, a biomolecular composition may be used as a filler for various compositions. Examples of compositions that use fillers that are contemplated herein for incorporation of a biomolecular composition, include a composition comprising a polymer, thermoplastic material, a thermostat material, an elastomer, or a combination thereof. Such filler comprising materials have been described in Gerard, J. F., ed., Fillers and Filled Polymenrs-Macromolecular Symposia 169, Wiley-VCH, Verlag, 2001; Slusarski, L., ed., Fillers for the New Millenium-Macromolecular Symposia 194, Wiley-VCH, Verlag, 2003; and Landrock, A. H., Adhesives Technology Handbook, Noyes Publications, New Jersey, 1985.

### Example 18

This Example relates to the use of adhesives and sealants. For example, in some aspects, an adhesive may comprise a composition capable of holding at least two surfaces together in a strong and permanent manner. In another example, a sealant may comprise a composition capable of attaching to at least two surfaces, filling the space between them to provide a barrier and/or a protective coating (e.g., by filling gaps or making a surface nonporous). In certain embodiments, a biomolecular composition may be used as a component of an adhesive and/or a sealant, such as, for example, by direct addition, substitution of an adhesive and/or a sealant component (e.g., a particulate material), or a combination thereof.

Examples of adhesives and sealants (e.g., caulks, acrylics, elastomers, phenolic resin, epoxy, polyurethane, anarobic and structural acrylic, high-temperature polymers, water-based industrial type adhesives, water-based paper and packaging adhesives, water-based coatings, hot melt adhesives, hot melt coatings for paper and plastic, epoxy adhesives, plastisol compounds, construction adhesives, flocking adhesives, industrial adhesives, general purpose adhesives, pressure sensitive adhesives, sealants, mastics, urethanes) for various surfaces (e.g., metal, plastic, textile, paper), adhesive and sealant components (e.g., antifoams, antioxidants, extenders, fillers, pigments, flame/fire retardants, oils, polymer emulsions, preservatives, bactericides, fungicides, resins, rheological/viscosity control agents, starches, waxes, acids, aluminum silicates, antiskinning agents, calcium carbonates, catalysts, cross-linking agents, curing agents, clays, corn starch, starch derivatives, defoamers, antifoams, dispersing agents, emulsifying agents, epoxy resin diluents, lattices, polybutenes, polyvinyl acetates, preservatives, acrylic resins, epoxy resins, ester gums, ethylene/vinyl acetate resins, maleic resins, natural resins, phenolic resins, polyamide resins, polyethylene resins, polypropylene resins, polyterpene resins, powder coating resins, radiation coating resins, urethane resins, vinyl chloride resins, emulsion resins, dispersion resins, resin esters, rosins, silicas, silicon dioxide, stabilizers, surfactants/surface active agents, talcs, thickeners, thixotropic agents, waxes) techniques of preparation and assays for properties, have been described in Skeist, I., ed., Handbook of Adhesives, 3^{rd} Ed., Van Nostrand Reinhold, N.Y., 1990; Satriana, M. J. Hot Melt Adhesives: Manufacture and Applications, Noyes Data Corporation, New Jersey, 1974; Petrie, E. M., Handbook of Adhesives and Sealants, McGraw-Hill, New York, 2000; Hartshorn, S. R., ed., Structural Adhesives-Chemistry and Technology. Plenum Press, New York, 1986; Flick, E. W., Adhesive and Sealant Compound Formulations, 2^{nd} Ed., Noyes Publications, New Jersey, 1984; Flick, E., Handbook of Raw Adhesives 2^{nd} Ed., Noyes Publications, New Jersey, 1989; Flick, E., Handbook of Raw Adhesives, Noyes Publications, New Jersey, 1982; Dunning, H. R., Pressure Sensitive Adhesives-Formulations and Technology, 2^{nd} Ed., Noyes Data Corporation, New Jersey, 1977; and Flick, E. W., Construction and Structural Adhesives and Sealants, Noyes Publications, New Jersey, 1988.

### Example 19

This Example relates to the use of textiles. It is contemplated that a biomolecular composition may also be incorporated (e.g., direct addition to a formulation, incorporation as a component of a de novo formulation during preparation, etc.) into a material applied to a textile, such as, for example, a textile finish. Materials for application to a textile, textile finishes (e.g., soil-resistant finishes, stain-resistant finishes) and finish components (e.g., antioxidants, defoamers, antimicrobials, wetting agents, flame retardants, softeners, soil repellents, hand modifiers, antistatic agents, biocides, fixatives, scouring agents, dispersants, defoamers, anticracking agents, binders, stiffeners, cohesive agents, fiber lubricants, emulsifiers, antistats, yarn to hard surface lubricants) as well as assays for determining their properties are described, for example, in Johnson, K., Antistatic Compositions for Textiles and Plastics, Noyes Data Corporation, New Jersey, 1976; Rouette, H. K., Encyclopedia of Textile Finishing, Springer, Verlag, 2001; “Textile Finishing Chemicals: An Industrial Guide,” by Ernest W. Flick, Noyes Publications, 1990; “Handbook of Fiber Finish Technology,” by Philip E. Slade, Marcel Dekker, 1998; “ASTM Book of Standards, Volume 07.01 Textiles (I),” 2003; and “ASTM Book of Standards, Volume 07.02 Textiles (II),” 2003. A specific example of a textile finish is the trademark formulations of water repellent and/or oil repellent finish known as Scotchguard™ (3M Corporate Headquarters, Maplewood, Minn., U.S.A.).

### Example 20

This Example relates to the use of a wax and wax related materials (e.g., a polish, a wax related cleaning material, etc.). It is contemplated that a biomolecular composition may also be incorporated (e.g., direct addition to a formulation, incorporation as a component of a de novo formulation during preparation, etc.) into a material (e.g., a wax, a polish, etc.) applied to a surface or impregnated into another material after manufacture. Waxes, polishes, floor coverings, cleaning materials, and related formulations (e.g., natural waxes, fossil waxes, earth waxes, peat waxes, montana waxes, lignite paraffins, petroleum waxes, synthetic waxes, commercial modified, blended, and compounded waxes, emulsifiable waxes, waxy alcohols, waxy acids, metallic soaps, compounded waxes, paraffin wax compounds, ethyl cellulose and wax mixtures, compositions with resins and rubber) and methods of preparation of waxes, polishes, floor coverings, cleaning materials, and related formulations and assays for their properties have been described, for example, in Warth, A. H., “The Chemistry and Technology of Waxes,” Reinhold Publishing Corporation, New York, 1956; Bennet, H., “Industrial Waxes Volume II Compounded Waxes and Technology,” Chemical Publishing Co., New York, 1975; “Industrial Waxes Volume I Natural & Synthetic Waxes,” Chemical Publishing Co., New York, 1975; Flick, E. W., “Advanced Cleaning Product Formulations Household, Industrial, Automotive,” 1989; Flick, E. W., “Institutional and Industrial Cleaning Product Formulations,” 1985; Flick, E. W., “Household and Automotive Chemical Specialties Recent Formulations,” 1979; Flick, E. W., “Household, Automotive, and Industrial Chemical Formulations 2^{nd} Edition,” 1984; Flick, E. W., “Household and Automotive Cleaners and Polishes 3^{rd} Edition,” 1986; “Ullmann's Encyclopedia of Industrial Chemistry, Volume 28,” 1996; “Coatings Technology Handbook 2^{nd} Edition Revised and Expanded,” 2001; Sequeira, A. Jr., “Lubricant Base Oil and Wax Processing,” 1994; “ASTM Book of Standards, Volume 15.04 Soaps and Other Detergents; Polishes; Leather; Resilient Floor Coverings,” 2003; “ASTM Book of Standards, Volume 05.01 Petroleums and Lubricants (I),” 2003; “ASTM Book of Standards, Volume 05.02 Petroleums and Lubricants (II),” 2003; and “ASTM Book of Standards, Volume 05.03 Petroleums and Lubricants (III),” 2003.

### Example 21

This Example relates an additional embodiment where it is contemplated that the following organisms produce an OPAA that may be used in a biomolecular composition: Acinetobacter calcoaceticus ATCC 19606, Aeromonas hydrophila ATCC 7966, Aeromonas proteolytica, Arm. A isolate 1, Arm. A isolate 2, Bacillus subtilis (fr. Zuberer), Bacillus subtilis, ATCC 18685, Bacillus subtilis BRB41, Bacillus subtilis Q, Bacillus thuringensis (fr. Zuberer), Burkholderia cepacia LB400, Burkholderia cepacia T, Citrobacter diversus, Citrobacter freundii ATCC 8090, Edwardsiella tarda ATCC 15947, Enterobacter aerogenes ATCC 13048, Enterobacter cloacae 96-3, Enterobacter liquefaciens 363, Enterobacter liquefaciens 670, Erwinia carotovora EC189-67, Erwinia herbicola, Erwinia herbicola (agglomerans), Escherichia coli E63, Hafnia alvei ATCC 13337, Klebsiella pneumoniae ATCC 13883, Lactobacillus casei 686, Lactococcus lactis subsp. lactis plL253, Proteus morganaii, Proteus vulgaris ATCC 13315, Pseudomonas aeriginosa ATCC 10145, Pseudomonas aeriginosa ATCC 27853, Pseudomonas flourescens, Pseudomonas putida ATCC 18633, Pseudomonas putida PpY101, Pseudomonas sp. P, Salmonella typhimurium ATCC 14028, Serratia marcescens ATCC 8100, Serratia marcescens HY, Serratia marcescens Nima, Shigella flexneri ATCC 12022, Shigella sonnei ATCC 25931, Staphylococcus aureus ATCC 25923, Staphylococcus sp. S, Streptococcus faecalis ATCC 19433, Vibrio parahaemolyticus TAMU 109, Yersinia enterocolitica ATCC 9610, Yersinia enterocolitica TAMU 84, Yersinia frederiksenii TAMU 91, Yersinia intermedia ATCC 29909, Yersinia intermedii TAMU 86, Yersinia Kristensenia ATCC 33640, Yersinia kristensenia TAMU 95, Yersinia sp. ATCC 29912, Vibrio proteolyticus ATCC 15338, Thermus sp. ATCC 31674, Streptomyces cinnamonensis subsp. Proteolyticus ATCC 19893, Deinococcus proteolyticus ATCC 35074, Clostridium proteolyticum ATCC 49002, Aeromonas jandaei ATCC 49568, Aeromonas veronii biogroup sobria ATCC 9071, Pseudoaltermonas haloplanktis ATCC 23821, Xanthomonas campestris ATCC 33913, Pseudoalteromonas espejiana ATCC 27025, Shewanella putrefasciens ATCC 8071, Stenotrophomonas maltophilus ATCC 13637, Ochrobactrum anthropi ATCC 19286, Desulfovibrio vulgaris, or a combination thereof.

### Example 22

This Example describes assay procedure for quantitative assessment of surface activity of a composition comprising a biomolecular composition using medicine sticks/dowels. The equipment used is a U.V. Spectrophotometer, a U.V. 1 cm pathlength cuvettes, 3 ml and 100 μl volume, and 1.5 ml eppendorf tubes. The reagents used include paraoxon (MW 275.21, ChemService cat#PS-610), 99% CHES (“2-[cyclohexylamino]ethanesulfonic acid”), (MW 207.3, Sigma cat #C-2880), and CoCL₂ 6H₂O (MW 237.9, Sigma cat #C-3169). 1 M CoCl₂, sterile, can be prepared as 23.79 g CoCl₂ per 100 ml ddH₂0 that is filter sterilized or autoclaved. 200 mM CHES, pH 9.0, sterile can be prepared as 4.15 g+80 ml ddH₂O, pH to 9.0 with NaOH, where the total volume with ddH₂O is 100 ml, and can be filter sterilized or autoclaved. The assay buffer is 20 mM CHES, pH 9.0, 50 μM CoCl₂.

In a 1.5 mL Eppendorf tube add: paraoxon to 1 mM (ex: 126 μl of 12 mM paraoxon) and assay buffer to 1.5 ml (ex: 1374 μl CHES buffer). Add a 5 mm length of treated stick to start the reaction, mix by inverting. Take 10 μl samples at 1 minute intervals, diluting with 90 μL CHES buffer into a 100 μl cuvette. Record the absorbance at 400 nm (A₄₀₀ₙₘ), blanking against CHES buffer+paraoxon. A small amount of hydrolysis of paraoxon without biomolecular composition may occur. Mix by inversion before each time point.

Alternatively, in a 3 ml cuvette, add: paraoxon to 1 mM (ex: 168 μl of 12 mM paraoxon), and assay buffer to 2.0 ml (ex: 1832 μl CHES buffer). Add a 5 (or 15 mm) length of treated stick to start the reaction. Record the (A₄₀₀ₙₘ) at the following time points: 0, 15, 30, 45, 60, 120, 180, 240, 300, 360, 420 and 480 minutes. Mix by inversion at regular intervals. If absorbencies above 2.5 are observed, dilute 10 μL samples with 90 μL CHES buffer in a 100 μL cuvette.

The following results demonstrate 90% degradation of the paraoxon over the time frame of measurement by a paroxonase bimolecular additive as determined by the dowel assay.

TABLE 18
Results Paroxonase Degradation

| Time | Replicates | | | | |
|---|---|---|---|---|---|
| (seconds) | A | B | C | umoles p-NP | Std Dev |
| 0 | 0.0218 | 0.0218 | 0.0224 | 0.0220 | 0.0003 |
| 120 | 0.1794 | 0.1518 | 0.1253 | 0.1522 | 0.0271 |
| 240 | 0.4359 | 0.3953 | 0.3418 | 0.3910 | 0.0472 |
| 360 | 0.7529 | 0.6541 | 0.6218 | 0.6763 | 0.0683 |
| 480 | 0.9494 | 0.8971 | 0.8894 | 0.9120 | 0.0327 |
| 600 | 0.9724 | 0.9688 | 0.9659 | 0.9690 | 0.0032 |
| 720 | 0.9706 | 0.9706 | 0.9729 | 0.9714 | 0.0014 |
| 840 | 0.9700 | 0.9694 | 0.9782 | 0.9725 | 0.0049 |
| 960 | 0.9535 | 0.9535 | 0.9435 | 0.9502 | 0.0058 |
| 1080 | 0.9600 | 0.9935 | 0.9912 | 0.9816 | 0.0187 |
| 1200 | 0.9500 | 0.9665 | 0.9682 | 0.9616 | 0.0101 |

p-NP = reaction product

### Example 23

This example demonstrates the production of a biomolecular composition by fed-batch fermentation at 200L scale manufacture. The production timeline is as follows:

TABLE 19
Production Timeline

| Day | Time | Operation | Comments |
|---|---|---|---|
| 2-4 weeks | NA | Order supplies | Ensure that all reagents and supplies |
| before day 1 | | | are ready for use |
| 1-30 days | NA | Make trace | Make sufficient trace element |
| before day 1 | | element solutions | solutions for all fermentations and |
| | | | seeds |
| 2-5 days | NA | Plasmid | The transformation may be |
| before day | | transformation into | completed with sufficient time for the |
| | | host strain | agar plate to develop discrete |
| | | | colonies before it is used to inoculate |
| | | | seed cultures. |
| 2-14 days | NA | Make shake flasks | At least 2 × 50 ml and 2 × 1 L flasks are |
| before day 1 | | for seed cultures | required |
| 2-14 days | NA | Make antibiotic for | At least 2.5 ml of 10% antibiotic |
| before day 1 | | seed cultures | solution is required |
| Day 1 | 09:00 | Pre-seed culture | Inoculation of pre-seed culture flasks |
| Day 1 | 18:00 | Seed culture | Inoculation of seed culture flasks |
| Day 1 | 10:00 | Fermentor set up | Prepare base medium and |
| | | | fermentor, sterilize |
| Day 1 | 11:00 | Prepare feed | Prepare and sterilize solutions. After |
| | | solution and other | sterilization, store, add or attach |
| | | additions | solutions as appropriate |
| Day 2 | 09:30 | Prepare for | Get fermentor and all peripheral |
| | | inoculation | items ready for inoculation |
| Day 2 | 10:00 | Inoculate | Add 2 L of inoculum to fermentor |
| | | Fermentor | |
| Day 3 | 10:00-20:00 | Start feed | Start nutrient feed when initial |
| | | | glucose has been exhausted |
| Days 3-5 | | Monitor fermentor | Adjust feed rates, add cobalt chloride |
| Day 4 | 14:00 | Set up filtration | Prepare filtration system for next day |
| | | system | |
| Day 5 | 09:00 | Harvest | Diafilter with water, then concentrate |
| | | | cells |
| Day 5 | 14:00 | Package and ship | Package the concentrated cells in |
| | | | 20 L carboys or a 30 gallon drum and |
| | | | ship to Aero-Instant |
| Day 5 | 15:00 | Cleaning | Clean fermentor and filter |
| Day 5 | 14:00 | OPD assays | Do paraoxonase assays on |
| | | | fermentation and harvest samples |

The reagents and supplies used are as follows:

TABLE 20
Reagents and Supplies Required

| Chemical | Supplier | Amount | |
|---|---|---|---|
| Yeast extract | USB | 30 | g |
| Tryptone | Difco | 30 | g |
| NaCl | Baker | 30 | g |
| Ampicillin | USB | 30 | g |
| KH₂PO₄ | Baker | 2.2 | kg |
| (NH₄)₂SO₄ | Baker | 0.7 | kg |
| Citric acid | Baker | 0.3 | kg |
| Antifoam 204 | Sigma | 250 | ml |
| CoCl₂•6H₂O | Fisher/sigma | 100 | g |
| CuCl₂•H₂O | Baker | 1 | g |
| H₃BO₃ | Baker | 2 | g |
| Na₂MoO₄ | Baker | 2 | g |
| Fe(III) citrate | Aldrich | 25 | g |
| EDTA | Baker | 5 | g |
| Glucose | USB/Pfanstiehl | 3.5 | kg |
| MgSO₄•7H₂O | Baker | 0.7 | kg |
| Thiamine•HCl | Sigma | 35 | g |
| NH₄OH | Fisher | 10 | L |
| Glycerol | Fisher | 20 | L |
| Paraoxon | | | |

TABLE 21
Supplies

| Item | Supplier | Amount |
|---|---|---|
| Sterile loops | Fisher | 1 pack |
| Erlenmeyer flasks | Fisher | 3 × 250 ml; 2 × 2 L |
| Nalgene 250 ml filter | Fisher | 5 |
| housings | | |
| Nalgene 500 ml filter | Fisher | 2 |
| housings | | |
| Size 16 silicone tubing | Fisher | 1 reel |
| Size 25 silicone tubing | Fisher | 1 reel |
| 5 m² Optisep 11,000 PS | NCSRT | 2 |
| filters, 0.5 μm | | |

Plasmid Transformation into Host strain: Transformation Day 1, do as follows: Purified OPD-RL plasmid is stored at −20° C. in a bioexpression and fermentatation facility (“BFF”) BioXpress −20° C. freezer. Remove the relevant vial(s) and thaw. Transform into E. coli DH5α(Invitrogen). Add 2 μl of plasmids to 200 μl Invitrogen DH5a competent cells. Incubate cells on ice for 25 minutes. Heat shock the cells in a water bath at 42° C. for 30 seconds, then return to the ice for 2 minutes. Aseptically add 500 μl sterile SOB (SOB: 900 ml of distilled H₂O, 20 g Bacto Tryptone, 5 g Bacto Yeast Extract, 2 ml of 5M NaCl, 2.5 ml of 1M KCl, 10 ml of 1M MgCl₂, 10 ml of 1M MgSO₄, 1L with distilled H₂O). Incubate for 60 minutes at 37° C. Plate 650 μl and 50 μl of the cells in SOB medium onto LB agar with ampicillin (100 μg/ml). Spread for single colonies and incubate at 37° C. overnight. Transformation Day 2, do as follows: Remove the plates from the incubator. Store at 4° C.

Seed Production: LB Medium for Seed cultures as follows: LB medium is made in standard batches. The recipe used is as follows: 10 g/L tryptone (Difco); 10 g/L NaCl (Baker); and 10 g/L yeast extract (Difco).

Day 1, at 09:00—pre-seed the culture growth as follows: At approximately 08.30, turn on the laminar flow hood, swab with ethanol, and switch on the UV light for 10 minutes. Select 2×250 ml LB flasks each containing 50 ml of LB medium. Record the batch and chemical lot numbers of the materials that are used. Aseptically add 50 μl of 10% ampicillin stock solution to each flask, and attach a copy of the recorded material information. At 09.00, aseptically pick several colonies from the plate and resuspend in sterile medium. Incubate the flasks at 30° C. and 250 rpm in a New Brunswick Scientific Series 15 incubator/shaker for 9 h.

Day 1, at 17:30, do as follows: Remove 10 μl of culture and check microscopically to confirm that there is no contamination. If the cultures pass the microscopic examination proceed to the next seed stage. Turn on the laminar flow hood, swab with ethanol, and switch on the UV light for 10 minutes. Select two 2 L LB flasks each containing 1 L of LB medium. Attach a copy of record of the batch number and chemical lot numbers of the materials used. Aseptically add 1 ml of 10% ampicillin stock solution to each flask. Attach a copy of a record of the batch number and chemical lot numbers to the materials used. At 18.00, aseptically transfer 10-20 ml of the 50 ml pre-seed culture to each of the 2L flasks. Incubate the flasks at 30° C. and 250 rpm in a New Brunswick Scientific Series 15 incubator/shaker overnight. Record all information regarding times and date of procedure, materials used, personel conducting the work, and reaction conditions, and attach a copy to the other recorded information.

Fermentor set up was as follows: Production is done at 200L scale. The approximate volumes break down as follows: 160 L batch medium; 2 L seed cultures; 30-40 L feed solution; 5-7 L base addition; 1-3 L sample removal; to produce a total volume of about 200L.

The fermentor used is a WB Moore, Inc. 250L stainless steel fermentor equipped with an Allen Bradley PLC controller. Temperature, pH, agitation, aeration, pressure and oxygen addition are controlled. Dissolved oxygen is measured and controlled by a sequential cascade of agitation rate, aeration rate, pressure, and oxygen supplementation.

Day 1, 10:00, Prepare the fermentor as follows: Calibrate the pH probe. Check the DO probe. Replace the electrolyte and membrane if useful. Insert the pH probe and DO probes. Add approximately 100L of DI water to the tank. Prepare the base medium. The following components are added to the fermentor prior to sterilization.

TABLE 22
Materials to be Added to Fermentor

| Chemical | Manufacturer | Amount required | |
|---|---|---|---|
| KH₂PO₄ | Baker | 2128 | g |
| (NH₄)₂SO₄ | Baker | 640 | g |
| Citric acid | Baker | 272 | g |
| Trace element | BFF | 160 | ml |
| solution A | | | |
| Trace element | BFF | 1600 | ml |
| solution B | | | |
| Antifoam 204 | Sigma | 20 | ml |
| Water | | QS to 155 | L |

Sterilize the tank at 122° C. for one hour. Cool the tank to 30° C. and set the control temperature. Record all information regarding times and date of procedure, materials used, personel conducting the work, and reaction conditions, and attach a copy to the other recorded information. Prepare the medium additions as follows.

TABLE 23
Trace Element Solution A

| Chemical | Manufacturer | Amount required | |
|---|---|---|---|
| Citric acid | Baker | 2.5 | g |
| CoCl₂•6H₂O | Fisher/sigma | 1.0 | g |
| CuCl₂•H₂O | Baker | 0.57 | g |
| H₃BO₃ | Baker | 1.25 | g |
| Na₂MoO₄ | Baker | 1.0 | g |
| DI water | | QS to 500 | ml |

Store at 4° C. until use. Record all information regarding times and date of procedure, materials used, personel conducting the work, and reaction conditions, and attach a copy to the other recorded information.

TABLE 24
Trace Element Solution B

| Chemical | Manufacturer | Amount required | |
|---|---|---|---|
| Fe(III) citrate | Aldrich | 24 | g |
| EDTA | Baker | 3.36 | g |
| DI water | | QS to 4 | L |

Store at 4° C. until use. Record all information regarding times and date of procedure, materials used, personel conducting the work, and reaction conditions, and attach a copy to the other recorded information.

TABLE 25
Glucose Addition Solution

| Chemical | Manufacturer | Amount required | |
|---|---|---|---|
| Glucose | USB/Pfanstiehl | 3200 | g |
| MgSO₄•7H₂O | Baker | 192 | g |
| DI water | | QS to 6 | L |

Sterilize in an autoclave at 122° C. for one hour.

TABLE 26
Cobalt Chloride Solution

| Chemical | Manufacturer | Amount required | |
|---|---|---|---|
| CoCl₂ | Fisher/Sigma | 54.9 | g |
| DI water | | QS to 500 | ml |

Filter sterilize in two 250 ml aliquots using Nalgene 0.22 μm filter units.

TABLE 27
Thiamine Solution

| Chemical | Manufacturer | Amount required | |
|---|---|---|---|
| Thiamine•HCl | Sigma | 33.7 | g |
| DI water | | QS to 100 | ml |

Note: 2 ml of this solution will be added to the fermentor.

TABLE 28
Ampicillin Solution

| Chemical | Manufacturer | Amount required | |
|---|---|---|---|
| Ampicillin, sodium salt | USB | 20 | g |
| DI water | | QS to 250 | ml |

Filter sterilize in using a Nalgene 0.22 μm filter unit.

TABLE 29
Base Solution

| Chemical | Amount required |
|---|---|
| Aqueous NH₄OH | 7.5 L |

Sterilize an empty reservoir bottle at 122° C. for 30 minutes. When cool, empty three 2.5 L ammonium hydroxide bottles into the reservoir. Use extreme caution and wear protective clothing.

TABLE 30
Feed Solution

| Chemical | Amount required | |
|---|---|---|
| Glycerol | 20 | L |
| MgSO₄•7H₂O | 400 | g |
| DI water | QS to 40 | L |

Make up in reservoir tank fitted out for feeding the fermentor, with silicone tubing capable of feed rates of 2-40 ml/min. Sterilize the tank at 122° C. for one hour.

Fermentor Operations on, Day 2, 09:30, include: making additions to the Fermentor, adding the following solutions, in order:

TABLE 31
Fermentor Solution

| Addition | Amount | |
|---|---|---|
| Ampicillin solution | 250 | ml |
| Glucose/MgSO4 solution | 6 | L |
| Thiamine solution | 2 | ml |
| Cobalt chloride solution | 250 | ml |

With the feed bottle on the balance of the Scilog pump system, attach the feed reservoir to the feed port on the fermentor. Run the tubing through the scilog pump and prime the lines. With the base reservoir on the Ohaus balance, attach to the base port on the fermentor. Run the tubing through a peristaltic pump and prime the lines. Plug the pump into the base socket on the rear of the fermentor. Take a sample from the fermentor. Store a portion in a labeled sterile falcon tube. Check the pH of another portion offline. Adjust the pH calibration if useful. Calibrate the dissolved oxygen probe. Check and set all fermentation parameters.

TABLE 32
Fermentation Parameters

| Parameter | Set point |
|---|---|
| Temperature | 30° C. |
| pH | 6.5 |
| Dissolved oxygen | 60 mBar (30%) |
| Air flow rate | 50-200 LPM |
| Agitation Rate | 100-350 rpm |
| Oxygen flow rate | 50 LPM (on demand) |
| Tank pressure | 0-5 psi |

Remove the seed culture flasks from the shaker and take 10 μl of culture to check microscopically to confirm that there is no contamination. Also check the OD₆₀₀ of the cultures. If the cultures pass the microscopic examination proceed to the next seed stage. Record all information regarding times and date of procedure, materials used, personel conducting the work, and reaction conditions, and attach a copy to the other recorded information.

Day 2 10:00, inoculation, do as follows: Add the entire contents of the two seed culture flasks to the 250L fermentor. From the harvest port, take a 20-50 ml sample. Measure the optical density at 600 nm. Using a Boehringer glucose analyzer, measure the glucose concentration of the medium. Read from the controller on the fermentor and the attached balances. Record all information regarding times and date of procedure, materials used, personel conducting the work, and reaction conditions, and attach a copy to the other recorded information. Every 2-4 hours, take samples and process as described above. Record all information regarding times and date of procedure, materials used, personel conducting the work, and reaction conditions, and attach a copy to the other recorded information.

Days 3-5, Start Feed as follows: When the glucose level is below 2 g/L start the feed pump. The glucose may be reduced to this level at between 24-36 hours after inoculation. At this point the sampling frequency may be reduced to 3-5 times per day.

Feed Profile is a follows: Program the following feed profile into the Scilog pump. Execute the program at the start of feeding.

TABLE 33
Feed Profile.

| Time | Feed Rate (ml/min) | Cumulative feed added (L) |
|---|---|---|
| Feed start | 4 | 0 |
| Feed start + 2 h | 6 | 0.48 |
| Feed start + 4 h | 8 | 1.20 |
| Feed start + 6 h | 10 | 2.16 |
| Feed start + 8 h | 12 | 3.36 |
| Feed start + 10 h | 16 | 4.80 |
| Feed start + 36.67 h | 0 | 40.00 |

Samples for paraoxonase assays are as follows: From this point in the fermentation, when samples are taken, centrifuge 2×1 ml samples in eppendorf tubes and store the cells at −80° C. until testing for paraoxonase activity.

Cobalt chloride addition is as follows: When the OD₆₀₀ attains a level of 40±10, add the remaining cobalt chloride.

Fermentation Completion is as follows: The fermentation is complete when (1) the cells stop growing, as indicated by a combination of a drop in OD₆₀₀, a drop in oxygen demand and an increase in pH; (2) the feed is exhausted; (3) the elapsed fermentation time reaches 72 h. At the completion of the fermentation, turn off the feed pump and the base pump. Cool the reactor to <15° C. Note the condition of the culture at this time, as foaming is sometimes observed as the culture stops growing and is cooled. Take one or more sample from the fermentor and measure the average wet weight of the culture Harvesting, Day 4, 14:00 is as follows: Set up the NCSRT filtration system. Use two 5 m² Optisep 11,000 polysulfone filters, 0.05 μm pore size, 0.875 mm channel height. Rinse the system with at least 200 L of DI water.

Day 5, 08:00 is as follows: Fill a reservoir tank with 600L of DI water. When the fermentation is complete and the culture has been cooled to <15° C., hook up the filtration system to the tank as follows: Release pressure from the tank and stop agitation. Attach the pump inlet to the fermentor drain. Place the filtration system return in the top of the fermentor. Connect the water reservoir to the feed inlet. Open the fermentor drain valve. Attach a line to the sample port to estimate culture volume. Estimate and record culture volume. Estimate and record cell mass in the fermentor. Keep a sample for paraoxonase assay.

Start filtration as follows: Start the filtration system pump at a low flow rate. As the system is filled, gradually increase the pump rate until the flow rate across the membrane is 300 L/min, or until the pressure at the bottom of the membrane is 10 psi, whichever comes first. Do not allow the membrane pressure to exceed 11 psi. Record all information regarding times and date of procedure, materials used, filtration data, personel conducting the work, and reaction conditions, and attach a copy to the other recorded information. Measure and record the initial flux rate (L/min). Check that the filtrate is clear and that product is not crossing the membrane. If the filtrate is slightly cloudy reduce the flow rate and then recheck. Start adding DI water to the fermentor at a rate equal to the flux rate to maintain the culture volume. Diafilter with three volumes (600 L) of DI water, noting the time at which diafiltration is complete.

When diafiltration is complete, continue filtering as before, to concentrate the washed culture. Monitor the membrane pressure, and reduce the pump rate is the pressure rises. Continue concentration until the cell density attains a level of 700±100 g/L or until the pump rate is too low to continue. Without shutting off the pump, open the system drain line and pump the product into 20L carboys. Take one or more sample of the final product and measure the wet weight, and average the wet weight. Measure the final product volume, and estimate the cell mass in product. Save a sample for a paraoxonase assay. Label the carboys and store at 4° C. ready for shipping.

Downstream Processing is as follows: The product is ready for spray drying applications. It may be shipped to other facilities on 20L carboys can be shipped with ice packs.

Cleaning is as follows: Clean the fermentor and filter system thoroughly.

The paraoxonase assay is as follows: This describes assaying of biomolecular composition for paraoxonase activity in a 96-well plate using a plate reader. The equipment and reagents used are shown on the table below.

TABLE 34
Equipment and Reagents

Equipment
Plate Reader
Reagents
Paraoxon (MW 275.21, ChemService cat#PS-610)
CHES (2-[cyclohexylamino]ethanesulfonic acid), 99%
(MW 207.3, Sigma cat# C-2880)

Sample preparation is as follows: paraoxon is prepared in the disclosures herein or by the techniques of the art; 200 mM CHES, pH 9.0, sterile is prepared by adding 4.15 g and 80 mL ddH₂O, adjusting to pH 9.0 with NaOH, bringing to 100 mL total volume with ddH₂O, and filter sterilizing or autoclaving; and working solutions prepared by diluting 200 mM CHES to 20 mM and 40 mM.

Plate Reader Assay is as follows: weighing approximately 15 mg of wet cell biomass (or dried additive) in a 1.5 mL Eppendorf tube; resuspending in appropriate volume 20 mM CHES to make 30 mg/mL suspension; prepare a serial dilution of this solution as 1:2, 1:5, and 1:10; loading 2 uL of each dilution in triplicate in the 96-well plate (i.e., wells 1-3 will have undiluted solution, 4-6 will all have 1:2, 7-9 will be 1:5 and 10-12 will be 1:10); adding 39.36 uL MilliQ H₂O to each of the wells; adding 50 uL 40 mM CHES to each well; adding 10.64 uL of 9.4 mM Paraoxon is added to each well; setting the kinetic protocol to read absorbance at 405 nm taking 50 readings, at 7 second intervals; and determining maximum velocity for analysis using usually at least 20 points.

Record personnel involved in the procedures implemented. Quality control and safety procedures were as described in Example 33, including use of a hood for material handling as occurred.

### Example 24

This Example demonstrates the harvesting of a biomolecular composition produced by fermentation.

Harvesting is as follows: Set up the NCSRT filtration system. Use two 5 m² Optisep 11,000 polysulfone filters, 0.05 μm pore size, 0.875 mm channel height. Rinse the system with at least 200 L of DI water. Fill a reservoir tank with 600 L of 100 mM sodium bicarbonate.

When the fermentation is complete and the culture has been cooled to <15° C., hook up the filtration system to the tank as follows: release pressure from the tank and stop agitation. Attach the pump inlet to the fermentor drain. Place the filtration system return in the top of the fermentor. Connect the water reservoir to the feed inlet. Open the fermentor drain valve. Attach a line to the sample port to estimate culture volume, and estimate the culture volume, cell mass in the fermentor, and keep a sample for the paraoxonase assay.

Start filtration as follows: Start the filtration system pump at a low flow rate. As the system is filled, gradually increase the pump rate until the flow rate across the membrane is 300 L/min, or until the pressure at the bottom of the membrane is 10 psi, whichever comes first. Do not allow the membrane pressure to exceed 11 psi. Record all information regarding times and date of procedure, materials used, filtration data, personel conducting the work, and reaction conditions, and attach a copy to the other recorded information. Measure the initial flux rate (L/min). Check that the filtrate is clear and that product is not crossing the membrane. If the filtrate is slightly cloudy reduce the flow rate and then recheck. Start adding 100 mM sodium bicarbonate to the fermentor at a rate equal to the flux rate to maintain the culture volume. Diafilter with three volumes (600 L) of 100 mM sodium bicarbonate, and record the time at which diafiltration is complete.

When diafiltration is complete, continue filtering as before, to concentrate the washed culture.

Monitor the membrane pressure, and reduce the pump rate is the pressure rises. Continue concentration until the cell density attains a level of 700±100 g/L or until the pump rate is too low to continue. Without shutting off the pump, open the system drain line and pump the product into 20 L carboys. Take one or more sample of the final product and measure the wet weight, and determine the average wet weight, measure the final product volume, estimate the cell mass in the product, and keep a sample for a paraoxonase assay. Label carboys and store at 4° C. ready for shipping to other faculties or end users.

### Example 25

This Example demsonstrates the preparation and chararcterization of the organophosphourus compound and OPH substrate, paraoxon for use in various other examples and assays described herein.

The equipment used is as follows: a U.V. Spectrophotometer, U.V. 1 cm pathlength cuvettes, and a stir plate.

The reagents used are as follows: Paraoxon, 200 mg (Chem Service, cat #PS-610, MW 275.21, ∈₂₇₄=8.9×10³)

Samples are prepared as follows: add 200 mgs of paraoxon, which should be as an oily liquid in 100 mg aliquots, to 50 mls ddH₂O; and letting stir in the cold for 2-3 days to be sure it is fully dispersed and dissolved, though as the paraoxon should be 14.5 mM; due to loss during pipetting, solubility, etc., the solution rarely reaches this concentration.

The analysis of samples should be conducted as follows: To determine the [paraoxon], make the following dilutions—1:100 with 10 μl paraoxon stock:990 μl ddH₂O, 1:500 with 2 μl paraoxon stock:998 μl ddH₂O, and 1:1000 with 10 μl (1:100) paraoxon:990 μl ddH₂O; read O.D. at 274 nm; with typical readings being—1:100=1, 1:500=0, and 1:1000=0. The extinction coefficient of diethyl p-nitrophenyl phosphate (paraoxon) is 8,900 M⁻¹ cm⁻¹, and the sample calculations are as follows: (1.1/8,900)*100=0.0123 μmol/μl* (0.0123 μmol/μl)*(1,000,000 μl/l)*(mm/1000 μmoles)=12.3 mM concentration of paraoxon.

Procedural cautions: Make sure pipette tips fit the pipette. Check the liquid level in the tips for air bubbles, etc., particularly when using the multichannel pipettes. Quality control and safety procedures were as described in Example 33. Quality control included operating, maintaining, and maintenance of all equipment in accordance with normal practice of the art and any manuals provided from the manufacturer, and maintenance records kept; using correctly labeled working solutions prior to the date of expiration, and disposing of others which are out of date or prepared incorrectly; and disposing of leftover QC samples in the appropriate hazard container, and not using QC samples made one day on the next day.

### Example 26

This Example demonstrates the preparation and chararcterization of the organophosphurus compound and OPH substrate, paraoxon for use in various other examples and assays described herein.

The equipment used is as follows: a U.V. Spectrophotometer, U.V. 1 cm pathlength cuvettes, and a stir plate.

The reagents used are as follows: Paraoxon, 200 mg (Chem Service, cat #PS-610, MW 275.21, ∈274=8.9×10³)

Samples are prepared as follows: add 200 mgs of paraoxon, which should be as an oily liquid in 100 mg aliquots, to 50 mls ddH₂O; and letting stir in the cold for 2-3 days to be sure it is fully dispersed and dissolved, though as the paraoxon should be 14.5 mM; due to loss during pipetting, solubility, etc., the solution rarely reaches this concentration.

The analysis of samples should be conducted as follows: To determine the [paraoxon], make the following dilutions—1:100 with 10 μl paraoxon stock:990 μl ddH₂O, 1:500 with 2 μl paraoxon stock:998 μl ddH₂O, and 1:1000 with 10 μl (1:100) paraoxon:990 μl ddH₂O; read O.D. at 274 nm; with typical readings being—1:100=1, 1:500=0, and 1:1000=0. The extinction coefficient of diethyl p-nitrophenyl phosphate (paraoxon) is 8,900 M⁻¹ cm⁻¹, and the sample calculations are as follows: (1.1/8,900)*100=0.0123 μmol/μl* (0.0123 μmol/μl)*(1,000,000 μl/l)*(mm/1000 μmoles)=12.3 mM concentration of paraoxon.

Procedural cautions: Make sure pipette tips fit the pipette. Check the liquid level in the tips for air bubbles, etc., particularly when using the multichannel pipettes. Quality control and safety procedures were as described in Example 33. Quality control included operating, maintaining, and maintenance of all equipment in accordance with normal practice of the art and any manuals provided from the manufacturer, and maintenance records kept; using correctly labeled working solutions prior to the date of expiration, and disposing of others which are out of date or prepared incorrectly; and disposing of leftover QC samples in the appropriate hazard container, and not using QC samples made one day on the next day.

### Example 27

This Example demonstrates a lipase assay determining the efficacy of lipase in a coating (e.g., paint). Films of Sherwin-Williams Acrylic Latex comprising lipase were assayed 7 months after they were prepared. Materials used are shown in the table below.

TABLE 35
Materials

200 mM TRIS Buffer (Sigma Product # T1503); brought to
pH = 7.1 with HCl
4-nitrophenyl acetate (Sigma Product # N8130); 14.5 mM solution in
isopropyl alcohol
Lipase from porcine pancreas (Sigma Product # L3126)
2 mL microtubes
Pipette
Pipette Tips
Plate Reader
96-well Plate

The reaction procedure included: cutting 1 cm×3 cm free film coupon sizes; placing individual coupons into labeled 2 mL microtubes, with each of the coupon samples tested in triplicate; adding 750 μl 200 mM TRIS to each microtube; adding 600 ul ddH2O to each microtube; adding 150 ul 14.5 mM p-nitrophenyl acetate to each microtube; preparing control samples that had 750 ul 200 mM TRIS, 600 ul ddH2O, and 150 ul 14.5 mM p-nitrophenyl acetate; taking out at each desired time point, 100ul and reading the absorbance at 405 nm in a 96-well plate; and plotting absorbance vs. time to calculate the slope. Data and calculate values are shown below, demonstrating lipase activity in a cured coating's film 7 months after preparation.

TABLE 36
Absorbance at 405 nm Data

| Time | | | | | | | |
|---|---|---|---|---|---|---|---|
| (min) | Blank | Control | | | Lipase | | |
| 0 | 0.0423 | 0.0423 | 0.0423 | 0.0423 | 0.0423 | 0.0423 | 0.0423 |
| 15 | 0.0477 | 0.0475 | 0.0487 | 0.0495 | 0.1760 | 0.1933 | 0.1719 |
| 30 | 0.0562 | 0.0556 | 0.0550 | 0.0572 | 0.3353 | 0.3631 | 0.3137 |
| 45 | 0.0587 | 0.0598 | 0.0616 | 0.0624 | 0.4642 | 0.5084 | 0.4486 |
| 60 | 0.0643 | 0.0673 | 0.0684 | 0.0691 | 0.6008 | 0.6069 | 0.5565 |
| 90 | 0.0751 | 0.0762 | 0.0785 | 0.0783 | 0.7181 | 0.7896 | 0.7591 |
| Slope | 0.0004 | 0.0004 | 0.0004 | 0.0005 | 0.0095 | 0.0105 | 0.0091 |

TABLE 37
Average pNP Absorbance at 405 nm

| Time | Blank | Control Avg | Lipase Avg | Control SD | Lipase SD |
|---|---|---|---|---|---|
| 0 | 0.0423 | 0.0423 | 0.0423 | 0.0000 | 0.0000 |
| 15 | 0.0477 | 0.0486 | 0.1804 | 0.0010 | 0.0114 |
| 30 | 0.0562 | 0.0559 | 0.3374 | 0.0011 | 0.0248 |
| 45 | 0.0587 | 0.0613 | 0.4737 | 0.0013 | 0.0310 |
| 60 | 0.0643 | 0.0683 | 0.5881 | 0.0009 | 0.0275 |
| 90 | 0.0751 | 0.0777 | 0.7556 | 0.0013 | 0.0359 |

TABLE 38
Activity Data

| | Slope | | | |
|---|---|---|---|---|
| Sample | (A/min) | U (umol/min) | U Avg | U SD |
| Blank | 0.0004 | 0.0842 | 0.08 | NA |
| Control | 0.0004 | 0.0884 | 0.09 | 0.01 |
| | 0.0004 | 0.0937 | | |
| | 0.0005 | 0.0992 | | |
| Lipase (100 mg/ml | 0.0095 | 2.0796 | 2.12 | 0.15 |
| wet) | 0.0105 | 2.2884 | | |
| | 0.0091 | 1.9857 | | |

TABLE 39
Absorbance vs. Time Slope

| Sample | U (μmol/min) |
|---|---|
| Blank | 0.08 + 0.00 |
| Control | 0.09 + 0.01 |
| Lipase | 2.12 + 0.15 |

### Example 28

This Example demonstrates lipase activity in a Glidden alkyd/oil solvent-borne coating. The materials used are shown in the table below.

TABLE 40
Materials

200 mM TRIS Buffer (Sigma Product # T1503); brought to
pH = 7.1 with HCl
4-nitrophenyl acetate (Sigma Product # N8130); 14.5 mM solution in
isopropyl alcohol
Lipase from porcine pancreas (Sigma Product # L3126)
2 mL microtubes
Pipette
Pipette Tips
Plate Reader
96-well Plate

The assay procedure included: cutting appropriate coupon sizes; placing individual coupons into labeled 2 mL microtubes, with each of the coupon sizes are tested in triplicate; adding 750 ul 200 mM TRIS to each microtube; adding 600 ul ddH2O to each microtube; adding 150 ul 14.5 mM p-nitrophenyl acetate to each microtube; preparing control samples (no films) to have 750 ul 200 mM TRIS, 600 ul ddH2O, and 150 ul 14.5 mM p-nitrophenyl acetate; removing at each desired time point, 100ul and reading the absorbance at 405 nm in a 96-well plate; and plotting absorbance vs. time to calculate the initial rate slope.

TABLE 41A
Absorbance at 405 nm

| Time | Blank | | | 3 cm × 1 cm Control | | |
|---|---|---|---|---|---|---|
| 0 | 0.04430 | 0.04260 | 0.04420 | 0.04430 | 0.04260 | 0.04420 |
| 15 | 0.05450 | 0.04840 | 0.04940 | 0.05290 | 0.05300 | 0.04810 |
| 30 | 0.05520 | 0.05400 | 0.05520 | 0.05530 | 0.05720 | 0.05160 |
| 60 | 0.06710 | 0.06520 | 0.06730 | 0.06180 | 0.06230 | 0.05970 |
| 120 | 0.07800 | 0.07690 | 0.07810 | 0.06770 | 0.06820 | 0.07120 |
| Slope | 0.00027 | 0.00029 | 0.00029 | 0.00018 | 0.00019 | 0.00023 |

TABLE 41B
Absorbance at 405 nm

| Time | 3 cm × 1 cm Lipase 200 g/gal | | | 3 cm × 1 cm Lipase 100 g/gal | | |
|---|---|---|---|---|---|---|
| 0 | 0.04430 | 0.04260 | 0.04420 | 0.04430 | 0.04260 | 0.04420 |
| 15 | 0.07050 | 0.11020 | 0.06940 | 0.05300 | 0.05260 | 0.05300 |
| 30 | 0.07970 | 0.11690 | 0.07850 | 0.06280 | 0.06780 | 0.06270 |
| 60 | 0.10290 | 0.12410 | 0.09510 | 0.09460 | 0.08930 | 0.08780 |
| 120 | 0.13500 | 0.15060 | 0.12870 | 0.10620 | 0.12110 | 0.11940 |
| Slope | 0.00071 | 0.00069 | 0.00065 | 0.00054 | 0.00066 | 0.00064 |

TABLE 42A
Absorbance Averages

| | Absorbance Average | | | |
|---|---|---|---|---|
| Time | Blank | Control | 200 g/gal | 100 g/gal |
| 0 | 0.04370 | 0.04370 | 0.04370 | 0.04370 |
| 15 | 0.05077 | 0.05133 | 0.08337 | 0.05287 |
| 30 | 0.05480 | 0.05470 | 0.09170 | 0.06443 |
| 60 | 0.06653 | 0.06127 | 0.10737 | 0.09057 |
| 120 | 0.07767 | 0.06903 | 0.13810 | 0.11557 |

TABLE 42B
Absorbance Average's Standard Deviations

Absorbance Deviation

| Time | Blank | Control | 200 g/gal | 100 g/gal |
|---|---|---|---|---|
| 0 | 0.000954 | 0.000954 | 0.000954 | 0.000954 |
| 15 | 0.003272 | 0.002801 | 0.023245 | 0.000231 |
| 30 | 0.000693 | 0.002848 | 0.021832 | 0.002916 |
| 60 | 0.001159 | 0.00138 | 0.015007 | 0.003573 |
| 120 | 0.000666 | 0.001893 | 0.011274 | 0.008156 |

TABLE 43
Absorbance vs. Time Slope

| | Slope | | | |
|---|---|---|---|---|
| Sample | (A/min) | U (umol/min) | U Average | U Deviation |
| Blank | 0.000267 | 0.0584 | 0.06 | 0.00 |
| | 0.000285 | 0.0624 | | |
| | 0.000285 | 0.0625 | | |
| Control 3 cm² | 0.000177 | 0.0388 | 0.04 | 0.01 |
| | 0.000187 | 0.0410 | | |
| | 0.000226 | 0.0494 | | |
| 200 g/gal 3 cm² | 0.000707 | 0.1548 | 0.15 | 0.01 |
| | 0.000687 | 0.1503 | | |
| | 0.000648 | 0.1418 | | |
| 100 g/gal 3 cm² | 0.000540 | 0.1182 | 0.13 | 0.01 |
| | 0.000657 | 0.1437 | | |
| | 0.000639 | 0.1399 | | |

### Example 29

This Example demonstrates the effectiveness of lysozyme in lysing the bacterium Micrococcus lysodeikticus, M. lysodeikticus was used as a lysozyme substrate in a liquid suspension in the assay. The assay measured the rate of decrease in the absorbance as a relative measure of the amount/availability/activity of a lysozyme present in a material. As cell lysis occurs, the turbidity of a cell suspension decreased, and therefore, the absorbance of a cell suspension decreased. Materials and reagents that were used are shown in the table below.

TABLE 44
Materials and Reagents

2 M sodium phosphate buffer (NaH₂PO₄), pH 6.4, or Tris-HCL Buffer,
pH 7.0
Micrococcus lysodeikticus cell (Worthington Biochemicals, #8736)
Lysozyme (chicken egg white) (Sigma Product #L 6876,
CAS 12650-88-3)
96-well plate
Thermo Multiskan Ascent Plate Reader
Pipettes and Pipetteman
Microtubes

The reagents that were prepared included a M. lysodeikticus cell suspension comprising 9 mg M. lysodeikticus in 25 mL sodium phosphate buffer, and a lysozyme solution comprising a 5 mg/mL stock solution.

The assay procedure included diluting the lysozyme stock solution with buffer to create the following samples: 5 mg/mL (undiluted); 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL; and 0.00005 mg/mL. Control samples included: 3 replicates of 200 μL M. lysodeikticus cell suspension and 3 replicates of 200 μL buffer that were pipetted into 6 wells total in a 96-well microplate. A 194 μL Micrococcus cell suspension was pipetted into 3 rows of 12 wells each. 6 μL of each lysozyme concentration assayed was then added to the M. lysodeikticus cell suspension using a multi-pipette and mixed. The plate was immediately placed into the Thermo Multiskan Ascent Plate Reader; each well was read every 10 seconds for 30 minutes to determine the absorbance at 450 nm.

TABLE 45
Lysis of M. lysodeikticus (Ml) over a concentration range of lysozyme

| Lysozyme | | | | | Ml lysed |
|---|---|---|---|---|---|
| (mg × 10⁻³) | Abs | Time (sec) | dAbs | dAbs/sec | (mg × 10⁻⁶)/sec |
| 0.01 | 0.37 | 1800 | 0.015 | 8.33 × 10⁻⁶ | 1.6 |
| 0.02 | 0.35 | 1800 | 0.035 | 1.94 × 10⁻⁵ | 3.6 |
| 0.1 | 0.31 | 1800 | 0.075 | 4.17 × 10⁻⁵ | 7.8 |
| 0.2 | 0.22 | 1800 | 0.165 | 9.17 × 10⁻⁵ | 17.1 |
| 1 | 0.275 | 300 | 0.11 | 3.67 × 10⁻⁴ | 68.6 |
| 2 | 0.13 | 520 | 0.255 | 4.9 × 10⁻⁴ | 91.7 |
| 10 | 0.26 | 2 | 0.125 | 6.25 × 10⁻² | 11688.3 |
| 20 | 0.23 | 2 | 0.155 | 7.75 × 10⁻² | 14493.5 |
| 100 | 0.165 | 2 | 0.22 | 1.1 × 10⁻¹ | 20571.4 |

TABLE 46
Summary of Activity

| | |
|---|---|
| Abs | 0.38 |
| [Ml] | 0.36 mg/ml |
| Vol | 0.2 ml |
| | 0.187 dmg/dOD |
| Rate | 0.047 dmg Ml/sec/mg |
| | lysozyme |

The results for the lysozyme assay under the conditions as described: 1 mg of lysozyme was able to lyse 0.047 mg of M. lysodeikticus per sec. The lysozyme was effective in lysing M. lysodeikticus cells, and these results were consistent under both conditions evaluated (Tris vs NaH₂PO₄)

### Example 30

This Example demonstrates the ability of a lysozyme to survive the incorporation process into a coating, demonstrates lysozyme hydrolytic activity in a coating environment, and demonstrates the ability of lysozyme to survive in can conditions for 48 hours. A Sherwin-Williams Acrylic Latex paint was used. Materials, reagents and equipment used are shown in the tables below.

TABLE 47
Materials and Reagents

0.1 M potassium phosphate buffer, pH 6.4
Micrococcus lysodeikticus (Worthington Biochemicals, #8736)
Sherwin-Williams Acrylic Latex paint
Lysozyme (chicken egg white) (Sigma Product #L 6876,
CAS 12650-88-3)
15 mL plastic test tubes

TABLE 48
Equipment

Paint spreader (1-8 mil)
Polypropylene blocks
Lightnin Labmaster Mixer
Rotator shaker
Pipettes and Pipetteman
Klett-Sumerson Colorimeter (Filter D35: 540 nm)

The reagents prepared included a Micrococcus cell suspension comprising 9 mg M. lysodeikticus in 25 mL sodium phosphate buffer, and a lysozyme solution comprising a 5 mg/mL stock solution. The paint formulations used are shown in the table below.

TABLE 49
Paint Preparation

Sherwin-Williams Acrylic Latex Control (no additive)
Sherwin-Williams Acrylic Latex with 1 mg/mL lysozyme

The paint was mixed with a glass stirring rod and a paint mixer. Each film was immediately drawn onto polypropylene surfaces with a thickness of 8 mil. Cure time for the Sherwin-Williams was 72 hrs. To demonstrate in can durability, the Sherwin-Williams Acrylic Latex comprising lysozyme wet paint was sealed and shelf stored at ambient temperature. After 48 hrs in can, films were drawn onto polypropylene surfaces with a thickness of 8 mils and were allowed to cure 72 hrs prior to assay. Coupons were generated as free films from the polypropylene surface. Films were generated in three sizes: 2 cm²: 1 cm by 2 cm; 4 cm²: 1 cm by 4 cm; or 6 cm²: 1 cm by 6 cm.

For qualitative assessment, individual films were placed into labeled 15 mL tubes. Films of each size (2, 4 and 6 cm²) were evaluated in triplicate. In addition to a control paint with no additive, two other controls were utilized, a positive control and a negative control. The positive control comprised: lysozyme in buffer added to each of three 15 mL tubes in concentrations approximating the amount of lysozyme in the films (i.e., 40 μg, 80 μg, and 120 μg). Each amount was assayed in triplicate. The negative control comprised: 5 mL of 0.36 mg/mL M. lysodeikticus cell suspension pipetted into a single 15 mL tube. 5 mL 0.36 mg/mL Micrococcus lysodeikticus cell suspension was added to all reaction tubes to begin the reaction. The tubes were placed on a rocker at ambient conditions for approximately 22 hours. Where possible, the films were removed from the suspension and determine opacity using the Klett-Summerson Colorimeter (turbidity unit: Klett Unit or KU).

Particulate matter in the samples interfered with quantitation; photographs of each set of 2 cm² paint films and controls following 22 hour contact to M. lysodeikticus cell suspension were taken, and observations recorded in the Tables below.

TABLE 50
Qualitative Observations (visual assessments)

| Sample¹ | Lysozyme (μg) | Film Size (cm²) | Clarity |
|---|---|---|---|
| Suspension/Solution | | | |
| Controls | | | |
| M. lysodeikticus | — | — | Translucent |
| Lysozyme | 40 | — | Transparent² |
| | 80 | — | Transparent |
| | 120 | — | Transparent |
| Control Films | | | |
| S-W | | 2, 4, 6 | Translucent |
| Films Comprising | | | |
| Lysozyme | | | |
| S-W | | 2, 4, 6 | Transparent |

¹Each evaluation was performed in triplicate
²Thinned in opacity, with some suspended particulate matter

The strips comprising lysozyme of all three sizes of coupons cleared the M. lysodeikticus suspension, indicating that the lysozyme maintains activity in the coating environment. Cleared suspensions (lysozyme comprising coupons and controls) comprised large particles which interfere with the quantitation of the cleared suspensions. The particulate matter was less detectable in the 2 cm² set comprising lysozyme, so this size coupon was used for the quantitative demonstrations.

TABLE 51
Quantiative Assessment of Lysozyme In-Film Activity (2 cm² film, 4 hr
time point, 3 independent assays, each performed in triplicate.)

| | | Replicate 1 | | Replicate 2 | | Replicate 3 | |
|---|---|---|---|---|---|---|---|
| | In can | | Cell | | Cell | | Cell |
| Formulation | (hrs) | KU | lysis | KU | lysis | KU | lysis |
| Suspension Controls | | | | | | | |
| M. lysodeikticus | | 81.5 | 0.0% | 101 | 0% | | |
| Lysozyme | | 17 | | 27 | | | |
| S-W Acrylic Latex | | | | | | | |
| Control Films | — | 75 | 18% | 74 | 19% | 71 | 22% |
| | — | 79 | 13% | 82 | 10% | 76 | 17% |
| | — | 83 | 9% | 81 | 11% | 73 | 20% |
| Films Comprising | — | 8 | 91% | 20 | 78% | 11 | 88% |
| Lysozyme | — | 13 | 86% | 11 | 88% | 15 | 84% |
| | — | 13 | 86% | 5 | 95% | 0 | 100% |
| Control Films | 48 hrs | 82 | 10% | 65 | 29% | 68 | 25% |
| Films Comprising | 48 hrs | 36 | 61% | 26 | 72% | 37 | 59% |
| Lysozyme | | | | | | | |

KU = Klett Units, measure of turbidity at 540 nm.

A lysozyme in Sherwin-Williams Acrylic Latex was able to lyse about 88% of the M. lysodeikticus culture over 4 hours, relative to the control which exhibited about a 15% drop in opacity. After in-can shelving for 48 hrs (i.e., the lysozyme was mixed into the Sherwin-Williams Acrylic Latex, capped and shelved for 48 hrs prior to drawing down the films), the lysozyme remained active, lysing about 64% of the M. lysodeikticus culture relative to the about 21% lysis exhibited by the control panels.

### Example 31

This Example demonstrates the retention of lysozyme vs. loss due to leaching in a paint film in a saturated condition at 1, 2 and 24 hours after submersion. Materials, reagents and equipment used are shown in the tables below.

TABLE 52
Materials and Reagents

0.1 M potassium phosphate buffer, pH 6.4
Micrococcus lysodeikticus (Worthington Biochemicals, #8736)
Lysozyme (chicken egg white) (Sigma Product #L 6876, CAS
12650-88-3)
Sherwin-Williams Acrylic Latex paint
15 mL plastic test tubes

TABLE 53
Equipment

Paint spreader (1-8 mil)
Polypropylene blocks
Lightnin Labmaster Mixer
Rotator shaker
Pipetter and tips
Klett-Sumerson Colorimeter (Filter D35: 540 nm)

The reagents prepared included a Micrococcus cell suspension comprising 9 mg M. lysodeikticus in 25 mL sodium phosphate buffer, and a lysozyme solution comprising a 5 mg/mL stock solution.

The paint formulations that were prepared included a Sherwin-Williams Acrylic Latex Control (no additive), and a Sherwin-Williams Acrylic Latex comprising 1 mg/mL lysozyme. Each paint was mixed with a glass stirring rod and a paint mixer. Each film was immediately drawn onto polypropylene surfaces with a thickness of 8 mil. Cure time was 120 hrs. The Sherwin-Williams Acrylic Latex comprising a lysozyme wet paint was sealed and shelf stored at ambient temperature. After 48 hrs in can storage, films were drawn onto polypropylene surfaces with a thickness of 8 mils and were allowed to cure 72 hrs prior to assay. Materials for assay were generated from the polypropylene surface as a 2 cm² (1×2 cm) free film.

The assay procedure included placing individual films into labeled 15 mL tubes. 24 hours prior to addition of Micrococcus lysodeikticus cell suspension, 5 mL KPO₄ buffer was added to the 24-hour control and coupon comprising a lysozyme tube, as well as one tube comprising 41 μg lysozyme solution (positive control) and one tube comprising 5 mL of the M. lysodeikticus cell suspension (negative control). These tubes were placed on the shaker for 24 hrs.

2 hours prior to addition of M. lysodeikticus, 5 mL potassium phosphate buffer was added to the 2-hour control and lysozyme tubes each comprising a coupon, as well as one tube comprising 41 μg lysozyme solution (positive control) and one tube comprising 5 mL of the M. lysodeikticus cell suspension (negative control). These tubes were placed on the shaker for 2 hrs.

1 hour prior to addition of M. lysodeikticus cell suspension, 5 mL potassium phosphate buffer was added to 1-hour control and coupon comprising a lysozyme tubes, as well as one tube comprising 41 μg lysozyme solution (positive control) and one tube comprising 5 mL of the M. lysodeikticus cell suspension (negative control). These tubes were placed on the shaker for one hour.

The paint coupons were then transferred from each tube to a second reaction tube. 5 mL of the M. lysodeikticus cell suspension was added to both film and KPO₄ buffer incubation buffer. The tubes were placed on the rotating shaker horizontally and shaken for approximately 4 hours, at which time each tube was measured in a Klett-Summerson Photoelectric Colorimeter to determine opacity.

TABLE 54
Assessment of lysis and enzyme leaching (free film) after 1, 2 and 24 hr,
relative to the internal control (i.e., the no lysozyme films).

| | | Replicate 1 | | Replicate 2 | | Replicate 3 | | Average | |
|---|---|---|---|---|---|---|---|---|---|
| | | | Cell | | Cell | | Cell | | Cell |
| | Time | | lysis | | lysis | | lysis | | Lysis |
| Formulation | (hrs) | KU | (dKU) | KU | (dKU) | KU | (dKU) | KU | (dKU) |
| KPO₄ Buffer | | | | | | | | | |
| Control | 1 hr | 110 | 0% | 90 | 0% | 104 | 0% | 101 | 0% |
| Lysozyme | 1 hr | 62 | 39% | 42 | 59% | 52 | 49% | 52 | 49% |
| Control | 2 hr | 92 | 0% | 102 | 0% | 106 | 0% | 100 | 0% |
| Lysozyme | 2 hr | 74 | 26% | 65 | 35% | 65 | 35% | 68 | 32% |
| Control | 24 hr | 95 | 0% | 95 | 0% | 92 | 0% | 94 | 0% |
| Lysozyme | 24 hr | 80 | 15% | 62 | 34% | 55 | 41% | 66 | 30% |
| Film | | | | | | | | | |
| Control | 1 hr | 64 | 0% | 54 | 0% | 38 | 0% | 52 | 0% |
| Lysozyme | 1 hr | 3 | 94% | 40 | 23% | 4 | 92% | 16 | 81% |
| Control | 2 hr | 63 | 0% | 73 | 0% | 72 | 0% | 69 | 0% |
| Lysozyme | 2 hr | 10 | 86% | 23 | 67% | 45 | 35% | 26 | 54% |
| Control | 24 hr | 65 | 0% | 65 | 0% | 68 | 0% | 66 | 0% |
| Lysozyme | 24 hr | 30 | 55% | 52 | 21% | 52 | 21% | 45 | 32% |

KU = Klett Unit, measure of turbidity at 540 nm

At the three time points assayed, lysozyme leached out of films that comprised a lysozyme. The ability of the films comprising a lysozyme to lyse M. lysodeikticus was inversely related to the time the coupon was submerged. Over the first 2 hrs the films lost approximately 21%±3% of the lytic activity per hour. This loss decreased substantially over the following 22 hrs, with the loss slowing to approximately 3% per hour. After 24 hours of liquid submersion, approximately one-third of the activity of a coupon comprising a lysozyme was retained. Though reduction of activity due to leaching may continue, activity may also be permanently retained in the films. The total percentage lysis by coupon and buffer pairs decreased with increasing leaching time.

### Example 32

This Example demonstrates the surface efficacy of paint films comprising a lysozyme in actively lyse M. lysodeikticus in a minimally hydrated environment. Materials, reagents and equipment used are shown in the tables below.

TABLE 55
Materials and Reagents

0.1 M potassium phosphate buffer, pH 6.4
Micrococcus lysodeikticus (Worthington Biochemicals, #8736)
Lysozyme (chicken egg white) (Sigma Product #L 6876, CAS
12650-88-3)
Sherwin-Williams Acrylic Latex paint
15 mL plastic test tubes

TABLE 56
Equipment

Paint spreader (1-8 mil)
Polypropylene blocks
Lightnin Labmaster Mixer
Rotator shaker
Pipetter and tips
Klett-Sumerson Colorimeter (Filter D35: 540 nm)

The reagents prepared included a Micrococcus cell suspension comprising 9 mg Micrococcus lysodeikticus in 25 mL sodium phosphate buffer, and a lysozyme solution comprising a 5 mg/mL stock solution.

The paint formulations prepared for the assay included a Sherwin-Williams Acrylic Latex Control (no additive), and a Sherwin-Williams Acrylic Latex with 1 mg/mL lysozyme. Each paint was mixed with a glass stirring rod and a paint mixer. Each film was immediately drawn onto polypropylene surfaces with a thickness of 8 mil. Cure time was 72 hrs. Assay materials were generated from the polypropylene surface as a 2 cm² (1×2 cm) free film.

The assay procedure included placing individual coupons into separate Petri dishes. Each set of control coupons and coupons comprising a lysozyme was assayed in triplicate. Two controls were set up for this experiment: a M. lysodeikticus suspension control comprising 90 μL 20 mg/mL M. lysodeikticus cell suspension that was pipetted into a petri dish; and a 1 mg/mL lysozyme control comprising 40.64 μL 1 mg/mL lysozyme solution (an amount approximately equal to the amount of lysozyme in the 2 cm² coupon comprising a lysozyme) that was pipetted into a petri dish. M. lysodeikticus cell suspension was distributed onto the surface of each individual coupon in a minimal volume (90 μL). Petri dishes were kept on a flat surface. After 4 hours, KPO₄ buffer was added to all samples to recover the unlysed portion of the M. lysodeikticus cell suspension. The suspension was removed from each dish with a pipette and placed into individual test tubes. Each suspension was read in the Klett-Summerson Photoelectric Colorimeter, using potassium phosphate buffer as a control.

TABLE 57
Surface Efficacy of Films comprising lysozyme in a low hydration
environment.

| | Replicate 1 | | Replicate 2 | | Replicate 3 | | Average | |
|---|---|---|---|---|---|---|---|---|
| | | Cell | | Cell | | Cell | | Cell |
| Formulation | KU | lysis | KU | lysis | KU | lysis | KU | Lysis |
| Suspension/ | | | | | | | | |
| Solution | | | | | | | | |
| Controls | | | | | | | | |
| M. | 80 | | | | | | | |
| lysodeikticus | | | | | | | | |
| Lysozyme | 10 | | | | | | | |
| S-W Acrylic | | | | | | | | |
| Latex | | | | | | | | |
| Control Films | 75 | 6% | 70 | 13% | 78 | 3% | 74 | 7% |
| Lysozyme | 35 | 56% | 19 | 76% | 31 | 61% | 28 | 65% |
| Films | | | | | | | | |

KU = Klett units, measure of turbidity at 540 nm.

The paint comprising a lysozyme contacted with 0.18 mg of a M. lysodeikticus suspension for 4 hours lysed 65%±10% of the Micrococcus cells, compared to only 7%±5% of cells lysed by the paint controls. This demonstrated that lysozyme can function in the low water (i.e., a minimally hydrated) environment of a coating. It is contemplated that a biological assay including a spray application of an assay organism would also demonstrate biostatic and/or biocidal activity.

### Example 33

This Example demonstrates the ability of a chymotrypsin to survive the incorporation process into a coating and demonstrates chymotrypsin activity in a coating environment. A chymotrypsin free film assay was used for determining the activity of chymotrypsin, as measured by ester hydrolysis (esterase) activity of a p-nitrophenyl acetate substrate, in free-films using a plate reader. A functioning vent hood was used for the assay when appropriate for material handling. A Sherwin-Williams Acrylic Latex paint was used. Equipment and reagents that were used are shown in the tables below.

TABLE 58
Equipment

Plate Reader
2 ml microtubes

TABLE 59
Reagents

α-Chymotrypsin from bovine pancreas, Type II (Sigma Cat# C4129)
4-Nitrophenyl acetate, MW 181.15 (Sigma Cat# N8130)
Trizma base (Sigma Cat# T1503)

Sample preparation included: 14.5 mM p-nitrophenyl acetate (66 mg/25 ml) in isopropyl alcohol, and 200 mM TRIS; pH 7.1 (adjust to pH 7.1 with HCl).

The paint formulations that were prepared included a Sherwin-Williams Acrylic Latex control (no additive), and a Sherwin-Williams Acrylic Latex comprising 200 mg/mL α-Chymotrypsin. Each paint was mixed with a glass stirring rod and a paint mixer. Each film was immediately drawn onto polypropylene surfaces with a thickness of 8 mil. Cure time was 24 days. Materials for assay were generated from the polypropylene surface as 1 cm², 2 cm² and 3 cm² free films.

The plate reader assay comprised: cutting free films into appropriate size pieces; adding 600 μL ddH2O into a 2 ml microtube; then adding 750 μL 200 mM TRIS to each microtube; adding 150 μL of 14.5 mM p-nitrophenyl acetate to each tube; and taking the 0 time sample, then adding the free film to the tube (control sample is free film with no chymotrypsin).

The analysis included: taking out 100 μl and reading the absorbance at 405 nm, at the appropriate time points; and determining the initial rate slope by plotting absorbance vs. time to calculate chymotrypsin activity.

TABLE 60A
Absorbance at 405 nm Chymotrypsin in Sherwin-Williams Acrylic
Latex

| Time | Blank | | | 3 cm × 1 cm Control | | |
|---|---|---|---|---|---|---|
| 0 | 0.0480 | 0.0429 | 0.0446 | 0.0480 | 0.0429 | 0.0446 |
| 15 | 0.0482 | 0.0489 | 0.0479 | 0.0518 | 0.0541 | 0.0541 |
| 30 | 0.0571 | 0.0558 | 0.0555 | 0.0596 | 0.0612 | 0.0609 |
| 45 | 0.0608 | 0.0617 | 0.0617 | 0.0679 | 0.0709 | 0.0690 |
| 60 | 0.0683 | 0.0690 | 0.0679 | 0.0773 | 0.0826 | 0.0781 |
| Slope | 0.0004 | 0.0004 | 0.0004 | 0.0005 | 0.0006 | 0.0005 |

TABLE 60B
Absorbance at 405 nm Chymotrypsin in Sherwin-Williams Acrylic
Latex

| Time | 3 cm × 1 cm Enzyme | | | 2 cm × 1 cm Enzyme | | |
|---|---|---|---|---|---|---|
| 0 | 0.0480 | 0.0429 | 0.0446 | 0.0480 | 0.0429 | 0.0446 |
| 15 | 0.2364 | 0.2356 | 0.2347 | 0.1690 | 0.1801 | 0.1749 |
| 30 | 0.4504 | 0.4375 | 0.4208 | 0.3040 | 0.3149 | 0.3172 |
| 45 | 0.6395 | 0.6267 | 0.6441 | 0.4348 | 0.4579 | 0.4474 |
| 60 | 0.8358 | 0.7957 | 0.7970 | 0.5682 | 0.5942 | 0.5930 |
| Slope | 0.0132 | 0.0126 | 0.0128 | 0.0087 | 0.0092 | 0.0091 |

TABLE 60C
Absorbance at 405 nm
Chymotrypsin in Sherwin-Williams Acrylic Latex

| Time | 1 cm × 1 cm Enzyme | | |
|---|---|---|---|
| 0 | 0.0480 | 0.0429 | 0.0446 |
| 15 | 0.1156 | 0.1155 | 0.1164 |
| 30 | 0.1886 | 0.1932 | 0.1872 |
| 45 | 0.2688 | 0.2745 | 0.2684 |
| 60 | 0.3427 | 0.3479 | 0.3578 |
| Slope | 0.0050 | 0.0051 | 0.0052 |

TABLE 61A
Absorbance Averages Chymotrypsin in Sherwin-Williams Acrylic Latex

| | Absorbance Average | | | | |
|---|---|---|---|---|---|
| | | | Chymotrypsin | Chymotrypsin | |
| Time | Blank | Control 3 cm² | 3 cm² | 2 cm² | Chymotrypsin 1 cm² |
| 0 | 0.0452 | 0.0452 | 0.0452 | 0.0452 | 0.0452 |
| 15 | 0.0483 | 0.0533 | 0.2356 | 0.1747 | 0.1158 |
| 30 | 0.0561 | 0.0606 | 0.4362 | 0.3120 | 0.1897 |
| 45 | 0.0614 | 0.0693 | 0.6368 | 0.4467 | 0.2706 |
| 60 | 0.0684 | 0.0793 | 0.8095 | 0.5851 | 0.3495 |

TABLE 61B
Absorbance Averages Standard Deviations Chymotrypsin in Sherwin-
Williams Acrylic Latex

| | Absorbance Standard Deviation | | | | |
|---|---|---|---|---|---|
| Time | Blank | Control 3 cm² | Chymotrypsin 3 cm² | Chymotrypsin 2 cm² | Chymotrypsin 1 cm² |
| 0 | 0.0026 | 0.0026 | 0.0026 | 0.0026 | 0.0026 |
| 15 | 0.0005 | 0.0013 | 0.0009 | 0.0056 | 0.0005 |
| 30 | 0.0009 | 0.0009 | 0.0148 | 0.0071 | 0.0031 |
| 45 | 0.0005 | 0.0015 | 0.0090 | 0.0116 | 0.0034 |
| 60 | 0.0006 | 0.0029 | 0.0228 | 0.0147 | 0.0077 |

TABLE 62
Absorbance vs. Time Slope

| | Slope | | | |
|---|---|---|---|---|
| Sample | (A/min) | U (umol/min) | U Average | U Deviation |
| Blank | 0.0004 | 0.0776 | 0.09 | 0.01 |
| | 0.0004 | 0.0949 | | |
| | 0.0004 | 0.0881 | | |
| Control 3 cm² | 0.0005 | 0.1090 | 0.12 | 0.02 |
| | 0.0006 | 0.1404 | | |
| | 0.0005 | 0.1195 | | |
| Chymotrypsin 3 cm² | 0.0132 | 2.8876 | 2.82 | 0.06 |
| | 0.0126 | 2.7679 | | |
| | 0.0128 | 2.7935 | | |
| Chymotrypsin 2 cm² | 0.0087 | 1.9062 | 1.97 | 0.06 |
| | 0.0092 | 2.0145 | | |
| | 0.0091 | 1.9983 | | |
| Chymotrypsin 1 cm² | 0.0050 | 1.0837 | 1.11 | 0.03 |
| | 0.0051 | 1.1222 | | |
| | 0.0052 | 1.1359 | | |

A chymotrypsin in Sherwin-Williams Acrylic Latex was able to hydrolyze the model substrate at rate 20× faster than the control. The test coupons demonstrate a dose response which corresponds to a hydrolytic capacity of 0.86 umol/min/cm², as formulated in this demonstration.

Quality control included reading and become familiar with the operating instructions for equipment used in the analysis. Operating instructions and preventive maintenance records were placed near the relevant equipment, and kept in a labeled central binder in the work area. Working solutions which are out of date or prepared incorrectly were disposed of and not used.

Safety procedures and precautions included wearing a full length laboratory coat; and not eating, drinking, smoking, use of tobacco products or application of cosmetics near the procedure. Consumables and disposable items that come in contact with or are used in conjunction with samples disposal were in the proper hazard containers. This includes, but is not limited to, pipette tips, bench-top absorbent paper, diapers, kimwipes, test tubes, etc. Biohazard containers were considered full when their contents reach three-quarters of the way to the top of the bag or box. Bench-top biohazard bags were placed into a large biohazard burn box when full. Biohazard containers were not filled to overflowing. Biohazard bags were disposed of by closing with autoclave tape, and autoclaving immediately. Spills and spatters were immediately cleaned from durable surfaces by applying 70% ethanol (for bacteriological spills) to the spill, followed by wiping or blotting. All equipment used in sample analyses were wiped down on a daily basis or whenever tests were performed. Absorbent pads were placed under samples when useful. Hands were washed with antibacterial soap before exiting the room, when a test was finished, and before the end of the day. The Material Safety Data Sheet (“MSDS”) applicable to each chemical was read. MSDS documents have been prominently posted in the laboratory. During a fire alarm during laboratory operations, evacuation procedures were followed. Nitrile protective gloves were worn whenever handling organophosphates. All organophosphate waste was disposed of properly.

### Example 34

This Example demonstrates the ability of a cellulase to survive the incorporation process into a coating and demonstrates cellulase activity in a coating environment. A Glidden Latex paint was used. A plate reader was used to assay a free-film comprising a cellulase for the enzyme's activity. Equipment and reagents that were used are shown in the table below.

TABLE 63
Equipment and Reagents

Equipment
Plate Reader
Reagents
Sodium Acetate (Sigma Cat# S8625)
4-Nitrophenyl β-D-cellobioside (Sigma Cat# N5759)
Cellulase (TCI Cat# C0057)
Sodium Hydroxide

Sample preparation included: 14.5 mM 4-Nitrophenyl β-D-cellobioside in ddH2O; 50 mM sodium acetate buffer; pH 5.0 (adjust to pH 5.0 with HCl); and 2 N NaOH in ddH2O.

The plate reader assay comprised: placing free films into 2 ml microtubes; add 1.2 ml 50 mM sodium acetate buffer, 0.15 ml 14.5 mM 4-Nitrophenyl β-D-cellobioside and 0.15 ml ddH2O, in the 2 ml microtube; placing tubes on rocker; taking out 100 μl from the tubes into a 96-well plate at desired time points; adding 200 μl of 2 N NaOH and reading the absorbance at 405 nm; and determining the initial rate slope by plotting absorbance vs. time to calculate cellulase activity.

The paint formulations that were prepared included a Sherwin-Williams Acrylic Latex control (no additive), and a Sherwin-Williams Acrylic Latex comprising 100 g/gal, 200 g/gal and 300 g/gal cellulase. Each paint was mixed with a glass stirring rod and a paint mixer. Each film was immediately drawn onto polypropylene surfaces with a thickness of 8 mil. Cure time was 24 hrs. Materials for assay were generated from the polypropylene surface as a 3 cm² free film.

TABLE 64A
Glidden Latex Cellulase Free Films - Dose Response - pNP Absorbance at
405 nm

| Time (min) | Blank | Control | | | 100 g/gal | | |
|---|---|---|---|---|---|---|---|
| 0 | 0.0600 | 0.0600 | 0.0600 | 0.0600 | 0.0600 | 0.0600 | 0.0600 |
| 30 | 0.0496 | 0.0588 | 0.0488 | 0.0476 | 0.0744 | 0.0753 | 0.0716 |
| 60 | 0.0496 | 0.0605 | 0.0505 | 0.0532 | 0.0975 | 0.1158 | 0.1007 |
| 120 | 0.0507 | 0.0519 | 0.0522 | 0.0514 | 0.1691 | 0.1823 | 0.1672 |
| 180 | 0.0550 | 0.0643 | 0.0583 | 0.0511 | 0.2351 | 0.2312 | 0.2073 |
| 240 | 0.0512 | 0.0614 | 0.0518 | 0.0548 | 0.2876 | 0.2919 | 0.2720 |
| 300 | 0.0491 | 0.0574 | 0.0601 | 0.0575 | 0.3187 | 0.3123 | 0.3083 |
| 360 | 0.0528 | 0.0680 | 0.0540 | 0.0655 | 0.3322 | 0.3215 | 0.3309 |
| Slope (A/min) | −0.0001 | −0.0001 | 0.0000 | 0.0000 | 0.0009 | 0.0011 | 0.0009 |

TABLE 64B
Glidden Latex Cellulase Free Films - Dose Response - pNP
Absorbance at 405 nm

| Time (min) | 200 g/gal | | | 300 g/gal | | |
|---|---|---|---|---|---|---|
| 0 | 0.0600 | 0.0600 | 0.0600 | 0.0600 | 0.0600 | 0.0600 |
| 30 | 0.0986 | 0.0866 | 0.0927 | 0.1207 | 0.1170 | 0.1146 |
| 60 | 0.1387 | 0.1341 | 0.1432 | 0.1637 | 0.1711 | 0.1670 |
| 120 | 0.2285 | 0.2219 | 0.2364 | 0.2864 | 0.2685 | 0.2965 |
| 180 | 0.2891 | 0.2740 | 0.3071 | 0.3304 | 0.3262 | 0.3833 |
| 240 | 0.3174 | 0.3281 | 0.3270 | 0.3543 | 0.3638 | 0.4118 |
| 300 | 0.3449 | 0.3467 | 0.3511 | 0.3759 | 0.3891 | 0.4051 |
| 360 | 0.3714 | 0.3588 | 0.3632 | 0.3808 | 0.3964 | 0.3651 |
| Slope (A/min) | 0.0014 | 0.0014 | 0.0015 | 0.0019 | 0.0017 | 0.0020 |

TABLE 65A
Glidden Latex Cellulase Free Films - Dose Response -
pNP Absorbance at 405 nm Averages

| | Average | | | | |
|---|---|---|---|---|---|
| Time (min) | Blank | Control | 100 g/gal | 200 g/gal | 300 g/gal |
| 0 | 0.0600 | 0.0600 | 0.0600 | 0.0600 | 0.0600 |
| 30 | 0.0496 | 0.0517 | 0.0738 | 0.0926 | 0.1189 |
| 60 | 0.0496 | 0.0547 | 0.1047 | 0.1387 | 0.1674 |
| 120 | 0.0507 | 0.0518 | 0.1729 | 0.2289 | 0.2775 |
| 180 | 0.0550 | 0.0579 | 0.2245 | 0.2901 | 0.3283 |
| 240 | 0.0512 | 0.0560 | 0.2838 | 0.3242 | 0.3591 |
| 300 | 0.0491 | 0.0583 | 0.3131 | 0.3476 | 0.3825 |
| 360 | 0.0528 | 0.0625 | 0.3282 | 0.3645 | 0.3886 |

TABLE 65B
Glidden Latex Cellulase Free Films -
Dose Response - pNP Absorbance at 405 nm
Averages' Deviations

| | |
|---|---|
| Time | Deviation |

| (min) | Control | 100 g/gal | 200 g/gal | 300 g/gal |
|---|---|---|---|---|
| 0 | 0.0000 | 0.0000 | 0.0000 | 0.0000 |
| 30 | 0.0061 | 0.0019 | 0.0060 | 0.0026 |
| 60 | 0.0052 | 0.0098 | 0.0046 | 0.0052 |
| 120 | 0.0004 | 0.0082 | 0.0073 | 0.0127 |
| 180 | 0.0066 | 0.0151 | 0.0166 | 0.0030 |
| 240 | 0.0049 | 0.0105 | 0.0059 | 0.0067 |
| 300 | 0.0015 | 0.0052 | 0.0032 | 0.0093 |
| 360 | 0.0075 | 0.0058 | 0.0064 | 0.0110 |

A cellulase in a Glidden Latex was able to hydrolyze the model substrate at a rate approximately 100× faster than the control. Quality control and safety procedures were as described in Example 33.

### Example 35

This Example demonstrates preparation of technical papers coated with a latex coating comprising an antimicrobial enzyme additive, an antimicrobial peptide additive, or a combination thereof. The additives may be embedded in the coating. The antimicrobial enzyme additive comprised lysozyme, and the antimicrobial peptide additive comprised PrateCoat® (Reactive Surfaces, Ltd.; also described in U.S. patent application Ser. Nos. 10/884,355; 11/368,086; and 11/865,514, each incorporated by reference). Materials that were used are shown in the tables below.

TABLE 66
Materials

30 mM Potassium Phosphate Buffer, was prepared by weighing out 416 mg of potassium phosphate into
2 × 50 mL conical tubes, and adding 50 mL of water to each tube.
Micrococcus lysodeikticus (Worthington Biochemicals, #8736), was prepared by weighing out 18 mg of
Micrococcus into a single 50 mL conical tube, adding KP0₄ buffer to 50 mLs, and mixing by inversion.
Lysozyme from chicken egg white (Sigma Product #L 6876; CAS no. 12650-88-3), was prepared by
weighing out 1 g, 0.5 g and 0.1 g lysozyme into 3 × 2 mL eppendorf tubes.
Dilute Acetic Acid Solution was prepared by measuring 1 mL of glacial acetic acid into 11 mLs of water
into a 15 mL conical tube, and adding 50 μl of the dilute acetic acid to 1 mL of water.
ProteCoat ® was used at 125 mg ProteCoat ® per g coating, dispensed as 250 mg ProteCoat ®, and
resuspended in 2 mL dilute acetic acid solution as appropriate.
5 × 15 mL conical tubes, glass stir rod
P1000 and P200 Pipetteman and Tips
5 × 15 mL conical tubes

Paint formulations comprising enzyme were prepared as follows: 1 g lysozyme per 100 g coating; 0.5 g lysozyme per 100 g coating; 0.1 9 lysozyme per 100 g coating; and a negative control (no additive). Paint formulations comprising a peptide additive were prepared as follows: 125 mg ProteCoat® per 1 g coating; 250 mg ProteCoat® per 1 g coating; 375 mg ProteCoat® per 1 g coating; or a negative control (no additive). Paint formulations comprising peptide and lysozyme were prepared as follows: 375 mg ProteCoat® per 1 g lysozyme (1 g) coating; 250 mg ProteCoat® per 1 g lysozyme (0.5 g) coating; 375 mg ProteCoat® per 1 g lysozyme (0.1 g) coating, and a negative Control (no additive). All paint formulations were mixed well. The paper was cut into quarters, coatings drawn onto paper surfaces with a spreader, and wet weight determined. The coated paper was dried at about 37.8° C. for approximately 10 min, and dry weight determined.

A single coating material and one paper stock was evaluated. The paper comprised celluosic fibers typically used in technical paper applications, and had an acrylic latex coating added to the fibers.

TABLE 67
Coating dry components added to paper

| Ingredient | % Dry Weight |
|---|---|
| Kaolin Clay (filler/pigment) | About 0.000000001% to about 90% |
| Titanium Dioxide (pigment) | About 0.000000001% to about 90% |
| Calcium Carbonate (filler/pigment) | About 0.000000001% to about 90% |
| Acrylic Latex (Binder) | About 0.000000001% to about 80% |

To prepare the antimicrobial paper (“AM-Paper”), the antimicrobial additives were formulated for each coating on percentage dry weight to standardize the coating for comparison. The antimicrobial additives are listed in the table below.

TABLE 68
Formulation details for antimicrobial papers

| | | | Final Dry | |
|---|---|---|---|---|
| | | Additive | Weight | |
| Antimicrobial | Designation | Formulation | (gsm) | Additive (%) |
| Control | | | 17.6 | None |
| | | | 21 | None |
| Enzymatic | A | Powder | 21.9 | 0.2% |
| | B | Powder | 19.4 | 1% |
| | C | Powder | 23.2 | 2% |
| | D | Suspension | 23 | 0.2% |
| | E | Suspension | 23 | 1% |
| | F | Suspension | 20.7 | 2% |
| ProteCoat ® | G | Suspension | 18.6 | 1% |
| | H | Powder | 23.9 | 2.5% |
| | I | Suspension | 20.6 | 0.5% |
| | J | Powder | 20.9 | 1.25% |
| | K | Powder | 20.9 | 0.25% |
| | L | Powder | 20.7 | 0.75% |
| Enzyme + Prote | | Powder | 22.5 | 2% + 0.5% |
| Coat ® | | Powder | 21.9 | 1% + 0.25% |

The antimicrobial additives were weighed out, added to pre-weighed coating suspensions and mixed by hand for 10 to 20 minutes. After mixing, the coating was applied by draw down, in which approximately 3-5 mLs of coating was applied along one 8.5″ edge of an 8.5″×11″ pre-weighed paper, and then spread evenly over the surface of the paper with a calibrated rod by drawing the rod down the full length of the paper. The coated paper was then placed into a 100° C. oven for 10 to 15 minutes to dry. After drying, the coated paper was weighed to determine the amount of coating on each sheet.

To conduct an assay to qualitatively assess antimicrobial activity, a paper strip of approximately 1 cm×5 cm was cut from the control and each antimicrobial paper. 5 mL of the M. lysodeikticus suspension was poured into each of 4×15 mL conical tubes. The prepared strip was dropped into the suspension, and mixed occasionally by inversion. Clearing changes were observed.

### Example 36

This Example demonstrates and provides a standard spectrophotometric assay procedure for lysozyme activity in a plate reader. Equipment and reagents that were used are shown in the table below.

TABLE 69
Equipment and Reagents

Equipment
Thermo Multiskan Ascent Plate Reader
96-well assay plates
Multi-channels and single-channel pipettes and tips
Reagents
Tris(hydroxymethyl)aminomethane hydrochloride (Tris-HCl): [Sigma, cat # T3253, Molecular Formula:
NH₂C(CH₂OH)₃•HCl, Molecular Weight: 157.60, CAS Number 1185-53-1, pKa (25° C.) 8.1]
Micrococcus lysodeikticus cell (Worthington Biochemicals, cat #8736)
Lysozyme: chicken egg white, Sigma cat #L6876; 50,000 U/mg; CAS 12650-88-3; molecular weight:
14.3 kD; solubility (H₂O) 10 mg/mL; stability - 1 month at 2-8° C. Standard: 25 μl of a 500,000 units (10 mg)/mL
(10 mM Tris-HCl) will typically lyse E. coli from >1 mL of culture media cell pellet resuspended
in 350 μl buffer (10 mM Tris HCl, pH 8.0, with 0.1 M NaCl, 1 mM EDTA, and 5% [w/v] Triton X-I00).
Typical incubation conditions for lysis are 30 min at 37° C.

Micrococcus lysodeikticus cell suspension was made by adding 9 mg Micrococcus lysodeikticus to 25 mL 10 mM Tris-HCl, pH 8.0 and mixing well. Lysozyme solution was prepared by adding 10 mg lysozyme in 1 mL 10 mM Tris-HCl, pH 8.0, and mixing well. Reaction buffer was 10 mM Tris-HCl, pH 8.0, with an alternative reaction buffer being 0.1 M KPO₄ pH 6.4.

A standard curve of the M. lysodeikticus was prepared. The lysozyme stock solution was diluted with the reaction buffer to create the following series: 10 mg/mL (undiluted); 5.0 mg/mL; 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL, and 0 mg/mL. The controls included 3 replicates of 194 μL M. lysodeikticus cell suspension plus 6 μL buffer; and 3 replicates of 200 μL buffer.

Analysis of samples included determining activity by monitoring the clearing of the cell suspension at 570 nm and determining the best fit to a standard curve. For a 200 μL assay, 180 μL M. lysodeikticus in reaction buffer was added to each well 1 to 12 of 3 rows. The reaction was started by adding 20 μL of each lysozyme dilution to each well in the triplicate series. The plate was immediately placed into the reader, and the changes in absorbance at 570 nm (OD₅₇₀) recorded. The number of reads may be 10-20 with second intervals. The plate readers velocity table contained data for reaction rate in mOD/min. This assay can be scaled by increasing each suspension proportionately (e.g., a 2 mL reaction is used for material strip analysis).

Analysis of the data included calculating the initial velocities for the recorded slopes: [mOD₅₄₀/min]/[slope standard curve (mOD/mg M. lysodeikticus]/[lysozyme].

TABLE 70
Assay Standardization

| | |
|---|---|
| Coupon Size | None |
| Test Organism | Micrococcus lysodeikticus |
| Contamination level | 2.5 × 10⁸ cells/mL |
| Assay Time | 4 hr |

TABLE 71
Standardization of Assay

| [Lysozyme], (μg/mL)^{a} | OD₅₇₀ | % Lysis |
|---|---|---|
| 0 | 0.3 | 0.00 |
| 0.78 | 0.26 | 13.33 |
| 1.56 | 0.07 | 76.67 |
| 3.13 | 0.02 | 93.33 |
| 6.25 | 0.005 | 98.33 |
| 12.5 | 0.005 | 98.33 |
| 25 | 0.011 | 96.33 |
| 50 | 0.065 | 78.33 |

^{a}μg/mL = ppm

The M. lysodeikticus assay as described can detect lytic activity down to the fractional to low ppm range. The rate of lysis, in suspension, is 32% (about 8.0×10⁷ cells) of the M. lysodeikticus suspension per μg lysozyme.

### Example 37

This Example demonstrates a spectrophotometric assay for antimicrobial paper with a lytic additive. Lysozyme was used as the lytic additive. Equipment and reagents that were used are shown in the table below.

TABLE 72
Equipment and Reagents

Equipment
Spectrophotometer (Thermo Multiskan Ascent Plate Reader)
Cuvettes (96-well assay plates)
Multi-channels and single-channel pipettes and tips
Reagents
Tris(hydroxymethyl)aminomethane hydrochloride (Tris-HCl): [Sigma,
cat # T3253, Molecular Formula: NH₂C(CH₂OH)₃•HCl,
Molecular Weight: 157.60, CAS Number 1185-53-1, pKa (25° C.) 8.1]
Micrococcus lysodeikticus cell (Worthington Biochemicals, cat #8736)
Lysozyme: chicken egg white, Sigma cat #L6876; 50,000 U/mg;
CAS 12650-88-3; molecular weight: 14.3 kD; solubility (H₂O) 10 mg/mL;
stability - 1 month at 2-8° C. Standard: 25 μl of a 500,000 units
(10 mg)/mL (10 mM Tris-HCl) will typically lyse E. coli from >1 mL
of culture media cell pellet resuspended in 350 μl buffer
(10 mM Tris HCl, pH 8.0, with 0.1 M NaCl, 1 mM EDTA, and
5% [w/v] Triton X-I00). Typical incubation conditions for lysis
are 30 min at 37° C.

Micrococcus lysodeikticus cell suspension was made by adding 9 mg M. lysodeikticus to 25 mL 10 mM Tris-HCl, pH 8.0 and mixing well. Lysozyme solution was prepared by adding 10 mg lysozyme in 1 mL 10 mM Tris-HCl, pH 8.0, and mixing well. Reaction buffer was 10 mM Tris-HCl, pH 8.0, with an alternative reaction buffer being 0.1 M KPO₄ pH 6.4. Antimicrobial paper coated with a coating comprising lysozyme and control paper was prepared in accordance with Example 35.

A standard curve of the M. lysodeikticus was prepared. The lysozyme stock solution was diluted with the reaction buffer to create the following series: 10 mg/mL (undiluted); 5.0 mg/mL; 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL and 0 mg/ml. The controls included 3 replicates of 194 μL M. lysodeikticus cell suspension plus 6 μL buffer; and 3 replicates of 200 μL buffer. Pipet tips used fitted the pipette (e.g., multichannel pipettes). The liquid level was correct in the tips, as air bubbles, etc may alter volume. Quality control and safety procedures were as described in Example 33.

Antimicrobial paper was cut into appropriately sized strips from both the antimicrobial and control paper. For a 5 mL assay in a 15 mL tube, standard sizes included 5×10 mm, 5×20 mm, and 5×40 mm. These strips could be combined to provide a desired step series.

Analysis of samples included determining activity by monitoring the clearing of the cell suspension at OD₅₇₀ and determining the best fit to a standard curve. For a 5 mL assay, M. lysodeikticus was added in reaction buffer to an OD₆₀₀ of 0.5. The reaction was started with the addition of the stripes. The tubes were immediately placed at 28° C. for a designated time (e.g., 4 hr and 24 hr). The absorbance at 570 nm was recorded.

Analysis of the data included calculating the initial velocities for the recorded slopes: [OD₆₀₀ min]/[slope standard curve (OD/mg M. lysodeikticus]/[lysozyme]

### Example 38

This Example demonstrates a biological assay for antimicrobial activity of paper strips comprising an antimicrobial enzyme additive against a microorganism. The antimicrobial enzyme additive comprised lysozyme, the microorganism used was vegetative, gram-positive M. lysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 73
Equipment and Reagents

Equipment:
Petri Plates
Reagents:
Nutrient Yeast Extract (NBY) NBY Soft Agar
Lysozyme: chicken egg white, Sigma cat #L6876; 50,000 U/mg;
CAS 12650-88-3; molecular weight: 14.3 kD; solubility (H₂O)
10 mg/mL; stability - 1 month at 2-8° C. Standard: 25 μl of
a 500,000 units (10 mg)/mL (10 mM Tris-HCl) will typically lyse
E. coli from >1 mL of culture media cell pellet resuspended
in 350 μl buffer (10 mM Tris HCl, pH 8.0, with 0.1 M NaCl,
1 mM EDTA, and 5% [w/v] Triton X-I00).
Typical incubation conditions for lysis are 30 min at 37° C.

Micrococcus lysodeikticus cell suspension was made by adding 9 mg Micrococcus lysodeikticus to NBY and mixing well, with OD₆₀₀ about 0.5. Antimicrobial paper coated with a latex coating comprising lysozyme and control paper was prepared in accordance with Example 35.

The assay include cutting appropriated sized strips of both antimicrobial and control papers (e.g., a. 10×10 mm, 20×20 mm, 40×40 mm, or 50×50 mm). 100 μL of the prepared M. lysodeikticus suspension was transferred to 15 mL tube containing 5 mL NBY Soft Agar, held molten at 55° C., and mixed well. Pipet tips used fitted the pipette (e.g., multichannel pipettes). The liquid level was correct in the tips, as air bubbles, etc may alter volume. The mixture was immediately poured over a prepared sterile agar plate, rotating the dish to completely cover the agar with the M. lysodeikticus overlay. The dish was covered and allowed to solidify on level surface. The prepared antimicrobial paper(s) were placed (face down) on the soft agar overlay. Coupon(s) up to 20×20 mm were able to be paired with a control on a single petri dish. The dishes were left at 28° C. overnight, and visually evaluated for a zone of clearance around the antimicrobial coupon(s) relative to the control. Quality control and safety procedures were as described in Example 33.

### Example 39

This Example demonstrates a biological assay for the antimicrobial activity of a paper strip comprising PrateCoat® against fungal spores. The assay was adapted from ASTM 02020-92, Method A, Standard Test for Mildew (Fungus) Resistance of Paper and Paperboard (Reapproved 2003). Equipment and reagents that were used are shown in the table below.

TABLE 74
Equipment and reagents

Equipment:
Petri Plates
Incubator
Autoclave
Preval Sprayer
Reagents:
Nutrient Yeast Extract (NBY)
NBY Soft Agar
Micrococcus lysodeikticus cell (Worthington Biochemicals, cat #8736)
ProteCoat ® was used at 125 mg ProteCoat ® per g coating,
dispensed as 250 mg ProteCoat ®, and resuspended in
2 mL dilute acetic acid solution as appropriate.

Fusarium oxysporium spores were prepared by maintaining cultures of Fusarium oxysporum f. sp. lycoperici race 1 (RM-1)[FOLRM-1 on Potato Dextrose Agar (PDA) slants. Microconidia of the Fusarium oxysporum f. sp. lycoperici, were obtained by isolating a small portion of an actively growing culture from a PDA plate and transferring to 50 ml a mineral salts medium FLC (Esposito and Fletcher, 1961). The culture was incubated with shaking (125 rpm) at 25° C. After 960 h the fungal slurry consisting of mycelia and microconidia were strained twice through eight layers of sterile cheese cloth to obtain a microconidial suspension. The microcondial suspension was then calibrated with a hemacytometer. All fungal inocula were tested for the absence of contaminating bacteria before their use in experiments. Antimicrobial paper coated with a latex coating comprising PrateCoat® and control paper was prepared in accordance with Example 35.

The assay procedure included: cutting appropriated sized strips of both antimicrobial and control papers (e.g., 40×40 mm or 50×50 mm); centering the strips on a sterile Potato Dextrose Agar plate, treated side up; diluting spores to 2×10³ per mL Potato Dextrose broth; transferring to a calibrated preval sprayer (i.e., dispense 50 μL per single pump action); dispersing spores in a hood onto the agar and paper surface with a single pump action (delivers approximately 100 spores to the area); covering and leaving at ambient conditions; and observing growth over several days, though time of assay will depend on organism. Pipet tips fitted the pipette (e.g., multichannel pipettes). The liquid level was correct in the tips, as air bubbles, etc may alter volume. Quality control and safety procedures were as described in Example 33.

### Example 40

This Example demonstrates a paper coating comprising an antimicrobial enzyme additive. The antimicrobial enzyme comprised a lysozyme. Assay standardization and data are shown in the following tables.

TABLE 75
Assay Enzymatic Additive-Lysozyme

| | |
|---|---|
| Example Techniques Used | Example 40 and 37 |
| Coupon Size | Variable, 200-600 mm² |
| Paper Age | 3 months |
| Test Organism | Micrococcus lysodeikticus |
| Contamination level | 2.5 × 10⁸ cells/mL |
| Assay Time | 4 and 24 hrs |

TABLE 76A
Test Strips and Data

| Paper | Paper coupon | Area | [lysozyme], |
|---|---|---|---|
| Type | (mm × mm) | (mm²) | μg |
| 0 | | 0 | |
| 0.2% | 5 × 40 | 200 | 8.76 |
| 1.0% | 5 × 40 | 200 | 38.80 |
| 2.0% | 5 × 40 | 200 | 92.80 |
| 2.0% | 5 × 40 + 5 × 10 | 250 | 116.00 |
| 2.0% | 5 × 40 + 5 × 20 | 300 | 139.20 |
| 2.0% | 5 × 40 + 5 × 40 | 400 | 185.00 |
| 2.0% | 5 × 40 + 5 × 40 + 5 × 10 | 450 | 208.80 |
| 2.0% | 5 × 40 + 5 × 40 + 5 × 20 | 500 | 232.00 |
| 2.0% | 5 × 40 + 5 × 40 + 5 × 40 | 600 | 278.40 |

TABLE 76B
Antimicrobial Strips and Data

| Paper | Paper coupon | 4 hrs | | 24 hrs | |
|---|---|---|---|---|---|
| Type | (mm × mm) | OD₅₇₀ | % Lysis | OD₅₇₀ | % Lysis |
| 0 | | 0.305 | 0.00 | 0.27 | 0.00 |
| 0.2% | 5 × 40 | 0.301 | 1.31 | 0.275 | −1.85 |
| 1.0% | 5 × 40 | 0.277 | 9.18 | 0.2 | 25.93 |
| 2.0% | 5 × 40 | 0.172 | 43.61 | 0.0015 | 99.44 |
| 2.0% | 5 × 40 + 5 × 10 | 0.099 | 67.54 | 0.001 | 99.63 |
| 2.0% | 5 × 40 + 5 × 20 | 0.136 | 55.41 | 0.0025 | 99.07 |
| 2.0% | 5 × 40 + 5 × 40 | 0.017 | 94.43 | 0.005 | 99.81 |
| 2.0% | 5 × 40 + 5 × 40 + 5 × 10 | 0.023 | 92.46 | 0.001 | 99.63 |
| 2.0% | 5 × 40 + 5 × 40 + 5 × 20 | 0.024 | 92.13 | 0.001 | 99.63 |
| 2.0% | 5 × 40 + 5 × 40 + 5 × 40 | 0.015 | 95.08 | 0.0015 | 99.44 |

The rate of lysis upon contact with a coupon cut from antimicrobial treated paper, is approximately 0.5% (1.35×10⁷ cells) per μg lysozyme. This corresponds to a reduction in activity, per μg of lysozyme, of approximately 65% over that observed in suspension. Treated papers of identical size with antimicrobial loadings of 0.2%, 1.0% and 2.0%, demonstrated antimicrobial function. The antimicrobial concentration on a per unit of area for those loadings, is provided in the following table.

TABLE 77
Antimicrobial concentration per unit area

| | | | Lysozyme | | |
|---|---|---|---|---|---|
| Paper | Coating (gsm) | % lysozyme | g/m² | μg/m² | μg/mm² |
| A | 21.9 | 0.2% | 0.0438 | 4.38 × 10⁻⁸ | 0.0438 |
| B | 19.4 | 1.0% | 0.194 | 1.94 × 10⁻⁷ | 0.194 |
| C | 23.2 | 2.0% | 0.464 | 4.64 × 10⁻⁷ | 0.464 |

### Example 41

This Example qualitatively demonstrates an antimicrobial enzyme additive combined with an antimicrobial peptide additive to provide antimicrobial functionality to a paper coating formulation. An adaptation of ASTM 02020-92 was used as the assay to demonstrate the growth of a microorganism in a petri dish was inhibited by contact with the treated paper. The antimicrobial enzyme additive comprised lysozyme, and the antimicrobial peptide additive comprised ProteCoat® Reactive Surfaces, Ltd.; also described in U.S. patent application Ser. Nos. 10/884,355; 11/368,086; and 11/865,514, each incorporated by reference).

The spectrophotometric lysozyme assay uses Micrococcus lysodeikticus bacterial cells as a substrate, and measures the change in the turbidity of the cell suspension as described in Example 36 and Example 37. The efficacy of an antimicrobial peptide (e.g., ProteCoat™) may be monitored biologically. Though the contemplated mechanism of action for an antimicrobial or antifouling peptide is similar, i.e. disruption of the structural components of the microbial cell, the cell wall may remain relatively intact. As an antifungal or antimicrobial peptide's biocidal or biostatic activity inhibits the cell, the cell may not lyse for detection of a change in turbidity. Biological assay conditions are shown in the table below.

TABLE 78
Enzymatic Additive-Lysozyme (Qualitative)

| | |
|---|---|
| Example Techniques Used | Example 38 |
| Coupon Size | 100 mm² |
| Paper Age | 3 months |
| Test Organism | Micrococcus lysodeikticus |
| Growth Conditions | 28° C. |

A zone of clearing was seen around the antimicrobial paper in contact with a petri dish covered by M. lysodeikticus, whereas the control paper had no such zone. The coupon of paper was about half the size of the smallest coupons in the quantitative M. lysodeikticus assay, yet growth inhibition was seen.

Assay conditions for Fusarium oxysporum is shown at the table below.

TABLE 79
Enzymatic Additive-ProteCoat ® (Qualitative)

| | |
|---|---|
| Example Techniques Used | Example 39 |
| Coupon Size | 40 × 40 mm |
| Paper Age | 3 months |
| Test Organism | Fusarium oxysporum |
| Contamination level | 100 spore, aerosol delivery |
| Growth Conditions | Ambient |

Overgrowth of both test and control ProteCoat® paper by the fungus, Fusarium oxysporium, was observed. The developmental state of the mycelium on the antimicrobial paper was retarded over that seen in the control paper, indicative of biostatic, and possibly biocide activity.

### Example 42

This Example demonstrates synergism between an antimicrobial enzyme additive combined with an antimicrobial peptide additive in a coating applied to papers, and to demonstrate antimicrobial activity of a paper comprising the antimicrobial peptide. The antimicrobial enzyme additive comprised lysozyme, and the antimicrobial peptide additive comprised ProteCoat® (Reactive Surfaces, Ltd.; also described in U.S. patent application Ser. Nos. 10/884,355; 11/368,086; and 11/865,514, each incorporated by reference). Assay conditions are shown at the tables below.

TABLE 80
Enzymatic Additive- 2% Lysozyme + 0.5% ProteCoat ® (Titration Assay)

| | |
|---|---|
| Example Techniques Used | Example 37 |
| Coupon Size | Variable, 0-400 mm² |
| Paper Age | 3 months |
| Test Organism | Micrococcus lysodeikticus |
| Contamination level | 2.5 × 10⁸ cells/mL |
| Assay Time | 3 and 20 hrs |

TABLE 81A
Activity in Treated Papers

| | | Area | Lysozyme | | ProteCoat ® | |
|---|---|---|---|---|---|---|
| Paper | Strips (mm × mm) | (mm²) | mg | μg/mL | mg | μg/mL |
| 2% Lysozyme | | 0 | 0 | | | |
| | 5 × 5 | 25 | 11.60 | 2.90 | 0.00 | 0.00 |
| | 5 × 10 | 50 | 23.20 | 5.80 | 0.00 | 0.00 |
| | 5 × 20 | 100 | 46.40 | 11.60 | 0.00 | 0.00 |
| | 5 × 40 | 200 | 92.80 | 23.20 | 0.00 | 0.00 |
| | 5 × 40 + 5 × 5 | 225 | 104.40 | 26.10 | 0.00 | 0.00 |
| | 5 × 40 + 5 × 10 | 250 | 116.00 | 29.00 | 0.00 | 0.00 |
| | 5 × 40 + 5 × 20 | 300 | 139.20 | 34.80 | 0.00 | 0.00 |
| | 5 × 40 + 5 × 40 | 400 | 185.60 | 46.40 | 0.00 | 0.00 |
| 2% Lysozyme + | | 0 | | | | |
| 0.5% | 5 × 5 | 25 | 11.60 | 2.90 | 2.90 | 0.73 |
| ProteCoat ® | 5 × 10 | 50 | 23.20 | 5.80 | 5.80 | 1.45 |
| | 5 × 20 | 100 | 46.40 | 11.60 | 11.60 | 2.90 |
| | 5 × 40 | 200 | 92.80 | 23.20 | 23.20 | 5.80 |
| | 5 × 40 + 5 × 5 | 225 | 104.40 | 26.10 | 26.10 | 6.53 |
| | 5 × 40 + 5 × 10 | 250 | 116.00 | 29.00 | 29.00 | 7.25 |
| | 5 × 40 + 5 × 20 | 300 | 139.20 | 34.80 | 34.80 | 8.70 |
| | 5 × 40 + 5 × 40 | 400 | 185.60 | 46.40 | 46.40 | 11.60 |

TABLE 81B
Activity in Treated Papers

| | | Area | 3 hrs | | 20 hrs | |
|---|---|---|---|---|---|---|
| Paper | Strips (mm × mm) | (mm²) | OD₆₀₀ | % Lysis | OD₆₀₀ | % Lysis |
| 2% Lysozyme | | 0 | 0.266 | 0.00 | 0.258 | 0.00 |
| | 5 × 5 | 25 | 0.259 | 2.63 | 0.25 | 3.10 |
| | 5 × 10 | 50 | 0.259 | 2.63 | 0.23 | 10.85 |
| | 5 × 20 | 100 | 0.256 | 3.76 | 0.145 | 43.80 |
| | 5 × 40 | 200 | 0.228 | 14.29 | 0.038 | 85.27 |
| | 5 × 40 + 5 × 5 | 225 | 0.199 | 25.19 | 0.019 | 92.64 |
| | 5 × 40 + 5 × 10 | 250 | 0.148 | 44.36 | 0.011 | 95.74 |
| | 5 × 40 + 5 × 20 | 300 | 0.177 | 33.46 | 0.013 | 94.96 |
| | 5 × 40 + 5 × 40 | 400 | 0.09 | 66.17 | 0.012 | 95.35 |
| 2% Lysozyme + | | 0 | 0.266 | 0.00 | 0.258 | 0.00 |
| 0.5% | 5 × 5 | 25 | 0.255 | 4.14 | 0.23 | 10.85 |
| ProteCoat ® | 5 × 10 | 50 | 0.248 | 6.77 | 0.057 | 77.91 |
| | 5 × 20 | 100 | 0.237 | 10.90 | 0.016 | 93.80 |
| | 5 × 40 | 200 | 0.195 | 26.69 | 0.012 | 95.35 |
| | 5 × 40 + 5 × 5 | 225 | 0.199 | 25.19 | 0.012 | 95.35 |
| | 5 × 40 + 5 × 10 | 250 | 0.15 | 43.61 | 0.012 | 95.35 |
| | 5 × 40 + 5 × 20 | 300 | 0.124 | 53.38 | 0.01 | 96.12 |
| | 5 × 40 + 5 × 40 | 400 | 0.031 | 88.35 | 0.012 | 95.35 |

The concentration of lysozyme in the papers corresponded to between 2 and 50 ppm, whereas ProteCoat® was between 0.5 and 12 ppm. The comparison of lysis between the 2% lysozyme paper, and the combined paper which contained 2% lysozyme and 0.5% ProteCoat® indicates synergism between the additives. For example, the 100 mm² coupon size exhibited 44% lysis, whereas the combined paper exhibited 93%. This is an observed/expected (93/44+0) of 2.1, indicative of significant synergism. To further demonstrate this activity, the assay was repeated by titrating the 2% lysozyme paper with individual swaths of 2.5% ProteCoat® paper. 5×10, 5×20, and 5×40 mm² lysozyme paper strips with increasing amount of Protecoat® paper were added to tubes in 4 ml total volume 2.5×10⁸ Micrococcus cells/ml. The assay conditions are shown at the tables below.

TABLE 82
Enzymatic Additive-2% Lysozyme & 2.5% ProteCoat ® (Titration)

| | |
|---|---|
| Example Techniques Used | Example 37 |
| Coupon Size | Variable |
| Lysozyme | 0-200 mm² |
| ProteCoat ® | 0-200 mm² |
| Paper Age | 3 months |
| Test Organism | Micrococcus lysodeikticus |
| Contamination level | 2.5 × 10⁸ cells/mL |
| Assay Time | 4 and 22 hrs |

TABLE 83
Activity of Protecoat ® paper with 50, 100 and 200 mm² Lysozyme
paper against Micrococcus lysodeikticus

| | | Square area | Square | | |
|---|---|---|---|---|---|
| | Strips (mm × | (mm²) | area (mm²) | [lysozyme] | [Protecoat ®] |
| Paper | mm) | Lysozyme | Protecoat ® | (ug/ml) | (ug/ml) |
| Control | 0 | 0 | 0 | 0 (0) | 0 (0) |
| 2% Lysozyme | 5 × 10 | 50 | 0 | 23.2 (5.8) | 0 (0) |
| 2.5% | 5 × 5 | 50 | 25 | 23.2 (5.8) | 15 (3.75) |
| Protecoat ® | 5 × 10 | 50 | 50 | 23.2 (5.8) | 30 (7.5) |
| | 5 × 20 | 50 | 100 | 23.2 (5.8) | 60 (15) |
| | 5 × 40 | 50 | 200 | 23.2 (5.8) | 120 (30) |
| | 5 × 40 × 2 | 50 | 400 | 23.2 (5.8) | 240 (60) |
| Control | 0 | 0 | 0 | 0 (0) | 0 (0) |
| 2% Lysozyme | 5 × 20 | 100 | 0 | 46.4 (11.6) | 0 (0) |
| 2.5% | 5 × 5 | 100 | 25 | 46.4 (11.6) | 15 (3.75) |
| Protecoat ® | 5 × 10 | 100 | 50 | 46.4 (11.6) | 30 (7.5) |
| | 5 × 20 | 100 | 100 | 46.4 (11.6) | 60 (15) |
| | 5 × 40 | 100 | 200 | 46.4 (11.6) | 120 (30) |
| | 5 × 40 × 2 | 100 | 400 | 46.4 (11.6) | 240 (60) |
| 2% Lysozyme | 5 × 40 | 200 | 0 | 92.8 (23.2) | 0 (0) |
| 2.5% | 5 × 5 | 200 | 25 | 92.8 (23.2) | 15 (3.75) |
| Protecoat ® | 5 × 10 | 200 | 50 | 92.8 (23.2) | 30 (7.5) |
| | 5 × 20 | 200 | 100 | 92.8 (23.2) | 60 (15) |
| | 5 × 40 | 200 | 200 | 92.8 (23.2) | 120 (30) |
| | 5 × 40 × 2 | 200 | 400 | 92.8 (23.2) | 240 (60) |

An example of a calculation for the lysozyme content in 2% lysozyme paper was: 23.2×2% g/m²=0.464 g/m²=0.464 μg/mm². An example of a calculation for the Protecoat® content in 2.5% Protecoat® paper was: 23.9×2.5% g/m²=0.60 g/m²=0.60 μg/mm².

TABLE 84
Activity of Protecoat ® paper with 50, 100 and 200 mm² Lysozyme
paper against Micrococcus lysodeikticus

| | Strips | 4 hrs | | 23 hrs | |
|---|---|---|---|---|---|
| Paper | (mm × mm) | OD₆₀₀ | % Lysis | OD₆₀₀ | % Lysis |
| Control | 0 | 0.278 | 0 | 0.276 | 0 |
| 2% Lysozyme | 5 × 10 | 0.269 | 3.24 | 0.206 | 25.36 |
| 2.5% | 5 × 5 | 0.264 | 5.04 | 0.235 | 14.86 |
| Protecoat ® | 5 × 10 | 0.268 | 3.60 | 0.213 | 22.83 |
| | 5 × 20 | 0.269 | 3.24 | 0.197 | 28.62 |
| | 5 × 40 | 0.266 | 4.32 | 0.172 | 37.68 |
| | 5 × 40 × 2 | 0.24 | 13.67 | 0.027 | 90.22 |
| Control | 0 | 0.254 | 0 | 0.229 | 0 |
| 2% Lysozyme | 5 × 20 | 0.224 | 11.81 | 0.026 | 88.65 |
| 2.5% | 5 × 5 | 0.22 | 13.39 | 0.023 | 89.96 |
| Protecoat ® | 5 × 10 | 0.204 | 19.69 | 0.013 | 94.32 |
| | 5 × 20 | 0.212 | 16.54 | 0.019 | 91.70 |
| | 5 × 40 | 0.178 | 29.92 | 0.014 | 93.89 |
| | 5 × 40 × 2 | 0.194 | 23.62 | 0.027 | 88.21 |
| 2% Lysozyme | 5 × 40 | 0.203 | 20.08 | 0.019 | 91.70 |
| 2.5% | 5 × 5 | 0.181 | 28.74 | 0.009 | 96.07 |
| Protecoat ® | 5 × 10 | 0.175 | 31.10 | 0.01 | 95.63 |
| | 5 × 20 | 0.165 | 35.04 | 0.012 | 94.76 |
| | 5 × 40 | 0.128 | 49.61 | 0.012 | 94.76 |
| | 5 × 40 × 2 | 0.145 | 42.91 | 0.019 | 91.70 |

TABLE 85A
% Lysis (relative to control without Protecoat ® added) at
given time

| | 4 hr | | |
|---|---|---|---|
| Square Area | 50 mm² | 100 mm² | 200 mm² |
| (mm²) of | Lysozyme | Lysozyme | Lysozyme |
| Protecoat ® paper | paper | paper | paper |
| 0 | 3.24 | 11.81 | 20.08 |
| 25 | 5.04 | 13.39 | 28.74 |
| 50 | 3.60 | 19.69 | 31.10 |
| 100 | 3.24 | 16.54 | 35.04 |
| 200 | 4.32 | 29.92 | 49.61 |
| 400 | 13.67 | 23.62 | 42.91 |

TABLE 85B
% Lysis (relative to control without Protecoat ® added) at
given time

| | 22 hr | | |
|---|---|---|---|
| Square Area | 50 mm² | 100 mm² | 200 mm² |
| (mm²) of | Lysozyme | Lysozyme | Lysozyme |
| Protecoat ® paper | paper | paper | paper |
| 0 | 25.36 | 88.65 | 91.70 |
| 25 | 14.86 | 89.96 | 96.07 |
| 50 | 22.83 | 94.32 | 95.63 |
| 100 | 28.62 | 91.70 | 94.76 |
| 200 | 37.68 | 93.89 | 94.76 |
| 400 | 90.22 | 88.21 | 91.70 |

The assay was repeated by titrating the 2% lysozyme paper with individual swaths of 2.5% ProteCoat® paper. Lysozyme in technical papers added to an assay at concentrations greater than 10 ppm exhibited antimicrobial activity in the M. lysodeikticus assay. Lysozyme at approximately 5 ppm in the assay did not exhibit significant antimicrobial activity over the course of the assay (20 hrs). The addition of ProteCoat® papers, with between 3 and 60 ppm ProteCoat® to the assay significantly enhanced the lytic activity of lysozyme, or possibly the reverse. This was also true with the 5 ppm lysozyme, in which the lytic activity was doubled by the addition of between 3 and 60 ppm ProteCoat® to the assay. The peptide additive may be enhancing the activity of the enzyme, or the enzyme enhancing the activity of the peptide, or both, to produce these results.

### Example 43

This Example demonstrates a spectrophotometric assay for an antimicrobial coating with a lytic additive. The lytic additive comprised a lysozyme. The antimicrobial coatings were created using acrylic latex, commercially available paints. Equipment and reagents that were used are shown in the table below.

TABLE 86
Equipment and Reagents

Equipment
Spectrophotometer (Thermo Multiskan Ascent Plate Reader)
Cuvettes (96-well assay plates)
Multi-channels and single-channel pipettes and tips
Reagents
Tris(hydroxymethyl)aminomethane hydrochloride (Tris-HCl): [Sigma,
cat # T3253, Molecular Formula: NH₂C(CH₂OH)₃•HCl,
Molecular Weight: 157.60, CAS Number 1185-53-1, pKa (25° C.) 8.1]
Micrococcus lysodeikticus cell (Worthington Biochemicals, cat #8736)
Lysozyme: chicken egg white {Sigma cat #L6876; 50,000 U/mg;
CAS 12650-88-3; molecular weight: 14.3 kD; solubility (H₂O) 10 mg/mL;
stability - 1 month at 2-8° C. Standard: 25 μl of a 500,000 units
(10 mg)/mL (10 mM Tris-HCl) will typically lyse E. coli from >1 mL of
culture media cell pellet resuspended in 350 μl buffer (10 mM Tris HCl,
pH 8.0, with 0.1 M NaCl, 1 mM EDTA, and 5% [w/v] Triton X-I00).
Typical incubation conditions for lysis are 30 min at 37° C.}

A Micrococcus lysodeikticus cell suspension was made by adding 1.5 mg Micrococcus lysodeikticus to 1 mL 10 mM Tris pH 8.0 and mixing well. A lysozyme solution was prepared by adding 10 mg lysozyme in 1 mL ddH₂O, and mixing well.

The lysozyme stock solution was mixed into Sherwin Williams Acrylic (SW) or Glidden latex paint (1 part water:7 part paint). 4 mil, 6 mil, and 8 mil free films were created from Sherwin Williams paint comprising a lysozyme, a Glidden paint comprising a lysozyme, and controls for both. The plate controls included 3 replicates of 50 μL M. lysodeikticus cell suspension plus 50 μL buffer; and 3 replicates of 100 μL buffer. Pipet tips used fitted the pipette (e.g., multichannel pipettes). The liquid level was correct in the tips, as air bubbles, etc may alter volume. Quality control and safety procedures were as described in Example 33.

The antimicrobial films were cut into appropriately sized strips from both the antimicrobial and control coating. For a 5 mL assay in a 15 mL tube, standard size was 1×1 cm.

Analysis of samples included determining activity by monitoring the clearing of the cell suspension at OD₄₀₅ and determining the best fit to a standard curve. The reaction was started with the addition of 5 ml of the M. lysodeikticus stock. The tubes were immediately placed on a rocker for 3 hr; 100 μl samples were taken at 3 hr, and the absorbance at 405 nm was recorded.

TABLE 87
Sample Lysis Averages and Deviations

| | | Avg. % | |
|---|---|---|---|
| | | Lysis at | Standard |
| Sample | | 3 hr | Deviation |
| SW Control | 4 mils | 11.1057 | 0.5752 |
| | 6 mils | 12.2932 | 0.3812 |
| | 8 mils | 12.2802 | 0.5752 |
| SW Lysozyme | 4 mils | 65.0651 | 1.3638 |
| | 6 mils | 74.5744 | 3.8272 |
| | 8 mils | 84.2325 | 4.1432 |
| Glidden Control | 4 mils | 4.8514 | 0.4912 |
| | 6 mils | 5.1005 | 0.0569 |
| | 8 mils | 5.1749 | 0.6266 |
| Glidden | 4 mils | 18.3760 | 0.5846 |
| Lysozyme | 6 mils | 23.1840 | 3.6201 |
| | 8 mils | 29.1666 | 1.9095 |

Analysis of the data included calculating the initial velocities for the recorded slopes: [OD₄₀₅ min]/[slope standard curve (OD/mg M. lysodeikticus]/[lysozyme].

### Example 44

This Example demonstrates a biological assay for antimicrobial activity of coatings comprising an antimicrobial enzyme additive against a microorganism. The antimicrobial enzyme additive comprised lysozyme, the microorganism used comprised vegetative, gram-positive M. lysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 88
Equipment and Reagents

Equipment:
Petri Plates
Reagents:
Luria Broth Agar (LBA)
Lysozyme: chicken egg white, Sigma cat #L6876; 50,000 U/mg; CAS 12650-88-3; molecular weight: 14.3 kD; solubility (H₂O) 10 mg/mL; stability - 1 month at 2-8° C.
Standard: 25 μl of a 500,000 units (10 mg)/mL (10 mM Tris-HCl) will typically lyse E. coli from >1 mL of culture media cell pellet resuspended in 350 μl buffer (10 mM Tris HCl, pH 8.0, with 0.1 M NaCl, 1 mM EDTA, and 5% [w/v] Triton X-I00).
Typical incubation conditions for lysis are 30 min at 37° C.

A Micrococcus lysodeikticus cell suspension was made by adding 1.5 mg M. lysodeikticus to 10 mM Tris, pH 8.0, and mixing well. A lawn of M. lysodeikticus was generated by spreading 200 μl of this suspension onto a LBA plate, using a glass spreading rod. An antimicrobial latex coating comprising lysozyme and a control film was prepared in accordance with Example 43.

The assay include cutting appropriated sized strips of both antimicrobial and control latex films (e.g., a 1×1 cm). In triplicate the free films are carefully placed onto the surface of the petri dishes spaced out equally. This procedure was repeated for each of the paint film types/thicknesses.

The paint films comprising a lysozyme were active in lysing M. lysodeikticus, producing circular zones of clearing. The difference in Zone of Clearing Diameter between the different thicknesses of film was deemed negligible.

TABLE 89
Diameter (cm) of Zones of Clearing

| Sample | 4 mils | 6 mils | 8 mils |
|---|---|---|---|
| Glidden Lysozyme | 2.8 | 2.8 | 2.8 |
| | 2.8 | 2.9 | 2.8 |
| | 2.7 | 2.9 | 2.9 |
| Glidden Control | 0 | 0 | 0 |
| | 0 | 0 | 0 |
| | 0 | 0 | 0 |
| Sherwin Williams | 2.1 | 1.9 | 2.2 |
| Lysozyme | 2.1 | 1.9 | 1.9 |
| | 2 | 2 | 1.8 |
| Sherwin Williams | 0 | 0 | 0 |
| Lysozyme | 0 | 0 | 0 |
| | 0 | 0 | 0 |

### Example 45

This Example demonstrates a qualitative biological assay for survivability of an antimicrobial latex coating comprising an antimicrobial enzyme additive against a microorganism. The antimicrobial enzyme additive comprised lysozyme, the microorganism used comprised vegetative, gram-positive M. lysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 90
Equipment and Reagents

Equipment:
Petri Plates
Reagents:
Luria Broth Agar (LBA)
Lysozyme: chicken egg white, Sigma cat #L6876; 50,000 U/mg; CAS 12650-88-3; molecular weight: 14.3 kD; solubility (H₂O) 10 mg/mL; stability - 1 month at 2-8° C.
Standard: 25 μl of a 500,000 units (10 mg)/mL (10 mM Tris-HCl) will typically lyse E. coli from >1 mL of culture media cell pellet resuspended in 350 μl buffer (10 mM Tris HCl, pH 8.0, with 0.1 M NaCl, 1 mM EDTA, and 5% [w/v] Triton X-I00).
Typical incubation conditions for lysis are 30 min at 37° C.

A Micrococcus lysodeikticus cell suspension was made by adding 1.5 mg M. lysodeikticus to 10 mM Tris, pH 8.0, and mixing well. A lawn of M. lysodeikticus was generated by spreading 200 μl of this suspension onto a LBA plate, using a glass spreading rod.

The paint formulations that were prepared included a Sherwin-Williams Acrylic Latex or a Glidden Acrylic Latex as controls (no additive), and both a Sherwin-Williams Acrylic Latex or a Glidden Acrylic Latex comprising 10 mg/mL Lysozyme (ddH2O). Each paint was made by adding 1 part additive to 7 parts paint, and then mixed with a glass stirring rod and a paint mixer. Each film was immediately drawn onto polypropylene surfaces with a thickness of 4 mil, 6 mil, and 8 mil. Cure time was 24 days. Materials for assay were generated from the polypropylene surface as 1 cm² free films.

The assay include cutting appropriately sized strips of both antimicrobial and control latex films (e.g., a 1×1 cm). In triplicate the free films were carefully placed onto the surface of the petri dishes spaced out equally. This procedure was repeated for each of the paint film types/thicknesses.

After 24 hrs incubation, the diameter of the zones of clearing was measured for each film. Using sterile tweezer, the films were removed and transfer to a new LBA plate spread with M. lysodeikticus in the same orientation as the plates the films were removed from. Repeat the procedure of measuring the zones of clearing through transfer to a new plate every day for 5 days.

TABLE 91
Average Diameter (cm) of Zones of Clearing

| | | | Standard | | Standard | | Standard |
|---|---|---|---|---|---|---|---|
| | | 4 mils | Deviation | 6 mils | Deviation | 8 mils | Deviation |
| Day 1 | Glidden Control | N/A | N/A | N/A | N/A | 0 | 0 |
| | Glidden | 2.5667 | 0.0577 | 2.5333 | 0.0577 | 2.7000 | 0.0000 |
| | Lysozyme | | | | | | |
| Day 2 | Glidden Control | N/A | N/A | N/A | N/A | 0 | 0 |
| | Glidden | 2.0000 | 0.0000 | 2.0000 | 0.0000 | 2.2000 | 0.0000 |
| | Lysozyme | | | | | | |
| Day 3 | Glidden Control | N/A | N/A | N/A | N/A | 0 | 0 |
| | Glidden | 1.4667 | 0.0577 | 1.6667 | 0.0577 | 1.9000 | 0.0000 |
| | Lysozyme | | | | | | |
| Day 4 | Glidden Control | N/A | N/A | N/A | N/A | 0 | 0 |
| | Glidden | 1.4333 | 0.1155 | 1.5667 | 0.0577 | 1.8000 | 0.0000 |
| | Lysozyme | | | | | | |
| Day 5 | Glidden Control | N/A | N/A | N/A | N/A | 0 | 0 |
| | Glidden | 1.2667 | 0.0577 | 1.4500 | 0.0707 | 1.6333 | 0.0577 |
| | Lysozyme | | | | | | |

¹N/A in this chart just means not available/not applicable.

There were no 4 mil or 6 mil controls tested due to a limited LBA plate supply, though 8 mil control films were tested. The standard deviations for the 8 mil controls to 0, because all 3 controls produced a 0 cm zone of clearing in each case.

The paint films comprising lysozyme were active in lysing M. lysodeikticus, producing circular zones of clearing, for five cycles of contaminant control. The difference in Zone of Clearing Diameter between the different thicknesses of each film appeared negligible.

### Example 46

This Example demonstrates a sulfatase's activity in free-films using a plate reader. Equipment and reagents used are shown in the table below.

TABLE 92
Equipment and Reagents

Equipment
Plate Reader
96-well plate
2 ml microtubes
Reagents
Sulfatase from Aerobacter aerogenes (Sigma Cat# S1629-50UN)
Potassium 4-Nitrophenyl sulfate (MW 257.27; Sigma Cat# N3877)
Trizma base (Sigma Cat# T1503)

Samples preparation procedure included preparing: 14.5 mM potassium 4-nitrophenyl sulfate in isopropyl alchohol; and 200 mM TRIS, adjusted to pH 7.1 with HCl.

The paint formulations that were prepared included a Sherwin-Williams Acrylic Latex control (no additive), and a Sherwin-Williams Acrylic Latex comprising sulfatase. 63 enzyme units of sulfatase was admixed with 1 part water, then added to 7 parts paint. Each paint was mixed with a glass stirring rod and a paint mixer. Each film was immediately drawn onto polypropylene surfaces with a thickness of 8 mil. Cure time was 24 hours. Materials for assay were generated from the polypropylene surface as 3 cm² free films.

The plate reader assay included: cutting free films into appropriate size pieces; adding 1350 uL 200 mM TRIS into each microtube; adding 150 uL of 14.5 mM potassium 4-nitrophenyl sulfate to each tube; taking the 0 time sample; then adding the free films to the tubes, with the control sample being free film with no sulfatase. Quality control and safety procedures were as described in Example 33, including use of a hood for material handling as appropriate.

Analysis included: taking 100ul at the appropriate time points from each microtube and reading the absorbance at 405 nm; and determining the initial rate slope by plotting absorbance vs. time to calculate sulfatase activity.

TABLE 93A
Absorbance at 405 nm

| Time | Blank | | |
|---|---|---|---|
| 0 | 0.0410 | 0.0408 | 0.0401 |
| 15 | 0.0414 | 0.0409 | 0.0408 |
| 30 | 0.0411 | 0.0400 | 0.0410 |
| 60 | 0.0405 | 0.0410 | 0.0410 |
| 120 | 0.0428 | 0.0409 | 0.0412 |
| Slope | 0.0000 | 0.0000 | 0.0000 |

TABLE 93B
Absorbance at 405 nm

| Time | 3 cm × 1 cm Control | | | 3 cm × 1 cm Enzyme | | |
|---|---|---|---|---|---|---|
| 0 | 0.0410 | 0.0408 | 0.0401 | 0.0410 | 0.0408 | 0.0401 |
| 15 | 0.0420 | 0.0408 | 0.0407 | 0.0595 | 0.0592 | 0.0607 |
| 30 | 0.0450 | 0.0414 | 0.0413 | 0.0800 | 0.0819 | 0.0818 |
| 60 | 0.0421 | 0.0448 | 0.0500 | 0.1243 | 0.1307 | 0.1291 |
| 120 | 0.0415 | 0.0422 | 0.0430 | 0.2024 | 0.2138 | 0.2159 |
| Slope | 0.0000 | 0.0000 | 0.0000 | 0.0014 | 0.0015 | 0.0015 |

TABLE 94A
Average Absorbance at 405 nm

| | Absorbance Average | | |
|---|---|---|---|
| Time | Blank | Control 3 cm² | Sulfatase 3 cm² |
| 0 | 0.0406 | 0.0406 | 0.0406 |
| 15 | 0.0410 | 0.0412 | 0.0598 |
| 30 | 0.0407 | 0.0426 | 0.0812 |
| 60 | 0.0408 | 0.0456 | 0.1280 |
| 120 | 0.0416 | 0.0422 | 0.2107 |

TABLE 94B
Average Absorbance at 405 nm Standard Deviations

| | Absorbance Standard Deviation | | |
|---|---|---|---|
| Time | Blank | Control 3 cm² | Sulfatase 3 cm² |
| 0 | 0.0005 | 0.0005 | 0.0005 |
| 15 | 0.0003 | 0.0007 | 0.0008 |
| 30 | 0.0006 | 0.0021 | 0.0011 |
| 60 | 0.0003 | 0.0040 | 0.0033 |
| 120 | 0.0010 | 0.0008 | 0.0073 |

TABLE 95
Absorbance vs. Time Slope Activity Data

| | Slope | U | U | U |
|---|---|---|---|---|
| Sample | (A/min) | (umol/min) | Average | Deviation |
| Blank | 0.0000 | 0.0028 | 0.0016 | 0.0012 |
| | 0.0000 | 0.0005 | | |
| | 0.0000 | 0.0015 | | |
| Control | 0.0000 | −0.0009 | 0.0036 | 0.0045 |
| 3 cm² | 0.0000 | 0.0038 | | |
| | 0.0000 | 0.0080 | | |
| Sulfatase | 0.0014 | 0.2971 | 0.3133 | 0.0141 |
| 3 cm² | 0.0015 | 0.3200 | | |
| | 0.0015 | 0.3229 | | |

### Example 47

This Example demonstrates a phosphodiesterase I assay using a plate reader. The equipment and reagents used are shown in the table below.

TABLE 96
Equipment and reagents

Equipment
Plate Reader
96-well plate
Reagents
Phosphodiesterase I from Crotalus adamanteus Venom
(Worthington Cat# LS003926)
Thymidine 5-monophosphate p-nitrophenyl ester sodium salt
(MW 465.3; Sigma Cat# T4510)
Trizma base (Sigma Cat# T1503)

Samples prepared included: 14.5 mM Thymidine 5-monophosphate p-nitrophenyl ester sodium salt in ddH₂O; a 124 U/ml ddH₂O enzyme solution; and 200 mM TRIS (adjusted to pH 7.1 with HCl).

The plate reader assay comprised: diluting enzyme solution 1:1 and 1:3; adding 16 ul of each enzyme dilution in triplicate into a 96-well plate, with a control sample prepared by adding 16 ul ddH₂O; adding 24 ul ddH₂O into each well; adding 50 ul 200 mM TRIS to each well; and adding 10 uL of 14.5 mM Thymidine 5-monophosphate p-nitrophenyl ester sodium salt in ddH₂O to each well. Quality control and safety procedures were as described in Example 33, including use of a hood for material handling as appropriate.

The analysis included: taking 500 readings every 10 seconds at 405 nm; and determining the initial rate slope by plotting absorbance vs. time to calculate phosphodiesterase I activity. Summary results are below.

TABLE 97
Phosphodiesterase Activity

| | Slope | U | U | U |
|---|---|---|---|---|
| Sample | (A/min) | (umol/min) | Average | Deviation |
| 2U | 0.1069 | 23.39 | 20.48 | 2.58 |
| | 0.0895 | 19.60 | | |
| | 0.0844 | 18.47 | | |
| 1U | 0.0764 | 16.73 | 15.27 | 1.69 |
| | 0.0715 | 15.64 | | |
| | 0.0613 | 13.42 | | |

TABLE 98
Phosphodiesterase Activity

| | Slope | U | U | U |
|---|---|---|---|---|
| Sample | (A/min) | (umol/min) | Average | Deviation |
| 0.5U | 0.0508 | 11.12 | 10.62 | 0.54 |
| | 0.0488 | 10.69 | | |
| | 0.0459 | 10.05 | | |
| Control | −0.0002 | −0.04 | −0.04 | 0.03 |
| | −0.0004 | −0.08 | | |
| | −0.0001 | −0.01 | | |

### Example 48

This Example demonstrates a phosphodiesterase I activity assay in free-films using a plate reader.

TABLE 99
Equipment and reagents

Equipment
Plate Reader
96-well plate
2 ml microtubes
Reagents
Phosphodiesterase I from Crotalus adamanteus Venom
(Worthington Cat# LS003926)
Thymidine 5-monophosphate p-nitrophenyl ester sodium salt
(MW 465.3; Sigma Cat# T4510)
Trizma base (Sigma Cat# T1503)

Samples prepared included: 14.5 mM Thymidine 5-monophosphate p-nitrophenyl ester sodium salt in ddH₂O; and 200 mM TRIS (adjusted to pH 7.1 with HCl).

The paint formulations that were prepared included a Sherwin-Williams Acrylic Latex control (no additive), and a Sherwin-Williams Acrylic Latex comprising phosphodiesterase I. 113 enzyme units of phosphodiesterase I was admixed with 1 part water, then added to 7 parts paint. Each paint was mixed with a glass stirring rod and a paint mixer. Each film was immediately drawn onto polypropylene surfaces with a thickness of 8 mil. Cure time was 24 hours. Materials for assay were generated from the polypropylene surface as 1 cm², 2 cm² and 3 cm² free films.

The plate reader assay comprised: cutting free films into appropriate sized pieces and place them into microtubes, though blank samples have no paint film inside the microtube; adding 600 ul ddH₂O into each microtube; adding 750ul 200 mM TRIS into each microtube; and adding 150 uL of 14.5 mM Thymidine 5-monophosphate p-nitrophenyl ester sodium salt in ddH₂O into each microtube. Quality control and safety procedures were as described in Example 33, including use of a hood for material handling as appropriate.

Analysis included: taking out 100ul from each microtube at the appropriate time points, and reading the absorbance at 405 nm; and determining the initial rate slope by plotting absorbance vs. time to calculate phosphodiesterase I activity.

TABLE 100A
Phosphodiesterase I Sample absorbance at 405 nm

| Time (min) | Blank | | | 3 cm × 1 cm Control | | |
|---|---|---|---|---|---|---|
| 0 | 0.0432 | 0.0401 | 0.0438 | 0.0432 | 0.0401 | 0.0438 |
| 30 | 0.0385 | 0.0388 | 0.0384 | 0.0425 | 0.0441 | 0.0409 |
| 60 | 0.0412 | 0.0395 | 0.0391 | 0.0485 | 0.0402 | 0.0431 |
| 120 | 0.0408 | 0.0398 | 0.0394 | 0.0443 | 0.0408 | 0.0410 |
| 240 | 0.0410 | 0.0396 | 0.0442 | 0.0411 | 0.0421 | 0.0411 |
| 1200 | 0.0464 | 0.0411 | 0.0420 | 0.0433 | 0.0418 | 0.0416 |
| Slope (A/min) | 0.0000 | 0.0000 | 0.0000 | 0.0000 | 0.0000 | 0.0000 |

TABLE 100B
Phosphodiesterase I Sample absorbance at 405 nm

| Time (min) | 3 cm × 1 cm Enzyme | | | 2 cm × 1 cm Enzyme | | |
|---|---|---|---|---|---|---|
| 0 | 0.0432 | 0.0401 | 0.0438 | 0.0432 | 0.0401 | 0.0438 |
| 30 | 0.0582 | 0.0567 | 0.0598 | 0.0515 | 0.0486 | 0.0497 |
| 60 | 0.0807 | 0.0787 | 0.0822 | 0.0671 | 0.0628 | 0.0648 |
| 120 | 0.1459 | 0.1348 | 0.1424 | 0.1093 | 0.0997 | 0.1076 |
| 240 | 0.2720 | 0.2534 | 0.2663 | 0.2058 | 0.1854 | 0.1985 |
| 1200 | 0.6818 | 0.6674 | 0.6647 | 0.6234 | 0.5894 | 0.6073 |
| Slope (A/min) | 0.0010 | 0.0009 | 0.0010 | 0.0007 | 0.0006 | 0.0007 |

TABLE 100C
Phosphodiesterase I Sample absorbance at 405 nm

| Time (min) | 1 cm × 1 cm Enzyme | | |
|---|---|---|---|
| 0 | 0.0432 | 0.0401 | 0.0438 |
| 30 | 0.0459 | 0.0451 | 0.0455 |
| 60 | 0.0547 | 0.0509 | 0.0543 |
| 120 | 0.0800 | 0.0714 | 0.0793 |
| 240 | 0.1420 | 0.1151 | 0.1204 |
| 1200 | 0.4900 | 0.4191 | 0.4146 |
| Slope (A/min) | 0.0004 | 0.0003 | 0.0003 |

TABLE 101A
Phosphodiesterase I Sample absorbance Average at 405 nm

| Time | | 3 cm² | 3 cm² | 2 cm² | 1 cm² |
|---|---|---|---|---|---|
| (min) | Blank | Control | Phosphodiesterase I | Phosphodiesterase I | Phosphodiesterase I |
| 0 | 0.0424 | 0.0424 | 0.0424 | 0.0424 | 0.0424 |
| 30 | 0.0386 | 0.0425 | 0.0582 | 0.0499 | 0.0455 |
| 60 | 0.0399 | 0.0439 | 0.0805 | 0.0649 | 0.0533 |
| 120 | 0.0400 | 0.0420 | 0.1410 | 0.1055 | 0.0769 |
| 240 | 0.0416 | 0.0414 | 0.2639 | 0.1966 | 0.1258 |

TABLE 101B
Phosphodiesterase I Sample absorbance Deviation at 405 nm

| Time | | 3 cm² | 3 cm² | 2 cm² | 1 cm² |
|---|---|---|---|---|---|
| (min) | Blank | Control | Phosphodiesterase I | Phosphodiesterase I | Phosphodiesterase I |
| 0 | 0.0020 | 0.0020 | 0.0020 | 0.0020 | 0.0020 |
| 30 | 0.0002 | 0.0016 | 0.0016 | 0.0015 | 0.0004 |
| 60 | 0.0011 | 0.0042 | 0.0018 | 0.0022 | 0.0021 |
| 120 | 0.0007 | 0.0020 | 0.0057 | 0.0051 | 0.0048 |
| 240 | 0.0024 | 0.0006 | 0.0095 | 0.0103 | 0.0142 |

TABLE 102
Phosphodiesterase I Activity

| | Slope | U | U | U |
|---|---|---|---|---|
| Sample | (A/min) | (umol/min) | Average | Deviation |
| Blank | 0.0000 | −0.0004 | 0.00 | 0.00 |
| | 0.0000 | 0.0001 | | |
| | 0.0000 | 0.0024 | | |
| Control 3 cm² | 0.0000 | −0.0024 | 0.00 | 0.00 |
| | 0.0000 | 0.0005 | | |
| | 0.0000 | −0.0018 | | |
| Phosphodiesterase 3 cm² | 0.0010 | 0.2151 | 0.21 | 0.01 |
| | 0.0009 | 0.1987 | | |
| | 0.0010 | 0.2081 | | |
| Phosphodiesterase 2 cm² | 0.0007 | 0.1530 | 0.15 | 0.01 |
| | 0.0006 | 0.1362 | | |
| | 0.0007 | 0.1468 | | |
| Phosphodiesterase 1 cm² | 0.0004 | 0.0937 | 0.08 | 0.01 |
| | 0.0003 | 0.0703 | | |
| | 0.0003 | 0.0738 | | |

### Example 49

This Example describes identification and isolation of additional proteinaceous sequence(s) that may be used, such as a sequence possessing an antibiological activity.

Although a synthetically obtained peptidic agent (i.e., a peptide, polypeptide, a protein, an antifungal peptide) identified and produced as described herein (e.g., SEQ ID Nos. 1 to 47) may be used, it is also possible to employ suitable naturally produced peptidic agent (e.g., a microbe that produces a peptidic agent), as a component of a material formulation (e.g., an additive in a paint, a coating additive). A proteinaceous molecule, such as one possessing an antibiological activity, may be identified using an assay as described herein and/or the art. A number of such naturally occurring peptides are listed in the Table below, with reference citations often including activity assay(s) used in identification.

TABLE 103
Examples of Antibiological Peptides

| | | Seq. | | |
|---|---|---|---|---|
| Name | Source | ID | Activity | Reference |
| | Synthetic | 1 | Fungi | U.S. Pat. No. 5,885,782 |
| | Synthetic | 2 | Fungi | U.S. Pat. No. 5,885,782 |
| | Synthetic | 3 | Fungi | U.S. Pat. No. 5,885,782 |
| | Synthetic | 4 | Fungi | U.S. Pat. No. 5,885,782 |
| | Synthetic | 5 | Fungi | U.S. Pat. No. 5,885,782 |
| | Synthetic | 6 | Fungi | U.S. Pat. No. 5,885,782 |
| | Synthetic | 7 | Fungi | U.S. Pat. No. 5,885,782 |
| | Synthetic | 8 | Fungi | U.S. Pat. No. 5,885,782 |
| | Synthetic | 9 | Fungi | U.S. Pat. No. 5,885,782 |
| | Synthetic | 10 | Fungi | U.S. Pat. No. 5,885,782 |
| | Synthetic | 11 | Fungi | U.S. Pat. No. 5,885,782 |
| | Synthetic | 12 | Fungi | U.S. Pat. No. 5,885,782 |
| | Synthetic | 13 | Fungi | U.S. Pat. No. 5,885,782 |
| | Synthetic | 14 | Fungi | U.S. Pat. No. 5,885,782 |
| | Synthetic | 15 | Fungi | U.S. Pat. No. 5,885,782 |
| | Synthetic | 16 | Fungi | U.S. Pat. No. 5,885,782 |
| | Synthetic | 17 | Fungi | U.S. Pat. No. 5,885,782 |
| | Synthetic | 18 | Fungi | U.S. Pat. No. 5,885,782 |
| | Synthetic | 19 | Fungi | U.S. Pat. No. 5,885,782 |
| | Synthetic | 20 | Fungi | U.S. Pat. No. 5,885,782 |
| | Synthetic | 21 | Fungi | U.S. Pat. No. 5,885,782 |
| | Synthetic | 22 | Fungi | U.S. Pat. No. 5,885,782 |
| | Synthetic | 23 | Fungi | U.S. Pat. No. 5,885,782 |
| | Synthetic | 24 | Fungi | U.S. Pat. No. 5,885,782 |
| | Synthetic | 25 | Fungi | U.S. Pat. No. 5,885,782 |
| | Synthetic | 26 | Fungi | U.S. Pat. No. 5,885,782 |
| | Synthetic | 27 | Fungi | U.S. Pat. No. 5,885,782 |
| | Synthetic | 28 | Fungi | U.S. Pat. No. 5,885,782 |
| | Synthetic | 29 | Fungi | U.S. Pat. No. 5,885,782 |
| | Synthetic | 30 | Fungi | U.S. Pat. No. 5,885,782 |
| | Synthetic | 31 | Fungi | U.S. Pat. No. 5,885,782 |
| | Synthetic | 32 | Fungi | U.S. Pat. No. 5,885,782 |
| | Synthetic | 33 | Fungi | U.S. Pat. No. 5,885,782 |
| | Synthetic | 34 | Fungi | U.S. Pat. No. 5,885,782 |
| | Synthetic | 35 | Fungi | U.S. Pat. No. 5,885,782 |
| | Synthetic | 36 | Fungi | U.S. Pat. No. 5,885,782 |
| | Synthetic | 37 | Fungi | U.S. Pat. No. 5,885,782 |
| | Synthetic | 38 | Fungi | U.S. Pat. No. 5,885,782 |
| | Synthetic | 39 | Fungi | U.S. Pat. No. 5,885,782 |
| | Synthetic | 40 | Fungi | U.S. Pat. No. 5,885,782 |
| | Synthetic | 41 | Fungi | U.S. Pat. No. 5,885,782 |
| | Synthetic | 42 | Fungi | U.S. Pat. No. 5,885,782 |
| | Synthetic | 43 | Fungi | U.S. Pat. No. 5,885,782 |
| | Synthetic | 44 | Fungi | U.S. Pat. No. 5,885,782 |
| | Synthetic | 45 | Fungi | U.S. Pat. No. 5,885,782 |
| | Synthetic | 46 | Fungi | U.S. Pat. No. 5,885,782 |
| | Synthetic | 47 | Fungi | U.S. Pat. No. 5,885,782 |
| Tachystatin A | Horseshoe Crab | 48 | Gram+ & Gram−, | Fujitani (2002) |
| | | | Fungi | |
| Androctonin | Androctonus | 49 | Gram+ & Gram−, | Mandard (1999) |
| | Australis | | Fungi | |
| Tritrpticin | Synthetic | 50 | Gram+ & Gram−, | Schibli (1999) |
| | | | Fungi | |
| HNP-3 Defensin | Human | 51 | Gram+ & Gram−, | Hill (1991) |
| | | | Virus, Fungi | |
| Anti-fungal protein 1 | Phytolacca | 52 | Fungi | Gao (2001) |
| (pafp-s) | Americana | | | |
| Magainin 2 | Synthetic construct | 53 | Gram+ & Gram−, | Hara (2001) |
| | | | Fungi | |
| Indolicidin | Bos Taurus | 54 | Gram+ & Gram−, | Rozek (2000) |
| | | | Virus, Fungi | |
| Defensin heliomicin | Heliothis virescens | 55 | Fungi | Lamberty (2001) |
| Defensin heliomicin | Heliothis virescens | 56 | Gram+ & Gram−, | Lamberty (2001) |
| | | | Fungi | |
| Sativum defensin 1 | Seed of Pea | 57 | Fungi | Almeida (2002) |
| (psd1) | | | | |
| Gomesin | Synthetic | 58 | Gram+ & Gram−, | Mandard (2002) |
| | | | Fungi, Mammalian | |
| | | | cells | |
| Lactoferricin B | Bovine | 59 | Gram+ & Gram−, | Hwang (1998) |
| | | | Virus, Fungi, Cancer | |
| | | | cells | |
| PW2 | Synthetic | 60 | Fungi | Tinoco (2002) |
| Hepcidin 20 | Human | 61 | Fungi | Hunter (2002) |
| Hepcidin 25 | Human | 62 | Fungi | Hunter (2002) |
| AC-AMP2 | Amaranthus | 63 | Gram+, Fungi | Martins (1996) |
| | caudatus | | | |
| NK-Lysin | Sus scrofa | 64 | Gram+ & Gram−, | Liepinsh (1997) |
| | | | Fungi | |
| Magainin 2 | African clawed frog | 65 | Gram+ & Gram−, | Gesell (1997) |
| | | | Fungi, cancer cells | |
| Melittin B | Honey bee venom | 66 | Gram+ & Gram−, | Eisenberg |
| | | | Fungi, Mammalian | |
| | | | cells | |
| Thanatin | Podisus | 67 | Gram+ & Gram−, | Mandard (1998) |
| | maculiventris | | Fungi | |
| Antimicrobial | Common ice plant | 68 | Gram+ & Gram−, | Michalowski (1998) |
| peptide 1 | | | Fungi | |
| Melanotropin alpha | Bovine | 69 | Gram+, Fungi | Cutuli (2000) |
| (Alpha-MSH) | | | | |
| CORTICOSTATIN | Rabbit | 70 | Gram+ & Gram−, | Selsted (1988) |
| III (MCP-1) | | | Virus, Fungi | |
| CORTICOSTATIN | Rabbit | 71 | Gram+ & Gram−, | Selsted (1988) |
| III (MCP-1) | | | Virus, Fungi | |
| Cecropin B | Chinese oak silk | 72 | Gram+ & Gram−, | Qu (1982) |
| | moth | | Fungi | |
| Seminalplasmin | Bovine | 73 | Gram+ & Gram−, | Theil (1983) |
| | | | Fungi, Mammalian | |
| | | | cells | |
| NP-3A defensin | Rabbit | 74 | Gram+ & Gram−, | Zhu (1992) |
| | | | Virus, Fungi | |
| HNP-1 Defensin | Human | 75 | Gram+ & Gram−, | Zhang (1992) |
| | | | Virus, Fungi | |
| HNP-2 Defensin | Human | 76 | Gram+ & Gram−, | Selsted (1989) |
| | | | Virus, Fungi | |
| HNP-4 Defensin | Human | 77 | Gram+ & Gram−, | Wilde (1989) |
| | | | Fungi | |
| Histatin 5 | Human | 78 | Gram+ & Gram−, | Raj (1998) |
| | | | Fungi | |
| Histatin 3 | Human | 79 | Gram+ & Gram−, | Oppenheim (1988) |
| | | | Fungi | |
| Histatin 8 | | 80 | Gram+ & Gram−, | Yin (2003) |
| | | | Fungi | |
| Tracheal | Bovine | 81 | Gram+ & Gram−, | Zimmermann (1995) |
| antimicrobial peptide | | | Fungi | |
| AMP1 (MJ-AMP1) | Garden four-o'clock | 82 | Gram+, Fungi | Cammue (1992) |
| AMP2 (MJ-AMP2) | Garden four-o'clock | 83 | Gram+, Fungi | Cammue (1992) |
| MBP-1 | Maize | 84 | Gram+ & Gram−, | Duvick (1992) |
| | | | Fungi | |
| AFP2 | Rape | 85 | Fungi | Terras (1993) |
| AFP1 | Turnip | 86 | Fungi | Terras (1993) |
| AFP2 | Turnip | 87 | Fungi | Terras (1993) |
| ADENOREGULIN | Two coloured leaf | 88 | Gram+ & Gram−, | Mor (1994) |
| | frong | | Fungi | |
| Protegrin 2 | Pig | 89 | Gram+ & Gram−, | Kokryakov (1993) |
| | | | Virus, Fungi | |
| Protegrin 3 | Pig | 90 | Gram+ & Gram−, | Kokryakov (1993) |
| | | | Virus, Fungi | |
| Histatin 1 | Crab eating | 91 | Gram+ & Gram−, | Xu (1990) |
| | macaque | | Fungi | |
| Peptide PGQ | African clawed frog | 92 | Gram+ & Gram−, | Moore (1991) |
| | | | Fungi | |
| Ranalexin | Bull frog | 93 | Gram+ & Gram−, | Halverson (2000) |
| | | | Fungi | |
| GNCP-2 | Guinea pig | 94 | Gram+ & Gram−, | Nagaoka (1991) |
| | | | Virus, Fungi | |
| Protegrin 4 | Pig | 95 | Gram+ & Gram−, | Zhao (1994) |
| | | | Virus, Fungi | |
| Protegrin 5 | Pig | 96 | Gram+ & Gram−, | Zhao (1995) |
| | | | Virus, Fungi | |
| BMAP-27 | Bovine | 97 | Gram+ & Gram−, | Skerlavaj (1996) |
| | | | Fungi | |
| BMAP-28 | Bovine | 98 | Gram+ & Gram−, | Skerlavaj (1996) |
| | | | Fungi | |
| Buforin I | Asian toad | 99 | Gram+ & Gram−, | Park (1996) |
| | | | Fungi | |
| Buforin II | Asian toad | 100 | Gram+ & Gram−, | Yi (1996) |
| | | | Fungi | |
| BMAP-34 | Bovine | 101 | Gram+ & Gram−, | Scocchi (1997) |
| | | | Fungi | |
| Tricholongin | Trichoderma | 102 | Gram+ & Gram−, | Rebuffat (1991) |
| | longibrachiatum | | Fungi | |
| Dermaseptin 1 | Sauvage's leaf frog | 103 | Gram+ & Gram−, | Mor (1994) |
| | | | Fungi | |
| Pseudo-hevein | Para rubber tree | 104 | Fungi | Soedjanaatmadja |
| (Minor hevin) | | | | (1994) |
| Gaegurin-1 | Wrinkled frog | 105 | Gram+ & Gram−, | Park (1994) |
| | | | Fungi | |
| Skin peptide | Two-colored leaf frog | 106 | Gram+ & Gram−, | Mor (1994) |
| tyrosine-tyrosine | | | Fungi | |
| Penaeidin-1 | Penoeid shrimp | 107 | Gram+ & Gram−, | Destoumieux (2000) |
| | | | Fungi | |
| Neutrophil defensin | Golden hamster | 108 | Gram+, Fungi | Mak (1996) |
| 1 (HANP-1) | | | | |
| Neutrophil defensin | Golden hamster | 109 | Gram+, Fungi | Mak (1996) |
| 3 (HANP-3) | | | | |
| Misgurin | Oriental weatherfish | 110 | Gram+ & Gram−, | Park (1997) |
| | | | Fungi | |
| PN-AMP | Japenese morning | 111 | Gram+, Fungi | Koo (1998) |
| | glory | | | |
| Histone H2B-1 | Rainbow trout | 112 | Gram+ & Gram−, | Robinette (1998) |
| (HLP-1) (Fragment) | | | Fungi | |
| Histone H2b-3 | Rainbow trout | 113 | Fungi | Robinette (1998) |
| (HLP-3) (Fragment) | | | | |
| Neutrophil defensin | Rhesus macaque | 114 | Gram+ & Gram−, | Tang (1999) |
| 2 (RMAD-2) | | | Fungi | |
| Termicin | Pseudacanthotermes | 115 | Gram+, Fungi | Lamberty (2001) |
| | spiniger | | | |
| Spingerin | Pseudacanthotermes | 116 | Gram+ & Gram−, | Lamberty (2001) |
| | spiniger | | Fungi | |
| Aurein 1.1 | Southern bell frog | 117 | Gram+ & Gram−, | Rozek (2000) |
| | | | Fungi | |
| Ponericin G! | Ponerine ant | 118 | Gram+ & Gram−, | Orivel (2001) |
| | | | Fungi | |
| Brevinin-1BB | Rio Grande leopard | 119 | Gram+ & Gram−, | Goraya (2000) |
| | frog | | Fungi | |
| Ranalexin-1CB | Gree frog | 120 | Gram+ & Gram−, | Halverson (2000) |
| | | | Fungi | |
| Ranatuerin-2CA | Green frog | 121 | Gram+ & Gram−, | Halverson (2000) |
| | | | Fungi | |
| Ranatuerin-2CB | Green frog | 122 | Gram+ & Gram−, | Halverson (2000) |
| | | | Fungi | |
| Ginkbilobin | Ginkgo | 123 | Gram+ & Gram−, | Wang (2000) |
| | | | Virus, Fungi | |
| Alpha-basrubrin | Malabar spinach | 124 | Virus, Fungi | Wang (2001) |
| (Fragment) | | | | |
| Pseudin 1 | Paradoxical frog | 125 | Gram+ & Gram−, | Olson (2001) |
| | | | Fungi | |
| Parabutoporin | Scorpion | 126 | Gram+ & Gram−, | Moerman (2002) |
| | | | Fungi, Mammalian | |
| | | | cells | |
| Opistoporin 1 | African yellow leg | 127 | Gram+ & Gram−, | Moerman (2002) |
| | scorpion | | Fungi, Mammalian | |
| | | | cells | |
| Opistoporin 2 | African yellow leg | 128 | Gram+ & Gram−, | Moerman (2002) |
| | scorpion | | Fungi, Mammalian | |
| | | | cells | |
| Histone H2A | Rainbow trout | 129 | Gram+, Fungi | Fernandes (2002) |
| (fragment) | | | | |
| Dolabellanin B2 | Sea hare | 130 | Gram+ & Gram−, | Iijima (2002) |
| | | | Fungi | |
| Cecropin A | Nocutuid moth | 131 | Gram+ & Gram−, | Bulet (2002) |
| | | | Fungi | |
| HNP-5 Defensin | Human | 132 | Gram+ & Gram−, | Jones (1992) |
| | | | Fungi | |
| HNP-6 Defensin | Human | 133 | Gram+ & Gram−, | Jones (1993) |
| | | | Fungi | |
| Holotricin 3 | Holotrichia | 134 | Fungi | Lee (1995) |
| | diomphalia | | | |
| Lingual antimicrobial | Bovine | 135 | Gram+ & Gram−, | Schonwetter (1995) |
| peptide | | | Fungi | |
| RatNP-3 | Rat | 136 | Gram+ & Gram−, | Yount (1995) |
| | | | Virus, Fungi | |
| GNCP-1 | Guinea pig | 137 | Gram+ & Gram−, | Nagaoka (1993) |
| | | | Virus, Fungi | |
| Penaeidin-4a | Penoeid shrimp | 138 | Gram+ & Gram−, | Destoumieux (2000) |
| | | | Fungi | |
| Hexapeptide | Bovine | 139 | Gram+ & Gram−, | Vogle (2002) |
| | | | Virus, Fungi, Cancer | |
| | | | cells | |
| P-18 | | 140 | Gram+ & Gram−, | Lee (2002) |
| | | | Fungi, Cancer cells | |
| MUC7 20-Mer | Human | 141 | Gram+ & Gram−, | Bobek (2003) |
| | | | Fungi | |
| Nigrocin 2 | Rana nigromaculata | 142 | Gram+ & Gram−, | Park (2001) |
| | | | Fungi | |
| Nigrocin 1 | Rana nigromaculata | 143 | Gram+ & Gram−, | Park (2001) |
| | | | Fungi | |
| Lactoferrin (Lf) | | 144 | Fungi | Ueta (2001) |
| peptide 2 | | | | |
| Ib-AMP3 | Impatiens balsamina | 145 | Gram+, Fungi | Ravi (1997) |
| Ib-AMP4 | Impatiens balsamina | 146 | Gram+ Fungi | Ravi (1997) |
| Dhvar4 | Synthesis | 147 | Gram+ & Gram−, | Ruissen (2002) |
| | | | Fungi | |
| Dhvar5 | Synthesis | 148 | Gram+ & Gram−, | Ruissen (2002) |
| | | | Fungi | |
| | Synthetic | 149 | Fungi | U.S. App. 10/601,207 |
| | Synthetic | 150 | Fungi | U.S. App. 10/601,207 |
| | Synthetic | 151 | Fungi | U.S. App. 10/601,207 |
| | Synthetic | 152 | Fungi | U.S. App. 10/601,207 |
| | Synthetic | 153 | Fungi | U.S. App. 10/601,207 |
| | Synthetic | 154 | Fungi | U.S. App. 10/601,207 |
| | Synthetic | 155 | Fungi | U.S. App. 10/601,207 |
| | Synthetic | 156 | Fungi | U.S. App. 10/601,207 |
| | Synthetic | 157 | Fungi | U.S. App. 10/601,207 |
| | Synthetic | 158 | Fungi | U.S. App. 10/601,207 |
| | Synthetic | 159 | Fungi | U.S. App. 10/601,207 |
| | Synthetic | 160 | Fungi | U.S. App. 10/601,207 |
| | Synthetic | 161 | Fungi | U.S. App. 10/601,207 |
| | Synthetic | 162 | Fungi | U.S. App. 10/601,207 |
| | Synthetic | 163 | Fungi | U.S. App. 10/601,207 |
| | Synthetic | 164 | Fungi | U.S. App. 10/601,207 |
| | Synthetic | 165 | Fungi | U.S. App. 10/601,207 |
| | Synthetic | 166 | Fungi | U.S. App. 10/601,207 |
| | Synthetic | 167 | Fungi | U.S. App. 10/601,207 |
| | Synthetic | 168 | Fungi | U.S. App. 10/601,207 |
| | Synthetic | 169 | Fungi | U.S. App. 10/601,207 |
| | Synthetic | 170 | Fungi | U.S. App. 10/601,207 |
| | Synthetic | 171 | Fungi | U.S. App. 10/601,207 |
| | Synthetic | 172 | Fungi | U.S. App. 10/601,207 |
| | Synthetic | 173 | Fungi | U.S. App. 10/601,207 |
| | Synthetic | 174 | Fungi | U.S. App. 10/601,207 |
| | Synthetic | 175 | Fungi | U.S. App. 10/601,207 |
| | Synthetic | 176 | Fungi | U.S. App. 10/601,207 |
| | Synthetic | 177 | Fungi | U.S. App. 10/601,207 |
| | Synthetic | 178 | Fungi | U.S. App. 10/601,207 |
| | Synthetic | 179 | Fungi | U.S. App. 10/601,207 |
| | Synthetic | 180 | Fungi | U.S. App. 10/601,207 |
| | Synthetic | 181 | Fungi | U.S. App. 10/601,207 |
| | Synthetic | 182 | Fungi | U.S. App. 10/601,207 |
| | Synthetic | 183 | Fungi | U.S. App. 10/601,207 |
| | Synthetic | 184 | Fungi | U.S. App. 10/601,207 |
| | Synthetic | 185 | Fungi | U.S. App. 10/601,207 |
| | Synthetic | 186 | Fungi | U.S. App. 10/601,207 |
| | Synthetic | 187 | Fungi | U.S. App. 10/601,207 |
| | Synthetic | 188 | Fungi | U.S. App. 10/601,207 |
| | Synthetic | 189 | Fungi | U.S. App. 10/601,207 |
| | Synthetic | 190 | Fungi | U.S. App. 10/601,207 |
| | Synthetic | 191 | Fungi | U.S. App. 10/601,207 |
| | Synthetic | 192 | Fungi | U.S. App. 10/601,207 |
| | Synthetic | 193 | Fungi | U.S. App. 10/601,207 |
| | Synthetic | 194 | Fungi | U.S. App. 10/601,207 |
| | Synthetic | 195 | Fungi | U.S. App. 10/601,207 |
| | Synthetic | 196 | Fungi | U.S. App. 10/601,207 |
| | Synthetic | 197 | Gram+ & Gram−, | U.S. App. 10/601,207 |
| | | | Fungi | |
| | Synthetic | 198 | Gram+ & Gram−, | U.S. App. 10/601,207 |
| | | | Fungi | |
| | Synthetic | 199 | Gram+ & Gram−, | U.S. App. 10/601,207 |
| | | | Fungi | |

A natural source may provide additional sequences to be used for a material formulation (e.g., a coating additive). In some embodiments, the use of a natural antifungal products isolated in commercial quantity from a microorganism may use a large-scale cell culture (e.g., culture of an antifungal agent-producing microorganism) for the production and purification of the peptidic (e.g., an antifungal) product. In some aspects, the cultural isolate responsible for the production of the endogenously produced proteinaceous molecule (e.g., an antifungal peptidic agent) may be batch-cultured. In some facets, a purification technique and/or strategy, such as those described herein and/or in the art, may be used purify the active product to a reasonable (e.g., desired) level of homogeneity. However, in some aspects, a naturally derived peptidic agent (e.g., an antifungal agent) may co-purify with an unwanted microbial byproducts, especially a byproduct which may be undesirably toxic. Purification of an endogenously produced proteinaceous composition may result in a racemized mixture wherein one or more stereoisomer(s) are active, and/or wherein a disulfide linkage may occur (e.g., a disulfide linkage between peptide monomers). When a desirable naturally occurring proteinaceous molecule (e.g., an antifungal protein, an antifungal polypeptide, an antifungal peptide) may be isolated, for example, and the amino acid sequences at least partially identified, synthesis of the native molecule, or portions thereof, may use a specific disulfide bond formation, a high histidine requirement, and so forth. Of course, once a proteinaceous molecule is sequence is identified, and/or a nucleotide sequence for a proteinaceous molecule is isolated, it then may be recombinantly expressed using techniques described herein and/or in the art.

### Example 50

This Example describes assay protocols for evaluating antifungal coatings. It is contemplated that such assays may be adapted to also assay other types of material formulations comprising various biomolecular composition(s) and activity against other types of biological cells.

A suitable assay protocol for evaluating a coating comprising an antifungal agent which may be applied in assaying an antifungal peptide is described by the American Society for Testing and Materials (ASTM) in D-5590-94 (“Standard Test Method for Determining the Resistance of Paint Films and Related Coatings to Fungal Defacement by Accelerated Four-Week Agar Plate Assay”). The assay method may be modified as indicated below, and generally comprises: preparing a set of four 1×10 cm aluminum coupons approximately 1/32 in thick will be prepared as follows: (1) blank Al coupon; (2) Al coupon coated with an aqueous solution of a peptide produced and identified as described herein, and allowed to dry; (3) Al coupon coated on both sides with a base paint composition, allowed to dry, and then the paint film will be coated with a like amount of the same test peptide solution as applied to coupon 2; and (4) Al coupon painted with a paint mixture comprising the same base paint composition as for coupon 3 and a like amount of the peptide, as for coupons 2 and 3. Duplicate or triplicate sets of these specimens may be prepared. Optionally, a conventional biocide may be included as a positive control. The base paint composition may be any suitable water-based latex paint, without biocides, which is available from a number of commercial suppliers.

Each of the specimens from (a) will be placed on a bed of nutrient agar and uniformly innoculated with a fungal suspension. An example test organism comprises a Fusarium oxysporum. The fungal suspension may be applied by atomizer or by pipet, however a thin layer of nutrient agar mixed with the fungal innoculum may be used. The specimens are incubated at about 28° C. under 85 to 90% relative humidity for 4 weeks. Fungal growth on each specimen is often rated weekly as follows: None=0; traces of growth (<10% coverage)=1; light growth (10-30%)=2; moderate growth (30-60%)=3; and heavy growth (60% to complete coverage)=4.

Another suitable assay protocol for testing the antifungal properties of a coating or paint film containing an antifungal peptide is described by the ASTM in D-5590-94 (“Standard Test Method for Resistance to Growth of Mold on the Surface of Interior Coatings in an Environmental Chamber”). The testing protocol generally includes:

Preparation of the Coated Surface. Duplicate or triplicate sets of approximately ½ in. thick, 3×4 in. untreated wooden or gypsum board panels will be prepared as follows: (1) blank panel; (2) coated with an aqueous solution of a peptide produced and identified as described herein, and allowed to dry; (3) coated on both sides with a base paint composition, allowed to dry, and then the paint film is coated with a like amount of the same test peptide solution as applied to panel 2; and (4) painted with a paint mixture containing the same base paint composition as for panel 3 and a like amount of the peptide, as for panels 2 and 3. Optionally, a conventional biocide may be included as a positive control.

Contamination: The panels will be randomly arranged and suspended in an environmental cabinet above moist soil that has been inoculated with the desired fungus, usually a Fusarium oxysporum. Enough free space is provided to allow free circulation of air and avoiding contact between the panels and the walls of the cabinet.

Incubation: The panels will be incubated for two weeks at 30.5-33.5° C. and 95-98% humidity.

Scoring: A set of panels (test, control, and, optionally, a positive control) will be removed for analysis at intervals, usually weekly. The mold growth on the specimen panels may be rated as described above.

Alternatively, one or more equivalent testing protocols may be employed, and field assays of coating compositions containing laboratory-identified antifungal peptide(s) and/or candidate peptide(s) may be carried out in accordance with conventional methods of the art.

### Example 51

This Example describes assay protocols for evaluating a latex paint comprising an antifungal peptidic agent. It is contemplated that such assays may be adapted to also assay other types of material formulations comprising various biomolecular composition(s) and activity against other types of biological cells.

Both the interior latex (Olympic Premium, flat, ultra white, 72001) and acrylic paints (Sherwin Williams DTM, primer/finish, white, B66W1; 136-1500) appeared to be toxic to both Fusarium and Aspergillus. Therefore, eight individual wells (48-well microtito plate) of each paint type were extracted on a daily basis with 1 ml of phosphate buffer for 5 days (1-4 & 6) and then allowed the plates were allowed to dry before running the assay. Each well contained 16 ul of respective paint.

Extract testing: The extract from two wells each of the two paints for each day was tested for toxicity by mixing the extract 1:1 with 2× medium and inoculating with spores (10⁴) of Aspergillus or Fusarium. The extracts had no affect on growth of either test fungus.

Well testing: The extracted and non-extracted wells for each of the paints were tested with a range of inoculum levels in growth medium using the two different fungi. For Fusarium the range was 10¹-10⁴ and for Aspergillus 10²-10⁵.

Well Testing of Acrylic Paint Plates: Both Fusarium and Aspergillus grew in all extracted wells at all inoculum levels. Only Aspergillus grew in non-extracted wells at the 10⁵ level and not at lower levels indicative of an inherent biocidal capability.

Well Testing of Latex Paint Plates: Fusarium grew in the extracted wells only at the 10⁴ inoculum level but not at 10¹-10³. Aspergillus grew in all extracted wells showing an inoculum level effect. No growth was observed for either Fusarium or Aspergillus in non-extracted wells.

Conclusion: Extraction of the toxic factor(s) found in both paints was possible. However, it appeared that it may be less extractable from the latex paint.

Evaluation of peptide activity in presence of acrylic and latex paints: It was established that it was possible to extract both acrylic and latex paints dried in a 48-well format to make them non-toxic to the test microorganisms-Fusarium and Aspergillus. Using that information an experiment was designed to determine the effect the paint has on peptide activity against two test organisms.

Experimental design: Coat 48-well plastic plates with 16 μl of acrylic or latex paint. Dry for two days under hood. Extract designated wells with 1-ml phosphate buffer changing the buffer on a daily basis for 7 days. Control wells were not extracted to confirm paint toxicity. Add 20 μl of peptide series in duplicate to designated dry paint coated wells. Peptide, SEQ ID No. 41, series were added in a two-fold dilution series to wells and allowed to dry. The concentration of peptide added ranged from 200 μg/20 μl to 1.5 μg/20p1.

Inoculated paint-coated plates as follows: Extracted control wells received 180 μl of medium+20 μl of spore suspension (10⁴spores/20 μl of medium). Inoculum was either Fusarium or Aspergillus in each case. Non-extracted control wells received 180 μl of medium+20 μl of spore suspension (10⁴spores/20 μl of medium). Extract wells with dried peptide series received 180 μl of medium+20 μl of spore suspension (10⁴ spores/20 μl of medium). In duplicate. Extract wells that did not have dried peptide series received 160 μl of medium+20 μl of spore suspension (10⁴120 μl of medium)+20 μl peptide series as above. In duplicate. Plates were observed for growth over a 5-day period.

Growth and peptide controls: Use sterile non-paint coated 48 well plastic plates. Growth control wells for each test fungus received 180 μl of medium+20 μl of spore suspension (10⁴ spores/20 μl of medium). Peptide activity controls received 160 μl of medium+20 μl of spore suspension (10⁴ spores/20 μl of medium)+20 μl peptide series as above. Peptide series were added in a two-fold dilution series to wells and range from 200 μg/20 μl to 1.5 μg/20 μl. Therefore, the range of peptide tested was 200 μg/200 μl or 1.0 μg/p1 (1000 μg/ml) to 0.0075 μg/μl (7.5 μg/ml). Uninoculated medium served as blank for absorbance readings taken at 24, 48, 72, 96 and 120 h.

Results: Unextracted wells containing either latex or acrylic paint inhibited growth of both Fusarium and Aspergillus. Extracted wells containing either latex or acrylic paint allowed growth of both Fusarium and Aspergillus. The calculated MIC for Fusarium in peptide activity control experiments was 15.62 μg/ml. For Aspergillus the calculated MIC was 61.4 μg/ml.

For extracted acrylic-coated plates the following results were obtained. Controls as stated in above. For Fusarium with dried peptide, inhibition was seen at 1000 and 500 μg/ml after 5 days. Spores exposed to liquid peptide added to dry paint wells were inhibited at 1000, 500 and 250 μg/ml after 4 days, and 1000 and 500 μg/ml after 5 days. For Aspergillus with dried peptide, inhibition was seen at 1000 μg/ml after 5 days. Spores exposed to liquid peptide added to dry paint wells were inhibited at 1000 and 500 μg/ml after 5 days.

For extracted latex-coated plates the following results were obtained. Controls as stated above. For Fusarium with dried peptide, inhibition was seen at 1000 μg/ml after 5 days. Spores exposed to liquid peptide added to dry paint wells were inhibited at 1000 μg/ml after 5 days. For Aspergillus with dried peptide, inhibition was seen at 1000 μg/ml after 5 days. Spores exposed to liquid peptide added to dry paint wells were inhibited at 1000 μg/ml after 5 days.

### Example 52

This Example describes combinations of an antibiological proteinaceous composition and an antibiological agent such as a standard preservative.

A material formulation (e.g., a paint composition) comprising one or more conventional antibiological substance(s) (e.g., a preservative, an antimicrobial agent, an antifungal substance) may be modified by addition of one or more of the antibiological proteinaceous composition(s) (e.g., an antifungal peptide) described herein. For example, combining a non-peptidic antibiological agent (e.g., antifungal agent) with one or more antibiological proteinaceous molecule(s) (e.g., an antifungal peptide) may provide antifungal activity over and above that seen with either the proteinaceouos or the non-peptidic agent alone. The expected additive inhibitory activity of the combination is calculated by summing the inhibition levels of each component alone. The combination is then assayed on the assay organism to derive an observed additive inhibition. If the observed additive inhibition is greater than that of the expected additive inhibition, synergy is exhibited. For example, a synergistic combination of a proteinaceous molecule (e.g., an aliquot of a peptide library, a peptide) comprising at least one antibiological proteinaceous molecule (e.g., an antifungal peptide) occurs when two or more cell (e.g., fungal cell) growth-inhibitory substances distinct from the proetinaceous molecule are observed to be more inhibitory to the growth of an assay organism than the sum of the inhibitory activities of the individual components alone.

An example of an assay method for determining additive or synergistic combinations comprises first creating a synthetic peptide combinatorial library. Each aliquot of the library represents an equimolar mixture of peptides in which at least the two C-terminal amino acid residues are known. Using the testing methods described in one or more of U.S. Pat. No. 6,020,312, U.S. Pat. No. 5,885,782, and U.S. Pat. No. 5,602,097 it is possible to determine for each such aliquot of the synthetic peptide combinatorial library, a precisely calculated concentration at which it will inhibit an assayed fungus in a coating. Next, the aliquot of the synthetic peptide combinatorial library is mixed with at least one non-peptide antifungal compound to create an assay mixture. As with the peptide component of the mixture, the baseline ability of the non-peptide antifungal substance to inhibit the test fungus is determined initially. Next, the assay fungus is contacted with the assay mixture, and the inhibition of growth of the assay organism is measured as compared to at least one untreated control. More controls are desirable, such as a control for each individual component of the mixture. Similarly, where there are more than two components being tested, the number of controls to be used must be increased in a manner in the art of growth inhibition assays. From the separate assay results for the peptidic and the non-peptidic agent(s) the expected additive effect on inhibition of growth is determined using standard techniques. After the growth inhibition assay(s) are complete for the combination of peptidic and the non-peptidic agent(s), the actual or observed effect on the inhibition of growth is determined. The expected additive effect and the observed effect are then compared to determine whether a synergistic inhibition of growth of the test fungus has occurred. The methods used to detect synergy may utilize non-peptide antimicrobial agents in combination with the inhibitory peptides described herein.

### Example 53

This Example describes coating a surface to inhibit fungus infestation and growth.

When anchorage, food and moisture are available, a cell such as a microorganism (e.g., a fungus) are able to survive where temperatures permit. Susceptible surfaces may include a porous material such as a stone, a brick, a wall board (e.g., a sheetrock) and/or a ceiling tile; a semi-porous material, including a concrete, an unglazed tile, a stucco, a grout, a painted surface, a roofing tile, a shingle, a painted and/or a treated wood and/or a textile; or a combination thereof. Any type of indoor object, outdoor object, structure and/or material that may be capable of providing anchorage, food and moisture to fungal cells is potentially vulnerable to infestation with mold, mildew or other fungus. Moisture generally appears due to condensation on surfaces that are at or below the dew point for a given relative humidity.

To inhibit or prevent fungus infestation and growth, one or more antifungal peptidic agents described herein (e.g., approximately 250-1000 mg/L of the hexapeptide of SEQ ID No. 41), may be dissolved or suspended in water and applied by simply brushing and/or spraying the solution onto a pre-painted surface such as an exterior wall that may be susceptible to mold infestation. Conventional techniques for applying or transferring a coating material to a surface in the art are suitable for applying the antifungal peptide composition. The selected peptide(s) have activity for inhibiting or preventing the growth of one or more target fungi. The applied peptide solution is then dried on the painted surface, usually by allowing it to dry under ambient conditions. If desired, drying can be facilitated with a stream of warm, dry air. Optionally, the application procedure may be repeated one or more times to increase the amount of antifungal peptide that is deposited per unit area of the surface. As a result of the treatment, when the treated surface is subsequently subjected to the target mold organisms or spores and growth promoting conditions comprising humidity above about typical indoor ambient humidity, presence of nutrients, and temperature above about typical indoor ambient temperature and not exceeding about 38° C., the ability of the surface to resistance fungal infestation and growth is enhanced compared to its pre-painted condition before application of the antifungal peptide.

A simple spray-coated surface may provide sufficient durability for certain applications such as surfaces that are exposed to weathering, though longer-term protection may be provided against adhesion and growth of mold by mixing one or more of the antifungal peptides with a base paint or other coating composition, which may be any suitable, commercially available product in the art. The base composition may be free of chemicals and other additives that are toxic to humans or animals, and/or that fail to comply with applicable environmental safety rules or guidelines. The typical components, additives and properties of conventional paints and coating materials, and film-forming techniques, of the art, described herein, and/or described in U.S. patent application Ser. No. 10/655,345 filed Sep. 4, 2003, U.S. patent application Ser. No. 10/792,516 filed Mar. 3, 2004, and U.S. patent application Ser. No. 10/884,355 filed Jul. 2, 2004, may be used.

If additional, long-term protection against growth and adhesion of a mold, a mildew and/or a fungus is desired, the paint or other coating composition may include a barrier material that resists moisture penetration and also prevents or deters penetration and adhesion of the microorganisms and the airborne contaminants which serve as food for the growing organisms. Some typical water repellent components are an acrylic, a siliconate, a metal-stearate, a silane, a siloxane and/or a paraffinic wax. The user may take additional steps to deter mold infestation include avoiding moisture from water damage, excessive humidity, water leaks, condensation, water infiltration and flooding, and taking reasonable steps to avoid buildup of organic matter on the treated surface.

### Example 54

This Example describes a method of treating a fungus-infested surface.

In situations where existing fungal growth is present, the mold colonies and/or spores may be removed and/or substantially eliminated before application of one of an antifungal coating, it is expected that in some situations an antifungal compositions may be applied to existing mold infected surfaces. In this case, the composition, comprising one or more antifungal peptides, may inhibit, arrest the growth of, or substantially eradicate the mold. Early detection and treatment may be used in order to minimize the associated discoloration or other deterioration of the underlying surface due to mold growth. The treatment procedure may comprise applying one or more coats of an antifungal peptide solution and/or a coating composition (e.g., a paint) as described herein.

### Example 55

This Example relates to the use of a polymeric material such as a plastic (e.g., a thermoplatic, a thermoset). It is contemplated that a biomolecular composition (e.g., an enzyme) may also be incorporated into a polymeric material. A polymeric material may comprise a plurality of polymers (“polymer blends”), an ionomer, a thermoplastic polymer, a thermoset polymer, or an elastomer. A thermoplastic comprises a thermoplastic polymer, while a thermoset plastic comprises a thermosetting polymer. A thermoplastic polymeric material may, for example, comprise a biodegradable polymer, a cellulosic polymer, a fluoropolymer, a polyether, a polyamide, a polyacrylonitrile, a polyamide-imide, a polyarylate, a polybenzimidazole, a polybutylene, a polycarbonate, a thermoplastic polyester, a polyetherimide, a polyethylene, a polyimide, a polyketone, an acrylic, a polymethylpentene, a polyphenylene oxide, a polyarylene sulphide, a polypropylene, a polyurethane, a polystyrene, a polysulfone resin, a polyterpene, a polyvinyl acetal, a polyvinyl acetate, a thermoplastic vinyl ester, a polyvinyl ether, a polyvinyl carbazole, a polyvinyl chloride, a polyvinylidene chloride, a polyimidazopyrrolone, a polyacrolein, a polyvinylpyridine, a polyvinylamide, a polyurea, a polyquinoxaline, or a combination thereof. A thermoplastic polymer may comprise an environmentally degradable polymers (e.g., a biodegradable polymer), a natural polymer, a photodegradable polymer, a synthetic biodegradable polymer (e.g., a poly(alkylene oxalate)-s-, a polyamino acid, a pseudo-polyamino acid, a polyanhydride, a polycaprolactone, a polycyanoacrylate, a polydioxanone, a polyglycolide, poly(hexamethylene-co-trans-1,4-cyclohexane dimethylene oxalate), a polyhydroxybutyrate, a polyhydroxyvalerate, a polylactide, a poly(ortho ester), a poly(p-dioxanone), a polyphosphazene, a poly(propylene fumarate), a polyvinyl alcohol), a biological degradable polymer (e.g., a collagen, a fibrinogen/fibrin, a gelatin, a polysaccharide), a cellulosic polymer (e.g., cellulose acetate, a cellulose acetate butyrate, a cellulose acetate propionate, a cellulose methylcellulose, a methylcellulose, a cellulosehydroxyethyl, an ethylcellulose, a hydroxypropylcellulose), a fluoropolymer, an ethylene chlorotrifluoroethylene, an ethylene tetrafluoroethylene, a fluoridated ethylene propylene, a polyvinylidene fluoride, a polychlorotrifluoroethylene, a polytetrafluoroethylene, a polyvinyl fluoride), a polyoxymethylene, a polyamide, an aromatic polyamide, a polyacrylonitrile, a polyamide-imide, a polyarylate, a polybenzimidazole, a polybutylene, a polycarbonate, a polyester (e.g., a liquid crystal polyester polycarbonate, a polybutylene terephthalate, a polycyclohexylenedimethylene terephthalate, a poly(ethylene terephthalate)), a polyetherimide, polyethylene (e.g., a very low-density polyethylene, a low-density polyethylene, a linear low-density polyethylene, a medium-density polyethylene, a high-density polyethylene, an ultrahigh molecular weight polyethylene, a chlorinated polyethylene, a chlorosulfonated polyethylene, a phosphorylated polyethylene, an ethylene-acrylic acid copolymer, an ethylene-methyl acrylate copolymer, an ethylene-ethyl acrylate copolymer, an ethylene-n-butyl acrylate copolymer, an ethylene-vinyl acetate copolymer, an ethylene-vinyl alcohol copolymer), a polyimide, a polyketone, a poly(methylmethacrylate), a polymethylpentene, a polyphenylene oxide, a polyphenol sulfide, a polyphthalamide, a polypropylene, a polyurethane, a polystyrene (e.g., styrene-acrylonitrile copolymer, a styrene-butadiene copolymer, an acrylonitrile butadiene styrene terpolymer, an acrylonitrile-chlorinated polyethylene-styrene terpolymer, an acrylic styrene acrylonitrile terpolymer), a polysulfone resin (e.g., a polysulfone, a polyaryl sulfone, a polyether sulfone), a polyvinyl chloride (e.g., a chlorinated polyvinyl chloride), a polyvinylidene chloride, or a combination thereof. A thermoset polymeric material may comprise, for example, an alkyd resin, an allyl resin, an amino resin, a bismaleimide resin, a cyanate ester resin, an epoxy resin, a furane resin, a phenolic resin, a thermosetting polyester resin, a polyimide resin, a polyurethane resin, a silicone resin, a vinyl ester resin, a casein, or a combination thereof. Polymeric materials often comprise an additive, such as a filler, a plasticizer, a lubricant, a flame retarder, a colorant, a blowing agent, an anti-aging additive, a cross-linking agent, etc. or a combination thereof. Polymeric materials and methods of preparation of preparing a polymeric material and assays for a polymeric material's properties have been described, for example, “Handbook of Plastics, Elastomers, & Composites Fourth Edition” (Harper, C. A. Ed.) McGraw-Hill Companies, Inc, New York, 2002; and Tadmor, Z. and Costas, G. G. “Principles of Polymer Processing Second Edition,” John Wiley & Sons, Inc. Hoboken, New Jersey, 2006.

### REFERENCES

“ASTM Book of Standards, Volume 06.01, Paint—Tests for Chemical, Physical, and Optical Properties; Appearance” (2002) ASTM International, West Conshohocken, Pa., U.S.A.
“ASTM Book of Standards, Volume 06.02, Paint—Products and Applications; Protective Coatings; Pipeline coatings” (2002) ASTM International, West Conshohocken, Pa., U.S.A.
“ASTM Book of Standards, Volume 06.03, Paint—Pigments, Drying Oils, Polymers, Resins, Naval Stores, Cellulosic Esters, and Ink Vehicles” (2002) ASTM International, West Conshohocken, Pa., U.S.A.
“ASTM Book of Standards, Volume 06.04, Paint—Solvents; Aromatic Hydrocarbons” (2002) ASTM International, West Conshohocken, Pa., U.S.A.
“Concise Encyclopedia of Polymer Science and Engineering,” (Kroschwitz, Jacqueline, I) John Wiley & Sons, Inc. Hoboken, N.J., 1990.
“Directed Enzyme Evolution: Screening and Selection Methods (Methods in Molecular Biology) (Arnold, F. H. and Georgiou, G) Humana Press, Totowa, N.J., 2003.
“Handbook of Plastics, Elastomers, & Composites Fourth Edition” (Harper, C. A. Ed.) McGraw-Hill Companies, Inc, New York, 2002.
“Paint and Coating Testing Manual, Fourteenth Edition of the Gardner-Sward Handbook” (Koleske, J. V., Ed.) (1995) American Society for Testing and Materials, Philadelphia, Pa., U.S.A.
“Paint and Surface Coatings, Theory and Practice, Second Edition” (Lambourne, R. and Strivens, T. A., Eds.) (1999) Woodhead Publishing Ltd., Cambridge, England.
“Paints, Coatings and Solvents, Second, Completely Revised Edition” (Stoye, D. and Freitag, W., Eds.) (1998) Wiley-Vch, New York, U.S.A.
“Reactive Modifiers for Polymers,” (Al-Malaika, S., Ed.) Chapman & Hall, London, UK, 1997.
“Silanes and other Coupling Agents,” (Mittal, K. L., Ed.) Koninklijke Wohrmann B. V. The Netherlands, 1992.
“ASTM Book of Standards, Volume 06.01, Paint—Tests for Chemical, Physical, and Optical Properties; Appearance” (2002) ASTM International, West Conshohocken.
Abdel-Fattah, Y, R., and Gaballa A A. Microbiol. Res. 163(1):13-20, 2008.
Abe, H., Doi, Y., Aoki, H., Akehata, T., Hori, Y., Yamaguchi, A., Macromolecules 28:7630-7637, 1995.
Abe, H., Doi, Y., Aoki, H., Akehata, T., Macromolecules 31:1791-1797, 1998.
Adamitsch, B. F. et al., Lett Appl Microbiol 36:227-229, 2003.
Ahmed, K. et al. J Biosci Bioeng 95:27-34, 2003.
Ahn, J. M. et al., Chem Commun (Camb). (4):364-365, 2004.
Ahn, J. O. et al., Appl Microbiol Biotechnol. 64(6):833-839, 2004.
Alam, M. et al., Biochemistry. 41(21):6679-6687, 2002.
Alam, M. et al., J Lipid Res. 47(2):375-383, 2006.
Albaret, A. et al., Prot. Struct. Funct. Genet. 28:543-555, 1997.
Alberts, B. et al. Essential Cell Biology. 2nd ed. Garland Science, Taylor & Francis Group. New York, 2004.
Albizo, J. M. and White, W. E. “The Hydrolysis of GD and VX by Acetone Dried Preparations of Cured and Plasmid-Containing Pseudomonas Diminuta” Chemical Research, Development & Engineering Center Scientific conference on Chemical Defense Research, November 18-21, pp. 643-649, 1986.
Allouch, J., et al. J. Biol. Chem. 278:47171-47180, 2003.
Almeida, et al., J. Mol. Biol. 315(4); 749-57, 2002.
Alquati, C. et al., Eur J. Biochem. 269(13):3321-3328, 2002.
Altmann, F. et al. Biochem. Biophys. Res. Commun. 136:329-335, 1986.
Aminlari, M. et al. J Sci Food Agric 85:2617-2624, 2005.
Andreana, P. R., Xie, W., Wang, P. G., Biocatalysis in Polymer Science ACS Symposium Series 840:188, 2002.
Andreopoulos, F. M. et al., Biotech. Bioeng. 65(5):579-588, 1999.
Andrews, D. L. et al., Biochem J. 252(1):199-206, 1988.
Andrzejewska, E., Progress in Polymer Science 26:605-665, 2001.
Archer D. B. et al. BiolTechnol. 8:741-745, 1990.
Argentine Patent No. AR026954B1
Armugam, A. et al., Toxicon. 35(1):27-37 1997.
Ash, M. and Ash, I. “Handbook of Paint and Coating Raw Materials, Second Edition” (1996) Ashgate Publishing Company, Brookfield, Vt., U.S.A.
Ashani, et al., Biochem. Pharm. 55:159-168, 1998.
Assaf, N. A., and Dick, W. A., Biotechniques, 15:1010-1015, 1993.
Astrachan, L. Biochim. Biophys. Acta 296:79-88, 1973.
Augusteyn, R. C. et al., Biochim. Biophys. Acta 171:128-137, 1969.
Australian Patent AU2003220057
Azzoni, A. R. et al., Biotech. Bioeng. 80(3):268-276, 2002.
Baba, T. and Schneewind, O. EMBO J. 15:4789-4797, 1996
Baldridge, G. D. et al, Current Microbiology 51:233-238, 2005
Baptista, R. P. et al., J Biotechnol. 102(3):241-249, 2003.
Barbeyron, T. et al. J. Biol. Chem. 275:35499-35505, 2000.
Barras, D. R. and Stone, B. A. Biochim. Biophys. Acta 191:329-341, 1969a.
Barras, D. R. and Stone, B. A. Biochim. Biophys. Acta 191; 342-353, 1969b.
Bauer, C.-A. Eur. J. Biochem. 105:565-570, 1980.
Baxter, G. D. et al., Insect Biochem. and Molec. Bio., 28:581-589 (1998).
Baxter, G. D. et al., Insect Biochem. and Molecular Bio., 32:815-820 (2002).
Beger, B. and Faber, K. J. Chem. Soc., Chem. Commun., 1198, 1991.
Belardinelli, M. et al., Ann Trop Med. Parasitol. 99(7):673-682, 2005.
Ben, Ali Y. et al., Protein Expr Purif. 51(2):162-169, 2007.
Bennet, H., Industrial Waxes Volume II Compounded Waxes and Technology, Chemical Publishing Co, New York, 1975.
Benning, M. M. et al., Biochem. 33:15001-15007, 1994.
Benning, M. M. et al., Biochem. 34:7973-7978, 1995.
Benning, M. M. et al., J. Biol. Chem. 275:30556-30560, 2000.
Benschop, H. P. and De Jong, L. P. A. Acc. Chem. Res. 21:368-374, 1988.
Benschop, H. P. et al., Toxic. and Applied Pharm. 72:61-74, 1984.
Berg, J. M., Tymoczko, J. L., Stryer, L., Biochemistry 5^{th} Ed. Freeman Company. New York 2001.
Bigey, F. et al., Yeast. 20(3):233-248, 2003.
Billecke, S. S. et al., Chemico-Biological Interactions 119-120, 251-256, 1999.
Bishwabhusan, S., Anupam, B., Hongyong, F., Wei, G., Gross, R. A., Biomacromolecules 7:1042-1048, 2006.
Blackburn, S. A. et al. Microbiology 144:73-82, 1998.
Blade, C. C. F. et al. Proc. R. Soc. Lond. B: Biol. Sci. 167:378-388, 1967a.
Blake, C. C. F. et al. Proc. R. Soc. Lond. B: Biol. Sci. 167:365-377, 1967b.
Blaner, W. S., et al., Biochim. Biophys. Acta 794:419-427, 1984.
Blaner, W. S., et al., J. Biol. Chem. 262:53-58, 1987.
Blow, D. M. Acc. Chem. Res. 9:145-152, 1976.
Bobek, et al., Antimicrob Agents Chemother 47(2); 643-52, 2003.
Boer, E. et al., Yeast. 22(7):523-535, 2005.
Booth, G., et al., Industrial Crops and Products, 25(3): 257-265, 2007.
Bosmann, H. B. Biochim. Biophys. Acta. 276:180-191, 1972.
Breinig, F. et al., Appl Environ Microbiol. 72(11):7140-7147, 2006.
Brinkmann, U. et al., Gene 85:109-114, 1989.
Brocca, S. et al., Protein Sci. 12(10):2312-2319, 2003.
Brockerhoff, H. and Jensen, R. G. “Lipolytic Enzymes.” Academic Press, Inc., New York, N.Y., 1974.
Broedl, U. C. et al., Circ Res. 94(12):1554-1561, 2004.
Broomfield, C. A., et al., Chemico-Biochem. Interactions., 119-120:413-418 (1999).
Browder, H. P., et al., Biochem. Biophys. Res. Commun., 19, 383, 1965.
Brunel, L. et al., J Biotechnol. 111(1):41-50, 2004.
Brunke, S., and Hube B. et al., Microbiology. 152(Pt 2):547-554, 2006.
Bugg, C. E. et al., Sci. Am., 269:92-98, 1993.
Bulet, et al., submitted to SWISS-PROT data bank; 2002.
Cablo, Ed., Handbook of Coatings Additives, pp. 177-224, 1987. Calado C R, et al., J Biosci BioEng. 93(4):354-359, 2002.
Calado, C. R. et al., J Biotechnol. 109(1-2):147-158, 2004.
Calado, C. R. et al., J Biosci BioEng. 96(2):141-148, 2003.
Calandra, G. B., and Cole, R. M., Infect. Immun., 28:1033-1037, 1980.
Caldwell, S. R. and Raushel, F. M., Appl. Biochem Biotech 31:59-74, 1991b.
Caldwell, S. R. and Raushel, F. M., Biochem. 30:744-7450, 1991c.
Caldwell, S. R. and Raushel, F. M., Biotech. Bioeng. 37:103-109, 1991a.
Cammue, et al., J. Biol. Chem. 267; 2228-33, 1992.
Campbell, P. M. et al., Biochem. Molec. Biol. 28:139-150, 1998.
Campbell, Paint & Coating Testing Manual, 14^{th} Ed. of Gardner-Sward Handbook, Ch. 54, pp. 654-61, 1995.
Canfield, R. E., J. Biol. Chem., 238:2698-2707, 1963.
Chae, M. Y. et al., Bioorg. Med. Chem. Lett. 4:1473-1478, 1994.
Chang, S. W. et al., Appl Microbiol Biotechno1.67(2):215-224, 2005.
Chang, S. W. et al., J Agric Food Chem. 54(16):5831-5838, 2006^{A}.
Chang, S. W. et al., J Agric Food Chem.54(3):815-822, 2006^{C}.
Chang, S. W. et al., J Mol Microbiol Biotechnol. 11(1-2):28-40, 2006^{B}.
Chaplin M. Enzyme Technology. Cambridge University Press. UK 1990.
Chatterjee, S, and Ghosh, N. J. Biol. Chem. 264:12554-12561, 1989.
Chatterjee, S., Russell, A. J. Biotechnology and Bioengineering 40:1069-1077, 1992.
Chen, T. et al., Biomacromolecules 2:456-462, 2001.
Chen, W. and Mulchandani, A. Tibtech 16:71-76, 1998.
Cheng, T.-C. et al., Appl. Environ. Microbiol. 62(5):1636-1641, 1996.
Cheng, T.-C. et al., Applied and Environ. Microbio. 59(9):3138-3140, 1993.
Cheng, T.-C. et al., Chemico-Biological Interactions 119-120:455-462, 1999.
Cheng, T.-C. et al., J. Ind. Microbiol. 18:49-55, 1997.
Chen-Goodspeed, M. et al., Biochemistry 40:1332-1339, 2001 b.
Chen-Goodspeed, M. et al., Biochemistry, 40:1325-1331, 2001a.
Chesters, C. G. C. and Bull, A. T. Biochem. J. 86:31-38, 1963.
Chiang, T. et al., Bull. Env. Contam. Toxicol. 34:809-814, 1985.
Chien, S., et al. Biochem. Biophys. Res. Commun. 76:317-323, 1977.
Cho, A. R. et al., FEMS Microbiol Lett. 186(2):235-238, 2000.
Cho, C. M. et al., Applied and Enviro. Microbio., 2026-2030, 2002.
Chohnan et al. FEMS Microbiol Lett 213:13-20, 2002.
Chohnan, S. et al., FEMS Microbiol Lett 213:13-20, 2002.
Choi, G. S. et al., Protein Expr Purif. 29(1):85-93, 2003.
Claudianos, C. et al., Insect Biochem. and Molecular Bio. 29:675-686, 1999.
Cohen, A. A. and Shatzmiller, S. E., J. Mol. Graph. 11:166-173, 1993.
Cohen, J. A. and Warring, M. G. Biochim. Biophys. Acta. 26:29-39, 1957.
Coller, B. S. et al., J. Biol Chem. 268:20741-20743, 1993.
Columbia patent application USMA:042C0
Combes, D. et al., J. Mol. Biol. 300:727-742, 2000.
Contreras, J. A. et al., Protein Expr Purif. 12(1):93-99, 1998.
Cotes, K. et al., Biochem J. 408(3):417-427, 2007.
Coulthard, M. G. et al., Infect Immun. 64(5):1510-1515, 1996.
Cousin, X. et al., J. Biol. Chem. 271(25):15099-15108, 1996.
Creighton, T. E. Proteins: Structure and Molecular Properties, W. H. Freeman & Co., San Francisco, pp 79-86, 1983.
Cunningham, L. W. and Manners, D. J. Biochem. J. 80:42 P-43P, 1961.
Cutuli, et al., J Leukoc Biol. 27(2); 233-39, 2000.
Daniels, A. et al., J. Appl. Biomater. 1:57, 1990.
Datta, P. K., et al. Can. J. Biochem. Physiol. 41:697-705, 1963
Dave, K. I. et al., Appl. Microbiol. Biotechnol. 41:352-358, 1994b.
Dave, K. I. et al., Biotechnol. Appl. Biochem. 19:271-284, 1994a.
Dave, K. I. et al., Chemical-Biological Interactions 87:55-68, 1993.
Davies, R. C., et al., Biochim., Biophys. Acta, 178:294-305, 1969.
De Simone, G. et al., J Mol. Biol. 303(5):761-771, 2000.
Dean, P. M., BioEssays, 16:683-687, 1994.
DeFrank, J. J. and Cheng, T.-C., J. Bacteriol 173:1938-1943, 1991.
DeFrank, J. J. et al., Chem.-Biol. Interact. 87:141-148, 1993.
Desai, U. A. et al., Protein Expr Purif25(1):195-202, 2002.
Destoumieux, et al., Cell. Mol. Life. Sci. 57; 1260-71, 2000.
Detry, J. et al., Appl Microbiol Biotechnol. 72(6):1107-1116, 2006.
Dharmsthiti, S. et al., J Gen Appl Microbiol. 44(2):139-145, 1998.
Di Fulvio, M. et al., J Mol. Biol. 367(3):814-824, 2007.
Di Lorenzo, M. et al., Appl Environ Microbiol. 71(12):8974-8977, 2005.
Diaz-Alejo, N. et al., Chem.-Biol. Interact. 108(3):187-196, 1998.
DiPersio, L. P. et al., Protein Expr Purif. 3(2):114-120, 1992.
diSioudi, B. D. et al., Chemico-Biological Interactions 119-120:211-223, 1999b.
diSioudi, B. et al., Biochemistry 38:2866-2872, 1999a.
Dixon M., Webb E. C., Enzymes, 2^{nd} Ed. Academic Press Inc. New York 1964.
Dixon, N. E. et al. Science 191:1144-1150, 1976.
Dodgson, K. S. et al., Biochem. J. 64: 216-221, 1956.
Donarski, W. J. et al., Biochemistry 28:4650-4655, 1989.
Downes, C. P. and Michell, R. H. Biochem. J. 198:133-140, 1981
Downs, D. et al., Biochemistry. 33(26):7979-7985, 1994.
Drevon, G. F. and Russell, A. J., Biomacromolecules 1:571-576, 2000.
Drevon, G. F. et al., Biomacromolecules 2:664-671, 2001.
Drevon, G. F. et al., Biotechnology and Bioengineering 79(7):785-794, 2002.
D'Silva, S. et al., J Lipid Res.48(11):2478-2484, 2007.
Duckworth, M. and Turvey, J. R. Biochem. J. 113:687-692, 1969.
Duda, A., Kowalski, A., Penczek, S., Uyama, H., Kobayashi, S., Macromolecules 35:4266-4270, 2002.
Dugi, K. A. et al., J Lipid Res. 38(9):1822-1832, 1997.
Dumas, D. P. et al., Biotech. Appl. Biochem. 11:235-243, 1989a.
Dumas, D. P. et al., Arch. Biochem. Biophys. 277:155-159, 1990.
Dumas, D. P. et al., Experientia, 46:729-731, 1990.
Dumas, D. P. et al., The Journal of Bio. Chem. 264(33):19659-19665, 1989b.
Dunning, H. R., Pressure Sensitive Adhesives-Formulations and Technology, 2^{nd} Ed., Noyes Data Corporation, New Jersey, 1977.
Dunning, H. R., Pressure Sensitive Adhesives-Formulations and Technology, 2^{nd} Ed., Noyes Data Corporation, New Jersey, 1977.
Durban, M. A. et al., Appl Microbiol Biotechnol. 74(3):634-639, 2007.
Dusek, K., Progress in Polymer Science 25:1215-1260, 2000.
Duvick, et al., J. Biol. Chem. 267; 18814-20, 1992.
Duysen, E. G., J. Pharm. Exp. Ther. 302:751-758, 2002.
Edwards, R and Owen, W. J., Planta 175:99-106, 1998.
Efremenko, E. N. et al., J. Biochem. Biophys Methods 51:195-201, 2002.
Egelrud, T. and Olivecrona, T. Biochim. Biophys. Acta 306:115-127, 1973.
Eisenberg, et al.
Ejima, K. et al., J Biosci BioEng. 98(6):445-451, 2004.
Elashvili, I. and Defrank, J. J. “Phosphonate transporter mutation enhances the utilization of diisopropylphosphate (DIPP) and diisopropyl fluorophosphates (DFP) in Escherichia coliK-12. Proceedings of 1996 US Army ERDEC Scientific conference on chemical Defense Research, U.S. Army ERDEC, Aberdeen Proving Ground, Aberdeen, Md.
Elashvili, I. et al., Appl Environ Microbiol 64(7):2601-2608, 1998.
Elend, C. et al., J Biotechnol. 130(4):370-377, 2007.
Elliott, B. W. and Cohen, C. J. Biol. Chem. 261:11259-11265, 1986.
Ellman, G. L. et al., Biochem Pharmacol 7:88-95, 1961.
Endo, F. et al., J. Biol. Chem. 264(8):4476-4481, 1989.
Engelberg, I. and Kohn, J. Biomaterials 12:292, 1991.
Enzyme nomenclature. Recommendations 1992, Eur. J. Biochem. 223:1-5, 1994.
Enzyme nomenclature. Recommendations 1992, Eur. J. Biochem. 232:1-6, 1995.
Enzyme nomenclature. Recommendations 1992, Eur. J. Biochem. 237:1-5, 1996.
Enzyme nomenclature. Recommendations 1992, Eur. J. Biochem. 250:1-6, 1997.
Enzyme nomenclature. Recommendations 1992, Eur. J. Biochem. 264:610-650, 1999.
Ertel, S, and Kohn, J. J. Biomed. Mater. Res. 28:919, 1994.
Esposito, R. and A. M. Fletcher. Arch. Biochem. Biophys. 93:369-376, 1961.
Eydoux, C. et al., J Lipid Res. 48(7):1539-1549, 2007.
Ezaki, T. and Suzuki, S., J. Clin. Microbiol., 16:844-846, 1982.
Faber, K. “Biotransformations in Organic Chemistry, a Textbook, Third Edition.” Springer-verlag Berlin Heidelberg, 1997.
Fan, J. et al., J Biol Chem. 276(43):40071-4009, 2001.
Ferlinz, K. et al., J Biol Chem. 276(38):35352-35360, 2001.
Fernandes, et al., Biochem. J. 368; 611-20, 2002.
Fernández, L. et al., Protein Expr Purif. 49(2):256-264, 2006.
Fickers, P. et al., J Biotechnol. 115(4):379-386, 2005.
Fiedler, et al., J. Chem. Technol. Biotechnol., 32:271-280, 1982.
Fiedler, F. Eur. J. Biochem. 163:303-312, 1987.
Fiedler, H. P., et al. J. Chem. Technol. Biotechnol. 32:271-280, 1982.
Fischer, E. H. and Stein, E. A. Cleavage of O- and S-glycosidic bonds (survey), in Boyer, P. D., Lardy, H. and Myrbäck, K. (Eds.), The Enzymes, 2nd edn., vol. 4, Academic Press, New York, pp. 301-312, 1960.
Fletcher, T. S. et al., Biochemistry 26:3081-3086, 1987.
Flick, E. W. “Handbook of Paint Raw Materials, Second Edition” (1989) Noyes Data Corporation/Noyes Publications, Park Ridge, N.J., U.S.A.
Flick, E. W., “Prepaint Specialties and Surface Tolerant Coatings,” Noyes Publications (1991).
Flick, E. W., “Textile Finishing Chemicals: An Industrial Guide,” Noyes Publications (1996).
Flick, E. W., Adhesive and Sealant Compound Formulations, 2^{nd} Ed., Noyes Publications, New Jersey 1984.
Flick, E. W., Adhesive and Sealant Compound Formulations, 2^{nd} Ed., Noyes Publications, New Jersey, 1984.
Flick, E. W., Construction and Structural Adhesives and Sealants, Noyes Publications, New Jersey, 1988.
Flick, E. W., Construction and Structural Adhesives and Sealants, Noyes Publications, New Jersey, 1988.
Flick, E. W., Handbook of Paint Raw Materials, Noyes Data Corporation/Noyes Publications, Park Ridge, N.J., U.S.A., 1982.
Flick, E. W., Handbook of Paint Raw Materials, Second Edition, Noyes Data Corporation/Noyes Publications, Park Ridge, N.J., U.S.A., 1989.
Flick, E. W., Industrial Surfactants, Noyes Publications, New Jersey, 1988.
Flick, E. W., Textile Finishing Chemicals: An Industrial Guide, Noyes Publications, 1990.
Flick, E., Contemporary Industrial Coatings-Environmentally Safe Formulations, Noyes Publications, New Jersey, 1985.
Flick, E., Engineering Resins—An Industrial Guide, Noyes Publications, New Jersey, 1988.
Flick, E., Handbook of Raw Adhesives, 2^{nd} Ed., Noyes Publications, New Jersey, 1989.
Flick, E., Handbook of Raw Adhesives, Noyes Publications, New Jersey, 1982.
Flick, E., Water-Soluble Resins—An Industrial Guide, Noyes Publications, New Jersey, 1986.
Flick, Handbook of Paint Raw Materials, Second Edition, 263-85, 879-998, 1989.
Fliss, I., et al., Biotechniques, 11:453-457, 1991.
Fogorasi, M. and Heine, E., Central European Journal of Chemistry, 4(4):786-797, 2006).
Folk, J. E. Methods Enzymol. 19:109-112, 1970.
Fox, M. A., Acc. Chem. Res. 16:314-321, 1983.
Fox, T. G., Bulletin of the American Physics Society, 1:123, 1956.
Fredrikson, G., et al., J. Biol. Chem. 256:6311-6320, 1981.
Fujikawa, R. et al., Lipids. 40(9):901-908, 2005.
Fujita, T. et al., Biochim. Biophys. Acta 1308 (1):49-57, 1996.
Fujitani, et al., J. Biol. Chem. 277; 23651, 2002.
Fukui, S, and Tanaka, A. Adv. Biochem. Eng. Biotechnol. 29:33, 1984.
Furka, A., et al. Int. J. Pept. Protein Res. 37:487, 1991.
Furka, et al., Int. J. Pept. Protein Res., 37:487, 1991.
Gaberlein, S. et al., Analyst 125:2274-2279, 2000.
Gaberlein, S. et al., Appl Microbiol Biotechnol 54:652-658, 2000a.
Gaeng, S. et al Appl Environ Microbiol 66:2951-2958, 2000.
Gallo, M. A. and Lawryk, N.J. (1991) Organic phosphorous pesticides. In: The Handbook of Pesticide Toxicology (Eds. Hayes, W. J. Jr. and Laws, E. R) Academic Press, San Diego, Calif. pp. 920-925.
Gao, et al., Biochemistry 40 (37); 10973-78, 2001.
Gao, J. and Simon, M. J Invest Dermatol. 124(6):1259-1266, 2005.
Gao, J. et al., J. Agric. Food Chem. 48:614-6120, 2000.
Garcia-Lepe, R., et al., Lett. Appl. Microbiol. 25:127-130, 1997.
Garden, J. M. et al., Comp. Biochem. Physiol. 52C:95-98, 1975.
Gargouri, Y. et al., Eur J. Biochem., 180(2):367-371, 1989.
Gaskin, D. J. et al., Biotechnol BioEng. 73(6):433-441, 2001.
Gerard, J. F., ed., Fillers and Filled Polymenrs-Macromolecular Symposia 169, Wiley-VCH, Verlag, 2001.
Gesell, et al., J. Biomol. NMR 9; 127, 1997.
Gheshlaghi, R. et al. Biotechnol Bioeng 90:754-760, 2005.
Ghosh, S. Physiol Genomics. 2(1):1-8, 2000.
Ghuysen, J.-M. et al. Biochemistry 8:213-222, 1969.
Gillette, M. L., “Using Acid-Base Indicators to Visually Estimate the pH of Solutions,” Chemical Education Resources, Inc. (1985).
Glascock, C. B. and Weickert, M. J. Gene, 223(1-2):221-231, 1998.
Gopal, S. et al., Biochem. and Biophys. Research Comm. 279:516-519, 2000.
Goraya, et al., Eur. J. Biochem. 267; 894-900, 2000.
Gordon, R. K. et al., Chemico-Biological Interactions 119-120:463-470, 1999.
Grauso, M. et al., FEBS Letter 424:279-284, 1998.
Greene, T. W. and Wuts, P. G. M. Second Edition, pp. 309-315, John Wiley & Sons, Inc., USA, 1991.
Greten, H. et al., Biochim. Biophys. Acta 210:39-45, 1970.
Grimsley, J. K. “Enhancement of OPH production” Final report, U.S. Army Project DAAG-55-97-C-0005, 1997.
Grimsley, J. K. et al., Biochemistry 36(47):14366-14374, 1997.
Grimsley, J. K. et al., Biotechnology Intl. 2:235-242, 1999.
Gubitz, G. M., Paulo, A. C., Current Opinion in Biotechnology 14:577-582, 2003.
Guichard, et al., PNAS USA, 91:9765-69, 1994.
Gustaysson, M. et al., Protein Eng. 14(9):711-715, 2001.
Gutfreund, H. and Sturtevant, J. M. Biochem J. 63:656-661, 1956.
Gyamerah, M. et al. Appl Microbiol Biotechnol 60:403-407, 2002.
Haack, M. B. et al., Biotechnol BioEng. 96(5):904-913, 2007.
Haacke, G., Andrawes, F. F., Campbell, B. H., Journal of Coatings Technology 68:57-62, 1996.
Hagen, F. S., et al., Biochemistry 30:8415-8423, 1991.
Hall, E. et al., Am J Physiol Gastrointest Liver Physiol. 281(1):G293-301, 2001.
Halverson, et al., Peptides 21; 469-76, 2000.
Han, S. J. et al., Biochimie. 85(5):501-510, 2003.
Hancock, R. E. W. and Scott, M. G. PNAS 97(16): 8856-8861, 2000
Hannig, G. and Makrides, S.C. TIBTECH 16:54-60, 1998.
Hara, et al., Biopolymers 58(4); 437-46, 2001.
Harel, M. et al., J. Am. Chem. Soc. 118:2340-2346, 1996.
Harel, M. et al., Proc Natl Acad Sci USA 89(22):10827-10831, 1992.
Harper, Charles A. and Petrie, Edward M. “Plastic Materials and Processes A Concise Encyclopedia,” John Wiley & Sons, Inc. Hoboken, N.J., 2003.
Harper, L. et al., Appl. Env. Micro. 54:2586-2589, 1988.
Hartleib, J. and Ruterjans, H Biochim et Biophys Acta 1546:312-324, 2001b.
Hartleib, J. and Ruterjans, Prot. Expression and Purification 21:210-219, 2001a.
Hartleib, J. et al., Biochem J 353:579-589, 2001.
Hartshorn, S. R., ed., Structural Adhesives-Chemistry and Technology, Plenum Press, New York, 1986.
Hassett, C. et al., Biochemistry 30:10141-10149, 1991.
Hatfield, R. and Nevins, D. J. Carbohydr. Res. 148:265-278, 1986
Havens, P. L. and Rase, H. F. Ind. Eng. Chem. Res. 32:2254-2258, 1993.
Helmsing, P. J. Biophys. Acta 178:519-533, 1969.
Henderson, L. A., Svirkin, Y. Y., Gross, R. A., Kaplan, D. L., Swift, G., Macromolecules 29:7759-7766, 1996.
Herbold, D. R. and Glaser, L. J. Biol. Chem. 250:1676-1682, 1975.
Hill, C. M. et al., Bioorganic Chemistry, 29:27-35, 2001.
Hill, C. M. et al., Bioorganic Medicinal Chemistry Letters 10:1285-1288, 2000.
Hill, et al., Science 251; 1481-85, 1991.
Hiramatsu, T. et al., J Biol Chem. 278(49):49438-49447, 2003.
Hiraoka, M. et al., J Biol Chem. 277(12):10090-10099, 2002.
Hirayama, 0., et al., Biochim. Biophys. Acta 384:127-137, 1975.
Holden, G., ed., et. al., Thermoplastic Elastomers, 2^{nd} Ed., Hanser Publishers, Verlag, 1996.
Holler, H., et al., Biochem., 14:2377-2385, 1975.
Holtje, J.-V. et al. J. Bacteriol 124:1067-1076, 1975.
Hong, M. S. et al., Bioremediation Journal 2(2):145-157, 1998.
Hong, S. et al., Biochim Biophys Acta. 1735(3):222-229, 2005.
Hong, S.-B. and Raushel, F. M Chemico-Bio. Interact. 119-120:225-234, 1999b.
Hong, S.-B. and Raushel, F. M. Biochem. 35:10904-10912, 1996.
Hong, S.-B. and Raushel, F. M. Biochem. 38:1159-1165, 1999a.
Horgan, D. J. et al., Biochemistry 8:2000-2006, 1969.
Horne, I. et al., Appl. Environ. Microbiol. 68(7):3371-3376, 2002.
Hoskin, F. C. G. “An organophosphorus detoxifying enzyme unique to squid.” In: Squid as Experimental Animals (Eds. Gilbert, D. L., Adelman W. J. Jr. and Arnold, J. M.), pp. 469-480. Plenum Press, New York, 1990.
Hoskin, F. C. G. and Roush, A. H., Science 215:1255-1257, 1982.
Hoskin, F. C. G. et al., Biochemical Pharmacology 46(7):1223-1227, 1993.
Hoskin, F. C. G. et al., Fundam. Appl. Toxicol. 4:5165-5172, 1984.
Hoskin, F. C. G. et al., Biochemical Pharmacology 34(12):2069-2072, 1985.
Hoskin, F. C. G. et al., Biochemical Pharmacology 49(5):711-715, 1995.
Hoskin, F. C. G. et al., Chemico-Biological Interactions 119-120:399-404, 1999.
Hoskin, F. C. G. et al., Chemico-Biological Interactions 119-120:439-444, 1999.
Houghten, BioTechniques, 13:412, 1992.
Houghten, Nature, 354:84, 1991.
Hruby, V. J., Biopolymers, 33:1073-1082, 1993.
Huang, A. H. C. et al., Plant Physiol. 61:339-341, 1978.
Huber, R. and Bode, W. Acc. Chem. Res. 11:114-122, 1978.
Huddleston, S. et al., Biochem Biophys Res Commun. 216(2):495-500, 1995.
Hung, S.-C. and Liao, J. C., Appl. Biochem. Biotechnol. 56(1):37-47, 1996.
Hunter, et al., J. Biol. Chem. 277; 35797, 2002.
Hwang, et al., Biochemistry 37; 4288, 1998.
Ibrahim, H. R. et al. J Biol Chem 269:5059-5063, 1994.
Wei, A. et al., Appl Microbiol Biotechnol. 58(3):322-329, 2002.
Iijima, et al., Dev. Comp. Immunol. 0; 2002.
Ikeda, S. et al., J Biosci BioEng. 98(5):366-373, 2004.
Ikemura, H., et al., J. Biol. Chem. 262:7859-7864, 1987.
In “Advances in Protein Chemistry, Volume 45 Lipoproteins, Apolipoproteins, and Lipases.” (Anfinsen, C. B., Edsall, J. T., Richards, Frederic, R. M., Eisenberg, D. S., and Schumaker, V. N. Eds.) Academic Press, Inc., San Diego, Calif., 1994.
In “Chemical Warfare Agents: Toxicity at Low Levels” (Satu M. Somani and James A. Romano, Jr., Eds.) CRC Press, Boca Raton, 2001. Chapter 1, Health Effects of Low-Level Exposure to Nerve Agents, p 2.
In “Chemical Warfare Agents: Toxicity at Low Levels” (Satu M. Somani and James A. Romano, Jr., Eds.) CRC Press, Boca Raton, 2001. Chapter 14, Emergency Response to a Chemical Warfare Agent Incident: Domestic Preparedness, First Response, and Public Health Considerations, p 414.
In “Chemical Warfare Agents: Toxicity at Low Levels” (Satu M. Somani and James A. Romano, Jr., Eds.) CRC Press, Boca Raton, 2001. Chapter 2, Toxicokinetics of Nerve Agents, pp 26-29.
In “Colour Index International” 3^{rd} Ed. Pigment and Solvent Dyes, Society of Dyers and Colourists American Association of Textile Chemists and Colorists, 1997.
In “Colour Index International” 3^{rd} Ed. Society of Dyers and Colourists American Association of Textile Chemists and Colorists, 1971.
In “Current Protocols in Cell Biology” (Morgan, K. Ed.) John Wiley & Sons, 2002.
In “Current Protocols in Cytometry” (Robinson, J. P. Ed.) John Wiley & Sons, 2002.
In “Current Protocols in Immunology” (Coico, R. Ed.) John Wiley & Sons, 2002.
In “Current Protocols in Molecular Biology” (Chanda, V. B. Ed.) John Wiley & Sons, 2002.
In “Current Protocols in Molecular Biology, Chapter 16, Protein Expression,” John Wiley & Sons, Inc, pp. 1-4, 1987, 2002.
In “Current Protocols in Nucleic Acid Chemistry” (Harkins, E. W. Ed.) John Wiley & Sons, 2002.
In “Current Protocols in Pharmacology” (Taylor, G. Ed.) John Wiley & Sons, 2002.
In “Current Protocols in Protein Science” (Taylor, G. Ed.) John Wiley & Sons, 2002.
In “Emulsion Polymer Technologies,” the April, 2002 edition, the Paint Research Association.
In “Engineering of/with Lipases” (F. Xavier Malcata., Ed.) Kluwer Academic Publishers, Dordrecht, The Netherlands, 1996.
In “Industrial water-based paint formulations” by Ernest W. Flick, Park Ridge, N. J. Noyes, (1988), xvi, 277; p. 25.
In “Lipases and Phospholipases in Drug Development from Biochemistry to Molecular Pharmacology.” (Müller, G. and Petry, S. Eds.) WiLEY-VCH, Weinheim, Germany 2004.
In “Lipases their Structure, Biochemistry and Application” (Paul Woolley and Steffen B. Peterson, Eds.) Cambridge University Press, Great Britain, 1994.
In “Lipases” (Borgstrom, B. and Brockman, H. L., Eds) Elsevier Science Publishers B. V., Amsterdam, The Netherlands, 1984.
In “Molecular Cloning” (Sambrook, J., and Russell, D. W., Eds.) 3rd Edition, Cold Spring Harbor, N.Y.: Cold Spring Harbor Laboratory Press, 2001.
In “Organic Coatings: Science and Technology” 2^{nd} edition, by Zeno W. Wicks Jr., Frank N. Jones, S. Peter Pappas, Publisher: Wiley-Interscience (John Wiley & Sons, Inc. 605 Third Avenue, New York, N.Y.) Table 31.1 Exterior White House Paint, p. 562.
In “Paint and surface coatings: Theory and Practice” 2^{nd} Edition (Lambourne, R. and Strivens, T. A. William, Eds) Andrew Publishing, Woodhead Publishing Ltd, Abington Hall, Abington, Cambridge CB1 6AH, England, 1999.
In “Paints, Coatings and Solvents” 2^{nd} Edition (Stoye, D. and Freitag, W., Eds) Wiley-Vch, New York, 1998.
In “Paints, Coatings and Solvents, Second, Completely Revised Edition,” (Stoye, D. and Freitag, W., Eds.) pp. 6, 12-19, 127, 165, 288-290, 1998.]
Inohue, M. et al. J. Plant Physiol. 154:334-340, 1999.
International patent publication: WO 01/72911 A1.
International Patent WO 01/44380 A2
International Patent WO 03/076709
Ishida, T. et al., Genomics. 83(1):24-33, 2004.
Ishiguro, S. et al., Plant Cell. 13(10):2191-2209, 2001.
Ishii, T. et al., Biochim. Biophys. Acta 1308(1):15-16, 1996.
Isono, et al., J. Chem. Technol. Biotechnol., 32:271-80, 1979.
Isono, K. and S. Suzuki. Heterocycles 13:333-351, 1979.
IUBM B (1992) Enzyme Nomenclature: Recommendations (1992) of the Nomenclature Committee of the International Union of Biochemistry and Molecular Biology. (NC-ICBMB and Edwin C. Webb Eds.) Academic Press, San Diego, Calif.
Jahns, T., et al. Can. J. Microbiol. 43:1111-1117, 1997.
Jam, M. et al. Biochem. J. 385:703-713, 2005.
Jekel, P. A., et al., Anal. Biochem. 134:347-354, 1983.
Jenkins, C. M. et al., J Invest Dermatol. 124(6):1259-1266, 2005.
Jiang, Z. et al., Appl Microbiol Biotechnol. 70(3):327-332, 2006.
Jiang, Z. et al., Mol. Biotechnol. 31(2):95-101, 2005.
Jiang, Z. B. et al., Protein Expr Purif. 56(1):35-39, 2007.
Johnson, E. N. et al., AATCC Review, 12:40-44, 2006.
Johnson, K., Antistatic Compositions for Textiles and Plastics, Noyes Data Corporation, New Jersey, 1976.
Johnson, K., Antistatic Compositions for Textiles and Plastics, Noyes Data Corporation, New Jersey, 1976.
Jolles, P., Angewandte Chemie, International Edition, 8:227-239, 1969.
Jones, et al., FEBS Lett. 315; 187-92, 1993.
Jones, et al., J. Biol. Chem. 367; 23216-25, 1992.
Josse, D. et al., Chemico-Biological Interactions 119-120:71-78, 1999.
Josse, D. et al., J. Appl. Toxicol. 21:S7-S11, 2001.
Kaieda, M. et al., Appl Microbiol Biotechnol. 65(3):301-305, 2004.
Kakugawa, K. et al., Biosci Biotechnol Biochem. 66(6):1328-1336, 2002.
Kakugawa, S. et al., Appl Microbiol Biotechnol. 74(3):585-591, 2007.
Kanamori, T. et al., J. Bacteriol. 186:2532-2539, 2004.
Kanehisa, M. and Goto, S, Nucleic Acids Res. 28:27-30, 2000.
Kanehisa, M. et al. Nucleic Acids Res. 34:D354-357, 2006.
Kanehisa, M. et al. Nucleic Acids Res. 36:D480-D484, 2008.
Kaneva, I. et al., Biotechnol. Prog. 14:275-278, 1998.
Kanfer, J. N., et al., J. Biol. Chem. 241:1081-1084, 1966.
Kapteyn, J. C., et al., Glycobiology., 6, 337-345, 1996.
Karlsson, M. et al., J Biol Chem. 272(43):27218-27223, 1997.
Karlsson, M. et al., Protein Expr Purif. 18(3):286-292, 2000.
Karube, I. et al., Appl. Microbiol. Biotechnol. 21:270-272, 1985.
Kawai, E. et al., J Biosci BioEng. 91(4):409-415, 2001.
Kenten, R. H. and Mann, P. J. G. Biochem. J. 57:347-348, 1954.
Kepka, C. et al., J Chromatogr A. 1075(1-2):33-41, 2005.
Kim, C. et al., Biotechnol Bioeng 65:108-113, 1999.
Kim, H. K. et al., Biosci Biotechnol Biochem. 62(1):66-71, 1998.
Kim, J. T. et al., Appl Microbiol Biotechnol. 74(4):820-828, 2007.
Kim, J.-W. et al., Biotechnol. Prog. 18:429-436, 2002.
Kim, M. H. et al., Biosci Biotechnol Biochem. 64(2):280-286, 2000.
Kim, S, and Lee, S. B. Biosci Biotechnol Biochem. 68(11):2289-2298, 2004.
Kim, S. D., Klein, A., Sperling, L. H., Macromolecules 33:8334-8343, 2000.
Kim, Y. Mol. Cells. 18(1):40-45, 2004.
King, Paint & Coating Testing Manual, 14^{th} Ed. of Gardner-Sward Handbook, Ch. 29, pp. 261-67, 1995.
Kitaura, S. et al., J. Biochem. 129(3):397-402, 2001.
Kobayashi et al., Chemical Communication pp. 4227-4229, 2006.
Kobayashi, R et al, J. Ferment Technol. 59:21-26, 1981.
Kobayashi, S., Uyama H., Kimura, S., Chemical Reviews 101(12), 3793-3818, 2001.
Kobayashi, S., Uyama, H., Takamoto, T., Biomacromolecules 1:3-5, 2000.
Kobayashi, T. et al., Jpn J Med Sci Biol. 49(3):103-112, 1996.
Koepke, J. et al., Acta. Cryst. D58:1757-1759, 2002.
Koide, N. and Muramatsu, T. J. Biol. Chem. 249:4897-4904, 1974.
Kojima, Y., et al., J Biosci BioEng. 96(3):242-249, 2003.
Kokryakov, et al., FEBS Lett. 327; 231-36, 1993.
Kolakowski, J. E. et al., Biocatal. Biotransform. 15:297-312, 1997.
Kollar, R., et al., E. J. Biol. Chem., 270, 1170-1178, 1995.
Komives, C. et al., Biotechnol. Prog. 10:340-343, 1994.
Kontkanen, H. et al., Biotechnol BioEng. 94(3):407-415, 2006.
Koo, et al., Biochim. Biophys. Acta 1382; 80-90, 1998.
Korn, E. D. and Quigley., J. Biol. Chem. 226: 833-839, 1957.
Kraemer, F. B. et al., J Lipid Res. 34(4):663-671, 1993.
Kumar A., Gross R., Journal of American Chemical Society 122:11767-11770, 2000.
Kuo, J. M. and Raushel, F. M., Biochemistry 33:4265-4272, 1994.
Kurt Faber, “Biotransformations in Organic Chemistry, a Textbook, Third Edition.” Springer-verlag Berlin Heidelberg, pp. 114-115, 1997.
Kyte, et al., J. Mol. Biol., 157:105-32, 1982.
Kyte, J. and Doolittle, R. F. J. Mol. Biol., 157:105-132, 1982.
Laane, C., Boeren, S., Vos, K., Veeger, C., Biotechnology and Bioengineering 30:81-87, 1987.
Lai, K. et al., Arch. Biochem. Biophys. 318:59-64, 1995.
Lai, K. et al., J. Biol. Chem. 269:16579-16584, 1994.
Lalonde, J. J. et al., J. Am. Chem Soc 117:6845-6852, 1995.
Lamberty, et al., Biochemistry 40; 11995, 2001.
Lamberty, et al., J. Biol. Chem. 376; 4085-92, 2001.
Lambourne, et al., Eds., Paint and Surface Coatings, Theory and Practice, Second Edition, pp. 193-94, 371-82, and 543-47, 1999.
Landis, W. G. et al., J. Appl. Toxicol. 7:35-41, 1987.
Landrock, A. H., Adhesives Technology Handbook, Noyes Publications, New Jersey, 1985.
Langston, T. B. et al., Lipids. 40(1):31-38, 2005.
Leduc, M. et al., J. Bacteriol. 161:627-635, 1985.
Lee, C. Y. et al., Biochemistry. 46(51):14969-14978, 2007.
Lee, et al., Biol. Pharm. Bull. 18; 1049-52, 1995.
Lee, et al., Protein Pept Lett 9(5); 395-402, 2002.
Lee, J. Y., et al., Biotech. & Bioeng. 43:1146-1152 (1994).
Lee, L. C. et al., J Agric Food Chem.55(13):5103-5108, 2007.
Lee, S. W. et al., Appl Microbiol Biotechnol. 65(6):720-726, 2004.
Lehman, N. et al., FASEB J. 21(4):1075-1087, 2007.
Lei, C. et al., J Am Chem Soc 124:11242-11243, 2002.
LeJeune, K. E. and Russell, A. J. Biotech. and Bioeng. 51(4):450-457, 1996.
LeJeune, K. E. and Russell, A. J., Biotech and Bioeng 62(6):559-665, 1999.
LeJeune, K. E. et al., Ann. NY Acad. Sci. 864:153-170, 1998a.
LeJeune, K. E. et al., Biotechnology and Bioengineering 54(2):105-114, 1997.
LeJeune, K. E. et al., Biotechnology and Bioengineering 64(2):250-254, 1999.
LeJeune, K. E., Wild, J. R., Russell, A. J. “Nerve agents degraded by enzymatic foams” 395(6697):27-28, 1998b.
Lenz, D. E. et al., Biochim Biophys. Acta, 321:189-196, 1973.
Leow, T. C. et al., Biosci Biotechnol Biochem. 68(1):96-103, 2004.
Leung, A. K. et al., Biochemisty 40:5665-5673, 2001.
Levisson, M. et al., FEBS J. 274(11):2832-2842, 2007.
Lewis, V. E. et al., Biochemistry 27:1591-1597, 1988.
Li, H., and Zhang X. Protein Expr Purif. 42(1):153-159, 2005.
Li, H., Zhang X. et al., Protein Expr Purif. 42(1):153-159, 2005.
Li, S. et al. J Biochem (Tokyo) 124:332-339, 1998.
Li, S. L. et al. J. Bacteriol. 172:6506-6511, 1990.
Li, W.-S. et al., Bioorganic & Medicinal Chemistry, 9:2083-2091, 2001.
Liepinsh, et al., Nat Struct Biol. 4; 793, 1997.
Lineweaver, H. and Burke, D. “J. Am. Chem. Soc. 56:658-666, 1934.
Linke, T. et al., J Biol. Chem. 280(24):23287-23294, 2005.
Little, J. S. et al., Biochem Pharmacol 38(1):23-29, 1989.
Lo, M. et al., Plant Physiol. 135(2):947-958, 2004.
Lockridge, O. et al., Biochemistry 36:786-795, 1997.
Loessner, M. J. et al., Appl Environ Microbiol 62:3057-3060, 1996.
Lopez, M. et al., Blood 92(12):4602-4611, 1998.
Lopez, R. et al., Res Microbiol 151:437-443, 2000.
Luo, C. et al., Biochemistry 38:9937-9947, 1999.
Luo, Y. et al., Appl Microbiol Biotechnol. 73(2):349-355, 2006.
Lynn, W. S, and Perryman, N.C. J. Biol. Chem. 235:1912-1916, 1960.
Ma, C. et al., Parasitol Res. 101(2):419-425, 2007.
Ma, J. et al., Protein Expr Purif. 45(1):22-29, 2006.
Mackness, M. I. et al., Biochem. J. 245:293-296, 1987.
Mahapatro, A., Kalra, B., Kumar, A., Gross, R. A., Biomacromolecules 4:544-551, 2003.
Mahapatro, A., Kumar, A., Kalra, B., Gross, R. A., Macromolecules 37:35-40, 2004.
Main, A. R., Biochem J. 74:10-20, 1960.
Mainwaring, D. O. et al. Biotechnol 75:1-10, 1999.
Mak, et al., Infect. Immun. 64; 4444-49, 1997.
Makrides, S. C. Microbiol. Rev. 60:512-538, 1996.
Manco, G. et al., Arch Biochem Biophys. 373(1):182-192, 2000.
Manco, G. et al., Biochem J. 332 (Pt 1):203-212, 1998.
Mandard, et al., Eur J. Biochem. 256; 404, 1998.
Mandard, et al., Eur. J. Biochem. 269; 1190, 2002.
Mandard, et al., J. Biomol. Struct. Dyn. 17; 367, 1999.
Mandrich, L. et al., Archaea. 2(2):109-115, 2007.
Mansfeld, J. et al., Biochemistry. 45(18):5687-5694, 2006.
Martinek, K. et al., Biochim. Biophys. Acta. 485:1-12, 1977.
Martinez, C. et al., Biochemistry, 33:83-89, 1994.
Martinez, M. B. et al., Biochem 35(4):1179-1186, 1996.
Martinez, M. B. et al., Biochem 40(40):11965-11974, 2001.
Martins, et al., J. Mol. Biol. 258; 322, 1996.
Masaki, T. et al., Biochim. Biophys. Acta 660:51-55, 1981.
Masaki, T. et al., Biochim. Biophys. Acta. 660:44-50, 1981.
Masayama, A. et al., J. Bacteriol. 189(6):2369-2375, 2007.
Masschalck, B. and Michiels, C. W. Crit. Rev Microbiol. 29:191-214, 2003.
Masson, P. et al., J. Physiology (Paris), 92:357-362, 1999.
Matos, A. R. et al., Biochem Soc Trans. 28(6):779-781, 2000.
Matsui, K. et al., FEBS Lett. 569(1-3):195-200, 2004.
McCabe, R., Taylor, A., Tetrahedron 60:765-770, 2004.
McClellan, J. S. et al., Eur. J. Biochem. 258:419-429, 1998.
McDaniel, S, and Wild, J. Arch. Env. Contam. Toxic. 17:189-194, 1988.
McDaniel, S. et al., J. Bact. 170:2306-2311, 1988a.
McDaniel, S., Ph.D. Dissertation, Texas A&M University, 1985.
McGuinn, W. D. et al., Fundamental and Applied Toxicology 21:38-43, 1993.
McTiernan, C. et al., Proc. Natl. Acad. Sci. 84:6682-6686, 1987.
Mehrotra, K. N., and Phokela, A., Indian J. Entomol. 34:355-358, 1974.
Melo, E. P. et al., Biochemistry, 34(5):1615-1621, 1995.
Mentlein, R. et al., Arch. Biochem. Biophys. 200:547-559, 1980.
Michalowski, et al., submitted to EMBL GenBank DDBJ databases, 1998.
Michel, G. et al. Acta Crystallogr. D Biol. Clystallogr. 55:918-920, 1999.
Michel, G. et al. J. Biol. Chem. 276:40202-40209, 2001.
Michel, G. et al. J. Mol. Biol. 334:421-433, 2003.
Michel, G., et al., Structure 9:513-525, 2001.
Millard, C. B. et al., Biochemistry 37(1):237-247, 1998.
Millard, C. B. et al., Biochemistry, 38:7032-7039, 1999.
Millard, C. B. et al., Biochemistry. 34(49):15925-15933, 1995.
Millard, C. B. et al., Biochemistry. 34(49):15925-15933., 1995.
Miller, C. E. Ph.D. dissertation, Texas A&M University, 1992.
Mizuguchi, S. et al., J. Biochem. 126(4):731-737, 1999.
Mogelson, S, and Lange, L. G. Biochemistry 23:4075-4081, 1984.
Moore, et al., J. Biol. Chem. 266; 19851-57, 1991.
Moore, G. J., Trends Pharmacol. Sci., 15:124-129, 1994.
Mooreman, et al., Eur. J. Biochem. 269; 4799-810, 2002.
Mor, et al., Eur J Biochem 219(1-2); 145-54, 1994.
Mor, et al., Proc. Natl. Acad. Sci. USA 91; 10295-99, 1994.
Moraleda-Muñoz, A. and Shimkets, L. J. J. Bacteriol. 189(8):3072-3080, 2007.
Morana, A. et al., Gene. 283(1-2):107-115, 2002.
Moreau, R. A. and Huang, A. H. C. Methods Enzymol. 71:804-813, 1981.
Mori, T. et al., Enzymologia, 43:213-226, 1972.
Morrison, M. et al., J. Biol. Chem. 228:767-776, 1957.
Mosbah, H. et al., Protein Expr Purif. 47(2):516-523, 2006.
Mosbah, H. et al., Protein Expr Purif. 55(1):31-39, 2007.
Mulbry, W. and Karns, J., J. Bacteriol. 171:6740-6746, 1989.
Mulbry, W. et al., Appl. Env. Micro. 51:926-930, 1986.
Mulchandani, A. et al., Anal Chem 70:4140-4145, 1998b.
Mulchandani, A. et al., Anal Chem 70:5042-5046, 1998c.
Mulchandani, A. et al., Biosensors & Bioelectronics 16:225-230, 2001.
Mulchandani, A. et al., Biotechnol. Progr 5:130-134, 1999a.
Mulchandani, A. et al., Biotechnology and Bioengineering 63(2):216-223, 1999b.
Mulchandani, A. et al., Electroanalysis 10:733-737, 1998a.
Mulchandani, P. et al., Biosensors & Bioelectronics 14:77-85, 1999.
Mulchandani, P. et al., Biosensors & Bioelectronics 16:433-437, 2001b.
Mulchandani, P. et al., Environ Sci Technol 35:2562-2565, 2001a.
Munford, R. S, and Hunter, J. P. J. Biol. Chem. 267:10116-10121, 1992.
Munnecke, D. M., Biotechnol. Bioeng. 21:2247-2261, 1979.
Munnecke, D. M., Process Biochemistry 13:14-16, 31, 1978.
Murasugi, A. et al., Protein Expr Purif. 23(2):282-288, 2001.
Murphy, John “Additives for Plastics Handbook 2^{nd} Edition,” Elsevier Science Ltd. Kidlington, Oxford OX5 1 GB, UK, 2001.
Myers, F. L. and Northcote, D. H., Biochem. J. 71:749-756, 1959
Nagaoka, et al., DNA Seq. 4; 123-28, 1993.
Nagaoka, et al., FEBS Lett. 280; 287-91, 1991.
Nakahigashi, K. and Inokuchi, H. Nucleic Acids Res. 18(21):6439, 1990.
Narita, J. et al., Appl Microbiol Biotechnol. 70(5):564-572, 2006. Nato Army Armaments Group Project Group 31 on Non-Corrosive, Biotechnology-Based Decontaminants for CBW Agents, Decision Sheet AC/225(PG/31)DS(2002)2, 26 Sep. 2002.
Nauze, M. et al., “J Biol Chem. 277(46):44093-44099, 2002.
Nedkov, P., et al. Biol. Chem. Hoppe-Seyler 366:421-430, 1985.
Neugnot, V. et al., Eur J. Biochem. 269(6):1734-1745, 2002.
Newcomb, R. D. et al., Proc. Natl. Acad. Sci. USA 94:7464-7468, 1997.
Nicaud, J. M. et al., FEMS Yeast Res. 2(3):371-379, 2002.
Nicolas, A. et al., Biochemistry, 35:398-410, 1996.
Nieuwenhuizen, W. F. et al., Protein Expr Purif. 30(1):94-104, 2003.
Nijs, M. et al., Appl Biochem Biotechnol 49:75-91, 1994.
Nikoleit, K. et al., Eur J. Biochem. 228(3):732-738, 1995.
Nishiwaki, H. et al., Eur J Biochem. 271(3):601-606, 2004.
Niwa, t., et al., J. Microbiol. Methods, 61, 251-260, 2005.
Nthangeni, M. B. et al., Enzyme Microb Technol. 28(7-8):705-712, 2001.
O'Flaherty, S. et al., J. Bacteriol 187:7161-7164, 2005.
Ogino, C. et al., Appl Microbiol Biotechnol. 64(6):823-828, 2004.
Ogino, H. et al., Extremophiles. 11(6):809-817, 2007.
Ogino, H. et al., J Mol Microbiol Biotechnol. 7(4):212-223, 2004.
Oh, I. S. et al., Plant Cell. 17(10):2832-2847, 2005.
Ohara, T. et al. J. Biol. Chem. 264:20625-2063, 1989.
Ohbuchi, K. et al., J. Biosci.Bioeng.91:487, 2001.
Ohta, Y. and Hatada, Y. J. Biochem. (Tokyo) 140:475-481, 2006.
Ohta, Y. et al. Biosci. Biotechnol. Biochem. 68:1073-1081, 2004a.
Ohta, Y. et al. Microbulbifer. Appl. Microbiol. Biotechnol. 64:505-514, 2004b.
Ohta, Y., et al. Curr. Microbiol. 50:212-216, 2005.
Ohto, T. et al., J Biol Chem. 280(26):24576-24583, 2005.
Okawa, Y. and Yamaguchi, T. J. Biochem. (Tokyo) 81:1209-1215, 1977.
Okazaki, H. et al., J Biol Chem. 277(35):31893-31899, 2002.
Okino, N. et al., J Biol Chem. 274(51):36616-36622, 1999.
Ollis, D. L. et al., Protein Engineering 5:197-211, 1992.
Olson, et al., Biochem. Biophys. Res. Commun. 288; 1001-05, 2001.
Omburo, G. A. et al., Biochemistry 32:9148-9155, 1993.
Omburo, G. A. et al., J. Biol. Chem. 267:13278-13283, 1992.
Oppenheim, et al., J. Biol. Chem. 263; 7472-77, 1988.
Orivel, et al., J. Biol. Chem. 276; 17823-29, 2001.
P. W. Atkins, The Elements of Physical Chemistry, 3rd edition, Oxford University Press, p. 114, 1993.
Palomo, J. M. et al., Biotechnol Prog. 20(2):630-635, 2004.
Pariyarath, R. et al., FEBS Lett. 397(1):79-82, 1996.
Park, et al., Biochem. Biophys. Res. Commun. 205; 948-54, 1994.
Park, et al., Biochem. Biophys. Res. Commun. 218; 408-13, 1996.
Park, et al., FEBS Lett 507(1); 95-100, 2001.
Park, et al., FEBS Lett. 411; 173-78, 1997.
Park, Y. J. et al., Biochim Biophys Acta. 2006 1760(5):820-828, 2006.
Passolunghi, S. et al., Biotechnol Lett. 25(22):1945-1948, 2003.
Paul, K. G. Peroxidases. In: Boyer, P. D., Lardy, H. and Myrbäck, K. (Eds.), The Enzymes, 2nd ed., vol. 8, Academic Press, New York, p. 227-274, 1963.
Peanasky, R. J. et al., Biochim. Biophys. Acta 181:82-92, 1969.
Pei, L. et al., Toxicology and Applied Pharmacology 124:296-301, 1994.
Persichetti, R. A. et al., Tetrahedron Lett, 37:6507-6510, 1996.
PesaResi, A. et al., Curr Microbiol. 50(2):102-109, 2005.
Petersen, E. I. et al., J Biotechnol. 89(1):11-25, 2001.
Petit, J., et al., Trends Genet., 10:4-5, 1994.
Petrie, E. M., Handbook of Adhesives and Sealants, McGraw-Hill, New York, 2000.
Petrie, E. M., Handbook of Adhesives and Sealants, McGraw-Hill, New York, 2000.
Petrikovics, I. et al., Toxicology and Applied Pharmacology 156:56-63, 1999.
Petrikovics, I. et al., Drug Delivery 7:83-89, 2000b.
Petrikovics, I. et al., Toxicological Sciences 57:16-21, 2000a.
Phillips, J. P. et al., Proc. Natl. Acad. Sci. U.S.A. 87:1-5, 1990.
Pierce, R. J. et al. Biochem. J. 180:673, 1979.
Pierce, R. J. et al. Biochem. J. 185:261-264, 1980.
Plueddemann, Edwin, P. “Silane Coupling Agents,” Plenum Press, New York, 1982.
Plummer, T. H., Jr. and Tarentino, A. L. J. Biol. Chem. 256:10243-10246, 1981.
Polgár, L. Structure and function of serine proteases. In New Comprehensive Biochemistry Vol. 16, Hydrolytic Enzymes (Neuberger, A. and Brocklehurst, K. eds), pp. 159-200, Elsevier, Amsterdam (1987).
Polgár, L. Structure and function of serine proteases. In New Comprehensive Biochemistry Vol. 16, Hydrolytic Enzymes (Neuberger, A. and Brocklehurst, K. eds), pp. 159-200 Elsevier, Amsterdam, 1987
Pope, J. L. et al., J. Biol. Chem. 241:2306-2310, 1966.
Potin, P. et al. Eur. J. Biochem. 201; 241-247, 1991.
Potin, P. et al. Eur. J. Biochem. 228:971-975, 1995.
Potin, P., et al. Eur. J. Biochem. 214:599-607, 1993.
Powell, A. J. et al., J. Mol. Biol. 359:122-136, 2006.
Powell, M. F. et al., Pharma. Res., 10:1268-1273, 1993.
Prathumpai, W. et al., Appl Microbiol Biotechnol. 65(6):714-719, 2004.
Primrose, S. et al., “Principles of Gene Manipulation” pp. 301-303, 2001.
Purdy, R. E. and Kolattukudy, P. E. Biochemistry 14:2824-2831, 1975.
Qin, C. et al., Biochim Biophys Acta. 1761(12):1450-1458, 2006.
Qu, et al., Eur. J. Biochem. 127; 219-24, 1982.
Quintero, C. et al., Progress in Organic Coatings, 57:195-201, 2006.
Quintero, C., et al., Progress in Organic Coatings, 57:202-209, 2006.
Qureshi, N. et al., Appl. Microbiol. Biotechnol. 21:280-281, 1985.
Quyen, D. T. et al., Protein Expr Purif. 28(1):102-110, 2003.
Rahman, R. N. et al., Protein Expr Purif. 40(2):411-416, 2005.
Rainina, E. I. et al., Biosensors Bioelectronics 11:991-1000, 1996.
Raj, et al., Biopolymers 45(1); 51-67; 1998.
Rashid, N. et al., Appl Environ Microbiol 0.67(9):4064-4069, 2001.
Rashid, S. et al., Circulation. 107(24):3066-3072, 2003.
Rastogi, V. et al., Biochem. and Biophys. Research Comm. 241:294-296, 1997.
Raushel, Frank M., Microbiology, 5:288-295, 2002.
Raveh, L. et al., Biochemical Pharmacology 44(2):397-400, 1992.
Ravi, et al., J. Biol. Chem. 272; 24480-87, 1997.
Read, R. J. et al., Biochemistry 23:6570-6575, 1984.
Rebuffat, et al., Eur. J. Biochem. 201; 661-74, 1991.
Recsei, P. A., et al. Proc. Natl. Acad. Sci. U.S.A. 84:1127-1131, 1987
Reese, E. T. and Mandels, M. Can. J. Microbiol. 5:173-185, 1959.
Reese, E. T. and Mandels, M. Can. J. Microbiol. 5:173-185, 1959.
Reetz, M. et al., Aggew Chen Int Ed Engl 34:301-303, 1995.
Reid, C. W. et al., FEBS Lett 574:73-79, 2004.
Resina, D. et al., Biotechnol BioEng. 91(6):760-767, 2005.
Resina, D. et al., J Biotechnol. 109(1-2):103-113, 2004.
Resina, D. et al., Microb Cell Fact. 6:21, 2007.
Rhee, J. K. et al., Appl Environ Microbiol. 71(2):817-825, 2005.
Rhee, S. G. and Bae, Y. S. J. Biol. Chem. 272:15045-15048, 1997.
Richins, R. D. et al., Biotechnology and Bioengineering 69(6):592-596, 2000.
Richins, R. D. et al., Nature Biotechnology 15:984-987, 1997.
Rizzarelli, P., Impallomeni, G., Montaudo, G., Biomacromolecules 5:433-444, 2004.
Rizzo, M. et al., Arterioscler Thromb Vasc Biol. 24(1):141-146, 2004.
Robinette, et al., Cell. Mol. Life. Sci. 54; 467-75, 1998.
Rodrigo, L. et al., Biochem J. 321:595-601, 1997.
Rogers, K. R. et al., Biotech Prog 15:517-521, 1999.
Rogers, S. G. et al., Enzymology 153: 253-292, 1987.
Roon, R. J. and Levenberg, B. Methods Enzymol. 17A:317-324, 1970.
Rouette, H. K., Encyclopedia of Textile Finishing, Springer-Verlag, Berlin Heidelberg, 2001.
Rouette, H. K., Encyclopedia of Textile Finishing, Springer-Verlag, Berlin Heidelberg, 2001.
Roustan, J. L. et al., Appl Microbiol Biotechnol. 68(2):203-212, 2005.
Rowland, S. S. et al., Appl Environ Microbiol 57(2):440-444, 1991.
Rowland, S. S. et al., Appl. Microbiol. Biotechnol. 38:94-100, 1992.
Roy, A. B. Adv. Enzymol. Relat. Subj. Biochem. 22:205-235, 1960.
Roy, A. B. Aust. J. Exp. Biol. Med. Sci. 54:111-135, 1976.
Rozek, et al., Biochemistry 39; 15765, 2000.
Rozek, et al., Eur. J. Biochem. 267; 5330-41, 2000.
Rúa, M. L. et al., Appl Microbiol Biotechnol. 49(4):405-410, 1998.
Ruissen, et al., Peptides 2002 23(8); 1391-99, 2002.
Rupley, J. A., Biochim. Biophys. Acta, 83:245-255, 1964.
Rusnak, M. et al., Biotechnol Lett. 27(11):743-748, 2005.
Russell, R. J. et al., Analytical Chemistry 71:4909-4912, 1999.
Ryu, Y. et al., Biochim Biophys Acta. 1628(3):206-210, 2003.
Sahasrabudhe, A. V. et al., Protein Expr Purif. 14(3):425-433, 1998.
Sahoo, B., et al., Biomacromolecules 7:1042-1048, 2006.
Saito, K. and Hanahan, D. J. Biochemistry 1:521-532, 1962.
Sakuradani, E. et al., Eur. J. Biochem. 261:812-820, 1999.
Salazar, O. et al, Mol Biotechnol 33:211-220, 2006.
Sambasivam, M., Klein, A., Sperling, L. H., Journal of Applied Polymer Science 58 (2):357-366, 1995.
Sanchez, M. et al., Biotechnol BioEng. 78(3):339-345, 2002.
Satriana, M. J., Hot Melt Adhesives: Manufacture and Applications, Noyes Data Corporation, New Jersey, 1974.
Satriana, M. J., Hot Melt Adhesives: Manufacture and Applications, Noyes Data Corporation, New Jersey, 1974.
Sayari, A. et al., Mol. Biotechnol. 36(1):14-22, 2007.
Scharff, E. I. et al., Acta Cryst. D57:148-149, 2001.
Scherrer, R., Trends Biochem. Sci. 9: 242-245, 1984.
Scheurwater, E. et al. Int. J. Biochem. Cell Biol, 2007.
Schibli, et al., Biochemistry 38; 16749, 1999.
Schlieben, N. H. et al., Protein Expr Purif. 34(1):103-110, 2004.
Schmidt, J. A. et al., J. Bacteriol. 186(17):5790-5798, 2004.
Schmidt-Dannert, C. et al., Biochim Biophys Acta. 1301(1-2):105-114, 1996.
Schonwetter, et al., Science 267; 1645-48, 1995.
Scoochi, et al., FEBS Lett. 417; 311-15, 1997.
Scott, J. H. and Schekman, R. et al., J. Bacteriol 142:414-423, 1980.
Sebastian, J., and Kolattukudy, P. E. Arch Biochem Biophys. 263(1):77-85, 1988.
Sebban-Kreuzer, C. et al., Protein Expr Purif. 49(2):284-291, 2006.
Segel, I. H. Biochemical Calculations: How to Solve Mathmatical Problems in General Biochemistry 2^{nd} Edition, John Wiley & Sons, Inc., New York, 1976.
Selsted, et al., J. Biol Chem. 264; 4003-07, 1989.
Selsted, et al., Proc. Natl. Acad. Sci. USA 85; 592-96, 1988.
Sendak, R. A., and Bensadoun A. J Lipid Res. 39(6):1310-1315, 1998.
Seo, K. H., Rhee J I. Biotechnol Lett. 26(19):1475-1479, 2004.
Serdar, C. M. and Gibson, D. BiolTechnology 3:567-571, 1985.
Serdar, C. M. et al., Appl Environ Microbiol 44:246-249, 1982.
Serdar, C. M. et al., BiolTechnology 7:1151-1155, 1989.
Shafferman, A. et al., Biochem. J. 318:833-840, 1996.
Sheiknejad, R. G. and Srivastava, P. N. J. Biol. Chem. 261:7544-7549, 1986.
Shen, S. H. et al., J Biol Chem 266:1058-1063, 1991.
Shiba, Y. et al., Biosci Biotechnol Biochem. 65(1):94-101, 2001.
Shim, H. et al., J. Biol. Chem. 273(28):17445-17450, 1998.
Shimazu, M. et al., Biotech and Bioeng 76(4):318-324, 2001b.
Shimazu, M. et al., Biotechnol. Prog. 17:76-80, 2001a.
Shimazu, M. et al., Biotechnology and Bioengineering 81(1):74-79, 2002.
Shimoi, H. et al, J. Biol. Chem. 267:25189-25195, 1992.
Shu, Z. Y. et al., Biotechnol Lett. 29(12):1875-1879, 2007.
Sias, B. et al., Biochemistry. 43(31):10138-10148, 2004.
Silver, F. et al., J. Long-Term Effects Med. Implants 1:329, 1992.
Sinchaikul, S. et al., Acta Crystallogr D Biol Crystallogr. 58(Pt 1):182-185, 2002.
Sinchaikul, S. et al., Biochem Biophys Res Commun. 283(4):868-875, 2001.
Singh, A. K. et al., Biosensors & Bioelectronics 14:703-713, 1999.
Skeist, I., ed., Handbook of Adhesives, 3^{rd} Ed., Van Nostrand Reinhold, New York, 1990.
Skeist, I., Ed., Handbook of Adhesives, 3^{rd} Ed., Van Nostrand Reinhold, New York, 1990.
Skerlavaj, et al., J. Biol Chem. 271; 28375-81, 1996.
Slade, P. E., et al., “Handbook of Fiber Finish Technology,” Marcel Dekker (1998).
Slade, P. E., et al., Handbook of Fiber Finish Technology, Marcel Dekker, 1998.
Slusarski, L., ed., Fillers for the New Millenium-Macromolecular Symposia 194, Wiley-VCH, Verlag, 2003.
Smith, S. W., et al. J. Biol. Chem. 228:915-922, 1957.
Smith, T. J. et al., Microbiology 146:249-262, 2000.
Soedjanaatmadja, et al., Biochim. Biophys. Acta 1209; 144-48, 1994.
Somara, S. et al., Indian J Exp Biol 40(7):774-779, 2002.
Sonesson, A. W., Callisen, T. H., Brismar, H., Elofsson, U. M., Langmuir 21:11949-11956, 2005.
Sonesson, A. W., Elofsson, U. M., Brismar, H., Callisen, T. H., Langmuir 22 (13), 5810-5817, 2006.
Song, H. T. et al., Protein Expr Purif. 47(2):393-397, 2006.
Song, J. K et al., J Biotechnol. 72(1-2):103-114, 1999.
Song, J. K. et al., J Biotechnol. 130(3):311-315, 2007.
Soreq, H. et al., Proc. Natl. Acad. Sci. 87:9688-9692, 1990.
Soucek, A., et al., Biochim. Biophys. Acta 227:116-128, 1971.
Soya, V. V., Elyakova, L. A. and Vaskovsky, V. E. Biochim. Biophys. Acta 212:111-115, 1970.
Srivastava, R. et al., Applied Environ Microbio 66(10):4366-5371, 2000.
Standard Practice for Cyclic Salt Fog/UV Exposure of Painted Metal, (Alternation Exposures in a Fog/Dry Cabinet and a UV/Condensation Cabinet) (ASTM D5894-96).
Standard Practice for Modifies Salt Spray (Fog) Testing. Appendix A5: Dilute Electrolyte Cyclic Fog/Dry Test (ASTM G85-94).
Standard Practice for Operating Light and Water-Exposure Apparatus (Fluorescent UV-Condensation Type) for Exposure of Nonmetallic Materials. (ASTM G53-88).
Standard Test Method for Evaluation of Painted or Coated Specimens Subjected to Corrosive Environments (ASTM D1654-92).
Steiert, J. G. et al., BiolTechnology 7:65-68, 1989.
Steurbaut, W., DeKimpe, N., Schreyen, L., Dejonckheere, W. Bull Soc. Chim Belg. 84:791, 1975.
Stevens, R. C. Structure Fold Des. 8(9):R177_R185, 2000.
Storkebaum, W. and Witzel, H., Forschungsber. Landes Nordrhein-Westfalen 2523:1-23, 1975.
Stoye, et al., Eds., Paints, Coatings and Solvents, Second, Completely Revised Edition, pp. 6, 127, and 165, 1988.
Suen, W. C. et al., Protein Eng Des Sel. 17(2):133-140, 2004.
Sueyoshi, N. et al., J. Bacteriol. 184(2):540-546, 2002.
Sugano, Y. et al. Appl. Environ. Microbiol. 59:1549-1554, 1993.
Sugano, Y., et al. J. Bacteriol. 176:6812-6818, 1994.
Sugihara, A. et al., J Biochem, 112(5):598-603, 1992.
Sulong, M. R. et al., Protein Expr Purif. 49(2):190-195, 2006.
Sussman, J. S. et al., Science 253:872-879, 1991.
Tadmor, Z. and Costas, G. G. “Principles of Polymer Processing Second Edition,” John Wiley & Sons, Inc.
Hoboken, N. J., 2006.
Tagawa, K. et al., Nature (Lond.) 183:111, 1959.
Tai, T. et al. J. Biol. Chem. 250:8569-8575, 1975.
Takahashi, N. and Nishibe, H. J. Biochem. (Tokyo) 84:1467-1473, 1978.
Takahashi, N. Biochem. Biophys. Res. Commun. 76:1194-1201, 1977.
Takahashi, T., et al., Biochim. Biophys. Acta 351:155-171, 1974.
Tamalampudi, S. et al., Appl Microbiol Biotechnol. 75(2):387-395, 2007.
Tamura, H. et al., J. Biochem. 112(4):488-491, 1992.
Tan, C. A. et al., Protein Expr Purif. 10(3):365-372, 1997.
Tang, et al., Infect. Immun. 67; 6139-44, 1999.
Tang, S. J. et al., Arch Biochem Biophys. 387(1):93-98, 2001.
Tani, T. and Tominaga, Y. J. Biochem., 109(2):211-216, 1991.
Tani, T., et al. Nucleic Acids Res. 18:1631, 1990.
Tarentino, A. L. et al. Biochemistry 24:4665-4671, 1985.
Tarentino, A. L., et al. J. Biol. Chem. 249:818-824, 1974.
Tchelet, R. et al., Soil. Biol. Biochem. 25:1665-1671, 1993.
Teo, J. W. et al., Gene. 312:181-188, 2003.
Terras, et al., FEBS Lett. 316; 233-40, 1993.
Theil, et al., EMBO J. 2; 1159-63, 1983.
Theorell, H. Ark. Kemi Mineral. Geol. 16A No. 2. 11 pp, 1943.
Thongekkaew, J., Boonchird C. FEMS Yeast Res. 7(2):232-243, 2007.
Thumm, G. and Götz, F. Mol. Microbiol. 23:1251-1265, 1997.
Thunnissen, A. M. et al. Nature 367:750-753, 1994.
Tinoco, et al., VOL. 277, No. 39; 36351-56, 2002.
Toke, D. A. et al., J Biol. Chem. 273(23):14331-14338, 1998.
Tomasek, P. H., et al., J. Bacteriology, 171(7):4038-4044 (1989).
Tomita, N. et al., Biochem. Biophys. Res. Commun. 158:569-575, 1989.
Touch, V. et al. J Agric Food Chem. 51:5154-5161, 2003.
Tracey, M. V. Biochem. J. 61:579-586, 1955.
Trayer, H. R., and Buckley, C. E., J. Biol. Chem., 245, 4842-4846, 1970.
Trimble, R. B. et al., Glycobiology. 14(3):265-274, 2004.
Tsuchiya, D., and Taga, M., Phytopathology, 91, 354-360, 2001.
Tsujita, T. et al., J. Lipid Res. 30:997-1004, 1989.
Tsunasawa, S. et al. J. Biol. Chem. 264:3832-3839, 1989.
Tuovinen, K. et al., Fundam Appl. Toxicol 23:578-584, 1994.
U.S. Pat. No. 4,244,693
U.S. Pat. No. 5,391,649
U.S. Pat. No. 5,602,097.
U.S. Pat. No. 5,882,731.
U.S. Pat. No. 5,885,782.
U.S. Pat. No. 5,919,689
U.S. Pat. No. 6,001,913
U.S. Pat. No. 6,020,312.
U.S. Pat. No. 6,203,720
U.S. Pat. No. 6,174,948
U.S. Pat. No. 6,235,916
U.S. Pat. No. 6,599,972
U.S. Pat. No. 6,624,223
U.S. Pat. No. 6,653,381
U.S. Pat. No. 6,700,006
U.S. Pat. No. 6,897,255
U.S. Pat. No. 6,897,257
U.S. Pat. No. 6,913,628
U.S. Patent Publication no. 20050203246 A1
U.S. Patent Publication no. 20060211795 A1
U.S. Patent Publication no. 20060236467 A1
U.S. Patent Publication no. 20080183000 A1
Ueta, et al., J Pept Res 2001 57(3); 240-49, 2001.
Ugaki, M. et al., Nucl. Acid Res., 19:371-377, 1991.
Umemura, I. et al., Appl. Microbiol. Biotechnol. 20:291-295, 1984.
Unger, T. F. The Scientist, 11(17):20, 1997.
Urry, D. W., Prog Biophys Mol Bio157:23-57, 1992.
U.S. patent application Ser. No. 10/601,207
US. Patent Publication no. US 2002/0106361 A1
van Asselt, E. J. et al., J Mol. Biol. 291:877-898, 1999a.
van Asselt, E. J. et al., Structure Fold Des 7:1167-1180, 1999b.
van den Bosch, H., et al., Biochim. Biophys. Acta 296:94-104, 1973.
van den Bosch, H., et al., Methods Enzymol. 71:513-521, 1981.
Van der Mee, L., Helmich, F., Bruijn, R., Vekemans, J., Palmans, A., Meijer, E. W., Macromolecules 39:5021-5027, 2006.
van der Wal, F. J. et al., Appl. Environ. Microbiol., 64(2):392-398, 1998.
Van Hamme, J. D. Microbiology and molecular biology reviews, 67(4):503-549, 2003.
van Straaten, K. E. et al., J. Biol. Chem. 282:21197-21205, 2007.
van Straaten, K. E. et al., J. Mol. Bio1352:1068-1080, 2005.
Vanhooke, J. L. et al., Biochemistry 35:6020-6025, 1996.
Varma, I. K., Albertsson, A., Rajkhowa, R., Srivastava, R. K., Progress in Polymer Science 30:949-981, 2005.
Venezuela patent application USM:042VE
Venkataraman, G. et al., Mol Genet Genomics. 270(5):378-386, 2003.
Ventom, A. M. and Asenjo, J. A. J. Biotechnol Tech 4:171-176, 1990.
Vertommen, M. A. M. E., Nierstrasz, V. A., Van der Veer, M., Warmoeskerken, M. M. C. G., Journal of Biotechnology 120:376-386, 2005.
Vitarius, J. A. and Sultatos, L. G. Life Sciences, 56(2):124-134, 1995.
Vogle, et al., Biochem. Cell Biol. 80; 49-63, 2002.
Vontas, J. G., et al., Insect Molec. Bio., 11(4):329-336 (2002).
Walker, A. W. and Keasling, J. D. Biotech. and Bioeng. 78(7):715-721, 2002.
Walker, B. M., ed., Handbook of Thermoplastic Elastomers, Van Nostrand Reinhold Co., New York, 1979.
Walker, B. M., Ed., Handbook of Thermoplastic Elastomers, Van Nostrand Reinhold Co., New York, 1979.
Wall, G. Sigma-Aldrich Email communication 2006.
Wallace, T. J. et al., J Biol Chem. 276(35):33165-33174, 2001.
Walsh, K. A. Methods Enzymol. 19:41-63, 1970.
Walsh, S. B., et al., Biochem. J., 359:175-181 (2001).
Wan, E. W. and Baneyx, F. Protein Expr. Purif. 14(1):13-22, 1998.
Wang, A. A. et al., Applied and Environ. Microbio. 68(4):1684-1689, 2002.
Wang, B. et al., Protein Expr Purif. 35(2):199-205, 2004.
Wang, et al., Biochem. Biophys. Res. Commun. 279; 407-11, 2000.
Wang, et al., Biochem. Biophys. Res. Commun. 288; 765-70, 2001.
Wang, et al., J. of Controlled Release 17:23-25, 1991.
Wang, G. Y. et al., Fungal Genet Biol. 35(3):261-276, 2002.
Wang, H. T. et al., J. of Controlled Release 17:23-25, 1991.
Wang, I. N. et al., Annu Rev Microbiol 54:799-825, 2000.
Wang, J. et al., Biomacromolecules 2:700-705, 2001.
Ward, J. B. et al., J. Gen. Microbiol. 128:1171-1178, 1982.
Warth, A. H., The Chemistry and Technology of Waxes, Reinhold Publishing Corporation, New York, 1956.
Washida, M. et al., Appl Microbiol Biotechnol. 56(5-6):681-686, 2001.
Watkins, L. M. et al., J. Biol. Chem. 272(41):25596-25601, 1997a.
Watkins, L. M. et al., Proteins: Struct., Funct., and Gen. 29:553-561, 1997b.
Webb, E. C. and Morrow, P. F. W. Biochem. J. 73:7-15, 1959.
Weigl, J. and Yashe, W. Can. J. Microbiol. 12:939-947, 1966.
Whitaker, D. R. and Roy, C. Can. J. Biochem. 45:911, 1967.
Whitaker, D. R. et al. Can. J. Biochem. 43:1961-1970, 1965.
Whitaker, D. R. et al. Can. J. Biochem. Physiol. 41:671-696, 1963
White, S. R., Sottos, N. R., Geubelle, P. H., Moore, J. S., Kessler, M. R., Sriram, S. R., Brown, E. N., Viswanathan, S., Nature 409:794, 2001.
Whitehouse, L. W., and Ecobichon, D. J., Pestic. Biochem. Phys. 5:314-322, 1975.
Wicks, et al., Organic Coatings, Science and Technology, Volume 1: Film Formation, Components, and Appearance, pp. 318-20, 1992.
Wicks, et al., Organic Coatings, Science and Technology, Volume 2: Applications, Properties and Performance, pp. 145, 309, 319-23, and 340-41, 1992.
Wicks, Jr., Z. W., Jones, F. N., Pappas, S. P. “Organic Coatings, Science and Technology, Volume 1: Film Formation, Components, and Appearance” (1992) John Wiley & Sons, Inc., New York, U.S.A.
Wicks, Jr., Z. W., Jones, F. N., Pappas, S. P. “Organic Coatings, Science and Technology, Volume 2: Applications, Properties and Performance” (1992) John Wiley & Sons, Inc., New York, U.S.A.
Wierdl, M. et al., Biochem. Pharm. 59:773-781, 2000.
Wilcox, P. E. Methods Enzymol. 19:64-108, 1970.
Wild, J. R. et al., Proc. U.S. Army Chem. Res. Devel. Eng. Center Sci. Conf. Chem. Defense Res. 18-21
Nov., p. 629-634, 1986.
Wilde, et al., J. Biol. Chem. 264; 11200-03, 1989.
Wiley, R. A. and Rich, D. H. Med. Res. Rev., 13:327-384, 1993.
Wu, B. X. et al., J Lipid Res. 48(3):600-608, 2006.
Wu, C.-F. et al., Appl Microbiol Biotechnol 54:78-83, 2000b.
Wu, C.-F., Biotechnol. Prog. 17:606-611, 2001a.
Wu, C.-F., Biotechnology and Bioengineering 75(1):100-103, 2001 b.
Wu, C.-F., Biotechnology and Bioengineering 77(2):212-218, 2002.
Wu, F. et al., J. Am. Chem. Soc. 122:10206-10207, 2000a.
Wu, J. et al., Biochem J. 386(Pt 1):153-160, 2005.
Wu, M. et al., Lipids. 38(3):191-199, 2003.
Wypych, G. Handbook of Material Weathering, 2nd Ed. ChemTec Publishing. Canada 1995.
Xie, J., Hsieh, Y., Biocatalysis in Polymer Science ACS Symposium Series 840:217, 2002.
Xu, B. et al., J. Ferment. Bioeng. 81:473-481, 1996.
Xu, et al., J. Dent. Res 69; 1717-23, 1990.
Yamaguchi, S. et al., Biosci Biotechnol Biochem. 61(5):800-805, 1997.
Yang, F. et al., Biotechnol. Prog. 11:471-474, 1995.
Yang, J. et al., Protein Eng. 15(2):147-152, 2002.
Yang, Y.-C. et al., Chem. Rev. 92:1729-1743, 1992.
Yang, Y.-C. et al., J. Am. Chem. Soc. 112:6621-6627, 1990.
Yang, Y.-C. et al., J. Org. Chem. 61:8407-8413, 1996.
Yang, Z. et al., Biotechnol Bioeng 45:10-17, 1995.
Yang, Z. et al., Enzyme Microb Technol 18:82-89, 1996.
Yavin, E. and Gatt, S. Biochemistry 8:1692-1698, 1969.
Yi, et al., FEBS Lett. 398; 87-90, 1996
Yin, et al., Arch Oral Biol. 48(5); 361-68, 2003.
Yokogawa, K., et al., Agric. Biol. Chem., 39:1533-1545, 1975.
Yoshimoto, T. et al., J. Biochem. 105(3):412-416, 1989.
Yount, et al., J. Immunol. 155; 4476-84, 1995.
Yu, M et al., Protein Expr Purif. 53(2):255-263, 2007.
Zaks A., Klibanov A. M., Journal of Biological Chemistry 263:3194-3201, 1988.
Zaks A., Klibanov A. M., Proceedings to the National Academy of Science 82:3192-3196, 1985.
Zasloff, M. Proc. Natl. Acad. Sci. USA 84:5449-5453, 1987.
Zhai, S. et al., Biotechnol Lett. 27(11):799-804, 2005.
Zhang, et al., Biochemistry 31; 11348-56, 1992.
Zhang, F et al., Protein Expr Purif. 48(2):300-306, 2006.
Zhang, L. et al., J Biol Chem. 278(31):29344-29351, 2003.
Zhang, M. et al., Protein Expr Purif. 42(1):59-66, 2005.
Zhang, Y. et al., Biotech. Bioengineering 64:221-231, 1998.
Zhao, B. et al., Physiol Genomics. 23(3):304-310, 2005.
Zhao, et al., FEBS Lett. 346; 285-88, 1994.
Zhao, et al., FEBS Lett. 368; 197-202, 1995.
Zhong, Q. et al., J Agric Food Chem. 54(21):8086-8092, 2006.
Zhongli, C. et al., Applied and Environmental Microbiology 67(10):4922-4925, 2001.
Zhu, et al, Endocrinology 130; 1413-23, 1992.
Zhu, K. Y., et al., Insect Biochem. Mo/ec., 25(10):1129-1138 (1995).
Zimmermann, et al., Biochemistry 34; 13663, 1995.
Zosloff, Proc. Natl. Acad. Sci. USA, 84:5449-53, 1987.
Zschenker, O. et al., J. Biochem. 136(1):65-72, 2004.

What is claimed is:

1. A composition, comprising an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; wherein the composition comprises an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

2. The composition of claim 1, wherein the active enzyme comprises a plurality of active enzymes.

3. The composition of claim 1, wherein the enzyme comprises an esterase, a ceramidase, or a combination thereof, and wherein the esterase comprises a lipolytic enzyme, a phosphoric triester hydrolase, a sulfuric ester hydrolase, or a combination thereof.

4. The composition of claim 3, wherein the lipolytic enzyme, the ceramidase, or a combination thereof, comprises a carboxylesterase, a lipase, a lipoprotein lipase, an acylglycerol lipase, a hormone-sensitive lipase, a phospholipase A₁, a phospholipases A₂, a phosphatidylinositol deacylase, a phospholipase C, a phospholipase D, a phosphoinositide phospholipase C, a phosphatidate phosphatase, a lysophospholipase, a sterol esterase, a galactolipase, a sphingomyelin phosphodiesterase, a sphingomyelin phosphodiesterases D, a ceramidase, a wax-ester hydrolase, a fatty-acyl-ethyl-ester synthase, a retinyl-palmitate esterase, a 11-cis-retinyl-palmitate hydrolase, an all-trans-retinyl-palmitate hydrolase, a cutinase, an acyloxyacyl hydrolase, or a combination thereof.

5. The composition of claim 4, wherein the lipolytic enzyme, the ceramidase, or a combination thereof comprises: a carboxylesterase derived from Actinidia deliciosa, Aedes aegypti, Aeropyrum pernix, Alicyclobacillus acidocaldarius, Aphis gossypii, Arabidopsis thaliana, Archaeoglobus fulgidus, Aspergillus clavatus, Athalia rosae, Bacillus acidocaldarius, Bombyx mandarina, Bombyx mori, Bos taurus, Burkholderia gladioli, Caenorhabditis elegans, Canis familiaris, Cavia porcellus, Chloroflexus aurantiacus, Felis catus, Fervidobacterium nodosum, Helicoverpa armigera, Homo sapiens, Macaca fascicularis, Malus pumila, Mesocricetus auratus, Mus musculus, Musca domestica, Mycoplasma hyopneumoniae, Myxococcus xanthus, Neosartorya fischeri, Oryctolagus cuniculus, Paeonia suffruticosa, Pseudomonas aeruginosa, Rattus norvegicus, Rubrobacter xylanophilus, Spodoptera exigua, Spodoptera litura, Sulfolobus acidocaldarius, Sulfolobus shibatae, Sulfolobus solfataricus, Sus scrofa, Thermotoga maritime, Thermus thermophilus, Vaccinium corymbosum, Vibrio harveyi, Xenopsylla cheopis, Yarrowia lipolytica, or a combination thereof; a lipase derived from Acinetobacter, Aedes aegypti, Anguilla japonica, Antrodia cinnamomea, Arabidopsis rosette, Arabidopsis thaliana, Arxula adeninivorans, Aspergillus niger, Aspergillus oryzae, Aspergillus tamarii, Aureobasidium pullulans, Avena sativa, Bacillus lichenifonnis, Bacillus sphaericus, Bacillus stearothermophilus, Bacillus subtilis, Bacillus thermocatenulatus, Bacillus thermoleovorans, Bombyx mandarina, Bombyx mori, Bos Taurus, Brassica napus, Brassica rapa, Burkholderia cepacia, Caenorhabditis elegans, Candida albicans, Candida antarctica, Candida deformans, Candida parapsilosis, Candida rugosa, Candida thermophila, Canis domesticus, Chenopodium rubrum, Clostridium beijerinckii, Clostridium botulinum, Clostridium novyi, Danio rerio, Galactomyces geotrichum, Gallus gallus, Geobacillus, Gibberella zeae, Gossypium hirsutum, Homo sapiens, Kurtzmanomyces sp., Leishmania infantum, Lycopersicon esculentum L, Malassezia furfur, Methanosarcina acetivorans, Mus musculus, Mus spretus, Mycobacterium tuberculosis, Mycoplasma hyopneumoniae, Myxococcus xanthus, Neosartorya fischeri, Oryctolagus cuniculus, Oryza sativa, Penicillium cyclopium, Phlebotomus papatasi, Pseudomonas aeruginosa, Pseudomonas fluorescens, Pseudomonas fragi, Pseudomonas sp, Rattus norvegicus, Rhizomucor miehei, Rhizopus oryzae, Rhizopus stolonifer, Ricinus communis, Samia cynthia ricini, Schizosaccharomyces pombe, Serratia marcescens, Spermophilus tridecemlineatus, Staphylococcus simulans, Staphylococcus xylosus, Sulfolobus solfataricus, Sus scrofa, Thermomyces lanuginosus, Trichomonas vaginalis, Vibrio harveyi, Xenopus laevis, Yarrowia lipolytica, or a combination thereof; a lipoprotein lipase derived from Capra hircus, Danio rerio, Felis catus, Homo sapiens, Mesocricetus auratus, Mus musculus, Oncorhynchus mykiss, Pagrus major, Papio Anubis, Rattus norvegicus, Sparus aurata, Sus scrofa, Thunnus orientalis, or a combination thereof; an acylglycerol lipase derived from Bacillus sp., Danio rerio, Homo sapiens, Leishmania infantum, Mus musculus, Mycobacterium tuberculosis, Penicillium camembertii, Rattus norvegicus, Solanum tuberosum, or a combination thereof; a hormone sensitive lipase derived from Bos Taurus, Homo sapiens, Mus musculus, Rattus norvegicus, Spermophilus tridecemlineatus, Sus scrofa, Tetrahymena thermophila, or a combination thereof; a phospholipase A₁ derived from Arabidopsis, Aspergillus oryzae, Bos Taurus, Brassica rapa, Caenorhabditis elegans, Capsicum annuum, Danio rerio, Homo sapiens, Mus musculus, Nicotiana tabacum, Polistes annularis, Polybia paulista, Rattus norvegicus, Serratia sp., Vespula vulgaris, or a combination thereof; a phospholipase A₂ derived from Acanthaster planci, Adamsia carciniopado, Aedes aegypti, Aeropyrum pernix, Aipysurus eydouxii, Apis mellifera, Arabidopsis thaliana, Aspergillus nidulans, Austrelaps superbus, Bitis gabonica, Bos taurus, Bothriechis schlegelii, Bothrops jararacussu, BrachyDanio rerio, Bungarus caeruleus, Bungarus fasciatus, Canis familiaris, Cavia sp., Cerrophidion godmani, Chlamydomonas reinhardtii, Chrysophrys major, Crotalus viridis viridis, Daboia russellii, Danio rerio, Drosophila melanogaster, Echis carinatus, Echis ocellatus, Echis pyramidum leakeyi, Emericella nidulans, Equus caballus, Gallus gallus, Homo sapiens, Lapemis hardwickii, Laticauda semifasciata, Micrurus corallines, Mus musculus, Mytilus edulis, Naja kaouthia, Naja naja, Naja naja sputatrix, Nicotiana tabacum, Ophiophagus hannah, Ornithodoros parkeri, Oryctolagus cuniculus, Pagrus major, Patiria pectinifera, Polyandrocarpa misakiensis, Protobothrops mucrosquamatus, Rattus norvegicus, Sistrurus catenatus tergeminus, Trimeresurus borneensis, Trimeresurus flavoviridis, Trimeresurus gracilis, Trimeresurus gramineus, Trimeresurus okinavensis, Trimeresurus puniceus, Trimeresurus stejnegeri, Tuber borchii, Urticina crassicornis, Vipera russelli siamensis, Xenopus laevis, Xenopus tropicalis, or a combination thereof; a phospholipase C derived from Aedes aegypti, Aplysia californica, Arabidopsis thaliana, Asterina miniata, Bacillus cereus, Bacillus thuringiensis, Bos taurus, Caenorhabditis elegans, Chaetopterus pergamentaceus, Chlamydomonas reinhardtii, Coturnix japonica, Danio rerio, Dictyostelium discoideum, Drosophila melanogaster, Gallus gallus, Homarus americanus, Homo sapiens, Loligo pealei, Lytechinus pictus, Meleagris gallopavo, Misgurnus mizolepis, Mus musculus, Nicotiana tabacum, Oryza sativa, Oryzias latipes, Petunia inflate, Pichia stipitis, Pisum sativum, Plasmodium falciparum, Rattus norvegicus, Strongylocentrotus purpuratus, Sus scrofa, Torenia fournieri, Toxoplasma gondii, Watasenia scintillans, Xenopus laevis, Zea mays, or a combination thereof; a phospholipase D derived from Aedes aegypti, Arabidopsis thaliana, Arachis hypogaea, Bos taurus, Brassica oleracea, Caenorhabditis elegans, Cricetulus griseus, Cucumis melo var. inodorus, Cucumis sativus, Dictyostelium discoideum, Drosophila melanogaster, Emericella nidulans, Fragaria ananassa, Gossypium hirsutum, Homo sapiens, Lolium temulentum, Lycopersicon esculentum, Mus musculus, Oryza sativa, Papaver somniferum, Paralichthys olivaceus, Pichia stipitis, Pimpinella brachycarpa, Rattus norvegicus, Ricinus communis, Streptoverticillium cinnamoneum, Vigna unguiculata, Vitis vinifera, Zea mays, or a combination thereof; a phosphoinositide phospholipase C derived from Arabidopsis thaliana, Aspergillus clavatus, Aspergillus fumigatus, Brassica napus, Homo sapiens, Leishmania infantum, Mus musculus, Neosartorya fischeri, Physcomitrella patens, Pichia stipitis, Rattus norvegicus, Toxoplasma gondii, Trypanosoma brucei, Vigna unguiculata, Xenopus tropicalis, Zea mays, or a combination thereof; a phosphatidate phosphatase derived from Saccharomyces cerevisiae, or a combination thereof; a lysophospholipase derived from Aedes aegypti, Argas monolakensis, Aspergillus clavatus, Aspergillus fumigatus, Bos Taurus, Cavia porcellus, Clonorchis sinensis, Danio rerio, Dictyostelium discoideum, Emericella nidulans, Giardia lamblia, Homo sapiens, Monodelphis domestica, Mus musculus, Neosartorya fischeri, Pichia jadinii, Pichia stipitis, Rattus norvegicus, Schistosoma japonicum, Schizosaccharomyces pombe, Sclerotinia sclerotiorum, Xenopus tropicalis, or a combination thereof; a sterol esterase derived from Candida rugosa, Homo sapiens, Melanocarpus albomyces, Rattus norvegicus, or a combination thereof; a galactolipase derived from Homo sapiens, Solanum tuberosum, Vigna unguiculata, or a combination thereof; a sphingomyelin phosphodiesterase derived from Bacillus cereus, Homo sapiens, Pseudomonas sp., or a combination thereof; a ceramidase derived from Homo sapiens, Pseudomonas, or a combination thereof; a cutinase derived from Fusarium solani pisi, Monilinia fructicola, Pseudomonas putida, or a combination thereof; a retinyl palmitate esterase derived from Bos Taurus; or a combination thereof.

6. The composition of claim 5, wherein the lipolytic enzyme comprises: a thermophilic carboxylesterase derived from Aeropyrum pernix, Alicyclobacillus acidocaldarius, Archaeoglobus fulgidus, Bacillus acidocaldarius, Pseudomonas aeruginosa, Sulfolobus shibatae, Sulfolobus solfataricus, Thermotoga maritime, or a combination thereof; a themophilic lipase derived from Acinetobacter calcoaceticus, Acinetobacter sp., Bacillus sphaericus, Bacillus stearothermophilus, Bacillus thermocatenulatus, Bacillus thermoleovorans, Candida rugosa, Candida thermophila, GeoBacillus thermoleovorans Toshki, Pseudomonas fragi, Staphylococcus xylosus, Sulfolobus solfataricus, or a combination thereof; a psychrophilic lipase derived from Pseudomonas fluorescens; or a combination thereof; a thermophilic phospholipase A₂ derived from Aeropyrum pernix; a thermophilic phospholipase C derived from Bacillus cereus; or a combination thereof.

7. The composition of claim 3, wherein the phosphoric triester hydrolase comprises an aryldialkylphosphatase, a diisopropyl-fluorophosphatase, or a combination thereof.

8. The composition of claim 7, wherein the aryldialkylphosphatase comprises an organophosphorus hydrolase, a human paraoxonase, an animal carboxylase, or a combination thereof; wherein the diisopropyl-fluorophosphatase comprises an organophosphorus acid anhydrolase, a squid-type DFPase, a Mazur-type DFPase, or a combination thereof; or a combination thereof of the forgoing.

9. The composition of claim 8, wherein the organophosphorus hydrolase comprises an Agrobacterium radiobacter P230 organophosphate hydrolase, a Flavobacterium balustinum parathion hydrolase, a Pseudomonas diminuta phosphotriesterase, a Flavobacterium sp opd gene product, a Flavobacterium sp. parathion hydrolase opd gene product, or a combination thereof; wherein the animal carboxylase comprises an insect carboxylase; or a combination thereof; wherein the organophosphorus acid anhydrolase comprises an Altermonas organophosphorus acid anhydrolase, a prolidase, or a combination thereof; wherein the squid-type DFPase comprises a Loligo vulgaris DFPase, a Loligo pealei DFPase, a Loligo opalescens DFPase, or a combination thereof; wherein the Mazur-type DFPase comprises a mouse liver DFPase, a hog kidney DFPase, a Bacillus stearothermophilus strain OT DFPase, an Escherichia coli DFPase, or a combination thereof; or a combination thereof the forgoing.

10. The composition of claim 8, wherein the insect carboxylase comprises a Podia interpunctella carboxylase, Chrysomya putoria carboxylase, Lucilia cuprina carboxylase, Musca domestica carboxylase, or a combination thereof; wherein the Altermonas organophosphorus acid anhydrolase comprises an Alteromonas sp JD6.5 organophosphorus acid anhydrolase, an Alteromonas haloplanktis organophosphorus acid anhydrolase, an Altermonas undina organophosphorus acid anhydrolase, or a combination thereof; wherein the prolidase comprises a human prolidase, a Mus musculus prolidase, a Lactobacillus helveticus prolidase, an Escherichia coli prolidase, an Escherichia coli aminopeptidase P, or a combination thereof; wherein the phosphoric triester hydrolase comprises a Plesiomonas sp. strain M6 mpd gene product, a Xanthomonas sp. phosphoric triester hydrolase, a Tetrahymena phosphoric triester hydrolase, or a combination thereof; or a combination thereof the forgoing.

11. The composition of claim 3, wherein the sulfuric ester hydrolase comprises an arylsulfatase.

12. The composition of claim 1, wherein the peptidase comprises a trypsin, a chymotrypsin, or a combination thereof.

13. The composition of claim 1, wherein the antibiological enzyme comprises a lysozyme, a lysostaphin, a libiase, a lysyl endopeptidase, a mutanolysin, a cellulase, a chitinase, an α-agarase, an β-agarase, a N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyl)asparagine amidase, a mannosyl-glycoprotein endo-β-N-acetylglucosaminidase, a l-carrageenase, a κ-carrageenase, a λ-carrageenase, an α-neoagaro-oligosaccharide hydrolase, an endolysin, an autolysin, a mannoprotein protease, a glucanase, a mannase, a zymolase, a lyticase, a lipolytic enzyme, a peroxidase, or a combination thereof.

14. The composition of claim 1, wherein the antibiological peptidic agent comprises SEQ ID No. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or a combination thereof.

15. The composition of claim 1, wherein the antibiological peptidic agent comprises a plurality of antibiological peptidic agents.

16. The composition of claim 1, wherein the active enzyme comprises a mesophilic enzyme, a psychrophilic enzyme, a thermophilic enzyme, a halophilic enzyme, or a combination thereof.

17. The composition of claim 1, wherein the active enzyme, the antibiological peptidic agent, or a combination thereof, comprises an immobilization carrier.

18. The composition of claim 1, wherein the active enzyme, the antibiological peptidic agent, or a combination thereof, comprises a purified active enzyme, a purified antibiological peptidic agent, or a combination thereof.

19. The composition of claim 1, wherein the active enzyme, the antibiological peptidic agent, or a combination thereof, comprises a particulate material.

20. The composition of claim 19, wherein the active enzyme, the antibiological peptidic agent, or a combination thereof, comprises a cell-based particulate material.

21. The composition of claim 20, wherein the cell-based particulate material comprises a whole cell particulate material or a cell fragment particulate material.

22. The composition of claim 19, wherein the average wet molecular weight or dry molecular weight of a primary particle of the particulate material is about 50 kDa to about 1.5×10¹⁴ kDa.

23. The composition of claim 19, wherein an average active enzyme content, an average antibiological peptidic agent content, or a combination thereof, per primary particle of the particulate material is about 0.01% to about 100%.

24. The composition of claim 1, wherein the active enzyme, the antibiological peptidic agent, or a combination thereof, is attenuated, sterilized, or a combination thereof.

25. The composition of claim 1, wherein the active enzyme, the antibiological peptidic agent, or a combination thereof, comprises about 0.01% to about 80% of the composition by weight or volume.

26. The composition of claim 1, wherein the active enzyme, the antibiological peptidic agent, or a combination thereof, is microencapsulated.

27. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, is about 5 um to about 5000 um thick upon a surface.

28. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a paint.

29. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a clear coating.

30. The composition of claim 29, wherein the clear coating comprises a lacquer, a varnish, a shellac, a stain, a water repellent coating, or a combination thereof.

31. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a multicoat system.

32. The composition of claim 31, wherein the multicoat system comprises 2 to 10 layers.

33. The composition of claim 31, wherein a plurality of layers of the multicoat system comprise the active enzyme.

34. The composition of claim 31, wherein the multicoat system comprises a sealer, a water repellent, a primer, an undercoat, a topcoat, or a combination thereof.

35. The composition of claim 34, wherein the topcoat comprises the active enzyme.

36. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a coating that is capable of film formation.

37. The composition of claim 36, wherein film formation occurs between about −10° C. to about 40° C.

38. The composition of claim 36, wherein film formation occurs at baking conditions.

39. The composition of claim 36, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a volatile component and a non-volatile component, and wherein film formation occurs by loss of part of the volatile component.

40. The composition of claim 36, wherein film formation occurs by cross-linking of a binder.

41. The composition of claim 36, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, produces a self-cleaning film.

42. The composition of claim 36, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, produces a temporary film.

43. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a non-film forming coating.

44. The composition of claim 43, wherein the non-film forming coating comprises a non-film formation binder.

45. The composition of claim 43, wherein the non-film forming coating comprises a coating component in a concentration that is insufficient to produce a solid film.

46. The composition of claim 1, wherein the architectural coating comprises an architectural wood coating, an architectural masonry coating, an architectural artist's coating, an architectural plastic coating, an architectural metal coating, or a combination thereof.

47. The composition of claim 1, wherein the architectural coating has a pot life of at least 12 months at about −10° C. to about 40° C.

48. The composition of claim 1, wherein the composition comprises an automotive coating, a can coating, a sealant coating, or a combination thereof.

49. The composition of claim 1, wherein the composition comprises a chemical agent resistant coating, a camouflage coating, a pipeline coating, a traffic marker coating, an aircraft coating, a nuclear power plant coating, or a combination thereof.

50. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a coating for a plastic surface.

51. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a water-borne coating.

52. The composition of claim 51, wherein the water-borne coating comprises a latex coating.

53. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a solvent-borne coating.

54. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, has a low-shear viscosity of about 100 P to about 3000 P, has a medium-shear viscosity of about 84 Ku and about 140 Ku, has a high-shear viscosity of about 0.5 P to about 2.5 P, or a combination thereof.

55. The composition of claim 1, wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, comprises a binder, a liquid component, a colorant, an additive, or a combination thereof.

56. The composition of claim 55, wherein the binder comprises a thermoplastic binder, a thermosetting binder, or a combination thereof.

57. The composition of claim 55, wherein the binder comprises an oil-based binder, a polyester resin, a modified cellulose, a polyamide, an amino resin, a urethane binder, a phenolic resin, an epoxy resin, a polyhydroxyether binder, an acrylic resin, a polyvinyl binder, a rubber resin, a bituminous binder, a polysulfide binder, a silicone binder, an organic binder, or a combination thereof.

58. The composition of claim 57, wherein the oil-based binder comprises an oil, an alkyd, an oleoresinous binder, a fatty acid epoxide ester, or a combination thereof; wherein the polyester resin comprises a hydroxy-terminated polyester, a carboxylic acid-terminated polyester, or a combination thereof; wherein the modified cellulose comprises a cellulose ester, a nitrocellulose, or a combination thereof; wherein the epoxy resin comprises a cycloaliphatic epoxy binder; wherein the rubber resin comprises a chlorinated rubber resin, a synthetic rubber resin, or a combination thereof; or a combination thereof the forgoing.

59. The composition of claim 55, wherein the liquid component comprises a solvent, a thinner, a diluent, a plasticizer, or a combination thereof.

60. The composition of claim 55, wherein the liquid component comprises a liquid organic compound, an inorganic compound, water, or a combination thereof.

61. The composition of claim 60, wherein the liquid organic compound comprises a hydrocarbon, an oxygenated compound, a chlorinated hydrocarbon, a nitrated hydrocarbon, a miscellaneous organic liquid, a plasticizer, or a combination thereof; wherein the inorganic compound comprises ammonia, hydrogen cyanide, hydrogen fluoride, hydrogen cyanide, sulfur dioxide, or a combination thereof; wherein the water comprises methanol, ethanol, propanol, isopropyl alcohol, tert-butanol, ethylene glycol, methyl glycol, ethyl glycol, propyl glycol, butyl glycol, ethyl diglycol, methoxypropanol, methyldipropylene glycol, dioxane, tetrahydrorfuran, acetone, diacetone alcohol, dimethylformamide, dimethyl sulfoxide, ethylbenzene, tetrachloroethylene, p-xylene, toluene, diisobutyl ketone, tricholorethylene, trimethylcyclohexanol, cyclohexyl acetate, dibutyl ether, trimethylcyclohexanone, 1,1,1-tricholoroethane, hexane, hexanol, isobutyl acetate, butyl acetate, isophorone, nitropropane, butyl glycol acetate, 2-nitropropane, methylene chloride, methyl isobutyl ketone, cyclohexanone, isopropyl acetate, methylbenzyl alcohol, cyclohexanol, nitroethane, methyl tert-butyl ether, ethyl acetate, diethyl ether, butanol, butyl glycolate, isobutanol, 2-butanol, propylene carbonate, ethyl glycol acetate, methyl acetate, methyl ethyl ketone, or a combination thereof; or a combination thereof the forgoing.

62. The composition of claim 61, wherein the hydrocarbon comprises an aliphatic hydrocarbon, a cycloaliphatic hydrocarbon, a terpene, an aromatic hydrocarbon, or a combination thereof; wherein the oxygenated compound comprises an alcohol, an ester, a glycol ether, a ketone, an ether, or a combination thereof; or a combination thereof the forgoing.

63. The composition of claim 61, wherein the hydrocarbon comprises a petroleum ether, pentane, hexane, heptane, isododecane, a kerosene, a mineral spirit, a VMP naphtha, cyclohexane, methylcyclohexane, ethylcyclohexane, tetrahydronaphthalene, decahydronaphthalene, wood terpentine oil, pine oil, α-pinene, β-pinene, dipentene, D-limonene, benzene, toluene, ethylbenzene, xylene, cumene, a type I high flash aromatic naphtha, a type II high flash aromatic naphtha, mesitylene, pseudocumene, cymol, styrene, or a combination thereof; wherein the oxygenated compound comprises methanol, ethanol, propanol, isopropanol, 1-butanol, isobutanol, 2-butanol, tert-butanol, amyl alcohol, isoamyl alcohol, hexanol, methylisobutylcarbinol, 2-ethylbutanol, isooctyl alcohol, 2-ethylhexanol, isodecanol, cylcohexanol, methylcyclohexanol, trimethylcyclohexanol, benzyl alcohol, methylbenzyl alcohol, furfuryl alcohol, tetrahydrofurfuryl alcohol, diacetone alcohol, trimethylcyclohexanol, methyl formate, ethyl formate, butyl formate, isobutyl formate, methyl acetate, ethyl acetate, propyl acetate, isopropyl acetate, butyl acetate, isobutyl acetate, sec-butyl acetate, amyl acetate, isoamyl acetate, hexyl acetate, cyclohexyl acetate, benzyl acetate, methyl glycol acetate, ethyl glycol acetate, butyl glycol acetate, ethyl diglycol acetate, butyl diglycol acetate, 1-methoxypropyl acetate, ethoxypropyl acetate, 3-methoxybutyl acetate, ethyl 3-ethoxypropionate, isobutyl isobutyrate, ethyl lactate, butyl lactate, butyl glycolate, dimethyl adipate, glutarate, succinate, ethylene carbonate, propylene carbonate, butyrolactone, methyl glycol, ethyl glycol, propyl glycol, isopropyl glycol, butyl glycol, methyl diglycol, ethyl diglycol, butyl diglycol, ethyl triglycol, butyl triglycol, diethylene glycol dimethyl ether, methoxypropanol, isobutoxypropanol, isobutyl glycol, propylene glycol monoethyl ether, 1-isopropoxy-2-propanol, propylene glycol mono-n-propyl ether, propylene glycol n-butyl ether, methyl dipropylene glycol, methoxybutanol, acetone, methyl ethyl ketone, methyl propyl ketone, methyl isopropyl ketone, methyl butyl ketone, methyl isobutyl ketone, methyl amyl ketone, methyl isoamyl ketone, diethyl ketone, ethyl amyl ketone, dipropyl ketone, diisopropyl ketone, cyclohexanone, methylcylcohexanone, trimethylcyclohexanone, mesityl oxide, diisobutyl ketone, isophorone, diethyl ether, diisopropyl ether, dibutyl ether, di-sec-butyl ether, methyl tert-butyl ether, tetrahydrofuran, 1,4-dioxane, metadioxane, or a combination thereof; wherein the chlorinated hydrocarbon comprises methylene chloride, trichloromethane, tetrachloromethane, ethyl chloride, isopropyl chloride, 1,2-dichloroethane, 1,1,1-trichloroethane, trichloroethylene, 1,1,2,2-tetrachlorethane, 1,2-dichloroethylene, perchloroethylene, 1,2-dichloropropane, chlorobenzene, or a combination thereof; wherein the nitrated hydrocarbon comprises a nitroparaffin, N-methyl-2-pyrrolidone, or a combination thereof; wherein the miscellaneous organic liquid comprises carbon dioxide; acetic acid, methylal, dimethylacetal, N,N-dimethylformamide, N,N-dimethylacetamide, dimethylsulfoxide, tetramethylene suflone, carbon disulfide, 2-nitropropane, N-methylpyrrolidone, hexamethylphosphoric triamide, 1,3-dimethyl-2-imidazolidinone, or a combination thereof; wherein the plasticizer comprises di(2-ethylhexyl) azelate; di(butyl) sebacate; di(2-ethylhexyl) phthalate; di(isononyl) phthalate; dibutyl phthalate; butyl benzyl phthalate; di(isooctyl) phthalate; di(idodecyl) phthalate; tris(2-ethylhexyl) trimellitate; tris(isononyl) trimellitate; di(2-ethylhexyl) adipate; di(isononyl) adipate; acetyl tri-n-butyl citrate; an epoxy modified soybean oil; 2-ethylhexyl epoxytallate; isodecyl diphenyl phosphate; tricresyl phosphate; isodecyl diphenyl phosphate; tri-2-ethylhexyl phosphate; an adipic acid polyester; an azelaic acid polyester; a bisphenoxyethylformal, or a combination thereof; or a combination thereof the forgoing.

64. The composition of claim 63, wherein the colorant comprises a pigment, a dye, or a combination thereof.

65. The composition of claim 64, wherein the active enzyme comprises a particulate material comprising about 0.000001% to about 100% of the pigment.

66. The composition of claim 64, wherein the pigment volume concentration of wherein the architectural coating, the automotive coating, the can coating, the sealant coating, the chemical agent resistant coating, the camouflage coating, the pipeline coating, the traffic marker coating, the aircraft coating, the nuclear power plant coating, or a combination thereof, is about 20% to about 70%.

67. The composition of claim 64, wherein the pigment comprises a corrosion resistance pigment, a camouflage pigment, a color property pigment, an extender pigment, or a combination thereof.

68. The composition of claim 67, wherein the corrosion resistance pigment comprises aluminum flake, aluminum triphosphate, aluminum zinc phosphate, ammonium chromate, barium borosilicate, barium chromate, barium metaborate, basic calcium zinc molybdate, basic carbonate white lead, basic lead silicate, basic lead silicochromate, basic lead silicosulfate, basic zinc molybdate, basic zinc molybdate-phosphate, basic zinc molybdenum phosphate, basic zinc phosphate hydrate, bronze flake, calcium barium phosphosilicate, calcium borosilicate, calcium chromate, calcium plumbate, calcium strontium phosphosilicate, calcium strontium zinc phosphosilicate, dibasic lead phosphite, lead chromosilicate, lead cyanamide, lead suboxide, lead sulfate, mica, micaceous iron oxide, red lead, steel flake, strontium borosilicate, strontium chromate, tribasic lead phophosilicate, zinc borate, zinc borosilicate, zinc chromate, zinc dust, zinc hydroxy phosphite, zinc molybdate, zinc oxide, zinc phosphate, zinc potassium chromate, zinc silicophosphate hydrate, zinc tetraoxylchromate, or a combination thereof; wherein the camouflage pigment comprises an anthraquinone black, a chromium oxide green, the active enzyme comprising a particulate material, or a combination thereof; wherein the color property pigment comprises a black pigment, a brown pigment, a white pigment, a pearlescent pigment, a violet pigment, a blue pigment, a green pigment, a yellow pigment, an orange pigment, a red pigment, a metallic pigment, the active enzyme comprising a particulate material, or a combination thereof; wherein the extender pigment comprises a barium sulphate, a calcium carbonate, a kaolin, a calcium sulphate, a silicate, a silica, an alumina trihydrate, an active enzyme comprising a particulate material, or a combination thereof; or a combination thereof the forgoing.

69. The composition of claim 67, wherein the color property pigment comprises aniline black; anthraquinone black; carbon black; copper carbonate; graphite; iron oxide; micaceous iron oxide; manganese dioxide, azo condensation, metal complex brown; antimony oxide; basic lead carbonate; lithopone; titanium dioxide; white lead; zinc oxide; zinc sulphide; titanium dioxide and ferric oxide covered mica, bismuth oxychloride crystal, dioxazine violet, carbazole Blue; cobalt blue; indanthrone; phthalocyanine blue; Prussian blue; ultramarine; chrome green; hydrated chromium oxide; phthalocyanine green; anthrapyrimidine; arylamide yellow; barium chromate; benzimidazolone yellow; bismuth vanadate; cadmium sulfide yellow; complex inorganic color; diarylide yellow; disazo condensation; flavanthrone; isoindoline; isoindolinone; lead chromate; nickel azo yellow; organic metal complex; yellow iron oxide; zinc chromate; perinone orange; pyrazolone orange; anthraquinone; benzimidazolone; BON arylamide; cadmium red; cadmium selenide; chrome red; dibromanthrone; diketopyrrolo-pyrrole; lead molybdate; perylene; pyranthrone; quinacridone; quinophthalone; red iron oxide; red lead; toluidine red; tonor; β-naphthol red; aluminum flake; aluminum non-leafing, gold bronze flake, zinc dust, stainless steel flake, nickel flake, nickel powder, barium ferrite; borosilicate; burnt sienna; burnt umber; calcium ferrite; cerium; chrome orange; chrome yellow; chromium phosphate; cobalt-containing iron oxide; fast chrome green; gold bronze powder; luminescent; magnetic; molybdate orange; molybdate red; oxazine; oxysulfide; polycyclic; raw sienna; surface modified pigment; thiazine; thioindigo; transparent cobalt blue; transparent cobalt green; transparent iron blue; transparent zinc oxide; triarylcarbonium; zinc cyanamide; zinc ferrite; or a combination thereof.

70. The composition of claim 55, wherein the additive comprises 0.000001% to 20.0% by weight, of the architectural coating, the automotive coating, the can coating, the sealant coating, the chemical agent resistant coating, the camouflage coating, the pipeline coating, the traffic marker coating, the aircraft coating, the nuclear power plant coating, or a combination thereof.

71. The composition of claim 55, wherein the additive comprises an accelerator, an adhesion promoter, an antifoamer, anti-insect additive, an antioxidant, an antiskinning agent, a buffer, a catalyst, a coalescing agent, a corrosion inhibitor, a defoamer, a dehydrator, a dispersant, a drier, electrical additive, an emulsifier, a filler, a flame/fire retardant, a flatting agent, a flow control agent, a gloss aid, a leveling agent, a marproofing agent, a preservative, a silicone additive, a slip agent, a surfactant, a light stabilizer, a rheological control agent, a wetting additive, a cryopreservative, a xeroprotectant, a pH indicator, or a combination thereof.

72. The composition of claim 71, wherein the preservative comprises an in-can preservative, an in-film preservative, or a combination thereof.

73. The composition of claim 72, wherein the preservative comprises a biocide, a biostatic, or a combination thereof.

74. The composition of claim 73, wherein the biocide, the biostatic, or a combination thereof comprises an algaecide, an algaestatic, a bactericide, a bacteristatic, a fungicide, a fungistatic, a germicide, a germistatic, a herbicide, a herbistatic, a microbiocide, a microbiostatic, a mildewcide, a mildewstatic, a molluskicide, a molluskistatic, a slimicide, a slimistatic, a viricide, a viristatic, or a combination thereof.

75. The composition of claim 71, wherein the preservative comprises 1-(3-chloroallyl)-3,5,7-triaza-1-azoniaadamantane chloride; 1,2-benzisothiazoline-3-one; 1,2-dibromo-2,4-dicyanobutane; 1,3-bis(hydroxymethyl)-5,5-dimethylhydantoin; 1-methyl-3,5,7-triaza-1-azonia-adamantane chloride; 2-bromo-2-nitropropane-1,3-diol; 2-(4-thiazolyl)benzimidazole; 2-(hydroxymethyl)-amino-2-methyl-1-propanol; 2(hydroxymethyl)-aminoethanol; 2,2-dibromo-3-nitrilopropionamide; 2,4,5,6-tetrachloro-isophthalonitrile; 2-mercaptobenzo-thiazole; 2-methyl-4-isothiazolin-3-one; 2-n-octyl-4-isothiazoline-3-one; 3-iodo-2-propynl N-butyl carbamate; 4,5-dichloro-2-N-octyl-3(2H)-isothiazolone; 4,4-dimethyloxazolidine; 5-chloro-2-methyl-4-isothiazolin-3-one; 5-hydroxy-methyl-1-aza-3,7-dioxabicylco (3.3.0.) octane; 6-acetoxy-2,4-dimethyl-1,3-dioxane; 7-ethyl bicyclooxazolidine; a combination of 1,2-benzisothiazoline-3-one and hexahydro-1,3,5-tris(2-hydroxyethyl)-s-triazine; a combination of 1,2-benzisothiazoline-3-one and zinc pyrithione; a combination of 2-(thiocyanomethyl-thio)benzothiozole and methylene bis(thiocyanate); a combination of 4-(2-nitrobutyl)-morpholine and 4,4′-(2-ethylnitrotrimethylene) dimorpholine; a combination of 4,4-dimethyl-oxazolidine and 3,4,4-trimethyloxazolidine; a combination of 5-chloro-2-methyl-4-isothiazolin-3-one and 2-methyl-4-isothiazolin-3-one; a combination of carbendazim and 3-iodo-2-propynl N-butyl carbamate; a combination of carbendazim, 3-iodo-2-propynl N-butyl carbamate and diuron; a combination of chlorothalonil and 3-iodo-2-propynl N-butyl carbamate; a combination of chlorothalonil and a triazine compound; a combination of tributyltin benzoate and alkylamine hydrochlorides; a combination of zinc-dimethyldithiocarbamate and zinc 2-mercaptobenzothiazole; a copper soap; a metal soap; a mercury soap; a mixture of bicyclic oxazolidines; a tin soap; an alkylamine hydrochloride; an amine reaction product; barium metaborate; butyl parahydroxybenzoate; carbendazim; copper(II) 8-quinolinolate; diiodomethyl-p-tolysulfone; dithio-2, 2-bis(benzmethylamide); diuron; ethyl parahydroxybenzoate; glutaraldehyde; hexahydro-1,3,5-triethyl-s-triazine; hexahydro-1,3,5-tris(2-hydroxyethyl)-s-triazine; hydroxymethyl-5,5-dimethylhydantoin; methyl parahydroxybenzoate; N-butyl-1,2-benzisothiazolin-3-one; N-(trichloromethylthio) phthalimide; N-cyclopropyl-N-(1-dimethylethyl)-6-(methylthio)-1,3,5-triazine-2,4-diamine; N-trichloromethyl-thio-4-cyclohexene-1,2-dicarboximide; p-chloro-m-cresol; phenoxyethanol; phenylmercuric acetate; poly(hexamethylene biguanide) hydrochloride; potassium dimethyldithiocarbamate; potassium N-hydroxy-methyl-N-methyl-dithiocarbamate; propyl parahydroxybenzoate; sodium 2-pyridinethiol-1-oxide; tetra-hydro-3,5-di-methyl-2H-1,3,5-thiadiazine-2-thione; tributyltin benzoate; tributyltin oxide; tributyltin salicylate; zinc pyrithione; sodium pyrithione; copper pyrithione; zinc oxide; a zinc soap; or a combination thereof.

76. The composition of claim 1, wherein the elastomer comprises a thermoplastic elastomer, a melt processable rubber, a synthetic rubber, a natural rubber, a propylene oxide elastomer, an ethylene-isoprene elastomer, an ethylene-vinyl acetate elastomer, a non-polymeric elastomer, or a combination thereof.

77. The composition of claim 76, wherein the thermoplastic elastomer comprises an elastomeric polyolefin, a thermoplastic vulcanizate, a styrenic thermoplastic elastomer, a styrene butadiene rubber, a polyurethane elastomer, a thermoplastic copolyester elastomer, a polyamide, or a combination thereof; wherein the synthetic rubber comprises a nitrile butadiene rubber, a butadiene rubber, a butyl rubber, a chlorinated/chlorosulfonated polyethylene, an epichlorohydrin, an ethylene propylene copolymer, a fluoroelastomer, a polyacrylate rubber, a poly(ethylene acrylic), a polychloroprene, a polyisoprene, a polysulfide rubber, a silicone rubber, or a combination thereof; wherein the non-polymeric elastomer comprises a vulcanized oil; or a combination thereof.

78. The composition of claim 1, wherein the composition comprises an adhesive, a sealant, or a combination thereof.

79. The composition of claim 78, wherein the adhesive, the sealant, or a combination thereof; comprises an acrylic adhesive, an acrylic acid diester adhesive, a butyl rubber adhesive, a carbohydrate adhesive, a cellulosic adhesive, a cyanoacrylate adhesive, a cyanate ester adhesive, an epoxy adhesive, a melamine formaldehyde adhesive, a natural rubber adhesive, a neoprene rubber adhesive, a nitrile rubber adhesive, a phenolic adhesive, a phenoxy adhesive, a polyamide adhesive, a polybenzimidazole adhesive, a polyethylene adhesive, a polyester adhesive, a polyisobutylene adhesive, a polysulfide adhesive, a polyurethane adhesive, a polyvinyl acetal adhesive, a polyvinyl acetate adhesive, a polyvinyl alcohol adhesive, a protein adhesive, a reclaimed rubber adhesive, a resorcinol adhesive, a silicone adhesive, a styrene butadiene adhesive, an urea formaldehyde adhesive, a vinyl vinylidene adhesive, a non-polymeric adhesive, or a combination thereof.

80. The composition of claim 79, wherein the non-polymeric adhesive comprises a mucilage adhesive.

81. The composition of claim 1, wherein the elastomer; the adhesive; the sealant, or a combination thereof, comprises a polymeric material additive.

82. The composition of claim 81, wherein the polymeric material additive comprises a curing agent, a cross-linking agent, an inhibitor, a nucleating agent, a plasticizer, a lubricant, a mold release agent, a slip agent, a diluent, a dispersant, a thickening agent, a thixotropic, a thinner, an anti-blocking agent, an antistatic agent, a flame retarder, a colorant, an antifogging agent, an odorant, a blowing agent, a surfactant, a defoamer, an anti-aging additive, a degrading agent, an anti-microbial agent, an adhesion promoter, an impact modifier, a low-profile additive, a filler, a pH indicator, or a combination thereof.

83. The composition of claim 82, wherein the anti-microbial agent comprises a biocide, a biostatic, or a combination thereof.

84. The composition of claim 1, wherein the antibiological peptidic agent, the antibiological enzyme, or a combination thereof comprises a biocide, a biostatic, or a combination thereof.

85. The composition of claim 1, wherein the composition is stored in a multi-pack container.

86. The composition of claim 85, wherein about 0.000001% to about 100% of the active enzyme, the antibiological agent, or a combination thereof, is stored in a container of the multi-pack composition, and at least one composition component is stored in another container of the multi-pack.

87. A coating composition, comprising an architectural coating comprising an active enzyme, an antibiological peptidic agent, or a combination thereof, wherein the active enzyme comprises an esterase, a petroleum lipolytic enzyme, a ceramidase, a peptidase, an antibiological enzyme, or a combination thereof.

88. A multi-pack coating composition, comprising a plurality of containers, wherein at least one container comprises an active enzyme, an antibiological peptidic agent, or a combination thereof; wherein the active enzyme comprises an esterase, a petroleum lipolytic enzyme, a ceramidase, a peptidase, an antibiological enzyme, or a combination thereof; and wherein the coating comprises an architectural wood coating, an architectural masonry coating, an architectural artist coating, an automotive coating, a can coating, a sealant coating, a chemical agent resistant coating, a camouflage coating, a pipeline coating, a traffic marker coating, an aircraft coating, a nuclear power plant coating, or a combination thereof.

89. An elastomer composition, comprising an elastomer and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

90. A filler composition, comprising a filler and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

91. An adhesive composition, comprising an adhesive and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

92. A sealant composition, comprising a sealant and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

93. A textile finish composition, comprising a textile finish and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

94. A wax composition, comprising a wax and an active enzyme, an antibiological peptidic agent, or a combination thereof; and wherein the active enzyme comprises an esterase, a petroleum lipolytic enzyme, a ceramidase, a peptidase, an antibiological enzyme, or a combination thereof.

95. A method of preparing a bioactive surface treatment, a bioactive filler, or a combination thereof, comprising the steps of: obtaining an active enzyme, an antibiological peptidic agent, or a combination thereof; wherein the active enzyme comprises an esterase, a petroleum lipolytic enzyme, a ceramidase, a peptidase, an antibiological enzyme, or a combination thereof; and admixing at least one component of a surface treatment, a filler, or a combination thereof, with the active enzyme, the antibiological peptidic agent, or a combination thereof; and then admixing any additional component of a surface treatment, a filler, or a combination thereof to complete the surface treatment, the filler, or a combination thereof.

96. A method of preparing a bioactive surface treatment, a bioactive filler, or a combination thereof, comprising the steps of: obtaining an active enzyme, an antibiological peptidic agent, or a combination thereof; wherein the active enzyme comprises an esterase, a petroleum lipolytic enzyme, a ceramidase, a peptidase, an antibiological enzyme, or a combination thereof; and admixing a surface treatment, a filler, or a combination thereof, with the active enzyme, the antibiological peptidic agent, or a combination thereof.

97. A method of reducing the concentration of a chemical on a surface, comprising the steps of: applying a surface treatment to the surface, wherein the surface treatment comprises an active enzyme, and wherein the active enzyme comprises an esterase, a petroleum lipolytic enzyme, a ceramidase, a peptidase, an antibiological enzyme, or a combination thereof; and contacting the surface with a chemical, wherein the chemical comprises a substrate of the active enzyme; and wherein the substrate comprises an ester linkage, a peptide linkage, a lipid, a cell wall component, a cell membrane component, or a combination thereof.

98. The method of claim 97, wherein the surface treatment comprises an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, or a combination thereof.

99. The method of claim 97, wherein the substrate is a component of a living cell, a virus, or a combination thereof, and wherein the active enzyme produces a biocidal activity, a biostatic activity, or a combination thereof upon contact with the substrate.

100. A method of cleaning a surface contaminated with a chemical, comprising the steps of: contacting a surface contaminated with a chemical with a coating comprising an active enzyme, wherein the active enzyme comprises an esterase, a petroleum lipolytic enzyme, a ceramidase, a peptidase, an antibiological enzyme, or a combination thereof, wherein the chemical comprises a substrate of the active enzyme; and wherein the substrate comprises an ester linkage, a peptide linkage, a lipid, a cell wall component, a cell membrane component, or a combination thereof.

101. A method of reducing the concentration of a chemical on a surface, comprising the steps of: applying a coating to the surface, wherein the coating comprises an architectural wood coating, an architectural masonry coating, an architectural artist coating, an automotive coating, a can coating, a sealant coating, a camouflage coating, a pipeline coating, a traffic marker coating, an aircraft coating, a nuclear power plant coating, or a combination thereof, and wherein the coating comprises an active enzyme, wherein the active enzyme comprises an esterase, a petroleum lipolytic enzyme, a ceramidase, a peptidase, an antibiological enzyme, or a combination thereof, and contacting the surface with a chemical, wherein the chemical comprises a substrate of the active enzyme; and wherein the chemical comprises an ester linkage, a peptide linkage, a lipid, a cell wall component, a cell membrane component, or a combination thereof.

102. The method of claim 101, wherein the step of applying to the surface a coating occurs prior to contacting the surface with the chemical.

103. The method of claim 101, wherein the surface is located on a stove, a sink, a drain pipe, a counter top, a floor, a wall, a cabinet, an appliance, or a combination thereof.

104. The method of claim 101, wherein the coating is formulated as an interior coating.

105. The method of claim 101, further comprising the step of: applying a cleaning material to the surface, and removing the chemical, a product of the reaction of the chemical catalyzed by the active enzyme, or a combination thereof.

106. The method of claim 105, wherein the cleaning material comprises a cleaning solution, a cleaning devise, or a combination thereof.

107. A method of cleaning a surface contaminated with a chemical, comprising the steps of: obtaining a surface treatment comprising an active enzyme; and contacting a surface contaminated with a chemical with the surface treatment comprising an active enzyme, wherein the active enzyme comprises an esterase, a petroleum lipolytic enzyme, a ceramidase, a peptidase, an antibiological enzyme, or a combination thereof, wherein the chemical comprises a substrate of the active enzyme; and wherein the chemical comprises an ester linkage, a peptide linkage, a lipid, a cell wall component, a cell membrane component, or a combination thereof.

108. A kit having component parts capable of being assembled comprising a container comprising an active enzyme, an antibiological peptidic agent, or a combination thereof, and a container comprising at least one component of an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; wherein the active enzyme comprises an esterase, a petroleum lipolytic enzyme, a ceramidase, a peptidase, an antibiological enzyme, or a combination thereof.

109. An article of manufacture, comprising an architectural coating, an automotive coating, a can coating, a sealant coating, a chemical agent resistant coating, a camouflage coating, a pipeline coating, a traffic marker coating, an aircraft coating, a nuclear power plant coating; an elastomer; an adhesive; a sealant, a wax, a textile finish, a filler, or a combination thereof; wherein the article of manufacture comprises an active enzyme, an antibiological peptidic agent, or a combination thereof, and wherein the active enzyme comprises an esterase, a petroleum lipolytic enzyme, a ceramidase, a peptidase, an antibiological enzyme, or a combination thereof.


SEQUENCE LISTING (199 sequences)

SEQ ID NO: 1 - protein, 6 aa, Artificial Sequence
  Synthesized
XXXXRF

SEQ ID NO: 2 - protein, 6 aa, Artificial Sequence
  Synthesized
XXXXFH

SEQ ID NO: 3 - protein, 6 aa, Artificial Sequence
  Synthesized
XXXXKF

SEQ ID NO: 4 - protein, 6 aa, Artificial Sequence
  Synthesized
XXXXQR

SEQ ID NO: 5 - protein, 6 aa, Artificial Sequence
  Synthesized
XXXXRM

SEQ ID NO: 6 - protein, 6 aa, Artificial Sequence
  Synthesized
XXXXHM

SEQ ID NO: 7 - protein, 6 aa, Artificial Sequence
  Synthesized
XXXXKL

SEQ ID NO: 8 - protein, 6 aa, Artificial Sequence
  Synthesized
XXXXRL

SEQ ID NO: 9 - protein, 6 aa, Artificial Sequence
  Synthesized
XXXLRF

SEQ ID NO: 10 - protein, 6 aa, Artificial Sequence
  Synthesized
XXXIRF

SEQ ID NO: 11 - protein, 6 aa, Artificial Sequence
  Synthesized
XXXFRF

SEQ ID NO: 12 - protein, 6 aa, Artificial Sequence
  Synthesized
XXXWRF

SEQ ID NO: 13 - protein, 6 aa, Artificial Sequence
  Synthesized
XXXMRF

SEQ ID NO: 14 - protein, 6 aa, Artificial Sequence
  Synthesized
XXKLRF

SEQ ID NO: 15 - protein, 6 aa, Artificial Sequence
  Synthesized
XXRLRF

SEQ ID NO: 16 - protein, 6 aa, Artificial Sequence
  Synthesized
XXHLRF

SEQ ID NO: 17 - protein, 6 aa, Artificial Sequence
  Synthesized
XXTLRF

SEQ ID NO: 18 - protein, 6 aa, Artificial Sequence
  Synthesized
XXFLRF

SEQ ID NO: 19 - protein, 6 aa, Artificial Sequence
  Synthesized
XXSLRF

SEQ ID NO: 20 - protein, 6 aa, Artificial Sequence
  Synthesized
XXILRF

SEQ ID NO: 21 - protein, 6 aa, Artificial Sequence
  Synthesized
XXLLRF

SEQ ID NO: 22 - protein, 6 aa, Artificial Sequence
  Synthesized
XXALRF

SEQ ID NO: 23 - protein, 6 aa, Artificial Sequence
  Synthesized
XXWLRF

SEQ ID NO: 24 - protein, 6 aa, Artificial Sequence
  Synthesized
XXMLRF

SEQ ID NO: 25 - protein, 3 aa, Artificial Sequence
  Synthesized
FRF

SEQ ID NO: 26 - protein, 3 aa, Artificial Sequence
  Synthesized
LRF

SEQ ID NO: 27 - protein, 3 aa, Artificial Sequence
  Synthesized
WRF

SEQ ID NO: 28 - protein, 3 aa, Artificial Sequence
  Synthesized
HRF

SEQ ID NO: 29 - protein, 4 aa, Artificial Sequence
  Synthesized
FLRF

SEQ ID NO: 30 - protein, 4 aa, Artificial Sequence
  Synthesized
WLRF

SEQ ID NO: 31 - protein, 5 aa, Artificial Sequence
  Synthesized
FHLRF

SEQ ID NO: 32 - protein, 6 aa, Artificial Sequence
  Synthesized
FFKLRF

SEQ ID NO: 33 - protein, 6 aa, Artificial Sequence
  Synthesized
VFKLRF

SEQ ID NO: 34 - protein, 6 aa, Artificial Sequence
  Synthesized
HFKLRF

SEQ ID NO: 35 - protein, 6 aa, Artificial Sequence
  Synthesized
IFKLRF

SEQ ID NO: 36 - protein, 6 aa, Artificial Sequence
  Synthesized
KRKLRF

SEQ ID NO: 37 - protein, 6 aa, Artificial Sequence
  Synthesized
LFKLRF

SEQ ID NO: 38 - protein, 6 aa, Artificial Sequence
  Synthesized
YFKLRF

SEQ ID NO: 39 - protein, 7 aa, Artificial Sequence
  Synthesized
FHFKLRF

SEQ ID NO: 40 - protein, 7 aa, Artificial Sequence
  Synthesized
IHFKLRF

SEQ ID NO: 41 - protein, 6 aa, Artificial Sequence
  Synthesized
FRLKFH

SEQ ID NO: 42 - protein, 6 aa, Artificial Sequence
  Synthesized
RFKLRF

SEQ ID NO: 43 - protein, 6 aa, Artificial Sequence
  Synthesized
SFKLRF

SEQ ID NO: 44 - protein, 6 aa, Artificial Sequence
  Synthesized
MFKLRF

SEQ ID NO: 45 - protein, 6 aa, Artificial Sequence
  Synthesized
TFKLRF

SEQ ID NO: 46 - protein, 6 aa, Artificial Sequence
  Synthesized
QFKLRF

SEQ ID NO: 47 - protein, 6 aa, Artificial Sequence
  Synthesized
WFKLRF

SEQ ID NO: 48 - protein, 44 aa, Tachypleus tridentatus
  misc_feature: Tachystatin A Peptide
YSRCQLQGFNCVVRSYGLPTIPCCRGLTCRSYFPGSTYGRCQRY

SEQ ID NO: 49 - protein, 25 aa, Androctonus australis
RSVCRQIKICRRRGGCYYKCTNRPY

SEQ ID NO: 50 - protein, 13 aa, Unknown
  Synthetic Tritrpticin
VRRFPWWWPFLRR

SEQ ID NO: 51 - protein, 30 aa, Homo sapiens
  misc_feature: HNP-3 Defensin
DCYCRIPACIAGERRYGTCIYQGRLWAFCC

SEQ ID NO: 52 - protein, 38 aa, Phytolacca americana
AGCIKNGGRCNASAGPPYCCSSYCFQIAGQSYGVCKNR

SEQ ID NO: 53 - protein, 23 aa, Unknown
  Synthetic construct Magainin 2
GIGKYLHSAKKFGKAWVGEIMNS

SEQ ID NO: 54 - protein, 13 aa, Bos taurus
ILPWKWPWWPWRR

SEQ ID NO: 55 - protein, 44 aa, Heliothis virescens
DKLIGSCVWGAVNYTSDCNGECKRRGYKGGHCGSFANVNCWCET

SEQ ID NO: 56 - protein, 44 aa, Heliothis virescens; identical to SEQ ID NO: 55

SEQ ID NO: 57 - protein, 46 aa, Pisum sativum
  misc_feature: Seed of pea defensin 1 (psd1)
KTCEHLADTYRGVCFTNASCDDHCKNKAHLISGTCHNWKCFCTQNC

SEQ ID NO: 58 - protein, 18 aa, Unknown
  Synthetic Gomesin
QCRRLCYKQRCVTYCRGR

SEQ ID NO: 59 - protein, 25 aa, Bos taurus
  misc_feature: Lactoferricin B
FKCRRWQWRMKKLGAPSITCVRRAF

SEQ ID NO: 60 - protein, 12 aa, Unknown
  Synthetic PW2
HPLKQYWWRPSI

SEQ ID NO: 61 - protein, 20 aa, Homo sapiens
  misc_feature: Hepcidin 20
ICIFCCGCCHRSKCGMCCKT

SEQ ID NO: 62 - protein, 25 aa, Homo sapiens
  misc_feature: Hepcidin 25
DTHFPICIFCCGCCHRSKCGMCCKT

SEQ ID NO: 63 - protein, 30 aa, Amaranthus caudatus
VGECVRGRCPSGMCCSQFGYCGKGPKYCGR

SEQ ID NO: 64 - protein, 78 aa, Amaranthus caudatus
GYFCESCRKIIQKLEDMVGPQPNEDTVTQAASQVCDKLKILRGLCKKIMRSFLRRISWDILTGKKPQAICVDIKICKE

SEQ ID NO: 65 - protein, 23 aa, Xenopus laevis
  misc_feature: Magainin 2
GIGKFLHSAKKFGKAFVGEIMNS

SEQ ID NO: 66 - protein, 26 aa, Apis mellifera
  misc_feature: venom Melittin B
GIGAVLKVLTTGLPALISWIKRKRQQ

SEQ ID NO: 67 - protein, 21 aa, Podisus maculiventris
GSKKPVPIIYCNRRTGKCQRM

SEQ ID NO: 68 - protein, 38 aa, Mesembryanthemum crystallinum
  misc_feature: Antimicrobial peptide 1
AKCIKNGKGCREDQGPPFCCSGFCYRQVGWARGYCKNR

SEQ ID NO: 69 - protein, 13 aa, Bos taurus
  misc_feature: Melanotropin alpha (Alpha-MSH)
SYSMEHFRWGKPV

SEQ ID NO: 70 - protein, 33 aa, Oryctolagus cuniculus
  misc_feature: Corticostatin III (MCP-1)
VVCACRRALCLPRERRAGFCRIRGRIHPLCCRR

SEQ ID NO: 71 - protein, 33 aa, Oryctolagus cuniculus
  misc_feature: Corticostatin IV (MCP-2)
VVCACRRALCLPLERRAGFCRIRGRIHPLCCRR

SEQ ID NO: 72 - protein, 35 aa, Antheraea pernyi
  misc_feature: Cecropin B
KWKIFKKIEKVGRNIRNGIIKAGPAVAVLGEAKAL

SEQ ID NO: 73 - protein, 48 aa, Bos taurus
  misc_feature: Seminalplasmin
SDEKASPDKHHRFSLSRYAKLANRLANPKLLETFLSKWIGDRGNRSVK

SEQ ID NO: 74 - protein, 34 aa, Oryctolagus cuniculus
  misc_feature: NP-3A defensin
GICACRRRFCPNSERFSGYCRVNGARYVRCCSRR

SEQ ID NO: 75 - protein, 30 aa, Homo sapiens
  misc_feature: HNP-1 Defensin
ACYCRIPACIAGERRYGTCIYQGRLWAFCC

SEQ ID NO: 76 - protein, 29 aa, Homo sapiens
  misc_feature: HNP-2 Defensin
CYCRIPACIAGERRYGTCIYQGRLWAFCC

SEQ ID NO: 77 - protein, 33 aa, Homo sapiens
  misc_feature: HNP-4 Defensin
VCSCRLVFCRRTELRVGNCLIGGVSFTYCCTRV

SEQ ID NO: 78 - protein, 24 aa, Homo sapiens
  misc_feature: Histatin 5
DSHAKRHHGYKRKFHEKHHSHRGY

SEQ ID NO: 79 - protein, 32 aa, Homo sapiens
  misc_feature: Histatin 3
DSHAKRHHGYKRKFHEKHHSHRGYRSNYLYDN

SEQ ID NO: 80 - protein, 12 aa, Unknown
  Histatin 8
KFHEKHHSHRGY

SEQ ID NO: 81 - protein, 38 aa, Bos taurus
  misc_feature: Tracheal antimicrobial peptide
NPVSCVRNKGICVPIRCPGSMKQIGTCVGRAVKCCRKK

SEQ ID NO: 82 - protein, 37 aa, Mirabilis jalapa
  misc_feature: antimicrobial peptidic agent1 (MJ-antimicrobial peptidic agent1)
QCIGNGGRCNENVGPPYCCSGFCLRQPGQGYGYCKNR

SEQ ID NO: 83 - protein, 36 aa, Mirabilis jalapa
  misc_feature: antimicrobial peptidic agent2 (MJ-antimicrobial peptidic agent2)
CIGNGGRCNENVGPPYCCSGFCLRQPNQGYGVCRNR

SEQ ID NO: 84 - protein, 33 aa, Zea mays
  misc_feature: MBP-1
RSGRGECRRQCLRRHEGQPWETQECMRRCRRRG

SEQ ID NO: 85 - protein, 23 aa, Brassica napus
  misc_feature: AFP2
QKLCERPSGTWSGVCGNNNACKN

SEQ ID NO: 86 - protein, 27 aa, Brassica rapa
  misc_feature: AFP1
QKLCERPSGTWSGVCGNNNACKNQCIN

SEQ ID NO: 87 - protein, 27 aa, Brassica rapa
  misc_feature: AFP2
QKLCERPSGTWSGVCGNNNACKNQCIR

SEQ ID NO: 88 - protein, 33 aa, Phyllomedusa bicolor
  misc_feature: Adenoregulin
GLWSKIKEVGKEAAKAAAKAAGKAALGAVSEAV

SEQ ID NO: 89 - protein, 16 aa, Sus scrofa
  misc_feature: Protegrin 2
RGGRLCYCRRRFCICV

SEQ ID NO: 90 - protein, 18 aa, Sus scrofa
  misc_feature: Protegrin 3
RGGGLCYCRRRFCVCVGR

SEQ ID NO: 91 - protein, 38 aa, Macaca fascicularis
  misc_feature: Histatin 1
DSHEERHHGRHGHHKYGRKFHEKHHSHRGYRSNYLYDN

SEQ ID NO: 92 - protein, 24 aa, Xenopus laevis
  misc_feature: Peptide PGQ
GVLSNVIGYLKKLGTGALNAVLKQ

SEQ ID NO: 93 - protein, 20 aa, Rana catesbeiana
  misc_feature: Ranalexin
FLGGLIKIVPAMICAVTKKC

SEQ ID NO: 94 - protein, 30 aa, Cavia cutleri
  misc_feature: GNCP-2
RCICTTRTCRFPYRRLGTCLFQNRVYTFCC

SEQ ID NO: 95 - protein, 18 aa, Sus scrofa
  misc_feature: Protegrin 4
RGGRLCYCRGWICFCVGR

SEQ ID NO: 96 - protein, 18 aa, Sus scrofa
  misc_feature: Protegrin 5
RGGRLCYCRPRFCVCVGR

SEQ ID NO: 97 - protein, 27 aa, Bos taurus
  misc_feature: BMAP-27
GRFKRFRKKFKKLFKKLSPVIPLLHLG

SEQ ID NO: 98 - protein, 28 aa, Bos taurus
  misc_feature: BMAP-28
GGLRSLGRKILRAWKKYGPIIVPIIRIG

SEQ ID NO: 99 - protein, 39 aa, Bufo bufo gargarizans
  misc_feature: Buforin 1
AGRGKQGGKVRAKAKTRSSRAGLQFPVGRVHRLLRKGNY

SEQ ID NO: 100 - protein, 21 aa, Bufo bufo gargarizans
  misc_feature: Buforin II
TRSSRAGLQFPVGRVHRLLRK

SEQ ID NO: 101 - protein, 34 aa, Bos taurus
  misc_feature: BMAP-34
GLFRRLRDSIRRGQQKILEKARRIGERIKDIFRG

SEQ ID NO: 102 - protein, 19 aa, Trichoderma longibrachiatum
  misc_feature: Tricholongin
AGFAAQAAASLAPVAAQQL

SEQ ID NO: 103 - protein, 34 aa, Phyllomedusa sauvagei
  misc_feature: Dermaseptin 1
ALWKTMLKKLGTMALHAGKAALGAAADTISQGTQ

SEQ ID NO: 104 - protein, 45 aa, Hevea brasiliensis
  misc_feature: Pseudo-hevein (Minor hevein)
EQCGRQAGGKLCPNNLCCSQYGWCGSSDDYCSPSKNCQSNCKGGG

SEQ ID NO: 105 - protein, 33 aa, Rana rugosa
  misc_feature: Gaegurin-1
SLFSLIKAGAKFLGKNLLKQGACYAACKASKQC

SEQ ID NO: 106 - protein, 36 aa, Phyllomedusa bicolor
  misc_feature: Skin peptide tyrosine-tyrosine
YPPKPESPGEDASPEEMNKYLTALRHYINLVTRQRY

SEQ ID NO: 107 - protein, 50 aa, Penaeus vannamei
  misc_feature: Penaeidin-1
YRGGYTGPIPRPPPIGRPPLRLVVCACYRLSVSDARNCCIKFGSCCHLVK

SEQ ID NO: 108 - protein, 33 aa, Mesocricetus auratus
  misc_feature: Neutrophil defensin 1 (HANP-1)
VTCFCRRRGCASRERHIGYCRFGNTIYRLCCRR

SEQ ID NO: 109 - protein, 33 aa, Mesocricetus auratus
  misc_feature: Neutrophil defensin 3 (HANP-3)
VTCFCRRRGCASRERLIGYCRFGNTIYGLCCRR

SEQ ID NO: 110 - protein, 21 aa, Misgurnus anguillicaudatus
  misc_feature: Misgurin
RQRVEELSKFSKKGAAARRRK

SEQ ID NO: 111 - protein, 41 aa, Pharbitis nil
  misc_feature: PN-antimicrobial peptidic agent
QQCGRQASGRLCGNRLCCSQWGYCGSTASYCGAGCQSQCRS

SEQ ID NO: 112 - protein, 19 aa, Oncorhynchus mykiss
  misc_feature: Histone H2B-1(HLP-1)(Fragment)
PDPAKTAPKKGSKKAVTKA

SEQ ID NO: 113 - protein, 17 aa, Oncorhynchus mykiss
  misc_feature: Histone H2B-3(HLP-3)(Fragment)
PDPAKTAPKKKSKKAVT

SEQ ID NO: 114 - protein, 30 aa, Macaca mulatta
  misc_feature: neutrophil defensin 2 (RMAD-2)
ACYCRIPACLAGERRYGTCFYMGRVWAFCC

SEQ ID NO: 115 - protein, 36 aa, Pseudacanthotermes spiniger
  misc_feature: Termicin
ACNFQSCWATCQAQHSIYFRRAFCDRSQCKCVFVRG

SEQ ID NO: 116 - protein, 25 aa, Pseudacanthotermas spiniger
  misc_feature: Spingerin
HVDKKVADKVLLLKQLRIMRLLTRL

SEQ ID NO: 117 - protein, 13 aa, Litoria raniformis
  misc_feature: Aurein 1.1
GLFDIIKKIAESI

SEQ ID NO: 118 - protein, 30 aa, Pachycondyla goeldii
  misc_feature: Ponericin G1
GWKDWAKKAGGWLKKKGPGMAKAALKAAMQ

SEQ ID NO: 119 - protein, 24 aa, Rana berlandieri
  misc_feature: Brevinin-1BB
FLPAIAGMAAKFLPKIFCAISKKC

SEQ ID NO: 120 - protein, 20 aa, Rana clamitans
  misc_feature: Ranalexin-1CB
FLGGLMKAFPAIICAVTKKC

SEQ ID NO: 121 - protein, 31 aa, Rana clamitans
  misc_feature: Ranatuerin-2CA
GLFLDTLKGAAKDVAGKLLEGLKCKIAGCKP

SEQ ID NO: 122 - protein, 27 aa, Rana clamitans
  misc_feature: Ranatuerin-2CB
GLFLDTLKGLAGKLLQGLKCIKAGCKP

SEQ ID NO: 123 - protein, 40 aa, Ginkgo biloba
  misc_feature: Ginkbilobin
ANTAFVSSAHNTQKIPAGAPFNRNLRAMLADLRQNAAFAG

SEQ ID NO: 124 - protein, 20 aa, Basella alba
  misc_feature: Alpha-basrubrin (Fragment)
GADFQECMKEHSQKQHQHQG

SEQ ID NO: 125 - protein, 24 aa, Pseudis paradoxa
  misc_feature: Pseudin 1
GLNTLKKVFQGLHEAIKLINNHVQ

SEQ ID NO: 126 - protein, 45 aa, Parabuthus schlechteri
  misc_feature: Parabutoporin
FKLGSFLKKAWKSKLAKKLRAKGKEMLKDYAKGLLEGGSEEVPGQ

SEQ ID NO: 127 - protein, 44 aa, Opistophthalmus carinatus
  misc_feature: Opistoporin 1
GKVWDWIKSTAKKLWNSEPVKELKNTALNAAKNLVAEKIGATPS

SEQ ID NO: 128 - protein, 44 aa, Opistophthalmus carinatus
  misc_feature: Opistoporin 2
GKVWDWIKSTAKKLWNSEPVKELKNTALNAAKNFVAEKIGATPS

SEQ ID NO: 129 - protein, 12 aa, Oncorhynchus mykiss
  misc_feature: Histone H2A (Fragment)
AERVGAGAPVYL

SEQ ID NO: 130 - protein, 33 aa, Dolabella auricularia
  misc_feature: Dolabellanin B2
SHQDCYEALHKCMASHSKPFSCSMKFHMCLQQQ

SEQ ID NO: 131 - protein, 35 aa, Heliothis virescens
  misc_feature: Cecropin A
RWKVFKKIEKVGRNIRDGVIKAAPAIEVLGQAKAL

SEQ ID NO: 132 - protein, 35 aa, Homo sapiens
  misc_feature: HNP-5 Defensin
QARATCYCRTGRCATRESLSGVCEISGRLYRLCCR

SEQ ID NO: 133 - protein, 35 aa, Homo sapiens
  misc_feature: HNP-6 Defensin
STRAFTCHCRRSCYSTEYSYGTCTVMGINHRFCCL

SEQ ID NO: 134 - protein, 84 aa, Holotrichia diomphalia
  misc_feature: Holotricin 3
YGPGDGHGGGHGGGHGGGHGNGQGGGHGHGPGGGFGGGHGGGHGGGGRGGGGSGGGGSPGHGAGGGYPGGHGGGHHGGYQTHGY

SEQ ID NO: 135 - protein, 45 aa, Bos taurus
  misc_feature: Lingual antimicrobial peptide
GFTQGVRNSQSCRRNKGICVPIRCPGSMRQIGTCLGAQVKCCRRK

SEQ ID NO: 136 - protein, 29 aa, Rattus norvegicus
  misc_feature: RatNP-3
CSCRTSSCRFGERLSGACRLNGRIYRLCC

SEQ ID NO: 137 - protein, 31 aa, Cavia cutleri
  misc_feature: GNCP-1
RRCICTTRTCRFPYRRLGTCIFQNRVYTFCC

SEQ ID NO: 138 - protein, 47 aa, Penaeus vannamei
  misc_feature: Penaeidin-4a
HSSGYTRPLPKPSRPIFIRPIGCDVCYGIPSSTARLCCFRYGDCCHR

SEQ ID NO: 139 - protein, 6 aa, Bos taurus
  misc_feature: hexapeptide
RRWQWR

SEQ ID NO: 140 - protein, 18 aa, Penaeus vannamei
KWKLFKKIPKFLHLAKKF

SEQ ID NO: 141 - protein, 20 aa, Homo sapiens
  misc_feature: MUC7 20-Mer
LAHQKPFIRKSYKCLHKRCR

SEQ ID NO: 142 - protein, 21 aa, Rana nigromaculata
  misc_feature: Nigrocin 2
GLLSKVLGVGKKVLCGVSGLC

SEQ ID NO: 143 - protein, 33 aa, Rana nigromaculata
  misc_feature: Nigrocin 1
GLLDSIKGMAISAGKGALQNLLKVASCKLDKTC

SEQ ID NO: 144 - protein, 10 aa, Unknown
  lactoferrin (Lf) peptide 2
FKCRRWQWRM

SEQ ID NO: 145 - protein, 18 aa, Impatiens balsamina
  misc_feature: Ib-antimicrobial peptidic agent3
RHRCCAWGPGRKYCKRWC

SEQ ID NO: 146 - protein, 18 aa, Impatiens balsamina
  misc_feature: Ib-antimicrobial peptidic agent4
GRRCCGWGPGRRYCRRWC

SEQ ID NO: 147 - protein, 14 aa, Unknown
  Synthesis dhvar4
KRLFKKLLFSLRKY

SEQ ID NO: 148 - protein, 14 aa, Unknown
  Synthesis dhvar5
LLLFLLKKRKKRKY

SEQ ID NO: 149 - protein, 6 aa, Artificial Sequence
  Synthesized
XXXXXC

SEQ ID NO: 150 - protein, 6 aa, Artificial Sequence
  Synthesized
XXXXFC

SEQ ID NO: 151 - protein, 6 aa, Artificial Sequence
  Synthesized
XXXXNC

SEQ ID NO: 152 - protein, 6 aa, Artificial Sequence
  Synthesized
XXXXWC

SEQ ID NO: 153 - protein, 6 aa, Artificial Sequence
  Synthesized
XXXXIC

SEQ ID NO: 154 - protein, 6 aa, Artificial Sequence
  Synthesized
XXXXTC

SEQ ID NO: 155 - protein, 6 aa, Artificial Sequence
  Synthesized
XXXXYC

SEQ ID NO: 156 - protein, 6 aa, Artificial Sequence
  Synthesized
XXXXVC

SEQ ID NO: 157 - protein, 6 aa, Artificial Sequence
  Synthesized
XXXXMC

SEQ ID NO: 158 - protein, 6 aa, Artificial Sequence
  Synthesized
XXXXGC

SEQ ID NO: 159 - protein, 6 aa, Artificial Sequence
  Synthesized
XXXXEC

SEQ ID NO: 160 - protein, 6 aa, Artificial Sequence
  Synthesized
XXXXSC

SEQ ID NO: 161 - protein, 6 aa, Artificial Sequence
  Synthesized
XXXXLC

SEQ ID NO: 162 - protein, 6 aa, Artificial Sequence
  Synthesized
XXXXPC

SEQ ID NO: 163 - protein, 6 aa, Artificial Sequence
  Synthesized
XXXXHC

SEQ ID NO: 164 - protein, 6 aa, Artificial Sequence
  Synthesized
XXXXCC

SEQ ID NO: 165 - protein, 6 aa, Artificial Sequence
  Synthesized
XXXXAC

SEQ ID NO: 166 - protein, 6 aa, Artificial Sequence
  Synthesized
XXXXKC

SEQ ID NO: 167 - protein, 6 aa, Artificial Sequence
  Synthesized
XXXXQC

SEQ ID NO: 168 - protein, 6 aa, Artificial Sequence
  Synthesized
XXXXRC

SEQ ID NO: 169 - protein, 6 aa, Artificial Sequence
  Synthesized
XXXXDC

SEQ ID NO: 170 - protein, 6 aa, Artificial Sequence
  Synthesized
XXXXCY

SEQ ID NO: 171 - protein, 6 aa, Artificial Sequence
  Synthesized
XXXXCQ

SEQ ID NO: 172 - protein, 6 aa, Artificial Sequence
  Synthesized
XXXXYS

SEQ ID NO: 173 - protein, 6 aa, Artificial Sequence
  Synthesized
XXXXYD

SEQ ID NO: 174 - protein, 6 aa, Artificial Sequence
  Synthesized
XXXXTQ

SEQ ID NO: 175 - protein, 6 aa, Artificial Sequence
  Synthesized
WTFRYC

SEQ ID NO: 176 - protein, 6 aa, Artificial Sequence
  Synthesized
CYRFTW

SEQ ID NO: 177 - protein, 11 aa, Glomerella cingulata
GYFSYPHGNLF

SEQ ID NO: 178 - protein, 13 aa, Saccharomyces cerevisiae
WHWLQLKPGQPMY

SEQ ID NO: 179 - protein, 13 aa, Saccharomyces kluyveri
WHWLSFSKGQPMY

SEQ ID NO: 180 - protein, 19 aa, Artificial Sequence
  Synthesized
YNLEDHPQGDHPKLQLWHW

SEQ ID NO: 181 - protein, 15 aa, Artificial Sequence
  Synthesized
YNLEPQGPKLQLWHW

SEQ ID NO: 182 - protein, 13 aa, Artificial Sequence
  Synthesized
YMPQGPKLQLFHW

SEQ ID NO: 183 - protein, 12 aa, Artificial Sequence
  Synthesized
YMPQGPKLQLWH

SEQ ID NO: 184 - protein, 13 aa, Artificial Sequence
  Synthesized
YMPQGPRLNLWHW

SEQ ID NO: 185 - protein, 26 aa, Magnaporthe grisea
MSPSTKNIPAPVAGARAGPIHYCVIM

SEQ ID NO: 186 - protein, 24 aa, Neurospora crassa
MPSTAASTRVPQTTMNFNGYCVVM

SEQ ID NO: 187 - protein, 23 aa, Cryphonectria parasitica
MPSNTQTSNSSMGVNGYSYCVVM

SEQ ID NO: 188 - protein, 11 aa, Magnaporthe grisea
QWCPRRGQPCW

SEQ ID NO: 189 - protein, 11 aa, Neurospora crassa
QWCRIHGQSCW

SEQ ID NO: 190 - protein, 13 aa, Saccharomyces cerevisiae
WHWLQLKPGQPMY

SEQ ID NO: 191 - protein, 10 aa, Cryphonectria parasitica
WCLFHGEGCW

SEQ ID NO: 192 - protein, 4 aa, Artificial Sequence
  Synthesized
XAAC

SEQ ID NO: 193 - protein, 10 aa, Artificial Sequence
  Synthesized
WCXXGXXXCW

SEQ ID NO: 194 - protein, 6 aa, Artificial Sequence
  Synthesized
XXXXCI

SEQ ID NO: 195 - protein, 10 aa, Fusarium graminearum
WCQQKGQPCW

SEQ ID NO: 196 - protein, 10 aa, Fusarium graminearum
WCTWKGQPCW

SEQ ID NO: 197 - protein, 7 aa, Artificial Sequence
  Synthesized
FRLKFHF

SEQ ID NO: 198 - protein, 6 aa, Artificial Sequence
  Synthesized
FRLKHI

SEQ ID NO: 199 - protein, 5 aa, Artificial Sequence
  Synthesized
FRLHF